(19) United States
(12) Patent Application Publication
(10) Pub. No.: US 2013/0072468 A1
(43) Pub. Date: Mar. 21, 2013

(54) SUBSTITUTED PIPERAZINES AS CB1 ANTAGONISTS

(71) Applicants: Eric J. Gilbert, Rahway, NJ (US); William J. Greenlee, Teaneck, NJ (US); Michael W. Miller, Rahway, NJ (US); Jack D. Scott, Rahway, NJ (US); Andrew W. Stamford, Rahway, NJ (US)

(72) Inventors: Eric J. Gilbert, Rahway, NJ (US); William J. Greenlee, Teaneck, NJ (US); Michael W. Miller, Rahway, NJ (US); Jack D. Scott, Rahway, NJ (US); Andrew W. Stamford, Rahway, NJ (US)

(21) Appl. No.: 13/616,825

(22) Filed: Sep. 14, 2012

Related U.S. Application Data
(60) Provisional application No. 61/535,059, filed on Sep. 15, 2011.

(51) Int. Cl. A61K 31/496; A61K 31/495; C07D 401/04; C07D 401/06; C07D 401/14; C07D 403/06; C07D 413/06; A61K 31/5377; C07D 417/06; A61K 31/506; C07D 487/04; A61K 31/397; A61P 25/18; A61P 25/22; A61P 25/24; A61P 25/30; A61P 25/32; A61P 25/34; A61P 25/00; A61P 25/06; A61P 25/08; A61P 25/14; A61P 25/16; A61P 25/28; A61P 3/04; A61P 3/10; A61P 1/00; A61P 1/08; A61P 1/12; A61P 13/00; A61P 15/08; A61P 29/00; A61P 9/10; A61P 31/00; A61P 35/00; A61P 3/00; A61P 1/16; A61P 9/00; C07D 241/04

(52) U.S. Cl. 514/210.02; 544/395; 514/255.03; 544/389; 514/255.01; 544/360; 514/253.01; 514/253.12; 544/364; 544/357; 514/252.11; 544/121; 514/235.8; 544/295; 514/252.14

(57) ABSTRACT

Compounds of Formula (I):

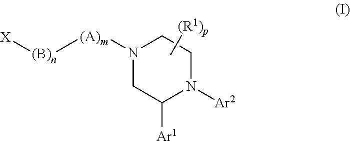

or pharmaceutically acceptable salts, solvates, or esters thereof, are useful in treating diseases or conditions mediated by CB₁ receptors, such as metabolic syndrome and obesity, neuroinflammatory disorders, cognitive disorders and psychosis, addiction (e.g., smoking cessation), gastrointestinal disorders, and cardiovascular conditions.

### CROSS-REFERENCE TO RELATED APPLICATIONS

This application is a non-provisional application that claims priority under 35 U.S.C. §119(e) of provisional application U.S. Ser. No. 61/535,059, filed Sep. 15, 2011, the contents of which are hereby incorporated by reference in its entirety.

### BACKGROUND OF THE INVENTION

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant (e.g., U.S. Pat. No. 6,432,984), can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344), neuroinflammatory disorders (e.g., Adam, et al., Expert Opin. Ther. Patents, 2002, vol. 12, no. 10, 1475-1489; U.S. Pat. No. 6,642,258), cognitive disorders and psychosis (e.g., Adam et al., Expert Opin. Ther. Pat., 2002, vol. 12, pp. 1475-1489), addiction (e.g., smoking cessation; U.S. Patent Publ. 2003/0087933), gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60; Sanofi-Aventis Publication, Bear Stearns Conference, New York, Sep. 14, 2004, pages 19-24).

However, there is still a need for improved cannabinoid agents, particularly selective CB₁ receptor antagonists, with fewer side-effects and improved efficacy. It is therefore an object of the present invention to provide substituted piperazines useful in the treatment of diseases or conditions mediated by CB₁ receptors.

WO 95/25443, U.S. Pat. No. 5,464,788, and U.S. Pat. No. 5,756,504 describe N-arylpiperazine compounds useful for treating preterm labor, stopping labor, and dysmenorrhea. However, none of the N-aryl piperazines exemplified therein have an aryl and/or heteroaryl substituent at both the 1- and 2-positions of the piperazine ring.

WO 01/02372 and U.S. Published Application No. 2003/0186960 describe cyclized amino acid derivatives for treating or preventing neuronal damage associated with neurological diseases. However, none of the 3-aryl piperazine 2-ones exemplified therein have an aryl and/or heteroaryl substituent at both the 1- and 2-positions of the piperazine ring.

WO 96/01656 describes radiolabelled substituted piperazines useful in pharmacological screening procedures, including labeled N-aryl piperazines. However, none of the N-aryl piperazines exemplified therein have an aryl and/or heteroaryl substituent at both the 1- and 2-positions of the piperazine ring.

U.S. Pat. No. 5,780,480 describes N-aryl piperazines useful as fibrinogen receptor antagonists for inhibiting the binding of fibrinogen to blood platelets, and for inhibiting the aggregation of blood platelets. However, none of the N-aryl piperazines exemplified therein have an aryl and/or heteroaryl substituent at both the 1- and 2-positions of the piperazine ring.

WO 03/008559 describes choline analogs useful for treating conditions or disorders. However, the only substituted piperazine derivative exemplified is N-(2-hydroxyethyl)-N′-(2-pyridylmethyl)-piperazine.

JP 3-200758, JP 4-26683, and JP 4-364175 describe N,N′-diarylpiperazines (i.e., 1,4-diarylpiperazines) prepared by reacting bis(2-hydroxyethyl)arylamines with an amine such as aniline. However, no 1,2-disubstituted piperazines are exemplified.

WO 97/22597 describes various 1,2,4-trisubstituted piperazine derivatives as tachykinin antagonists for treating tachykinin-mediated diseases such as asthma, bronchitis, rhinitis, cough, expectoration, etc. However, none of the 1,2,4-trisubstituted piperazine derivatives exemplified therein have an aryl and/or heteroaryl substituent at both the 1- and 2-positions of the piperazine ring.

EP 0268222, WO 88/01131, U.S. Pat. No. 4,917,896, and U.S. Pat. No. 5,073,544 describe compositions for enhancing the penetration of active agents through the skin, comprising azacyclohexanes, including N-acyl and N,N′-diacylpiperazines. However, none of the N-acyl or N,N′-diacylpiperazines exemplified therein have an aryl and/or heteroaryl substituent at both the 1- and 2-positions of the piperazine ring.

U.S. Pat. No. 6,528,529 describes compounds, including N,N′-disubstituted piperazines, which are selective for muscarinic acetylcholine receptors and are useful for treating diseases such as Alzheimer's disease. However, none of the N,N′-disubstituted piperazines exemplified therein have an aryl and/or heteroaryl substituent at both the 1- and 2-positions of the piperazine ring.

NL 6603256 describes various biologically active piperazine derivatives. However, none of the piperazine derivatives exemplified therein have a substituted aryl and/or heteroaryl substituent at both the 1- and 2-positions of the piperazine ring.

Wikström et al., J. Med. Chem. 2002, 45, 3280-3285, describe the synthesis of 1,2,3,4,10,14b-hexahydro-6-methoxy-2-methyldibnzo[c,f]pyrazine[1,2-a]azepin. However, none of the piperazine intermediates described therein have a substituted aryl and/or heteroaryl substituent at both the 1- and 2-positions of the piperazine ring.

### BRIEF SUMMARY OF THE INVENTION

In its many embodiments, the present invention provides a novel class of substituted piperazine compounds as selective CB₁ receptor antagonists for treating various conditions including, but not limited to metabolic syndrome (e.g., obesity, waist circumference, lipid profile, and insulin sensitivity), neuroinflammatory disorders, cognitive disorders, psychosis, addictive behavior, gastrointestinal disorders, and cardiovascular conditions.

The selective CB₁ receptor antagonists of the present invention are piperazine derivatives having the structure of Formula (I):

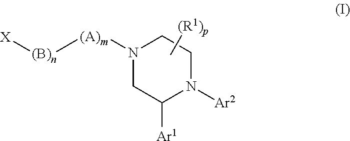

or a pharmaceutically acceptable salt, solvate, or ester thereof, wherein:
Ar¹ and Ar² are independently aryl or heteroaryl,
   wherein said aryl and heteroaryl are substituted with one or more groups Y¹,
n and m are independently 0 or 1;
A is selected from the group consisting of —C(O)—, —S(O)₂—, —C(═N—OR²)—, and —(C(R²)₂)_{q}— wherein q is 1, 2, or 3;
B is selected from the group consisting of —N(R²)—, —C(O)—, and —(C(R³)₂)ᵣ— wherein r is 1 or 2,
   with the proviso that when B is —C(O)—, then A is —C(O)— or —(C(R²)₂)_{q}—;
X is selected from the group consisting of H, alkyl, —S-alkyl, —S(O)₂-alkyl, —S(O)₂-cycloalkyl, —S(O)₂-aryl, —S(O)₂-heteroaryl, cycloalkyl, benzo-fused cycloalkyl, benzo-fused heterocycloalkyl, benzo-fused heterocycloalkenyl, heterocycloalkyl, —C(R²)═C(R²)-aryl, —C(R²)═C(R²)-heteroaryl, —OR², —O-alkylene-O-alkyl, —S-aryl, —N(R⁴)₂, —(C(R²)₂)ₛ-heteroaryl, —C(O)—O-alkyl, —C(O)-aryl, —C(O)-heteroaryl, —N═O, —C(S-alkyl)═N—S(O)₂-aryl, —C(N(R²)₂)═N—S(O)₂-aryl, and —(C(R²)₂)ₛ-aryl wherein s is 0, 1, or 2,
   wherein the heteroaryl portion of said —(C(R²)₂)ₛ-heteroaryl, the aryl portion of said —C(R²)═C(R²)-aryl, the heteroaryl portion of said —C(R²)═C(R²)— heteroaryl, the aryl portion of said —S-aryl, the aryl portion of said —S(O)₂-aryl, the heteroaryl portion of said —S(O)₂-heteroaryl, the aryl portion of said —C(O)-aryl, the heteroaryl portion of said —C(O)-heteroaryl, the aryl portion of said —(C(R²)₂)ₛ-aryl, the aryl portion of said —C(S-alkyl)═N—S(O)₂-aryl, the aryl portion of said —C(N(R²)₂)═N—S(O)₂-aryl, the benzo portion of said benzo-fused cycloalkyl, the benzo portion of said benzo-fused heterocycloalkyl, and the benzo portion of said benzo-fused heterocycloalkenyl of X are unsubstituted or substituted with one or more groups Y¹, and
   said cycloalkyl, the cycloalkyl portion of said —S(O)₂-cycloalkyl, said heterocycloalkyl, the cycloalkyl portion of said benzo-fused cycloalkyl, the heterocycloalkyl portion of said benzo-fused heterocycloalkyl, and the heterocycloalkenyl portion of said benzo-fused heterocycloalkenyl of X are unsubstituted or substituted with one or more groups Y²;
each R¹ is independently selected from the group consisting of alkyl, haloalkyl, -alkylene-N(R⁵)₂, -alkylene-OR², alkylene-N₃, -alkylene-CN, and alkylene—O—S(O)₂-alkyl; or
two R¹ groups attached to the same ring carbon atom form a carbonyl group;
p is 0, 1, 2, 3, or 4;
each R² is independently H, alkyl, or aryl,
   wherein said aryl of R² is unsubstituted or substituted with one or more groups Y¹;
each R³ is selected from the group consisting of H, alkyl, unsubstituted aryl, aryl substituted with one or more Y¹ groups, —OR², -alkylene-O-alkyl, and -alkylene-OH;
each R⁴ is selected from the group consisting of H, alkyl, aryl,—C(O)—O-alkyl, —C(O)-alkyl, —C(O)-aryl, and —S(O)₂aryl,
   wherein said aryl, the aryl portion of said —C(O)-aryl, and the aryl portion of said —S(O)₂aryl of R⁴ are unsubstituted or substituted with one or more Y¹ groups;
each R⁵ is selected from the group consisting of H, alkyl, aryl, —S(O)₂-alkyl, —S(O)₂-cycloalkyl, —S(O)₂-aryl, —C(O)—N(R²)₂, —C(O)-alkyl, and -alkylene-OH,
   wherein said aryl and the aryl portion of said —S(O)₂-aryl of R⁵ are unsubstituted or substituted with one or more Z groups;
each Y¹ is independently selected from the group consisting of alkyl, cycloalkyl, heterocycloalkyl, heterocycloalkenyl, halo, haloalkyl, benzyl, aryl, heteroaryl, —O-aryl, —S-aryl, —S(O)₂-alkyl, —S(O)₂-cycloalkyl, —S(O)₂-aryl, -alkylene-CN, —CN, —C(O)-alkyl, —C(O)-aryl, —C(O)-haloalkyl, —C(O)O-alkyl, —N(R²)C(O)-alkyl, —N(R²)C(O)—N(R²)₂, —OH, —O-alkyl, —O-haloalkyl, —O-alkylene-C(O)OH, —S-alkyl, —S-haloalkyl, -alkylene-OH, -alkylene-C(O)—O-alkyl, —O-alkylene-aryl, and —N(R⁵)₂,
   wherein said aryl, heteroaryl, the aryl portion of said —O-aryl, the aryl portion of said —S-aryl, the aryl portion of said —S(O)₂-aryl, the aryl portion of said benzyl, the aryl portion of said —C(O)-aryl, and the aryl portion of said —O-alkylene-aryl of Y¹ are unsubstituted or substituted with one or more groups Z; or
two groups Y¹ form a —O—CH₂—O— group;
each Y² is independently selected from the group consisting of alkyl, haloalkyl, aryl, -alkylene-aryl, —CN, —C(O)-alkyl, —S(O)₂-cycloalkyl, -alkylene-N(R²)₂, —C(O)-alkylene-N(R⁴)₂, —C(O)—O-alkyl, —C(O)-aryl, and —C(O)-haloalkyl,
   wherein said aryl and the aryl portion of said —C(O)-aryl of Y² are unsubstituted or substituted with one or more groups Z; or
two groups Y² form a —O—CH₂CH₂—O— group; or
two of said Y² substituents attached to the same ring carbon atom of a cycloalkyl, benzo-fused cycloalkyl, benzo-fused heterocycloalkyl, benzo-fused heterocycloalkenyl, or heterocycloalkyl ring, together with the ring carbon atom to which they are both attached, form a carbonyl group; and each Z is independently selected from the group consisting of alkyl, halo,
haloalkyl, —OH, —O-alkyl, and —CN.

In another embodiment, the present invention also provides for compositions comprising at least one selective CB₁ receptor antagonist compound of Formula (I), above, or a pharmaceutically acceptable salt, solvate, or ester thereof, and a pharmaceutically acceptable carrier.

In another embodiment, the present invention also provides for compositions comprising at least on selective CB₁ receptor antagonist compound of Formula (I), or a pharmaceutically acceptable salt, solvate, or ester thereof, in combination with at least one cholesterol lowering compound.

In yet another embodiment, the present invention also provides for a method of treating, reducing, or ameliorating metabolic syndrome, obesity, waist circumference, lipid profile, insulin sensitivity, neuroinflammatory disorders, cognitive disorders, psychosis, addictive behavior, gastrointestinal disorders, and cardiovascular conditions by administering an effective amount of at least one compound of Formula (I), or a pharmaceutically acceptable salt, solvate, or ester thereof, to a patient in need thereof.

In yet another embodiment, the present invention also provides for a method of treating vascular conditions, hyperlipidaemia, atherosclerosis, hypercholesterolemia, sitosterolemia, vascular inflammation, metabolic syndrome, stroke, diabetes, obesity and/or reducing the level of sterol(s) by administering an effective amount of a composition comprising a combination of at least one compound of Formula (I), or a pharmaceutically acceptable salt, solvate, or ester thereof, and at least one cholesterol lowering compound.

### DETAILED DESCRIPTION OF THE INVENTION

The selective CB₁ receptor antagonist compounds of the present invention are selective CB₁ receptor antagonists of mammalian CB₁ receptors, preferably human CB₁ receptors, and variants thereof. Mammalian CB₁ receptors also include CB₁ receptors found in rodents, primates, and other mammalian species.

In one embodiment, the selective CB₁ receptor antagonist compounds of the present invention are selective CB₁ receptor antagonists that bind to a CB₁ receptor with a binding affinity (K_{i(CB1)}, measured as described herein) of about 400 nM or less, or about 200 nM or less, or about 100 nM or less, or about 10 nM or less. These ranges are inclusive of all values and subranges therebetween.

In one embodiment, the selective CB₁ receptor antagonist compounds of the present invention are selective CB₁ receptor antagonists that have a ratio of CB₁ receptor affinity to CB₂ receptor affinity (K_{i(CB1)}:K_{i(CB2)}, measured as described herein) of about 1:2 or better, or about 1:10 or better, or about 1:25 or better, or about 1:50 or better, or about 1:75 or better, or about 1:90 or better. These ranges are inclusive of all values and subranges therebetween.

Thus, in one embodiment, a selective CB₁ receptor antagonist of the present invention has an affinity for the CB₁ receptor, measured as described herein, of at least 400 nM or less, and a ratio of CB₁ to CB₂ receptor affinity (i.e., (K_{i(CB1)}:K_{i(CB2)}) of at least 1:2 or better. In another embodiment the CB₁ receptor affinity is about 200 nM or less, and the (K_{i(CB1)}:K_{i(CB2)} is about 1:10 or better. In another embodiment the CB₁ affinity is about 100 nM or less, and the (K_{i(CB1)}:K_{i(CB2)} is about 1:25 or better. In another embodiment the CB₁ affinity is about 10 nM or less, and the (K_{i(CB1)}:K_{i(CB2)} is about 1:75 or better. In another embodiment the CB₁ affinity is about 10 nM or less, and the (K_{i(CB1)}:K_{i(CB2)} is about 1:90 or better. These ranges are inclusive of all values and subranges therebetween.

In one embodiment, the present invention provides for a selective CB₁ receptor antagonist compound of Formula (I), or a pharmaceutically acceptable salt, solvate, or ester thereof, wherein the various substituent groups (i.e., X, Ar¹, Ar², etc.) are as defined herein.

In another embodiment of the compound of the present invention, or a pharmaceutically acceptable salt, solvate, or ester thereof,
Ar¹ and Ar² are independently (C₆-C₁₀)aryl or (C₂-C₁₀)heteroaryl,
   wherein said (C₆-C₁₀)aryl and (C₂-C₁₀)heteroaryl are substituted with one or more groups Y¹;
n and m are independently 0 or 1;
A is selected from the group consisting of —C(O)—, —S(O)₂—, —C(═N—OR²)—, and —(C(R²)₂)_{q}— wherein q is 1, 2, or 3;
B is selected from the group consisting of —N(R²)—, —C(O)—, and —(C(R³)₂)ᵣ-
   wherein r is 1 or 2,
      with the proviso that when B is —C(O)—, then A is —C(O)— or —(C(R²)₂)_{q}—;
X is selected from the group consisting of H, (C₁-C₆)alkyl, —S—(C₁-C₆)alkyl, —S(O)₂—(C₁-C₆)alkyl, —S(O)₂—(C₃-C₁₀)cycloalkyl, —S(O)₂—(C₆-C₁₀)aryl, —S(O)₂—(C₂-C₁₀)heteroaryl, (C₃-C₁₀)cycloalkyl, benzo-fused (C₃-C₁₀)cycloalkyl, benzo-fused (C₂-C₁₀)heterocycloalkyl, benzo-fused (C₂-C₁₀)heterocycloalkenyl, (C₂-C₁₀)heterocycloalkyl, —C(R²)═C(R²)—(C₆-C₁₀)aryl, —C(R²)═C(R²)—(C₂-C₁₀)heteroaryl, —OR², —O—(C₁-C₆)alkylene—O—(C₁-C₆)alkyl, —S—(C₆-C₁₀)aryl, —N(R⁴)₂, —(C(R²)₂)_{sl —(C}₂-C₁₀)heteroaryl, —C(O)—O—(C₁-C₆)alkyl, —C(O)—(C₆-C₁₀)aryl, —C(O)—(C₂-C₁₀)heteroaryl, —N═O, —C(S—(C₁-C₆)alkyl)═N—S(O)₂—(C₆-C₁₀)aryl, —C(N(R²)₂)═N—S(O)₂—(C₆-C₁₀)aryl, and —(C(R²)₂)ₛ—(C₆-C₁₀)aryl wherein s is 0, 1, or 2,
   wherein the (C₂-C₁₀)heteroaryl portion of said —(C(R²)₂)ₛ—(C₂-C₁₀)heteroaryl, the (C₆-C₁₀)aryl portion of said —C(R²)═C(R²)—(C₆-C₁₀)aryl, the (C₂-C₁₀)heteroaryl portion of said —C(R²)═C(R²)—(C₂-C₁₀)heteroaryl, the (C₆-C₁₀)aryl portion of said —S—(C₆-C₁₀)aryl, the (C₆-C₁₀)aryl portion of said —S(O)₂—(C₆-C₁₀)aryl, the (C₂-C₁₀)heteroaryl portion of said —S(O)₂—(C₂-C₁₀)heteroaryl, the (C₆-C₁₀)aryl portion of said —C(O)—(C₆-C₁₀)aryl, the (C₂-C₁₀)heteroaryl portion of said —C(O)—(C₂-C₁₀)heteroaryl, the (C₆-C₁₀)aryl portion of said —(C(R³)₂)ₛ—(C₆-C₁₀)aryl, the (C₆-C₁₀)aryl portion of said —C(S—(C₁-C₆)alkyl)═N—S(O)₂—(C₆-C₁₀)aryl, the (C₆-C₁₀)aryl portion of said —C(N(R²)₂)═N—S(O)₂—(C₆-C₁₀)aryl, the benzo portion of said benzo-fused (C₃-C₁₀)cycloalkyl, the benzo portion of said benzo-fused (C₂-C₁₀)heterocycloalkyl, and the benzo portion of said benzo-fused (C₂-C₁₀)heterocycloalkenyl of X are unsubstituted or substituted with one or more groups Y¹, and
   said (C₃-C₁₀)cycloalkyl, the (C₃-C₁₀)cycloalkyl portion of said —S(O)₂—(C₃-C₁₀)cycloalkyl, said (C₂-C₁₀)heterocycloalkyl, the (C₃-C₁₀)cycloalkyl portion of said benzo-fused (C₃-C₁₀)cycloalkyl, the (C₂-C₁₀)heterocycloalkyl portion of said benzo-fused (C₂-C₁₀)heterocycloalkyl, and the (C₂-C₁₀)heterocycloalkenyl portion of said benzo-fused (C₂-C₁₀)heterocycloalkenyl of X are unsubstituted or substituted with one or more groups Y²;
each R¹ is independently selected from the group consisting of (C₁-C₆)alkyl, —(C₁-C₆)alkylene-N(R⁵)₂, —(C₁-C₆)alkylene-OR², (C₁-C₆)alkylene-N₃, and (C₁-C₆)alkylene—O—S(O)₂—(C₁-C₆)alkyl; or
two R¹ groups attached to the same ring carbon atom form a carbonyl group;
p is 0, 1, 2, 3, or 4;
each R² is independently H, (C₁-C₆)alkyl, or (C₆-C₁₀)aryl,
   wherein said (C₆-C₁₀)aryl of R² is unsubstituted or substituted with one or more groups Y¹;
each R³ is selected from the group consisting of H, (C₁-C₆)alkyl, unsubstituted (C₆-C₁₀)aryl, (C₆-C₁₀)aryl substituted with one or more Y¹ groups, —OR², —(C₁-C₆)alkylene—O—(C₁-C₆)alkyl, and —(C₁-C₆)alkylene-OH;
each R⁴ is selected from the group consisting of H, (C₁-C₆)alkyl, (C₆-C₁₀)aryl, —C(O)—O—(C₁-C₆)alkyl, —C(O)—(C₁-C₆)alkyl, —C(O)— (C₆-C₁₀)aryl, and —S(O)₂—(C₆-C₁₀)aryl,
wherein said (C₆-C₁₀)aryl, the (C₆-C₁₀)aryl portion of said —C(O)— (C₆-C₁₀)aryl, and the (C₆-C₁₀)aryl portion of said —S(O)₂—(C₆-C₁₀)aryl of R⁴ are unsubstituted or substituted with one or more Y¹ groups;
each R⁵ is selected from the group consisting of H, (C₁-C₆)alkyl, (C₆-C₁₀)aryl, —S(O)₂—(C₁-C₆)alkyl, —S(O)₂—(C₃-C₁₀)cycloalkyl, —S(O)₂-aryl, —C(O)—N(R²)₂, —C(O)—(C₁-C₆)alkyl, and —(C₁-C₆)alkylene-OH,
wherein said (C₆-C₁₀)aryl and the (C₆-C₁₀)aryl portion of said —S(O)₂—(C₆-C₁₀)aryl of R⁵ are unsubstituted or substituted with one or more Z groups;
each Y¹ is independently selected from the group consisting of (C₁-C₆)alkyl, (C₃-C₁₀)cycloalkyl, (C₂-C₁₀)heterocycloalkyl, (C₂-C₁₀)heterocycloalkenyl, halo, (C₁-C₆)haloalkyl, benzyl, (C₆-C₁₀)aryl, (C₂-C₁₀)heteroaryl, —O—(C₆-C₁₀)aryl, —S—(C₆-C₁₀)aryl, —S(O)₂—(C₁-C₆)alkyl, —S(O)₂—(C₃-C₁₀)cycloalkyl, —S(O)₂—(C₆-C₁₀)aryl, —(C₁-C₆)alkylene-CN, —CN, —C(O)—(C₁-C₆)alkyl, —C(O)—(C₆-C₁₀)aryl, —C(O)—(C₁-C₆)alkyl, —C(O)—(C₁-C₆)haloalkyl, —C(O)O—(C₁-C₆)alkyl, —N(R²)C(O)—(C₁-C₆)alkyl, —N(R²)C(O)—N(R²)₂, —OH, —O—(C₁-C₆)alkyl, —O—(C₁-C₆)haloalkyl, —O—(C₁-C₆)alkylene-C(O)OH, —S—(C₁-C₆)alkyl, —S—(C₁-C₆)haloalkyl, —(C₁-C₆)alkylene-OH, -(C₁-C₆)alkylene-C(O)—O—(C₁-C₆)alkyl, —O—(C₁-C₆)alkylene-(C₆-C₁₀)aryl, and —N(R⁵)₂,
   wherein said (C₆-C₁₀)aryl, said (C₂-C₁₀)heteroaryl, the (C₆-C₁₀)aryl portion of said —O—(C₆-C₁₀)aryl, the (C₆-C₁₀)aryl portion of said —S—(C₆-C₁₀)aryl, the (C₆-C₁₀)aryl portion of said —S(O)₂—(C₆-C₁₀)aryl, said benzyl, the (C₆-C₁₀)aryl portion of said —C(O)—(C₆-C₁₀)aryl, and the (C₆-C₁₀)aryl portion of said —O—(C₁-C₆)alkylene-(C₆-C₁₀)aryl of Y¹ are unsubstituted or substituted with one or more groups Z; or
two groups Y¹ form a —O—CH₂—O— group;
each Y² is independently selected from the group consisting of (C₁-C₆)alkyl, (C₁-C₆)haloalkyl, (C₆-C₁₀)aryl, —(C₁-C₆)alkylene-(C₆-C₁₀)aryl, —CN, —C(O)—(C₁-C₆)alkyl, —S(O)₂—(C₃-C₁₀)cycloalkyl, —(C₁-C₆)alkylene-N(R²)₂, —C(O)—(C₁-C₆)alkylene-N(R⁴)₂, —C(O)—O—(C₁-C₆)alkyl, —C(O)— (C₆-C₁₀)aryl, and —C(O)— (C₁-C₆)haloalkyl,
   wherein said aryl and the (C₆-C₁₀)aryl portion of said —C(O)— (C₆-C₁₀)aryl of Y² are unsubstituted or substituted with one or more groups Z; or
two groups Y² form a —O—CH₂CH₂—O— group; or
two of said Y² substituents attached to the same ring carbon atom of a (C₃-C₁₀)cycloalkyl, benzo-fused (C₃-C₁₀)cycloalkyl, benzo-fused (C₂-C₁₀)heterocycloalkyl, benzo-fused (C₂-C₁₀)heterocycloalkenyl, or (C₂-C₁₀)heterocycloalkyl ring, together with the ring carbon atom to which they are both attached, form a carbonyl group; and
   Z is independently selected from the group consisting of (C₁-C₆)alkyl, halo, (C₁-C₆)haloalkyl, —OH, —O—(C₁-C₆)alkyl, and —CN.

In another embodiment of the compounds of the present invention, or pharmaceutically acceptable salts, solvates, or esters thereof, Ar¹ and Ar² are independently aryl substituted with one or more groups Y¹.

In another embodiment of the compounds of the present invention, or pharmaceutically acceptable salts, solvates, or esters thereof, Ar¹ and Ar² are independently aryl substituted with one or more groups Y¹, m is 1; and A is —C(O)—.

In another embodiment of the compounds of the present invention, or pharmaceutically acceptable salts, solvates, or esters thereof, Ar¹ and Ar² are independently aryl substituted with one or more groups Y¹, m is 1; n is 0; and A is —C(O)—.

In another embodiment of the compounds of the present invention, or pharmaceutically acceptable salts, solvates, or esters thereof, Ar¹ and Ar² are independently aryl substituted with one or more groups Y¹, m is 1; n is 1; B is —NH—; and A is —C(O)—.

In another embodiment of the compounds of the present invention, or pharmaceutically acceptable salts, solvates, or esters thereof, Ar¹ and Ar² are independently aryl substituted with one or more groups Y¹, m is 1; n is 1; B is —(C(R²)₂)ᵣ— wherein r is 1 or 2; and A is —C(O)—.

In another embodiment of the compounds of the present invention, or pharmaceutically acceptable salts, solvates, or esters thereof, Ar¹ and Ar² are independently aryl substituted with one or more groups Y¹, m is 1; n is 1; B is —N(R²)— wherein r is 1 or 2; and A is —C(O)—.

In another embodiment of the compounds of the present invention, or pharmaceutically acceptable salts, solvates, or esters thereof, m is 1; and A is —S(O)₂—.

In another embodiment of the compounds of the present invention, or pharmaceutically acceptable salts, solvates, or esters thereof, m is 1; n is 0; and A is —S(O)₂—.

In another embodiment of the compounds of the present invention, or pharmaceutically acceptable salts, solvates, or esters thereof, m is 1; n is 1; B is —N(R²)—; and A is —S(O)₂—.

In another embodiment of the compounds of the present invention, or pharmaceutically acceptable salts, solvates, or esters thereof, m is 1; A is —C(═N—OR²)—; and n is 0.

In another embodiment of the compounds of the present invention, or pharmaceutically acceptable salts, solvates, or esters thereof, m is 1; A is —(C(R²)₂)_{q}— wherein q is 1, 2, or 3; and n is 0.

In another embodiment of the compounds of the present invention, or pharmaceutically acceptable salts, solvates, or esters thereof, X is aryl or heteroaryl, and said aryl or heteroaryl of X is unsubstituted or substituted with one or more Y¹ groups; m is 1; A is —(C(R²)₂)_{q}— wherein q is 1, 2, or 3; and n is 0.

In another embodiment of the compounds of the present invention, or pharmaceutically acceptable salts, solvates, or esters thereof, X is aryl or heteroaryl, and said aryl or heteroaryl of X is unsubstituted or substituted with one or more Y¹ groups; m is 1; A is —(C(R²)₂)_{q}— wherein q is 1 or 2; and n is 0.

In another embodiment of the compounds of the present invention, or pharmaceutically acceptable salts, solvates, or esters thereof, m is 0; B is —(C(R³)₂)ᵣ— wherein r is 1, 2, or 3; and A is —(C(R²)₂)_{q}— wherein q is 1, 2, or 3.

In another embodiment of the compounds of the present invention, or pharmaceutically acceptable salts, solvates, or esters thereof, m and n are both 1; B is —(C(R³)₂)ᵣ— wherein r is 1, 2, or 3; and n is 1.

In another embodiment of the compounds of the present invention, or pharmaceutically acceptable salts, solvates, or esters thereof, m and n are both 0.

In another embodiment of the compounds of the present invention, or pharmaceutically acceptable salts, solvates, or esters thereof, X is —(C(R²)₂)ₛ-aryl wherein s is 0, 1, or 2.

In another embodiment of the compounds of the present invention, or pharmaceutically acceptable salts, solvates, or esters thereof, X is heteroaryl.

In another embodiment of the compounds of the present invention, or pharmaceutically acceptable salts, solvates, or esters thereof, X is cycloalkyl.

In another embodiment of the compounds of the present invention, or pharmaceutically acceptable salts, solvates, or esters thereof, X is heterocycloalkyl.

In another embodiment of the compounds of the present invention, or pharmaceutically acceptable salts, solvates, or esters thereof, X is alkyl.

In another embodiment of the compounds of the present invention, or pharmaceutically acceptable salts, solvates, or esters thereof, X is —N(R⁴)₂.

In another embodiment of the compounds of the present invention, or pharmaceutically acceptable salts, solvates, or esters thereof, m and n are both 1; B is —(C(R³)₂)ᵣ— wherein r is 1, 2, or 3; and A is —C(O)—.

In another embodiment of the compounds of the present invention, or pharmaceutically acceptable salts, solvates, or esters thereof, m and n are both 1; A is —C(O)—; and B is —NH—.

In yet another embodiment the compounds of the present invention, or pharmaceutically acceptable salts, solvates, or esters thereof, have the following Formula (IA):

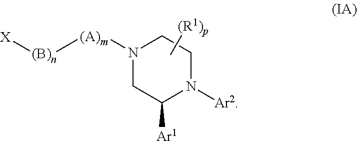

In yet another embodiment the compounds of the present invention, or pharmaceutically acceptable salts, solvates, or esters thereof, have the following Formula (IB):

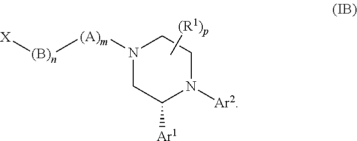

In yet another embodiment the compounds of the present invention, or pharmaceutically acceptable salts, solvates, or esters thereof, have the following Formula (IC):

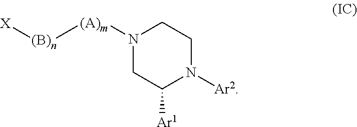

In yet another embodiment, the compounds of the present invention, or pharmaceutically acceptable salts, solvates, or esters thereof, have the Formula (IC) above, wherein m is 1 and A is —C(O)—.

In yet another embodiment, the compounds of the present invention, or pharmaceutically acceptable salts, solvates, or esters thereof, have the Formula (IC) above, wherein m is 1, n is 0, and A is —C(O)—.

In yet another embodiment, the compounds of the present invention, or pharmaceutically acceptable salts, solvates, or esters thereof, have the Formula (IC) above, wherein m is 1, n is 1, A is —C(O)—, and B is —N(R²)—.

In yet another embodiment, the compounds of the present invention, or pharmaceutically acceptable salts, solvates, or esters thereof, have the Formula (IC) above, wherein m is 1 and A is —S(O)₂—.

In yet another embodiment, the compounds of the present invention, or pharmaceutically acceptable salts, solvates, or esters thereof, have the Formula (IC) above, wherein m is 1, n is 0, and A is —S(O)₂—.

In yet another embodiment, the compounds of the present invention, or pharmaceutically acceptable salts, solvates, or esters thereof, have the Formula (IC) above, wherein m is 1, nil, B is —N(R²)—, and A is —S(O)₂—.

In yet another embodiment, the compounds of the present invention, or pharmaceutically acceptable salts, solvates, or esters thereof are selected from the group consisting of:

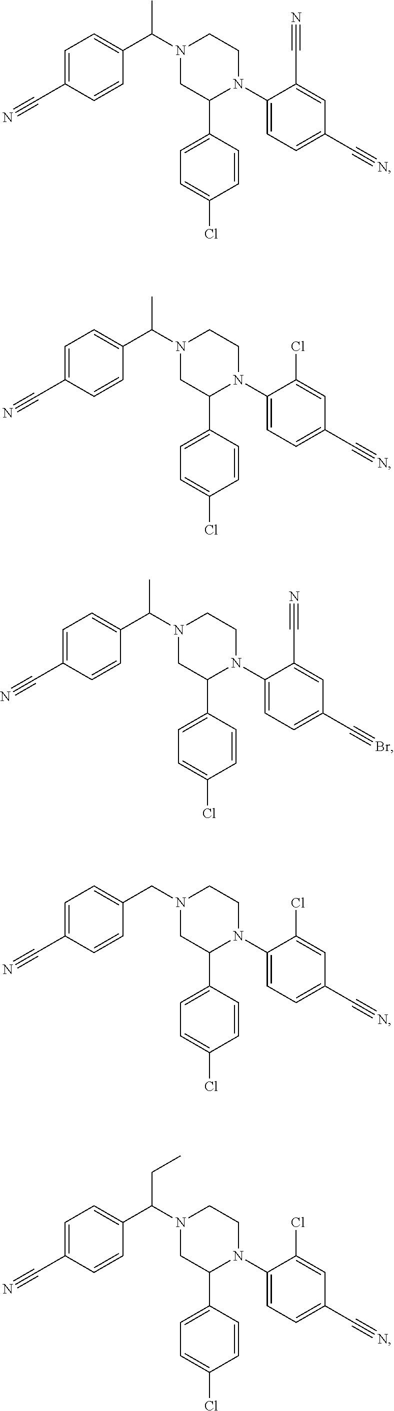

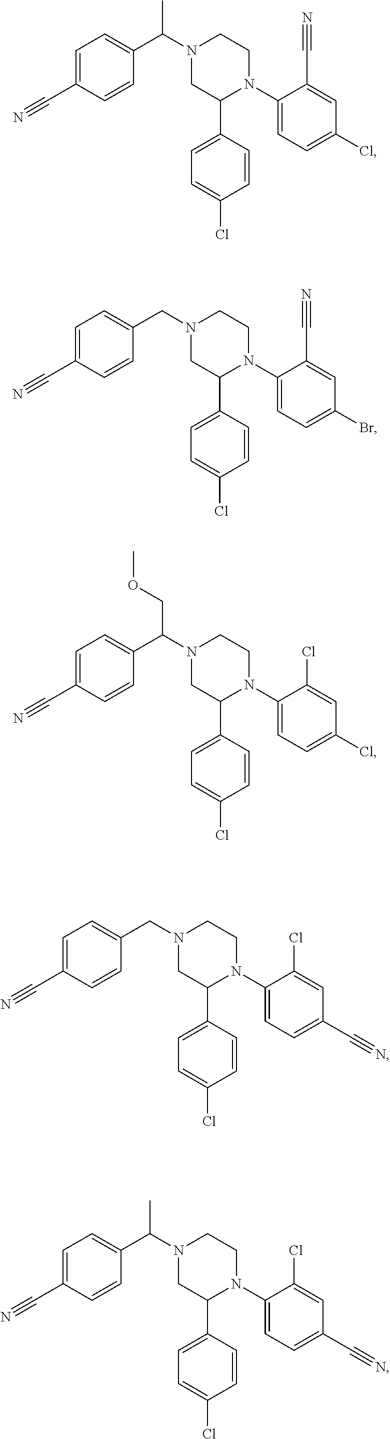 [SMILES: CC(c1ccc(C#N)cc1)N1CCN(c2ccc(Cl)cc2C#N)C(c2ccc(Cl)cc2)C1 ; N#Cc1ccc(CN2CCN(c3ccc(Br)cc3C#N)C(c3ccc(Cl)cc3)C2)cc1 ; COCC(c1ccc(C#N)cc1)N1CCN(c2ccc(Cl)cc2Cl)C(c2ccc(Cl)cc2)C1 ; N#Cc1ccc(CN2CCN(c3ccc(C#N)cc3Cl)C(c3ccc(Cl)cc3)C2)cc1 ; CC(c1ccc(C#N)cc1)N1CCN(c2ccc(C#N)cc2Cl)C(c2ccc(Cl)cc2)C1]

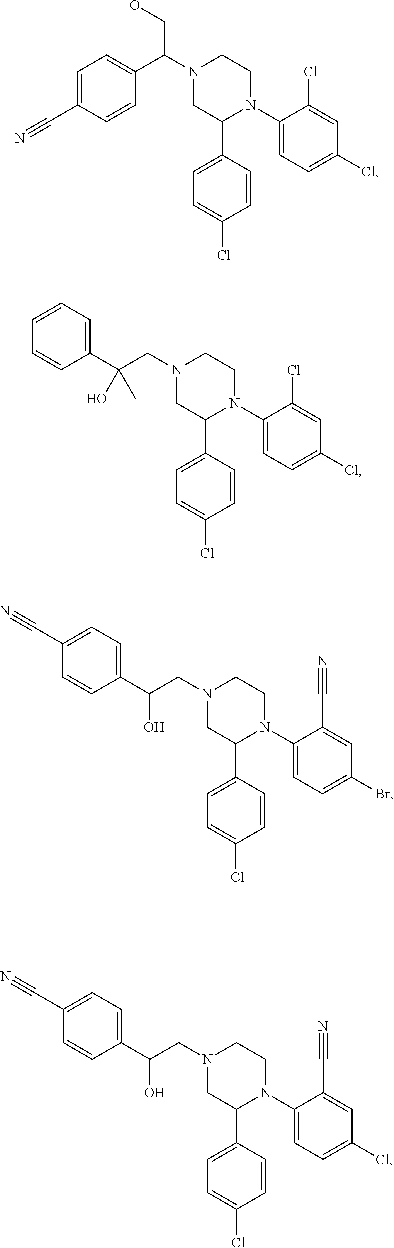 [SMILES: N#Cc1ccc(C(CO)N2CCN(c3ccc(Cl)cc3Cl)C(c3ccc(Cl)cc3)C2)cc1 ; CC(O)(CN1CCN(c2ccc(Cl)cc2Cl)C(c2ccc(Cl)cc2)C1)c1ccccc1 ; N#Cc1ccc(C(O)CN2CCN(c3ccc(Br)cc3C#N)C(c3ccc(Cl)cc3)C2)cc1 ; N#Cc1ccc(C(O)CN2CCN(c3ccc(Cl)cc3C#N)C(c3ccc(Cl)cc3)C2)cc1]

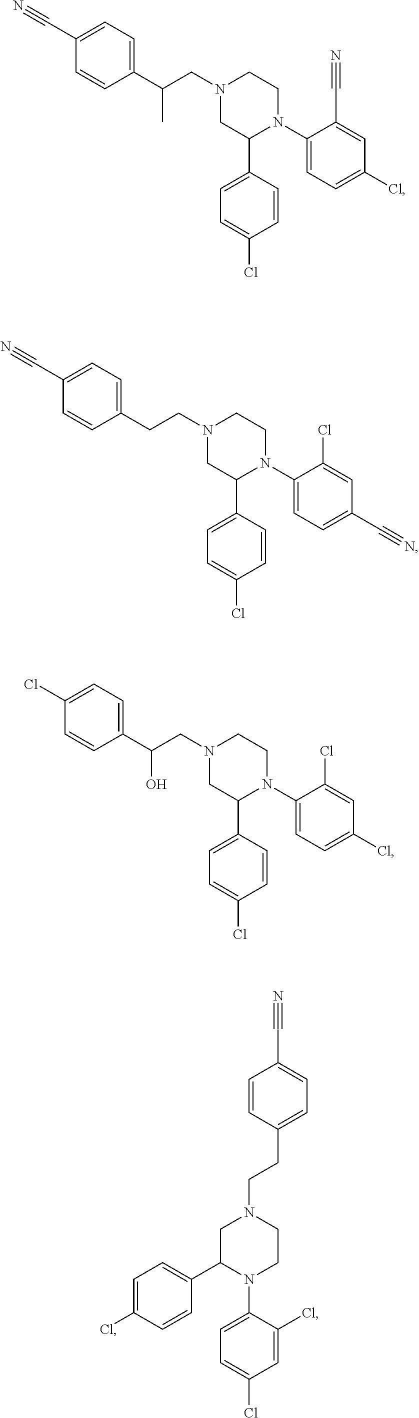 [SMILES: CC(CN1CCN(c2ccc(Cl)cc2C#N)C(c2ccc(Cl)cc2)C1)c1ccc(C#N)cc1 ; N#Cc1ccc(CCN2CCN(c3ccc(C#N)cc3Cl)C(c3ccc(Cl)cc3)C2)cc1 ; OC(CN1CCN(c2ccc(Cl)cc2Cl)C(c2ccc(Cl)cc2)C1)c1ccc(Cl)cc1 ; N#Cc1ccc(CCN2CCN(c3ccc(Cl)cc3Cl)C(c3ccc(Cl)cc3)C2)cc1]

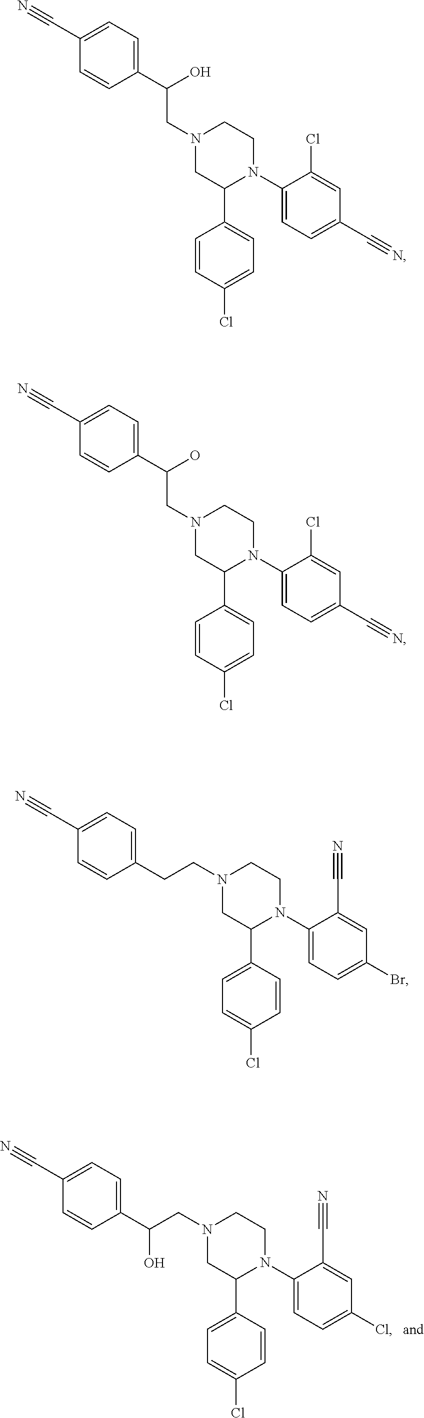 [SMILES: N#Cc1ccc(C(O)CN2CCN(c3ccc(C#N)cc3Cl)C(c3ccc(Cl)cc3)C2)cc1 ; N#Cc1ccc(C(O)CN2CCN(c3ccc(C#N)cc3Cl)C(c3ccc(Cl)cc3)C2)cc1 ; N#Cc1ccc(CCN2CCN(c3ccc(Br)cc3C#N)C(c3ccc(Cl)cc3)C2)cc1 ; N#Cc1ccc(C(O)CN2CCN(c3ccc(Cl)cc3C#N)C(c3ccc(Cl)cc3)C2)cc1 ; "and"]

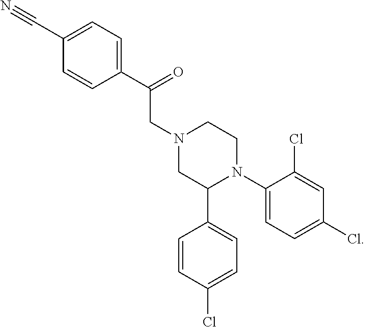 [SMILES: N#Cc1ccc(C(=O)CN2CCN(c3ccc(Cl)cc3Cl)C(c3ccc(Cl)cc3)C2)cc1]

One of ordinary skill will recognize that the compounds shown above have stereogenic centers. Thus, the compounds shown above include all possible stereoisomers.

In yet another embodiment, the compounds of the present invention, or pharmaceutically acceptable salts, solvates, or esters thereof are selected from the group consisting of:

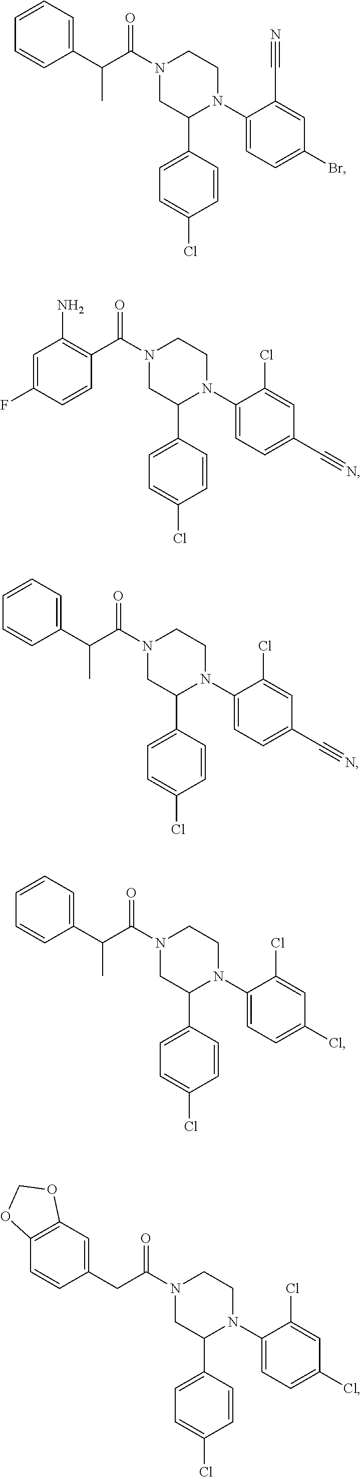 [SMILES: CC(C(=O)N1CCN(c2ccc(Br)cc2C#N)C(c2ccc(Cl)cc2)C1)c1ccccc1 ; N#Cc1ccc(N2CCN(C(=O)c3ccc(F)cc3N)CC2c2ccc(Cl)cc2)c(Cl)c1 ; CC(C(=O)N1CCN(c2ccc(C#N)cc2Cl)C(c2ccc(Cl)cc2)C1)c1ccccc1 ; CC(C(=O)N1CCN(c2ccc(Cl)cc2Cl)C(c2ccc(Cl)cc2)C1)c1ccccc1 ; O=C(Cc1ccc2c(c1)OCO2)N1CCN(c2ccc(Cl)cc2Cl)C(c2ccc(Cl)cc2)C1]

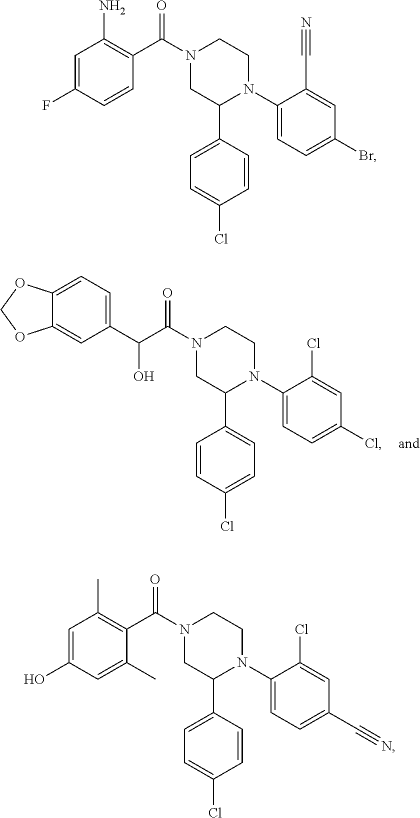 [SMILES: N#Cc1cc(Br)ccc1N1CCN(C(=O)c2ccc(F)cc2N)CC1c1ccc(Cl)cc1 ; O=C(C(O)c1ccc2c(c1)OCO2)N1CCN(c2ccc(Cl)cc2Cl)C(c2ccc(Cl)cc2)C1 ; "and" ; Cc1cc(O)cc(C)c1C(=O)N1CCN(c2ccc(C#N)cc2Cl)C(c2ccc(Cl)cc2)C1]

or a pharmaceutically acceptable salt, solvate, or ester thereof. One of ordinary skill will recognize that the compounds shown above have stereogenic centers. Thus, the compounds shown above include all possible stereoisomers.

Ar¹ and Ar² are independently aryl or heteroaryl, wherein said aryl and heteroaryl are substituted with one or more groups Y¹. Non-limiting examples of said aryl of Ar¹ and/or Ar² include, for example, phenyl, naphthyl, pyridyl (e.g., 2-, 3-, and 4-pyridyl), quinolyl, etc. substituted with one or more (e.g., 1, 2, 3, or 4) Y¹ groups as defined herein.

A is selected from the group consisting of —C(O)—, —S(O)₂—, —C(═N—OR²)—, and —(C(R²)₂)_{q}— wherein q is 1, 2, or 3. Non-limiting examples of A when A is —(C(R²)₂)_{q}— include, for example, —CH₂—, —CH₂CH₂—, —CH(CH₃)—, —C(CH₃)₂—, —CH₂CH₂CH₂—, —CH(CH₃)CH₂—, —CH₂CH(CH₃)—, —CH(CH₃)—(CH₂)₂—, —(CH₂)₂—CH(CH₃)—, —CH(phenyl)-CH₂—, —CH₂—CH(phenyl)-, —CH(phenyl)-, etc. Non-limiting examples of A when A is —C(═N—OR²)— include —C(═N—OH)—, —C(═N—OCH₃)—, —C(═N—OCH₂CH₃)—, —C(═N—OCH(CH₃)₂)—, —C(═N—OC(CH₃)₃)—, —C(═N—O-phenyl), etc.

B is selected from the group consisting of —N(R²)—, —C(O)—, and —(C(R³)₂)ᵣ— wherein r is 1 or 2. Non-limiting examples of B when B is —(C(R³)₂)ᵣ— include, for example, —CH₂—, —CH₂CH₂—, —CH(CH₃)—, —C(CH₃)₂—, —CH(CH(CH₃)₂)—, —CH(CH₂CH(CH₃)₂)—, —CH₂CH₂CH₂—, —CH(CH₃)CH₂—, —CH₂CH(CH₃)—, —CH(CH₃)—(CH₂)₂—, —(CH₂)₂—CH(CH₃)—, —CH(phenyl)-CH₂—, —CH₂—CH(phenyl)-, —CH(phenyl)-, —CH(OH)—, —C(CH₃)(OH)—, —CH(OH)CH₂—, —CH₂CH(OH)—, —CH(OH)CH₂CH(CH₃)—, —CH(CH(OH)(CH₃))—, —CH(CH₃)CH₂CH(OH)—, —CH(CH₂OH)—, —CH(OCH₃)—, —CH(OCH₃)CH₂—, —CH₂CH(OCH₃)—, —CH(OCH₃)CH₂CH(CH₃)—, —CH(CH₃)CH₂CH(OCH₃)—, —CH(CH₂OCH₃)—, —CH(OCH₃)—, —CH(OCH₂CH₃)CH₂—, —CH₂CH(OCH₂CH₃)—, —CH(OCH₂CH₃)CH₂CH(CH₃)—, —CH(CH₃)CH₂CH(OCH₂CH₃)—, —CH(CH₂OCH₂CH₃)—, etc. Non-limiting examples of B when B is —N(R²)— include —NH—, —N(alkyl)-, —N(aryl)-, wherein the terms “alkyl” and “aryl” are as defined above.

X is selected from the group consisting of H, alkyl, —S-alkyl, —S(O)₂-alkyl, —S(O)₂-cycloalkyl, —S(O)₂-aryl, —S(O)₂-heteroaryl, cycloalkyl, benzo-fused cycloalkyl, benzo-fused heterocycloalkyl, benzo-fused heterocycloalkenyl, heterocycloalkyl, —C(R²)═C(R²)-aryl, —C(R²)═C(R²)-heteroaryl, —OR², —O-alkylene-O-alkyl, —S-aryl, —N(R⁴)₂, —(C(R²)₂)ₛ-heteroaryl, —C(O)—O-alkyl, —C(O)-aryl, —C(O)-heteroaryl, —N═O, —C(S-alkyl)═N—S(O)₂-aryl, —C(N(R²)₂)═N—S(O)₂-aryl, and —(C(R²)₂)ₛ-aryl wherein s is 0, 1, or 2. Non-limiting examples of X when X is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of X when X is —S-alkyl include —S-methyl, —S-ethyl, —S-(n-propyl), —S-(iso-propyl), —S-(n-butyl), —S-(iso-butyl), —S-(sec-butyl), —S-(tert-butyl), —S-(n-pentyl), —S-(iso-pentyl), —S-(neo-pentyl), —S-(n-hexyl), —S-(iso-hexyl), etc. Non-limiting examples of X when X is —S(O)₂-alkyl include —S(O)₂-methyl, —S(O)₂-ethyl, —S(O)₂-(n-propyl), —S(O)₂-(iso-propyl), —S(O)₂-(n-butyl), —S(O)₂-(iso-butyl), —S(O)₂-(sec-butyl), —S(O)₂-(tert-butyl), —S(O)₂-(n-pentyl), —S(O)₂-(iso-pentyl), —S(O)₂-(neo-pentyl), —S(O)₂-(n-hexyl), —S(O)₂-(iso-hexyl), etc. Non-limiting examples of X when X is —S(O)₂-cycloalkyl include —S(O)₂-cyclopropyl, —S(O)₂-cyclobutyl, —S(O)₂-cyclopentyl, —S(O)₂-cyclohexyl, —S(O)₂-cycloheptyl, —S(O)₂-adamantyl, —S(O)₂-(bicyclo[2.1.1]hexanyl), —S(O)₂-(bicyclo[2.2.1]heptenyl), —S(O)₂-(bicyclo[3.1.1]heptenyl), —S(O)₂-(bicyclo[2.2.2]octenyl), —S(O)₂-(bicyclo[3.2.1]octenyl), etc. Non-limiting examples of X when X is —S(O)₂-aryl includes —S(O)₂-phenyl, —S(O)₂-naphthyl, etc. Non-limiting examples of X when X is —S(O)₂-heteroaryl include —S(O)₂-pyridyl, —S(O)₂-azaindolyl, —S(O)₂-benzimidazolyl, —S(O)₂-benzofuranyl, —S(O)₂-furanyl, —S(O)₂-indolyl, etc. Non-limiting examples of X when X is cycloalkyl include cyclopropyl, cyclobutyl, cyclopentyl, cyclohexyl, cycloheptyl, adamantyl, bicyclo[2.1.1]hexanyl, bicyclo[2.2.1]heptenyl, bicyclo[3.1.1]heptenyl, bicyclo[2.2.2]octenyl, bicyclo[3.2.1]octenyl, etc. Non-limiting examples of X when X is benzo-fused cycloalkyl include 1,2,3,4-tetrahydronaphthyl, indanyl, bicyclo[4.2.0]octa-1,3,5-trienyl, etc. Non-limiting examples of X when X is benzo-fused heterocycloalkyl includes 3,4-dihydro-2H-benzo[1,4]oxazinyl, chromanyl, 2,3-dihydro-1H-indolyl, 2,3-dihydro-1H-isoindolyl, 2,3-dihydro-benzofuranyl, 1,3-dihydro-isobenzofuranyl, 2,3-dihydro-benzo[b]thiophenyl, 1,3-dihydro-benzo[c]thiophenyl, etc. Non-limiting examples of X when X is benzo-fused heterocycloalkenyl include 2H-benzo[1,4]oxazinyl, 4H-chromenyl, 4H-chromenyl, 3H-indolyl, 1H-isoindolyl, 4H-benzo[1,4]oxazinyl, etc. Non-limiting examples of X when X is heterocycloalkyl include morpholinyl, piperazinyl, piperidinyl, pyrrolidinyl, tetrahydrofuranyl, tetrahydrothiophenyl, tetrahydropyranyl, azetidinyl, etc. When X is —C(R²)═C(R²)-aryl, non-limiting examples of X include —CH═CH-aryl, —C(CH₃)═CH-aryl, —CH═C(CH₃)-aryl, —C(CH₃)═C(CH₃)-aryl, —C(phenyl)=CH-aryl, —C(phenyl)=C(CH₃)-aryl, where “aryl” includes, for example, the aryl groups listed above. When X is —C(R²)═C(R²)-heteroaryl, non-limiting examples of X include —CH═CH-heteroaryl, —C(CH₃)═CH-heteroaryl, —CH═C(CH₃)— heteroaryl, —C(CH₃)═C(CH₃)— heteroaryl, —C(phenyl)=CH-heteroaryl, —C(phenyl)=C(CH₃)— heteroaryl, where “heteroaryl” includes, for example, the heteroaryl groups listed above. When X is —OR², R² is defined as described herein. Thus, X includes —OH, —O-alkyl (where the term “alkyl” is defined as described above), and —O-aryl (where the term “aryl” is defined as described above). When X is —O-alkylene-O-alkyl, non-limiting examples of X include —O—CH₂—O—CH₃, —O—CH(CH₃)—O—CH₃, —O—CH₂CH₂—O—CH₃, —O—CH₂CH₂—O—CH₂CH₃, —O—CH(OCH₃)CH₂CH(CH₃)₂, —O—CH(CH₃)CH₂CH₂—O—CH₃, —O—CH₂CH₂—O—CH₂CH₃, etc. Non-limiting examples of X when X is —S-aryl includes —S-phenyl, —S-naphthyl, etc. Non-limiting examples of X when X is —N(R⁴)₂ include —NH₂, —NH(alkyl), —N(alkyl)₂, —NH(aryl), —N(alkyl)(aryl), —N(aryl)₂, —NH—C(O)—O-alkyl, —N(alkyl)-C(O)—O-alkyl, —N(aryl)-C(O)—O-alkyl, —NH—C(O)alkyl, —N(alkyl)-C(O)alkyl, and —N(aryl)-C(O)alkyl where the terms “alkyl” and “aryl” are defined as described above. Non-limiting examples of X when X is —(C(R²)₂)ₛ-heteroaryl, include heteroaryl, —C(R²)₂-heteroaryl, —(C(R²)₂)₂-heteroaryl, where R² and the term “heteroaryl” are as defined herein, and “—(C(R²)₂)ₛ-” includes —CH₂—, —CH₂CH₂—, —CH(CH₃)—, —C(CH₃)₂—, —CH(CH(CH₃)₂)—, —CH(CH₂CH(CH₃)₂)—, —CH₂CH₂CH₂—, —CH(CH₃)CH₂—, —CH₂CH(CH₃)—, —CH(CH₃)—(CH₂)₂—, —(CH₂)₂—CH(CH₃)—, —CH(phenyl)-CH₂—, —CH₂—CH(phenyl)-, —CH(phenyl)-, etc. Non-limiting examples of X when X is —C(O)—O-alkyl include —C(O)—O-(methyl), —C(O)—O-(ethyl), —C(O)—O-(n-propyl), —C(O)—O-(iso-propyl), —C(O)—O-(n-butyl), —C(O)—O-(iso-butyl), —C(O)—O-(sec-butyl), —C(O)—O-(tert-butyl), —C(O)—O-(n-pentyl), —C(O)—O-(iso-pentyl), —C(O)—O-(neo-pentyl), etc. Non-limiting examples of X when X is —C(O)-aryl include —C(O)-phenyl, —C(O)-naphthyl, etc. Non-limiting examples of X when X is —C(O)-heteroaryl include —C(O)-pyridyl, —C(O)-azaindolyl, —C(O)-benzimidazolyl, —C(O)-benzothiophenyl, —C(O)-furanyl, —C(O)-furazanyl, —C(O)-indolyl, —C(O)-isoquinolyl, etc. When X is —C(S-alkyl)═N—S(O)₂-aryl, the “alkyl” and “aryl” portions thereof can independently include any of the alkyl and aryl groups described herein. Likewise, when X is —C(N(R²)₂)═N—S(O)₂-aryl said R² groups and the “aryl” portion are as defined herein. Non-limiting examples of X when X is —(C(R²)₂)ₛ-aryl, include aryl, —C(R²)₂-aryl, —(C(R²)₂)₂-aryl, where R² and the term “aryl” are as defined herein, and “—(C(R²)₂)ₛ-” is as defined above. Said heteroaryl, the heteroaryl portion of said —(C(R²)₂)ₛ-heteroaryl, the aryl portion of said —C(R²)═C(R²)-aryl, the heteroaryl portion of said —C(R²)═C(R²)-heteroaryl, the aryl portion of said —S-aryl, the aryl portion of said —S(O)₂-aryl, the heteroaryl portion of said —S(O)₂-heteroaryl, the aryl portion of said —C(O)-aryl, the heteroaryl portion of said —C(O)-heteroaryl, the aryl portion of said —(C(R²)₂)ₛ-aryl, the benzo portion of said benzo-fused cycloalkyl, the benzo portion of said benzo-fused heterocycloalkyl, and the benzo portion of said benzo-fused heterocycloalkenyl of X are unsubstituted or substituted with one or more groups Y¹, where Y¹ is defined as described herein, and said cycloalkyl, the cycloalkyl portion of said —S(O)₂-cycloalkyl, said heterocycloalkyl, the cycloalkyl portion of said benzo-fused cycloalkyl, the heterocycloalkyl portion of said benzo-fused heterocycloalkyl, and the heterocycloalkenyl portion of said benzo-fused heterocycloalkenyl of X are unsubstituted or substituted with one or more groups Y² where Y² is defined as described herein.

Each R¹ is independently selected from the group consisting of alkyl, haloalkyl, -alkylene-N(R⁵)₂, -alkylene-OR², alkylene-N₃, and alkylene—O—S(O)₂-alkyl. Non-limiting examples of R¹ when R¹ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R¹ when R¹ is haloalkyl include —CF₃, —CHF₂, —CH₂F, —CH₂CF₃, —CF₂CF₃, —CH₂Br, —CH₂Cl, —CCl₃, etc. When R¹ is alkylene-N₃ or alkylene—O—S(O)₂-alkyl, the alkylene portion thereof can include any of the alkylene groups described herein (e.g., —CH₂—, —CH₂CH₂—, —CH(CH₃)—, —CH₂CH₂CH₂—, —CH(CH₃)CH₂CH₂—, etc. Similarly, the “alkyl” portion of alkylene—O—S(O)₂-alkyl can include any alkyl group described herein (e.g., methyl, ethyl, propyl, butyl, pentyl, etc.) Non-limiting examples of R¹ when R¹ is -alkylene-N(R⁵)₂ include —CH₂—N(R⁵)₂, —CH(CH₃)—N(R⁵)₂, —CH₂CH₂—N(R⁵)₂, —CH₂CH₂CH₂—N(R⁵)₂, —CH(CH₃)CH₂CH₂—N(R⁵)₂, etc., wherein each R⁵ is independently defined as described herein. For example, the “—N(R⁵)₂” portion of -alkylene-N(R⁵)₂ of R¹ can be —NH₂, —N(CH₃)₂, —NH(CH₃), —NH(phenyl), —N(phenyl)₂, —NH—S(O)₂—CH₃, —NH—S(O)₂-cyclopropyl, —NH—C(O)—NH₂, —NH—C(O)—N(CH₃)₂, —NH—C(O)—CH₃, —NH—CH₂CH₂—OH, etc. Non-limiting examples of R¹ when R¹ is -alkylene-OR² include —CH₂—OR², —CH(CH₃)—OR², —CH₂CH₂—OR², —CH(OR²)CH₂CH(CH₃)₂, —CH(CH₃)CH₂CH₂—OR², wherein R² is defined as described herein. For example, the “—OR²” portion of said -alkylene-OR² of R¹ can be —OH, —OCH₃, —OCH₂CH₃, —OCH(CH₃)₂, —O-phenyl. Alternatively, two R¹ groups attached to the same ring carbon atom can form a carbonyl group, for example as shown below:

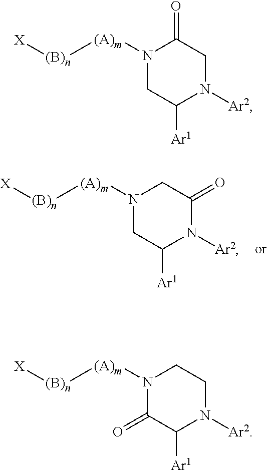

Each R² is independently H, alkyl, or aryl. Non-limiting examples of R² when R² is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R² when R² is aryl include phenyl, naphthyl, etc., wherein said aryl may be unsubstituted or substituted with one or more Y¹ groups as defined herein.

Each R³ is selected from the group consisting of H, alkyl, unsubstituted aryl, aryl substituted with one or more Y¹ groups, —OR², -alkylene-O-alkyl, and -alkylene-OH. Non-limiting examples of R³ when R³ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R³ when R³ is aryl include phenyl, naphthyl, etc., wherein said aryl may be unsubstituted or substituted with one or more Y¹ groups as defined herein. Non-limiting examples of R³ when R³ is —OR² include —OH, —OCH₃, —OCH₂CH₃, —OCH(CH₃)₂, —O-phenyl, etc. Non-limiting examples of R³ when R³ is -alkylene-O-alkyl include —O—CH₂—O—CH₃, —O—CH₂CH₂—O—C(CH₃)₃, —O—CH(CH₃)—O—CH₃, —O—CH₂CH₂—O—CH₃, —O—CH₂CH₂—O—CH₂CH₃, —O—CH(OCH₃)CH₂CH(CH₃)₂, —O—CH(CH₃)CH₂CH₂—O—CH₃, —O—CH₂CH₂—O—CH₂CH₃, etc. Non-limiting examples of R³ when R³ is -alkylene-OH include —CH₂—OH, —CH₂CH₂—OH, —CH₂CH₂CH₂—OH, —CH(OH)CH₃, —CH₂CH(OH)CH₃, etc.

Each R⁴ is selected from the group consisting of H, alkyl, aryl, —C(O)—O-alkyl, —C(O)-alkyl, —C(O)-aryl, and —S(O)₂aryl. Non-limiting examples of R⁴ when R⁴ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁴ when R⁴ is aryl include phenyl, naphthyl, etc., wherein said aryl may be unsubstituted or substituted with one or more Y¹ groups as defined herein. Non-limiting examples of R⁴ when R⁴ is —C(O)—O-alkyl include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH₂CH(CH₃)₂, —C(O)—O—CH(CH₃)CH₂CH₃, —C(O)—O—C(CH₃)₃, —C(O)—O—CH₂CH₂CH₂CH₂CH₃, —C(O)—O—CH₂CH(CH₃)CH₂CH₃, —C(O)—O—CH₂CH₂CH(CH₃)₂, —C(O)—O—CH₂CH₂CH₂CH₂CH₂CH₃, —C(O)—O—CH(CH₃)CH₂CH₂CH₂CH₃, —C(O)—O—CH₂CH(CH₃)CH₂CH₂CH₃, —C(O)—O—CH₂CH₂CH(CH₃)CH₂CH₃, —C(O)—O—CH₂CH₂CH₂CH(CH₃)₂, etc. Non-limiting examples of R⁴ when R⁴ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of R⁴ when R⁴ is —C(O)-aryl include —C(O)-phenyl, —C(O)-naphthyl, etc., optionally substituted with one or more Y¹ groups. Non-limiting examples of R⁴ when R⁴ is —S(O)₂aryl include —S(O)₂-phenyl, —S(O)₂-naphthyl, etc., optionally substituted with one or more Y¹ groups.

Each R⁵ is selected from the group consisting of H, alkyl, aryl, —S(O)₂-alkyl, —S(O)₂-cycloalkyl, —S(O)₂-aryl, —C(O)—N(R²)₂, —C(O)-alkyl, and -alkylene-OH. Non-limiting examples of R⁵ when R⁵ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁵ when R⁵ is aryl include phenyl, naphthyl, etc., wherein said aryl may be unsubstituted or substituted with one or more Z groups as defined herein. Non-limiting examples of R⁵ when R⁵ is —S(O)₂-alkyl include —S(O)₂—CH₃, —S(O)₂—CH₂CH₃, —S(O)₂—CH₂CH₂CH₃, —S(O)₂—CH(CH₃)₂, —S(O)₂—CH₂CH₂CH₂CH₃, —S(O)₂—CH₂CH(CH₃)₂, —S(O)₂—CH(CH₃)CH₂CH₃, —S(O)₂—C(CH₃)₃, —S(O)₂—CH₂CH₂CH₂CH₂CH₃, —S(O)₂—CH₂CH(CH₃)CH₂CH₃, —S(O)₂—CH₂CH₂CH(CH₃)₂, —S(O)₂—CH₂CH₂CH₂CH₂CH₂CH₃, —S(O)₂—CH(CH₃)CH₂CH₂CH₂CH₃, —S(O)₂—CH₂CH(CH₃)CH₂CH₂CH₃, —S(O)₂—CH₂CH₂CH(CH₃)CH₂CH₃, —S(O)₂—CH₂CH₂CH₂CH(CH₃)₂, etc. Non-limiting examples of R⁵ when R⁵ is —S(O)₂-cycloalkyl include —S(O)₂-cyclopropyl, —S(O)₂-cyclobutyl, —S(O)₂-cyclopentyl, —S(O)₂-cyclohexyl, —S(O)₂-adamantyl, —S(O)₂-norbornyl, —S(O)₂-decanyl, etc. Non-limiting examples of R⁵ when R⁵ is —C(O)—N(R²)₂ include —C(O)—NH₂, —C(O)—NH(alkyl), —C(O)—N(alkyl)₂, —C(O)—NH(aryl), —C(O)—N(alkyl)(aryl), —C(O)—N(aryl)₂, wherein the terms “aryl” and “alkyl” are as defined above, and said “aryl” may be unsubstituted or substituted with one or more Y¹ groups as defined herein. Non-limiting examples of R⁵ when R⁵ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of R⁵ when R⁵ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of R⁵ when R⁵ is —S(O)₂aryl include —S(O)₂-phenyl, —S(O)₂-naphthyl, etc., optionally substituted with one or more Y¹ groups.

Each Y¹ is independently selected from the group consisting of alkyl, cycloalkyl, heterocycloalkyl, heterocycloalkenyl, halo, haloalkyl, benzyl, aryl, heteroaryl, —O-aryl, —S-aryl, —S(O)₂-alkyl, —S(O)₂-cycloalkyl, —S(O)₂-aryl, -alkylene-CN, —CN, —C(O)-alkyl, —C(O)-aryl, —C(O)-haloalkyl, —C(O)O-alkyl, —N(R²)C(O)-alkyl, —N(R²)C(O)—N(R²)₂, —OH, —O-alkyl, —O-haloalkyl, —O-alkylene-C(O)OH, —S-alkyl, —S-haloalkyl, -alkylene-OH, -alkylene-C(O)—O-alkyl, —O-alkylene-aryl, and —N(R⁵)₂. Non-limiting examples of Y¹ when Y¹ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of Y¹ when Y¹ is cycloalkyl include cyclopropyl, cyclobutyl, cyclopentyl, cyclohexyl, adamantyl, norbornyl, etc. Non-limiting examples of Y¹ when Y¹ is heterocycloalkyl include morpholinyl, piperazinyl, piperidinyl, pyrrolidinyl, tetrahydrofuranyl, tetrahydrothiophenyl, tetrahydropyranyl, azetidinyl, etc. Non-limiting examples of Y¹ when Y¹ is heterocycloalkenyl include 2H-benzo[1,4]oxazinyl, 4H-chromenyl, 4H-chromenyl, 3H-indolyl, 1H-isoindolyl, 4H-benzo[1,4]oxazinyl, etc. Non-limiting examples of Y¹ when Y¹ is halo include chloro, bromo, and iodo. Non-limiting examples of Y¹ when Y¹ is haloalkyl include —CF₃, —CHF₂, —CH₂F, —CH₂CF₃, —CF₂CF₃, —CH₂Br, —CH₂Cl, —CCl₃, etc. Non-limiting examples of Y¹ when Y¹ is aryl include phenyl, naphthyl, etc. Non-limiting examples of Y¹ when Y¹ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of Y¹ when Y¹ is —O-aryl include —O-phenyl, —O-naphthyl, etc. Non-limiting examples of Y¹ when Y¹ is —S-aryl include —S-phenyl, —S-naphthyl, etc. Non-limiting examples of Y¹ when Y¹ is —S(O)₂-alkyl include —S(O)₂—CH₃, —S(O)₂—CH₂CH₃, —S(O)₂—CH₂CH₂CH₃, —S(O)₂—CH(CH₃)₂, —S(O)₂—CH₂CH₂CH₂CH₃, —S(O)₂—CH₂CH(CH₃)₂, —S(O)₂—CH(CH₃)CH₂CH₃, —S(O)₂—C(CH₃)₃, —S(O)₂—CH₂CH₂CH₂CH₂CH₃, —S(O)₂—CH₂CH(CH₃)CH₂CH₃, —S(O)₂—CH₂CH₂CH(CH₃)₂, —S(O)₂—CH₂CH₂CH₂CH₂CH₂CH₃, —S(O)₂—CH(CH₃)CH₂CH₂CH₂CH₃, —S(O)₂—CH₂CH(CH₃)CH₂CH₂CH₃, —S(O)₂—CH₂CH₂CH(CH₃)CH₂CH₃, —S(O)₂—CH₂CH₂CH₂CH(CH₃)₂, etc. Non-limiting examples of Y¹ when Y¹ is —S(O)₂-cycloalkyl include —S(O)₂-cyclopropyl, —S(O)₂-cyclobutyl, —S(O)₂-cyclopentyl, —S(O)₂-cyclohexyl, —S(O)₂-adamantyl, —S(O)₂-norbornyl, etc. Non-limiting examples of Y¹ when Y¹ is —S(O)₂-aryl include —S(O)₂-phenyl, —S(O)₂-naphthyl, etc. Non-limiting examples of Y¹ when Y¹ is -alkylene-CN include —O—CH₂—CN, —O—CH₂CH₂—CN, —CH₂CH₂CH₂CN, —O—CH(CH₃)—CN, —O—CH(CN)CH₂CH(CH₃)₂, —O—CH(CH₃)CH₂CH₂—CN, etc. Non-limiting examples of Y¹ when Y¹ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of Y¹ when Y¹ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of Y¹ when Y¹ is —C(O)-aryl include —C(O)-phenyl, —C(O)-naphthyl, etc. Non-limiting examples of Y¹ when Y¹ is —C(O)-haloalkyl include —C(O)—CF₃, —C(O)—CHF₂, —C(O)—CH₂F, —C(O)—CH₂CF₃, —C(O)—CF₂CF₃, —C(O)—CH₂Br, —C(O)—CH₂Cl, —C(O)—CCl₃, etc. Non-limiting examples of Y¹ when Y¹ is —C(O)O-alkyl include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH₂CH(CH₃)₂, —C(O)—O—CH(CH₃)CH₂CH₃, —C(O)—O—C(CH₃)₃, —C(O)—O—CH₂CH₂CH₂CH₂CH₃, —C(O)—O—CH₂CH(CH₃)CH₂CH₃, —C(O)—O—CH₂CH₂CH(CH₃)₂, —C(O)—O—CH₂CH₂CH₂CH₂CH₂CH₃, —C(O)—O—CH(CH₃)CH₂CH₂CH₂CH₃, —C(O)—O—CH₂CH(CH₃)CH₂CH₂CH₃, —C(O)—O—CH₂CH₂CH(CH₃)CH₂CH₃, —C(O)—O—CH₂CH₂CH₂CH(CH₃)₂, etc. Non-limiting examples of Y¹ when Y¹ is —N(R²)C(O)-alkyl include —NH—C(O)-alkyl, —N(alkyl)-C(O)-alkyl, and —N(aryl)-C(O)-alkyl wherein the terms “alkyl” and “aryl” are as defined above. Non-limiting examples of Y¹ when Y¹ is —N(R²)C(O)—N(R²)₂ include —NHC(O)—NH₂, —NHC(O)—N(alkyl)₂, —NHC(O)—N(aryl)₂, —NHC(O)—NH-alkyl, —NHC(O)—NH-aryl, —N(alkyl)C(O)—NH-alkyl, —N(alkyl)C(O)—NH-aryl, —N(aryl)C(O)—NH-aryl, —N(aryl)C(O)—NH-aryl, etc. Non-limiting examples of Y¹ when Y¹ is —O-alkyl include —O—CH₃, —O—CH₂CH₃, —O—CH₂CH₂CH₃, —O—CH(CH₃)₂, —O—CH₂CH₂CH₂CH₃, —O—CH₂CH(CH₃)₂, —O—CH(CH₃)CH₂CH₃, —O—C(CH₃)₃, —O—CH₂CH₂CH₂CH₂CH₃, —O—CH₂CH(CH₃)CH₂CH₃, —O—CH₂CH₂CH(CH₃)₂, —O—CH₂CH₂CH₂CH₂CH₂CH₃, —O—CH(CH₃)CH₂CH₂CH₂CH₃, —O—CH₂CH(CH₃)CH₂CH₂CH₃, —O—CH₂CH₂CH(CH₃)CH₂CH₃, —O—CH₂CH₂CH₂CH(CH₃)₂, etc. Non-limiting examples of Y¹ when Y¹ is —O-haloalkyl include —O—CF₃, —O—CHF₂, —O—CH₂F, —O—CH₂CF₃, —O—CF₂CF₃, —O—CH₂Br, —O—CH₂Cl, —O—CCl₃, etc. Non-limiting examples of Y¹ when Y¹ is —O-alkylene-C(O)OH include —O—CH₂—C(O)OH, —O—CH₂CH₂—C(O)OH, —CH₂OH₂OH₂O(O)OH, —O—CH(CH₃)—C(O)OH, —O—CH(C(O)OH)CH₂CH(CH₃)₂, —O—CH(CH₃)CH₂CH₂—C(O)OH, etc. Non-limiting examples of Y¹ when Y¹ is —S-alkyl include —S—CH₃, —S—CH₂CH₃, —S—CH₂CH₂CH₃, —S—CH(CH₃)₂, —S—CH₂CH₂CH₂CH₃, —S—CH₂CH(CH₃)₂, —S—CH(CH₃)CH₂CH₃, —S—C(CH₃)₃, —S—CH₂CH₂CH₂CH₂CH₃, —S—CH₂CH(CH₃)CH₂CH₃, —S—CH₂CH₂CH(CH₃)₂, —S—CH₂CH₂CH₂CH₂CH₂CH₃, —S—CH(CH₃)CH₂CH₂CH₂CH₃, —S—CH₂CH(CH₃)CH₂CH₂CH₃, —S—CH₂CH₂CH(CH₃)CH₂CH₃, —S—CH₂CH₂CH₂CH(CH₃)₂, etc. Non-limiting examples of Y¹ when Y¹ is —S-haloalkyl include —S—CF₃, —S—CHF₂, —S—CH₂F, —S—CH₂CF₃, —S—CF₂CF₃, —S—CH₂Br, —S—CH₂Cl, —S—CCl₃, etc. Non-limiting examples of Y¹ when Y¹ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of Y¹ when Y¹ is -alkylene-C(O)—O-alkyl include —O—CH₂—C(O)O—CH₃, —O—CH₂—C(O)O—CH₂CH₃, —O—CH₂CH₂—C(O)O—CH₂CH₃, —O—CH₂CH₂CH₂—C(O)O—CH₃, —O—CH₂CH₂—O(O)O—C(CH₃)₃, —O'CH(CH₃)—O(O)O—OH₃, —O—CH₂CH₂—O(O)O—OH₃, —O—CH(C(O)OCH₃)CH₂CH(CH₃)₂, —O—CH(CH₃)CH₂CH₂—C(O)O—CH₃, etc. Non-limiting examples of Y¹ when Y¹ is —O-alkylene-aryl include —O—CH₂-phenyl, —O—CH₂CH₂-phenyl, —O—CH(CH₃)-phenyl, —O—CH₂CH(CH₃)-phenyl, —OC(CH₃)₂-phenyl, —O—CH(CH₂CH₃)-phenyl, etc. Non-limiting examples of Y¹ when Y¹ is —N(R⁵)₂ include —NH₂, —N(CH₃)₂, —NH(CH₃), —NH(phenyl), —N(phenyl)₂, —NH—S(O)₂—CH₃, —NH—S(O)₂-cyclopropyl, —NH—C(O)—NH₂, —NH—C(O)—N(CH₃)₂, —NH—C(O)—CH₃, —NH—CH₂CH₂—OH, etc. The aryl or heteroaryl portions of any of the groups of Y¹ may be unsubstituted or substituted with one or more Z groups as defined herein.

Each Y² is independently selected from the group consisting of alkyl, haloalkyl, aryl, -alkylene-aryl, —CN, —C(O)-alkyl, —S(O)₂-cycloalkyl, -alkylene-N(R²)₂, —C(O)-alkylene-N(R⁴)₂, —C(O)—O-alkyl, —C(O)-aryl, and —C(O)-haloalkyl. Non-limiting examples of Y² when Y² is alkyl include —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —(CH₃)₃, —CH₂CH₂CH₂CH₂CH₃, —CH₂CH(CH₃)CH₂CH₃, —CH₂CH₂CH(CH₃)₂, —CH₂CH₂CH₂CH₂CH₂CH₃, —CH(CH₃)CH₂CH₂CH₂CH₃, —CH₂CH(CH₃)CH₂CH₂CH₃, —CH₂CH₂CH(CH₃)CH₂CH₃, —CH₂CH₂CH₂CH(CH₃)₂, etc. Non-limiting examples of Y² when Y² is aryl include phenyl, naphthyl, etc. Non-limiting examples of Y² when Y² is -alkylene-aryl include —CH₂-phenyl, —CH₂CH₂-phenyl, —CH(CH₃)-phenyl, —CH₂CH(CH₃)-phenyl, —C(CH₃)₂-phenyl, —CH(CH₂CH₃)-phenyl, etc. Non-limiting examples of Y² when Y² is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of Y² when Y² is —S(O)₂-cycloalkyl include —S(O)₂-cyclopropyl, —S(O)₂-cyclobutyl, —S(O)₂-cyclopentyl, —S(O)₂-cyclohexyl, —S(O)₂-norbornyl, —S(O)₂-adamantyl, etc. Non-limiting examples of Y² when Y² is -alkylene-N(R²)₂ include -alkylene-N(R²)₂ include —CH₂—N(R²)₂, —CH(CH₃)—N(R²)₂, —CH₂CH₂—N(R²)₂, —CH₂CH₂CH₂—N(R²)₂, —CH(CH₃)CH₂CH₂—N(R²)₂, etc., wherein each R² is independently defined as described herein. For example, the “—N(R²)₂” portion of -alkylene-N(R²)₂ of Y² can be —NH₂, —N(CH₃)₂, —NH(CH₃), —NH(phenyl), —N(phenyl)₂, —N(CH₂CH₃)₂, —NH(CH₂CH₃), etc. Non-limiting examples of Y² when Y² is —C(O)-alkylene-N(R⁴)₂ include —C(O)—CH₂—-N(R⁴)₂, —C(O)—CH(CH₃)—N(R⁴)₂, —C(O)—CH₂CH₂—N(R⁴)₂, —C(O)—CH₂CH₂CH₂—N(R⁴)₂, —C(O)—CH(CH₃)CH₂CH₂—N(R⁴)₂, etc., wherein each R⁴ is independently defined as described herein. For example the “—N(R⁴)₂” portion of —C(O)-alkylene-N(R⁴)₂ of Y² can be —NH₂, —N(CH₃)₂, —NH(CH₃), —NH(phenyl), —N(phenyl)₂, —N(CH₂CH₃)₂, —NH(CH₂CH₃), —NH—C(O)—O—CH₃, —NH—C(O)—O—CH₂CH₃, —N(CH₃)—C(O)—O—CH₃, —N(CH₃)—C(O)—O—CH₂CH₃, —NH—C(O)—CH₃, —NH—C(O)—CH₂CH₃, —N(CH₃)—C(O)—CH₃, —N(CH₃)—C(O)—CH₂CH₃, etc. Non-limiting examples of Y² when Y² is —C(O)—O-alkyl include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH₂CH(CH₃)₂, —C(O)—O—CH(CH₃)CH₂CH₃, —C(O)—O—C(CH₃)₃, —C(O)—O—CH₂CH₂CH₂CH₂CH₃, —C(O)—O—CH₂CH(CH₃)CH₂CH₃, —C(O)—O—CH₂CH₂CH(CH₃)₂, —C(O)—O—CH₂CH₂CH₂CH₂CH₂CH₃, —C(O)—O—CH(CH₃)CH₂CH₂CH₂CH₃, —C(O)—O—CH₂CH(CH₃)CH₂CH₂CH₃, —C(O)—O—CH₂CH₂CH(CH₃)CH₂CH₃, —C(O)—O—CH₂CH₂CH₂CH(CH₃)₂, etc. Non-limiting examples of Y² when Y² is —C(O)-aryl include —C(O)-phenyl, —C(O)-naphthyl, etc., optionally substituted with one or more Z groups. Non-limiting examples of Y² when Y² is —C(O)-haloalkyl include —C(O)—CF₃, —C(O)—CHF₂, —C(O)—CH₂F, —C(O)—CH₂CF₃, —C(O)—CF₂CF₃, —C(O)—CH₂Br, —C(O)—CH₂Cl, —C(O)—CCl₃, etc.

Each Z is independently selected from the group consisting of alkyl, halo, haloalkyl, —OH, —O-alkyl, and —CN. The terms “alkyl”, “halo”, aloalkyl”, and “—O-alkyl” are as defined above.

As used throughout the specification, the following terms, unless otherwise indicated, shall be understood to have the following meanings:

In each of the various embodiments of the compounds of the invention described herein, including the compounds of the examples, e.g., those of Table LIV, such formulas and examples are intended to encompass all forms of the compounds such as, for example, any solvates, hydrates, stereoisomers, and tautomers of said compounds, and of any pharmaceutically acceptable salts of such compounds, stereoisomers, solvates, hydrates, and tautomers thereof. Such compounds are intended to encompass the neat chemical, e.g., in isolated and/or purified form.

The term “Patient” includes humans and/or other animals. Animals include mammals and non-mammalian animals. Mammals include humans and other mammalian animals. In some embodiments, the patient is a human. In other embodiments, the patient is non-human. In some embodiments, non-human animals include companion animals. Examples of companion animals include house cats (feline), dogs (canine), rabbits, horses (equine), guinea pigs, rodents (e.g., rats, mice, gerbils, or hamsters), primates (e.g., monkeys), and avians (e.g., pigeons, doves, parrots, parakeets, macaws, or canaries). In some embodiments, the animals are felines (e.g., house cats). In some embodiments, the animals are canines. Canines include, for example, wild and zoo canines, such as wolves, coyotes, and foxes. Canines also include dogs, particularly domestic dogs, such as, for example, pure-bred and/or mongrel companion dogs, show dogs, working dogs, herding dogs, hunting dogs, guard dogs, police dogs, racing dogs, and/or laboratory dogs. In some embodiments, non-human animals include wild animals; livestock animals (e.g., animals raised for food and/or other products, such as, for example, meat, poultry, fish, milk, butter, eggs, fur, leather, feathers, and/or wool); beasts of burden; research animals; companion animals; and animals raised for/in zoos, wild habitats, and/or circuses. In other embodiments, non-human animals include primates, such as monkeys and great apes. In other embodiments, animals include bovine (e.g., cattle or dairy cows), porcine (e.g., hogs or pigs), ovine (e.g., goats or sheep), equine (e.g., horses), canine (e.g., dogs), feline (e.g., house cats), camels, deer, antelope, rabbits, guinea pigs, rodents (e.g., squirrels, rats, mice, gerbils, or hamsters), cetaceans (e.g., whales, dolphins, or porpoises), pinnipeds (e.g., seals or walruses). In other embodiments, animals include avians. Avians include birds associated with either commercial or noncommercial aviculture. These include, for example, Anatidae, such as swans, geese, and ducks; Columbidae, such as doves and pigeons (e.g., such as domestic pigeons); Phasianidae, such as partridges, grouse and turkeys; Thesienidae, such as domestic chickens; Psittacines, such as parakeets, macaws, and parrots (e.g., parakeets, macaws, and parrots raised for pets or collector markets; game birds; and ratites, such as ostriches. In other embodiments, animals include fish. Fish include, for example, the Teleosti grouping of fish (i.e., teleosts), such as, for example, the Salmoniformes order (which includes the Salmonidae family) and the Perciformes order (which includes the Centrarchidae family). Examples of fish include the Salmonidae family, the Serranidae family, the Sparidae family, the Cichlidae family, the Centrarchidae family, the three-Line Grunt (Parapristipoma trilineatum), and the Blue-Eyed Plecostomus (Plecostomus spp). Additional examples of fish include, for example, catfish, sea bass, tuna, halibut, arctic charr, sturgeon, turbot, flounder, sole, carp, tilapia, striped bass, eel, sea bream, yellowtail, amberjack, grouper, and milkfish. In other embodiments, animals include marsupials (e.g., kangaroos), reptiles (e.g., farmed turtles), amphibians (e.g., farmed frogs), crustaceans (e.g., lobsters, crabs, shrimp, or prawns), mollusks (e.g., octopus and shellfish), and other economically-important animals.

“Body Condition Score” refers to an assessment of an animal's weight for age and weight for height ratios, and its relative proportions of muscle and fat. The assessment is made by eye, on the basis of amount of tissue cover between the points of the hip, over the transverse processes of the lumbar vertebrae, the cover over the ribs and the pin bones below the tail. Each animal is graded by comparison with animals pictured on a chart. The grading may be expressed as a score ranging from 1 to 8. As used herein, Body Condition Scores of 1 to 8 are described as follows:

| Score | Description |
|---|---|
| 1 | Emaciated. Ribs, lumbar vertebrae, pelvic bones |
| | and all bony prominences evident from a distance. |
| | No discernable body fat. Obvious loss of muscle |
| | mass. |
| 2 | Very thin. Ribs, lumbar vertebrae and pelvic bond |
| | easily visible. No palpable fat. Some evidence of |
| | other bony prominence. Minimal loss of muscle |
| | mass. |
| 3 | Thin. Ribs easily palpated and may be visible with |
| | no palpable fat. Tops of lumbar vertebrae visible. |
| | Pelvic bones becoming prominent. Obvious waist |
| | and lack of abdominal tuck. |
| 4 | Underweight. Ribs easily palpable with minimal fat |
| | covering. Waist easily noted from above. Abdominal |
| | tuck evident. |
| 5 | Ideal. Ribs palpable without excess fat covering. |
| | Waist observed behind ribs when viewed from above. |
| | Abdomen tucked when viewed from the side. |
| 6 | Overweight. Ribs palpable with slight excess fat |
| | covering. Waist is discernable viewed from above, |
| | but is not prominent. Abdominal tuck apparent. |
| 7 | Heavy. Ribs palpable with difficulty, heavy fat cover. |
| | Noticeable fat deposits over lumbar area and base of |
| | tail. Waist absent or barely visible. Abdominal tuck |
| | may be present. |
| 8 | Obese. Ribs not palpable under very heavy fat |
| | cover, or palpable only with significant pressure. |
| | Heavy fat deposits over lumbar area and base of tail. |
| | Waist absent. NO abdominal tuck. Obvious |
| | abdominal distension may be present. |

“Alkyl” means an aliphatic hydrocarbon group which may be straight or branched and comprising about 1 to about 20 carbon atoms in the chain. In one embodiment alkyl groups contain about 1 to about 12 carbon atoms in the chain. In another embodiment alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl, n-butyl, t-butyl, n-pentyl, heptyl, nonyl, or decyl.

“Alkylene” means a divalent group obtained by removal of a hydrogen atom from an alkyl group that is defined above. Non-limiting examples of alkylene include methylene, ethylene and propylene. “Lower alkylene” means an alkylene having about 1 to 6 carbon atoms in the chain, which may be straight or branched.

“Alkenyl” means an aliphatic hydrocarbon group containing at least one carbon-carbon double bond and which may be straight or branched and comprising about 2 to about 15 carbon atoms in the chain. In one embodiment alkenyl groups have about 2 to about 12 carbon atoms in the chain. In another embodiment alkenyl groups have about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkenylene” means a divalent group obtained by removal of a hydrogen atom from an alkenyl group that is defined above.

“Alkynyl” means an aliphatic hydrocarbon group containing at least one carbon-carbon triple bond and which may be straight or branched and comprising about 2 to about 15 carbon atoms in the chain. In one embodiment alkynyl groups have about 2 to about 12 carbon atoms in the chain. In another embodiment alkynyl groups have about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The term “substituted alkynyl” means that the alkynyl group may be substituted by one or more substituents which may be the same or different, each substituent being independently selected from the group consisting of alkyl, aryl and cycloalkyl.

“Aryl” (sometimes abbreviated “ar” or “Ar”) means an aromatic monocyclic or multicyclic ring system comprising about 6 to about 14 carbon atoms, or about 6 to about 10 carbon atoms. The aryl group can be optionally substituted with one or more “ring system substituents” which may be the same or different, and are as defined herein. Non-limiting examples of suitable aryl groups include phenyl, naphthyl, and biphenyl.

“Aryloxy” means a —O-aryl group, wherein aryl is defined as above. the aryloxy group is attached to the parent moiety through the ether oxygen.

“Arylene” means a divalent aryl group obtained by the removal of a hydrogen atom from an aryl group as defined above. Non-limiting examples of arylenes include, for example, 1,2-phenylene, 1,3-phenylene, or 1,4-phenylene.

“Heteroaryl” means an aromatic monocyclic or multicyclic ring system comprising about 5 to about 14 ring atoms, or about 5 to about 10 ring atoms, in which one or more of the ring atoms is an element other than carbon, for example nitrogen, oxygen or sulfur, alone or in combination. In one embodiment heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Cycloalkyl” means a non-aromatic mono- or multicyclic ring system comprising about 3 to about 13 carbon atoms, or about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like.

“Cycloalkylene” means a divalent cycloalkyl group obtained by the removal of a hydrogen atom from a cycloalkyl group as defined above. Non-limiting examples of cycloalkylenes include:

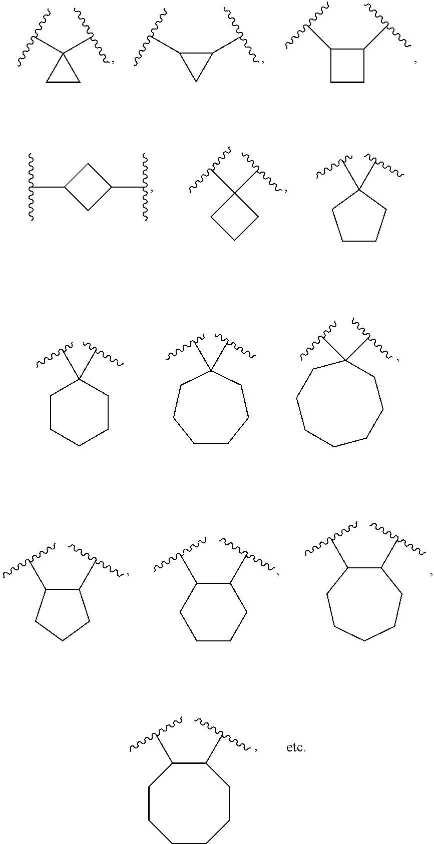

“Heterocycloalkyl” means a non-aromatic saturated monocyclic or multicyclic ring system comprising about 3 to about 10 ring atoms, or about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. In one embodiment heterocycloalkyls contain about 5 to about 6 ring atoms. The prefix aza, oxa or thia before the heterocycloalkyl root name means that at least a nitrogen, oxygen or sulfur atom respectively is present as a ring atom. The heterocycloalkyl can be optionally substituted by one or more “ring system substituents” which may be the same or different, and are as defined herein. The nitrogen or sulfur atom of the heterocycloalkyl can be optionally oxidized to the corresponding N-oxide, S-oxide or S,S-dioxide. Non-limiting examples of suitable monocyclic heterocycloalkyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heterocycloalkenyl” means a non-aromatic unsaturated monocyclic or multicyclic ring system comprising about 3 to about 10 ring atoms, or about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Heterocycloalkenyls have at least one double bond, wherein said double bond may be between two ring carbon atoms, between a ring carbon atom and a ring heteroatom (e.g., between a ring carbon atom and a ring nitrogen atom), or between two ring heteroatoms (e.g., between two ring nitrogen atoms). If more than one double bond is present in the ring, each double bond is independently defined as described herein. In another embodiment heterocycloalkenyls contain about 5 to about 6 ring atoms. The prefix aza, oxa or thia before the heterocycloalkenyl root name means that at least a nitrogen, oxygen or sulfur atom respectively is present as a ring atom. The heterocycloalkenyl can be optionally substituted by one or more “ring system substituents” which may be the same or different, and are as defined herein. The nitrogen or sulfur atom of the heterocycloalkenyl can be optionally oxidized to the corresponding N-oxide, S-oxide or S,S-dioxide. Non-limiting examples of suitable monocyclic heterocycloalkenyl rings include thiazolinyl, 2,3-dihydro-1H-pyrrolyl, 2,5-dihydro-1H-pyrrolyl, 3,4-dihydro-2H-pyrrolyl, 2,3-dihydro-furan, 2,5-dihydro-furan, etc.

“Benzo-fused heterocycloalkenyl” means a heterocycloalkenyl, as defined above, to which one or more phenyl rings has been fused, so that each phenyl ring shares two ring carbon atoms with the cycloalkyl ring. Non-limiting examples of benzo-fused cycloalkyls are 4H-chromene, chromene-4-one, 1H-isochromene, etc.

“Benzo-fused cycloalkyl” means a cycloalkyl, as defined above, to which one or more phenyl rings has been fused, so that each phenyl ring shares two ring carbon atoms with the cycloalkyl ring. Non-limiting examples of benzo-fused cycloalkyls are indanyl and tetradehydronaphthyl:

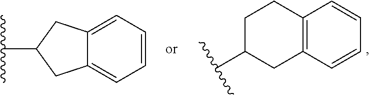 [SMILES: *C1Cc2ccccc2C1 ; *C1CCc2ccccc2C1]

and non-limiting examples of a dibenzo-fused cycloalkyls are fluorenyl:

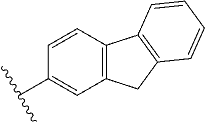 [SMILES: *c1ccc2c(c1)Cc1ccccc1-2]

and acenaphthenyl:

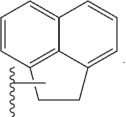

“Benzo-fused heterocycloalkyl” means a heterocycloalkyl, as defined above, to which one or more phenyl rings has been fused, so that each phenyl ring shares two ring carbon atoms with the heterocycloalkyl ring. A non-limiting example of a benzo-fused heterocycloalkyls is 2,3-dihydro-benzo[1,4]dioxinyl.

“Cycloalkenyl” means a non-aromatic mono or multicyclic ring system comprising about 3 to about 10 carbon atoms, or about 5 to about 10 carbon atoms, which contains at least one carbon-carbon double bond. In one embodiment cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Halo” (or “halogeno” or “halogen”) means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl are replaced by a halo group as defined above.

“Ring system substituent” means a substituent attached to an aromatic or non-aromatic ring system which, for example, replaces an available hydrogen on the ring system. Ring system substituents may be the same or different, and are defined as described herein.

“Alkoxy” means an —O-alkyl group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

When used herein, the term “independently”, in reference to the substitution of a parent moiety with one or more substituents, means that the parent moiety may be substituted with any of the listed substituents, either individually or in combination, and any number of chemically possible substituents may be used. As a non-limiting example, a phenyl independently substituted with one or more alkyl or halo substituents can include, chlorophenyl, dichlorophenyl, trichlorophenyl, tolyl, xylyl, 2-chloro-3-methylphenyl, 2,3-dichloro-4-methylphenyl, etc.

As used herein, the term “composition” is intended to encompass a product comprising the specified ingredients in the specified amounts, as well as any product which results, directly or indirectly, from combination of the specified ingredients in the specified amounts.

The wavy line [symbol] as a bond generally indicates a mixture of, or either of, the possible isomers, e.g., containing (R)- and (S)— stereochemistry. For example,

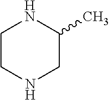

means containing both

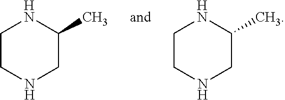

Moreover, when the stereochemistry of a chiral center (or stereogenic center) is not expressly indicated, a mixture of, or any of the individual possible isomers are contemplated. Thus, for example,

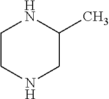

means containing

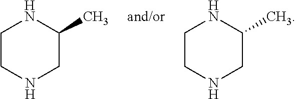

Lines drawn into the ring systems, such as, for example:

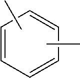

indicate that the indicated line (bond) may be attached to any of the substitutable ring carbon atoms.

As well known in the art, a bond drawn from a particular atom wherein no moiety is depicted at the terminal end of the bond indicates a methyl group bound through that bond to the atom, unless stated otherwise. For example:

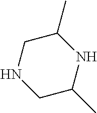

represents

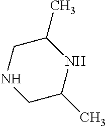

It should also be noted that any carbon or heteroatom with unsatisfied valences in the text, schemes, examples, structural formulae, and any Tables herein is assumed to have the hydrogen atom or atoms to satisfy the valences.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in any Formula (e.g., Formula I), its definition on each occurrence is independent of its definition at every other occurrence.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the present invention may also exist as, or optionally be converted to a solvate. The preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The compounds of Formula (I) form salts that are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a piperazine, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of Formula (I), and salts, solvates and prodrugs thereof, may exist in their tautomeric form (for example, as an amide or imino ether). All such tautomeric forms are contemplated herein as part of the present invention.

The compounds of the present invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, those of ordinary skill in the art will recognize any compounds of the present invention that may be atropisomers (e.g., substituted biaryls). Such atropisomers are considered as part of this invention. Enantiomers can also be separated by use of chiral HPLC column.

Compounds of Formula (I), and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates and prodrugs of the compounds of Formula (I), are intended to be included in the present invention.

In still another embodiment, the present invention provides a composition comprising at least one compound of Formula (I), or a pharmaceutically acceptable salt, solvate, or ester thereof, and a pharmaceutically acceptable carrier.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Unit dosage forms, without limitation, can include tablets, pills, capsules, sustained release pills, sustained release tablets, sustained release capsules, powders, granules, or in the form of solutions or mixtures (i.e., elixirs, tinctures, syrups, emulsions, suspensions). For example, one or more compounds of Formula (I), or salts or solvates thereof, may be combined, without limitation, with one or more pharmaceutically acceptable liquid carriers such as ethanol, glycerol, or water, and/or one or more solid binders such as, for example, starch, gelatin, natural sugars (e.g., glucose or δ-lactose), and/or natural or synthetic gums (e.g., acacia, tragacanth, or sodium alginate), carboxymethylcellulose, polyethylene glycol, waxes and the like, and/or disintegrants, buffers, preservatives, anti-oxidants, lubricants, flavorings, thickeners, coloring agents, emulsifiers and the like. In addition, the unit dosage forms can include, without limitation, pharmaceutically acceptable lubricants (e.g., sodium oleate, sodium stearate, magnesium stearate, sodium benzoate, sodium acetate, and sodium chloride) and disintegrators (e.g., starch, methyl cellulose, agar, bentonite, and xanthan gum).

The amount of excipient or additive can range from about 0.1 to about 90 weight percent of the total weight of the treatment composition or therapeutic combination. One skilled in the art would understand that the amount of carrier(s), excipients and additives (if present) can vary.

In another embodiment, the present invention provides a method of treating, reducing, or ameliorating a disease or condition selected from the group consisting of metabolic syndrome, obesity, waist circumference, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of at least one compound of Formula (I), or a pharmaceutically acceptable salt, solvate, or ester thereof.

In yet another embodiment, the present invention provides a method of treating, reducing, or ameliorating obesity, in a patient in need thereof, comprising administering to said patient an effective amount of at least one compound of Formula (I), or a pharmaceutically acceptable salt, solvate, or ester thereof.

In yet another embodiment, the present invention provides a method of treating, reducing, or ameliorating metabolic syndrome, obesity, waist circumference, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of a composition comprising at least one compound of Formula (I), or a pharmaceutically acceptable salt, solvate, or ester thereof and a pharmaceutically acceptable carrier.

In yet another embodiment, the present invention provides a method of treating, reducing, or ameliorating obesity, in a patient in need thereof, comprising administering to said patient an effective amount of a composition comprising at least one compound of Formula (I), or a pharmaceutically acceptable salt, solvate, or ester thereof and a pharmaceutically acceptable carrier.

In another embodiment, the present invention provides a method of treating, reducing, or ameliorating a disease or condition selected from psychic disorders, anxiety, schizophrenia, depression, abuse of psychotropes, abuse and/or dependence of a substance, alcohol dependency, nicotine dependency, neuropathies, migraine, stress, epilepsy, dyskinesias, Parkinson's disease, amnesia, senile dementia, Alzheimer's disease, eating disorders, diabetes type II or non insulin dependent diabetes (NIDD), gastrointestinal diseases, vomiting, diarrhea, urinary disorders, infertility disorders, inflammations, infections, cancer, neuroinflammation, in particular in atherosclerosis, or the Guillain-Barr syndrome, viral encephalitis, cerebral vascular incidents and cranial trauma.

In yet another embodiment, the present invention provides a method of treating, reducing, or ameliorating obesity, in a patient in need thereof, comprising administering to said patient an effective amount of at least one compound of Formula (I), or a pharmaceutically acceptable salt, solvate, or ester thereof.

In another embodiment, the present invention provides a method of treating, reducing, or ameliorating metabolic syndrome, obesity, waist circumference, abdominal girth, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of a composition comprising at least one compound of Formula (I), or a pharmaceutically acceptable salt, solvate, or ester thereof and a pharmaceutically acceptable carrier.

In another embodiment, the present invention provides a method of treating, reducing, or ameliorating hepatic lipidosis and/or fatty liver disease (including but not limited to non-alcoholic fatty liver disease) in a patient in need thereof, comprising administering to said patient an effective amount of a composition comprising at least one compound of Formula (I), or a pharmaceutically acceptable salt, solvate, or ester thereof and a pharmaceutically acceptable carrier.

In another embodiment, the present invention provides a method of reducing body condition score (BCS) in a patient in need thereof, comprising administering to said patient an effective amount of a composition comprising at least one compound of Formula (I), or a pharmaceutically acceptable salt, solvate, or ester thereof (optionally together with at least one additional active agent) and one or more pharmaceutically acceptable carriers. In one embodiment, BCS is reduced from obese to ideal. In another embodiment, BCS is reduced from obese to heavy, overweight, or ideal. In another embodiment, BCS is reduced from obese to heavy. In another embodiment, BCS is reduced from obese to overweight. In another embodiment, BCS is reduced from heavy to overweight or ideal. In another embodiment, BCS is reduced from heavy to ideal. In another embodiment, BCS is reduced from overweight to ideal.

In other embodiments, the present invention provides a method of reducing the abdominal girth in a patient in need thereof. The method comprises administering an effective amount of a composition comprising at least one compound of Formula (I), or a pharmaceutically acceptable salt, solvate, or ester thereof (optionally together with at least one additional active agent) and one or more pharmaceutically acceptable carriers. In some embodiments, the patient is a non-human animal. In some such embodiments, for example, the patient may be a companion mammal, such as a dog, cat, or horse. Girth measurements are taken at the widest point behind the last rib and in front of the pelvis.

In other embodiments, the present invention provides a method of repartitioning, wherein energy of an animal is partitioned away from fat deposition toward protein accretion. The method comprising administering to said patient an effective amount of a composition comprising at least one compound of Formula (I), or a pharmaceutically acceptable salt, solvate, or ester thereof (optionally together with at least one additional active agent) and one or more pharmaceutically acceptable carriers. In some embodiments, the patient is a non-human animal. In some such embodiments, for example, the patient may be a food animal, such as a bovine animal, swine animal, sheep, goat, or poultry animal (chicken, turkey, etc.). In other embodiments, the animal is an equine animal.

In other embodiments, the present invention provides a method of treating, reducing, or ameliorating a disease or condition selected from the group consisting of metabolic syndrome, obesity, waist circumference, abdominal girth, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of at least one compound of Formula (I), or a pharmaceutically acceptable salt, solvate, isomer, or ester thereof.

The compounds of Formula (I) can be useful as CB₁ receptor antagonists for treating, reducing, or ameliorating metabolic syndrome, obesity, waist circumference, lipid profile, insulin sensitivity, neuroinflammatory disorders, cognitive disorders, psychosis, addictive behavior (e.g., smoking cessation), gastrointestinal disorders, and cardiovascular conditions (e.g., elevated cholesterol and triglyceride levels). It is contemplated that the compounds of Formula (I) of the present invention, or pharmaceutically acceptable salts, solvates, or esters thereof, can be useful in treating one or more the conditions or diseases listed above. In particular, the compounds of Formula (I) of the present invention are useful in treating obesity.

“Effective amount” or “therapeutically effective amount” is meant to describe an amount of compound or a composition of the present invention effective in antagonizing a CB₁ receptor and thus producing the desired therapeutic effect in a suitable patient.

The selective CB₁ receptor antagonist compound of Formula (I), or a pharmaceutically acceptable salt, solvate, or ester thereof, can be administered in a therapeutically effective amount and manner to treat the specified condition. The daily dose of the selective CB₁ receptor antagonist of Formula (I) (or pharmaceutically acceptable salts, solvates, or esters thereof) administered to a mammalian patient or subject can range from about 1 mg/kg to about 50 mg/kg (where the units mg/kg refer to the amount of selective CB₁ receptor antagonist compound of Formula (I) per kg body weight of the patient), or about 1 mg/kg to about 25 mg/kg, or about 1 mg/kg to about 10 mg/kg.

Alternatively, the daily dose can range from about 1 mg to about 50 mg, or about 1 mg to about 25 mg, or about 5 mg to about 20 mg. Although a single administration of the selective CB₁ receptor antagonist compound of Formula (I), or salts, solvates, or esters thereof, can be efficacious, multiple dosages can also be administered. The exact dose, however, can readily be determined by the attending clinician and will depend on such factors as the potency of the compound administered, the age, weight, condition and response of the patient.

The treatment compositions of the present invention can be administered in any conventional dosage form, preferably an oral dosage form such as a capsule, tablet, powder, cachet, suspension or solution. The formulations and pharmaceutical compositions can be prepared using conventional pharmaceutically acceptable and conventional techniques.

In the veterinary context, in particular, the compounds of this invention can be administered to an animal patient in one or more of a variety of routes. For example, the compounds may be administered orally via, for example, a capsule, bolus, tablet (e.g., a chewable treat), powder, drench, elixir, cachet, solution, paste, suspension, or drink (e.g., in the drinking water or as a buccal or sublingual formulation). The compounds may alternatively (or additionally) be administered via a medicated feed (e.g., when administered to a non-human animal) by, for example, being dispersed in the feed or used as a top dressing or in the form of pellets or liquid which is added to the finished feed or fed separately. The compounds also may be administered (alternatively or additionally) parenterally via, for example, an implant or an intramural, intramuscular, intravascular, intratracheal, or subcutaneous injection. It is contemplated that other administration routes (e.g., topical, intranasal, rectal, etc.) may be used as well. Formulations for any such administration routes can be prepared using, for example, various conventional techniques known in the art. In some embodiments, from about 5 to about 70% by weight of the veterinary formulation (e.g., a powder or tablet) comprises active ingredient.

Suitable solid carriers are known in the art, and include, for example, magnesium carbonate, magnesium stearate, talc, sugar, and lactose. Tablets, powders, cachets, and capsules can be used as solid dosage forms suitable for oral administration.

To prepare suppositories, the active ingredient may be dispersed homogeneously into a melted wax that melts at low temperatures (e.g., a mixture of fatty acid glycerides or cocoa butter). Such dispersion may be achieved by, for example, stirring. The molten homogeneous mixture may be poured into convenient-sized molds, allowed to cool, and, thereby, solidify.

Liquid form preparations include solutions, suspensions, and emulsions. In some embodiments, for example, water or water-propylene glycol solutions are used for parenteral injection. Liquid form preparations also may include solutions for intranasal administration.

Aerosol preparations suitable for inhalation may include solutions and solids in powder form, which may be combined with a pharmaceutically acceptable carrier, such as an inert compressed gas.

Solid form preparations also include, for example, preparations that are intended to be converted, shortly before use, to liquid form preparations for either oral or parenteral administration. Such liquid forms include solutions, suspensions, and emulsions.

In some embodiments, the compounds of this invention are formulated for transdermal delivery. Transdermal compositions may be, for example, creams, lotions, aerosols, and/or emulsions, and can be included in a transdermal patch of the matrix or reservoir type as are conventional in the art for this purpose.

It is contemplated that the active can be incorporated into animal feed. A suitable amount of compound of the present invention can be placed into a commercially available feed product to achieve desired dosing levels. The amount of compound of the present invention incorporated into the feed will depend on the rate at which the animals are fed. Compounds or compositions of the present invention can be incorporated into feed mixtures before pelleting. Alternatively, the medicated feed is formed by coating feed pellets with a compound(s) or compositions of the present invention.

In some embodiments, the present invention provides a method of treating fish for an indication described herein. Such methods include administering an effective amount of an inventive compound (or compounds) of the invention (optionally together with one or more additional active agents as described herein) to a fish or a fish population. Administration generally is achieved by either feeding the fish an effective amount of the inventive compound or by immersing the fish in a solution that contains an effective amount of the inventive compound. It is to be further understood that the inventive compound can be administered by application of the inventive compound(s) to a pool or other water-holding area containing the animal, and allowing the fish to absorb the compound through its gills, or otherwise allowing the dosage of the inventive compound to be taken in. For individual treatment of specific animals, such as a particular fish (e.g., in a veterinary or aquarium setting), direct injection or injection of osmotic release devices comprising the inventive compound, alone or in combination with other agents, is an optional method of administering the inventive compound. Suitable routes of administration include, for example, intravenous, subcutaneous, intramuscular, spraying, dipping, or adding the compound directly into the water in a holding volume.

In other embodiments, the present invention provides a composition comprising: (a) at least one compound of Formula (I), or a pharmaceutically acceptable salt, solvate, isomer or ester thereof, and (b) at least one additional active ingredient. Thus, it is contemplated that any of the indications suitable for treatment by at least one compound of Formula (I) may be treated using at least one compound of Formula (I) together with at least one additional active ingredient. Such additional active ingredient(s) may be combined with one or more compounds of the invention to form a single composition for use or the active ingredients may be formulated for separate (simultaneous or sequential) administration. Such additional active ingredients are described herein or are know to those of ordinary skill in the art. Non-limiting examples include centrally acting agents and peripherally acting agents. Non-limiting examples of centrally acting agents include histamine-3 receptor antagonists such as those disclosed in U.S. Pat. No. 6,720,328 (incorporated herein by reference). Non-limiting examples of such histamine H-3 receptor antagonists include the compound having a structure (as well as salts, solvates, isomers, esters, prodrugs, etc. thereof):

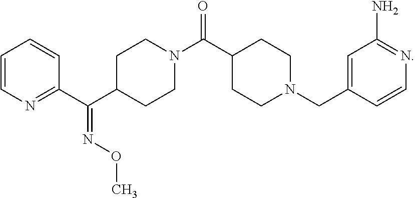 [SMILES: CO/N=C(/c1ccccn1)C1CCN(C(=O)C2CCN(Cc3ccnc(N)c3)CC2)CC1]

Other non-limiting examples of histamine-3 receptor antagonists include those disclosed in U.S. Pat. No. 7,105,505 (incorporated herein by reference). Non-limiting examples of such histamine H-3 receptor antagonists include the compound having a structure (as well as salts, solvates, isomers, esters, prodrugs, etc. thereof):

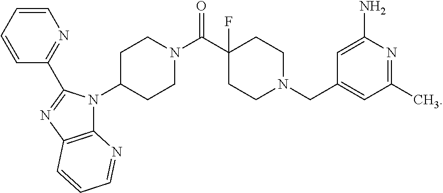 [SMILES: Cc1cc(CN2CCC(F)(C(=O)N3CCC(n4c(-c5ccccn5)nc5cccnc54)CC3)CC2)cc(N)n1]

Additional non-limiting examples of centrally acting agents include neuropeptide Y5 (NPY5) antagonists such as those disclosed in U.S. Pat. No. 6,982,267 (incorporated herein by reference). Non-limiting examples of such histamine NPY5 receptor antagonists include the compound having a structure (and salts, solvates, isomers, esters, prodrugs, etc. thereof):

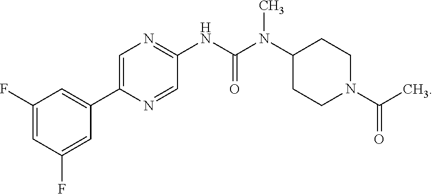 [SMILES: CC(=O)N1CCC(N(C)C(=O)Nc2cnc(-c3cc(F)cc(F)c3)cn2)CC1]

Non-limiting examples of peripherally acting agents include microsomal triglyceride transfer protein (MTP) inhibitors. Non-limiting examples of MTP inhibitors include dirlotapide (Slentrol™, Pfizer). Additional non-limiting examples of additional active ingredients are described herein.

In another embodiment, the present invention provides a composition comprising: (a) at least one compound of Formula (I), or a pharmaceutically acceptable salt, solvate, isomer or ester thereof, and (b) at least one cholesterol lowering compound.

Therapeutic combinations also are provided comprising: (a) a first amount of at least one selective CB₁ receptor antagonist, or a pharmaceutically acceptable salt, solvate, isomer or ester thereof; and (b) a second amount of at least one cholesterol lowering compound, wherein the first amount and the second amount together comprise a therapeutically effective amount for the treatment or prevention of a vascular condition, diabetes, obesity, hyperlipidemia, metabolic syndrome, or lowering a concentration of a sterol in the plasma of a subject.

Pharmaceutical compositions for the treatment or prevention of a vascular condition, diabetes, obesity, hyperlipidemia, metabolic syndrome, or lowering a concentration of a sterol in the plasma of a subject comprising a therapeutically effective amount of the above compositions or therapeutic combinations and a pharmaceutically acceptable carrier also are provided.

In still yet another embodiment, the compositions and combinations of the present invention comprise at least one compound of Formula (I), or a pharmaceutically acceptable salt, solvate, isomer, or ester thereof, and one or more anti-diabetic drugs. Non-limiting examples of anti-diabetic drugs include sulffonyl ureas, meglitinides, biguanides, thiazolidinediones, alpha glucosidase inhibitors, incretin mietics, DPP-IV (dipeptidyl peptidase-4 or DPP-4) inhibitors, amylin analogues, insulin (including insulin by mouth), and herbal extracts.

Non-limiting examples of sulfonylureas include tolbutamide (Orinase®), acetohexamide (Dymelor®), tolazamide (Tolinase®), chlorpropamide (Diabinese®), glipizide (Glucotrol(RO), glyburide (Diabeta®, Micronase®, and Glynase®), glimepiride (Amaryl®), and gliclazide (Diamicron®).

Non-limiting examples of meglitinides include repaglinide (Prandin®), and mateglinide (Starlix®).

Non-limiting examples of biguanides include metformin (Glucophage®).

Non-limiting examples of thaizolidinediones, also known as glitazines, include rosiglitazone (Avandia®), pioglitazone (Actos®), and troglitazine (Rezulin®).

Non-limiting examples of gludosidase inhibitors include miglitol (Glyset®) and acarbose (Precose/Glucobay®).

Non-limiting examples of incretin mimetics include GLP agonists such as exenatide and exendin-4, marketed as Byetta® (Amylin Pharmaceuticals, Inc. and Eli Lilly and Company.)

Non-limiting examples of Amylin analogues include pramlintide acetate (Symlin® Amylin Pharmaceuticals, Inc.).

Non-limiting examples of DPP4 inhibitors and other anti-diabetic drugs include the following: sitagliptin (marketed as Januvia®, available from Merck, pyrazine-based DPP-IV derivatives such as those disclosed in WO-2004085661, bicyclictetrahydropyrazine DPP IV inhibitors such as those disclosed in WO-03004498, PHX1149 (available from Phenomix, Inc.), ABT-279 and ABT-341 (available from Abbott, see WO-2005023762 and WO-2004026822), ALS-2-0426 (available Alantos and Servier), AR12243 (available from Arisaph Pharmaceuticals Inc., U.S. Pat. No. 6,803,357 and U.S. Pat. No. 6,890,898), boronic acid DPP-IV inhibitors such as those described in U.S. patent application Ser. No. 06/303,661, BI-A and BI-B (available from Boehringer Ingelheim), xanthine-based DPP-IV inhibitors such as those described in WO-2004046148, WO-2004041820, WO-2004018469, WO-2004018468 and WO-2004018467, saxagliptin (Bristol-Meyers Squibb and Astra Zenica), Biovitrim (developed by Santhera Pharmaceuticals (formerly Graffinity)), MP-513 (Mitsubishi Pharma), NVP-DPP-728 (qv) and structurally related 1-((S)-gamma-substituted prolyl)-(S)-2-cyanopyrrolidine compounds and analogs of NVP-DPP-728 (qv), DP-893 (Pfizer), vildagliptin (Novartis Institutes for BioMedical Research Inc), tetrahydroisoquinoline 3-carboxamide derivatives such as those disclosed in U.S. patent application Ser. No. 06/172081, N-substituted 2-cyanopyrrolidines, including LAF-237, such as those disclosed in PCT Publication Nos. WO-00034241, WO-00152825, WO-02072146 and WO-03080070, WO-09920614, WO-00152825 and WO-02072146, SYR-322 (Takeda), denagliptin, SNT-189546, Ro-0730699, BMS-2, Aurigene, ABT-341, Dong-A, GSK-2, HanAll, LC-15-0044, SYR-619, Bexel, alogliptin benzoate, and ALS-2-0426. Non-limiting examples of other anti-diabetic drugs include metformin, thiazolidinediones (TZD), and sodium glucose cotransporter-2 inhibitors such as dapagliflozin (Bristol Meyers Squibb) and sergliflozin (GlaxoSmithKline), and FBPase (fructose 1,6-bisphosphatase) inhibitors.

In still yet another embodiment, the compositions and combinations of the present invention comprise at least one compound of Formula (I), or a pharmaceutically acceptable salt, solvate, isomer or ester thereof, and at least one sterol absorption inhibitor or at least one 5α-stanol absorption inhibitor.

In still yet another embodiment of the present invention, there is provided a therapeutic combination comprising: (a) a first amount of at least one compound of Formula (I), or a pharmaceutically acceptable salt, solvate, isomer or ester thereof; and (b) a second amount of at least one cholesterol lowering compound; wherein the first amount and the second amount together comprise a therapeutically effective amount for the treatment or prevention of one or more of a vascular condition, diabetes, obesity, metabolic syndrome, or lowering a concentration of a sterol in the plasma of a subject.

In still yet another embodiment, the present invention provides for a pharmaceutical composition for the treatment or prevention of one or more of a vascular condition, diabetes, obesity, metabolic syndrome, or lowering a concentration of a sterol in the plasma of a subject, comprising a therapeutically effective amount of a composition or therapeutic combination comprising: (a) at least one compound of Formula (I), or a pharmaceutically acceptable salt, solvate, or isomer ester thereof; (b) a cholesterol lowering compound; and (c) a pharmaceutically acceptable carrier.

As used herein, “therapeutic combination” or “combination therapy” means the administration of two or more therapeutic agents, such as a compound according to Formula (I) of the present invention, and a cholesterol lowering compound such as one or more substituted azetidinone or one or more substituted β-lactam, to prevent or treat a condition, for example a vascular condition, such as hyperlipidaemia (for example atherosclerosis, hypercholesterolemia or sitosterolemia), vascular inflammation, metabolic syndrome, stroke, diabetes, obesity and/or reduce the level of sterol(s) (such as cholesterol) in the plasma or tissue. As used herein, “vascular” comprises cardiovascular, cerebrovascular and combinations thereof. The compositions, combinations and treatments of the present invention can be administered by any suitable means which produce contact of these compounds with the site of action in the body, for example in the plasma, liver, small intestine, or brain (e.g., hippocampus, cortex, cerebellum, and basal ganglia) of a subject (mammal or human or other animal). Such administration includes co-administration of these therapeutic agents in a substantially simultaneous manner, such as in a single tablet or capsule having a fixed ratio of active ingredients or in multiple, separate capsules for each therapeutic agent. Also, such administration includes the administration of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

As discussed above, the compositions, pharmaceutical compositions and therapeutic combinations of the present invention comprise: (a) one or more compounds according to Formula (I) of the present invention, or pharmaceutically acceptable salts, solvates, isomers or esters thereof; and (b) one or more cholesterol lowering agents. A non-limiting list of cholesterol lowering agents useful in the present invention include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin (for example LESCOL®), cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), Pravastatin (marketed as LIVALO®), cerivastatin, itavastatin (or pitavastatin, NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol (e.g., cholesterol) biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methylpyrazine-2-carboxylic acid 4-oxide), and niacin extended-release tablets such as NIASPAN®; clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChoi® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA:Cholesterol O-acyltransferase (“ACAT”) Inhibitors such as avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N—[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; nicotinic acid receptor agonists (e.g., agonists of the HM74 and HM74A receptor which receptor is described in US 2004/0142377, US 2005/0004178, US 2005/0154029, U.S. Pat. No. 6,902,902, WO 2004/071378, WO 2004/071394, WO 01/77320, US 2003/0139343, WO 01/94385, WO 2004/083388, US 2004/254224, US 2004/0254224, US 2003/0109673 and WO 98/56820) for example those described in WO 2004/033431, WO 2005/011677, WO 2005/051937, US 2005/0187280, US 2005/0187263, WO 2005/077950, WO 2005/016867, WO 2005/016870, WO2005061495, WO2006005195, WO2007059203, US2007105961, CA2574987, and AU2007200621; and the substituted azetidinone or substituted β-lactam sterol absorption inhibitors discussed in detail below.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a patient (e.g., mammal or human). Non-limiting examples of stanol absorption inhibitors include those compounds that inhibit cholesterol absorption in the small intestine. Such compounds are well known in the art and are described, for example, in U.S. RE 37,721; U.S. Pat. No. 5,631,356; U.S. Pat. No. 5,767,115; U.S. Pat. No. 5,846,966; U.S. Pat. No. 5,698,548; U.S. Pat. No. 5,633,246; U.S. Pat. No. 5,656,624; U.S. Pat. No. 5,624,920; U.S. Pat. No. 5,688,787; U.S. Pat. No. 5,756,470; US Publication No. 2002/0137689; WO 02/066464; WO 95/08522 and WO96/19450. Non-limiting examples of cholesterol absorption inhibitors also include non-small molecule agents, microorganisms such as Bifidobacterium animalis subsp. animalis YIT 10394, Bifidobacterium animalis subsp. lactis JCM 1253, Bifidobacterium animalis subsp. lactis JCM 7117 and Bifidobacterium Pseudolongum subsp. Globosum, which are described, e.g., in WO2007029773. Each of the aforementioned publications is incorporated by reference.

### Substituted Azetidinones of Formula (II)

In one embodiment, substituted azetidinones useful in the compositions, therapeutic combinations and methods of the present invention are represented by Formula (II) below:

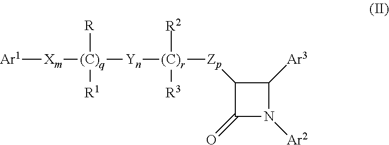

or pharmaceutically acceptable salts, solvates, or esters of the compounds of Formula (II), wherein, in Formula (II) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(lower alkyl)₂-;

R and R² are independently selected from the group consisting of —OR⁶, —OC(O)R⁶, —OC(O)OR⁹ and —OC(O)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀CONR⁶R⁷, -(lower alkylene)COOR⁶, —CH═CH—C(O)OR⁶, —CF₃, —CN, —NO₂ and halogen;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —SO₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Certain compounds useful in the therapeutic compositions or combinations of the invention may have at least one asymmetrical carbon atom and therefore all isomers, including enantiomers, diastereomers, stereoisomers, rotamers, tautomers and racemates of the compounds of Formula II-XIII (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formulae II-XIII. Isomers may also include geometric isomers, e.g., when a double bond is present.

Those skilled in the art will appreciate that for some of the compounds of the Formulae II-XIII, one isomer may show greater pharmacological activity than other isomers.

Preferred compounds of Formula (II) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴— and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —OC(O)R⁶, —OC(O)OR⁹ and —OC(O)NR⁶R⁷, defined above).

The sum of m, n, p, q and r is preferably 2, 3 or 4, more preferably 3. Preferred are compounds OF Formula (II) wherein m, n and r are each zero, q is 1 and p is 2.

Also preferred are compounds of Formula (II) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (II) wherein p, q and n are each zero, r is l, m is 2, X is —CH₂— and R² is —OR⁶, especially when R⁶ is hydrogen.

Another group of preferred compounds of Formula (II) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

### Substituted Azetidinones of Formula (III)

In a preferred embodiment, a substituted azetidinone of Formula (II) useful in the compositions, therapeutic combinations and methods of the present invention is represented by Formula (III) (ezetimibe) below:

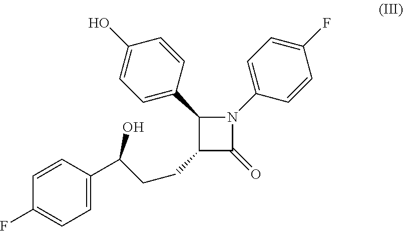 [SMILES: O=C1[C@H](CC[C@H](O)c2ccc(F)cc2)[C@@H](c2ccc(O)cc2)N1c1ccc(F)cc1]

or pharmaceutically acceptable salts, solvates, or esters of the compound of Formula (III). The compound of Formula (III) can be in anhydrous or hydrated form. A product containing ezetimibe compound is commercially available as ZETIA® ezetimibe formulation from MSP Pharmaceuticals.

Compounds of Formula (II) can be prepared by a variety of methods well known to those skilled in the art, for example such as are disclosed in U.S. Pat. Nos. 5,631,365, 5,767,115, 5,846,966, 6,207,822, 6,627,757, 6,093,812, 5,306,817, 5,561,227, 5,688,785, and 5,688,787, each of which is incorporated herein by reference.

### Substituted Azetidinones of Formula (IV)

Alternative substituted azetidinones useful in the compositions, therapeutic combinations and methods of the present invention are represented by formula (IV) below:

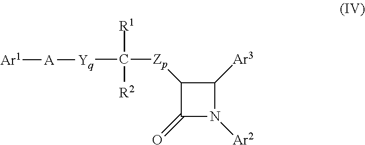

or a pharmaceutically acceptable salt thereof or a solvate thereof, or an ester thereof, wherein, in Formula (IV) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(lower alkyl)₂-;

A is selected from —O—, —S—, —S(O)— or —S(O)₂—;

R¹ is selected from the group consisting of —OR⁶, —OC(O)R⁶, —OC(O)OR⁹ and —OC(O)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —OC(O)R⁶, —OC(O)OR⁹, —O(CH₂)₁₋₅OR⁹, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂-lower alkyl, —NR⁶S(O)₂-aryl, —C(O)NR⁶R⁷, —COR^{E}, —SO₂NR⁶R⁷, S(O)₀₋₂-alkyl, S(O)₀₋₂-aryl, —O(CH₂)₁₋₁₀—C(O)OR⁶, —O(CH₂)₁₋₁₀C(O)NR⁶R⁷, o-halogeno, m-halogeno, o-lower alkyl, m-lower alkyl, -(lower alkylene)-C(O)OR⁶, and —CH═CH—C(O)OR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Methods for making compounds of Formula (IV) are well known to those skilled in the art. Non-limiting examples of suitable methods are disclosed in U.S. Pat. No. 5,688,990, which is incorporated herein by reference.

### Substituted Azetidinones of Formula (V)

In another embodiment, substituted azetidinones useful in the compositions, therapeutic combinations and methods of the present invention are represented by Formula (V):

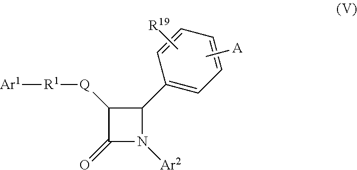

or a pharmaceutically acceptable salt thereof or a solvate thereof, or an ester thereof, wherein, in Formula (V) above:

A is selected from the group consisting of R²-substituted heterocycloalkyl, R²-substituted heteroaryl, R²-substituted benzo-fused heterocycloalkyl, and R²-substituted benzo-fused heteroaryl;

Ar¹ is aryl or R³-substituted aryl;

Ar² is aryl or R⁴-substituted aryl;

Q is a bond or, with the 3-position ring carbon of the azetidinone, forms the spiro group

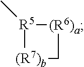

and

R¹ is selected from the group consisting of:
—(CH₂)_{q}—, wherein q is 2-6, provided that when Q forms a spiro Ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

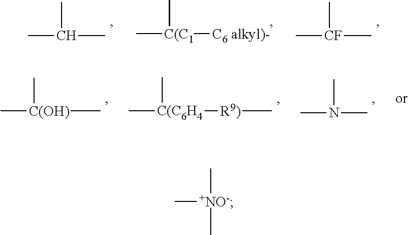

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶ is can be the same or different; and provided that when b is 2 or 3, the R⁷'s can be the same or different;

and when Q is a bond, R¹ also can be selected from:

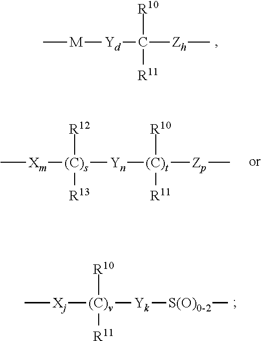

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —OC(O)R¹⁴, —OC(O)OR¹⁶ and —OC(O)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁶C(O)(C₁-C₆ alkylene)-, —NHC(O)R¹⁶, OH, C₁-C₆ alkoxy, —OC(O)R¹⁶, —C(O)R¹⁴, hydroxy(C₁-C₆)alkyl, (C₁-C₆)alkoxy(C₁-C₆)alkyl, NO₂, —S(O)₀₋₂R¹⁶, —S(O)₂NR¹⁴R¹⁵ and —(C₁-C₆ alkylene)C(O)OR¹⁴; when R² is a substituent on a heterocycloalkyl ring, R² is as defined, or R² is ═O or

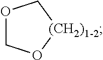

and, where R² is a substituent on a substitutable ring nitrogen, R² is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

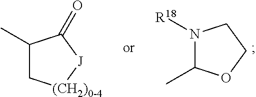

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —OC(O)R¹⁴, —OC(O)OR¹⁶, —O(CH₂)₁₋₅OR¹⁴, —OC(O)NR¹⁴R¹⁵, —NR¹⁴R¹⁵, —NR¹⁴C(O)R¹⁵, —NR¹⁴C(O)OR¹⁶, —NR¹⁴C(O)NR¹⁵R¹⁹, —NR¹⁴S(O)₂R¹⁶, —C(O)OR¹⁴, —C(O)NR¹⁴R¹⁵, —C(O)R¹⁴, —S(O)₂NR¹⁴R¹⁵, S(O)₀₋₂R¹⁶, —O(CH₂)₁₋₁₀—C(O)OR¹⁴, —O(CH₂)₁₋₁₀C(O)NR¹⁴R¹⁵, —(C₁-C₆ alkylene)-C(O)OR¹⁴, —CH═CH—C(O)OR¹⁴, —CF₃, —CN, —NO₂ and halogen;

R⁸ is hydrogen, (C₁-C₆)alkyl, aryl (C₁-C₆)alkyl, —C(O)R¹⁴ or —C(O)OR¹⁴;

R⁹ and R¹⁷ are independently 1-3 groups independently selected from the group consisting of hydrogen, (C₁-C₆)alkyl, (C₁-C₆)alkoxy, —C(O)OH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy. Methods for making compounds of Formula (V) are well known to those skilled in the art. Non-limiting examples of suitable methods are disclosed in U.S. Pat. No. 5,656,624, which is incorporated herein by reference.

### Substituted Azetidinones of Formula (VI)

In another embodiment, substituted azetidinones useful in the compositions, therapeutic combinations and methods of the present invention are represented by Formula (VI):

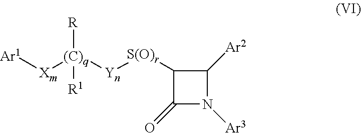

or a pharmaceutically acceptable salt thereof or a solvate thereof, or an ester thereof, wherein, in Formula (VI) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(lower alkyl)₂-;

R is —OR⁶, —OC(O)R⁶, —OC(O)OR⁹ or —OC(O)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, —CF₃, —CN, —NO₂, halogen, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, —S(O)₀₋₂R⁹, —O(CH₂)₁₋₁₀—C(O)OR⁶, —O(CH₂)₁₋₁₀C(O)NR⁶R⁷, —CF₃, —CN, —NO₂ and halogen.

Methods for making compounds of Formula (VI) are well known to those skilled in the art. Non-limiting examples of suitable methods are disclosed in U.S. Pat. No. 5,624,920, which is incorporated herein by reference.

### Substituted Azetidinones of Formula (VII)

In another embodiment, substituted azetidinones useful in the compositions, therapeutic combinations and methods of the present invention are represented by Formula (VII):

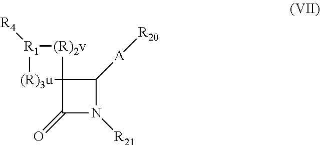

or a pharmaceutically acceptable salt thereof or a solvate thereof, or an ester thereof, wherein:

R¹ is:

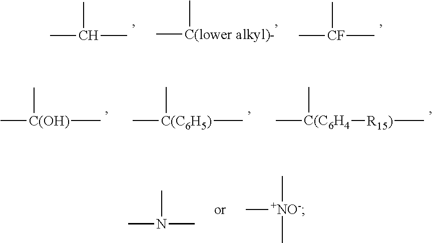

R² and R³ are independently selected from the group consisting of: —CH₂—, —CH(lower alkyl)-, —C(lower alkyl)₂-, —CH═CH— and —C(lower alkyl)=CH—; or R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, each R² can be the same or different; and provided that when u is 2 or 3, each R³ can be the same or different;

R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ—Z—(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-;
B—(C₄-C₆ alkadienylene)-; B—(CH₂)ₜ—Z—(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;
   B—(CH₂)ₐ—Z—(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is a C₃-C₆ cycloalkyl and s is 0, 1, 2, 3, 4, 5 or 6; or

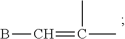

R¹ and R⁴ together form the group

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

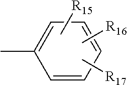

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹(O)₂SNH—, (R¹¹(O)₂S)₂N—, —S(O)₂NH₂, —S(O)₀₋₂R⁸, tert-butyldimethyl-silyloxymethyl, —C(O)R¹², —C(O)OR¹⁹, —C(O)N(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and

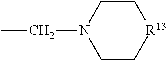

for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —C(O)OH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy,

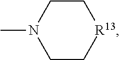

—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzo-fused heteroaryl, W-substituted benzo-fused heteroaryl and cyclopropyl, wherein heteroaryl is as defined above.

Methods for making compounds of Formula (VII) are well known to those skilled in the art. Non-limiting examples of suitable methods are disclosed in U.S. Pat. No. 5,698,548, which is incorporated herein by reference.

### Substituted Azetidinones of Formula (VIII)

In another embodiment, substituted azetidinones useful in the compositions, therapeutic combinations and methods of the present invention are represented by Formulas (VIIIA) and (VIIIB):

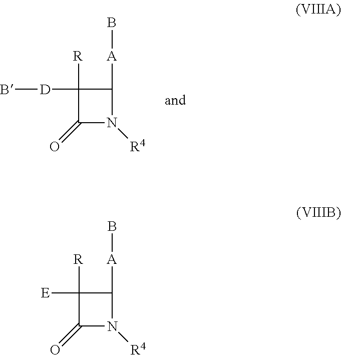

or a pharmaceutically acceptable salt, solvate, or ester thereof, wherein:

A is —CH═CH—, —C≡C— or —(CH₂)ₚ— wherein p is 0, 1 or 2;

B is

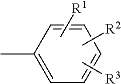

B′ is

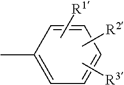

D is —(CH₂)ₘC(O)— or —(CH₂)_{q}— wherein m is 1, 2, 3 or 4 and q is 2, 3 or 4;

E is C₁₀ to O₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R¹′, R²′, and R³′ are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶(O)₂SNH— and —S(O)₂NH₂;

R⁴ is

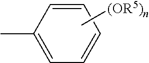

wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt, solvate, or ester thereof.

### Sterol Absorption Inhibitors of Formula (IX)

In another embodiment, sterol absorption inhibitors useful in the compositions and methods of the present invention are represented by Formula (IX):

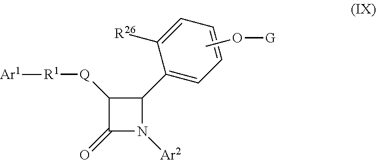

or a pharmaceutically acceptable salt, solvate, or ester thereof, wherein, in Formula (IX) above,

R²⁶ is H or OG¹;

G and G¹ are independently selected from the group consisting of H,

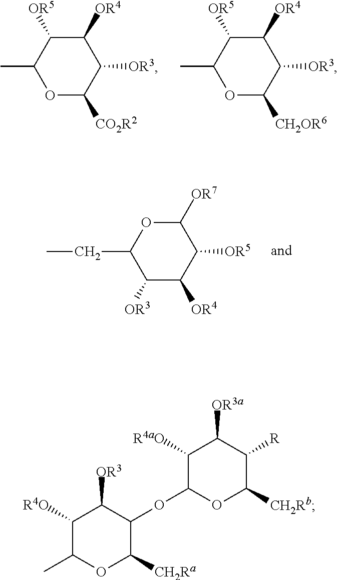

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and

R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O) —N((C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

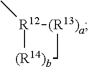

and

R¹ is selected from the group consisting of
—(CH₂)_{q}—, wherein q is 2-6, provided that when Q forms a spiro Ring,

q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6; —(C₂-C₆)alkenylene-; and
—(CH₂)_{f}—V—(CH₂)_{g}—, wherein V is C₃-C₆ cycloalkylene, f is 1-5 and g is 0-5, provided that the sum off and g is 1-6;

R¹² is:

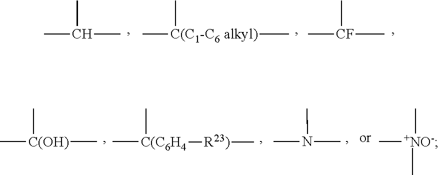

R¹³ and R¹⁴ are independently selected from the group consisting of

—CH₂—, —CH((C₁-C₆) alkyl)-, —C((C₁-C₆) alkyl)₂, —CH═CH— and —C((C₁-C₆) alkyl)=CH—; or

R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, each R¹³ can be the same or different; and

provided that when b is 2 or 3, each R¹⁴ can be the same or different; and when Q is a bond, R¹ also can be:

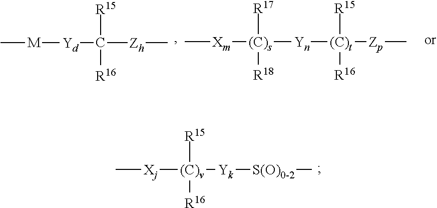

M is —O—, —S—, —S(O)— or —S(O)₂—; X, Y and Z are independently selected from the group consisting of —CH₂—, —CH(C₁-C₆)alkyl- and —C((C₁-C₆)alkyl)₂;

R¹⁰ and R¹¹ are independently selected from the group consisting of 1-3 substituents independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR₁₉C(O)R²⁰, —NR¹⁹C(O)OR²¹, —NR¹⁹C(O)NR²OR²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halogen;

R¹⁵ and R¹⁷ are independently selected from the group consisting of —OR¹⁹, —OC(O)R¹⁹, —OC(O)OR²¹ and —OC(O)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and

provided that when p is 0 and s is 1, the sum of m, t and n is 1-5;

v is 0 or 1;

j and k are independently 1-5, provided that the sum of j, k and v is 1-5;

and when Q is a bond and R¹ is

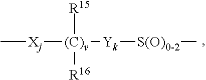

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are independently 1-3 groups independently selected from the group consisting of H, (C₁-C₆)alkyl, (C₁-C₆)alkoxy, —C(O)OH, NO₂, —NR¹⁹R²⁰, —OH and halogeno; and

R²⁵ is H, —OH or (C₁-C₆)alkoxy.

Methods for making compounds of Formula (IX) are well known to those skilled in the art. Non-limiting examples of suitable methods are disclosed in U.S. Pat. No. 5,756,470, which is incorporated herein by reference.

### Substituted Azetidinones of Formula (X)

In another embodiment, substituted azetidinones useful in the compositions and methods of the present invention are represented by Formula (X) below:

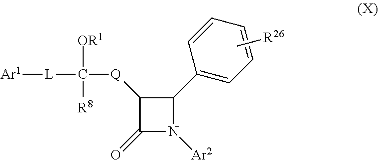

or a pharmaceutically acceptable salt, solvate, or ester thereof, wherein in Formula (X):

R¹ is selected from the group consisting of H, G, G¹, G², —SO₃H and —PO₃H;

G is selected from the group consisting of: H,

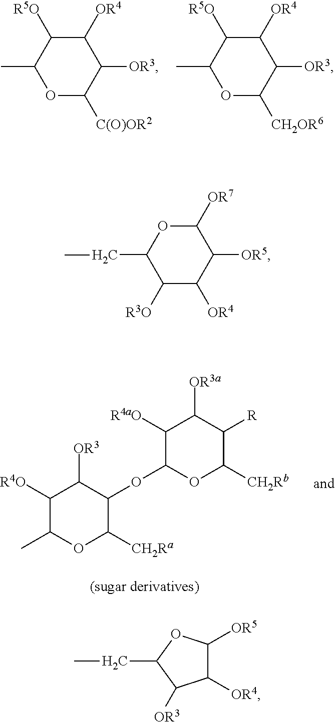

wherein R, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N(C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

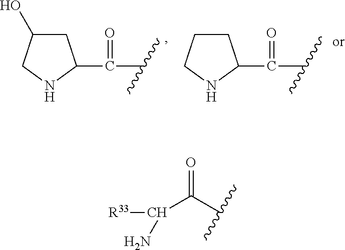

G¹ is represented by the structure:

wherein R³³ is independently selected from the group consisting of unsubstituted alkyl, R³⁴-substituted alkyl, (R³⁵)(R³⁶)alkyl-,

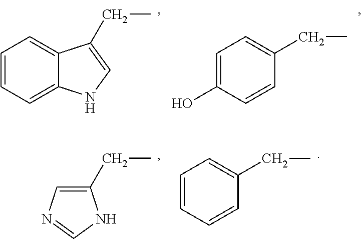

R³⁴ is one to three substituents, each R³⁴ being independently selected from the group consisting of HO(O)C—, HO—, HS—, (CH₃)S—, H₂N—, (NH₂)(NH)C(NH)—, (NH₂)C(O)— and HO(O)CCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure:

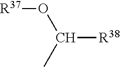

wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:
a) a covalent bond;
b) —(CH₂)_{q}—, wherein q is 1-6;
c) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
d) —(C₂-C₆)alkenylene-;
e) —(CH₂)_{f}—V—(CH₂)_{g}—, wherein V is C₃-C₆cycloalkylene, f is 1-5 and g is 0-5, provided that the sum of f and g is 1-6; and
f)

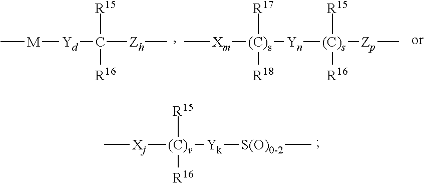

wherein M is —O—, —S—, —S(O)— or —S(O)₂—;

X, Y and Z are each independently selected from the group consisting of —CH₂—, —CH(C₁-C₆)alkyl- and —C((C₁-C₆)alkyl)₂-;

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR¹⁹C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl; or

R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group

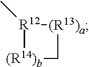

wherein R¹² is

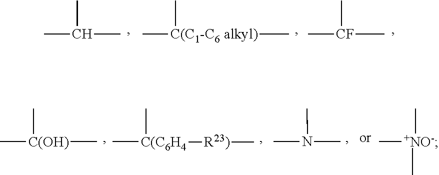

R¹³ and R¹⁴ are each independently selected from the group consisting of —CH₂—, —CH(C₁-C₆ alkyl)-, —C((C₁-C₆) alkyl)₂, —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, each R¹³ can be the same or different; and provided that when b is 2 or 3, each R¹⁴ can be the same or different;

and when Q is a bond and L is

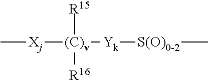

then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are each independently selected from the group consisting of 1-3 substituents which are each independently selected from the group consisting of H, (C₁-C₆)alkyl, (C₁-C₆)alkoxy, —C(O)OH, NO₂, —NR¹⁹R²⁰, —OH and halo; and

R²⁵ is H, —OH or (C₁-C₆)alkoxy.

Examples of compounds of Formula (X) which are useful in the methods and combinations of the present invention and methods for making such compounds are disclosed in U.S. patent application Ser. No. 10/166,942, filed Jun. 11, 2002, incorporated herein by reference.

### Substituted Azetidinones of Formulae (XI)-(XIII)

An example of a useful substituted azetidinone is one represented by the Formula (XI):

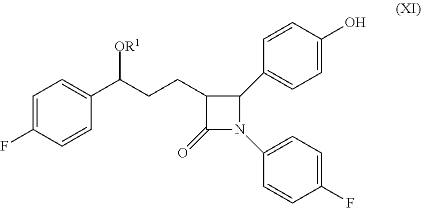

wherein R¹ is defined as above.

A more preferred compound is one represented by Formula (XII):

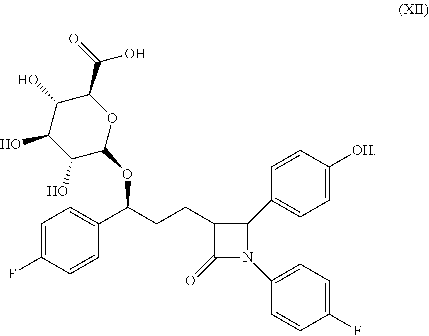

Another useful compound is represented by Formula (XIII):

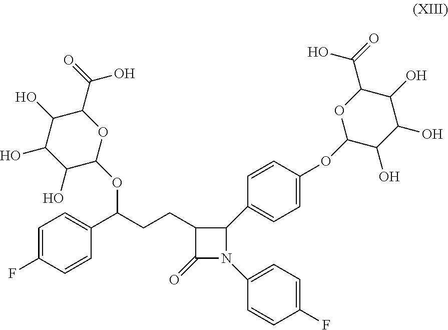 [SMILES: O=C(O)C1OC(Oc2ccc(C3C(CCC(OC4OC(C(=O)O)C(O)C(O)C4O)c4ccc(F)cc4)C(=O)N3c3ccc(F)cc3)cc2)C(O)C(O)C1O]

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, diphenyl azetidinones and derivatives disclosed in U.S. Patent Publication Nos. 2002/0039774, 2002/0128252, 2002/0128253 and 2002/0137689, 2004/063929, WO 2002/066464, U.S. Pat. Nos. 6,498,156 and 6,703,386, each of which is incorporated by reference herein.

Other sterol absorption inhibitors useful in the compositions, therapeutic combinations and methods of the present invention are described in WO 2004/005247, WO 2004/000803, WO 2004/000804, WO 2004/000805, WO 0250027, U.S. published application 2002/0137689, and the compounds described in L. Kvœrnø et al., Angew. Chem. Int. Ed., 2004, vol. 43, pp. 4653-4656, all of which are incorporated herein by reference. An illustrative compound of Kvœrnø et al. is:

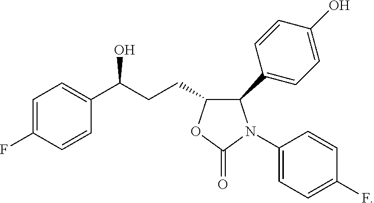 [SMILES: O=C1O[C@H](CC[C@H](O)c2ccc(F)cc2)[C@@H](c2ccc(O)cc2)N1c1ccc(F)cc1]

The compounds of Formulae II-XIII can be prepared by known methods, including the methods discussed above and, for example, in WO 93/02048, U.S. Pat. Nos. 5,306,817 and 5,561,227, herein incorporated by reference, which describe the preparation of compounds wherein —R¹-Q- is alkylene, alkenylene or alkylene interrupted by a hetero atom, phenylene or cycloalkylene; WO 94/17038 and U.S. Pat. No. 5,698,548, herein incorporated by reference, describe the preparation of compounds wherein Q is a spirocyclic group; WO 95/08532, U.S. Pat. No. 5,631,365, U.S. Pat. No. 5,767,115, U.S. Pat. No. 5,846,966, and U.S. R.E. 37,721, herein incorporated by reference, describe the preparation of compounds wherein —R¹-Q- is a hydroxy-substituted alkylene group; PCT/US95/03196, herein incorporated by reference, describes compounds wherein —R¹-Q- is a hydroxy-substituted alkylene attached to the Ar¹ moiety through an —O— or S(O)₀₋₂— group; and U.S. Ser. No. 08/463,619, filed Jun. 5, 1995, herein incorporated by reference, describes the preparation of compounds wherein —R¹-Q- is a hydroxy-substituted alkylene group attached to the azetidinone ring by a —S(O)₀₋₂— group. Each of the above patents or publications are herein incorporated by reference in their entirety.

The daily dose of the sterol absorption inhibitor(s) administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In another embodiment of the present invention, the compositions or therapeutic combinations described above comprise one or more selective CB₁ receptor antagonist compounds of Formula (I) in combination with one or more cholesterol biosynthesis inhibitors and/or lipid-lowering compounds discussed below.

Generally, a total daily dosage of cholesterol biosynthesis inhibitor(s) can range from about 0.1 to about 160 mg per day, and preferably about 0.2 to about 80 mg/day in single or 2-3 divided doses.

In another alternative embodiment, the compositions, therapeutic combinations or methods of the present invention can comprise at least one compound of Formula (I), or pharmaceutically acceptable salts, solvates, or esters thereof, and one or more bile acid sequestrants (insoluble anion exchange resins), co-administered with or in combination with the compound of Formula (I), or a pharmaceutically acceptable salt, solvate, or ester thereof, and a substituted azetidinone or a substituted β-lactam discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors that bind LDL from plasma to further reduce cholesterol levels in the blood.

Generally, a total daily dosage of bile acid sequestrant(s) can range from about 1 to about 50 grams per day, and preferably about 2 to about 16 grams per day in single or 2-4 divided doses.

In an alternative embodiment, the compositions or treatments of the present invention can comprise at least one compound of Formula (I), or pharmaceutically acceptable salts, solvates, or esters thereof, and one or more IBAT inhibitors. The IBAT inhibitors can inhibit bile acid transport to reduce LDL cholesterol levels. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions or treatments of the present invention can comprise at least one compound of Formula (I), or pharmaceutically acceptable salts, solvates, or esters thereof, and nicotinic acid (niacin) and/or derivatives thereof. Nicotinic acid and its derivatives inhibit hepatic production of VLDL and its metabolite LDL and increases HDL and apo A-1 levels. An example of a suitable nicotinic acid product is NIASPAN® (niacin extended-release tablets), which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or treatments of the present invention can comprise at least one compound of Formula (I), or pharmaceutically acceptable salts, solvates, or esters thereof, and one or more AcylCoA:Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Generally, a total daily dosage of ACAT inhibitor(s) can range from about 0.1 to about 1000 mg/day in single or 2-4 divided doses.

In another alternative embodiment, the compositions or treatments of the present invention can comprise at least one compound of Formula (I), or pharmaceutically acceptable salts, solvates, or esters thereof, and one or more Cholesteryl Ester Transfer Protein (“CETP”) Inhibitors, such as torcetrapib. CETP is responsible for the exchange or transfer of cholesteryl ester carrying HDL and triglycerides in VLDL. Pancreatic cholesteryl ester hydrolase (pCEH) inhibitors such as WAY-121898 also can be co-administered with or in combination.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or treatments of the present invention can comprise at least one compound of Formula (I), or pharmaceutically acceptable salts, solvates, or esters thereof, and probucol or derivatives thereof, which can reduce LDL levels.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the present invention can comprise at least one compound of Formula (I), or pharmaceutically acceptable salts, solvates, or esters thereof, and low-density lipoprotein (LDL) receptor activators.

Generally, a total daily dosage of LDL receptor activator(s) can range from about 1 to about 1000 mg/day in single or 2-4 divided doses.

In another alternative embodiment, the compositions or treatments of the present invention can comprise at least one compound of Formula (I), or pharmaceutically acceptable salts, solvates, or esters thereof, and fish oil. Generally, a total daily dosage of fish oil or Omega 3 fatty acids can range from about 1 to about 30 grams per day in single or 2-4 divided doses.

In another alternative embodiment, the compositions or treatments of the present invention can further comprise at least one compound of Formula (I), or pharmaceutically acceptable salts, solvates, or esters thereof, and natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions or treatments of the present invention can comprise at least one compound of Formula (I), or pharmaceutically acceptable salts, solvates, or esters thereof, and plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions or treatments of the present invention can comprise at least one compound of Formula (I), or pharmaceutically acceptable salts, solvates, or esters thereof, and antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions or treatments of the present invention can comprise at least one compound of Formula (I), or pharmaceutically acceptable salts, solvates, or esters thereof, and monocyte and macrophage inhibitors such as polyunsaturated fatty acids (PUFA), thyroid hormones including thyroxine analogues such as CGS-26214 (a thyroxine compound with a fluorinated ring), gene therapy and use of recombinant proteins such as recombinant apo E. Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17 α-dihydroequilin sulfate, sodium 17 α-estradiol sulfate, sodium 17 β-dihydroequilin sulfate, sodium 17 α-dihydroequilenin sulfate, sodium 17 β-dihydroequilenin sulfate, sodium equilenin sulfate and sodium 17 β-estradiol sulfate; available from Duramed Pharmaceuticals, Inc., Cincinnati, Ohio, under the tradename Cenestin;

(b) ethinyl estradiol (19-nor-17 α-pregna-1,3,5(10)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estropipate (piperazine estra-1,3,5(10)-trien-17-one, 3-(sulfooxy)-estrone sulfate); available from Pharmacia & Upjohn, Peapack, N.J., under the tradename Ogen and from Women First Health Care, Inc., San Diego, Calif., under the tradename Ortho-Est; and

(e) conjugated estrogens (17 α-dihydroequilin, 17 α-estradiol, and 17 β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1,3,5 (10)-triene-3,17β-diol hemihydrate) and norethindrone (17 β-acetoxy-19-nor-17 α-pregn-4-en-20-yn-3-one); which is available from Pharmacia & Upjohn, Peapack, N.J., under the tradename Activella;

(b) the combination of levonorgestrel (d(−)-13 β-ethyl-17 α-ethinyl-17 β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil;

(c) the combination of ethynodiol diacetate (19-nor-17 α-pregn-4-en-20-yne-3β,17-diol diacetate) and ethinyl estradiol; available from G.D. Searle & Co., Chicago, Ill., under the tradename Demulen and from Watson under the tradename Zovia;

(d) the combination of desogestrel (13-ethyl-1′-methylene-18,19-dinor-17α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and FemHRT, from Watson under the tradenames Microgestin, Necon, and Tri-Norinyl, from Ortho-McNeil under the tradenames Modicon and Ortho-Novum, and from Warner Chilcott Laboratories, Rockaway, N.J., under the tradename Ovcon;

(f) the combination of norgestrel ((±)-13-ethyl-17-hydroxy-18,19-dinor-17 α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17 α-pregna-1,3,5(10)-trien-20-yn-17-o1); available from Watson under the tradenames Brevicon and Norinyl;

(h) the combination of 17 β-estradiol (estra-1,3,5(10)-triene-3,17β-diol) and micronized norgestimate (17 α-17-(Acetyloxyl)-13-ethyl-18,19-dinorpregn-4-en-20-yn-3-one3-oxime); available from Ortho-McNeil under the tradename Ortho-Prefest;

(i) the combination of norgestimate (18,19-dinor-17-pregn-4-en-20-yn-3-one, 17-(acetyloxy)-13-ethyl-,oxime, (17(α)-(+)-) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20-dione, 17-(acetyloxy)-6-methyl-, (6(α))-pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

In another alternative embodiment, the compositions, therapeutic combinations or methods of the present invention can comprise at least one compound of Formula (I), or pharmaceutically acceptable salts, solvates, isomers or esters thereof, and one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors (such as milrinoone, theophylline, vinpocetine, EHNA (erythro-9-(2-hydroxy-3-monyl)adenine), sildenafil citrate, marketed as VIAGRA®, and tadalafil, marketed as Clalis®); compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can comprise at least one compound of Formula (I), or pharmaceutically acceptable salts, solvates, isomers or esters thereof, and one or more blood modifiers which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or lipid modulating agents discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (Abcoximab, aspirin, anagrelide hydrochloride, Beraprost, bivalirudin, cilostazol, Carbasalate calcium, Cloricromen, Clopidogrel, dalteparin sodium, danaparoid sodium, dazoxiben hydrochloride, Ditazole, Ditazole, Dipyridamole, Eptifibatide, efegatran sulfate, enoxaparin sodium, fluretofen, ifetroban, ifetroban sodium, Indobufen, Iloprost, lamifiban, lotrafiban hydrochloride, napsagatran, orbofiban acetate, Picotamide, Prasugrel, Prostacyclin, Treprostinil, Ticlopidine, Treprostinil, Triflusal, roxifiban acetate, sibrafiban, tinzaparin sodium, trifenagrel, abciximab, vitamin K antagonists, zolimomab aritox, enzymes such as Alteplase, Ancrod, Anistreplase, Brinase, Drotrecogin alfa, Fibrinolysin, Protein C, Reteplase, Saruplase, Steptokinase, Tenecteplase, and Urokinase), other antithrobotic agents such as Aragatroban, Bivalirudin, Dabigatran, Desirudin, Jirduin, Lepirudin, Melagatran, and Ximelagatran); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate (marketed as Plavix®), epoprostenol, epoprostenol sodium, ticlopidine hydrochloride, aspirin, ibuprofen, naproxen, sulindac, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor Vila inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl} amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl] propylamides, substituted n-[(aminomethyl)phenyl] propylamides, tissue factor pathway inhibitor (TFPI), low molecular weight heparins (such as dalteparin sodium, marketed as FRAGMIN®), heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can comprise at least one compound of Formula (I), or pharmaceutically acceptable salts, solvates, isomers or esters thereof, and one or more cardiovascular agents which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or PPAR receptor activators discussed above. Useful cardiovascular agents include but are not limited to calcium channel blockers (clentiazem maleate, amlodipine besylate (marketed as NORVASC® and LOTREL®), isradipine, nimodipine, felodipine (marketed as PLENDIL®), nilvadipine, nifedipine, teludipine hydrochloride, diltiazem hydrochloride (marketed as CARDIZEM®), belfosdil, verapamil hydrochloride (marketed as CALAN®), fostedil), nifedipine (marketed as ADALAT®), nicardipine (marketed as CARDENE®), nisoldipine (marketed as SULAR®), bepridil (marketed as VASCOR®); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride (marketed as LOTENSIN®), benazeprilat, captopril (marketed as CAPTOEN®), delapril hydrochloride, fosinopril sodium, libenzapril, moexipril hydrochloride (marketed as UNIVASC®), pentopril, perindopril, quinapril hydrochloride (marketed as ACCUPRIL®), quinaprilat, ramipril (marketed as RAMACE® and ALTACE®) (or ACE/NEP inhibitors such as ramipril, marketed as DELIX®/TRITACE®), spirapril hydrochloride, peridopril, (marketed as ACEON®), spiraprilat, trandolapil (marketed as MAVIK®), teprotide, enalapril maleate (marketed as VASOTEC®), lisinopril (marketed as ZESTRIL®), zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium (marketed as MONOPRIL®), guanfacine hydrochloride, lomerizine, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexyline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can comprise at least one compound of Formula (I), or pharmaceutically acceptable salts, solvates, isomers or esters thereof, and one or more antidiabetic medications for reducing blood glucose levels in a patient. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of two, three, four or more of any of the pharmacological or therapeutic agents described above can be used in the compositions and therapeutic combinations of the present invention.

Since the present invention relates to treating conditions as discussed above, by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one selective CB₁ receptor antagonist of Formula (I), or a pharmaceutically acceptable salt, solvate, or ester thereof, and a separate pharmaceutical composition comprising at least one cholesterol lowering compound as described above. The kit will preferably include directions for the administration of the separate components. The kit form is particularly advantageous when the separate components must be administered in different dosage forms (e.g., oral and parenteral) or are administered at different dosage intervals.

In yet another embodiment, the present invention provides a method of treating, reducing, or ameliorating a disease or condition selected from the group consisting of metabolic syndrome, obesity, waist circumference, abdominal girth, lipid profile, insulin sensitivity, neuroinflammatory disorders, cognitive disorders, psychosis, addictive behavior, gastrointestinal disorders, vascular conditions, hyperlipidaemia, atherosclerosis, hypercholesterolemia, sitosterolemia, vascular inflammation, stroke, diabetes, and cardiovascular conditions, and/or reduce the level of sterol(s) in a patient in need thereof, comprising administering to said patient an effective amount of at least one compound of Formula (I), or a pharmaceutically acceptable salt, solvate, or ester thereof, and one or more cholesterol lowering compound.

The treatment compositions and therapeutic combinations comprising at least one compound of Formula (I) and at least one cholesterol lowering agent can inhibit the intestinal absorption of cholesterol in mammals can be useful in the treatment and/or prevention of conditions, for example vascular conditions, such as atherosclerosis, hypercholesterolemia and sitosterolemia, stroke, obesity and lowering of plasma levels of cholesterol in mammals, in particular in mammals.

In another embodiment of the present invention, the compositions and therapeutic combinations of the present invention can inhibit sterol or 5α-stanol absorption or reduce plasma concentration of at least one sterol selected from the group consisting of phytosterols (such as sitosterol, campesterol, stigmasterol and avenosterol) and/or 5α-stanol (such as cholestanol, 5α-campestanol, 5α-sitostanol), cholesterol and mixtures thereof. The plasma concentration can be reduced by administering to a mammal in need of such treatment an effective amount of at least one treatment composition or therapeutic combination comprising at least one selective CB₁ receptor antagonist and at least one cholesterol lowering compound, for example a sterol absorption inhibitor described above. The reduction in plasma concentration of sterols or 5α-stanols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

The treatments of the present invention can also reduce the size or presence of plaque deposits in vascular vessels. The plaque volume can be measured using (IVUS), in which a tiny ultrasound probe is inserted into an artery to directly image and measure the size of atherosclerotic plaques, in a manner well known to those skilled in the art.

### EXAMPLES

### Preparation of Examples 1, 2, 4, 6, 7, 9, 19, and 10

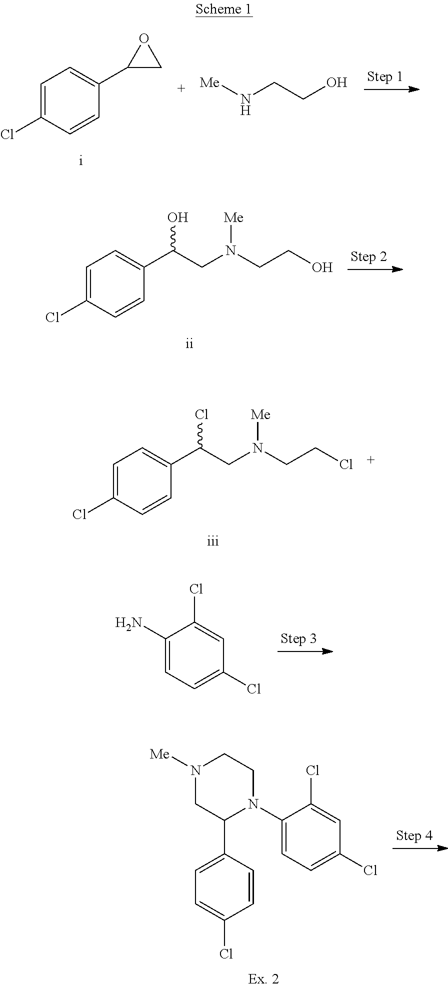 [SMILES: Clc1ccc(C2CO2)cc1 ; CNCCO ; CN(CCO)CC(O)c1ccc(Cl)cc1 ; CN(CCCl)CC(Cl)c1ccc(Cl)cc1 ; Nc1ccc(Cl)cc1Cl ; CN1CCN(c2ccc(Cl)cc2Cl)C(c2ccc(Cl)cc2)C1]

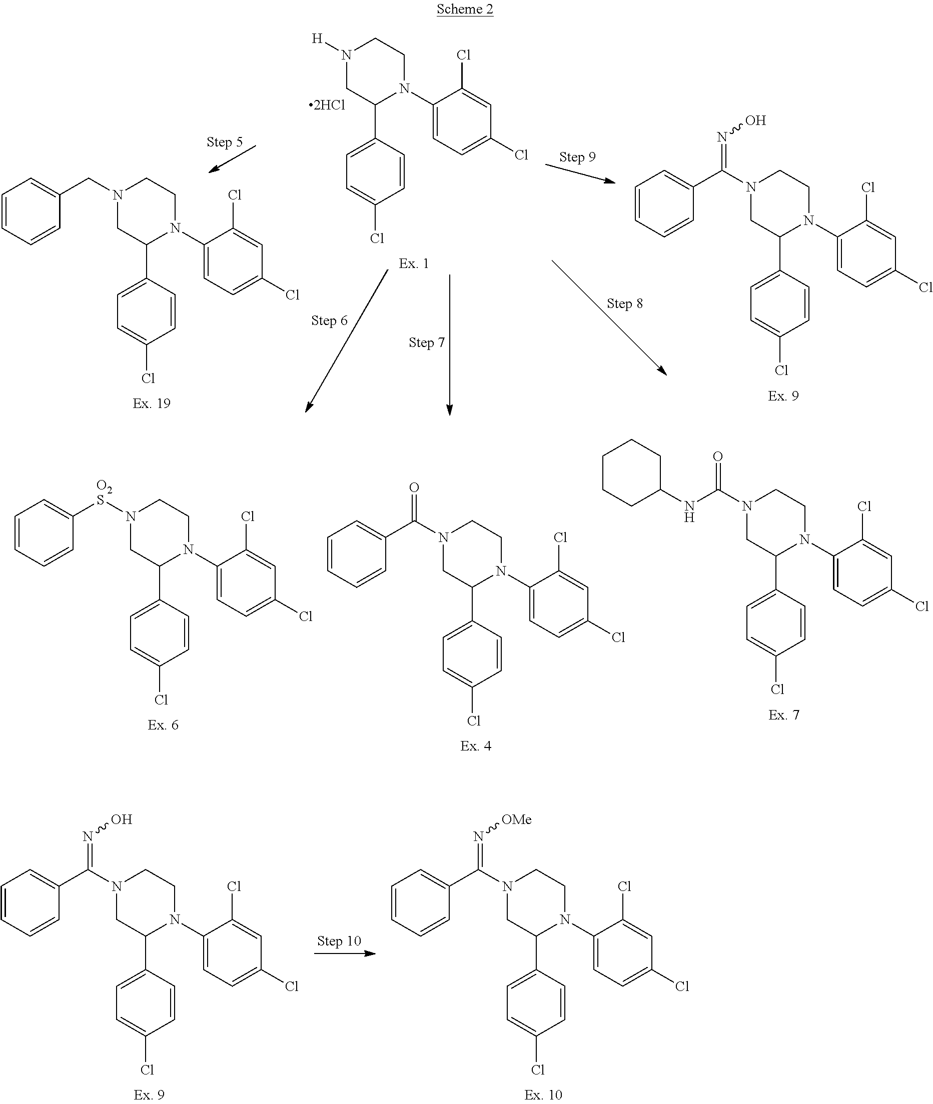

### Step 1:

To neat 2-(4-chlorophenyl)oxirane i (10.1 g, 65.4 mmol) was added N-methylethanolamine (7.36 g, 98.1 mmol). The reaction mixture was warmed to 130° C. and stirred for 15 h. The reaction mixture was then cooled to room temperature (approximately 21° C.) and purified directly by silica gel chromatography (8% MeOH/CH₂Cl₂) to provide the diol ii (13.1 g, 57.2 mmol).

### Step 2:

To a solution of the diol ii (13.1 g, 57.2 mmol) in CHCl₃ (110 mL) at 0° C. was added a solution of SOCl₂ (57 mL) in CHCl₃ (100 mL), dropwise, over 20 minutes. After the addition of the SOCl₂ solution was complete, the reaction mixture was warmed to reflux and stirred for 3.5 h. The reaction was cooled to room temperature and then concentrated in vacuo. The resulting oil was taken up into CH₂Cl₂ and stirred vigorously with saturated aqueous NaHCO₃. The mixture was extracted with CH₂Cl₂ and the combined CH₂Cl₂ layers were washed with water and brine, then dried (MgSO₄), filtered, and concentrated in vacuo to provide iii (14.2 g, 53.2 mmol). The chloride iii was used directly, or converted to its HCl salt. The HCl salt of chloride iii was prepared by dissolving chloride iii in CH₂Cl₂ and adding excess 2N HCl/diethyl ether. After stirring the mixture for 5 minutes, the solvent was removed in vacuo to provide the HCl salt of chloride iii as a solid.

### Step 3:

To a solution of iii (3.27 g, 12.3 mmol) in propionitrile (40 mL) was added 2,4-dichloroaniline (5.97 g, 36.9 mmol). The reaction mixture was warmed to reflux and stirred for 18 h. The reaction mixture was then cooled to room temperature and concentrated in vacuo. Purification of the residue by silica gel chromatography (5% MeOH/CH₂Cl₂) provided the piperazine Example 2 (2.92 g, 8.21 mmol).

### Step 4:

To a solution of the piperazine Example 2 (2.92 g, 8.21 mmol) in dichloroethane (27 mL) at room temperature was added Proton-Sponge® (1,8-bis(dimethylamino)naphthalene; available from Aldrich) (0.52 g, 2.46 mmol) and 1-chloroethylchloroformate (2.35 g, 16.4 mmol). The reaction mixture was warmed to reflux and stirred for 21 h. The dichloroethane was removed in vacuo and MeOH (30 mL) was then added. The reaction mixture was then warmed to reflux and stirred for 7 h. The reaction mixture was concentrated in vacuo and the resulting oil was taken up into CH₂Cl₂. Saturated aqueous NaHCO₃ was added, and the mixture was stirred vigorously, then extracted with CH₂Cl₂. The organic layers were combined and washed with water and brine, dried (MgSO₄), filtered, and concentrated in vacuo to provide the piperazine Example 1 (2.11 g, 6.19 mmol).

### Step 5:

To a solution of piperazine Example 1 (0.135 g, 0.33 mmol) in dichloroethane (1.3 mL) was added triethylamine (0.07 g, 0.66 mmol), benzaldehyde (0.052 g, 0.49 mmol), and sodium triacetoxyborohydride (0.14 g, 0.66 mmol). The reaction was stirred for 18 h at room temperature. CH₂Cl₂ was added, and the solution was then washed with saturated aqueous NaHCO₃, water, and brine, dried (MgSO₄), filtered, and concentrated in vacuo to provide a residue that was adsorbed on excess PS-TsOH resin in CH₂Cl₂. After 2 h, the resin was filtered and washed with CH₂Cl₂ and MeOH. The resin was then stirred with 7N NH₃/MeOH for 1 h, filtered, and washed with CH₂Cl₂. The filtrate was concentrated in vacuo to provide Example 19 (0.086 g, 0.20 mmol).

### Step 6:

To a solution of the piperazine Example 1 (0.10 g, 0.24 mmol) in dichloroethane (1 mL) was added TEA (i.e., triethylamine) (0.09 g, 0.96 mmol) and benzenesulfonyl chloride (0.05 g, 0.3 mmol) at room temperature. The reaction mixture was stirred for 12 h and concentrated in vacuo. The resulting residue was purified by silica gel preparative plate TLC (i.e., thin layer chromatography) (2000 μm, 20% EtOAc/hexane) to provide the sulfonamide Example 6 (0.11 g, 0.22 mmol).

### Step 7:

To a solution of the piperazine Example 1 (0.14 g, 0.33 mmol) in CH₂Cl₂ (1.7 mL) at 0° C. was added TEA (0.10 g, 0.10 mmol) followed by benzoyl chloride (0.05 g, 0.37 mmol). The cold bath was allowed to warm slowly to room temperature, and the reaction mixture was stirred for 20 h. CH₂Cl₂ was added and the mixture washed with water and brine, dried (MgSO₄), filtered, and concentrated in vacuo. The resulting residue was purified by silica gel chromatography to provide the amide Example 4 (0.13 g, 0.29 g).

### Step 8:

To a solution of the piperazine Example 1 (0.18 g, 0.45 mmol) in dichloroethane (2 mL) was added TEA (0.14 g, 1.3 mmol) followed by cyclohexylisocyanate (0.08 g, 0.67 mmol). The reaction mixture was warmed to reflux and stirred for 17 h, then cooled to room temperature. CH₂Cl₂ was added, and the solution was washed with water and brine, dried (MgSO₄), filtered and concentrated in vacuo. The resulting residue was purified by silica gel prep plate TLC (2000 μm, 30% EtOAc/hexane) to provide the urea Example 7 (0.19 g, 0.41 mmol).

### Step 9:

To a solution of benzaldehyde (2.00 g, 18.8 mmol) in ethanol (13 mL) was added hydroxylamine hydrochloride (2.61 g, 37.6 mmol) and pyridine (3.8 mmol). The solution was warmed to reflux and stirred for 18 h. The reaction mixture was concentrated in vacuo and the resulting residue was taken up into CH₂Cl₂, washed with water and brine, dried (MgSO₄), filtered, and concentrated in vacuo to provide the phenyloxime (2.0 g, 16.5 mmol). The phenyloxime was taken up into DMF (i.e., dimethylformamide) (55 mL), and N-chlorosuccinimide (2.43 g, 18.2 mmol) was added at room temperature. The reaction mixture was stirred for 23 h and then water was added. The mixture was extracted with EtOAc. The organic layers were combined and washed with water and brine, dried (MgSO₄), filtered and concentrated in vacuo to provide the benzohydroximoyl chloride that was used directly.

To the piperazine Example 1 (0.50 g, 1.21 mmol) in CH₂Cl₂ (4 mL) was added diisopropylethylamine (0.55 g, 4.23 mmol) and the benzohydroximoyl chloride prepared as described above (0.28 g, 1.81 mmol), at room temperature. The reaction mixture was stirred for 15 h and CH₂Cl₂ was then added. The solution was then washed with water and brine, dried (MgSO₄), filtered and concentrated in vacuo. The residue was purified by silica gel chromatography (30% EtOAc/hexane) to provide the amidoxime Example 9 (0.44 g, 0.95 mmol).

### Step 10:

To a solution of the amidoxime Example 9 (0.074 g, 0.15 mmol) in toluene (0.5 mL) was added 50% aqueous NaOH (0.5 mL), methyl iodide (0.04 g, 0.30 mmol), and tetrabutylammonium iodide (0.003 g, 0.007 mmol). The resulting reaction mixture was stirred at room temperature for 18 h, then water was added and the mixture was extracted with EtOAc. The combined organic layers were washed with water and brine, dried (MgSO₄), filtered, and concentrated in vacuo. The resulting residue was purified by silica gel preparative plate TLC (2000 μm, 10% EtOAc/hexane) to provide the O-methylamidoxime Example 10 (0.05 g, 0.10 mmol).

### Preparation of Example 3

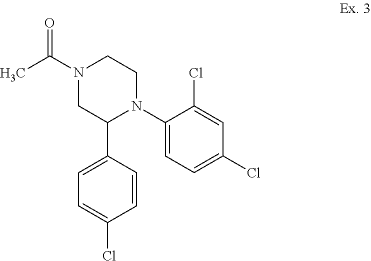 [SMILES: CC(=O)N1CCN(c2ccc(Cl)cc2Cl)C(c2ccc(Cl)cc2)C1]

Example 3 was prepared from Example 1 using procedures similar to those used to prepare Example 4, except that acetyl chloride was used in Step 7 (above) instead of benzoyl chloride.

### Preparation of Example 5

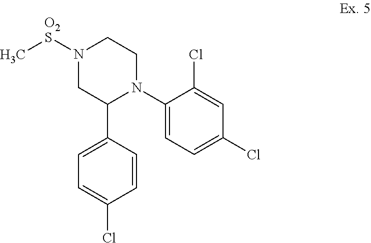 [SMILES: CS(=O)(=O)N1CCN(c2ccc(Cl)cc2Cl)C(c2ccc(Cl)cc2)C1]

Example 5 was prepared from Example 1 using procedures similar to those used to prepare Example 6, except that methanesulfonyl chloride was used in Step 6 (above) instead of benzenesulfonyl chloride.

### Preparation of Example 8

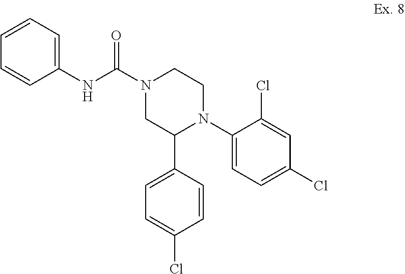 [SMILES: O=C(Nc1ccccc1)N1CCN(c2ccc(Cl)cc2Cl)C(c2ccc(Cl)cc2)C1]

Example 8 was prepared from Example 1 using procedures similar to those used to prepare Example 7, except that phenylisocyanate was used in Step 8 (above) instead of cyclohexylisocyanate.

### Preparation of Example 11

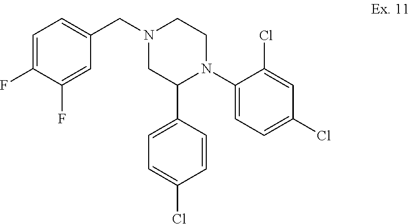 [SMILES: Fc1ccc(CN2CCN(c3ccc(Cl)cc3Cl)C(c3ccc(Cl)cc3)C2)cc1F]

Example 11 was prepared from Example 1 using procedures similar to those used to prepare Example 19, except that 3,4-difluorobenzaldehyde was used in Scheme 2, Step 5 (above) instead of benzaldehyde.

### Preparation of Example 12

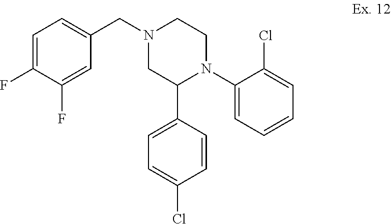 [SMILES: Fc1ccc(CN2CCN(c3ccccc3Cl)C(c3ccc(Cl)cc3)C2)cc1F]

Example 12 was prepared using procedures similar to those used to prepare Example 19, except that in Scheme 1, Step 3 (above), the HCl salt of iii was used instead of iii, 2-chloroaniline was used instead of 2,4-dichloroaniline, acetonitrile was used instead of propionitrile, NaI (1 equiv.) and diisopropylethylamine (3 equiv.) was added, and in Scheme 2, Step 5 (above), 3,4-difluorobenzaldehyde was used instead of benzaldehyde.

### Preparation of Example 13

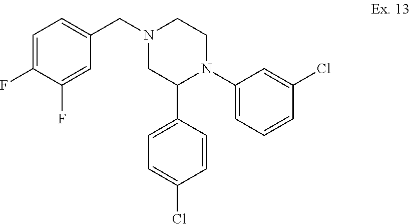 [SMILES: Fc1ccc(CN2CCN(c3cccc(Cl)c3)C(c3ccc(Cl)cc3)C2)cc1F]

Example 13 was prepared using procedures similar to those used to prepare Example 19, except that in Scheme 1, Step 3 (above), the HCl salt of iii was used instead of iii, 3-chloroaniline was used instead of 2,4-dichloroaniline, acetonitrile was used instead of propionitrile, NaI (1 equiv.) and diisopropylethylamine (3 equiv.) was added, and in Scheme 2, Step 5 (above), 3,4-difluorobenzaldehyde was used instead of benzaldehyde.

### Preparation of Example 14

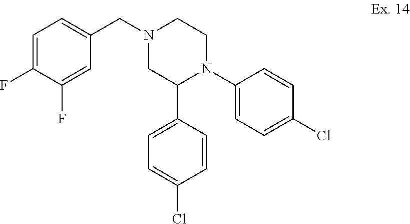 [SMILES: Fc1ccc(CN2CCN(c3ccc(Cl)cc3)C(c3ccc(Cl)cc3)C2)cc1F]

Example 14 was prepared using procedures similar to those used to prepare Example 19, except that in Scheme 1, Step 3 (above), 4-chloroaniline was used instead of 2,4-dichloroaniline, acetonitrile was used instead of propionitrile, and NaI was added, and in Scheme 2, Step 5 (above), 3,4-difluorobenzaldehyde was used instead of benzaldehyde.

### Preparation of Example 15

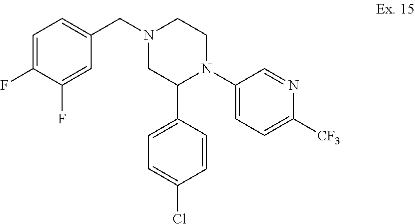 [SMILES: Fc1ccc(CN2CCN(c3ccc(C(F)(F)F)nc3)C(c3ccc(Cl)cc3)C2)cc1F]

Example 15 was prepared using procedures similar to those used to prepare Example 19, except that in Scheme 1, Step 3 (above), 6-trifluoromethyl-pyridin-3-ylamine was used instead of 2,4-dichloroaniline, acetonitrile was used instead of propionitrile, NaI (1 equiv.) and diisopropylethylamine (3 equiv.) was added, and in Scheme 2, Step 5 (above), 3,4-difluorobenzaldehyde was used instead of benzaldehyde.

### Preparation of Example 16

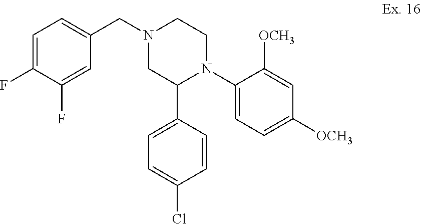 [SMILES: COc1ccc(N2CCN(Cc3ccc(F)c(F)c3)CC2c2ccc(Cl)cc2)c(OC)c1]

Example 16 was prepared using procedures similar to those used to prepare Example 19, except that in Scheme 1, Step 3 (above), 2,4-dimethoxyaniline was used instead of 2,4-dichloroaniline, acetonitrile was used instead of propionitrile, NaI (1 equiv.) and diisopropylethylamine (3 equiv.) was added, and in Scheme 2, Step 5 (above), 3,4-difluorobenzaldehyde was used instead of benzaldehyde.

### Preparation of Example 17

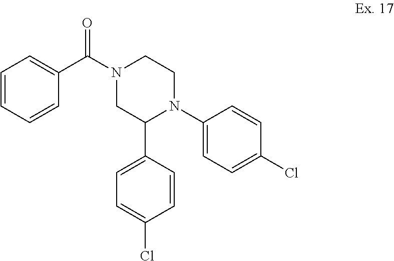 [SMILES: O=C(c1ccccc1)N1CCN(c2ccc(Cl)cc2)C(c2ccc(Cl)cc2)C1]

Example 17 was prepared using procedures similar to those used to prepare Example 4, except that in Scheme 1, Step 3 (above), the HCl salt of iii was used instead of iii, 4-chloroaniline was used instead of 2,4-dichloroaniline, acetonitrile was used instead of propionitrile, and NaI (1 equiv.) and diisopropylethylamine (3 equiv.) was added.

### Preparation of Example 18

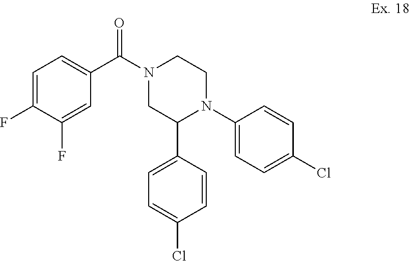 [SMILES: O=C(c1ccc(F)c(F)c1)N1CCN(c2ccc(Cl)cc2)C(c2ccc(Cl)cc2)C1]

Example 18 was prepared using procedures similar to those used to prepare Example 4, except that in Scheme 1, Step 3 (above), the HCl salt of iii was used instead of iii, 4-chloroaniline was used instead of 2,4-dichloroaniline, acetonitrile was used instead of propionitrile, NaI (1 equiv.) and diisopropylethylamine (3 equiv.) was added, and in Step 7 (above), 3,4-difluorobenzoyl chloride was used instead of benzoyl chloride.

### Preparation of Examples 20-110

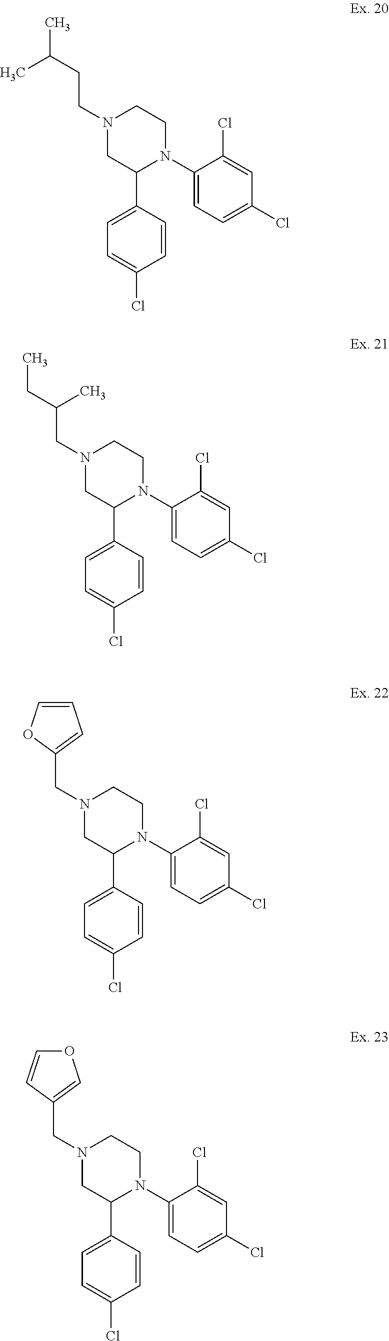 [SMILES: CC(C)CCN1CCN(c2ccc(Cl)cc2Cl)C(c2ccc(Cl)cc2)C1 ; CCC(C)CN1CCN(c2ccc(Cl)cc2Cl)C(c2ccc(Cl)cc2)C1 ; Clc1ccc(C2CN(Cc3ccco3)CCN2c2ccc(Cl)cc2Cl)cc1 ; Clc1ccc(C2CN(Cc3ccoc3)CCN2c2ccc(Cl)cc2Cl)cc1]

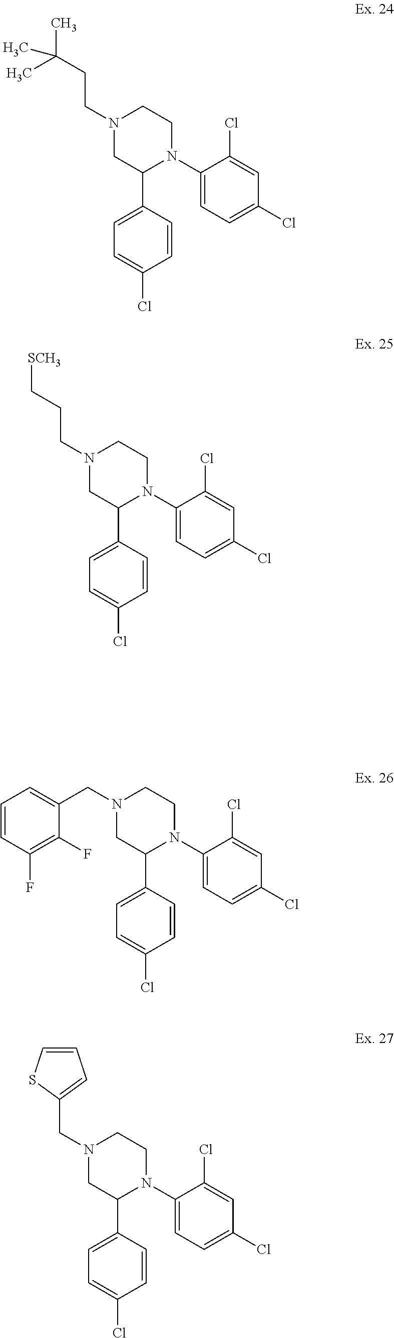 [SMILES: CC(C)(C)CCN1CCN(c2ccc(Cl)cc2Cl)C(c2ccc(Cl)cc2)C1 ; CSCCCN1CCN(c2ccc(Cl)cc2Cl)C(c2ccc(Cl)cc2)C1 ; Fc1cccc(CN2CCN(c3ccc(Cl)cc3Cl)C(c3ccc(Cl)cc3)C2)c1F ; Clc1ccc(C2CN(Cc3cccs3)CCN2c2ccc(Cl)cc2Cl)cc1]

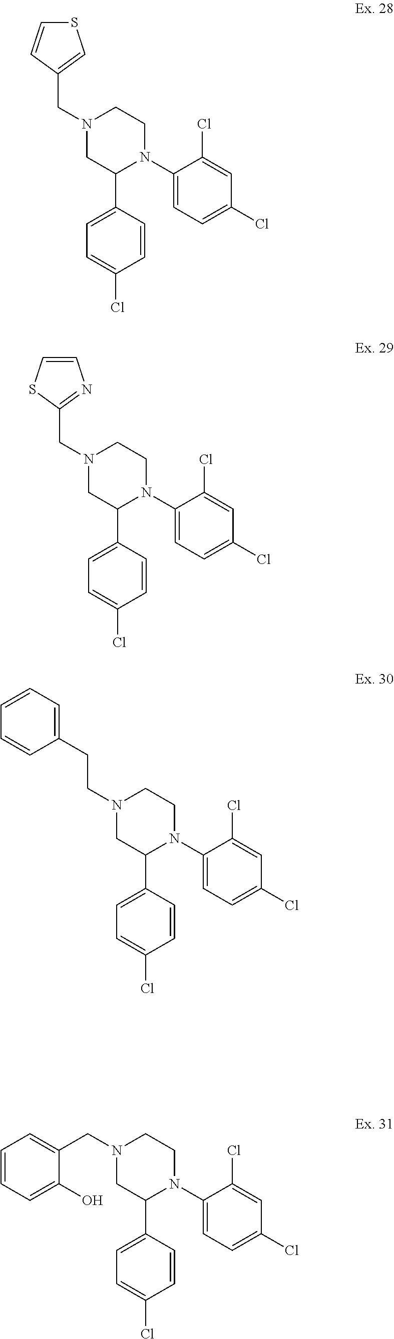 [SMILES: Clc1ccc(C2CN(Cc3ccsc3)CCN2c2ccc(Cl)cc2Cl)cc1 ; Clc1ccc(C2CN(Cc3nccs3)CCN2c2ccc(Cl)cc2Cl)cc1 ; Clc1ccc(C2CN(CCc3ccccc3)CCN2c2ccc(Cl)cc2Cl)cc1 ; Oc1ccccc1CN1CCN(c2ccc(Cl)cc2Cl)C(c2ccc(Cl)cc2)C1]

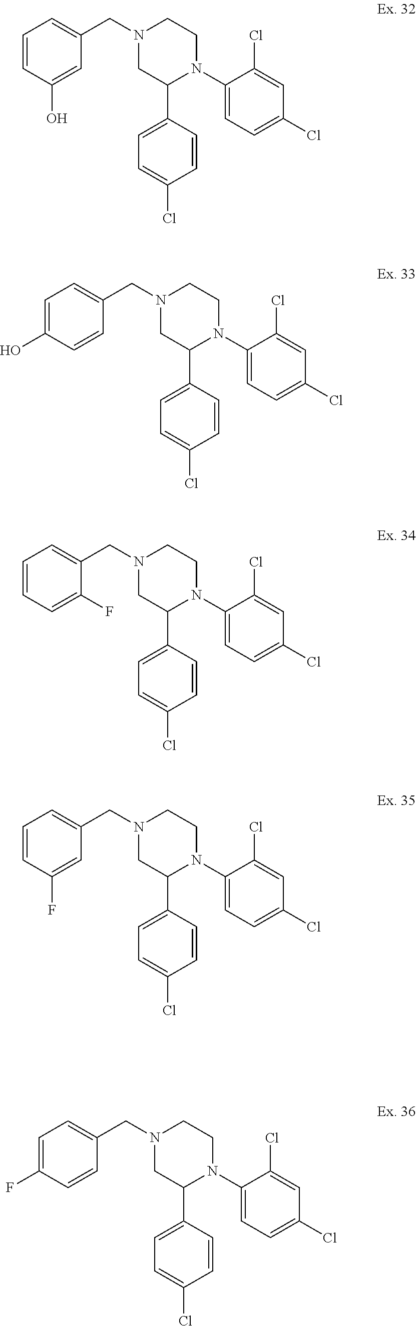 [SMILES: Oc1cccc(CN2CCN(c3ccc(Cl)cc3Cl)C(c3ccc(Cl)cc3)C2)c1 ; Oc1ccc(CN2CCN(c3ccc(Cl)cc3Cl)C(c3ccc(Cl)cc3)C2)cc1 ; Fc1ccccc1CN1CCN(c2ccc(Cl)cc2Cl)C(c2ccc(Cl)cc2)C1 ; Fc1cccc(CN2CCN(c3ccc(Cl)cc3Cl)C(c3ccc(Cl)cc3)C2)c1 ; Fc1ccc(CN2CCN(c3ccc(Cl)cc3Cl)C(c3ccc(Cl)cc3)C2)cc1]

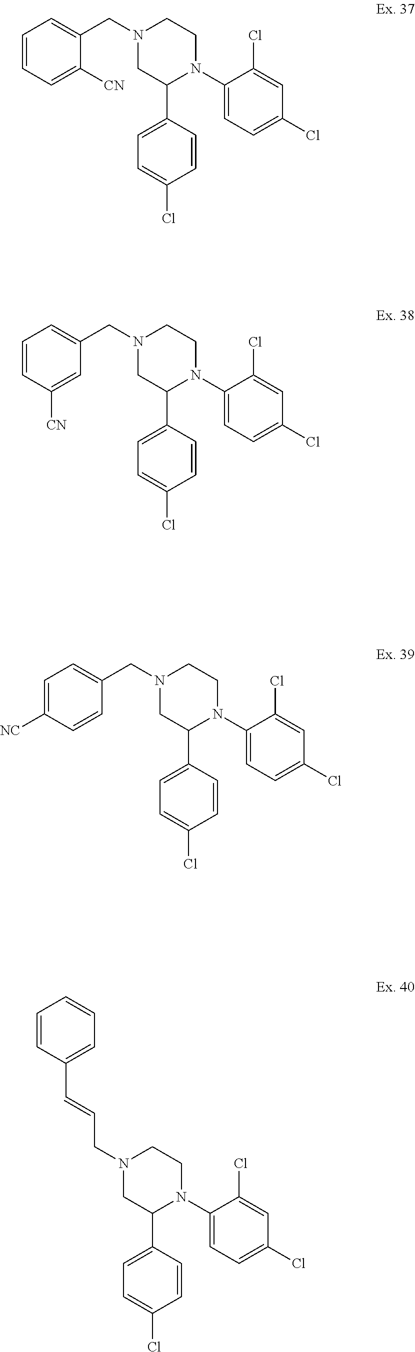 [SMILES: N#Cc1ccccc1CN1CCN(c2ccc(Cl)cc2Cl)C(c2ccc(Cl)cc2)C1 ; N#Cc1cccc(CN2CCN(c3ccc(Cl)cc3Cl)C(c3ccc(Cl)cc3)C2)c1 ; N#Cc1ccc(CN2CCN(c3ccc(Cl)cc3Cl)C(c3ccc(Cl)cc3)C2)cc1 ; Clc1ccc(C2CN(C/C=C/c3ccccc3)CCN2c2ccc(Cl)cc2Cl)cc1]

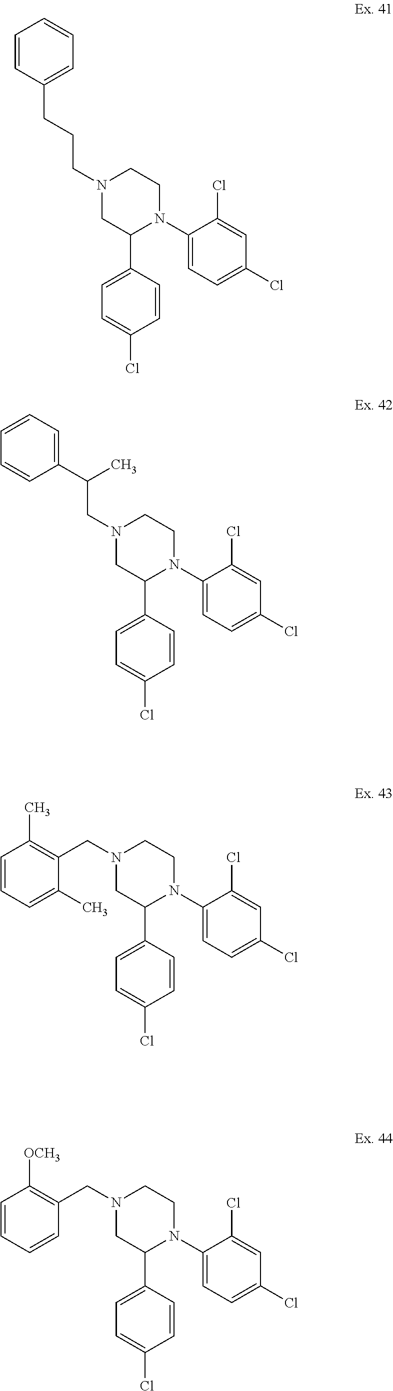 [SMILES: Clc1ccc(C2CN(CCCc3ccccc3)CCN2c2ccc(Cl)cc2Cl)cc1 ; CC(CN1CCN(c2ccc(Cl)cc2Cl)C(c2ccc(Cl)cc2)C1)c1ccccc1 ; Cc1cccc(C)c1CN1CCN(c2ccc(Cl)cc2Cl)C(c2ccc(Cl)cc2)C1 ; COc1ccccc1CN1CCN(c2ccc(Cl)cc2Cl)C(c2ccc(Cl)cc2)C1]

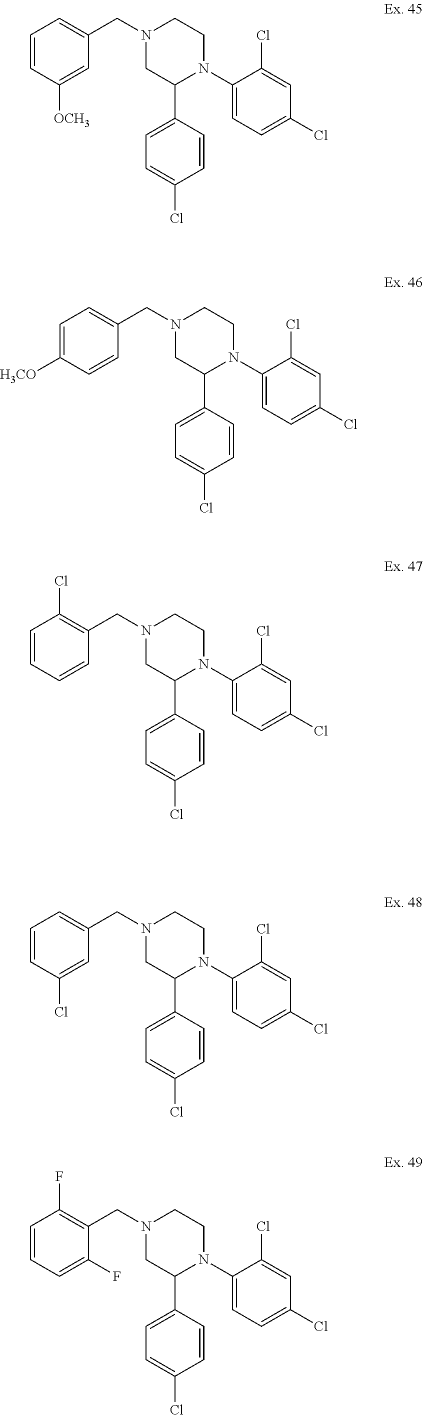 [SMILES: COc1cccc(CN2CCN(c3ccc(Cl)cc3Cl)C(c3ccc(Cl)cc3)C2)c1 ; COc1ccc(CN2CCN(c3ccc(Cl)cc3Cl)C(c3ccc(Cl)cc3)C2)cc1 ; Clc1ccc(C2CN(Cc3ccccc3Cl)CCN2c2ccc(Cl)cc2Cl)cc1 ; Clc1ccc(C2CN(Cc3cccc(Cl)c3)CCN2c2ccc(Cl)cc2Cl)cc1 ; Fc1cccc(F)c1CN1CCN(c2ccc(Cl)cc2Cl)C(c2ccc(Cl)cc2)C1]

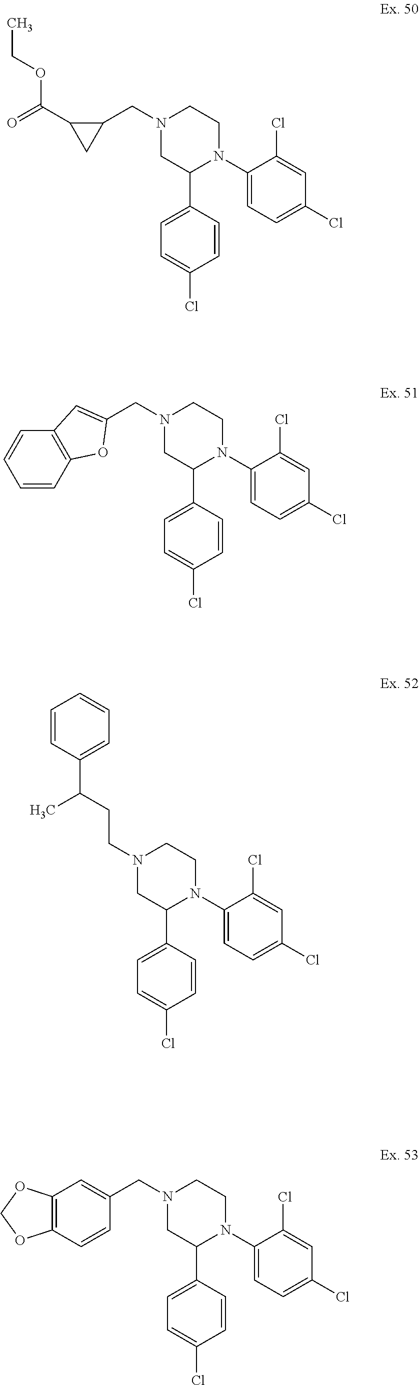 [SMILES: CCOC(=O)C1CC1CN1CCN(c2ccc(Cl)cc2Cl)C(c2ccc(Cl)cc2)C1 ; Clc1ccc(C2CN(Cc3cc4ccccc4o3)CCN2c2ccc(Cl)cc2Cl)cc1 ; CC(CCN1CCN(c2ccc(Cl)cc2Cl)C(c2ccc(Cl)cc2)C1)c1ccccc1 ; Clc1ccc(C2CN(Cc3ccc4c(c3)OCO4)CCN2c2ccc(Cl)cc2Cl)cc1]

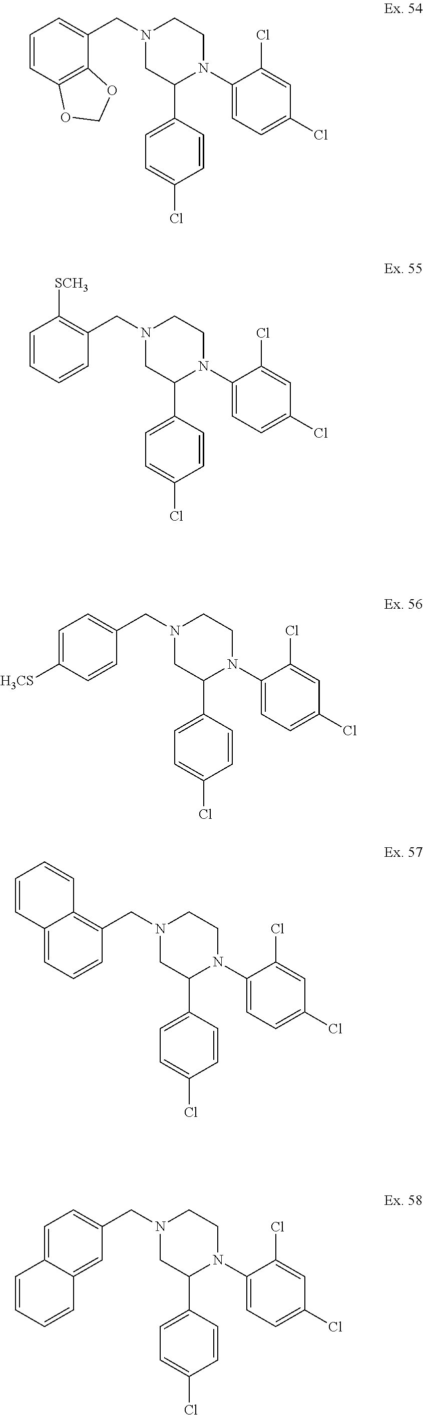

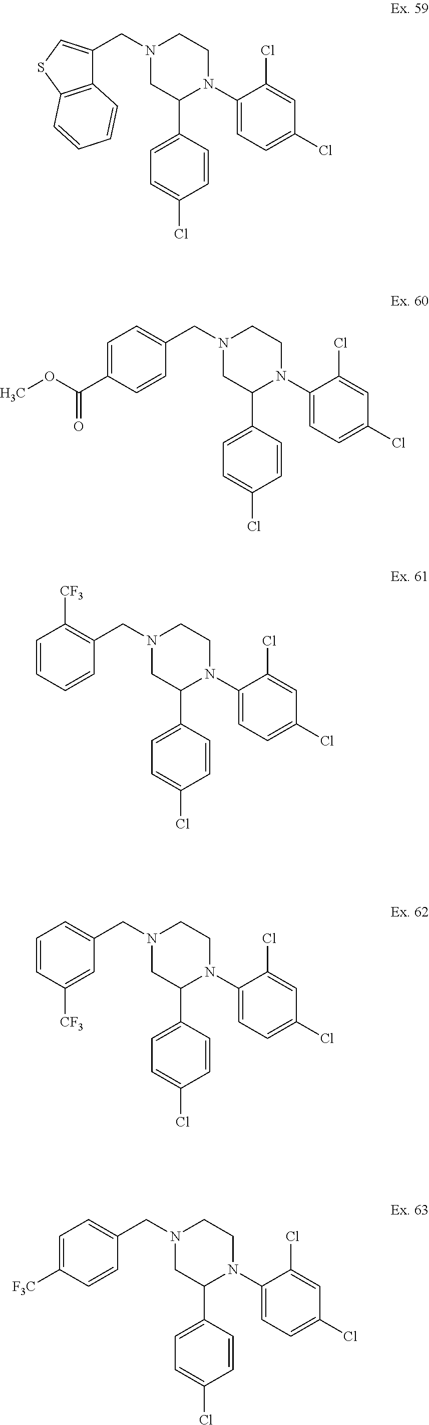 [SMILES: Clc1ccc(C2CN(Cc3csc4ccccc34)CCN2c2ccc(Cl)cc2Cl)cc1 ; COC(=O)c1ccc(CN2CCN(c3ccc(Cl)cc3Cl)C(c3ccc(Cl)cc3)C2)cc1 ; FC(F)(F)c1ccccc1CN1CCN(c2ccc(Cl)cc2Cl)C(c2ccc(Cl)cc2)C1 ; FC(F)(F)c1cccc(CN2CCN(c3ccc(Cl)cc3Cl)C(c3ccc(Cl)cc3)C2)c1 ; FC(F)(F)c1ccc(CN2CCN(c3ccc(Cl)cc3Cl)C(c3ccc(Cl)cc3)C2)cc1]

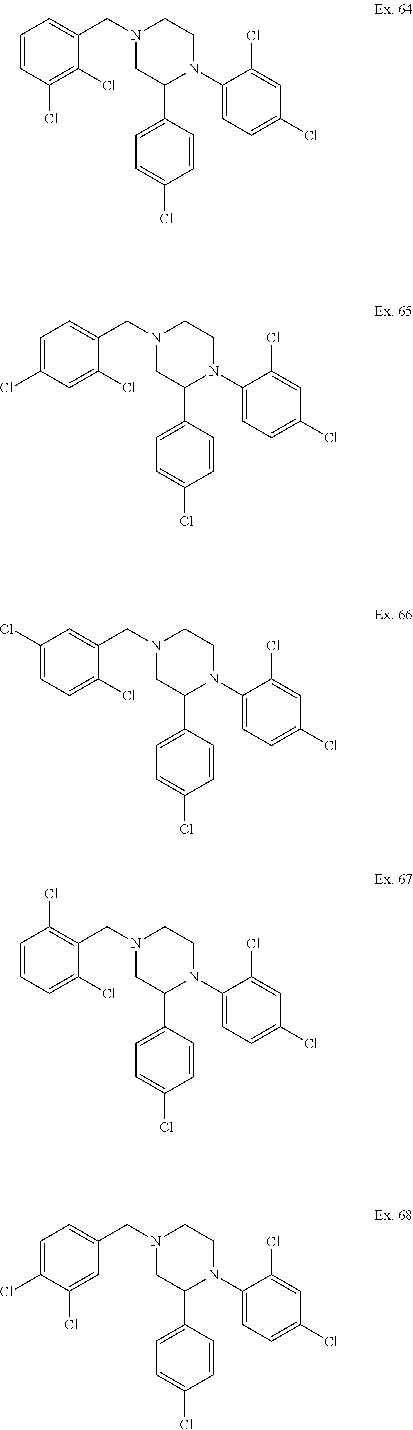 [SMILES: Clc1ccc(C2CN(Cc3cccc(Cl)c3Cl)CCN2c2ccc(Cl)cc2Cl)cc1 ; Clc1ccc(C2CN(Cc3ccc(Cl)cc3Cl)CCN2c2ccc(Cl)cc2Cl)cc1 ; Clc1ccc(C2CN(Cc3cc(Cl)ccc3Cl)CCN2c2ccc(Cl)cc2Cl)cc1 ; Clc1ccc(C2CN(Cc3c(Cl)cccc3Cl)CCN2c2ccc(Cl)cc2Cl)cc1 ; Clc1ccc(C2CN(Cc3ccc(Cl)c(Cl)c3)CCN2c2ccc(Cl)cc2Cl)cc1]

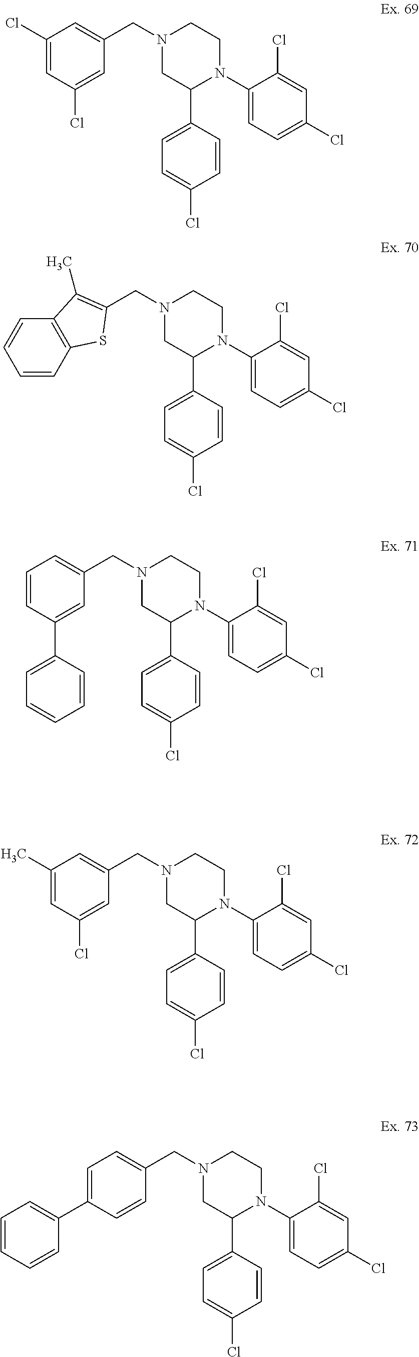 [SMILES: Clc1ccc(C2CN(Cc3cc(Cl)cc(Cl)c3)CCN2c2ccc(Cl)cc2Cl)cc1 ; Cc1c(CN2CCN(c3ccc(Cl)cc3Cl)C(c3ccc(Cl)cc3)C2)sc2ccccc12 ; Clc1ccc(C2CN(Cc3cccc(-c4ccccc4)c3)CCN2c2ccc(Cl)cc2Cl)cc1 ; Cc1cc(Cl)cc(CN2CCN(c3ccc(Cl)cc3Cl)C(c3ccc(Cl)cc3)C2)c1 ; Clc1ccc(C2CN(Cc3ccc(-c4ccccc4)cc3)CCN2c2ccc(Cl)cc2Cl)cc1]

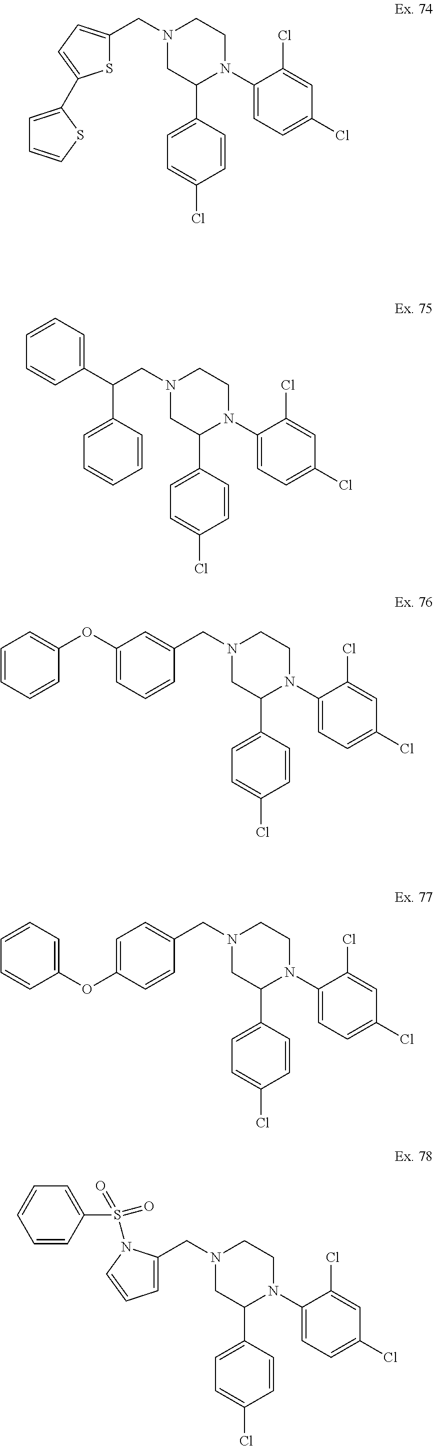 [SMILES: Clc1ccc(C2CN(Cc3ccc(-c4cccs4)s3)CCN2c2ccc(Cl)cc2Cl)cc1 ; Clc1ccc(C2CN(CC(c3ccccc3)c3ccccc3)CCN2c2ccc(Cl)cc2Cl)cc1 ; Clc1ccc(C2CN(Cc3cccc(Oc4ccccc4)c3)CCN2c2ccc(Cl)cc2Cl)cc1 ; Clc1ccc(C2CN(Cc3ccc(Oc4ccccc4)cc3)CCN2c2ccc(Cl)cc2Cl)cc1 ; O=S(=O)(c1ccccc1)n1cccc1CN1CCN(c2ccc(Cl)cc2Cl)C(c2ccc(Cl)cc2)C1]

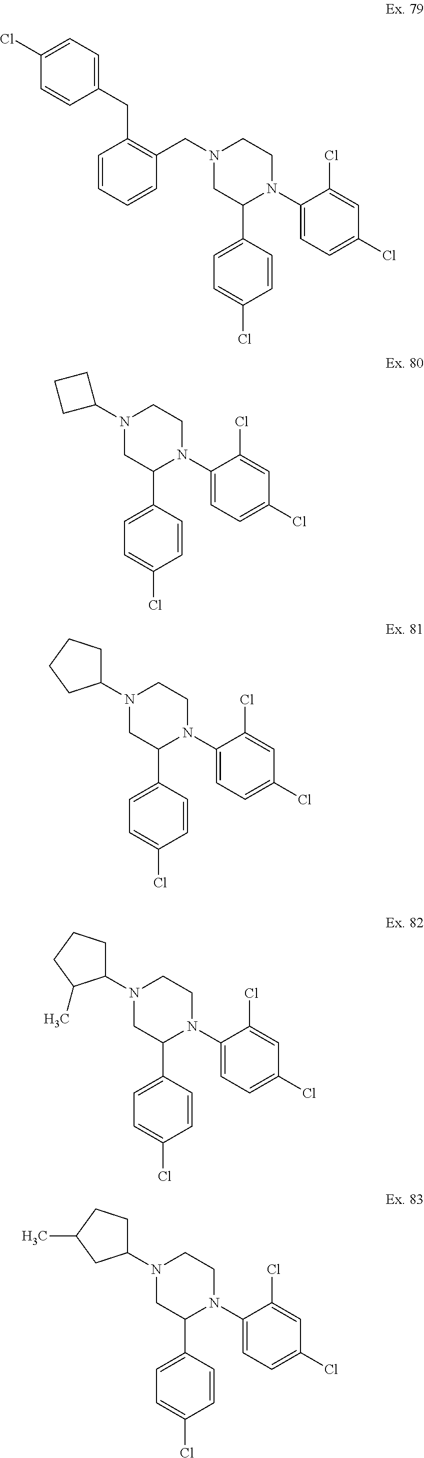 [SMILES: Clc1ccc(Cc2ccccc2CN2CCN(c3ccc(Cl)cc3Cl)C(c3ccc(Cl)cc3)C2)cc1 ; Clc1ccc(C2CN(C3CCC3)CCN2c2ccc(Cl)cc2Cl)cc1 ; Clc1ccc(C2CN(C3CCCC3)CCN2c2ccc(Cl)cc2Cl)cc1 ; CC1CCCC1N1CCN(c2ccc(Cl)cc2Cl)C(c2ccc(Cl)cc2)C1 ; CC1CCC(N2CCN(c3ccc(Cl)cc3Cl)C(c3ccc(Cl)cc3)C2)C1]

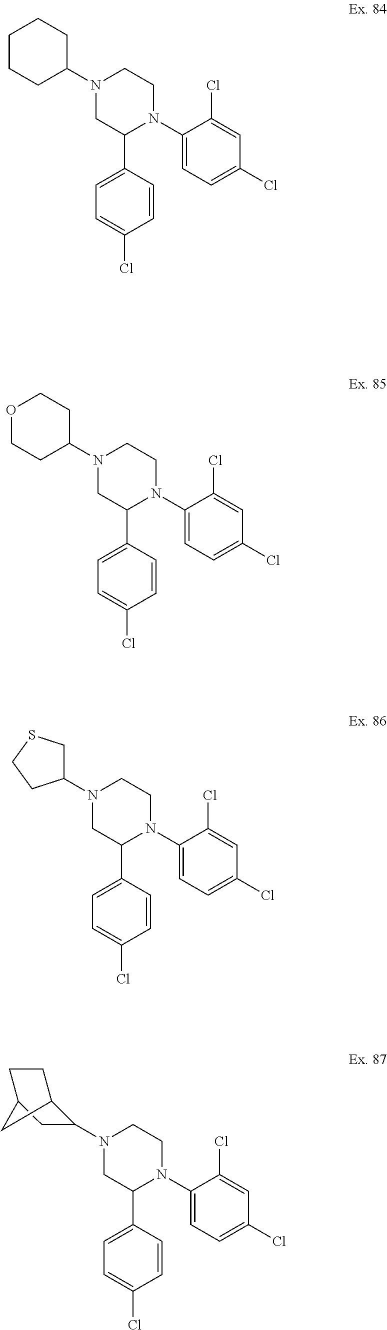 [SMILES: Clc1ccc(C2CN(C3CCCCC3)CCN2c2ccc(Cl)cc2Cl)cc1 ; Clc1ccc(C2CN(C3CCOCC3)CCN2c2ccc(Cl)cc2Cl)cc1 ; Clc1ccc(C2CN(C3CCSC3)CCN2c2ccc(Cl)cc2Cl)cc1 ; Clc1ccc(C2CN(C3CC4CCC3C4)CCN2c2ccc(Cl)cc2Cl)cc1]

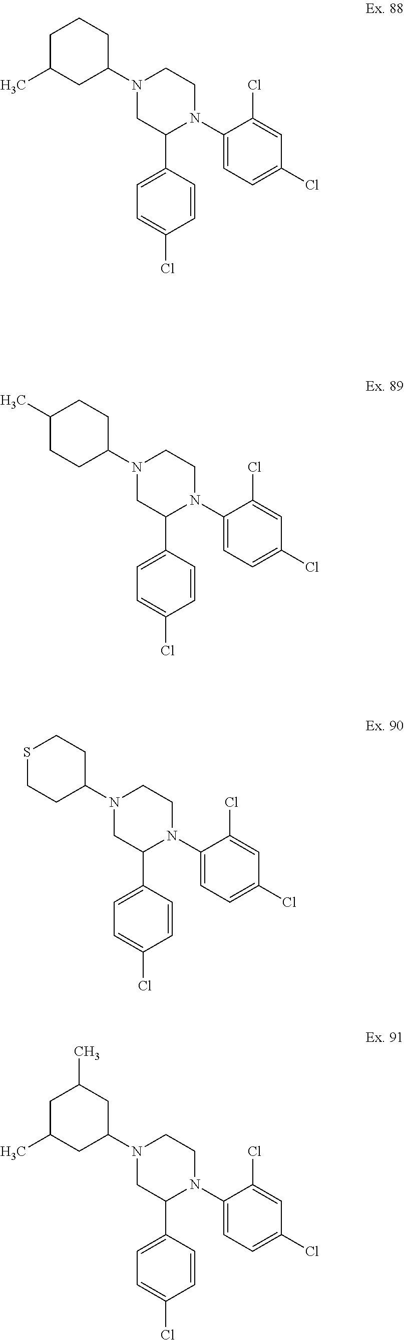 [SMILES: CC1CCCC(N2CCN(c3ccc(Cl)cc3Cl)C(c3ccc(Cl)cc3)C2)C1 ; CC1CCC(N2CCN(c3ccc(Cl)cc3Cl)C(c3ccc(Cl)cc3)C2)CC1 ; Clc1ccc(C2CN(C3CCSCC3)CCN2c2ccc(Cl)cc2Cl)cc1 ; CC1CC(C)CC(N2CCN(c3ccc(Cl)cc3Cl)C(c3ccc(Cl)cc3)C2)C1]

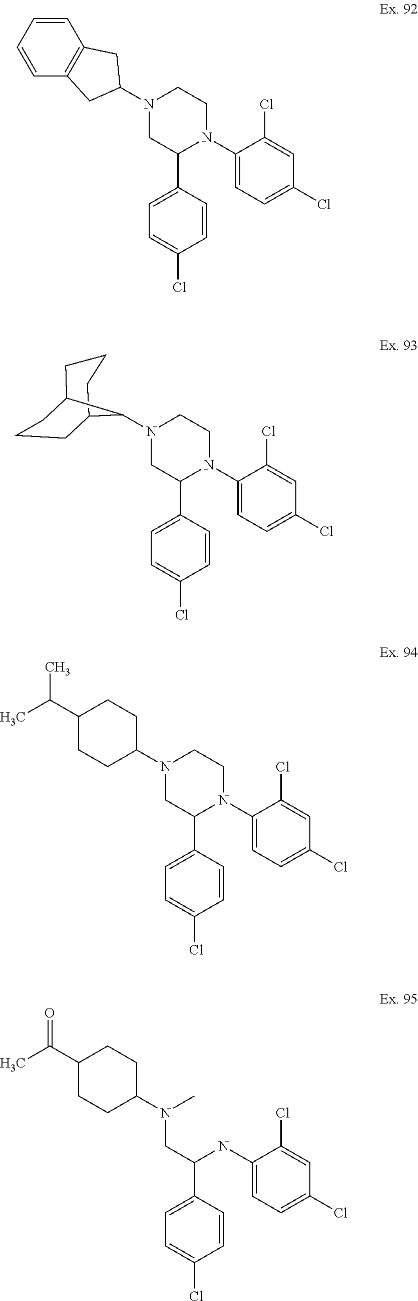 [SMILES: Clc1ccc(C2CN(C3Cc4ccccc4C3)CCN2c2ccc(Cl)cc2Cl)cc1 ; Clc1ccc(C2CN(C3C4CCCC3CCC4)CCN2c2ccc(Cl)cc2Cl)cc1 ; CC(C)C1CCC(N2CCN(c3ccc(Cl)cc3Cl)C(c3ccc(Cl)cc3)C2)CC1 ; CC(=O)C1CCC(N(C)CC(Nc2ccc(Cl)cc2Cl)c2ccc(Cl)cc2)CC1]

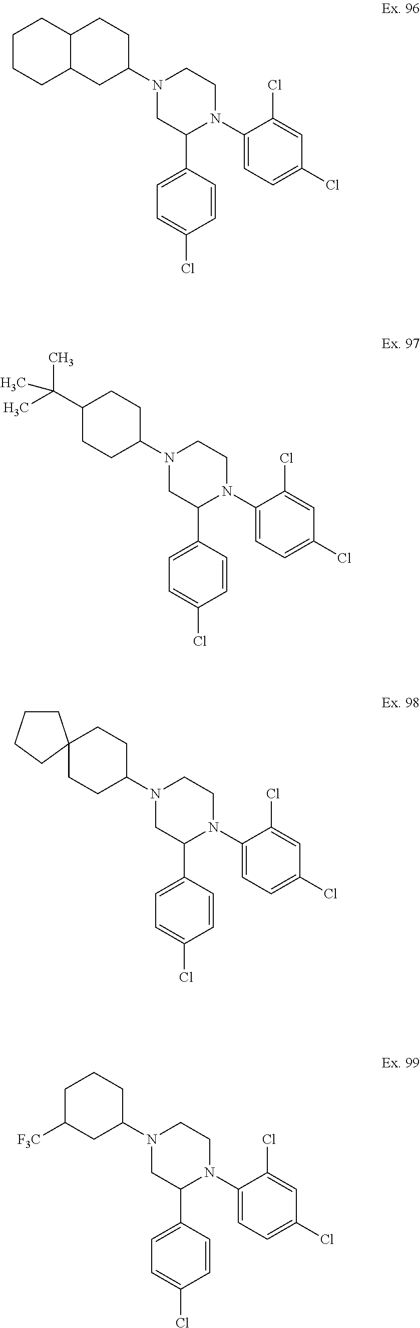 [SMILES: Clc1ccc(C2CN(C3CCC4CCCCC4C3)CCN2c2ccc(Cl)cc2Cl)cc1 ; CC(C)(C)C1CCC(N2CCN(c3ccc(Cl)cc3Cl)C(c3ccc(Cl)cc3)C2)CC1 ; Clc1ccc(C2CN(C3CCC4(CCCC4)CC3)CCN2c2ccc(Cl)cc2Cl)cc1 ; FC(F)(F)C1CCCC(N2CCN(c3ccc(Cl)cc3Cl)C(c3ccc(Cl)cc3)C2)C1]

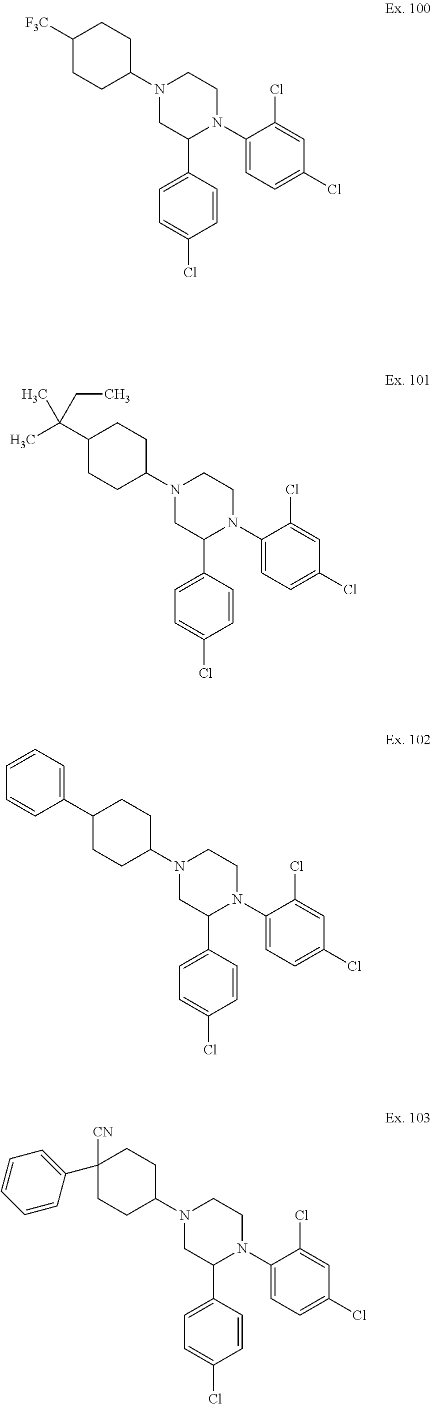 [SMILES: FC(F)(F)C1CCC(N2CCN(c3ccc(Cl)cc3Cl)C(c3ccc(Cl)cc3)C2)CC1 ; CCC(C)(C)C1CCC(N2CCN(c3ccc(Cl)cc3Cl)C(c3ccc(Cl)cc3)C2)CC1 ; Clc1ccc(C2CN(C3CCC(c4ccccc4)CC3)CCN2c2ccc(Cl)cc2Cl)cc1 ; N#CC1(c2ccccc2)CCC(N2CCN(c3ccc(Cl)cc3Cl)C(c3ccc(Cl)cc3)C2)CC1]

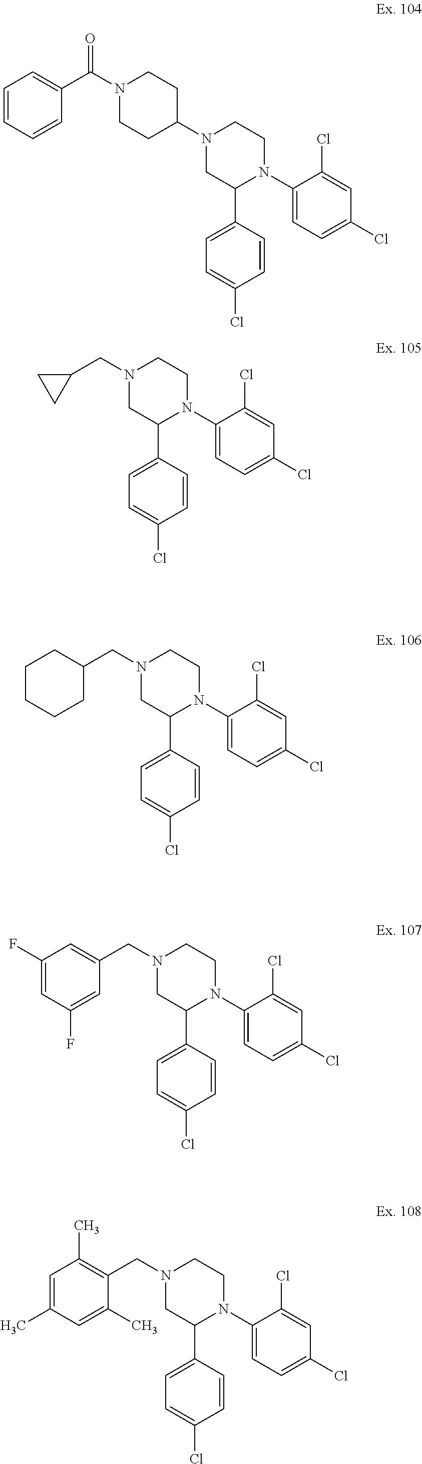 [SMILES: O=C(c1ccccc1)N1CCC(N2CCN(c3ccc(Cl)cc3Cl)C(c3ccc(Cl)cc3)C2)CC1 ; Clc1ccc(C2CN(CC3CC3)CCN2c2ccc(Cl)cc2Cl)cc1 ; Clc1ccc(C2CN(CC3CCCCC3)CCN2c2ccc(Cl)cc2Cl)cc1 ; Fc1cc(F)cc(CN2CCN(c3ccc(Cl)cc3Cl)C(c3ccc(Cl)cc3)C2)c1 ; Cc1cc(C)c(CN2CCN(c3ccc(Cl)cc3Cl)C(c3ccc(Cl)cc3)C2)c(C)c1]

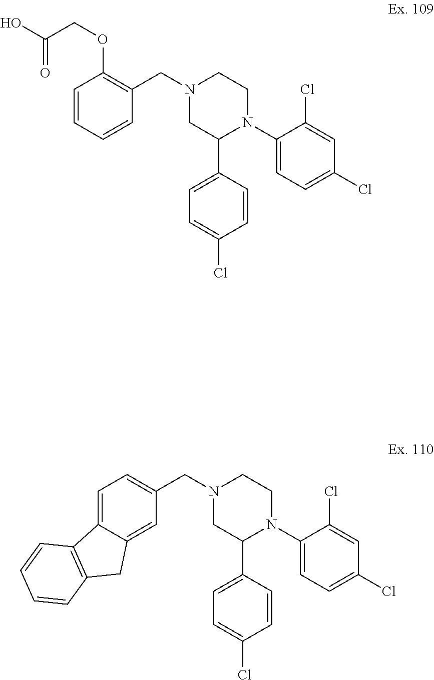 [SMILES: O=C(O)COc1ccccc1CN1CCN(c2ccc(Cl)cc2Cl)C(c2ccc(Cl)cc2)C1 ; Clc1ccc(C2CN(Cc3ccc4c(c3)Cc3ccccc3-4)CCN2c2ccc(Cl)cc2Cl)cc1]

Examples 20-110 were prepared by the following method, using a parallel synthesis approach.

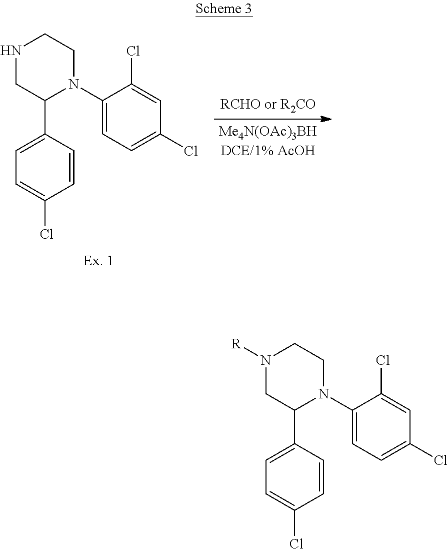

The piperazine Example 1, prepared as described above (0.72 g, 2.1 mmol) and Me₄N(OAc)₃BH (1.11 g, 4.2 mmol) was dissolved in a 1% acetic acid/dichloroethane (DCE) solution (100 mL). Aliquots (1 mL) of the resulting mixture were added to 96 wells of a deep well polypropylene microtiter plate. To each of the wells was then added one of 96 different aldehydes or ketones (shown in Table I, below) (1.0 M solution in MeCN, 150 μL) via a TECAN liquid handler.

TABLE I
Aldehydes/Ketones Used

| Example | Ketone/Aldehyde |
|---|---|
| 20 | 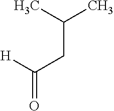 |
| 21 | 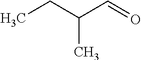 |
| 22 | 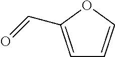 |
| 23 | 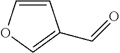 |
| 24 | 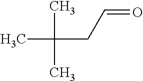 |
| 25 | 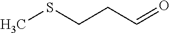 |
| 26 | 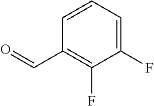 |
| 27 | 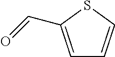 |
| 28 | 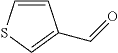 |
| 29 | 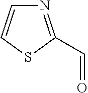 |
| 30 | 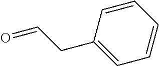 |
| 31 | 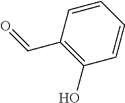 |
| 32 | 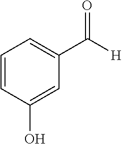 |
| 33 | 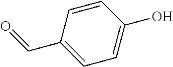 |
| 34 | 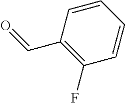 |
| 35 | 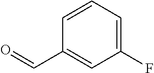 |
| 36 | 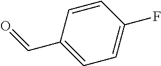 |
| 37 | 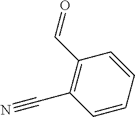 |
| 38 | 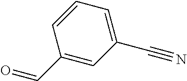 |
| 39 | 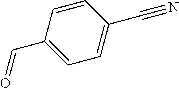 |
| 40 | 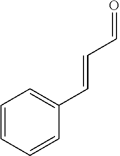 |
| 41 | 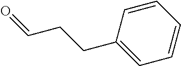 |
| 42 | 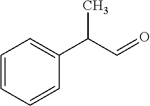 |
| 43 | 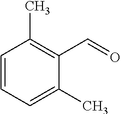 |
| 44 | 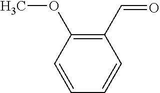 |
| 45 | 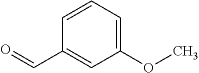 |
| 46 | 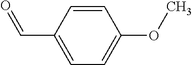 |
| 47 | 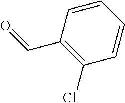 |
| 48 | 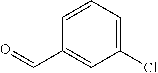 |
| 49 | 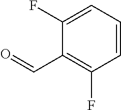 |
| 50 | 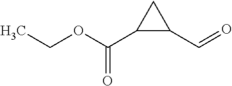 |
| 51 | 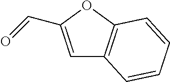 [SMILES: O=Cc1cc2ccccc2o1] |
| 52 | 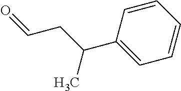 |
| 53 | 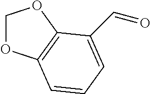 [SMILES: O=Cc1cccc2c1OCO2] |
| 54 | 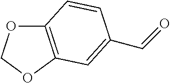 [SMILES: O=Cc1ccc2c(c1)OCO2] |
| 55 | 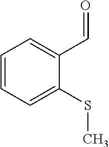 |
| 56 | 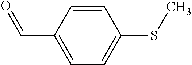 |
| 57 | 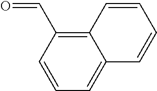 [SMILES: O=Cc1cccc2ccccc12] |
| 58 | 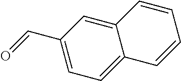 [SMILES: O=Cc1ccc2ccccc2c1] |
| 59 | 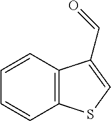 [SMILES: O=Cc1csc2ccccc12] |
| 60 | 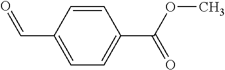 |
| 61 | 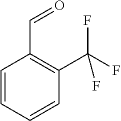 [SMILES: O=Cc1ccccc1C(F)(F)F] |
| 62 | 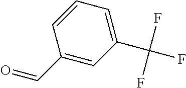 [SMILES: O=Cc1cccc(C(F)(F)F)c1] |
| 63 | 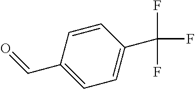 [SMILES: O=Cc1ccc(C(F)(F)F)cc1] |
| 64 | 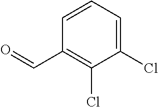 |
| 65 | 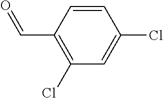 |
| 66 | 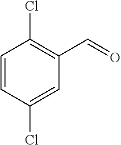 |
| 67 | 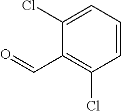 |
| 68 | 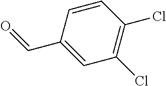 |
| 69 | 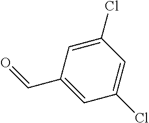 |
| 70 | 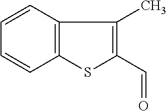 |
| 71 | 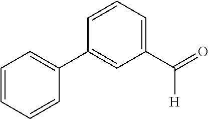 [SMILES: O=Cc1cccc(-c2ccccc2)c1] |
| 72 | 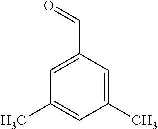 |
| 73 | 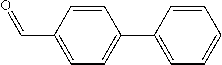 [SMILES: O=Cc1ccc(-c2ccccc2)cc1] |
| 74 | 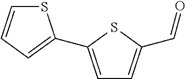 [SMILES: O=Cc1ccc(-c2cccs2)s1] |
| 75 | 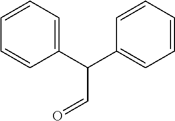 [SMILES: O=CC(c1ccccc1)c1ccccc1] |
| 76 | 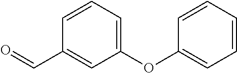 [SMILES: O=Cc1cccc(Oc2ccccc2)c1] |
| 77 | 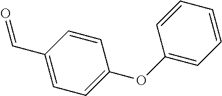 [SMILES: O=Cc1ccc(Oc2ccccc2)cc1] |
| 78 | 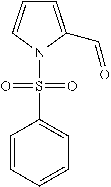 [SMILES: O=Cc1cccn1S(=O)(=O)c1ccccc1] |
| 79 | 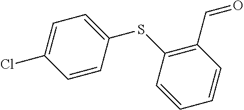 [SMILES: O=Cc1ccccc1Sc1ccc(Cl)cc1] |
| 80 | 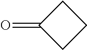 |
| 81 | 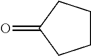 |
| 82 | 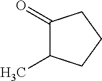 |
| 83 | 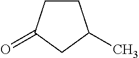 |
| 84 | 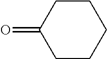 |
| 85 | 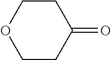 |
| 86 | 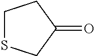 |
| 87 | 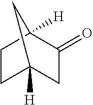 |
| 88 | 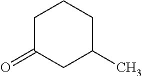 |
| 89 | 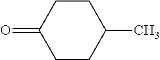 |
| 90 | 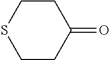 |
| 91 | 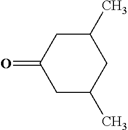 |
| 92 | 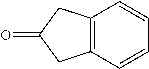 |
| 93 | 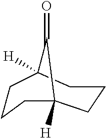 |
| 94 | 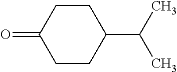 |
| 95 | 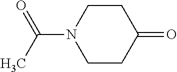 |
| 96 | 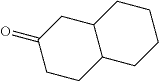 [SMILES: O=C1CCC2CCCCC2C1] |
| 97 | 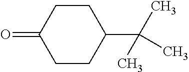 |
| 98 | 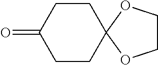 [SMILES: O=C1CCC2(CC1)OCCO2] |
| 99 | 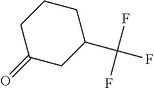 [SMILES: O=C1CCCC(C(F)(F)F)C1] |
| 100 | 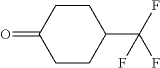 [SMILES: O=C1CCC(C(F)(F)F)CC1] |
| 101 | 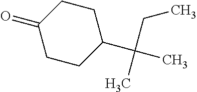 |
| 102 | 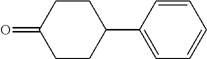 [SMILES: O=C1CCC(c2ccccc2)CC1] |
| 103 | 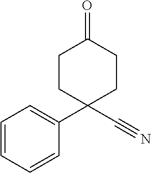 [SMILES: N#CC1(c2ccccc2)CCC(=O)CC1] |
| 104 | 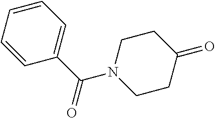 [SMILES: O=C1CCN(C(=O)c2ccccc2)CC1] |
| 105 | 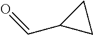 |
| 106 | 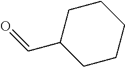 |
| 107 | 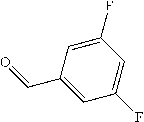 |
| 108 | 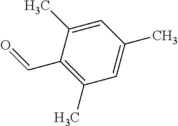 |
| 109 | 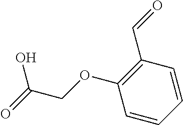 [SMILES: O=Cc1ccccc1OCC(=O)O] |
| 110 | 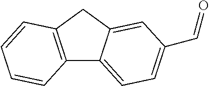 [SMILES: O=Cc1ccc2c(c1)Cc1ccccc1-2] |

The plate was then sealed and shaken at room temperature for 3 days. MP-TsOH resin (i.e., macroporous resin functionalized with toluenesulfonic acid groups; available from Argonaut Technologies, Inc.) (100 mg) was then added to each well of the plate. The plate was then resealed and shaken for 2 h. The bottom of the plate was opened and the filtrate from each well was collected in the corresponding 2 mL well of a 96-well plate. The resin in each well was washed with CH₂Cl₂ (4×) followed by MeOH (3×). The bottom of the plate was then resealed and an aliquot of 2 N NH₃/MeOH (1.5 mL) was added to each well to remove crude product bound to the MP-TsOH resin. The plate was sealed and shaken for 2 h. The bottom of the plate was opened and the filtrate from each well was collected in the corresponding 2 mL well of a 96-well plate. The resin in each well was washed with MeOH (1×). An aliquot from each well was removed for LC/MS analysis. The remaining solution from each well was transferred to 96 corresponding 2-dram bar-coded vials using a TECAN liquid handler. The solvent was then removed from each of the vials using a SPEEDVAC concentrator, to provide crude Products I.

LC/MS analysis showed that most crude Products I contained 10-40% of starting piperazine Example 1. The desired product was also determined to be present in the filtrate by TLC (i.e., thin layer chromatography) analysis. The filtrates were then transferred by a TECAN liquid handler to 96 corresponding BOHDAN MINIBLOCK cartridges containing approximately 100 mg of MP-TsOH resin. The cartridges were sealed and shaken for 20 h. The solvent was then removed from each cartridge in vacuo and the resin was washed in each cartridge with CH₂Cl₂ (3×) and MeOH (3×, 1.5 mL). A 3.5 N NH₃/MeOH-THF (1:1) solution (1.5 mL) was then added to the resin in each cartridge to remove crude product bound to the MP-TsOH resin. The cartridges were sealed and shaken for 20 h. The solvent from each cartridge was collected by filtration and the resin in each cartridge was treated with 7 N NH₃/MeOH (1.5 mL) for 8 h. The solvent was removed by filtration and the filtrates were combined in 96 corresponding 2-dram bar-coded vials. An aliquot form each vial was analyzed by LC/MS, and the remaining solvent from each vial was removed in vacuo to provide crude Products II.

The MP-TsOH resin from Product I was manually transferred with MeOH from the plate to 96 corresponding cartridges of a BOHDAN MINIBLOCK. The solution from each cartridge was then removed by filtration and the resin in each cartridge was treated with 3.5 N NH₃ in MeOH/THF (1:1, 18 h, 1.5 mL). The filtrate from each cartridge was collected and the resin in each cartridge was again treated with 3.5 N NH₃ in MeOH/THF (1:1, 8 h, 1.5 mL). The filtrates were combined with the corresponding Product I and an aliquot from each combined filtrate was submitted for LC/MS analysis. The solvent from each sample was removed in vacuo to provide Product III.

Products II were added to Products III if it was determined by LC/MS analysis that a sufficient quantity of desired product was present in Product II. The combined products were transferred with DCE/MeCN (i.e. dichloroethane/acetonitrile, 1:1, 3 mL) to 96 corresponding BOHDAN MINIBLOCK cartridges containing PS-NCO resin (polystyrene functionalized with isocyanate groups; available from Argonaut Technologies, Inc.) (6 equiv.). The cartridges were capped and shaken for 3 days. The products were filtered into individual vials and the resin was washed with DCE/MeCN (1:1, 2×, 0.5 mL). An aliquot from each vial was removed for LC/MS analysis, and the remaining solvent was removed using a SPEEDVAC concentrator to provide desired products, Examples 20-110.

### Preparation of Examples 111-154 and 171

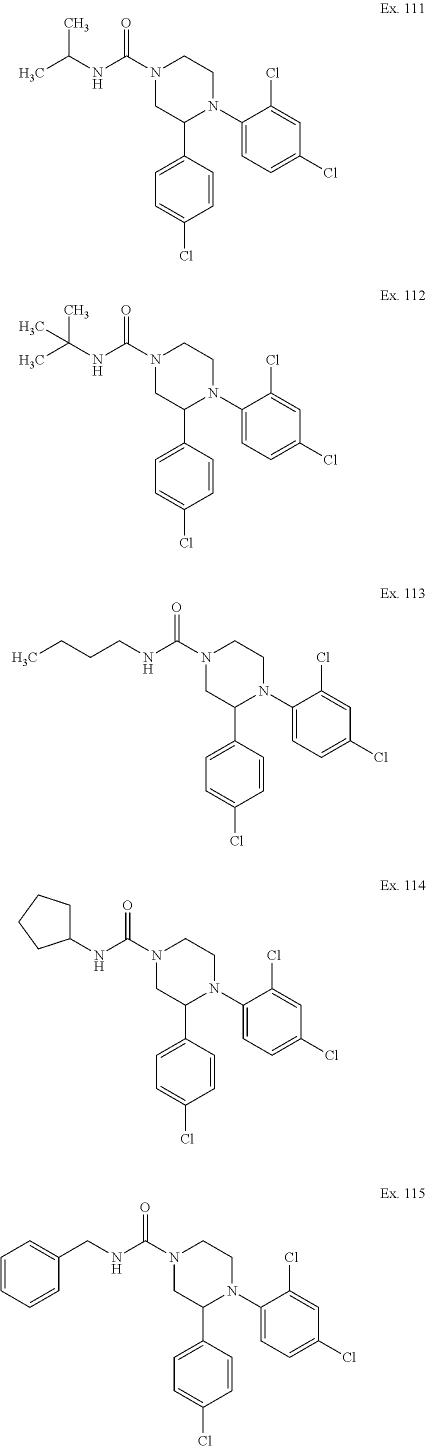 [SMILES: CC(C)NC(=O)N1CCN(c2ccc(Cl)cc2Cl)C(c2ccc(Cl)cc2)C1 ; CC(C)(C)NC(=O)N1CCN(c2ccc(Cl)cc2Cl)C(c2ccc(Cl)cc2)C1 ; CCCCNC(=O)N1CCN(c2ccc(Cl)cc2Cl)C(c2ccc(Cl)cc2)C1 ; O=C(NC1CCCC1)N1CCN(c2ccc(Cl)cc2Cl)C(c2ccc(Cl)cc2)C1 ; O=C(NCc1ccccc1)N1CCN(c2ccc(Cl)cc2Cl)C(c2ccc(Cl)cc2)C1]

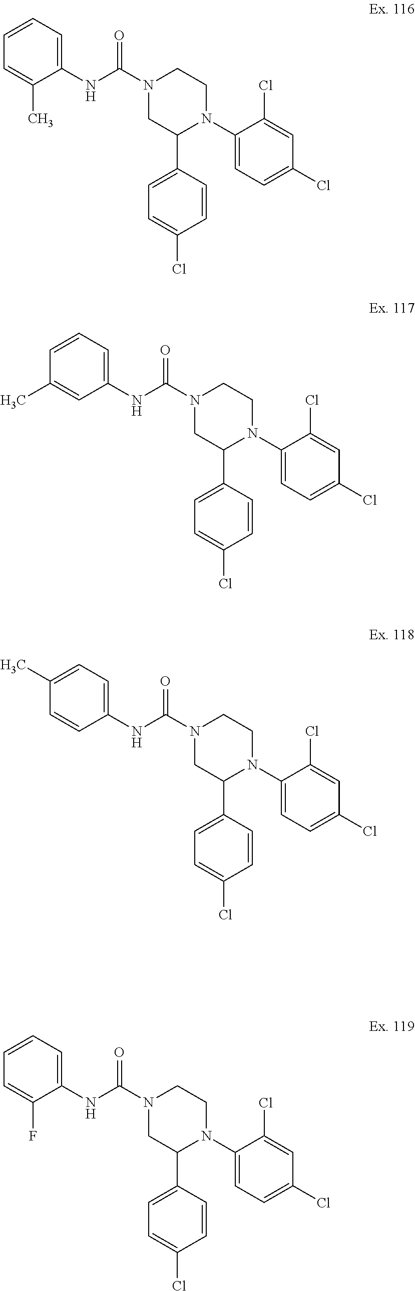 [SMILES: Cc1ccccc1NC(=O)N1CCN(c2ccc(Cl)cc2Cl)C(c2ccc(Cl)cc2)C1 ; Cc1cccc(NC(=O)N2CCN(c3ccc(Cl)cc3Cl)C(c3ccc(Cl)cc3)C2)c1 ; Cc1ccc(NC(=O)N2CCN(c3ccc(Cl)cc3Cl)C(c3ccc(Cl)cc3)C2)cc1 ; O=C(Nc1ccccc1F)N1CCN(c2ccc(Cl)cc2Cl)C(c2ccc(Cl)cc2)C1]

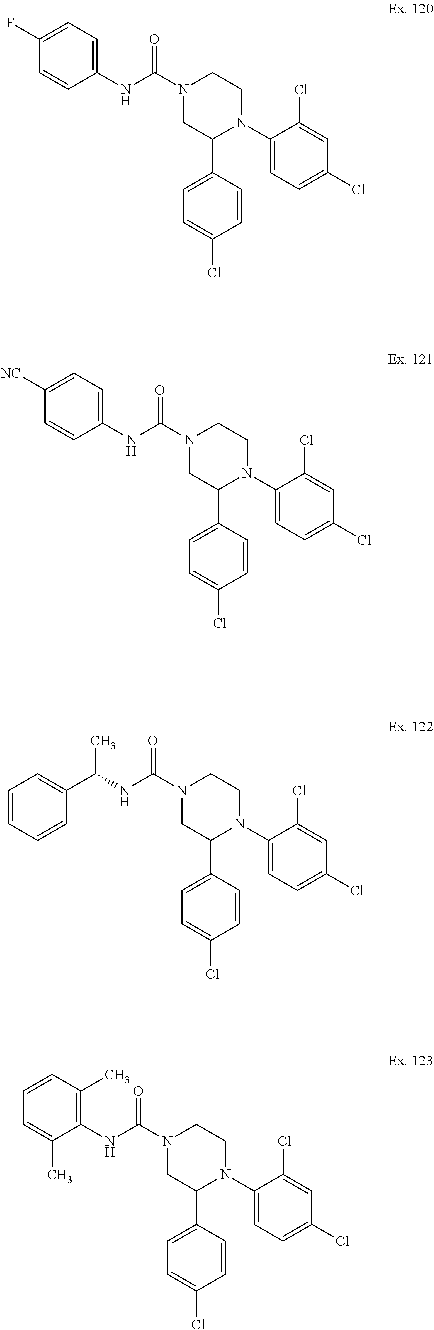 [SMILES: O=C(Nc1ccc(F)cc1)N1CCN(c2ccc(Cl)cc2Cl)C(c2ccc(Cl)cc2)C1 ; N#Cc1ccc(NC(=O)N2CCN(c3ccc(Cl)cc3Cl)C(c3ccc(Cl)cc3)C2)cc1 ; C[C@H](NC(=O)N1CCN(c2ccc(Cl)cc2Cl)C(c2ccc(Cl)cc2)C1)c1ccccc1 ; Cc1cccc(C)c1NC(=O)N1CCN(c2ccc(Cl)cc2Cl)C(c2ccc(Cl)cc2)C1]

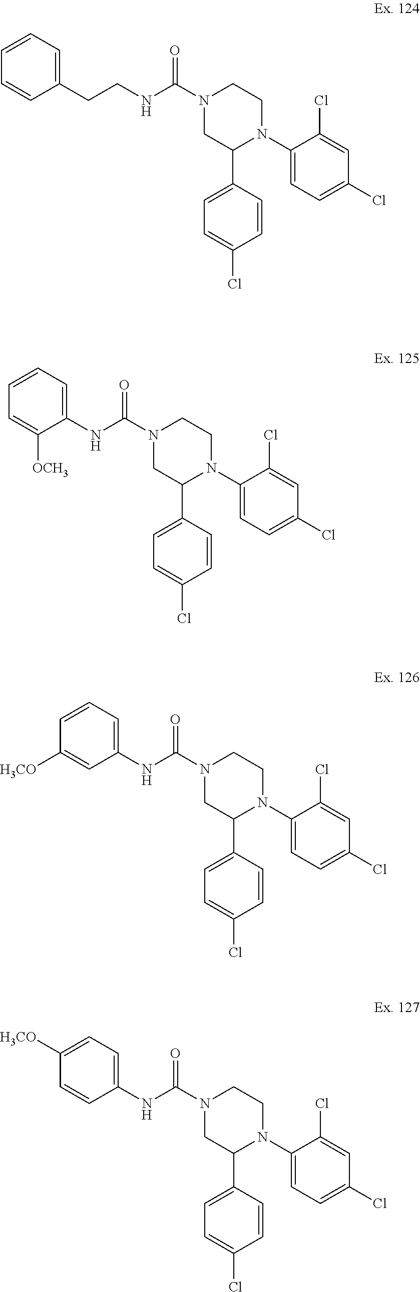 [SMILES: O=C(NCCc1ccccc1)N1CCN(c2ccc(Cl)cc2Cl)C(c2ccc(Cl)cc2)C1 ; COc1ccccc1NC(=O)N1CCN(c2ccc(Cl)cc2Cl)C(c2ccc(Cl)cc2)C1 ; COc1cccc(NC(=O)N2CCN(c3ccc(Cl)cc3Cl)C(c3ccc(Cl)cc3)C2)c1 ; COc1ccc(NC(=O)N2CCN(c3ccc(Cl)cc3Cl)C(c3ccc(Cl)cc3)C2)cc1]

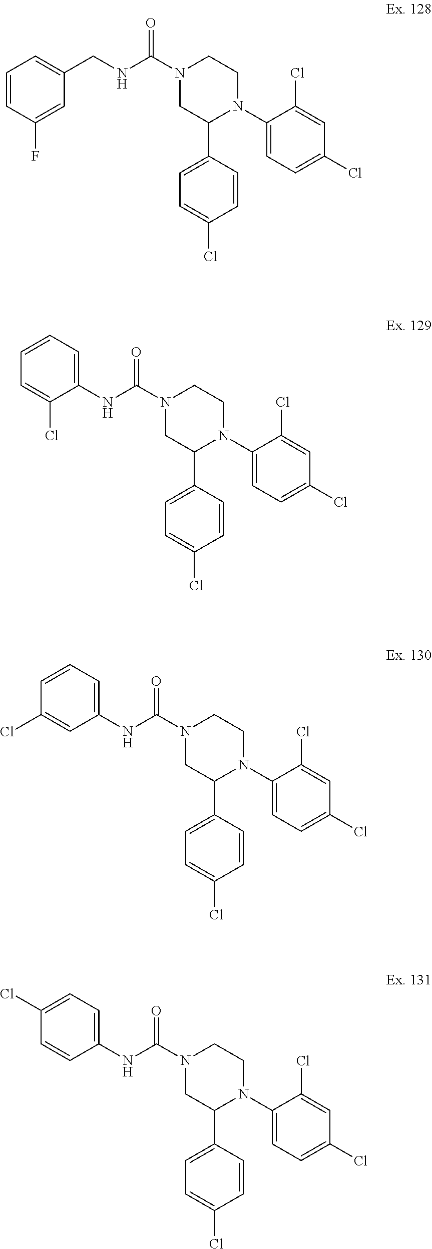 [SMILES: O=C(NCc1cccc(F)c1)N1CCN(c2ccc(Cl)cc2Cl)C(c2ccc(Cl)cc2)C1 ; O=C(Nc1ccccc1Cl)N1CCN(c2ccc(Cl)cc2Cl)C(c2ccc(Cl)cc2)C1 ; O=C(Nc1cccc(Cl)c1)N1CCN(c2ccc(Cl)cc2Cl)C(c2ccc(Cl)cc2)C1 ; O=C(Nc1ccc(Cl)cc1)N1CCN(c2ccc(Cl)cc2Cl)C(c2ccc(Cl)cc2)C1]

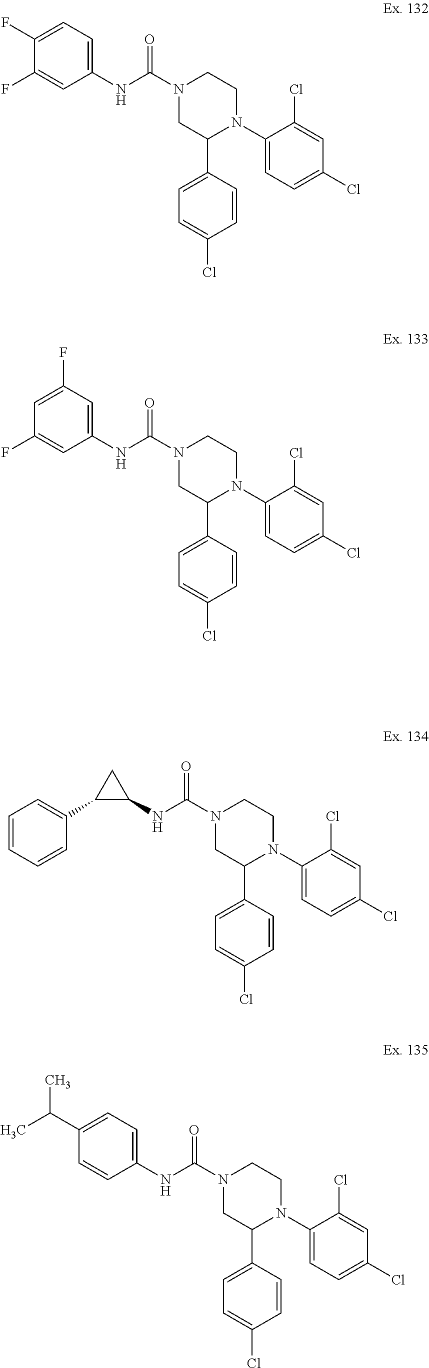 [SMILES: O=C(Nc1ccc(F)c(F)c1)N1CCN(c2ccc(Cl)cc2Cl)C(c2ccc(Cl)cc2)C1 ; O=C(Nc1cc(F)cc(F)c1)N1CCN(c2ccc(Cl)cc2Cl)C(c2ccc(Cl)cc2)C1 ; O=C(N[C@@H]1C[C@H]1c1ccccc1)N1CCN(c2ccc(Cl)cc2Cl)C(c2ccc(Cl)cc2)C1 ; CC(C)c1ccc(NC(=O)N2CCN(c3ccc(Cl)cc3Cl)C(c3ccc(Cl)cc3)C2)cc1]

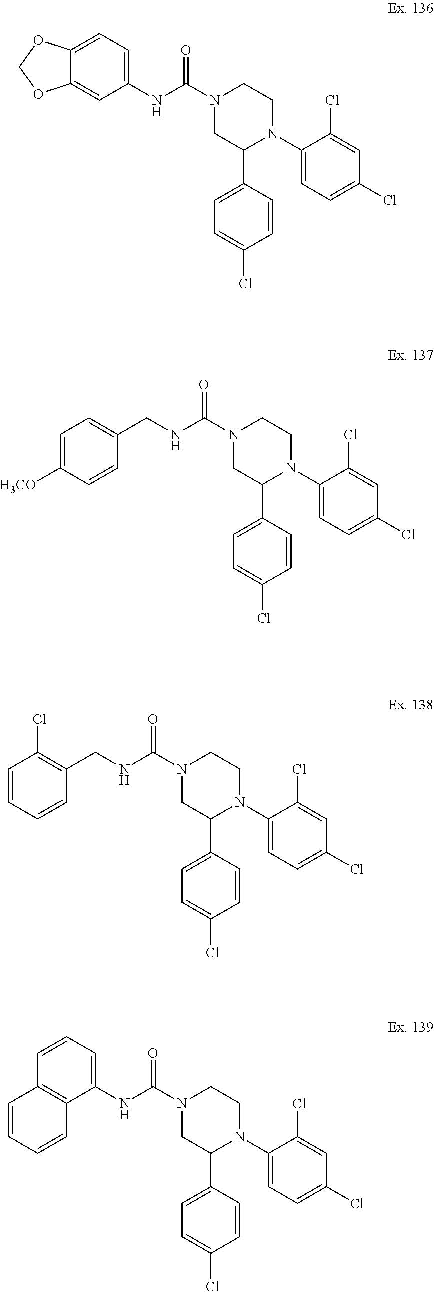 [SMILES: O=C(Nc1ccc2c(c1)OCO2)N1CCN(c2ccc(Cl)cc2Cl)C(c2ccc(Cl)cc2)C1 ; COc1ccc(CNC(=O)N2CCN(c3ccc(Cl)cc3Cl)C(c3ccc(Cl)cc3)C2)cc1 ; O=C(NCc1ccccc1Cl)N1CCN(c2ccc(Cl)cc2Cl)C(c2ccc(Cl)cc2)C1 ; O=C(Nc1cccc2ccccc12)N1CCN(c2ccc(Cl)cc2Cl)C(c2ccc(Cl)cc2)C1]

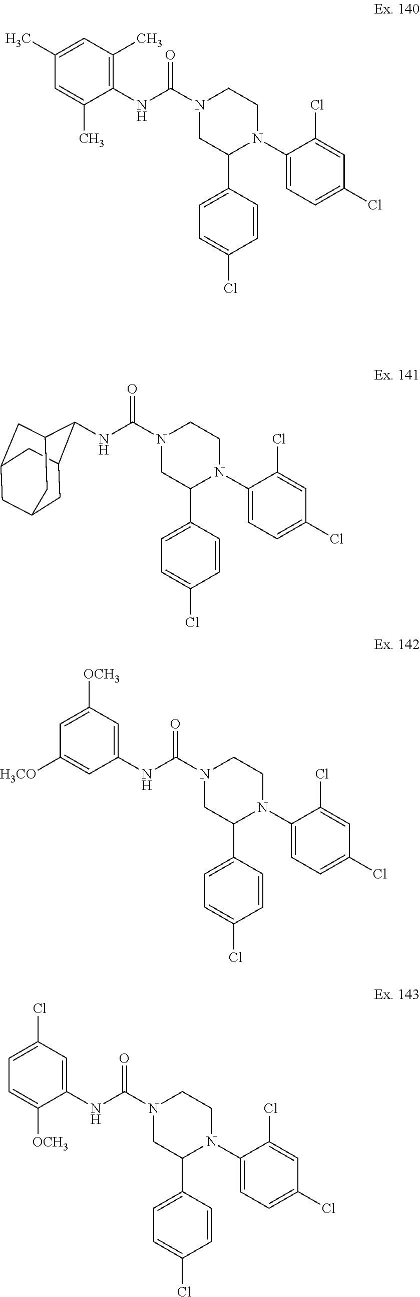 [SMILES: Cc1cc(C)c(NC(=O)N2CCN(c3ccc(Cl)cc3Cl)C(c3ccc(Cl)cc3)C2)c(C)c1 ; O=C(NC1C2CC3CC(C2)CC1C3)N1CCN(c2ccc(Cl)cc2Cl)C(c2ccc(Cl)cc2)C1 ; COc1cc(NC(=O)N2CCN(c3ccc(Cl)cc3Cl)C(c3ccc(Cl)cc3)C2)cc(OC)c1 ; COc1ccc(Cl)cc1NC(=O)N1CCN(c2ccc(Cl)cc2Cl)C(c2ccc(Cl)cc2)C1]

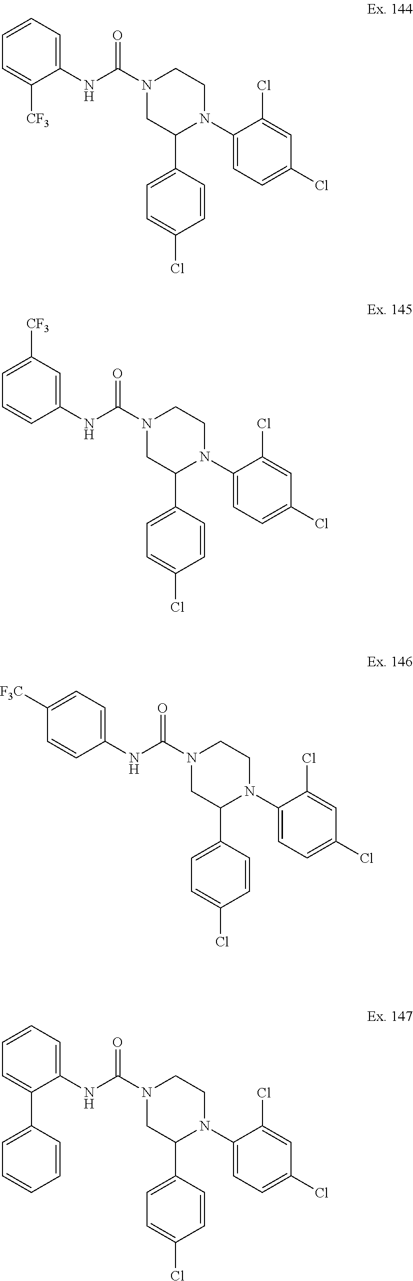 [SMILES: O=C(Nc1ccccc1C(F)(F)F)N1CCN(c2ccc(Cl)cc2Cl)C(c2ccc(Cl)cc2)C1 ; O=C(Nc1cccc(C(F)(F)F)c1)N1CCN(c2ccc(Cl)cc2Cl)C(c2ccc(Cl)cc2)C1 ; O=C(Nc1ccc(C(F)(F)F)cc1)N1CCN(c2ccc(Cl)cc2Cl)C(c2ccc(Cl)cc2)C1 ; O=C(Nc1ccccc1-c1ccccc1)N1CCN(c2ccc(Cl)cc2Cl)C(c2ccc(Cl)cc2)C1]

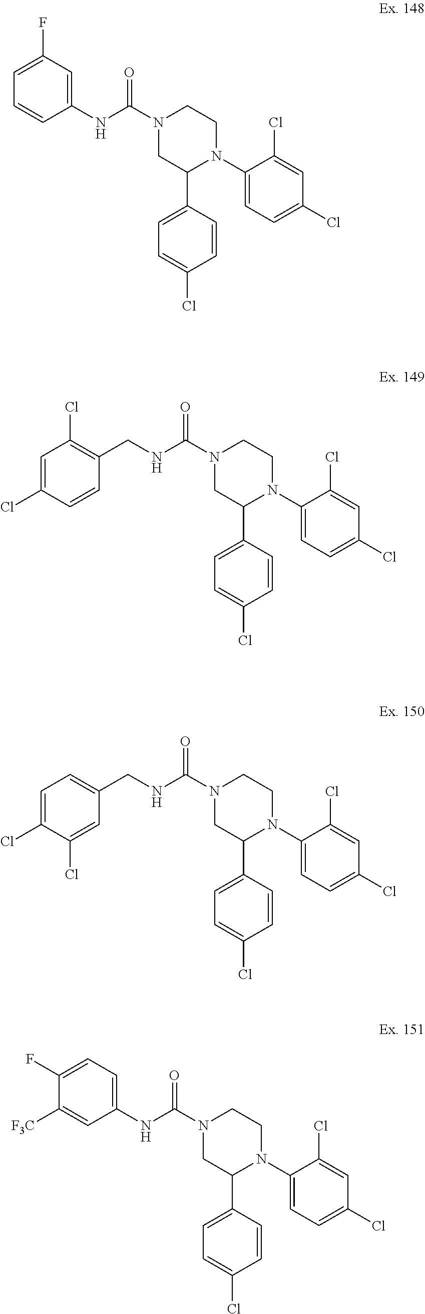 [SMILES: O=C(Nc1cccc(F)c1)N1CCN(c2ccc(Cl)cc2Cl)C(c2ccc(Cl)cc2)C1 ; O=C(NCc1ccc(Cl)cc1Cl)N1CCN(c2ccc(Cl)cc2Cl)C(c2ccc(Cl)cc2)C1 ; O=C(NCc1ccc(Cl)c(Cl)c1)N1CCN(c2ccc(Cl)cc2Cl)C(c2ccc(Cl)cc2)C1 ; O=C(Nc1ccc(F)c(C(F)(F)F)c1)N1CCN(c2ccc(Cl)cc2Cl)C(c2ccc(Cl)cc2)C1]

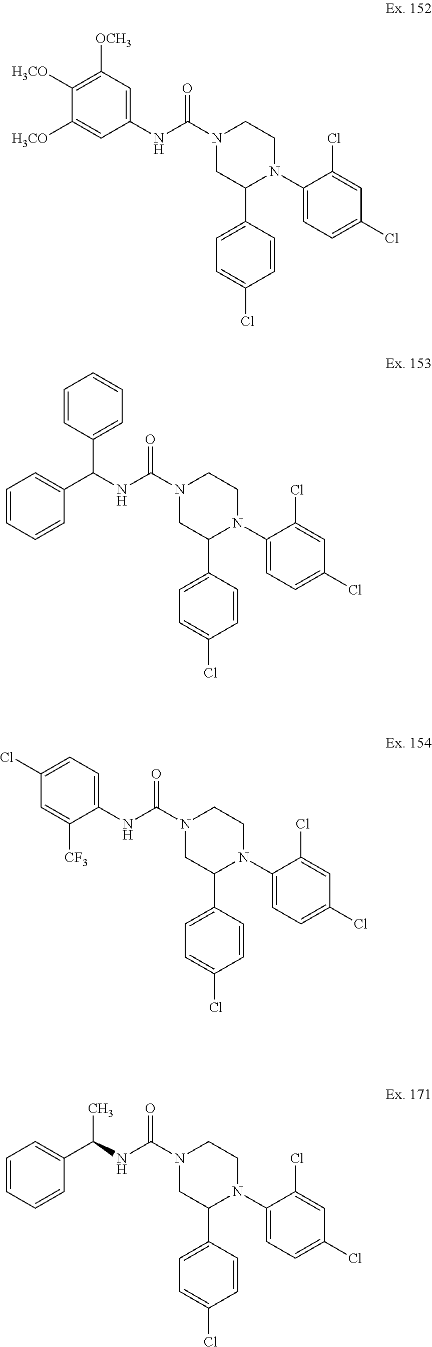 [SMILES: COc1cc(NC(=O)N2CCN(c3ccc(Cl)cc3Cl)C(c3ccc(Cl)cc3)C2)cc(OC)c1OC ; O=C(NC(c1ccccc1)c1ccccc1)N1CCN(c2ccc(Cl)cc2Cl)C(c2ccc(Cl)cc2)C1 ; O=C(Nc1ccc(Cl)cc1C(F)(F)F)N1CCN(c2ccc(Cl)cc2Cl)C(c2ccc(Cl)cc2)C1 ; C[C@@H](NC(=O)N1CCN(c2ccc(Cl)cc2Cl)C(c2ccc(Cl)cc2)C1)c1ccccc1]

Examples 111-154 and 171 were prepared using the following parallel synthetic method.

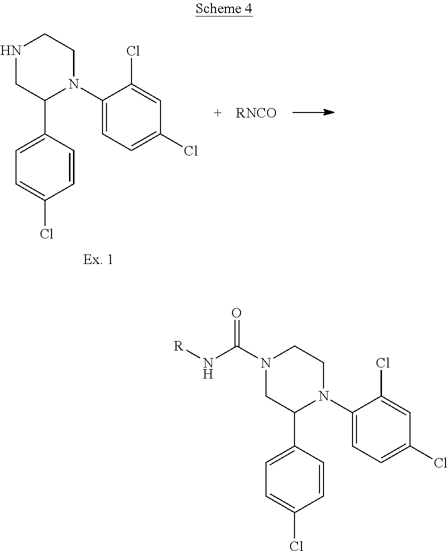

A dichloroethane:acetonitrile (1:1) stock solution of piperazine Example 1 (1 mL, 0.023 mmol) was added to 48 wells of a deep well polypropylene microtiter plate. 0.5 M stock solutions of each individual isocyanates (RNCO) (Table II, below) in dichloromethane (0.14 mL, 0.07 mmol) were added to the plate, which was then sealed and shaken at 25° C. for 20 h. The solutions were then filtered through a polypropylene frit into a second microtiter plate containing PS-Isocyanate resin (i.e., polystyrene resin functionalized with isocyanate groups, available from Argonaut Technologies, Inc.) (0.046 g, 0.07 mmol) and PS-Trisamine resin (i.e., polystyrene resin functionalized with the tris-(2-aminoethyl)amine group, available from Argonaut Technologies, Inc.) (0.042 g, 0.18 mmol). After the top plate was washed with MeCN (acetonitrile) (0.5 mL/well), the plate was removed, the bottom plate sealed, and then shaken for 16 h at 25° C. The solutions were then filtered through a polypropylene frit into a 96-well collection plate. The wells of the top plate were washed with MeCN (0.5 mL/well) and the top plate removed. The resultant solutions in the collection plate were transferred into vials and the solvent removed in vacuo using a SPEEDVAC. The resulting samples were evaluated by LC/MS and those that were >70% pure are listed above.

TABLE II
Isocyanates Used

| Example | Isocyanate |
|---|---|
| 111 | 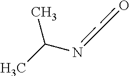 |
| 112 | 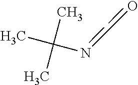 |
| 113 | 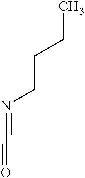 |
| 114 | 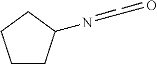 |
| 115 | 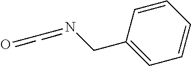 |
| 116 | 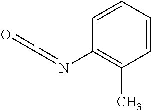 |
| 117 | 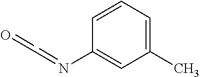 |
| 118 | 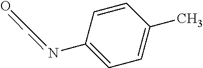 |
| 119 | 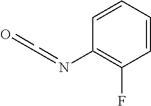 |
| 120 | 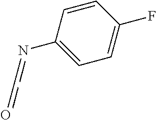 |
| 121 | 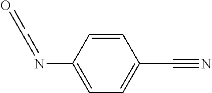 [SMILES: N#Cc1ccc(NCO)cc1] |
| 122 | 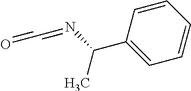 |
| 123 | 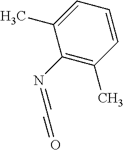 |
| 124 | 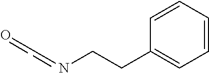 [SMILES: OCNCCc1ccccc1] |
| 125 | 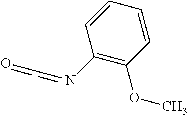 |
| 126 | 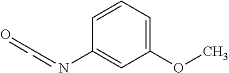 |
| 127 | 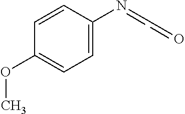 |
| 128 | 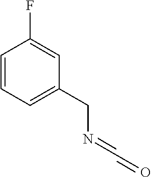 [SMILES: OCNCc1cccc(F)c1] |
| 129 | 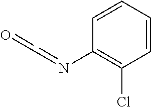 |
| 130 | 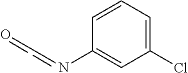 |
| 131 | 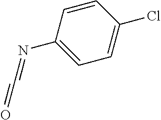 |
| 132 | 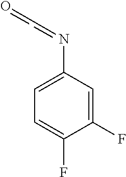 [SMILES: OCNc1ccc(F)c(F)c1] |
| 133 | 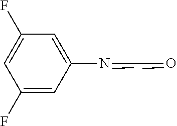 [SMILES: OCNc1cc(F)cc(F)c1] |
| 134 | 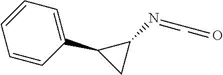 [SMILES: OCN[C@@H]1C[C@H]1c1ccccc1] |
| 135 | 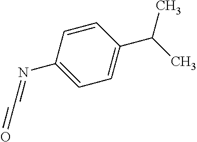 |
| 136 | 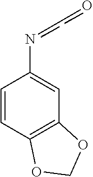 [SMILES: OCNc1ccc2c(c1)OCO2] |
| 137 | 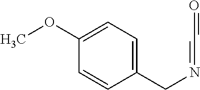 |
| 138 | 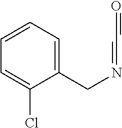 [SMILES: OCNCc1ccccc1Cl] |
| 139 | 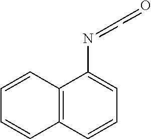 [SMILES: OCNc1cccc2ccccc12] |
| 140 | 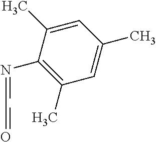 |
| 141 | 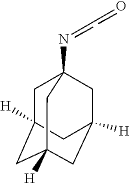 [SMILES: OCN[C@]12C[C@H]3C[C@H](C[C@H](C3)C1)C2] |
| 142 | 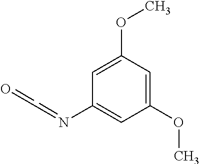 |
| 143 | 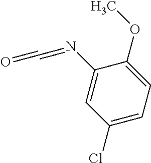 |
| 144 | 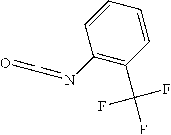 [SMILES: OCNc1ccccc1C(F)(F)F] |
| 145 | 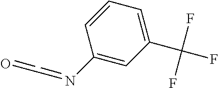 [SMILES: OCNc1cccc(C(F)(F)F)c1] |
| 146 | 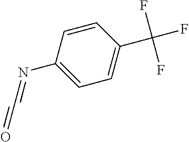 [SMILES: OCNc1ccc(C(F)(F)F)cc1] |
| 147 | 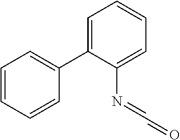 [SMILES: OCNc1ccccc1-c1ccccc1] |
| 148 | 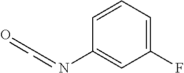 |
| 149 | 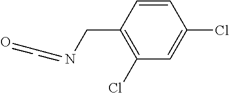 [SMILES: OCNCc1ccc(Cl)cc1Cl] |
| 150 | 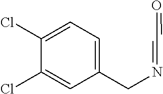 [SMILES: OCNCc1ccc(Cl)c(Cl)c1] |
| 151 | 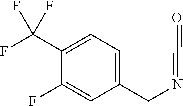 [SMILES: OCNCc1ccc(C(F)(F)F)c(F)c1] |
| 152 | 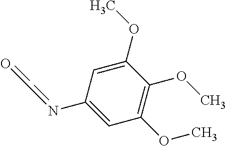 |
| 153 | 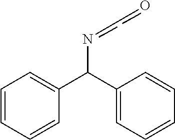 [SMILES: OCNC(c1ccccc1)c1ccccc1] |
| 154 | 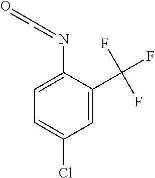 [SMILES: OCNc1ccc(Cl)cc1C(F)(F)F] |
| 171 | 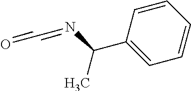 |

### Preparation of Examples 155-170 and 172-233

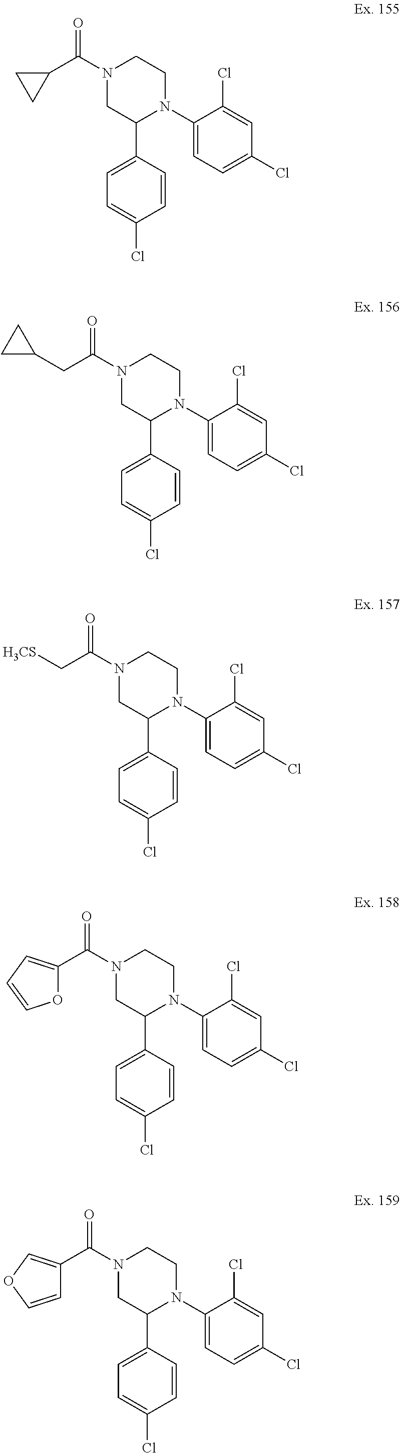

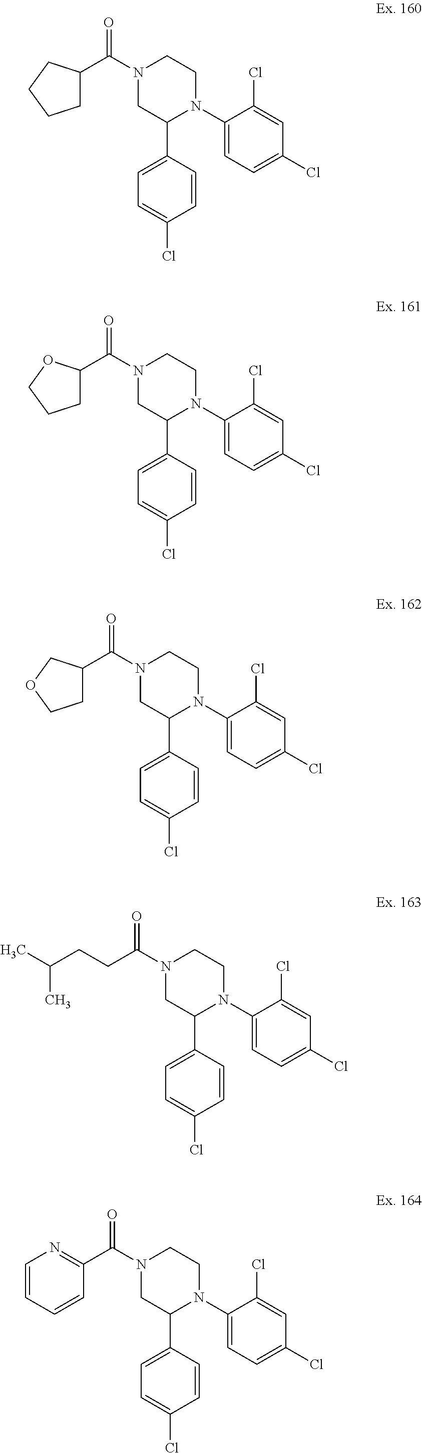 [SMILES: O=C(C1CCCC1)N1CCN(c2ccc(Cl)cc2Cl)C(c2ccc(Cl)cc2)C1 ; O=C(C1CCCO1)N1CCN(c2ccc(Cl)cc2Cl)C(c2ccc(Cl)cc2)C1 ; O=C(C1CCOC1)N1CCN(c2ccc(Cl)cc2Cl)C(c2ccc(Cl)cc2)C1 ; CC(C)CCC(=O)N1CCN(c2ccc(Cl)cc2Cl)C(c2ccc(Cl)cc2)C1 ; O=C(c1ccccn1)N1CCN(c2ccc(Cl)cc2Cl)C(c2ccc(Cl)cc2)C1]

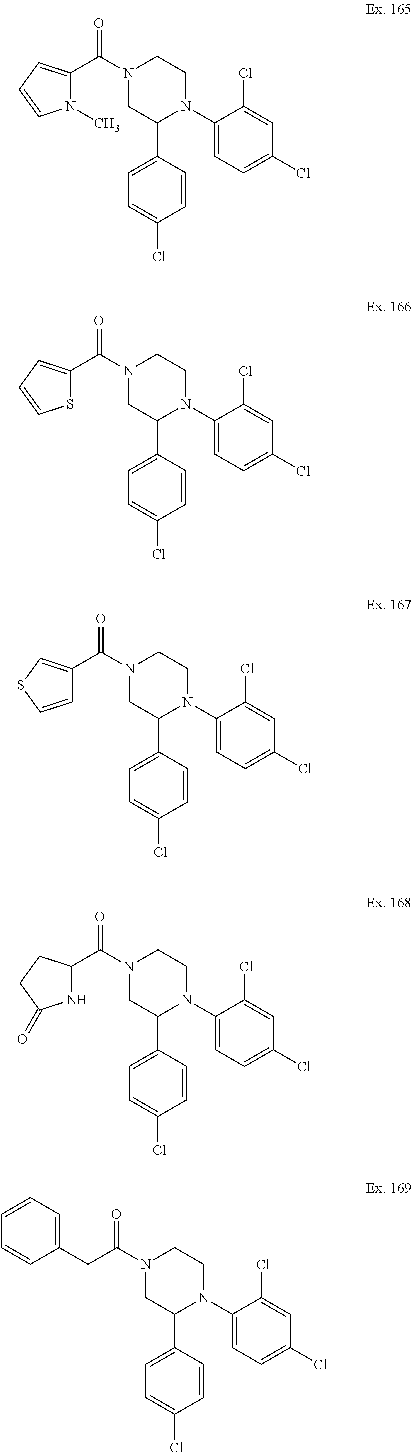 [SMILES: Cn1cccc1C(=O)N1CCN(c2ccc(Cl)cc2Cl)C(c2ccc(Cl)cc2)C1 ; O=C(c1cccs1)N1CCN(c2ccc(Cl)cc2Cl)C(c2ccc(Cl)cc2)C1 ; O=C(c1ccsc1)N1CCN(c2ccc(Cl)cc2Cl)C(c2ccc(Cl)cc2)C1 ; O=C1CCC(C(=O)N2CCN(c3ccc(Cl)cc3Cl)C(c3ccc(Cl)cc3)C2)N1 ; O=C(Cc1ccccc1)N1CCN(c2ccc(Cl)cc2Cl)C(c2ccc(Cl)cc2)C1]

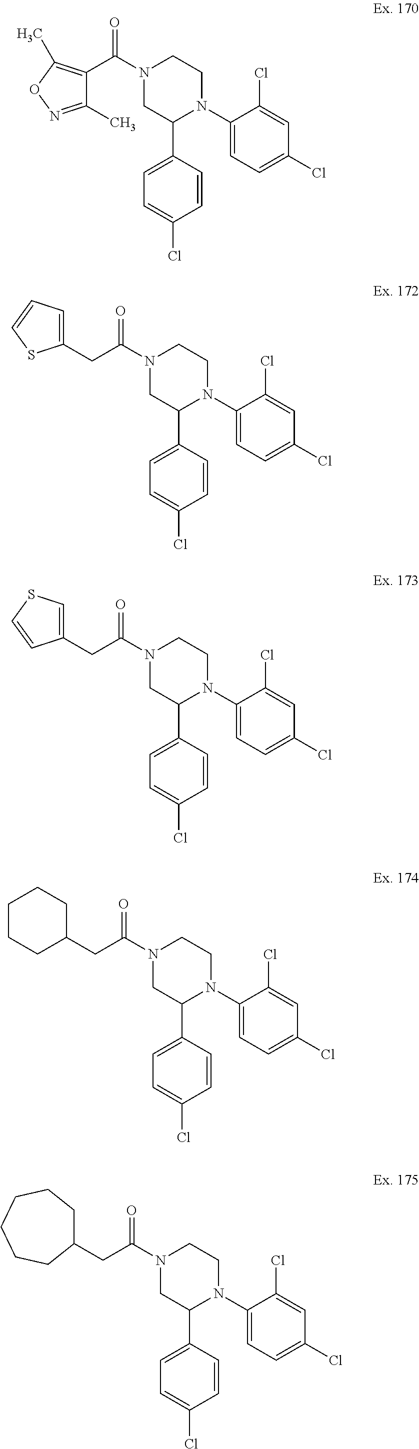 [SMILES: Cc1noc(C)c1C(=O)N1CCN(c2ccc(Cl)cc2Cl)C(c2ccc(Cl)cc2)C1 ; O=C(Cc1cccs1)N1CCN(c2ccc(Cl)cc2Cl)C(c2ccc(Cl)cc2)C1 ; O=C(Cc1ccsc1)N1CCN(c2ccc(Cl)cc2Cl)C(c2ccc(Cl)cc2)C1 ; O=C(CC1CCCCC1)N1CCN(c2ccc(Cl)cc2Cl)C(c2ccc(Cl)cc2)C1 ; O=C(CC1CCCCCC1)N1CCN(c2ccc(Cl)cc2Cl)C(c2ccc(Cl)cc2)C1]

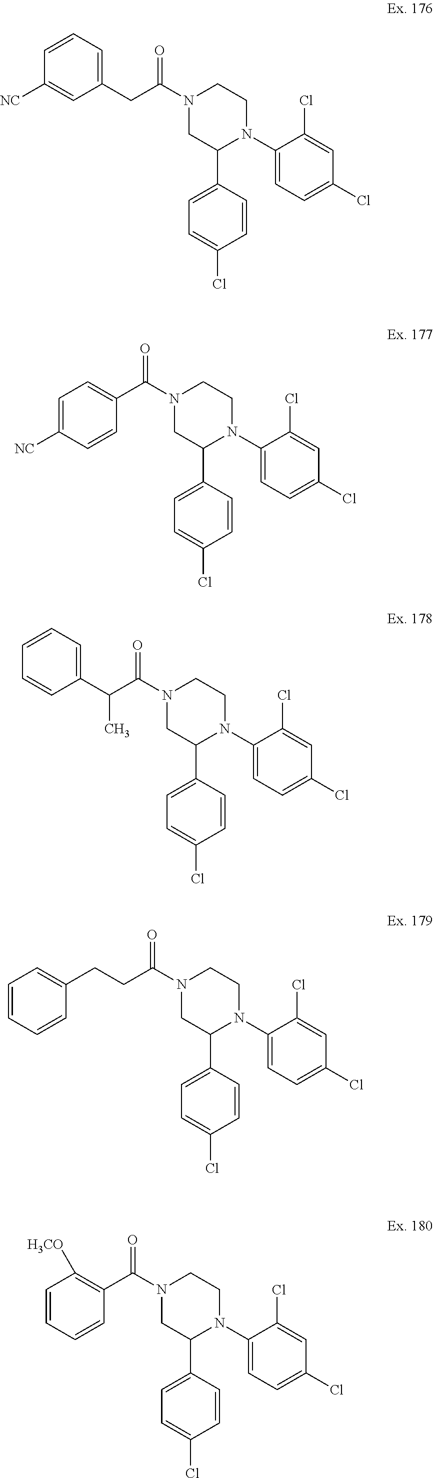 [SMILES: N#Cc1cccc(CC(=O)N2CCN(c3ccc(Cl)cc3Cl)C(c3ccc(Cl)cc3)C2)c1 ; N#Cc1ccc(C(=O)N2CCN(c3ccc(Cl)cc3Cl)C(c3ccc(Cl)cc3)C2)cc1 ; CC(C(=O)N1CCN(c2ccc(Cl)cc2Cl)C(c2ccc(Cl)cc2)C1)c1ccccc1 ; O=C(CCc1ccccc1)N1CCN(c2ccc(Cl)cc2Cl)C(c2ccc(Cl)cc2)C1 ; COc1ccccc1C(=O)N1CCN(c2ccc(Cl)cc2Cl)C(c2ccc(Cl)cc2)C1]

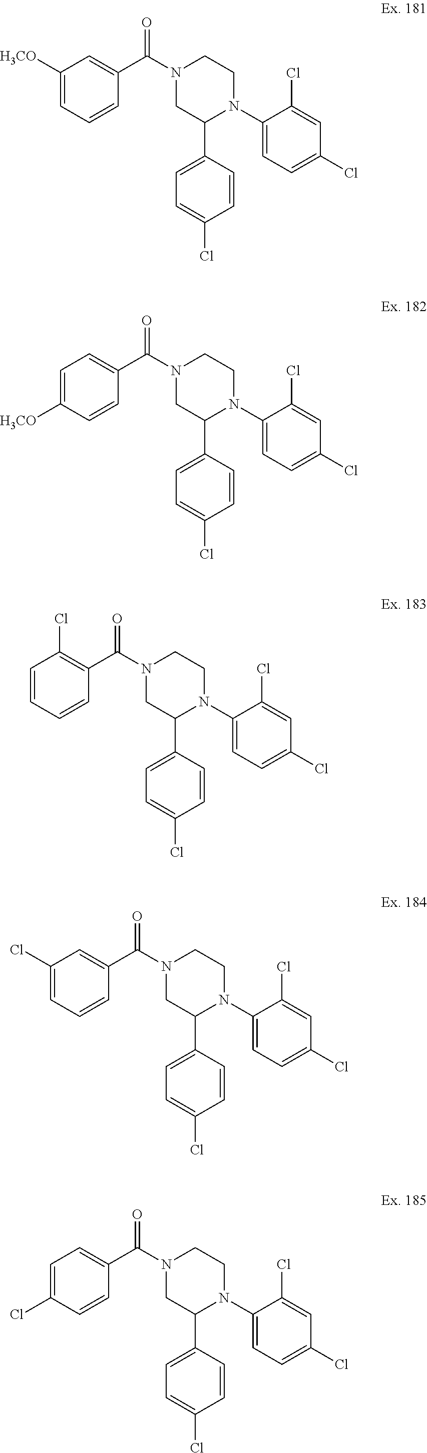 [SMILES: COc1cccc(C(=O)N2CCN(c3ccc(Cl)cc3Cl)C(c3ccc(Cl)cc3)C2)c1 ; COc1ccc(C(=O)N2CCN(c3ccc(Cl)cc3Cl)C(c3ccc(Cl)cc3)C2)cc1 ; O=C(c1ccccc1Cl)N1CCN(c2ccc(Cl)cc2Cl)C(c2ccc(Cl)cc2)C1 ; O=C(c1cccc(Cl)c1)N1CCN(c2ccc(Cl)cc2Cl)C(c2ccc(Cl)cc2)C1 ; O=C(c1ccc(Cl)cc1)N1CCN(c2ccc(Cl)cc2Cl)C(c2ccc(Cl)cc2)C1]

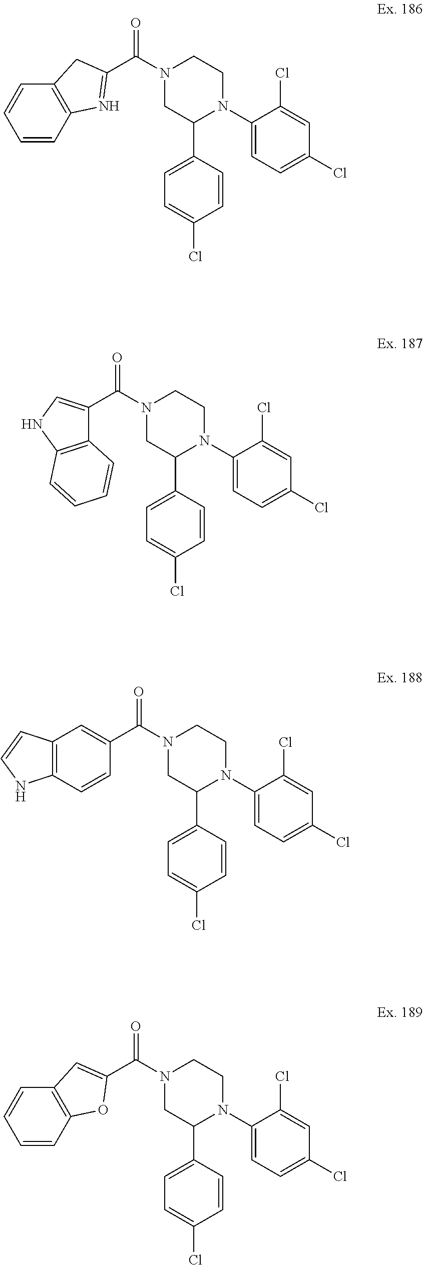 [SMILES: O=C(C1=Nc2ccccc2C1)N1CCN(c2ccc(Cl)cc2Cl)C(c2ccc(Cl)cc2)C1 ; O=C(c1c[nH]c2ccccc12)N1CCN(c2ccc(Cl)cc2Cl)C(c2ccc(Cl)cc2)C1 ; O=C(c1ccc2[nH]ccc2c1)N1CCN(c2ccc(Cl)cc2Cl)C(c2ccc(Cl)cc2)C1 ; O=C(c1cc2ccccc2o1)N1CCN(c2ccc(Cl)cc2Cl)C(c2ccc(Cl)cc2)C1]

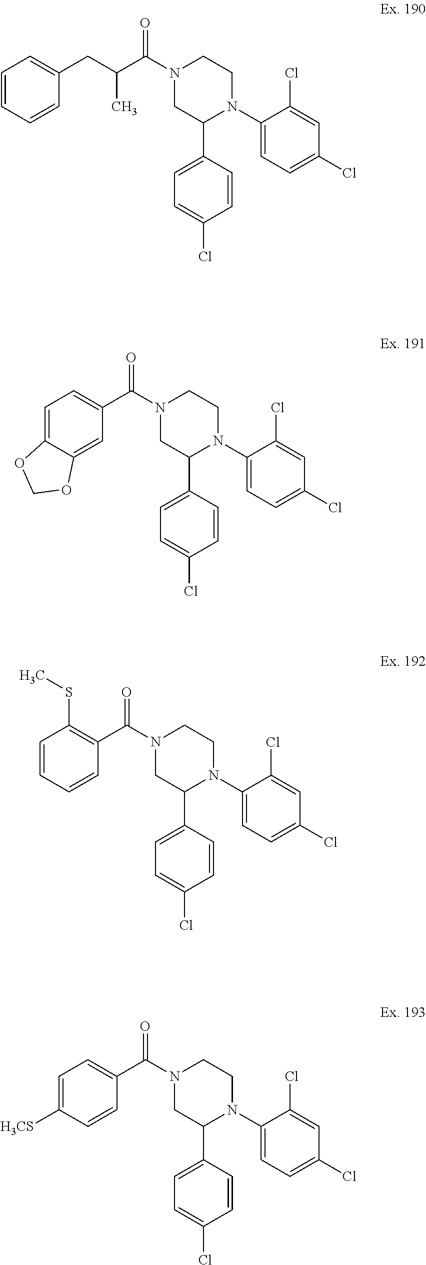

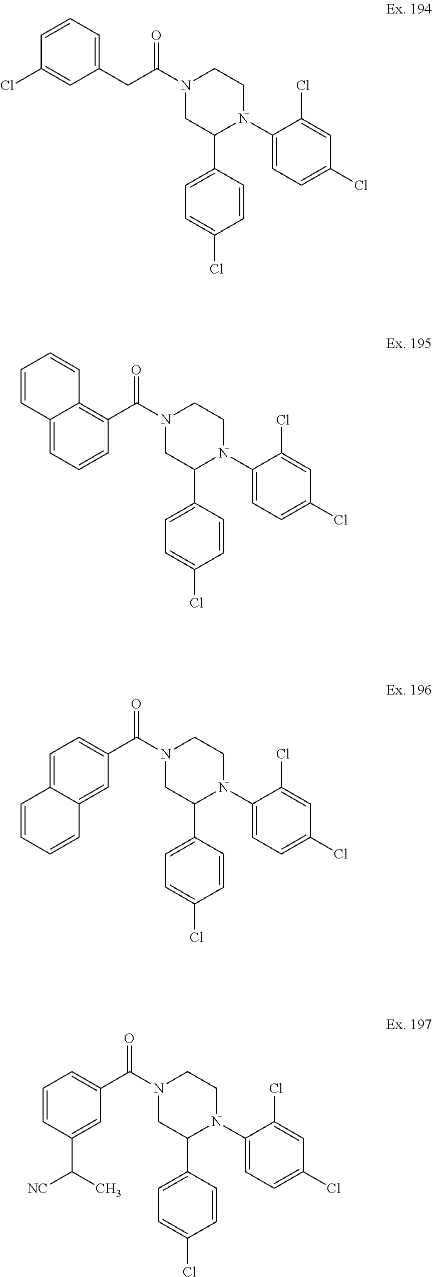 [SMILES: O=C(Cc1cccc(Cl)c1)N1CCN(c2ccc(Cl)cc2Cl)C(c2ccc(Cl)cc2)C1 ; O=C(c1cccc2ccccc12)N1CCN(c2ccc(Cl)cc2Cl)C(c2ccc(Cl)cc2)C1 ; O=C(c1ccc2ccccc2c1)N1CCN(c2ccc(Cl)cc2Cl)C(c2ccc(Cl)cc2)C1 ; CC(C#N)c1cccc(C(=O)N2CCN(c3ccc(Cl)cc3Cl)C(c3ccc(Cl)cc3)C2)c1]

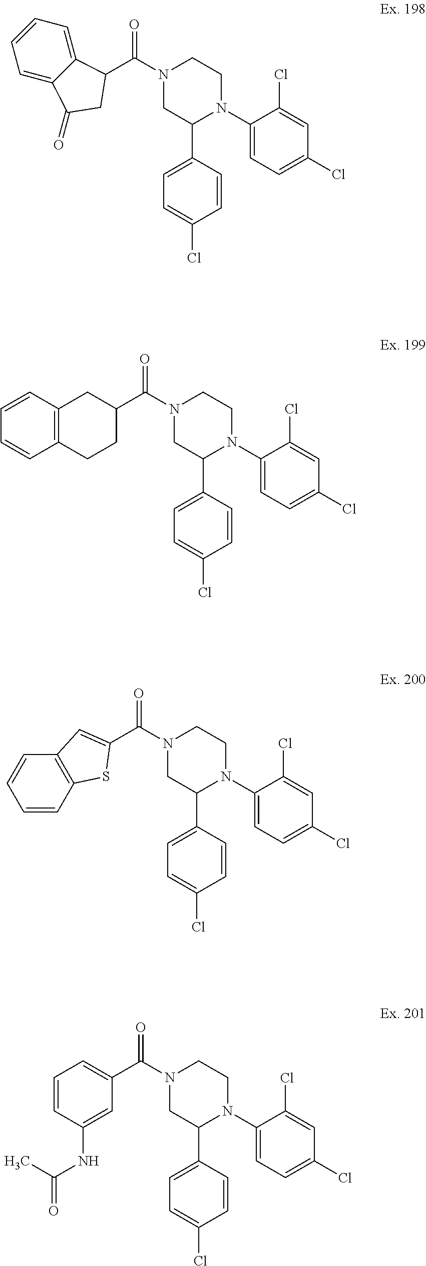 [SMILES: O=C1CC(C(=O)N2CCN(c3ccc(Cl)cc3Cl)C(c3ccc(Cl)cc3)C2)c2ccccc21 ; O=C(C1CCc2ccccc2C1)N1CCN(c2ccc(Cl)cc2Cl)C(c2ccc(Cl)cc2)C1 ; O=C(c1cc2ccccc2s1)N1CCN(c2ccc(Cl)cc2Cl)C(c2ccc(Cl)cc2)C1 ; CC(=O)Nc1cccc(C(=O)N2CCN(c3ccc(Cl)cc3Cl)C(c3ccc(Cl)cc3)C2)c1]

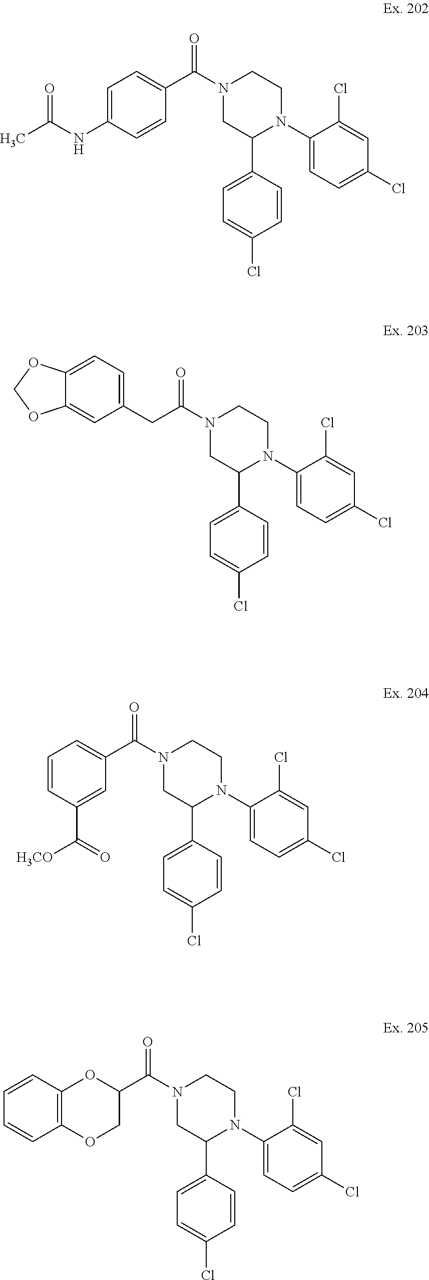 [SMILES: CC(=O)Nc1ccc(C(=O)N2CCN(c3ccc(Cl)cc3Cl)C(c3ccc(Cl)cc3)C2)cc1 ; O=C(Cc1ccc2c(c1)OCO2)N1CCN(c2ccc(Cl)cc2Cl)C(c2ccc(Cl)cc2)C1 ; COC(=O)c1cccc(C(=O)N2CCN(c3ccc(Cl)cc3Cl)C(c3ccc(Cl)cc3)C2)c1 ; O=C(C1COc2ccccc2O1)N1CCN(c2ccc(Cl)cc2Cl)C(c2ccc(Cl)cc2)C1]

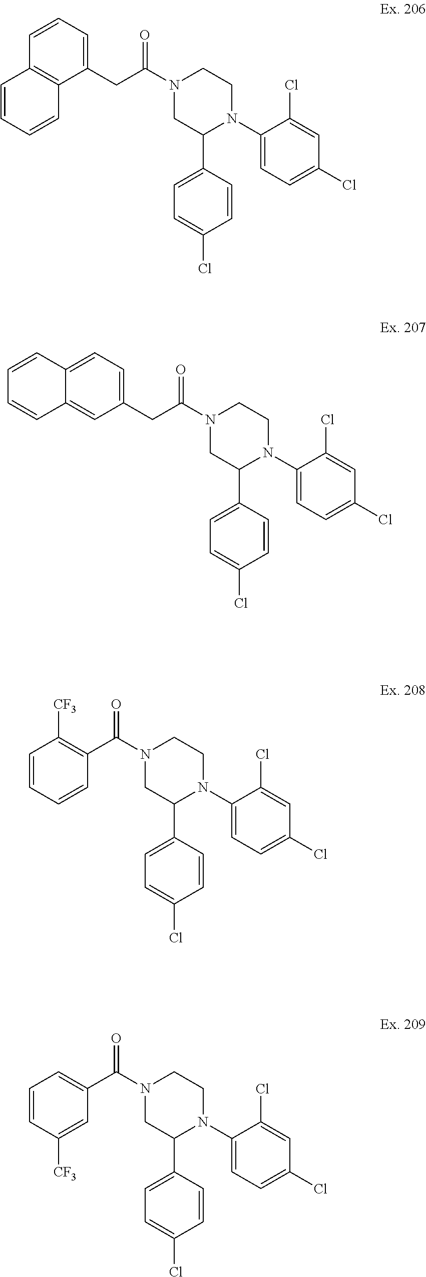 [SMILES: O=C(Cc1cccc2ccccc12)N1CCN(c2ccc(Cl)cc2Cl)C(c2ccc(Cl)cc2)C1 ; O=C(Cc1ccc2ccccc2c1)N1CCN(c2ccc(Cl)cc2Cl)C(c2ccc(Cl)cc2)C1 ; O=C(c1ccccc1C(F)(F)F)N1CCN(c2ccc(Cl)cc2Cl)C(c2ccc(Cl)cc2)C1 ; O=C(c1cccc(C(F)(F)F)c1)N1CCN(c2ccc(Cl)cc2Cl)C(c2ccc(Cl)cc2)C1]

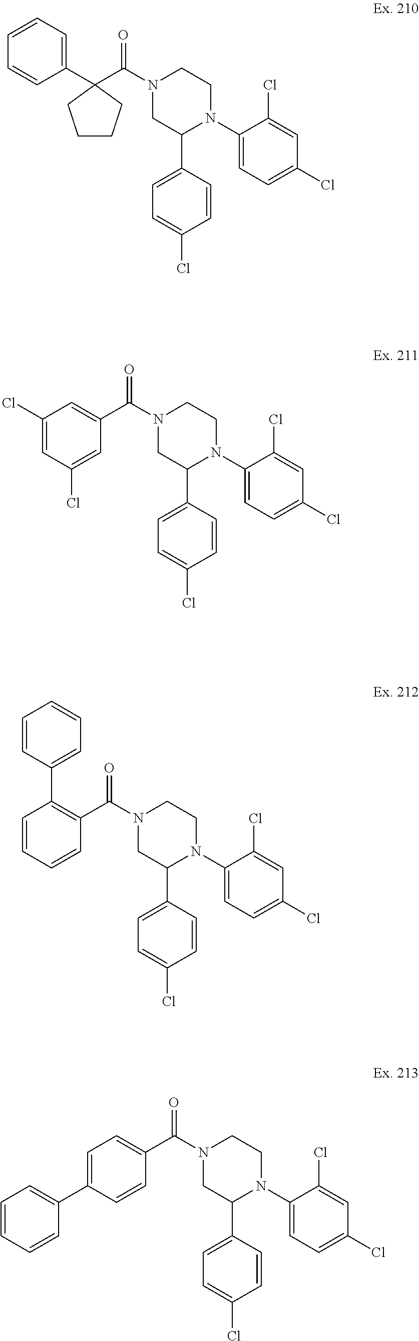 [SMILES: O=C(N1CCN(c2ccc(Cl)cc2Cl)C(c2ccc(Cl)cc2)C1)C1(c2ccccc2)CCCC1 ; O=C(c1cc(Cl)cc(Cl)c1)N1CCN(c2ccc(Cl)cc2Cl)C(c2ccc(Cl)cc2)C1 ; O=C(c1ccccc1-c1ccccc1)N1CCN(c2ccc(Cl)cc2Cl)C(c2ccc(Cl)cc2)C1 ; O=C(c1ccc(-c2ccccc2)cc1)N1CCN(c2ccc(Cl)cc2Cl)C(c2ccc(Cl)cc2)C1]

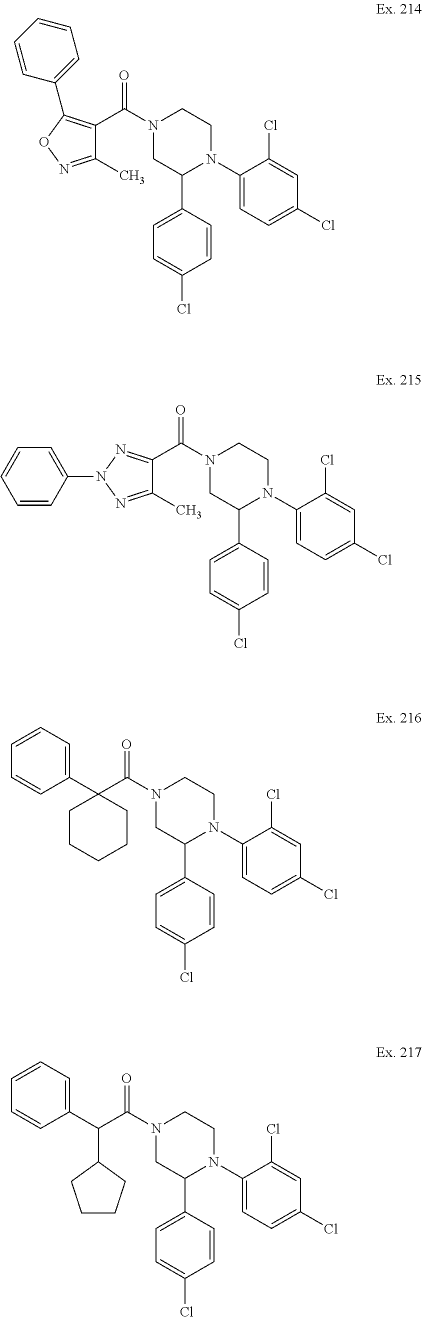 [SMILES: Cc1noc(-c2ccccc2)c1C(=O)N1CCN(c2ccc(Cl)cc2Cl)C(c2ccc(Cl)cc2)C1 ; Cc1nn(-c2ccccc2)nc1C(=O)N1CCN(c2ccc(Cl)cc2Cl)C(c2ccc(Cl)cc2)C1 ; O=C(N1CCN(c2ccc(Cl)cc2Cl)C(c2ccc(Cl)cc2)C1)C1(c2ccccc2)CCCCC1 ; O=C(C(c1ccccc1)C1CCCC1)N1CCN(c2ccc(Cl)cc2Cl)C(c2ccc(Cl)cc2)C1]

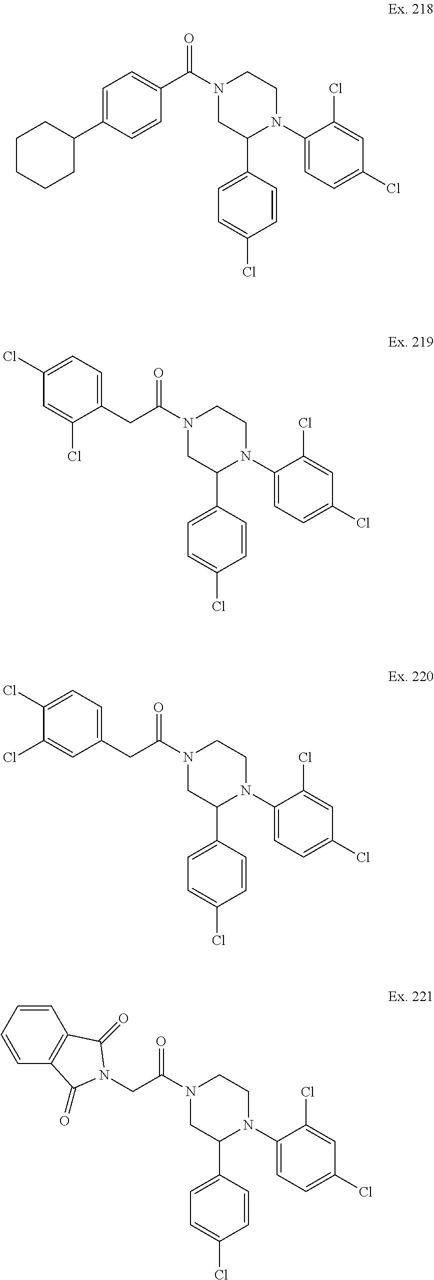 [SMILES: O=C(c1ccc(C2CCCCC2)cc1)N1CCN(c2ccc(Cl)cc2Cl)C(c2ccc(Cl)cc2)C1 ; O=C(Cc1ccc(Cl)cc1Cl)N1CCN(c2ccc(Cl)cc2Cl)C(c2ccc(Cl)cc2)C1 ; O=C(Cc1ccc(Cl)c(Cl)c1)N1CCN(c2ccc(Cl)cc2Cl)C(c2ccc(Cl)cc2)C1 ; O=C(CN1C(=O)c2ccccc2C1=O)N1CCN(c2ccc(Cl)cc2Cl)C(c2ccc(Cl)cc2)C1]

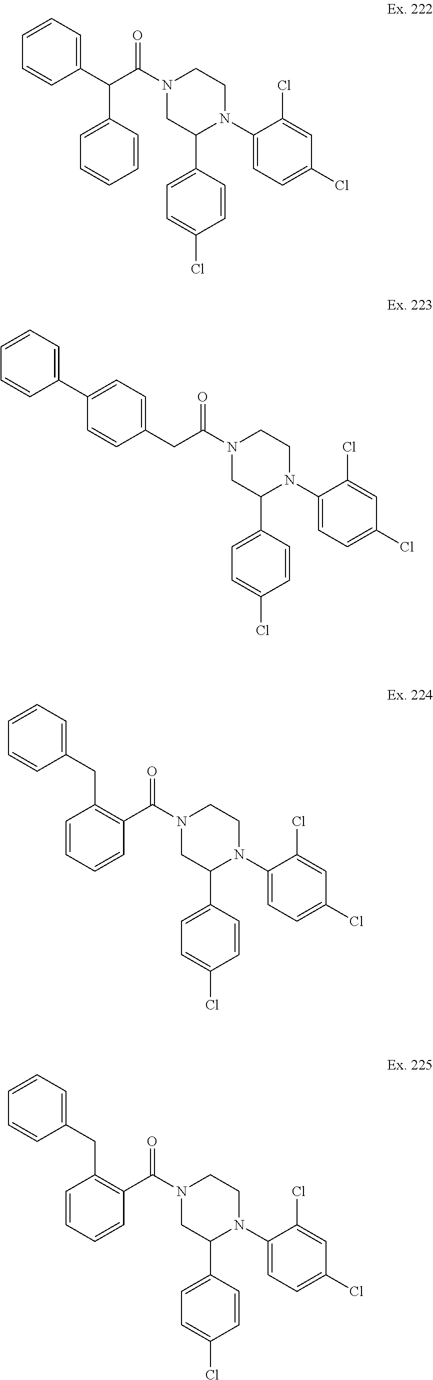 [SMILES: O=C(C(c1ccccc1)c1ccccc1)N1CCN(c2ccc(Cl)cc2Cl)C(c2ccc(Cl)cc2)C1 ; O=C(Cc1ccc(-c2ccccc2)cc1)N1CCN(c2ccc(Cl)cc2Cl)C(c2ccc(Cl)cc2)C1 ; O=C(c1ccccc1Cc1ccccc1)N1CCN(c2ccc(Cl)cc2Cl)C(c2ccc(Cl)cc2)C1 ; O=C(c1ccccc1Cc1ccccc1)N1CCN(c2ccc(Cl)cc2Cl)C(c2ccc(Cl)cc2)C1]

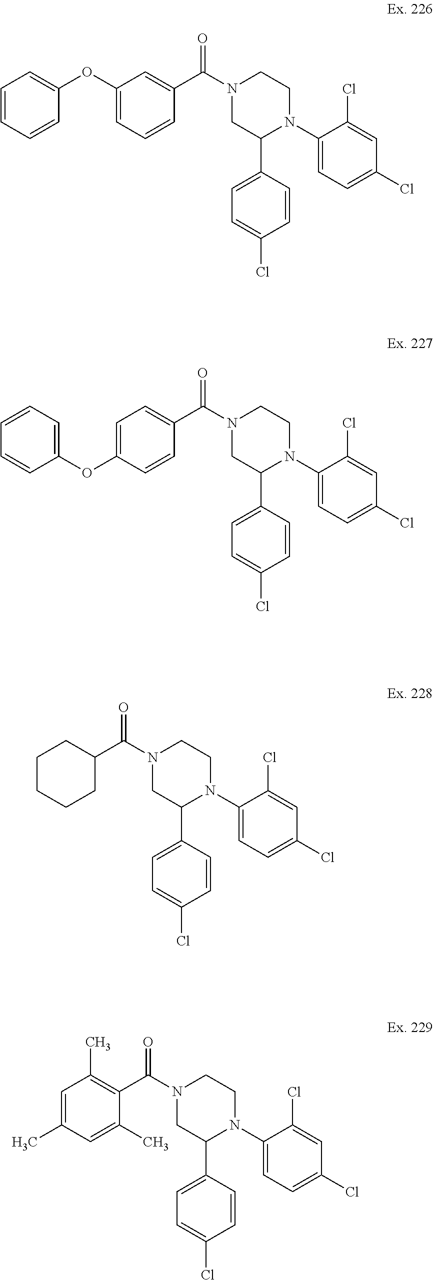 [SMILES: O=C(c1cccc(Oc2ccccc2)c1)N1CCN(c2ccc(Cl)cc2Cl)C(c2ccc(Cl)cc2)C1 ; O=C(c1ccc(Oc2ccccc2)cc1)N1CCN(c2ccc(Cl)cc2Cl)C(c2ccc(Cl)cc2)C1 ; O=C(C1CCCCC1)N1CCN(c2ccc(Cl)cc2Cl)C(c2ccc(Cl)cc2)C1 ; Cc1cc(C)c(C(=O)N2CCN(c3ccc(Cl)cc3Cl)C(c3ccc(Cl)cc3)C2)c(C)c1]

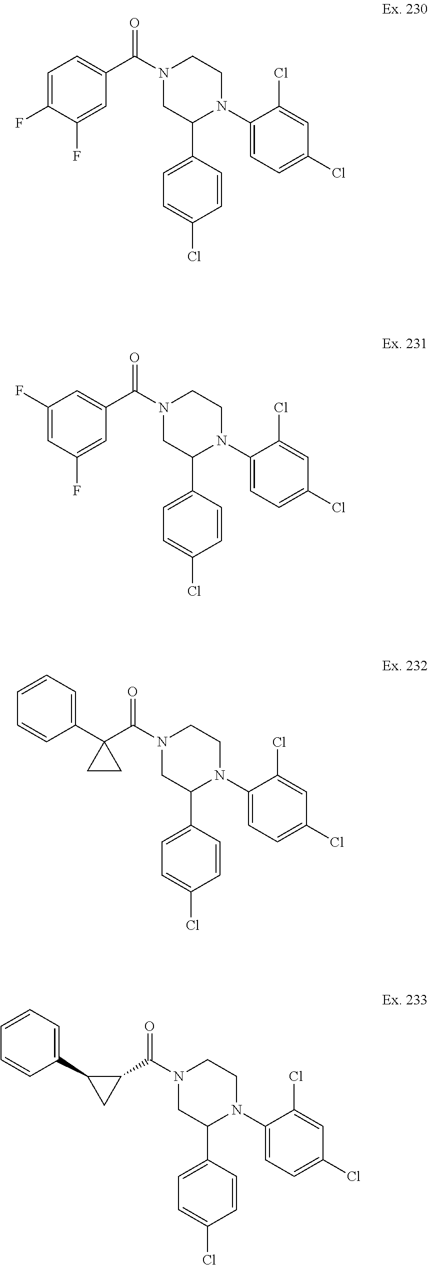 [SMILES: O=C(c1ccc(F)c(F)c1)N1CCN(c2ccc(Cl)cc2Cl)C(c2ccc(Cl)cc2)C1 ; O=C(c1cc(F)cc(F)c1)N1CCN(c2ccc(Cl)cc2Cl)C(c2ccc(Cl)cc2)C1 ; O=C(N1CCN(c2ccc(Cl)cc2Cl)C(c2ccc(Cl)cc2)C1)C1(c2ccccc2)CC1 ; O=C([C@@H]1C[C@H]1c1ccccc1)N1CCN(c2ccc(Cl)cc2Cl)C(c2ccc(Cl)cc2)C1]

Examples 155-170 and 172-233 were prepared using the following parallel synthetic method.

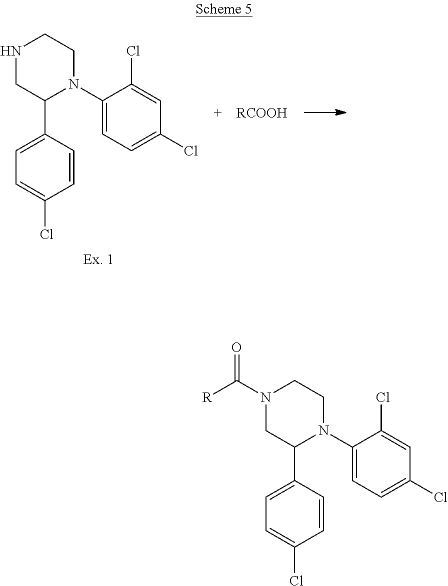

PS-EDC resin (i.e., polystyrene functionalized with EDC-1-(dimethylaminopropyl)-3-ethylcarbodiimide-available from Polymer Laboratories) (0.082 g, 1.42 mmol) was added to 96 wells of a deep well polypropylene microtiter plate followed by a MeCN/THF (3:2) stock solution (1 mL) of piperazine Example 1 (0.021 mmol) and HOBt (i.e., 1-hydroxybenzotriazole hydrate) (0.031 mmol). 1 M stock solutions of each of the individual acids (RCOOH) (Table III, below) (0.042 mL, 0.042 mmol) were added to the wells, which were then sealed and shaken at 25° C. for 18 h. The solutions were filtered through a polypropylene frit into a second microtiter plate containing PS-Isocyanate resin (3 equiv., 0.07 mmol) and PS-Trisamine resin (8 equiv., 0.17 mmol). After the top plate was washed with MeCN (0.5 mL/well), the plate was removed, the bottom microtiter plate was sealed and then shaken at 25° C. for 16 h. The solutions were filtered through a polypropylene frit into a 96-well collection plate. The wells of the top plate were then washed with MeCN (0.5 mL/well), and the plate removed. The resultant solutions in the collection plate were transferred into vials and the solvent removed in vacuo using a SPEEDVAC. The resulting samples were evaluated by LCMS and those that were >70% pure are shown above.

TABLE III
Carboxylic Acids Used to Prepare Examples 155-170 and 172-233

| Example | Carboxylic Acid Structure |
|---|---|
| 155 | 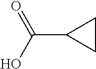 |
| 156 | 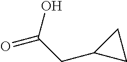 |
| 157 | 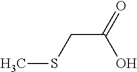 |
| 158 | 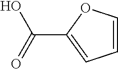 |
| 159 | 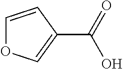 |
| 160 | 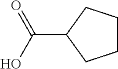 |
| 161 | 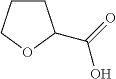 |
| 162 | 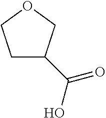 |
| 163 | 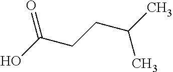 |
| 164 | 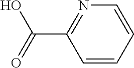 |
| 165 | 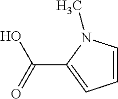 |
| 166 | 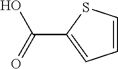 |
| 167 | 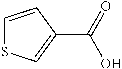 |
| 168 | 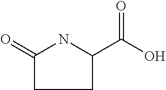 |
| 169 | 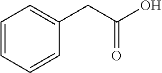 |
| 170 | 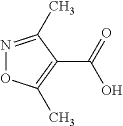 |
| 172 | 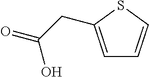 |
| 173 | 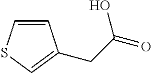 |
| 174 | 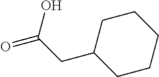 |
| 175 | 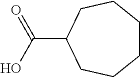 |
| 176 | 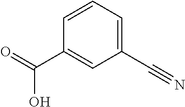 [SMILES: N#Cc1cccc(C(=O)O)c1] |
| 177 | 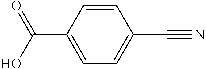 [SMILES: N#Cc1ccc(C(=O)O)cc1] |
| 178 | 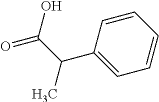 |
| 179 | 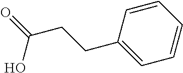 [SMILES: O=C(O)CCc1ccccc1] |
| 180 | 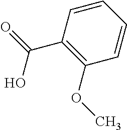 |
| 181 | 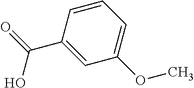 |
| 182 | 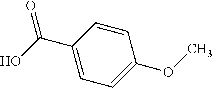 |
| 183 | 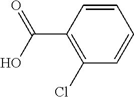 |
| 184 | 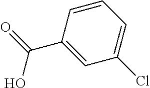 |
| 185 | 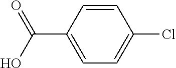 |
| 186 | 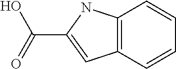 [SMILES: O=C(O)c1cc2ccccc2[nH]1] |
| 187 | 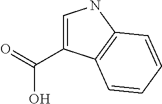 [SMILES: O=C(O)c1c[nH]c2ccccc12] |
| 188 | 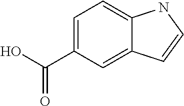 [SMILES: O=C(O)c1ccc2[nH]ccc2c1] |
| 189 | 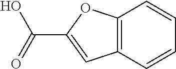 [SMILES: O=C(O)c1cc2ccccc2o1] |
| 190 | 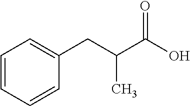 |
| 191 | 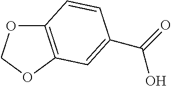 [SMILES: O=C(O)c1ccc2c(c1)OCO2] |
| 192 | 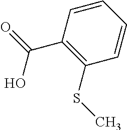 |
| 193 | 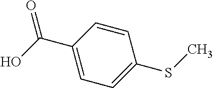 |
| 194 | 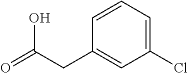 [SMILES: O=C(O)Cc1cccc(Cl)c1] |
| 195 | 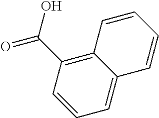 [SMILES: O=C(O)c1cccc2ccccc12] |
| 196 | 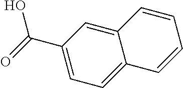 [SMILES: O=C(O)c1ccc2ccccc2c1] |
| 197 | 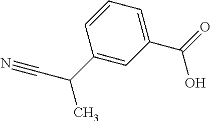 |
| 198 | 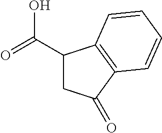 [SMILES: O=C1CC(C(=O)O)c2ccccc21] |
| 199 | 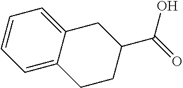 [SMILES: O=C(O)C1CCc2ccccc2C1] |
| 200 | 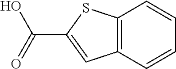 [SMILES: O=C(O)c1cc2ccccc2s1] |
| 201 | 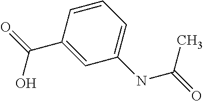 |
| 202 | 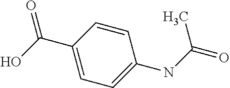 |
| 203 | 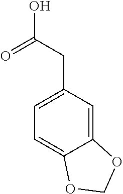 [SMILES: O=C(O)Cc1ccc2c(c1)OCO2] |
| 204 | 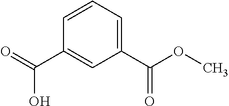 |
| 205 | 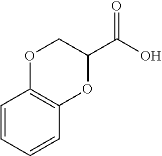 [SMILES: O=C(O)C1COc2ccccc2O1] |
| 206 | 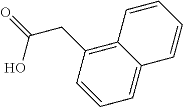 [SMILES: O=C(O)Cc1cccc2ccccc12] |
| 207 | 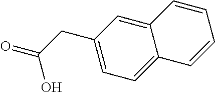 [SMILES: O=C(O)Cc1ccc2ccccc2c1] |
| 208 | 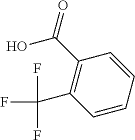 [SMILES: O=C(O)c1ccccc1C(F)(F)F] |
| 209 | 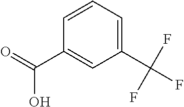 [SMILES: O=C(O)c1cccc(C(F)(F)F)c1] |
| 210 | 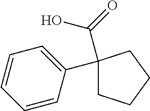 [SMILES: O=C(O)C1(c2ccccc2)CCCC1] |
| 211 | 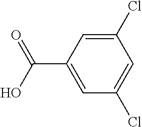 [SMILES: O=C(O)c1cc(Cl)cc(Cl)c1] |
| 212 | 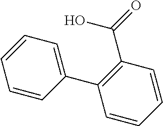 [SMILES: O=C(O)c1ccccc1-c1ccccc1] |
| 213 | 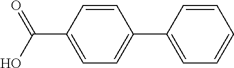 [SMILES: O=C(O)c1ccc(-c2ccccc2)cc1] |
| 214 | 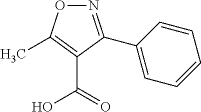 |
| 215 | 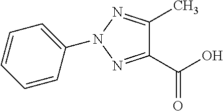 |
| 216 | 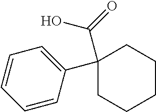 [SMILES: O=C(O)C1(c2ccccc2)CCCCC1] |
| 217 | 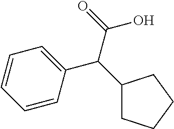 [SMILES: O=C(O)C(c1ccccc1)C1CCCC1] |
| 218 | 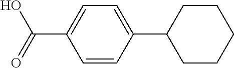 [SMILES: O=C(O)c1ccc(C2CCCCC2)cc1] |
| 219 | 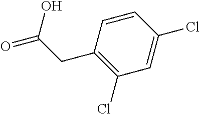 [SMILES: O=C(O)Cc1ccc(Cl)cc1Cl] |
| 220 | 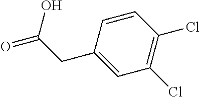 [SMILES: O=C(O)Cc1ccc(Cl)c(Cl)c1] |
| 221 | 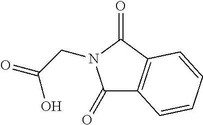 [SMILES: O=C(O)CN1C(=O)c2ccccc2C1=O] |
| 222 | 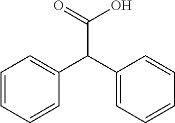 [SMILES: O=C(O)C(c1ccccc1)c1ccccc1] |
| 223 | 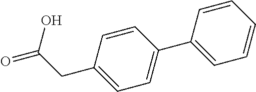 [SMILES: O=C(O)Cc1ccc(-c2ccccc2)cc1] |
| 224 | 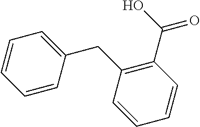 [SMILES: O=C(O)c1ccccc1Cc1ccccc1] |
| 225 | 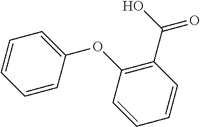 [SMILES: O=C(O)c1ccccc1Oc1ccccc1] |
| 226 | 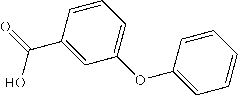 [SMILES: O=C(O)c1cccc(Oc2ccccc2)c1] |
| 227 | 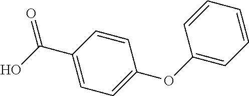 [SMILES: O=C(O)c1ccc(Oc2ccccc2)cc1] |
| 228 | 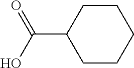 |
| 229 | 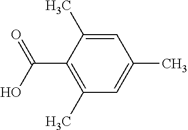 |
| 230 | 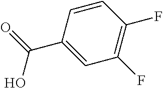 [SMILES: O=C(O)c1ccc(F)c(F)c1] |
| 231 | 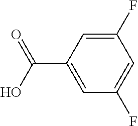 [SMILES: O=C(O)c1cc(F)cc(F)c1] |
| 232 | 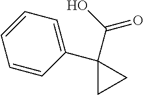 [SMILES: O=C(O)C1(c2ccccc2)CC1] |
| 233 | 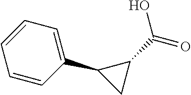 [SMILES: O=C(O)[C@@H]1C[C@H]1c1ccccc1] |

### Preparation of Examples 234-246

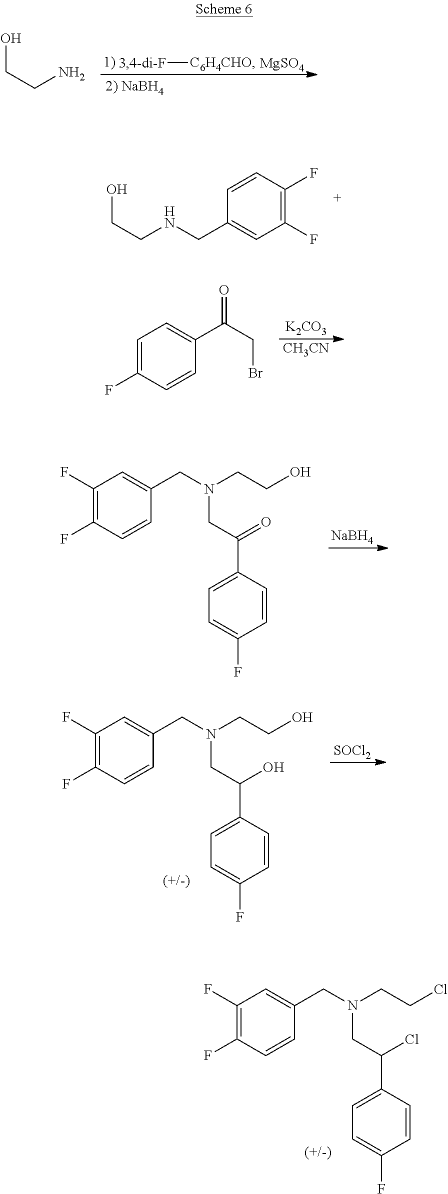

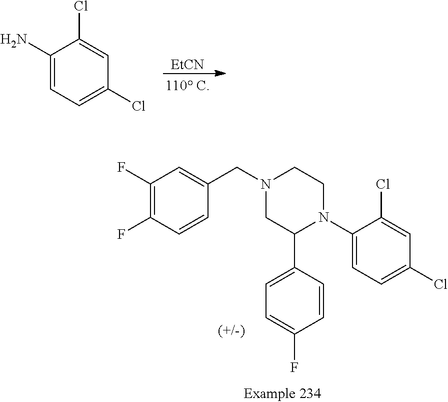 [SMILES: "EtCN" ; Nc1ccc(Cl)cc1Cl ; Fc1ccc(C2CN(Cc3ccc(F)c(F)c3)CCN2c2ccc(Cl)cc2Cl)cc1]

### Step 1:

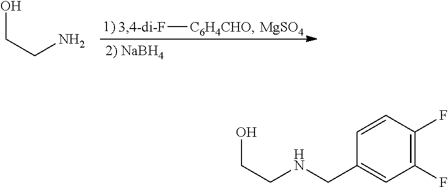

Ethanolamine (3 g), 3,4-difluorobenzaldehyde (7 g), and MgSO₄ (15 g) were taken up in CH₂Cl₂ and stirred at 25° C. (4 h). The solution was filtered and concentrated, thereby providing the imine as a thick oil. The imine was taken up MeOH and cooled to 0° C. Sodium borohydride (1.9 g) was added in portions at 0° C. After the addition of NaBH₄, the reaction was warmed to 25° C. and stirred for 0.5 h. The reaction mixture was quenched with 1 N NCl_{(aq.)}. The mixture was concentrated to remove MeOH. The residue was extracted with Et₂O. The aqueous layer was cooled to 0° C. and made basic via addition of NaOH pellets (pH=11-12). The aqueous layer was extracted with CH₂Cl₂. The combined CH₂Cl₂ layers were dried (MgSO₄), filtered, and concentrated to furnish the amino-alcohol (6.5 g, 70%) as a thick oil.

### Step 2:

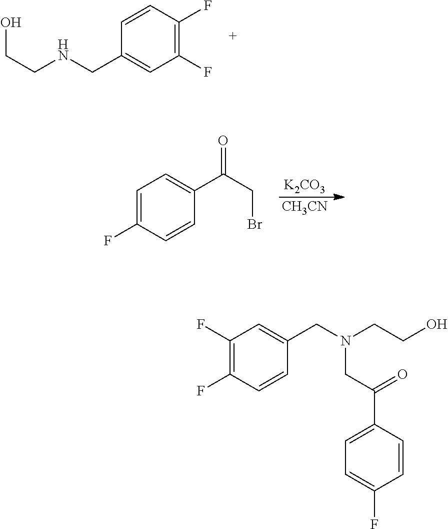

The amino-alcohol (390 mg), bromo-ketone (500 mg), and K₂CO₃ (380 mg) were taken up in CH₃CN and stirred at 25° C. (19 h). The solution was concentrated. The residue was partitioned between EtOAc and water. The aqueous layer was extracted with EtOAc. The combined EtOAc layers were washed with brine, dried (MgSO₄), and filtered. Concentration gave a yellow oil. Purification via thin-layer preparative chromatography (10% EtOAc in CH₂Cl₂, SiO₂) gave 610 mg (90%) of the keto-alcohol as an oil.

### Step 3:

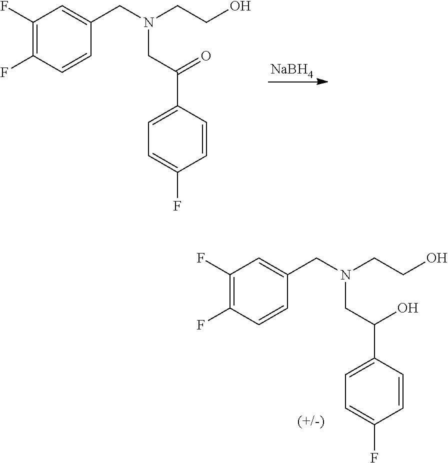 [SMILES: O=C(CN(CCO)Cc1ccc(F)c(F)c1)c1ccc(F)cc1 ; "NaBH4" ; OCCN(Cc1ccc(F)c(F)c1)CC(O)c1ccc(F)cc1]

The keto-alcohol (610 mg) was taken up in MeOH. Sodium borohydride (90 mg) was added, and the solution was stirred at 25° C. (20 h). The solution was quenched with NaHCO_{3(aq)} and concentrated to remove MeOH. The mixture was partitioned between EtOAc and water. The aqueous layer was extracted with EtOAc. The combined organic layers were washed with brine, dried (MgSO₄), filtered, and concentrated to afford 540 mg (88%) of the diol as a colorless oil.

### Step 4:

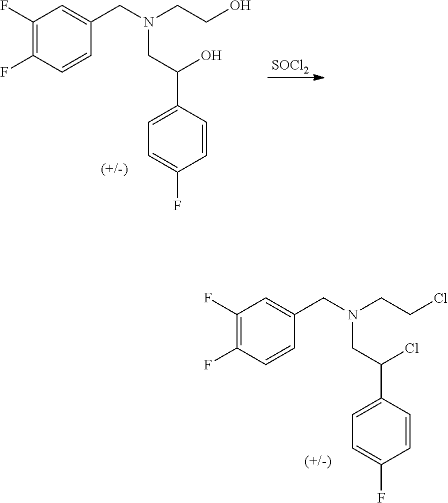

The diol (540 mg) and SOCl₂ (493 mg) were taken up in DCE and refluxed for 4 h (85° C.). The solution was diluted with CH₂Cl₂ and washed with saturated NaHCO_{3(aq)}. The aqueous layer was extracted with CH₂Cl₂. The combined organic layers were dried (MgSO₄), filtered, and concentrated to give the di-chloro-amine as a yellow oil. This material was used without any further purification.

### Step 5:

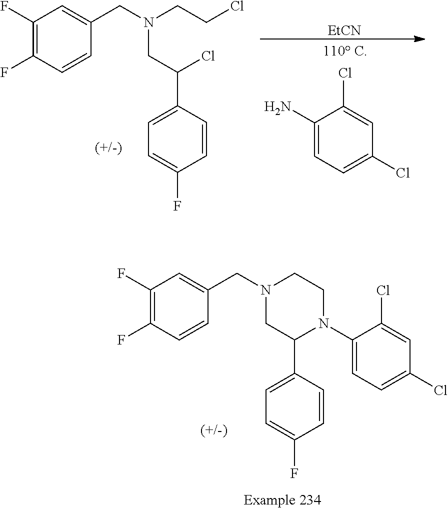 [SMILES: "EtCN" ; Fc1ccc(C(Cl)CN(CCCl)Cc2ccc(F)c(F)c2)cc1 ; Nc1ccc(Cl)cc1Cl ; Fc1ccc(C2CN(Cc3ccc(F)c(F)c3)CCN2c2ccc(Cl)cc2Cl)cc1]

The dichloro-amine (200 mg) and 2,4-dichloroaniline (269 mg) were taken up in EtCN (i.e., propionitrile) and heated at 110° C. (18 h). The solution was concentrated. The residue was partitioned between EtOAc and saturated NaHCO_{3(aq)}. The aqueous layer was extracted with EtOAc. The combined organic layers were washed with brine and dried (MgSO₄). Filtration and concentration gave a yellow oil. Purification via thin-layer preparative chromatography (15% EtOAc in hexanes, SiO₂) gave 24 mg (10%) of Example 234 as a colorless oil.

The following Examples were prepared following the same procedures described in Scheme 6 using the appropriate reagents outlined in Table IV. The bromoketones of Table IV are commercially available, e.g. from Aldrich or Acros.

TABLE IV

| | Bromo-Ketone | Aniline | |
|---|---|---|---|
| Example | Step 2 | Step 5 | Structure |
| 235 | 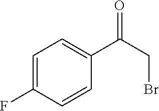 [SMILES: O=C(CBr)c1ccc(F)cc1] | 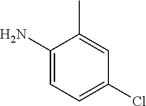 | 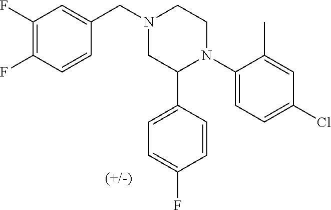 [SMILES: Cc1cc(Cl)ccc1N1CCN(Cc2ccc(F)c(F)c2)CC1c1ccc(F)cc1] |
| 236 | 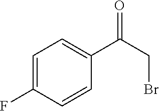 [SMILES: O=C(CBr)c1ccc(F)cc1] | 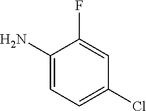 | 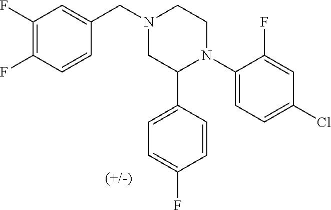 [SMILES: Fc1ccc(C2CN(Cc3ccc(F)c(F)c3)CCN2c2ccc(Cl)cc2F)cc1] |
| 237 | 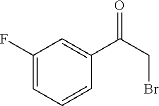 [SMILES: O=C(CBr)c1cccc(F)c1] | 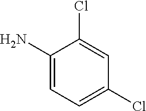 | 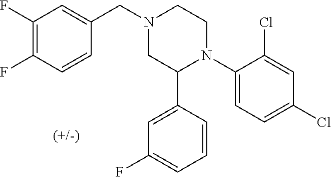 [SMILES: Fc1cccc(C2CN(Cc3ccc(F)c(F)c3)CCN2c2ccc(Cl)cc2Cl)c1] |
| 238 | 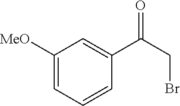 [SMILES: COc1cccc(C(=O)CBr)c1] | 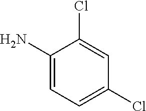 | 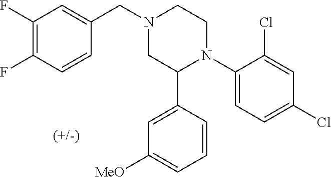 [SMILES: COc1cccc(C2CN(Cc3ccc(F)c(F)c3)CCN2c2ccc(Cl)cc2Cl)c1] |
| 239 | 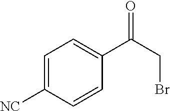 [SMILES: N#Cc1ccc(C(=O)CBr)cc1] | 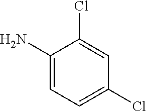 | 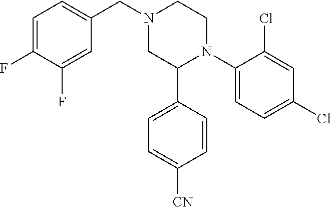 [SMILES: N#Cc1ccc(C2CN(Cc3ccc(F)c(F)c3)CCN2c2ccc(Cl)cc2Cl)cc1] |
| 240 | 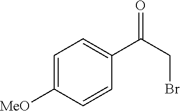 [SMILES: COc1ccc(C(=O)CBr)cc1] | 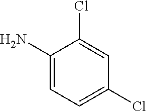 | 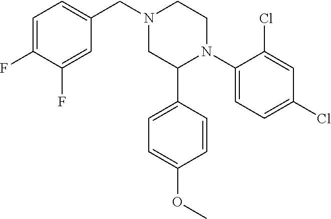 [SMILES: COc1ccc(C2CN(Cc3ccc(F)c(F)c3)CCN2c2ccc(Cl)cc2Cl)cc1] |
| 241 | 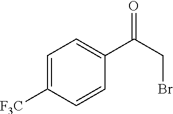 [SMILES: O=C(CBr)c1ccc(C(F)(F)F)cc1] | 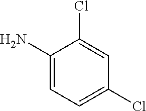 | 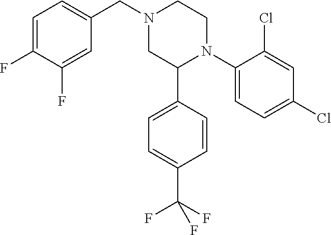 [SMILES: Fc1ccc(CN2CCN(c3ccc(Cl)cc3Cl)C(c3ccc(C(F)(F)F)cc3)C2)cc1F] |
| 242 | 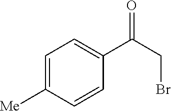 [SMILES: Cc1ccc(C(=O)CBr)cc1] | 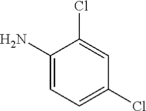 | 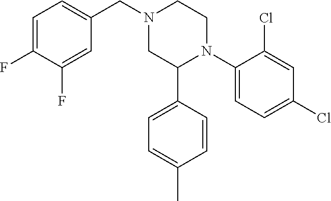 [SMILES: Cc1ccc(C2CN(Cc3ccc(F)c(F)c3)CCN2c2ccc(Cl)cc2Cl)cc1] |
| 243 | 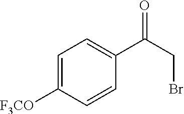 [SMILES: O=C(CBr)c1ccc(OC(F)(F)F)cc1] | 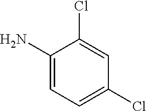 | 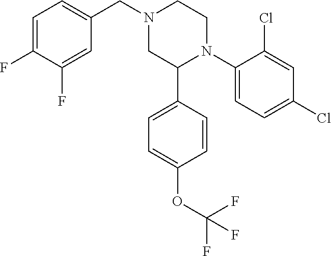 [SMILES: Fc1ccc(CN2CCN(c3ccc(Cl)cc3Cl)C(c3ccc(OC(F)(F)F)cc3)C2)cc1F] |
| 244 | 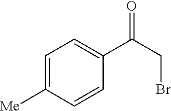 [SMILES: Cc1ccc(C(=O)CBr)cc1] | 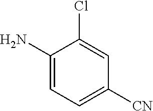 | 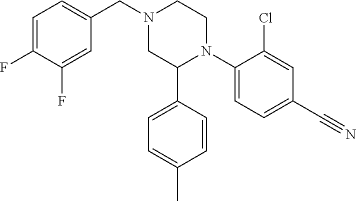 [SMILES: Cc1ccc(C2CN(Cc3ccc(F)c(F)c3)CCN2c2ccc(C#N)cc2Cl)cc1] |
| 245 | 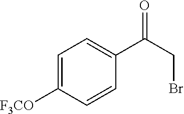 [SMILES: O=C(CBr)c1ccc(OC(F)(F)F)cc1] | 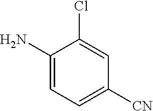 | 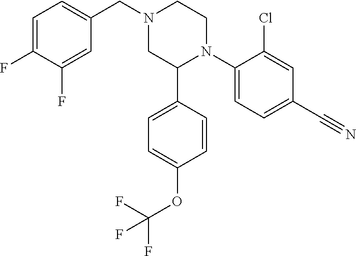 [SMILES: N#Cc1ccc(N2CCN(Cc3ccc(F)c(F)c3)CC2c2ccc(OC(F)(F)F)cc2)c(Cl)c1] |
| 246 | 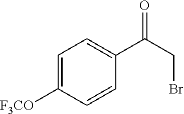 [SMILES: O=C(CBr)c1ccc(OC(F)(F)F)cc1] | 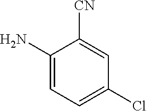 | 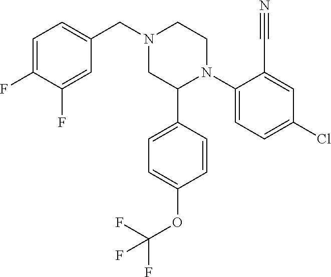 [SMILES: N#Cc1cc(Cl)ccc1N1CCN(Cc2ccc(F)c(F)c2)CC1c1ccc(OC(F)(F)F)cc1] |

### Preparation of Example 247

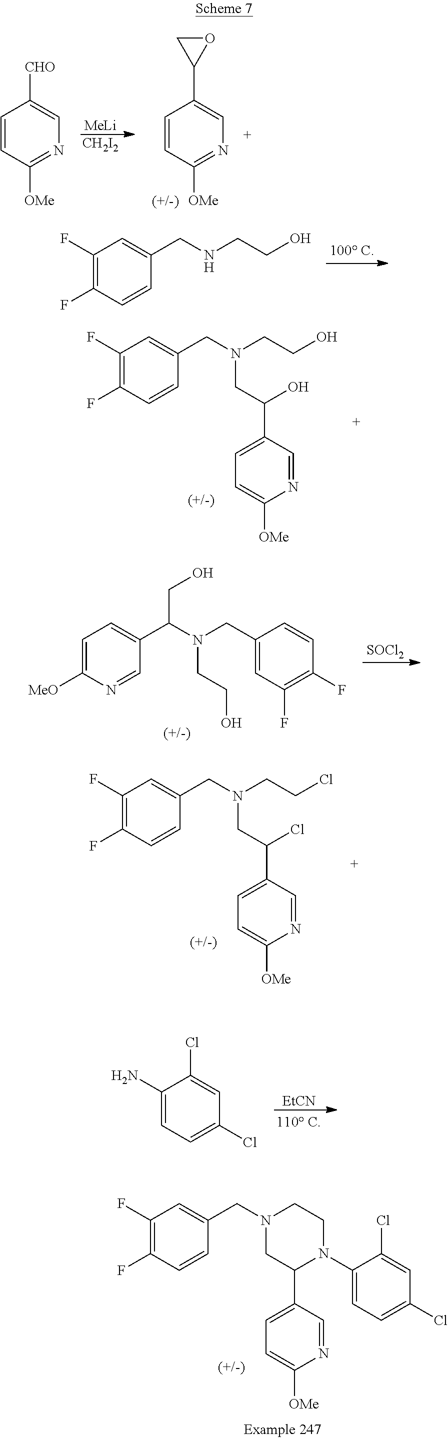

### Step 1:

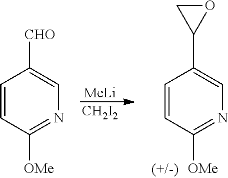

The aldehyde (2 g) and CH₂I₂ (1.8 mL) were taken up in THF (40 mL) and cooled to 0° C. Methyllithium-LiBr complex (20 mL of a 1.5 M solution in Et₂O) was added dropwise to the reaction. The mixture was stirred at 0° C. for 1 h and then at 25° C. for 1 h. The mixture was poured into ice. The mixture was extracted with EtOAc. The combined organic layers were washed with brine and dried (MgSO₄). Filtration and concentration gave the epoxide (2.2 g, 100%) as a yellow oil.

### Step 2:

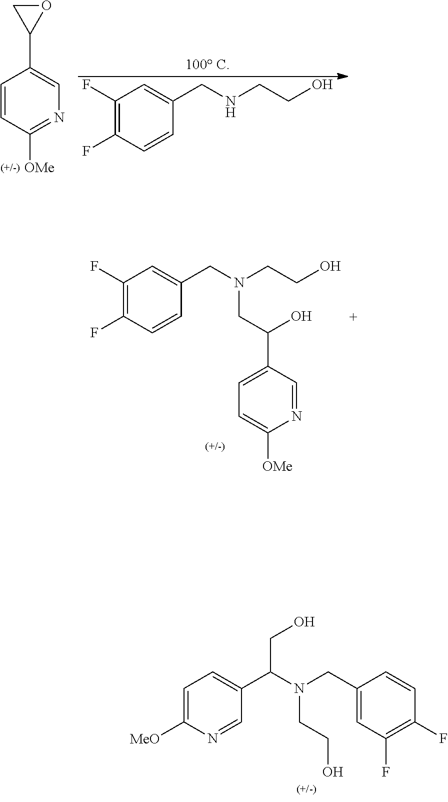

The epoxide (2.4 g) and amino-alcohol (2.97 g) were heated neat at 100° C. (18 h). The residue was purified via flash chromatography (3/1 CH₂Cl₂/acetone, SiO₂) to give 3 g (58%) of the diols as a mixture of isomers.

### Step 3:

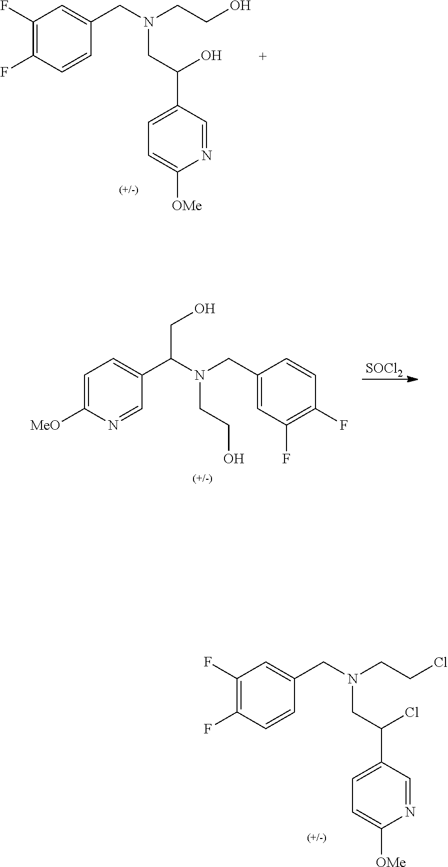 [SMILES: COc1ccc(C(O)CN(CCO)Cc2ccc(F)c(F)c2)cn1 ; COc1ccc(C(CO)N(CCO)Cc2ccc(F)c(F)c2)cn1 ; "SOCl2" ; COc1ccc(C(Cl)CN(CCCl)Cc2ccc(F)c(F)c2)cn1]

The mixture of diols (250 mg) and SOCl₂ (0.2 mL) were taken up in DCE and heated at 70° C. (45 min). The solution was cooled and partitioned between CH₂Cl₂ and 1 N NaOH_{(aq.)}. The aqueous layer was extracted with CH₂Cl₂. The combined organic layers were dried (MgSO₄), filtered, and concentrated to give 187 mg (67%) of the dichloro-amine as a yellow oil. This material was used without any further purification.

### Step 4:

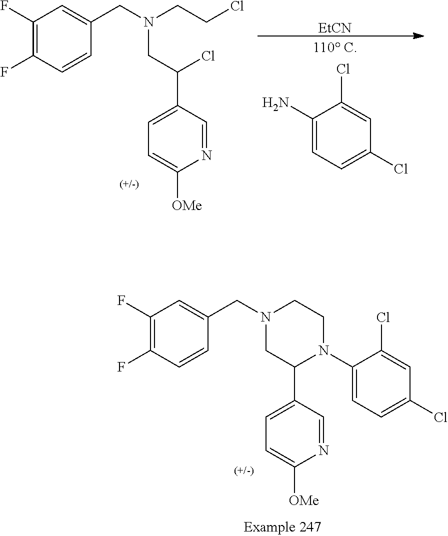 [SMILES: "EtCN" ; COc1ccc(C(Cl)CN(CCCl)Cc2ccc(F)c(F)c2)cn1 ; Nc1ccc(Cl)cc1Cl ; COc1ccc(C2CN(Cc3ccc(F)c(F)c3)CCN2c2ccc(Cl)cc2Cl)cn1]

The dichloro-amine (187 mg), 2,4-dichloroaniline (242 mg), and NaI (50 mg) were taken up in EtCN and heated at 100° C. (19 h). The solution was concentrated. The residue was partitioned between CH₂Cl₂ and 1 N NaOH_{(aq.)}. The aqueous layer was extracted with CH₂Cl₂. The combined organic layers were dried (MgSO₄), filtered, and concentrated. The material contained the desired product and the uncyclized intermediate. The residue was taken up in EtCN and NaI (50 mg) was added. The solution was heated at 100° C. (18 h). The solution was worked up as before. Purification via thin-layer preparative chromatography (9/1 hexanes/acetone, SiO₂) gave 47 mg (20%) of Example 247 as a yellow oil.

### Preparation of Example 248

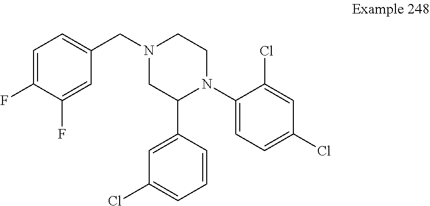 [SMILES: Fc1ccc(CN2CCN(c3ccc(Cl)cc3Cl)C(c3cccc(Cl)c3)C2)cc1F]

The piperazine Example 248 was prepared in a manner similar to the method used to prepare Example 247 except that 3-chlorostyrene oxide (prepared as in step 1, Scheme 7) was used instead of the pyridyl epoxide in step 2 of Scheme 7, above.

### Preparation of Example 249

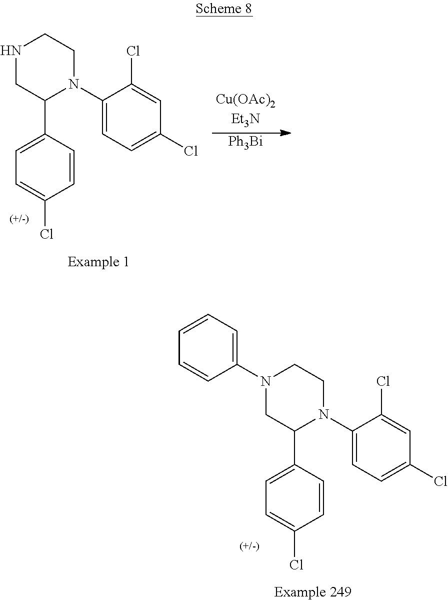 [SMILES: Clc1ccc(C2CNCCN2c2ccc(Cl)cc2Cl)cc1 ; "Ph3Bi" ; Clc1ccc(C2CN(c3ccccc3)CCN2c2ccc(Cl)cc2Cl)cc1]

The NH piperazine Example 1 (100 mg), Ph₃Bi (385 mg), Cu(OAc)₂ (106 mg), and Et₃N (0.12 mL) were taken up in toluene and heated at 115° C. (18 h). The solution was filtered and concentrated. Purification via thin-layer preparative chromatography (10/1 hexanes/Et₂O, SiO₂) gave 78 mg (64%) of Example 249 as a white solid.

### Preparation of Example 250

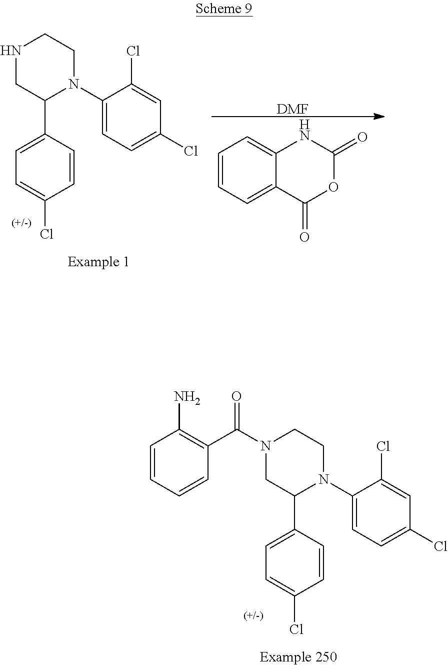

The NH piperazine Example 1 (540 mg) and isatoic anhydride (410 mg) were stirred at 25° C. (18 h). More isatoic anhydride was added (400 mg), and the mixture was stirred at 60° C. (18 h). The solution was partitioned between EtOAc and water. The aqueous layer was extracted with EtOAc. The combined organic layers were washed with brine and dried (MgSO₄). Filtration and concentration gave a yellow oil. Purification via flash chromatography (1/1 EtOAc/hexanes, SiO₂) gave 268 mg (37%) of Example 250 as a white solid.

### Preparation of Examples 251-253

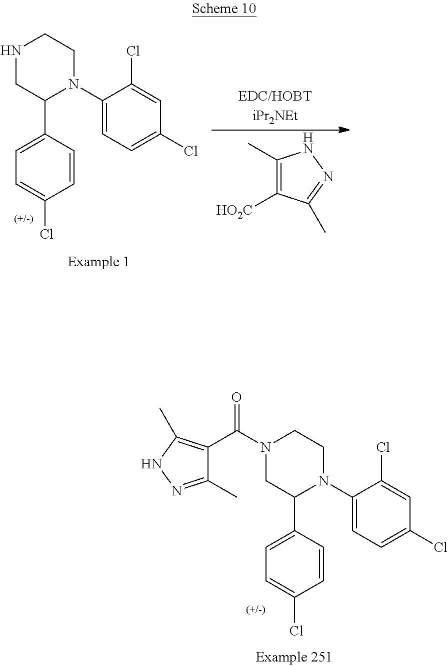

The NH piperazine Example 1 (220 mg), EDC (i.e., 1-(3-dimethylaminopropyl)-3-ethylcarbodiimide hydrochloride) (245 mg), HOBT (i.e., 1-hydroxybenzotriazole) (172 mg), acid (108 mg), and iPr₂NEt (206 mg) were taken up in CH₃CN and stirred at 25° C. (18 h). The solution was partitioned between EtOAc and 1 N NaOH_{(aq.)}. The aqueous layer was extracted with EtOAc. The combined organic layers were washed with brine and dried (MgSO₄). Filtration and concentration gave a solid. The solid was triturated with Et₂O. The white solid was collected and dried to give 125 mg (42%) of Example 251.

The following examples were prepared according to the procedure described in Scheme 10 using the appropriate reagents. The carboxylic acids in Table V were prepared by methods described in WO 00/66558 or U.S. Pat. No. 6,391,865, both of which are herein incorporated by reference in their entirety.

TABLE V

| | Carboxylic | |
|---|---|---|
| Example | Acid | Structure |
| 252 | 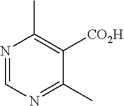 | 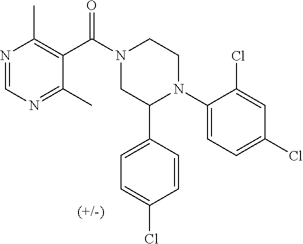 [SMILES: Cc1ncnc(C)c1C(=O)N1CCN(c2ccc(Cl)cc2Cl)C(c2ccc(Cl)cc2)C1] |
| 253 | 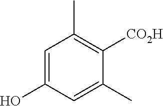 | 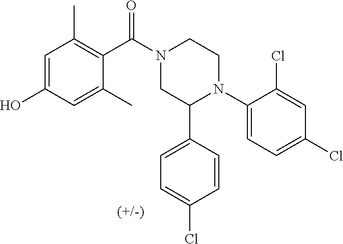 [SMILES: Cc1cc(O)cc(C)c1C(=O)N1CCN(c2ccc(Cl)cc2Cl)C(c2ccc(Cl)cc2)C1] |

### Preparation of Example 254

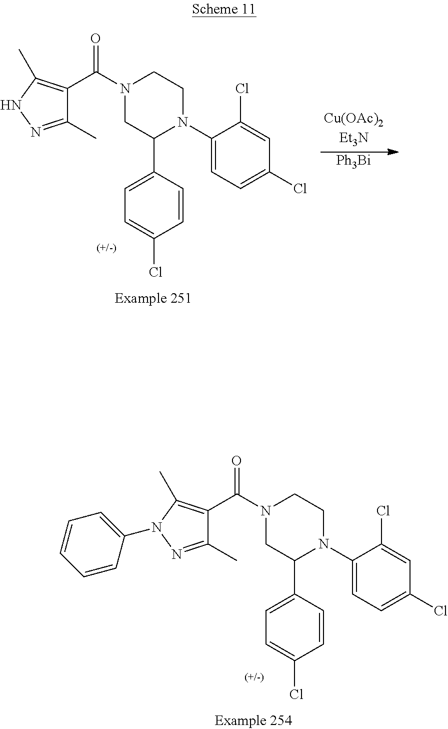 [SMILES: Cc1n[nH]c(C)c1C(=O)N1CCN(c2ccc(Cl)cc2Cl)C(c2ccc(Cl)cc2)C1 ; "Ph3Bi" ; Cc1nn(-c2ccccc2)c(C)c1C(=O)N1CCN(c2ccc(Cl)cc2Cl)C(c2ccc(Cl)cc2)C1]

Example 254 was prepared from Example 251 using the procedure described above in Scheme 8.

### Preparation of Example 255

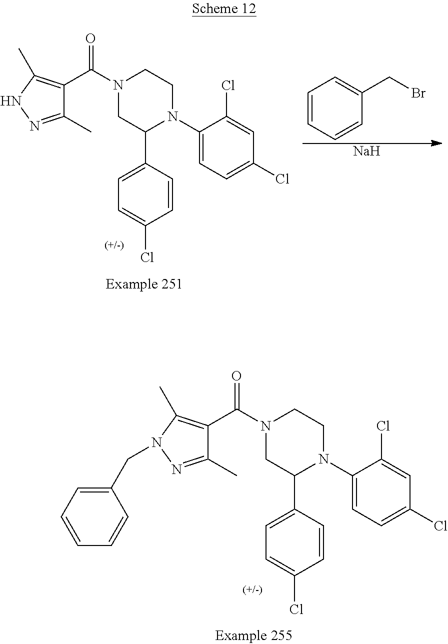

Example 251 (40 mg) was taken up in THF at 25° C. NaH(15 mg of a 60 wt % dispersion in oil) was added. After 10-15 minutes, benzyl bromide (30 mg) was added, and the solution was stirred at 25° C. (18 h). The solution was concentrated, and the residue was partitioned between EtOAc and water. The aqueous layer was extracted with EtOAc. The combined organic layers were washed with brine and dried (MgSO₄). Filtration and concentration gave a yellow oil. Purification via thin-layer preparative chromatography (5% MeOH in CH₂Cl₂, SiO₂) gave 14 mg (29%) of the N-benzyl analog Example 255 as an oil.

### Preparation of Example 256

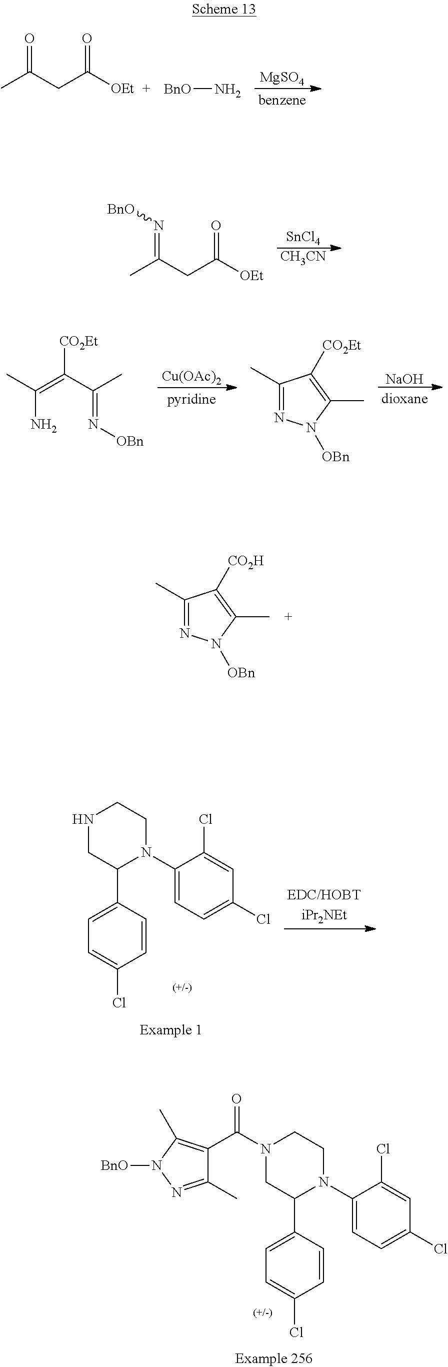

### Step 1:

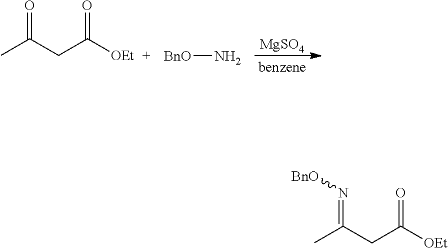

Ethyl acetoacetate (7.5 g, 58 mmol) and O-Benzyl hydroxylamine (7.1 g, 58 mmol), and MgSO₄ (5 g) were taken up in benzene and stirred at 25° C. for 24 hours. Filtration and concentration gave the oxime.

### Step 2:

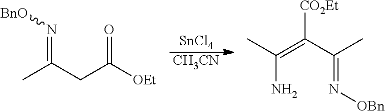

The oxime (1.0 g, 4.25 mmol) was taken up in CH₃CN (8 mL) and cooled to 0° C. SnCl₄ (4.3 ml, 1.0 M in CH₂Cl₂) was added dropwise to the solution at 0° C. The solution was stirred at 0° C. for one hour. The solution was quenched with saturated Na₂CO₃ (aq.). The mixture was extracted with EtOAc. The combined organic layers were washed with brine and dried (MgSO4). Filtration and concentration gave a colorless oil. Purification via flash chromatography (3/1 hexanes/EtOAc, SiO₂) gave 415 mg (35%) of the enamide as a colorless oil.

### Step 3:

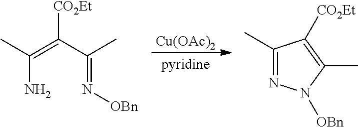

The enamide (415 mg, 1.5 mmol) and Cu(OAc)₂ (400 mg) were taken up in pyridine. The mixture was heated at 100° C. for 4 hours. The solution was cooled and concentrated. The residue was partitioned between EtOAc and 10% NH₄OH_{(aq.)}. The aqueous layer was extracted with EtOAc. The combined organic layers were washed with brine and dried (MgSO₄). Filtration and concentration gave a brown oil. Purification via flash chromatography (9/1 hexanes/EtOAc, SiO₂) gave 330 mg (80%) of the pyrazole as a colorless oil.

### Step 4:

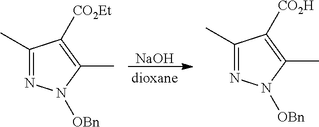

The ester (545 mg, 1.99 mmol) and 1 N NaOH_{(aq.)} was taken up in dioxane/EtOH. The solution was heated at 75° C. for 24 hours. The solution was concentrated. The solution was acidified with 1 M NCl_{(aq.)} (pH=2-3). The resulting white precipitate was collected and dried under high vacuum. The acid was obtained as a white powder (314 mg, 64%). ¹H NMR (CDCl₃, 400 MHz) δ 2.07 (s, 3H), 2.46 (s, 3H), 5.26 (s, 2H), 7.25-7.37 (m, 5H). HRMS calc'd for C₁₃H₁₅O₃N (MH⁺) 247.1083; Found: 247.1089.

### Step 5:

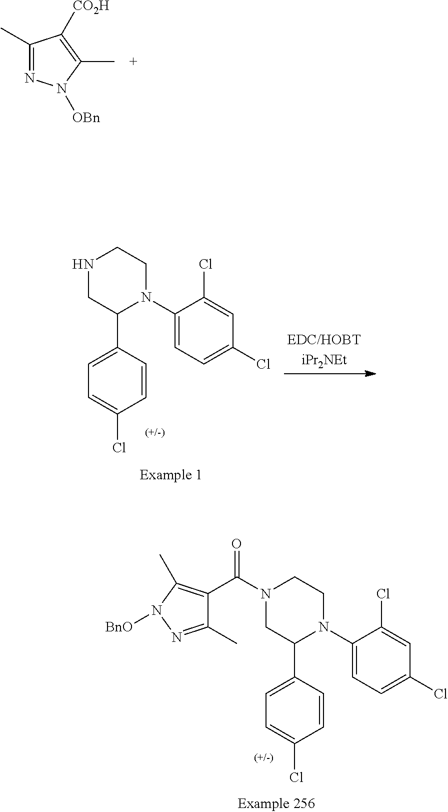

### Preparation of Examples 257-262

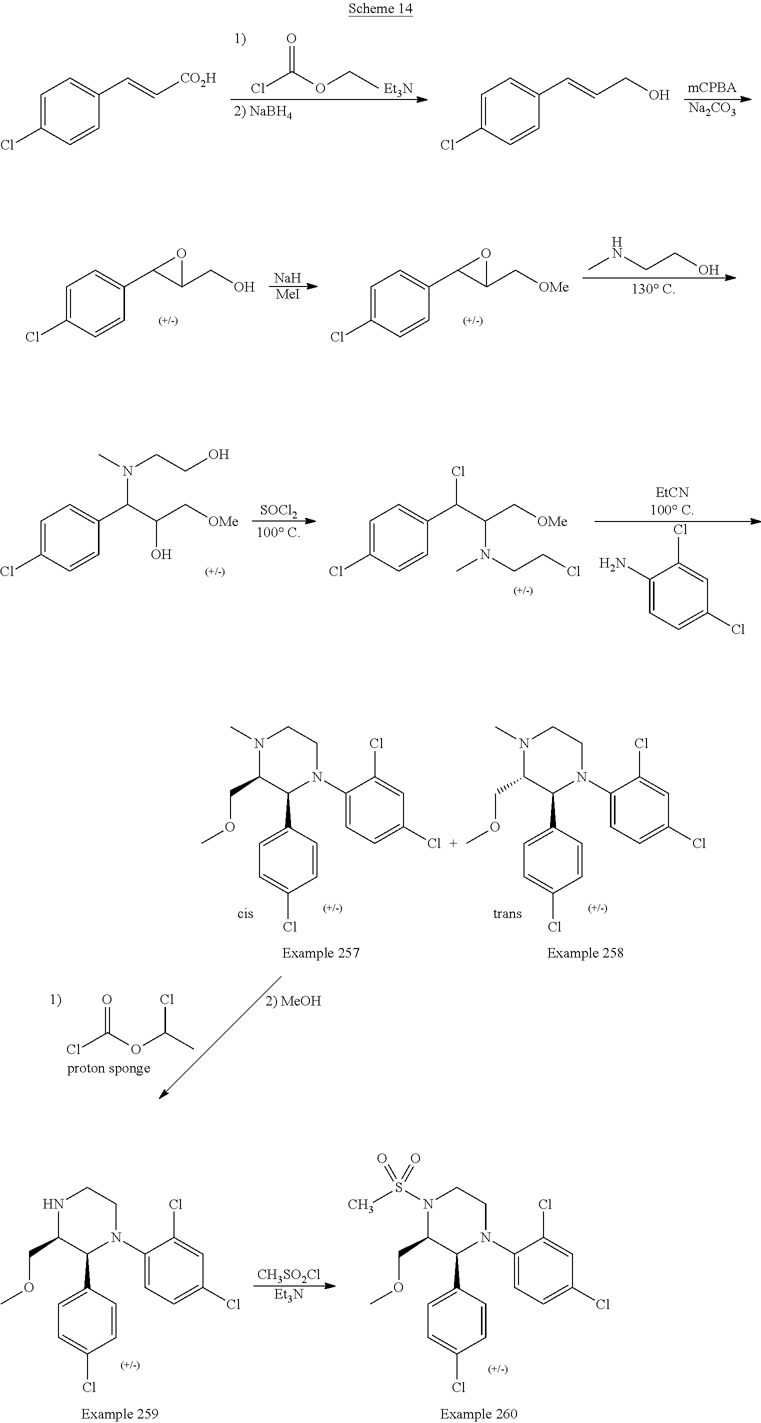

### Step 1:

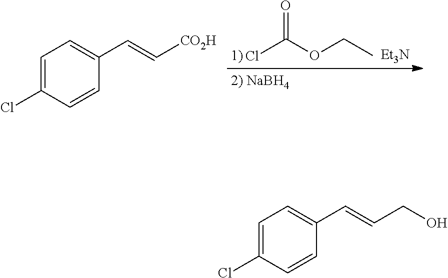

4-Chloro-cinnamic acid (30 g) and Et₃N (18.3 g) were taken up in THF (300 mL) at 0° C. Ethyl chloroformate (17.3 mL) was added dropwise at 0° C. The slurry was stirred at 0° C. for one hour. The Et₃NHCl was removed by filtration, and the filtrate was filtered directly into cold water. The filtrate was washed with cold THF. The solution was placed into an ice-bath, and NaBH₄ (13.2 g) was added in portions (with gas evolution). The ice bath was removed, and the resulting solution was stirred at 25° C. (16 h). The reaction was quenched with 2 M NCl_{(aq.)}. Diethyl ether was added, and the mixture was allowed to stir at 25° C. for 3 hours. The aqueous layer was extracted with Et₂O. The combined organic layers were washed with brine and dried (MgSO₄). Filtration and concentration gave a yellow oil. Purification via flash chromatography (15% EtOAc in CH₂Cl₂, SiO₂) gave 23.5 grams (85% yield) of the alcohol as an oil that slowly solidified.

### Step 2:

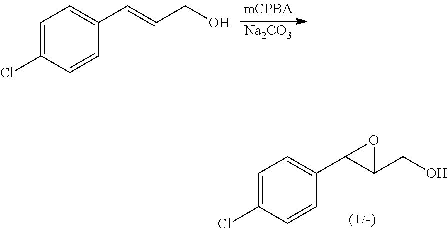

The alcohol (23.5 g) and Na₂CO₃ (17.8 g) were taken up in CH₂Cl₂ (300 mL) and cooled to 0° C. (mechanical stirrer). m-CPBA (i.e. m-chloroperoxybenzoic acid) (38 grams) was added in portions at 0° C. The mixture was warmed to 25° C. and stirred at that temp for 16 hours. The solution was washed with 10% Na₂S₂O_{3(aq.)} and saturated NaHCO_{3(aq)}. The organic layers were dried (MgSO₄), filtered, and concentrated. Purification via flash chromatography (15% EtOAc in CH₂Cl₂, SiO₂) gave 16.5 grams (64%) of the racemic epoxide as an oil.

### Step 3:

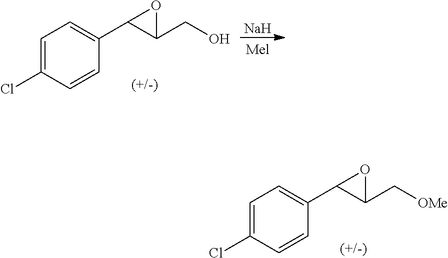

Sodium hydride (4.3 g) was suspended in DMF (100 mL) at −20° C. The epoxy-alcohol (16.5 g) was added, and the reaction mixture was stirred at −20° C. for 0.5 h. Iodomethane (19 g) was added at −20° C., and the resulting reaction mixture was stirred at that temperature for 40 minutes. The reaction mixture was allowed to warm to 25° C. and stir at that temperature for 3 hours. The reaction mixture was poured into a mixture of EtOAc and water. The aqueous layer was extracted with EtOAc. The combined organic layers were washed with brine, dried (MgSO₄), and filtered which furnished the methyl ether (14.6 g, 82%). This material was used without any further purification.

### Step 4:

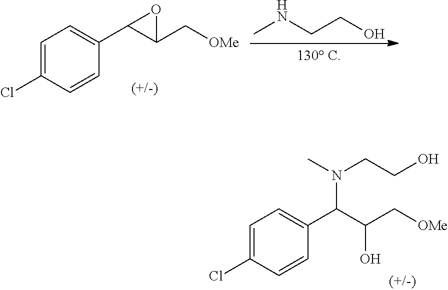

The methyl ether (14.6 g) and N-methyl-ethanolamine (5.5 g) were heated neat (130° C., 24 hours). Purification via flash chromatography (CH₂Cl₂, EtOAc, then 20% MeOH in EtOAc, SiO₂) furnished the diol (15 g, 75%) as a viscous oil.

### Step 5:

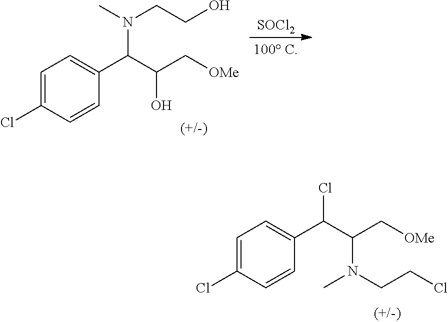

The diol (7 g) and SOCl₂ (4.7 mL) were taken up in DCE (50 mL) and the solution was heated to 100° C. (3 h). The solution was diluted with CH₂Cl₂ and slowly quenched with saturated NaHCO_{3(aq)}. The aqueous layer was extracted with CH₂Cl₂. The combined organic layers were dried (MgSO₄), filtered, and concentrated to furnish the di-chloro amine (7.2 g, 91%). This material was used without any further purification.

### Step 6:

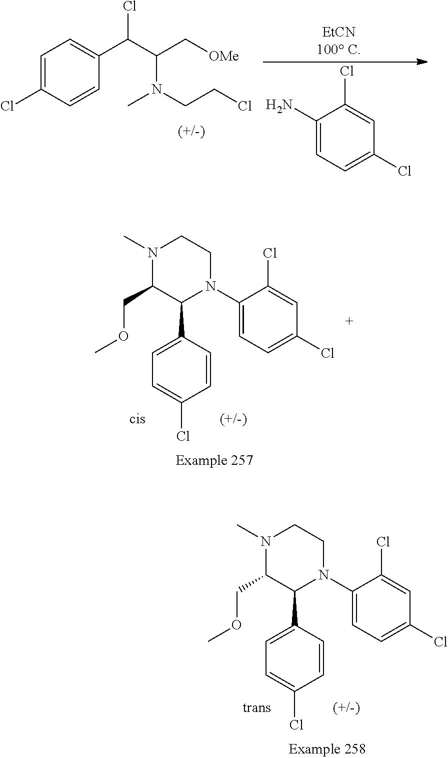

The dichloro-amine (7.2 g) and 2,4-dichloroaniline (11.5 g) were heated in propionitrile (100° C., 24 h). Solution was evaporated. The residue was partitioned between EtOAc and 2 N NaOH_{(aq.)}. The aqueous layer was extracted with EtOAc. The combined organic layers were washed with brine, dried (MgSO₄), and filtered. Purification via flash chromatography twice (2% MeOH in CH₂Cl₂ then 10% acetone in CH₂Cl₂, SiO₂) furnished the cis and trans piperazines, Examples 257 and 258, (8.05 grams, 85% 10/1 cis/trans ratio) as thick oils that solidified on standing.

### Step 7:

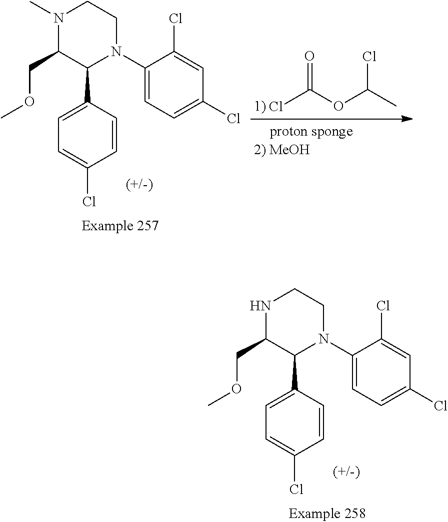

The cis-N-methyl piperazine Example 257 (2 g), proton sponge (i.e., N,N,N′,N′-tetramethylnaphthalene-1,8-diamine) (0.32 g), and 1-chloroethyl-chloroformate (1.4 g) were heated in DCE (90° C., 20 h). The solution was concentrated. The residue was taken up in MeOH and heated at reflux for 7 hours. The solution was evaporated. The residue was partitioned between EtOAc and saturated NaHCO_{3(aq)}. The aqueous layer was extracted with EtOAc. The combined organic layers were washed with brine and dried (MgSO₄). Filtration and concentration gave a yellow oil. Purification via flash chromatography (5% MeOH in CH₂Cl₂, SiO₂) gave the NH piperazine Example 259 (1.3 g, 67%) as a yellow oil.

### Step 8:

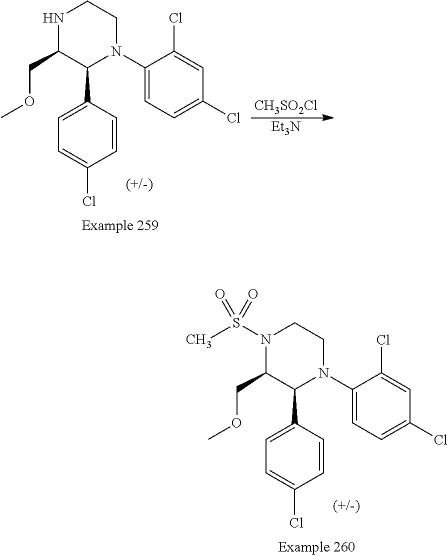

The NH-piperazine Example 259 (100 mg) and Et₃N (34 mg) were taken up in CH₂Cl₂. MeSO₂Cl (35 mg) was added, and the solution was stirred at 25° C. (16 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via thin-layer preparative chromatography (2% EtOAc in CH₂Cl₂, SiO₂) furnished the sulfonamide Example 260 (77 mg, 64%) as a yellow oil.

The following examples were prepared according to Step 8 in Scheme 14 using the appropriate reagent (Table VI).

TABLE VI

| Example | Sulfonyl Chloride | Structure |
|---|---|---|
| 261 | 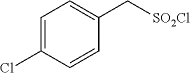 [SMILES: O=S(=O)(Cl)Cc1ccc(Cl)cc1] | 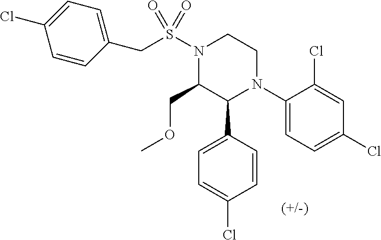 [SMILES: COC[C@@H]1[C@H](c2ccc(Cl)cc2)N(c2ccc(Cl)cc2Cl)CCN1S(=O)(=O)Cc1ccc(Cl)cc1] |
| 262 | 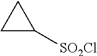 | 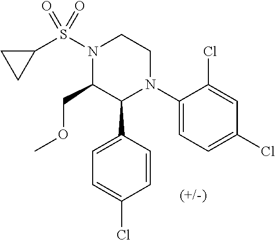 [SMILES: COC[C@@H]1[C@H](c2ccc(Cl)cc2)N(c2ccc(Cl)cc2Cl)CCN1S(=O)(=O)C1CC1] |

### Preparation of Examples 263-266

Examples 263-266 were prepared using procedures similar to those described in Scheme 14, as shown in Scheme 15.

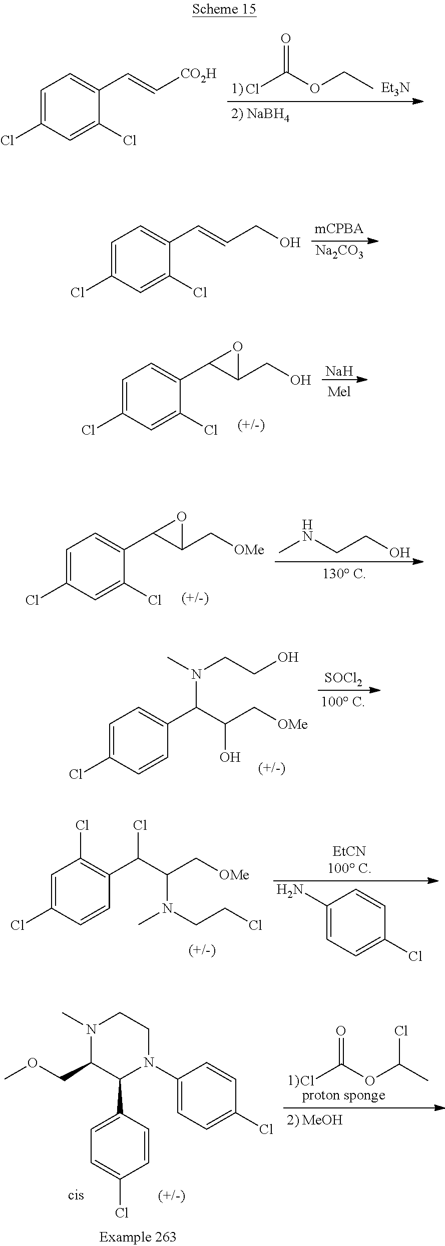

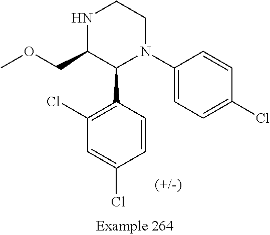 [SMILES: COC[C@H]1NCCN(c2ccc(Cl)cc2)[C@H]1c1ccc(Cl)cc1Cl]

The Examples shown in Table VII were prepared from the appropriate sulfonyl chlorides and the piperazine Example 264 shown in Scheme 15 according to the procedure outlined in Step 8 of Scheme 14.

TABLE VII

| | Sulfonyl | |
|---|---|---|
| | Chloride | |
| Example | Step 7 | Structure |
| 265 | 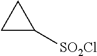 | 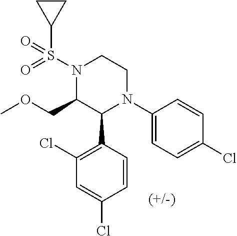 [SMILES: COC[C@@H]1[C@H](c2ccc(Cl)cc2Cl)N(c2ccc(Cl)cc2)CCN1S(=O)(=O)C1CC1] |
| 266 | CH₃SO₂Cl | 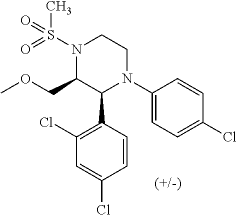 [SMILES: COC[C@@H]1[C@H](c2ccc(Cl)cc2Cl)N(c2ccc(Cl)cc2)CCN1S(C)(=O)=O] |

### Preparation of Examples 267-270

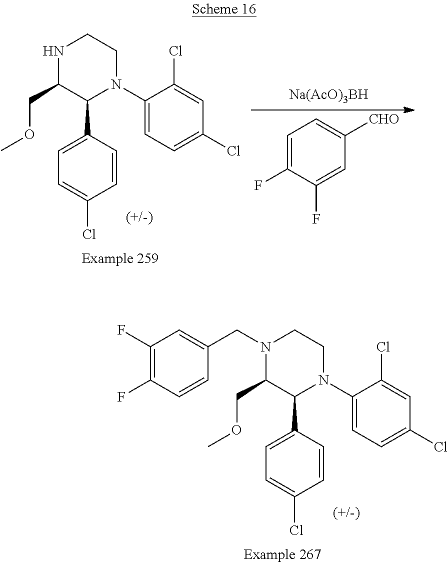

The NH-piperazine Example 259 (100 mg), 3,4-difluorobenzaldehyde (40 mg), and Na(AcO)₃BH (110 mg) were stirred in CH₂Cl₂ at 25° C. (16 h). The solution was diluted with CH₂Cl₂ and washed with 1 N NaOH_{(aq.)}. The aqueous layer was extracted with CH₂Cl₂. The combined organic layers were dried (MgSO₄). Filtration and concentration gave a yellow oil. Purification via preparative thin-layer chromatography (2% EtOAc in CH₂Cl₂, SiO₂) furnished 44 mg (33%) of Example 267 as a colorless oil.

Following the same procedure as described in Scheme 16 the following Example was prepared from the appropriate aldehyde.

TABLE VIII

| Example | Aldehyde | Structure |
|---|---|---|
| 268 | 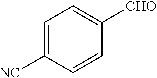 | 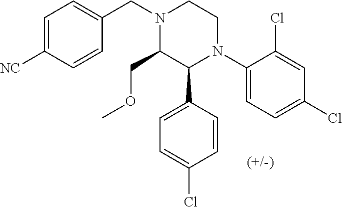 [SMILES: COC[C@@H]1[C@H](c2ccc(Cl)cc2)N(c2ccc(Cl)cc2Cl)CCN1Cc1ccc(C#N)cc1] |

The following Examples were prepared using the piperazine Example 264 of Scheme 15 and the procedure of Scheme 16. The appropriate reagents are listed below (Table IX).

TABLE IX

| Example | Aldehyde | Structure |
|---|---|---|
| 269 | 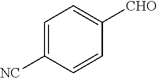 | 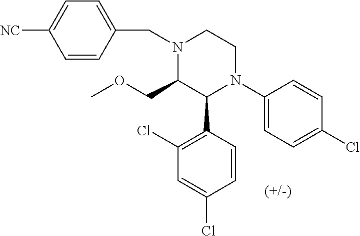 [SMILES: COC[C@@H]1[C@H](c2ccc(Cl)cc2Cl)N(c2ccc(Cl)cc2)CCN1Cc1ccc(C#N)cc1] |
| 270 | 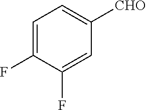 | 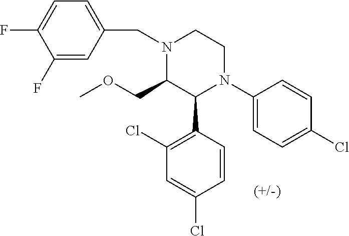 [SMILES: COC[C@@H]1[C@H](c2ccc(Cl)cc2Cl)N(c2ccc(Cl)cc2)CCN1Cc1ccc(F)c(F)c1] |

### Preparation of Examples 271-272

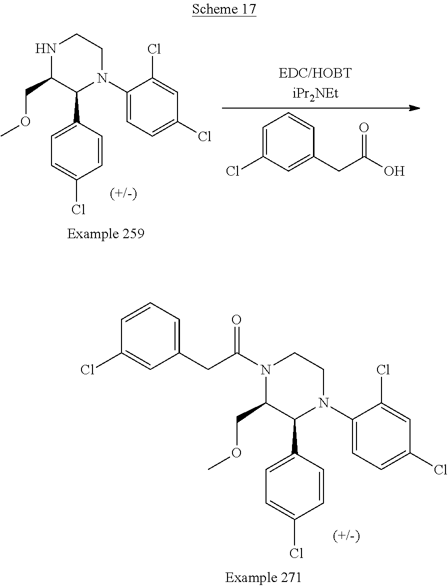 [SMILES: COC[C@H]1NCCN(c2ccc(Cl)cc2Cl)[C@H]1c1ccc(Cl)cc1 ; O=C(O)Cc1cccc(Cl)c1 ; COC[C@@H]1[C@H](c2ccc(Cl)cc2)N(c2ccc(Cl)cc2Cl)CCN1C(=O)Cc1cccc(Cl)c1]

The NH-piperazine Example 259 (100 mg), EDC (99 mg), HOBT (69 mg), iPrNEt (67 mg), and the acid (48 mg) were taken up in CH₂Cl₂. The solution was stirred at 25° C. (16 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via thin-layer preparative chromatography (5% EtOAc in CH₂Cl₂, SiO₂) furnished the amide Example 271 (20 mg, 14%) as a colorless oil.

The following Example was prepared according to the procedures described in Scheme 17 using the piperazine Example 264 shown in Scheme 15. The appropriate reagent is listed below (Table X).

TABLE X

| | Aldehyde | |
|---|---|---|
| Example | Scheme 2 | Structure |
| 272 | 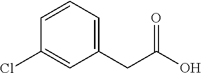 [SMILES: O=C(O)Cc1cccc(Cl)c1] | 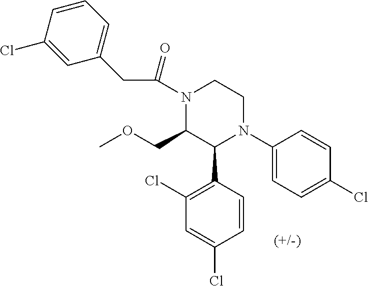 [SMILES: COC[C@@H]1[C@H](c2ccc(Cl)cc2Cl)N(c2ccc(Cl)cc2)CCN1C(=O)Cc1cccc(Cl)c1] |

### Preparation of Examples 273-274

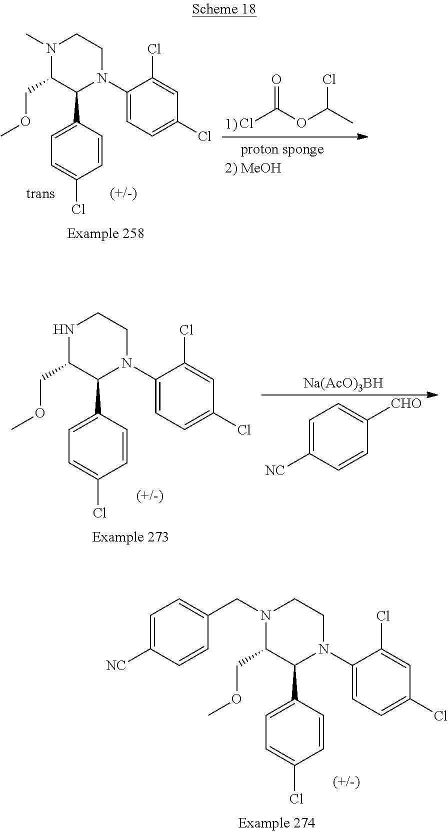

### Step 1:

The trans-piperazine Example 258 was converted into the NH piperazine Example 273 as described previously in Scheme 14, Step 7.

### Step 2:

The NH piperazine Example 273 and 4-cyanobenzaldehyde were reacted following the procedure described in Scheme 16 to provide Example 274.

### Preparation of Example 275

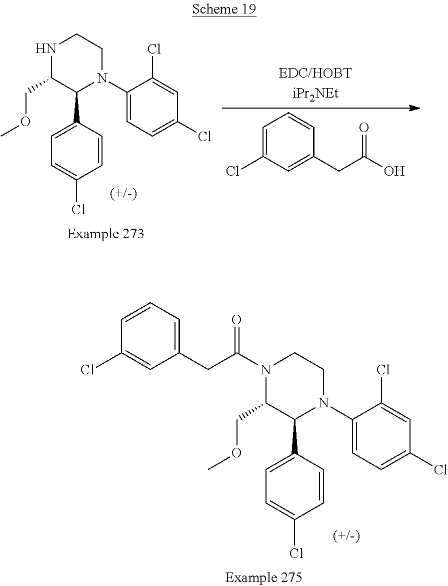 [SMILES: COC[C@@H]1NCCN(c2ccc(Cl)cc2Cl)[C@H]1c1ccc(Cl)cc1 ; O=C(O)Cc1cccc(Cl)c1 ; COC[C@H]1[C@H](c2ccc(Cl)cc2)N(c2ccc(Cl)cc2Cl)CCN1C(=O)Cc1cccc(Cl)c1]

Example 275 was prepared using the procedure described for the corresponding cis isomer (Scheme 17).

### Preparation of Examples 276-277

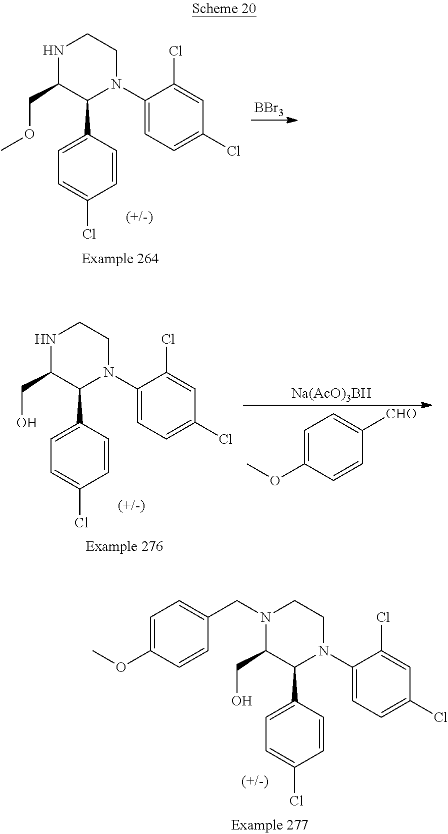

### Step 1:

The NH-piperazine Example 264 (108 mg) was taken up in CH₂Cl₂. Boron tribromide (0.13 mL) was added, and the resulting solution was stirred at 25° C. (16 h). The solution was quenched with saturated NaHCO_{3(aq)}. The aqueous layer was extracted with CH₂Cl₂. The combined organic layers were dried (MgSO₄), filtered, and concentrated. The crude product Example 276 was used without any further purification.

### Step 2:

The amino-alcohol Example 276 (460 mg) and 4-methoxybenzaldehyde were reacted according to the procedure described above (Scheme 18) to furnish Example 277.

### Preparation of Example 278

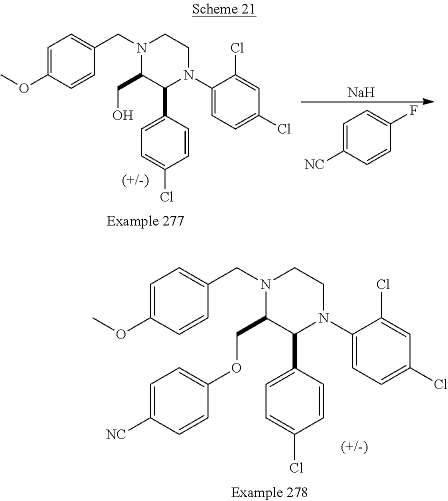

The N-benzyl piperazine Example 277 (160 mg) and NaH (46 mg, 60 wt % oil dispersion) were taken up in DMF. Then 4-fluoro-cyanobenzene (100 mg) was added, and the resulting solution was stirred at 25° C. (4 h). The reaction was partitioned between EtOAc and water. The aqueous layers were extracted with EtOAc. The combined organic layers were washed with brine and dried (MgSO₄). Filtration and concentration gave a yellow oil. Purification via thin-layer preparative chromatography (5/1 hexanes/EtOAc, SiO₂) furnished 4 mg (2%) of the ether Example 278 as an oil.

### Preparation of Examples 279-280

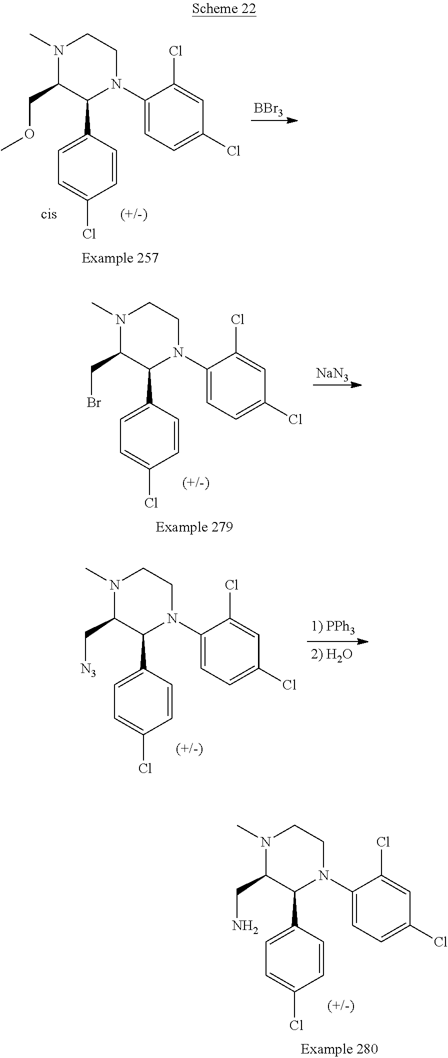

### Step 1:

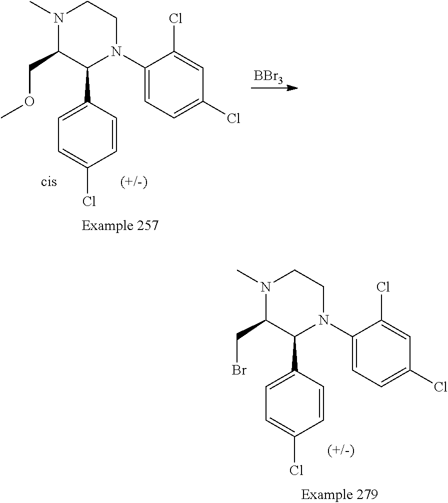

The piperazine Example 257 (1.3 g) was taken up in CH₂Cl₂. Boron tribromide (17 mL of a 1.0 M solution in CH₂Cl₂) was added, and the solution was stirred at 25° C. (16 h). The solution was diluted with CH₂Cl₂ and washed with saturated NaHCO_{3(aq)}. The aqueous layers were extracted with CH₂Cl₂. The combined organic layers were dried (MgSO₄), filtered, and concentrated. Purification via flash chromatography (CH₂Cl₂ then 10% EtOAc in CH₂Cl₂) furnished the bromide (1.1 g, 70%).

### Step 2:

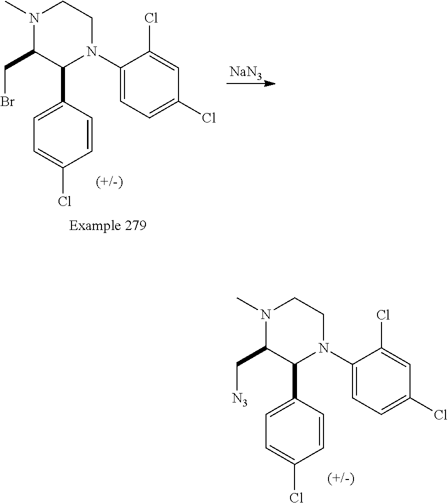 [SMILES: CN1CCN(c2ccc(Cl)cc2Cl)[C@@H](c2ccc(Cl)cc2)[C@H]1CBr ; CN1CCN(c2ccc(Cl)cc2Cl)[C@@H](c2ccc(Cl)cc2)[C@H]1CN=[N+]=[N-] ; "NaN3"]

The bromide (1.2 g) and NaN₃ (340 mg) were taken up in DMSO and stirred at 25° C. (72 h). The solution was partitioned between Et₂O and water. The aqueous layers were extracted with Et₂O. The combined organic layers were washed with brine, dried (MgSO₄), filtered, and concentrated to furnish the azide (1 g, 94%).

### Step 3:

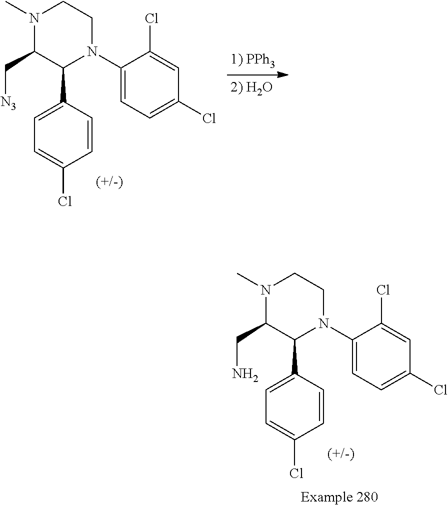 [SMILES: CN1CCN(c2ccc(Cl)cc2Cl)[C@@H](c2ccc(Cl)cc2)[C@H]1CN=[N+]=[N-] ; CN1CCN(c2ccc(Cl)cc2Cl)[C@@H](c2ccc(Cl)cc2)[C@H]1CN]

The azide (500 mg) and PPh₃ (640 mg) were taken up in THF (4 mL) and heated at reflux (65° C.) for 2 hours. Water (4 mL) was added, and the mixture was stirred at 40° C. (16 h). The mixture was concentrated. The residue was partitioned between EtOAc and water. The aqueous layer was extracted with EtOAc. The combined organic layers were washed with brine, dried (MgSO₄), filtered, and concentrated. Purification via thin-layer preparative chromatography (4% 7 N NH₃ in MeOH in CH₂Cl₂, SiO₂) furnished the amine (340 mg, 88%) Example 280.

### Preparation of Example 281-282

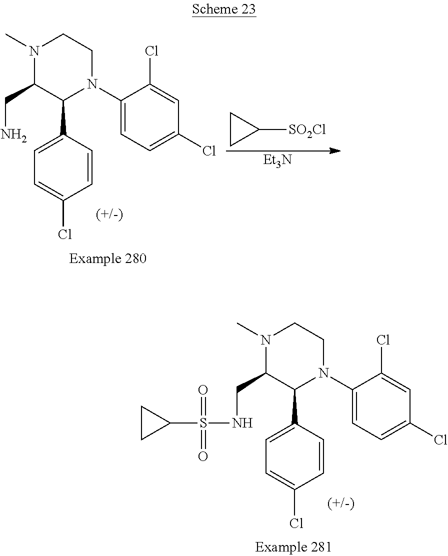

The amine Example 280 (160 mg) and Et₃N (95 mg) were taken up in CH₂Cl₂. Cyclopropylsulfonyl chloride (88 mg) was added, and the solution was stirred at 25° C. (16 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via thin-layer preparative chromatography (8% MeOH in CH₂Cl₂, SiO₂) furnished 138 mg (68%) of Example 281.

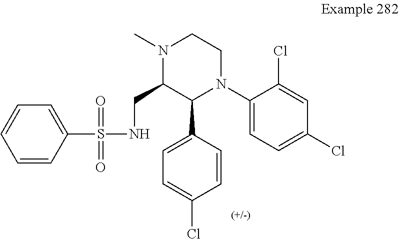 [SMILES: CN1CCN(c2ccc(Cl)cc2Cl)[C@@H](c2ccc(Cl)cc2)[C@H]1CNS(=O)(=O)c1ccccc1]

Example 282 was prepared according to the procedure described previously (Scheme 23) using benzenesulfonyl chloride.

### Preparation of Examples 283-288

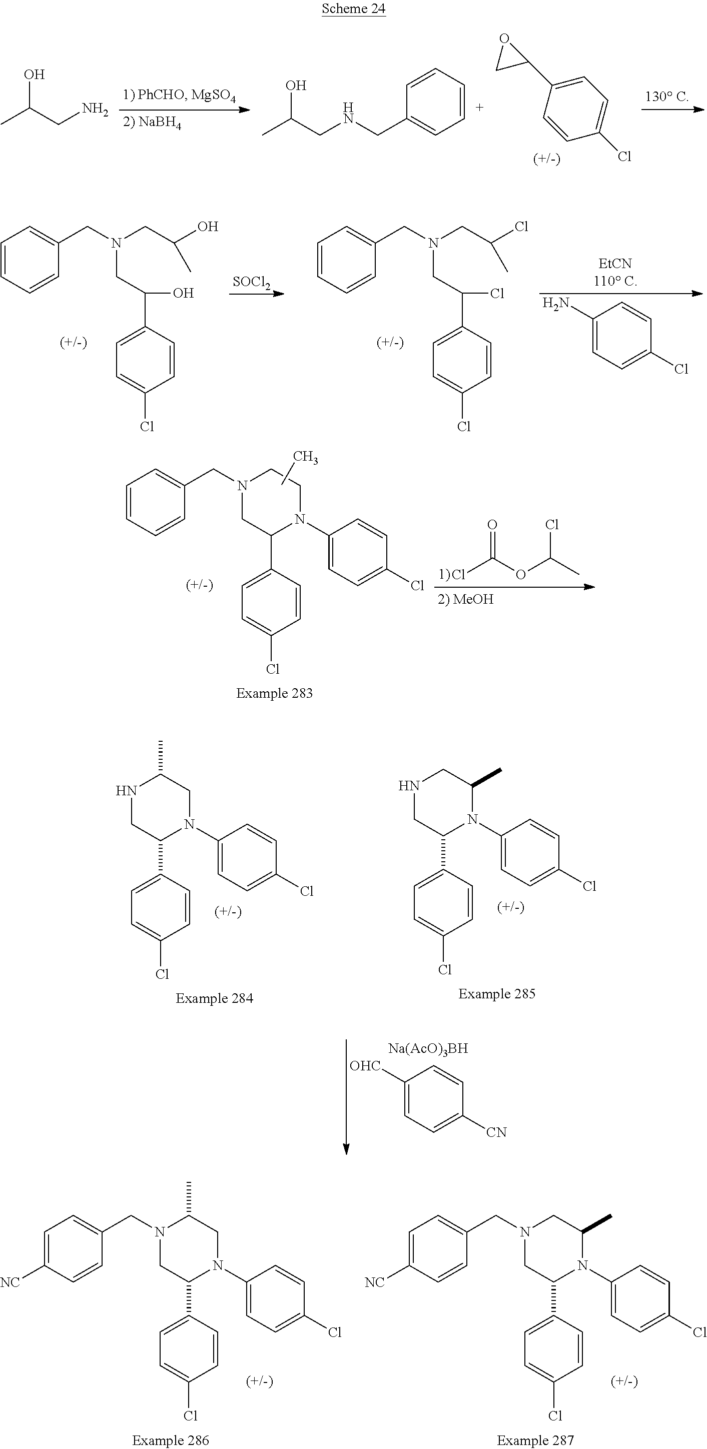

### Step 1:

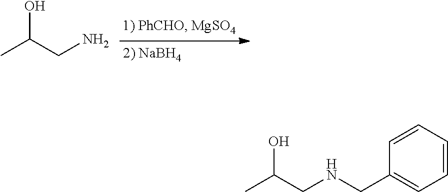

1-Amino-2-isopropanol (2 g), benzaldehyde (2.7 ml), and MgSO₄ (8 g) were taken up in CH₂Cl₂ and stirred at 25° C. (16 h). The mixture was filtered and concentrated which furnished an imine as a solid. The imine was taken up in MeOH and NaBH₄ (1 g) was added in portions. The solution was stirred at 25° C. (3 h). The solution was concentrated. The residue was partitioned between Et₂O and 1 M NCl_{(aq.)}. The aqueous acidic layer was extracted with Et₂O. The aqueous layer was cooled (0° C.) and rendered basic via addition of NaOH pellets (pH=11-12). The mixture was extracted with CH₂Cl₂. The combined organic layers were dried (MgSO₄), filtered, and concentrated which furnished the amino-alcohol as a thick oil (3.84 g, 87%).

### Step 2:

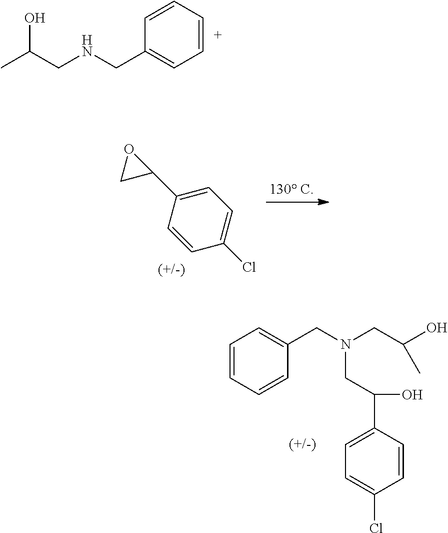 [SMILES: CC(O)CNCc1ccccc1 ; Clc1ccc(C2CO2)cc1 ; CC(O)CN(Cc1ccccc1)CC(O)c1ccc(Cl)cc1]

(+/−)-4-chloro-styrene oxide (2.8 mL) and the amino-alcohol (3.8 g) were heated neat at 130° C. (18 h) which furnished the diol as a thick gum. The diol was used in Step 3 without any further purification.

### Step 3:

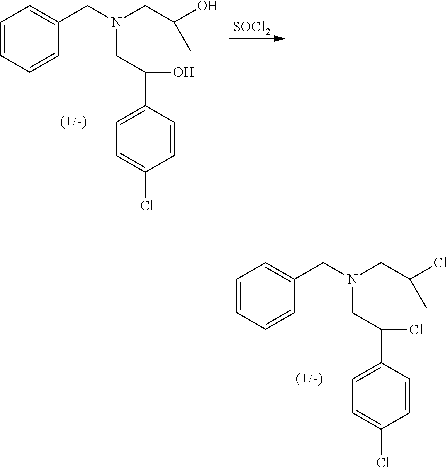

The crude diol from Step 2 (23 mmol) was taken up in DCE (i.e., dichloroethane). Thionyl chloride (4.3 mL) was added, and the solution was heated at reflux (85° C., 3.5 h). The solution was cooled and washed with 1 N NaOH_{(aq.)}. The aqueous layer was extracted with 0H₂Cl₂. The combined organic layers were dried (MgSO₄), filtered, and concentrated which furnished the dichloro-amine as a thick gum. This material was used without any further purification in Step 4.

### Step 4:

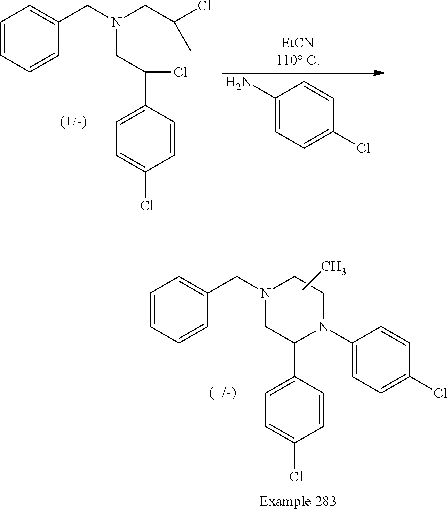

The dichloro-amine (7.9 g) and 4-chloroaniline (1.2 g) were taken up in EtCN and heated at 110° C. (19 h). The solution was concentrated and partitioned between CH₂Cl₂ and 1 N NaOH_{(aq.)}. The aqueous layer was extracted with CH₂Cl₂. The combined organic layers were dried (MgSO₄), filtered, and concentrated. Purification via flash chromatography (9/1 hexanes/Et₂O, SiO₂) gave 1.2 g (35%) of the piperazine Example 283 as a mixture of isomers.

### Step 5:

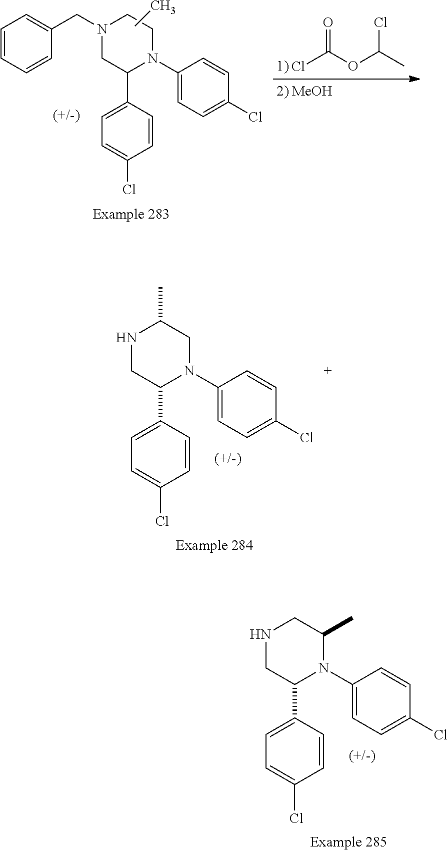

The piperazine Example 283 (1 g) and 1-chloroethyl-chloroformate ((0.3 mL) were taken up in DCE and heated (85° C., 18 h). The solution was concentrated. The residue was taken up in MeOH and heated at reflux (80° C., 3.5 h). The solution was concentrated. The residue was partitioned between CH₂Cl₂ and 1 N NaOH_{(aq.)}. The aqueous layer was extracted with CH₂Cl₂. The combined organic layers were dried (MgSO₄). Filtration and concentration gave a yellow oil. Purification via thin-layer preparative chromatography (9/1 CH₂Cl₂/MeOH, SiO₂) gave 100 mg of the 2,5-cis isomer Example 284 and 50 mg of the 2,6-trans isomer Example 285.

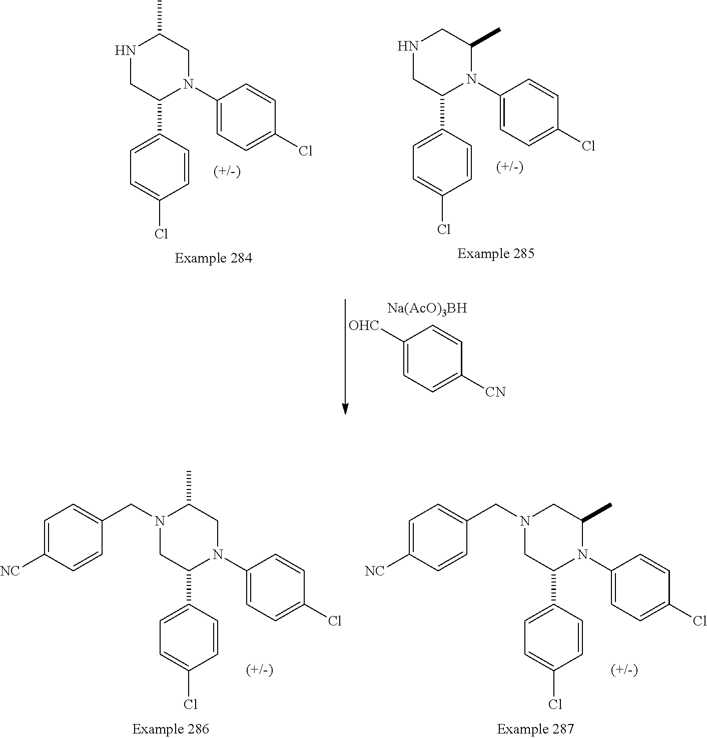

### Step 6:

The 2,5-cis-NH piperazine Example 284 (100 mg), 4-cyanobenzaldehyde (48 mg), and Na(AcO)₃BH (127 mg) were taken up in CH₂Cl₂ and stirred at 25° C. (19 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via thin-layer preparative chromatography (4/1 hexanes/EtOAc, SiO₂) gave 49 mg (37%) of Example 286 as a colorless oil. Following the same procedure, the 2,6-trans-NH piperazine Example 285 was converted into Example 287.

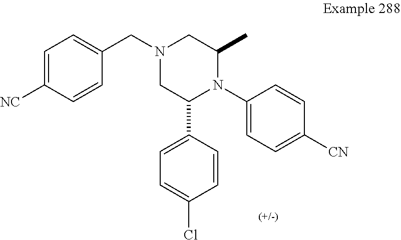

Example 288 was prepared according to conditions described for Example 287 using 4-cyano-aniline in Step 4 of Scheme 24.

### Preparation of Examples 289-294

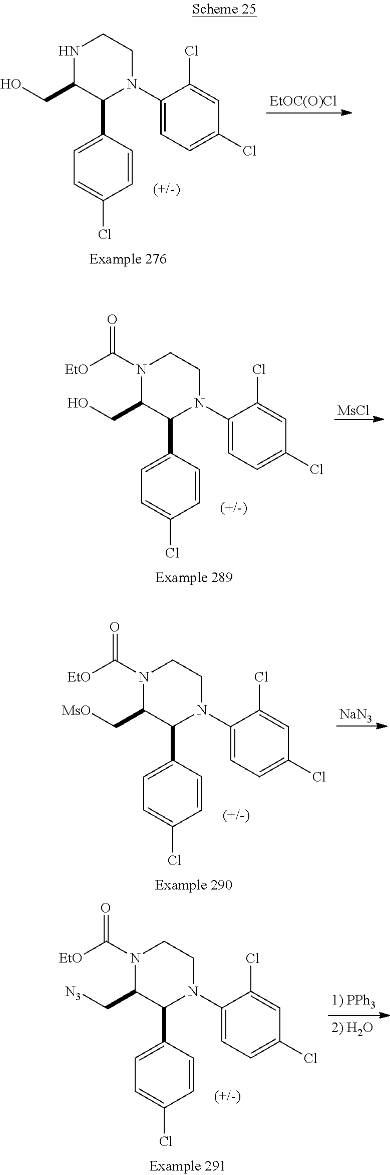

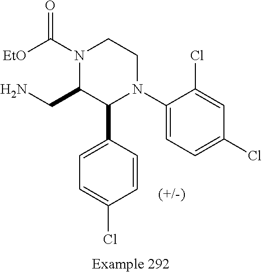 [SMILES: CCOC(=O)N1CCN(c2ccc(Cl)cc2Cl)[C@@H](c2ccc(Cl)cc2)[C@H]1CN]

### Step 1:

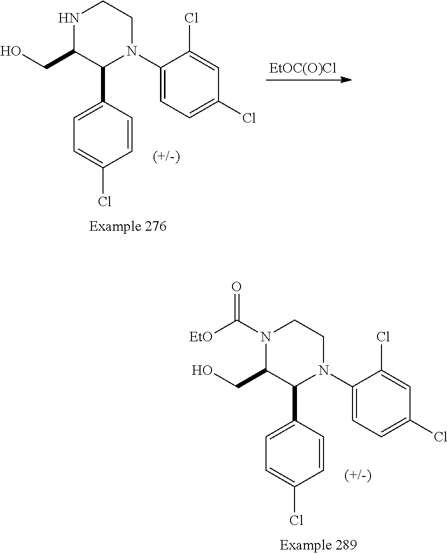 [SMILES: "EtOC(O)Cl" ; OC[C@H]1NCCN(c2ccc(Cl)cc2Cl)[C@H]1c1ccc(Cl)cc1 ; CCOC(=O)N1CCN(c2ccc(Cl)cc2Cl)[C@@H](c2ccc(Cl)cc2)[C@H]1CO]

The amino-alcohol Example 276 from Scheme 20 (400 mg) and iPr₂NEt (170 mg) were taken up in CH₂Cl₂. Ethyl chloroformate (130 mg) was added, and the solution was stirred at 25° C. for 30 minutes. The solution was diluted with CH₂Cl₂ and washed with saturated NaHCO_{3(aq.)}. The aqueous layer was extracted with CH₂Cl₂. The combined organic layers were dried (MgSO₄), filtered, and concentrated. Purification via thin-layer preparative chromatography (15% EtOAc in CH₂Cl₂, SiO₂) furnished the carbamate Example 289.

### Step 2:

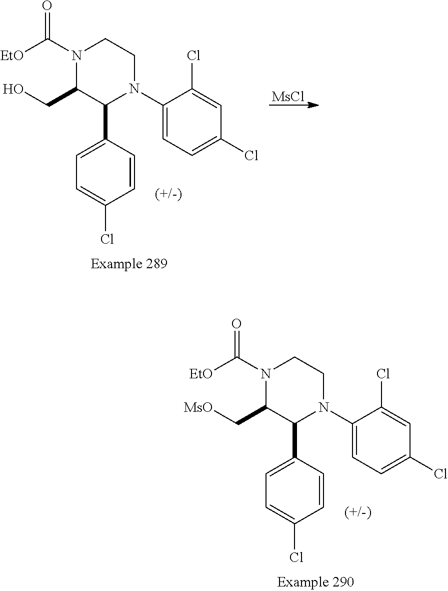 [SMILES: "MsCl" ; CCOC(=O)N1CCN(c2ccc(Cl)cc2Cl)[C@@H](c2ccc(Cl)cc2)[C@H]1CO ; CCOC(=O)N1CCN(c2ccc(Cl)cc2Cl)[C@@H](c2ccc(Cl)cc2)[C@H]1COS(C)(=O)=O]

The carbamate Example 289 and Et₃N (4 eq.) were taken up in CH₂Cl₂. Methanesulfonyl chloride (4 eq.) was added at 25° C. The solution was stirred at 25° C. for 15 minutes. The solution was diluted with CH₂Cl₂ and washed with saturated NaHCO_{3(aq.)}. The aqueous layer was extracted with CH₂Cl₂. The combined organic layers were dried (MgSO₄). Filtration and concentration gave the mesylate Example 290, which was used without further purification in Step 3.

### Step 3:

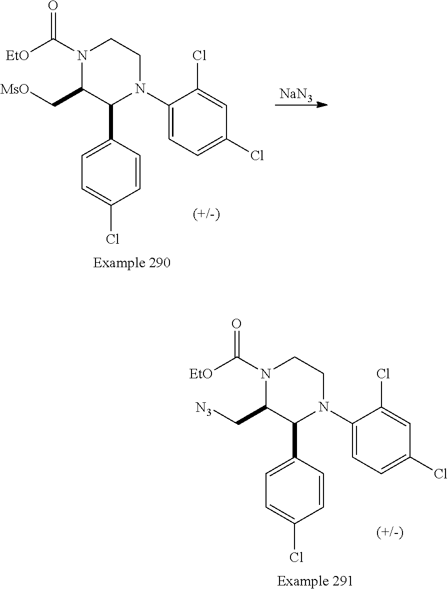

The mesylate Example 290 from above and sodium azide (130 mg) were taken up in acetone and heated at reflux (60° C., 18 h). More sodium azide was added (500 mg), and the reaction was heated for an additional 18 h (60° C.). The solution was concentrated, and the residue was partitioned between EtOAc and water. The aqueous layer was extracted with EtOAc. The combined organic layers were washed with brine and dried (MgSO₄). Filtration and concentration gave the azide Example 291 as a yellow oil (320 mg. 64% from the amino-alcohol).

### Step 4:

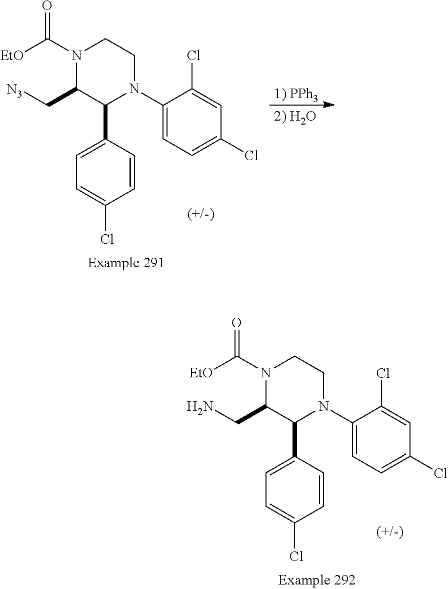

The azide Example 291 (320 mg) and PPh₃ (220 mg) were taken up in THF and heated at 65° C. (5 h). Water (2 mL) was added, and the solution was heated at reflux (65° C., 18 h). The solution was concentrated, and the residue was partitioned between EtOAc and water. The aqueous layer was extracted with EtOAc. The combined organic layers were washed with brine and dried (MgSO₄). Filtration and concentration gave the crude amine. Purification via thin-layer preparative chromatography (10% MeOH in CH₂Cl₂, SiO₂) gave 210 mg (70%) of the amine Example 292.

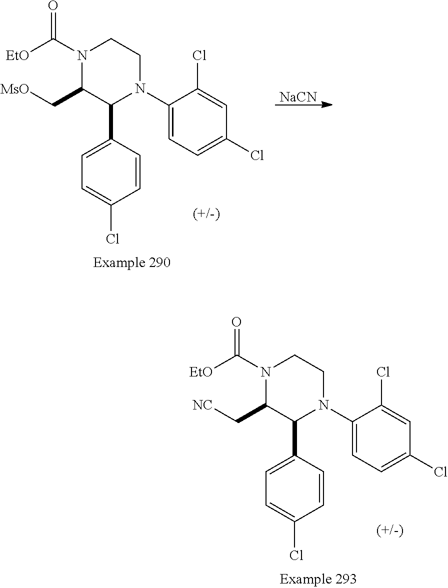

The mesylate Example 290 from Scheme 25 (300 mg) and NaCN (43 mg) were taken up in DMF and heated at 90° C. (18 h). The solution was partitioned between EtOAc and water. The aqueous layer was extracted with EtOAc. The combined organic layers were washed with brine and dried (MgSO₄). Filtration and concentration gave a yellow oil. Purification via thin-layer preparative chromatography (5% EtOAc in CH₂Cl₂, SiO₂) gave the cyano-carbamate Example 293 (130 mg, 50%) as a white solid.

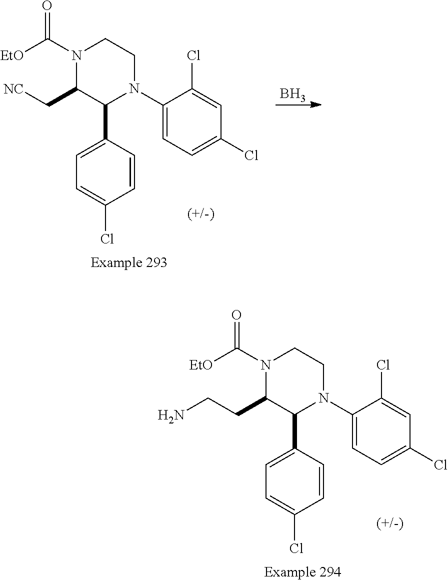

The cyano-carbamate Example 293 (120 mg) was taken up in THF. Borane-THF (0.5 mL of a 1.0 M solution in THF) was added, and the solution was heated to reflux (70° C., 5 h). Additional BH₃ (2.2 mL of a 1.0 M solution in THF) was added to the reaction, and the reaction was heated at reflux (70° C., 18 h). The solution was cooled and quenched with 1 M HCl_{(aq.)}. The solution was stirred at 25° C. (20 minutes) and then rendered basic with 1 N NaOH_{(aq.)}. The mixture was extracted with EtOAc. The combined organic layers were washed with brine and dried (MgSO₄). Filtration and concentration gave a yellow oil. Purification via thin-layer preparative chromatography (15% MeOH in CH₂Cl₂, SiO₂) gave 100 mg (83%) of the amine as a yellow oil.

### Preparation of Examples 295-301

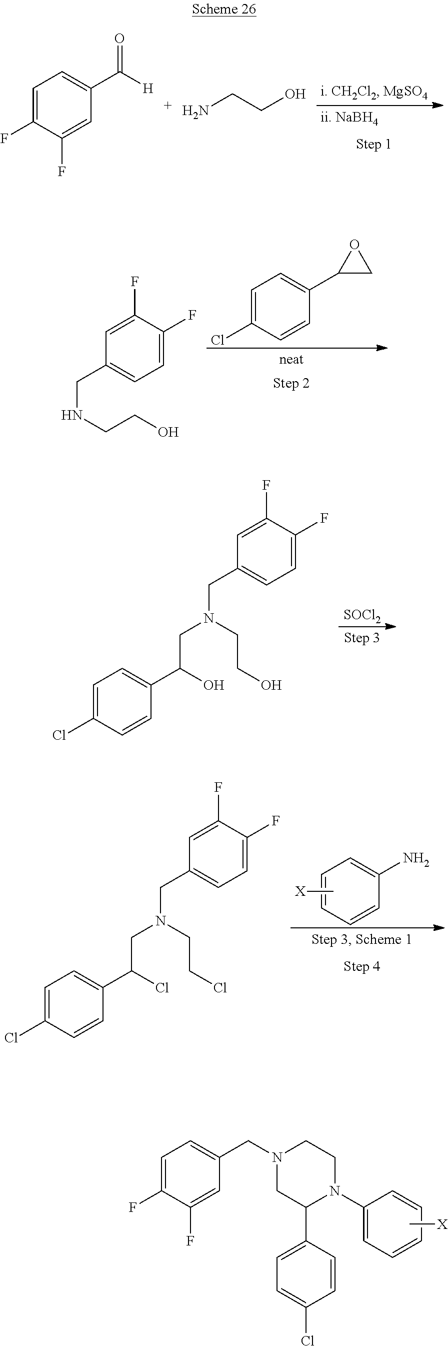

### Step 1:

To 3,4-difluorobenzaldehyde (15.0 g, 105 mmol) in CH₂Cl₂ (100 mL) was added ethanolamine (6.4 g, 105 mmol) and MgSO₄ (32 g). The reaction mixture was stirred for 20 h at room temperature. The reaction was filtered and concentrated in vacuo. The residue was taken up into MeOH (100 mL), cooled to 0° C., and NaBH₄ was added. The reaction mixture was stirred for 2 h allowing the cold bath to warm to room temperature. The reaction was then concentrated in vacuo and 3N HCl was added. The mixture was extracted with ether. The aqueous layer was then rendered basic with 3N NaOH and then extracted with EtOAc. The EtOAc extractions were combined and washed with water and brine. The organic layer was dried (MgSO₄), filtered, and concentrated in vacuo to provide the corresponding amino-alcohol (14.0 g, 75 mmol).

### Step 2:

To the amino-alcohol from step 1 (7.0 g, 37 mmol) was added 4-chlorostyrene oxide (5.0 mL, 41.5 mmol). The neat reaction mixture was warmed to 130° C. and stirred for 20 h, cooled to room temperature and purified by silica gel chromatography (3-5% MeOH/CH₂Cl₂) to provide the corresponding amino-diol (12.6 g, 36.8 mmol).

### Step 3:

To the amino-diol prepared in step 2 (12.6 g, 37 mmol) in CHCl₃ (122 mL) at 0° C. was added SOCl₂ (61 mL) dropwise. After addition, the reaction mixture was warmed to reflux and stirred for 2 h. The reaction was concentrated in vacuo. The residue was taken up into CH₂Cl₂ and stirred vigorously with saturated NaHCO₃. The organic layer was washed with brine and dried (MgSO₄). The organic layer was filtered and concentrated in vacuo. The residue was purified by silica gel chromatography (10% EtOAc/hexane) to provide the corresponding amino-dichloride (10.0 g, 26 mmol).

### Step 4:

Following the procedure of step 3 in Scheme 1, the anilines listed in Table XI were used in place of 2,4-dichloroaniline to provide the desired diarylpiperazine compound.

TABLE XI

| Example # | Example Structure | aniline |
|---|---|---|
| 295 | 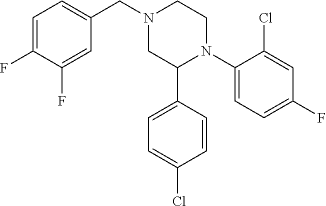 [SMILES: Fc1ccc(N2CCN(Cc3ccc(F)c(F)c3)CC2c2ccc(Cl)cc2)c(Cl)c1] | 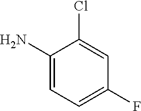 |
| 296 | 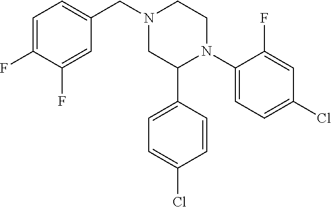 [SMILES: Fc1ccc(CN2CCN(c3ccc(Cl)cc3F)C(c3ccc(Cl)cc3)C2)cc1F] | 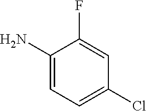 |
| 297 | 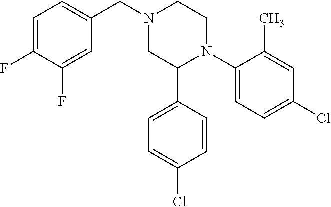 [SMILES: Cc1cc(Cl)ccc1N1CCN(Cc2ccc(F)c(F)c2)CC1c1ccc(Cl)cc1] | 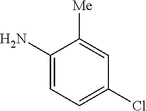 |
| 298 | 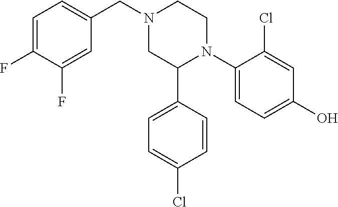 [SMILES: Oc1ccc(N2CCN(Cc3ccc(F)c(F)c3)CC2c2ccc(Cl)cc2)c(Cl)c1] | 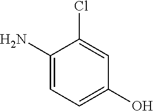 |
| 299 | 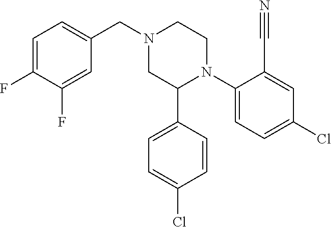 [SMILES: N#Cc1cc(Cl)ccc1N1CCN(Cc2ccc(F)c(F)c2)CC1c1ccc(Cl)cc1] | 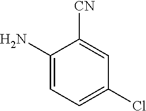 |
| 300 | 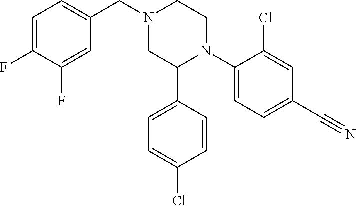 [SMILES: N#Cc1ccc(N2CCN(Cc3ccc(F)c(F)c3)CC2c2ccc(Cl)cc2)c(Cl)c1] | 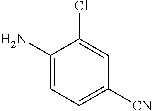 |
| 301 | 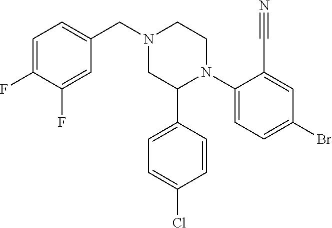 [SMILES: N#Cc1cc(Br)ccc1N1CCN(Cc2ccc(F)c(F)c2)CC1c1ccc(Cl)cc1] | 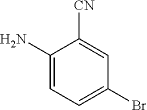 |

### Preparation of Example 302

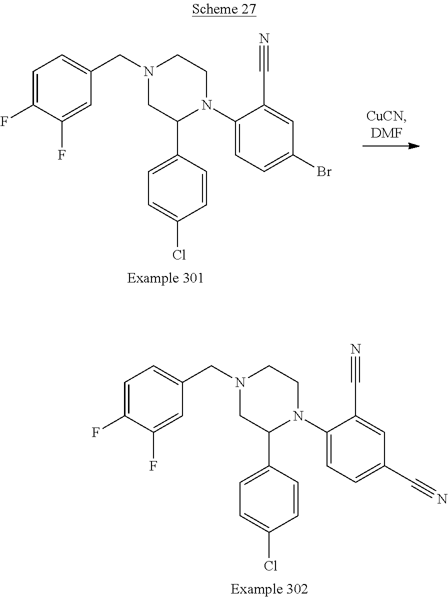 [SMILES: N#Cc1cc(Br)ccc1N1CCN(Cc2ccc(F)c(F)c2)CC1c1ccc(Cl)cc1 ; N#Cc1ccc(N2CCN(Cc3ccc(F)c(F)c3)CC2c2ccc(Cl)cc2)c(C#N)c1]

Example 302 was prepared using the procedure of step 1, Scheme 29 used to prepare Example 308.

### Preparation of Examples 303-304

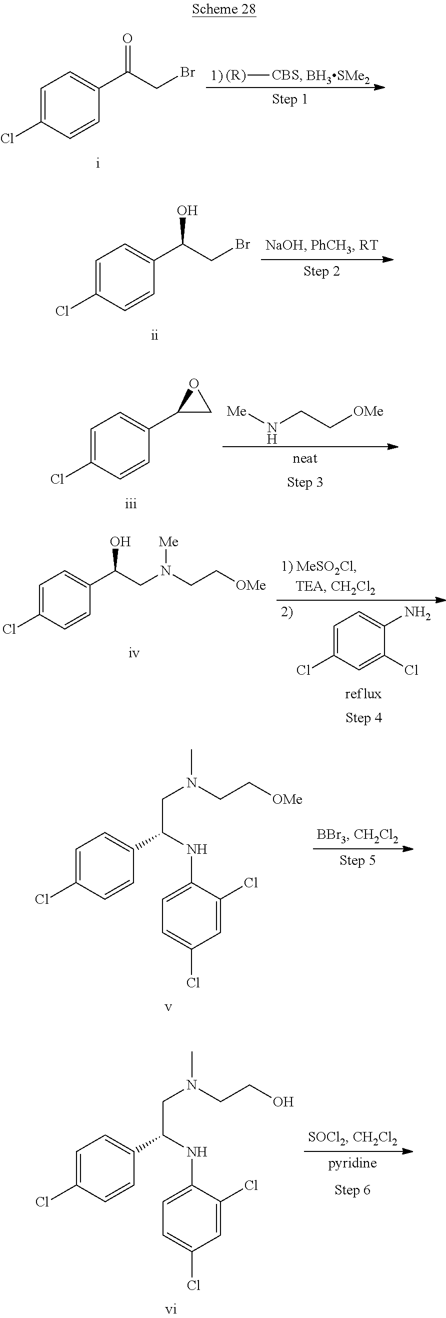

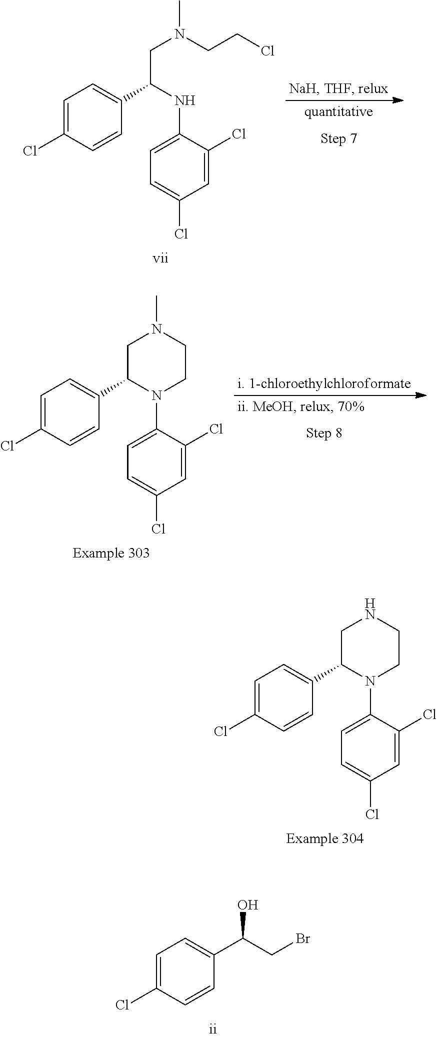 [SMILES: CN(CCCl)C[C@H](Nc1ccc(Cl)cc1Cl)c1ccc(Cl)cc1 ; CN1CCN(c2ccc(Cl)cc2Cl)[C@H](c2ccc(Cl)cc2)C1 ; Clc1ccc([C@@H]2CNCCN2c2ccc(Cl)cc2Cl)cc1 ; O[C@@H](CBr)c1ccc(Cl)cc1]

### Step 1:

To 2-bromo-4′-chloroacetophenone (233 g, 1000 mmol) in THF (1 L) at 0° C. was added (R)-2-methyl-CBS-oxazaborolidine (available from Aldrich) (1.0 M in THF, 200 mL, 200 mmol) through an addition funnel. BH₃.SMe₂ (2.0 M in THF, 300 mL, 600 mL) was added slowly over 25 min. The reaction was stirred at room temperature for 2 h. The reaction was cooled to 0° C., and MeOH (200 mL) was added slowly (with gas evolution). The resulting solution was concentrated in vacuo and then diluted with CH₂Cl₂ (3.5 L). The organic layer was washed with 1N HCl, water, and brine, then dried (MgSO₄), filtered, and concentrated in vacuo to provide bromo-alcohol ii as an oil that solidified on standing (237 g).

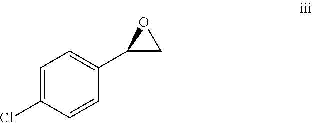

### Step 2:

The bromo-alcohol ii from step 2 (237 g, 1000 mmol) was dissolved in toluene (3.5 L) and 3N NaOH (3.5 L) was added. The reaction was stirred vigorously at room temperature for 3 h. The organic layer was washed with water and brine and dried (MgSO₄), then filtered and concentrated in vacuo to provide the epoxide iii (154 g, 1000 mmol). The ee (i.e., enantiomeric excess) of the epoxide was found to be ≧96% ee by HPLC [R,R-Whelko-O-1, 99.75:0.25 hexane/IPA, 1 mL/min, 220 nm. Isomer A retention time 10.5 min, isomer B (major) 14.1 min)].

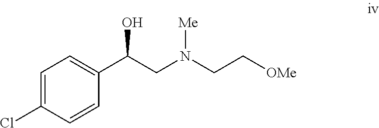 [SMILES: COCCN(C)C[C@H](O)c1ccc(Cl)cc1]

### Step 3:

To the epoxide iii prepared in step 2 (102 g, 662 mmol) was added N-(2-methoxyethyl)methyl amine (83 g, 930 mmol). The reaction mixture was heated neat (i.e., without solvent) to 100° C. and stirred for 18 h. The reaction mixture was cooled to room temperature and then concentrated in vacuo to remove the excess amine, thereby providing amino-alcohol iv as a mixture of regiosomeric ring opening products (−12:1) (154 g, 96%).

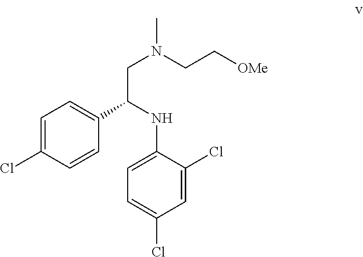 [SMILES: COCCN(C)C[C@H](Nc1ccc(Cl)cc1Cl)c1ccc(Cl)cc1]

### Step 4:

To the amino-alcohol iv prepared in step 3 (101 g, 416 mmol) in CH₂Cl₂ (2 L) at 0° C. was added TEA (i.e., triethylamine) (145 mL, 1040 mmol) followed by methanesulfonyl chloride (52.4 g, 460 mmol). The reaction mixture was stirred at room temperature for 2 h and then methanesulfonyl chloride (4 mL, 6 mmol) was added. The reaction mixture was stirred for an additional 1 h, then 2,4-dichloroaniline (67.5 g, 416 mmol) was added and the mixture was warmed to reflux. The refluxing mixture was stirred for 20 h and cooled to room temperature. CH₂Cl₂ (2 L) was added, and the reaction mixture was washed with saturated NaHCO₃, water, and brine, then dried (Mg₂SO₄), filtered, and concentrated in vacuo to provide v (167 g) as a pale oil that was used directly in the following synthetic step. This process is expected to go with retention of configuration as described in Tetrahedron: Asymmetry 10 (1999) 2655-2663 (herein incorporated by reference).

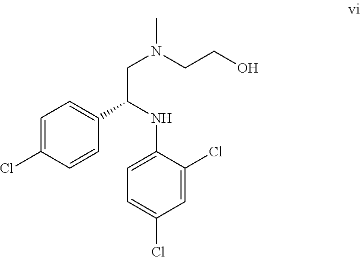 [SMILES: CN(CCO)C[C@H](Nc1ccc(Cl)cc1Cl)c1ccc(Cl)cc1]

### Step 5:

To v (167 g, 433 mmol) in CH₂Cl₂ (1.5 L) at 0° C. was added BBr₃ (164 g, 433 mmol) over 30 minutes. The reaction was stirred at 0° C. for 1 h and then for additional 3.5 h at room temperature. Saturated NaHCO₃ (3.5 L) was added slowly with gas evolution from the reaction mixture. The reaction mixture was extracted with CH₂Cl₂. The organic extracts were combined and washed with water (2 L), and brine (2 L), dried (MgSO₄), filtered, and concentrated in vacuo to provide a brown oil that was purified by silica gel chromatography (30% EtOAc/hexane) to yield amino-alcohol vi (90 g, 241 mmol).

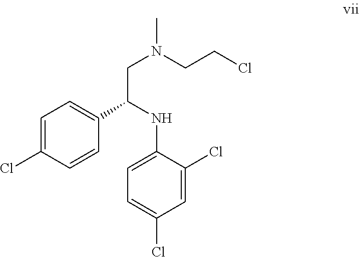 [SMILES: CN(CCCl)C[C@H](Nc1ccc(Cl)cc1Cl)c1ccc(Cl)cc1]

### Step 6:

To the amino-alcohol vi prepared in step 5(90 g, 240 mmol) in CH₂Cl₂ (1 L) at 0° C. was added pyridine (40 mL, 480 mmol) followed by thionyl chloride (53 mL, 720 mmol). The cold bath was removed and the reaction was stirred at room temperature for 4 h and then concentrated in vacuo without heating. The sample was taken up into EtOAc, (2 L) and cooled to 0° C. Saturated NaHCO₃ (1 L) was cautiously added (with gas evolution). The EtOAc layer was washed with water (1 L), brine (1 L), dried (MgSO₄/NaSO₄), filtered and concentrated in vacuo to provide a brown oil that was purified by silica gel chromatography (10% EtOAc/hexane) to give chloro-amine vii as a pale brown oil (84 g, 89%).

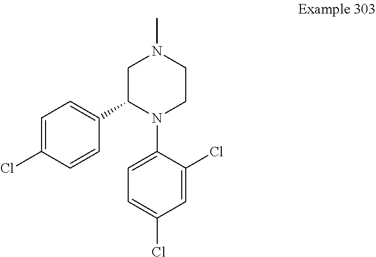 [SMILES: CN1CCN(c2ccc(Cl)cc2Cl)[C@H](c2ccc(Cl)cc2)C1]

### Step 7:

To chloro-amine vii prepared in step 6 (84 g, 214 mmol) in THF (1 L) was added NaH (60% dispersion in mineral oil) (21.14 g, 535 mmol) in one portion. The reaction mixture was warmed to reflux and stirred for 4 h, then cooled to 0° C. 1 L of an ice/water mixture was then added (gas evolution). CH₂Cl₂ (2 L) was added and the reaction mixture was stirred. The aqueous phase was washed with CH₂Cl₂, and the CH₂Cl₂ layers were then combined and washed with water (1 L), and brine (1 L). The reaction mixture was dried (MgSO₄), filtered, and concentrated in vacuo to provide Example 303 as a brown oil (83 g) contaminated with mineral oil. The material was used in the following step directly, without further purification.

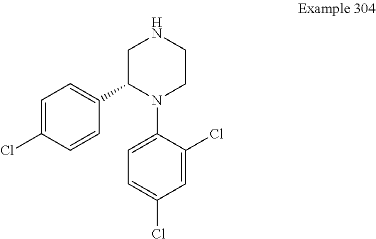 [SMILES: Clc1ccc([C@@H]2CNCCN2c2ccc(Cl)cc2Cl)cc1]

### Step 8:

To N-methylpiperazine Example 303 (83 g) in DCE (0.8 L) at room temperature was added proton sponge (9.2 g, 43 mmol) followed by 1-chloroethylchloroformate (46.3 mL, 429 mmol). The reaction was warmed to reflux and stirred for 3 h. The reaction mixture was cooled to room temperature and concentrated in vacuo. MeOH (1 L) was added and the reaction mixture was warmed to reflux. The reaction mixture was stirred at reflux for 1.5 h and cooled to room temperature, then concentrated in vacuo. CH₂Cl₂ (1.5 L) was added and the reaction mixture was washed with saturated NaHCO₃, water, and brine. The reaction mixture was then dried (MgSO₄), filtered, and concentrated in vacuo. The concentrated residue was purified by silica gel chromatography (CH₂Cl₂ then 5% MeOH/CH₂Cl₂) to provide the piperazine Example 304 (60 g, 214 mmol). The ee was determined to be 98% ee by HPLC analysis (Chiralcel OD column, 94:6 hexane/isopropyl alcohol, 1 mL/min, 254 nm-isomer A retention time 8.4 min, isomer B 10.9 min).

### Preparation of Example 305

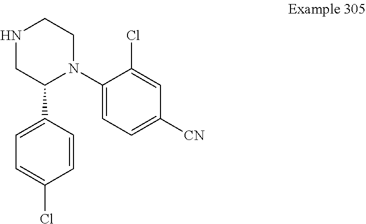 [SMILES: N#Cc1ccc(N2CCNC[C@H]2c2ccc(Cl)cc2)c(Cl)c1]

The piperazine Example 305 was prepared using a procedure similar to the procedure used to prepare Example 304, except that 4-amino-3-chlorobenzonitrile was used in place of 2,4-dichloroaniline in Step 4.

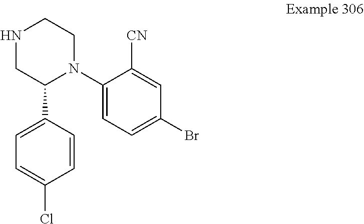 [SMILES: N#Cc1cc(Br)ccc1N1CCNC[C@H]1c1ccc(Cl)cc1]

### Preparation of Example 306

The piperazine Example 306 was prepared using a procedure similar to the procedure used to prepare Example 304 except that 2-amino-5-bromobenzonitrile was used in place of 2,4-dichloroaniline in Step 4.

### Preparation of Examples 307-309

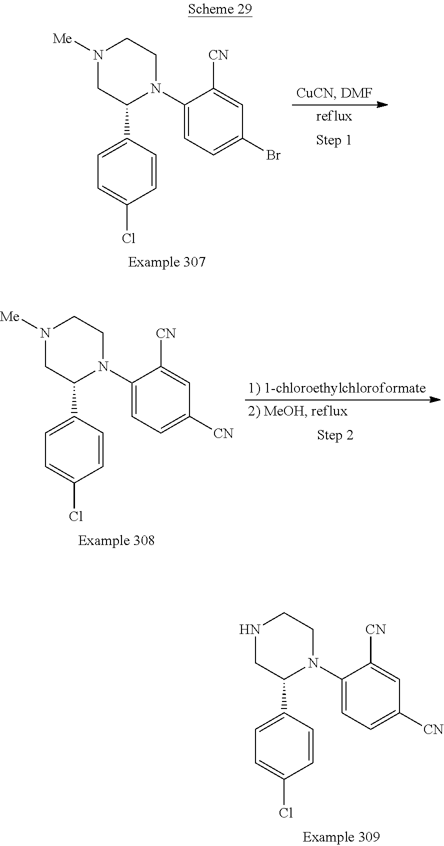 [SMILES: CN1CCN(c2ccc(Br)cc2C#N)[C@H](c2ccc(Cl)cc2)C1 ; CN1CCN(c2ccc(C#N)cc2C#N)[C@H](c2ccc(Cl)cc2)C1 ; N#Cc1ccc(N2CCNC[C@H]2c2ccc(Cl)cc2)c(C#N)c1]

### Step 1:

The N-methylpiperazine Example 307 was prepared using a procedure similar to the procedure used to prepare Example 303 except that 2-amino-5-bromobenzonitrile was used in place of 2,4-dichloroaniline in Step 4. The N-methylpiperazine Example 307 (2.70 g, 7 mmol) in DMF (14 mL) was treated with CuCN (1.88 g, 21 mmol). The reaction mixture was warmed to reflux and stirred for 48 h. The reaction mixture was cooled to room temperature and EtOAc was added followed by saturated NH₄Cl/NH₄OH 9:1 solution. The mixture was stirred vigorously for 15 minutes and then extracted with EtOAc. The organic layers were combined and washed with water, and brine. The organic layer (MgSO₄) was dried, filtered, and concentrated in vacuo. The residue was then purified by silica gel chromatograpy (4% MeOH/CH₂Cl₂) to provide the N-methylpiperazine Example 308 (1.0 g, 3.0 mmol).

### Step 2:

The N-methylpiperazine Example 308 was demethylated to form Example 309 as in Step 8 of Scheme 28.

### Preparation of Example 310

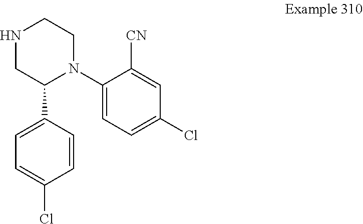 [SMILES: N#Cc1cc(Cl)ccc1N1CCNC[C@H]1c1ccc(Cl)cc1]

The 4-chloro-2-cyanopiperazine Example 310 was prepared using a procedure similar to the procedure used to prepare Example 304 of Scheme 28 except that 2-amino-5-chlorobenzonitrile was used in place of 2,4-dichloroaniline in step 4.

### Preparation of Example 311

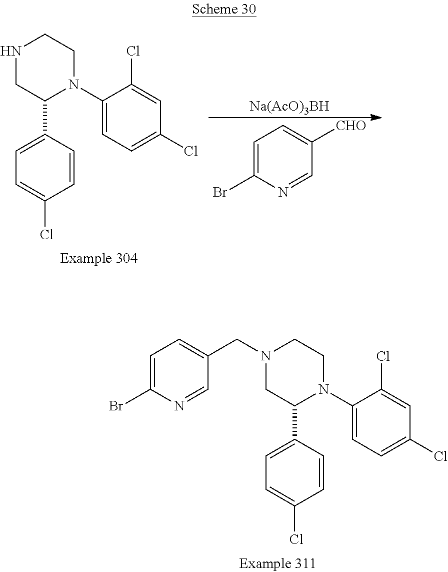

The enantio-enriched piperazine Example 304 (500 mg), 6-bromo-pyridine-3-carbaldehyde (326 mg), and Na(AcO)₃BH (371 mg) were taken up in CH₂Cl₂ and stirred at 25° C. (18 h). The reaction mixture was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via flash chromatography (4/1 hexanes/EtOAc, SiO₂) gave 376 mg (50%) of the bromo-pyridine Example 311 as an oil.

### Preparation of Examples 312-313

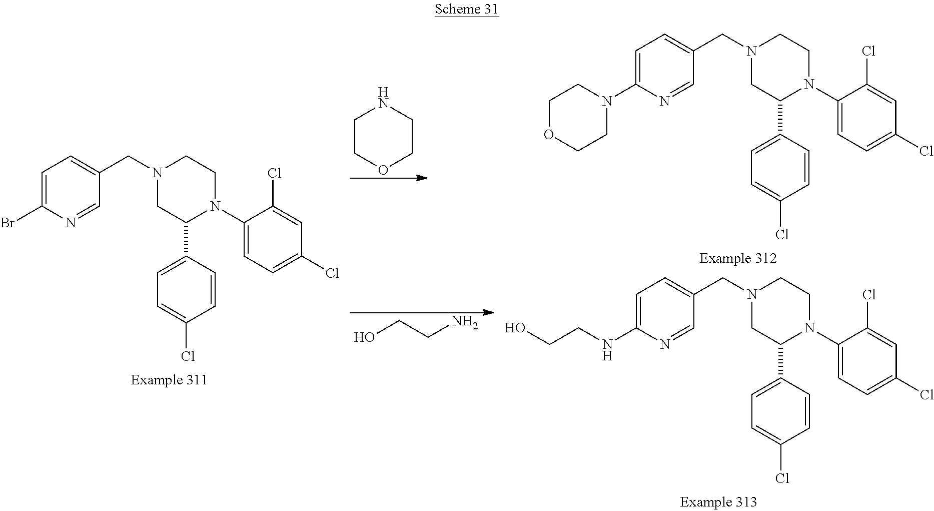

The bromo-pyridine Example 311 (80 mg) and morpholine (0.3 mL) were heated at 100° C. (18 h). The solution was cooled and partitioned between CH₂Cl₂ and 1 N NaOH_{(aq.)}. The aqueous solution was extracted with CH₂Cl₂. The combined organic layers were dried (MgSO₄), filtered, and concentrated. Purification via thin-layer preparative chromatography (1/1 hexanes/EtOAc, SiO₂) gave 54 mg (65%) of Example 312 as a colorless oil.

In a similar manner, the reaction of Example 311 with ethanol-amine furnished Example 313 as a colorless oil.

### Preparation of Examples 314 and 315

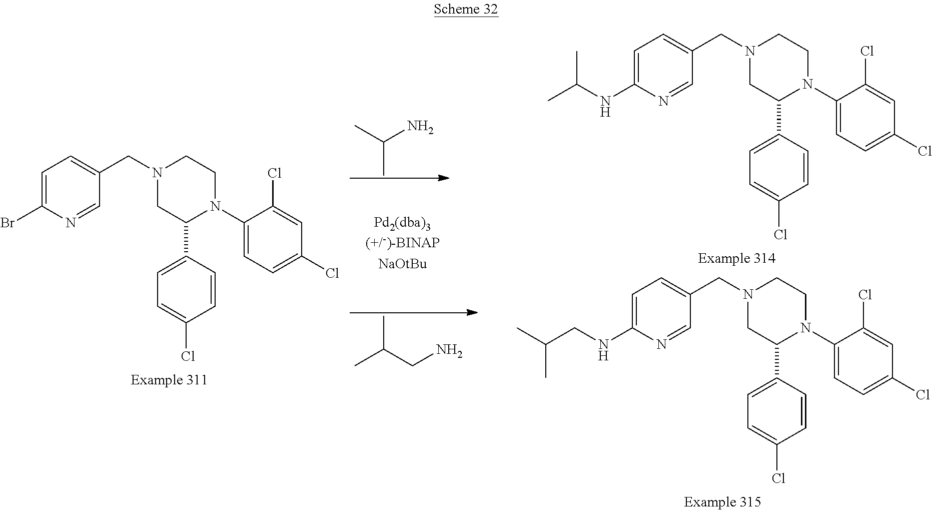

The bromo-pyridine (85 mg) Example 311, racemic-BINAP (i.e., 2,2′-bis-diphenylphosphanyl-[1,1′]binaphthalenyl; 40 mg), Pd₂(dba)₃ (15 mg), and NaOtBu (100 mg) were taken up in iso-propyl amine and heated at 100° C. in a sealed tube (18 h). The solution was diluted with Et₂O and filtered through Celite. Concentration gave the crude product. Purification via thin-layer preparative chromatography (2/1 hexanes/EtOAc, SiO₂) gave 40 mg (48%) of Example 314 as an oil.

In a similar manner, the reaction of Example 311 and iso-butyl amine furnished Example 315 as an oil.

### Preparation of Example 316

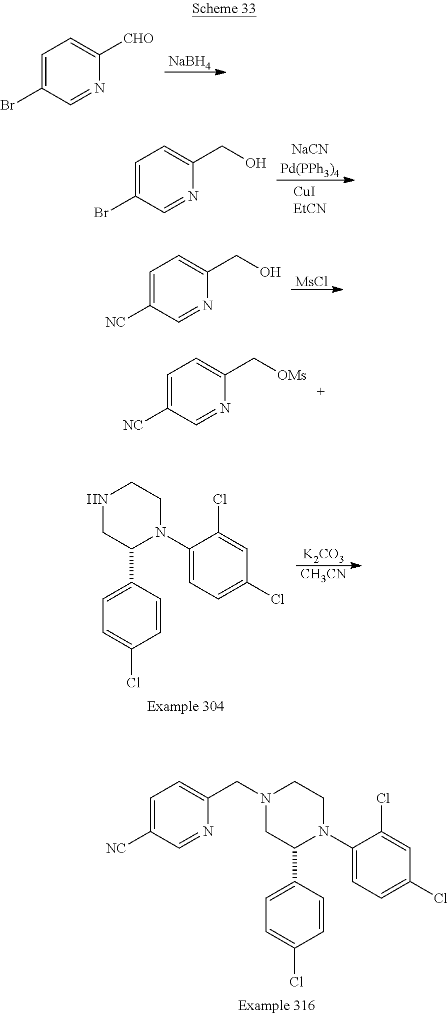

### Step 1:

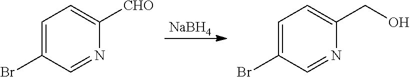 [SMILES: O=Cc1ccc(Br)cn1 ; "NaBH4" ; OCc1ccc(Br)cn1]

The 5-bromo-pyridine-2-carbaldehyde (2.0 g) was taken up in MeOH and cooled to 0° C. Sodium borohydride (450 mg) was added in portions at 0° C. The solution was warmed to 25° C. and stirred at that temperature for 1.5 h. The solution was concentrated, and the residue was quenched with 1 M HCl_{(aq.)}. The solution was stirred at 25° C. for 0.5 h. The solution was rendered basic via addition of solid K₂CO₃. The mixture was extracted with CH₂Cl₂. The combined organic layers were dried (MgSO₄), filtered, and concentrated to give (5-bromo-pyridin-2-yl)-methanol as a white solid.

### Step 2:

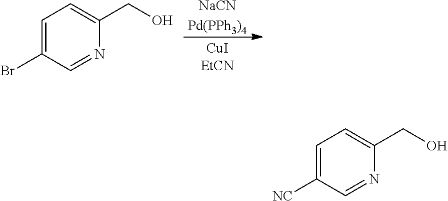 [SMILES: OCc1ccc(Br)cn1 ; N#Cc1ccc(CO)nc1 ; "NaCNPd(PPh3)4"]

(5-Bromo-pyridin-2-yl)-methanol (1.0 g), NaCN (521 mg), Pd(PPh₃)₄ (612 mg), and CuI (200 mg) were taken up in degassed EtCN and heated at 110° C. (4 h). The solution was partitioned between EtOAc and 10% NH₄OH_{(aq.)}. The aqueous layer was extracted with EtOAc. The combined organic layers were washed with brine and dried (MgSO₄). Filtration and concentration gave a yellow solid. Purification via flash chromatography (½ hexanes/EtOAc, SiO₂) gave 341 mg (48%) of (5-cyano-pyridin-2-yl)-methanol as a white solid.

### Step 3:

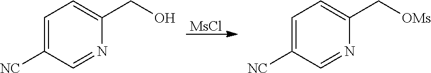 [SMILES: "MsCl" ; N#Cc1ccc(CO)nc1 ; CS(=O)(=O)OCc1ccc(C#N)cn1]

(5-Cyano-pyridin-2-yl)-methanol (100 mg) and Et₃N (0.14 m) were taken up in CH₂Cl₂ and cooled to 0° C. Methansulfonyl chloride (0.1 mL) was added and the solution was stirred at 0° C. for 2 h. The solution was diluted with CH₂Cl₂ and washed with saturated NaHCO₃ (aq.). The aqueous layer was extracted with CH₂Cl₂. The combined organic layers were dried (MgSO₄). Filtration and concentration gave the mesylate as a yellow oil. The mesylate was used in step 4 without further purification.

### Step 4:

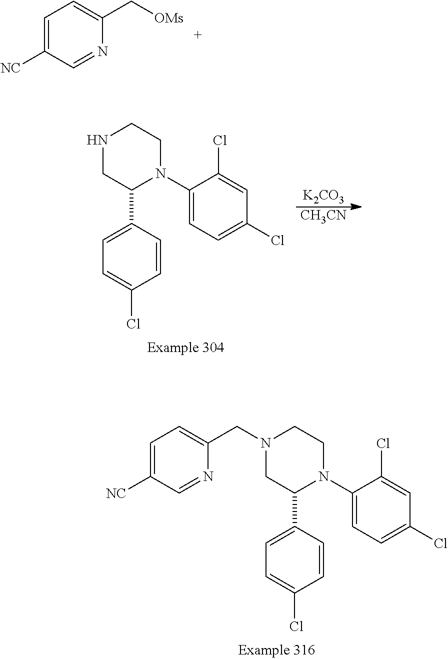

The mesylate (0.75 mmol), enantio-enriched piperazine Example 304 (150 mg), and K₂CO₃ (152 mg) were taken up in CH₃CN and heated at reflux (95° C., 1.5 h). The solution was cooled and partitioned between EtOAc and 1 N NaOH_{(aq.)}. The aqueous layer was extracted with EtOAc. The combined organic layers were washed with brine and dried (MgSO₄). Filtration and concentration gave a yellow oil. Purification via thin-layer preparative chromatography (2/1 hexanes/EtOAc, SiO₂) gave 155 mg (77%) of Example 316 as a white solid.

### Preparation of Example 317

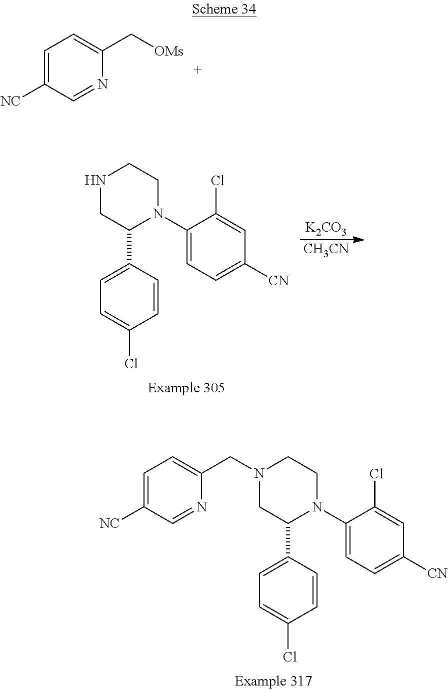

Example 317 was prepared according to the procedures described in Scheme 33, Step 4, above, except that cyano-piperazine Example 305 was used instead of Example 304.

### Preparation of Example 318

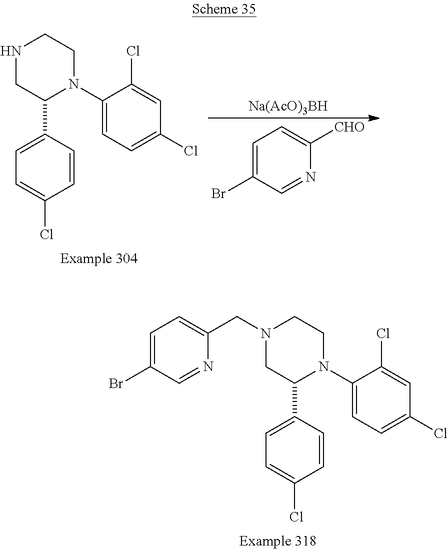

Example 318 was prepared according to the procedure described in Scheme 30 except that 5-bromo-pyridine-2-carbaldehyde was used instead of 6-bromo-pyridine-3-carbaldehyde.

### Preparation of Examples 319 and 320

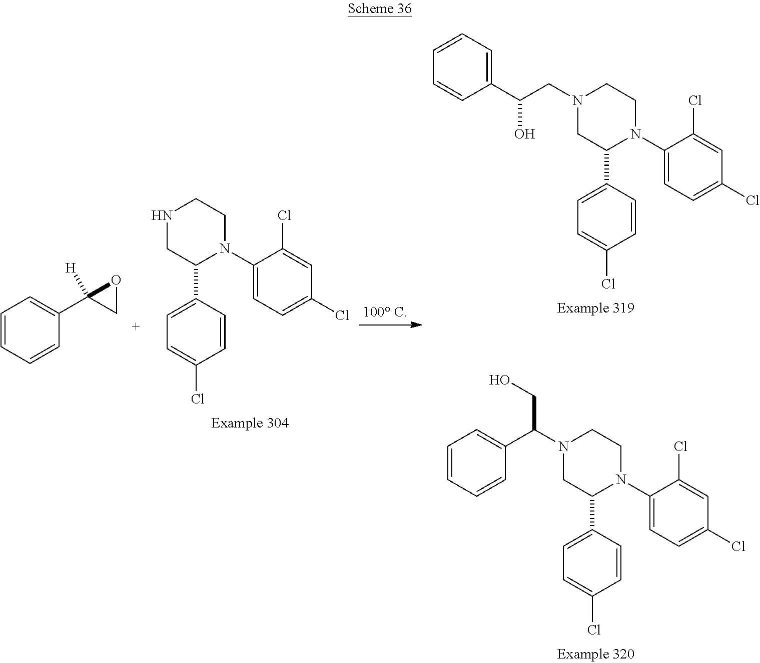

(R)-Styrene oxide (0.1 mL) and the enantio-enriched piperazine Example 304 (200 mg) were heated neat at 95° C. (4 h). The residue was purified via thin-layer preparative chromatography (3/1 hexanes/EtOAc, SiO₂) to furnish 141 mg (48%) of Example 319 and 47 mg (16%) of Example 320 as colorless oils.

### Preparation of Examples 321 and 322

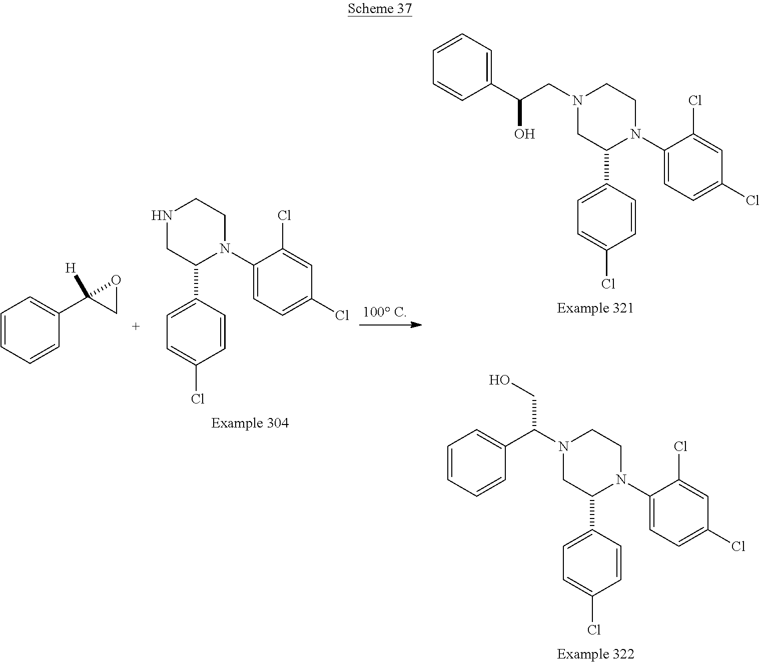

Following the procedure in Scheme 36, (S)-styrene oxide and the piperazine Example 304 gave Example 321 and Example 322.

### Preparation of Examples 323-335

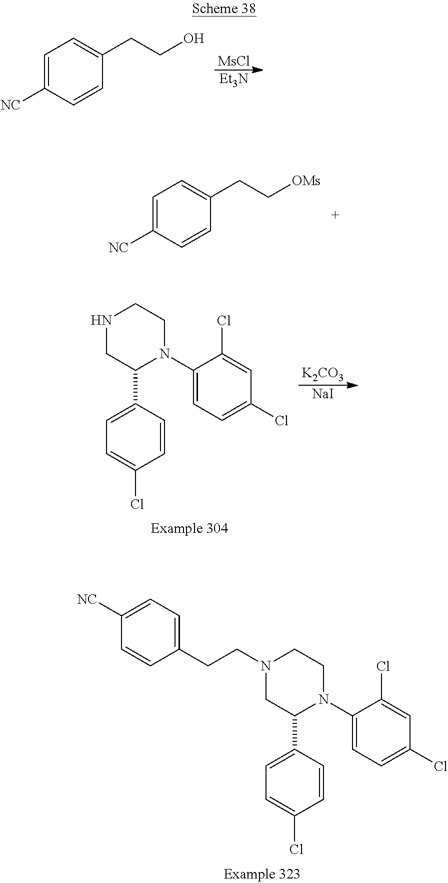

### Step 1:

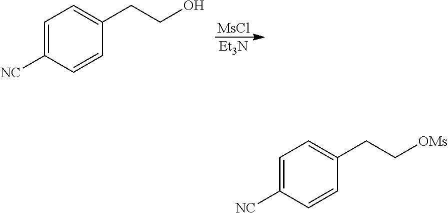 [SMILES: "Et3N" ; "MsCl" ; N#Cc1ccc(CCO)cc1 ; CS(=O)(=O)OCCc1ccc(C#N)cc1]

The 4-(2-hydroxyethyl)-benzonitrile (500 mg) and Et₃N (480 mg) were taken up in CH₂Cl₂ at 25° C. Methanesulfonyl chloride (470 mg) was added and the solution was stirred at 25° C. (0.5 h). The solution was diluted with CH₂Cl₂ and washed with saturated NaHCO_{3(aq.)}. The aqueous layer was extracted with CH₂Cl₂. The combined organic layers were dried (MgSO₄), filtered, and concentrated. The resulting mesylate was used in step 2 without further purification.

### Step 2:

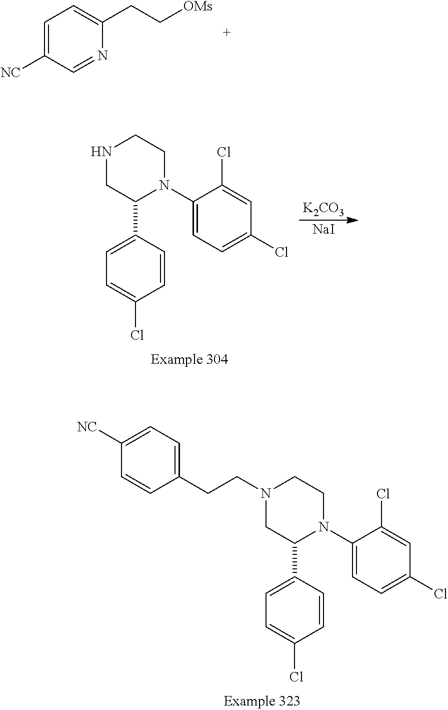

The mesylate prepared in step 1 (63 mg), piperazine Example 304 (80 mg), K₂CO₃ (97 mg), and NaI (40 mg) were taken up in CH₃CN and heated at reflux (90° C., 18 h). The solution was partitioned between EtOAc and water. The aqueous layer was extracted with EtOAc. The combined organic layers were washed with brine and dried (MgSO₄). Filtration and concentration gave a yellow oil. Purification via thin-layer preparative chromatography (7% EtOAc in CH₂Cl₂, SiO₂) gave 100 mg (90%) of Example 323 as a colorless oil.

The following examples were prepared in a similar manner using the appropriate alcohol and piperazine (Table XII).

TABLE XII

| Example # | Piperazine | Alcohol | Example Structure |
|---|---|---|---|
| 324 | 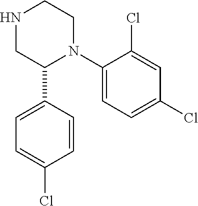 [SMILES: Clc1ccc([C@@H]2CNCCN2c2ccc(Cl)cc2Cl)cc1] | 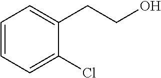 | 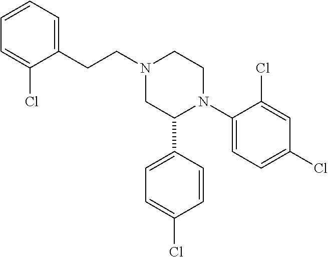 [SMILES: Clc1ccc([C@@H]2CN(CCc3ccccc3Cl)CCN2c2ccc(Cl)cc2Cl)cc1] |
| 325 | 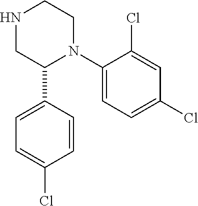 [SMILES: Clc1ccc([C@@H]2CNCCN2c2ccc(Cl)cc2Cl)cc1] | 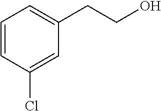 | 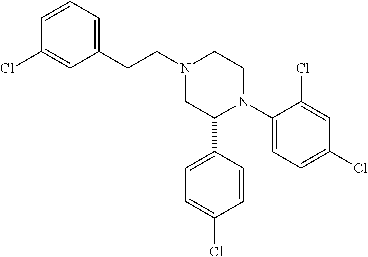 [SMILES: Clc1ccc([C@@H]2CN(CCc3cccc(Cl)c3)CCN2c2ccc(Cl)cc2Cl)cc1] |
| 326 | 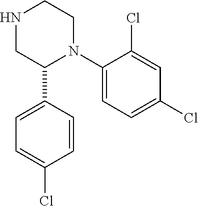 [SMILES: Clc1ccc([C@@H]2CNCCN2c2ccc(Cl)cc2Cl)cc1] | 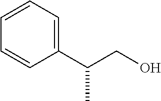 | 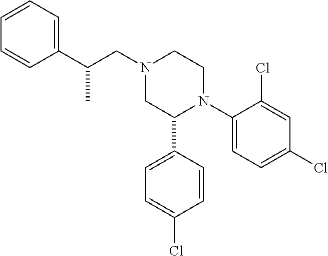 [SMILES: C[C@@H](CN1CCN(c2ccc(Cl)cc2Cl)[C@H](c2ccc(Cl)cc2)C1)c1ccccc1] |
| 327 | 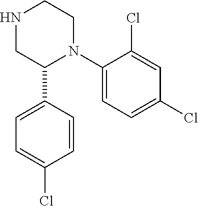 [SMILES: Clc1ccc([C@@H]2CNCCN2c2ccc(Cl)cc2Cl)cc1] | 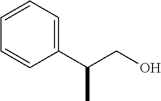 | 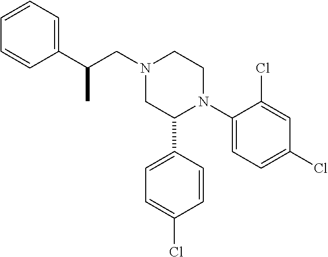 [SMILES: C[C@H](CN1CCN(c2ccc(Cl)cc2Cl)[C@H](c2ccc(Cl)cc2)C1)c1ccccc1] |
| 328 | 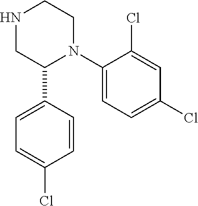 [SMILES: Clc1ccc([C@@H]2CNCCN2c2ccc(Cl)cc2Cl)cc1] | 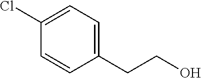 | 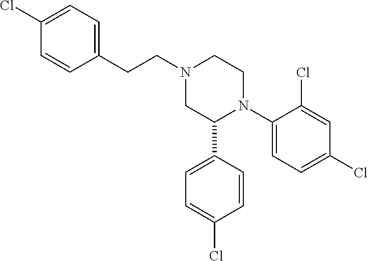 [SMILES: Clc1ccc(CCN2CCN(c3ccc(Cl)cc3Cl)[C@H](c3ccc(Cl)cc3)C2)cc1] |
| 329 | 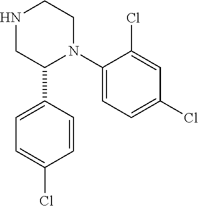 [SMILES: Clc1ccc([C@@H]2CNCCN2c2ccc(Cl)cc2Cl)cc1] | 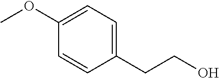 | 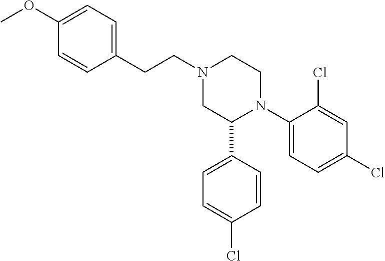 [SMILES: COc1ccc(CCN2CCN(c3ccc(Cl)cc3Cl)[C@H](c3ccc(Cl)cc3)C2)cc1] |
| 330 | 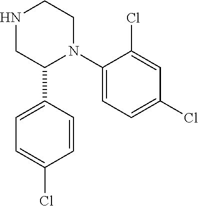 [SMILES: Clc1ccc([C@@H]2CNCCN2c2ccc(Cl)cc2Cl)cc1] | 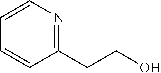 | 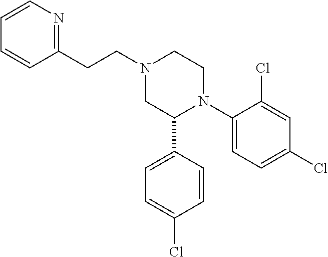 [SMILES: Clc1ccc([C@@H]2CN(CCc3ccccn3)CCN2c2ccc(Cl)cc2Cl)cc1] |
| 331 | 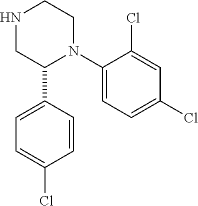 [SMILES: Clc1ccc([C@@H]2CNCCN2c2ccc(Cl)cc2Cl)cc1] | 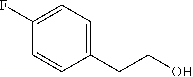 | 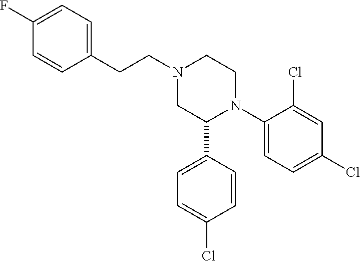 [SMILES: Fc1ccc(CCN2CCN(c3ccc(Cl)cc3Cl)[C@H](c3ccc(Cl)cc3)C2)cc1] |
| 332 | 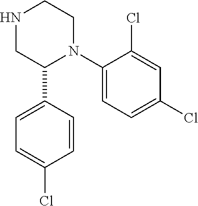 [SMILES: Clc1ccc([C@@H]2CNCCN2c2ccc(Cl)cc2Cl)cc1] | 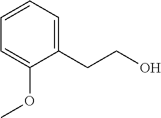 | 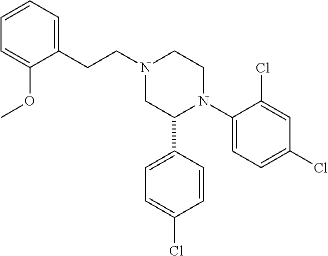 [SMILES: COc1ccccc1CCN1CCN(c2ccc(Cl)cc2Cl)[C@H](c2ccc(Cl)cc2)C1] |
| 333 | 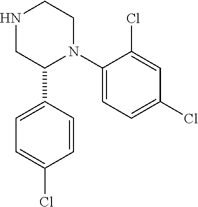 [SMILES: Clc1ccc([C@@H]2CNCCN2c2ccc(Cl)cc2Cl)cc1] | 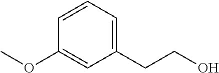 | 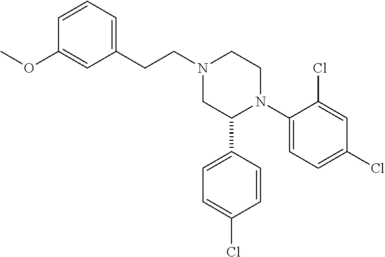 [SMILES: COc1cccc(CCN2CCN(c3ccc(Cl)cc3Cl)[C@H](c3ccc(Cl)cc3)C2)c1] |
| 334 | 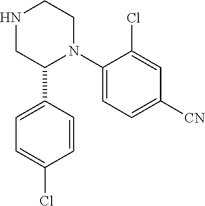 [SMILES: N#Cc1ccc(N2CCNC[C@H]2c2ccc(Cl)cc2)c(Cl)c1] | 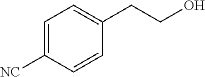 [SMILES: N#Cc1ccc(CCO)cc1] | 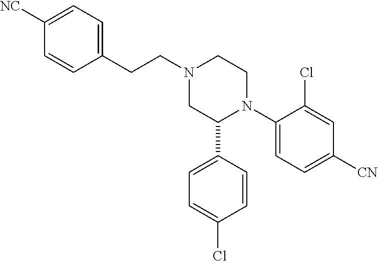 [SMILES: N#Cc1ccc(CCN2CCN(c3ccc(C#N)cc3Cl)[C@H](c3ccc(Cl)cc3)C2)cc1] |
| 335 | 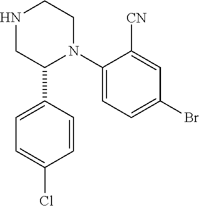 [SMILES: N#Cc1cc(Br)ccc1N1CCNC[C@H]1c1ccc(Cl)cc1] | 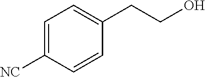 [SMILES: N#Cc1ccc(CCO)cc1] | 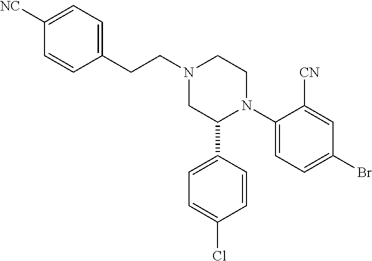 [SMILES: N#Cc1ccc(CCN2CCN(c3ccc(Br)cc3C#N)[C@H](c3ccc(Cl)cc3)C2)cc1] |

### Preparation of Example 336

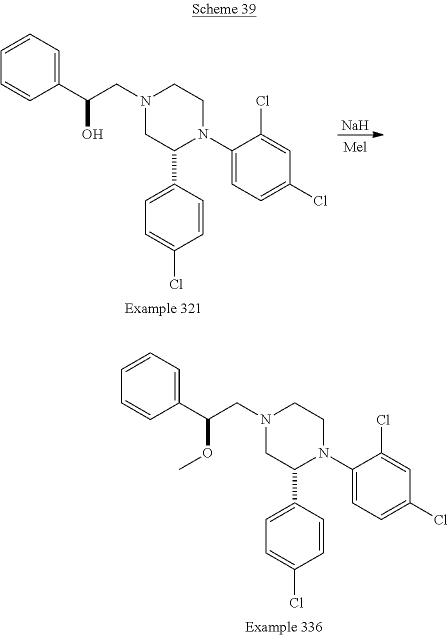

The alcohol Example 321 (90 mg) was taken up in THF. Sodium hydride (100 mg of a 60 wt % dispersion in oil) was added. After stirring at 25° C. for 10-15 minutes, iodomethane (0.05 mL) was added. The mixture was stirred at 25° C. (2 h). The solution was partitioned between EtOAc and H₂O. The aqueous layer was extracted with EtOAc. The combined organic layers were washed with brine and dried (MgSO₄). Filtration and concentration gave a yellow oil. Purification via thin-layer preparative chromatography (3/1 hexanes/EtOAc, SiO₂) gave 89 mg (98%) of Example 336 as a colorless oil.

### Preparation of Example 337

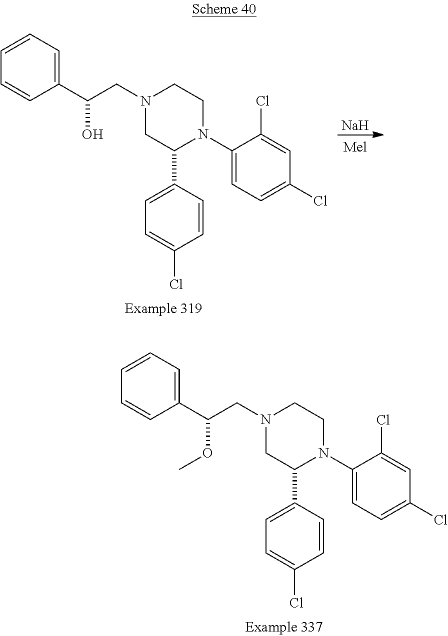

Using a procedure similar to the procedure described in Scheme 39, Example 319 was converted into Example 337.

### Preparation of Examples 338-339

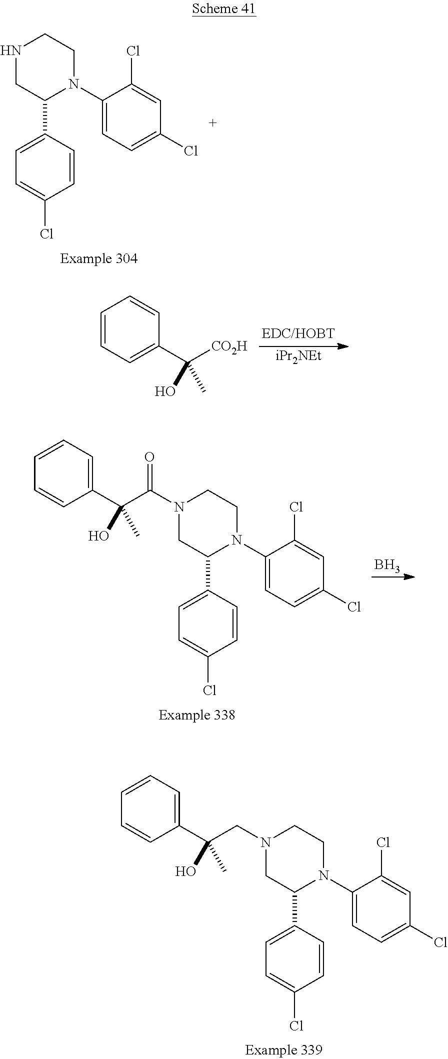

### Step 1:

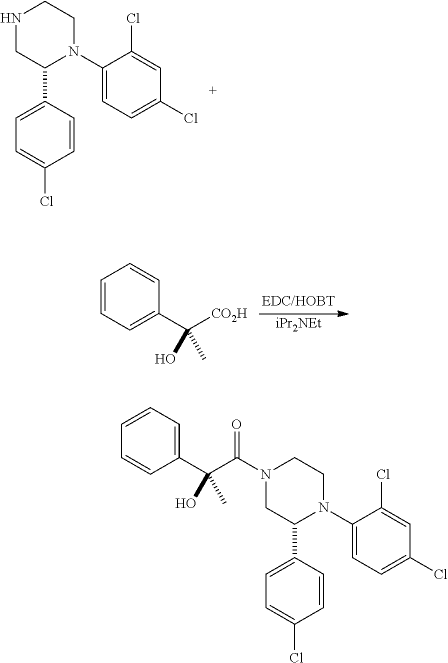

The piperazine Example 304 (300 mg), carboxylic acid (160 mg), EDC (211 mg), HOBT (149 mg), and iPr₂NEt (0.2 mL) were taken up in CH₃CN and heated at 65° C. (18 h). The solution was concentrated. The residue was partitioned between EtOAc and 1 N NaOH_{(aq.)}. The aqueous layer was extracted with EtOAc. The combined organic layers were washed with brine and dried (MgSO₄). Filtration and concentration gave a yellow oil. Purification via flash chromatography (2/1 hexanes/EtOAc, SiO₂) gave 223 mg (52%) of amide Example 338 as a colorless oil.

### Step 2:

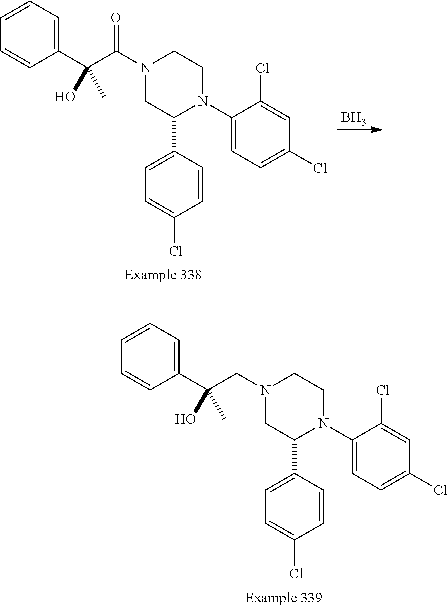

The amide Example 338 (223 mg) and BH₃-THF complex (1.0 M BH₃ in THF, 3 mL) were taken up in THF and heated at reflux (65-70° C., 18 h). The solution was cooled and quenched with MeOH (2-3 mL) and 1 M HCl_{(aq.)} (3040 mL). The solution was stirred at 25° C. for 2 h. The solution was cooled and rendered basic with NaOH pellets (pH=10-12). The mixture was extracted with EtOAc. The combined organic layers were washed with brine and dried (MgSO₄). Filtration and concentration gave a yellow oil. Purification via thin-layer preparative chromatography (6/1 hexanes/EtOAc, SiO₂) gave 133 mg (61%) of Example 339 as a colorless oil.

### Preparation of Examples 340-341

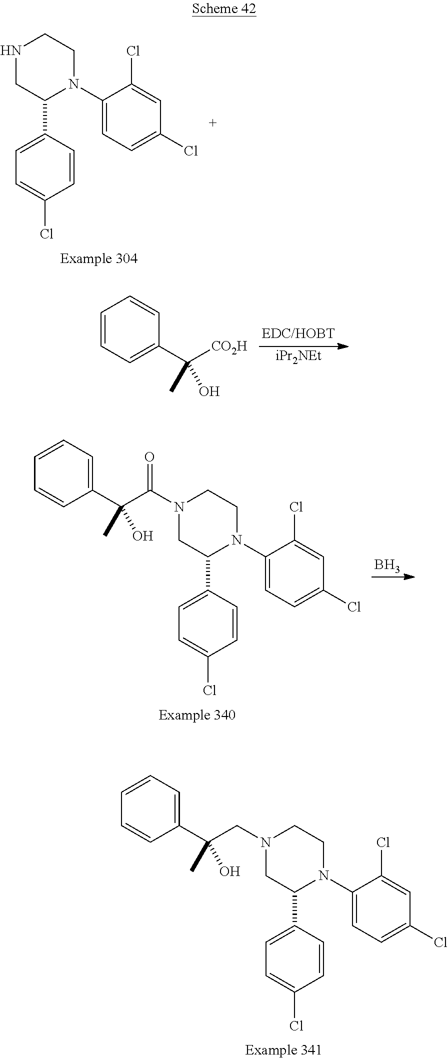

Using the procedures outlined in Scheme 41, Examples 340 and 341 were prepared from the appropriate piperazine and acid as shown in Scheme 42.

### Preparation of Examples 342-343

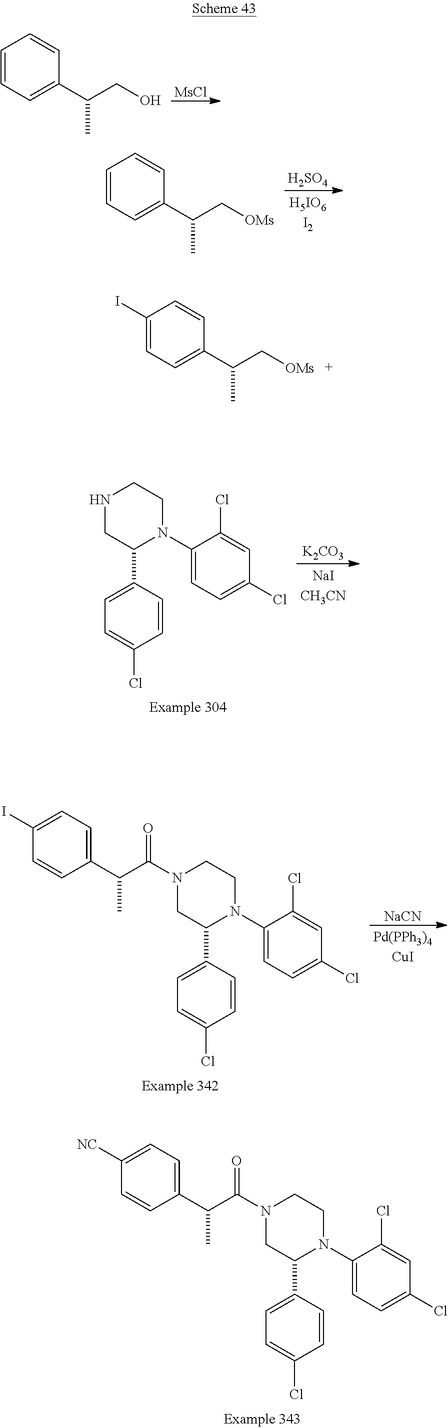

### Step 1:

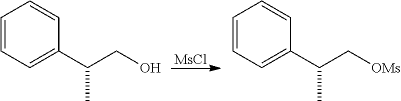

The 2-phenyl-propan-1-ol (500 mg) and Et₃N (0.6 mL) were taken up in CH₂Cl₂ and cooled to 0° C. Methanesulfonyl sulfonyl chloride (0.3 mL) was added at 0° C., and the reaction was warmed to 25° C. (3 h). The solution was diluted with CH₂Cl₂ and washed with 1 N NaOH_{(aq.)}. The aqueous layer was extracted with CH₂Cl₂. The combined organic layers were dried (MgSO₄). Filtration and concentration gave the corresponding mesylate as a yellow oil, which was used without further purification in step 2.

### Step 2:

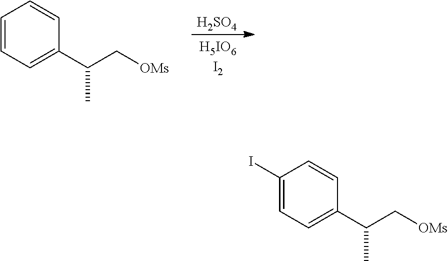

The mesylate prepared in step 1 (798 mg), H₂SO₄ (0.2 mL), H₅IO₆ (212 mg), and I₂ (436 mg) were taken up in glacial acetic acid and stirred at 25° C. (18 h). The reaction mixture was heated at 70° C. for 3 h. The solution was cooled and rendered basic with 3 N NaOH_{(aq.)}. The mixture was treated with 10% Na₂S₂O_{3(aq.)} to decolorize the I₂ color. The mixture was extracted with CH₂Cl₂. The combined organic layers were dried (MgSO₄), filtered, and concentrated. Purification via flash chromatography (4/1 hexanes/EtOAc, SiO₂) gave 800 mg (63%) of the iodide as a yellow oil.

### Step 3:

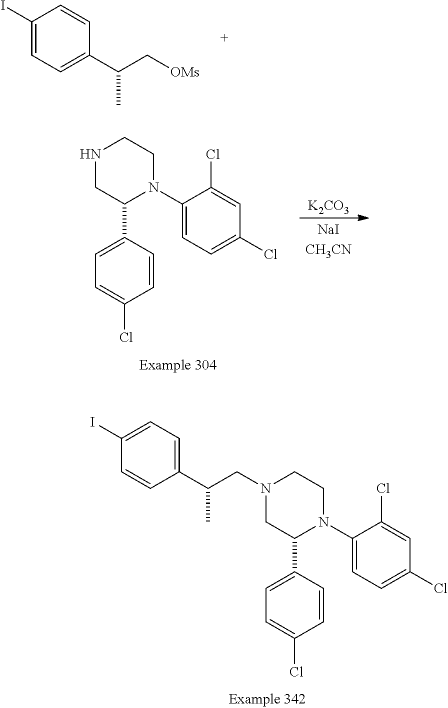

The iodide prepared in step 2 (394 mg), piperazine Example 304 (200 mg), K₂CO₃ (240 mg), and NaI (44 mg) were taken up in CH₃CN and heated (85-90° C., 18 h). The reaction mixture was partitioned between EtOAc and 1 N NaOH_{(aq.)}. The aqueous layer was extracted with EtOAc. The combined organic layers were washed with brine and dried (MgSO₄). Filtration and concentration gave a yellow oil. Purification via thin-layer preparative chromatography (8/1 hexanes/EtOAc, SiO₂) gave 118 mg (17%) of the iodo-piperazine Example 342 as a colorless foam.

### Step 4:

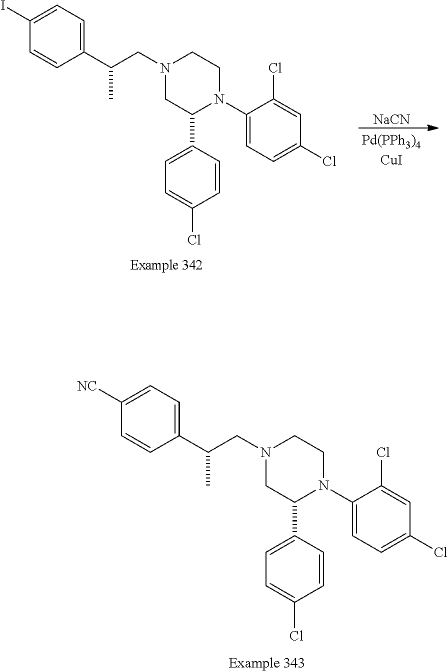 [SMILES: C[C@@H](CN1CCN(c2ccc(Cl)cc2Cl)[C@H](c2ccc(Cl)cc2)C1)c1ccc(I)cc1 ; "NaCN" ; C[C@@H](CN1CCN(c2ccc(Cl)cc2Cl)[C@H](c2ccc(Cl)cc2)C1)c1ccc(C#N)cc1]

The iodo-piperazine Example 342 prepared in step 3 (118 mg), NaCN (20 mg), Pd(PPh₃)₄ (23 mg), and CuI (8 mg) were taken up in degassed EtCN and heated at 105° C. for 1 h. The solution was partitioned between EtOAc and 1 N NaOH_{(aq.)}. The aqueous layer was extracted with EtOAc. The combined organic layers were washed with brine and dried (MgSO₄). Filtration and concentration gave a yellow oil. Purification via preparative thin-layer chromatography (6/1/hexanes/EtOAc, SiO₂) gave 58 mg (59%) of Example 343.

### Preparation of Examples 344-346

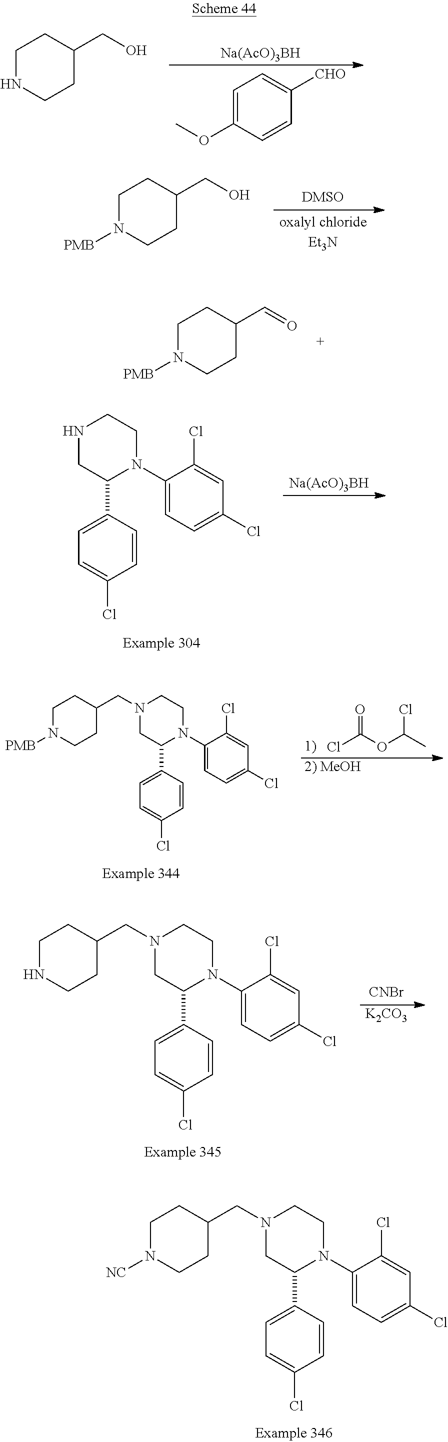

### Step 1:

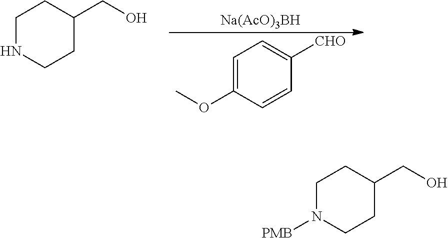

Piperidine-4-yl-methanol (5 g), p-anisaldehyde (6.3 mL), and Na(AcO)₃BH (11 g) were taken up in CH₂Cl₂ and stirred at 25° C. (18 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. The residue was partitioned between Et₂O and 1 M HCl_{(aq.)}. The aqueous layer was extracted with Et₂O. The aqueous layer was cooled to 0° C. and rendered basic via addition of NaOH pellets (pH=10-12). The mixture was extracted with CH₂Cl₂. The combined organic layers were dried (MgSO₄), filtered, and concentrated which furnished the PMB alcohol (6.35 g, 62%) as a yellow oil.

### Step 2:

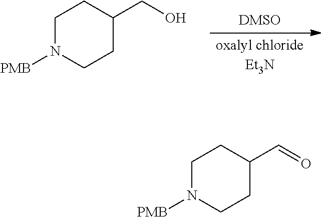

DMSO (2.5 mL) was taken up in CH₂Cl₂ (150 mL) and cooled to −40° C. (CH₃CN/CO₂). Oxalyl chloride (3.1 mL) in CH₂Cl₂ (15 mL) was added dropwise to the solution at −40° C. The solution was stirred at −40° C. for 30 minutes. The PMB alcohol prepared in step 1 (6.35 g) in CH₂Cl₂ (15 mL) was added to the solution at −40° C. The resulting solution was stirred at −40° C. for 30 minutes. Triethylamine (11.3 mL) was added to the solution at −40° C., and the resulting slurry was warmed to 25° C. and stirred at that temperature for 1.5 h. The solution was diluted with CH₂Cl₂ and washed with 1 N NaOH_{(aq.)}. The aqueous layer was extracted with CH₂Cl₂. The combined organic layers were dried (MgSO₄), filtered, and concentrated to furnish the aldehyde, which was used without further purification in step 3.

### Step 3:

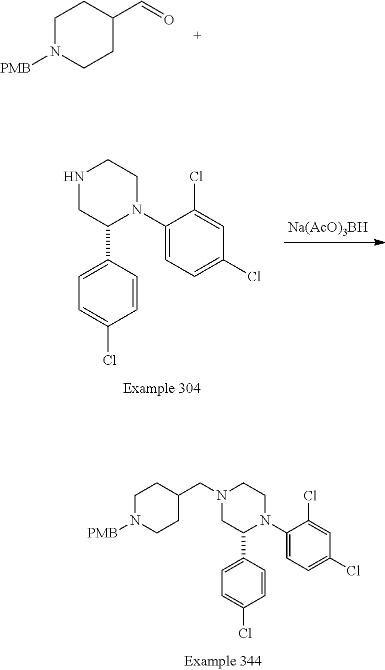

The piperazine Example 304 (500 mg), the aldehyde prepared in step 2 (440 mg), and Na(AcO)₃BH (400 mg) were taken up in CH₂Cl₂ and stirred at 25° C. (18 h). The solution was diluted with CH₂Cl₂ and washed with 1 N NaOH_{(aq.)}. The aqueous layer was extracted with CH₂Cl₂. The combined organic layers were dried (MgSO₄), filtered, and concentrated. Purification via flash chromatography (3/1 hexanes/EtOAc, SiO₂) gave 640 mg (78%) of Example 344 as a colorless oil.

### Step 4:

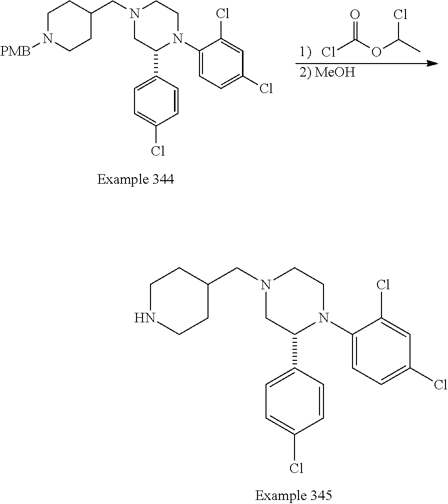

Example 344 (640 mg) and the chloro-formate shown above in step 4 (0.2 mL) were taken up in CH₂Cl₂ and stirred at 25° C. (18 h). The solution was concentrated. The residue was taken up in MeOH and heated at reflux (65° C., 2.5 h). The solution was concentrated, and the residue was partitioned between 1 M HCl_{(aq.)} and Et₂O. The aqueous layer was extracted with Et₂O. The aqueous layer was cooled (0° C.) and made basic via addition of NaOH pellets (pH=10-12). The solution was extracted with CH₂Cl₂. The combined organic layers were dried (MgSO₄), filtered, and concentrated to give Example 345 (367 mg, 74%) as a yellow oil.

### Step 5

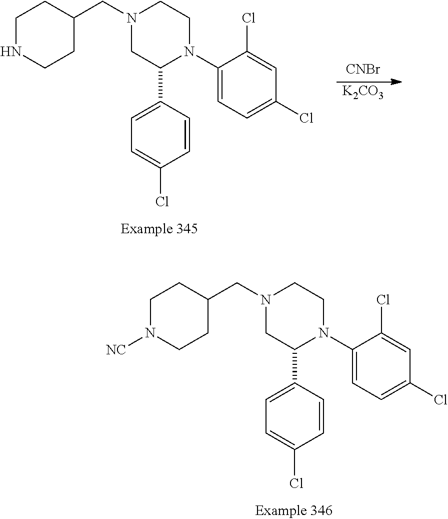

Example 345 (70 mg), CNBr (0.2 mL of a 3.0 M solution in CH₂Cl₂), and K₂CO₃ (66 mg) were taken up in CH₃CN and stirred at 25° C. (4 h). The solution was partitioned between EtOAc and saturated NaHCO_{3(aq.)}. The aqueous layer was extracted with EtOAc. The combined organic layers were washed with brine and dried (MgSO₄). Filtration and concentration gave a yellow oil. Purification via thin-layer preparative chromatography (3/1 hexanes/EtOAc, SiO₂) gave Example 346 (25 mg, 34%) as a colorless oil.

### Preparation of Example 347

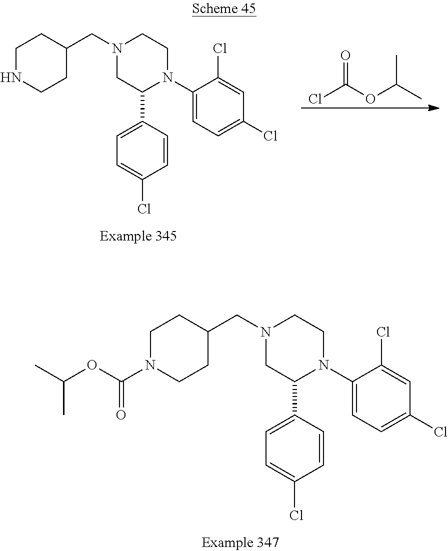

Example 345 (70 mg) and the chloro-formate shown above in scheme 16 (0.3 mL) were partitioned between CH₂Cl₂ and 1 N NaOH_{(aq.)}. The mixture was stirred at 25° C. (4 h). The mixture was diluted with water and CH₂Cl₂. The aqueous layer was extracted with CH₂Cl₂. The combined organic layers were dried (MgSO4), filtered, and concentrated. Purification via thin-layer preparative chromatography (3/1 hexanes/EtOAc, SiO₂) gave Example 347 (63 mg, 75%) as a colorless oil.

### Preparation of Example 348

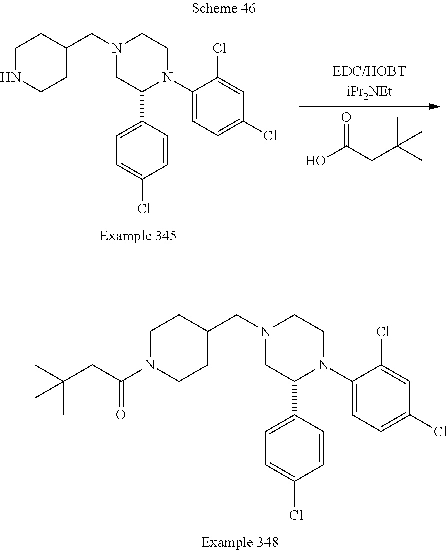

Example 345 was converted into Example 348 following the procedure outlined in Step 1 of Scheme 41 using the appropriate piperidine and acid shown in Scheme 46.

### Preparation of Example 349

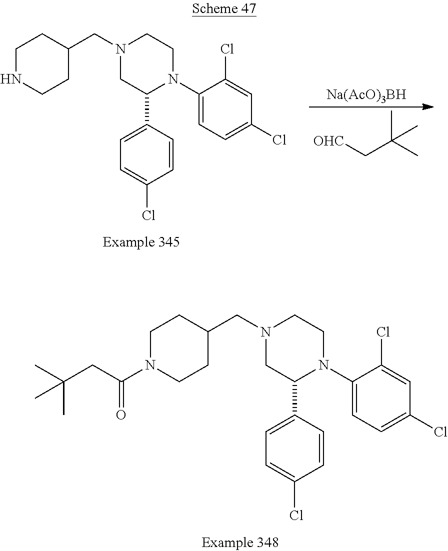

Example 345 was converted into Example 349 following the procedure outlined in Step 3 of Scheme 44 using the appropriate piperidine and aldehyde shown in Scheme 47.

### Preparation of Example 350

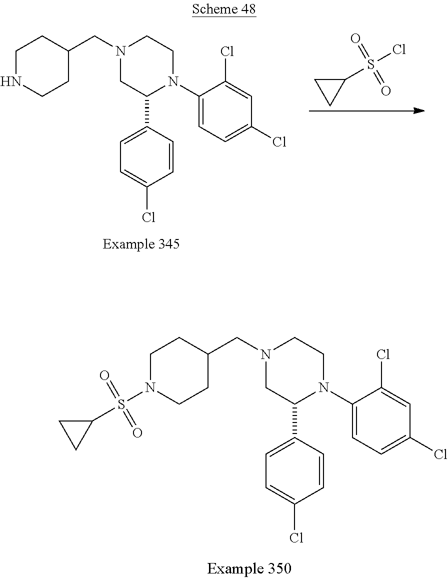 [SMILES: Clc1ccc([C@@H]2CN(CC3CCNCC3)CCN2c2ccc(Cl)cc2Cl)cc1 ; O=S(=O)(Cl)C1CC1 ; O=S(=O)(C1CC1)N1CCC(CN2CCN(c3ccc(Cl)cc3Cl)[C@H](c3ccc(Cl)cc3)C2)CC1]

Example 345 was converted into Example 350 following the procedure outlined in Step 8 of Scheme 14 using the appropriate piperidine and sulfonyl chloride shown in Scheme 48.

### Preparation of Example 351

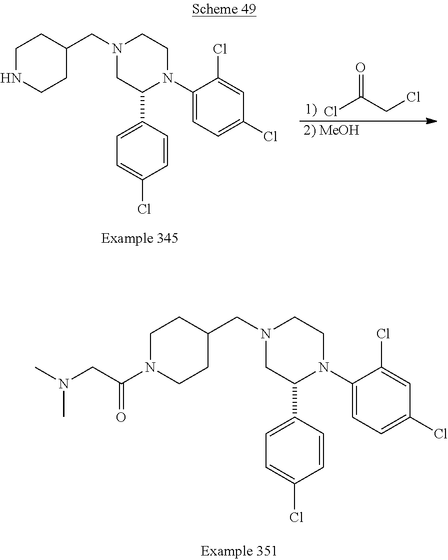

Example 345 (45 mg) and the acid chloride (0.05 mL) were partitioned between CH₂Cl₂ and saturated NaHCO_{3(aq.)}. The mixture was stirred at 25° C. (3 h). The layers were separated, and the aqueous layer was extracted with CH₂Cl₂. The organic layers were combined, dried (MgSO₄), filtered, and concentrated to furnish the chloro-amide. The chloro-amide was taken up in DMF and 20 mL of a 2.0 M Me₂NH in THF solution was added. The solution was heated in a sealed tube (75° C., 66 h). The solution was concentrated. The residue was partitioned between CH₂Cl₂ and 1 N NaOH_{(aq.)}. The aqueous layer was extracted with CH₂Cl₂. The combined organic layers were dried (MgSO₄), filtered, and concentrated. Purification via thin-layer preparative chromatography (20/1 CH₂Cl₂/MeOH, SiO₂) gave 18 mg (34%) of Example 351 as a colorless oil.

### Preparation of Examples 352-354

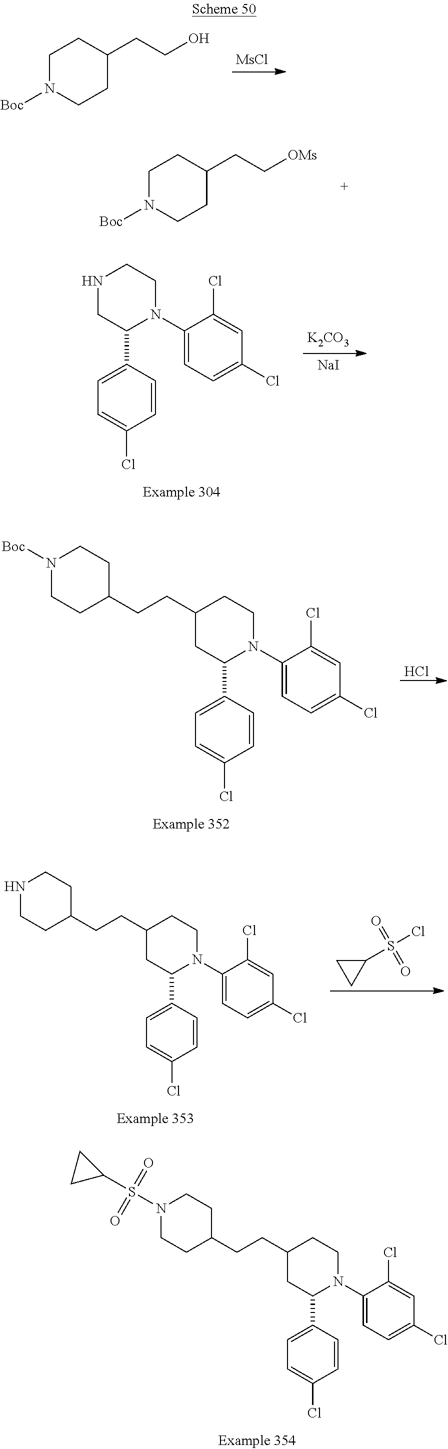

### Step 1:

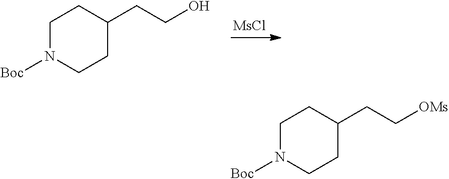 [SMILES: "MsCl" ; CC(C)(C)OC(=O)N1CCC(CCO)CC1 ; CC(C)(C)OC(=O)N1CCC(CCOS(C)(=O)=O)CC1]

The N-Boc alcohol shown above in Scheme 50 was converted into the mesylate according to the procedure outlined in Step 1 of Scheme 43.

### Step 2:

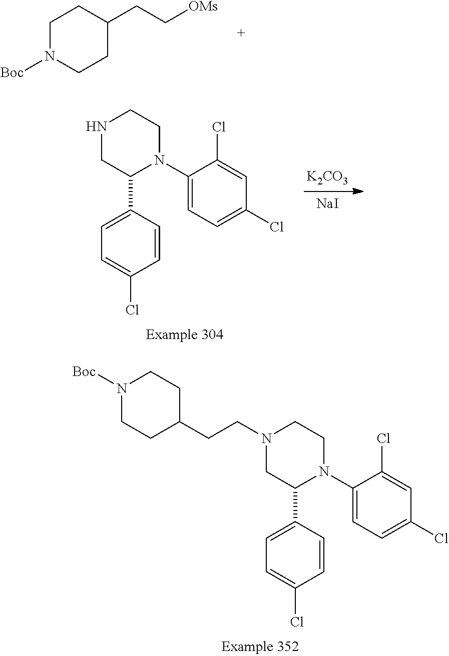

Example 352 was prepared according to the procedure outlined in Step 3 of Scheme 43 using the appropriate reagents.

### Step 3:

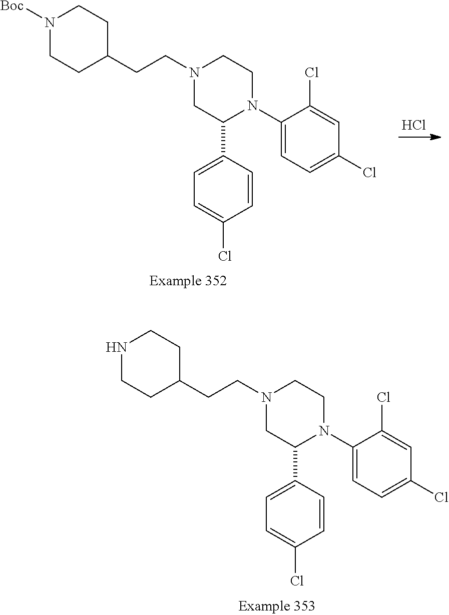 [SMILES: CC(C)(C)OC(=O)N1CCC(CCN2CCN(c3ccc(Cl)cc3Cl)[C@H](c3ccc(Cl)cc3)C2)CC1 ; Cl ; Clc1ccc([C@@H]2CN(CCC3CCNCC3)CCN2c2ccc(Cl)cc2Cl)cc1]

Example 352 (750 mg) and 4 M HCl_{(aq.)} were taken up in MeOH and stirred at 25° C. (18 h). The solution was concentrated. The residue was taken up in EtOAc and washed with 1 N NaOH_{(aq.)}. The aqueous layer was extracted with EtOAc. The combined organic layers were washed with brine and dried (MgSO₄). Filtration and concentration gave Example 353 as a yellow oil.

### Step 4:

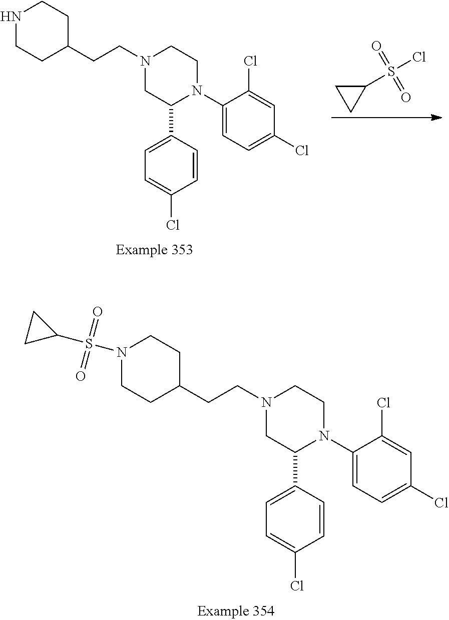 [SMILES: Clc1ccc([C@@H]2CN(CCC3CCNCC3)CCN2c2ccc(Cl)cc2Cl)cc1 ; O=S(=O)(Cl)C1CC1 ; O=S(=O)(C1CC1)N1CCC(CCN2CCN(c3ccc(Cl)cc3Cl)[C@H](c3ccc(Cl)cc3)C2)CC1]

Example 353 was converted into Example 354 using the procedures outlined in Step 1 of Scheme 43 using the appropriate reagents.

### Preparation of Example 355

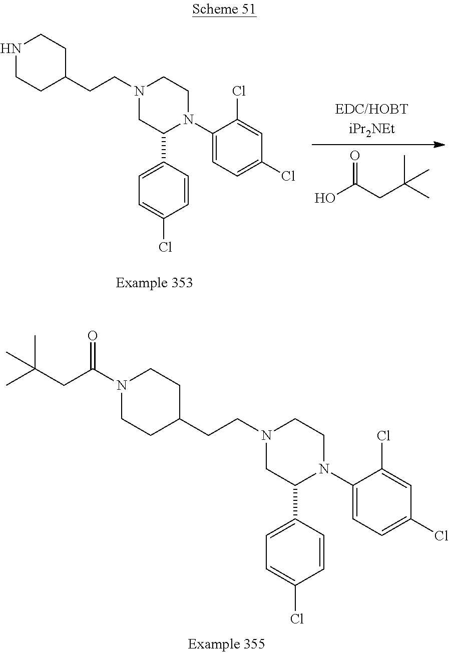

Example 353 was converted into Example 355 using the procedure outlined in Scheme 46 using the appropriate reagents.

### Preparation of Example 356

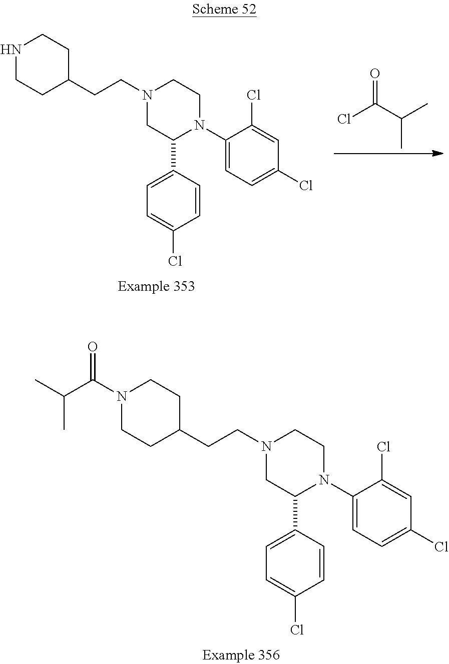

Example 353 was converted into Example 356 using the procedure outlined in Scheme 45 using the appropriate reagents.

### Preparation of Example 357

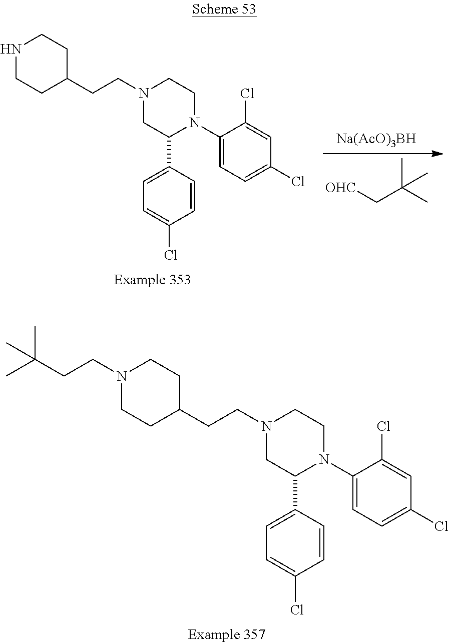

Example 353 was converted into Example 357 using the procedure outlined in Step 1 of Scheme 44 using the appropriate reagents.

### Preparation of Example 358

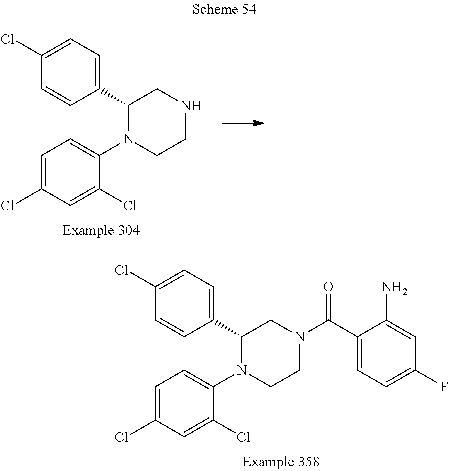 [SMILES: Clc1ccc([C@@H]2CNCCN2c2ccc(Cl)cc2Cl)cc1 ; Nc1cc(F)ccc1C(=O)N1CCN(c2ccc(Cl)cc2Cl)[C@H](c2ccc(Cl)cc2)C1]

To a solution of the piperazine Example 304 in MeCN (3 mL) was added 2-amino-4-fluorobenzoic acid (54 mg, 0.35 mmol), EDCI (67 mg, 0.35 mmol), HOBt (47 mg, 0.35 mmol) and iPr₂NEt (160 uL, 0.92 mmol). The solution was allowed to stir at room temperature overnight. The solution was then concentrated. The crude product was partitioned between EtOAc and 1M NaOH. The aqueous layer was extracted with EtOAc (3×). The combined organic layers were washed with brine, dried over Na₂SO₄, filtered and concentrated. The crude product was purified by preparative TLC (SiO₂, 2:1 hexanes:EtOAc) to afford Example 358 (99 mg). The product was converted to its HCl salt by dissolving in CH₂Cl₂ followed by the addition of 2N HCl (in ether). The solvent was then removed to provide the salt.

### Preparation of Example 359

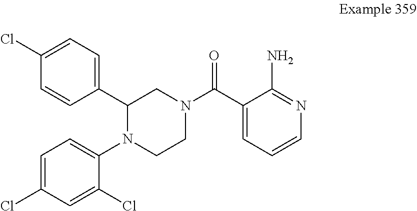 [SMILES: Nc1ncccc1C(=O)N1CCN(c2ccc(Cl)cc2Cl)C(c2ccc(Cl)cc2)C1]

Example 359 was prepared using a procedure similar to that used to prepare Example 358 except that 2-aminonicotinic acid was coupled with Example 1 instead of 2-amino-4-fluorobenzoic acid.

### Preparation of Example 360

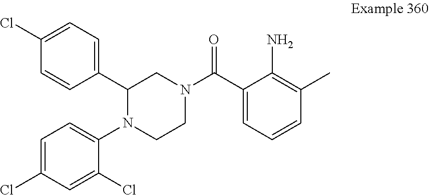 [SMILES: Cc1cccc(C(=O)N2CCN(c3ccc(Cl)cc3Cl)C(c3ccc(Cl)cc3)C2)c1N]

Example 360 was prepared using a procedure similar to that used to prepare Example 359 except that 2-amino-3-methylbenzoic acid was coupled with Example 1 instead of 2-amino-4-fluorobenzoic acid.

### Preparation of Example 361

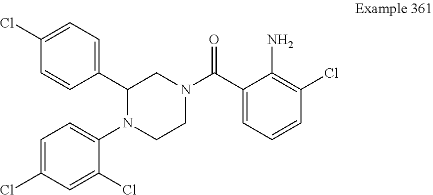 [SMILES: Nc1c(Cl)cccc1C(=O)N1CCN(c2ccc(Cl)cc2Cl)C(c2ccc(Cl)cc2)C1]

Example 361 was prepared using a procedure similar to that used to prepare Example 359 except that 2-amino-3-chlorobenzoic acid was coupled with Example 1 instead of 2-amino-4-fluorobenzoic acid.

### Preparation of Example 362

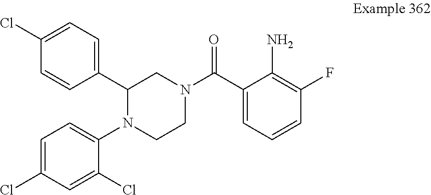 [SMILES: Nc1c(F)cccc1C(=O)N1CCN(c2ccc(Cl)cc2Cl)C(c2ccc(Cl)cc2)C1]

Example 362 was prepared using a procedure similar to that used to prepare Example 359 except that 2-amino-3-fluorobenzoic acid was coupled with Example 1 instead of 2-amino-4-fluorobenzoic acid.

### Preparation of Example 363

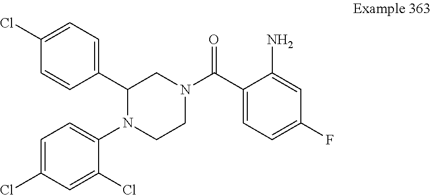 [SMILES: Nc1cc(F)ccc1C(=O)N1CCN(c2ccc(Cl)cc2Cl)C(c2ccc(Cl)cc2)C1]

Example 363 was prepared using a procedure similar to that used to prepare Example 359 except that 2-amino-4-fluorobenzoic acid was coupled with Example 1 instead of 2-amino-4-fluorobenzoic acid.

### Preparation of Example 364

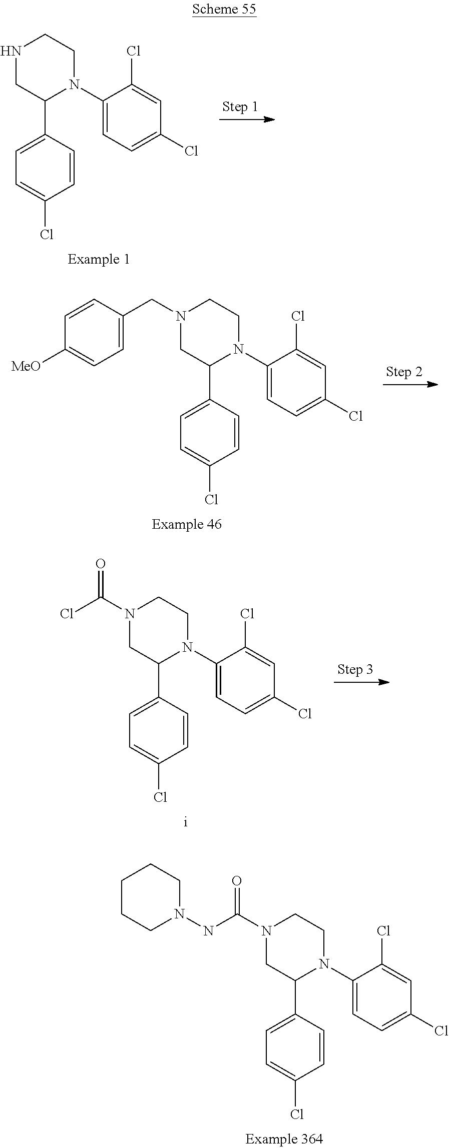 [SMILES: Clc1ccc(C2CNCCN2c2ccc(Cl)cc2Cl)cc1 ; COc1ccc(CN2CCN(c3ccc(Cl)cc3Cl)C(c3ccc(Cl)cc3)C2)cc1 ; O=C(Cl)N1CCN(c2ccc(Cl)cc2Cl)C(c2ccc(Cl)cc2)C1 ; O=C(NN1CCCCC1)N1CCN(c2ccc(Cl)cc2Cl)C(c2ccc(Cl)cc2)C1]

### Step 1:

To a solution of Example 1 (305 mg, 0.89 mmol) in DCE (5 mL) was added p-anisaldehyde (134 mg, 0.98 mmol), sodium triacetoxyborohydride (208 mg, 0.98 mmol) and acetic acid (59 mg, 0.98 mmol). The solution was stirred at room temperature overnight. The mixture was diluted with CH₂Cl₂ and washed with 1 M NaOH (aq.). The aqueous layer was extracted with CH₂Cl₂ (3×). The combined organic layers were dried over Na₂SO₄, filtered and concentrated. The crude product purified by flash chromatography (SiO₂, gradient 100:0 to 60:40 hexanes:EtOAc) to afford Example 46 (180 mg).

### Step 2:

To a solution of Example 46 (165 mg, 0.36 mmol) in CH₂Cl₂(5 mL) at 0° C. was added triphosgene (33 mg, 0.125 mmol) in CH₂Cl₂ (2 mL). The solution was stirred at 0° C. for 2 h. The solution was then concentrated in vacuo to afford the crude carbamoyl chloride which was used without purification in step 3.

### Step 3:

To a solution of the carbamoyl chloride prepared in step 2 (0.36 mmol) in CH₂Cl₂ (5 mL) was added 1-amino piperidine (40 mg, 0.40 mmol) and iPr₂NEt (52 mg, 0.40 mmol). The solution was stirred at room temperature overnight. The solution was diluted with CH₂Cl₂ and washed with NaHCO₃ (aq.). The aqueous layer was extracted with CH₂Cl₂ (3×). The combined organic layers were dried over Na₂SO₄, filtered and concentrated. The crude product was purified by preparative TLC (SiO₂; 2:1 EtOAc:hexanes) to afford Example 364 (38 mg). The product was converted to the HCl salt by dissolving it in CH₂Cl₂ followed by the addition of 2N HCl (in ether). The solvent was then removed to provide the salt.

### Preparation of Example 365

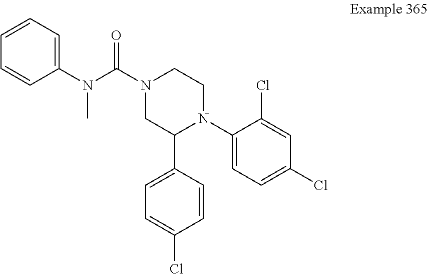 [SMILES: CN(C(=O)N1CCN(c2ccc(Cl)cc2Cl)C(c2ccc(Cl)cc2)C1)c1ccccc1]

Example 365 was prepared from carbamoyl chloride i (Scheme 55, step 2) using a procedure similar to that used to prepare Example 364, except that N-methylaniline was used in Step 3 (above) instead of 1-aminopiperidine.

### Preparation of Example 366

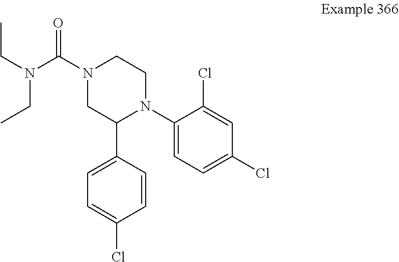 [SMILES: CCN(CC)C(=O)N1CCN(c2ccc(Cl)cc2Cl)C(c2ccc(Cl)cc2)C1]

Example 366 was prepared from carbamoyl chloride i (Scheme 55, Step 2) using a procedure similar to that used to prepare Example 364, except that diethylamine was used in Step 3 (above) instead of 1-aminopiperidine.

### Preparation of Example 367

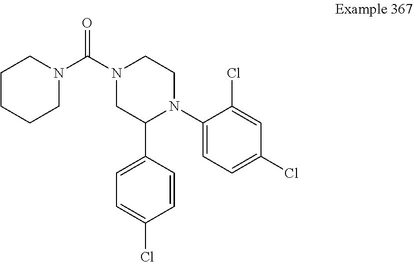 [SMILES: O=C(N1CCCCC1)N1CCN(c2ccc(Cl)cc2Cl)C(c2ccc(Cl)cc2)C1]

Example 367 was prepared from carbamoyl chloride i (Scheme 55, Step 2) using a procedure similar to that used to prepare Example 364, except that piperidine was used in Step 3 (above) instead of 1-aminopiperidine.

### Preparation of Example 368

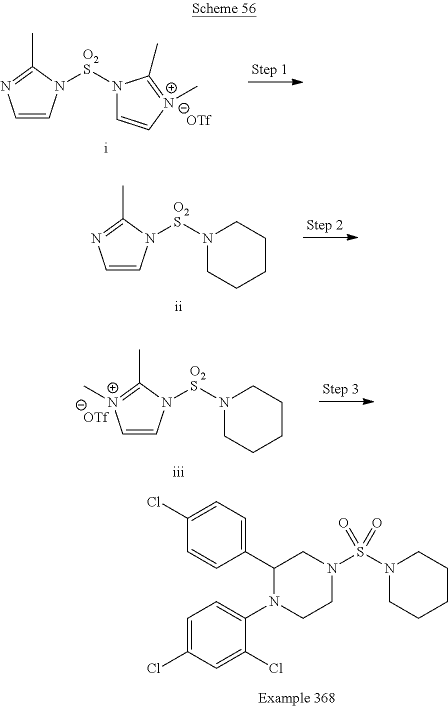

### Step 1:

To a solution of salt i (method of J. Organic Chem 68, (2003) 115-119; herein incorporated by reference) (233 mg, 0.64 mmol) in MeCN (5 mL) was added piperidine (37 mg, 0.43 mmol). The solution was allowed to stir overnight at room temperature. The solution was then concentrated and the crude product was purified by filtration through a SiO₂ plug using EtOAc to wash the plug. The filtrate was concentrated to afford ii (88 mg) as a white crystalline solid.

### Step 2:

To a solution of ii (88 mg, 0.38 mmol) in CH₂Cl₂ (5 mL) at 0° C. was added methyl triflate (69 mg, 0.42 mmol). The solution was stirred at 0° C. for 2 h. The solution was concentrated to afford iii as a white solid.

### Step 3:

To a solution of iii (0.38 mmol) in MeCN (2 mL) was added Example 1 (100 mg, 0.29 mmol). The solution was heated to reflux for 16 h. The solution was then concentrated and purified by flash chromatography (SiO₂; gradient elution 100:0 to 80:20 hexanes:EtOAc) to afford Example 368 (150 mg) as a clear oil. The product was converted to the HCl salt by dissolving in CH₂Cl₂ followed by the addition of 2N HCl (in ether). The solvent was then removed to provide the salt.

### Preparation of Example 369

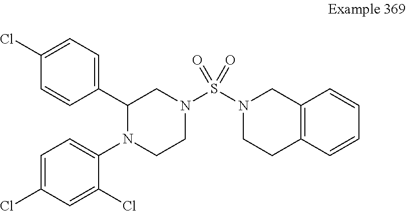 [SMILES: O=S(=O)(N1CCc2ccccc2C1)N1CCN(c2ccc(Cl)cc2Cl)C(c2ccc(Cl)cc2)C1]

Example 369 was prepared from i (Scheme 56, above) using a procedure similar to that used to prepare Example 368, except that tetrahydroquinoline was used in Step 1 (above) instead of piperidine.

### Preparation of Examples 370-371

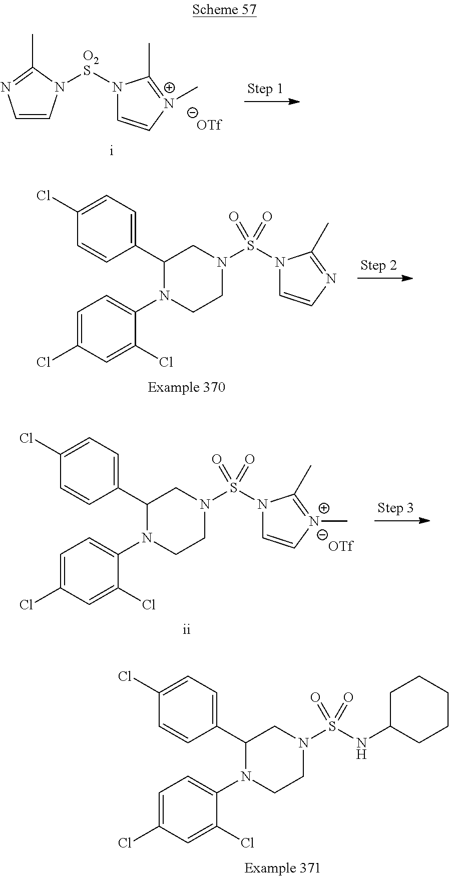 [SMILES: Cc1nccn1S(=O)(=O)n1cc[n+](C)c1C ; Cc1nccn1S(=O)(=O)N1CCN(c2ccc(Cl)cc2Cl)C(c2ccc(Cl)cc2)C1 ; Cc1n(S(=O)(=O)N2CCN(c3ccc(Cl)cc3Cl)C(c3ccc(Cl)cc3)C2)cc[n+]1C ; O=S(=O)(NC1CCCCC1)N1CCN(c2ccc(Cl)cc2Cl)C(c2ccc(Cl)cc2)C1 ; "OTf"]

### Step 1:

To a solution of salt i (555 mg, 1.53 mmol) in MeCN (10 mL) was added Example 1 (349 mg, 1.02 mmol). The solution was allowed to stir overnight at room temperature. The solution was concentrated and the crude product was purified via flash chromatography (SiO₂; gradient elution 100:0 to 1:1 hexanes:EtOAc) to afford Example 370 (252 mg) as a white crystalline solid.

### Step 2:

To a solution of Example 370 (252 mg, 0.52 mmol) in CH₂Cl₂ (15 mL) at 0° C. was added methyl triflate (89 mg, 0.54 mmol). The solution was stirred at 0° C. for 2 h. The solution was concentrated to afford ii as a white solid.

### Step 3:

To a solution of ii (0.17 mmol) in MeCN (2 mL) was added aminocyclohexane (17 mg, 0.17 mmol). The solution was heated to reflux for 16 h. The solution was then concentrated and purified by preparative TLC (SiO₂ 2:1 hexanes:EtOAc) to afford Example 371 (45 mg) as a clear oil. The product was converted to the HCl salt by dissolving in CH₂Cl₂ followed by the addition of 2N HCl (in ether). The solvent was then removed to provide the salt.

### Preparation of Example 372

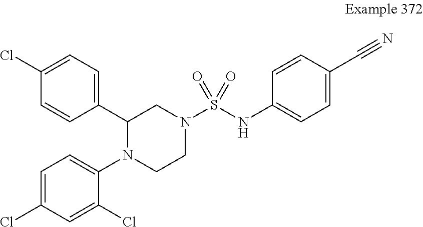 [SMILES: N#Cc1ccc(NS(=O)(=O)N2CCN(c3ccc(Cl)cc3Cl)C(c3ccc(Cl)cc3)C2)cc1]

Example 372 was prepared using a procedure similar to that used to prepare Example 371, except 4-cyanoaniline was used instead of aminocyclohexane in Step 3 (above).

### Preparation of Example 373

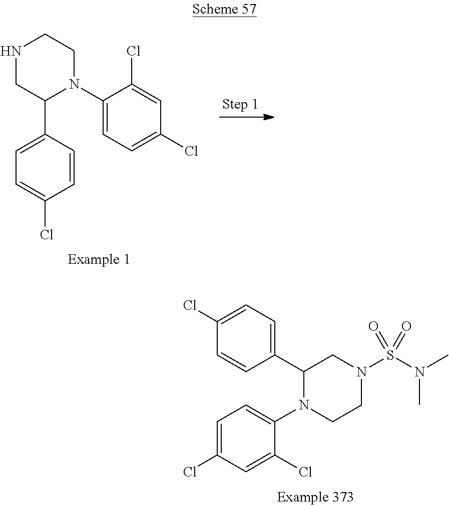 [SMILES: Clc1ccc(C2CNCCN2c2ccc(Cl)cc2Cl)cc1 ; CN(C)S(=O)(=O)N1CCN(c2ccc(Cl)cc2Cl)C(c2ccc(Cl)cc2)C1]

To a solution of Example 1 (70 mg, 0.20 mmol) in CH₂Cl₂ (2 mL) was added N,N-dimethyl amino sulfonyl chloride (32 mg, 0.23 mol) and iPr₂NEt (31 mg, 0.24 mmol). The solution was stirred at room temperature overnight. The solution was then diluted with CH₂Cl₂ The solution was diluted with CH₂Cl₂ and washed with NaHCO₃ (aq.). The aqueous layer was extracted with CH₂Cl₂ (3×). The combined organic layers were dried over Na₂SO₄, filtered and concentrated. The crude product was purified by preparative TLC (SiO₂; 2:1 EtOAc:hexanes) to afford Example 373 (66 mg). The product was converted to the HCl salt by dissolving in CH₂Cl₂ followed by the addition of 2N HCl (in ether). The solvent was then removed to provide the salt.

### Preparation of Examples 374-375

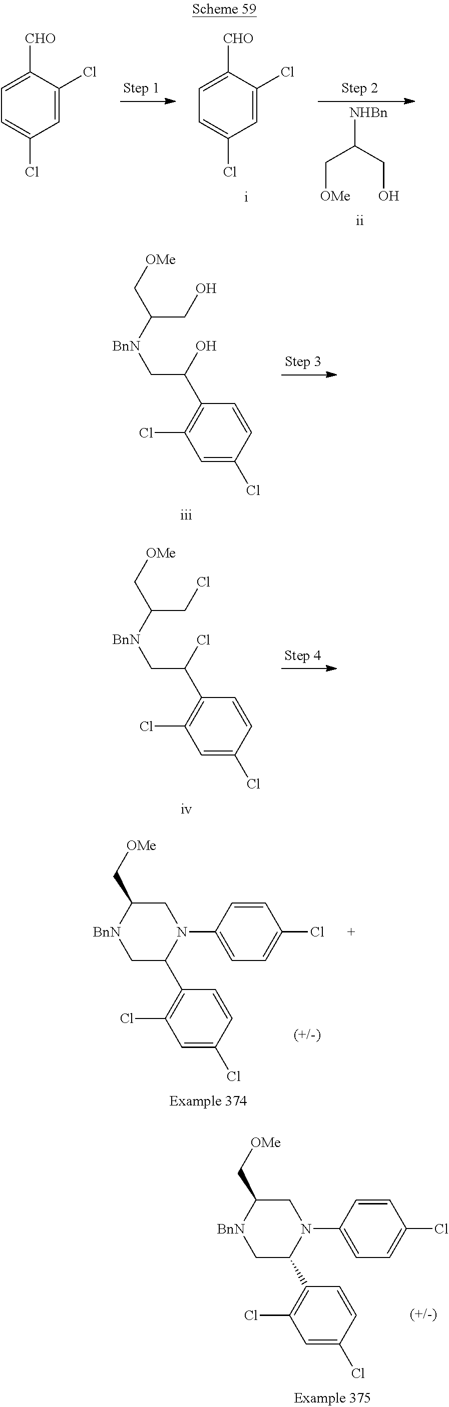

### Step 1:

To a solution of 2,4-dichlorobenzaldehyde (2.0 g, 11.4 mmol) in anhydrous THF (20 mL) was added diiodomethane (4.59 g, 17.1 mmol). The solution was cooled to 0° C. and butyl lithium-lithium bromide complex (1.5 M in Et₂O, 22.8 mmol) was added. The solution was stirred at 0° C. for 1 h and the solution was warmed to room temperature and allowed to stir an additional 1 h. To this reaction was slowly added ice. The mixture was then extracted with EtOAc (3×). The combined organic layers were dried over Na₂SO₄, filtered and concentrated to afford i (2.1 g) as an orange oil that was used without purification in Step 2.

### Step 2:

To a flask containing ii (prepared by the method of Synthesis (1992) 288-292; herein incorporated by reference) (1.55 g, 7.94 mmol) was added i (1.5 g, 7.94 mmol). The neat mixture was heated to 130° C. for 16 h to afford iii (3.0 g), which was used without purification in Step 3.

### Step 3:

To a solution of iii (1.5 g, 3.9 mmol) in DCE (10 mL) was added thionyl chloride (1.16 g, 9.8 mmol). The resultant solution was heated to reflux for 2 h. The reaction was slowly quenched with NaHCO₃ (aq.). The mixture was then extracted with CH₂Cl₂ (3×). The combined organic layers were dried over Na₂SO₄, filtered and concentrated to afford iv (1.64 g), which was used without purification in Step 4.

### Step 4:

To a solution of iv (1.64 g, 3.9 mmol) propylnitrite (20 mL) was added 4-chloroaniline (1.49 g, 11.7 mmol). The solution was heated to reflux overnight. The solution was partitioned between NaHCO₃ (aq.) and CH₂Cl₂. The aqueous layer was extracted with CH₂Cl₂ (3×). The combined organic layers were dried over Na₂SO₄, filtered, and concentrated. The crude product was purified by flash chromatography (SiO₂; gradient elution 100:0 to 9:1 hexanes:EtOAc) to afford Example 374 (265 mg) and after further purification by preparative TLC (SiO₂; 9:1 hexanes:EtOAc) Example 375 (60 mg). The products were converted to their HCl salts by dissolving in CH₂Cl₂ followed by the addition of 2N HCl (in ether). The solvent was then removed to provide the salts.

### Preparation of Examples 376-377

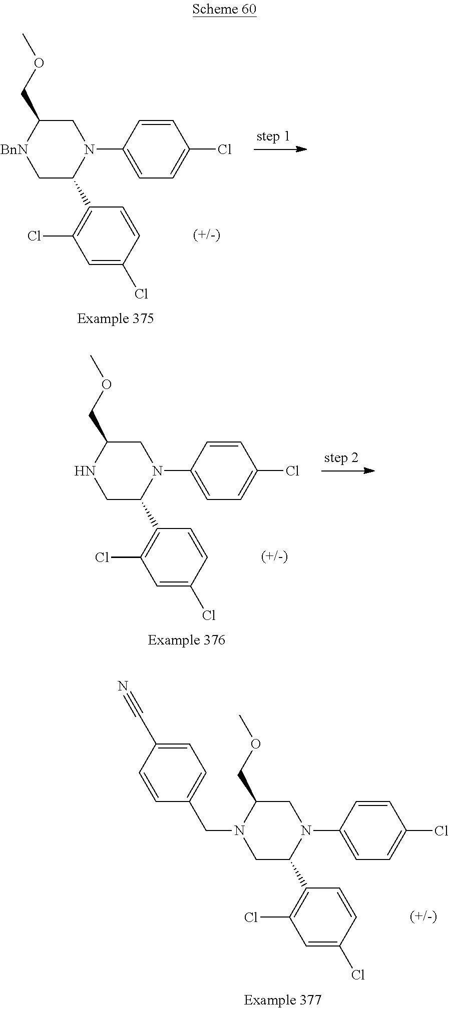 [SMILES: COC[C@H]1CN(c2ccc(Cl)cc2)[C@H](c2ccc(Cl)cc2Cl)CN1Cc1ccccc1 ; COC[C@H]1CN(c2ccc(Cl)cc2)[C@H](c2ccc(Cl)cc2Cl)CN1 ; COC[C@H]1CN(c2ccc(Cl)cc2)[C@H](c2ccc(Cl)cc2Cl)CN1Cc1ccc(C#N)cc1]

### Step 1:

To a solution of Example 375 (30 mg, 0.064 mmol) in DCE (2 mL) was added 1-chloroethyl chloroformate (10 mg, 0.07 mmol). The solution was heated to reflux for 1 h. Additional 1-chloroethyl chloroformate (8 mg, 0.056 mmol) was added and the solution was heated to reflux for an additional 8 h. The solution was concentrated. To the crude product was added MeOH (1 mL). The resultant solution was heated to reflux for 1.5 h. The solution was concentrated. The material was partitioned between CH₂Cl₂ and NaHCO₃ (aq.). The aqueous layer was extracted with CH₂Cl₂ (3×). The combined organic layers were dried over Na₂SO₄, filtered and concentrated. The crude product was purified by preparative TLC (SiO₂ 1:1 hexanes:EtOAc) to afford Example 376 (17 mg).

### Step 2:

To a solution of Example 376 (17 mg, 0.05 mmol) in CH₂Cl₂ (1 mL) was added 4-cyanobenzaldehyde (7 mg, 0.05 mmol) and sodium triacetoxyborohydride (16 mg, 0.075 mmol). The mixture was stirred at room temperature for 16 h. The mixture was partitioned between EtOAc and NaHCO₃ (aq.). The aqueous layer was extracted with EtOAc (3×). The combined organic layers were dried over Na₂SO₄, filtered and concentrated. The crude product was purified by preparative TLC (SiO₂ 4:1 hexanes:EtOAc) to afford Example 377. The product was converted to the HCl salt by dissolving in CH₂Cl₂ followed by the addition of 2N HCl (in ether). The solvent was then removed to provide the salt.

### Preparation of Example 378

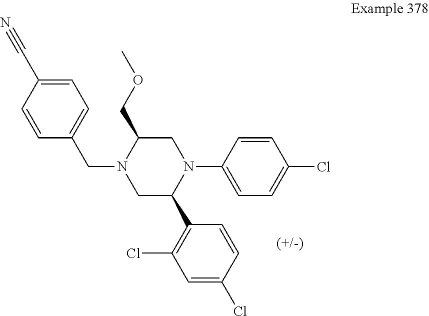 [SMILES: COC[C@H]1CN(c2ccc(Cl)cc2)[C@@H](c2ccc(Cl)cc2Cl)CN1Cc1ccc(C#N)cc1]

Example 378 was prepared using the same procedure used to prepare Example 377, except Example 374 was used as the starting material instead of Example 375.

### Preparation of Example 379

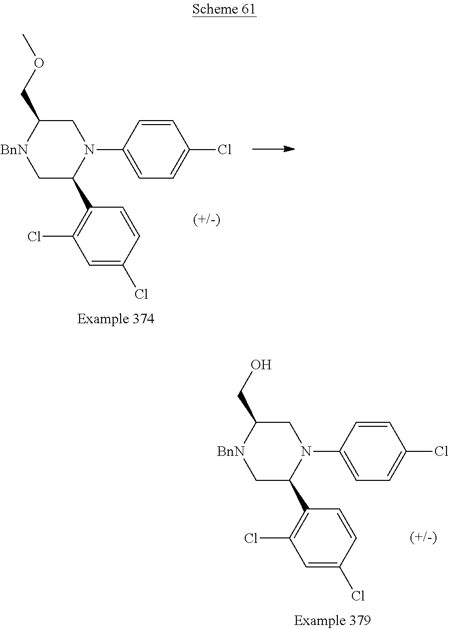 [SMILES: COC[C@H]1CN(c2ccc(Cl)cc2)[C@@H](c2ccc(Cl)cc2Cl)CN1Cc1ccccc1 ; OC[C@H]1CN(c2ccc(Cl)cc2)[C@@H](c2ccc(Cl)cc2Cl)CN1Cc1ccccc1]

To a solution of Example 374 (50 mg, 0.1 mmol) in CH₂Cl₂ (5 mL) at 0° C. was added BBr₃ (26 mg, 0.15 mmol). The solution was allowed to warm to room temperature and stirred for 3 h. To the solution was added NaHCO₃. The mixture was extracted with CH₂Cl₂ (3×). The combined organic layers were dried over Na₂SO₄, filtered and concentrated. The crude product was purified by preparative TLC (SiO₂ 4:1 hexanes:EtOAc) to afford Example 379. The product was converted to the HCl salt by dissolving in CH₂Cl₂ followed by the addition of 2N HCl (in ether). The solvent was then removed to provide the salt (9 mg).

### Preparation of Example 380

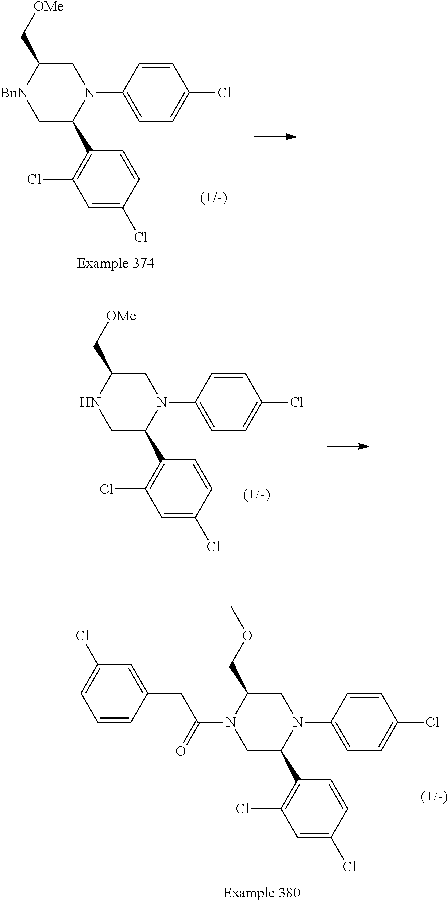 [SMILES: COC[C@H]1CN(c2ccc(Cl)cc2)[C@@H](c2ccc(Cl)cc2Cl)CN1Cc1ccccc1 ; COC[C@H]1CN(c2ccc(Cl)cc2)[C@@H](c2ccc(Cl)cc2Cl)CN1 ; COC[C@H]1CN(c2ccc(Cl)cc2)[C@@H](c2ccc(Cl)cc2Cl)CN1C(=O)Cc1cccc(Cl)c1]

### Step 1:

Example 374 was converted to the secondary amine using conditions similar to those used to prepare Example 376 in Scheme 60.

### Step 2:

The secondary amine prepared in step 1 was reacted with 3-chlorophenylacetic acid using conditions similar to those used to prepare Example 358 in Scheme 54.

### Preparation of Example 381

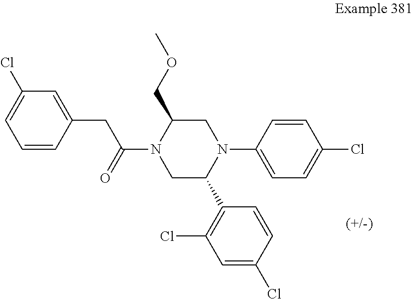 [SMILES: COC[C@H]1CN(c2ccc(Cl)cc2)[C@H](c2ccc(Cl)cc2Cl)CN1C(=O)Cc1cccc(Cl)c1]

Example 381 was prepared using conditions similar to those used to prepare Example 380, except that Example 376 was used instead of Example 374.

### Preparation of Example 382

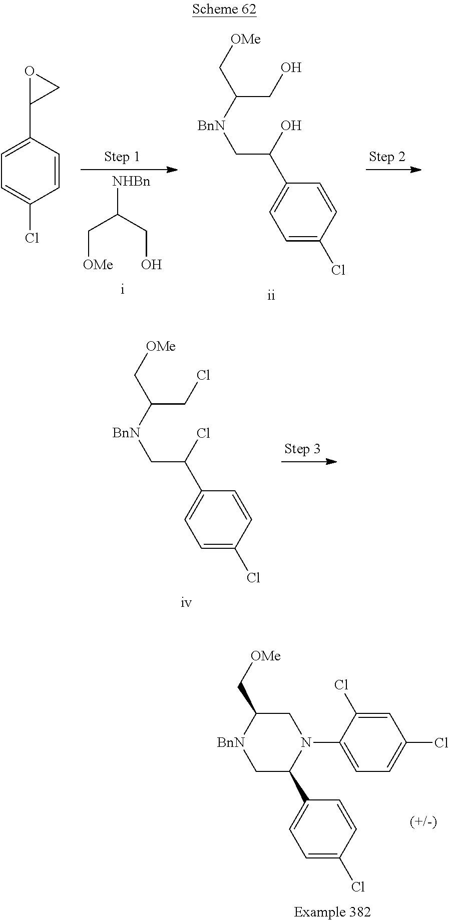

### Step 1:

To a flask containing i (1.0 g, 5.1 mmol) was added 4-chlorostyrene epoxide. The neat mixture was heated to 130° C. for 18 h to afford the diol ii which was used without purification.

### Step 2:

To a solution of ii (710 mg, 2.02 mmol) in CHCl₃ (15 mL) was added thionyl chloride (603 mg, 5.07 mmol). The resultant solution was heated to reflux for 3 h. The solution was cooled to room temperature and concentrated. The crude mixture was partitioned between CH₂Cl₂ and NaHCO₃ (aq.). The mixture was stirred vigorously for 10 min. The layers were separated and the aqueous layer was extracted with CH₂Cl₂ (3×). The combined organic layers were dried over Na₂SO₄, filtered and concentrated to afford iv, which was used without purification in Step 3.

### Step 3:

To a solution of the dichloride iv (3.0 mmol) in propionitrile (20 mL) was added 2,4-dichloroaniline (486 mg, 3.0 mmol) and iPr₂NEt (387 mg, 3.0 mmol). The mixture was heated to 100° C. for 16 h. Cooled solution to room temperature and concentrated.

The crude material was dissolved in anhydrous THF (10 mL). To this solution was added NaH (60 mg, 60% in oil). The mixture was heated to reflux for 16 h. Additional NaH (60 mg, 60% in oil) was added and the mixture was heated to reflux for an additional 24 h. Water was slowly added and the mixture was extracted with EtOAc (3×). The combined organic layers were washed with brine, dried over Na₂SO₄, filtered and concentrated. The crude product was purified by preparative TLC (SiO₂, 4:1 hexanes:EtOAc) to afford Example 382 (365 mg). The product was converted to the HCl salt by dissolving in CH₂Cl₂ followed by the addition of 2N HCl (in ether). The solvent was then removed to provide the salt.

### Preparation of Example 383

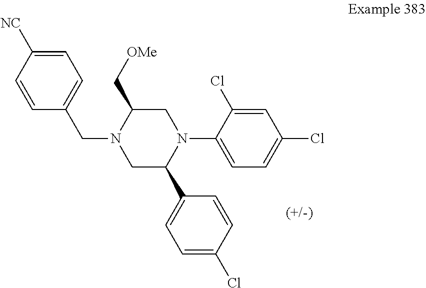 [SMILES: COC[C@H]1CN(c2ccc(Cl)cc2Cl)[C@@H](c2ccc(Cl)cc2)CN1Cc1ccc(C#N)cc1]

Example 383 was prepared using conditions similar to those used to prepare Example 377, except Example 374 was used as the starting material instead of Example 375.

### Preparation of Example 385

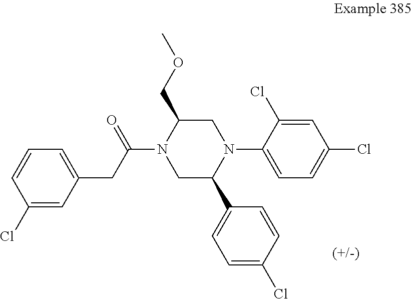 [SMILES: COC[C@H]1CN(c2ccc(Cl)cc2Cl)[C@@H](c2ccc(Cl)cc2)CN1C(=O)Cc1cccc(Cl)c1]

Example 385 was prepared using conditions similar to those used to prepare Example 380.

### Preparation of Examples 386-388

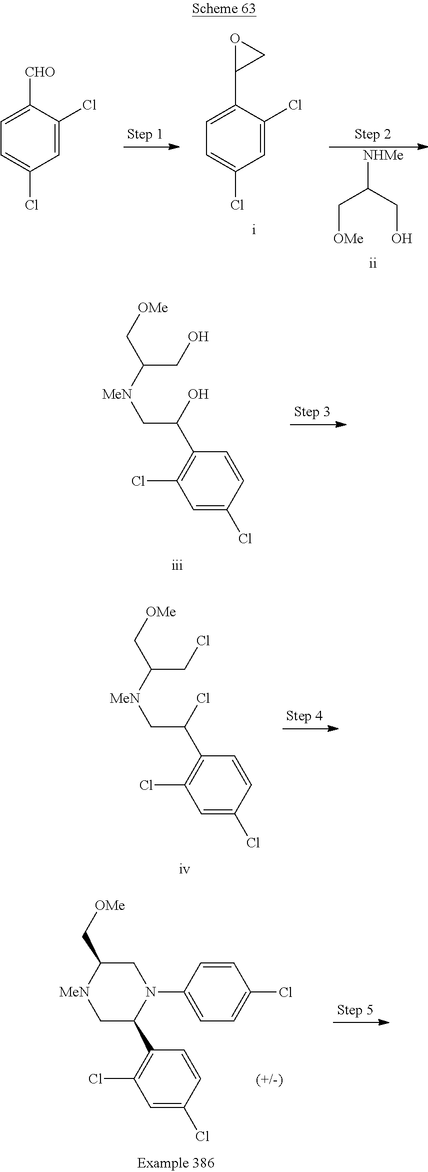 [SMILES: O=Cc1ccc(Cl)cc1Cl ; Clc1ccc(C2CO2)c(Cl)c1 ; CNC(CO)COC ; COCC(CO)N(C)CC(O)c1ccc(Cl)cc1Cl ; COCC(CCl)N(C)CC(Cl)c1ccc(Cl)cc1Cl ; COC[C@H]1CN(c2ccc(Cl)cc2)[C@@H](c2ccc(Cl)cc2Cl)CN1C]

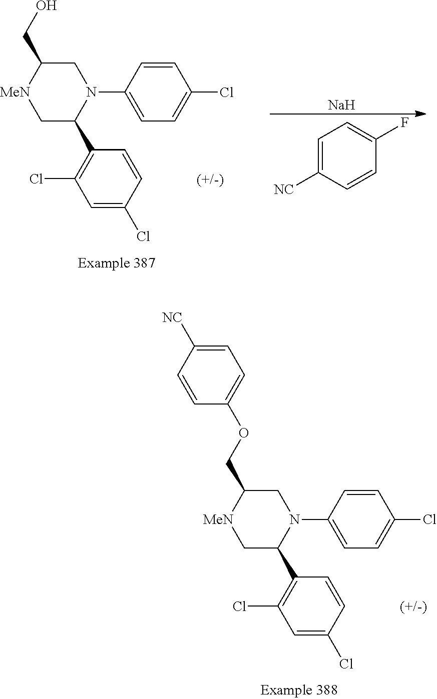

The N-methyl piperazine Example 386 was prepared using procedures similar to those described above for Example 374 (Steps 1, 2, 3, and 4). The alcohol Example 387 was prepared from the N-methyl piperazine Example 386 using a procedure similar to that used to prepare Example 379.

The alcohol Example 387 (30 mg) was taken up in DMF. Sodium hydride (12 mg of a 60 wt % dispersion in oil) was added. 4-Fluorobenzonitrile (35 mg) was added, and the solution was stirred at 25° C. (18 h). The solution was partitioned between EtOAc and water. The aqueous layer was extracted with EtOAc. The combined organic layers were washed with brine and dried (MgSO₄). Filtration and concentration gave a yellow oil. Purification via thin-layer preparative chromatography (2/1 hexanes/EtOAc, SiO₂) gave 21 mg (45%) of Example 388 as an oil.

### Preparation of Example 389

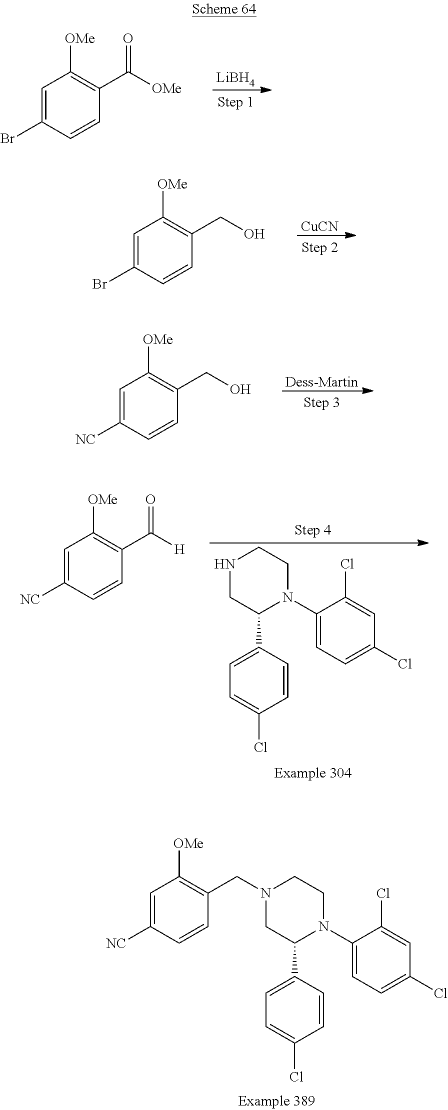

### Step 1:

To methyl-4-bromo-2-methoxybenzoate (Aldrich) (1.0 g, 4.1 mmol) in THF (10 mL), was added LiBH₄ (0.13 g, 6.1 mmol). Ethanol (2 mL) was added dropwise. The resulting reaction mixture was stirred at room temperature for 20 h. 1 N NaOH was added, and the mixture was extracted with EtOAc. The organic layers were combined and washed with water and brine, then dried (MgSO₄), filtered, and concentrated in vacuo to provide the corresponding benzyl alcohol (0.86 g, 4.0 mmol).

### Step 2:

To the benzyl alcohol prepared in step 1 (0.86 g, 4.0 mmol) in DMF (8 mL) was added CuCN (1.1 g, 12 mmol). The mixture was warmed to 150° C. and stirred for 20 h. The mixture was then cooled to room temperature and EtOAc was added, followed by a saturated NH₄Cl/NH₄OH solution. The mixture was stirred vigorously for 10 minutes and extracted with EtOAc, then the organic layer was dried (MgSO₄), filtered, and concentrated in vacuo. Purification by silica gel chromatography provided 4-hydroxymethyl-3-methoxy-benzonitrile (0.38 g, 2.3 mmol).

### Step 3:

To 4-hydroxymethyl-3-methoxy-benzonitrile prepared in step 2 (0.38 g, 2.3 mmol) in CH₂Cl₂ (8 mL) at room temperature was added Dess-Martin periodinane (1.2 g, 2.8 mmol). The mixture was stirred at room temperature for 20 h. The resulting white precipitate was filtered off and the filtrate concentrated in vacuo. The residue was purified by silica gel chromatography (20% EtOAc/Hex) to provide the corresponding aldehyde (0.32 g, 2.0 mmol).

### Step 4:

To the aldehyde prepared in step 3 (0.07 g, 0.44 mmol) in DCE (1 mL) was added the piperazine Example 304 (0.15 g, 0.44 mmol) followed by Na(OAc)₃BH (0.19 g, 0.88 mmol). The mixture was stirred at room temperature for 20 h. CH₂Cl₂ was added and the mixture was washed with 1 N NaOH, water and brine. The organic layer was dried (MgSO₄), filtered, and concentrated in vacuo. The residue was purified by silica gel chromatography (35% EtOAc/hexane) to provide Example 389 (0.21 g, 0.44 mmol).

### Preparation of Example 390

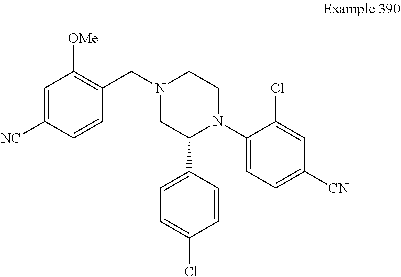 [SMILES: COc1cc(C#N)ccc1CN1CCN(c2ccc(C#N)cc2Cl)[C@H](c2ccc(Cl)cc2)C1]

Example 390 was prepared in a manner similar to that used to prepare Example 389 in Scheme 64, except that piperazine Example 305 was used instead of piperazine Example 304 in step 4.

### Preparation of Examples 391, 391a, and 391b

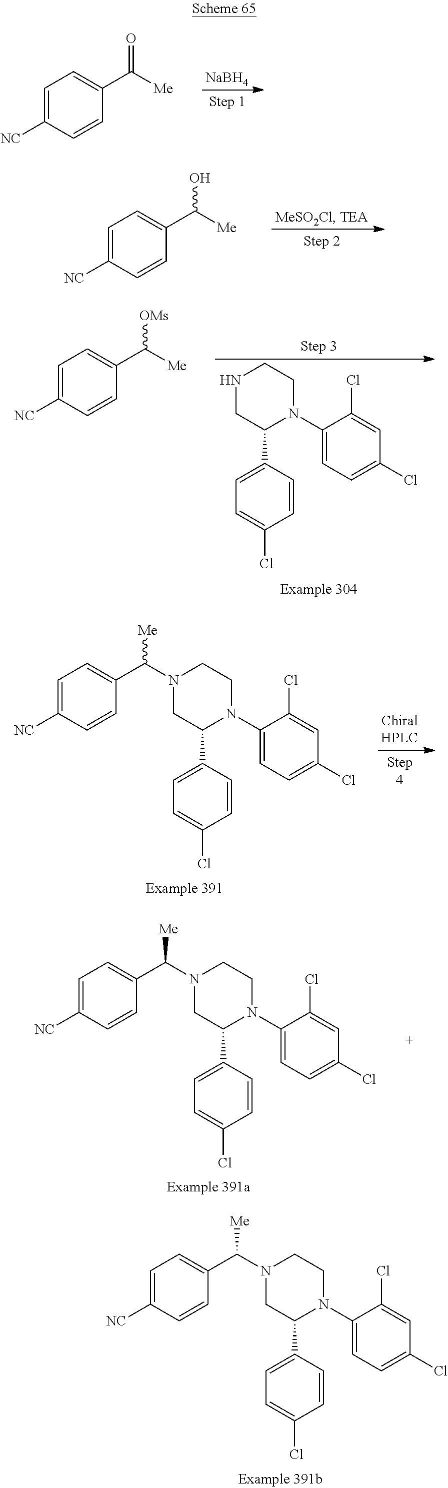

### Step 1:

To 4-acetylcyanobenzene (2.0 g, 13.8 mmol) in MeOH (55 mL) was added NaBH₄ (0.52 g, 13.0 mmol) in one portion. The reaction was stirred for 3 h, allowing the cold bath to warm. The reaction mixture was concentrated in vacuo. Water was added and the mixture was extracted with ether. The combined organic layers were washed with water and brine, dried (MgSO₄), filtered, and concentrated in vacuo to provide the corresponding alcohol (2.0 g, 13.6 mmol).

### Step 2:

To the alcohol prepared in step 1 (2.0 g, 13.6 mmol) in CH₂Cl₂ (45 mL) at 0° C. was added TEA (2.1 g, 20.4 mmol) followed by MeSO₂Cl (1.87 g, 16.3 mmol). The mixture was stirred for 20 h, allowing the cold bath to warm. CH₂Cl₂ was added and the combined organic layers were washed with saturated NaHCO₃, water, and brine, then dried (MgSO₄), filtered, and concentrated in vacuo to provide the corresponding mesylate (2.88 g, 12.8 mmol).

### Step 3:

To the mesylate prepared in step 2 (1.1 g, 4.8 mmol) in acetonitrile (13 mL) was added the piperazine Example 304 (1.3 g, 3.84 mmol) followed by K₂CO₃ (1.33 g, 9.6 mmol). The mixture was warmed to reflux and stirred for 20 h, cooled to room temperature, followed by the addition of water. The mixture was extracted with ethyl acetate, and the combined organic layers were washed with water and brine, dried (MgSO₄), filtered, and concentrated in vacuo. The concentrate was purified by silica gel chromatography (30% EtOAc/hexane) to provide Example 391 (1.44 g, 3.1 mmol) as a 1:1 mixture of diastereomers.

### Step 4:

The mixture of diastereomers of Example 391 were separated by preparative chiral HPLC (Chiralcel OD, 5×50 cm, 3% IPA/hexane, 48 ml/min, 254 nm) to provide a faster eluting and slower eluting stereoisomer.

### Preparation of Examples 392a and 392b

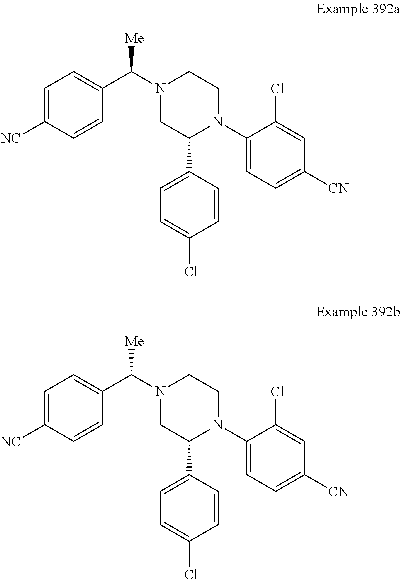 [SMILES: C[C@H](c1ccc(C#N)cc1)N1CCN(c2ccc(C#N)cc2Cl)[C@H](c2ccc(Cl)cc2)C1 ; C[C@@H](c1ccc(C#N)cc1)N1CCN(c2ccc(C#N)cc2Cl)[C@H](c2ccc(Cl)cc2)C1]

Examples 392a and 392b were prepared using procedures similar to those used to prepare Examples 391a and 391b in Scheme 65, above, except that the piperazine Example 305 was used in step 3 instead of Example 304. Examples 392a and 392b were separated by chiral preparative HPLC (Chiralcel OD, 5×50 cm, 10% IPA/hexane, 48 mL/min, 254 nm).

Alternatively, Example 392a was prepared by the following method:

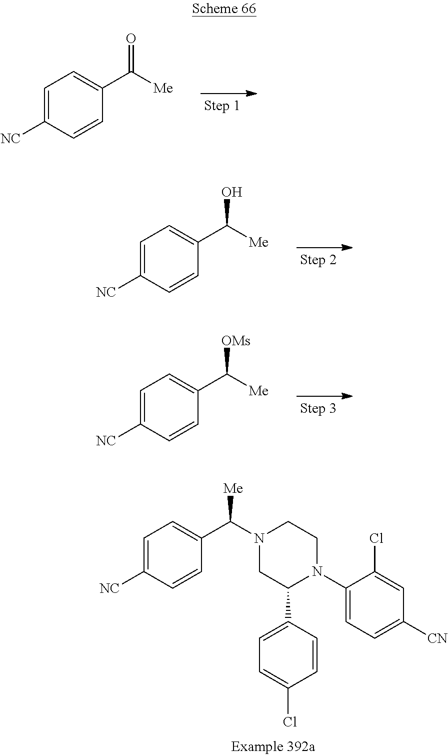 [SMILES: CC(=O)c1ccc(C#N)cc1 ; C[C@H](O)c1ccc(C#N)cc1 ; C[C@H](OS(C)(=O)=O)c1ccc(C#N)cc1 ; C[C@H](c1ccc(C#N)cc1)N1CCN(c2ccc(C#N)cc2Cl)[C@H](c2ccc(Cl)cc2)C1]

### Step 1:

To 4-acetylbenzonitrile (3.0 g, 20.7 mmol) in THF (21 mL) at −18° C. (CO₂/ethylene glycol bath) was added (R)-2-methyl-CBS-oxazaborolidine (1 M in toluene, 2.1 mL) followed by BH₃—SMe₂ (2.0M in THF, 7.2 mL) (following the chiral reduction procedure described in Chem. Rev., 1993, 93, 763-784). The cold bath was allowed to expire while stirring for 18 h. MeOH (−10 mL) was added (with gas evolution) and stirred for 15 minutes. The reaction mixture was concentrated in vacuo and taken up into EtOAc. The reaction mixture was then washed with 1N HCL, water, and brine. The organic layer was dried (MgSO₄), filtered, and concentrated in vacuo. The residue was purified by silica gel chromatography (5-40% EtOAc/hexanes) to provide the corresponding chiral alcohol (1.85 g, 12.6 mmol).

### Step 2:

To the alcohol prepared in step 1 (0.70 g, 4.8 mmol) in CH₂Cl₂ (16 mL) at 0° C. was added TEA (triethylamine; 0.72 g, 7.1 mmol) followed by methanesulfonyl chloride (0.60 g, 5.2 mmol). The reaction mixture was stirred at 0° C. for 1 h. CH₂Cl₂ was added and the mixture was washed with 1N HCL, water, and brine. The organic layer was dried (MgSO₄), filtered, and concentrated in vacuo to provide the corresponding chiral mesylate (1.1 g, 4.7 mmol) that was used directly in the next step without further purification.

### Step 3:

To the piperazine Example 305 (1.3 g, 4.0 mmol) in acetonitrile (13 mL) was added the mesylate prepared in step 2 (1.0 g, 4.6 mmol) followed by potassium carbonate (1.4 g, 10.1 mmol). The mixture was warmed to reflux and stirred for 36 h. The mixture was cooled to room temperature and water was added. The mixture was extracted with EtOAc. The organic layers were combined and washed with water and brine, dried (MgSO₄), filtered, and concentrated in vacuo. The resulting residue was purified by silica gel chromatography (0-10% MeOH/CH₂Cl₂) to provide a mixture of Example 392a and Example 392b (1.0 g) in ˜10:1 ratio as determined by chiral HPLC (Chiralcel OD, 10% IPA/hexanes, 1 mL/min, 254 nm—Example 392a retention time=12.0 min; Example 392b retention time 13.8 min). Example 392b was separated from Example 392a by preparative chiral HPLC (Chiralcel OD, 10% IPA/hexanes, 50 mL/min, 254 nm) to provide Example 392a (0.68 g, 1.48 mmol).

### Preparation of Examples 393a and 393b

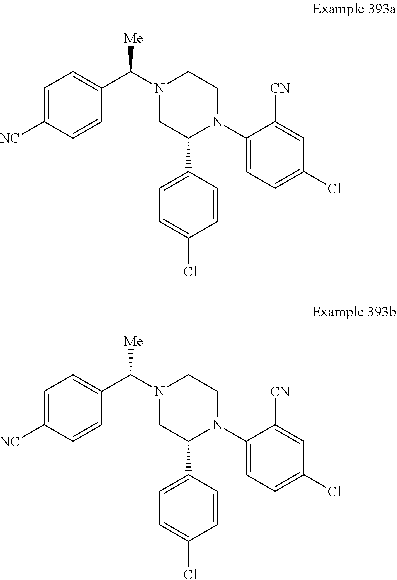 [SMILES: C[C@H](c1ccc(C#N)cc1)N1CCN(c2ccc(Cl)cc2C#N)[C@H](c2ccc(Cl)cc2)C1 ; C[C@@H](c1ccc(C#N)cc1)N1CCN(c2ccc(Cl)cc2C#N)[C@H](c2ccc(Cl)cc2)C1]

Examples 393a and 393b were prepared using procedures similar to those used to prepare Examples 391a and 391b in Scheme 65, above, except that piperazine Example 310 was used in step 3 instead of Example 304. Examples 393a and 393b were separated by chiral preparative HPLC (Chiralcel OD, 5×50 cm, 25% IPA/hexane, 50 mL/min., 254 nm).

### Preparation of Examples 394a and 394b

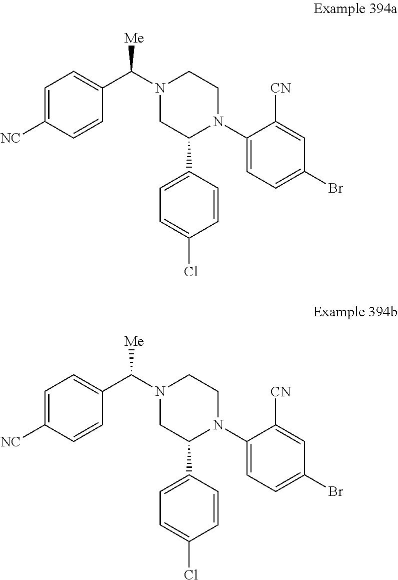 [SMILES: C[C@H](c1ccc(C#N)cc1)N1CCN(c2ccc(Br)cc2C#N)[C@H](c2ccc(Cl)cc2)C1 ; C[C@@H](c1ccc(C#N)cc1)N1CCN(c2ccc(Br)cc2C#N)[C@H](c2ccc(Cl)cc2)C1]

Examples 394a and 394b were prepared using procedures similar to those used to prepare Examples 391a and 391b in Scheme 65, above, except that piperazine Examples 306 was used in step 3 instead of Example 304. Examples 394a and 394b were separated by chiral preparative HPLC (Chiralcel OD, 5×50 cm, 5% IPA/hexane, 50 mL/min, 254 nm).

### Preparation of Example 395

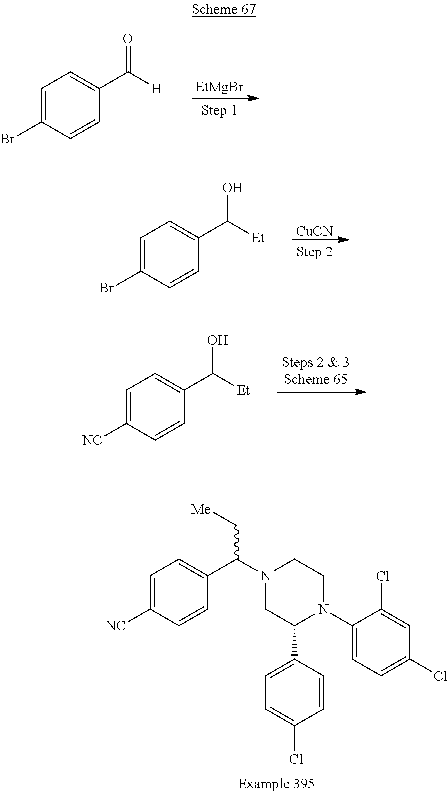

### Step 1:

To 4-bromobenzaldehyde (1.0 g, 5.4 mmol) in THF (18 mL) at 0° C. was added ethylmagnesium bromide (1.0 M in THF, 5.9 mL). The reaction mixture was stirred for 40 minutes. Water was added, then 25% aqueous sodium citrate solution. The mixture was extracted with EtOAc. The organic layers were combined and washed with water and brine. The organic layer was dried (MgSO₄), filtered, and concentrated in vacuo. Purification by silica gel chromatography (20% EtOAc/hexane) provided the corresponding alcohol (0.81 g, 3.8 mmol).

### Step 2:

To the alcohol prepared in step 1 (0.8 g, 3.8 mmol) in DMF (14 mL) was added CuCN (1.16 g, 12.9 mmol). The reaction mixture was warmed the to 150° C., stirred for 18 h and then cooled to room temperature. A NH₄Cl/saturated NH₄OH (9:1) solution was then added and the mixture was extracted with EtOAc. The organic layers were combined and dried (MgSO₄), filtered, and concentrated in vacuo. Purification by silica gel chromatography (30% EtOAc/hexane) provided the corresponding nitrile (0.34 g, 2.1 mmol). The nitrile was converted to Example 395 the procedures described above in steps 2 and 3 of Scheme 65.

### Preparation of Example 396

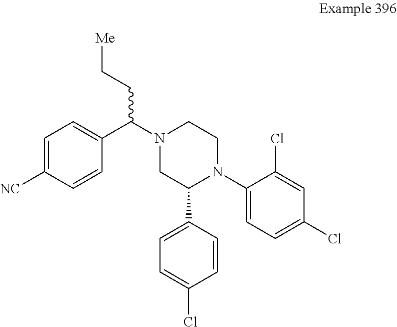 [SMILES: CCCC(c1ccc(C#N)cc1)N1CCN(c2ccc(Cl)cc2Cl)[C@H](c2ccc(Cl)cc2)C1]

The propyl piperazine Example 396 was prepared using the procedures of Scheme 67 except that propylmagnesium chloride was used instead of ethylmagnesium bromide in step 1.

### Preparation of Example 397

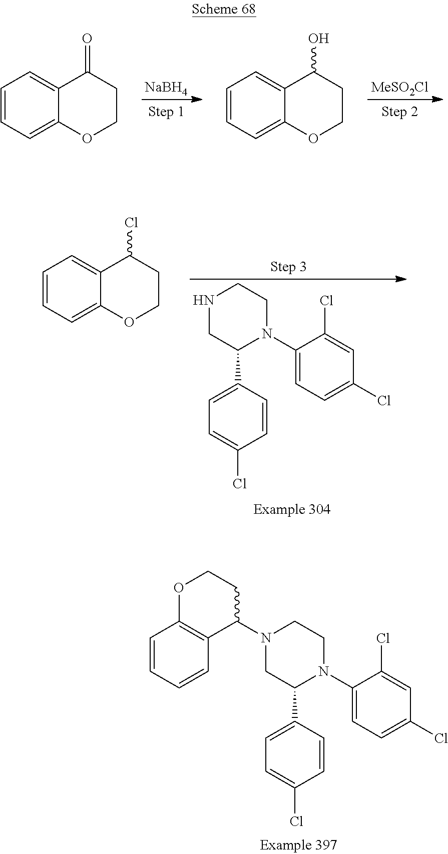

### Step 1:

To 4-chromanone (1.0 g, 6.75 mmol) in MeOH (20 mL) was added NaBH₄ (0.51 g, 13.5 mmol). The reaction mixture was stirred for 2 h and then concentrated in vacuo. 1N HCl was added and the mixture was extracted with EtOAc. The organic layers were combined and washed with water and brine. The organic layer was dried (MgSO₄), filtered, and concentrated in vacuo to provide the corresponding alcohol (1.01 g, 6.75 mmol).

### Step 2:

To the alcohol prepared in step 1 (1.1 g, 7.3 mmol) in CH₂Cl₂ (20 mL) at 0° C. was added TEA (1.53 mL, 11 mmol) followed by methanesulfonyl chloride (0.68 mL, 8.8 mmol). The reaction mixture was stirred for 1 h and CH₂Cl₂ was added. The mixture was washed with water and brine. The organic layer was dried (MgSO₄), filtered, and concentrated in vacuo to provide the corresponding chloride (1.23 g, 5.38 mmol).

### Step 3:

To the piperazine Example 304 (0.10 g, 0.29 mmol) in acetonitrile (1 mL) was added the chloride prepared in step 2 (0.06 g, 0.37 mmol) followed by K₂CO₃ (0.10 g, 0.73 mmol). The mixture was warmed to reflux and stirred for 20 h. The reaction mixture was concentrated in vacuo and EtOAc was added. The mixture was washed with water and brine, dried (MgSO₄), filtered, and concentrated in vacuo. The residue was purified by silica gel preparative TLC (2000 μm, 10% EtOAc/hexane) to provide Example 397 (0.80 g, 0.17 mmol).

### Preparation of Example 398

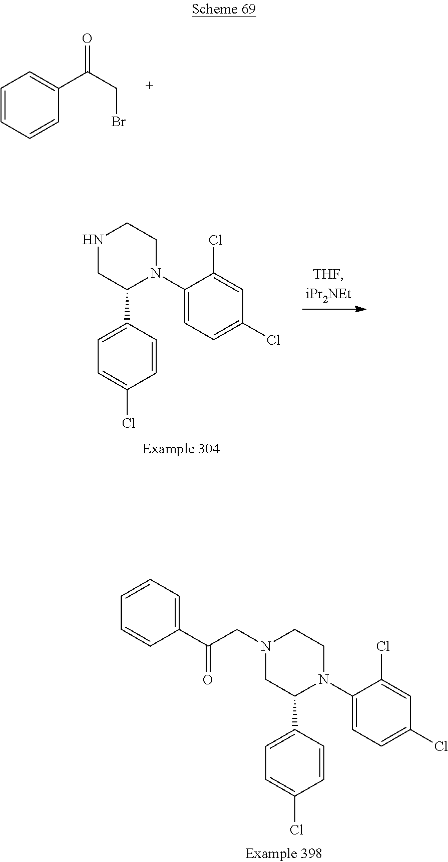 [SMILES: O=C(CBr)c1ccccc1 ; Clc1ccc([C@@H]2CNCCN2c2ccc(Cl)cc2Cl)cc1 ; O=C(CN1CCN(c2ccc(Cl)cc2Cl)[C@H](c2ccc(Cl)cc2)C1)c1ccccc1]

To the piperazine Example 304 (1.0 g, 2.9 mmol) in THF (10 mL) was added diisopropylethyl amine (1.1 g, 8.7 mmol) followed by 2-bromoacetophenone (1.1 g, 5.8 mmol). The mixture was stirred for 75 minutes at room temperature and water was added. The mixture was extracted with ethyl acetate, then the combined organic layers were washed with water and brine, dried (MgSO₄), filtered, and concentrated in vacuo. Purification by silica gel chromatography (30% EtOAc/hexane) provided Example 398 (1.1 g, 2.4 mmol).

### Preparation of Example 399

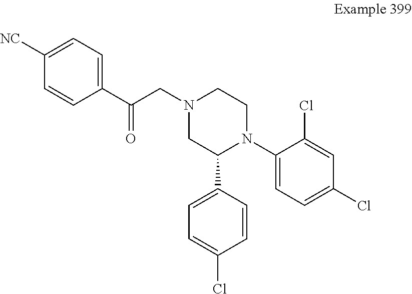 [SMILES: N#Cc1ccc(C(=O)CN2CCN(c3ccc(Cl)cc3Cl)[C@H](c3ccc(Cl)cc3)C2)cc1]

Example 399 was prepared using procedures similar to those used to prepare Example 398 in Scheme 69 except that 2-bromo-4′-cyanoacetophenone was used instead of 2-bromoacetophenone.

### Preparation of Example 400

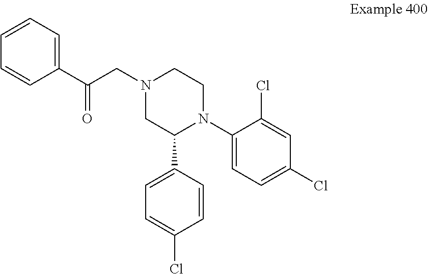 [SMILES: O=C(CN1CCN(c2ccc(Cl)cc2Cl)[C@H](c2ccc(Cl)cc2)C1)c1ccccc1]

Example 400 was prepared using procedures similar to those used to prepare Example 398 in Scheme 69 except that the racemic piperazine Example 1 was used instead of chiral piperazine Example 304.

### Preparation of Examples 401-410

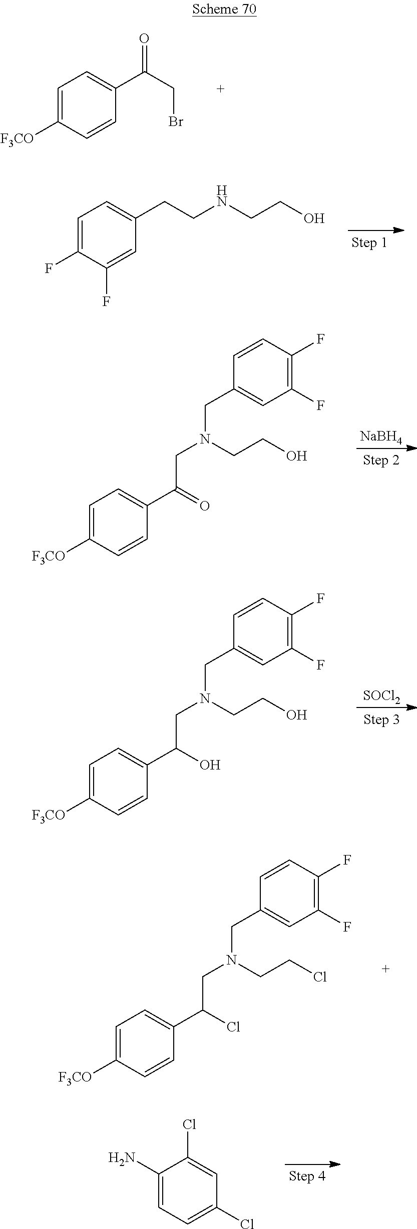

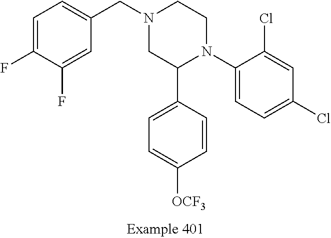 [SMILES: Fc1ccc(CN2CCN(c3ccc(Cl)cc3Cl)C(c3ccc(OC(F)(F)F)cc3)C2)cc1F]

### Step 1:

To the 2-[2-(3,4-difluorophenyl)-ethylamino]-ethanol (0.5 g, 2.7 mmol) in acetonitrile (5 mL) was added 2-bromo-1-(4-trifluoromethoxyphenyl)-ethanone (0.76 g, 2.7 mmol) and K₂CO₃ (0.44 g, 3.2 mmol). The reaction mixture was stirred at room temperature for 20 h, and concentrated in vacuo. Water was added to the concentrate, which was then extracted with EtOAc. The combined organic layers were washed with water and brine, dried (MgSO₄), filtered, and concentrated in vacuo. Purification by silica gel chromatography (40% EtOAc/hexane) provided 2-[(3,4-difluorobenzyl)-(2-hydroxyethyl)-amino]-1-(4-trifluoromethoxyphenyl)-ethanone (0.8 g, 2.1 mmol).

### Step 2:

To 2-[(3,4-difluorobenzyl)-(2-hydroxyethyl)-amino]-1-(4-trifluoromethoxyphenyl)-ethanone (0.8 g, 2.1 mmol) in MeOH (10 mL) at 0° C. was added NaBH₄ (0.12 g, 3.1 mmol) in one portion. The mixture was allowed to warm to room temperature and stirred for 20 h. The reaction mixture was concentrated in vacuo, and the residue was taken up into CH₂Cl₂ and washed with 1N NaOH, water, and brine. The organic layer was dried (MgSO₄), filtered, and concentrated in vacuo to provide a diol (0.8 g, 2.1 mmol).

### Step 3:

To the diol prepared in step 2 (0.8 g, 2.1 mmol) in CHCl₃ (16 mL) at 0° C. was added thionyl chloride (4 mL). The reaction mixture was allowed to warm to room temperature and then warmed to reflux and stirred for 2 h. The reaction was cooled to room temperature and concentrated in vacuo. The residue was taked up into CH₂Cl₂ and stirred vigorously with saturated NaHCO_{3(aq)}. The organic layer was washed with water and brine, then dried (MgSO₄), filtered, and concentrated in vacuo to provide the corresponding dichloride (0.74 g).

### Step 4:

To the dichloride prepared in step 3 (0.15 g, 0.35 mmol) in propionitrile (1.5 mL) was added the aniline (0.17 g, 1.1 mmol). The reaction mixture was warmed to reflux and stirred for 20 h. The reaction was then concentrated in vacuo. The residue was taken up into CH₂Cl₂ and washed with saturated NaHCO_{3(aq)}, water, and brine. The organic layer was dried (MgSO₄), filtered, and concentrated in vacuo. The residue was purified by preparative silica TLC (25% EtOAc/hexane) to provide Example 401 (0.5 g).

Examples 402-410 listed below in Table XIII were prepared from the appropriate bromoketone and substituted aniline using the general procedure described in Scheme 70.

TABLE XIII

| Example # | Bromoketone | Aniline | Example Structure |
|---|---|---|---|
| 402 | 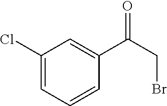 [SMILES: O=C(CBr)c1cccc(Cl)c1] | 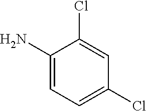 | 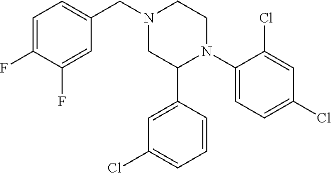 [SMILES: Fc1ccc(CN2CCN(c3ccc(Cl)cc3Cl)C(c3cccc(Cl)c3)C2)cc1F] |
| 403 | 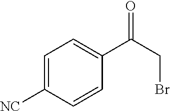 [SMILES: N#Cc1ccc(C(=O)CBr)cc1] | 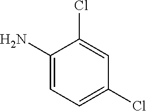 | 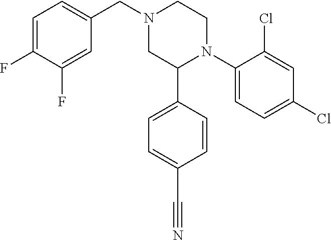 [SMILES: N#Cc1ccc(C2CN(Cc3ccc(F)c(F)c3)CCN2c2ccc(Cl)cc2Cl)cc1] |
| 404 | 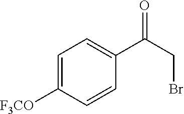 [SMILES: O=C(CBr)c1ccc(OC(F)(F)F)cc1] | 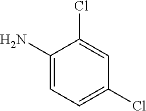 | 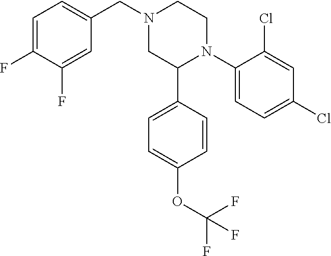 [SMILES: Fc1ccc(CN2CCN(c3ccc(Cl)cc3Cl)C(c3ccc(OC(F)(F)F)cc3)C2)cc1F] |
| 405 | 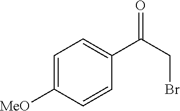 [SMILES: COc1ccc(C(=O)CBr)cc1] | 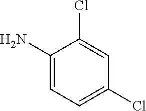 | 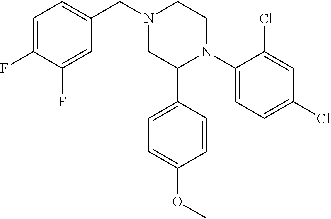 [SMILES: COc1ccc(C2CN(Cc3ccc(F)c(F)c3)CCN2c2ccc(Cl)cc2Cl)cc1] |
| 406 | 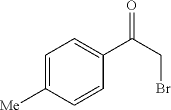 [SMILES: Cc1ccc(C(=O)CBr)cc1] | 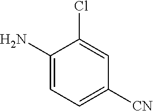 | 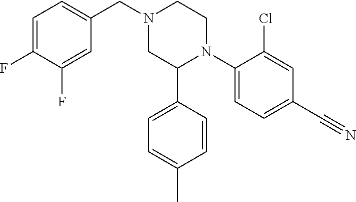 [SMILES: Cc1ccc(C2CN(Cc3ccc(F)c(F)c3)CCN2c2ccc(C#N)cc2Cl)cc1] |
| 407 | 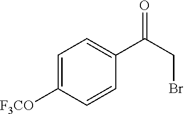 [SMILES: O=C(CBr)c1ccc(OC(F)(F)F)cc1] | 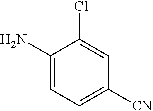 | 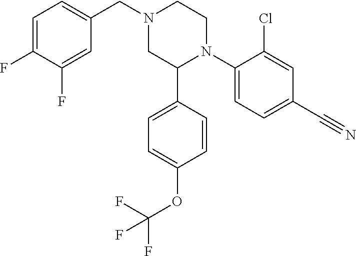 [SMILES: N#Cc1ccc(N2CCN(Cc3ccc(F)c(F)c3)CC2c2ccc(OC(F)(F)F)cc2)c(Cl)c1] |
| 408 | 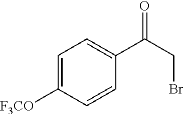 [SMILES: O=C(CBr)c1ccc(OC(F)(F)F)cc1] | 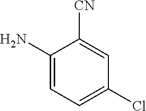 | 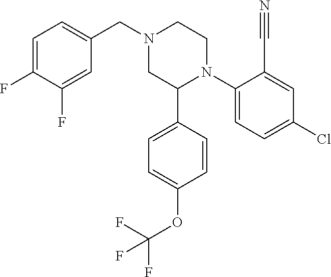 [SMILES: N#Cc1cc(Cl)ccc1N1CCN(Cc2ccc(F)c(F)c2)CC1c1ccc(OC(F)(F)F)cc1] |
| 409 | 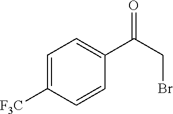 [SMILES: O=C(CBr)c1ccc(C(F)(F)F)cc1] | 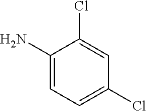 | 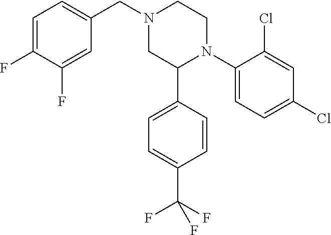 [SMILES: Fc1ccc(CN2CCN(c3ccc(Cl)cc3Cl)C(c3ccc(C(F)(F)F)cc3)C2)cc1F] |
| 410 | 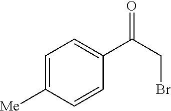 [SMILES: Cc1ccc(C(=O)CBr)cc1] | 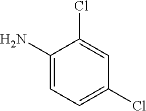 | 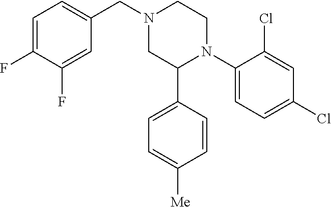 [SMILES: Cc1ccc(C2CN(Cc3ccc(F)c(F)c3)CCN2c2ccc(Cl)cc2Cl)cc1] |

### Preparation of Example 411

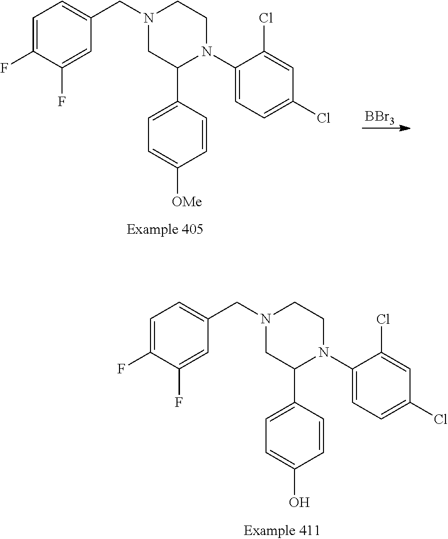

To the piperazine Example 405 (0.125 g, 0.27 mmol) in CH₂Cl₂ (1 mL) at −78° C. was added boron tribromide (1.0 M in CH₂Cl₂, 0.3 mL). The cold bath was removed from the reaction vessel and the reaction mixture was stirred for 30 minutes. Additional boron tribromide (0.6 mL) was added and the reaction was stirred at room temperature for 20 h. The reaction mixture was diluted with CH₂Cl₂ and poured into cold saturated NaHCO_{3(aq)}. The mixture was extracted with CH₂Cl₂. The organic layers were combined and washed with water and brine. The organic layer was then dried (MgSO₄), filtered, and concentrated in vacuo. The residue was triturated with ether to provide Example 411 (0.036 g).

### Preparation of Examples 412-415

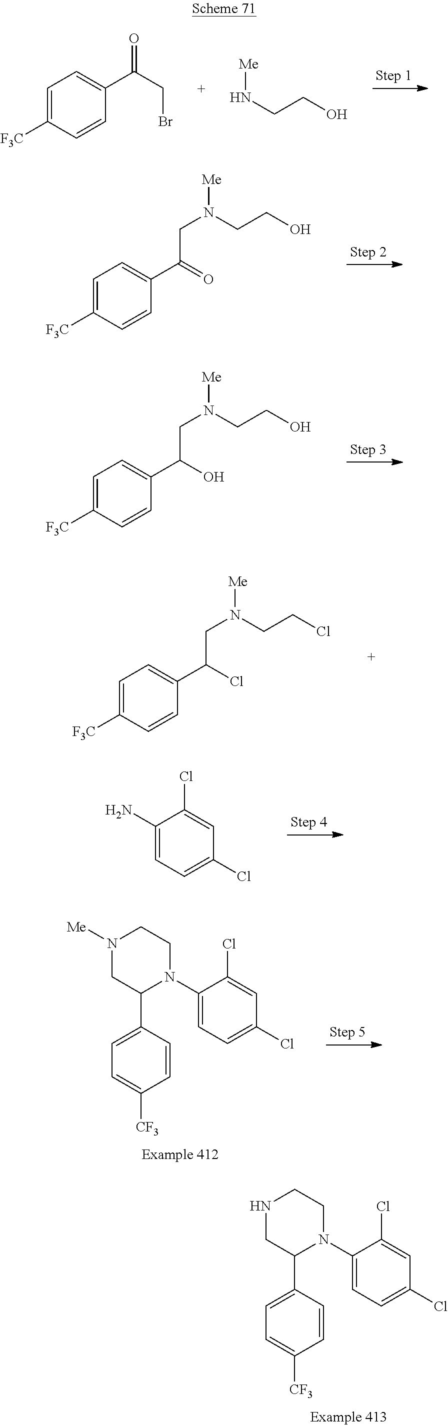

### Step 1:

To the bromoketone (1.0 g, 3.6 mmol) in acetonitrile (7 mL) was added 2-(methylamino)ethanol (0.3 mL) and K₂CO₃ (0.6 g, 4.35 mmol). The reaction mixture was stirred at room temperature for 2 h. The reaction mixture was concentrated in vacuo. The resulting residue was taken up into EtOAc and then washed with water and brine. The organic layer was dried (MgSO₄), filtered, and concentrated in vacuo to provide an amino alcohol (0.85 g) which was used in step 2 without further purification.

### Step 2:

To the amino alcohol prepared in step 1 (0.83 g, 3.2 mmol) in MeOH (10 mL) at 0° C. was added NaBH₄ (0.18 g, 4.8 mmol) in one portion. The reaction mixture was allowed to warm to room temperature while stirring for 20 h. The reaction was concentrated in vacuo and taken up into CH₂Cl₂ and washed with 1N NaOH, water, and brine. The organic layer was dried (MgSO₄), filtered, and concentrated in vacuo to provide the corresponding diol (0.8 g).

### Step 3:

To the diol prepared in step 2 (0.8 g, 3.0 mmol) in dichloroethane (24 mL) at 0° C. was added thionyl chloride (6 mL). The reaction mixture was allowed to warm to room temperature and then heated to reflux for 2 h. The reaction mixture was concentrated in vacuo, taken up into CH₂Cl₂ and stirred vigorously with saturated NaHCO_{3(aq)}. The organic layer was washed with water and brine and dried (MgSO₄). The organic layer was filtered and concentrated in vacuo to provide the corresponding dichloride (0.74 g).

### Step 4:

To the dichloride prepared in step 3 (0.2 g, 0.67 mmol) in propionitrile (2 mL) was added 2,4-dichloroaniline (0.32 g). The reaction mixture was warmed to reflux and stirred for 20 h. The reaction was concentrated in vacuo and the residue taken up into CH₂Cl₂. The reaction mixture was then washed with 1N NaOH, water, and brine. The organic layer was dried (MgSO₄), filtered, and concentrated in vacuo to provide a residue that was purified by silica gel chromatography (0-8% MeOH/EtOAc) to provide Example 412 (0.24 g).

### Step 5:

To the piperazine Example 412 (0.24 g) in dichloroethane (2 mL) was added proton sponge (0.04 g) and 1-chloroethylchloroformate (0.13 mL). The reaction mixture was warmed to reflux and stirred for 20 h. The reaction mixture was concentrated in vacuo and the residue taken up into MeOH (2 mL). The soution was warmed to reflux and stirred for 3 h. The reaction mixture was concentrated in vacuo, the resulting residue taken up into CH₂Cl₂, and then washed with 1N NaOH, water, and brine. The organic layer was dried (MgSO₄), filtered, and concentrated in vacuo. The residue was purified by silica gel chromatography (8% MeOH/CH₂Cl₂) to provide the piperazine Example 413 (0.2 M.

The following piperazines were prepared as in Scheme 71 above using the α-bromoketone listed in Table XIV in step 1.

TABLE XIV

| Example # | α-Bromoketone | Example Structure |
|---|---|---|
| 414 | 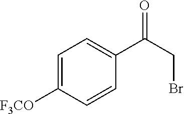 [SMILES: O=C(CBr)c1ccc(OC(F)(F)F)cc1] | 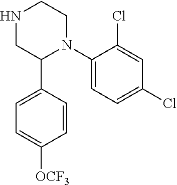 [SMILES: FC(F)(F)Oc1ccc(C2CNCCN2c2ccc(Cl)cc2Cl)cc1] |
| 415 | 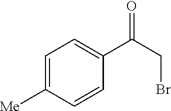 [SMILES: Cc1ccc(C(=O)CBr)cc1] | 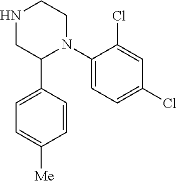 [SMILES: Cc1ccc(C2CNCCN2c2ccc(Cl)cc2Cl)cc1] |

### Preparation of Example 416

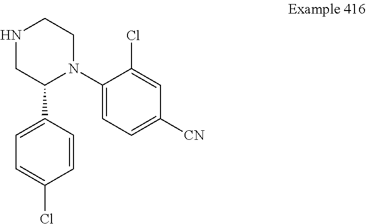 [SMILES: N#Cc1ccc(N2CCNC[C@H]2c2ccc(Cl)cc2)c(Cl)c1]

The piperazine Example 416 was prepared in a manner similar to that used to prepare Example 1 in Scheme 1 except that 4-amino-3-chlorobenzonitrile was used instead of 2,4-dichloroaniline in step 3.

### Preparation of Example 417

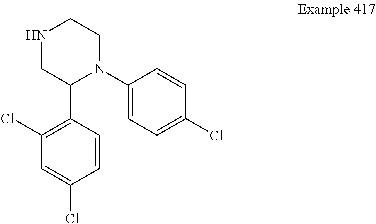 [SMILES: Clc1ccc(N2CCNCC2c2ccc(Cl)cc2Cl)cc1]

Piperazine Example 417 was prepared in the same manner as Example 1 in Scheme 1 except that 2-(2,4-dichlorophenyl)oxirane (prepared from 2,4-dichlorobenzaldehyde as in step 1 of scheme 7) was used instead of 2-(4-chlorophenyl)oxirane in step 1 and 4-chloroaniline was used instead of 2,4-dichloroaniline in step 3.

### Preparation of Example 418

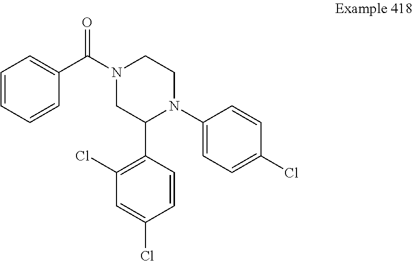 [SMILES: O=C(c1ccccc1)N1CCN(c2ccc(Cl)cc2)C(c2ccc(Cl)cc2Cl)C1]

Example 418 was prepared in the same manner as Example 4 in Scheme 2 except that the piperazine Example 417 was used instead of Example 1.

### Preparation of Example 419

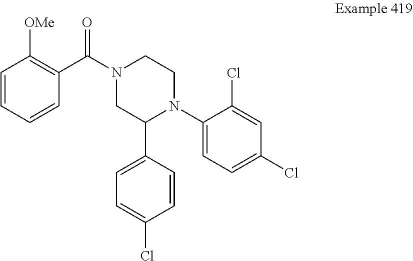 [SMILES: COc1ccccc1C(=O)N1CCN(c2ccc(Cl)cc2Cl)C(c2ccc(Cl)cc2)C1]

Example 419 was prepared in the same manner as Example 4 in Scheme 2 except that 2-methoxybenzoyl chloride was used instead of benzoyl chloride.

### Preparation of Example 420

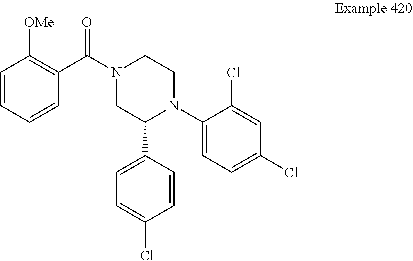 [SMILES: COc1ccccc1C(=O)N1CCN(c2ccc(Cl)cc2Cl)[C@H](c2ccc(Cl)cc2)C1]

Example 420 was prepared in the same manner as Example 4 in Scheme 2 except that the piperazine Example 304 was used instead of Example 1 and 2-methoxybenzoyl chloride was used instead of benzoyl chloride.

### Preparation of Examples 421-424

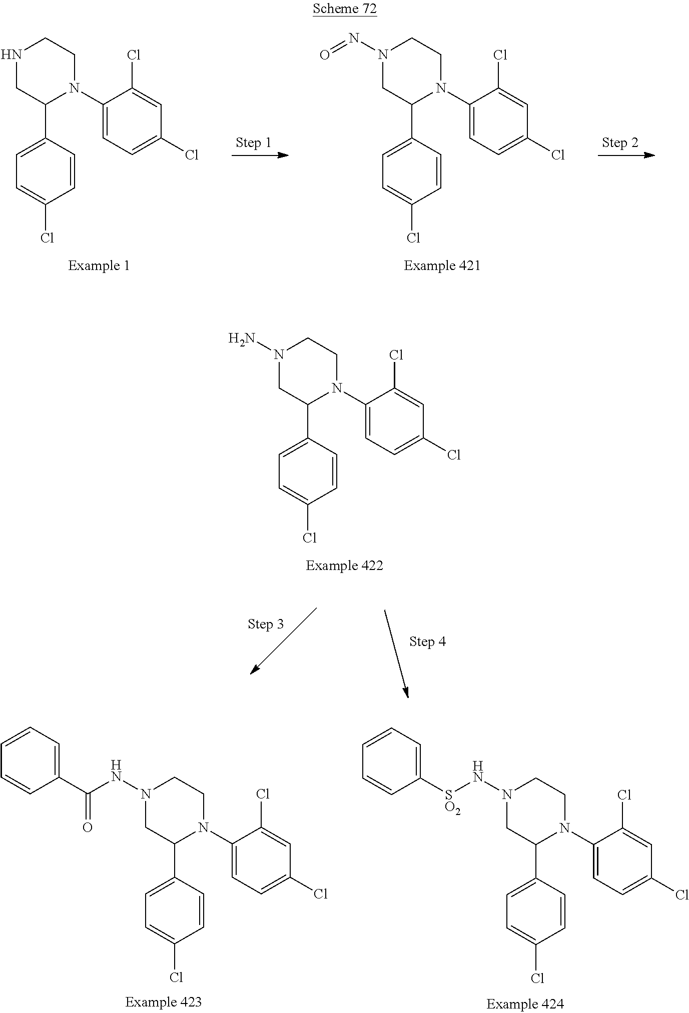 [SMILES: Clc1ccc(C2CNCCN2c2ccc(Cl)cc2Cl)cc1 ; O=NN1CCN(c2ccc(Cl)cc2Cl)C(c2ccc(Cl)cc2)C1 ; NN1CCN(c2ccc(Cl)cc2Cl)C(c2ccc(Cl)cc2)C1 ; O=C(NN1CCN(c2ccc(Cl)cc2Cl)C(c2ccc(Cl)cc2)C1)c1ccccc1 ; O=S(=O)(NN1CCN(c2ccc(Cl)cc2Cl)C(c2ccc(Cl)cc2)C1)c1ccccc1]

### Step 1:

To the piperazine Example 1 (0.31 g, 0.89 mmol) in THF (3 mL) at 0° C. was added 3N HCl_{(aq)} (1.5 mL). NaNO₂ (0.14 g, 2.1 mmol) in water (0.9 mL) was then added and the cold bath was removed from the reaction vessel. The reaction mixture was stirred for 15 h. The reaction mixture was then made basic with 3N NaOH, extracted with EtOAc, and the organic layer was washed with water and brine. The organic layer was then dried (MgSO₄), filtered, and concentrated in vacuo to provide Example 421 (0.31 g, 085 mmol).

### Step 2:

To the piperazine Example 421 (0.31 g, 0.85 mmol) in AcOH (3 mL) was added water (1.4 mL). Zinc dust was added (0.062 g) in one portion. THF (1 mL) was then added, and the reaction mixture was warmed to 50° C. After 1 h, additional zinc dust (0.3 g) was added. The reaction mixture was stirred at reflux for an additional 1 h. The reaction mixture was cooled to room temperature and filtered. EtOAc was added and the mixture was washed with 3N NaOH, water, and brine. The organic layer was dried (MgSO₄), filtered, and concentrated in vacuo. The residue was purified by silica gel chromatography (6% MeOH/CH₂Cl₂) to provide Example 422 (0.17 g).

### Step 3:

To the aminopiperazine Example 422 (0.07 g, 0.16 mmol) in dichloroethane (1 mL) was added TEA (0.05 g, 0.48 mmol) and benzoyl chloride (0.03 g, 0.2 mmol). The reaction mixture was stirred at room temperature for 1 h. CH₂Cl₂ was added to the reaction mixture, which was then washed with saturated NaHCO₃, water, and brine. The mixture was then dried (MgSO₄), filtered, and the organic layer was concentrated in vacuo. The concentrate was purified by preparative TLC (SiO₂) (50% EtOAc/hexane) to provide Example 423 (0.07 g).

### Step 4:

To the piperazine Example 422 (0.074 g, 0.17 mmol) in dichloroethane (1 mL) was added TEA (0.05 g, 0.5 mmol) followed by benzene sulfonyl chloride (0.04 g). The mixture was stirred at room temperature for 2 h and CH₂Cl₂ was added. The mixture was washed with saturated NaHCO₃, water, and brine, dried (MgSO₄), filtered, and the organic layer was concentrated in vacuo. Purification by preparative TLC (SiO₂) (20% EtOAc/hexane) provided Example 424 (0.05 g).

### Preparation of Example 425

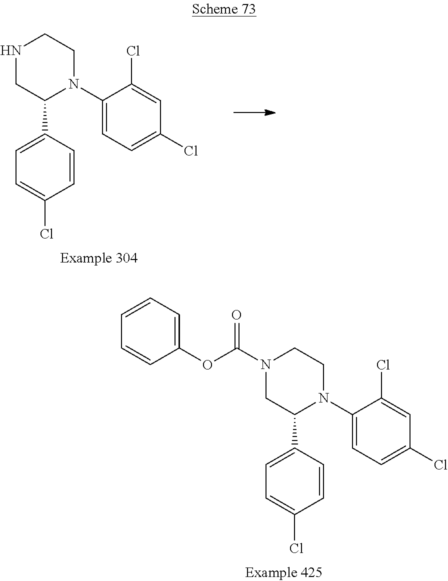 [SMILES: Clc1ccc([C@@H]2CNCCN2c2ccc(Cl)cc2Cl)cc1 ; O=C(Oc1ccccc1)N1CCN(c2ccc(Cl)cc2Cl)[C@H](c2ccc(Cl)cc2)C1]

The carbamate Example 425 was prepared in the same manner as Example 4 in Scheme 2 except that Example 304 was used as a starting material instead of Example 1 and phenylchloroformate was used instead of benzoyl chloride.

### Preparation of Example 426

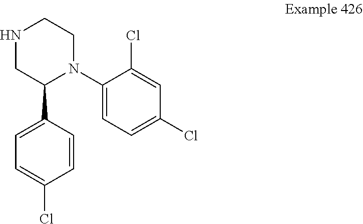 [SMILES: Clc1ccc([C@H]2CNCCN2c2ccc(Cl)cc2Cl)cc1]

The piperazine Example 426 was prepared in the same manner as the chiral piperazine Example 304 in Scheme 28 except that (S)-2-methyl-CBS-oxazaborolidine was used instead of (R)-2-methyl-CBS-oxazaborolidine in step 1.

### Preparation of Examples 427-428

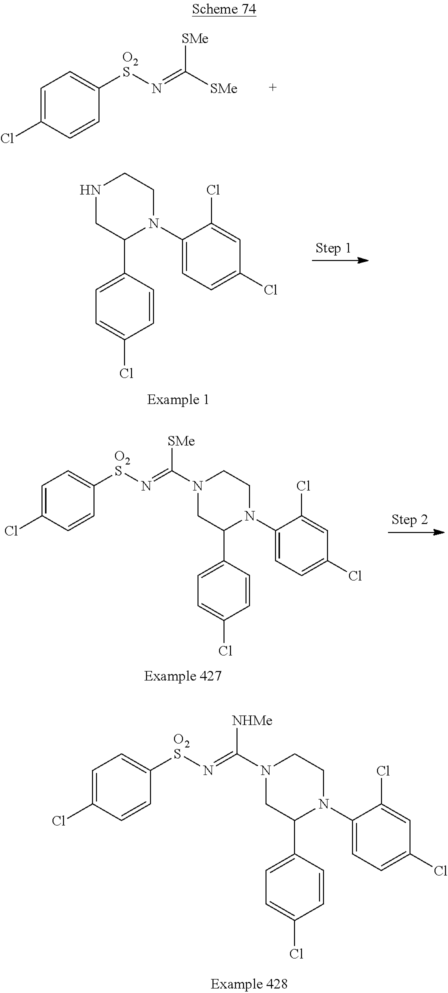 [SMILES: CSC(=NS(=O)(=O)c1ccc(Cl)cc1)SC ; Clc1ccc(C2CNCCN2c2ccc(Cl)cc2Cl)cc1 ; CS/C(=N\S(=O)(=O)c1ccc(Cl)cc1)N1CCN(c2ccc(Cl)cc2Cl)C(c2ccc(Cl)cc2)C1 ; CN/C(=N\S(=O)(=O)c1ccc(Cl)cc1)N1CCN(c2ccc(Cl)cc2Cl)C(c2ccc(Cl)cc2)C1]

### Step 1:

The arylsulfonyldithioimide carbonic acid methyl ester was prepared according to the method of Lange et. al. J. Med Chem. 2004, 47, 627-643 (herein incorporated by reference). To the dithioimide (0.39 g, 1.3 mmol) in propionitrile (4 mL) was added the Et₃N (0.43 mL, 3.1 mmol) and the piperazine Example 1 (0.3 g, 1.3 mmol). The reaction mixture was warmed to reflux and stirred for 20 h, then cooled to room temperature and concentrated in vacuo. The residue, Example 427, was used in step 2 without further purification.

### Step 2:

Example 427 was taken up into MeOH (7 mL) and MeNH₂ (40% solution in water, 1.5 mL, 18 mmol) was added. The reaction mixture was stirred at room temperature for 20 h, then concentrated in vacuo. The resulting residue was taken up into CH₂Cl₂ and the insoluble precipitate was filtered off. The filtrate was concentrated in vacuo and purified by silica gel chromatography (55% EtOAc/hexane) to provide Example 428 (0.35 g, 0.61 mmol).

### Preparation of Examples 429-494

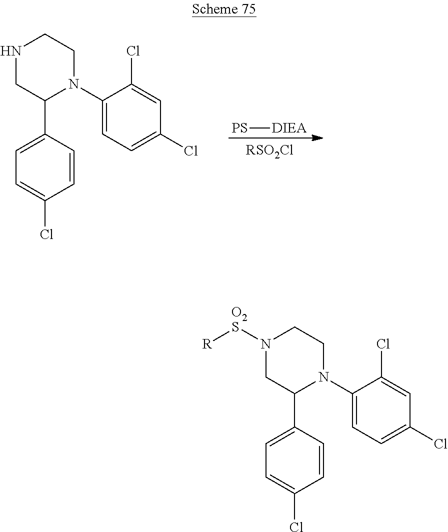

Polystyrene DIEA resin (47 mg, 0.045 mmol) was added to 72-wells of a deep well polypropylene microtiter plate followed by a MeCN/THF (3:2) stock solution (1 mL) of piperazine Example 1 (0.033 mmol). Then 0.5 M stock solutions of each of the individual sulfonyl chlorides (R₁₋₆₆SO₂Cl) (0.135 mL, 0.067 mmol) were added to the wells, which was then sealed and shaken at 25° C. for 20 h. The solutions were filtered thru a polypropylene frit into a second microtiter plate containing polystyrene isocyanate resin (3 equivalents, 0.135 mmol) and polystyrene trisamine resin (6 equivalents, 0.27 mmol). After the top plate was washed with MeCN (0.5 mL), the plate was removed, the bottom microtiter plate sealed and shaken at 25° C. for 16 h. Then the solutions were filtered thru a polypropylene frit into a 96-well collection plate. The wells of the top plate were then washed with MeCN (0.5 mL), and the plate removed. Then the resultant solutions in the collection plate were transferred into vials and the solvents removed in vacuo via a SpeedVac to provide the sulfonamides shown below in Table XV.

TABLE XV

| Example # | Sulfonyl Chloride | Example Structure |
|---|---|---|
| 429 | 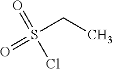 | 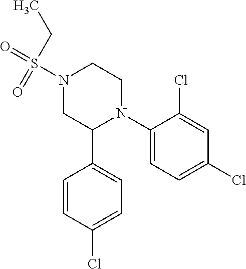 [SMILES: CCS(=O)(=O)N1CCN(c2ccc(Cl)cc2Cl)C(c2ccc(Cl)cc2)C1] |
| 430 | 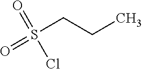 | 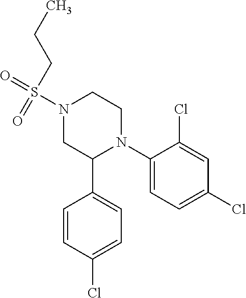 [SMILES: CCCS(=O)(=O)N1CCN(c2ccc(Cl)cc2Cl)C(c2ccc(Cl)cc2)C1] |
| 431 | 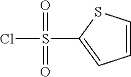 | 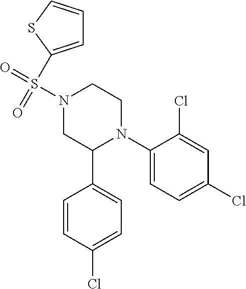 [SMILES: O=S(=O)(c1cccs1)N1CCN(c2ccc(Cl)cc2Cl)C(c2ccc(Cl)cc2)C1] |
| 432 | 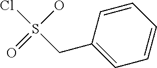 | 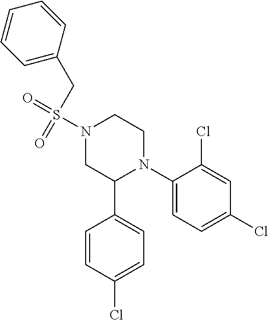 [SMILES: O=S(=O)(Cc1ccccc1)N1CCN(c2ccc(Cl)cc2Cl)C(c2ccc(Cl)cc2)C1] |
| 433 | 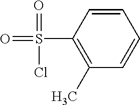 | 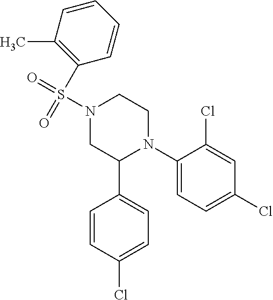 [SMILES: Cc1ccccc1S(=O)(=O)N1CCN(c2ccc(Cl)cc2Cl)C(c2ccc(Cl)cc2)C1] |
| 434 | 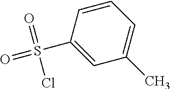 | 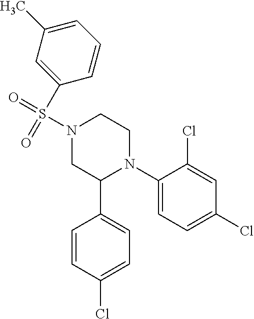 [SMILES: Cc1cccc(S(=O)(=O)N2CCN(c3ccc(Cl)cc3Cl)C(c3ccc(Cl)cc3)C2)c1] |
| 435 | 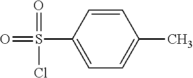 | 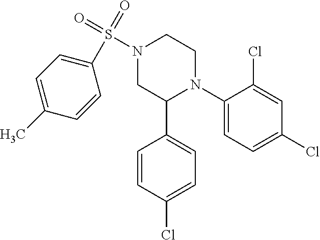 [SMILES: Cc1ccc(S(=O)(=O)N2CCN(c3ccc(Cl)cc3Cl)C(c3ccc(Cl)cc3)C2)cc1] |
| 436 | 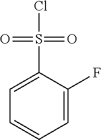 [SMILES: O=S(=O)(Cl)c1ccccc1F] | 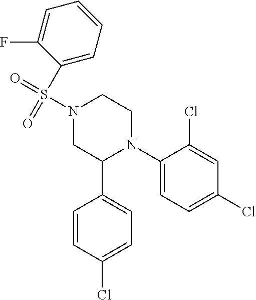 [SMILES: O=S(=O)(c1ccccc1F)N1CCN(c2ccc(Cl)cc2Cl)C(c2ccc(Cl)cc2)C1] |
| 437 | 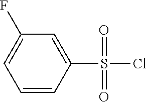 [SMILES: O=S(=O)(Cl)c1cccc(F)c1] | 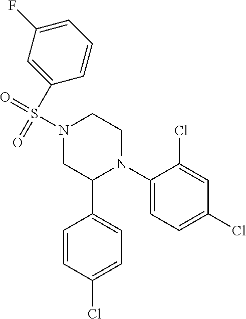 [SMILES: O=S(=O)(c1cccc(F)c1)N1CCN(c2ccc(Cl)cc2Cl)C(c2ccc(Cl)cc2)C1] |
| 438 | 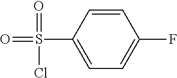 [SMILES: O=S(=O)(Cl)c1ccc(F)cc1] | 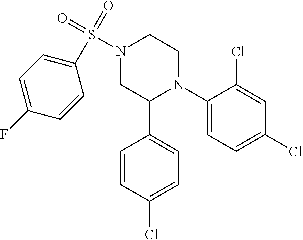 [SMILES: O=S(=O)(c1ccc(F)cc1)N1CCN(c2ccc(Cl)cc2Cl)C(c2ccc(Cl)cc2)C1] |
| 439 | 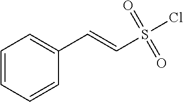 [SMILES: O=S(=O)(Cl)/C=C/c1ccccc1] | 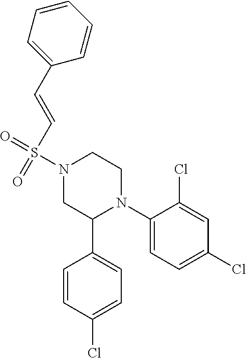 [SMILES: O=S(=O)(/C=C/c1ccccc1)N1CCN(c2ccc(Cl)cc2Cl)C(c2ccc(Cl)cc2)C1] |
| 440 | 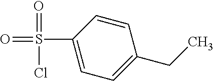 | 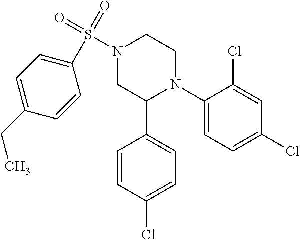 [SMILES: CCc1ccc(S(=O)(=O)N2CCN(c3ccc(Cl)cc3Cl)C(c3ccc(Cl)cc3)C2)cc1] |
| 441 | 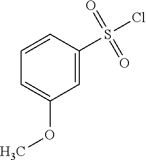 | 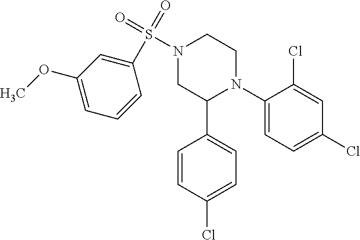 [SMILES: COc1cccc(S(=O)(=O)N2CCN(c3ccc(Cl)cc3Cl)C(c3ccc(Cl)cc3)C2)c1] |
| 442 | 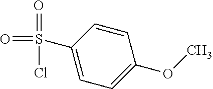 | 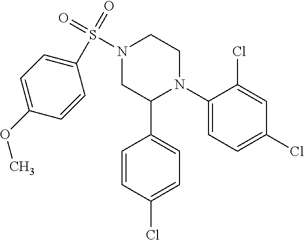 [SMILES: COc1ccc(S(=O)(=O)N2CCN(c3ccc(Cl)cc3Cl)C(c3ccc(Cl)cc3)C2)cc1] |
| 443 | 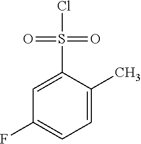 | 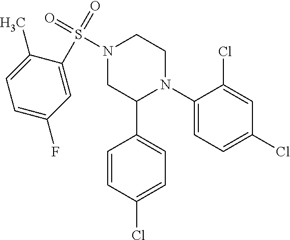 [SMILES: Cc1ccc(F)cc1S(=O)(=O)N1CCN(c2ccc(Cl)cc2Cl)C(c2ccc(Cl)cc2)C1] |
| 444 | 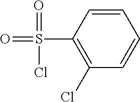 [SMILES: O=S(=O)(Cl)c1ccccc1Cl] | 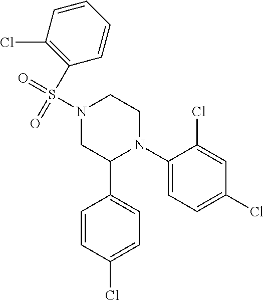 [SMILES: O=S(=O)(c1ccccc1Cl)N1CCN(c2ccc(Cl)cc2Cl)C(c2ccc(Cl)cc2)C1] |
| 445 | 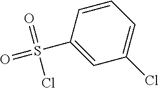 [SMILES: O=S(=O)(Cl)c1cccc(Cl)c1] | 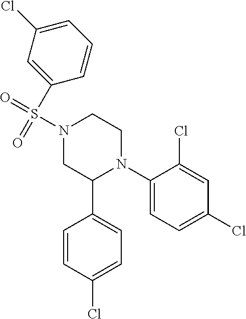 [SMILES: O=S(=O)(c1cccc(Cl)c1)N1CCN(c2ccc(Cl)cc2Cl)C(c2ccc(Cl)cc2)C1] |
| 446 | 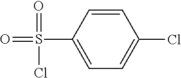 [SMILES: O=S(=O)(Cl)c1ccc(Cl)cc1] | 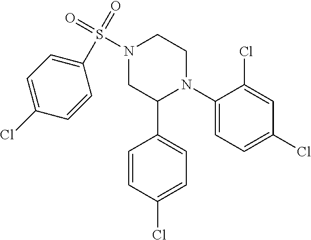 [SMILES: O=S(=O)(c1ccc(Cl)cc1)N1CCN(c2ccc(Cl)cc2Cl)C(c2ccc(Cl)cc2)C1] |
| 447 | 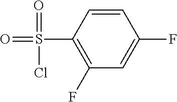 [SMILES: O=S(=O)(Cl)c1ccc(F)cc1F] | 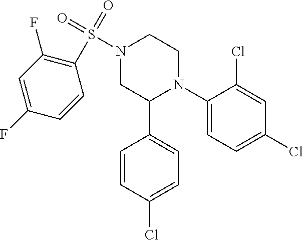 [SMILES: O=S(=O)(c1ccc(F)cc1F)N1CCN(c2ccc(Cl)cc2Cl)C(c2ccc(Cl)cc2)C1] |
| 448 | 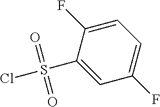 [SMILES: O=S(=O)(Cl)c1cc(F)ccc1F] | 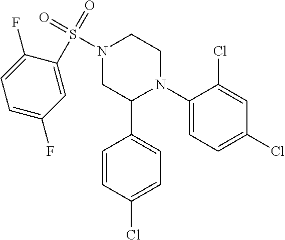 [SMILES: O=S(=O)(c1cc(F)ccc1F)N1CCN(c2ccc(Cl)cc2Cl)C(c2ccc(Cl)cc2)C1] |
| 449 | 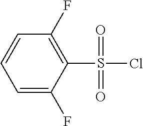 [SMILES: O=S(=O)(Cl)c1c(F)cccc1F] | 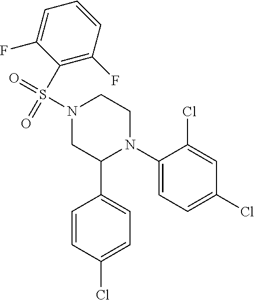 [SMILES: O=S(=O)(c1c(F)cccc1F)N1CCN(c2ccc(Cl)cc2Cl)C(c2ccc(Cl)cc2)C1] |
| 450 | 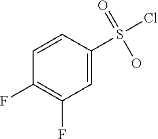 | 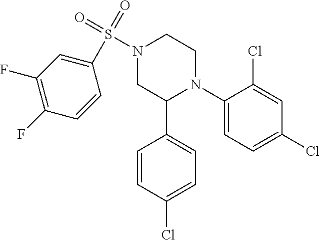 [SMILES: O=S(=O)(c1ccc(F)c(F)c1)N1CCN(c2ccc(Cl)cc2Cl)C(c2ccc(Cl)cc2)C1] |
| 451 | 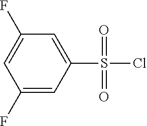 [SMILES: O=S(=O)(Cl)c1cc(F)cc(F)c1] | 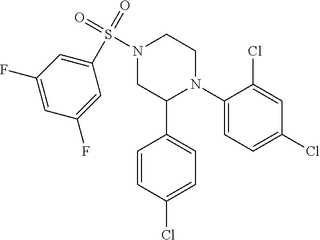 [SMILES: O=S(=O)(c1cc(F)cc(F)c1)N1CCN(c2ccc(Cl)cc2Cl)C(c2ccc(Cl)cc2)C1] |
| 452 | 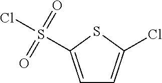 | 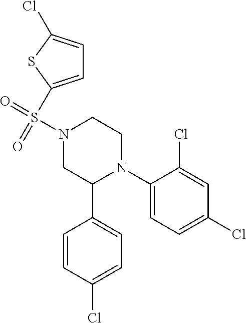 [SMILES: O=S(=O)(c1ccc(Cl)s1)N1CCN(c2ccc(Cl)cc2Cl)C(c2ccc(Cl)cc2)C1] |
| 453 | 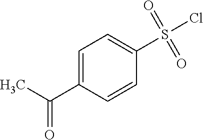 | 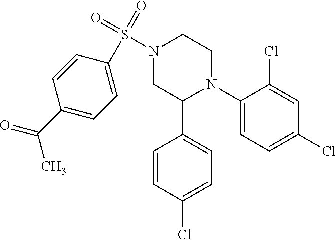 [SMILES: CC(=O)c1ccc(S(=O)(=O)N2CCN(c3ccc(Cl)cc3Cl)C(c3ccc(Cl)cc3)C2)cc1] |
| 454 | 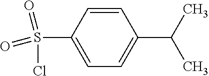 | 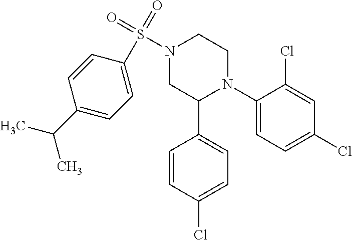 [SMILES: CC(C)c1ccc(S(=O)(=O)N2CCN(c3ccc(Cl)cc3Cl)C(c3ccc(Cl)cc3)C2)cc1] |
| 455 | 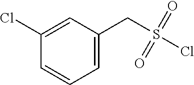 [SMILES: O=S(=O)(Cl)Cc1cccc(Cl)c1] | 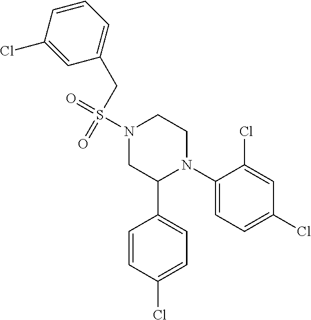 [SMILES: O=S(=O)(Cc1cccc(Cl)c1)N1CCN(c2ccc(Cl)cc2Cl)C(c2ccc(Cl)cc2)C1] |
| 456 | 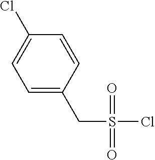 [SMILES: O=S(=O)(Cl)Cc1ccc(Cl)cc1] | 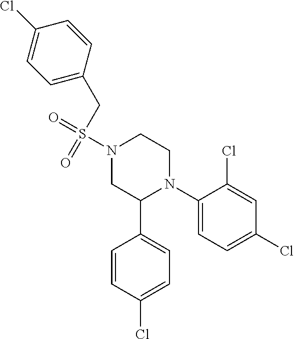 [SMILES: O=S(=O)(Cc1ccc(Cl)cc1)N1CCN(c2ccc(Cl)cc2Cl)C(c2ccc(Cl)cc2)C1] |
| 457 | 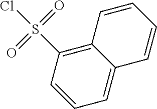 [SMILES: O=S(=O)(Cl)c1cccc2ccccc12] | 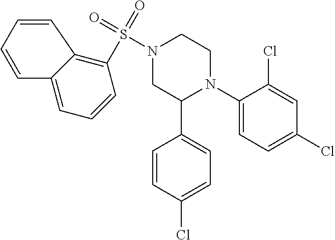 [SMILES: O=S(=O)(c1cccc2ccccc12)N1CCN(c2ccc(Cl)cc2Cl)C(c2ccc(Cl)cc2)C1] |
| 458 | 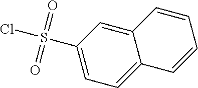 [SMILES: O=S(=O)(Cl)c1ccc2ccccc2c1] | 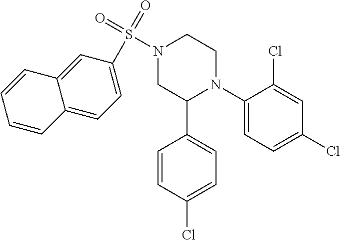 [SMILES: O=S(=O)(c1ccc2ccccc2c1)N1CCN(c2ccc(Cl)cc2Cl)C(c2ccc(Cl)cc2)C1] |
| 459 | 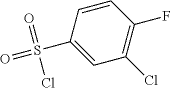 [SMILES: O=S(=O)(Cl)c1ccc(F)c(Cl)c1] | 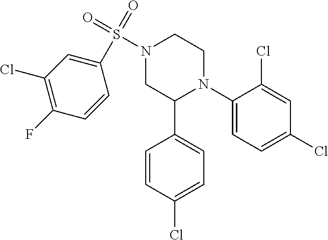 [SMILES: O=S(=O)(c1ccc(F)c(Cl)c1)N1CCN(c2ccc(Cl)cc2Cl)C(c2ccc(Cl)cc2)C1] |
| 460 | 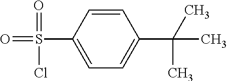 | 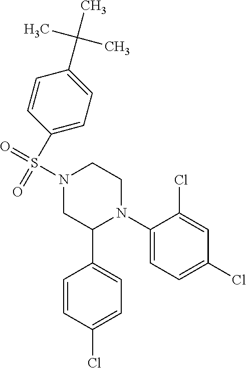 [SMILES: CC(C)(C)c1ccc(S(=O)(=O)N2CCN(c3ccc(Cl)cc3Cl)C(c3ccc(Cl)cc3)C2)cc1] |
| 461 | 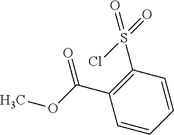 | 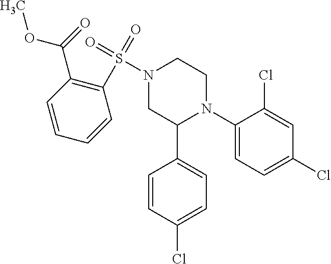 [SMILES: COC(=O)c1ccccc1S(=O)(=O)N1CCN(c2ccc(Cl)cc2Cl)C(c2ccc(Cl)cc2)C1] |
| 462 | 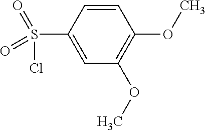 | 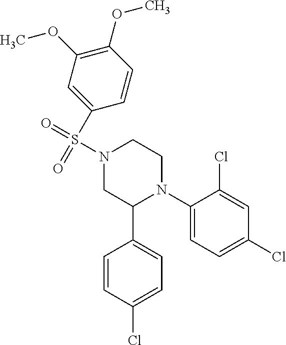 [SMILES: COc1ccc(S(=O)(=O)N2CCN(c3ccc(Cl)cc3Cl)C(c3ccc(Cl)cc3)C2)cc1OC] |
| 463 | 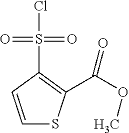 | 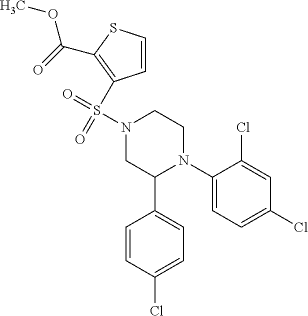 [SMILES: COC(=O)c1sccc1S(=O)(=O)N1CCN(c2ccc(Cl)cc2Cl)C(c2ccc(Cl)cc2)C1] |
| 464 | 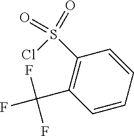 [SMILES: O=S(=O)(Cl)c1ccccc1C(F)(F)F] | 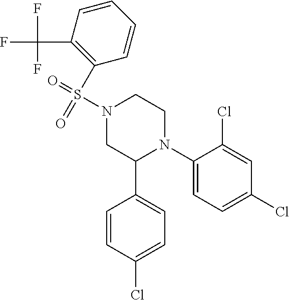 [SMILES: O=S(=O)(c1ccccc1C(F)(F)F)N1CCN(c2ccc(Cl)cc2Cl)C(c2ccc(Cl)cc2)C1] |
| 465 | 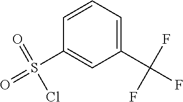 [SMILES: O=S(=O)(Cl)c1cccc(C(F)(F)F)c1] | 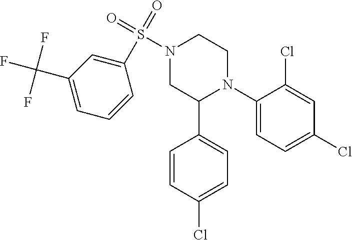 [SMILES: O=S(=O)(c1cccc(C(F)(F)F)c1)N1CCN(c2ccc(Cl)cc2Cl)C(c2ccc(Cl)cc2)C1] |
| 466 | 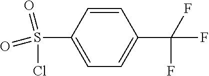 [SMILES: O=S(=O)(Cl)c1ccc(C(F)(F)F)cc1] | 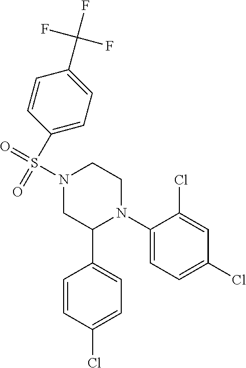 [SMILES: O=S(=O)(c1ccc(C(F)(F)F)cc1)N1CCN(c2ccc(Cl)cc2Cl)C(c2ccc(Cl)cc2)C1] |
| 467 | 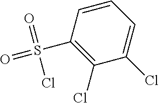 [SMILES: O=S(=O)(Cl)c1cccc(Cl)c1Cl] | 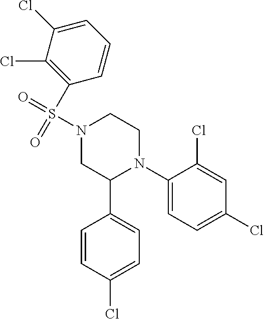 [SMILES: O=S(=O)(c1cccc(Cl)c1Cl)N1CCN(c2ccc(Cl)cc2Cl)C(c2ccc(Cl)cc2)C1] |
| 468 | 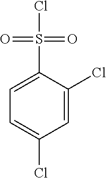 [SMILES: O=S(=O)(Cl)c1ccc(Cl)cc1Cl] | 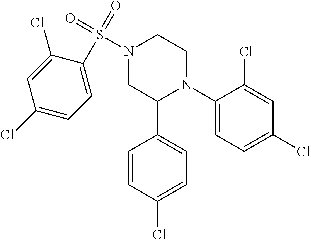 [SMILES: O=S(=O)(c1ccc(Cl)cc1Cl)N1CCN(c2ccc(Cl)cc2Cl)C(c2ccc(Cl)cc2)C1] |
| 469 | 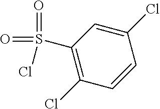 [SMILES: O=S(=O)(Cl)c1cc(Cl)ccc1Cl] | 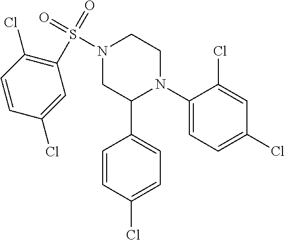 [SMILES: O=S(=O)(c1cc(Cl)ccc1Cl)N1CCN(c2ccc(Cl)cc2Cl)C(c2ccc(Cl)cc2)C1] |
| 470 | 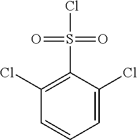 [SMILES: O=S(=O)(Cl)c1c(Cl)cccc1Cl] | 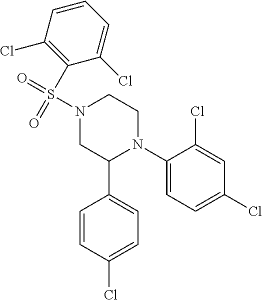 [SMILES: O=S(=O)(c1c(Cl)cccc1Cl)N1CCN(c2ccc(Cl)cc2Cl)C(c2ccc(Cl)cc2)C1] |
| 471 | 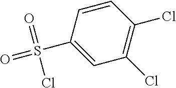 [SMILES: O=S(=O)(Cl)c1ccc(Cl)c(Cl)c1] | 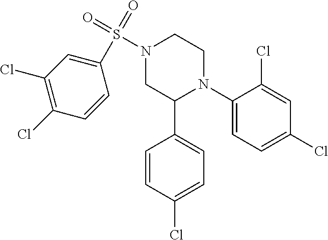 [SMILES: O=S(=O)(c1ccc(Cl)c(Cl)c1)N1CCN(c2ccc(Cl)cc2Cl)C(c2ccc(Cl)cc2)C1] |
| 472 | 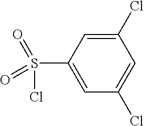 [SMILES: O=S(=O)(Cl)c1cc(Cl)cc(Cl)c1] | 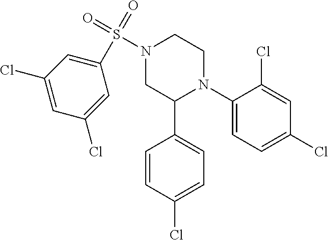 [SMILES: O=S(=O)(c1cc(Cl)cc(Cl)c1)N1CCN(c2ccc(Cl)cc2Cl)C(c2ccc(Cl)cc2)C1] |
| 473 | 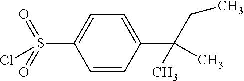 | 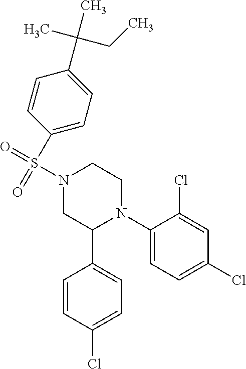 [SMILES: CCC(C)(C)c1ccc(S(=O)(=O)N2CCN(c3ccc(Cl)cc3Cl)C(c3ccc(Cl)cc3)C2)cc1] |
| 474 | 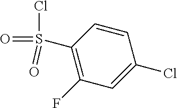 [SMILES: O=S(=O)(Cl)c1ccc(Cl)cc1F] | 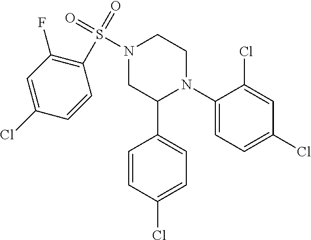 [SMILES: O=S(=O)(c1ccc(Cl)cc1F)N1CCN(c2ccc(Cl)cc2Cl)C(c2ccc(Cl)cc2)C1] |
| 475 | 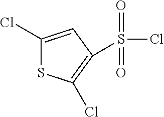 [SMILES: O=S(=O)(Cl)c1cc(Cl)sc1Cl] | 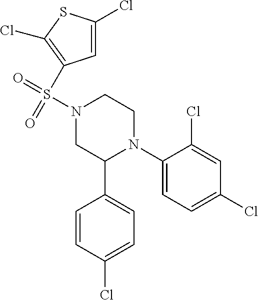 [SMILES: O=S(=O)(c1cc(Cl)sc1Cl)N1CCN(c2ccc(Cl)cc2Cl)C(c2ccc(Cl)cc2)C1] |
| 476 | 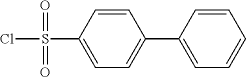 [SMILES: O=S(=O)(Cl)c1ccc(-c2ccccc2)cc1] | 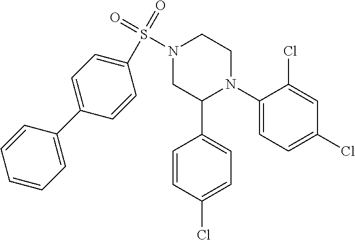 [SMILES: O=S(=O)(c1ccc(-c2ccccc2)cc1)N1CCN(c2ccc(Cl)cc2Cl)C(c2ccc(Cl)cc2)C1] |
| 477 | 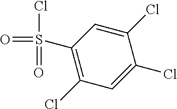 [SMILES: O=S(=O)(Cl)c1cc(Cl)c(Cl)cc1Cl] | 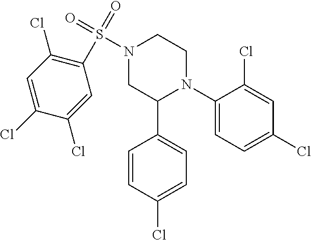 [SMILES: O=S(=O)(c1cc(Cl)c(Cl)cc1Cl)N1CCN(c2ccc(Cl)cc2Cl)C(c2ccc(Cl)cc2)C1] |
| 478 | 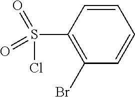 [SMILES: O=S(=O)(Cl)c1ccccc1Br] | 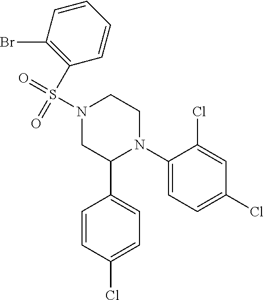 [SMILES: O=S(=O)(c1ccccc1Br)N1CCN(c2ccc(Cl)cc2Cl)C(c2ccc(Cl)cc2)C1] |
| 479 | 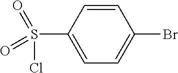 [SMILES: O=S(=O)(Cl)c1ccc(Br)cc1] | 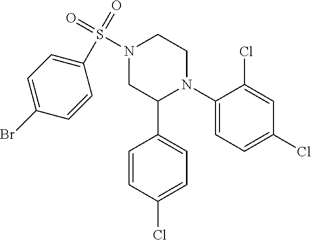 [SMILES: O=S(=O)(c1ccc(Br)cc1)N1CCN(c2ccc(Cl)cc2Cl)C(c2ccc(Cl)cc2)C1] |
| 480 | 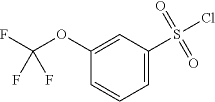 [SMILES: O=S(=O)(Cl)c1cccc(OC(F)(F)F)c1] | 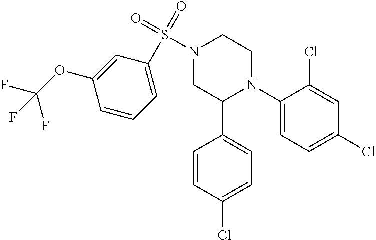 [SMILES: O=S(=O)(c1cccc(OC(F)(F)F)c1)N1CCN(c2ccc(Cl)cc2Cl)C(c2ccc(Cl)cc2)C1] |
| 481 | 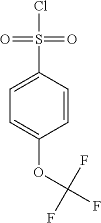 [SMILES: O=S(=O)(Cl)c1ccc(OC(F)(F)F)cc1] | 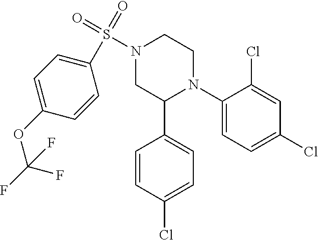 [SMILES: O=S(=O)(c1ccc(OC(F)(F)F)cc1)N1CCN(c2ccc(Cl)cc2Cl)C(c2ccc(Cl)cc2)C1] |
| 482 | 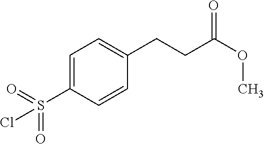 [SMILES: COC(=O)CCc1ccc(S(=O)(=O)Cl)cc1] | 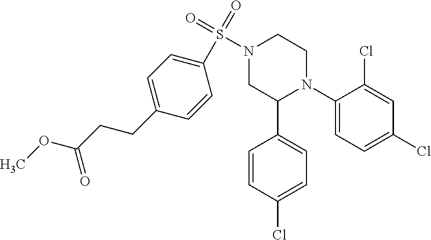 [SMILES: COC(=O)CCc1ccc(S(=O)(=O)N2CCN(c3ccc(Cl)cc3Cl)C(c3ccc(Cl)cc3)C2)cc1] |
| 483 | 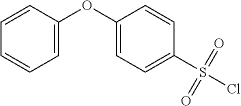 [SMILES: O=S(=O)(Cl)c1ccc(Oc2ccccc2)cc1] | 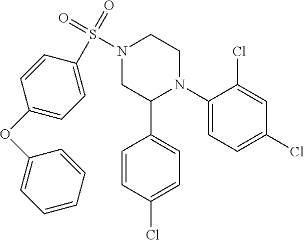 [SMILES: O=S(=O)(c1ccc(Oc2ccccc2)cc1)N1CCN(c2ccc(Cl)cc2Cl)C(c2ccc(Cl)cc2)C1] |
| 484 | 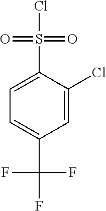 [SMILES: O=S(=O)(Cl)c1ccc(C(F)(F)F)cc1Cl] | 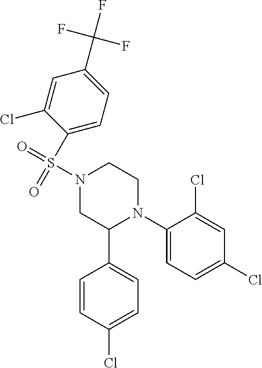 [SMILES: O=S(=O)(c1ccc(C(F)(F)F)cc1Cl)N1CCN(c2ccc(Cl)cc2Cl)C(c2ccc(Cl)cc2)C1] |
| 485 | 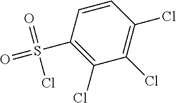 [SMILES: O=S(=O)(Cl)c1ccc(Cl)c(Cl)c1Cl] | 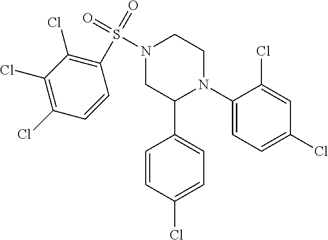 [SMILES: O=S(=O)(c1ccc(Cl)c(Cl)c1Cl)N1CCN(c2ccc(Cl)cc2Cl)C(c2ccc(Cl)cc2)C1] |
| 486 | 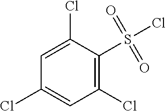 [SMILES: O=S(=O)(Cl)c1c(Cl)cc(Cl)cc1Cl] | 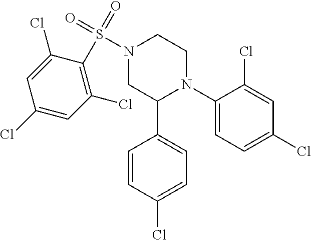 [SMILES: O=S(=O)(c1c(Cl)cc(Cl)cc1Cl)N1CCN(c2ccc(Cl)cc2Cl)C(c2ccc(Cl)cc2)C1] |
| 487 | 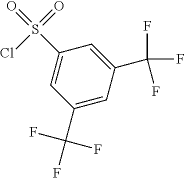 [SMILES: O=S(=O)(Cl)c1cc(C(F)(F)F)cc(C(F)(F)F)c1] | 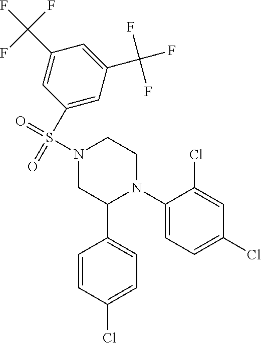 [SMILES: O=S(=O)(c1cc(C(F)(F)F)cc(C(F)(F)F)c1)N1CCN(c2ccc(Cl)cc2Cl)C(c2ccc(Cl)cc2)C1] |
| 488 | 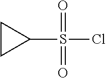 | 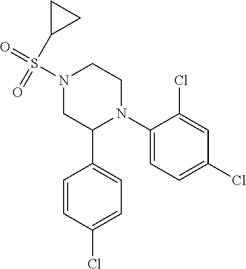 [SMILES: O=S(=O)(C1CC1)N1CCN(c2ccc(Cl)cc2Cl)C(c2ccc(Cl)cc2)C1] |
| 489 | 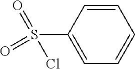 | 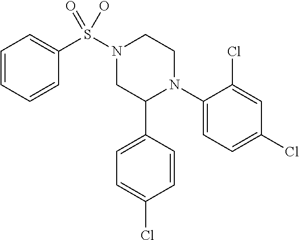 |
| 490 | 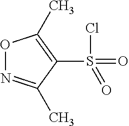 | 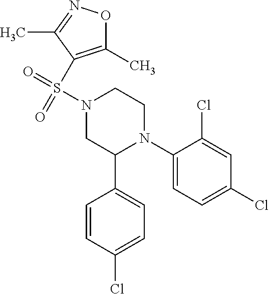 [SMILES: Cc1noc(C)c1S(=O)(=O)N1CCN(c2ccc(Cl)cc2Cl)C(c2ccc(Cl)cc2)C1] |
| 491 | 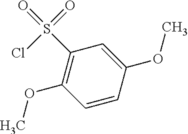 | 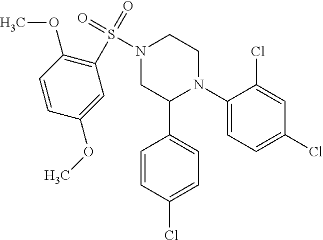 [SMILES: COc1ccc(OC)c(S(=O)(=O)N2CCN(c3ccc(Cl)cc3Cl)C(c3ccc(Cl)cc3)C2)c1] |
| 492 | 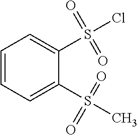 | 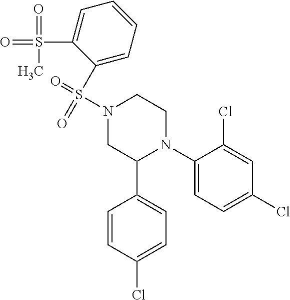 [SMILES: CS(=O)(=O)c1ccccc1S(=O)(=O)N1CCN(c2ccc(Cl)cc2Cl)C(c2ccc(Cl)cc2)C1] |
| 493 | 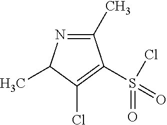 | 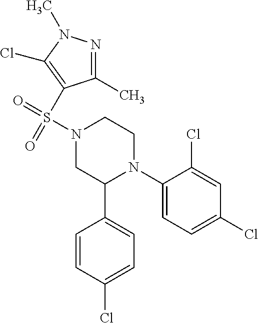 [SMILES: Cc1nn(C)c(Cl)c1S(=O)(=O)N1CCN(c2ccc(Cl)cc2Cl)C(c2ccc(Cl)cc2)C1] |
| 494 | 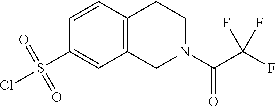 [SMILES: O=C(N1CCc2ccc(S(=O)(=O)Cl)cc2C1)C(F)(F)F] | 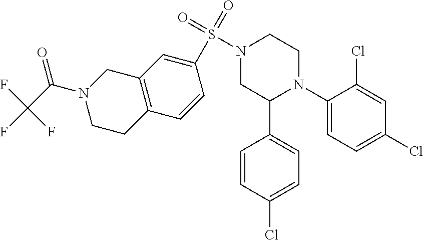 [SMILES: O=C(N1CCc2ccc(S(=O)(=O)N3CCN(c4ccc(Cl)cc4Cl)C(c4ccc(Cl)cc4)C3)cc2C1)C(F)(F)F] |

### Preparation of Examples 495-497

Examples 495-497 were prepared by the reaction of the appropriate piperazine and aldehyde (e.g., by the method of step 4 of Scheme 64), shown in Table XVI, below. The piperazines were prepared, e.g., by the methods described in Scheme 71.

TABLE XVI

| Example # | Piperazine | Aldehyde | Example Structure |
|---|---|---|---|
| 495 | 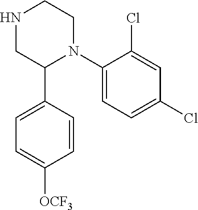 [SMILES: FC(F)(F)Oc1ccc(C2CNCCN2c2ccc(Cl)cc2Cl)cc1] | 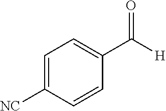 | 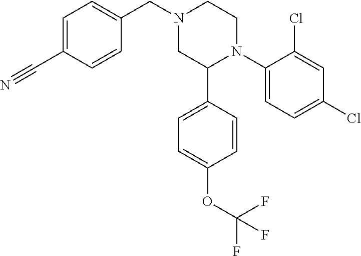 [SMILES: N#Cc1ccc(CN2CCN(c3ccc(Cl)cc3Cl)C(c3ccc(OC(F)(F)F)cc3)C2)cc1] |
| 496 | 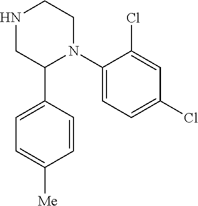 [SMILES: Cc1ccc(C2CNCCN2c2ccc(Cl)cc2Cl)cc1] | 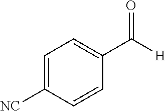 | 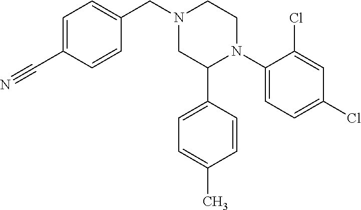 [SMILES: Cc1ccc(C2CN(Cc3ccc(C#N)cc3)CCN2c2ccc(Cl)cc2Cl)cc1] |
| 497 | 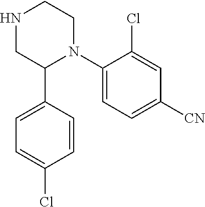 [SMILES: N#Cc1ccc(N2CCNCC2c2ccc(Cl)cc2)c(Cl)c1] | 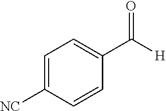 | 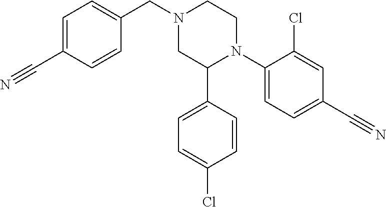 [SMILES: N#Cc1ccc(CN2CCN(c3ccc(C#N)cc3Cl)C(c3ccc(Cl)cc3)C2)cc1] |

### Preparation of Examples 498-513

Examples 498-513 were prepared by the reaction of the appropriate piperazine and aldehyde (e.g., by the method of Scheme 64, step 4) shown in Table XVII, below. The piperazines were prepared, e.g., by the method of Scheme 28.

TABLE XVII

| Ex- | | | |
|---|---|---|---|
| ample # | Piperazine | Aldehyde | Example Structure |
| 498 | 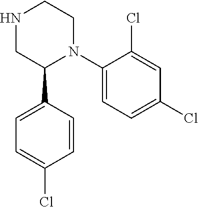 [SMILES: Clc1ccc([C@H]2CNCCN2c2ccc(Cl)cc2Cl)cc1] | 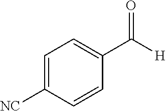 | 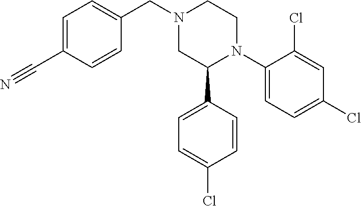 [SMILES: N#Cc1ccc(CN2CCN(c3ccc(Cl)cc3Cl)[C@@H](c3ccc(Cl)cc3)C2)cc1] |
| 499 | 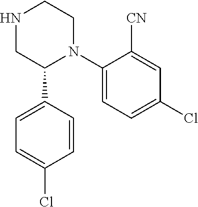 [SMILES: N#Cc1cc(Cl)ccc1N1CCNC[C@H]1c1ccc(Cl)cc1] | 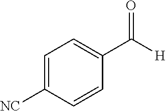 | 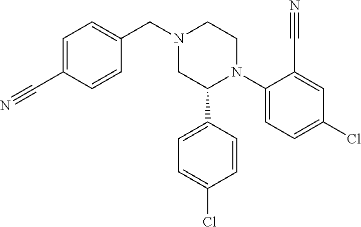 [SMILES: N#Cc1ccc(CN2CCN(c3ccc(Cl)cc3C#N)[C@H](c3ccc(Cl)cc3)C2)cc1] |
| 500 | 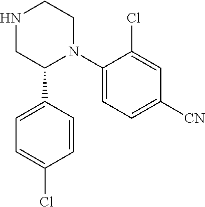 [SMILES: N#Cc1ccc(N2CCNC[C@H]2c2ccc(Cl)cc2)c(Cl)c1] | 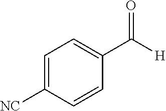 | 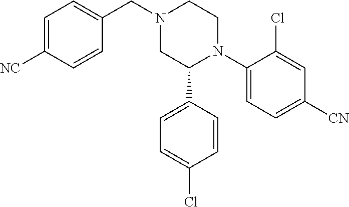 [SMILES: N#Cc1ccc(CN2CCN(c3ccc(C#N)cc3Cl)[C@H](c3ccc(Cl)cc3)C2)cc1] |
| 501 | 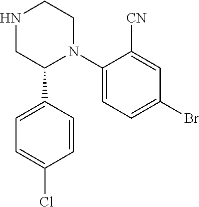 [SMILES: N#Cc1cc(Br)ccc1N1CCNC[C@H]1c1ccc(Cl)cc1] | 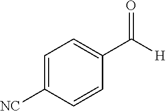 | 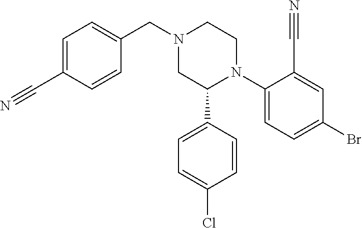 [SMILES: N#Cc1ccc(CN2CCN(c3ccc(Br)cc3C#N)[C@H](c3ccc(Cl)cc3)C2)cc1] |
| 502 | 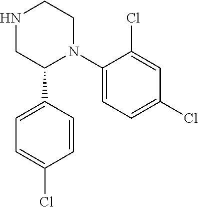 [SMILES: Clc1ccc([C@@H]2CNCCN2c2ccc(Cl)cc2Cl)cc1] | 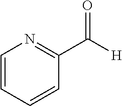 | 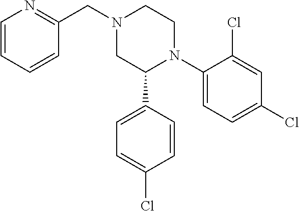 [SMILES: Clc1ccc([C@@H]2CN(Cc3ccccn3)CCN2c2ccc(Cl)cc2Cl)cc1] |
| 503 | 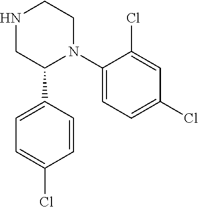 [SMILES: Clc1ccc([C@@H]2CNCCN2c2ccc(Cl)cc2Cl)cc1] | 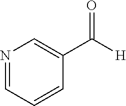 | 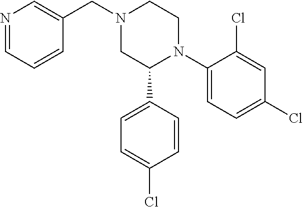 [SMILES: Clc1ccc([C@@H]2CN(Cc3cccnc3)CCN2c2ccc(Cl)cc2Cl)cc1] |
| 504 | 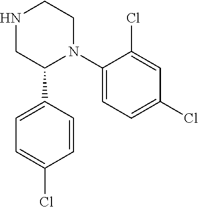 [SMILES: Clc1ccc([C@@H]2CNCCN2c2ccc(Cl)cc2Cl)cc1] | 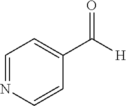 | 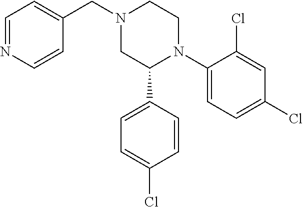 [SMILES: Clc1ccc([C@@H]2CN(Cc3ccncc3)CCN2c2ccc(Cl)cc2Cl)cc1] |
| 505 | 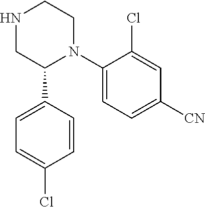 [SMILES: N#Cc1ccc(N2CCNC[C@H]2c2ccc(Cl)cc2)c(Cl)c1] | 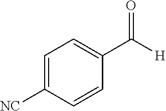 | 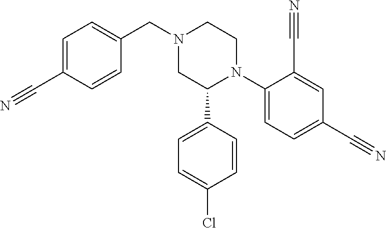 [SMILES: N#Cc1ccc(CN2CCN(c3ccc(C#N)cc3C#N)[C@H](c3ccc(Cl)cc3)C2)cc1] |
| 506 | 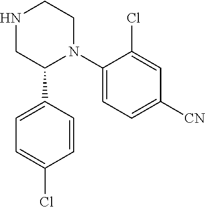 [SMILES: N#Cc1ccc(N2CCNC[C@H]2c2ccc(Cl)cc2)c(Cl)c1] | 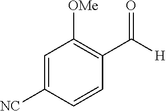 [SMILES: COc1cc(C#N)ccc1C=O] | 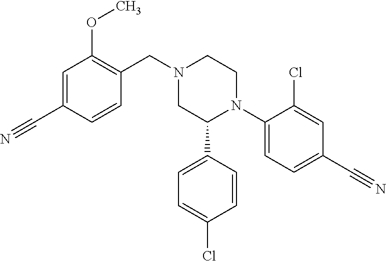 [SMILES: COc1cc(C#N)ccc1CN1CCN(c2ccc(C#N)cc2Cl)[C@H](c2ccc(Cl)cc2)C1] |
| 507 | 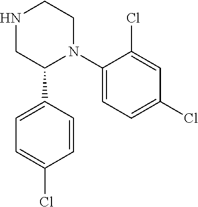 [SMILES: Clc1ccc([C@@H]2CNCCN2c2ccc(Cl)cc2Cl)cc1] | 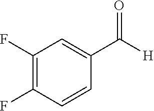 | 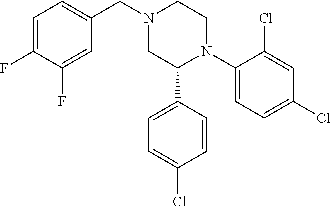 [SMILES: Fc1ccc(CN2CCN(c3ccc(Cl)cc3Cl)[C@H](c3ccc(Cl)cc3)C2)cc1F] |
| 508 | 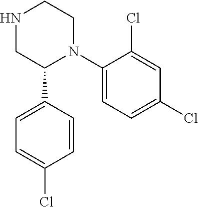 [SMILES: Clc1ccc([C@@H]2CNCCN2c2ccc(Cl)cc2Cl)cc1] | 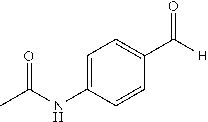 | 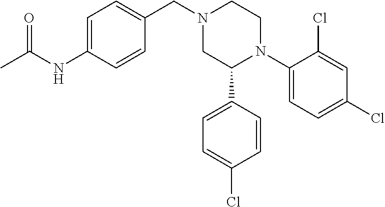 [SMILES: CC(=O)Nc1ccc(CN2CCN(c3ccc(Cl)cc3Cl)[C@H](c3ccc(Cl)cc3)C2)cc1] |
| 509 | 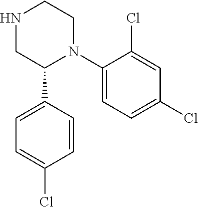 [SMILES: Clc1ccc([C@@H]2CNCCN2c2ccc(Cl)cc2Cl)cc1] | 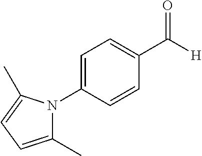 | 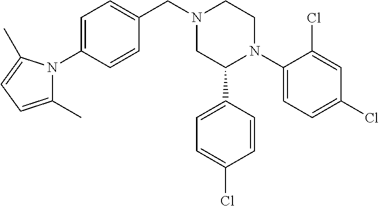 [SMILES: Cc1ccc(C)n1-c1ccc(CN2CCN(c3ccc(Cl)cc3Cl)[C@H](c3ccc(Cl)cc3)C2)cc1] |
| 510 | 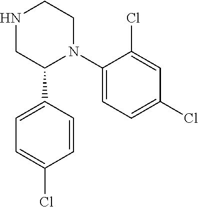 [SMILES: Clc1ccc([C@@H]2CNCCN2c2ccc(Cl)cc2Cl)cc1] | 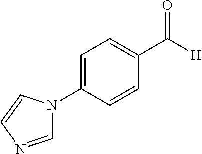 [SMILES: O=Cc1ccc(-n2ccnc2)cc1] | 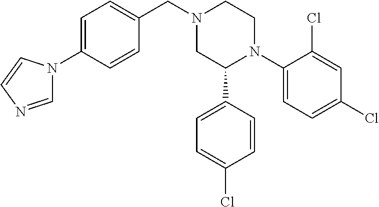 [SMILES: Clc1ccc([C@@H]2CN(Cc3ccc(-n4ccnc4)cc3)CCN2c2ccc(Cl)cc2Cl)cc1] |
| 511 | 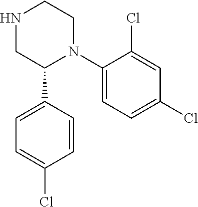 [SMILES: Clc1ccc([C@@H]2CNCCN2c2ccc(Cl)cc2Cl)cc1] | 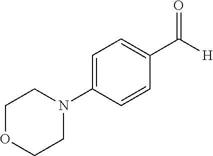 [SMILES: O=Cc1ccc(N2CCOCC2)cc1] | 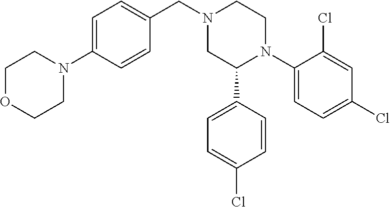 [SMILES: Clc1ccc([C@@H]2CN(Cc3ccc(N4CCOCC4)cc3)CCN2c2ccc(Cl)cc2Cl)cc1] |
| 512 | 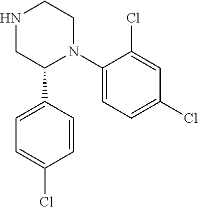 [SMILES: Clc1ccc([C@@H]2CNCCN2c2ccc(Cl)cc2Cl)cc1] | 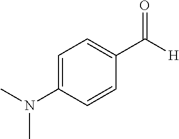 | 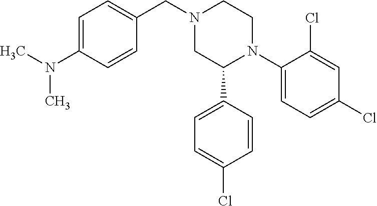 [SMILES: CN(C)c1ccc(CN2CCN(c3ccc(Cl)cc3Cl)[C@H](c3ccc(Cl)cc3)C2)cc1] |
| 513 | 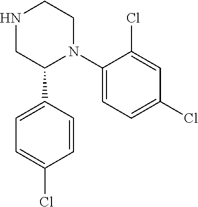 [SMILES: Clc1ccc([C@@H]2CNCCN2c2ccc(Cl)cc2Cl)cc1] | 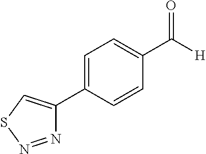 [SMILES: O=Cc1ccc(-c2csnn2)cc1] | 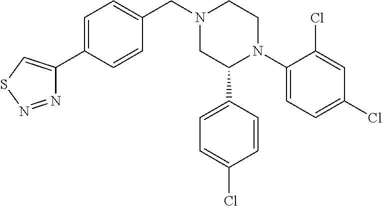 [SMILES: Clc1ccc([C@@H]2CN(Cc3ccc(-c4csnn4)cc3)CCN2c2ccc(Cl)cc2Cl)cc1] |

### Preparation of Examples 514-530

Examples 514-530 were prepared by the reaction of the appropriate piperazine and carboxylic acid (e.g., the method of Scheme 17) shown in Table XVIII, below.

TABLE XVIII

| Example # | Piperazine | Carboxylic Acid | Example Structure |
|---|---|---|---|
| 514 | 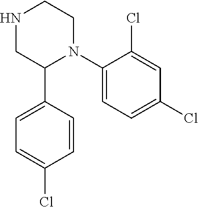 [SMILES: Clc1ccc(C2CNCCN2c2ccc(Cl)cc2Cl)cc1] | 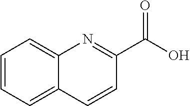 [SMILES: O=C(O)c1ccc2ccccc2n1] | 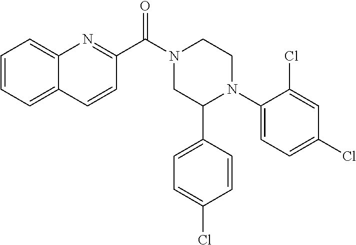 [SMILES: O=C(c1ccc2ccccc2n1)N1CCN(c2ccc(Cl)cc2Cl)C(c2ccc(Cl)cc2)C1] |
| 515 | 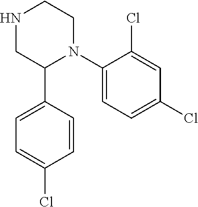 [SMILES: Clc1ccc(C2CNCCN2c2ccc(Cl)cc2Cl)cc1] | 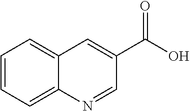 [SMILES: O=C(O)c1cnc2ccccc2c1] | 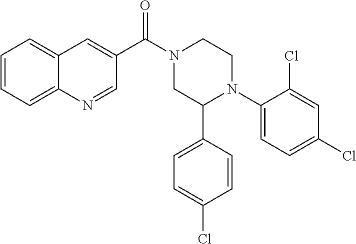 [SMILES: O=C(c1cnc2ccccc2c1)N1CCN(c2ccc(Cl)cc2Cl)C(c2ccc(Cl)cc2)C1] |
| 516 | 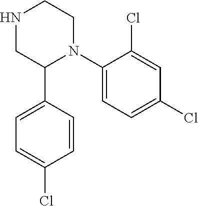 [SMILES: Clc1ccc(C2CNCCN2c2ccc(Cl)cc2Cl)cc1] | 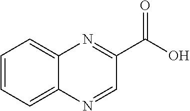 [SMILES: O=C(O)c1cnc2ccccc2n1] | 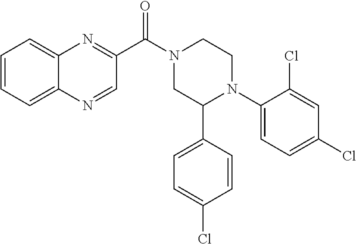 [SMILES: O=C(c1cnc2ccccc2n1)N1CCN(c2ccc(Cl)cc2Cl)C(c2ccc(Cl)cc2)C1] |
| 517 | 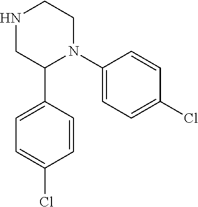 [SMILES: Clc1ccc(C2CNCCN2c2ccc(Cl)cc2)cc1] | 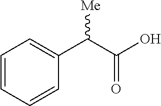 [SMILES: CC(C(=O)O)c1ccccc1] | 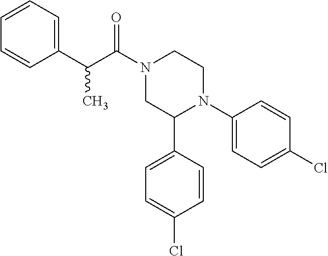 [SMILES: CC(C(=O)N1CCN(c2ccc(Cl)cc2)C(c2ccc(Cl)cc2)C1)c1ccccc1] |
| 518 | 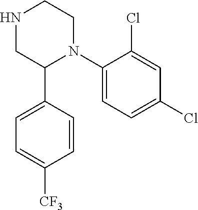 [SMILES: FC(F)(F)c1ccc(C2CNCCN2c2ccc(Cl)cc2Cl)cc1] | 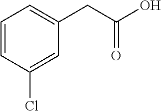 [SMILES: O=C(O)Cc1cccc(Cl)c1] | 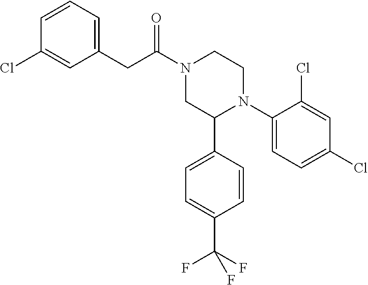 [SMILES: O=C(Cc1cccc(Cl)c1)N1CCN(c2ccc(Cl)cc2Cl)C(c2ccc(C(F)(F)F)cc2)C1] |
| 519 | 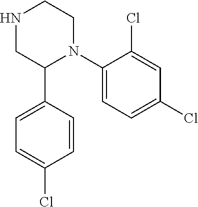 [SMILES: Clc1ccc(C2CNCCN2c2ccc(Cl)cc2Cl)cc1] | 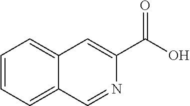 [SMILES: O=C(O)c1cc2ccccc2cn1] | 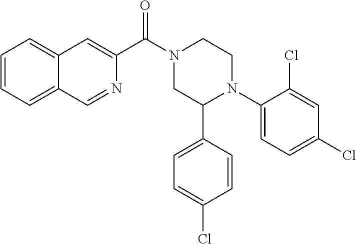 [SMILES: O=C(c1cc2ccccc2cn1)N1CCN(c2ccc(Cl)cc2Cl)C(c2ccc(Cl)cc2)C1] |
| 520 | 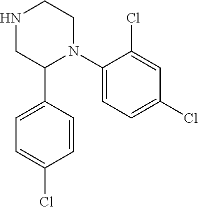 [SMILES: Clc1ccc(C2CNCCN2c2ccc(Cl)cc2Cl)cc1] | 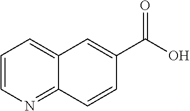 [SMILES: O=C(O)c1ccc2ncccc2c1] | 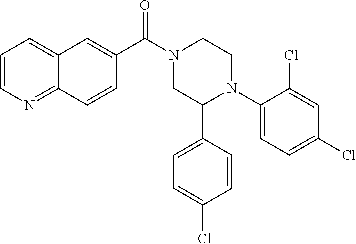 [SMILES: O=C(c1ccc2ncccc2c1)N1CCN(c2ccc(Cl)cc2Cl)C(c2ccc(Cl)cc2)C1] |
| 521 | 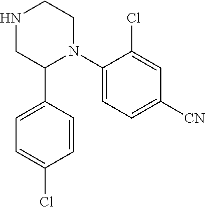 [SMILES: N#Cc1ccc(N2CCNCC2c2ccc(Cl)cc2)c(Cl)c1] | 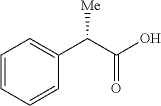 [SMILES: C[C@H](C(=O)O)c1ccccc1] | 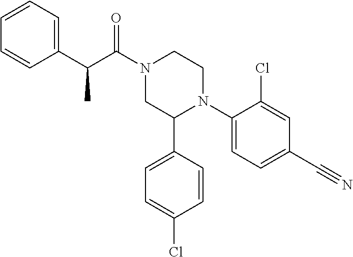 [SMILES: C[C@H](C(=O)N1CCN(c2ccc(C#N)cc2Cl)C(c2ccc(Cl)cc2)C1)c1ccccc1] |
| 522 | 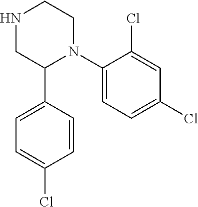 [SMILES: Clc1ccc(C2CNCCN2c2ccc(Cl)cc2Cl)cc1] | 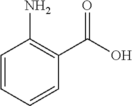 | 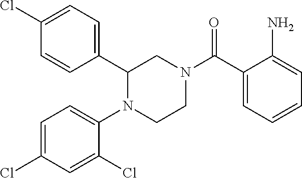 [SMILES: Nc1ccccc1C(=O)N1CCN(c2ccc(Cl)cc2Cl)C(c2ccc(Cl)cc2)C1] |
| 523 | 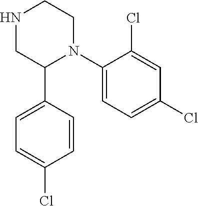 [SMILES: Clc1ccc(C2CNCCN2c2ccc(Cl)cc2Cl)cc1] | 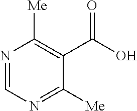 [SMILES: Cc1ncnc(C)c1C(=O)O] | 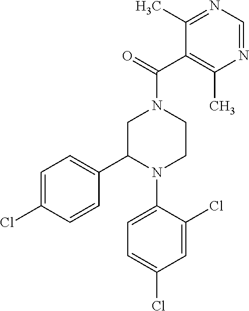 [SMILES: Cc1ncnc(C)c1C(=O)N1CCN(c2ccc(Cl)cc2Cl)C(c2ccc(Cl)cc2)C1] |
| 524 | 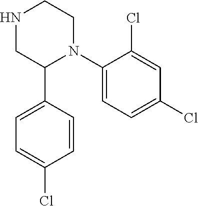 [SMILES: Clc1ccc(C2CNCCN2c2ccc(Cl)cc2Cl)cc1] | 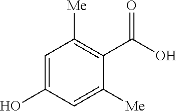 [SMILES: Cc1cc(O)cc(C)c1C(=O)O] | 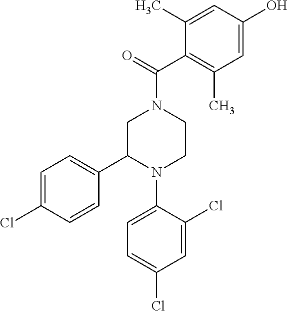 [SMILES: Cc1cc(O)cc(C)c1C(=O)N1CCN(c2ccc(Cl)cc2Cl)C(c2ccc(Cl)cc2)C1] |
| 525 | 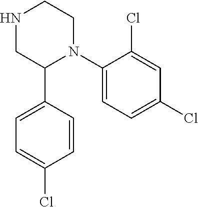 [SMILES: Clc1ccc(C2CNCCN2c2ccc(Cl)cc2Cl)cc1] | 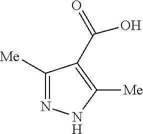 | 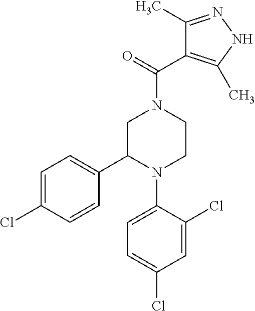 [SMILES: Cc1n[nH]c(C)c1C(=O)N1CCN(c2ccc(Cl)cc2Cl)C(c2ccc(Cl)cc2)C1] |
| 526 | 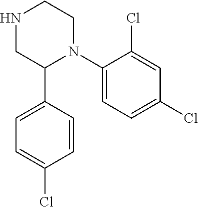 [SMILES: Clc1ccc(C2CNCCN2c2ccc(Cl)cc2Cl)cc1] | 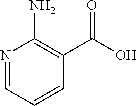 | 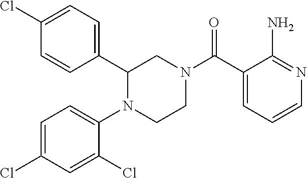 [SMILES: Nc1ncccc1C(=O)N1CCN(c2ccc(Cl)cc2Cl)C(c2ccc(Cl)cc2)C1] |
| 527 | 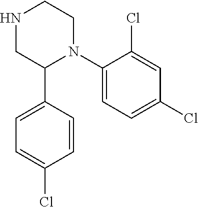 [SMILES: Clc1ccc(C2CNCCN2c2ccc(Cl)cc2Cl)cc1] | 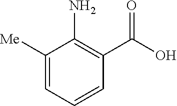 [SMILES: Cc1cccc(C(=O)O)c1N] | 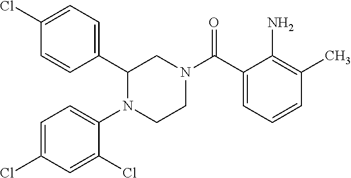 [SMILES: Cc1cccc(C(=O)N2CCN(c3ccc(Cl)cc3Cl)C(c3ccc(Cl)cc3)C2)c1N] |
| 528 | 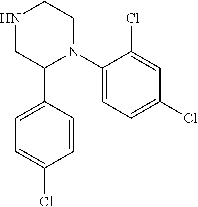 [SMILES: Clc1ccc(C2CNCCN2c2ccc(Cl)cc2Cl)cc1] | 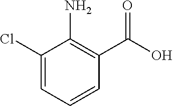 [SMILES: Nc1c(Cl)cccc1C(=O)O] | 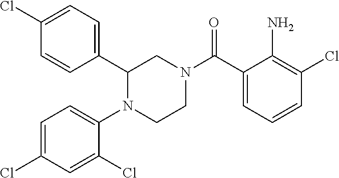 [SMILES: Nc1c(Cl)cccc1C(=O)N1CCN(c2ccc(Cl)cc2Cl)C(c2ccc(Cl)cc2)C1] |
| 529 | 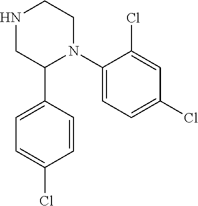 [SMILES: Clc1ccc(C2CNCCN2c2ccc(Cl)cc2Cl)cc1] | 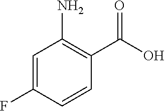 [SMILES: Nc1cc(F)ccc1C(=O)O] | 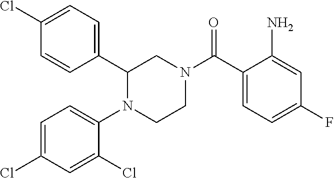 [SMILES: Nc1cc(F)ccc1C(=O)N1CCN(c2ccc(Cl)cc2Cl)C(c2ccc(Cl)cc2)C1] |
| 530 | 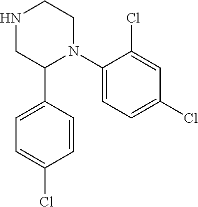 [SMILES: Clc1ccc(C2CNCCN2c2ccc(Cl)cc2Cl)cc1] | 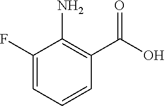 [SMILES: Nc1c(F)cccc1C(=O)O] | 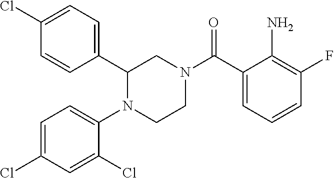 [SMILES: Nc1c(F)cccc1C(=O)N1CCN(c2ccc(Cl)cc2Cl)C(c2ccc(Cl)cc2)C1] |

### Preparation of Examples 531-570

Examples 531-570 were prepared by the reaction of the appropriate piperazine (e.g., Examples 304 or 426) and carboxylic acid shown in Table XIX, below (e.g., by the method of Scheme 17).

TABLE XIX

| Ex- | | | |
|---|---|---|---|
| ample # | Piperazine | Carboxylic Acid | Example Structure |
| 531 | 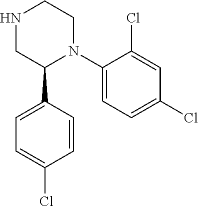 [SMILES: Clc1ccc([C@H]2CNCCN2c2ccc(Cl)cc2Cl)cc1] | 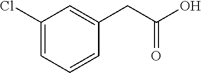 [SMILES: O=C(O)Cc1cccc(Cl)c1] | 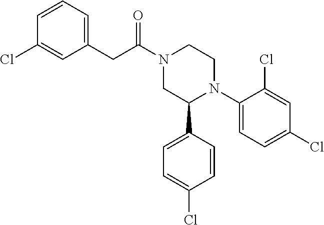 [SMILES: O=C(Cc1cccc(Cl)c1)N1CCN(c2ccc(Cl)cc2Cl)[C@@H](c2ccc(Cl)cc2)C1] |
| 532 | 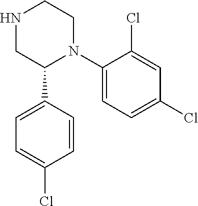 [SMILES: Clc1ccc([C@@H]2CNCCN2c2ccc(Cl)cc2Cl)cc1] | 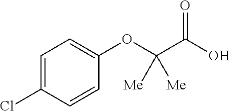 [SMILES: CC(C)(Oc1ccc(Cl)cc1)C(=O)O] | 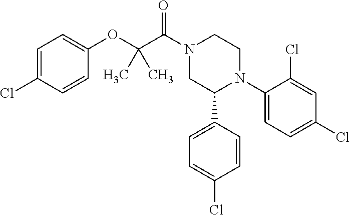 [SMILES: CC(C)(Oc1ccc(Cl)cc1)C(=O)N1CCN(c2ccc(Cl)cc2Cl)[C@H](c2ccc(Cl)cc2)C1] |
| 533 | 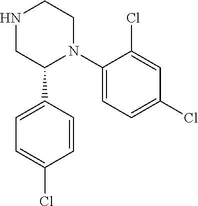 [SMILES: Clc1ccc([C@@H]2CNCCN2c2ccc(Cl)cc2Cl)cc1] | 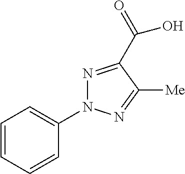 [SMILES: Cc1nn(-c2ccccc2)nc1C(=O)O] | 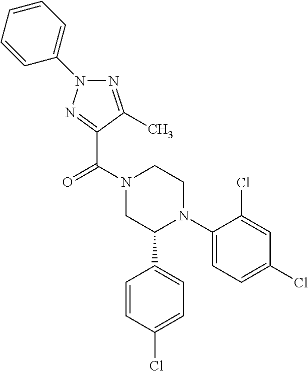 [SMILES: Cc1nn(-c2ccccc2)nc1C(=O)N1CCN(c2ccc(Cl)cc2Cl)[C@H](c2ccc(Cl)cc2)C1] |
| 534 | 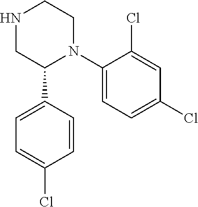 [SMILES: Clc1ccc([C@@H]2CNCCN2c2ccc(Cl)cc2Cl)cc1] | 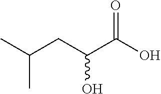 | 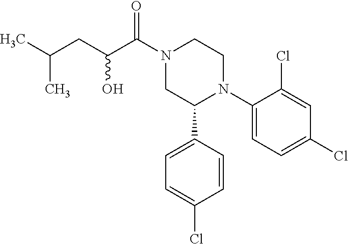 [SMILES: CC(C)CC(O)C(=O)N1CCN(c2ccc(Cl)cc2Cl)[C@H](c2ccc(Cl)cc2)C1] |
| 535 | 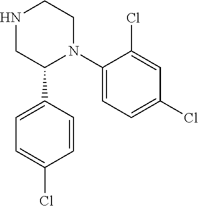 [SMILES: Clc1ccc([C@@H]2CNCCN2c2ccc(Cl)cc2Cl)cc1] | 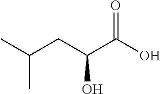 | 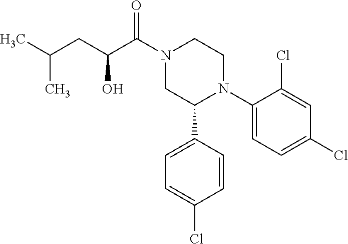 [SMILES: CC(C)C[C@H](O)C(=O)N1CCN(c2ccc(Cl)cc2Cl)[C@H](c2ccc(Cl)cc2)C1] |
| 536 | 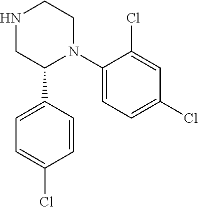 [SMILES: Clc1ccc([C@@H]2CNCCN2c2ccc(Cl)cc2Cl)cc1] | 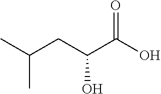 | 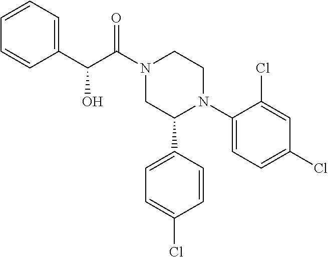 [SMILES: O=C([C@H](O)c1ccccc1)N1CCN(c2ccc(Cl)cc2Cl)[C@H](c2ccc(Cl)cc2)C1] |
| 537 | 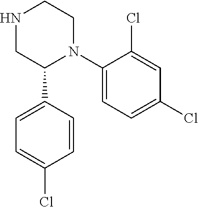 [SMILES: Clc1ccc([C@@H]2CNCCN2c2ccc(Cl)cc2Cl)cc1] | 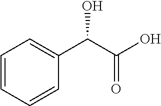 [SMILES: O=C(O)[C@@H](O)c1ccccc1] | 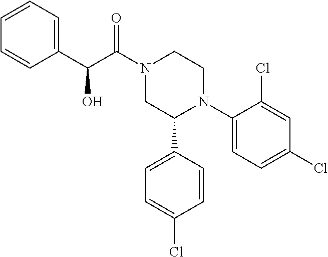 [SMILES: O=C([C@@H](O)c1ccccc1)N1CCN(c2ccc(Cl)cc2Cl)[C@H](c2ccc(Cl)cc2)C1] |
| 538 | 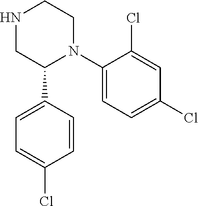 [SMILES: Clc1ccc([C@@H]2CNCCN2c2ccc(Cl)cc2Cl)cc1] | 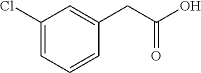 [SMILES: O=C(O)Cc1cccc(Cl)c1] | 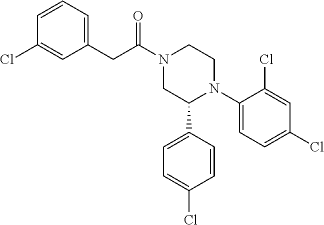 [SMILES: O=C(Cc1cccc(Cl)c1)N1CCN(c2ccc(Cl)cc2Cl)[C@H](c2ccc(Cl)cc2)C1] |
| 539 | 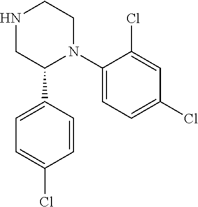 [SMILES: Clc1ccc([C@@H]2CNCCN2c2ccc(Cl)cc2Cl)cc1] | 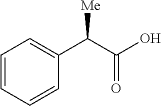 [SMILES: C[C@@H](C(=O)O)c1ccccc1] | 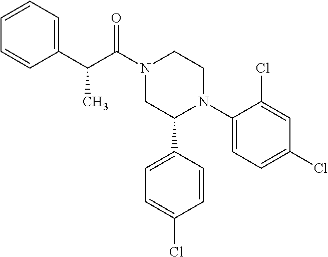 [SMILES: C[C@@H](C(=O)N1CCN(c2ccc(Cl)cc2Cl)[C@H](c2ccc(Cl)cc2)C1)c1ccccc1] |
| 540 | 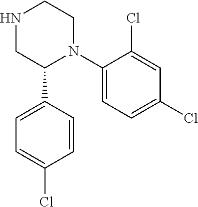 [SMILES: Clc1ccc([C@@H]2CNCCN2c2ccc(Cl)cc2Cl)cc1] | 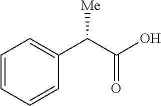 [SMILES: C[C@H](C(=O)O)c1ccccc1] | 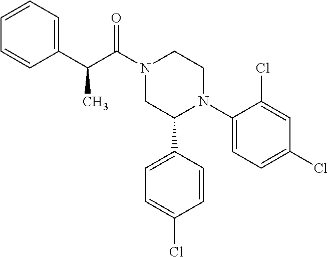 [SMILES: C[C@H](C(=O)N1CCN(c2ccc(Cl)cc2Cl)[C@H](c2ccc(Cl)cc2)C1)c1ccccc1] |
| 541 | 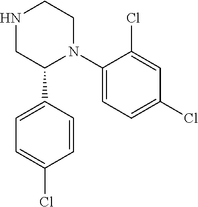 [SMILES: Clc1ccc([C@@H]2CNCCN2c2ccc(Cl)cc2Cl)cc1] | 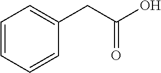 | 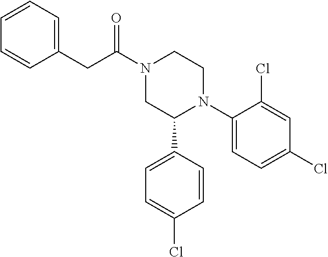 [SMILES: O=C(Cc1ccccc1)N1CCN(c2ccc(Cl)cc2Cl)[C@H](c2ccc(Cl)cc2)C1] |
| 542 | 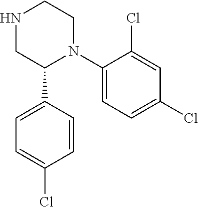 [SMILES: Clc1ccc([C@@H]2CNCCN2c2ccc(Cl)cc2Cl)cc1] | 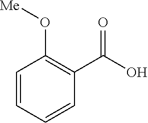 [SMILES: COc1ccccc1C(=O)O] | 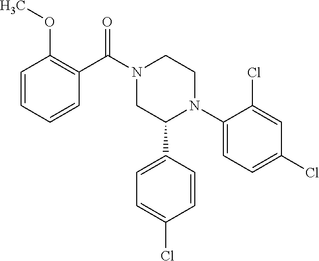 [SMILES: COc1ccccc1C(=O)N1CCN(c2ccc(Cl)cc2Cl)[C@H](c2ccc(Cl)cc2)C1] |
| 543 | 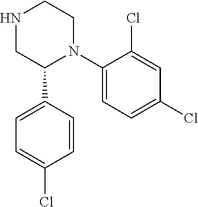 [SMILES: Clc1ccc([C@@H]2CNCCN2c2ccc(Cl)cc2Cl)cc1] | 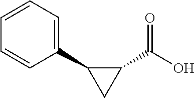 [SMILES: O=C(O)[C@@H]1C[C@H]1c1ccccc1] | 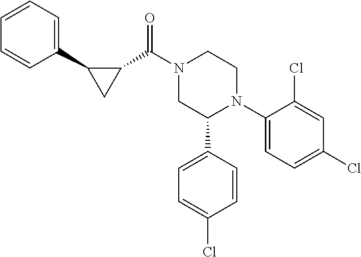 [SMILES: O=C([C@@H]1C[C@H]1c1ccccc1)N1CCN(c2ccc(Cl)cc2Cl)[C@H](c2ccc(Cl)cc2)C1] |
| 544 | 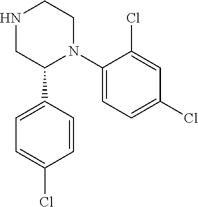 [SMILES: Clc1ccc([C@@H]2CNCCN2c2ccc(Cl)cc2Cl)cc1] | 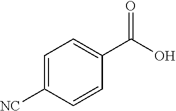 [SMILES: N#Cc1ccc(C(=O)O)cc1] | 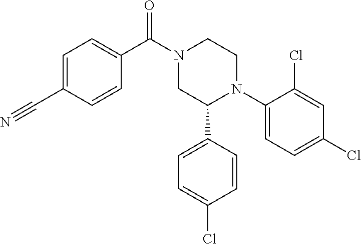 [SMILES: N#Cc1ccc(C(=O)N2CCN(c3ccc(Cl)cc3Cl)[C@H](c3ccc(Cl)cc3)C2)cc1] |
| 545 | 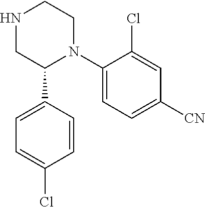 [SMILES: N#Cc1ccc(N2CCNC[C@H]2c2ccc(Cl)cc2)c(Cl)c1] | 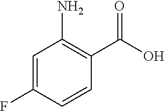 [SMILES: Nc1cc(F)ccc1C(=O)O] | 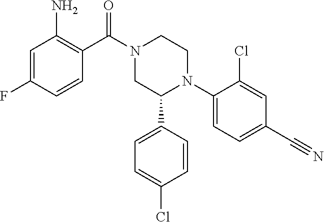 [SMILES: N#Cc1ccc(N2CCN(C(=O)c3ccc(F)cc3N)C[C@H]2c2ccc(Cl)cc2)c(Cl)c1] |
| 546 | 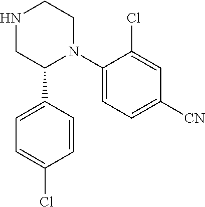 [SMILES: N#Cc1ccc(N2CCNC[C@H]2c2ccc(Cl)cc2)c(Cl)c1] | 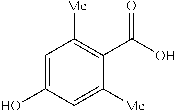 [SMILES: Cc1cc(O)cc(C)c1C(=O)O] | 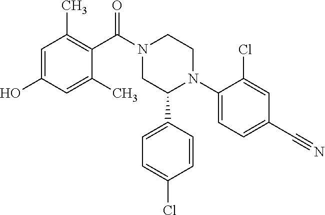 [SMILES: Cc1cc(O)cc(C)c1C(=O)N1CCN(c2ccc(C#N)cc2Cl)[C@H](c2ccc(Cl)cc2)C1] |
| 547 | 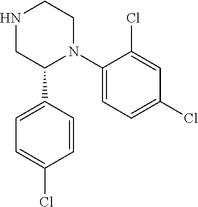 [SMILES: Clc1ccc([C@@H]2CNCCN2c2ccc(Cl)cc2Cl)cc1] | 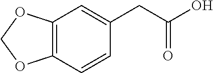 [SMILES: O=C(O)Cc1ccc2c(c1)OCO2] | 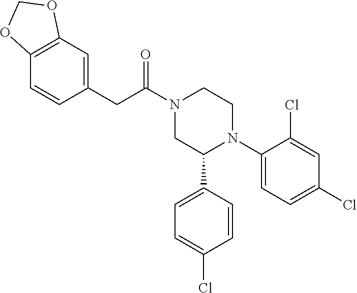 [SMILES: O=C(Cc1ccc2c(c1)OCO2)N1CCN(c2ccc(Cl)cc2Cl)[C@H](c2ccc(Cl)cc2)C1] |
| 548 | 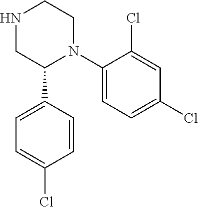 [SMILES: Clc1ccc([C@@H]2CNCCN2c2ccc(Cl)cc2Cl)cc1] | 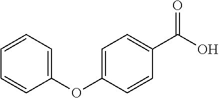 [SMILES: O=C(O)c1ccc(Oc2ccccc2)cc1] | 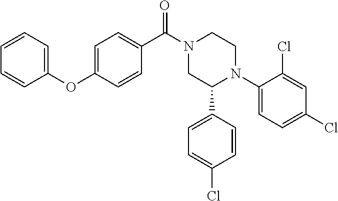 [SMILES: O=C(c1ccc(Oc2ccccc2)cc1)N1CCN(c2ccc(Cl)cc2Cl)[C@H](c2ccc(Cl)cc2)C1] |
| 549 | 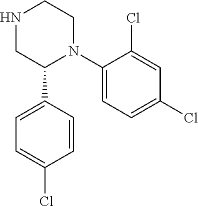 [SMILES: Clc1ccc([C@@H]2CNCCN2c2ccc(Cl)cc2Cl)cc1] | 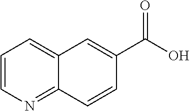 [SMILES: O=C(O)c1ccc2ncccc2c1] | 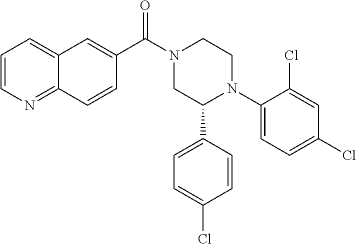 [SMILES: O=C(c1ccc2ncccc2c1)N1CCN(c2ccc(Cl)cc2Cl)[C@H](c2ccc(Cl)cc2)C1] |
| 550 | 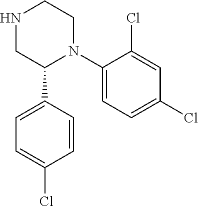 [SMILES: Clc1ccc([C@@H]2CNCCN2c2ccc(Cl)cc2Cl)cc1] | 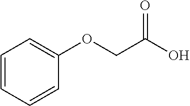 [SMILES: O=C(O)COc1ccccc1] | 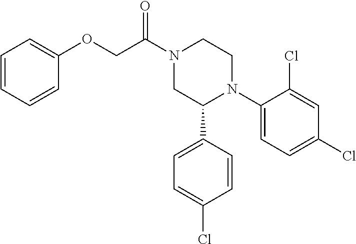 [SMILES: O=C(COc1ccccc1)N1CCN(c2ccc(Cl)cc2Cl)[C@H](c2ccc(Cl)cc2)C1] |
| 551 | 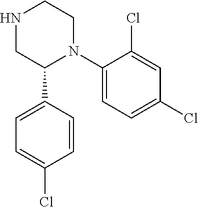 [SMILES: Clc1ccc([C@@H]2CNCCN2c2ccc(Cl)cc2Cl)cc1] | 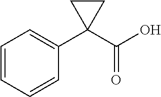 [SMILES: O=C(O)C1(c2ccccc2)CC1] | 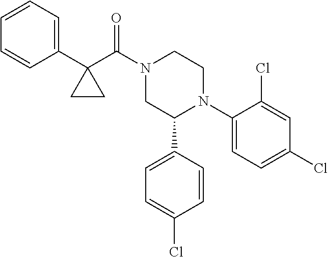 [SMILES: O=C(N1CCN(c2ccc(Cl)cc2Cl)[C@H](c2ccc(Cl)cc2)C1)C1(c2ccccc2)CC1] |
| 552 | 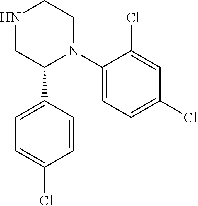 [SMILES: Clc1ccc([C@@H]2CNCCN2c2ccc(Cl)cc2Cl)cc1] | 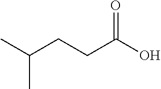 | 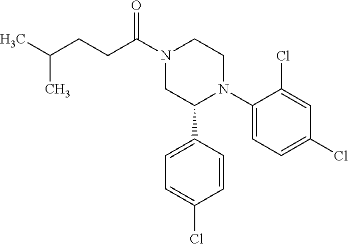 [SMILES: CC(C)CCC(=O)N1CCN(c2ccc(Cl)cc2Cl)[C@H](c2ccc(Cl)cc2)C1] |
| 553 | 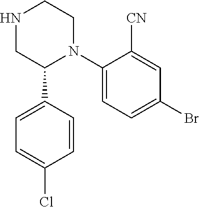 [SMILES: N#Cc1cc(Br)ccc1N1CCNC[C@H]1c1ccc(Cl)cc1] | 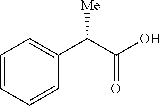 [SMILES: C[C@H](C(=O)O)c1ccccc1] | 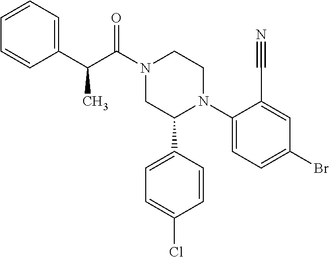 [SMILES: C[C@H](C(=O)N1CCN(c2ccc(Br)cc2C#N)[C@H](c2ccc(Cl)cc2)C1)c1ccccc1] |
| 554 | 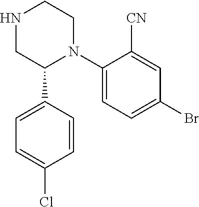 [SMILES: N#Cc1cc(Br)ccc1N1CCNC[C@H]1c1ccc(Cl)cc1] | 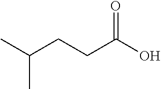 | 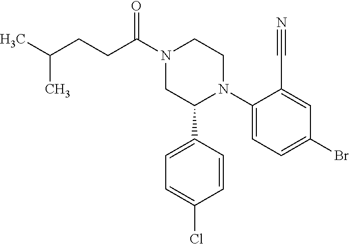 [SMILES: CC(C)CCC(=O)N1CCN(c2ccc(Br)cc2C#N)[C@H](c2ccc(Cl)cc2)C1] |
| 555 | 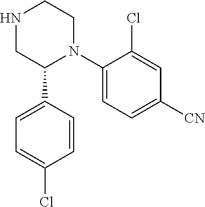 [SMILES: N#Cc1ccc(N2CCNC[C@H]2c2ccc(Cl)cc2)c(Cl)c1] | 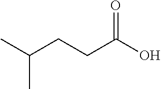 | 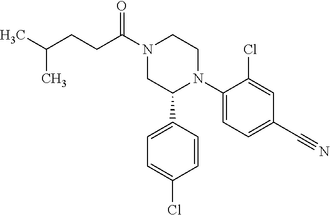 [SMILES: CC(C)CCC(=O)N1CCN(c2ccc(C#N)cc2Cl)[C@H](c2ccc(Cl)cc2)C1] |
| 556 | 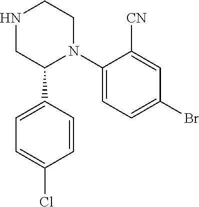 [SMILES: N#Cc1cc(Br)ccc1N1CCNC[C@H]1c1ccc(Cl)cc1] | 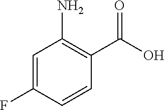 [SMILES: Nc1cc(F)ccc1C(=O)O] | 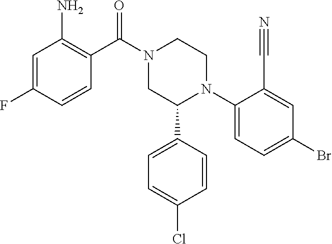 [SMILES: N#Cc1cc(Br)ccc1N1CCN(C(=O)c2ccc(F)cc2N)C[C@H]1c1ccc(Cl)cc1] |
| 557 | 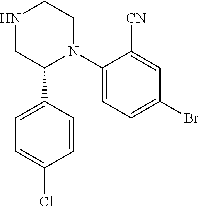 [SMILES: N#Cc1cc(Br)ccc1N1CCNC[C@H]1c1ccc(Cl)cc1] | 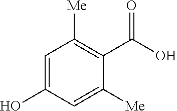 [SMILES: Cc1cc(O)cc(C)c1C(=O)O] | 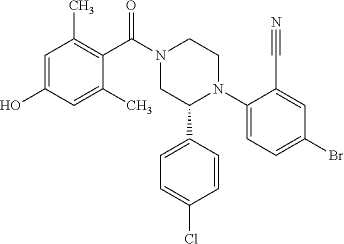 [SMILES: Cc1cc(O)cc(C)c1C(=O)N1CCN(c2ccc(Br)cc2C#N)[C@H](c2ccc(Cl)cc2)C1] |
| 558 | 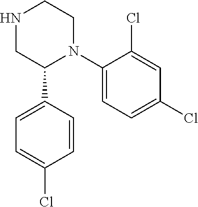 [SMILES: Clc1ccc([C@@H]2CNCCN2c2ccc(Cl)cc2Cl)cc1] | 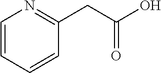 | 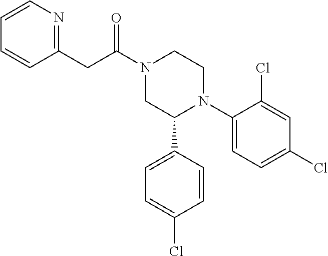 [SMILES: O=C(Cc1ccccn1)N1CCN(c2ccc(Cl)cc2Cl)[C@H](c2ccc(Cl)cc2)C1] |
| 559 | 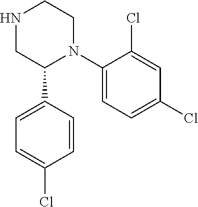 [SMILES: Clc1ccc([C@@H]2CNCCN2c2ccc(Cl)cc2Cl)cc1] | 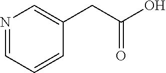 | 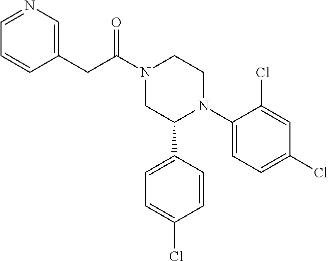 [SMILES: O=C(Cc1cccnc1)N1CCN(c2ccc(Cl)cc2Cl)[C@H](c2ccc(Cl)cc2)C1] |
| 560 | 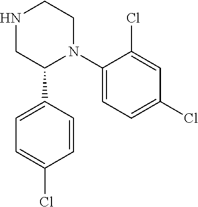 [SMILES: Clc1ccc([C@@H]2CNCCN2c2ccc(Cl)cc2Cl)cc1] | 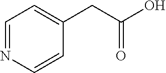 | 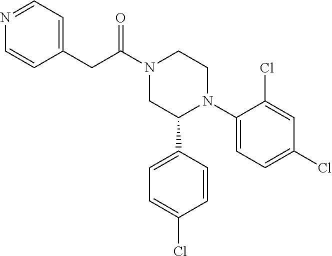 [SMILES: O=C(Cc1ccncc1)N1CCN(c2ccc(Cl)cc2Cl)[C@H](c2ccc(Cl)cc2)C1] |
| 561 | 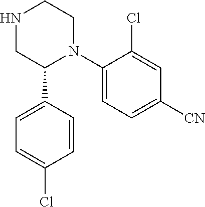 [SMILES: N#Cc1ccc(N2CCNC[C@H]2c2ccc(Cl)cc2)c(Cl)c1] | 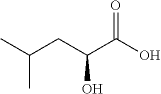 | 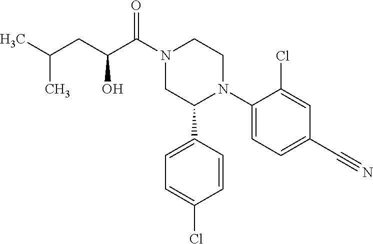 [SMILES: CC(C)C[C@H](O)C(=O)N1CCN(c2ccc(C#N)cc2Cl)[C@H](c2ccc(Cl)cc2)C1] |
| 562 | 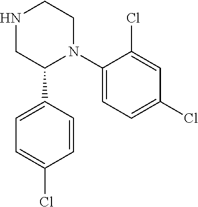 [SMILES: Clc1ccc([C@@H]2CNCCN2c2ccc(Cl)cc2Cl)cc1] | 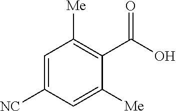 [SMILES: Cc1cc(C#N)cc(C)c1C(=O)O] | 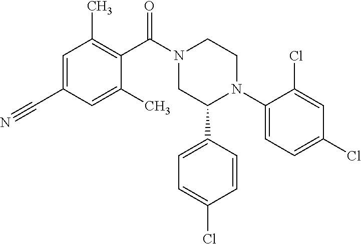 [SMILES: Cc1cc(C#N)cc(C)c1C(=O)N1CCN(c2ccc(Cl)cc2Cl)[C@H](c2ccc(Cl)cc2)C1] |
| 563 | 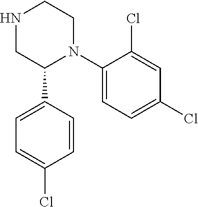 [SMILES: Clc1ccc([C@@H]2CNCCN2c2ccc(Cl)cc2Cl)cc1] | 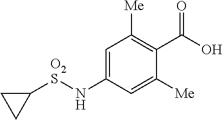 [SMILES: Cc1cc(NS(=O)(=O)C2CC2)cc(C)c1C(=O)O] | 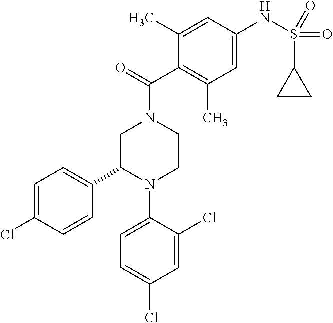 [SMILES: Cc1cc(NS(=O)(=O)C2CC2)cc(C)c1C(=O)N1CCN(c2ccc(Cl)cc2Cl)[C@H](c2ccc(Cl)cc2)C1] |
| 564 | 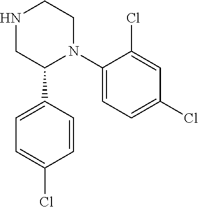 [SMILES: Clc1ccc([C@@H]2CNCCN2c2ccc(Cl)cc2Cl)cc1] | 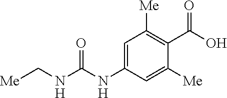 [SMILES: CCNC(=O)Nc1cc(C)c(C(=O)O)c(C)c1] | 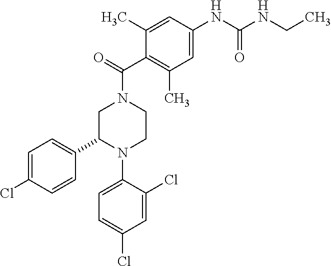 [SMILES: CCNC(=O)Nc1cc(C)c(C(=O)N2CCN(c3ccc(Cl)cc3Cl)[C@H](c3ccc(Cl)cc3)C2)c(C)c1] |
| 565 | 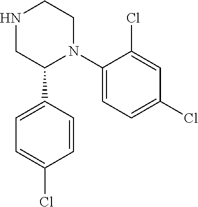 [SMILES: Clc1ccc([C@@H]2CNCCN2c2ccc(Cl)cc2Cl)cc1] | 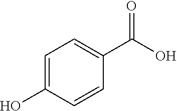 | 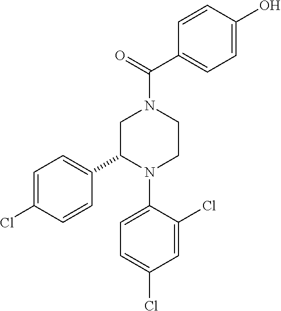 [SMILES: O=C(c1ccc(O)cc1)N1CCN(c2ccc(Cl)cc2Cl)[C@H](c2ccc(Cl)cc2)C1] |
| 566 | 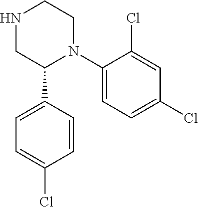 [SMILES: Clc1ccc([C@@H]2CNCCN2c2ccc(Cl)cc2Cl)cc1] | 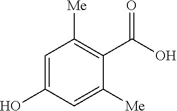 [SMILES: Cc1cc(O)cc(C)c1C(=O)O] | 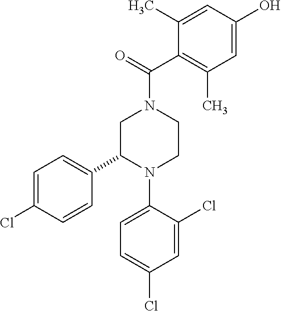 [SMILES: Cc1cc(O)cc(C)c1C(=O)N1CCN(c2ccc(Cl)cc2Cl)[C@H](c2ccc(Cl)cc2)C1] |
| 567 | 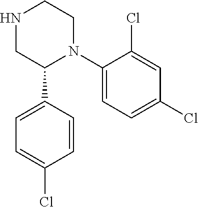 [SMILES: Clc1ccc([C@@H]2CNCCN2c2ccc(Cl)cc2Cl)cc1] | 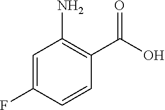 [SMILES: Nc1cc(F)ccc1C(=O)O] | 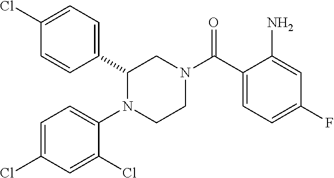 |
| 568 | 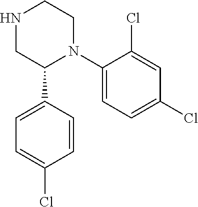 [SMILES: Clc1ccc([C@@H]2CNCCN2c2ccc(Cl)cc2Cl)cc1] | 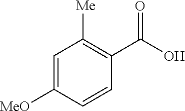 [SMILES: COc1ccc(C(=O)O)c(C)c1] | 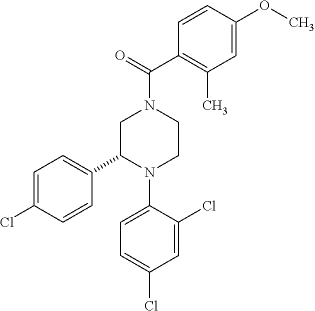 [SMILES: COc1ccc(C(=O)N2CCN(c3ccc(Cl)cc3Cl)[C@H](c3ccc(Cl)cc3)C2)c(C)c1] |
| 569 | 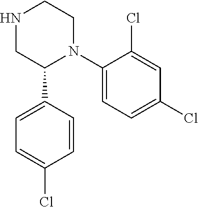 [SMILES: Clc1ccc([C@@H]2CNCCN2c2ccc(Cl)cc2Cl)cc1] | 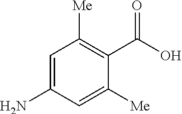 [SMILES: Cc1cc(N)cc(C)c1C(=O)O] | 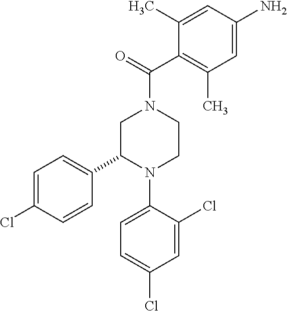 [SMILES: Cc1cc(N)cc(C)c1C(=O)N1CCN(c2ccc(Cl)cc2Cl)[C@H](c2ccc(Cl)cc2)C1] |
| 570 | 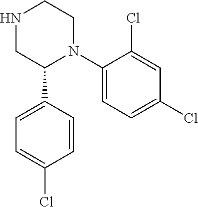 [SMILES: Clc1ccc([C@@H]2CNCCN2c2ccc(Cl)cc2Cl)cc1] | 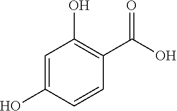 [SMILES: O=C(O)c1ccc(O)cc1O] | 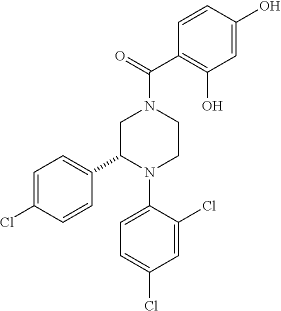 [SMILES: O=C(c1ccc(O)cc1O)N1CCN(c2ccc(Cl)cc2Cl)[C@H](c2ccc(Cl)cc2)C1] |

### Preparation of Example 571

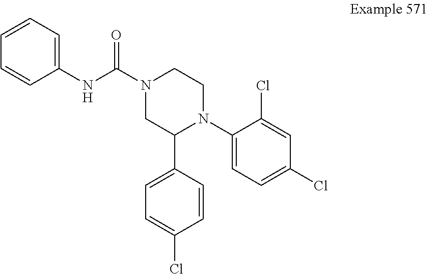 [SMILES: O=C(Nc1ccccc1)N1CCN(c2ccc(Cl)cc2Cl)C(c2ccc(Cl)cc2)C1]

Example 571 was prepared by the reaction of phenyl isocyanate with Example 1 using procedures similar to those used to prepare Examples 7, Scheme 1.

### Preparation of Examples 572-685

Examples 572-685 were prepared by the reaction of piperazine Example 1 and the carboxylic acid shown in Table XX, using the procedure of Scheme 5.

TABLE XX

| Example # | Carboxylic Acid | Example Structure |
|---|---|---|
| 572 | 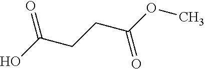 | 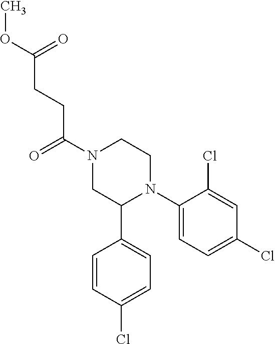 [SMILES: COC(=O)CCC(=O)N1CCN(c2ccc(Cl)cc2Cl)C(c2ccc(Cl)cc2)C1] |
| 573 | 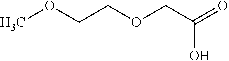 | 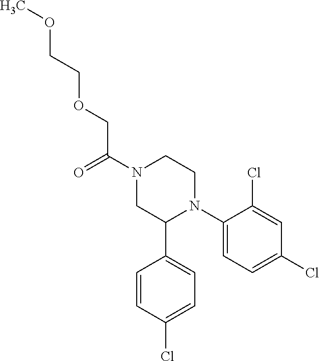 [SMILES: COCCOCC(=O)N1CCN(c2ccc(Cl)cc2Cl)C(c2ccc(Cl)cc2)C1] |
| 574 | 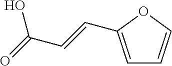 | 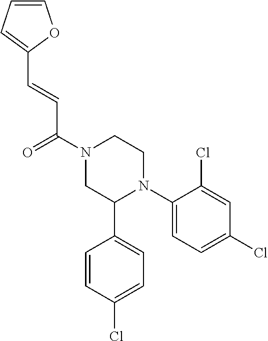 [SMILES: O=C(/C=C/c1ccco1)N1CCN(c2ccc(Cl)cc2Cl)C(c2ccc(Cl)cc2)C1] |
| 575 | 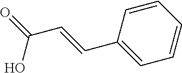 [SMILES: O=C(O)/C=C/c1ccccc1] | 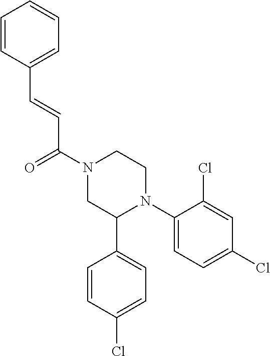 [SMILES: O=C(/C=C/c1ccccc1)N1CCN(c2ccc(Cl)cc2Cl)C(c2ccc(Cl)cc2)C1] |
| 576 | 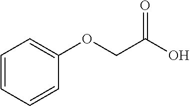 [SMILES: O=C(O)COc1ccccc1] | 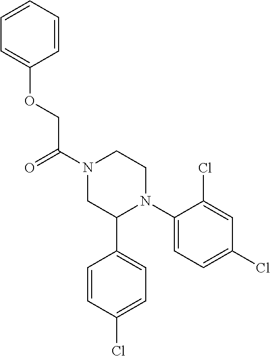 [SMILES: O=C(COc1ccccc1)N1CCN(c2ccc(Cl)cc2Cl)C(c2ccc(Cl)cc2)C1] |
| 577 | 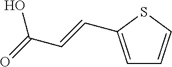 | 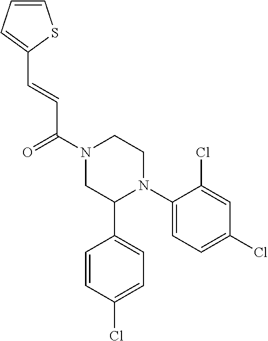 [SMILES: O=C(/C=C/c1cccs1)N1CCN(c2ccc(Cl)cc2Cl)C(c2ccc(Cl)cc2)C1] |
| 578 | 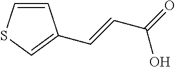 | 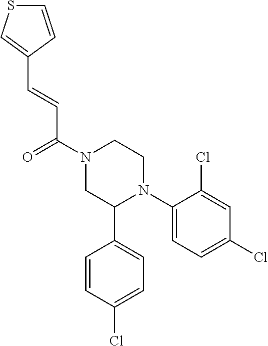 [SMILES: O=C(/C=C/c1ccsc1)N1CCN(c2ccc(Cl)cc2Cl)C(c2ccc(Cl)cc2)C1] |
| 579 | 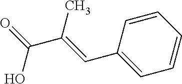 | 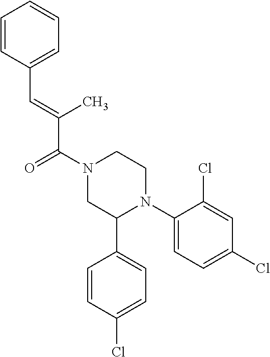 [SMILES: C/C(=C\c1ccccc1)C(=O)N1CCN(c2ccc(Cl)cc2Cl)C(c2ccc(Cl)cc2)C1] |
| 580 | 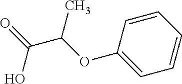 | 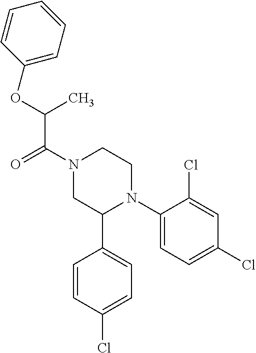 [SMILES: CC(Oc1ccccc1)C(=O)N1CCN(c2ccc(Cl)cc2Cl)C(c2ccc(Cl)cc2)C1] |
| 581 | 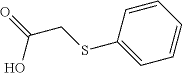 [SMILES: O=C(O)CSc1ccccc1] | 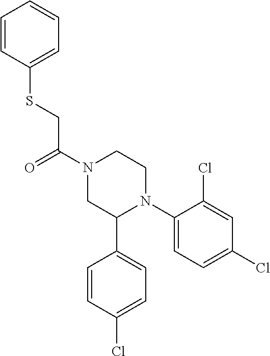 [SMILES: O=C(CSc1ccccc1)N1CCN(c2ccc(Cl)cc2Cl)C(c2ccc(Cl)cc2)C1] |
| 582 | 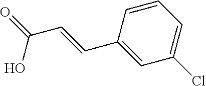 [SMILES: O=C(O)/C=C/c1cccc(Cl)c1] | 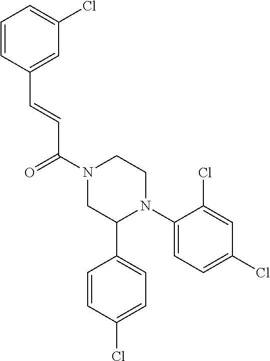 [SMILES: O=C(/C=C/c1cccc(Cl)c1)N1CCN(c2ccc(Cl)cc2Cl)C(c2ccc(Cl)cc2)C1] |
| 583 | 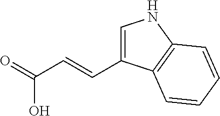 [SMILES: O=C(O)/C=C/c1c[nH]c2ccccc12] | 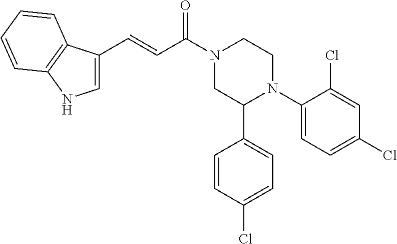 [SMILES: O=C(/C=C/c1c[nH]c2ccccc12)N1CCN(c2ccc(Cl)cc2Cl)C(c2ccc(Cl)cc2)C1] |
| 584 | 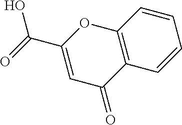 [SMILES: O=C(O)c1cc(=O)c2ccccc2o1] | 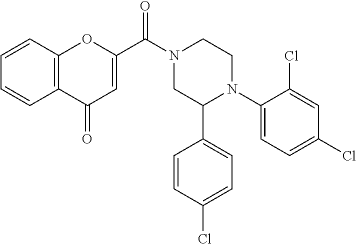 [SMILES: O=C(c1cc(=O)c2ccccc2o1)N1CCN(c2ccc(Cl)cc2Cl)C(c2ccc(Cl)cc2)C1] |
| 585 | 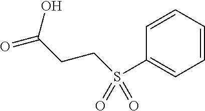 [SMILES: O=C(O)CCS(=O)(=O)c1ccccc1] | 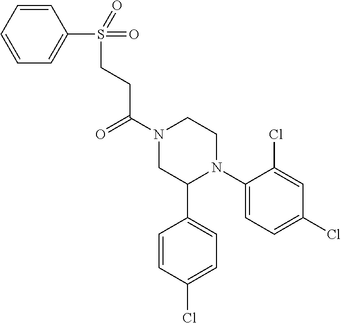 [SMILES: O=C(CCS(=O)(=O)c1ccccc1)N1CCN(c2ccc(Cl)cc2Cl)C(c2ccc(Cl)cc2)C1] |
| 586 | 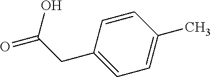 | 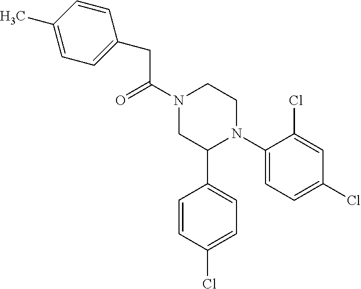 [SMILES: Cc1ccc(CC(=O)N2CCN(c3ccc(Cl)cc3Cl)C(c3ccc(Cl)cc3)C2)cc1] |
| 587 | 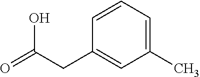 | 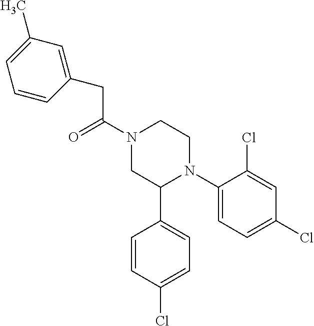 [SMILES: Cc1cccc(CC(=O)N2CCN(c3ccc(Cl)cc3Cl)C(c3ccc(Cl)cc3)C2)c1] |
| 588 | 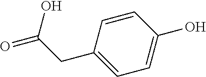 [SMILES: O=C(O)Cc1ccc(O)cc1] | 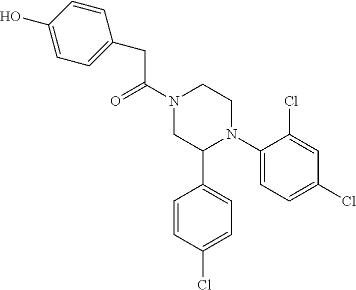 [SMILES: O=C(Cc1ccc(O)cc1)N1CCN(c2ccc(Cl)cc2Cl)C(c2ccc(Cl)cc2)C1] |
| 589 | 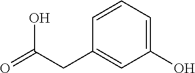 [SMILES: O=C(O)Cc1cccc(O)c1] | 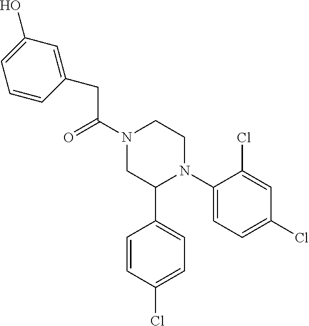 [SMILES: O=C(Cc1cccc(O)c1)N1CCN(c2ccc(Cl)cc2Cl)C(c2ccc(Cl)cc2)C1] |
| 590 | 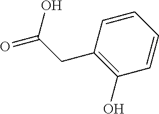 [SMILES: O=C(O)Cc1ccccc1O] | 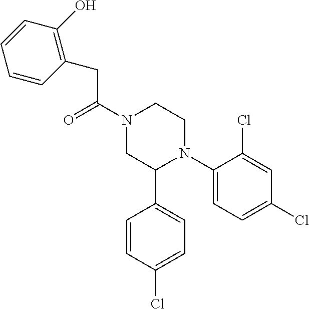 [SMILES: O=C(Cc1ccccc1O)N1CCN(c2ccc(Cl)cc2Cl)C(c2ccc(Cl)cc2)C1] |
| 591 | 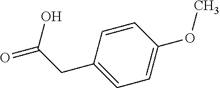 | 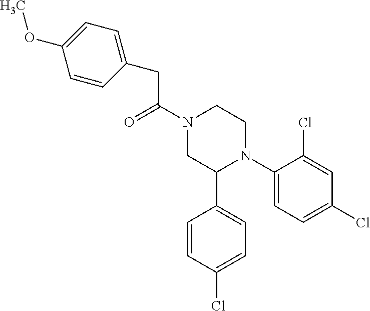 [SMILES: COc1ccc(CC(=O)N2CCN(c3ccc(Cl)cc3Cl)C(c3ccc(Cl)cc3)C2)cc1] |
| 592 | 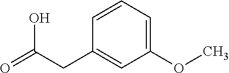 | 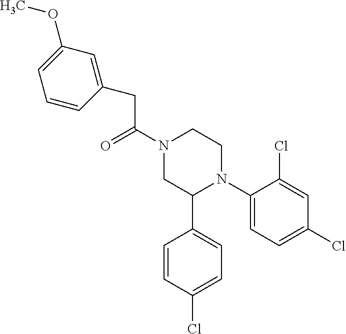 [SMILES: COc1cccc(CC(=O)N2CCN(c3ccc(Cl)cc3Cl)C(c3ccc(Cl)cc3)C2)c1] |
| 593 | 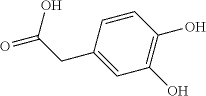 [SMILES: O=C(O)Cc1ccc(O)c(O)c1] | 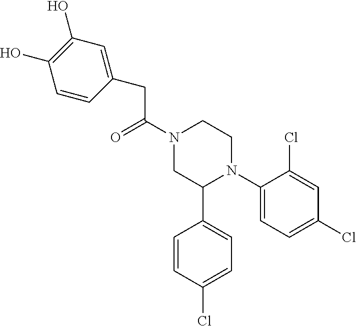 [SMILES: O=C(Cc1ccc(O)c(O)c1)N1CCN(c2ccc(Cl)cc2Cl)C(c2ccc(Cl)cc2)C1] |
| 594 | 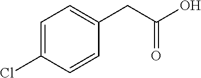 [SMILES: O=C(O)Cc1ccc(Cl)cc1] | 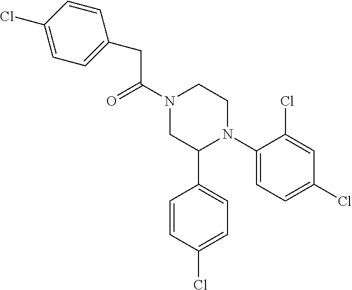 [SMILES: O=C(Cc1ccc(Cl)cc1)N1CCN(c2ccc(Cl)cc2Cl)C(c2ccc(Cl)cc2)C1] |
| 595 | 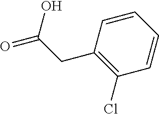 [SMILES: O=C(O)Cc1ccccc1Cl] | 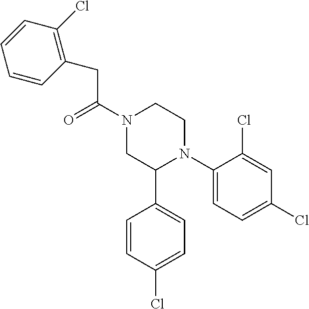 [SMILES: O=C(Cc1ccccc1Cl)N1CCN(c2ccc(Cl)cc2Cl)C(c2ccc(Cl)cc2)C1] |
| 596 | 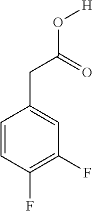 [SMILES: O=C(O)Cc1ccc(F)c(F)c1] | 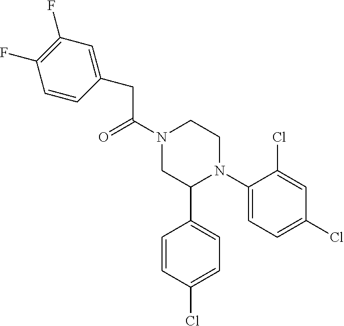 [SMILES: O=C(Cc1ccc(F)c(F)c1)N1CCN(c2ccc(Cl)cc2Cl)C(c2ccc(Cl)cc2)C1] |
| 597 | 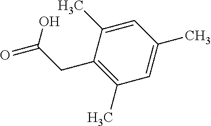 | 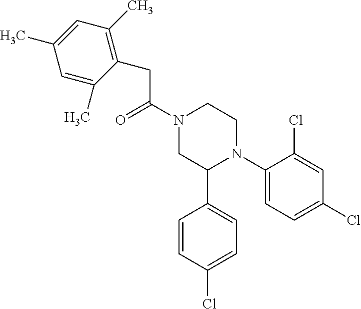 [SMILES: Cc1cc(C)c(CC(=O)N2CCN(c3ccc(Cl)cc3Cl)C(c3ccc(Cl)cc3)C2)c(C)c1] |
| 598 | 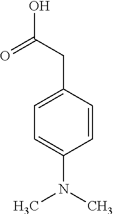 | 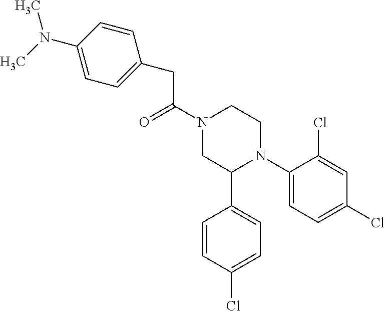 [SMILES: CN(C)c1ccc(CC(=O)N2CCN(c3ccc(Cl)cc3Cl)C(c3ccc(Cl)cc3)C2)cc1] |
| 599 | 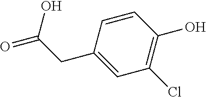 [SMILES: O=C(O)Cc1ccc(O)c(Cl)c1] | 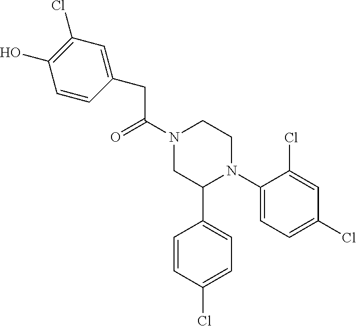 [SMILES: O=C(Cc1ccc(O)c(Cl)c1)N1CCN(c2ccc(Cl)cc2Cl)C(c2ccc(Cl)cc2)C1] |
| 600 | 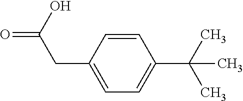 | 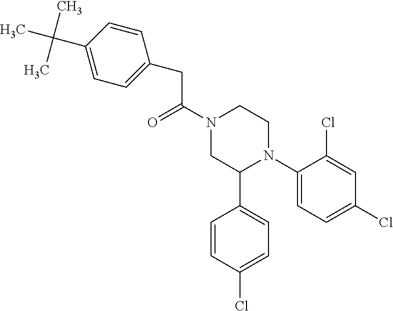 [SMILES: CC(C)(C)c1ccc(CC(=O)N2CCN(c3ccc(Cl)cc3Cl)C(c3ccc(Cl)cc3)C2)cc1] |
| 601 | 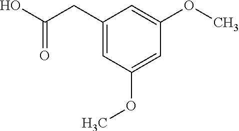 | 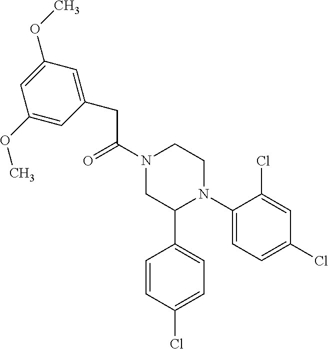 [SMILES: COc1cc(CC(=O)N2CCN(c3ccc(Cl)cc3Cl)C(c3ccc(Cl)cc3)C2)cc(OC)c1] |
| 602 | 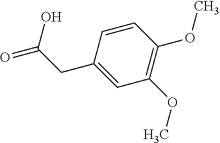 | 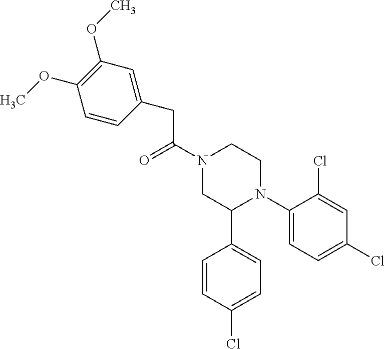 [SMILES: COc1ccc(CC(=O)N2CCN(c3ccc(Cl)cc3Cl)C(c3ccc(Cl)cc3)C2)cc1OC] |
| 603 | 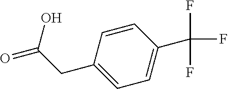 [SMILES: O=C(O)Cc1ccc(C(F)(F)F)cc1] | 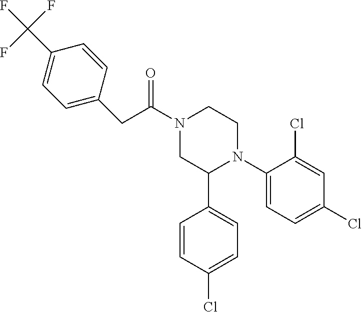 [SMILES: O=C(Cc1ccc(C(F)(F)F)cc1)N1CCN(c2ccc(Cl)cc2Cl)C(c2ccc(Cl)cc2)C1] |
| 604 | 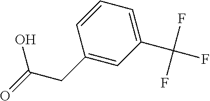 [SMILES: O=C(O)Cc1cccc(C(F)(F)F)c1] | 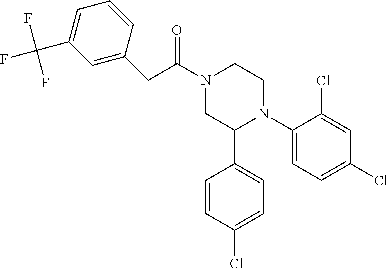 [SMILES: O=C(Cc1cccc(C(F)(F)F)c1)N1CCN(c2ccc(Cl)cc2Cl)C(c2ccc(Cl)cc2)C1] |
| 605 | 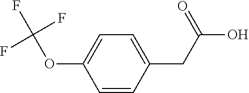 [SMILES: O=C(O)Cc1ccc(OC(F)(F)F)cc1] | 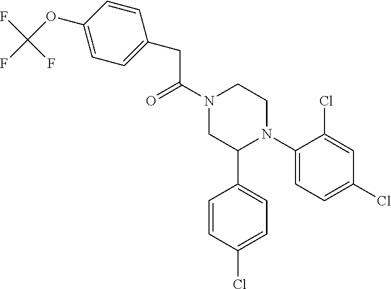 [SMILES: O=C(Cc1ccc(OC(F)(F)F)cc1)N1CCN(c2ccc(Cl)cc2Cl)C(c2ccc(Cl)cc2)C1] |
| 606 | 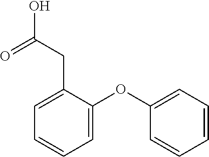 [SMILES: O=C(O)Cc1ccccc1Oc1ccccc1] | 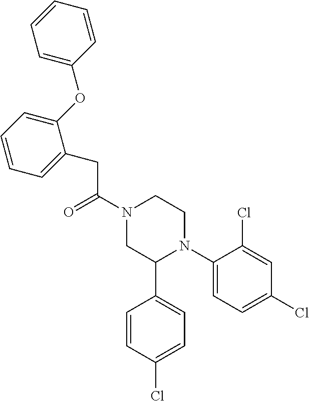 [SMILES: O=C(Cc1ccccc1Oc1ccccc1)N1CCN(c2ccc(Cl)cc2Cl)C(c2ccc(Cl)cc2)C1] |
| 607 | 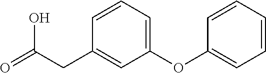 [SMILES: O=C(O)Cc1cccc(Oc2ccccc2)c1] | 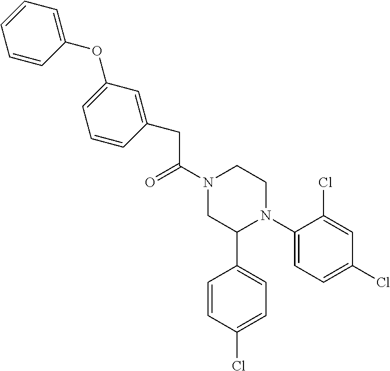 [SMILES: O=C(Cc1cccc(Oc2ccccc2)c1)N1CCN(c2ccc(Cl)cc2Cl)C(c2ccc(Cl)cc2)C1] |
| 608 | 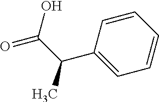 | 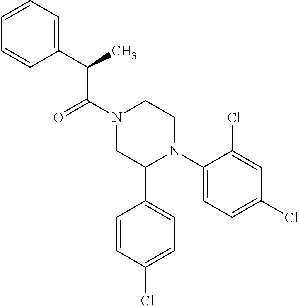 [SMILES: C[C@@H](C(=O)N1CCN(c2ccc(Cl)cc2Cl)C(c2ccc(Cl)cc2)C1)c1ccccc1] |
| 609 | 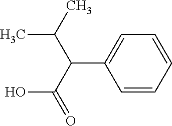 | 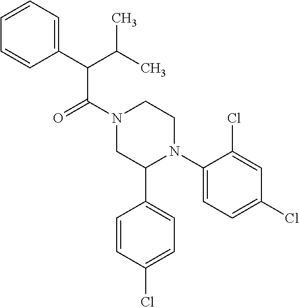 [SMILES: CC(C)C(C(=O)N1CCN(c2ccc(Cl)cc2Cl)C(c2ccc(Cl)cc2)C1)c1ccccc1] |
| 610 | 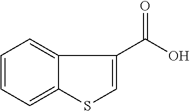 [SMILES: O=C(O)c1csc2ccccc12] | 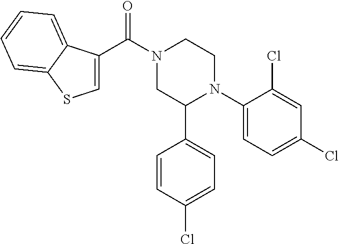 [SMILES: O=C(c1csc2ccccc12)N1CCN(c2ccc(Cl)cc2Cl)C(c2ccc(Cl)cc2)C1] |
| 611 | 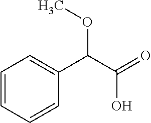 | 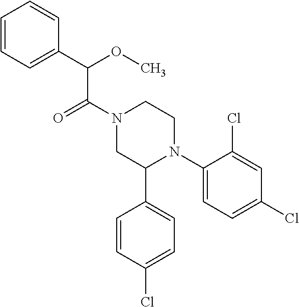 [SMILES: COC(C(=O)N1CCN(c2ccc(Cl)cc2Cl)C(c2ccc(Cl)cc2)C1)c1ccccc1] |
| 612 | 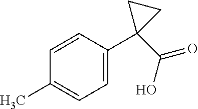 | 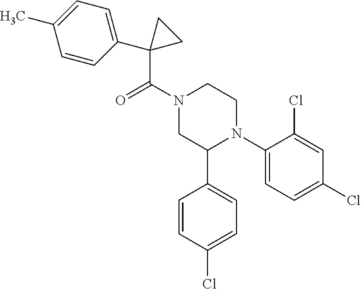 [SMILES: Cc1ccc(C2(C(=O)N3CCN(c4ccc(Cl)cc4Cl)C(c4ccc(Cl)cc4)C3)CC2)cc1] |
| 613 | 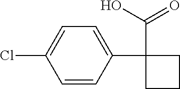 [SMILES: O=C(O)C1(c2ccc(Cl)cc2)CCC1] | 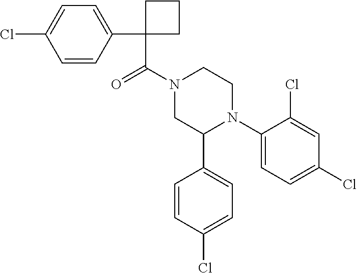 [SMILES: O=C(N1CCN(c2ccc(Cl)cc2Cl)C(c2ccc(Cl)cc2)C1)C1(c2ccc(Cl)cc2)CCC1] |
| 614 | 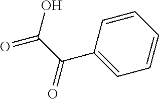 [SMILES: O=C(O)C(=O)c1ccccc1] | 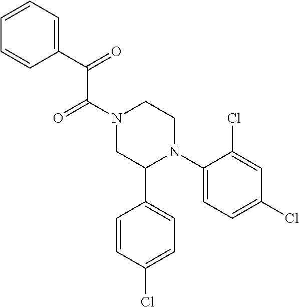 [SMILES: O=C(C(=O)N1CCN(c2ccc(Cl)cc2Cl)C(c2ccc(Cl)cc2)C1)c1ccccc1] |
| 615 | 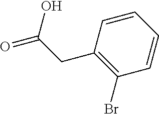 [SMILES: O=C(O)Cc1ccccc1Br] | 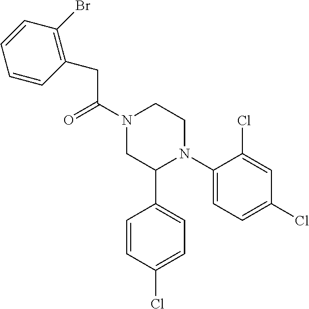 [SMILES: O=C(Cc1ccccc1Br)N1CCN(c2ccc(Cl)cc2Cl)C(c2ccc(Cl)cc2)C1] |
| 616 | 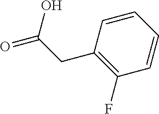 [SMILES: O=C(O)Cc1ccccc1F] | 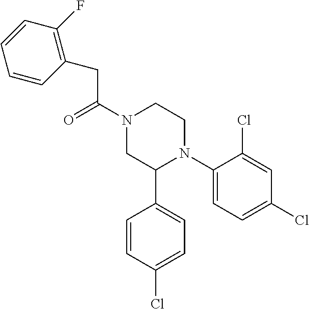 [SMILES: O=C(Cc1ccccc1F)N1CCN(c2ccc(Cl)cc2Cl)C(c2ccc(Cl)cc2)C1] |
| 617 | 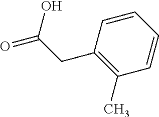 | 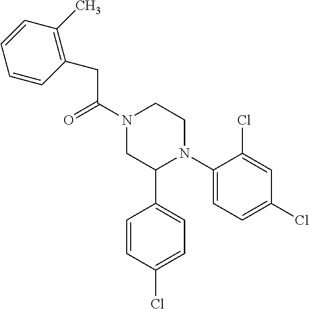 [SMILES: Cc1ccccc1CC(=O)N1CCN(c2ccc(Cl)cc2Cl)C(c2ccc(Cl)cc2)C1] |
| 618 | 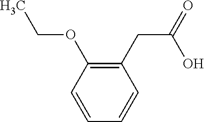 | 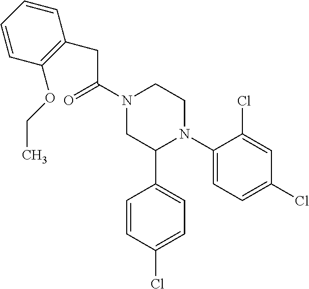 [SMILES: CCOc1ccccc1CC(=O)N1CCN(c2ccc(Cl)cc2Cl)C(c2ccc(Cl)cc2)C1] |
| 619 | 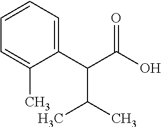 | 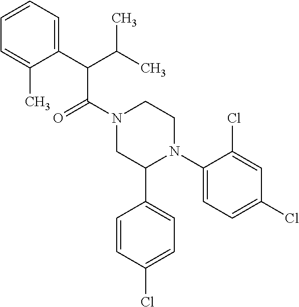 [SMILES: Cc1ccccc1C(C(=O)N1CCN(c2ccc(Cl)cc2Cl)C(c2ccc(Cl)cc2)C1)C(C)C] |
| 620 | 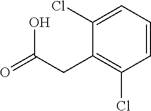 [SMILES: O=C(O)Cc1c(Cl)cccc1Cl] | 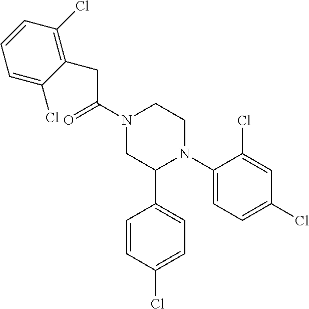 [SMILES: O=C(Cc1c(Cl)cccc1Cl)N1CCN(c2ccc(Cl)cc2Cl)C(c2ccc(Cl)cc2)C1] |
| 621 | 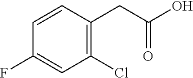 [SMILES: O=C(O)Cc1ccc(F)cc1Cl] | 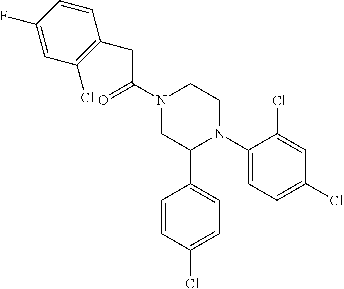 [SMILES: O=C(Cc1ccc(F)cc1Cl)N1CCN(c2ccc(Cl)cc2Cl)C(c2ccc(Cl)cc2)C1] |
| 622 | 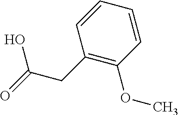 | 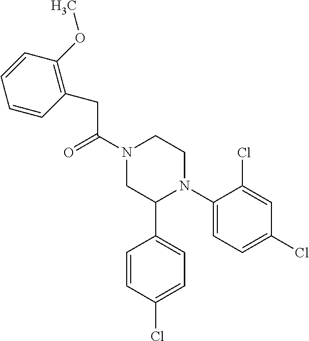 [SMILES: COc1ccccc1CC(=O)N1CCN(c2ccc(Cl)cc2Cl)C(c2ccc(Cl)cc2)C1] |
| 623 | 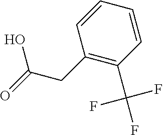 [SMILES: O=C(O)Cc1ccccc1C(F)(F)F] | 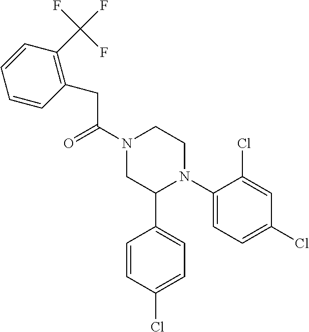 [SMILES: O=C(Cc1ccccc1C(F)(F)F)N1CCN(c2ccc(Cl)cc2Cl)C(c2ccc(Cl)cc2)C1] |
| 624 | 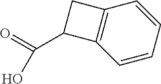 [SMILES: O=C(O)C1Cc2ccccc21] | 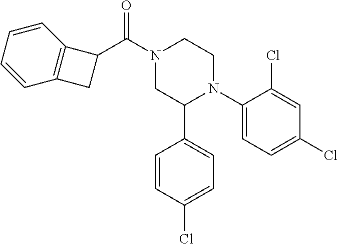 [SMILES: O=C(C1Cc2ccccc21)N1CCN(c2ccc(Cl)cc2Cl)C(c2ccc(Cl)cc2)C1] |
| 625 | 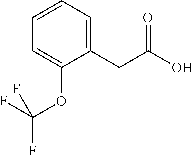 [SMILES: O=C(O)Cc1ccccc1OC(F)(F)F] | 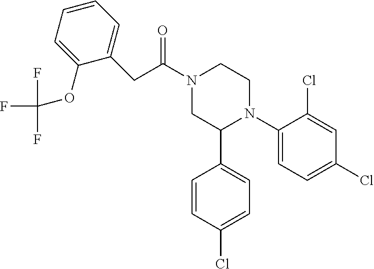 [SMILES: O=C(Cc1ccccc1OC(F)(F)F)N1CCN(c2ccc(Cl)cc2Cl)C(c2ccc(Cl)cc2)C1] |
| 626 | 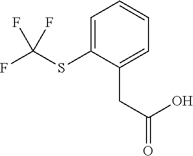 [SMILES: O=C(O)Cc1ccccc1SC(F)(F)F] | 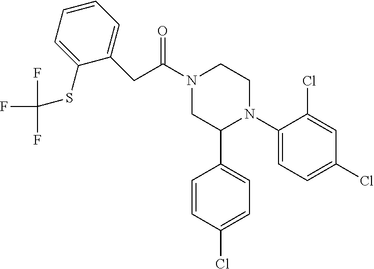 [SMILES: O=C(Cc1ccccc1SC(F)(F)F)N1CCN(c2ccc(Cl)cc2Cl)C(c2ccc(Cl)cc2)C1] |
| 627 | 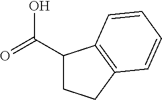 [SMILES: O=C(O)C1CCc2ccccc21] | 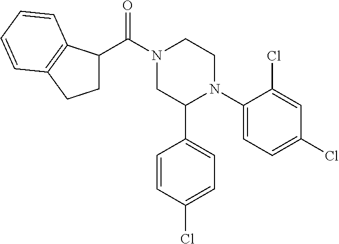 [SMILES: O=C(C1CCc2ccccc21)N1CCN(c2ccc(Cl)cc2Cl)C(c2ccc(Cl)cc2)C1] |
| 628 | 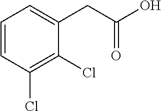 [SMILES: O=C(O)Cc1cccc(Cl)c1Cl] | 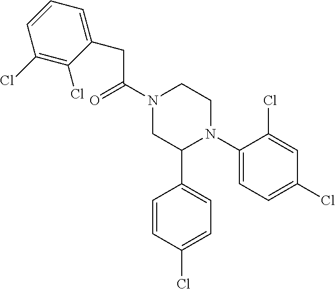 [SMILES: O=C(Cc1cccc(Cl)c1Cl)N1CCN(c2ccc(Cl)cc2Cl)C(c2ccc(Cl)cc2)C1] |
| 629 | 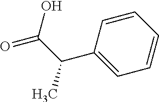 | 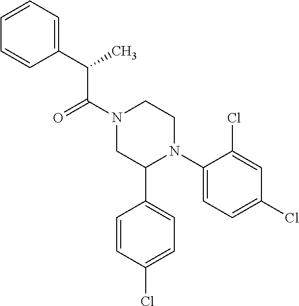 [SMILES: C[C@H](C(=O)N1CCN(c2ccc(Cl)cc2Cl)C(c2ccc(Cl)cc2)C1)c1ccccc1] |
| 630 | 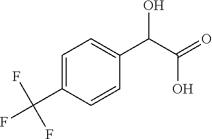 [SMILES: O=C(O)C(O)c1ccc(C(F)(F)F)cc1] | 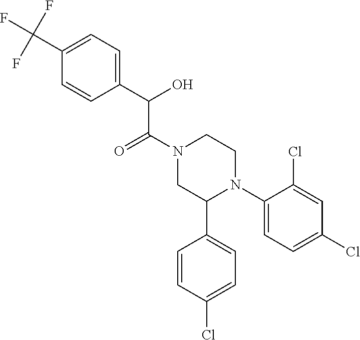 [SMILES: O=C(C(O)c1ccc(C(F)(F)F)cc1)N1CCN(c2ccc(Cl)cc2Cl)C(c2ccc(Cl)cc2)C1] |
| 631 | 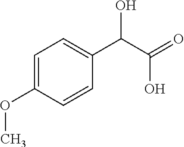 | 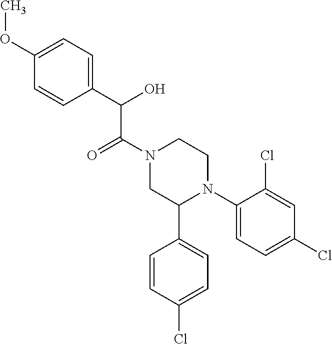 [SMILES: COc1ccc(C(O)C(=O)N2CCN(c3ccc(Cl)cc3Cl)C(c3ccc(Cl)cc3)C2)cc1] |
| 632 | 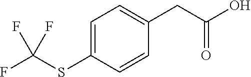 [SMILES: O=C(O)Cc1ccc(SC(F)(F)F)cc1] | 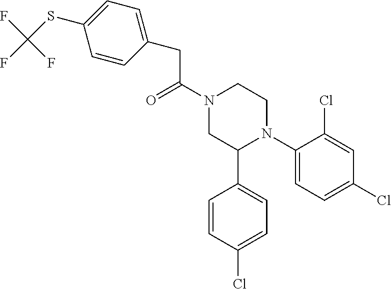 [SMILES: O=C(Cc1ccc(SC(F)(F)F)cc1)N1CCN(c2ccc(Cl)cc2Cl)C(c2ccc(Cl)cc2)C1] |
| 633 | 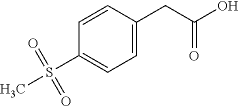 | 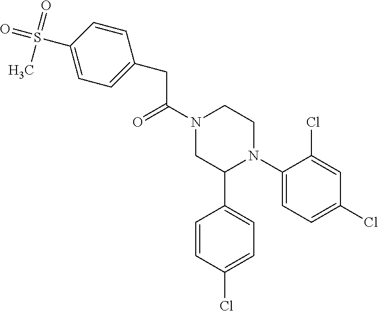 [SMILES: CS(=O)(=O)c1ccc(CC(=O)N2CCN(c3ccc(Cl)cc3Cl)C(c3ccc(Cl)cc3)C2)cc1] |
| 634 | 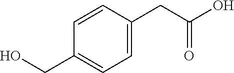 [SMILES: O=C(O)Cc1ccc(CO)cc1] | 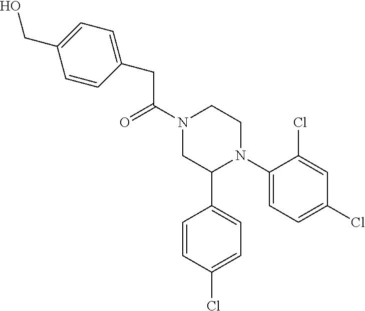 [SMILES: O=C(Cc1ccc(CO)cc1)N1CCN(c2ccc(Cl)cc2Cl)C(c2ccc(Cl)cc2)C1] |
| 635 | 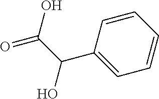 [SMILES: O=C(O)C(O)c1ccccc1] | 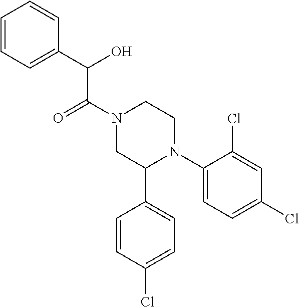 [SMILES: O=C(C(O)c1ccccc1)N1CCN(c2ccc(Cl)cc2Cl)C(c2ccc(Cl)cc2)C1] |
| 636 | 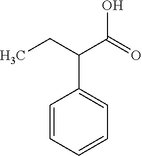 | 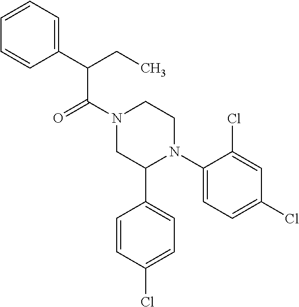 [SMILES: CCC(C(=O)N1CCN(c2ccc(Cl)cc2Cl)C(c2ccc(Cl)cc2)C1)c1ccccc1] |
| 637 | 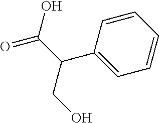 [SMILES: O=C(O)C(CO)c1ccccc1] | 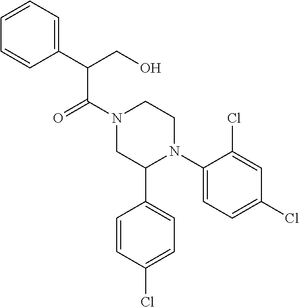 [SMILES: O=C(C(CO)c1ccccc1)N1CCN(c2ccc(Cl)cc2Cl)C(c2ccc(Cl)cc2)C1] |
| 638 | 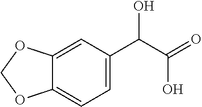 [SMILES: O=C(O)C(O)c1ccc2c(c1)OCO2] | 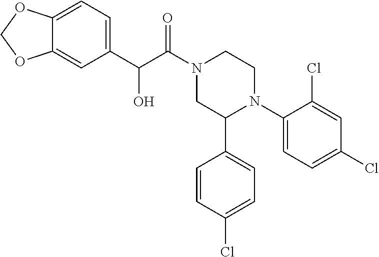 [SMILES: O=C(C(O)c1ccc2c(c1)OCO2)N1CCN(c2ccc(Cl)cc2Cl)C(c2ccc(Cl)cc2)C1] |
| 639 | 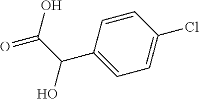 [SMILES: O=C(O)C(O)c1ccc(Cl)cc1] | 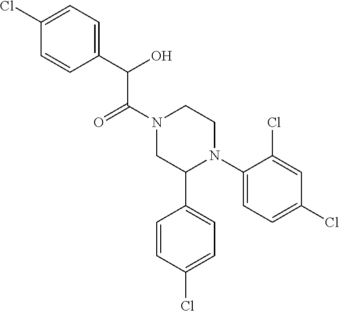 [SMILES: O=C(C(O)c1ccc(Cl)cc1)N1CCN(c2ccc(Cl)cc2Cl)C(c2ccc(Cl)cc2)C1] |
| 640 | 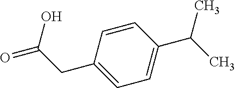 | 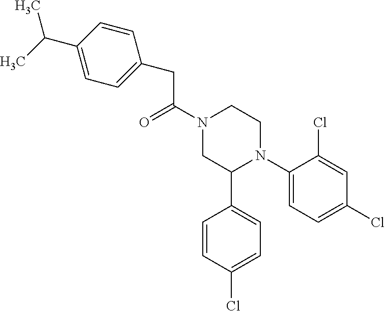 [SMILES: CC(C)c1ccc(CC(=O)N2CCN(c3ccc(Cl)cc3Cl)C(c3ccc(Cl)cc3)C2)cc1] |
| 641 | 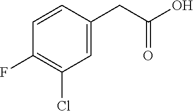 [SMILES: O=C(O)Cc1ccc(F)c(Cl)c1] | 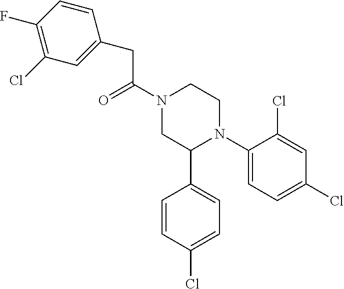 [SMILES: O=C(Cc1ccc(F)c(Cl)c1)N1CCN(c2ccc(Cl)cc2Cl)C(c2ccc(Cl)cc2)C1] |
| 642 | 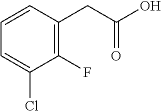 [SMILES: O=C(O)Cc1cccc(Cl)c1F] | 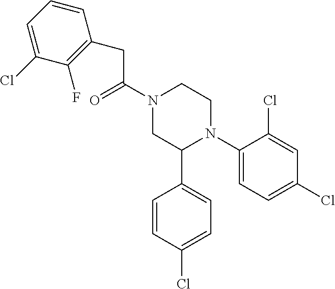 [SMILES: O=C(Cc1cccc(Cl)c1F)N1CCN(c2ccc(Cl)cc2Cl)C(c2ccc(Cl)cc2)C1] |
| 643 | 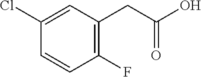 [SMILES: O=C(O)Cc1cc(Cl)ccc1F] | 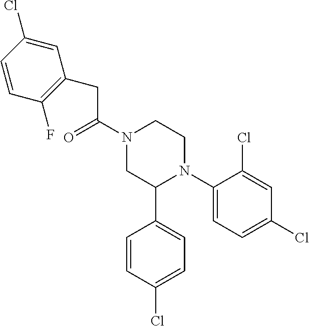 [SMILES: O=C(Cc1cc(Cl)ccc1F)N1CCN(c2ccc(Cl)cc2Cl)C(c2ccc(Cl)cc2)C1] |
| 644 | 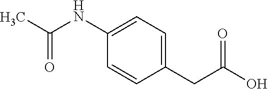 | 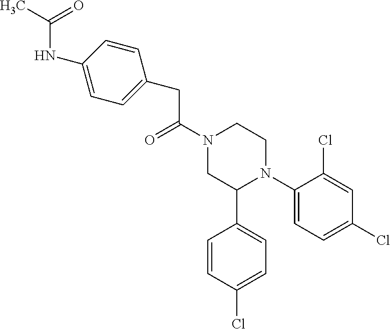 [SMILES: CC(=O)Nc1ccc(CC(=O)N2CCN(c3ccc(Cl)cc3Cl)C(c3ccc(Cl)cc3)C2)cc1] |
| 645 | 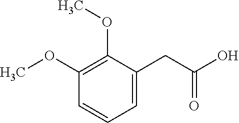 | 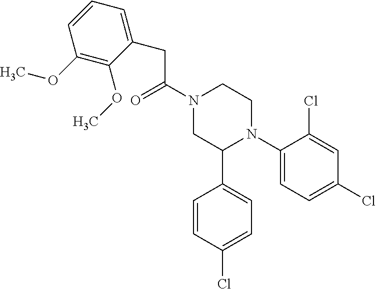 [SMILES: COc1cccc(CC(=O)N2CCN(c3ccc(Cl)cc3Cl)C(c3ccc(Cl)cc3)C2)c1OC] |
| 646 | 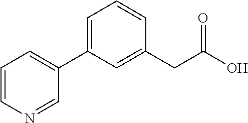 [SMILES: O=C(O)Cc1cccc(-c2cccnc2)c1] | 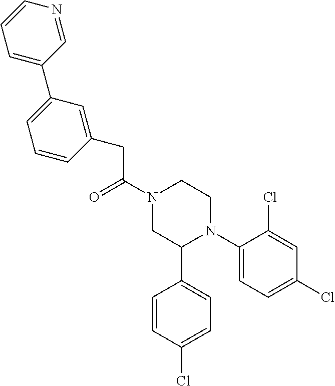 [SMILES: O=C(Cc1cccc(-c2cccnc2)c1)N1CCN(c2ccc(Cl)cc2Cl)C(c2ccc(Cl)cc2)C1] |
| 647 | 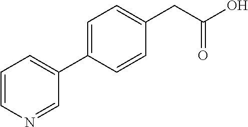 [SMILES: O=C(O)Cc1ccc(-c2cccnc2)cc1] | 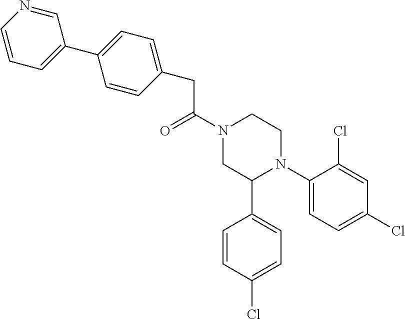 [SMILES: O=C(Cc1ccc(-c2cccnc2)cc1)N1CCN(c2ccc(Cl)cc2Cl)C(c2ccc(Cl)cc2)C1] |
| 648 | 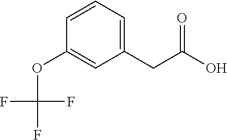 [SMILES: O=C(O)Cc1cccc(OC(F)(F)F)c1] | 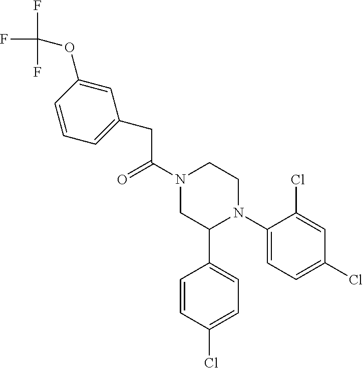 [SMILES: O=C(Cc1cccc(OC(F)(F)F)c1)N1CCN(c2ccc(Cl)cc2Cl)C(c2ccc(Cl)cc2)C1] |
| 649 | 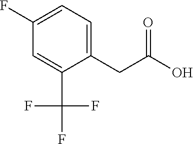 [SMILES: O=C(O)Cc1ccc(F)cc1C(F)(F)F] | 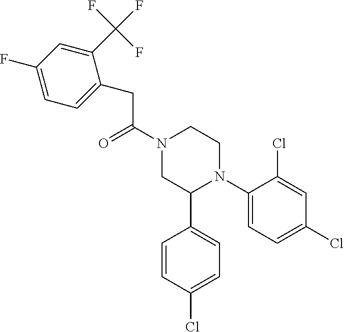 [SMILES: O=C(Cc1ccc(F)cc1C(F)(F)F)N1CCN(c2ccc(Cl)cc2Cl)C(c2ccc(Cl)cc2)C1] |
| 650 | 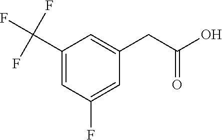 [SMILES: O=C(O)Cc1cc(F)cc(C(F)(F)F)c1] | 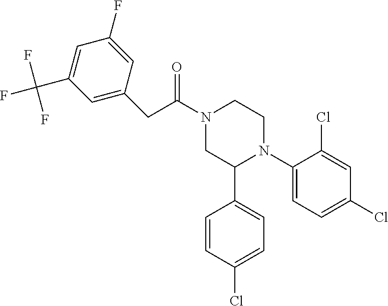 [SMILES: O=C(Cc1cc(F)cc(C(F)(F)F)c1)N1CCN(c2ccc(Cl)cc2Cl)C(c2ccc(Cl)cc2)C1] |
| 651 | 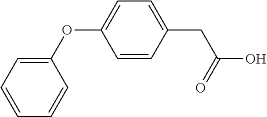 [SMILES: O=C(O)Cc1ccc(Oc2ccccc2)cc1] | 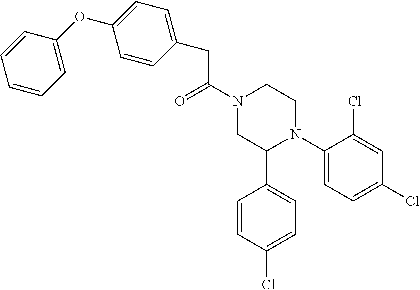 [SMILES: O=C(Cc1ccc(Oc2ccccc2)cc1)N1CCN(c2ccc(Cl)cc2Cl)C(c2ccc(Cl)cc2)C1] |
| 652 | 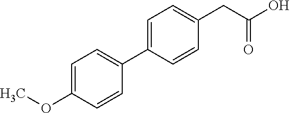 [SMILES: COc1ccc(-c2ccc(CC(=O)O)cc2)cc1] | 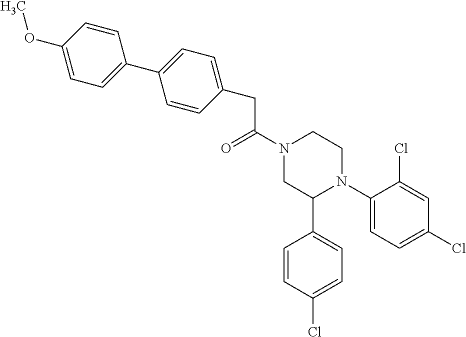 [SMILES: COc1ccc(-c2ccc(CC(=O)N3CCN(c4ccc(Cl)cc4Cl)C(c4ccc(Cl)cc4)C3)cc2)cc1] |
| 653 | 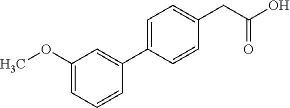 [SMILES: COc1cccc(-c2ccc(CC(=O)O)cc2)c1] | 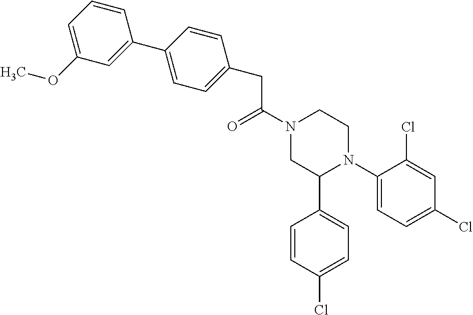 [SMILES: COc1cccc(-c2ccc(CC(=O)N3CCN(c4ccc(Cl)cc4Cl)C(c4ccc(Cl)cc4)C3)cc2)c1] |
| 654 | 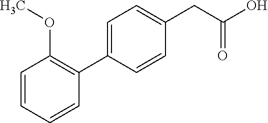 [SMILES: COc1ccccc1-c1ccc(CC(=O)O)cc1] | 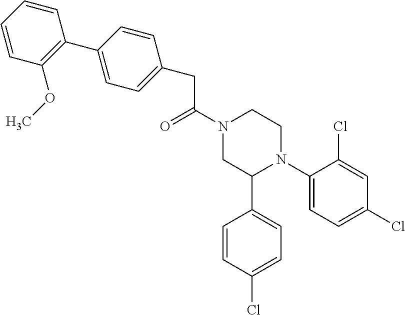 [SMILES: COc1ccccc1-c1ccc(CC(=O)N2CCN(c3ccc(Cl)cc3Cl)C(c3ccc(Cl)cc3)C2)cc1] |
| 655 | 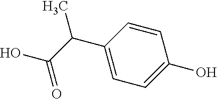 | 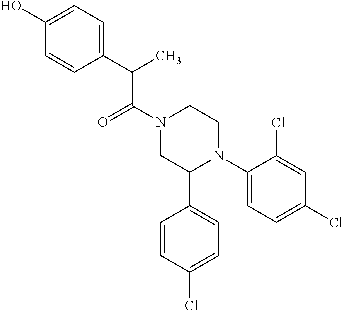 [SMILES: CC(C(=O)N1CCN(c2ccc(Cl)cc2Cl)C(c2ccc(Cl)cc2)C1)c1ccc(O)cc1] |
| 656 | 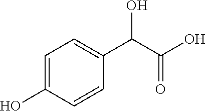 [SMILES: O=C(O)C(O)c1ccc(O)cc1] | 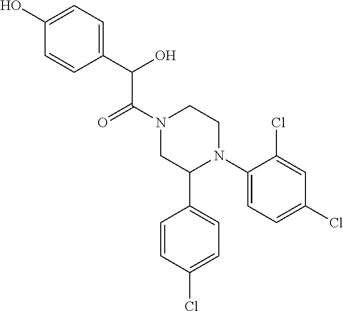 [SMILES: O=C(C(O)c1ccc(O)cc1)N1CCN(c2ccc(Cl)cc2Cl)C(c2ccc(Cl)cc2)C1] |
| 657 | 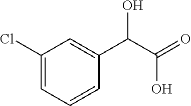 [SMILES: O=C(O)C(O)c1cccc(Cl)c1] | 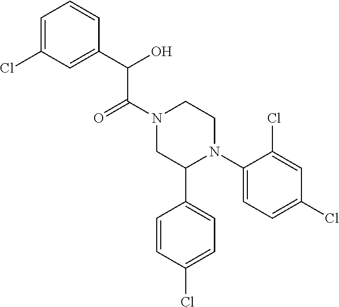 [SMILES: O=C(C(O)c1cccc(Cl)c1)N1CCN(c2ccc(Cl)cc2Cl)C(c2ccc(Cl)cc2)C1] |
| 658 | 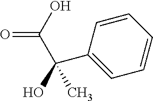 | 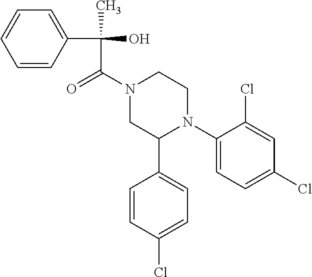 [SMILES: C[C@](O)(C(=O)N1CCN(c2ccc(Cl)cc2Cl)C(c2ccc(Cl)cc2)C1)c1ccccc1] |
| 659 | 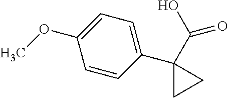 | 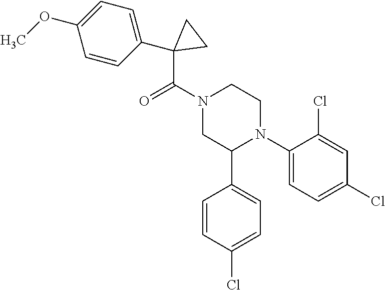 [SMILES: COc1ccc(C2(C(=O)N3CCN(c4ccc(Cl)cc4Cl)C(c4ccc(Cl)cc4)C3)CC2)cc1] |
| 660 | 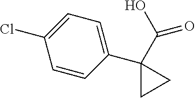 [SMILES: O=C(O)C1(c2ccc(Cl)cc2)CC1] | 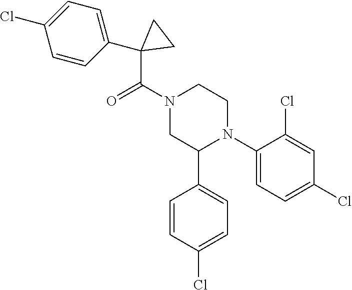 [SMILES: O=C(N1CCN(c2ccc(Cl)cc2Cl)C(c2ccc(Cl)cc2)C1)C1(c2ccc(Cl)cc2)CC1] |
| 661 | 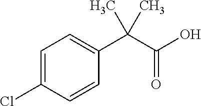 | 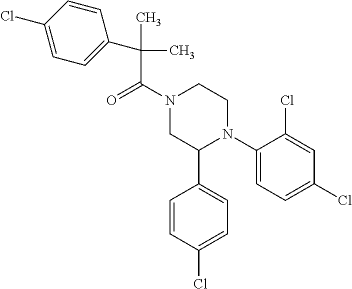 [SMILES: CC(C)(C(=O)N1CCN(c2ccc(Cl)cc2Cl)C(c2ccc(Cl)cc2)C1)c1ccc(Cl)cc1] |
| 662 | 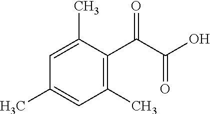 | 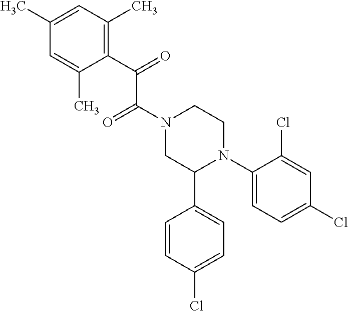 [SMILES: Cc1cc(C)c(C(=O)C(=O)N2CCN(c3ccc(Cl)cc3Cl)C(c3ccc(Cl)cc3)C2)c(C)c1] |
| 663 | 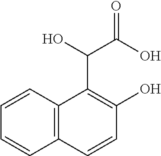 [SMILES: O=C(O)C(O)c1c(O)ccc2ccccc12] | 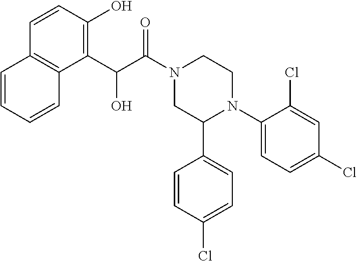 [SMILES: O=C(C(O)c1c(O)ccc2ccccc12)N1CCN(c2ccc(Cl)cc2Cl)C(c2ccc(Cl)cc2)C1] |
| 664 | 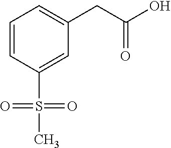 | 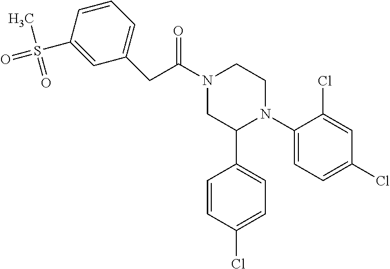 [SMILES: CS(=O)(=O)c1cccc(CC(=O)N2CCN(c3ccc(Cl)cc3Cl)C(c3ccc(Cl)cc3)C2)c1] |
| 665 | 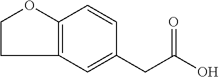 [SMILES: O=C(O)Cc1ccc2c(c1)CCO2] | 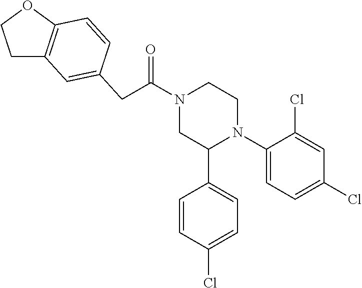 [SMILES: O=C(Cc1ccc2c(c1)CCO2)N1CCN(c2ccc(Cl)cc2Cl)C(c2ccc(Cl)cc2)C1] |
| 666 | 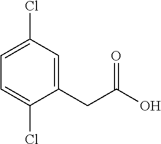 [SMILES: O=C(O)Cc1cc(Cl)ccc1Cl] | 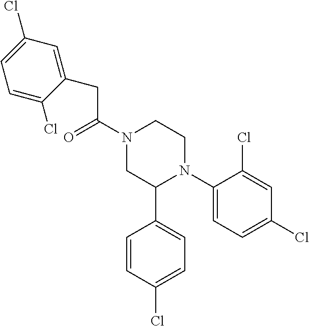 [SMILES: O=C(Cc1cc(Cl)ccc1Cl)N1CCN(c2ccc(Cl)cc2Cl)C(c2ccc(Cl)cc2)C1] |
| 667 | 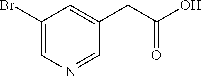 [SMILES: O=C(O)Cc1cncc(Br)c1] | 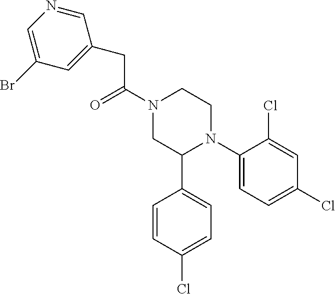 [SMILES: O=C(Cc1cncc(Br)c1)N1CCN(c2ccc(Cl)cc2Cl)C(c2ccc(Cl)cc2)C1] |
| 668 | 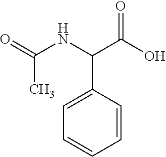 | 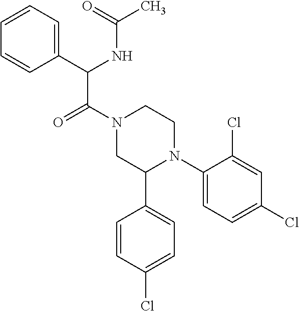 [SMILES: CC(=O)NC(C(=O)N1CCN(c2ccc(Cl)cc2Cl)C(c2ccc(Cl)cc2)C1)c1ccccc1] |
| 669 | 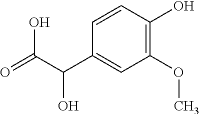 | 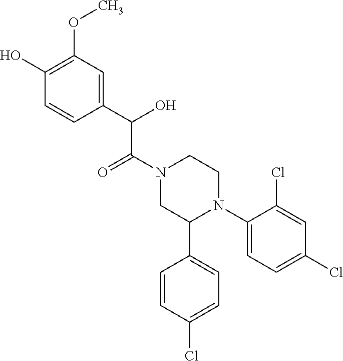 [SMILES: COc1cc(C(O)C(=O)N2CCN(c3ccc(Cl)cc3Cl)C(c3ccc(Cl)cc3)C2)ccc1O] |
| 670 | 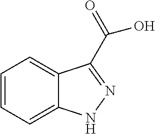 [SMILES: O=C(O)c1n[nH]c2ccccc12] | 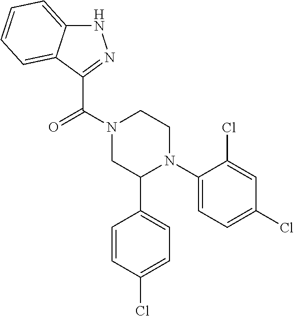 [SMILES: O=C(c1n[nH]c2ccccc12)N1CCN(c2ccc(Cl)cc2Cl)C(c2ccc(Cl)cc2)C1] |
| 671 | 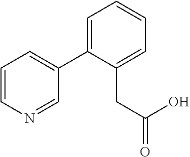 [SMILES: O=C(O)Cc1ccccc1-c1cccnc1] | 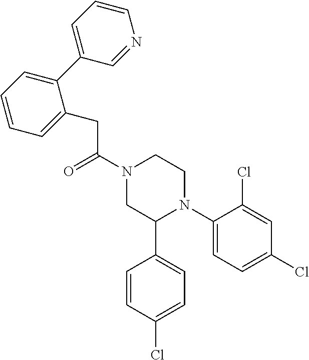 [SMILES: O=C(Cc1ccccc1-c1cccnc1)N1CCN(c2ccc(Cl)cc2Cl)C(c2ccc(Cl)cc2)C1] |
| 672 | 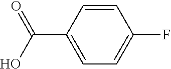 | 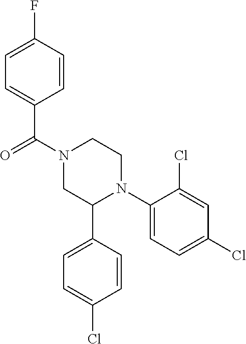 [SMILES: O=C(c1ccc(F)cc1)N1CCN(c2ccc(Cl)cc2Cl)C(c2ccc(Cl)cc2)C1] |
| 673 | 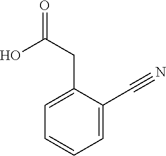 [SMILES: N#Cc1ccccc1CC(=O)O] | 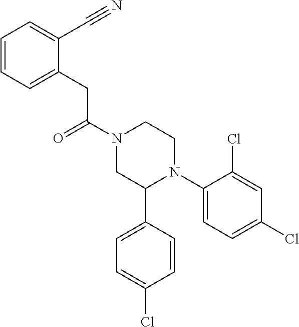 [SMILES: N#Cc1ccccc1CC(=O)N1CCN(c2ccc(Cl)cc2Cl)C(c2ccc(Cl)cc2)C1] |
| 674 | 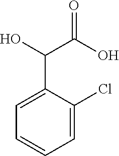 [SMILES: O=C(O)C(O)c1ccccc1Cl] | 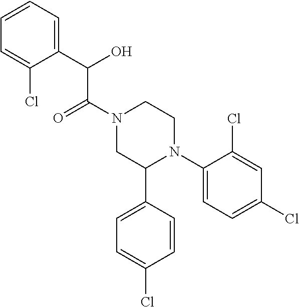 [SMILES: O=C(C(O)c1ccccc1Cl)N1CCN(c2ccc(Cl)cc2Cl)C(c2ccc(Cl)cc2)C1] |
| 675 | 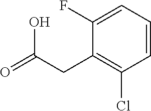 [SMILES: O=C(O)Cc1c(F)cccc1Cl] | 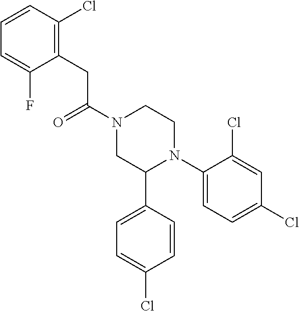 [SMILES: O=C(Cc1c(F)cccc1Cl)N1CCN(c2ccc(Cl)cc2Cl)C(c2ccc(Cl)cc2)C1] |
| 676 | 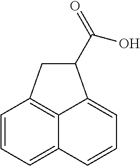 [SMILES: O=C(O)C1Cc2cccc3cccc1c23] | 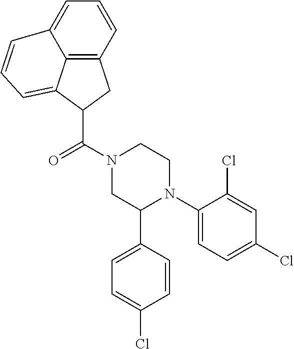 [SMILES: O=C(C1Cc2cccc3cccc1c23)N1CCN(c2ccc(Cl)cc2Cl)C(c2ccc(Cl)cc2)C1] |
| 677 | 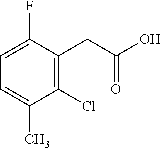 | 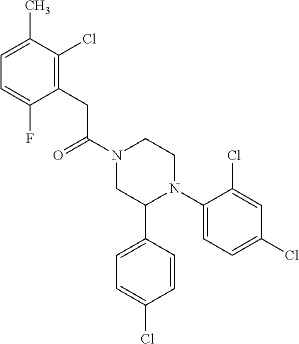 [SMILES: Cc1ccc(F)c(CC(=O)N2CCN(c3ccc(Cl)cc3Cl)C(c3ccc(Cl)cc3)C2)c1Cl] |
| 678 | 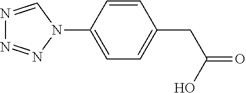 [SMILES: O=C(O)Cc1ccc(-n2cnnn2)cc1] | 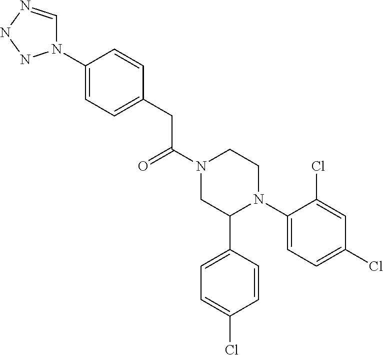 [SMILES: O=C(Cc1ccc(N2C=NNN2)cc1)N1CCN(c2ccc(Cl)cc2Cl)C(c2ccc(Cl)cc2)C1] |
| 679 | 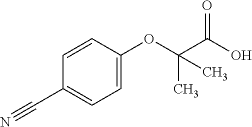 | 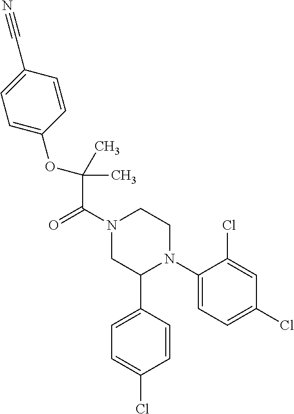 [SMILES: CC(C)(Oc1ccc(C#N)cc1)C(=O)N1CCN(c2ccc(Cl)cc2Cl)C(c2ccc(Cl)cc2)C1] |
| 680 | 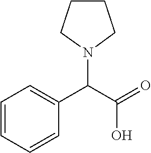 [SMILES: O=C(O)C(c1ccccc1)N1CCCC1] | 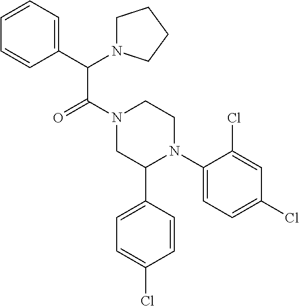 [SMILES: O=C(C(c1ccccc1)N1CCCC1)N1CCN(c2ccc(Cl)cc2Cl)C(c2ccc(Cl)cc2)C1] |
| 681 | 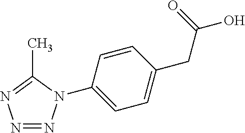 [SMILES: Cc1nnnn1-c1ccc(CC(=O)O)cc1] | 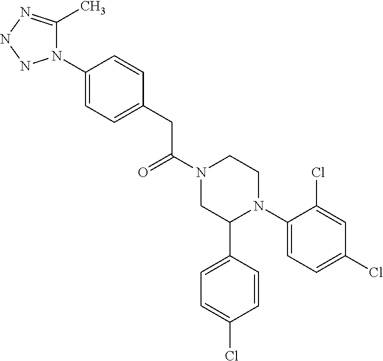 [SMILES: CC1=NNNN1c1ccc(CC(=O)N2CCN(c3ccc(Cl)cc3Cl)C(c3ccc(Cl)cc3)C2)cc1] |
| 682 | 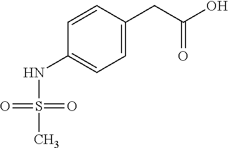 | 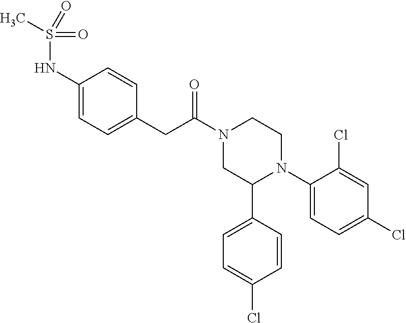 [SMILES: CS(=O)(=O)Nc1ccc(CC(=O)N2CCN(c3ccc(Cl)cc3Cl)C(c3ccc(Cl)cc3)C2)cc1] |
| 683 | 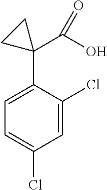 [SMILES: O=C(O)C1(c2ccc(Cl)cc2Cl)CC1] | 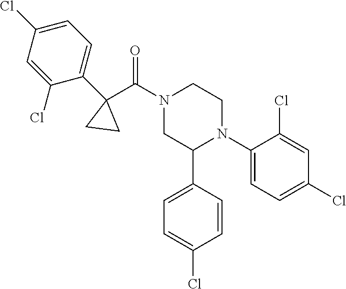 [SMILES: O=C(N1CCN(c2ccc(Cl)cc2Cl)C(c2ccc(Cl)cc2)C1)C1(c2ccc(Cl)cc2Cl)CC1] |
| 684 | 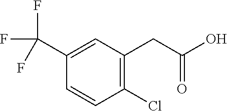 [SMILES: O=C(O)Cc1cc(C(F)(F)F)ccc1Cl] | 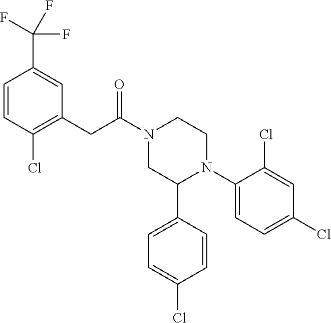 [SMILES: O=C(Cc1cc(C(F)(F)F)ccc1Cl)N1CCN(c2ccc(Cl)cc2Cl)C(c2ccc(Cl)cc2)C1] |
| 685 | 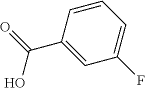 | 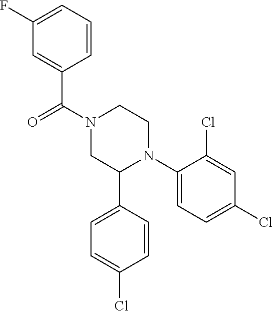 [SMILES: O=C(c1cccc(F)c1)N1CCN(c2ccc(Cl)cc2Cl)C(c2ccc(Cl)cc2)C1] |

### Preparation of Examples 686-766

Examples 686-766 were prepared by the reaction of piperazine Example 304 with the carboxylic acids shown in Table XXI, using the procedure of Scheme 5. Examples 735, 737, 739, 741, 743, 745, 747, 749, 751, 753, 755, 757, 759, 761, 763, and 765 prepared from the corresponding Boc carbamate by hydrolysis in 95% MeOH/5% (95% TFA/5% H₂O) solution in pre-weighed vials with shaking for 3 h. The solvent was removed in vacuo using a SpeedVac. Methanol and 2N HCl/ether was added and the samples shaken for 3 h. The solvent was removed in vacuo on a SpeedVac and the products were isolated as the HCl salt.

TABLE XXI

| Example # | Carboxylic Acid | Example Structure |
|---|---|---|
| 686 | 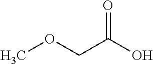 | 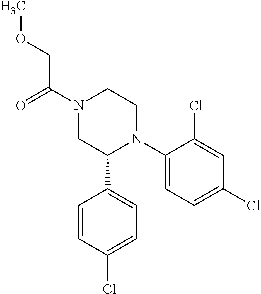 [SMILES: COCC(=O)N1CCN(c2ccc(Cl)cc2Cl)[C@H](c2ccc(Cl)cc2)C1] |
| 687 | 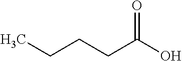 | 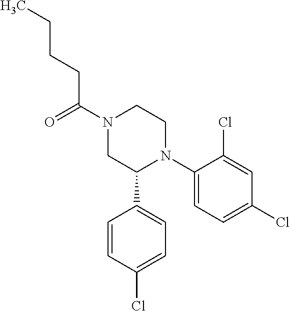 [SMILES: CCCCC(=O)N1CCN(c2ccc(Cl)cc2Cl)[C@H](c2ccc(Cl)cc2)C1] |
| 688 | 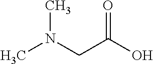 | 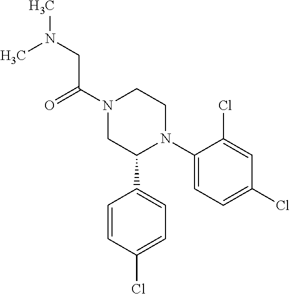 [SMILES: CN(C)CC(=O)N1CCN(c2ccc(Cl)cc2Cl)[C@H](c2ccc(Cl)cc2)C1] |
| 689 | 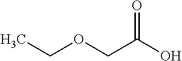 | 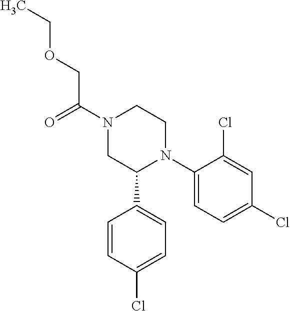 [SMILES: CCOCC(=O)N1CCN(c2ccc(Cl)cc2Cl)[C@H](c2ccc(Cl)cc2)C1] |
| 690 | 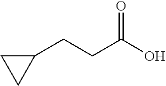 | 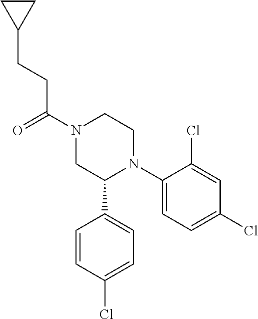 [SMILES: O=C(CCC1CC1)N1CCN(c2ccc(Cl)cc2Cl)[C@H](c2ccc(Cl)cc2)C1] |
| 691 | 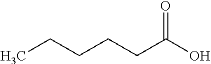 | 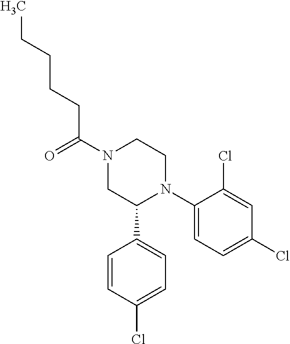 [SMILES: CCCCCC(=O)N1CCN(c2ccc(Cl)cc2Cl)[C@H](c2ccc(Cl)cc2)C1] |
| 692 | 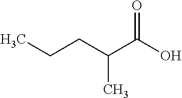 | 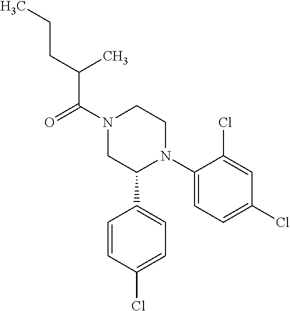 [SMILES: CCCC(C)C(=O)N1CCN(c2ccc(Cl)cc2Cl)[C@H](c2ccc(Cl)cc2)C1] |
| 693 | 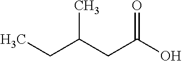 | 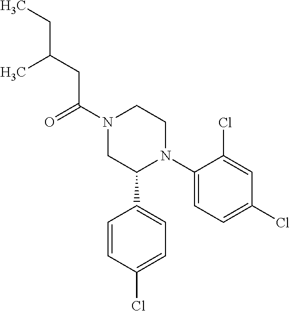 [SMILES: CCC(C)CC(=O)N1CCN(c2ccc(Cl)cc2Cl)[C@H](c2ccc(Cl)cc2)C1] |
| 694 | 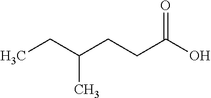 | 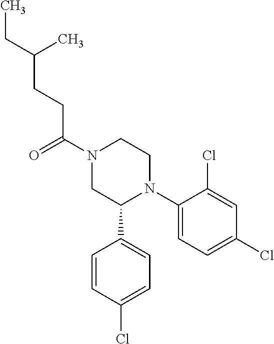 [SMILES: CCC(C)CCC(=O)N1CCN(c2ccc(Cl)cc2Cl)[C@H](c2ccc(Cl)cc2)C1] |
| 695 | 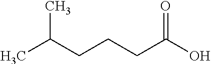 | 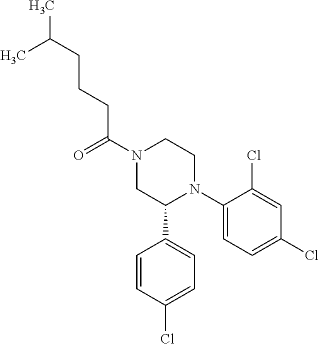 [SMILES: CC(C)CCCC(=O)N1CCN(c2ccc(Cl)cc2Cl)[C@H](c2ccc(Cl)cc2)C1] |
| 696 | 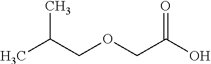 | 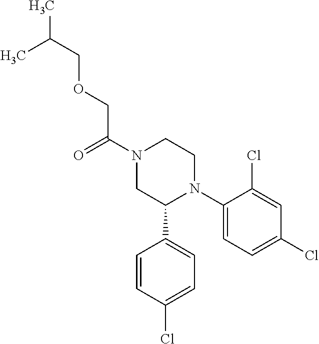 [SMILES: CC(C)COCC(=O)N1CCN(c2ccc(Cl)cc2Cl)[C@H](c2ccc(Cl)cc2)C1] |
| 697 | 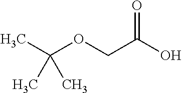 | 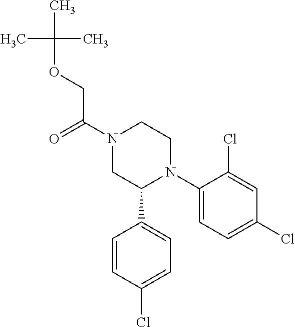 [SMILES: CC(C)(C)OCC(=O)N1CCN(c2ccc(Cl)cc2Cl)[C@H](c2ccc(Cl)cc2)C1] |
| 698 | 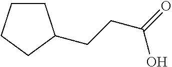 | 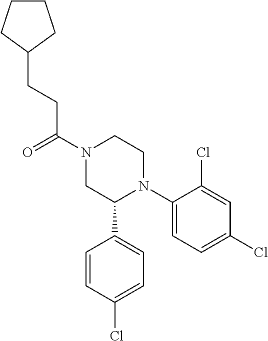 [SMILES: O=C(CCC1CCCC1)N1CCN(c2ccc(Cl)cc2Cl)[C@H](c2ccc(Cl)cc2)C1] |
| 699 | 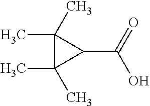 | 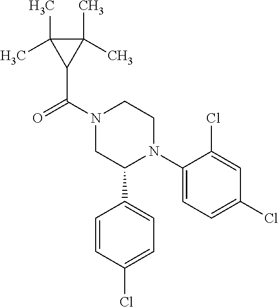 [SMILES: CC1(C)C(C(=O)N2CCN(c3ccc(Cl)cc3Cl)[C@H](c3ccc(Cl)cc3)C2)C1(C)C] |
| 700 | 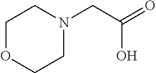 | 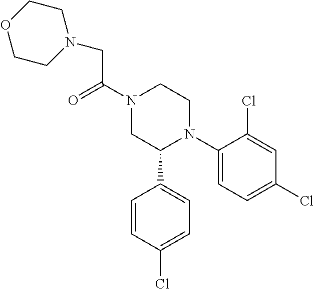 [SMILES: O=C(CN1CCOCC1)N1CCN(c2ccc(Cl)cc2Cl)[C@H](c2ccc(Cl)cc2)C1] |
| 701 | 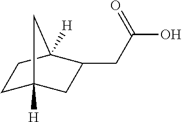 [SMILES: O=C(O)CC1C[C@@H]2CC[C@H]1C2] | 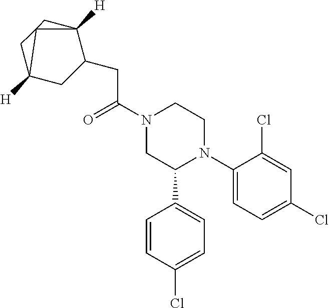 [SMILES: O=C(CC1C[C@@H]2CC23C[C@H]13)N1CCN(c2ccc(Cl)cc2Cl)[C@H](c2ccc(Cl)cc2)C1] |
| 702 | 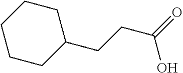 [SMILES: O=C(O)CCC1CCCCC1] | 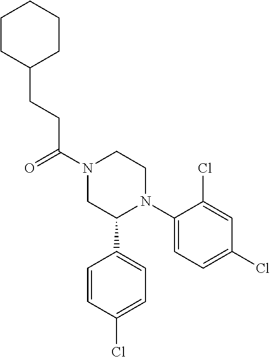 [SMILES: O=C(CCC1CCCCC1)N1CCN(c2ccc(Cl)cc2Cl)[C@H](c2ccc(Cl)cc2)C1] |
| 703 | 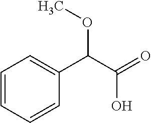 | 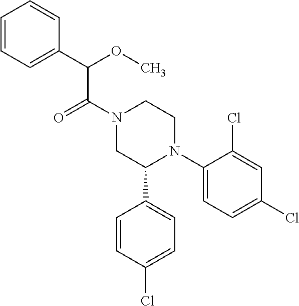 [SMILES: COC(C(=O)N1CCN(c2ccc(Cl)cc2Cl)[C@H](c2ccc(Cl)cc2)C1)c1ccccc1] |
| 704 | 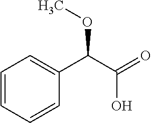 | 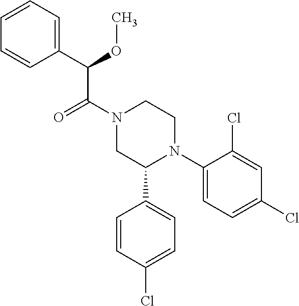 [SMILES: CO[C@@H](C(=O)N1CCN(c2ccc(Cl)cc2Cl)[C@H](c2ccc(Cl)cc2)C1)c1ccccc1] |
| 705 | 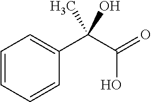 | 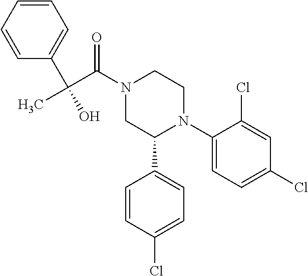 [SMILES: C[C@](O)(C(=O)N1CCN(c2ccc(Cl)cc2Cl)[C@H](c2ccc(Cl)cc2)C1)c1ccccc1] |
| 706 | 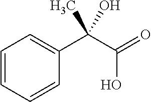 | 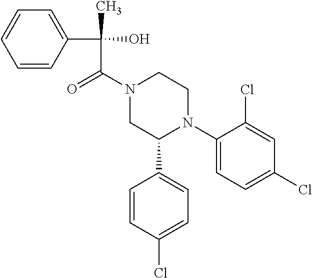 [SMILES: C[C@@](O)(C(=O)N1CCN(c2ccc(Cl)cc2Cl)[C@H](c2ccc(Cl)cc2)C1)c1ccccc1] |
| 707 | 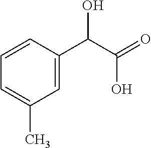 | 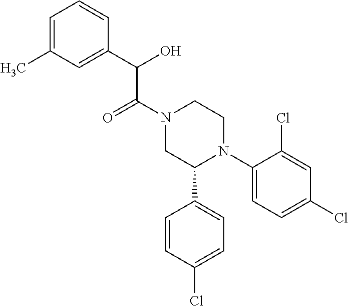 [SMILES: Cc1cccc(C(O)C(=O)N2CCN(c3ccc(Cl)cc3Cl)[C@H](c3ccc(Cl)cc3)C2)c1] |
| 708 | 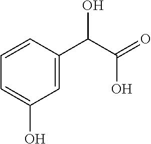 [SMILES: O=C(O)C(O)c1cccc(O)c1] | 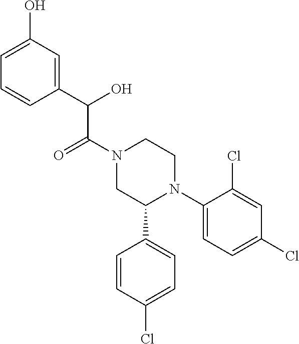 [SMILES: O=C(C(O)c1cccc(O)c1)N1CCN(c2ccc(Cl)cc2Cl)[C@H](c2ccc(Cl)cc2)C1] |
| 709 | 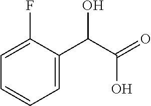 [SMILES: O=C(O)C(O)c1ccccc1F] | 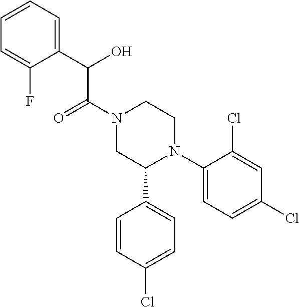 [SMILES: O=C(C(O)c1ccccc1F)N1CCN(c2ccc(Cl)cc2Cl)[C@H](c2ccc(Cl)cc2)C1] |
| 710 | 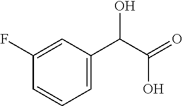 [SMILES: O=C(O)C(O)c1cccc(F)c1] | 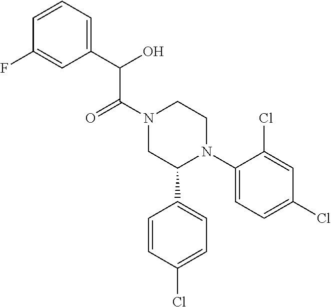 [SMILES: O=C(C(O)c1cccc(F)c1)N1CCN(c2ccc(Cl)cc2Cl)[C@H](c2ccc(Cl)cc2)C1] |
| 711 | 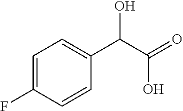 [SMILES: O=C(O)C(O)c1ccc(F)cc1] | 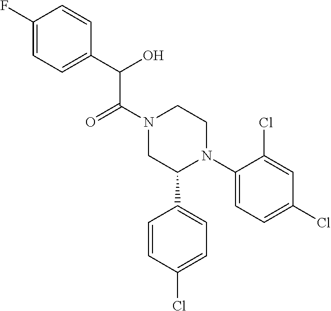 [SMILES: O=C(C(O)c1ccc(F)cc1)N1CCN(c2ccc(Cl)cc2Cl)[C@H](c2ccc(Cl)cc2)C1] |
| 712 | 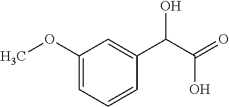 | 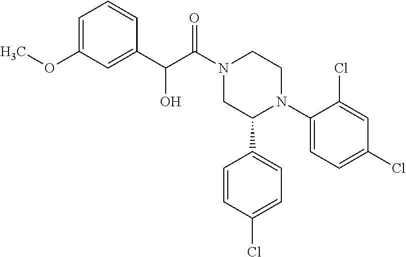 [SMILES: COc1cccc(C(O)C(=O)N2CCN(c3ccc(Cl)cc3Cl)[C@H](c3ccc(Cl)cc3)C2)c1] |
| 713 | 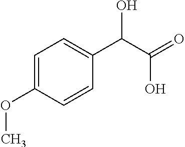 | 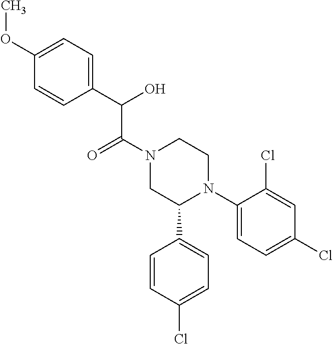 [SMILES: COc1ccc(C(O)C(=O)N2CCN(c3ccc(Cl)cc3Cl)[C@H](c3ccc(Cl)cc3)C2)cc1] |
| 714 | 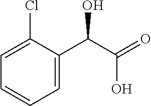 [SMILES: O=C(O)[C@H](O)c1ccccc1Cl] | 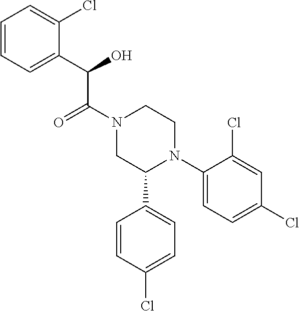 [SMILES: O=C([C@H](O)c1ccccc1Cl)N1CCN(c2ccc(Cl)cc2Cl)[C@H](c2ccc(Cl)cc2)C1] |
| 715 | 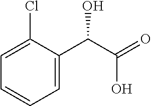 [SMILES: O=C(O)[C@@H](O)c1ccccc1Cl] | 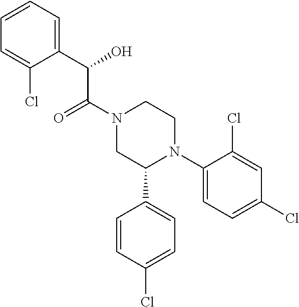 [SMILES: O=C([C@@H](O)c1ccccc1Cl)N1CCN(c2ccc(Cl)cc2Cl)[C@H](c2ccc(Cl)cc2)C1] |
| 716 | 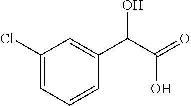 [SMILES: O=C(O)C(O)c1cccc(Cl)c1] | 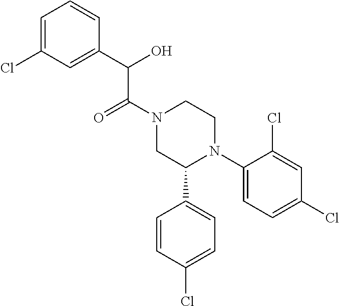 [SMILES: O=C(C(O)c1cccc(Cl)c1)N1CCN(c2ccc(Cl)cc2Cl)[C@H](c2ccc(Cl)cc2)C1] |
| 717 | 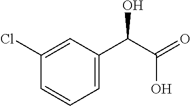 [SMILES: O=C(O)[C@H](O)c1cccc(Cl)c1] | 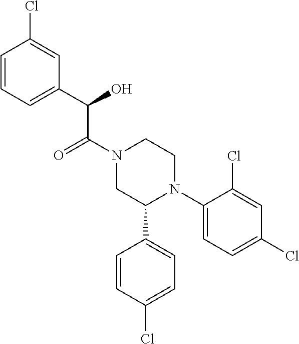 [SMILES: O=C([C@H](O)c1cccc(Cl)c1)N1CCN(c2ccc(Cl)cc2Cl)[C@H](c2ccc(Cl)cc2)C1] |
| 718 | 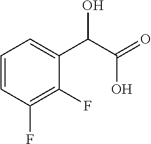 [SMILES: O=C(O)C(O)c1cccc(F)c1F] | 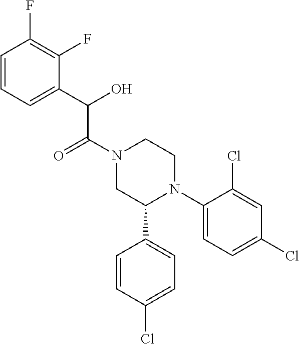 [SMILES: O=C(C(O)c1cccc(F)c1F)N1CCN(c2ccc(Cl)cc2Cl)[C@H](c2ccc(Cl)cc2)C1] |
| 719 | 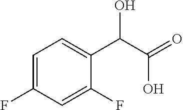 [SMILES: O=C(O)C(O)c1ccc(F)cc1F] | 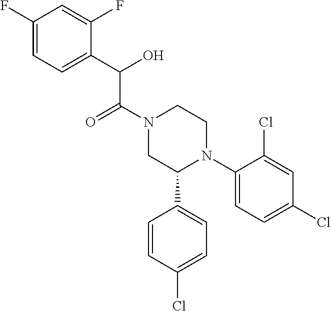 [SMILES: O=C(C(O)c1ccc(F)cc1F)N1CCN(c2ccc(Cl)cc2Cl)[C@H](c2ccc(Cl)cc2)C1] |
| 720 | 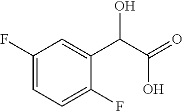 [SMILES: O=C(O)C(O)c1cc(F)ccc1F] | 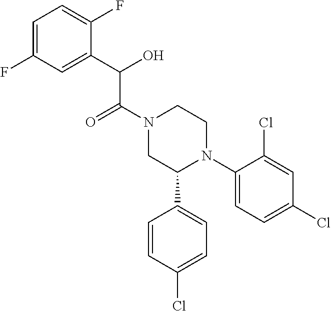 [SMILES: O=C(C(O)c1cc(F)ccc1F)N1CCN(c2ccc(Cl)cc2Cl)[C@H](c2ccc(Cl)cc2)C1] |
| 721 | 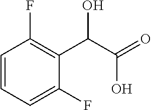 [SMILES: O=C(O)C(O)c1c(F)cccc1F] | 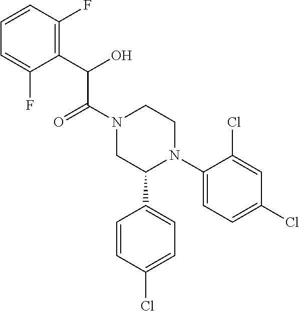 [SMILES: O=C(C(O)c1c(F)cccc1F)N1CCN(c2ccc(Cl)cc2Cl)[C@H](c2ccc(Cl)cc2)C1] |
| 722 | 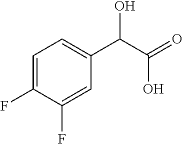 [SMILES: O=C(O)C(O)c1ccc(F)c(F)c1] | 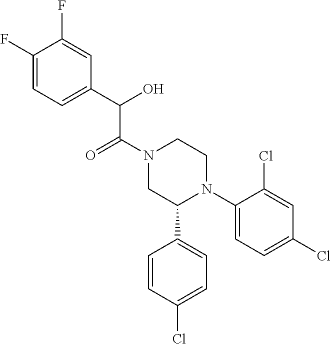 [SMILES: O=C(C(O)c1ccc(F)c(F)c1)N1CCN(c2ccc(Cl)cc2Cl)[C@H](c2ccc(Cl)cc2)C1] |
| 723 | 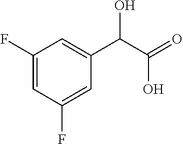 [SMILES: O=C(O)C(O)c1cc(F)cc(F)c1] | 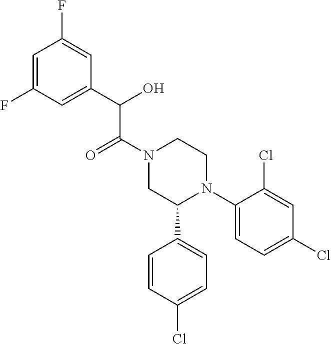 [SMILES: O=C(C(O)c1cc(F)cc(F)c1)N1CCN(c2ccc(Cl)cc2Cl)[C@H](c2ccc(Cl)cc2)C1] |
| 724 | 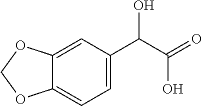 [SMILES: O=C(O)C(O)c1ccc2c(c1)OCO2] | 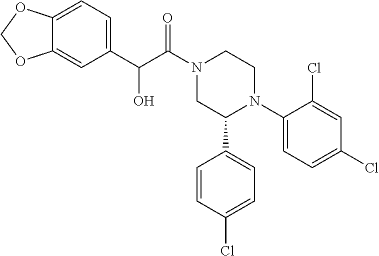 [SMILES: O=C(C(O)c1ccc2c(c1)OCO2)N1CCN(c2ccc(Cl)cc2Cl)[C@H](c2ccc(Cl)cc2)C1] |
| 725 | 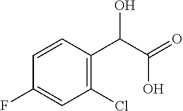 [SMILES: O=C(O)C(O)c1ccc(F)cc1Cl] | 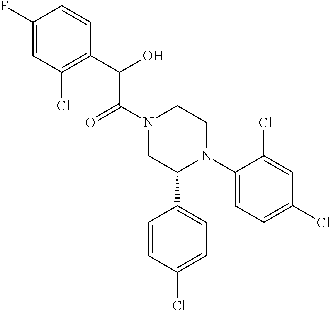 [SMILES: O=C(C(O)c1ccc(F)cc1Cl)N1CCN(c2ccc(Cl)cc2Cl)[C@H](c2ccc(Cl)cc2)C1] |
| 726 | 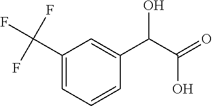 [SMILES: O=C(O)C(O)c1cccc(C(F)(F)F)c1] | 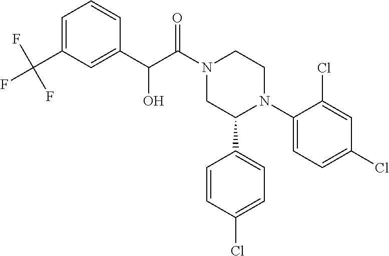 [SMILES: O=C(C(O)c1cccc(C(F)(F)F)c1)N1CCN(c2ccc(Cl)cc2Cl)[C@H](c2ccc(Cl)cc2)C1] |
| 727 | 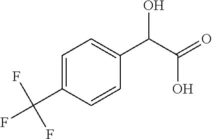 [SMILES: O=C(O)C(O)c1ccc(C(F)(F)F)cc1] | 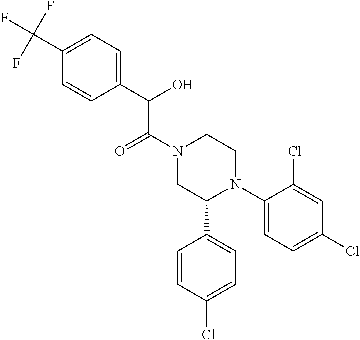 [SMILES: O=C(C(O)c1ccc(C(F)(F)F)cc1)N1CCN(c2ccc(Cl)cc2Cl)[C@H](c2ccc(Cl)cc2)C1] |
| 728 | 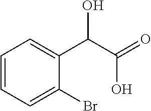 [SMILES: O=C(O)C(O)c1ccccc1Br] | 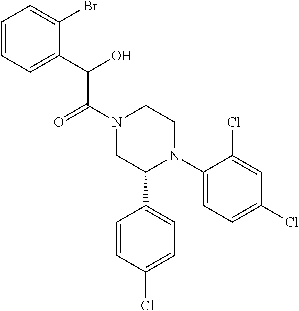 [SMILES: O=C(C(O)c1ccccc1Br)N1CCN(c2ccc(Cl)cc2Cl)[C@H](c2ccc(Cl)cc2)C1] |
| 729 | 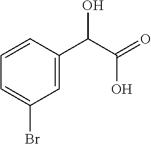 [SMILES: O=C(O)C(O)c1cccc(Br)c1] | 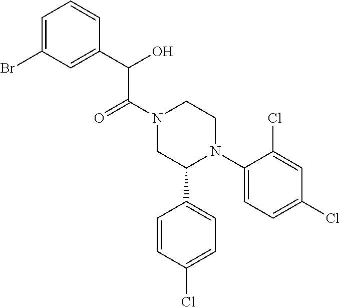 [SMILES: O=C(C(O)c1cccc(Br)c1)N1CCN(c2ccc(Cl)cc2Cl)[C@H](c2ccc(Cl)cc2)C1] |
| 730 | 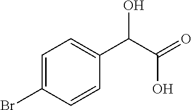 [SMILES: O=C(O)C(O)c1ccc(Br)cc1] | 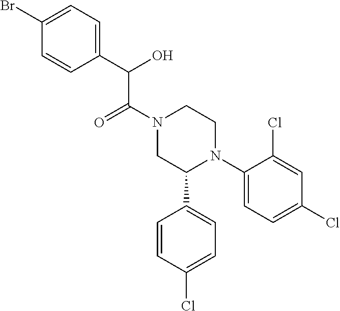 [SMILES: O=C(C(O)c1ccc(Br)cc1)N1CCN(c2ccc(Cl)cc2Cl)[C@H](c2ccc(Cl)cc2)C1] |
| 731 | 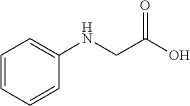 [SMILES: O=C(O)CNc1ccccc1] | 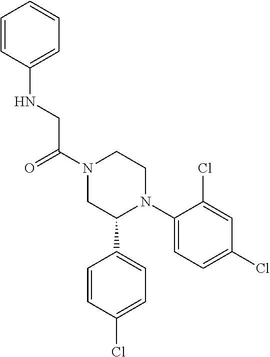 [SMILES: O=C(CNc1ccccc1)N1CCN(c2ccc(Cl)cc2Cl)[C@H](c2ccc(Cl)cc2)C1] |
| 732 | 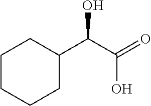 [SMILES: O=C(O)[C@H](O)C1CCCCC1] | 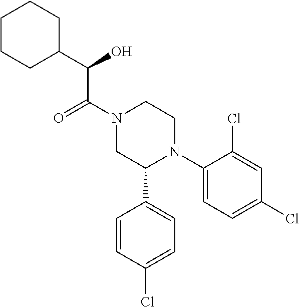 [SMILES: O=C([C@H](O)C1CCCCC1)N1CCN(c2ccc(Cl)cc2Cl)[C@H](c2ccc(Cl)cc2)C1] |
| 733 | 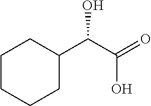 [SMILES: O=C(O)[C@@H](O)C1CCCCC1] | 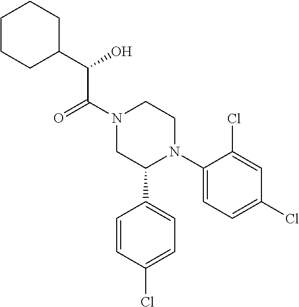 [SMILES: O=C([C@@H](O)C1CCCCC1)N1CCN(c2ccc(Cl)cc2Cl)[C@H](c2ccc(Cl)cc2)C1] |
| 734 | 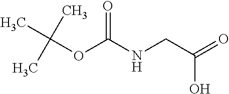 | 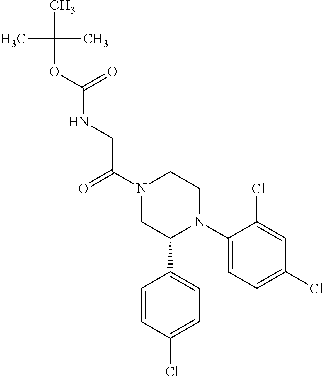 [SMILES: CC(C)(C)OC(=O)NCC(=O)N1CCN(c2ccc(Cl)cc2Cl)[C@H](c2ccc(Cl)cc2)C1] |
| 735 | 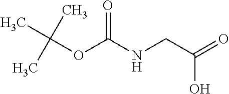 | 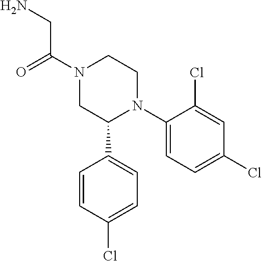 [SMILES: NCC(=O)N1CCN(c2ccc(Cl)cc2Cl)[C@H](c2ccc(Cl)cc2)C1] |
| 736 | 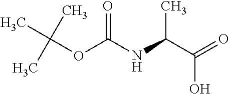 | 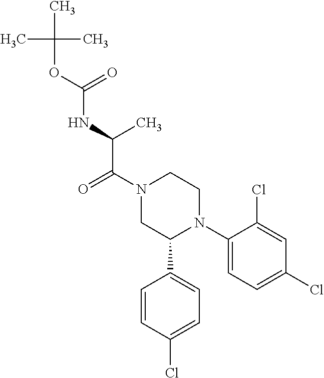 [SMILES: C[C@H](NC(=O)OC(C)(C)C)C(=O)N1CCN(c2ccc(Cl)cc2Cl)[C@H](c2ccc(Cl)cc2)C1] |
| 737 | 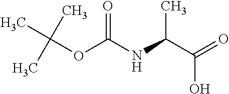 | 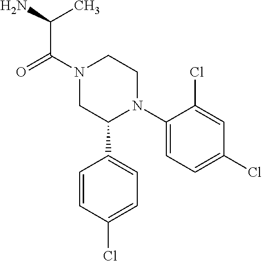 [SMILES: C[C@H](N)C(=O)N1CCN(c2ccc(Cl)cc2Cl)[C@H](c2ccc(Cl)cc2)C1] |
| 738 | 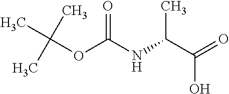 | 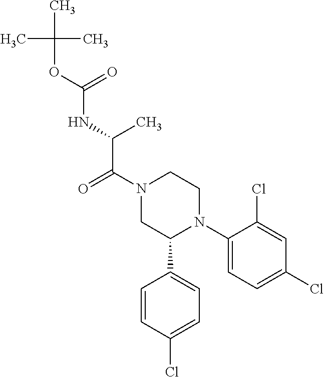 [SMILES: C[C@@H](NC(=O)OC(C)(C)C)C(=O)N1CCN(c2ccc(Cl)cc2Cl)[C@H](c2ccc(Cl)cc2)C1] |
| 739 | 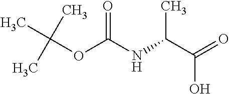 | 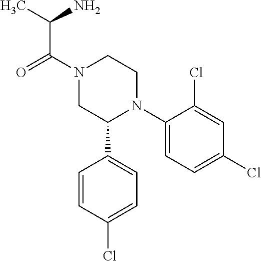 [SMILES: C[C@@H](N)C(=O)N1CCN(c2ccc(Cl)cc2Cl)[C@H](c2ccc(Cl)cc2)C1] |
| 740 | 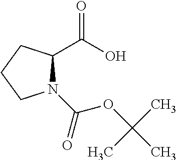 | 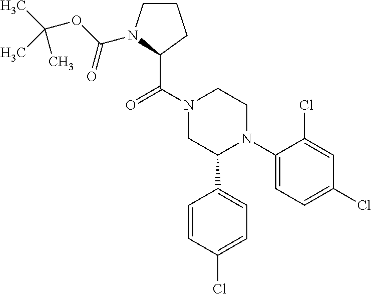 [SMILES: CC(C)(C)OC(=O)N1CCC[C@H]1C(=O)N1CCN(c2ccc(Cl)cc2Cl)[C@H](c2ccc(Cl)cc2)C1] |
| 741 | 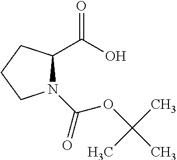 | 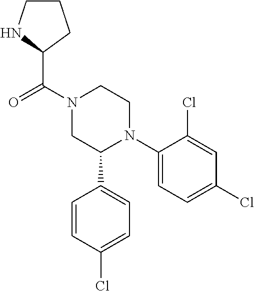 [SMILES: O=C([C@@H]1CCCN1)N1CCN(c2ccc(Cl)cc2Cl)[C@H](c2ccc(Cl)cc2)C1] |
| 742 | 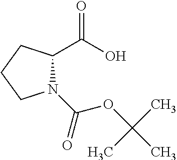 | 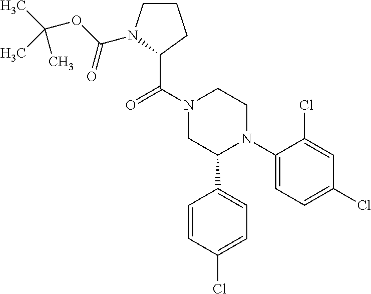 [SMILES: CC(C)(C)OC(=O)N1CCC[C@@H]1C(=O)N1CCN(c2ccc(Cl)cc2Cl)[C@H](c2ccc(Cl)cc2)C1] |
| 743 | 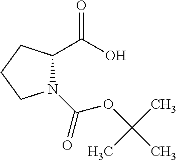 | 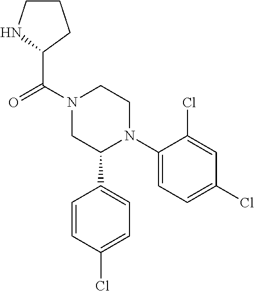 [SMILES: O=C([C@H]1CCCN1)N1CCN(c2ccc(Cl)cc2Cl)[C@H](c2ccc(Cl)cc2)C1] |
| 744 | 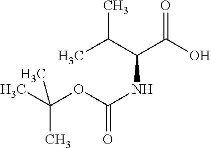 | 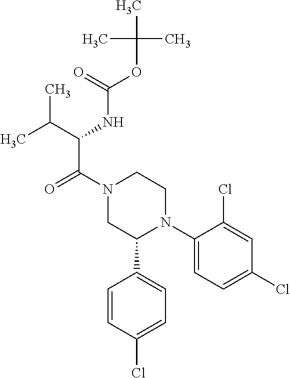 [SMILES: CC(C)[C@H](NC(=O)OC(C)(C)C)C(=O)N1CCN(c2ccc(Cl)cc2Cl)[C@H](c2ccc(Cl)cc2)C1] |
| 745 | 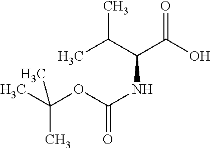 | 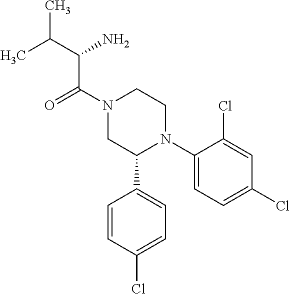 [SMILES: CC(C)[C@H](N)C(=O)N1CCN(c2ccc(Cl)cc2Cl)[C@H](c2ccc(Cl)cc2)C1] |
| 746 | 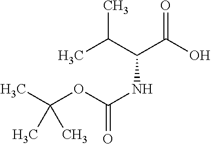 | 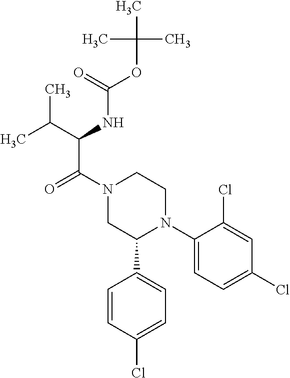 [SMILES: CC(C)[C@@H](NC(=O)OC(C)(C)C)C(=O)N1CCN(c2ccc(Cl)cc2Cl)[C@H](c2ccc(Cl)cc2)C1] |
| 747 | 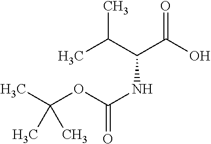 | 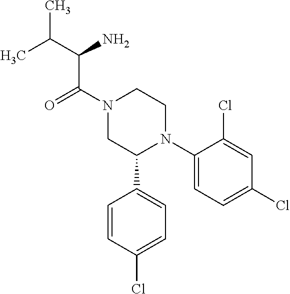 [SMILES: CC(C)[C@@H](N)C(=O)N1CCN(c2ccc(Cl)cc2Cl)[C@H](c2ccc(Cl)cc2)C1] |
| 748 | 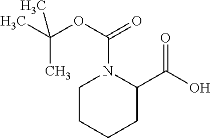 [SMILES: CC(C)(C)OC(=O)N1CCCCC1C(=O)O] | 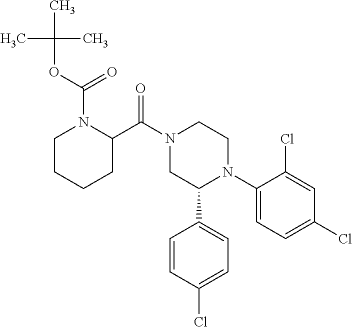 [SMILES: CC(C)(C)OC(=O)N1CCCCC1C(=O)N1CCN(c2ccc(Cl)cc2Cl)[C@H](c2ccc(Cl)cc2)C1] |
| 749 | 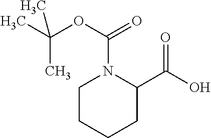 [SMILES: CC(C)(C)OC(=O)N1CCCCC1C(=O)O] | 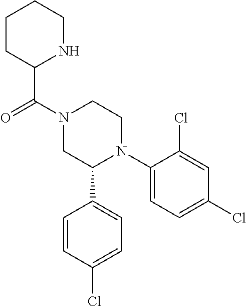 [SMILES: O=C(C1CCCCN1)N1CCN(c2ccc(Cl)cc2Cl)[C@H](c2ccc(Cl)cc2)C1] |
| 750 | 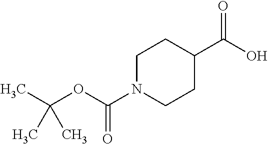 [SMILES: CC(C)(C)OC(=O)N1CCC(C(=O)O)CC1] | 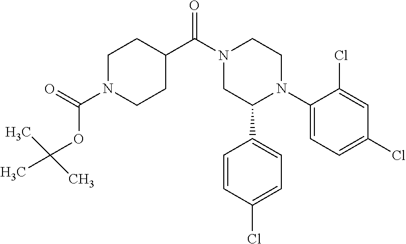 [SMILES: CC(C)(C)OC(=O)N1CCC(C(=O)N2CCN(c3ccc(Cl)cc3Cl)[C@H](c3ccc(Cl)cc3)C2)CC1] |
| 751 | 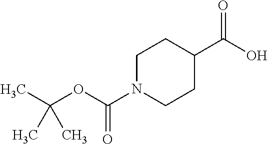 [SMILES: CC(C)(C)OC(=O)N1CCC(C(=O)O)CC1] | 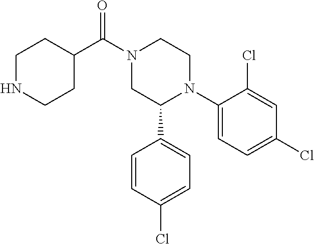 [SMILES: O=C(C1CCNCC1)N1CCN(c2ccc(Cl)cc2Cl)[C@H](c2ccc(Cl)cc2)C1] |
| 752 | 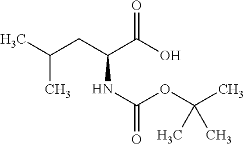 [SMILES: CC(C)C[C@H](NC(=O)OC(C)(C)C)C(=O)O] | 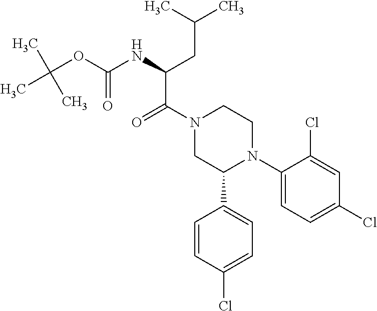 [SMILES: CC(C)C[C@H](NC(=O)OC(C)(C)C)C(=O)N1CCN(c2ccc(Cl)cc2Cl)[C@H](c2ccc(Cl)cc2)C1] |
| 753 | 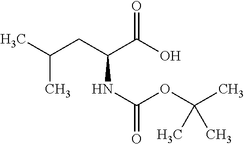 [SMILES: CC(C)C[C@H](NC(=O)OC(C)(C)C)C(=O)O] | 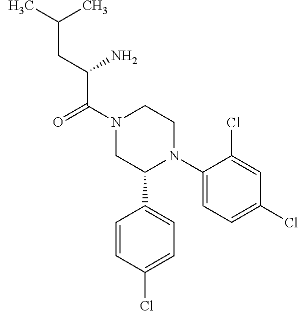 |
| 754 | 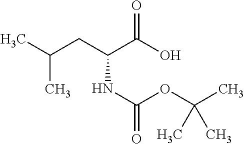 [SMILES: CC(C)C[C@@H](NC(=O)OC(C)(C)C)C(=O)O] | 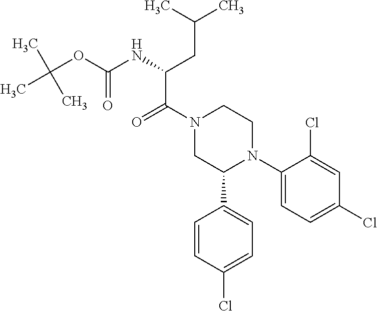 [SMILES: CC(C)C[C@@H](NC(=O)OC(C)(C)C)C(=O)N1CCN(c2ccc(Cl)cc2Cl)[C@H](c2ccc(Cl)cc2)C1] |
| 755 | 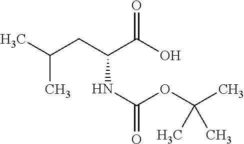 [SMILES: CC(C)C[C@@H](NC(=O)OC(C)(C)C)C(=O)O] | 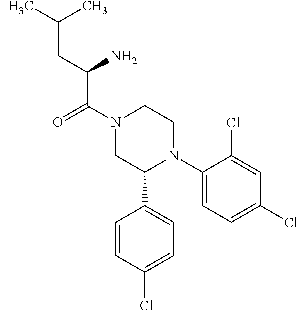 |
| 756 | 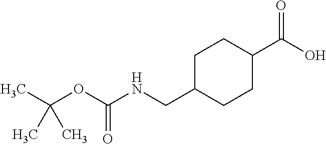 [SMILES: CC(C)(C)OC(=O)NCC1CCC(C(=O)O)CC1] | 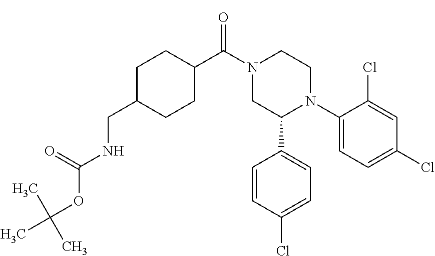 |
| 757 | 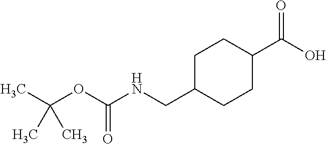 [SMILES: CC(C)(C)OC(=O)NCC1CCC(C(=O)O)CC1] | 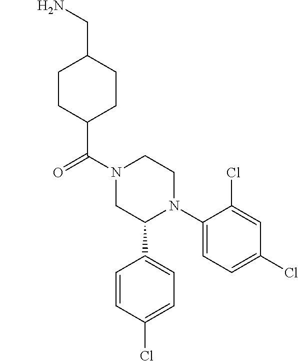 |
| 758 | 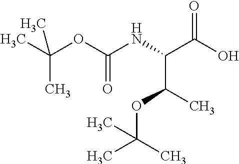 [SMILES: C[C@@H](OC(C)(C)C)[C@H](NC(=O)OC(C)(C)C)C(=O)O] | 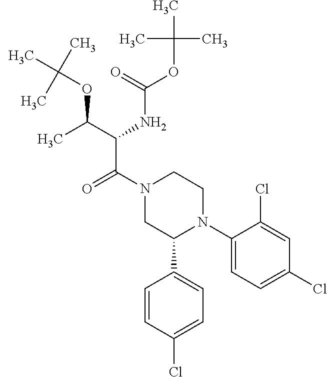 |
| 759 | 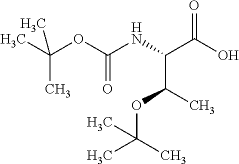 [SMILES: C[C@@H](OC(C)(C)C)[C@H](NC(=O)OC(C)(C)C)C(=O)O] | 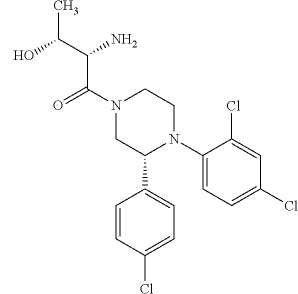 |
| 760 | 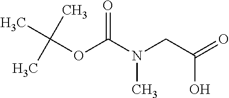 | 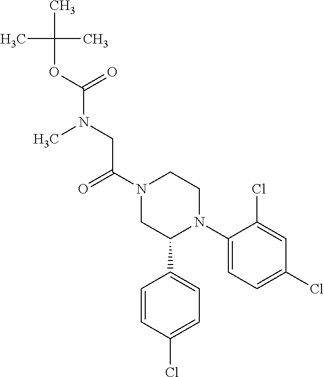 |
| 761 | 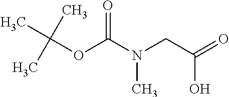 | 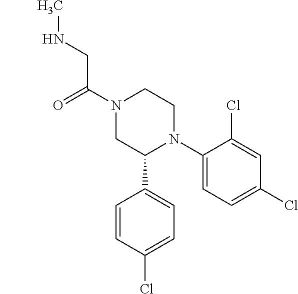 |
| 762 | 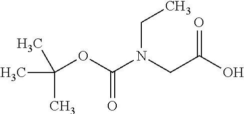 | 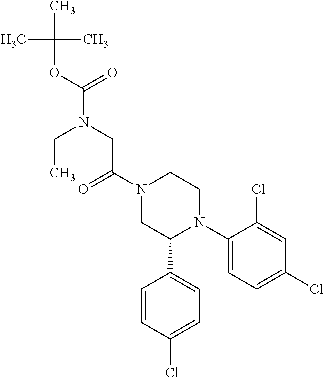 |
| 763 | 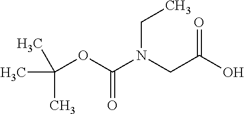 | 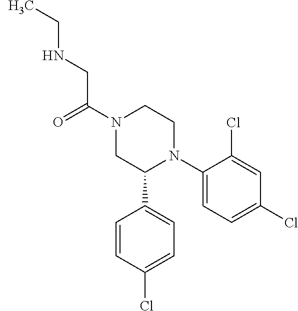 |
| 764 | 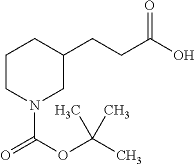 [SMILES: CC(C)(C)OC(=O)N1CCCC(CCC(=O)O)C1] | 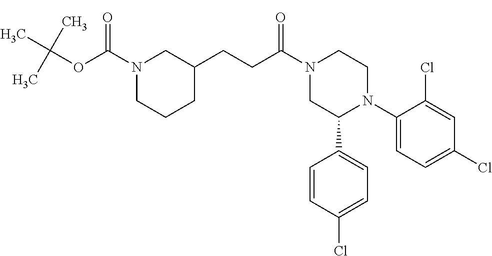 |
| 765 | 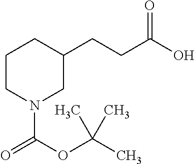 [SMILES: CC(C)(C)OC(=O)N1CCCC(CCC(=O)O)C1] | 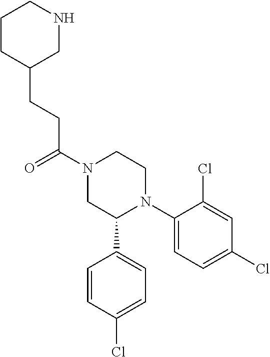 [SMILES: O=C(CCC1CCCNC1)N1CCN(c2ccc(Cl)cc2Cl)[C@H](c2ccc(Cl)cc2)C1] |
| 766 | 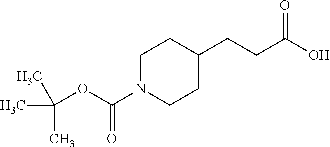 [SMILES: CC(C)(C)OC(=O)N1CCC(CCC(=O)O)CC1] | 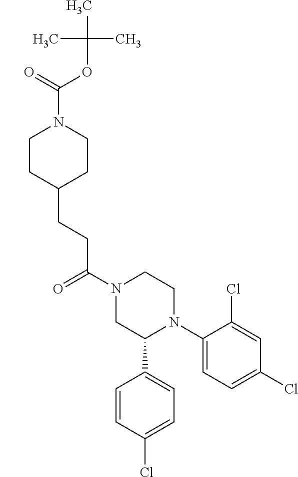 |
| 767 | 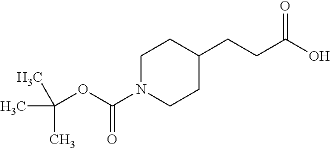 [SMILES: CC(C)(C)OC(=O)N1CCC(CCC(=O)O)CC1] | 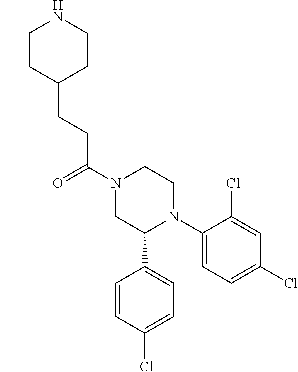 |

### Preparation of Example 768

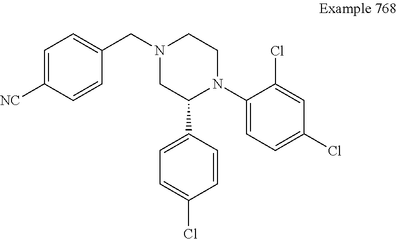 [SMILES: N#Cc1ccc(CN2CCN(c3ccc(Cl)cc3Cl)[C@H](c3ccc(Cl)cc3)C2)cc1]

Example 768 was prepared by the method of Scheme 1, except that Example 304 was used instead of Example 1 and 4-cyanobenzaldehyde was used instead of benzaldehyde.

### Preparation of Example 769

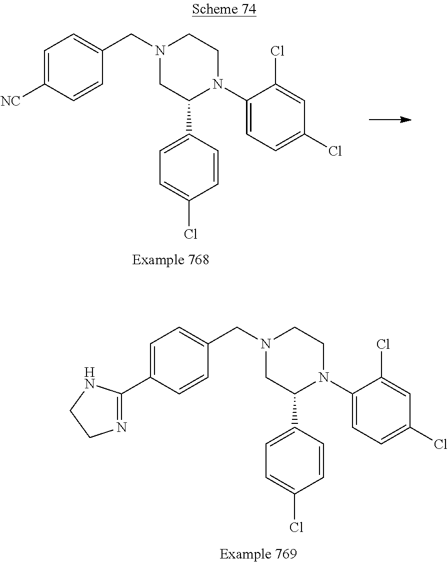 [SMILES: N#Cc1ccc(CN2CCN(c3ccc(Cl)cc3Cl)[C@H](c3ccc(Cl)cc3)C2)cc1 ; Clc1ccc([C@@H]2CN(Cc3ccc(C4=NCCN4)cc3)CCN2c2ccc(Cl)cc2Cl)cc1]

To the piperazine Example 768 (0.1 g, 0.22 mmol) in ethylenediamine (15 μL) was added Yttrium (III) trifluoromethane sulfonate (1.2 mg). The mixture was warmed to 100° C. and stirred for 20 h. An additional amount of ethylenediamine (0.2 mL) and Yttrium (III) trifluoromethane sulfonate (1 mg) was added, and the mixture was stirred at 100° C. for 4 days. Additional ethylenediamine (0.2 mL) and Yttrium (III) trifluoromethane sulfonate (1 mg) were then added, and the mixture stirred for an additional 24 h at 100° C. The reaction mixture was cooled to room temperature and purified by preparative TLC (25% EtOAc/hexane) to provide

Example 769 (0.022 g).

### Preparation of Example 770

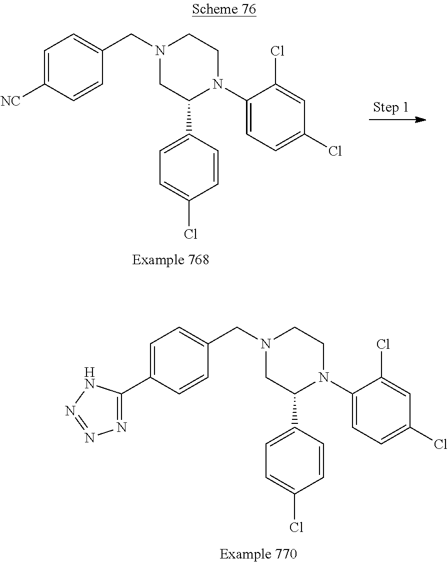 [SMILES: N#Cc1ccc(CN2CCN(c3ccc(Cl)cc3Cl)[C@H](c3ccc(Cl)cc3)C2)cc1 ; Clc1ccc([C@@H]2CN(Cc3ccc(-c4nnn[nH]4)cc3)CCN2c2ccc(Cl)cc2Cl)cc1]

### Step 1:

To the piperazine Example 768 (0.1 g, 0.22 mmol) in DMF (2.2 mL) in a microwave reactor vial was added NaN₃ (0.17 g, 2.6 mmol) and NH₄Cl (0.14 g, 2.6 mmol). The reaction mixture was capped and irradiated in a microwave reactor at 20 W. The reaction temperature reached ˜220° C. The reaction was irradiated for 25 minutes and cooled to room temperature. Saturated NaHCO₃ was added and the mixture was extracted with EtOAc. The organic layer was washed with water and brine. The organic layer was then dried (MgSO₄), filtered, and concentrated in vacuo. The residue was purified by silica gel preparative TLC (5% MeOH/CH₂Cl₂) to provide Example 770 (0.035 g).

### Preparation of Examples 771-773

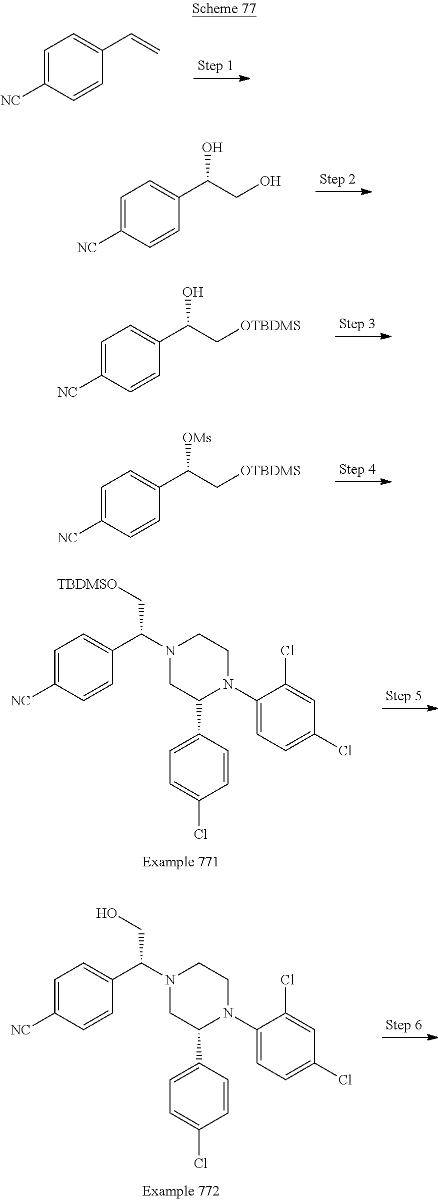

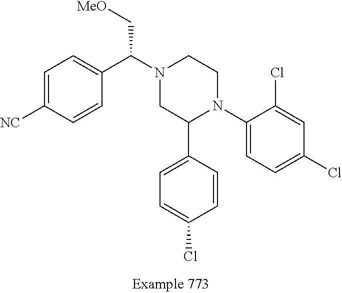

### Step 1

To AD mix α (available from Aldrich) (10.8 g) in tert-butyl alcohol/water (1:1) (78 mL) at 0° C. was added 4-cyanostyrene (1.0 g, 7.7 mmol). The reaction was stirred for 20 h, allowing the cold bath to expire. The reaction was cooled to 0° C. and solid sodium sulfite (10 g) was added. The mixture was allowed to warm to room temperature while stirring for 1 h. The mixture was then extracted with EtOAc. The organic layer was washed with water and brine, dried (MgSO₄), filtered, and concentrated in vacuo. The residue was purified by silica gel chromatography (5% MeOH/CH₂Cl₂) to provide the corresponding diol (1.24 g).

### Step 2

To the diol prepared in step 1 (0.62 g, 3.8 mmol) in DMF (10 mL) at 0° C. was added imidazole (0.65 g, 9.5 mmol) followed by TBDMS-Cl (i.e., tert-butyldimethylsilyl chloride) (0.69 g, 4.6 mmol). The reaction mixture was stirred for 4 h while warming to room temperature. The reaction mixture was poured into brine and then extracted with EtOAc. The organic layer was washed with water, brine, dried (MgSO₄), filtered, and concentrated in vacuo. The residue was purified by silica gel chromatography (20% EtOAc/hexane) to provide a tert-butyldimethylsilyl ether (0.67 g).

### Step 3

To the tert-butyldimethylsilyl ether prepared in step 2 (0.67 g, 2.4 mmol) in CH₂Cl₂ (8 mL) at 0° C. was added TEA (i.e., triethylamine) (0.5 mL, 3.6 mmol) followed by MeSO₂Cl (0.22 mL, 2.9 mmol). The reaction mixture was stirred for 2 h and CH₂Cl₂ was added. The mixture was washed with saturated NaHCO₃₀₀ water, and brine. The organic layer was dried (MgSO₄), filtered, and concentrated in vacuo to provide a methylsulfonyl ester (0.87 g) that was used directly in step 4 without further purification.

### Step 4

To the methylsulfonyl ester prepared in step 3 (0.34 g, 1 mmol) in acetonitrile (3 mL) was added piperazine Example 304 (0.44 g, 1.25 mmol) and potassium carbonate (0.35 g, 2.5 mmol). The reaction mixture was warmed to reflux and stirred for 24 h. The reaction was cooled to room temperature and EtOAc was added. The mixture was washed with water and brine. The organic layer was dried (MgSO₄), filtered, and concentrated in vacuo. The residue was purified by silica gel chromatography (20% EtOAc/hexane) to provide Example 771 (0.40 g).

### Step 5

To Example 771 (0.40 g, 0.66 mmol) in THF (2 mL) at 0° C. was added tetrabutylammonium fluoride (1.0 M in THF, 0.74 mL). The reaction was stirred for 20 h while warming to room temperature. EtOAc was added and the mixture washed with water and brine. The organic layer was dried (MgSO₄), filtered, and concentrated in vacuo. The residue was purified by silica gel chromatography (60% EtOAc/hex) to provide Example 772 (0.31 g, 0.64 mmol).

### Step 6

To Example 772 (0.15 g, 0.31 mmol) in THF at room temperature was added NaH (15 mg) (60% dispersion in mineral oil) followed by methyl iodide (0.024 mL, 0.38 mmol.). The reaction was stirred at room temperature for 20 h. Water was added slowly and the mixture was extracted with EtOAc. The organic layer was washed with water, brine, dried (MgSO₄), filtered, and concentrated in vacuo. The residue was purified by silica gel chromatography (30% EtOAc/hexane) to provide Example 773 (0.14 g).

### Preparation of Example 774

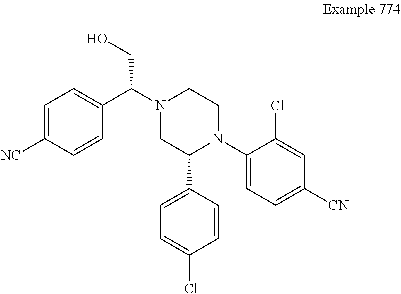 [SMILES: N#Cc1ccc([C@H](CO)N2CCN(c3ccc(C#N)cc3Cl)[C@H](c3ccc(Cl)cc3)C2)cc1]

Example 774 was prepared using procedures similar to those used to prepare Example 772, except that the piperazine Example 305 was used instead of Example 304 in step 4 of Scheme 77.

### Preparation of Example 775

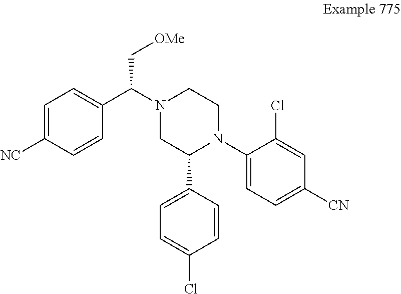 [SMILES: COC[C@@H](c1ccc(C#N)cc1)N1CCN(c2ccc(C#N)cc2Cl)[C@H](c2ccc(Cl)cc2)C1]

Example 775 was prepared using procedures similar to those used to prepare Example 773, except that the piperazine Example 305 was used instead of Example 304 in step 4 of Scheme 77.

### Preparation of Example 776a and 776b

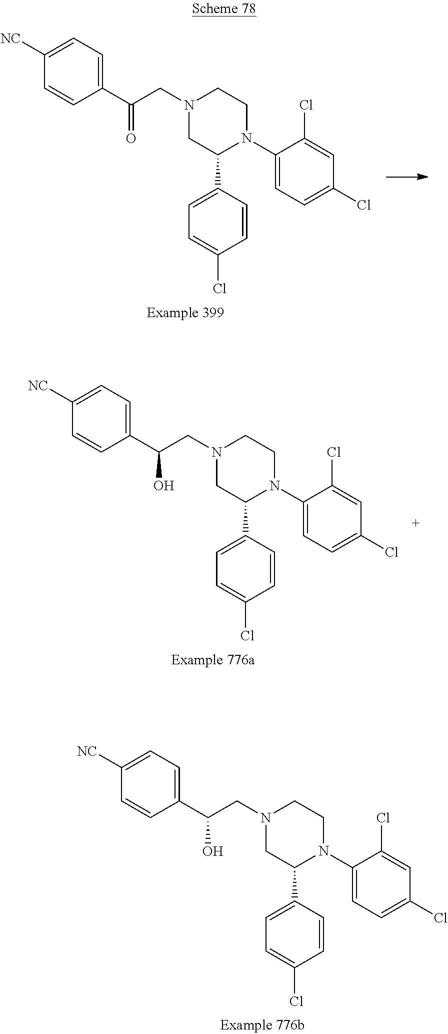 [SMILES: N#Cc1ccc(C(=O)CN2CCN(c3ccc(Cl)cc3Cl)[C@H](c3ccc(Cl)cc3)C2)cc1 ; N#Cc1ccc([C@H](O)CN2CCN(c3ccc(Cl)cc3Cl)[C@H](c3ccc(Cl)cc3)C2)cc1 ; N#Cc1ccc([C@@H](O)CN2CCN(c3ccc(Cl)cc3Cl)[C@H](c3ccc(Cl)cc3)C2)cc1]

To Example 399 (0.16 g, 0.32 mmol) in MeOH (1.5 mL) at 0° C. was added NaBH₄ (0.012 g, 0.32 mmol) (with gas evolution). The cold bath was removed from the reaction vessel, and the reaction mixture was stirred for 45 minutes. The reaction mixture was concentrated in vacuo. Water was added and the reaction mixture was extracted with EtOAc. The organic layers were combined and washed with water, brine, dried (MgSO₄), filtered, and concentrated in vacuo. The residue was purified by silica gel chromotography (5-50% EtOAc/hexane) to provide the corresponding alcohol as a mixture of diastereomers (0.8 g, 0.16 mmol). The diastereomers were separated by chiral prep HPLC (Chiralcel OD, 85% hexane/IPA, 50 mL/min, 254 nm) to provide Examples 776a and 776b.

Alternatively, Example 776a was prepared by the following method:

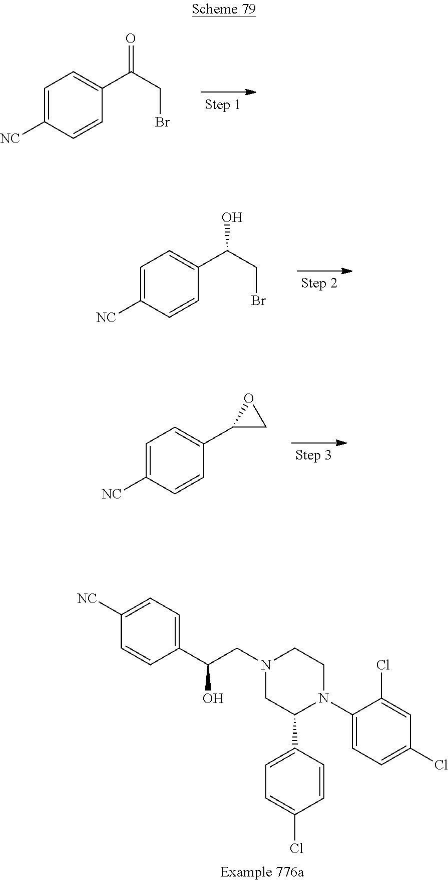 [SMILES: N#Cc1ccc(C(=O)CBr)cc1 ; N#Cc1ccc([C@H](O)CBr)cc1 ; N#Cc1ccc([C@H]2CO2)cc1 ; N#Cc1ccc([C@H](O)CN2CCN(c3ccc(Cl)cc3Cl)[C@H](c3ccc(Cl)cc3)C2)cc1]

### Step 1:

To 2-bromo-4′-cyanoacetophenone (1.0 g, 4.5 mmol) in THF (4.5 mL) at 0° C. was added (S)-2-methyl-CBS-oxazaborolidine (1 M in toluene, 0.89 mL) followed by BH₃.SMe₂ (2.0M in THF, 1.3 mL). The mixture was stirred at 0° C. for 75 minutes. MeOH (−5 mL) was added (with gas evolution) and the mixture was stirred for 15 minutes. The reaction mixture was concentrated in vacuo. The residue was taken up into CH₂Cl₂ and washed with 1N HCL, water, and brine, dried (MgSO₄), filtered, and concentrated in vacuo to provide the corresponding alcohol which was used directly in the next step without further purification.

### Step 2:

The alcohol prepared in step 1 was taken up into toluene (40 mL). 1N NaOH (40 mL) was added and the mixture was stirred at room temperature for 20 h. The organic layer was washed with water and brine, dried (MgSO₄), filtered, and concentrated in vacuo. The resulting residue was purified by silica gel chromatography (0-20% EtOAc/hexane) to provide the epoxide (0.52 g, 3.6 mmol).

### Step 3:

To the piperazine Example 304 (0.15 g, 0.44 mmol) was added the epoxide (0.07 g, 0.48 mmol) prepared in step 2. The reaction mixture was heated neat (without solvent) at 100° C. for 18 h. The reaction was cooled to room temperature and the reaction mixture purified by silica gel chromatography (5-40% EtOAc/hexanes) to provide a residue (0.14 g) which was further purified by chiral preparative HPLC [Chiralcel OD 85% hexanes/IPA, 50 mL/min, 254 nm] to provide Example 776a (0.13 g, 0.29 mmol).

### Preparation of Example 777

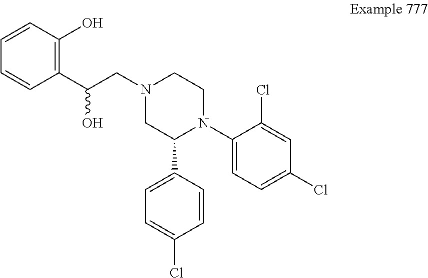 [SMILES: Oc1ccccc1C(O)CN1CCN(c2ccc(Cl)cc2Cl)[C@H](c2ccc(Cl)cc2)C1]

Example 777 was prepared from the ketone Example 780 by reduction with sodium borohydride as described above in Scheme 78.

### Preparation of Example 778

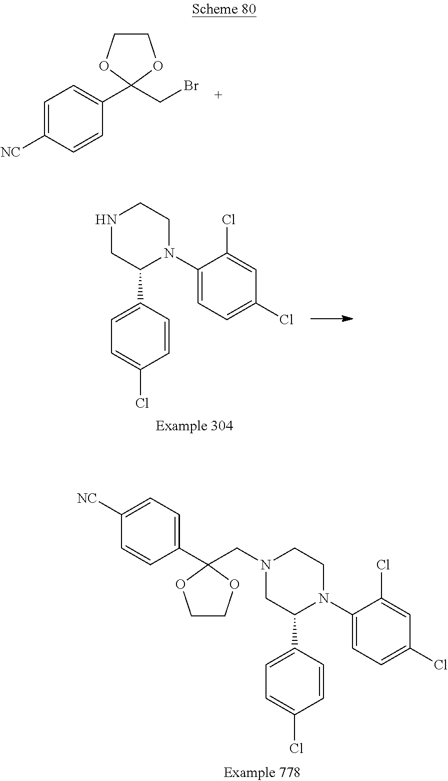 [SMILES: N#Cc1ccc(C2(CBr)OCCO2)cc1 ; Clc1ccc([C@@H]2CNCCN2c2ccc(Cl)cc2Cl)cc1 ; N#Cc1ccc(C2(CN3CCN(c4ccc(Cl)cc4Cl)[C@H](c4ccc(Cl)cc4)C3)OCCO2)cc1]

To the piperazine Example 304 (0.15 g, 0.44 mmol) in DMF (1.5 mL) was added potassium carbonate (0.12 g, 0.88 mmol) and 4-[2-(bromomethyl)-1,3-dioxolane-2-yl]benzonitrile (0.15 g, 0.55 mmol) followed by sodium iodide (0.025 g, 0.16 mmol). The reaction mixture was warmed to 150° C. and stirred for 4 days. The reaction mixture was then cooled to room temperature and water was added. The resulting mixture was extracted with EtOAc. The combined organic layers were washed with water, brine, dried (MgSO₄), filtered, and concentrated in vacuo. The residue was purified by silica gel chromotography (0-25% EtOAc/hex) to provide the ketal Example 778 (0.10 g).

### Preparation of Example 779

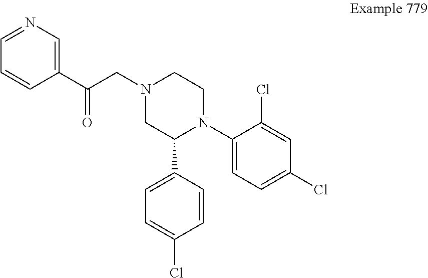 [SMILES: O=C(CN1CCN(c2ccc(Cl)cc2Cl)[C@H](c2ccc(Cl)cc2)C1)c1cccnc1]

Example 779 was prepared in the same manner as Example 398 in scheme 69 except that 2-bromo-1-pyridin-3-ylethan-1-one hydrobromide was used instead of 2-bromoacetophenone.

### Preparation of Example 780

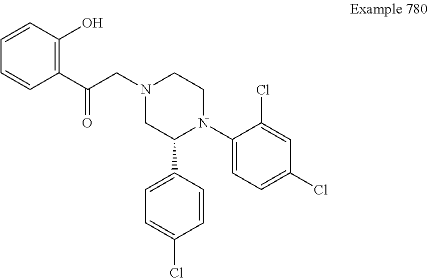 [SMILES: O=C(CN1CCN(c2ccc(Cl)cc2Cl)[C@H](c2ccc(Cl)cc2)C1)c1ccccc1O]

Example 780 was prepared in the same manner as Example 398 in scheme 69 except that 2-bromo-2′-hydroxyacetophenone was used instead of 2-bromoacetophenone.

### Preparation of Example 781

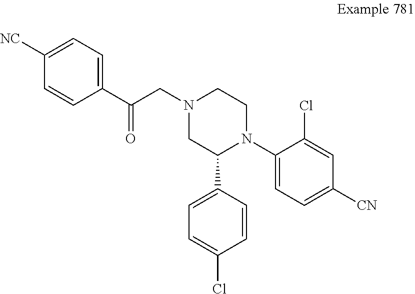 [SMILES: N#Cc1ccc(C(=O)CN2CCN(c3ccc(C#N)cc3Cl)[C@H](c3ccc(Cl)cc3)C2)cc1]

The ketone Example 781 was prepared in the same manner as Example 399, except that the piperazine Example 305 was used instead of Example 304.

### Preparation of Examples

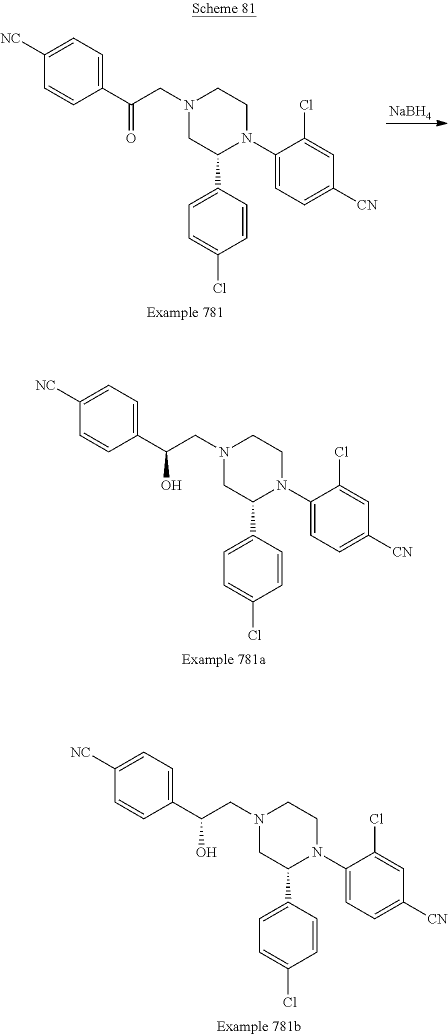 [SMILES: N#Cc1ccc(C(=O)CN2CCN(c3ccc(C#N)cc3Cl)[C@H](c3ccc(Cl)cc3)C2)cc1 ; "NaBH4" ; N#Cc1ccc([C@H](O)CN2CCN(c3ccc(C#N)cc3Cl)[C@H](c3ccc(Cl)cc3)C2)cc1 ; N#Cc1ccc([C@@H](O)CN2CCN(c3ccc(C#N)cc3Cl)[C@H](c3ccc(Cl)cc3)C2)cc1]

Example 781 was reduced with NaBH₄ using the procedure described for the reduction of Example 399 in Scheme 78 to provide a mixture of diasteromers (781a and 781b) that were separated by chiral preparative HPLC [Chiralcel OD, 25% IPA/hexane, 50 mL/min., 254 nm].

### Preparation of Example 782-786

Examples 782-786 were prepared according to the method of Scheme 38, using the appropriate piperazine and alcohol shown in Table XXII, below.

TABLE XXII

| Example # | Piperazine | Alcohol | Structure |
|---|---|---|---|
| 782 | 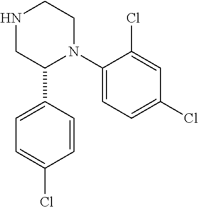 | 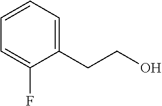 | 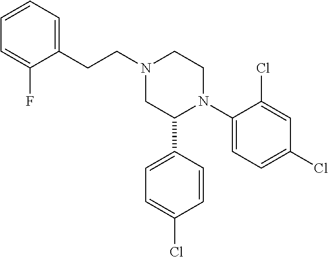 |
| 783 | 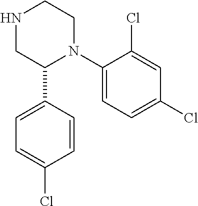 | 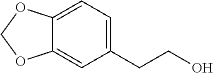 [SMILES: OCCc1ccc2c(c1)OCO2] | 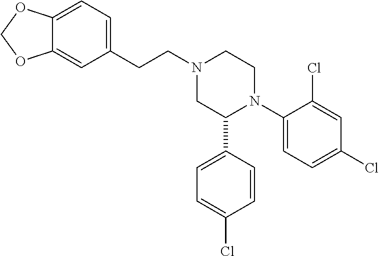 |
| 784 | 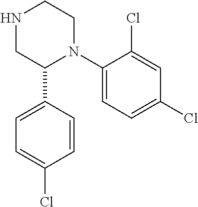 | 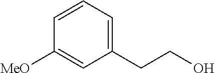 [SMILES: COc1cccc(CCO)c1] | 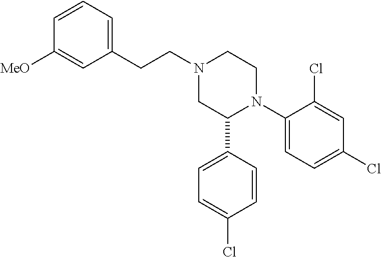 |
| 785 | 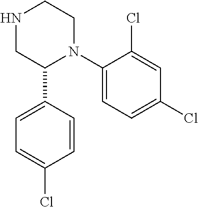 | 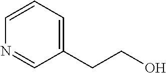 | 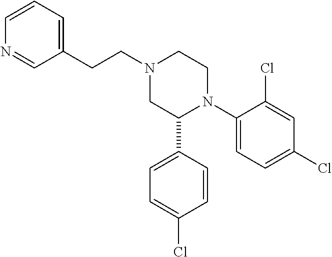 |
| 786 | 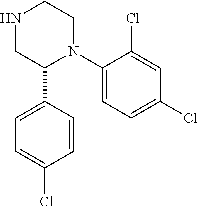 | 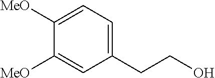 [SMILES: COc1ccc(CCO)cc1OC] | 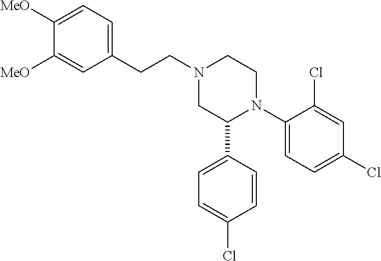 |

### Preparation of Examples 787-792

Using the procedures outlined in Scheme 41, the Amide and Amine Examples 787-792 were prepared from the piperazine Example 304 and the carboxylic acid indicated in Table XXIII, below.

TABLE XXIII

| Acid | Amide | Amine |
|---|---|---|
| 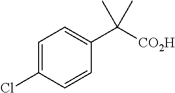 | 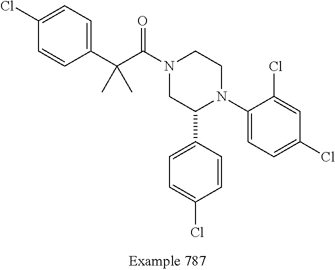 | 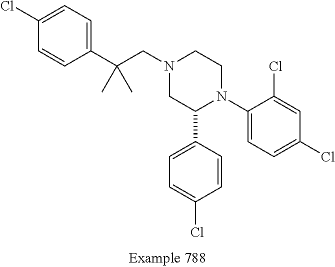 |
| 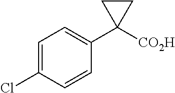 [SMILES: O=C(O)C1(c2ccc(Cl)cc2)CC1] | 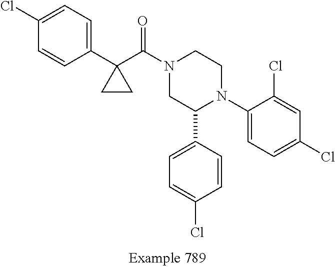 | 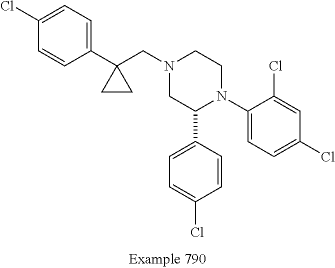 |
| 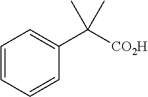 | 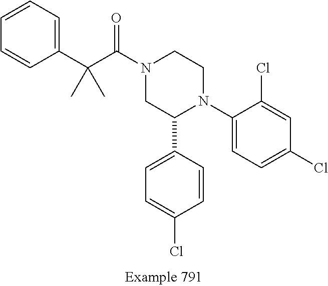 | 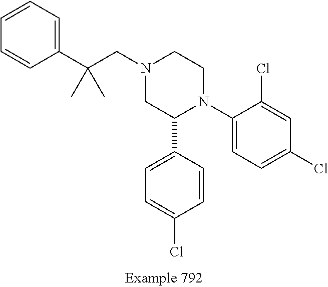 |

### Preparation of Example 793

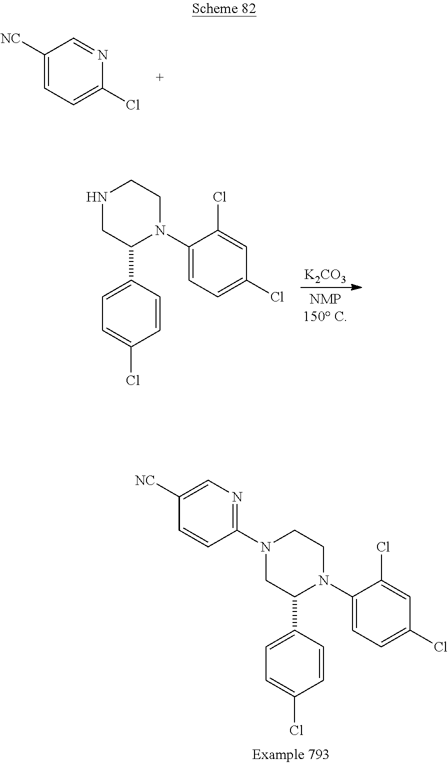

2-Chloro-5-cyano-pyridine (56 mg), the piperazine Example 304 (125 mg), and K₂CO₃ (100 mg) were taken up in 1-methyl-2-pyrrolidinone (NMP) and heated at 120° C. for 18 hours. The reaction mixture was heated at 150° C. for 5 hours. The reaction mixture was cooled and partitioned between EtOAc and saturated NaHCO_{3(aq.)}. The aqueous layer was extracted with EtOAc. The combined organic layers were washed with brine and dried (MgSO₄). Filtration and concentration gave a yellow oil. Purification via thin-layer preparative chromatography (1.2/1 CH₂Cl₂/hexanes, SiO₂) furnished 110 mg (63%) of Example 793 as a colorless oil.

### Preparation of Example 794

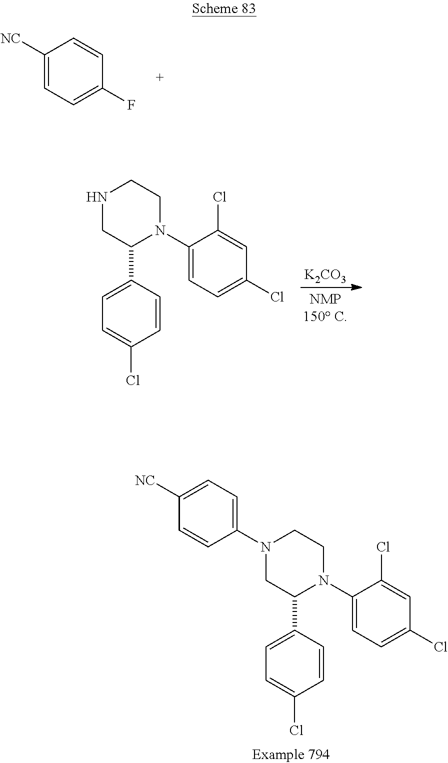

Example 794 was prepared according to the procedure outlined above in Scheme 82 using 4-fluoro-benzonitrile in place of 2-chloro-5-cyano-pyridine.

### Preparation of Examples 795-796

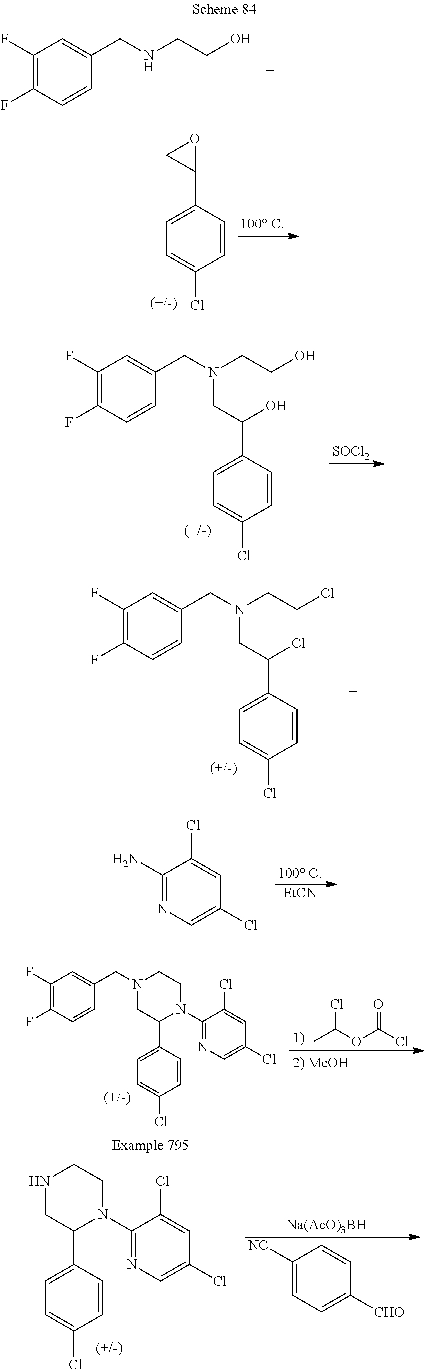

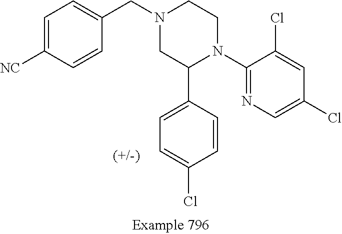 [SMILES: N#Cc1ccc(CN2CCN(c3ncc(Cl)cc3Cl)C(c3ccc(Cl)cc3)C2)cc1]

Example 795 was prepared according to the procedures outlined in Steps 2, 3, and 4 of Scheme 7 using 2-(4-chlorophenyl)oxirane and 2-amino-3,5-dichloropyridine as indicated in Scheme 84, above. Example 796 was prepared from Example 795 using the procedures outlined in Steps 5 and 6 of Scheme 24.

### Preparation of Examples 797, 797a, and 797b

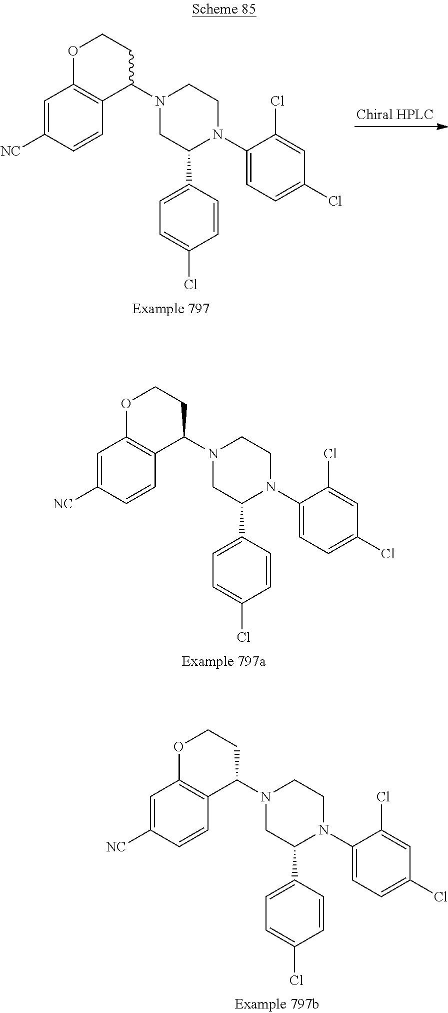 [SMILES: N#Cc1ccc2c(c1)OCCC2N1CCN(c2ccc(Cl)cc2Cl)[C@H](c2ccc(Cl)cc2)C1 ; N#Cc1ccc2c(c1)OCC[C@H]2N1CCN(c2ccc(Cl)cc2Cl)[C@H](c2ccc(Cl)cc2)C1 ; N#Cc1ccc2c(c1)OCC[C@@H]2N1CCN(c2ccc(Cl)cc2Cl)[C@H](c2ccc(Cl)cc2)C1]

Example 797 was prepared in the same manner as Example 397 in Scheme 68 except that 7-cyano-4-chromanone was used instead of 4-chromanone. The 1:1 mixture of diastereomers of Example 797 was separated by chiral preparative HPLC [Chiralcel OD, 10% IPA/hexane, 40 mL/min, 254 nm] to provide a faster eluting and a slower eluting isomer.

### Preparation of Examples 798, 798a, and 798b

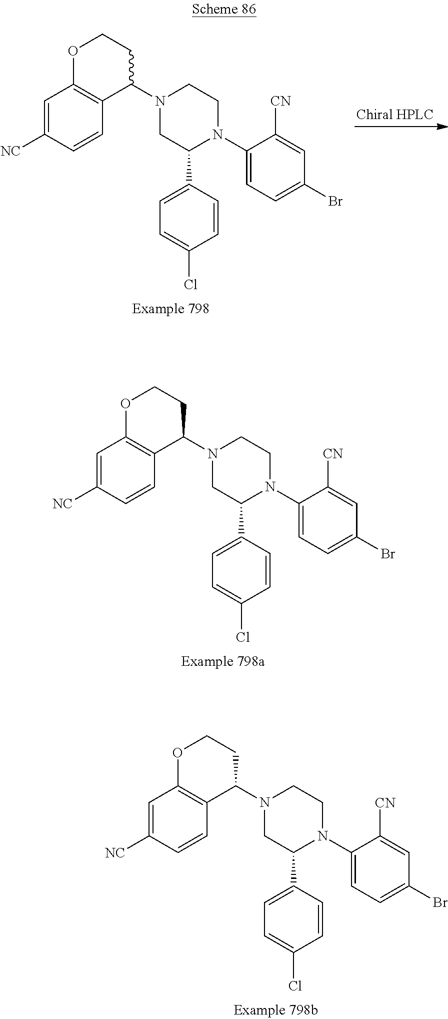 [SMILES: N#Cc1ccc2c(c1)OCCC2N1CCN(c2ccc(Br)cc2C#N)[C@H](c2ccc(Cl)cc2)C1 ; N#Cc1ccc2c(c1)OCC[C@H]2N1CCN(c2ccc(Br)cc2C#N)[C@H](c2ccc(Cl)cc2)C1 ; N#Cc1ccc2c(c1)OCC[C@@H]2N1CCN(c2ccc(Br)cc2C#N)[C@H](c2ccc(Cl)cc2)C1]

Example 798 was prepared in the same manner as Example 397 in scheme 68 except that 7-cyano-4-chromanone was used instead of 4-chromanone in step 1 and Example 309 was used instead of Example 304 in step 3. The 1:1 mixture of diastereomers of Example 798 was separated by chiral preparative HPLC [Chiralcel OD, 20% IPA/hexane, 45 ml/min, 254 nm] to provide a faster eluting and a slower eluting isomer.

### Preparation of Examples 799, 799a, and 799b

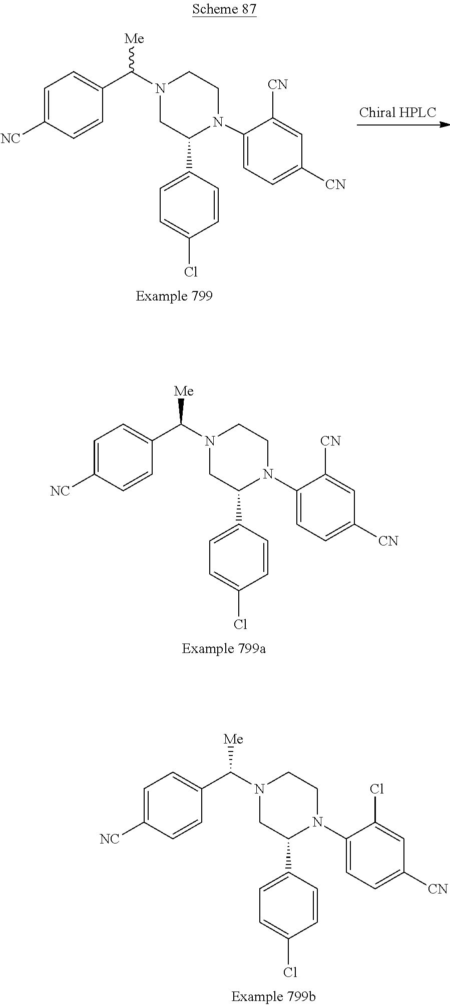 [SMILES: CC(c1ccc(C#N)cc1)N1CCN(c2ccc(C#N)cc2C#N)[C@H](c2ccc(Cl)cc2)C1 ; C[C@H](c1ccc(C#N)cc1)N1CCN(c2ccc(C#N)cc2C#N)[C@H](c2ccc(Cl)cc2)C1 ; C[C@@H](c1ccc(C#N)cc1)N1CCN(c2ccc(C#N)cc2Cl)[C@H](c2ccc(Cl)cc2)C1]

Example 799 was prepared in the same manner as Example 391 in scheme 65 except that Example 309 was used instead of Example 304 in step 3. The 1:1 mixture of diastereomers of Example 799 was separated by chiral preparative HPLC [Chiralcel OD, 25% IPA/hexane, 50 mL/min, 254 nm] to provide a faster eluting and a slower eluting isomer.

### Preparation of Example 800

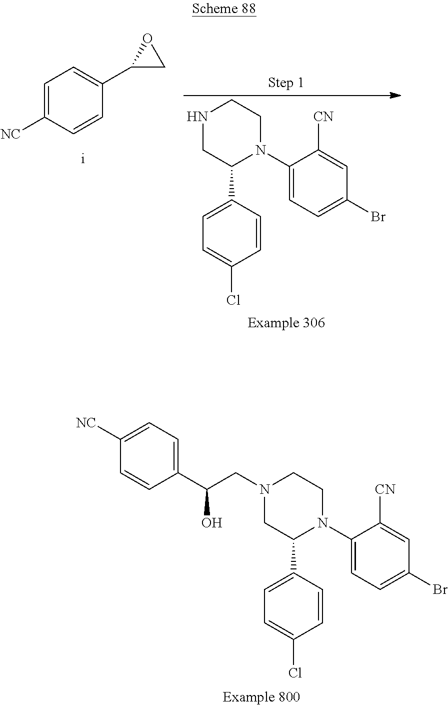 [SMILES: N#Cc1ccc([C@H]2CO2)cc1 ; N#Cc1cc(Br)ccc1N1CCNC[C@H]1c1ccc(Cl)cc1 ; N#Cc1ccc([C@H](O)CN2CCN(c3ccc(Br)cc3C#N)[C@H](c3ccc(Cl)cc3)C2)cc1]

### Step 1:

The epoxide i was prepared in the same manner as iii in Scheme 28 except that 2-bromo-4′-cyanoacetophenone was used instead of 2-bromo-4′-chloroacetophenone and (S)—CBS-oxazaborolidine was used instead of (R)—CBS-oxazaborolidine in step 1. The epoxide i (0.05 g, 0.36 mmol) was heated neat (without solvent) with the piperazine Example 306 (0.13 g, 0.33 mmol) at 100° C. for 18 h. The reaction mixture was cooled to room temperature and purified by silica gel chromatography (60% EtOAc/hexane). The residue, which contained a small amount of the minor diastereomer, was purified by chiral preparative HPLC [Chiralcel OD, 20% IPA/hexane, 50 mL/min, 254 nm] to provide Example 800 (0.11 g).

### Preparation of Example 801

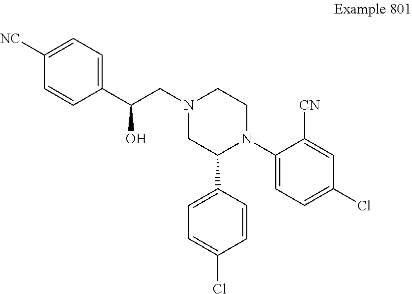 [SMILES: N#Cc1ccc([C@H](O)CN2CCN(c3ccc(Cl)cc3C#N)[C@H](c3ccc(Cl)cc3)C2)cc1]

Example 801 was prepared in the same manner as Example 800 except that Example 305 was used instead of Example 306 in Scheme 88.

### Preparation of Examples 802, 802a, and 802b

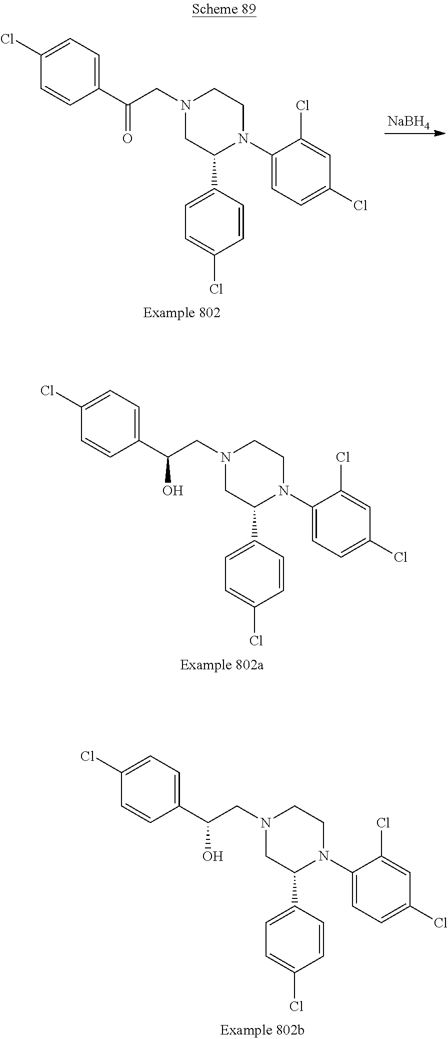 [SMILES: O=C(CN1CCN(c2ccc(Cl)cc2Cl)[C@H](c2ccc(Cl)cc2)C1)c1ccc(Cl)cc1 ; "NaBH4" ; O[C@H](CN1CCN(c2ccc(Cl)cc2Cl)[C@H](c2ccc(Cl)cc2)C1)c1ccc(Cl)cc1 ; O[C@@H](CN1CCN(c2ccc(Cl)cc2Cl)[C@H](c2ccc(Cl)cc2)C1)c1ccc(Cl)cc1]

The ketone Example 802 was prepared in the same manner as Example 398 in Scheme 69 except that 2-bromo-4′-cyanoacetophenone was used instead of 2-bromoacetophenone. Example 802a and Example 802b were prepared in the same manner as Example 776a and Example 776b in Scheme 78 except that Example 802 was used instead of Example 399.

Method for Evaluating Cannabinoid CB₁ and CB affinity

Competition binding assays for cannabinoid CB₁ and CB₂ affinity were performed by incubating commercially purchased membranes prepared from cells expressing each receptor subtype (8 μg pro) with 0.5 nM ³H-CP55,940, a non-selective cannabinoid agonist, along with concentrations of drug ranging from 0.0001-3 μM in Buffer A (5 mM MgCl₂, 2.5 mM EDTA and 013% BSA). Non-specific binding was defined in the presence of 10 μM CP55,940. For saturation studies, concentrations of ³H-CP55,940 ranging from 0.1-5 nM were incubated with membranes in the presence and absence of 10 μM CP55,940. Assays were terminated after incubation for 1½ hours by rapid filtration onto 0.3 polyethylenamine treated GF/C filterplates using a BRANDEL cell harvester. The plates were dried and MICROSCINT scintillation cocktail was added, after which the bound radioactivity was quantified using a TOPCOUNT scintillation counter.

The dissociation constant (K_{d}) of ³H-CP55,940 at the CB₁ and CB₂ receptor were determined by plotting specific binding at each concentration of radioligand, and analysis by non-linear regression. For competition studies, the concentration of each drug that inhibited 50 percent of ³H-CP55,940 binding (IC₅₀) was determined by non-linear regression analysis of the radioligand displacement curves. Affinity constants (Kᵢ) were calculated using the equation derived by Cheng and Prusoff (1973), defined as: IC₅₀/1+[conc. ligand/K_{d}].

### GTPγS Binding Protocol

The functional efficacy of compounds to activate second messengers within the cell was determined utilizing the GTPγS binding assay. Guanine nucleotides are phosphorylated within the plasma membrane of the cell following binding and activation by agonists. A radiolabelled derivative of guanine triphosphate (GTP) is utilized in this assay as it cannot be dephosphorylated and therefore accumulates following agonist binding. The simultaneous presence of an antagonist into this system will shift the agonist concentration curve to the right, with increasing concentrations of antagonist producing a greater rightward shift in the dose-response curve of the agonist.

Commercially purchased membranes were incubated with 10 mM GDP to allow sufficient substrate for phosphorylation in the presence of agonist. The membranes were then pre-incubated with increasing concentrations of test compound for 30 minutes to determine if they were capable of stimulating phosphorylation alone. Increasing concentrations of the non-selective cannabinoid agonist WIN55,122 were then added in the presence or absence of each concentration of test compound. The assay was then incubated for 1 hour at room temperature. To complete the assay, ³⁵S-GTPγS was added and the assay incubated for another 30 minutes. Assays were terminated by rapid filtration onto 10 mM sodium phosphate-treated GF/C filterplates using a Brandel cell harvester. The plates were dried and Microscint scintillation cocktail was added, after which the bound radioactivity was quantified using a Topcount scintillation counter.

The stimulation of ³⁵S-GTPγS binding as a function of the concentration of the agonist WIN55,122, in the absence and presence of test compound, was plotted and the EC₅₀ determined by nonlinear regression analysis using GraphPad Prism software. A Schild analysis of the rightward shift in the dose response curve of WIN55,122 in the presence of test compound was determined by plotting the concentration of test compound against the negative log of the dose ratio [1-(EC₅₀ agonist+test compound/EC50 of agonist alone)]. A linear regression analysis yields the Kb, defined as the X-intercept of the linear equation.

### Preparation of Compound of Formula (II)

Step 1): To a solution of (S)-4-phenyl-2-oxazolidinone (41 g, 0.25 mol) in CH₂Cl₂ (200 mL), was added 4-dimethylaminopyridine (2.5 g, 0.02 mol) and triethylamine (84.7 mL, 0.61 mol) and the reaction mixture was cooled to 0° C. Methyl-4-(chloroformyl)butyrate (50 g, 0.3 mol) was added as a solution in CH₂Cl₂ (375 mL) dropwise over 1 h, and the reaction was allowed to warm to 22° C. After 17 h, water and H₂SO₄ (2N, 100 mL), was added the layers were separated, and the organic layer was washed sequentially with NaOH (10%), NaCl (sat'd) and water. The organic layer was dried over MgSO₄ and concentrated to obtain a semicrystalline product.

Step 2): To a solution of TiCl₄ (18.2 mL, 0.165 mol) in CH₂Cl₂ (600 mL) at 0° C., was added titanium isopropoxide (16.5 mL, 0.055 mol). After 15 min, the product of Step 1 (49.0 g, 0.17 mol) was added as a solution in CH₂Cl₂ (100 mL). After 5 min., diisopropylethylamine (DIPEA) (65.2 mL, 0.37 mol) was added and the reaction mixture was stirred at 0° C. for 1 h, the reaction mixture was cooled to −20° C., and 4-benzyloxybenzylidine(4-fluoro)aniline (114.3 g, 0.37 mol) was added as a solid. The reaction mixture was stirred vigorously for 4 h at −20° C., then acetic acid was added as a solution in CH₂Cl₂ dropwise over 15 min, the reaction mixture was allowed to warm to 0° C., and H₂SO₄ (2N) was added. The reaction mixture was stirred an additional 1 h, the layers were separated, washed with water, separated and the organic layer was dried. The crude product was crystallized from ethanol/water to obtain the pure intermediate.

Step 3): To a solution of the product of Step 2 (8.9 g, 14.9 mmol) in toluene (100 mL) at 50° C., was added N,O-bis(trimethylsilyl)acetamide (BSA) (7.50 mL, 30.3 mmol). After 0.5 h, solid TBAF (0.39 g, 1.5 mmol) was added and the reaction mixture stirred at 50° C. for an additional 3 h. The reaction mixture was cooled to 22° C., CH₃OH (10 mL), was added. The reaction mixture was washed with HCl (1 N), NaHCO₃ (1 N) and NaCl (sat'd.), and the organic layer was dried over MgSO₄.

Step 4): To a solution of the product of Step 3 (0.94 g, 2.2 mmol) in CH₃OH (3 mL), was added water (1 mL) and LiOH.H₂O (102 mg, 2.4 mmole). The reaction mixture was stirred at 22° C. for 1 h and then additional LiOH.H₂O (54 mg, 1.3 mmole) was added. After a total of 2 h, HCl (1 N) and EtOAc was added, the layers were separated, the organic layer was dried and concentrated in vacuo. To a solution of the resultant product (0.91 g, 2.2 mmol) in CH₂Cl₂ at 22° C., was added ClC(O)C(O)Cl (0.29 mL, 3.3 mmol) and the mixture stirred for 16 h. The solvent was removed in vacuo.

Step 5): To an efficiently stirred suspension of 4-fluorophenylzinc chloride (4.4 mmol) prepared from 4-fluorophenylmagnesium bromide (1M in THF, 4.4 mL, 4.4 mmol) and ZnCl₂ (0.6 g, 4.4 mmol) at 4° C., was added tetrakis(triphenyl-phosphine)palladium (0.25 g, 0.21 mmol) followed by the product of Step 4 (0.94 g, 2.2 mmol) as a solution in THF (2 mL). The reaction was stirred for 1 h at 0° C. and then for 0.5 h at 22° C. HCl (1 N, 5 mL) was added and the mixture was extracted with EtOAc. The organic layer was concentrated to an oil and purified by silica gel chromatography to obtain 1-(4-fluorophenyl)-4(S)-(4-hydroxyphenyl)-3(R)-(3-oxo-3-phenylpropyl)-2-azetidinone:

HRMS calc'd for C₂₄H₁₉F₂NO₃=408.1429, found 408.1411.

Step 6): To the product of Step 5 (0.95 g, 1.91 mmol) in THF (3 mL), was added (R)-tetrahydro-1-methyl-3,3-diphenyl-1H,3H-pyrrolo-[1,2-c][1,3,2]oxazaborole (120 mg, 0.43 mmol) and the mixture was cooled to −20° C. After 5 min, borohydride-dimethylsulfide complex (2M in THF, 0.85 mL, 1.7 mmol) was added dropwise over 0.5 h. After a total of 1.5 h, CH₃OH was added followed by HCl (1 N) and the reaction mixture was extracted with EtOAc to obtain 1-(4-fluorophenyl)-3(R)-[3(S)-(4-fluorophenyl)-3-hydroxypropyl)]-4(S)-[4-(phenylmethoxy)phenyl]-2-azetidinone (compound 6A-1) as an oil. ¹H in CDCl₃ d H3=4.68. J=2.3 Hz. Cl (M+H) 500.

Use of (S)-tetra-hydro-1-methyl-3,3-diphenyl-1H,3H-pyrrolo-[1,2-c][1,3,2]oxazaborole gives the corresponding 3(R)-hydroxypropyl azetidinone (compound 6B-1). ¹H in CDCl₃ d H3=4.69. J=2.3 Hz. Cl (M+H) 500.

To a solution of compound 6A-1 (0.4 g, 0.8 mmol) in ethanol (2 mL), was added 10% Pd/C (0.03 g) and the reaction mixture was stirred under a pressure (60 psi) of H₂ gas for 16 h. The reaction mixture was filtered and the solvent was concentrated to obtain compound 6A. Mp 164-166° C.; Cl (M+H) 410.

[α]_{D}²⁵=−28.1° (c 3, CH₃OH) Elemental analysis calc'd for C₂₄H₂₁F₂NO₃: C 70.41; H, 5.17; N, 3.42; found C, 70.25; H, 5.19; N, 3.54.

Similarly treat compound 6B-1 to obtain compound 6B. Mp 129.5-132.5° C.; Cl (M+H) 410. Elemental analysis calc'd for C₂₄H₂₁F2NO₃: C, 70.41; H, 5.17; N, 3.42; found C, 70.30; H, 5.14; N, 3.52.

Step 6′ (Alternative): To a solution of the product of Step 5 (0.14 g, 0.3 mmol) in ethanol (2 mL), was added 10% Pd/C (0.03 g) and the reaction was stirred under a pressure (60 psi) of H₂ gas for 16 h. The reaction mixture was filtered and the solvent was concentrated to afford a 1:1 mixture of compounds 6A and 6B.

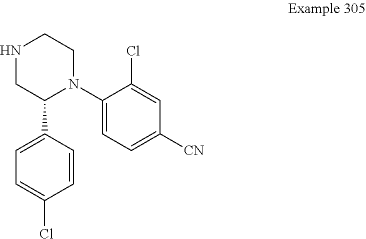 [SMILES: N#Cc1ccc(N2CCNC[C@H]2c2ccc(Cl)cc2)c(Cl)c1]

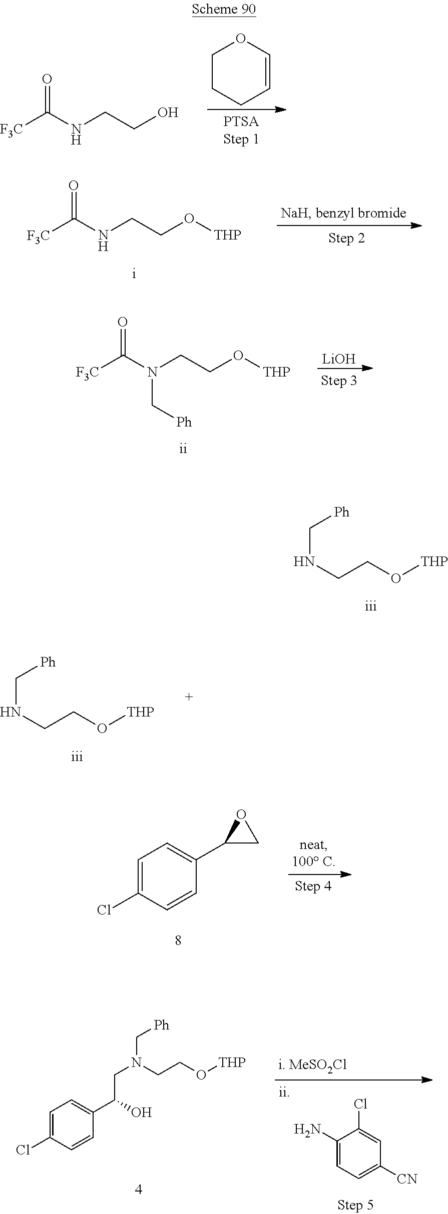

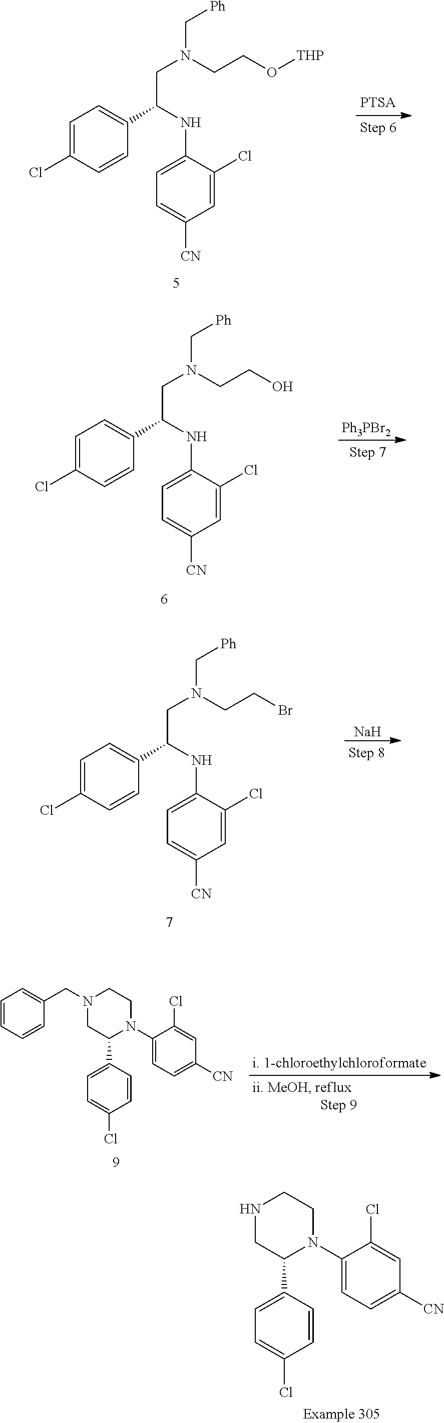

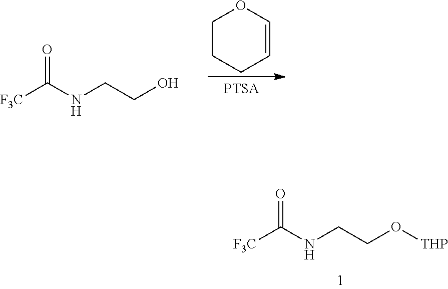

### Step 1

To N-(2-hydroxyethyl)-2,2,2-trifluoroacetamide (265 g, 1.68 mol) in CH₂Cl₂ (5.6 L) at room temperature was added 3,4-dihydro-2H-pyran (156 g, 1.86 mol) followed by p-toluenesulfonic acid (16 g, 0.084 mol). Stirred overnight at room temperature. Washed the reaction with saturated NaHCO₃, water, and brine. Dried (MgSO₄), filtered, and concentrated in vacuo to provide 1 (393 g, 1.63 mol) as a brown oil.

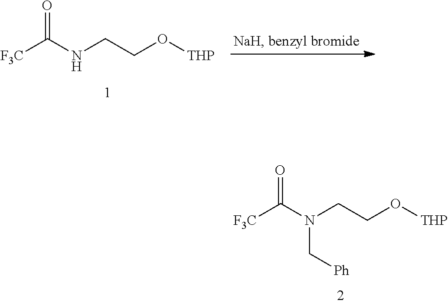

### Step 2

To 1 (393 g, 1.63 mol) in DMF (5.4 L) at 0° C. was added NaH (60% dispersion in mineral oil, 81.5 g, 2.04 mol) slowly over 35 minutes. The reaction was stirred for an additional 10 minutes and then the cold bath was removed. Continued stirring for 30 minutes and then added benzyl bromide (334 g, 1.95 mol). Heated overnight at 55° C. Cooled to room temperature and added 1.4 L of ice followed by 4 L of saturated brine. Extracted with ether. Combined the ether layers and washed with water. Dried (MgSO₄), filtered, and concentrated in vacuo. Purified by silica gel chromatography (3%, 4%, 5%, 10% EtOAc/hex) to provide 2 (505 g, 1.52 mol) as a colorless oil.

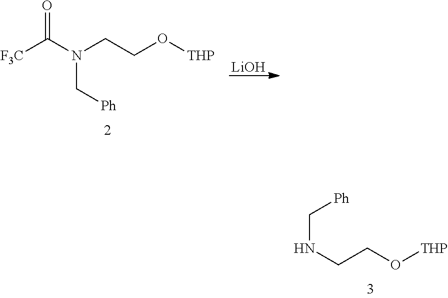

### Step 3

To 2 (500 g, 1.51 mmol) in THF (2.73 L) was added 2N LiOH (2.25 L). Stirred at room temperature overnight. Added water and extracted with EtOAc. Combined the organics and washed with water and brine. Dried (MgSO₄), filtered, and concentrated in vacuo. Purified by silica gel chromatography (2-10% MeOH/CH₂Cl₂) to provide 3 (263 g, 1.12 mol).

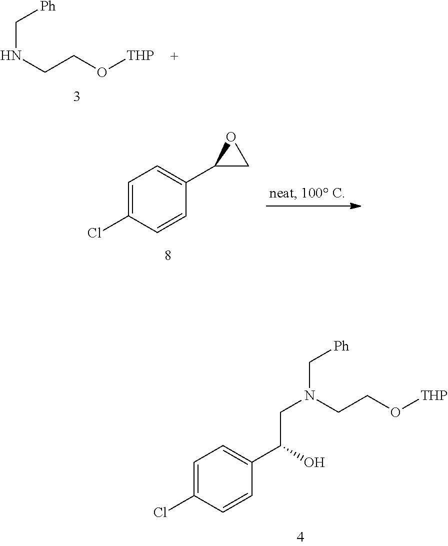

### Step 4

To 3 (200 g, 0.86 mol) was added 8 (140 g, 0.86 mol). Heated the reaction neat to 100° C. and stirred overnight. Cooled to room temperature and purified directly by silica gel chromatography (2-8% MeOH/CH₂Cl₂) to provide 4 (310 g, 0.79 mmol).

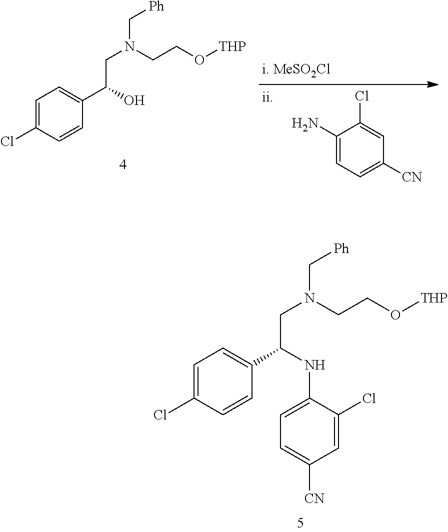

### Step 5

To 4 (160 g, 0.4 mol) in dichloroethane (1.3 L) at 0° C. was added TEA (139 mL, 1.0 mol) followed by MeSO₂Cl (37.5 mL, 0.48 mol). Allowed cold bath to expire over 2 h. Added the 4-amino-3-chlorobenzonitrile (76 g, 0.5 mol) and warmed to reflux. Stirred overnight at reflux and then cooled to room temperature. Added CH₂Cl₂ (4 L) and washed with saturated NaHCO₃, water, and brine. Dried (MgSO₄), filtered, and concentrated in vacuo. Purified by silica gel chromatography (10%, 13%, 16%, 20% EtOAc/hex) to provide 5 (178 g, 0.34 mol).

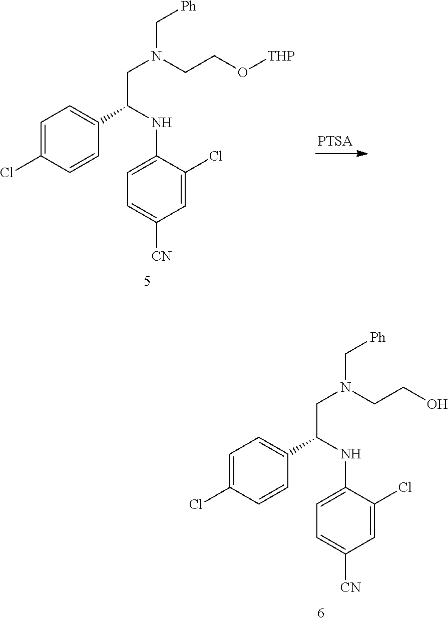

### Step 6

To 5 (178 g, 0.34 mol) in MeOH (1.2 L) at room temperature was added p-toluenesulfonic acid monohydrate (80.7 g, 0.425 mol). Stirred overnight at room temperature. Concentrated in vacuo. Took up into EtOAc (8 L) and washed with 3N NaOH (3×1 L), water, and brine. Dried (MgSO₄), filtered, and concentrated in vacuo. Purified by silica gel chromatography (10%, 15%, 20%, 25% EtOAc/hex) to provide 6 (143 g, 0.33 mol).

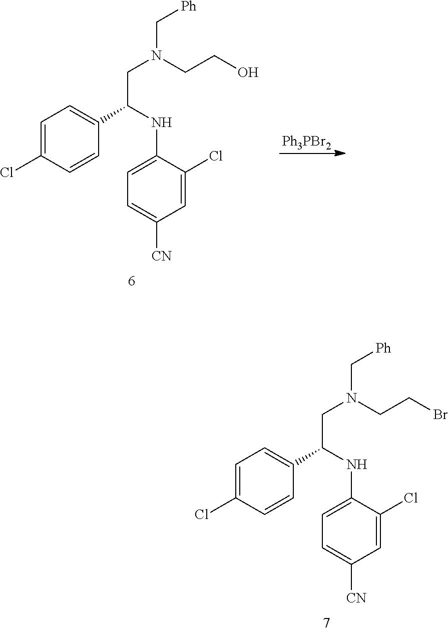

### Step 7

To 6 (140 g, 0.32 mol) in CH₂Cl₂ (1.2 L) at 0° C. was added TEA (110 mL, 0.8 mol) followed by Ph₃PBr₂ (201 g, 0.48 mol). Stirred for 1 h at 0° C. Added CH₂Cl₂ and washed with saturated NaHCO₃, water, and brine. Dried (MgSO₄), filtered, and concentrated in vacuo. Took the residue up into EtOAc (0.3 L) and ether (1.2 L). Filtered and concentrated the filtrate to dryness. Purified by silica gel chromatography (5%, 10%, 13%, 15% EtOAc/hex) to provide 7 (131 g, 0.26 mol).

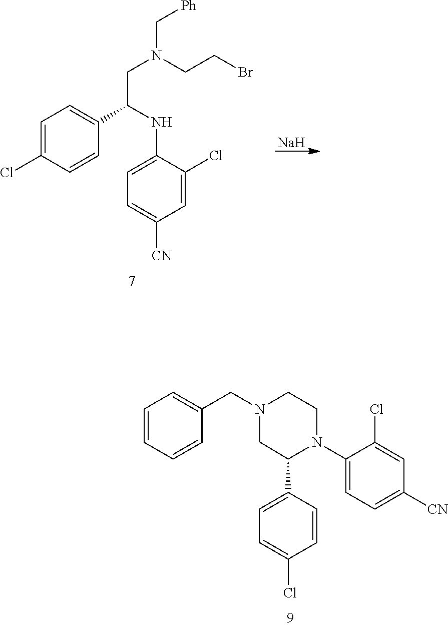

### Step 8

To 7 (129 g, 0.26 mol) in THF (1.5 L) at room temperature was added NaH (60% dispersion in mineral oil, 12.5 g, 0.31 mol) in one portion. Heated the mixture to reflux for 3.5 h. TLC showed slow conversion-added an additional 0.5 equiv of NaH. Stirred at reflux overnight. Cooled to 0° C. and added water (20 mL) slowly. Diluted with EtOAc (8 L) and washed with water and brine. Dried (MgSO₄), filtered, and concentrated in vacuo to provide 9 which was carried on without any further purification.

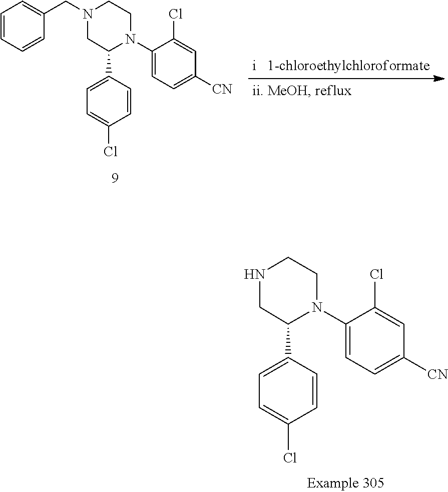 [SMILES: N#Cc1ccc(N2CCN(Cc3ccccc3)C[C@H]2c2ccc(Cl)cc2)c(Cl)c1 ; N#Cc1ccc(N2CCNC[C@H]2c2ccc(Cl)cc2)c(Cl)c1]

### Step 9

To 9 (110 g, —0.26 mol) in dichloromethane (0.9 L) at 0° C. was added 1-chloroethylchloroformate (44.2 mL, 0.41 mol) over 30 minutes. Stirred at 0° C. for 15 minutes and then warmed to reflux. Stirred at reflux for 1 h and then removed the solvent in vacuo. Added MeOH (0.5 L, anhydrous). Warmed to reflux and stirred for 1 h. Removed solvent in vacuo. Took up into water and washed with ether. Filtered off insoluble solid. Added solid NaHCO₃ (80 g) and extracted with CH₂Cl₂. Combined DCM layers and washed with water and brine. Dried (MgSO₄), filtered, and concentrated in vacuo. Purified by silica gel chromatography (2%, 4%, 6% MeOH/CH₂Cl₂) to provide Example 305 (73 g, 0.22 mol).

### Preparation of Example 803

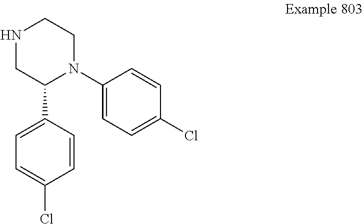 [SMILES: Clc1ccc([C@@H]2CNCCN2c2ccc(Cl)cc2)cc1]

Example 803 was prepared in the same manner as Example 310 except that that 4-chloroaniline was used instead of 4-amino-3-chlorobenzonitrile in step 5.

### Preparation of Example 804

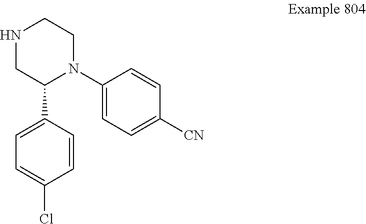 [SMILES: N#Cc1ccc(N2CCNC[C@H]2c2ccc(Cl)cc2)cc1]

Example 804 was prepared in the same manner as Example 310 except that that 4-cyanoaniline was used instead of 4-amino-3-chlorobenzonitrile in step 5.

### Preparation of Example 806

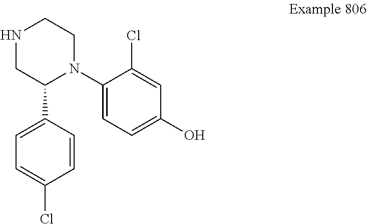 [SMILES: Oc1ccc(N2CCNC[C@H]2c2ccc(Cl)cc2)c(Cl)c1]

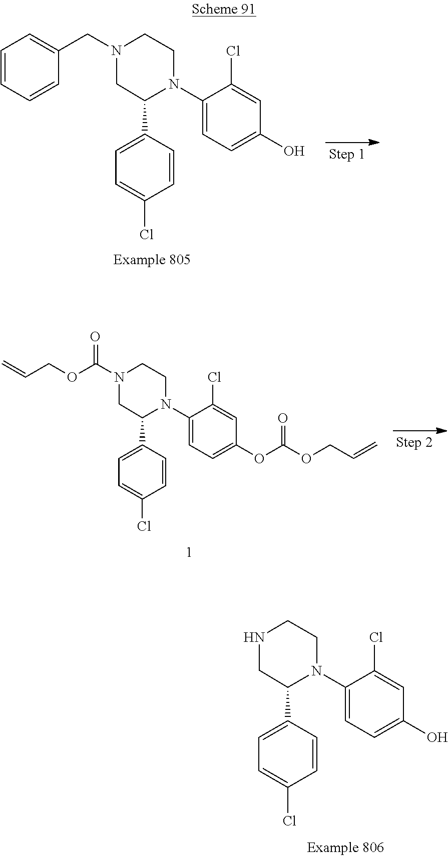 [SMILES: Oc1ccc(N2CCN(Cc3ccccc3)C[C@H]2c2ccc(Cl)cc2)c(Cl)c1 ; C=CCOC(=O)Oc1ccc(N2CCN(C(=O)OCC=C)C[C@H]2c2ccc(Cl)cc2)c(Cl)c1 ; Oc1ccc(N2CCNC[C@H]2c2ccc(Cl)cc2)c(Cl)c1]

### Step 1

To the N-benzyl piperazine Example 805 (2.0 g, 4.85 mmol) [prepared in a manner similar to piperazine 9 of Scheme 90 except that 4-amino-3-chlorophenol was used instead of 4-amino-3-chlorobenzonitrile in step 5] in dichloroethane (16 mL) at room temperature was added diisopropylethyl amine (1.3 mL, 7.27 mmol) followed by allyl chloroformate (1.2 mL, 10.9 mmol). The reaction was warmed to reflux and stirred for 18 h. The reaction was cooled to room temperature and diluted with dichloromethane. The mixture was washed with saturated NaHCO₃, water, and brine. The organic layer was dried (MgSO₄), filtered, and concentrated in vacuo to provide an oil, The product was purified by silica gel chromatography (0-25% EtOAc/hex over 40 minutes) to provide 1 (1.37 g, 2.8 mmol).

### Step 2

To 1 (1.37 g, 2.8 mmol) in acetonitrile (10 mL) and water (10 mL) was added diethylamine (6 mL), trisodiumtriphenylphosphine-3,3′,3″-trisulfonate (0.06 g, 0.04 mmol), and palladium(II)acetate (0.04 g, 0.02 mmol). The reaction was stirred at room temperature for 18 h. The reaction was then concentrated in vacuo leaving ˜10 mL water. EtOAc was added and the mixture stirred vigorously. The mixture was then extracted with EtOAc. The organics were combined and washed with water and brine. The organic layer was dried (MgSO₄), filtered, and concentrated in vacuo. The residue was purified by silica gel chromatography (5-10% MeOH/DCM over 30 minutes) to provide the piperazine Example 806 (0.77 g, 2.26 mmol).

### Preparation of Examples 807 and 808

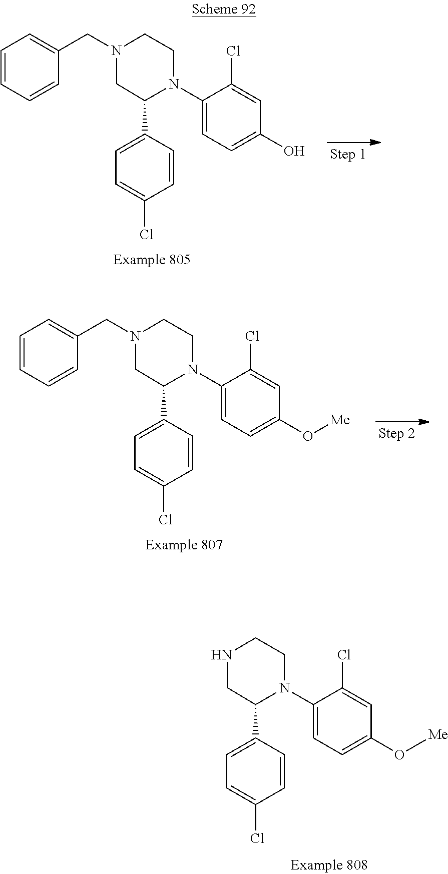 [SMILES: Oc1ccc(N2CCN(Cc3ccccc3)C[C@H]2c2ccc(Cl)cc2)c(Cl)c1 ; COc1ccc(N2CCN(Cc3ccccc3)C[C@H]2c2ccc(Cl)cc2)c(Cl)c1 ; COc1ccc(N2CCNC[C@H]2c2ccc(Cl)cc2)c(Cl)c1]

### Step 1

To Example 805 (0.22 g, 0.52 mmol) in DMSO (1.5 mL) was added potassium tert-butoxide (0.06 g, 0.52 mmol). The reaction was stirred at room temperature for 1 h and methyl sulfate was added (0.05 mL, 0.55 mmol). After 15 minutes, water was added to the reaction. The mixture was extracted with EtOAc. The organic layer was washed with water and brine, dried (MgSO₄), filtered, and concentrated in vacuo. The residue was purified by silica gel chromatography (0-30% EtOAc/hex over 30 minutes) to provide Example 807 (0.18 mg, 0.40 mmol).

### Step 2

Used the conditions found in step 9 of Scheme 90 to afford Example 808.

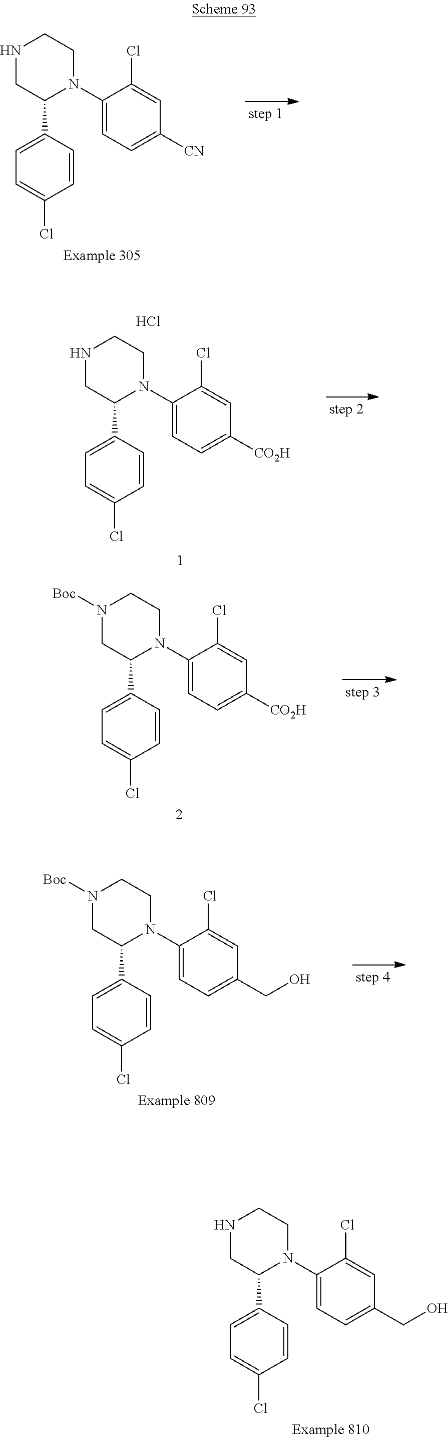 [SMILES: N#Cc1ccc(N2CCNC[C@H]2c2ccc(Cl)cc2)c(Cl)c1 ; Cl ; O=C(O)c1ccc(N2CCNC[C@H]2c2ccc(Cl)cc2)c(Cl)c1 ; CC(C)(C)OC(=O)N1CCN(c2ccc(C(=O)O)cc2Cl)[C@H](c2ccc(Cl)cc2)C1 ; CC(C)(C)OC(=O)N1CCN(c2ccc(CO)cc2Cl)[C@H](c2ccc(Cl)cc2)C1 ; OCc1ccc(N2CCNC[C@H]2c2ccc(Cl)cc2)c(Cl)c1]

### Step 1

To Example 305 (1.50 g, 4.52 mmol) in ethanol (5 mL) was added 3N NaOH (10 mL). Warmed the reaction to 90° C. and stirred for 18 h. Cooled to room temperature and removed the ethanol in vacuo. Added conc. HCl (3 mL) and a solid gummed out onto the stir bar. Cooled to 0° C. and decanted off liquid leaving behind a gum which was carried on directly.

### Step 2

To the gum prepared in step 1 was added CH₂Cl₂ (15 mL) and TEA (1.52 mL, 11.3 mmol). Stirred the mixture for ˜20 minutes at room temperature to allow time for the material to go into solution. Added (Boc)₂O (1.23 g, 5.65 mmol) in one portion and stirred overnight at room temperature. Diluted the reaction with DCM and washed with 1N HCl, water, and brine. Dried (MgSO₄) the organic layer, filtered, and concentrated in vacuo to provide an oil that was carried on directly.

### Step 3

To the material prepared in Step 2 was added THF (15 mL). The mixture was cooled to 0° C. and BH₃ (1.0 M in THF, 6.7 mL, 6.7 mmol) was added. Stirred for 16 h allowing the cold bath to expire. Added aqueous LiOH (2N) [Gas Evolution]. Stirred vigorously for 45 minutes. The mixture was then extracted with EtOAc. The organics were combined and washed with water and brine. Dried (MgSO₄) the organic layer, filtered, and concentrated in vacuo to provide and oil. The material was purified by silica gel chromatography (0-40% EtOAc/hex over 40 minutes) to provide Example 809 (1.12 g, 57% over 3 steps).

### Step 4

To Example 809 (0.39 g, 0.90 mmol) in methylene chloride (3 mL) was added 2N HCl/ether (2 mL). The reaction was stirred at room temperature for 18 h and then concentrated in vacuo. The residue was taken up into methylene chloride and washed with 1N NaOH, water, and brine. The organic layer was dried (MgSO₄), filtered, and concentrated in vacuo to provide Example 810 (0.24 g, 0.72 mmol).

The following epoxides were prepared from the corresponding α-bromoketone using a procedure similar to that described in Scheme 28.

TABLE XXIVa

| En- | | |
|---|---|---|
| try | α-bromoketone | Epoxide |
| 1 | 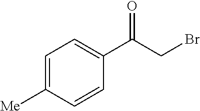 [SMILES: Cc1ccc(C(=O)CBr)cc1] | 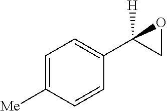 |
| 2 | 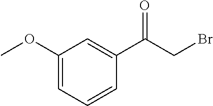 | 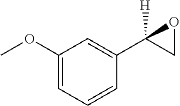 |
| 3 | 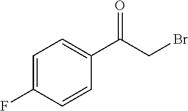 [SMILES: O=C(CBr)c1ccc(F)cc1] | 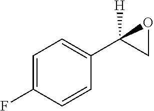 |
| 4 | 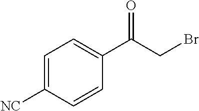 [SMILES: N#Cc1ccc(C(=O)CBr)cc1] | 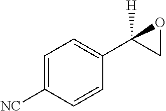 [SMILES: N#Cc1ccc([C@@H]2CO2)cc1] |

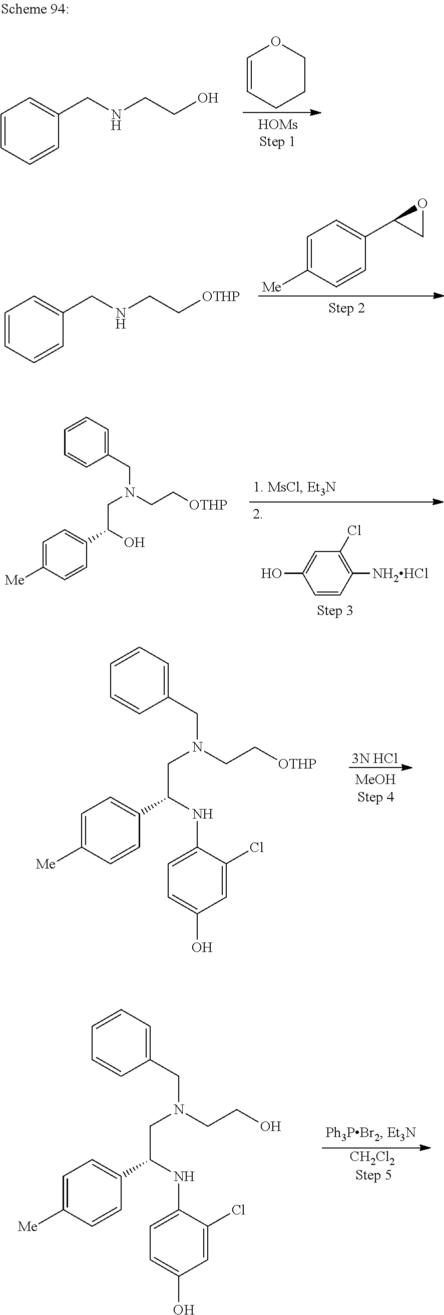

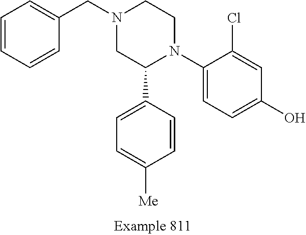 [SMILES: Cc1ccc([C@@H]2CN(Cc3ccccc3)CCN2c2ccc(O)cc2Cl)cc1]

### Step 1:

At room temperature, 2-(benzylamino)ethanol (18.8 mL, 132.3 mmol, 1 eq) was added dropwise to a solution of methanesulfonic acid (9 mL, 139.4 mmol, 1.05 eq) in dichloromethane (325 mL). After stirring for 5 min., 3,4-dihydro-2H-pyran (21 mL, 230.2 mmol, 1.7 eq) was added dropwise to the reaction mixture and the resulting solution was stirred 2 h at room temperature. The reaction mixture was then poured into a stirred solution of 10% aqueous K₂CO₃ (400 mL). After stirring for 10 min., the organic layer was dried over anhydrous Na2SO4, filtered and evaporated to afford an orange oil which was subjected to short-path distillation (0.5-1 mmHg, 132° C.) to provide the THP protected alcohol (20 g, 77% yield) as a clear, colorless oil.

### Step 2:

The styrene oxide (3.35 g, 25.0 mmol, 1 eq) and the THP protected amino alcohol from Step 1 (5.87 g, 25.0 mmol, 1 eq.) were combined neat in a sealed reaction vessel under a nitrogen atmosphere. The vessel was then sealed and heated with stirring at 100° C. for 16 h. After cooling to room temperature, the resulting residue was chromatographed (SiO₂, gradient elution, 0% to 75% EtOAc in hexanes) to afford the desired product (1.86 g, 20% yield) as an oil.

### Step 3:

The product from Step 2 (1.86 g, 5.03 mmol, 1 eq) was dissolved in 1,2-dichloroethane (20 mL) and the resulting solution was cooled to 0° C. Triethylamine (2.5 mL, 17.6 mmol, 3.5 eq) was added to the solution, followed by dropwise addition of methanesulfonyl chloride (0.47 mL, 6.04 mmol, 1.2 eq). After stirring the resulting mixture for 1 h at 0° C. and 1 h at room temperature, the hydrochloride salt of 4-amino-3-chlorophenol (1.13 g, 6.29 mmol, 1.25 eq) was added, and the reaction was heated at 86° C. for 16 h. The reaction was then cooled to room temperature and partitioned between CH₂Cl₂ and saturated aqueous NaHCO₃. The organic layer was saved and the aqueous layer was extracted with CH₂Cl₂. The combined organic layers were washed with brine, dried over anhydrous MgSO₄, filtered, and evaporated to afford a dark red oil which was purified via silica gel chromatography (gradient elution, 20% to 60% EtOAc in hexanes) to afford the desired product (1.03 g, 41% yield) as an oil.

### Step 4:

The product from Step 3 (1.03 g, 2.08 mmol) was dissolved in methanol (25 mL). At room temperature, with stirring, 3N aqueous HCl (9 mL) was added, and the resulting mixture was stirred for 3 h. The reaction mixture was then poured into a stirred mixture of EtOAc (75 mL), water (50 mL) and NaHCO₃ (1.5 g). After stirring for 30 min., the aqueous layer was removed and extracted again with EtOAc. The organic extracts were combined and evaporated to afford a pale orange oil which was subjected to silica gel chromatography (gradient elution, 20% to 80% EtOAc in hexanes) to afford the desired product (496 mg, 58% yield) as a clear viscous oil.

### Step 5:

The product from Step 4 (490 mg, 1.19 mmol, 1 eq) and triethylamine (0.42 mL, 2.98 mmol, 2.5 eq) were dissolved in CH₂Cl₂ (5 mL). While stirring at room temperature, triphenylphosphine dibromide (755 mg, 1.79 mmol, 1.5 eq) was added, and the reaction was stirred for 4 h at room temperature. The reaction mixture was concentrated, and the resulting residue purified via silica gel chromatography (gradient elution, 20% to 80% EtOAc in hexanes) to afford Example 811 (330 mg, 71% yield) as a clear, colorless film.

Using the appropriate styrene oxide and aniline, the following compounds were prepared in a method similar that of Scheme 94.

TABLE XXIVb

| Example | Styrene | | |
|---|---|---|---|
| # | oxide | aniline | example structure |
| 812a | 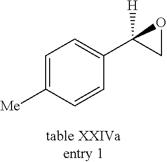 | 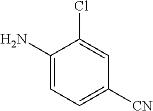 | 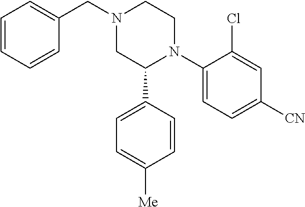 [SMILES: Cc1ccc([C@@H]2CN(Cc3ccccc3)CCN2c2ccc(C#N)cc2Cl)cc1] |
| 812b | 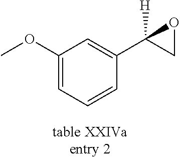 | 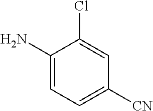 | 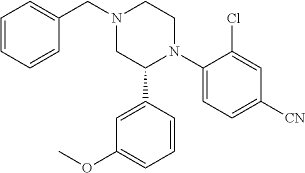 [SMILES: COc1cccc([C@@H]2CN(Cc3ccccc3)CCN2c2ccc(C#N)cc2Cl)c1] |

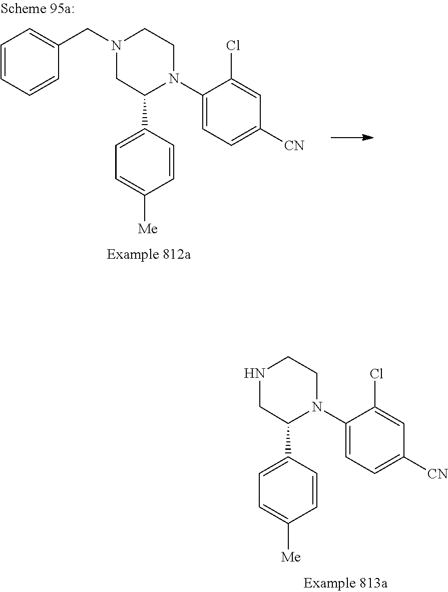 [SMILES: Cc1ccc([C@@H]2CN(Cc3ccccc3)CCN2c2ccc(C#N)cc2Cl)cc1 ; Cc1ccc([C@@H]2CNCCN2c2ccc(C#N)cc2Cl)cc1]

To Example 812a (4.55 g, 11.32 mmol, 1 eq) in DCM (40 mL) was added 1-chloroethylchloroformate (2.2 mL, 20.38 mmol, 1.8 eq). The reaction mixture was stirred at reflux for 2 h, and then was stirred 72 h at room temperature. The reaction was concentrated in vacuo, and the resulting residue was taken up in MeOH (40 mL) and warmed to reflux. After stirring for 1.5 h, the reaction mixture was concentrated in vacuo. The residue was taken up into DCM and washed with saturated NaHCO₃. The organic layer was adsorbed onto silica gel and then subjected to silica gel chromatography (gradient elution, 0-15% MeOH in DCM) to provide Example 813a (3.49 g, 99% yield) as a yellow foam.

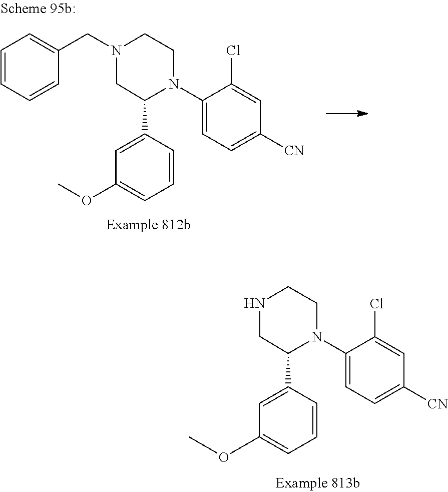 [SMILES: COc1cccc([C@@H]2CN(Cc3ccccc3)CCN2c2ccc(C#N)cc2Cl)c1 ; COc1cccc([C@@H]2CNCCN2c2ccc(C#N)cc2Cl)c1]

Example 813b was prepared from Example 812b using a method similar to that described in Scheme 95a.

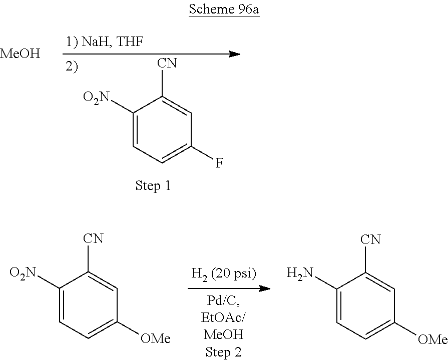 [SMILES: "MeOH" ; N#Cc1cc(F)ccc1[N+](=O)[O-] ; COc1ccc([N+](=O)[O-])c(C#N)c1 ; COc1ccc(N)c(C#N)c1]

### Step 1:

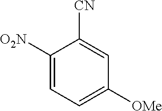 [SMILES: COc1ccc([N+](=O)[O-])c(C#N)c1]

To a slurry of NaH (60% in oil)(7.2 g, 181 mmol) in anhydrous THF (300 mL) at 0° C. was added dropwise anhydrous MeOH (5.8 g, 181 mmol). Once the addition was complete, the resultant mixture was stirred at 0° C. for an additional 20 min. The slurry was added slowly via cannula to a solution of 5-fluoro-2-benzonitrile (25 g, 150 mmol) in THF (100 mL). The green solution was allowed to slowly warm to RT and stir overnight. Water was slowly added and the aqueous layer was extracted with EtOAc (3×). The combined organic layers were washed with brine, dried over Na₂SO₄, filtered and concentrated. The crude product was purified via flash chromatography (SiO₂: gradient elution, 100:0 to 60:40 hexanes:EtOAc) to afford the nitro compound (23.2 g, 87%) as a light yellow solid.

### Step 2:

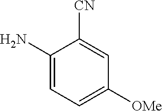 [SMILES: COc1ccc(N)c(C#N)c1]

A solution of the nitro compound from Step 1 (6.5 g, 36 mmol) in 4:1 EtOAc:MeOH (150 mL) in a pressure vessel was degassed with by bubbling N₂ through the solution for 10 min. To this solution was added 10% Pd/C (300 mg). The vessel was sealed and pressurized with H₂ to 25 psi. The vessel was then shaken at RT for 20 min. Once the reaction was complete, the vessel was purged with N₂. The mixture was filtered through Celite and concentrated. The crude product was purified via flash chromatography (SiO₂: gradient elution, 100:0 to 60:40 hexanes:EtOAc) to afford the aniline (6.4 g, 100%) as a light yellow solid.

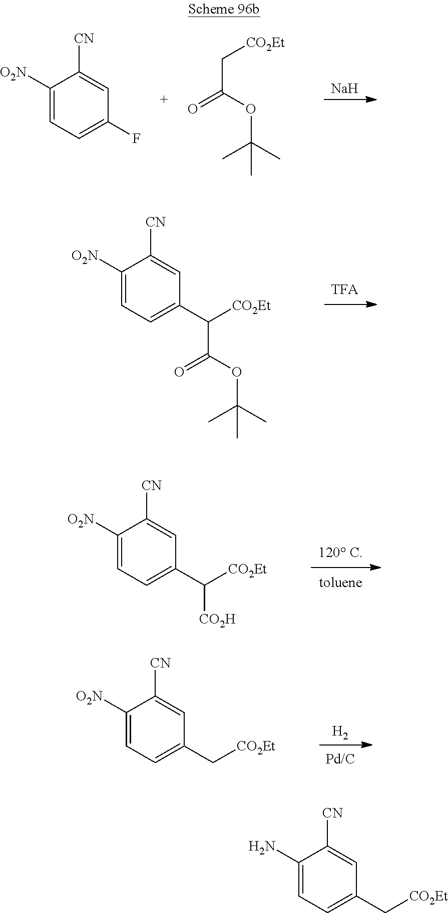

### Step 1

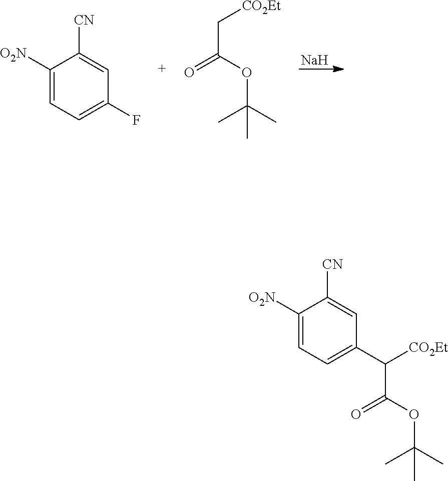

The malonate (22.7 g, 120 mmol) was taken up in DMF (100 ml) at 0° C. Sodium hydride (4.8 g of a 60 wt % dispersion in oil) was added in portions to the solution at 0° C. (gas evolution). The solution was allowed to warm to 25° C. and stirred at that temperature for 30 minutes. The solution was cooled to 0° C., and the aryl fluoride (10 g, 60 mmol) was added to the solution at 0° C. The solution was allowed to warm to 25° C. and stir at that temperature for 18 h. The solution was partitioned between EtOAc and water. The aqueous layer was extracted with EtOAc. The combined organic layers were washed with brine and dried (MgSO₄). Filtration and concentration gave an orange solid. The solid was taken up in Et₂O and triturated with hexanes which provided 15.6 g (78%) of the tert-butyl ester as a yellow solid.

### Step 2

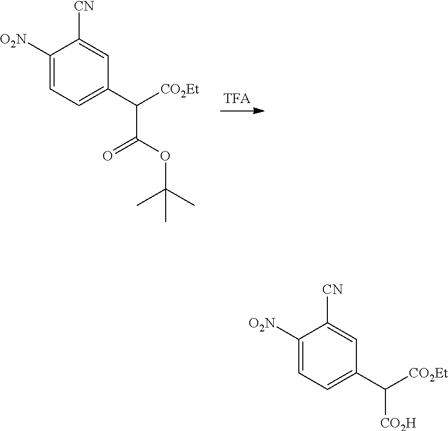 [SMILES: CCOC(=O)C(C(=O)OC(C)(C)C)c1ccc([N+](=O)[O-])c(C#N)c1 ; CCOC(=O)C(C(=O)O)c1ccc([N+](=O)[O-])c(C#N)c1]

The tert-butyl ester (15.6 g, 46.7 mmol) was taken up in DCM (150 ml), and TFA (50 ml) was added. The solution was stirred at 25° C. for 18 h. The solution was concentrated. The acid was used without further purification in the next step.

### Step 3

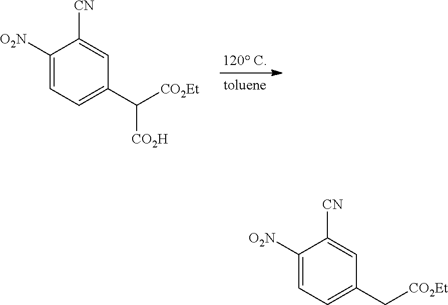 [SMILES: CCOC(=O)C(C(=O)O)c1ccc([N+](=O)[O-])c(C#N)c1 ; CCOC(=O)Cc1ccc([N+](=O)[O-])c(C#N)c1]

The acid (˜46.7 mmol) was taken up in toluene (100 ml), and the resulting solution was heated at 120° C. for 3.5 h. The solution was cooled and concentrated. The residue was triturated with Et₂O/hexanes which provided 8 g (73%) of the ethyl ester as a white solid.

### Step 4

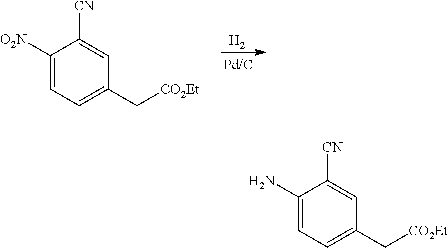 [SMILES: CCOC(=O)Cc1ccc([N+](=O)[O-])c(C#N)c1 ; "H2" ; CCOC(=O)Cc1ccc(N)c(C#N)c1]

The nitro benzene (8.7 g, 37.2 mmol) and 10% Pd/C (400 mg) were taken up in EtOH/EtOAc (1/1, 100 ml) under 10 psi H₂. The mixture was shaken in a Parr apparatus for 20 minutes. The mixture was filtered and concentrated which gave an orange residue. The material was purified via gradient flash chromatography (0-25% EtOAc in hexanes, SiO₂) which provided 7.76 g (Quant.) of the amine as an orange oil.

The following examples were prepared in a similar manner as depicted in Scheme 90 using the appropriate reagents shown in Table XXV.

TABLE XXV

| Epoxide | Aniline | Ex | Structure |
|---|---|---|---|
| 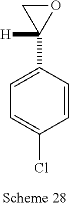 | 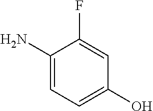 | 814 | 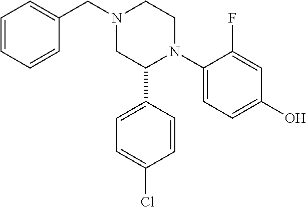 [SMILES: Oc1ccc(N2CCN(Cc3ccccc3)C[C@H]2c2ccc(Cl)cc2)c(F)c1] |
| 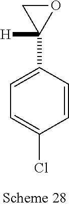 | 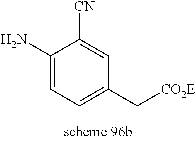 | 815 | 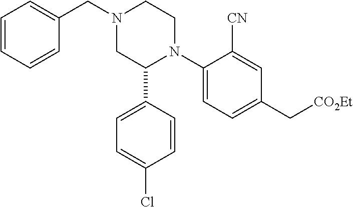 [SMILES: CCOC(=O)Cc1ccc(N2CCN(Cc3ccccc3)C[C@H]2c2ccc(Cl)cc2)c(C#N)c1] |
| 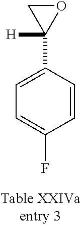 | 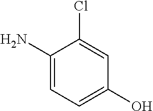 | 816 | 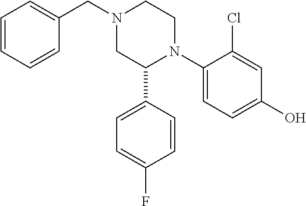 [SMILES: Oc1ccc(N2CCN(Cc3ccccc3)C[C@H]2c2ccc(F)cc2)c(Cl)c1] |
| 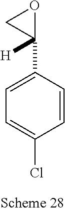 | 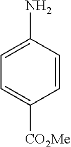 [SMILES: COC(=O)c1ccc(N)cc1] | 817 | 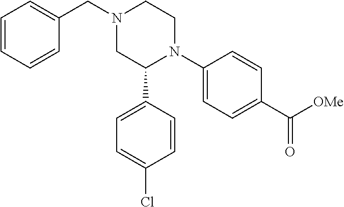 [SMILES: COC(=O)c1ccc(N2CCN(Cc3ccccc3)C[C@H]2c2ccc(Cl)cc2)cc1] |
| 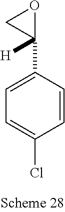 | 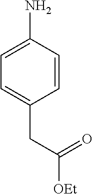 [SMILES: CCOC(=O)Cc1ccc(N)cc1] | 818 | 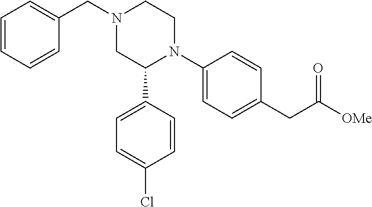 [SMILES: COC(=O)Cc1ccc(N2CCN(Cc3ccccc3)C[C@H]2c2ccc(Cl)cc2)cc1] |
| 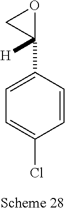 | 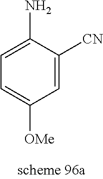 [SMILES: COc1ccc(N)c(C#N)c1] | 819 | 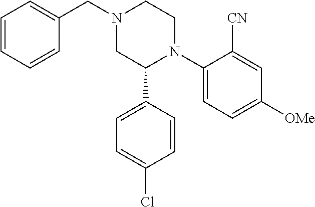 [SMILES: COc1ccc(N2CCN(Cc3ccccc3)C[C@H]2c2ccc(Cl)cc2)c(C#N)c1] |
| 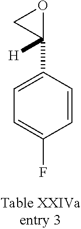 | 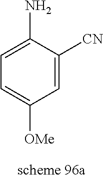 [SMILES: COc1ccc(N)c(C#N)c1] | 820 | 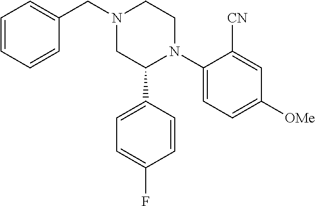 [SMILES: COc1ccc(N2CCN(Cc3ccccc3)C[C@H]2c2ccc(F)cc2)c(C#N)c1] |
| 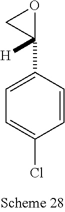 | 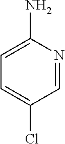 | 820a | 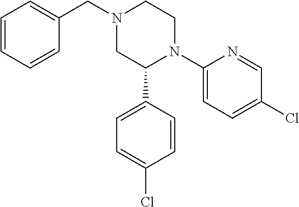 [SMILES: Clc1ccc([C@@H]2CN(Cc3ccccc3)CCN2c2ccc(Cl)cn2)cc1] |
| 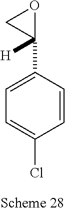 | 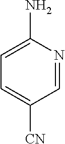 | 820b | 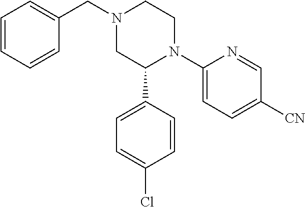 [SMILES: N#Cc1ccc(N2CCN(Cc3ccccc3)C[C@H]2c2ccc(Cl)cc2)nc1] |
| 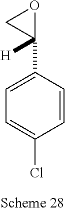 | 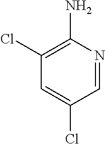 | 820c | 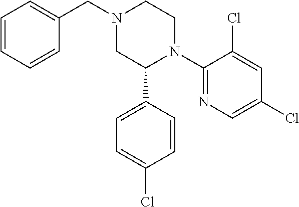 [SMILES: Clc1ccc([C@@H]2CN(Cc3ccccc3)CCN2c2ncc(Cl)cc2Cl)cc1] |
| 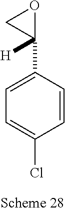 | 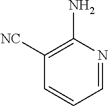 | 820d | 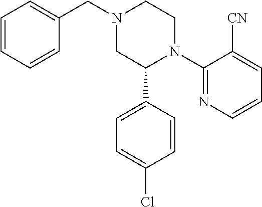 [SMILES: N#Cc1cccnc1N1CCN(Cc2ccccc2)C[C@H]1c1ccc(Cl)cc1] |
| 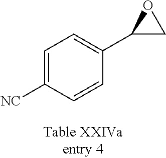 [SMILES: N#Cc1ccc([C@@H]2CO2)cc1] | 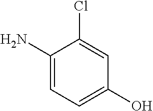 | 820e | 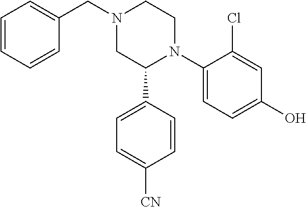 [SMILES: N#Cc1ccc([C@@H]2CN(Cc3ccccc3)CCN2c2ccc(O)cc2Cl)cc1] |

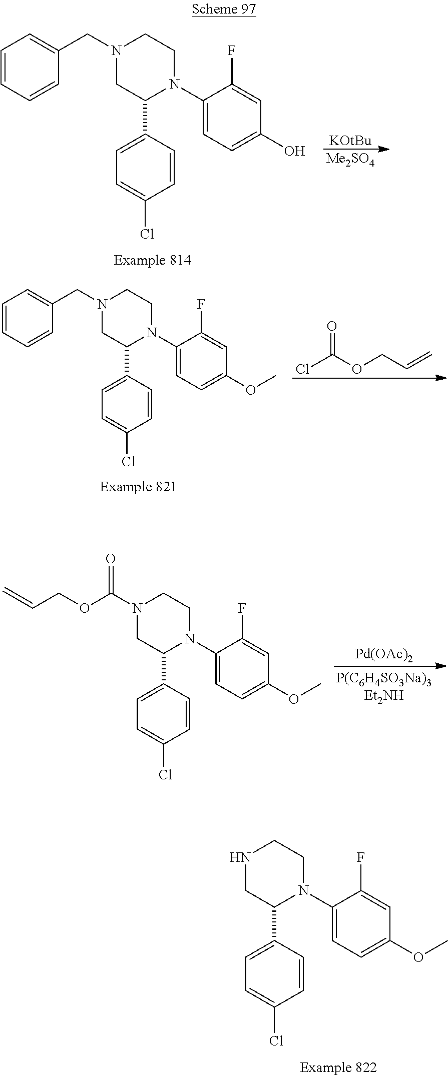

### Step 1

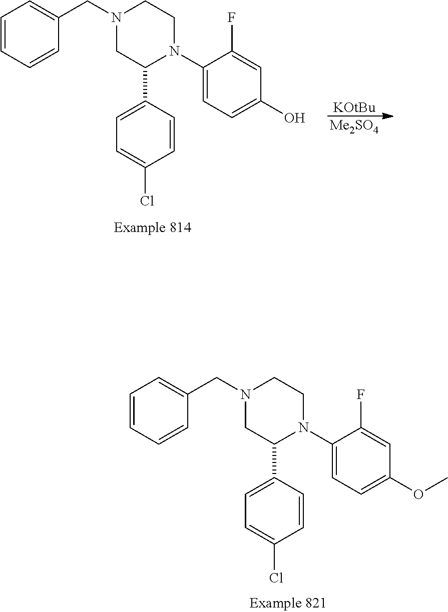 [SMILES: "Me2SO4" ; Oc1ccc(N2CCN(Cc3ccccc3)C[C@H]2c2ccc(Cl)cc2)c(F)c1 ; COc1ccc(N2CCN(Cc3ccccc3)C[C@H]2c2ccc(Cl)cc2)c(F)c1 ; "KOtBu"]

Example 814 (2.46 g, 6.2 mmol) and KOtBu (696 mg) were taken up in DMSO (15 ml). After stirring at 25° C. for 1 h, dimethyl sulfate (829 mg) was added, and the resulting solution was stirred at 25° C. for 30 minutes. The solution was partitioned between EtOAc and water. The aqueous layer was extracted with EtOAc. The combined organic layers were washed with brine and dried (MgSO₄). Filtration and concentration gave a yellow oil. The residue was purified via gradient flash chromatography (0-20% EtOAc in hexanes, SiO₂) which provided 1.62 g (63%) of Ex. 821 as a colorless foam.

### Step 2

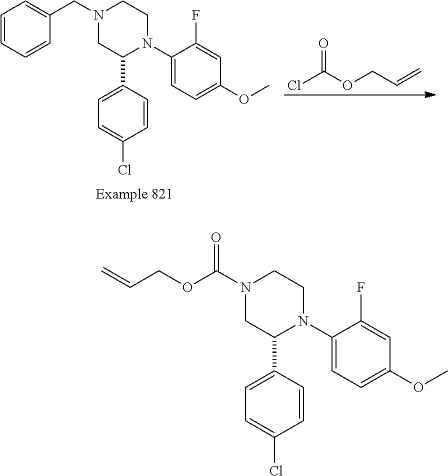

Example 821 (1.6 g, 3.95 mmol), iPr₂NEt (764 mg), and allyl chloroformate (570 mg) were taken up in DCE (35 ml), and the solution was heated at 85° C. for 18 h. The solution was diluted with DCM and washed with sat. NaHCO_{3(aq.)}. The aqueous layer was extracted with DCM. The combined organic layers were dried (MgSO₄), filtered, and concentrated. The residue was purified via flash chromatography (1/1 DCM/hexanes, SiO₂) which provided 1.58 g (99%) of the carbamate as a colorless oil.

### Step 3

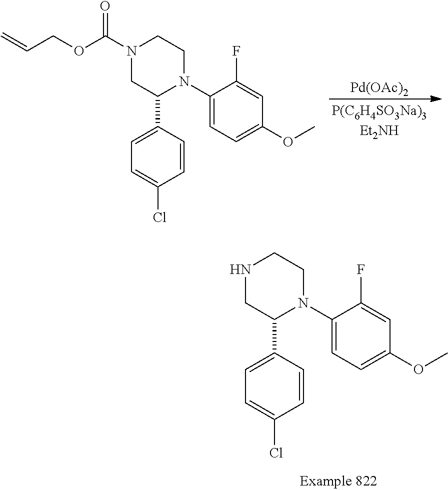 [SMILES: C=CCOC(=O)N1CCN(c2ccc(OC)cc2F)[C@H](c2ccc(Cl)cc2)C1 ; COc1ccc(N2CCNC[C@H]2c2ccc(Cl)cc2)c(F)c1 ; "Pd(OAc)2"]

The carbamate (1.58 g, 3.9 mmol), phosphine (44 mg), and Et₂NH (5.7 g) were taken up in CH₃CN/water (1/1, 20 ml). Palladiium (II) acetate (9 mg) was added, and the solution was stirred at 25° C. for 18 h. The solution was concentrated, and the residue was partitioned between EtOAc and 1 N NaOH_{(aq.)}. The aqueous layer was extracted with EtOAc. The combined organic layers were washed with brine and dried. Filtration and concentration provided a yellow oil. The residue was purified via gradient flash chromatography (0-25% MeOH in EtOAc, SiO₂) which gave 540 mg (69%) Example 822 as a yellow foam.

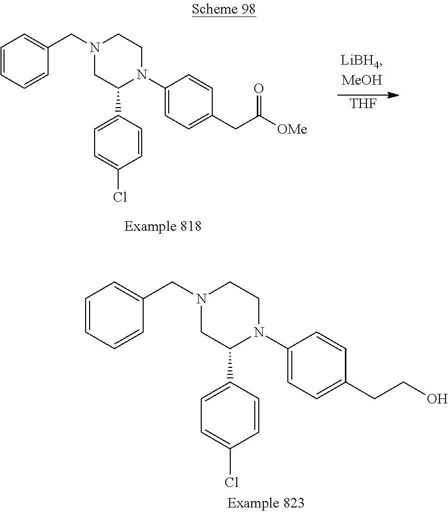 [SMILES: COC(=O)Cc1ccc(N2CCN(Cc3ccccc3)C[C@H]2c2ccc(Cl)cc2)cc1 ; OCCc1ccc(N2CCN(Cc3ccccc3)C[C@H]2c2ccc(Cl)cc2)cc1 ; "THF"]

To a solution of the ester (4.20 g, 9.7 mmol) in THF (50 mL) was added MeOH (330 mg, 9.7 mmol) and LiBH₄ (315 mg, 14.5 mmol). The mixture was heated to reflux for 3.5 hours. The mixture was allowed to cool to room temperature. A slight excess 1 M HCl (aq.) was added to quench the reaction. After a short time, excess 1 M NaOH (aq.) was added and the mixture was extracted with EtOAc (3×). The combined organic layers were washed with brine, dried over Na₂SO₄, filtered and concentrated. The crude product was purified via flash chromatography (SiO₂: gradient elution 100:0 to 65:35 hexanes:EtOAc) to afford the alcohol (3.7 g) as a white foam.

The following example was prepared using a similar procedure to that described in Scheme 98.

TABLE XXVI

| Entry | Ester | Ex. | Alcohol |
|---|---|---|---|
| 1 | 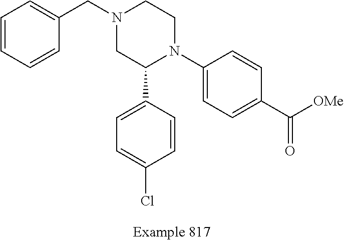 [SMILES: COC(=O)c1ccc(N2CCN(Cc3ccccc3)C[C@H]2c2ccc(Cl)cc2)cc1] | 824 | 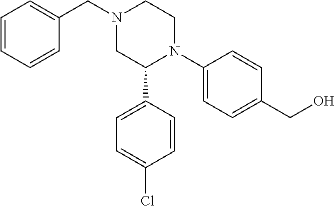 [SMILES: OCc1ccc(N2CCN(Cc3ccccc3)C[C@H]2c2ccc(Cl)cc2)cc1] |

The following examples were prepared using a similar procedure to that described in Scheme 90 Step 9.

TABLE XXVII

| Example | N-Benzyl core | Example | NH core |
|---|---|---|---|
| 824 | 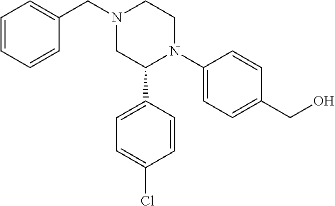 [SMILES: OCc1ccc(N2CCN(Cc3ccccc3)C[C@H]2c2ccc(Cl)cc2)cc1] | 825 | 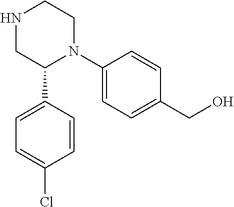 [SMILES: OCc1ccc(N2CCNC[C@H]2c2ccc(Cl)cc2)cc1] |
| 823 | 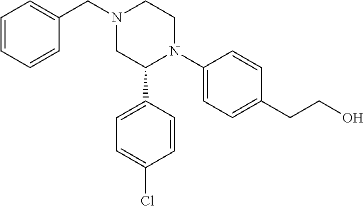 [SMILES: OCCc1ccc(N2CCN(Cc3ccccc3)C[C@H]2c2ccc(Cl)cc2)cc1] | 826 | 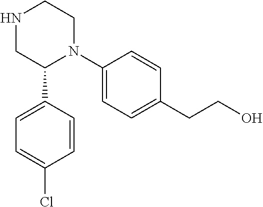 [SMILES: OCCc1ccc(N2CCNC[C@H]2c2ccc(Cl)cc2)cc1] |
| 820a | 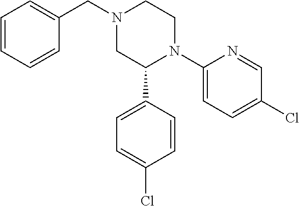 [SMILES: Clc1ccc([C@@H]2CN(Cc3ccccc3)CCN2c2ccc(Cl)cn2)cc1] | 826a | 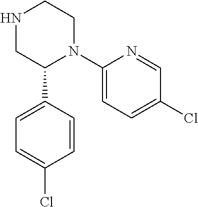 [SMILES: Clc1ccc([C@@H]2CNCCN2c2ccc(Cl)cn2)cc1] |
| 820b | 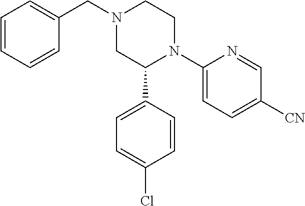 [SMILES: N#Cc1ccc(N2CCN(Cc3ccccc3)C[C@H]2c2ccc(Cl)cc2)nc1] | 826b | 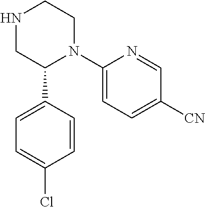 [SMILES: N#Cc1ccc(N2CCNC[C@H]2c2ccc(Cl)cc2)nc1] |
| 820c | 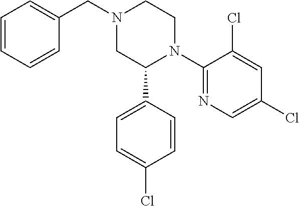 [SMILES: Clc1ccc([C@@H]2CN(Cc3ccccc3)CCN2c2ncc(Cl)cc2Cl)cc1] | 826c | 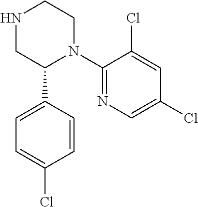 [SMILES: Clc1ccc([C@@H]2CNCCN2c2ncc(Cl)cc2Cl)cc1] |
| 820d | 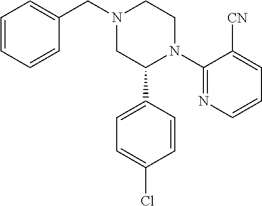 [SMILES: N#Cc1cccnc1N1CCN(Cc2ccccc2)C[C@H]1c1ccc(Cl)cc1] | 826d | 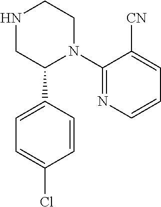 [SMILES: N#Cc1cccnc1N1CCNC[C@H]1c1ccc(Cl)cc1] |
| 820 | 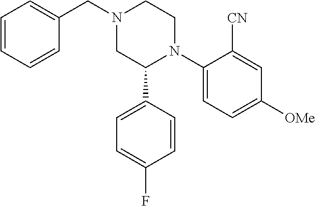 [SMILES: COc1ccc(N2CCN(Cc3ccccc3)C[C@H]2c2ccc(F)cc2)c(C#N)c1] | 826e | 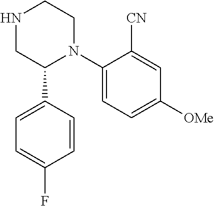 [SMILES: COc1ccc(N2CCNC[C@H]2c2ccc(F)cc2)c(C#N)c1] |

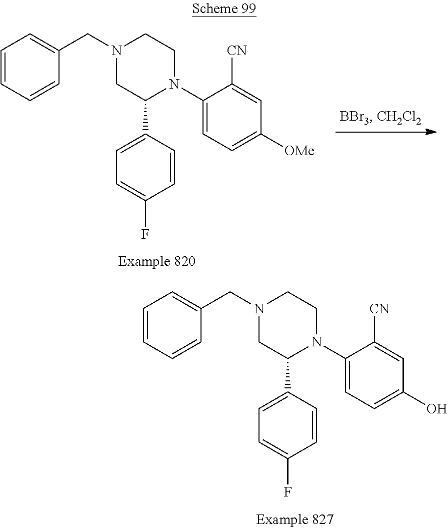 [SMILES: COc1ccc(N2CCN(Cc3ccccc3)C[C@H]2c2ccc(F)cc2)c(C#N)c1 ; N#Cc1cc(O)ccc1N1CCN(Cc2ccccc2)C[C@H]1c1ccc(F)cc1]

To a solution of Example 820 (1.35 g, 3.4 mmol) in CH₂Cl₂ (50 mL) at 0° C. was added BBr₃ (1.26 g, 5.0 mmol). The resultant solution was stirred at 0° C. for 4 hours. Warmed to RT and added additional BBr₃ (1.26 g, 5.0 mmol). The resultant solution was stirred at RT overnight. The solution was poured into sat. NaHCO₃ (aq.). The aqueous layer was extracted with CH₂Cl₂ (3×). The combined organic layers were dried over Na₂SO₄, filtered and concentrated. The crude product was purified via flash chromatography (SiO₂: gradient elution 100:0 to 50:50 hexanes:EtOAc) to afford Example 827 as a tan foam.

### Preparation of Example 828 and Example 829

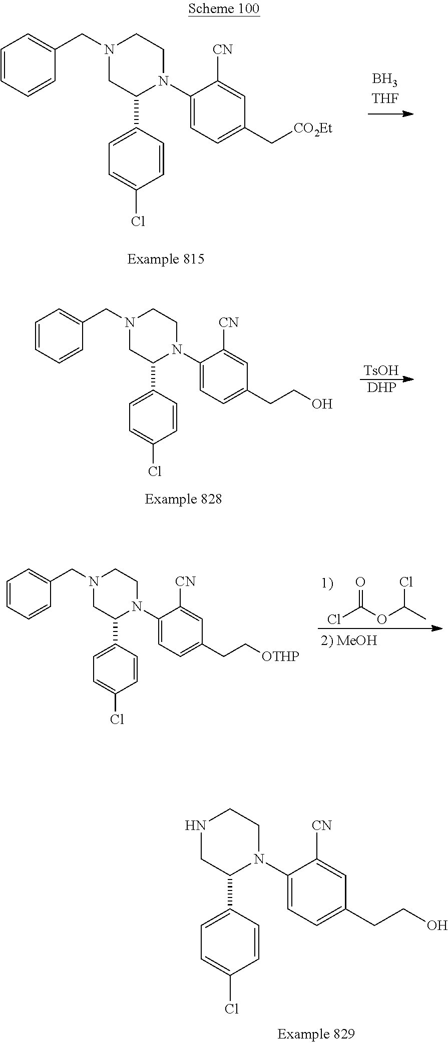

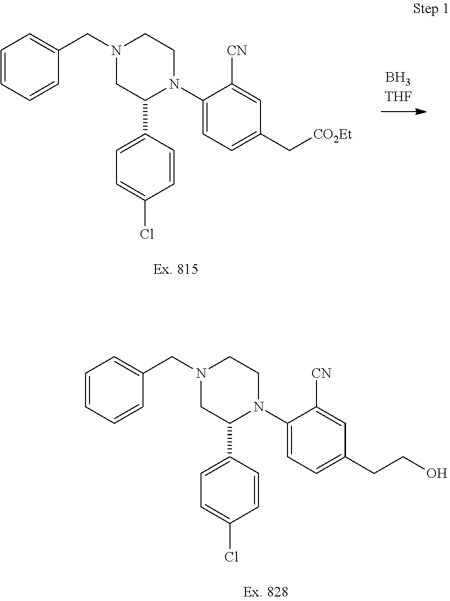

Example 815 (3.5 g, 7.85 mmol) was taken up in THF (30 ml) at 0° C. Borane THF (1.0M in THF, 11.8 ml) was added dropwise to the solution at 0° C. The solution was warmed to 25° C. and stirred at that temperature for 5.5 h. The solution was quenched with 1 M HCl_{(aq.)}. The mixture was stirred at 25° C. for 1.5 h. The solution was concentrated, and the residue was partitioned between water and EtOAc. The mixture was made basic via addition of solid NaHCO₃. The aqueous layer was extracted with EtOAc. The combined organic layers were washed with brine and dried MgOS₄). Filtration and concentration gave a yellow oil. The residue was purified via gradient flash chromatography (0-65% EtOAc in hexanes, SiO₂) which provided 1.04 g (30%) of Ex. 828 as a colorless oil.

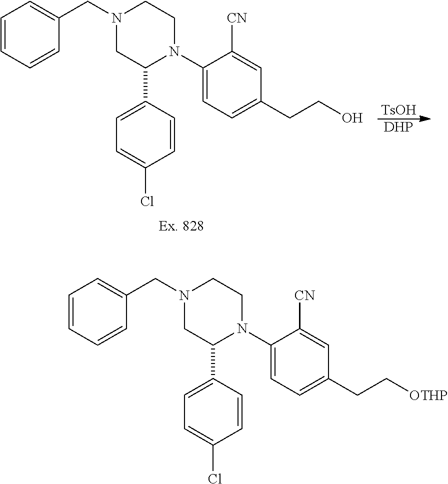

### Step 2

Example 828 (1.04 g, 2.4 mmol), TsOH—H₂O (550 mg), and DHP (1 ml) were taken up in DCM (35 ml) at 25° C. for 18 h. The solution was diluted with DCM and washed with 1 N NaOH_{(aq.)}. The aqueous layer was extracted with DCM. The organic layers were dried (MgSO₄), filtered, and concentrated. The residue was purified via gradient flash chromatography (0-35% EtOAc in hexanes, SiO₂) which provided 1.1 g (88%) of the THP ether.

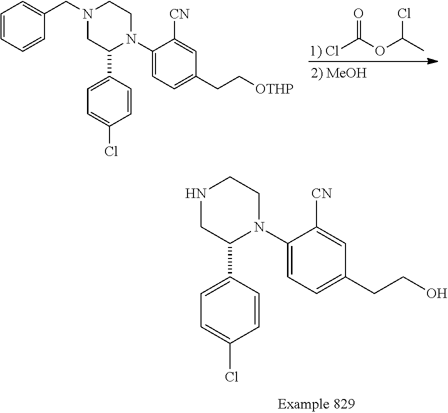

### Step 3

The THP ether was converted to Example 829 in a manner similar to that described in Step 6 of Scheme 90.

### Preparation of Example 830

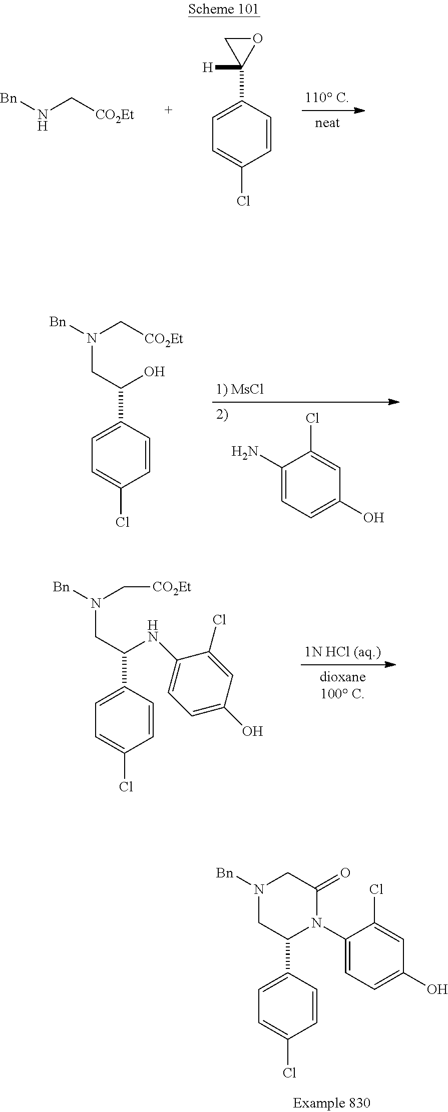

### Step 1

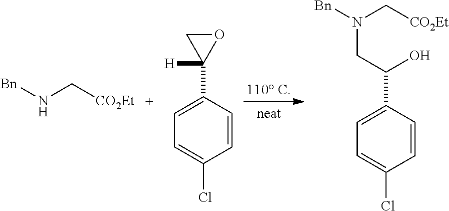

N-Benzyl glycine ethyl ester (6.2 g, 32 mmol) and epoxide (5.0 g, 32 mmol) were heated neat at 110° C. for 18 h. The residue was purified via gradient flash chromatography (0-15% EtOAc in hexanes, SiO₂) which provided 3.4 g (31%) of the alcohol as a colorless oil.

### Step 2

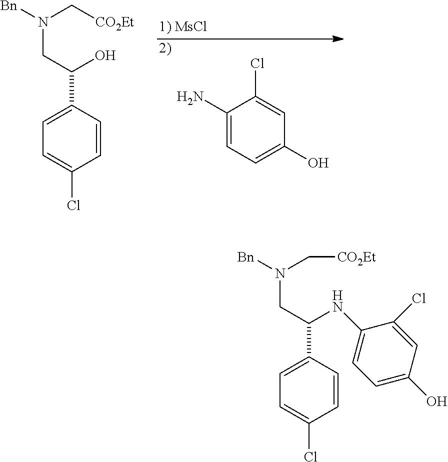

The alcohol (3.4 g, 9.8 mmol) and Et₃N (3.4 ml) were taken up in DCE (65 ml) at 0° C. Methanesulfonyl chloride (0.8 ml, 10 mmol) was added, and the resulting solution was stirred at 0° C. for 15 minutes. The aniline (1.5 g, 10 mmol) was added, and the resulting mixture was heated at 80° C. for 18 h. The solution was diluted with DCM and washed with sat. NaHCO₃ (aq.). The aqueous layer was extracted with DCM. The combined organic layers were dried (MgSO₄), filtered, and concentrated. The residue was purified via gradient flash chromatography (0-20% EtOAc in hexanes, SiO₂) which provided 3.9 g (84%) of the amine as a yellow gum.

### Step 3

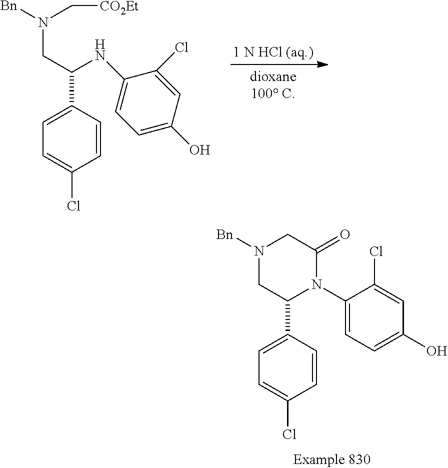

The amine (940 mg, 1.99 mol) was taken up in 1 M HCl (aq.) and dioxane (1/1, 20 ml). The solution was heated at 100° C. for 20 h. The solution was concentrated, and the residue was partitioned between EtOAc and sat. NaHCO₃ (aq.). The aqueous layer was extracted with EtOAc. The combined organic layers were washed with brine and dried (MgSO₄). Filtration and concentration gave a yellow oil. The residue was purified via gradient flash chromatography (0-35% EtOAc in DCM, SiO₂) which provided 628 mg (74%) of Ex. 830 as a colorless foam.

### Preparation of Example 831

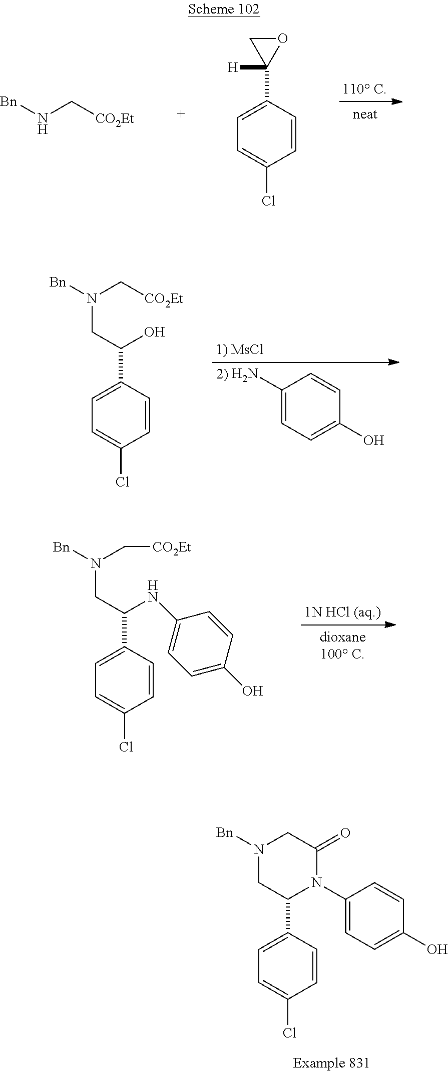

Example 831 was prepared in a manner similar to that depicted in Scheme 101 except 4-hydroxyaniline was used in Step 2 instead of 4-amino-3-chlorophenol.

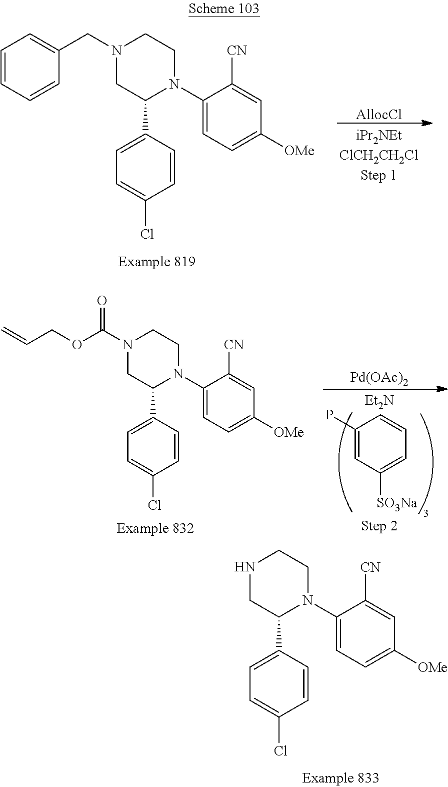

### Step 1:

To a solution of Example 819 (7.64 g, 18.3 mmol) in 1,2-dichloroethane was added diisopropylethylamine (4.73 g, 36.6 mmol) and allyl chloroformate (3.3 g, 27.4 mmol). The resultant solution was heated to reflux with stirring overnight. After that time, additional diisopropylethylamine (2.36 g, 18.3 mmol) and allyl chloroformate (1.6 g, 13.7 mmol) were added and the solution was heated to reflux for an additional 3 hours. The solution was concentrated and the crude residue was purified via flash chromatography (SiO₂: gradient elution 100:0 to 70:30 hexanes:EtOAc) to afford Example 832 (6.85 g).

### Step 2:

To a solution of Example 832 from step 1 (4.0 g, 9.7 mmol) in 1:1 MeCN/water (200 mL) was added diethylamine (14 g, 190 mmol), palladium acetate (25 mg, 0.097 mmol) and tris(3-sulfonatophenyl)phosphine trisodium salt (110 mg, 0.19 mmol). The resulting solution was stirred at RT for 3 hours. The solution was concentrated in vacuo. The residue was taken up in toluene and concentrated in vacuo (3×). The crude residue was purified via flash chromatography [SiO₂: gradient elution 100:0:0 to 96.5:3.5:0.35 CH₂Cl₂: MeOH: conc. NH₄OH (aq.)] to afford Example 833 (2.6 g).

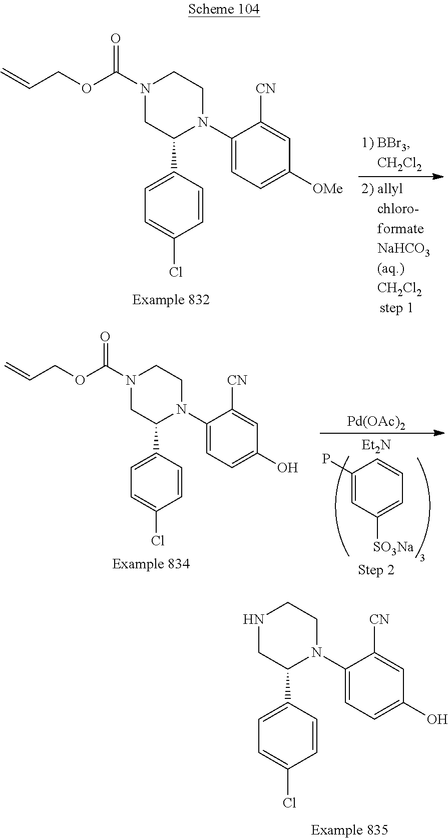

### Step 1:

To a solution of Example 832 (2.3 g, 5.6 mmol) in CH₂Cl₂ (15 mL) at 0° C. was added BBr₃ (3.5 g, 14 mmol). The resulting solution was warmed to RT with stirring overnight. To this solution was added sat. NaHCO₃ (aq.) (20 mL). The mixture was stirred at RT for 1 hour. To this mixture was added allyl chloroformate (1.01 g, 8.4 mmol). The resulting mixture was stirred at RT for 1.5 hours. After that time the aqueous layer was extracted with CH₂Cl₂. The organic layer was dried over Na₂SO₄, filtered and concentrated. The crude product was purified via flash chromatography (SiO₂: gradient elution 100:0 to 70:30 hexanes:EtOAc) to afford Example 834.

### Step 2:

Example 835 was prepared using conditions similar to that described in Scheme 97 step 3, except Example 834 was used.

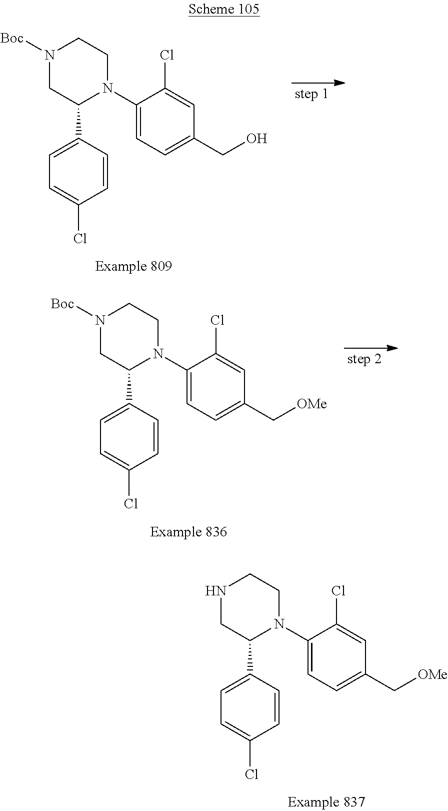 [SMILES: CC(C)(C)OC(=O)N1CCN(c2ccc(CO)cc2Cl)[C@H](c2ccc(Cl)cc2)C1 ; COCc1ccc(N2CCN(C(=O)OC(C)(C)C)C[C@H]2c2ccc(Cl)cc2)c(Cl)c1 ; COCc1ccc(N2CCNC[C@H]2c2ccc(Cl)cc2)c(Cl)c1]

### Step 1

To Example 809 (0.20 g, 0.46 mmol) in THF (1.5 mL) was added NaH (60% dispersion in mineral oil, 28 mg, 0.69 mmol). The mixture was stirred at room temperature for 45 minutes. Cooled to 0 C and added methyl iodide (0.057 mL, 0.92 mmol). Took cold bath away and stirred for 4 h. Added water to the mixture and extracted with EtOAc. Combined organics and washed with water and brine. Dried (MgSO₄), filtered, and concentrated in vacuo. Purified the residue by silica gel chromatography (0-15% EtOAc/hex over 30 minutes) to provide Example 836 (0.14 g, 68%).

### Step 2

Example 836 (0.14 g, 0.3 mmol) prepared in Step 1 was added CH₂Cl₂ (2 mL) and HCl/Et₂O (2N HCl in Et₂O, 2 mL). Stirred at room temperature for 18 h and then concentrated in vacuo to provide Example 837 as an HCl salt.

### Preparation of 2-formyl-5-cyanopyridine

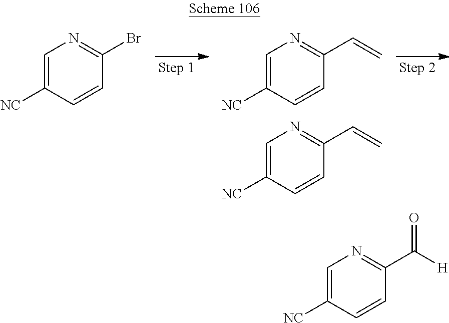

### Step 1

Nitrogen was bubbled through a solution of 2-bromo-5-cyanopyridine (6.0 g, 33 mmol) in MeOH (25 mL) for 5 minutes. Potassium vinyltrifluoroborate (5.3 g, 39 mmol) and TEA (4.5 mL, 33 mmol) were added followed by Pd(dppf)₂Cl.CH₂Cl₂ (1.1 g, 0.04 mmol). Warmed the reaction to 80° C. in a sealed tube and stirred for 8 h. Cooled to room temperature and concentrated in vacuo. Added water and EtOAc and filtered through a bed of Celite. Washed the filtrate with water and brine. Dried the organic layer (MgSO₄), filtered, and concentrated in vacuo. Purified the residue by silica gel chromatography (0-20% EtOAc/Hex over 30 minutes) to proved the olefin (4.1 g, 31.5 mmol).

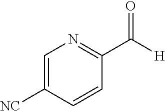

### Step 2

A DCM solution (160 mL) of containing the aldehyde from step 1 (4.1 g, 31 mmol) was cooled to −78° C. Ozone was bubbled through the reaction mixture until the solution turned light blue (−30 minutes). The reaction was then purged with oxygen and then dimethylsulfide (7 mL, 95 mmol) was added. The reaction was stirred for 18 h after taking the cold bath away. Washed the mixture with water and brine. Dried (MgSO₄) the organic layer, filtered, and concentrated in vacuo. The residue was purified by silica gel chromatography (0-20% EtOAc/Hex) to provide the aldehyde (1.3 g, 9.7 mmol).

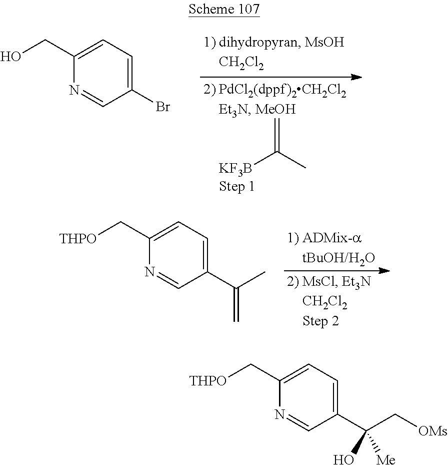

### Step 1:

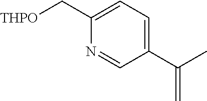

To a solution of 5-bromopyridin-2-yl methanol (Supplier: Biofine International: Vancouver, Canada) (5.27 g, 28.0 mmol) in CH₂Cl₂ was added methanesulfonic acid (2.82 g, 29.4 mmol) and dihydropyran (4.00 g, 47.6 mmol). The resultant solution was stirred at RT overnight. The solution was then washed with NaHCO₃ (aq.), dried over Na₂SO₄, filtered and concentrated. The crude product was purified via flash chromatography (SiO₂: gradient elution, 100:0 to 70:30 hexanes:EtOAc) to afford the ether (6.90 g) as a light yellow oil.

To a solution of the tetrahydropyranyl ether (10.4 g, 38.2 mmol) in MeOH (50 mL) in a pressure tube was added potassium trifluoro(prop-1-en-2-yl)borate (J. Am. Chem. Soc 2003, 125, 11148-11149) (8.5 g, 57 mmol). The resultant slurry was degassed by bubbling N₂ through the solvent for 10 min. To this slurry was added PdCl₂(dppf)₂.CH₂Cl₂ (1.3 g, 1.6 mmol) and Et₃N (3.87 g, 38.2 mmol). The pressure tube was sealed and the mixture was heated to 100° C. with stirring for 16 h. The mixture was then cooled to RT, transferred to a round bottom flask and concentrated. The residue was partitioned between water and CH₂Cl₂. The aqueous layer was extracted with CH₂Cl₂ (3×). The combined organic layers were dried over Na₂SO₄, filtered and concentrated. The crude product was purified via flash chromatography (SiO₂: gradient elution, 100:0 to 65:35 hexanes:EtOAc) to afford the styrene (5.0 g).

### Step 2:

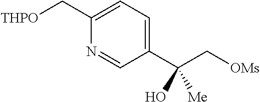

To a biphasic mixture of the styrene from step 1 (2.8 g, 12 mmol) in 1:1 tert-butanol/water (50 mL) was added AD mix α (Aldrich) (17 g) and methane sulfonamide (1.1 g, 12 mmol). The mixture was stirred vigorously at RT for 72 h. At that time, Na₂SO₃ (9.0 g, 72 mmol) was added and the resultant mixture was stirred at RT for 1 h. The mixture was then diluted with 2-propanol and stirred for an additional 1 h. The mixture was filtered through filter paper to remove the solids. The organic layer was then separated, dried over Na₂SO₄, filtered and concentrated. The residue was dissolved in CH₂Cl₂(ca 10 mL). To this solution was added Et₃N (1.8 g, 18 mmol) followed by methanesulfonyl chloride (1.5 g, 12 mmol). The resultant solution was stirred at RT for 48 h. The solution was then diluted with CH₂Cl₂, washed with water, dried over Na₂SO₄, filtered and concentrated. The crude product was purified via flash chromatography (SiO₂: gradient elution, 100:0 to 0:100 hexanes:EtOAc) to afford the mesylate (1.5 g, 36% for 2 steps).

The following halides were converted to mesylates using a similar method to that described in Scheme 107.

TABLE XXVII

| | Halides | |
|---|---|---|
| Entry | (Supplier) | Mesylate |
| 1 | 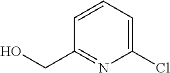 | 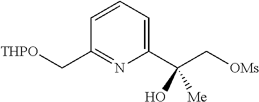 |
| 2 | 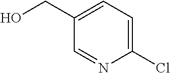 | 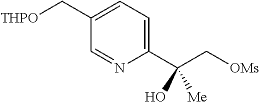 |
| 3 | 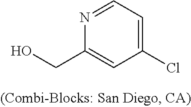 | 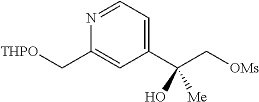 |
| 4 | 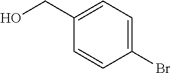 | 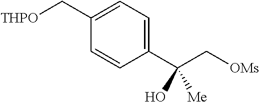 |

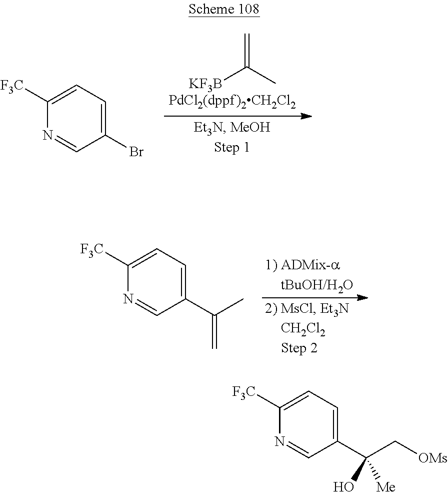

### Step 1:

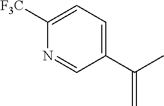

To a solution of 5-bromo-2-trifluoromethylpyridine (4.0 g, 18 mmol) in MeOH (10 mL) in a pressure tube was added potassium trifluoro(prop-1-en-2-yl)borate (3.1 g, 21 mmol). The resultant slurry was degassed by bubbling N₂ through the solvent for 10 min. At that time, PdCl₂(dppf)₂.CH₂Cl₂ (0.58 g, 0.71 mmol) and Et₃N (1.8 g, 18 mmol) were added, the pressure tube was sealed and the mixture was heated to 100° C. with stirring for 3 h. The mixture was then cooled to RT, transferred to a round bottom flask and concentrated in vacuo. The crude residue was partitioned between water and CH₂Cl₂. The aqueous layer was then extracted with CH₂Cl₂ (3×). The combined organic layers were dried over Na₂SO₄, filtered and concentrated. The crude product was purified via flash chromatography (SiO₂: gradient elution, 100:0 to 85:15 hexanes:EtOAc) to afford the styrene (2.5 g, 75%).

### Step 2:

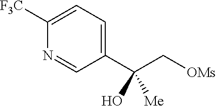 [SMILES: C[C@@](O)(COS(C)(=O)=O)c1ccc(C(F)(F)F)nc1]

To a biphasic mixture of the styrene from Step 1 (2.5 g, 14 mmol) in 1:1 tert-butanol/water (50 mL) was added AD mix α (Aldrich) (19 g) and methane sulfonamide (1.3 g, 14 mmol). The resultant mixture was stirred vigorously at RT for 72 h. After that time, Na₂SO₃ (21 g, 165 mmol) was added and the mixture was stirred at RT for 1 h. The mixture was then diluted with 2-propanol and stirred for 1 h, at which time, the solids were removed via filtration. The organic layer was separated, dried over Na₂SO₄, filtered and concentrated. The residue was redissolved in CH₂Cl₂ (ca 10 mL). To this solution was added Et₃N (1.65 g, 16.3 mmol) followed by methanesulfonyl chloride (1.7 g, 15 mmol). The solution was stirred at RT for 3 h. At that time, the solution was concentrated and the crude product was purified via flash chromatography (SiO₂: gradient elution, 100:0 to 45:55 hexanes:EtOAc) to afford the mesylate (3.5 g, 87% for 2 steps).

The following bromides were converted to mesylates using a similar method to that described in Scheme 108.

TABLE XXVIII

| Entry | halide | Mesylate |
|---|---|---|
| 1 | 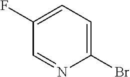 | 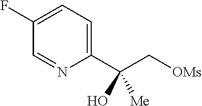 [SMILES: C[C@@](O)(COS(C)(=O)=O)c1ccc(F)cn1] |
| 2 | 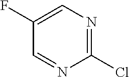 | 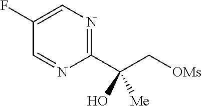 [SMILES: C[C@@](O)(COS(C)(=O)=O)c1ncc(F)cn1] |
| 3 | 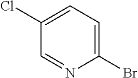 | 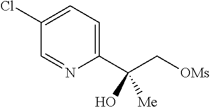 [SMILES: C[C@@](O)(COS(C)(=O)=O)c1ccc(Cl)cn1] |
| 4 | 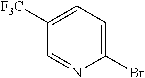 [SMILES: FC(F)(F)c1ccc(Br)nc1] | 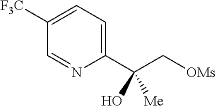 [SMILES: C[C@@](O)(COS(C)(=O)=O)c1ccc(C(F)(F)F)cn1] |
| 5 | 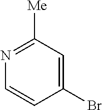 | 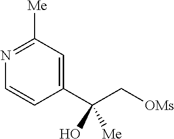 [SMILES: Cc1cc([C@](C)(O)COS(C)(=O)=O)ccn1] |
| 6 | 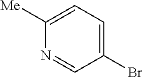 | 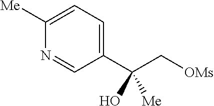 [SMILES: Cc1ccc([C@](C)(O)COS(C)(=O)=O)cn1] |
| 7 | 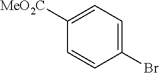 [SMILES: COC(=O)c1ccc(Br)cc1] | 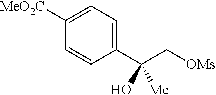 [SMILES: COC(=O)c1ccc([C@](C)(O)COS(C)(=O)=O)cc1] |
| 8 | 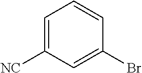 | 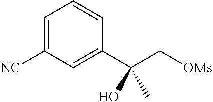 [SMILES: C[C@@](O)(COS(C)(=O)=O)c1cccc(C#N)c1] |
| 9 | 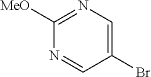 | 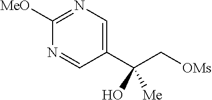 [SMILES: COc1ncc([C@](C)(O)COS(C)(=O)=O)cn1] |
| 10 | 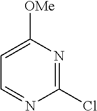 | 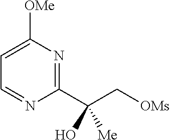 [SMILES: COc1ccnc([C@](C)(O)COS(C)(=O)=O)n1] |
| 11 | 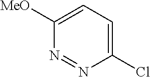 | 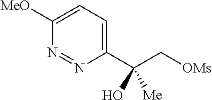 [SMILES: COc1ccc([C@](C)(O)COS(C)(=O)=O)nn1] |
| 12 | 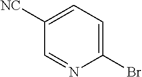 | 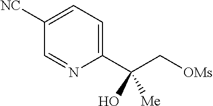 [SMILES: C[C@@](O)(COS(C)(=O)=O)c1ccc(C#N)cn1] |
| 13 | 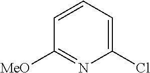 | 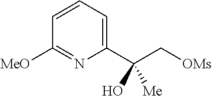 [SMILES: COc1cccc([C@](C)(O)COS(C)(=O)=O)n1] |
| 14 | 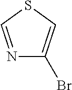 | 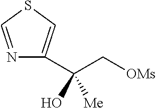 [SMILES: C[C@@](O)(COS(C)(=O)=O)c1cscn1] |
| 15** | 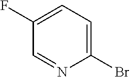 | 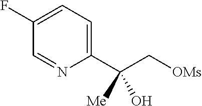 [SMILES: C[C@](O)(COS(C)(=O)=O)c1ccc(F)cn1] |

**Entry 15 was prepared using a method similar to that described in Scheme 108,
except AD mix β was used in step 2 instead of AD mix α.

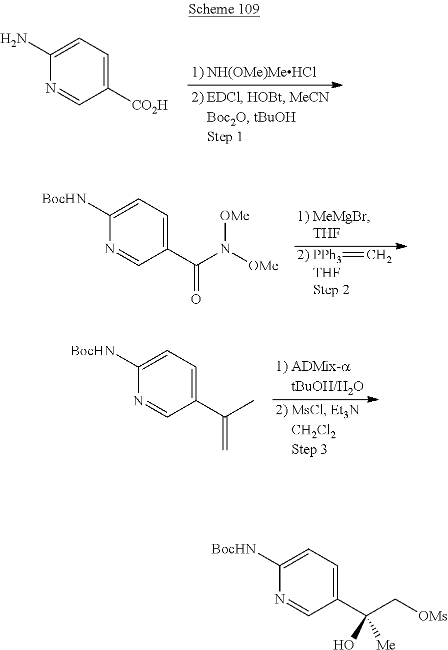

### Step 1:

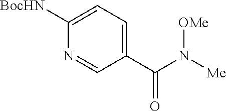 [SMILES: CON(C)C(=O)c1ccc(NC(=O)OC(C)(C)C)nc1]

To a slurry of 6-aminonicotinic acid (12.5 g, 90.5 mmol) in MeCN (150 mL) was added N,O-dimethylhydroxylamine hydrochloride (10.6 g, 109 mmol), HOBt (14.7 g, 109 mmol), EDCI (20.8 g, 109 mmol) and diisopropylethylamine (35.0 g, 272 mmol). The resultant mixture was stirred at RT overnight. Once the reaction was complete, the mixture was concentrated in vacuo. The residue was partitioned between 1 M NaOH (aq.) and EtOAc and the aqueous layer was extracted with EtOAc (3×). The combined organic layers were washed with brine, dried over Na₂SO₄, filtered and concentrated to afford the amide (6.7 g) as a white solid. The product was slurried in tert-butanol (100 mL) and di-tert-butyldicarbonate (8.88 g, 40.7 mmol) was added. The resultant mixture was stirred at RT overnight. Additional di-tert-butyldicarbonate (1.5 g, 6.9 mmol) was added and the mixture was stirred at RT for an additional 48 h. The reaction mixture was then concentrated to afford the amide (9.6 g, 38% yield for 2 steps) as a tan solid that was used without further purification.

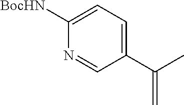 [SMILES: C=C(C)c1ccc(NC(=O)OC(C)(C)C)nc1]

### Step 2:

To a solution of the amide from step 1 (9.6 g, 34 mmol) in THF (200 mL) at 0° C. was added a solution of MeMgBr (3 M in hexanes, 28.4 mL, 85 mmol). The solution was stirred at 0° C. for 1 h. At that time, 1 M HCl (aq.) was slowly added and the biphasic mixture was extracted with EtOAc (3×). The combined organic layers were washed sequentially with NaHCO₃ (aq.) and brine, dried over Na₂SO₄, filtered and concentrated to afford the ketone (8.0 g) as a tan solid.

To a slurry of methyltriphenylphosphonium bromide (24 g, 68 mmol) in THF (150 mL) was added dropwise a solution of n-BuLi (1.6 M in hexanes, 42.3 mL, 68 mmol). The mixture was stirred at RT for 1 h then cooled to 0° C. A solution of the ketone from above (8.0 g, 34 mmol) in THF (150 mL) was added slowly via addition funnel to the mixture. Once the addition was complete, the mixture was warmed to RT and stirred. After 16 h at RT, water was added and the aqueous layer was extracted with EtOAc (3×). The combined organic layers were washed with brine, dried over Na₂SO₄, filtered and concentrated. The crude product was purified via flash chromatography (SiO₂: gradient elution, 100:0 to 60:40 hexanes:EtOAc) to afford the styrene (6.4 g, 80% for 2 steps) as an off white solid.

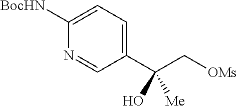 [SMILES: CC(C)(C)OC(=O)Nc1ccc([C@](C)(O)COS(C)(=O)=O)cn1]

### Step 3:

The mesylate was prepared using a similar procedure to that described in Scheme 108 step 2 except the styrene from Step 2 of this example was used.

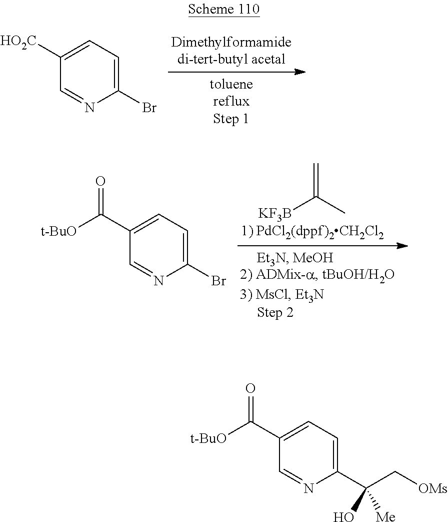

### Step 1:

To a solution of the 6-bromonicotinic acid (2.5 g, 12.4 mmol) in toluene (25 mL) was added dimethylformamide di-tert-butylacetyl (5.0 g, 24.8 mmol). The solution was then heated to reflux overnight. Additional dimethylformamide di-tert-butylacetal (10.0 g, 59.6 mmol) was added in two portions over 24 h with continued stirring at reflux. The solution was stirred at reflux for a total of 72 h then cooled to RT. To the solution was added sat. NaHCO₃ (aq.) and the aqueous layer was extracted with EtOAc (3×). The combined organic layers were washed with brine, dried over Na₂SO₄, filtered and concentrated. The crude product was purified via flash chromatography (SiO₂: gradient elution, 100:0 to 92:8 hexanes:EtOAc) to afford the tert-butyl ester (1.68 g, 52%).

### Step 2:

The mesylate was prepared using a similar method to that described in Scheme 108 step 2 except the bromide from step 1 of this scheme was used.

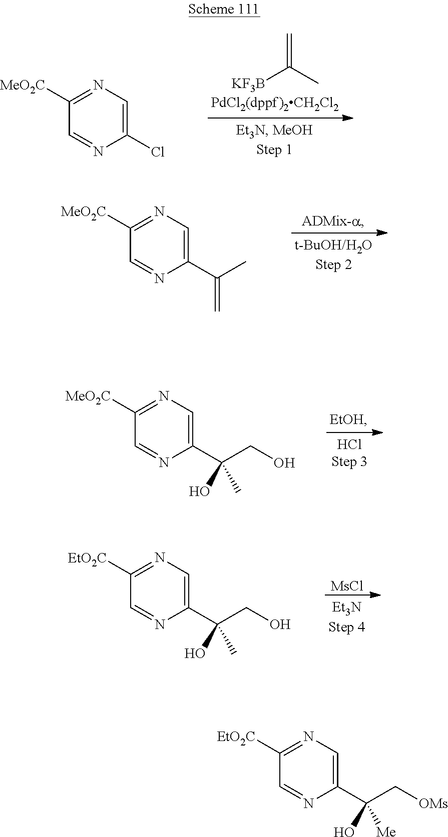

### Step 1:

The styrene was prepared from methyl 5-chloropyrazine-2-carboxylate (Lonza Inc, Allendale, N.J.) using a procedure similar to that described in Scheme 108 Step 1.

### Step 2:

The diol carboxylic acid was prepared using a procedure similar to that described in Scheme 108 Step 2 except the diol from step 1 of this scheme was used.

### Step 3:

To a solution of the carboxylic acid from step 2 (ca 6.5 mmol) in anhydrous EtOH (30 mL) was added a solution of hydrogen chloride (4 M in dioxane, 5 mL). The solution was heated to reflux with stirring for 4 h. After that time, the solution was allowed to cool to RT. The solution was then concentrated in vacuo and used without further purification.

### Step 4:

To a solution was the diol from Step 3 (ca 6.5 mmol) in CH₂Cl₂ (20 mL) was added Et₃N (1.4 g, 14.3 mmol) followed by methane sulfonylchloride (825 mg, 7.2 mmol). The solution was stirred at RT. After the reaction was complete, the solution was diluted with CH₂Cl₂ and washed with NaHCO₃ (aq.). The organic layer was dried over Na₂SO₄, filtered and concentrated. The crude product was purified via flash chromatography (SiO₂: gradient elution, 100:0 to 50:50 hexanes:EtOAc) to afford the mesylate (1.45 g, 81%) as a clear oil.

### Preparation of Olefin

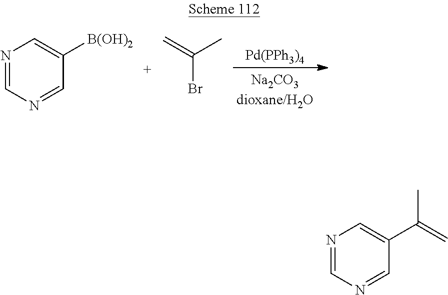

The boronic acid (3.1 g, 25 mmol), 2-bromopropene (3.3 ml), Pd(PPh₃)₄ (720 mg), and Na₂CO₃ (4 g) were taken up in dioxane/H₂O (4/1, 40 ml), and the resulting solution was heated in a sealed tube overnight at 85° C. The solution was partitioned between EtOAc and water. The aqueous layer was extracted with EtOAc. The combined organic layers were washed with brine and dried (MgSO₄). Filtration and concentration gave a brown oil. The residue was purified via gradient flash chromatography (0-30% EtOAc in hexanes, SiO₂) which provided 1.1 g (37%) of the olefin as a yellow oil.

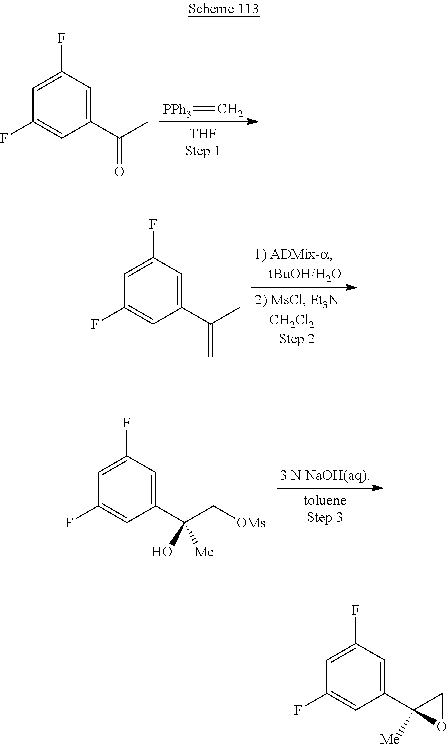

### Step 1:

To a slurry of methyltriphenylphosphonium bromide (21.6 g, 57.6 mmol) in THF (100 mL) at 0° C. was added dropwise a solution of n-BuLi (1.6 M in hexanes, 36.0 mL, 57.6 mmol). The mixture was stirred at 0° C. for 30 min. After that time, a solution of 1-(3,5-difluorophenyl)ethanone (6.00 g, 38.4 mmol) in THF (100 mL) was added dropwise via addition funnel. Once the addition was complete, the mixture was warmed to RT and stirred overnight. Water was then added and the aqueous layer was extracted with EtOAc (3×). The combined organic layers were washed with brine, dried over Na₂SO₄, filtered and concentrated. The crude product was purified via flash chromatography (SiO₂: gradient elution, 100:0 to 88:12 hexanes:EtOAc) to afford the styrene (4.3 g, 72%) as a clear oil.

### Step 2:

The mesylate was prepared using a similar method to that described in Scheme 108 Step 3 except the styrene from Step 1 of this scheme was used.

### Step 3:

To a solution of the mesylate from Step 2 (295 mg, 1.1 mmol) in toluene (12 mL) was added 3 N NaOH (aq.) (6 mL). The mixture was stirred vigorously at RT for 3 h. The aqueous layer was extracted with EtOAc (3×). The combined organic layers were washed with brine, dried over Na₂SO₄, filtered and concentrated.

The following ketones or styrenes were converted to a mesylate or epoxide using a similar method to that described in Scheme 113.

TABLE XXX

| Entry | Ketone/olefin | Epoxide/Mesylate |
|---|---|---|
| 1 | 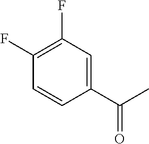 | 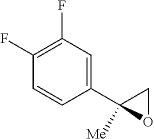 [SMILES: C[C@]1(c2ccc(F)c(F)c2)CO1] |
| 2 | 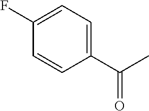 | 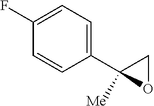 [SMILES: C[C@]1(c2ccc(F)cc2)CO1] |
| 3 | 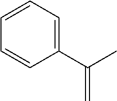 | 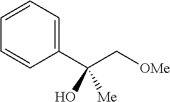 [SMILES: COC[C@@](C)(O)c1ccccc1] |
| 4 | 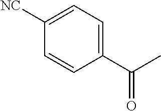 | 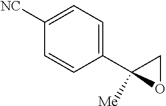 [SMILES: C[C@]1(c2ccc(C#N)cc2)CO1] |
| 5 | 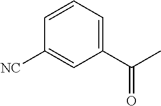 | 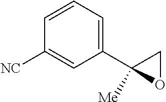 [SMILES: C[C@]1(c2cccc(C#N)c2)CO1] |
| 6 | 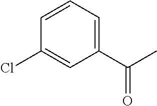 | 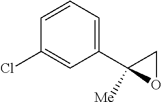 [SMILES: C[C@]1(c2cccc(Cl)c2)CO1] |
| 7 | 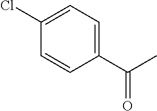 | 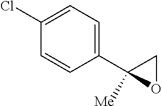 [SMILES: C[C@]1(c2ccc(Cl)cc2)CO1] |
| 8 | 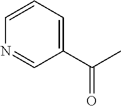 | 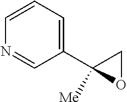 |
| 9 | 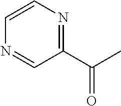 | 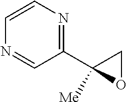 |
| 10 | 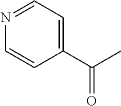 | 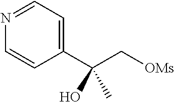 |
| 11 | 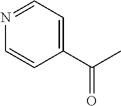 | 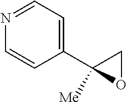 |
| 12 | 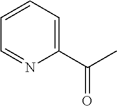 | 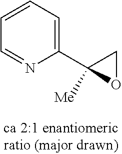 |
| 13 | 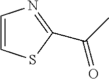 | 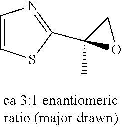 |
| 14 | 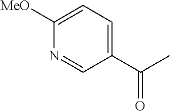 | 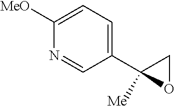 [SMILES: COc1ccc([C@@]2(C)CO2)cn1] |
| 15 | 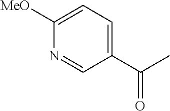 | 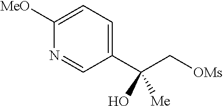 [SMILES: COc1ccc([C@](C)(O)COS(C)(=O)=O)cn1] |
| 16 | 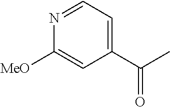 | 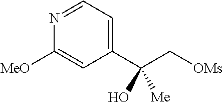 [SMILES: COc1cc([C@](C)(O)COS(C)(=O)=O)ccn1] |
| 17 | 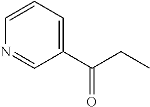 | 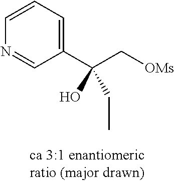 [SMILES: CC[C@@](O)(COS(C)(=O)=O)c1cccnc1] |
| 18 | 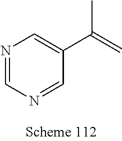 | 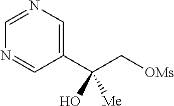 [SMILES: C[C@@](O)(COS(C)(=O)=O)c1cncnc1] |
| 19 | 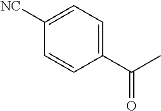 | 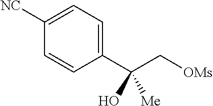 [SMILES: C[C@@](O)(COS(C)(=O)=O)c1ccc(C#N)cc1] |
| 20 | 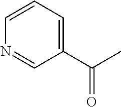 | 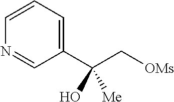 [SMILES: C[C@@](O)(COS(C)(=O)=O)c1cccnc1] |
| 21 | 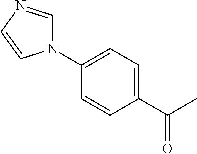 | 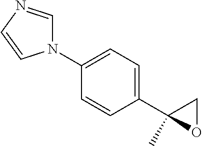 |

The following epoxides or mesylates were prepared using a method similar to that described in Scheme 113 except AD-mix β was used in Step 2 instead of AD-mix α.

TABLE XXXI

| Entry | Ketone | Epoxide |
|---|---|---|
| 1 | 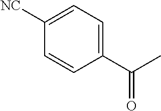 | 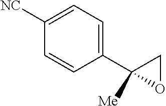 [SMILES: C[C@@]1(c2ccc(C#N)cc2)CO1] |
| 2 | 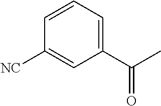 | 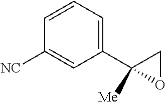 [SMILES: C[C@@]1(c2cccc(C#N)c2)CO1] |
| 3 | 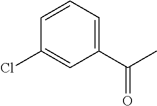 | 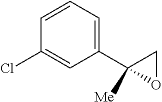 [SMILES: C[C@@]1(c2cccc(Cl)c2)CO1] |
| 4 | 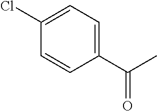 | 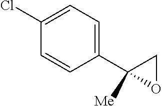 [SMILES: C[C@@]1(c2ccc(Cl)cc2)CO1] |
| 5 | 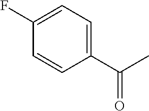 | 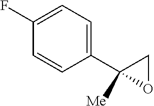 [SMILES: C[C@@]1(c2ccc(F)cc2)CO1] |
| 6 | 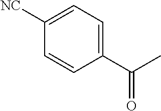 | 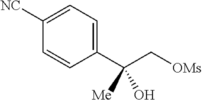 [SMILES: C[C@](O)(COS(C)(=O)=O)c1ccc(C#N)cc1] |
| 7 | 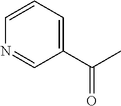 | 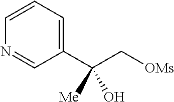 [SMILES: C[C@](O)(COS(C)(=O)=O)c1cccnc1] |
| 8 | 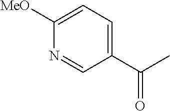 | 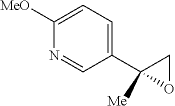 [SMILES: COc1ccc([C@]2(C)CO2)cn1] |

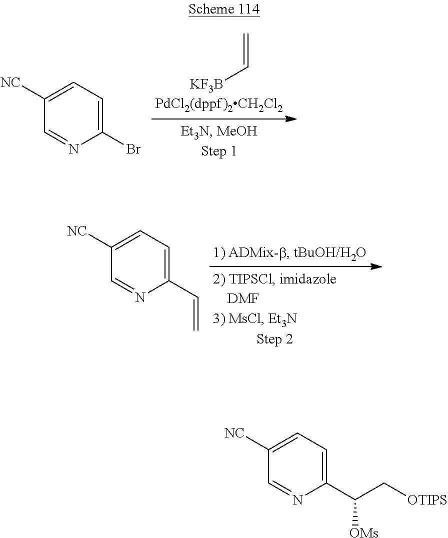

### Step 1:

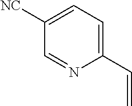

To a solution of the 2-bromo-5-cyanopyridine (6.37 g, 34.8 mmol) in MeOH (25 mL) in a pressure tube was added vinyl trifluoroborane (Aldrich) (5.60 g, 41.8 mmol). The resultant slurry was degassed by bubbling N₂ through the solvent for 10 min. To this slurry was added PdCl₂(dppf)₂.CH₂Cl₂ (1.14 g, 1.40 mmol) and Et₃N (3.51 g, 34.8 mmol). The pressure tube was sealed and the mixture was heated to 80° C. with stirring for 8 h. The mixture was then cooled to RT, transferred to a round bottom flask and concentrated in vacuo. The residue was partitioned between water and EtOAc. The aqueous layer was extracted with EtOAc (3×). The combined organic layers were washed with brine, dried over Na₂SO₄, filtered and concentrated. The crude product was purified via flash chromatography (SiO₂: gradient elution, 100:0 to 80:20 hexanes:EtOAc) to afford the styrene (4.50 g, 99%).

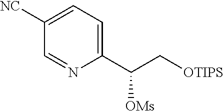

### Step 2:

To a biphasic mixture of the styrene from Step 1 (4.50 g, 34.5 mmol) in 1:1 tert-butanol/water (150 mL) was added AD mix β (Aldrich) (48 g) and methane sulfonamide (3.3 g, 34.5 mmol). The mixture was stirred vigorously at RT for 24 h. After that time, Na₂SO₃ (50 g) was added and the mixture was stirred at RT for 1 h. The mixture was then diluted with 2-propanol and filtered through filter paper. The organic layer was separated, dried over Na₂SO₄, filtered and concentrated. The crude product was purified via flash chromatography (SiO₂: gradient elution, 100:0 to 95:5 CH₂Cl₂: MeOH) to afford the diol (4.40 g, 78%).

To a portion of the diol (1.17 g, 7.10 mmol) in DMF (10 mL) was added triisopropylsilyl chloride (1.37 g, 7.1 mmol) and imidazole (1.21 g, 17.8 mmol). The resultant solution was stirred at RT for 24 h. After that time, the solution was diluted with Et₂O and washed with water (2×). The organic layer was dried over Na₂SO₄, filtered and concentrated. The crude product was purified via flash chromatography (SiO₂: gradient elution, 100:0 to 85:15 hexanes:EtOAc) to afford the silyl ether (1.60 g, 70%) as a white crystalline solid. To a solution of the ether (1.60 g) in CH₂Cl₂ (25 mL) was added Et₃N (0.758 g, 7.50 mmol) followed by methanesulfonyl chloride (0.600 g, 5.20 mmol). After stirring at RT for 3 h, the solution was diluted with CH₂Cl₂ and washed with NaHCO₃ (aq.). The aqueous layer was back extracted with CH₂Cl₂ (2×). The combined organic layers were dried over Na₂SO₄, filtered and concentrated. The crude product was purified via flash chromatography (SiO₂: gradient elution, 100:0 to 75:25 hexanes:EtOAc) to afford the mesylate (1.34 g, 67%) as a mixture of enantiomers (ca 6:1 with enantiomer pictured above the major).

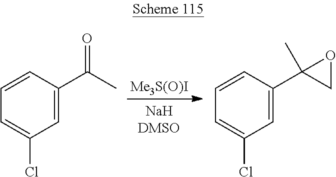

To a slurry of trimethyl sulfoxonium iodide (3.41 g, 15.5 mmol) in anhydrous DMSO (30 mL) was added NaH (60% in oil; 620 mg, 15.5 mmol). The mixture was stirred at RT for 30 min. To the mixture was added a solution of 3-chloroacetophenone (2.0 g, 12.9 mmol) in anhydrous DMSO (20 mL). The resultant mixture was stirred at RT overnight. Water was added to the mixture. The mixture was then extracted with Et₂O (3×). The combined organic layers were washed with brine, dried over Na₂SO₄, filtered and concentrated. The crude product was purified via flash chromatography (SiO₂: gradient elution, 100:0 to 85:15 hexanes:EtOAc) to afford the epoxide (1.80 g) as a clear oil.

The following acetophenones were prepared using a similar procedure to that described in Scheme 115.

TABLE XXXII

| Entry | Ketone | Epoxide |
|---|---|---|
| 1 | 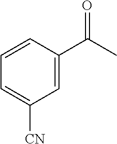 | 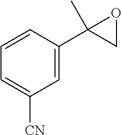 |
| 2 | 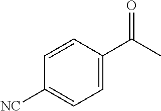 | 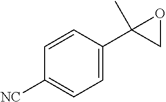 |
| 3 | 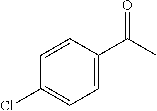 | 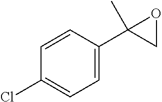 |
| 4 | 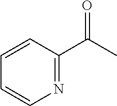 | 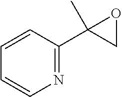 |
| 5 | 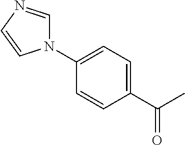 | 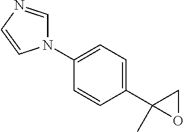 |
| 6 | 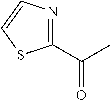 | 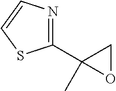 |

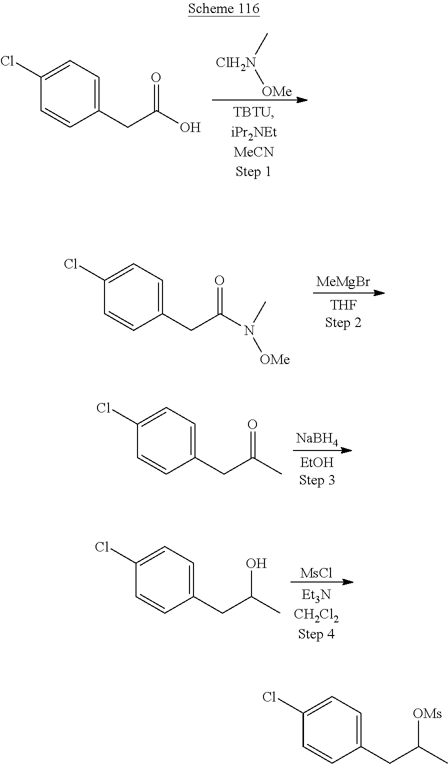

### Step 1:

To a solution of 2-(4-chlorophenyl)acetic acid (1.0 g, 5.9 mmol) in MeCN (15 mL) was added TBTU (2.25 g, 7.0 mmol), N,O-dimethylhydroxylamine hydrochloride (0.69 g, 7.0 mmol) and diisopropylethylamine (3.03 g, 23.4 mmol). The resultant solution was stirred at RT overnight. The solvent was removed in vacuo. The residue was partitioned between EtOAc and 1 M HCl (aq.) and the layers were separated. The organic layer was washed with sat NaHCO₃ (aq.) and brine. The organic layer was then dried over Na₂SO₄, filtered and concentrated to afford the amide (ca 1.0 g).

### Step 2:

To a solution of the amide from step 1 (0.93 g, 5.9 mmol) in THF at 0° C. was added a solution of methyl magnesium bromide (3 M in hexanes, 7.8 mmol). The resultant mixture was stirred at RT for 1 hours. To this mixture was added 1 M HCl (aq.). The aqueous layer was extracted with EtOAc (3×). The combined organic layers were dried over Na₂SO₄, filtered and concentrated. The crude product was purified via flash chromatography (SiO₂: gradient elution, 100:0 to 85:15 hexanes:EtOAc) to afford the ketone (0.93 g).

### Step 3:

To a solution of the ketone from step 2 (0.5 g, 3.0 mmol) in anhydrous EtOH (5 mL) was added sodium borohydride (150 mg, 3.9 mmol). The resultant mixture was stirred at RT for 1 hour. Water was added and the mixture was extracted with EtOAc (3×). The combined organic layers were dried over Na₂SO₄, filtered and concentrated to afford the alcohol (0.3 g) that was used without further purification.

### Step 4:

To a solution of the alcohol from step 3 (0.3 g, 1.8 mmol) in CH₂Cl₂ (30 mL) was added triethylamine (0.21 g, 2.1 mmol) and methanesulfonyl chloride (0.24 g, 2.1 mmol). The resultant mixture was stirred at RT for 2 hours. The water was added to the mixture and the layers were separated. The organic layer was dried over Na₂SO₄, filtered and concentrated. The crude product was purified via flash chromatography (SiO₂: gradient elution, 100:0 to 75:25 hexanes:EtOAc) to afford the mesylate (0.38 g).

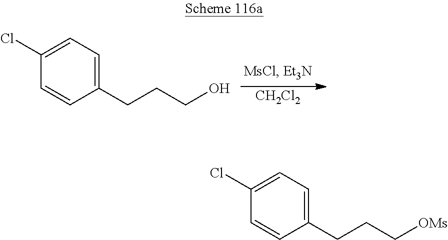

To a solution of 3-(4-chlorophenyl)propan-1-ol (215 mg, 1.26 mmol) in CH₂Cl₂ (3 mL) was added triethylamine (153 mg, 1.51 mmol) and methanesulfonyl chloride (173 mg, 1.51 mmol). The resultant solution was stirred at RT for 16 hours. The mixture was diluted with CH₂Cl₂ and the mixture was washed with water. The organic layer was dried over Na₂SO₄, filtered and concentrated. The mesylate was used as is without further purification.

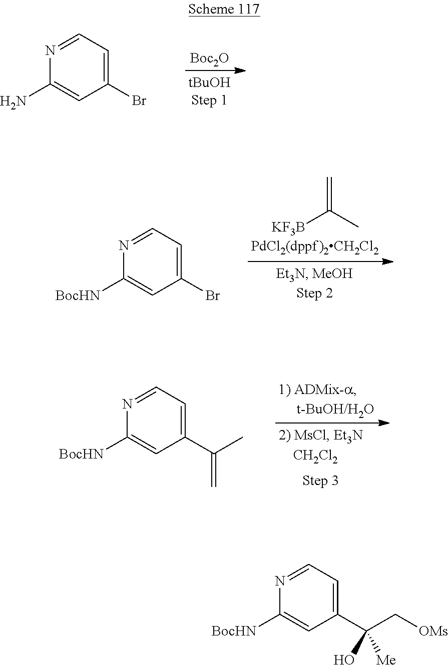

### Step 1:

To a solution of 4-bromopyridin-2-amine (1.0 g, 5.8 mmol) in t-BuOH was added di-tert-butyldicarbonate. The resultant solution was stirred at RT for 48 hours. The solvent was removed in vacuo to afford the carbamate (1.6 g) as an off white solid.

### Step 2:

The styrene was prepared using a method similar to that described in Scheme 108 step 1.

### Step 3:

The mesylate was prepared using a method similar to that described in Scheme 108 step 2.

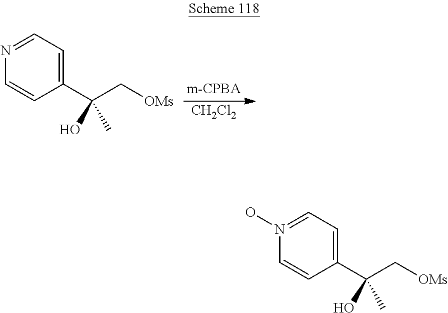

To a solution of the mesylate from Table XXX entry 10 (150 mg, 0.65 mmol) in CH₂Cl₂ at 0° C. was added m-CPBA (120 mg, 0.71 mmol). The resulting solution was stirred at 0° C. for 30 min. The solution was then heated to reflux overnight. The mixture was then concentrated and used without further purification.

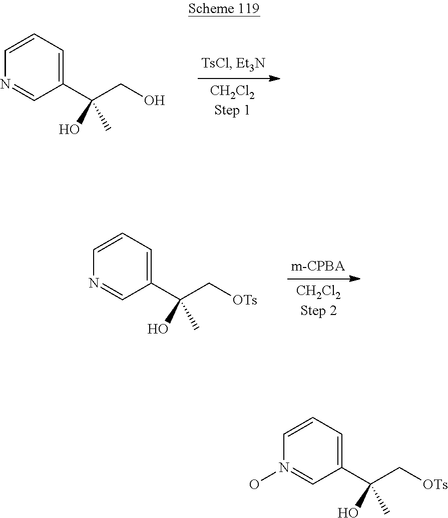

### Step 1:

To a solution of the diol (135 mg, 0.88 mmol) in CH₂Cl₂ (4 mL) was added Et₃N (107 mg, 1.06 mmol) and p-toluenesulfonyl chloride (201 mg, 1.06 mmol). The resultant mixture was stirred at RT overnight. After that time, additional Et₃N (53 mg, 0.55 mmol) and p-toluenesulfonyl chloride (100 mg, 0.55 mmol) were added and the mixture was stirred at RT for 2.5 days. The mixture was then diluted with CH₂Cl₂ and washed with water. The aqueous layer was extracted with CH₂Cl₂ (3×). The combined organic layers were dried over Na₂SO₄, filtered and concentrated. The crude product was purified via preparative TLC (SiO₂: 95:5:1 CH₂Cl₂:MeOH: 7 N NH₃ (in MeOH) to afford the tosylate (90 mg).

### Step 2:

To a solution of the tosylate from step 1 (90 mg, 0.29 mmol) in CH₂Cl₂ (5 mL) at 0° C. was added m-CPBA (80 mg, 0.32 mmol). The resulting solution was stirred at 0° C. for 30 min. The solution was then heated to reflux overnight. The solution was concentrated to approximately ⅓ of the initial volume. A white precipitate formed. Methanol (2 drops) was added to the mixture to dissolve the solid. The crude material was then purified via flash chromatography [SiO₂: 95:5:1 CH₂Cl₂:MeOH: 7 N NH₃ (in MeOH)] to afford the N-oxide (73 mg).

### Preparation of Example 839 and Example 840

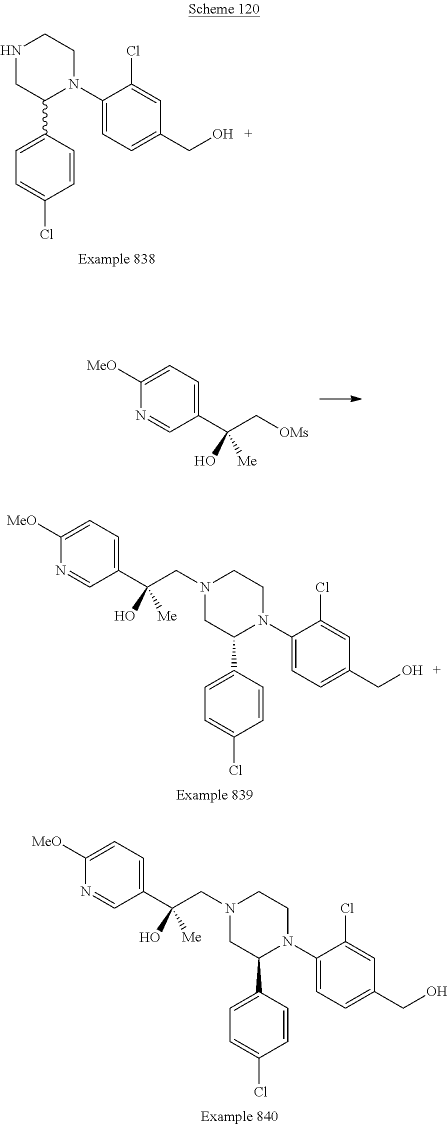

Example 838 was prepared in the same manner as Example 810 in scheme 93 except that in step 1, Example 305 was treated with concentrated hydrochloric acid at 100° C. for 18 h and then concentrated in vacuo instead of 3N NaOH at 90° C. for 18 h.

To Example 838 (0.08 g, 0.24 mmol) in ethanol (1 mL) was added the mesylate from table XXX entry 15 (0.068 g, 0.26 mmol) and sodium carbonate (0.027 g, 0.26 mmol). The reaction mixture was warmed to 80° C. and stirred for 19 h. The reaction was cooled to room temperature and then concentrated in vacuo. Water was added and the mixture was extracted with EtOAc. The organic layer was washed with water and brine, dried (MgSO₄), filtered, and concentrated in vacuo. The residue was passed through a silica gel column (0-65% EtOAc/hex over 30 minutes) to provide a mixture of diastereomers. The diastereomers were separated by preparative chiral HPLC [Chiralcel OD, 85% hexane/IPA, 254 nm, 1 mL/min.] to provide Example 839 (0.056 g). The eluent was then changed to 75% hex/IPA to obtain Example 840 (0.04 g).

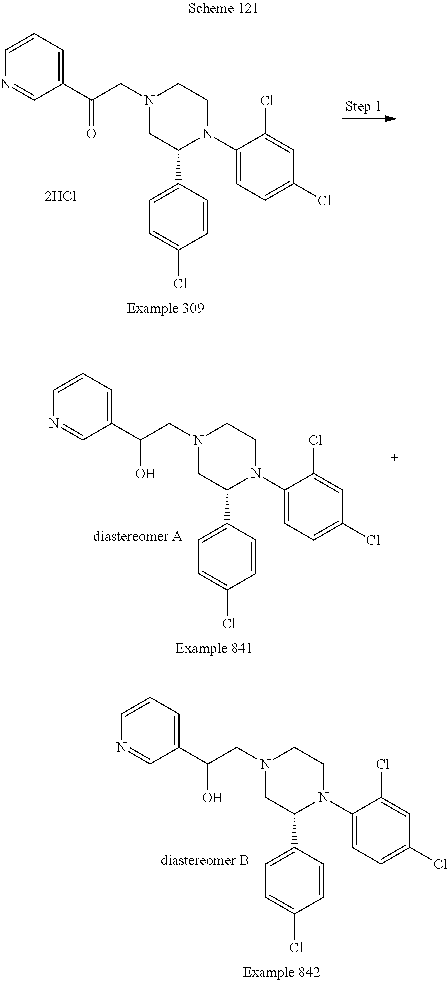 [SMILES: O=C(CN1CCN(c2ccc(Cl)cc2Cl)[C@H](c2ccc(Cl)cc2)C1)c1cccnc1 ; OC(CN1CCN(c2ccc(Cl)cc2Cl)[C@H](c2ccc(Cl)cc2)C1)c1cccnc1 ; "+" ; OC(CN1CCN(c2ccc(Cl)cc2Cl)[C@H](c2ccc(Cl)cc2)C1)c1cccnc1]

To Example 309 (0.26 g, 0.49 mmol) in MeOH (2.5 mL) at 0 C was added NaBH₄ (0.03 g, 0.86 mmol) [Gas evolution]. Took the cold bath away and stirred for 18 h. Concentrated the reaction in vacuo. Added water and extracted with ethyl acetate. Combined the organics and washed with water and brine. Dried (MgSO₄), filtered, and concentrated in vacuo. Purified by silica gel chromatography (20-90% EtOAc/hex over 15 minutes to provide a mixture of Example 841 and Example 842 (0.10 g). Example 841 and Example 842 were separated by HPLC [Chiralcel OD, 80% hex/IPA, 50 ml/min., 254 nm].

### Preparation of Examples 842b and 842c

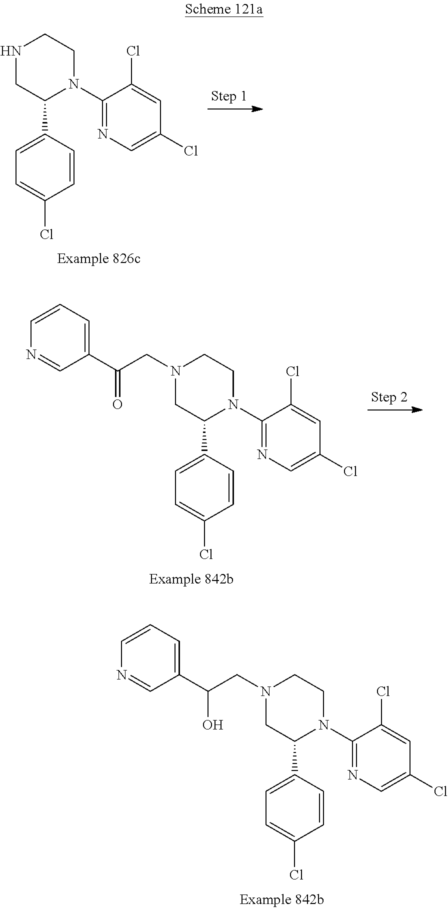 [SMILES: Clc1ccc([C@@H]2CNCCN2c2ncc(Cl)cc2Cl)cc1 ; O=C(CN1CCN(c2ncc(Cl)cc2Cl)[C@H](c2ccc(Cl)cc2)C1)c1cccnc1 ; OC(CN1CCN(c2ncc(Cl)cc2Cl)[C@H](c2ccc(Cl)cc2)C1)c1cccnc1]

Step 1 Example 842b was prepared in the same manner as Example 309 except that the piperazine Example 826c was used instead of Example 304.

Step 2 Example 842b was converted to Example 842c as a mixture of diastereomers using the conditions described in Scheme 121.

### Preparation of Examples 843-845

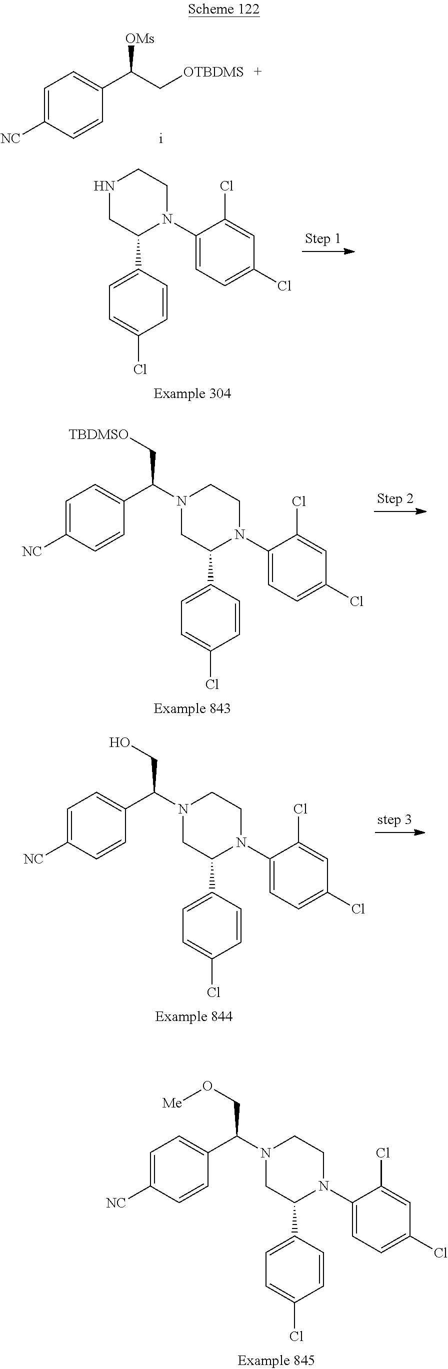 [SMILES: CC(C)(C)[Si](C)(C)OC[C@H](OS(C)(=O)=O)c1ccc(C#N)cc1 ; "+" ; Clc1ccc([C@@H]2CNCCN2c2ccc(Cl)cc2Cl)cc1 ; CC(C)(C)[Si](C)(C)OC[C@H](c1ccc(C#N)cc1)N1CCN(c2ccc(Cl)cc2Cl)[C@H](c2ccc(Cl)cc2)C1 ; N#Cc1ccc([C@@H](CO)N2CCN(c3ccc(Cl)cc3Cl)[C@H](c3ccc(Cl)cc3)C2)cc1 ; COC[C@H](c1ccc(C#N)cc1)N1CCN(c2ccc(Cl)cc2Cl)[C@H](c2ccc(Cl)cc2)C1]

Example 843, Example 844, and Example 845 were prepared in a similar fashion as Examples 771, Example 772, and Example 773 in Scheme 77 except that AD mix β was used instead of AD mix α to prepare the mesylate i.

Examples 846-857 in table XXXIII were prepared by condensing the requisite mesylate (whose synthesis is described in either Scheme 77 Steps 1-3 or Scheme 114) and the requisite core piperazine, followed by cleavage of the silyl protecting group. This two step protocol was executed in a manner similar to that described in Steps 4 and 5 of Scheme 77.

TABLE XXXIII

| Exam- | | | |
|---|---|---|---|
| ple | | | |
| # | Mesylate | Piperazine | Example Structure |
| 846 | 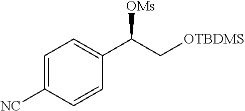 [SMILES: CC(C)(C)[Si](C)(C)OC[C@H](OS(C)(=O)=O)c1ccc(C#N)cc1] | 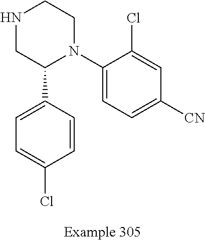 [SMILES: N#Cc1ccc(N2CCNC[C@H]2c2ccc(Cl)cc2)c(Cl)c1] | 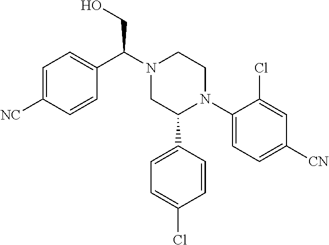 [SMILES: N#Cc1ccc([C@@H](CO)N2CCN(c3ccc(C#N)cc3Cl)[C@H](c3ccc(Cl)cc3)C2)cc1] |
| 847 | 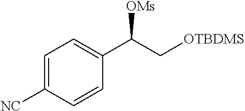 [SMILES: CC(C)(C)[Si](C)(C)OC[C@H](OS(C)(=O)=O)c1ccc(C#N)cc1] | 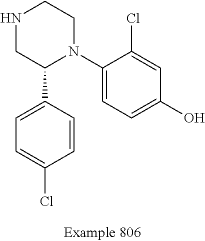 [SMILES: Oc1ccc(N2CCNC[C@H]2c2ccc(Cl)cc2)c(Cl)c1] | 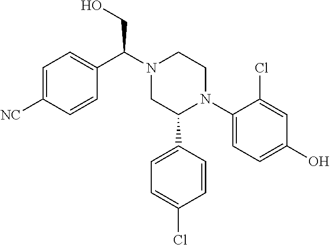 [SMILES: N#Cc1ccc([C@@H](CO)N2CCN(c3ccc(O)cc3Cl)[C@H](c3ccc(Cl)cc3)C2)cc1] |
| 847a | 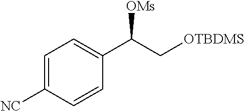 [SMILES: CC(C)(C)[Si](C)(C)OC[C@H](OS(C)(=O)=O)c1ccc(C#N)cc1] | 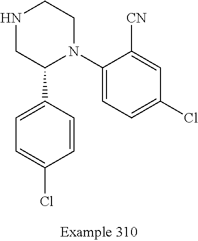 [SMILES: N#Cc1cc(Cl)ccc1N1CCNC[C@H]1c1ccc(Cl)cc1] | 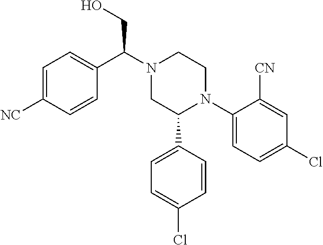 [SMILES: N#Cc1ccc([C@@H](CO)N2CCN(c3ccc(Cl)cc3C#N)[C@H](c3ccc(Cl)cc3)C2)cc1] |
| 848 | 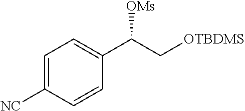 [SMILES: CC(C)(C)[Si](C)(C)OC[C@@H](OS(C)(=O)=O)c1ccc(C#N)cc1] | 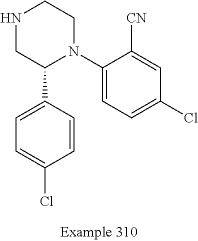 [SMILES: N#Cc1cc(Cl)ccc1N1CCNC[C@H]1c1ccc(Cl)cc1] | 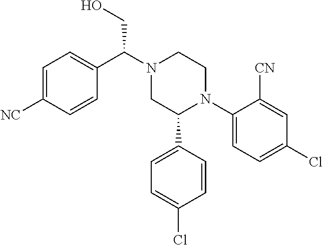 [SMILES: N#Cc1ccc([C@H](CO)N2CCN(c3ccc(Cl)cc3C#N)[C@H](c3ccc(Cl)cc3)C2)cc1] |
| 849 | 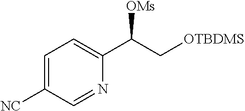 [SMILES: CC(C)(C)[Si](C)(C)OC[C@H](OS(C)(=O)=O)c1ccc(C#N)cn1] | 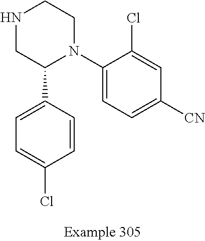 [SMILES: N#Cc1ccc(N2CCNC[C@H]2c2ccc(Cl)cc2)c(Cl)c1] | 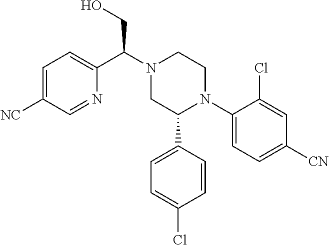 [SMILES: N#Cc1ccc([C@@H](CO)N2CCN(c3ccc(C#N)cc3Cl)[C@H](c3ccc(Cl)cc3)C2)nc1] |
| 850 | 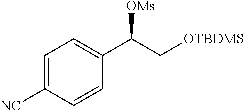 [SMILES: CC(C)(C)[Si](C)(C)OC[C@H](OS(C)(=O)=O)c1ccc(C#N)cc1] | 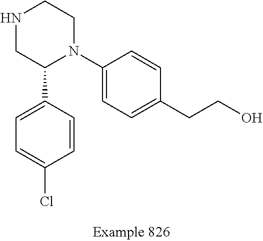 [SMILES: OCCc1ccc(N2CCNC[C@H]2c2ccc(Cl)cc2)cc1] | 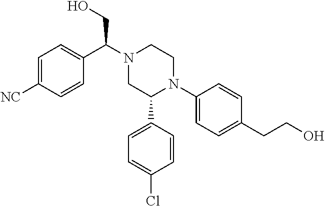 [SMILES: N#Cc1ccc([C@@H](CO)N2CCN(c3ccc(CCO)cc3)[C@H](c3ccc(Cl)cc3)C2)cc1] |
| 851 | 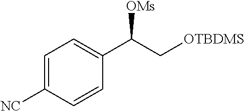 [SMILES: CC(C)(C)[Si](C)(C)OC[C@H](OS(C)(=O)=O)c1ccc(C#N)cc1] | 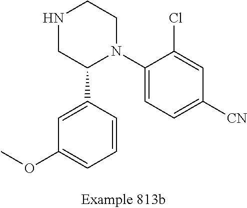 [SMILES: COc1cccc([C@@H]2CNCCN2c2ccc(C#N)cc2Cl)c1] | 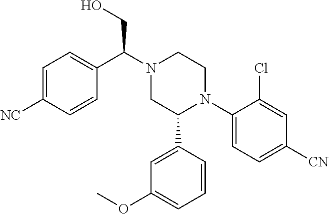 [SMILES: COc1cccc([C@@H]2CN([C@H](CO)c3ccc(C#N)cc3)CCN2c2ccc(C#N)cc2Cl)c1] |
| 852 | 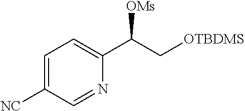 [SMILES: CC(C)(C)[Si](C)(C)OC[C@H](OS(C)(=O)=O)c1ccc(C#N)cn1] | 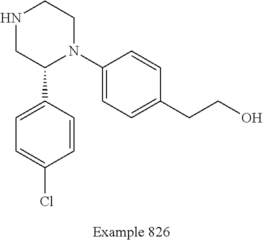 [SMILES: OCCc1ccc(N2CCNC[C@H]2c2ccc(Cl)cc2)cc1] | 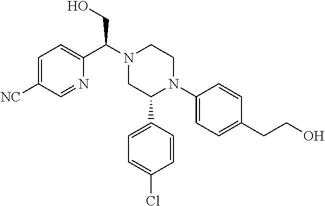 [SMILES: N#Cc1ccc([C@@H](CO)N2CCN(c3ccc(CCO)cc3)[C@H](c3ccc(Cl)cc3)C2)nc1] |
| 853 | 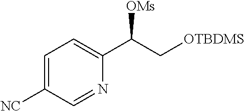 [SMILES: CC(C)(C)[Si](C)(C)OC[C@H](OS(C)(=O)=O)c1ccc(C#N)cn1] | 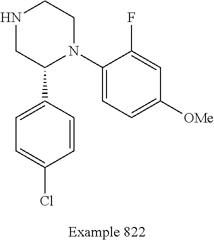 [SMILES: COc1ccc(N2CCNC[C@H]2c2ccc(Cl)cc2)c(F)c1] | 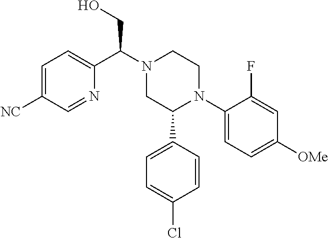 [SMILES: COc1ccc(N2CCN([C@H](CO)c3ccc(C#N)cn3)C[C@H]2c2ccc(Cl)cc2)c(F)c1] |
| 854 | 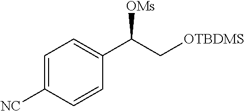 [SMILES: CC(C)(C)[Si](C)(C)OC[C@H](OS(C)(=O)=O)c1ccc(C#N)cc1] | 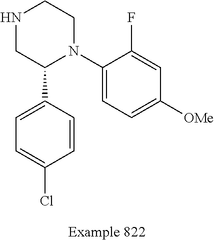 [SMILES: COc1ccc(N2CCNC[C@H]2c2ccc(Cl)cc2)c(F)c1] | 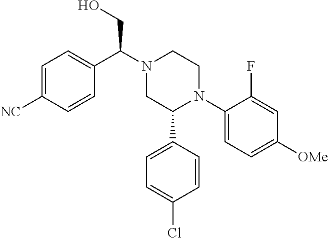 [SMILES: COc1ccc(N2CCN([C@H](CO)c3ccc(C#N)cc3)C[C@H]2c2ccc(Cl)cc2)c(F)c1] |
| 855 | 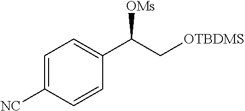 [SMILES: CC(C)(C)[Si](C)(C)OC[C@H](OS(C)(=O)=O)c1ccc(C#N)cc1] | 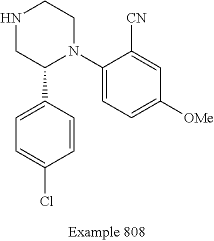 [SMILES: COc1ccc(N2CCNC[C@H]2c2ccc(Cl)cc2)c(C#N)c1] | 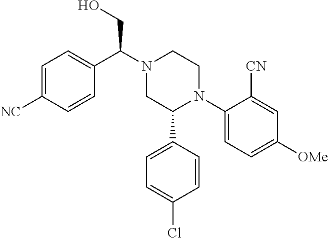 [SMILES: COc1ccc(N2CCN([C@H](CO)c3ccc(C#N)cc3)C[C@H]2c2ccc(Cl)cc2)c(C#N)c1] |
| 856 | 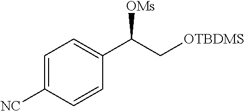 [SMILES: CC(C)(C)[Si](C)(C)OC[C@H](OS(C)(=O)=O)c1ccc(C#N)cc1] | 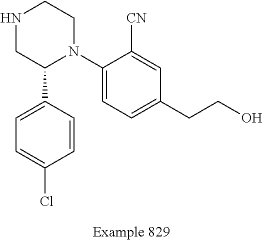 [SMILES: N#Cc1cc(CCO)ccc1N1CCNC[C@H]1c1ccc(Cl)cc1] | 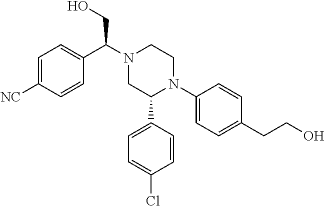 [SMILES: N#Cc1ccc([C@@H](CO)N2CCN(c3ccc(CCO)cc3)[C@H](c3ccc(Cl)cc3)C2)cc1] |
| 857 | 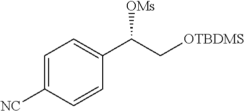 [SMILES: CC(C)(C)[Si](C)(C)OC[C@@H](OS(C)(=O)=O)c1ccc(C#N)cc1] | 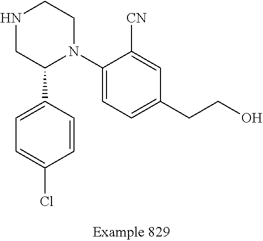 [SMILES: N#Cc1cc(CCO)ccc1N1CCNC[C@H]1c1ccc(Cl)cc1] | 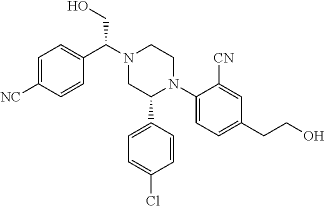 [SMILES: N#Cc1ccc([C@H](CO)N2CCN(c3ccc(CCO)cc3C#N)[C@H](c3ccc(Cl)cc3)C2)cc1] |
| 857a | 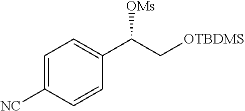 [SMILES: CC(C)(C)[Si](C)(C)OC[C@@H](OS(C)(=O)=O)c1ccc(C#N)cc1] | 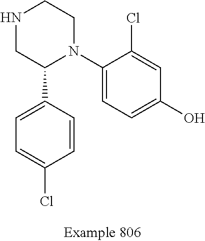 [SMILES: Oc1ccc(N2CCNC[C@H]2c2ccc(Cl)cc2)c(Cl)c1] | 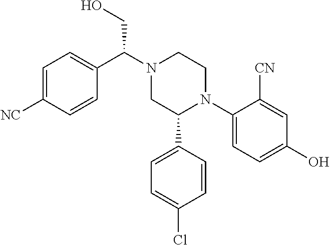 [SMILES: N#Cc1ccc([C@H](CO)N2CCN(c3ccc(O)cc3C#N)[C@H](c3ccc(Cl)cc3)C2)cc1] |

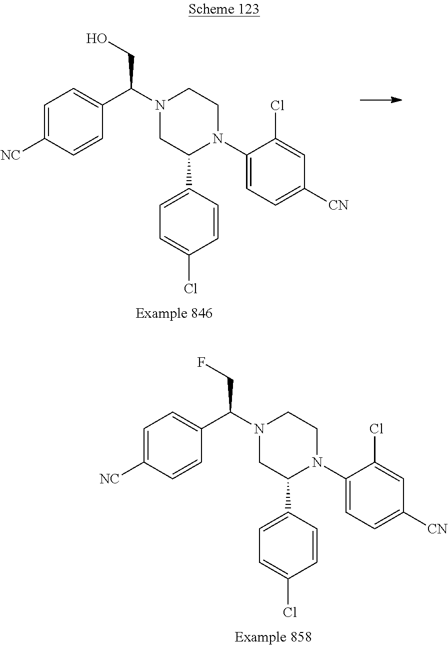 [SMILES: N#Cc1ccc([C@@H](CO)N2CCN(c3ccc(C#N)cc3Cl)[C@H](c3ccc(Cl)cc3)C2)cc1 ; N#Cc1ccc([C@@H](CF)N2CCN(c3ccc(C#N)cc3Cl)[C@H](c3ccc(Cl)cc3)C2)cc1]

To Example 846 (0.14 g, 0.30 mmol) methylene chloride (1 mL) at 0 C was added Deoxo-fluor [bis-(2-methoxyethyl) aminosulfur trifluoride] (0.1 mL). Stirred for 2.5 h allowing the cold bath to expire and then added saturated NaHCO₃. The mixture was then extracted with methylene chloride. The organic layer was washed with water and brine, dried (MgSO₄), filtered, and concentrated in vacuo The residue was purified by silica gel chromatography to provide Example 858 (0.12 g, 83%).

### Preparation of Example 859

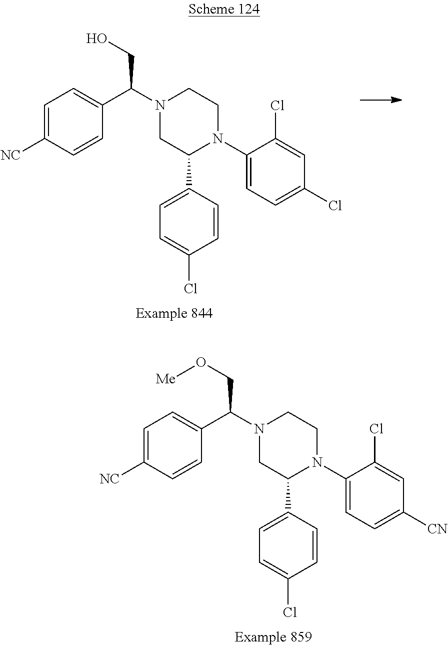 [SMILES: N#Cc1ccc([C@@H](CO)N2CCN(c3ccc(Cl)cc3Cl)[C@H](c3ccc(Cl)cc3)C2)cc1 ; COC[C@H](c1ccc(C#N)cc1)N1CCN(c2ccc(C#N)cc2Cl)[C@H](c2ccc(Cl)cc2)C1]

Example 859 was prepared from Example 844 using conditions described in Scheme 77, step 6.

### Preparation of Examples 860 and 861

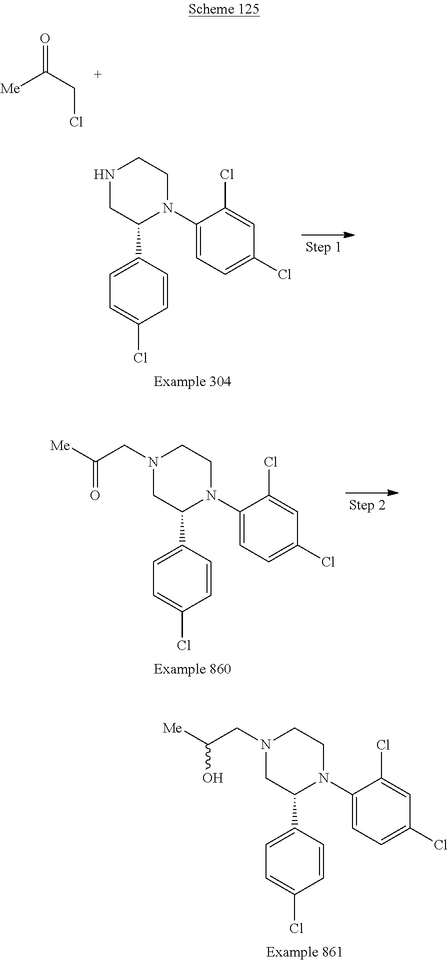

### Step 1

To Example 304 (1.0 g, 2.9 mmol) in acetonitrile (10 mL) at room temperature was added potassium carbonate (0.96 g, 7.0 mmol), sodium iodide (0.13 g, 0.9 mmol), and chloroacetone (0.32 g, 3.5 mmol). Stirred for 2 h and then added water. Extracted with EtOAc. Combined the organics and washed with water and brine. Dried (MgSO₄), filtered, and concentrated in vacuo. The residue was purified by silica gel chromatography (75-95% EtOAc/hex over 30 minutes to provide Example 860 (1.0 g, 86%).

Step 2

To Example 860 (0.10 g, 0.25 mmol) in MeOH (1 mL) at room temperature was added sodium borohydride (0.01 g, 0.25 mmol) [Gas Evolution]. Stirred for 1 h and then concentrated in vacuo. Added 2N LiOH and extracted with EtOAc. Combined the organics and washed with water and brine. Dried (MgSO₄), filtered and concentrated in vacuo. The residue was purified by silica gel chromatography (0-4% MeOH/EtOAc) to provide Example 861 (0.06 g, 60%).

### Preparation of Example 862

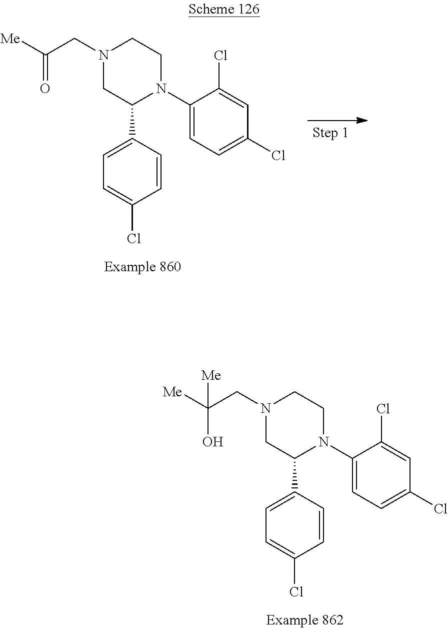 [SMILES: CC(=O)CN1CCN(c2ccc(Cl)cc2Cl)[C@H](c2ccc(Cl)cc2)C1 ; CC(C)(O)CN1CCN(c2ccc(Cl)cc2Cl)[C@H](c2ccc(Cl)cc2)C1]

To Example 860 (0.16 g, 0.42 mmol) in THF (1.5 mL) was at −78 C was added methyl magnesium bromide (3.0 M in THF, 0.17 mL, 0.52 mol). After 1 h, the cold bath was removed and the reaction was allowed to warm to room temperature. Saturated aqueous Rochelle's salt was then added and the mixture was extracted with EtOAc. The organic layer was washed with water and brine, dried (MgSO₄), filtered, and concentrated in vacuo. The residue was purified by silica gel chromatography (0-40% EtOAc/hex over 30 minutes) to provide Example 862 (0.07 g, 0.18 mmol).

### Preparation of Example 863

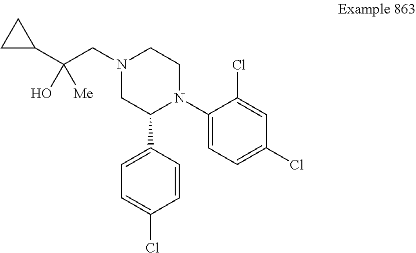 [SMILES: CC(O)(CN1CCN(c2ccc(Cl)cc2Cl)[C@H](c2ccc(Cl)cc2)C1)C1CC1]

Example 863 prepared in a similar manner as Example 862 in Scheme 126 except that cyclopropyl magnesium bromide was used instead of methyl magnesium bromide.

### Preparation of Example 864

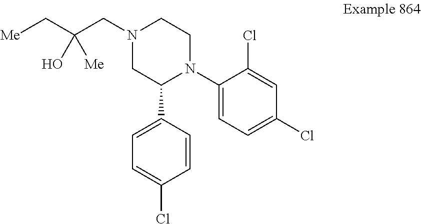 [SMILES: CCC(C)(O)CN1CCN(c2ccc(Cl)cc2Cl)[C@H](c2ccc(Cl)cc2)C1]

Example 864 prepared in a similar manner as Example 862 in Scheme 126 except that ethyl magnesium bromide was used instead of methyl magnesium bromide.

### Preparation of Example 865

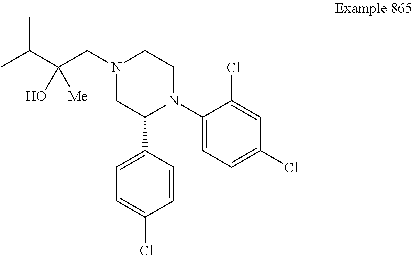 [SMILES: CC(C)C(C)(O)CN1CCN(c2ccc(Cl)cc2Cl)[C@H](c2ccc(Cl)cc2)C1]

Example 865 prepared in a similar manner as Example 862 in Scheme 126 except that isopropyl magnesium chloride was used instead of methyl magnesium bromide.

### Preparation of Example 866

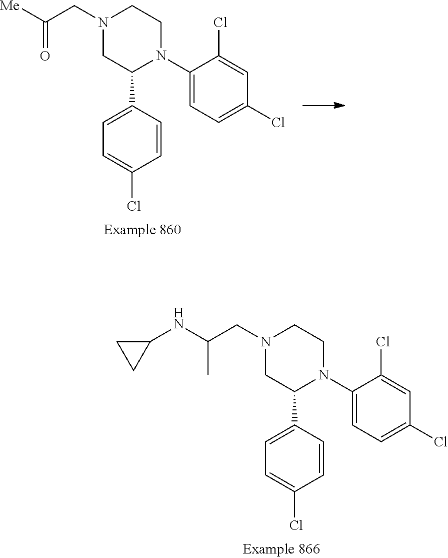 [SMILES: CC(=O)CN1CCN(c2ccc(Cl)cc2Cl)[C@H](c2ccc(Cl)cc2)C1 ; CC(CN1CCN(c2ccc(Cl)cc2Cl)[C@H](c2ccc(Cl)cc2)C1)NC1CC1]

To Example 860 (0.13 g, 0.33 mmol) in DCE (1 mL) was added cyclopropyl amine (0.03 mL, 0.41 mmol) and titanium isopropoxide (0.12 mL, 0.41 mmol). Stirred for 19 h and then added sodium cyanoborohydride (0.03 g, 0.5 mmol). Stirred for another 6 h and then added saturated NaHCO₃. Extracted the mixture with EtOAc The organic layer was washed with water and brine, dried (MgSO₄), filtered, and concentrated in vacuo. The residue was purified by preparative thin layer chromatography (2000 μm SiO₂-8% MeOH/EtOAc) to provide Example 866 (0.03 g) as a mixture of diastereomers.

### Preparation of Example 867

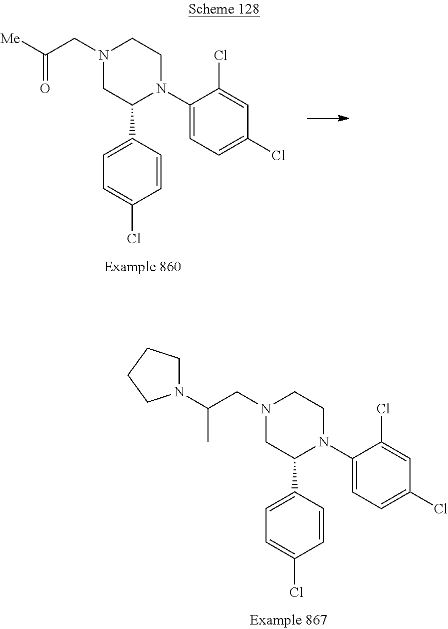 [SMILES: CC(=O)CN1CCN(c2ccc(Cl)cc2Cl)[C@H](c2ccc(Cl)cc2)C1 ; CC(CN1CCN(c2ccc(Cl)cc2Cl)[C@H](c2ccc(Cl)cc2)C1)N1CCCC1]

To Example 860 (0.17 g, 0.42 mmol) in DCE was added pyrrolidine (0.06 g, 0.84 mmol) sodium triacetoxyborohydride (0.18 g, 0.84 mmol) followed by a drop of acetic acid. The reaction was stirred for 16 h and then DCM was added. The mixture was washed with saturated NaHCO₃, water and brine. The organic layer was washed water and brine, dried (MgSO₄), filtered, and concentrated in vacuo. The residue was purified by silica gel chromatography (0-8% 7N NH₃ in MeOH/DCM) to provide Example 867 (0.13 g).

### Preparation of Example 868

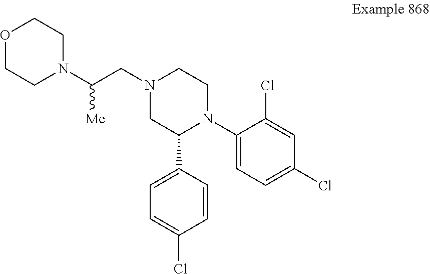 [SMILES: CC(CN1CCN(c2ccc(Cl)cc2Cl)[C@H](c2ccc(Cl)cc2)C1)N1CCOCC1]

Example 868 was prepared in a same manner as Example 867 in scheme 128 except that morpholine was used instead of pyrrolidine.

### Preparation of Examples 868c

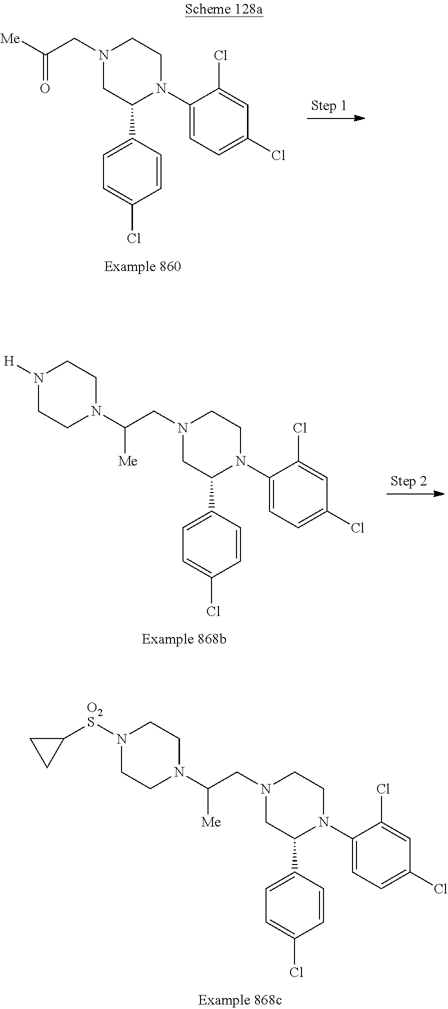 [SMILES: CC(=O)CN1CCN(c2ccc(Cl)cc2Cl)[C@H](c2ccc(Cl)cc2)C1 ; CC(CN1CCN(c2ccc(Cl)cc2Cl)[C@H](c2ccc(Cl)cc2)C1)N1CCNCC1 ; CC(CN1CCN(c2ccc(Cl)cc2Cl)[C@H](c2ccc(Cl)cc2)C1)N1CCN(S(=O)(=O)C2CC2)CC1]

### Step 1

To Example 860 (0.20 g, 0.51 mmol) in dichloroethane (2 mL) was added N-Boc-piperidine (0.14 g, 0.77 mmol), sodium triacetoxyborohydride (0.16 g, 0.77 mL), and a drop of acetic acid. The mixture was stirred at room temperature for 16 h. An additional 0.5 equivs of N-Boc-piperidine and sodium triacetoxy borohydride was added and the mixture was stirred for an additional 72 h. The reaction mixture was then diluted with dichloromethane and washed with saturated NaHCO₃, water, and brine. The organic layer was dried (MgSO₄), filtered, and concentrated in vacuo. The residue was purified by silica gel chromatography eluting with 25-100% EtOAc/hexanes over 30 minutes. The resulting residue was taken up into dichloromethane (2 mL) and 2N HCl/ether (1 mL) was added. The reaction was stirred at room temperature for 16 h and then concentrated in vacuo to provide Example 868b (0.070 g) as the HCl salt.

### Step 2

To Example 868b (0.070 g, 0.13 mmol) in dichloromethane (1 mL) was added triethylamine (0.06 mL, 0.46 mmol) and cyclopropylsulfonyl chloride (0.037 g, 0.26 mmol). The reaction was stirred at room temperature for 16 h. The mixture was then diluted with dichloromethane and washed with saturated NaHCO₃, water, and brine. The organic layer was dried (MgSO₄), filtered, and concentrated in vacuo. The residue was purified by silica gel chromatography eluting with 0-5% MeOH/EtOAc over 30 minutes to provide Example 868c.

### Preparation of Examples 869 and 870

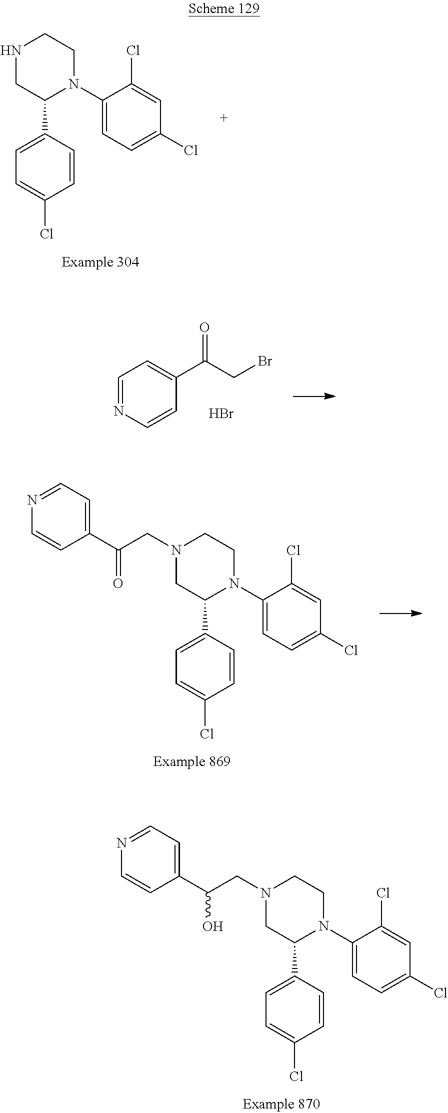 [SMILES: Clc1ccc([C@@H]2CNCCN2c2ccc(Cl)cc2Cl)cc1 ; O=C(CBr)c1ccncc1 ; Br ; O=C(CN1CCN(c2ccc(Cl)cc2Cl)[C@H](c2ccc(Cl)cc2)C1)c1ccncc1 ; OC(CN1CCN(c2ccc(Cl)cc2Cl)[C@H](c2ccc(Cl)cc2)C1)c1ccncc1]

### Step 1

To Example 304 in THF (5 mL) at room temperature was added diisopropylethylamine and 4-(bromoacetyl)pyridine hydrobromide. After 1 h added DCM and washed the mixture with saturated NaHCO₃, water, and brine. The organic layer was dried (MgSO₄), filtered, and concentrated in vacuo. The residue was purified by silica gel chromatography (0-2% MeOH/EtOAc) to provide Example 869 (0.265 g).

### Step 2

To Example 869 (0.26 g, 0.57 mmol) in MeOH (0 C) was added sodium borohydride (0.022 g, 0.57 mmol) [Gas Evolution]. The reaction was stirred for 19 h and then concentrated in vacuo. To the residue was added 2N LiOH and EtOAc. The mixture was stirred vigorously for 10 minutes. The mixture was extracted with EtOAc. The organic layer was washed with water and brine, dried (MgSO₄), filtered, and concentrated in vacuo. The residue was purified by silica gel chromatography (0-5% MeOH/EtOAc over 30 minutes to provide Example 870 (0.23 g).

### Preparation of Examples 871-873

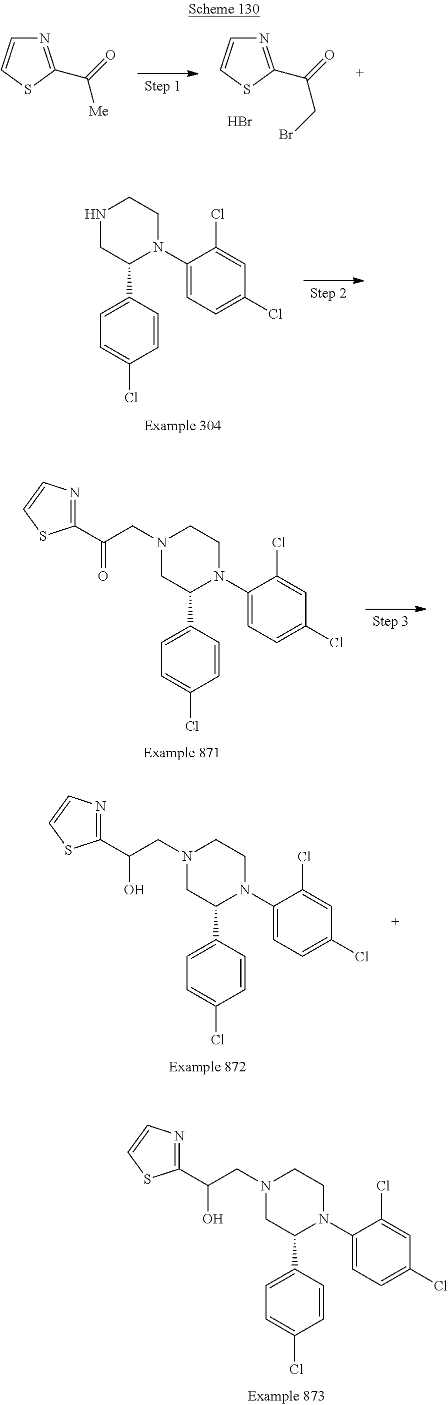

### Step 1

To 2-acetylthiazole (1.0 g, 7.9 mmol) in acetic acid (26 mL) was added bromine (0.48 mL, 9.4 mmol). Warmed the reaction to reflux and stirred for ˜40 minutes. Cooled to room temperature and filtered off the resultant yellow solid. Washed the solid with ether and air-dried to provide the α-bromoketone (2.35 g) that was contaminated with some of the dibromoketone. The material was used directly without further purification.

### Step 2

Example 304 (0.56 g, 1.5 mmol) was treated with the bromoketone prepared in step 1 using the conditions described in Scheme X step 1 to provide Example 871 (0.56 g, 1.2 mmol).

### Step 3

To Example 871 (0.56 g, 1.2 mmol) in MeOH (4 mL) at 0 C was added sodium borohydride (0.05 g, 1.2 mmol) [Gas Evolution]. Took the cold bath away and stirred for 3 h. Water was added and the mixture was extracted with EtOAc. The organic layer was washed with water and brine, dried (MgSO₄), filtered, and concentrated in vacuo. The residue was purified by silica gel chromatography (0-80% EtOAc/hex over 30 minutes) to provide the hydroxy product as a mixture of diastereomers (0.45 g, 0.96 mmol). A sample of the mixture of diastereomers was separated by HPLC [Chiralcel OD, 90% hex/IPA, 50 ml/min, 254 nm] to provide Example 872 and Example 873.

### Preparation of Example 874

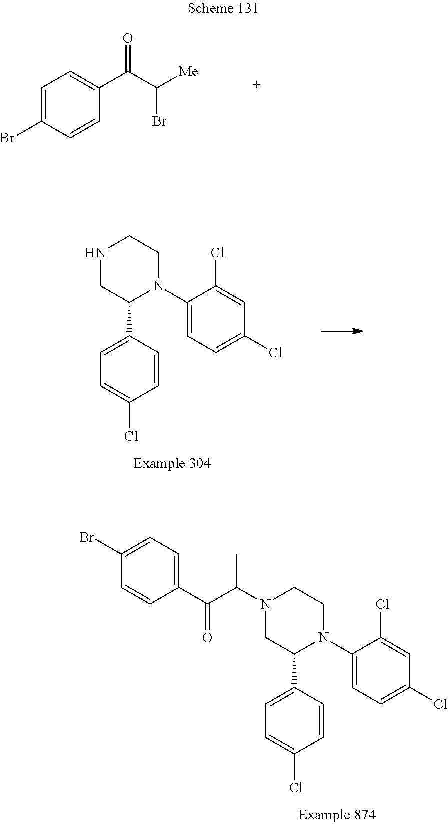 [SMILES: CC(Br)C(=O)c1ccc(Br)cc1 ; Clc1ccc([C@@H]2CNCCN2c2ccc(Cl)cc2Cl)cc1 ; CC(C(=O)c1ccc(Br)cc1)N1CCN(c2ccc(Cl)cc2Cl)[C@H](c2ccc(Cl)cc2)C1]

Example 874 was prepared in the same manner as described in Scheme 69 except that 2,4′-dibromopropiophenone was used instead of 2-bromoacetophenone.

### Preparation of Example 875

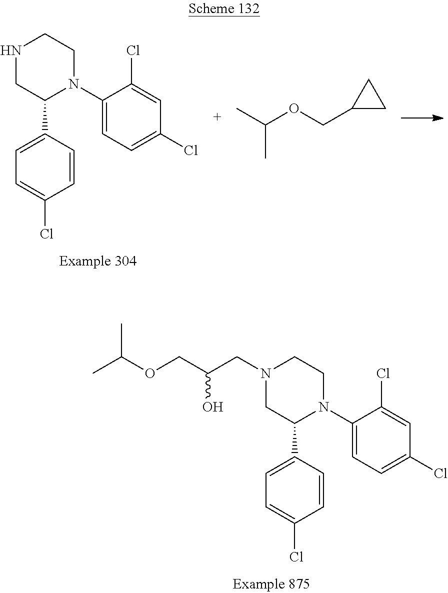

To Example 304 (0.14 g, 0.41 mmol) was added the isopropyl glycidyl ether (0.06 g, 0.51 mmol). Warmed the reaction to 100 C and stirred for 20 h. Cooled to room temperature and purified the reaction mixture directly by silica gel chromatography (0-100% EtOAc/hex over 30 minutes) to provide Example 875 (0.16 g, 0.35 mmol).

### Preparation of Example 876

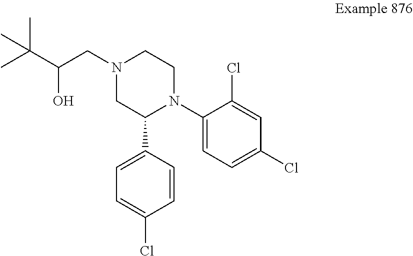 [SMILES: CC(C)(C)C(O)CN1CCN(c2ccc(Cl)cc2Cl)[C@H](c2ccc(Cl)cc2)C1]

Example 876 was prepared in the same manner as Example 875 except that 3,3-dimethyl-1,2-epoxybutane was used instead of isopropyl glycidyl ether.

### Preparation of Examples 877a, 877b, 878

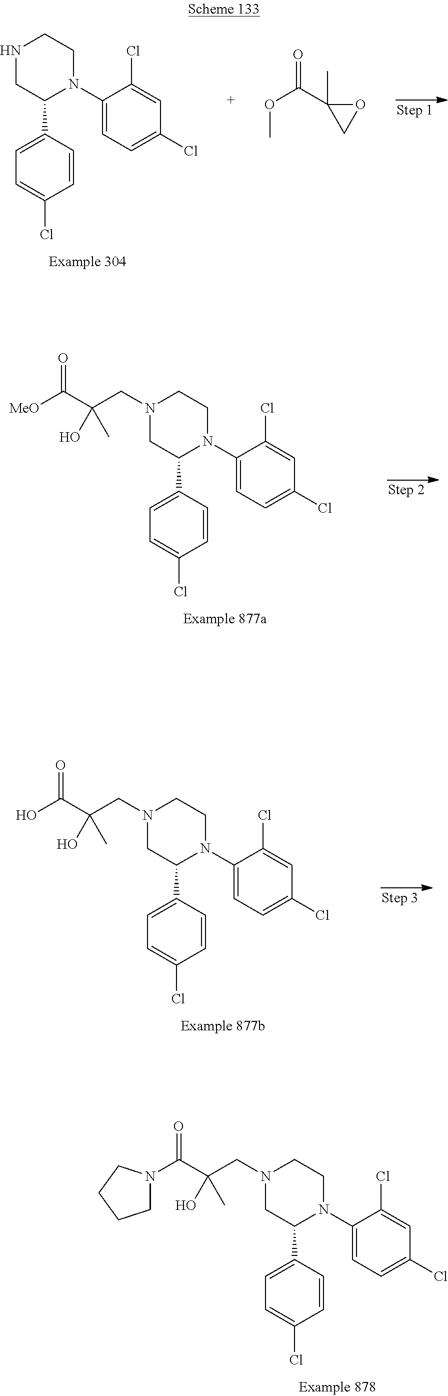

### Step 1

To Example 304 (1.5 g, 4.4 mmol) in a pressure tube was added methyl-2-methylglycidate (0.51 mL, 4.8 mmol). The tube was sealed and the reaction stirred at 100 C for 18 h. The reaction mixture was cooled to room temperature and purified directly by silica gel chromatography (0-30% EtOAc/hex over 30 minutes) to provide Example 877a (2.0 g, 4.4 mmol).

### Step 2

To Example 877a (0.27 g, 0.6 mmol) in MeOH (2 mL) was added LiOH_{aq}(2N, 0.9 mL, 1.8 mmol). The reaction was stirred at room temperature for 18 h and then concentrated in vacuo. To the residue was added 1N HCl to obtain a pH —5. The mixture was extracted with EtOAc. The organic layer was washed with water and brine, dried (MgSO₄), filtered, and concentrated in vacou to provide Example 877b (0.25 g, 0.56 mmol).

### Step 3

To Example 877b (0.10 g, 0.22 mmol) in DCE (0.9 mL) was added pyrrolidine (0.03 mL, 0.34 mmol), HOBt (0.014 g, 0.11 mmol), and EDCI (0.065 g, 0.34 mmol). The reaction was stirred for 18 h and then diluted with DCM. The mixture was washed with 2N LiOH, water, and brine. The organic layer was dried (MgSO₄), filtered, and concentrated in vacuo. The residue was purified by silica gel chromatography (0-70% EtOAc/hex) to provide Example 878 (0.080 g, 0.16 mmol).

### Preparation of Example 879

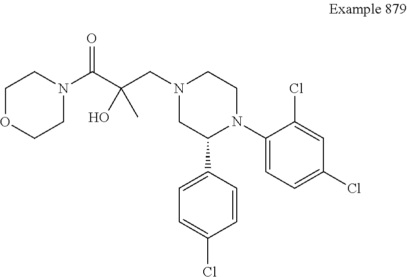 [SMILES: CC(O)(CN1CCN(c2ccc(Cl)cc2Cl)[C@H](c2ccc(Cl)cc2)C1)C(=O)N1CCOCC1]

Example 879 was prepared in a similar fashion as Example 878 in scheme 133 except that morpholine was used instead of pyrrolidine in step 3.

### Preparation of Examples 880-882

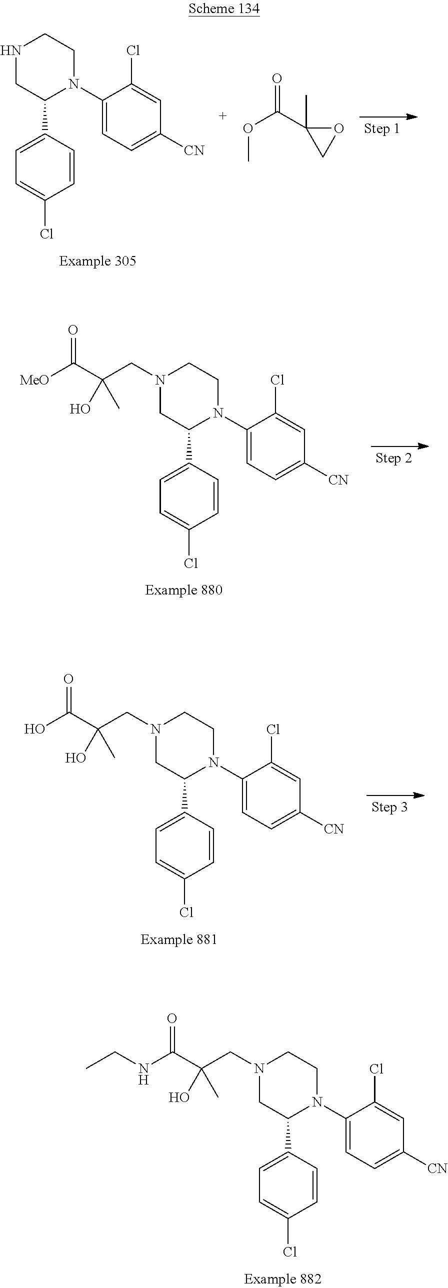

Examples 880 and 881 were prepared in a similar fashion as described in Scheme 133 except that Example 305 was used instead of Example 304 in step 1.

Example 881 was converted to Example 882 by using the conditions in Scheme 133 step 3 except that ethyl amine was used instead of pyrrolidine.

### Preparation of Example 883

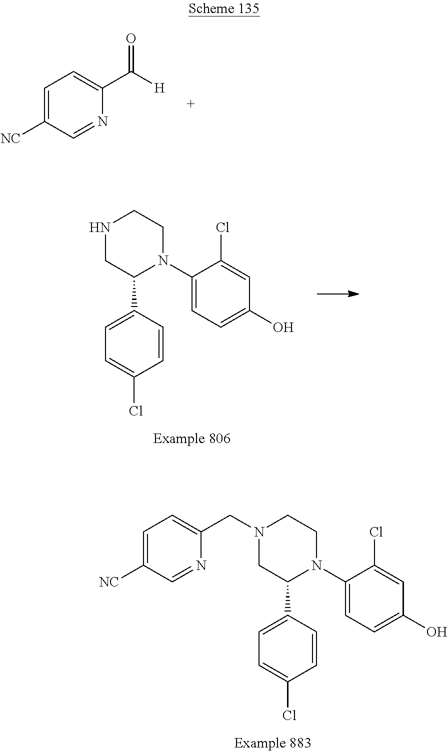 [SMILES: N#Cc1ccc(C=O)nc1 ; Oc1ccc(N2CCNC[C@H]2c2ccc(Cl)cc2)c(Cl)c1 ; N#Cc1ccc(CN2CCN(c3ccc(O)cc3Cl)[C@H](c3ccc(Cl)cc3)C2)nc1]

To Example 806 (0.06 g, 0.18 mmol) in DCE (1 mL) was added 2-formyl-5-cyanopyridine (0.03 g, 0.23 mmol) [prepared in Scheme 106] and sodium triacetoxyborohydride (0.06 g, 0.28 mmol). The reaction was stirred at room temperature for 16 h and then diluted with DCM. The mixture was washed with saturated NaHCO₃, water, and brine. The organic layer was dried (MgSO₄), filtered, and concentrated in vacuo. The residue was purified by silica gel chromatography (0-60% EtOAc/hex over 30 minutes) to provide Example 883 (0.07 g, 0.16 mmol).

### Preparation of Example 884

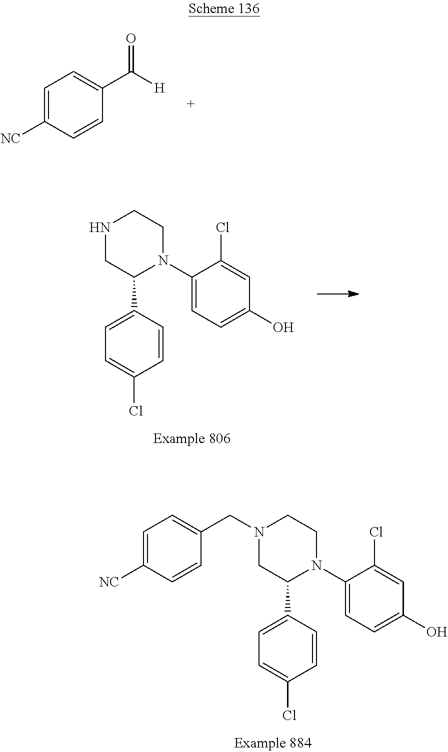 [SMILES: N#Cc1ccc(C=O)cc1 ; Oc1ccc(N2CCNC[C@H]2c2ccc(Cl)cc2)c(Cl)c1 ; N#Cc1ccc(CN2CCN(c3ccc(O)cc3Cl)[C@H](c3ccc(Cl)cc3)C2)cc1]

Example 884 was prepared in the same manner as Example 883 in scheme 135 (above) except that 4-cyanobenzaldehyde was used instead of 2-formyl-5-cyanopyridine.

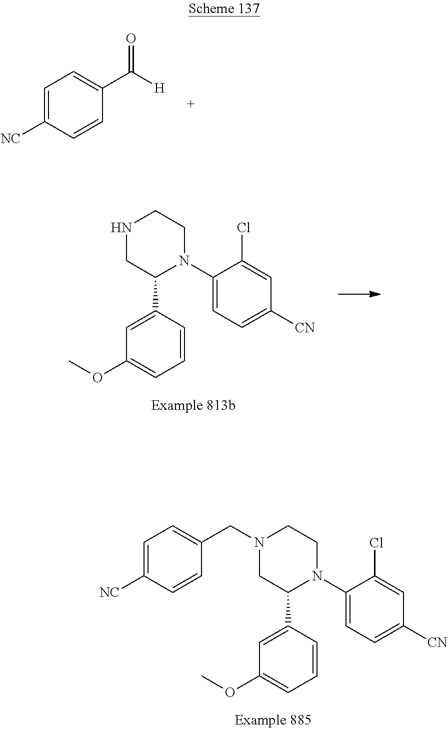 [SMILES: N#Cc1ccc(C=O)cc1 ; COc1cccc([C@@H]2CNCCN2c2ccc(C#N)cc2Cl)c1 ; COc1cccc([C@@H]2CN(Cc3ccc(C#N)cc3)CCN2c2ccc(C#N)cc2Cl)c1]

Example 885 was prepared in the same manner as Example 883 in Scheme 136 (above) except that 4-cyanobenzaldehyde and the methoxy-piperazine Ex. 813b were used.

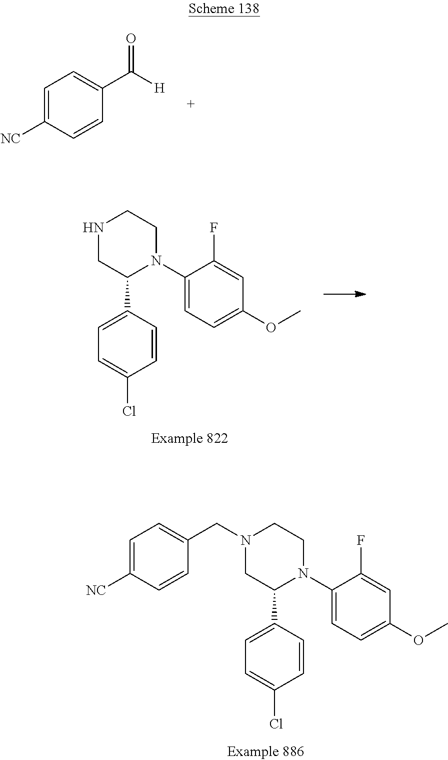 [SMILES: N#Cc1ccc(C=O)cc1 ; COc1ccc(N2CCNC[C@H]2c2ccc(Cl)cc2)c(F)c1 ; COc1ccc(N2CCN(Cc3ccc(C#N)cc3)C[C@H]2c2ccc(Cl)cc2)c(F)c1]

Example 886 was prepared in the same manner as Example 883 in Scheme 135 (above) except that 4-cyanobenzaldehyde and the piperazine Ex. 822 were used.

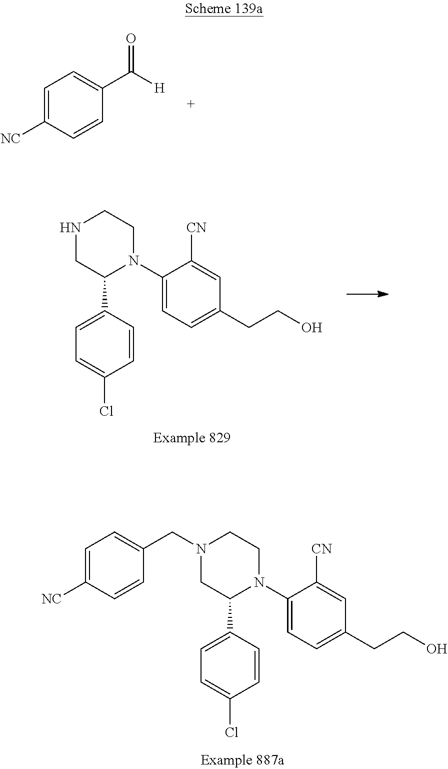 [SMILES: N#Cc1ccc(C=O)cc1 ; N#Cc1cc(CCO)ccc1N1CCNC[C@H]1c1ccc(Cl)cc1 ; N#Cc1ccc(CN2CCN(c3ccc(CCO)cc3C#N)[C@H](c3ccc(Cl)cc3)C2)cc1]

Example 887a was prepared in the same manner as Example 883 in Scheme 135 (above) except that 4-cyanobenzaldehyde and the piperazine Ex. 829 were used.

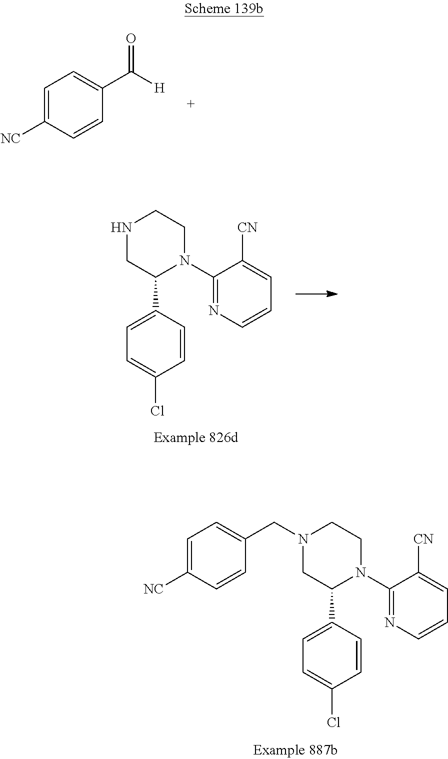 [SMILES: N#Cc1ccc(C=O)cc1 ; N#Cc1cccnc1N1CCNC[C@H]1c1ccc(Cl)cc1 ; N#Cc1ccc(CN2CCN(c3ncccc3C#N)[C@H](c3ccc(Cl)cc3)C2)cc1]

Example 887b was prepared in the same manner as Example 883 in Scheme 135 (above) except that 4-cyanobenzaldehyde and the piperazine Ex. 826d were used.

### Preparation of Example 888

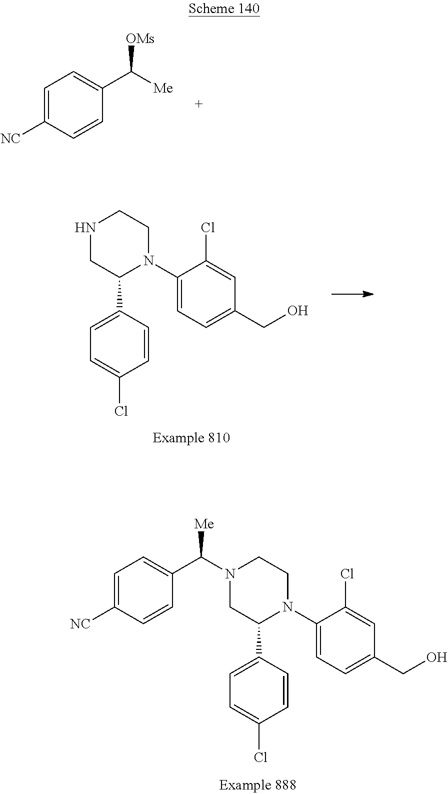 [SMILES: C[C@H](OS(C)(=O)=O)c1ccc(C#N)cc1 ; OCc1ccc(N2CCNC[C@H]2c2ccc(Cl)cc2)c(Cl)c1 ; C[C@H](c1ccc(C#N)cc1)N1CCN(c2ccc(CO)cc2Cl)[C@H](c2ccc(Cl)cc2)C1]

Example 888 was prepared in a similar manner as Example 392a in Scheme 66.

### Preparation of Examples 889-899

Examples 889-899 were prepared according to the conditions in Scheme 66, step 3, using the mesylate [prepared in step 1 and step 2 of Scheme 66] and the appropriate piperazine core. In most cases, the final target contained ˜10% of the other diastereomer.

TABLE XXXIV

| Exam- | | | |
|---|---|---|---|
| ple | | | |
| # | Mesylate | Piperazine | Example Structure |
| 889 | 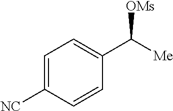 [SMILES: C[C@H](OS(C)(=O)=O)c1ccc(C#N)cc1] | 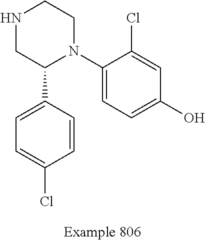 [SMILES: Oc1ccc(N2CCNC[C@H]2c2ccc(Cl)cc2)c(Cl)c1] | 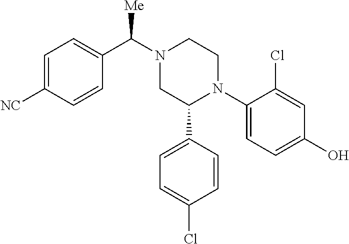 [SMILES: C[C@H](c1ccc(C#N)cc1)N1CCN(c2ccc(O)cc2Cl)[C@H](c2ccc(Cl)cc2)C1] |
| 890 | 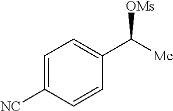 [SMILES: C[C@H](OS(C)(=O)=O)c1ccc(C#N)cc1] | 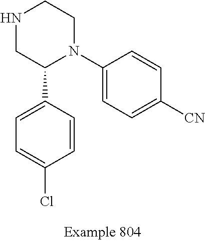 [SMILES: N#Cc1ccc(N2CCNC[C@H]2c2ccc(Cl)cc2)cc1] | 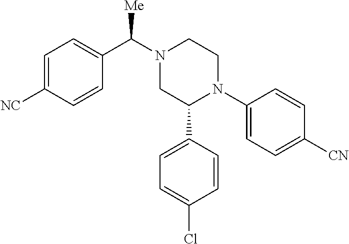 [SMILES: C[C@H](c1ccc(C#N)cc1)N1CCN(c2ccc(C#N)cc2)[C@H](c2ccc(Cl)cc2)C1] |
| 891 | 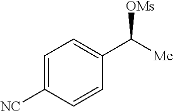 [SMILES: C[C@H](OS(C)(=O)=O)c1ccc(C#N)cc1] | 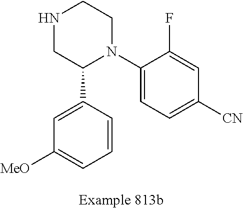 [SMILES: COc1cccc([C@@H]2CNCCN2c2ccc(C#N)cc2F)c1] | 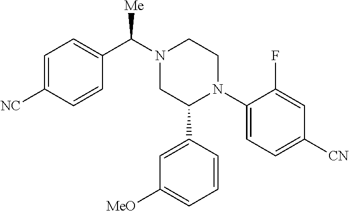 [SMILES: COc1cccc([C@@H]2CN([C@H](C)c3ccc(C#N)cc3)CCN2c2ccc(C#N)cc2F)c1] |
| 892 | 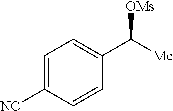 [SMILES: C[C@H](OS(C)(=O)=O)c1ccc(C#N)cc1] | 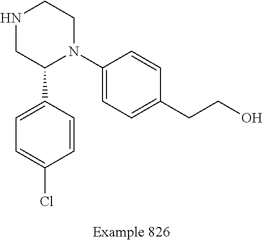 [SMILES: OCCc1ccc(N2CCNC[C@H]2c2ccc(Cl)cc2)cc1] | 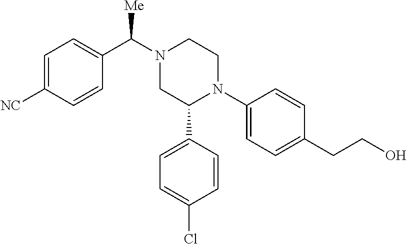 [SMILES: C[C@H](c1ccc(C#N)cc1)N1CCN(c2ccc(CCO)cc2)[C@H](c2ccc(Cl)cc2)C1] |
| 893 | 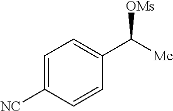 [SMILES: C[C@H](OS(C)(=O)=O)c1ccc(C#N)cc1] | 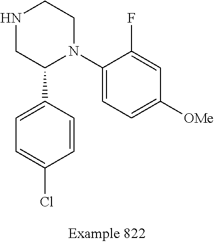 [SMILES: COc1ccc(N2CCNC[C@H]2c2ccc(Cl)cc2)c(F)c1] | 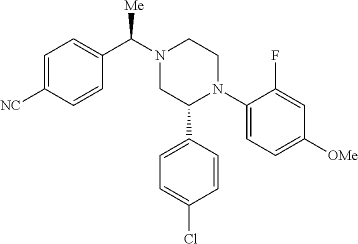 [SMILES: COc1ccc(N2CCN([C@H](C)c3ccc(C#N)cc3)C[C@H]2c2ccc(Cl)cc2)c(F)c1] |
| 894 | 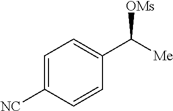 [SMILES: C[C@H](OS(C)(=O)=O)c1ccc(C#N)cc1] | 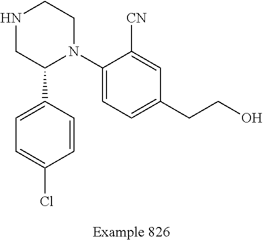 [SMILES: N#Cc1cc(CCO)ccc1N1CCNC[C@H]1c1ccc(Cl)cc1] | 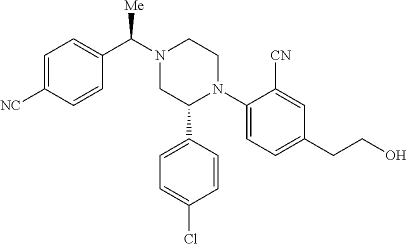 [SMILES: C[C@H](c1ccc(C#N)cc1)N1CCN(c2ccc(CCO)cc2C#N)[C@H](c2ccc(Cl)cc2)C1] |
| 895 | 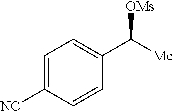 [SMILES: C[C@H](OS(C)(=O)=O)c1ccc(C#N)cc1] | 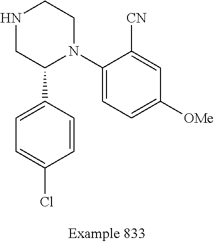 [SMILES: COc1ccc(N2CCNC[C@H]2c2ccc(Cl)cc2)c(C#N)c1] | 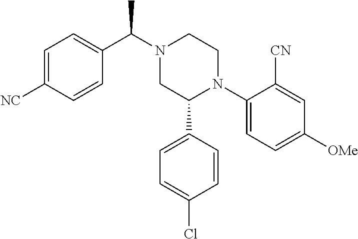 [SMILES: COc1ccc(N2CCN([C@H](C)c3ccc(C#N)cc3)C[C@H]2c2ccc(Cl)cc2)c(C#N)c1] |
| 896 | 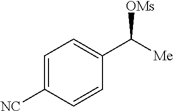 [SMILES: C[C@H](OS(C)(=O)=O)c1ccc(C#N)cc1] | 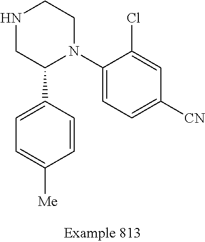 [SMILES: Cc1ccc([C@@H]2CNCCN2c2ccc(C#N)cc2Cl)cc1] | 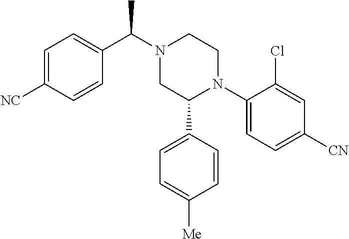 [SMILES: Cc1ccc([C@@H]2CN([C@H](C)c3ccc(C#N)cc3)CCN2c2ccc(C#N)cc2Cl)cc1] |
| 897 | 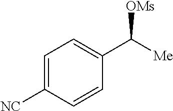 [SMILES: C[C@H](OS(C)(=O)=O)c1ccc(C#N)cc1] | 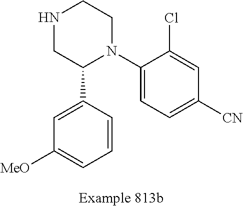 [SMILES: COc1cccc([C@@H]2CNCCN2c2ccc(C#N)cc2Cl)c1] | 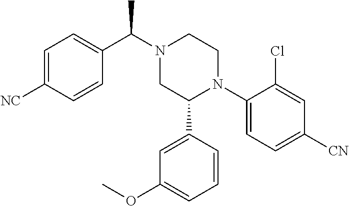 [SMILES: COc1cccc([C@@H]2CN([C@H](C)c3ccc(C#N)cc3)CCN2c2ccc(C#N)cc2Cl)c1] |
| 899b | 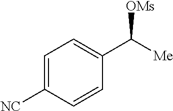 [SMILES: C[C@H](OS(C)(=O)=O)c1ccc(C#N)cc1] | 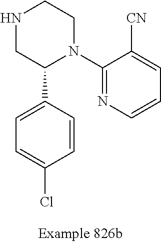 [SMILES: N#Cc1cccnc1N1CCNC[C@H]1c1ccc(Cl)cc1] | 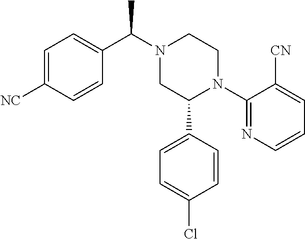 [SMILES: C[C@H](c1ccc(C#N)cc1)N1CCN(c2ncccc2C#N)[C@H](c2ccc(Cl)cc2)C1] |

### Preparation of Example 900 and Example 901

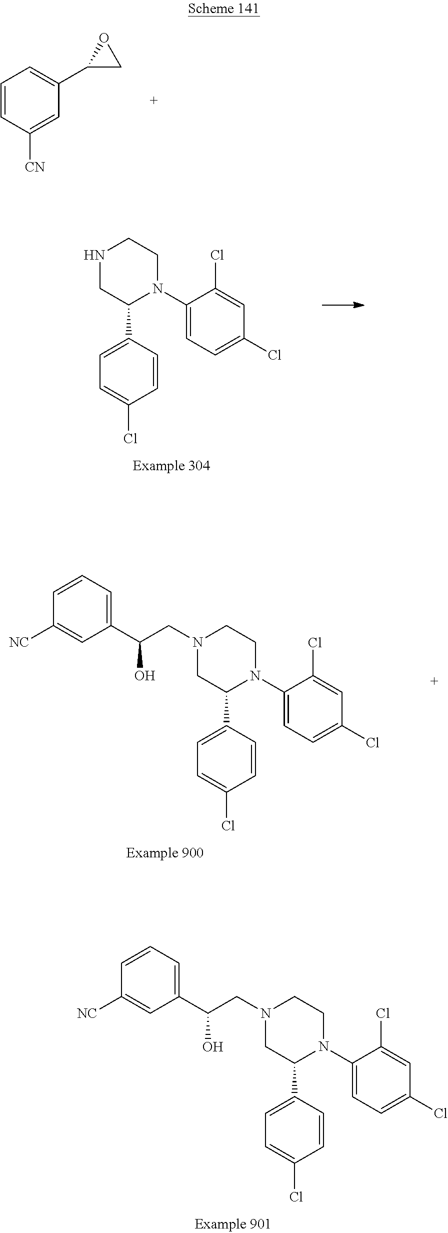 [SMILES: N#Cc1cccc([C@H]2CO2)c1 ; Clc1ccc([C@@H]2CNCCN2c2ccc(Cl)cc2Cl)cc1 ; N#Cc1cccc([C@H](O)CN2CCN(c3ccc(Cl)cc3Cl)[C@H](c3ccc(Cl)cc3)C2)c1 ; N#Cc1cccc([C@@H](O)CN2CCN(c3ccc(Cl)cc3Cl)[C@H](c3ccc(Cl)cc3)C2)c1]

To the epoxide ((0.059 g, 0.40 mmol) [prepared in a similar fashion as the epoxide in Scheme 79 except that 2-bromo-3′-cyanoacetophenone was used instead of 2-bromo-4′-cyanoacetophenone] was added Example 304 (0.125 g, 0.37 mmol). The reaction was heated neat [without solvent] to 100 C and stirred for 18 h. The reaction was cooled to room temperature and purified directly to remove the minor regioisomeric epoxide opening side product (SiO₂: 0-50% EtOAc/hex over 30 minutes) to provide the desired product as a mixture of diastereomers (0.17 g).

The diastereomers were separated by HPLC chromatography [Chiralcel OD, 80% hexane/IPA, 50 mL/min, 254 nm] to provide the desired Example 900 (0.12 g, 0.25 mmol) and the diastereomer Example 901 (0.013 g, 0.03 mmol).

### Preparation of Examples 902-904

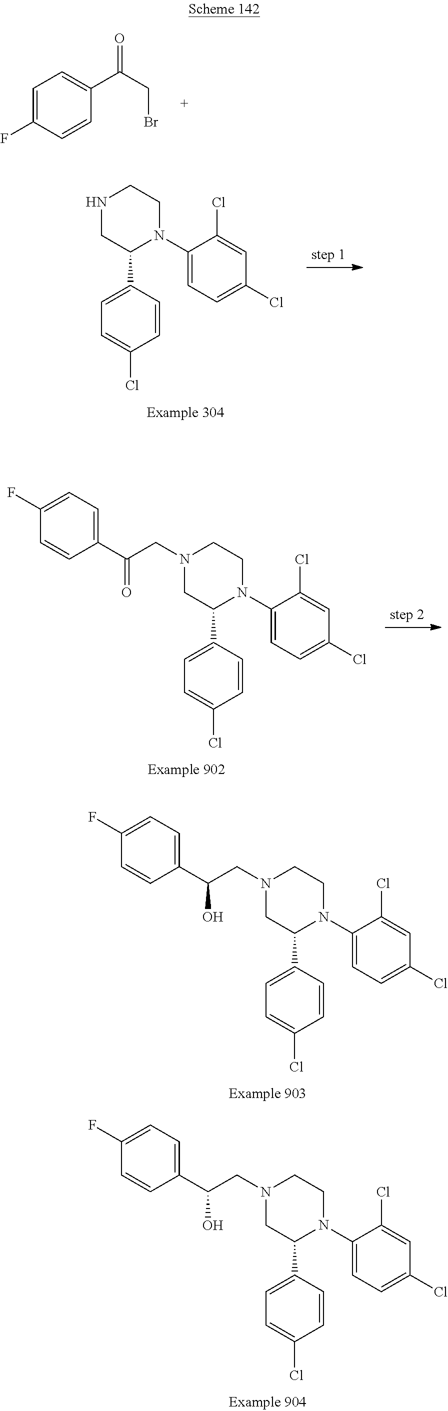 [SMILES: O=C(CBr)c1ccc(F)cc1 ; Clc1ccc([C@@H]2CNCCN2c2ccc(Cl)cc2Cl)cc1 ; O=C(CN1CCN(c2ccc(Cl)cc2Cl)[C@H](c2ccc(Cl)cc2)C1)c1ccc(F)cc1 ; O[C@H](CN1CCN(c2ccc(Cl)cc2Cl)[C@H](c2ccc(Cl)cc2)C1)c1ccc(F)cc1 ; O[C@@H](CN1CCN(c2ccc(Cl)cc2Cl)[C@H](c2ccc(Cl)cc2)C1)c1ccc(F)cc1]

### Step 1

Example 902 was prepared in a similar fashion as Example 398 in scheme 69 except that 2-bromo-4′-fluoroacetophenone was used instead of 2-bromoacetophenone. The eluent for silica gel chromatography was 35% EtOAc/hex.

### Step 2

Example 903 and Example 904 were prepared in the same manner as Example 776a and Example 776b in Scheme 78 except that Example 902 was used instead of Example 399. Also, the solvent used for the separation of Example 903 (faster eluting isomer) and Example 904 (slower eluting isomer) by chiral HPLC was 95% hexane/IPA (Chiralcel OD column).

### Preparation of Examples 905-907

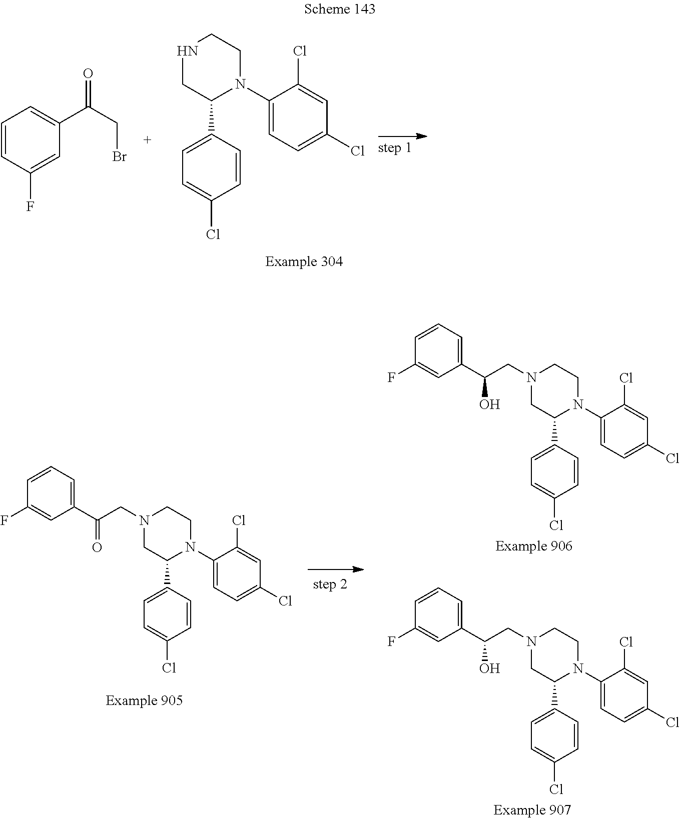 [SMILES: O=C(CBr)c1cccc(F)c1 ; Clc1ccc([C@@H]2CNCCN2c2ccc(Cl)cc2Cl)cc1 ; O=C(CN1CCN(c2ccc(Cl)cc2Cl)[C@H](c2ccc(Cl)cc2)C1)c1cccc(F)c1 ; O[C@@H](CN1CCN(c2ccc(Cl)cc2Cl)[C@H](c2ccc(Cl)cc2)C1)c1cccc(F)c1 ; O[C@H](CN1CCN(c2ccc(Cl)cc2Cl)[C@H](c2ccc(Cl)cc2)C1)c1cccc(F)c1]

### Step 1

Example 905 was prepared in a similar fashion as Example 398 in scheme 69 except that 2-bromo-3′-fluoroacetophenone was used instead of 2-bromoacetophenone. The eluent for silica gel chromatography was 35% EtOAc/hex.

### Step 2

Example 906 and Example 907 were prepared in the same manner as Example 776a and Example 776b in Scheme 78 except that Example 905 was used instead of Example 399. Also, the solvent used for the separation of Example 906 (faster eluting isomer) and Example 907 (slower eluting isomer) by chiral HPLC was 95% hexane/IPA (Chiralcel OD column).

### Preparation of Examples 908-910

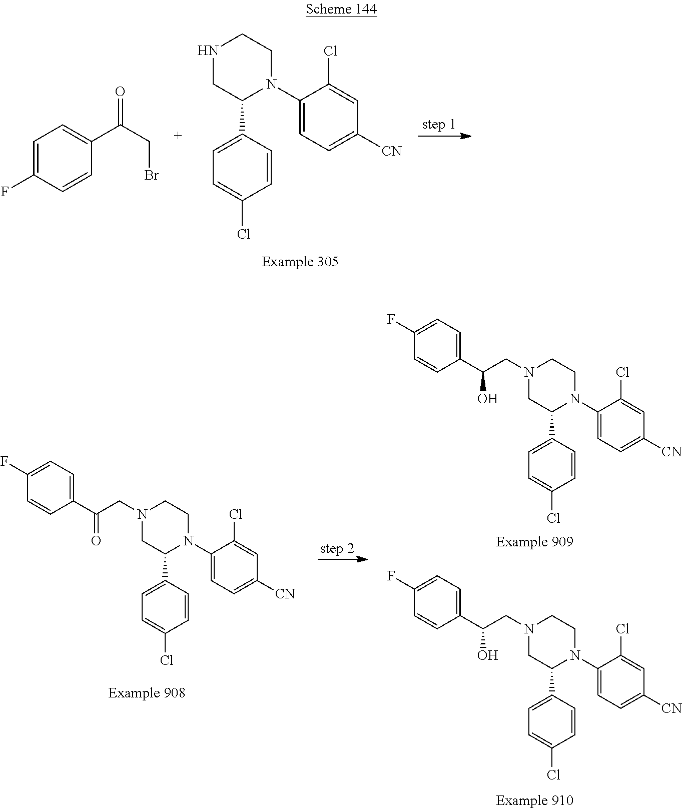 [SMILES: O=C(CBr)c1ccc(F)cc1 ; N#Cc1ccc(N2CCNC[C@H]2c2ccc(Cl)cc2)c(Cl)c1 ; N#Cc1ccc(N2CCN(CC(=O)c3ccc(F)cc3)C[C@H]2c2ccc(Cl)cc2)c(Cl)c1 ; N#Cc1ccc(N2CCN(C[C@@H](O)c3ccc(F)cc3)C[C@H]2c2ccc(Cl)cc2)c(Cl)c1 ; N#Cc1ccc(N2CCN(C[C@H](O)c3ccc(F)cc3)C[C@H]2c2ccc(Cl)cc2)c(Cl)c1]

### Step 1

Example 908 was prepared in a similar fashion as Example 398 in scheme 69 except that 2-bromo-4′-fluoroacetophenone was used instead of 2-bromoacetophenone and Example 305 was used instead of Example 304. The eluent for silica gel chromatography was 50% EtOAc/hex.

### Step 2

Example 909 and Example 910 were prepared in the same manner as Example 776a and Example 776b in Scheme 78 except that Example 908 was used instead of Example 399. Also, the solvent used for the separation of Example 909 (faster eluting isomer) and Example 910 (slower eluting isomer) by chiral HPLC was 90% hexane/IPA (Chiralcel OD column).

### Preparation of Examples 911-913

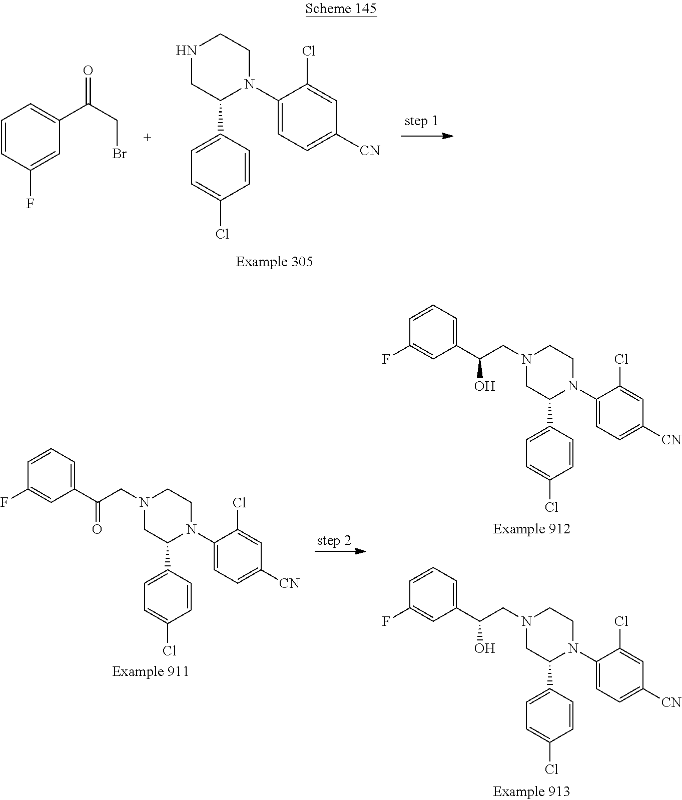 [SMILES: O=C(CBr)c1cccc(F)c1 ; N#Cc1ccc(N2CCNC[C@H]2c2ccc(Cl)cc2)c(Cl)c1 ; N#Cc1ccc(N2CCN(CC(=O)c3cccc(F)c3)C[C@H]2c2ccc(Cl)cc2)c(Cl)c1 ; N#Cc1ccc(N2CCN(C[C@@H](O)c3cccc(F)c3)C[C@H]2c2ccc(Cl)cc2)c(Cl)c1 ; N#Cc1ccc(N2CCN(C[C@H](O)c3cccc(F)c3)C[C@H]2c2ccc(Cl)cc2)c(Cl)c1]

### Step 1

Example 911 was prepared in a similar fashion as Example 398 in scheme 69 except that 2-bromo-3′-fluoroacetophenone was used instead of 2-bromoacetophenone and Example 305 was used instead of Example 304. The eluent for silica gel chromatography was 50% EtOAc/hex.

### Step 2

Example 912 and Example 913 were prepared in the same manner as Example 776a and Example 776b in Scheme 78 except that Example 911 was used instead of Example 399. Also, the solvent used for the separation of Example 912 (faster eluting isomer) and Example 913 (slower eluting isomer) by chiral HPLC was 87% hexane/IPA (Chiralcel OD column).

### Preparation of Examples 914-916

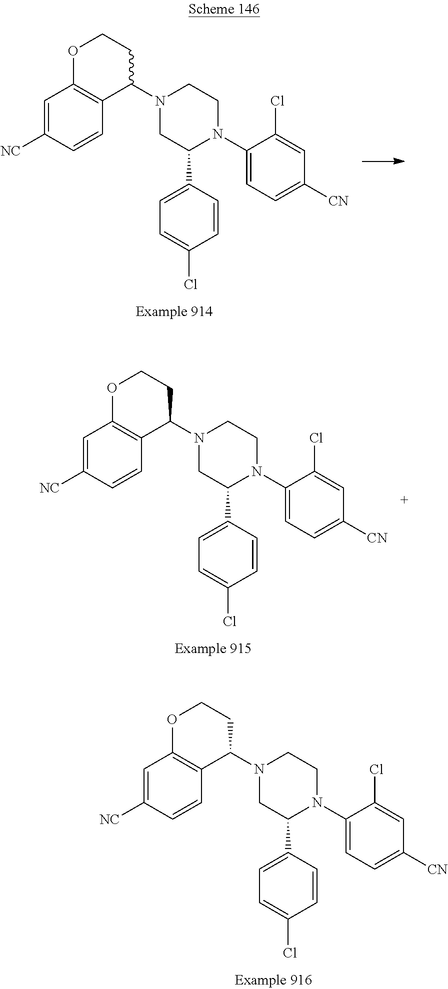 [SMILES: N#Cc1ccc(N2CCN(C3CCOc4cc(C#N)ccc43)C[C@H]2c2ccc(Cl)cc2)c(Cl)c1 ; N#Cc1ccc(N2CCN([C@@H]3CCOc4cc(C#N)ccc43)C[C@H]2c2ccc(Cl)cc2)c(Cl)c1 ; N#Cc1ccc(N2CCN([C@H]3CCOc4cc(C#N)ccc43)C[C@H]2c2ccc(Cl)cc2)c(Cl)c1]

Example 914 was prepared in the same manner as Example 397 in Scheme 68 except that 7-cyano-4-chromanone was used instead of 4-chromanone and Example 305 was used instead of Example 304. The 1:1 mixture of diastereomers (Examples 915 and 916) were separated by chiral preparative HPLC [Chiralcel OD, 85% hexane/IPA, 50 mL/min, 254 nm] to provide a faster eluting and a slower eluting isomer.

### Preparation of Example 917-919

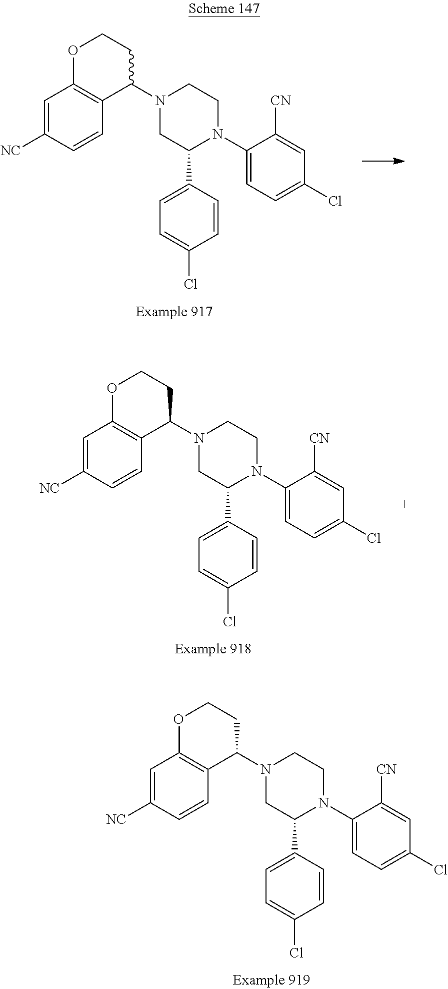 [SMILES: N#Cc1ccc2c(c1)OCCC2N1CCN(c2ccc(Cl)cc2C#N)[C@H](c2ccc(Cl)cc2)C1 ; N#Cc1ccc2c(c1)OCC[C@H]2N1CCN(c2ccc(Cl)cc2C#N)[C@H](c2ccc(Cl)cc2)C1 ; N#Cc1ccc2c(c1)OCC[C@@H]2N1CCN(c2ccc(Cl)cc2C#N)[C@H](c2ccc(Cl)cc2)C1]

Example 917 was prepared in the same manner as Example 397 in Scheme 68 except that 7-cyano-4-chromanone was used instead of 4-chromanone and Example 310 was used instead of Example 304. The 1:1 mixture of diastereomers (Examples 918 and 919) were separated by chiral preparative HPLC [Chiralcel OD, 85% hexane/IPA, 50 mL/min, 254 nm] to provide a faster eluting and a slower eluting isomer.

### Preparation of Example 920

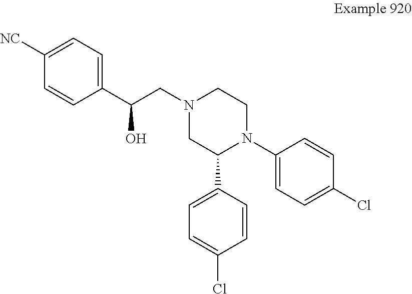 [SMILES: N#Cc1ccc([C@H](O)CN2CCN(c3ccc(Cl)cc3)[C@H](c3ccc(Cl)cc3)C2)cc1]

Example 920 was prepared in the same manner as Example 800 in scheme 88 except that Example 803 was used instead of Example 306. The eluent for silica gel chromatography was 30% EtOAc/hex and the eluent for the HPLC purification was 15% IPA/hexane [Chiralcel OD column].

### Preparation of Example 921

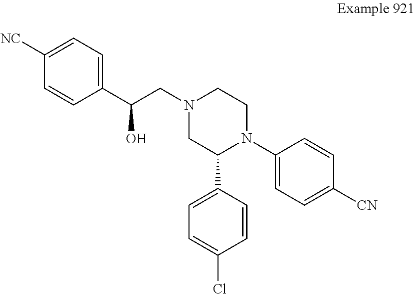 [SMILES: N#Cc1ccc([C@H](O)CN2CCN(c3ccc(C#N)cc3)[C@H](c3ccc(Cl)cc3)C2)cc1]

Example 921 was prepared in the same manner as Example 800 in scheme 88 except that Example 804 was used instead of Example 306. The eluent for silica gel chromatography was 65% EtOAc/hex and the eluent for the HPLC purification was 25% IPA/hexane [Chiralcel OD column].

### Preparation of Example 922

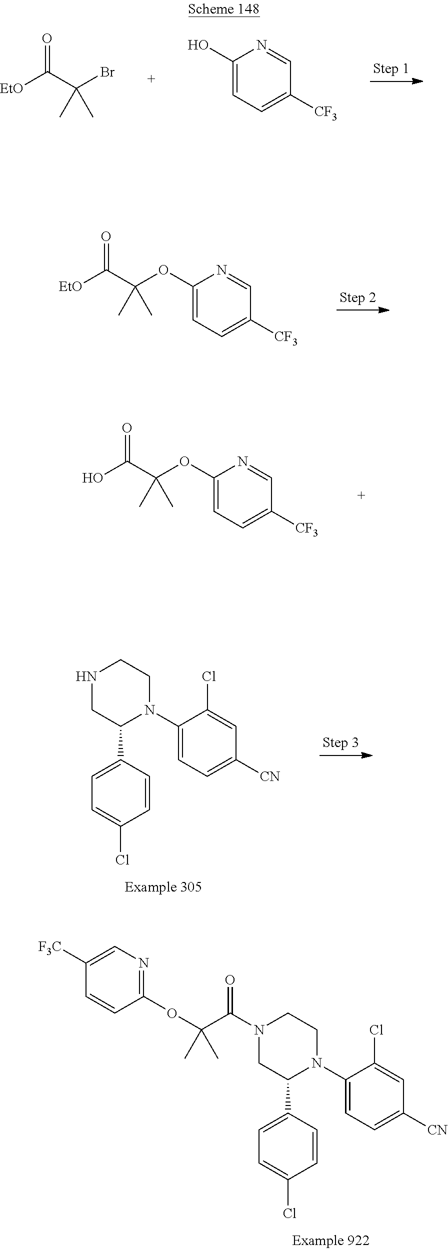

### Step 1

To 5-trifluoromethyl-2-hydroxypyridine (3.17 g, 19.4 mmol) in acetonitrile (24 mL) was added ethyl-2-bromoisobutyrate (3.78 g, 19.4 mmol) and cesium carbonate (12.6 g, 38.9 mmol). The mixture was warmed to 50 C and stirred for 42 h. The reaction was cooled to room temperature and then added to water. The mixture was extracted with EtOAc. The organic layer was washed with water and brine, dried (MgSO₄), filtered, and concentrated in vacuo. The residue was purified by silica gel chromatography (0-5% EtOAc/hex over 30 minutes) to provide pyridyl ester (1.4 g, 5.1 mmol).

### Step 2

To the ester (1.4 g, 5.1 mmol) in acetonitrile (5 mL) and water (5 mL) was added 2 N LiOH (5 mL, 10.1 mmol). The reaction was heated to 50 C and stirred for 24 h. The reaction was then heated to 80 C and stirred for an additional 24 h. Finally, the reaction was heated to 90 C and stirred for an additional 24 h. The reaction was then cooled to room temperature and 1N HCl was added to acidify the reaction. The mixture was extracted with EtOAc. The organic layer was washed with water and brine, dried (MgSO₄), filtered, and concentrated in vacuo. The residue was purified by silica gel chromatography (5% MeOH/DCM) to provide the acid (0.6 g).

### Step 3

To the acid prepared in step 2 (0.15 g, 0.6 mmol) in acetonitrile (1 mL) was added piperazine Example 305 (0.10 g, 0.3 mmol), TEA (0.125 mL, HOBT (0.081 g, 0.6 mmol), and EDCI (0.115 g, 0.6 mmol). The reaction mixture was heated to 80 C and stirred for 18 h. The reaction was cooled to room temperature and 1N NaOH was added. The mixture was extracted with EtOAc. The organic layer was washed with water and brine, dried (MgSO₄), filtered, and concentrated in vacuo. The residue was purified by silica gel chromatography (0-50% EtOAc/hex over 30 minutes) to provide Example 922 (0.13 g, 0.23 mmol).

### Preparation of Example 923

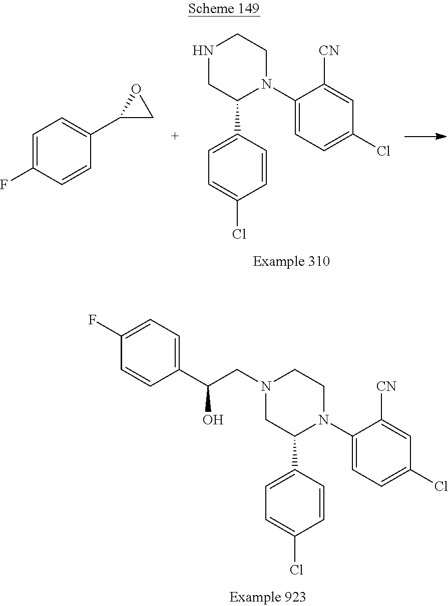 [SMILES: Fc1ccc([C@H]2CO2)cc1 ; N#Cc1cc(Cl)ccc1N1CCNC[C@H]1c1ccc(Cl)cc1 ; N#Cc1cc(Cl)ccc1N1CCN(C[C@@H](O)c2ccc(F)cc2)C[C@H]1c1ccc(Cl)cc1]

Example 923 was prepared in the same manner as Example 800 in scheme 88 except that piperazine Example 310 was used instead of Example 306 and the 4-fluorophenyl oxirane was used instead of the epoxide i. The fluorophenyl epoxide was prepared in the same manner as the cyanophenyl epoxide prepared in Scheme 79 except that 2-bromo-4′-fluoroacetophenone was used instead of 2-bromo-4′-cyanoacetophenone in step 1.

### Preparation of Example 923a

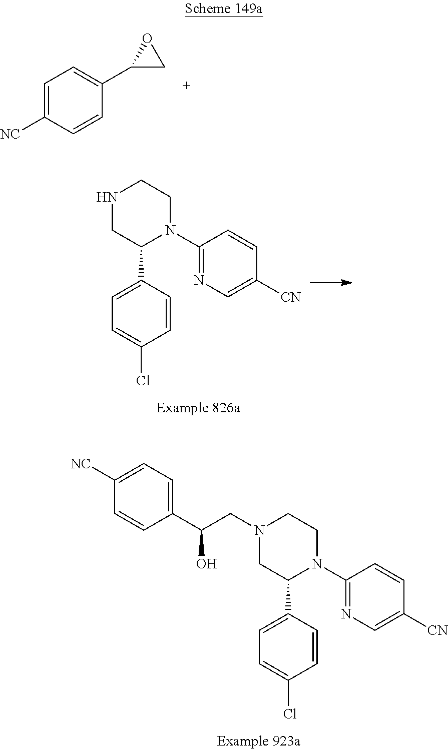 [SMILES: N#Cc1ccc([C@H]2CO2)cc1 ; N#Cc1ccc(N2CCNC[C@H]2c2ccc(Cl)cc2)nc1 ; N#Cc1ccc([C@H](O)CN2CCN(c3ccc(C#N)cn3)[C@H](c3ccc(Cl)cc3)C2)cc1]

Example 923a was prepared in the same manner as Example 776a in scheme 79 except that piperazine Example 826a was used instead of Example 304 in step 3.

### Preparation of 923b

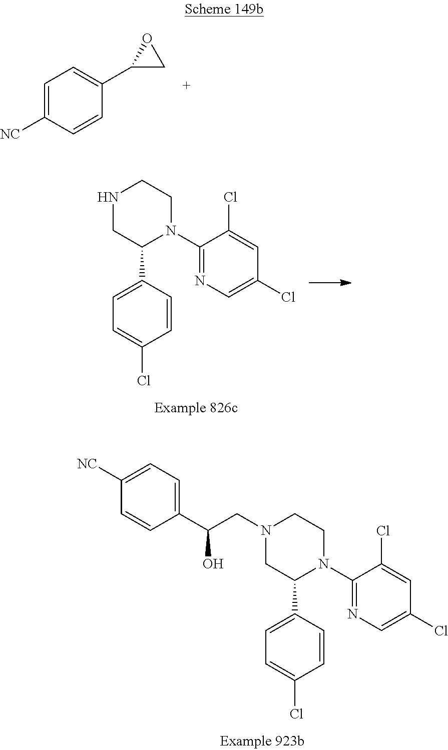 [SMILES: N#Cc1ccc([C@H]2CO2)cc1 ; Clc1ccc([C@@H]2CNCCN2c2ncc(Cl)cc2Cl)cc1 ; N#Cc1ccc([C@H](O)CN2CCN(c3ncc(Cl)cc3Cl)[C@H](c3ccc(Cl)cc3)C2)cc1]

Example 923b was prepared in the same manner as Example 776a in scheme 79 except that piperazine Example 826c was used instead of Example 304 in step 3.

### Preparation of 923c

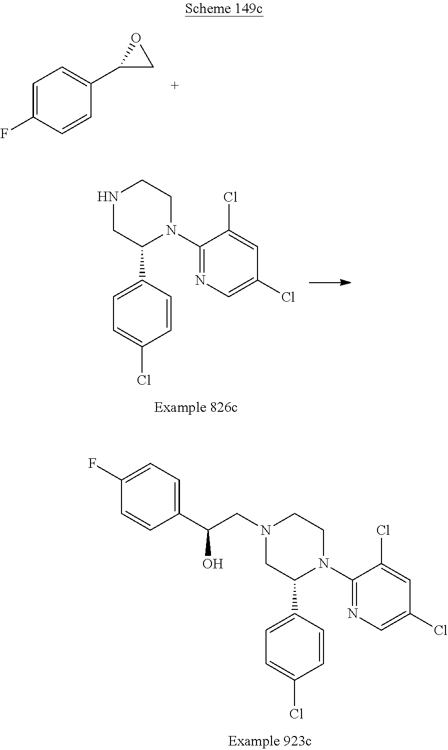 [SMILES: Fc1ccc([C@H]2CO2)cc1 ; Clc1ccc([C@@H]2CNCCN2c2ncc(Cl)cc2Cl)cc1 ; O[C@H](CN1CCN(c2ncc(Cl)cc2Cl)[C@H](c2ccc(Cl)cc2)C1)c1ccc(F)cc1]

Example 923c was prepared in the same manner as Example 800 in scheme 88 except that piperazine Example 826c was used instead of Example 306 and the 4-fluorophenyl oxirane was used instead of the epoxide i. The fluorophenyl epoxide was prepared in the same manner as the cyanophenyl epoxide prepared in Scheme 79 except that 2-bromo-4′-fluoroacetophenone was used instead of 2-bromo-4′-cyanoacetophenone in step 1.

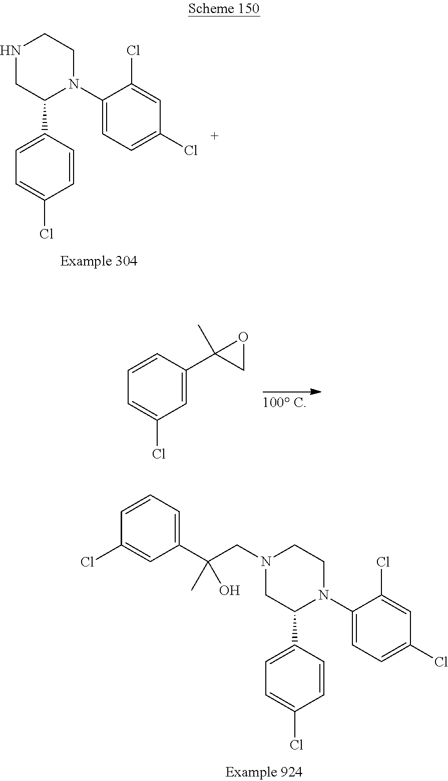

A sealed tube containing Example 304 (152 mg, 0.44 mmol) and the epoxide from Scheme 115 (75 mg, 0.44 mmol) was heated to 100° C. with stirring for 24 hours. The crude mixture was cooled to RT, dissolved in CH₂Cl₂ and transferred to a flash chromatography column. The crude product was purified via flash chromatography (SiO₂: gradient elution 100:0 to 80:20 hexanes:EtOAc) to afford Example 924 (156 mg).

The following examples were prepared in a similar manner to that described in

Scheme 150.

TABLE XXXV

| | Piperazine | | | |
|---|---|---|---|---|
| Ex. | Core | epoxide | Ex. | Structure |
| 304 | 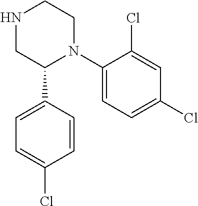 [SMILES: Clc1ccc([C@@H]2CNCCN2c2ccc(Cl)cc2Cl)cc1] | 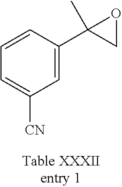 | 925 | 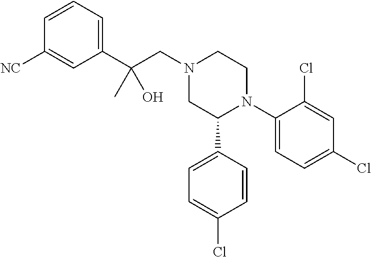 [SMILES: CC(O)(CN1CCN(c2ccc(Cl)cc2Cl)[C@H](c2ccc(Cl)cc2)C1)c1cccc(C#N)c1] |
| 304 | 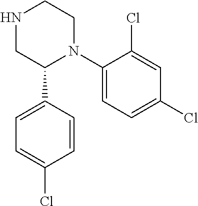 [SMILES: Clc1ccc([C@@H]2CNCCN2c2ccc(Cl)cc2Cl)cc1] | 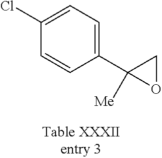 [SMILES: CC1(c2ccc(Cl)cc2)CO1] | 926 | 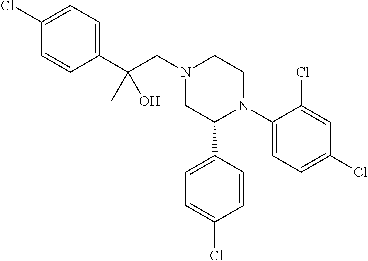 [SMILES: CC(O)(CN1CCN(c2ccc(Cl)cc2Cl)[C@H](c2ccc(Cl)cc2)C1)c1ccc(Cl)cc1] |
| 304 | 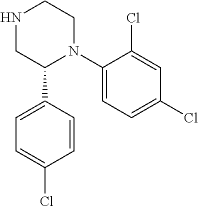 [SMILES: Clc1ccc([C@@H]2CNCCN2c2ccc(Cl)cc2Cl)cc1] | 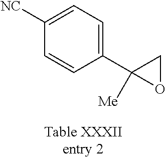 [SMILES: CC1(c2ccc(C#N)cc2)CO1] | 927 | 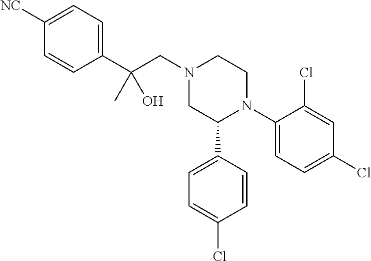 [SMILES: CC(O)(CN1CCN(c2ccc(Cl)cc2Cl)[C@H](c2ccc(Cl)cc2)C1)c1ccc(C#N)cc1] |
| 304 | 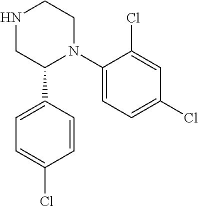 [SMILES: Clc1ccc([C@@H]2CNCCN2c2ccc(Cl)cc2Cl)cc1] | 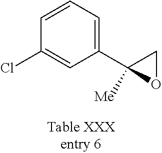 [SMILES: C[C@]1(c2cccc(Cl)c2)CO1] | 928 | 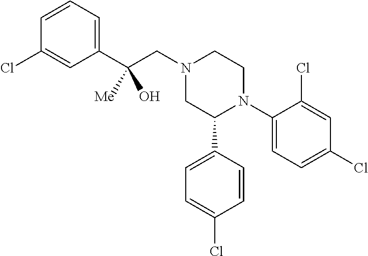 [SMILES: C[C@@](O)(CN1CCN(c2ccc(Cl)cc2Cl)[C@H](c2ccc(Cl)cc2)C1)c1cccc(Cl)c1] |
| 304 | 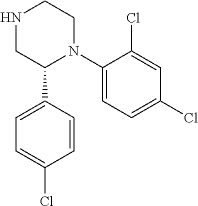 [SMILES: Clc1ccc([C@@H]2CNCCN2c2ccc(Cl)cc2Cl)cc1] | 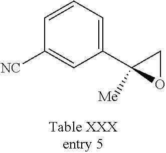 [SMILES: C[C@]1(c2cccc(C#N)c2)CO1] | 929 | 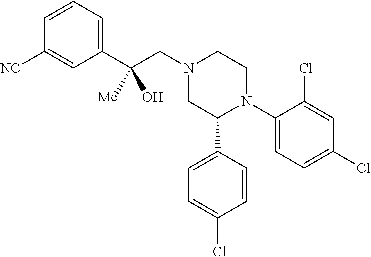 [SMILES: C[C@@](O)(CN1CCN(c2ccc(Cl)cc2Cl)[C@H](c2ccc(Cl)cc2)C1)c1cccc(C#N)c1] |
| 304 | 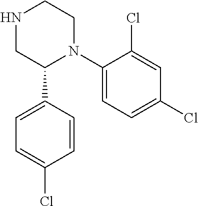 [SMILES: Clc1ccc([C@@H]2CNCCN2c2ccc(Cl)cc2Cl)cc1] | 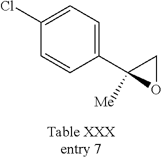 [SMILES: C[C@]1(c2ccc(Cl)cc2)CO1] | 930 | 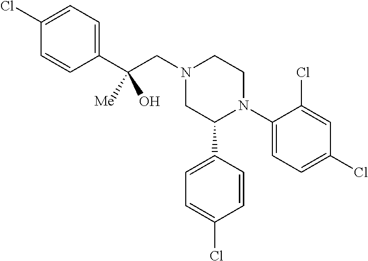 [SMILES: C[C@@](O)(CN1CCN(c2ccc(Cl)cc2Cl)[C@H](c2ccc(Cl)cc2)C1)c1ccc(Cl)cc1] |
| 304 | 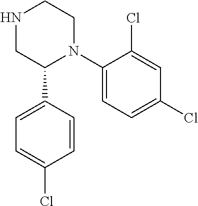 [SMILES: Clc1ccc([C@@H]2CNCCN2c2ccc(Cl)cc2Cl)cc1] | 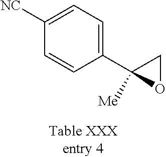 [SMILES: C[C@]1(c2ccc(C#N)cc2)CO1] | 931 | 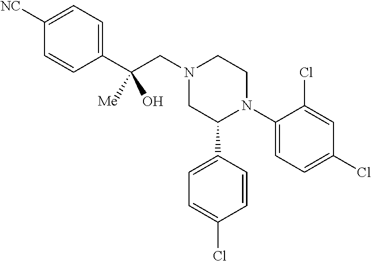 [SMILES: C[C@@](O)(CN1CCN(c2ccc(Cl)cc2Cl)[C@H](c2ccc(Cl)cc2)C1)c1ccc(C#N)cc1] |
| 304 | 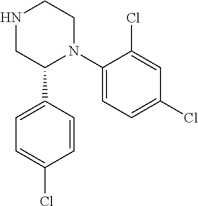 [SMILES: Clc1ccc([C@@H]2CNCCN2c2ccc(Cl)cc2Cl)cc1] | 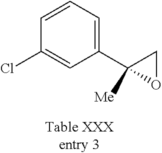 [SMILES: C[C@@]1(c2cccc(Cl)c2)CO1] | 932 | 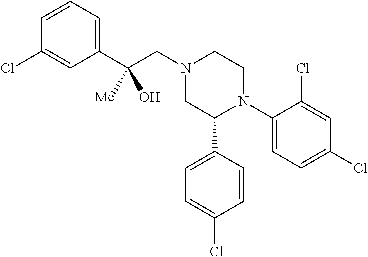 [SMILES: C[C@@](O)(CN1CCN(c2ccc(Cl)cc2Cl)[C@H](c2ccc(Cl)cc2)C1)c1cccc(Cl)c1] |
| 304 | 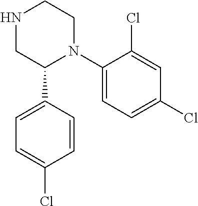 [SMILES: Clc1ccc([C@@H]2CNCCN2c2ccc(Cl)cc2Cl)cc1] | 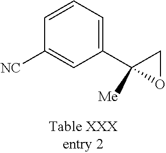 [SMILES: C[C@@]1(c2cccc(C#N)c2)CO1] | 933 | 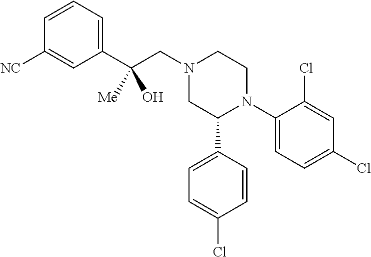 [SMILES: C[C@@](O)(CN1CCN(c2ccc(Cl)cc2Cl)[C@H](c2ccc(Cl)cc2)C1)c1cccc(C#N)c1] |
| 304 | 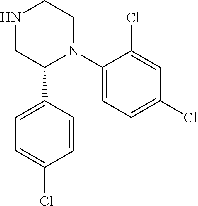 [SMILES: Clc1ccc([C@@H]2CNCCN2c2ccc(Cl)cc2Cl)cc1] | 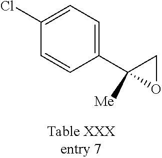 [SMILES: C[C@@]1(c2ccc(Cl)cc2)CO1] | 934 | 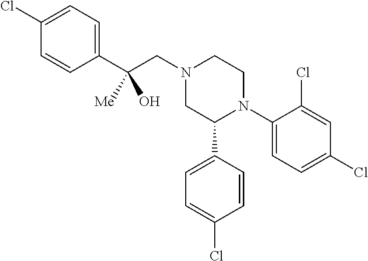 [SMILES: C[C@@](O)(CN1CCN(c2ccc(Cl)cc2Cl)[C@H](c2ccc(Cl)cc2)C1)c1ccc(Cl)cc1] |
| 304 | 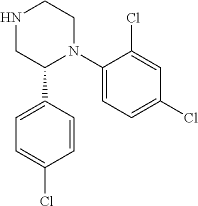 [SMILES: Clc1ccc([C@@H]2CNCCN2c2ccc(Cl)cc2Cl)cc1] | 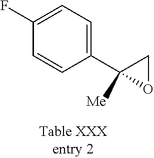 [SMILES: C[C@@]1(c2ccc(F)cc2)CO1] | 935 | 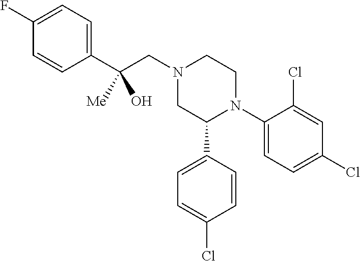 [SMILES: C[C@@](O)(CN1CCN(c2ccc(Cl)cc2Cl)[C@H](c2ccc(Cl)cc2)C1)c1ccc(F)cc1] |
| 305 | 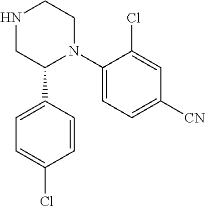 [SMILES: N#Cc1ccc(N2CCNC[C@H]2c2ccc(Cl)cc2)c(Cl)c1] | 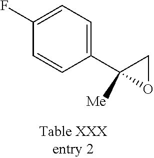 [SMILES: C[C@@]1(c2ccc(F)cc2)CO1] | 936 | 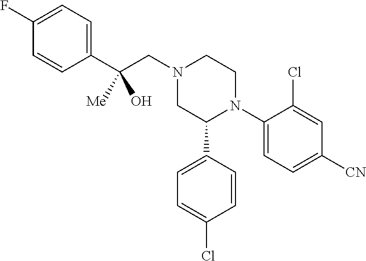 [SMILES: C[C@@](O)(CN1CCN(c2ccc(C#N)cc2Cl)[C@H](c2ccc(Cl)cc2)C1)c1ccc(F)cc1] |
| 305 | 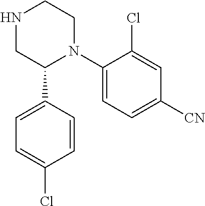 [SMILES: N#Cc1ccc(N2CCNC[C@H]2c2ccc(Cl)cc2)c(Cl)c1] | 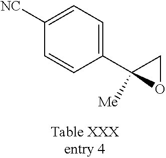 [SMILES: C[C@]1(c2ccc(C#N)cc2)CO1] | 937 | 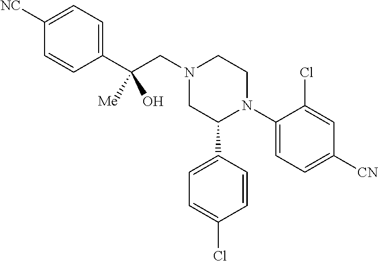 [SMILES: C[C@@](O)(CN1CCN(c2ccc(C#N)cc2Cl)[C@H](c2ccc(Cl)cc2)C1)c1ccc(C#N)cc1] |
| 305 | 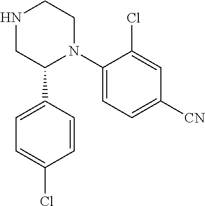 [SMILES: N#Cc1ccc(N2CCNC[C@H]2c2ccc(Cl)cc2)c(Cl)c1] | 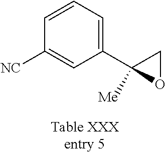 [SMILES: C[C@]1(c2cccc(C#N)c2)CO1] | 938 | 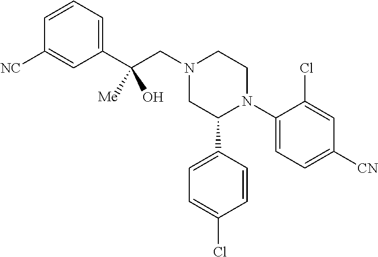 [SMILES: C[C@@](O)(CN1CCN(c2ccc(C#N)cc2Cl)[C@H](c2ccc(Cl)cc2)C1)c1cccc(C#N)c1] |
| 305 | 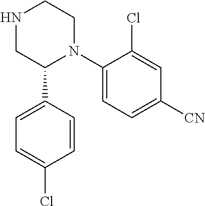 [SMILES: N#Cc1ccc(N2CCNC[C@H]2c2ccc(Cl)cc2)c(Cl)c1] | 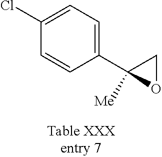 [SMILES: C[C@]1(c2ccc(Cl)cc2)CO1] | 939 | 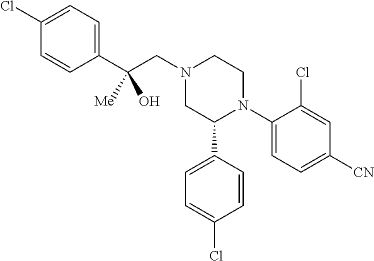 [SMILES: C[C@@](O)(CN1CCN(c2ccc(C#N)cc2Cl)[C@H](c2ccc(Cl)cc2)C1)c1ccc(Cl)cc1] |
| 304 | 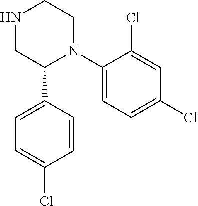 [SMILES: Clc1ccc([C@@H]2CNCCN2c2ccc(Cl)cc2Cl)cc1] | 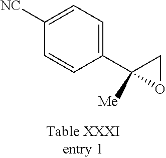 [SMILES: C[C@@]1(c2ccc(C#N)cc2)CO1] | 940 | 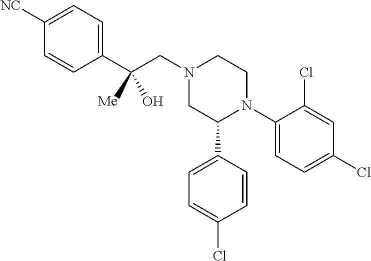 [SMILES: C[C@](O)(CN1CCN(c2ccc(Cl)cc2Cl)[C@H](c2ccc(Cl)cc2)C1)c1ccc(C#N)cc1] |
| 305 | 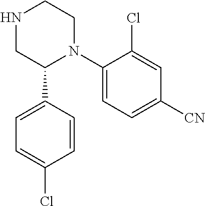 [SMILES: N#Cc1ccc(N2CCNC[C@H]2c2ccc(Cl)cc2)c(Cl)c1] | 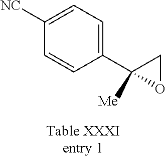 [SMILES: C[C@@]1(c2ccc(C#N)cc2)CO1] | 941 | 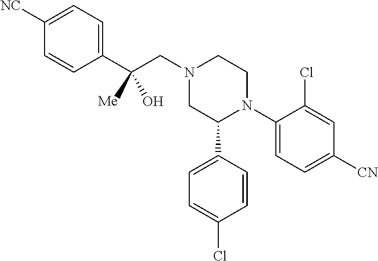 [SMILES: C[C@](O)(CN1CCN(c2ccc(C#N)cc2Cl)[C@H](c2ccc(Cl)cc2)C1)c1ccc(C#N)cc1] |
| 305 | 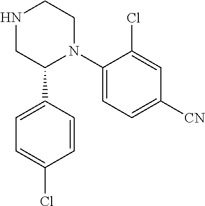 [SMILES: N#Cc1ccc(N2CCNC[C@H]2c2ccc(Cl)cc2)c(Cl)c1] | 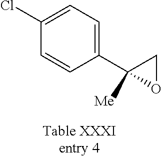 [SMILES: C[C@@]1(c2ccc(Cl)cc2)CO1] | 942 | 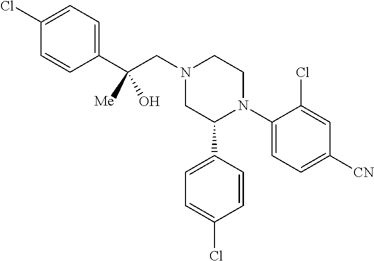 [SMILES: C[C@](O)(CN1CCN(c2ccc(C#N)cc2Cl)[C@H](c2ccc(Cl)cc2)C1)c1ccc(Cl)cc1] |
| 305 | 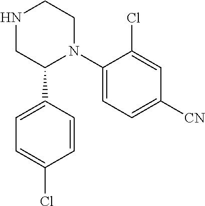 [SMILES: N#Cc1ccc(N2CCNC[C@H]2c2ccc(Cl)cc2)c(Cl)c1] | 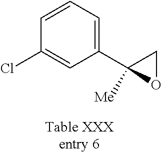 [SMILES: C[C@]1(c2cccc(Cl)c2)CO1] | 943 | 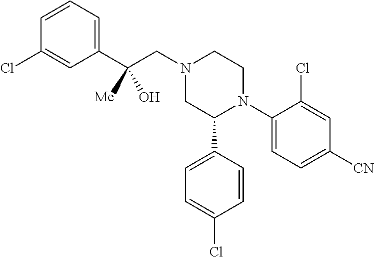 [SMILES: C[C@](O)(CN1CCN(c2ccc(C#N)cc2Cl)[C@H](c2ccc(Cl)cc2)C1)c1cccc(Cl)c1] |
| 304 | 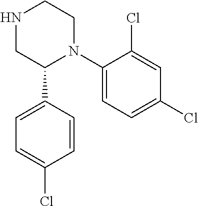 [SMILES: Clc1ccc([C@@H]2CNCCN2c2ccc(Cl)cc2Cl)cc1] | 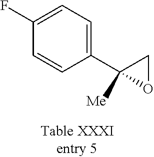 [SMILES: C[C@@]1(c2ccc(F)cc2)CO1] | 944 | 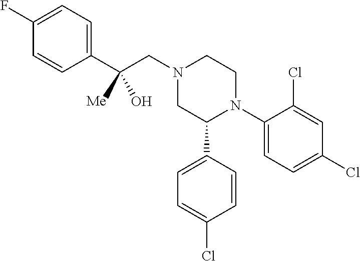 [SMILES: C[C@](O)(CN1CCN(c2ccc(Cl)cc2Cl)[C@H](c2ccc(Cl)cc2)C1)c1ccc(F)cc1] |
| 305 | 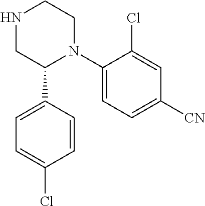 [SMILES: N#Cc1ccc(N2CCNC[C@H]2c2ccc(Cl)cc2)c(Cl)c1] | 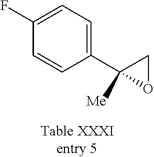 [SMILES: C[C@@]1(c2ccc(F)cc2)CO1] | 945 | 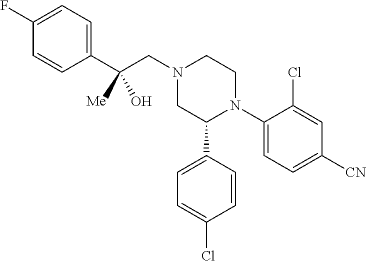 [SMILES: C[C@](O)(CN1CCN(c2ccc(C#N)cc2Cl)[C@H](c2ccc(Cl)cc2)C1)c1ccc(F)cc1] |
| 804 | 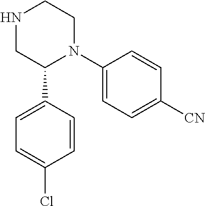 [SMILES: N#Cc1ccc(N2CCNC[C@H]2c2ccc(Cl)cc2)cc1] | 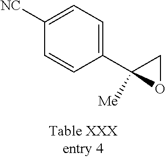 [SMILES: C[C@]1(c2ccc(C#N)cc2)CO1] | 946 | 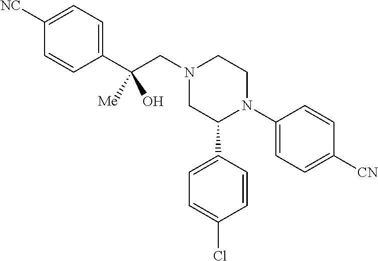 [SMILES: C[C@@](O)(CN1CCN(c2ccc(C#N)cc2)[C@H](c2ccc(Cl)cc2)C1)c1ccc(C#N)cc1] |
| 804 | 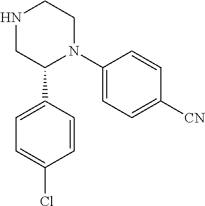 [SMILES: N#Cc1ccc(N2CCNC[C@H]2c2ccc(Cl)cc2)cc1] | 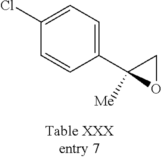 [SMILES: C[C@]1(c2ccc(Cl)cc2)CO1] | 947 | 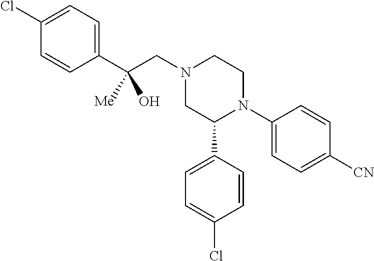 [SMILES: C[C@@](O)(CN1CCN(c2ccc(C#N)cc2)[C@H](c2ccc(Cl)cc2)C1)c1ccc(Cl)cc1] |
| 304 | 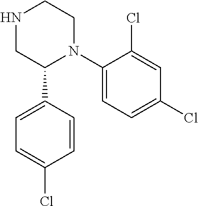 [SMILES: Clc1ccc([C@@H]2CNCCN2c2ccc(Cl)cc2Cl)cc1] | 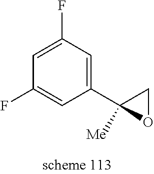 [SMILES: C[C@]1(c2cc(F)cc(F)c2)CO1] | 948 | 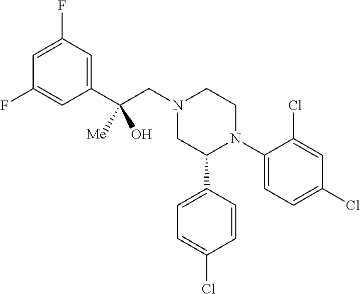 [SMILES: C[C@@](O)(CN1CCN(c2ccc(Cl)cc2Cl)[C@H](c2ccc(Cl)cc2)C1)c1cc(F)cc(F)c1] |
| 305 | 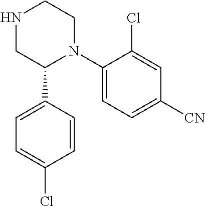 [SMILES: N#Cc1ccc(N2CCNC[C@H]2c2ccc(Cl)cc2)c(Cl)c1] | 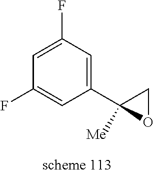 [SMILES: C[C@]1(c2cc(F)cc(F)c2)CO1] | 949 | 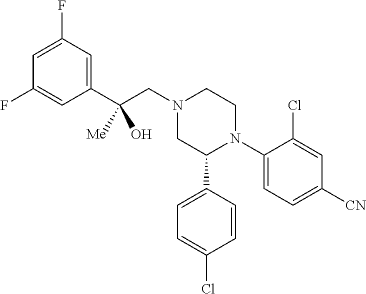 [SMILES: C[C@@](O)(CN1CCN(c2ccc(C#N)cc2Cl)[C@H](c2ccc(Cl)cc2)C1)c1cc(F)cc(F)c1] |
| 304 | 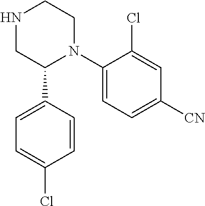 [SMILES: N#Cc1ccc(N2CCNC[C@H]2c2ccc(Cl)cc2)c(Cl)c1] | 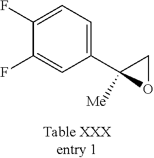 [SMILES: C[C@]1(c2ccc(F)c(F)c2)CO1] | 950 | 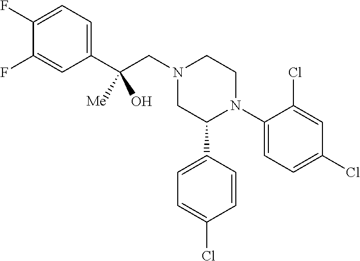 [SMILES: C[C@@](O)(CN1CCN(c2ccc(Cl)cc2Cl)[C@H](c2ccc(Cl)cc2)C1)c1ccc(F)c(F)c1] |
| 305 | 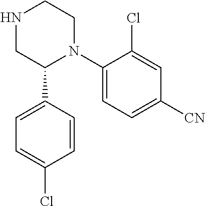 [SMILES: N#Cc1ccc(N2CCNC[C@H]2c2ccc(Cl)cc2)c(Cl)c1] | 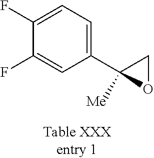 [SMILES: C[C@]1(c2ccc(F)c(F)c2)CO1] | 951 | 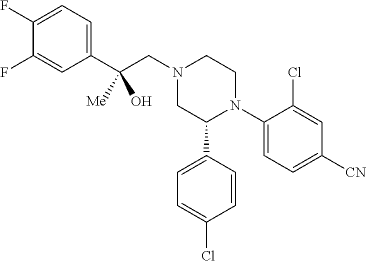 [SMILES: C[C@@](O)(CN1CCN(c2ccc(C#N)cc2Cl)[C@H](c2ccc(Cl)cc2)C1)c1ccc(F)c(F)c1] |
| 304 | 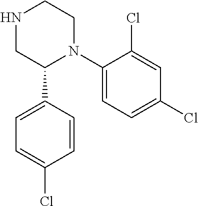 [SMILES: Clc1ccc([C@@H]2CNCCN2c2ccc(Cl)cc2Cl)cc1] | 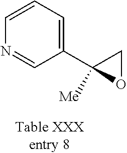 | 952 | 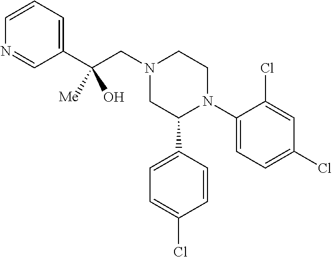 [SMILES: C[C@@](O)(CN1CCN(c2ccc(Cl)cc2Cl)[C@H](c2ccc(Cl)cc2)C1)c1cccnc1] |
| 305 | 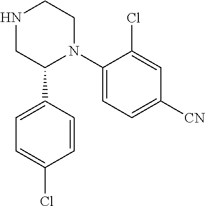 [SMILES: N#Cc1ccc(N2CCNC[C@H]2c2ccc(Cl)cc2)c(Cl)c1] | 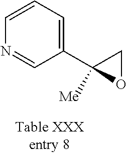 | 953 | 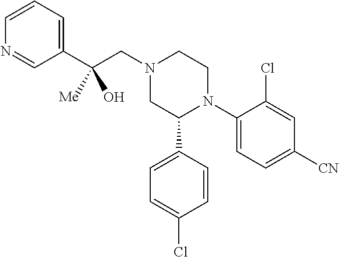 [SMILES: C[C@@](O)(CN1CCN(c2ccc(C#N)cc2Cl)[C@H](c2ccc(Cl)cc2)C1)c1cccnc1] |
| 803 | 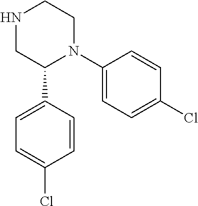 [SMILES: Clc1ccc([C@@H]2CNCCN2c2ccc(Cl)cc2)cc1] | 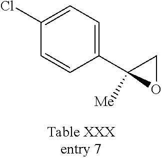 [SMILES: C[C@]1(c2ccc(Cl)cc2)CO1] | 954 | 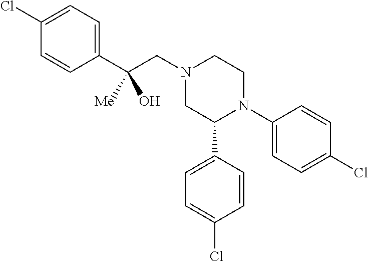 [SMILES: C[C@@](O)(CN1CCN(c2ccc(Cl)cc2)[C@H](c2ccc(Cl)cc2)C1)c1ccc(Cl)cc1] |
| 304 | 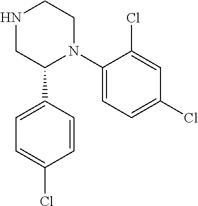 [SMILES: Clc1ccc([C@@H]2CNCCN2c2ccc(Cl)cc2Cl)cc1] | 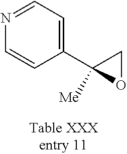 | 955 | 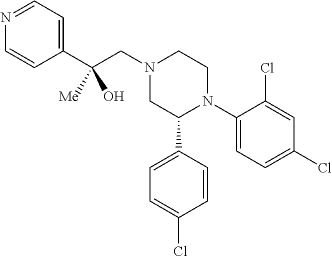 [SMILES: C[C@@](O)(CN1CCN(c2ccc(Cl)cc2Cl)[C@H](c2ccc(Cl)cc2)C1)c1ccncc1] |
| 305 | 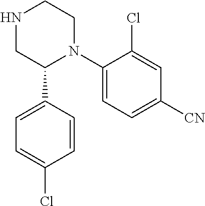 [SMILES: N#Cc1ccc(N2CCNC[C@H]2c2ccc(Cl)cc2)c(Cl)c1] | 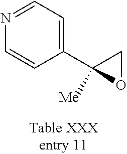 | 956 | 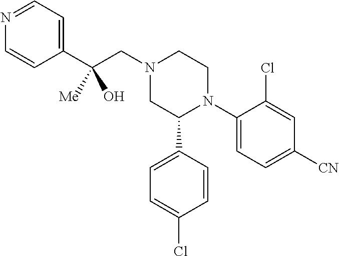 [SMILES: C[C@@](O)(CN1CCN(c2ccc(C#N)cc2Cl)[C@H](c2ccc(Cl)cc2)C1)c1ccncc1] |
| 304 | 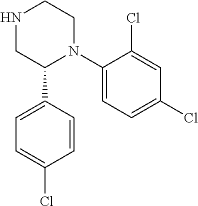 [SMILES: Clc1ccc([C@@H]2CNCCN2c2ccc(Cl)cc2Cl)cc1] | 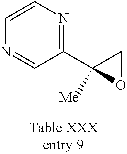 | 957 | 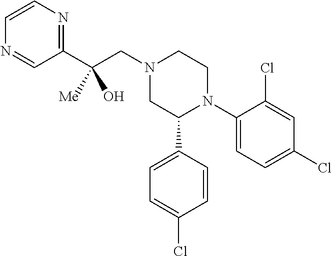 [SMILES: C[C@@](O)(CN1CCN(c2ccc(Cl)cc2Cl)[C@H](c2ccc(Cl)cc2)C1)c1cnccn1] |
| 305 | 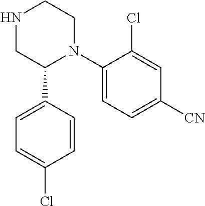 [SMILES: N#Cc1ccc(N2CCNC[C@H]2c2ccc(Cl)cc2)c(Cl)c1] | 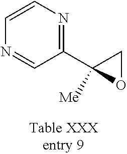 | 958 | 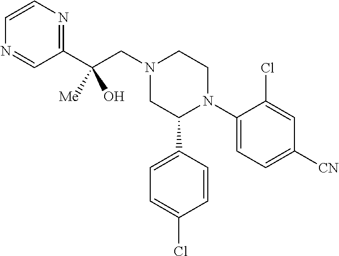 [SMILES: C[C@@](O)(CN1CCN(c2ccc(C#N)cc2Cl)[C@H](c2ccc(Cl)cc2)C1)c1cnccn1] |
| 803 | 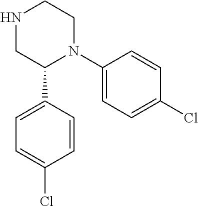 [SMILES: Clc1ccc([C@@H]2CNCCN2c2ccc(Cl)cc2)cc1] | 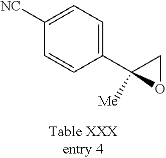 [SMILES: C[C@]1(c2ccc(C#N)cc2)CO1] | 959 | 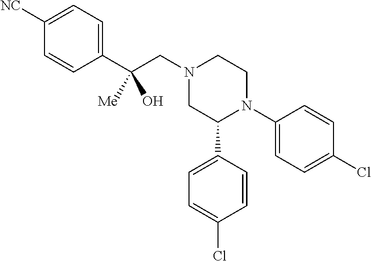 [SMILES: C[C@@](O)(CN1CCN(c2ccc(Cl)cc2)[C@H](c2ccc(Cl)cc2)C1)c1ccc(C#N)cc1] |
| 803 | 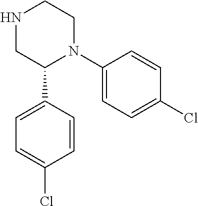 [SMILES: Clc1ccc([C@@H]2CNCCN2c2ccc(Cl)cc2)cc1] | 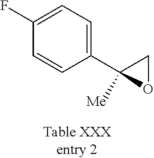 [SMILES: C[C@]1(c2ccc(F)cc2)CO1] | 960 | 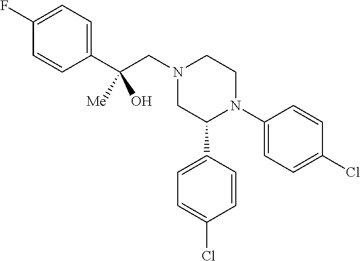 [SMILES: C[C@@](O)(CN1CCN(c2ccc(Cl)cc2)[C@H](c2ccc(Cl)cc2)C1)c1ccc(F)cc1] |
| 810 | 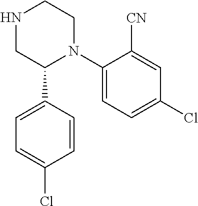 [SMILES: N#Cc1cc(Cl)ccc1N1CCNC[C@H]1c1ccc(Cl)cc1] | 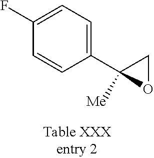 [SMILES: C[C@]1(c2ccc(F)cc2)CO1] | 961 | 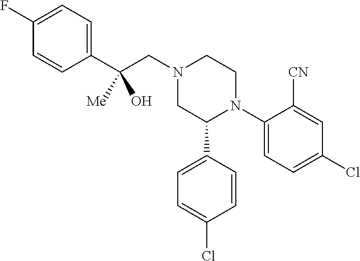 [SMILES: C[C@@](O)(CN1CCN(c2ccc(Cl)cc2C#N)[C@H](c2ccc(Cl)cc2)C1)c1ccc(F)cc1] |
| 810 | 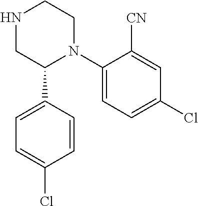 [SMILES: N#Cc1cc(Cl)ccc1N1CCNC[C@H]1c1ccc(Cl)cc1] | 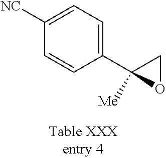 [SMILES: C[C@]1(c2ccc(C#N)cc2)CO1] | 962 | 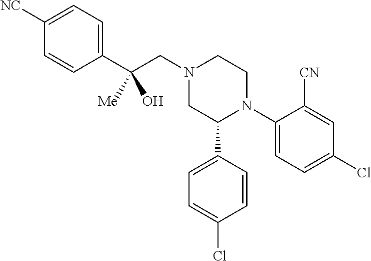 [SMILES: C[C@@](O)(CN1CCN(c2ccc(Cl)cc2C#N)[C@H](c2ccc(Cl)cc2)C1)c1ccc(C#N)cc1] |
| 305 | 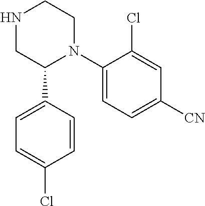 [SMILES: N#Cc1ccc(N2CCNC[C@H]2c2ccc(Cl)cc2)c(Cl)c1] | 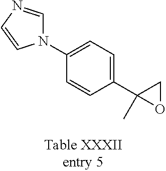 | 963 | 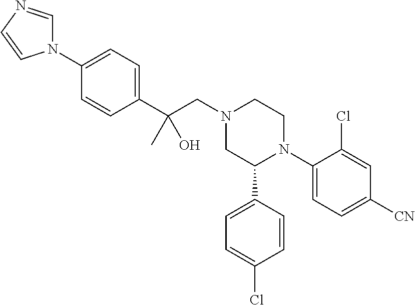 [SMILES: CC(O)(CN1CCN(c2ccc(C#N)cc2Cl)[C@H](c2ccc(Cl)cc2)C1)c1ccc(-n2ccnc2)cc1] |
| 304 | 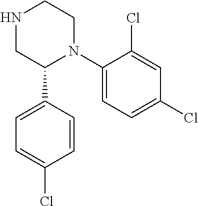 [SMILES: Clc1ccc([C@@H]2CNCCN2c2ccc(Cl)cc2Cl)cc1] | 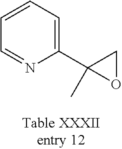 | 964 | 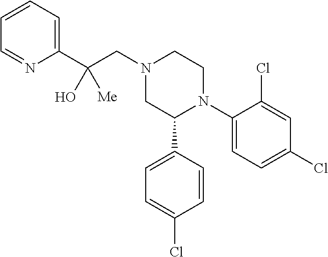 [SMILES: CC(O)(CN1CCN(c2ccc(Cl)cc2Cl)[C@H](c2ccc(Cl)cc2)C1)c1ccccn1] |
| 304 | 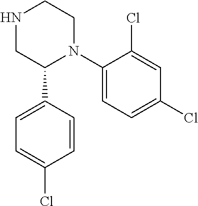 [SMILES: Clc1ccc([C@@H]2CNCCN2c2ccc(Cl)cc2Cl)cc1] | 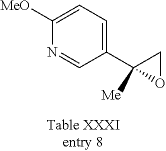 [SMILES: COc1ccc([C@]2(C)CO2)cn1] | 965 | 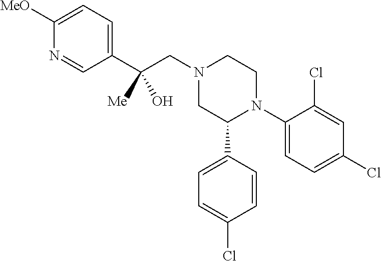 [SMILES: COc1ccc([C@@](C)(O)CN2CCN(c3ccc(Cl)cc3Cl)[C@H](c3ccc(Cl)cc3)C2)cn1] |
| 826b | 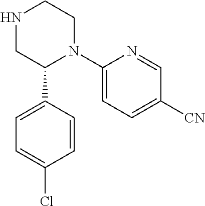 [SMILES: N#Cc1ccc(N2CCNC[C@H]2c2ccc(Cl)cc2)nc1] | 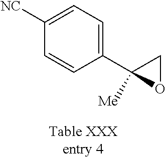 [SMILES: C[C@]1(c2ccc(C#N)cc2)CO1] | 965a | 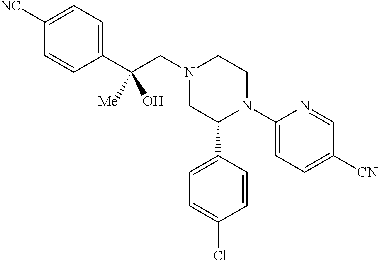 [SMILES: C[C@@](O)(CN1CCN(c2ccc(C#N)cn2)[C@H](c2ccc(Cl)cc2)C1)c1ccc(C#N)cc1] |
| 826a | 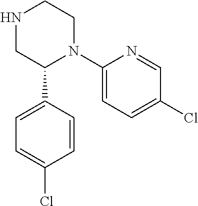 [SMILES: Clc1ccc([C@@H]2CNCCN2c2ccc(Cl)cn2)cc1] | 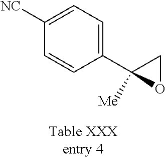 [SMILES: C[C@]1(c2ccc(C#N)cc2)CO1] | 965b | 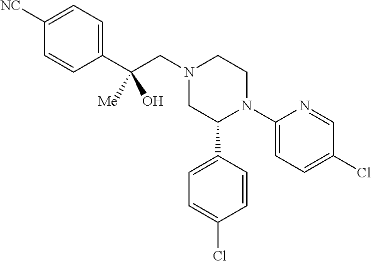 [SMILES: C[C@@](O)(CN1CCN(c2ccc(Cl)cn2)[C@H](c2ccc(Cl)cc2)C1)c1ccc(C#N)cc1] |
| 826a | 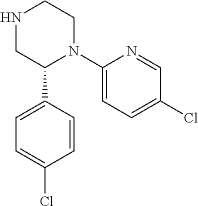 [SMILES: Clc1ccc([C@@H]2CNCCN2c2ccc(Cl)cn2)cc1] | 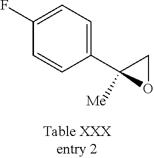 [SMILES: C[C@]1(c2ccc(F)cc2)CO1] | 965c | 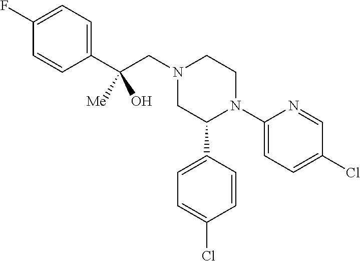 [SMILES: C[C@@](O)(CN1CCN(c2ccc(Cl)cn2)[C@H](c2ccc(Cl)cc2)C1)c1ccc(F)cc1] |
| 304 | 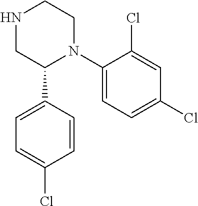 [SMILES: Clc1ccc([C@@H]2CNCCN2c2ccc(Cl)cc2Cl)cc1] | 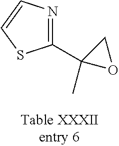 | 965d | 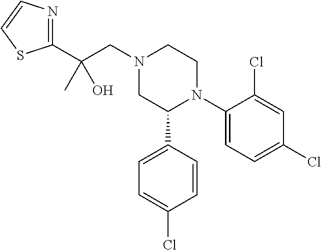 [SMILES: CC(O)(CN1CCN(c2ccc(Cl)cc2Cl)[C@H](c2ccc(Cl)cc2)C1)c1nccs1] |
| 305 | 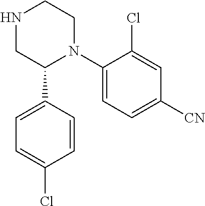 [SMILES: N#Cc1ccc(N2CCNC[C@H]2c2ccc(Cl)cc2)c(Cl)c1] | 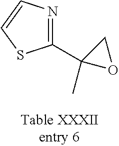 | 965e | 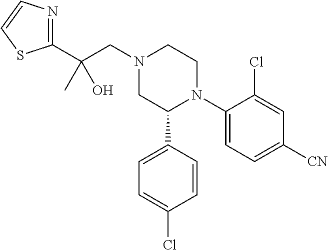 [SMILES: CC(O)(CN1CCN(c2ccc(C#N)cc2Cl)[C@H](c2ccc(Cl)cc2)C1)c1nccs1] |
| 305 | 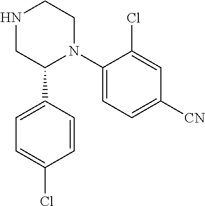 [SMILES: N#Cc1ccc(N2CCNC[C@H]2c2ccc(Cl)cc2)c(Cl)c1] | 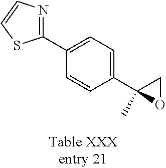 | 965f | 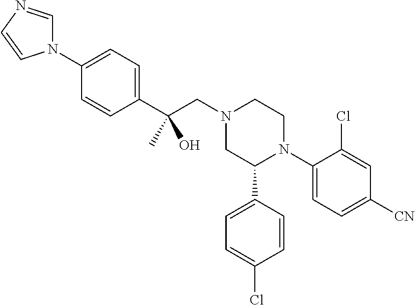 [SMILES: C[C@@](O)(CN1CCN(c2ccc(C#N)cc2Cl)[C@H](c2ccc(Cl)cc2)C1)c1ccc(-n2ccnc2)cc1] |

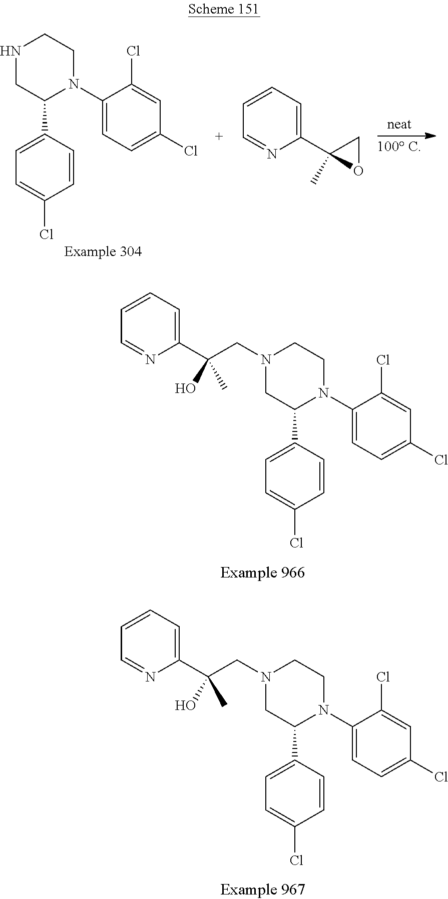

Example 966 and Example 967 were prepared from Example 304 and the epoxide from Table XXX entry 12 using a method similar to that described in Scheme 150 except Example 966 and Example 967 were separated via HPLC (Chiracel OD column, 95:5 hexanes:iPrOH).

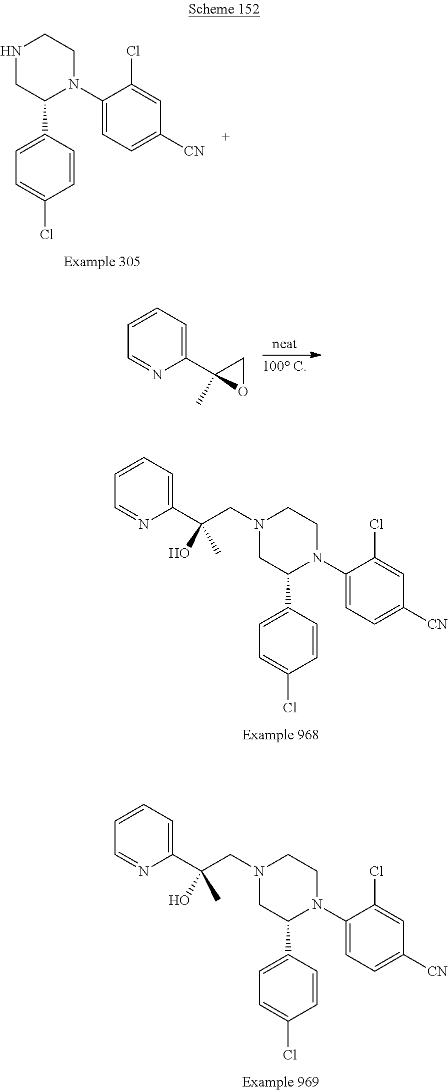

Example 968 and Example 969 were prepared from Example 305 and the epoxide from Table XXX entry 12 using a method similar to that described in Scheme 151 except Example 968 and Example 969 were separated via HPLC (Chiracel OD column, 90:10 hexanes:iPrOH).

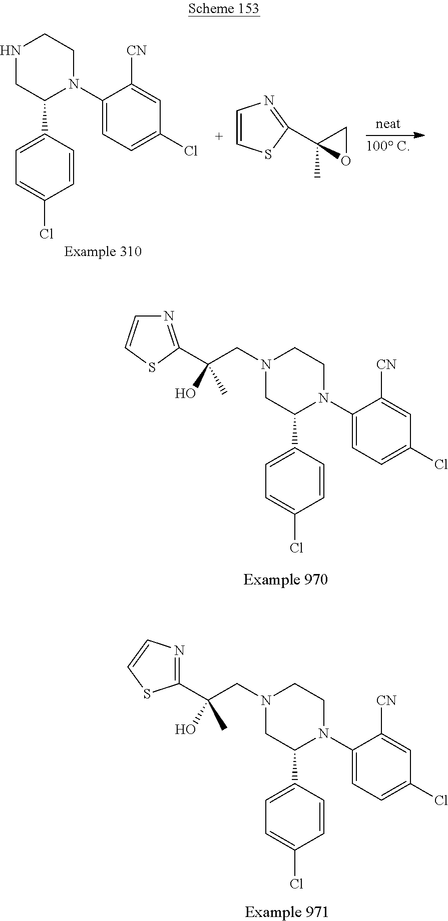

Example 970 and Example 971 were prepared from Example 310 and the epoxide from Table XXX entry 13 using a method similar to that described in Scheme 151 except Example 970 and Example 971 were separated via HPLC (Chiracel OD column, 90:10 hexanes:iPrOH).

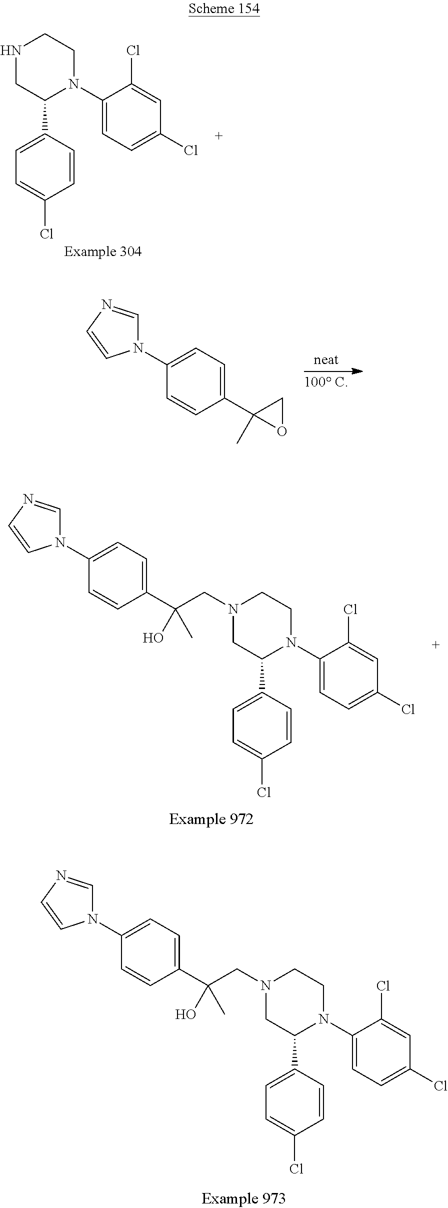 [SMILES: Clc1ccc([C@@H]2CNCCN2c2ccc(Cl)cc2Cl)cc1 ; CC1(c2ccc(-n3ccnc3)cc2)CO1 ; CC(O)(CN1CCN(c2ccc(Cl)cc2Cl)[C@H](c2ccc(Cl)cc2)C1)c1ccc(-n2ccnc2)cc1 ; CC(O)(CN1CCN(c2ccc(Cl)cc2Cl)[C@H](c2ccc(Cl)cc2)C1)c1ccc(-n2ccnc2)cc1]

Example 972 and Example 973 were prepared from Example 304 and the epoxide from Table XXX entry 5 using a method similar to that described in Scheme 151 except Example 972 and Example 973 were separated via HPLC (Chiracel OD column, 83:17 hexanes:iPrOH).

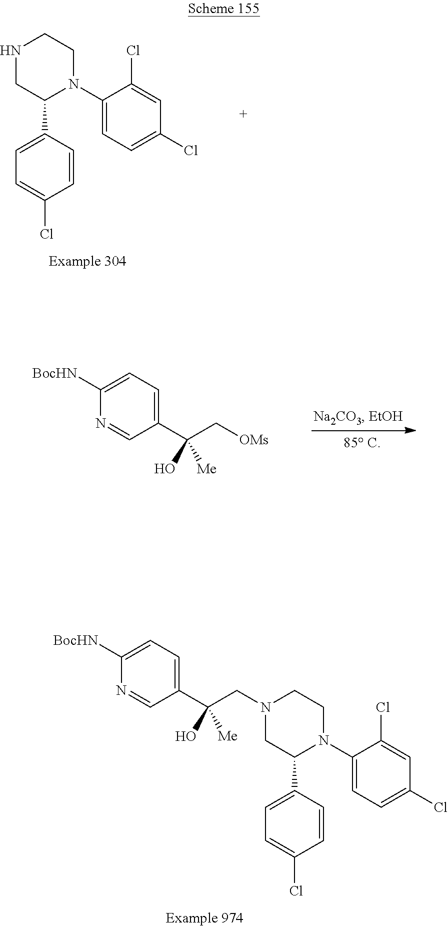 [SMILES: Clc1ccc([C@@H]2CNCCN2c2ccc(Cl)cc2Cl)cc1 ; CC(C)(C)OC(=O)Nc1ccc([C@](C)(O)COS(C)(=O)=O)cn1 ; CC(C)(C)OC(=O)Nc1ccc([C@](C)(O)CN2CCN(c3ccc(Cl)cc3Cl)[C@H](c3ccc(Cl)cc3)C2)cn1]

To a pressure tube containing a solution of Example 304 (108 mg, 0.32 mmol) in EtOH (3 mL) was added the mesylate from Scheme 109 (165 mg, 0.47 mmol) and Na₂CO₃ (50 mg, 0.47 mmol). The tube was sealed and the mixture was heated to 85° C. for 16 hours. The mixture was cooled to RT and concentrated. The residue was partitioned between water and EtOAc. The aqueous layer was extracted with EtOAc (3×). The combined organic layers were washed with brine, dried over Na₂SO₄, filtered and concentrated. The crude product was purified via flash chromatography (SiO₂: gradient elution 100:0 to 35:65 hexanes:EtOAc) to afford Example 974 (84 mg).

The following examples were prepared via a method similar to that described in Scheme 155.

TABLE XXXVI

| | Piperazine | | | |
|---|---|---|---|---|
| Ex. | Core | mesylate | Ex. | Structure |
| 305 | 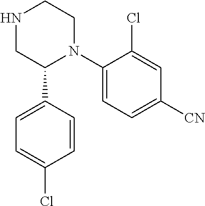 [SMILES: N#Cc1ccc(N2CCNC[C@H]2c2ccc(Cl)cc2)c(Cl)c1] | 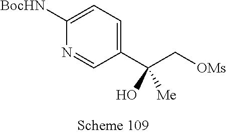 [SMILES: CC(C)(C)OC(=O)Nc1ccc([C@](C)(O)COS(C)(=O)=O)cn1] | 975 | 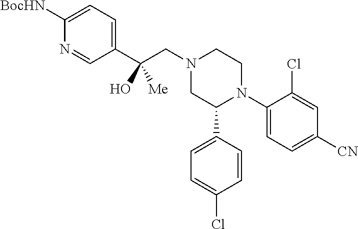 [SMILES: CC(C)(C)OC(=O)Nc1ccc([C@](C)(O)CN2CCN(c3ccc(C#N)cc3Cl)[C@H](c3ccc(Cl)cc3)C2)cn1] |
| 305 | 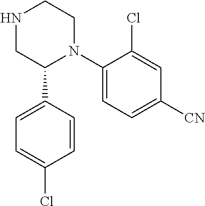 [SMILES: N#Cc1ccc(N2CCNC[C@H]2c2ccc(Cl)cc2)c(Cl)c1] | 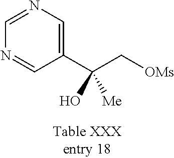 [SMILES: C[C@@](O)(COS(C)(=O)=O)c1cncnc1] | 976 | 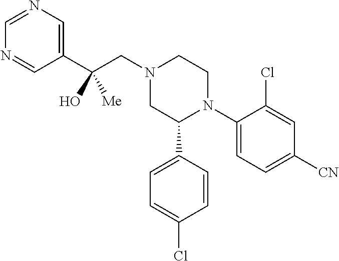 [SMILES: C[C@@](O)(CN1CCN(c2ccc(C#N)cc2Cl)[C@H](c2ccc(Cl)cc2)C1)c1cncnc1] |
| 305 | 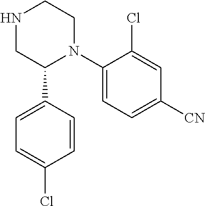 [SMILES: N#Cc1ccc(N2CCNC[C@H]2c2ccc(Cl)cc2)c(Cl)c1] | 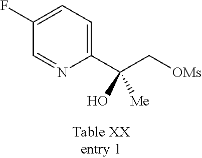 [SMILES: C[C@@](O)(COS(C)(=O)=O)c1ccc(F)cn1] | 977 | 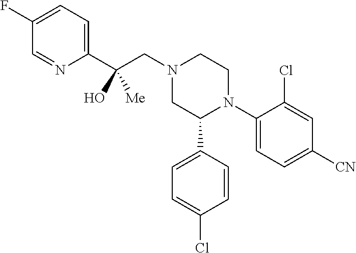 [SMILES: C[C@@](O)(CN1CCN(c2ccc(C#N)cc2Cl)[C@H](c2ccc(Cl)cc2)C1)c1ccc(F)cn1] |
| 304 | 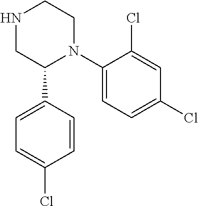 [SMILES: Clc1ccc([C@@H]2CNCCN2c2ccc(Cl)cc2Cl)cc1] | 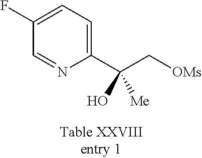 [SMILES: C[C@@](O)(COS(C)(=O)=O)c1ccc(F)cn1] | 978 | 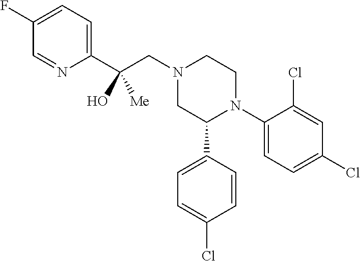 [SMILES: C[C@@](O)(CN1CCN(c2ccc(Cl)cc2Cl)[C@H](c2ccc(Cl)cc2)C1)c1ccc(F)cn1] |
| 305 | 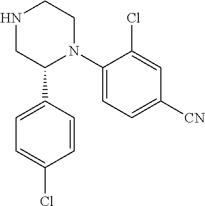 [SMILES: N#Cc1ccc(N2CCNC[C@H]2c2ccc(Cl)cc2)c(Cl)c1] | 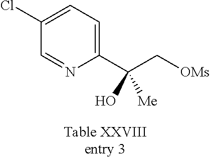 [SMILES: C[C@@](O)(COS(C)(=O)=O)c1ccc(Cl)cn1] | 979 | 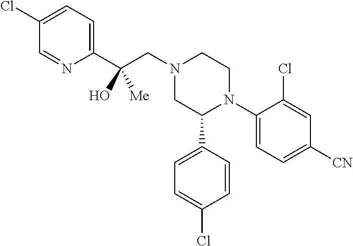 [SMILES: C[C@@](O)(CN1CCN(c2ccc(C#N)cc2Cl)[C@H](c2ccc(Cl)cc2)C1)c1ccc(Cl)cn1] |
| 304 | 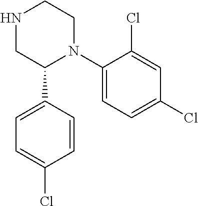 [SMILES: Clc1ccc([C@@H]2CNCCN2c2ccc(Cl)cc2Cl)cc1] | 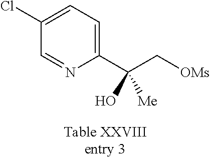 [SMILES: C[C@@](O)(COS(C)(=O)=O)c1ccc(Cl)cn1] | 980 | 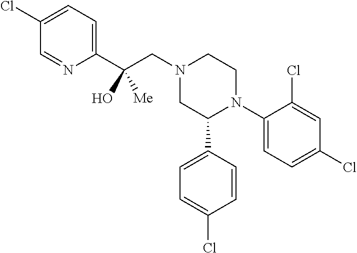 [SMILES: C[C@@](O)(CN1CCN(c2ccc(Cl)cc2Cl)[C@H](c2ccc(Cl)cc2)C1)c1ccc(Cl)cn1] |
| 305 | 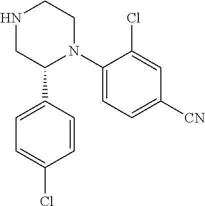 [SMILES: N#Cc1ccc(N2CCNC[C@H]2c2ccc(Cl)cc2)c(Cl)c1] | 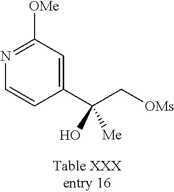 [SMILES: COc1cc([C@](C)(O)COS(C)(=O)=O)ccn1] | 981 | 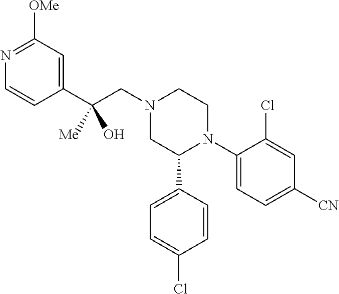 [SMILES: COc1cc([C@](C)(O)CN2CCN(c3ccc(C#N)cc3Cl)[C@H](c3ccc(Cl)cc3)C2)ccn1] |
| 305 | 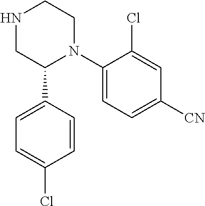 [SMILES: N#Cc1ccc(N2CCNC[C@H]2c2ccc(Cl)cc2)c(Cl)c1] | 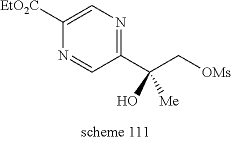 [SMILES: CCOC(=O)c1cnc([C@](C)(O)COS(C)(=O)=O)cn1] | 982 | 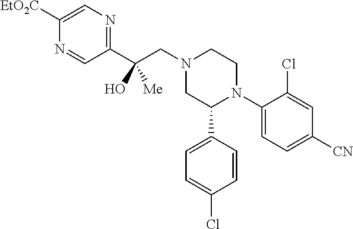 [SMILES: CCOC(=O)c1cnc([C@](C)(O)CN2CCN(c3ccc(C#N)cc3Cl)[C@H](c3ccc(Cl)cc3)C2)cn1] |
| 304 | 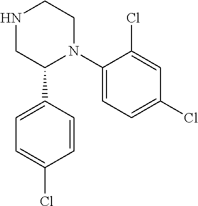 [SMILES: Clc1ccc([C@@H]2CNCCN2c2ccc(Cl)cc2Cl)cc1] | 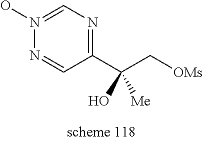 | 983 | 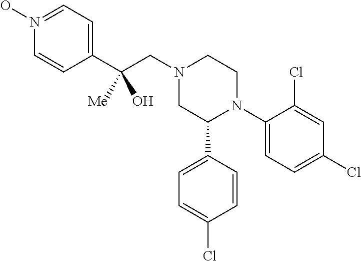 |
| 305 | 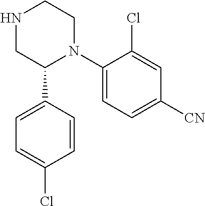 [SMILES: N#Cc1ccc(N2CCNC[C@H]2c2ccc(Cl)cc2)c(Cl)c1] | 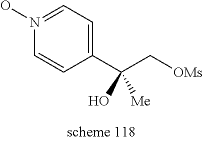 | 984 | 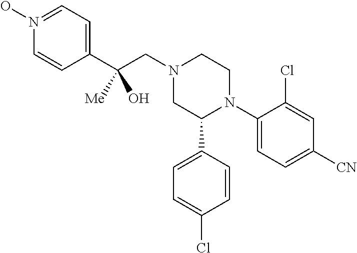 |
| 304 | 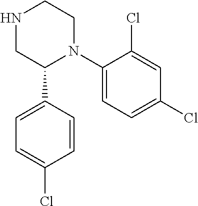 [SMILES: Clc1ccc([C@@H]2CNCCN2c2ccc(Cl)cc2Cl)cc1] | 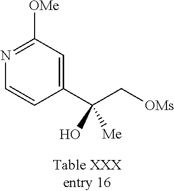 [SMILES: COc1cc([C@](C)(O)COS(C)(=O)=O)ccn1] | 985 | 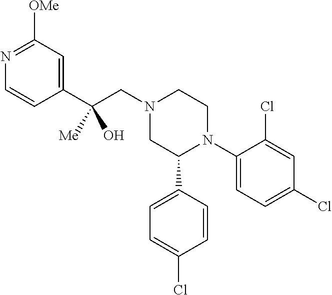 [SMILES: COc1cc([C@](C)(O)CN2CCN(c3ccc(Cl)cc3Cl)[C@H](c3ccc(Cl)cc3)C2)ccn1] |
| 304 | 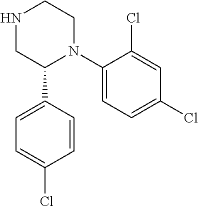 [SMILES: Clc1ccc([C@@H]2CNCCN2c2ccc(Cl)cc2Cl)cc1] | 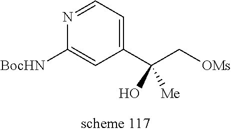 [SMILES: CC(C)(C)OC(=O)Nc1cc([C@](C)(O)COS(C)(=O)=O)ccn1] | 986 | 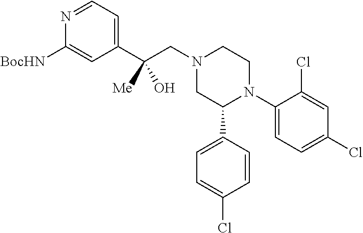 [SMILES: CC(C)(C)OC(=O)Nc1cc([C@@](C)(O)CN2CCN(c3ccc(Cl)cc3Cl)[C@H](c3ccc(Cl)cc3)C2)ccn1] |
| 305 | 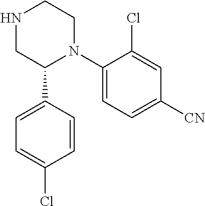 [SMILES: N#Cc1ccc(N2CCNC[C@H]2c2ccc(Cl)cc2)c(Cl)c1] | 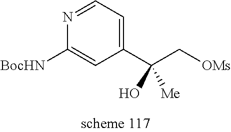 [SMILES: CC(C)(C)OC(=O)Nc1cc([C@](C)(O)COS(C)(=O)=O)ccn1] | 987 | 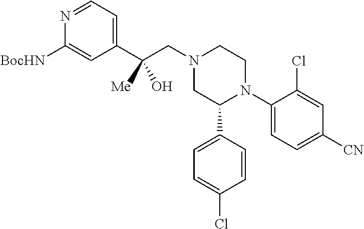 [SMILES: CC(C)(C)OC(=O)Nc1cc([C@@](C)(O)CN2CCN(c3ccc(C#N)cc3Cl)[C@H](c3ccc(Cl)cc3)C2)ccn1] |
| 304 | 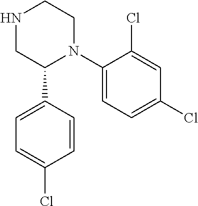 [SMILES: Clc1ccc([C@@H]2CNCCN2c2ccc(Cl)cc2Cl)cc1] | 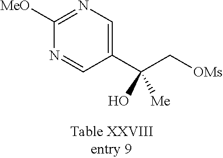 [SMILES: COc1ncc([C@](C)(O)COS(C)(=O)=O)cn1] | 988 | 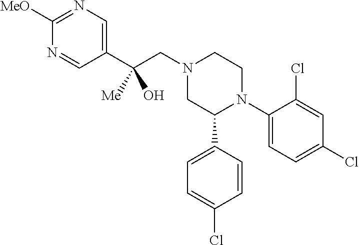 [SMILES: COc1ncc([C@](C)(O)CN2CCN(c3ccc(Cl)cc3Cl)[C@H](c3ccc(Cl)cc3)C2)cn1] |
| 305 | 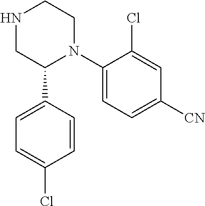 [SMILES: N#Cc1ccc(N2CCNC[C@H]2c2ccc(Cl)cc2)c(Cl)c1] | 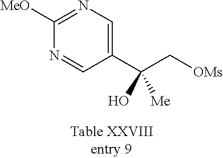 [SMILES: COc1ncc([C@](C)(O)COS(C)(=O)=O)cn1] | 989 | 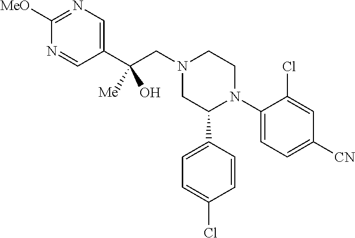 [SMILES: COc1ncc([C@](C)(O)CN2CCN(c3ccc(C#N)cc3Cl)[C@H](c3ccc(Cl)cc3)C2)cn1] |
| 310 | 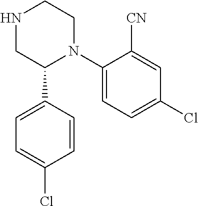 [SMILES: N#Cc1cc(Cl)ccc1N1CCNC[C@H]1c1ccc(Cl)cc1] | 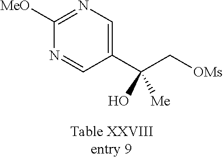 [SMILES: COc1ncc([C@](C)(O)COS(C)(=O)=O)cn1] | 990 | 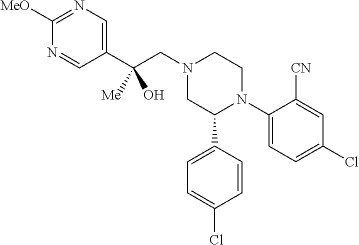 [SMILES: COc1ncc([C@](C)(O)CN2CCN(c3ccc(Cl)cc3C#N)[C@H](c3ccc(Cl)cc3)C2)cn1] |
| 310 | 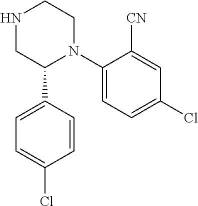 [SMILES: N#Cc1cc(Cl)ccc1N1CCNC[C@H]1c1ccc(Cl)cc1] | 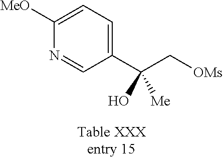 [SMILES: COc1ccc([C@](C)(O)COS(C)(=O)=O)cn1] | 991 | 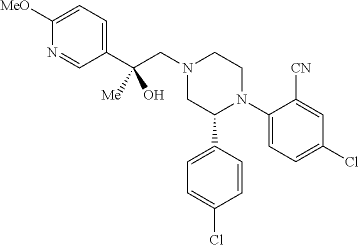 [SMILES: COc1ccc([C@](C)(O)CN2CCN(c3ccc(Cl)cc3C#N)[C@H](c3ccc(Cl)cc3)C2)cn1] |
| 310 | 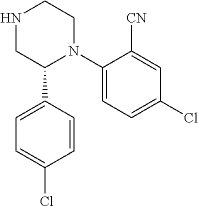 [SMILES: N#Cc1cc(Cl)ccc1N1CCNC[C@H]1c1ccc(Cl)cc1] | 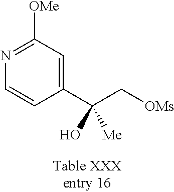 [SMILES: COc1cc([C@](C)(O)COS(C)(=O)=O)ccn1] | 992 | 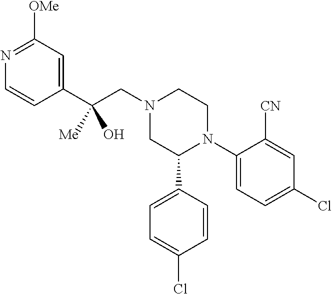 [SMILES: COc1cc([C@](C)(O)CN2CCN(c3ccc(Cl)cc3C#N)[C@H](c3ccc(Cl)cc3)C2)ccn1] |
| 310 | 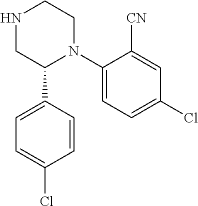 [SMILES: N#Cc1cc(Cl)ccc1N1CCNC[C@H]1c1ccc(Cl)cc1] | 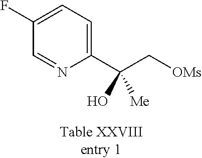 [SMILES: C[C@@](O)(COS(C)(=O)=O)c1ccc(F)cn1] | 993 | 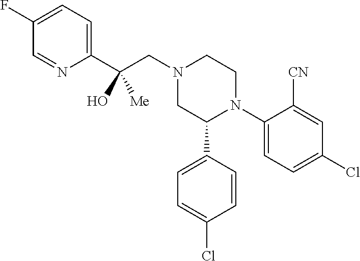 [SMILES: C[C@@](O)(CN1CCN(c2ccc(Cl)cc2C#N)[C@H](c2ccc(Cl)cc2)C1)c1ccc(F)cn1] |
| 310 | 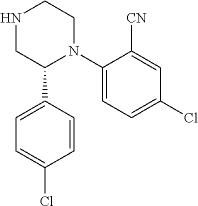 [SMILES: N#Cc1cc(Cl)ccc1N1CCNC[C@H]1c1ccc(Cl)cc1] | 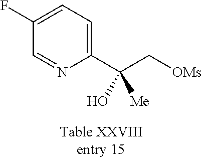 [SMILES: C[C@](O)(COS(C)(=O)=O)c1ccc(F)cn1] | 994 | 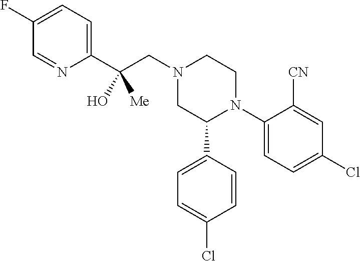 [SMILES: C[C@](O)(CN1CCN(c2ccc(Cl)cc2C#N)[C@H](c2ccc(Cl)cc2)C1)c1ccc(F)cn1] |
| 305 | 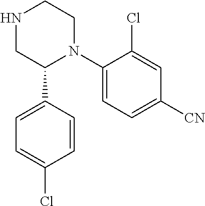 [SMILES: N#Cc1ccc(N2CCNC[C@H]2c2ccc(Cl)cc2)c(Cl)c1] | 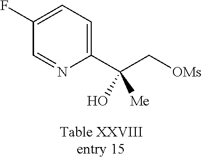 [SMILES: C[C@](O)(COS(C)(=O)=O)c1ccc(F)cn1] | 995 | 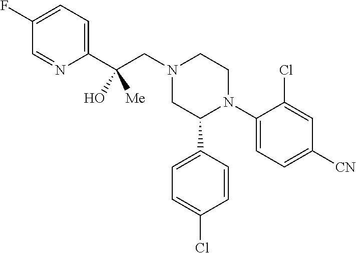 [SMILES: C[C@](O)(CN1CCN(c2ccc(C#N)cc2Cl)[C@H](c2ccc(Cl)cc2)C1)c1ccc(F)cn1] |
| 310 | 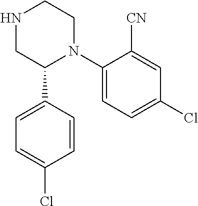 [SMILES: N#Cc1cc(Cl)ccc1N1CCNC[C@H]1c1ccc(Cl)cc1] | 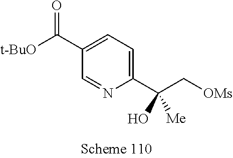 [SMILES: CC(C)(C)OC(=O)c1ccc([C@](C)(O)COS(C)(=O)=O)nc1] | 996 | 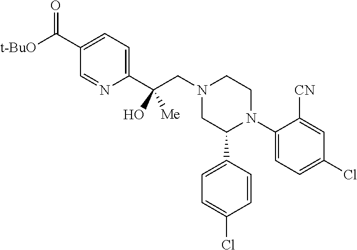 [SMILES: CC(C)(C)OC(=O)c1ccc([C@](C)(O)CN2CCN(c3ccc(Cl)cc3C#N)[C@H](c3ccc(Cl)cc3)C2)nc1] |
| 305 | 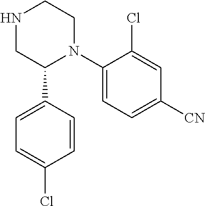 [SMILES: N#Cc1ccc(N2CCNC[C@H]2c2ccc(Cl)cc2)c(Cl)c1] | 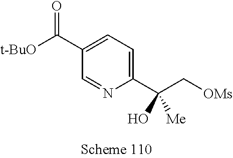 [SMILES: CC(C)(C)OC(=O)c1ccc([C@](C)(O)COS(C)(=O)=O)nc1] | 997 | 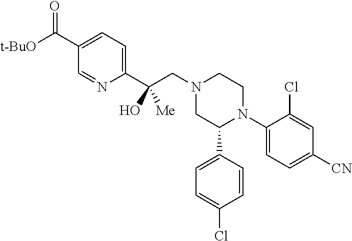 [SMILES: CC(C)(C)OC(=O)c1ccc([C@](C)(O)CN2CCN(c3ccc(C#N)cc3Cl)[C@H](c3ccc(Cl)cc3)C2)nc1] |
| 310 | 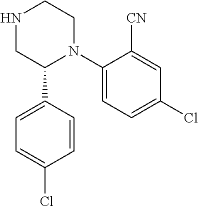 [SMILES: N#Cc1cc(Cl)ccc1N1CCNC[C@H]1c1ccc(Cl)cc1] | 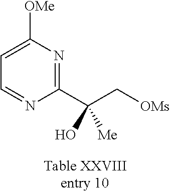 [SMILES: COc1ccnc([C@](C)(O)COS(C)(=O)=O)n1] | 998 | 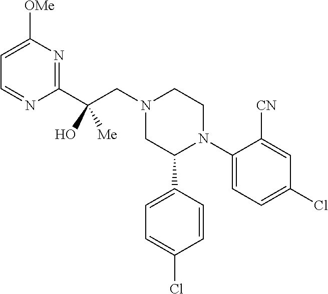 [SMILES: COc1ccnc([C@](C)(O)CN2CCN(c3ccc(Cl)cc3C#N)[C@H](c3ccc(Cl)cc3)C2)n1] |
| 305 | 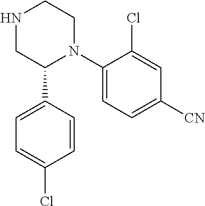 [SMILES: N#Cc1ccc(N2CCNC[C@H]2c2ccc(Cl)cc2)c(Cl)c1] | 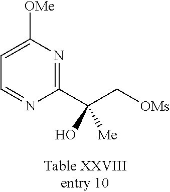 [SMILES: COc1ccnc([C@](C)(O)COS(C)(=O)=O)n1] | 999 | 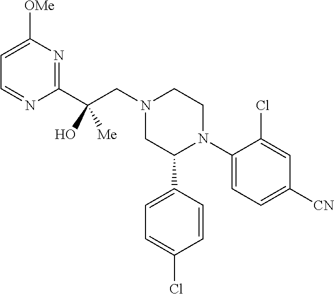 [SMILES: COc1ccnc([C@](C)(O)CN2CCN(c3ccc(C#N)cc3Cl)[C@H](c3ccc(Cl)cc3)C2)n1] |
| 304 | 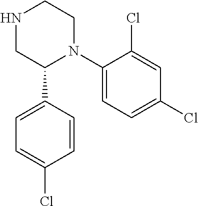 [SMILES: Clc1ccc([C@@H]2CNCCN2c2ccc(Cl)cc2Cl)cc1] | 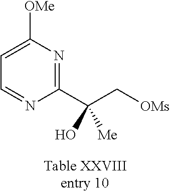 [SMILES: COc1ccnc([C@](C)(O)COS(C)(=O)=O)n1] | 1000 | 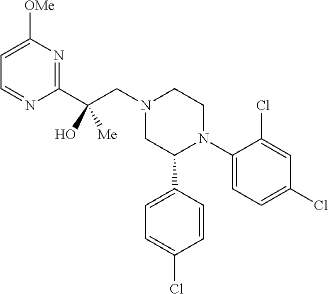 [SMILES: COc1ccnc([C@](C)(O)CN2CCN(c3ccc(Cl)cc3Cl)[C@H](c3ccc(Cl)cc3)C2)n1] |
| 803 | 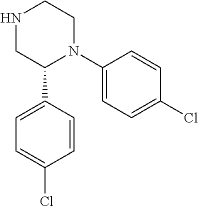 [SMILES: Clc1ccc([C@@H]2CNCCN2c2ccc(Cl)cc2)cc1] | 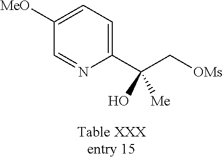 [SMILES: COc1ccc([C@](C)(O)COS(C)(=O)=O)nc1] | 1001 | 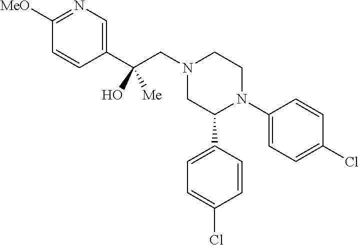 [SMILES: COc1ccc([C@](C)(O)CN2CCN(c3ccc(Cl)cc3)[C@H](c3ccc(Cl)cc3)C2)cn1] |
| 803 | 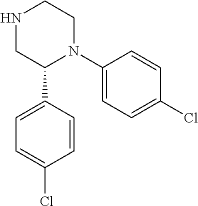 [SMILES: Clc1ccc([C@@H]2CNCCN2c2ccc(Cl)cc2)cc1] | 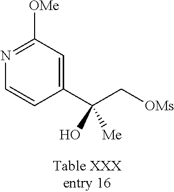 [SMILES: COc1cc([C@](C)(O)COS(C)(=O)=O)ccn1] | 1002 | 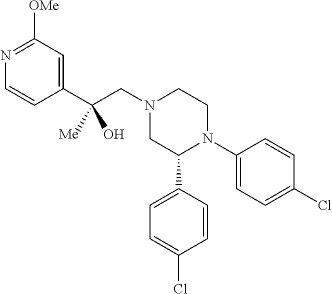 [SMILES: COc1cc([C@](C)(O)CN2CCN(c3ccc(Cl)cc3)[C@H](c3ccc(Cl)cc3)C2)ccn1] |
| 310 | 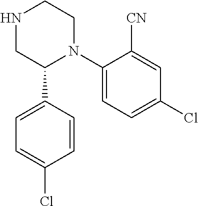 [SMILES: N#Cc1cc(Cl)ccc1N1CCNC[C@H]1c1ccc(Cl)cc1] | 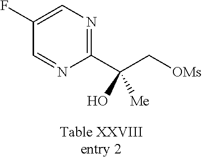 [SMILES: C[C@@](O)(COS(C)(=O)=O)c1ncc(F)cn1] | 1003 | 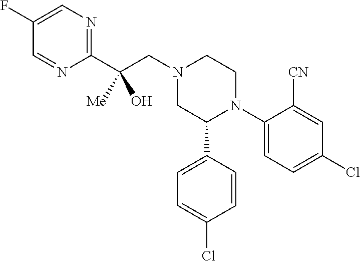 [SMILES: C[C@@](O)(CN1CCN(c2ccc(Cl)cc2C#N)[C@H](c2ccc(Cl)cc2)C1)c1ncc(F)cn1] |
| 305 | 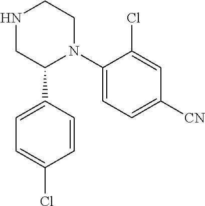 [SMILES: N#Cc1ccc(N2CCNC[C@H]2c2ccc(Cl)cc2)c(Cl)c1] | 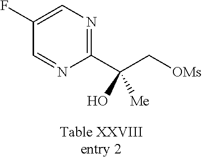 [SMILES: C[C@@](O)(COS(C)(=O)=O)c1ncc(F)cn1] | 1004 | 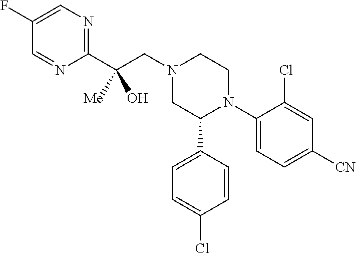 [SMILES: C[C@@](O)(CN1CCN(c2ccc(C#N)cc2Cl)[C@H](c2ccc(Cl)cc2)C1)c1ncc(F)cn1] |
| 304 | 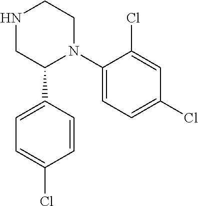 [SMILES: Clc1ccc([C@@H]2CNCCN2c2ccc(Cl)cc2Cl)cc1] | 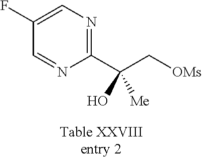 [SMILES: C[C@@](O)(COS(C)(=O)=O)c1ncc(F)cn1] | 1005 | 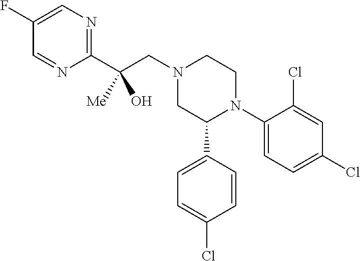 [SMILES: C[C@@](O)(CN1CCN(c2ccc(Cl)cc2Cl)[C@H](c2ccc(Cl)cc2)C1)c1ncc(F)cn1] |
| 310 | 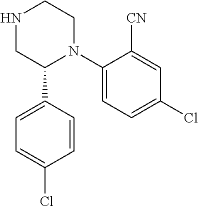 [SMILES: N#Cc1cc(Cl)ccc1N1CCNC[C@H]1c1ccc(Cl)cc1] | 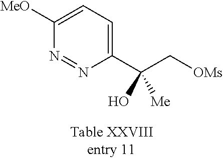 [SMILES: COc1ccc([C@](C)(O)COS(C)(=O)=O)nn1] | 1006 | 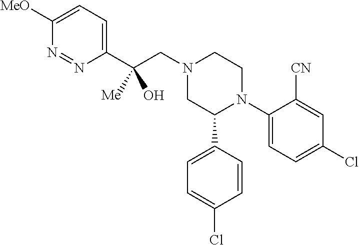 [SMILES: COc1ccc([C@](C)(O)CN2CCN(c3ccc(Cl)cc3C#N)[C@H](c3ccc(Cl)cc3)C2)nn1] |
| 305 | 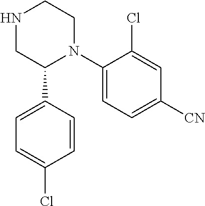 [SMILES: N#Cc1ccc(N2CCNC[C@H]2c2ccc(Cl)cc2)c(Cl)c1] | 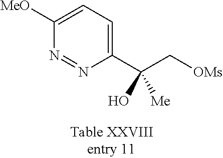 [SMILES: COc1ccc([C@](C)(O)COS(C)(=O)=O)nn1] | 1007 | 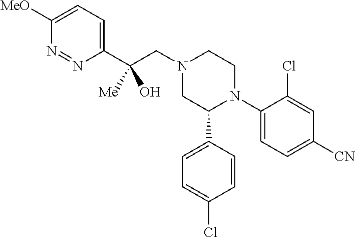 [SMILES: COc1ccc([C@](C)(O)CN2CCN(c3ccc(C#N)cc3Cl)[C@H](c3ccc(Cl)cc3)C2)nn1] |
| 304 | 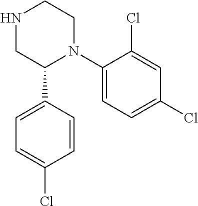 [SMILES: Clc1ccc([C@@H]2CNCCN2c2ccc(Cl)cc2Cl)cc1] | 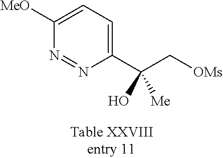 [SMILES: COc1ccc([C@](C)(O)COS(C)(=O)=O)nn1] | 1008 | 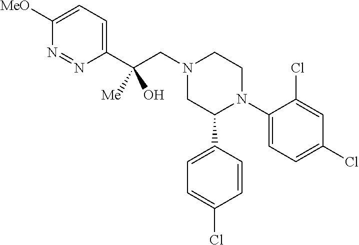 [SMILES: COc1ccc([C@](C)(O)CN2CCN(c3ccc(Cl)cc3Cl)[C@H](c3ccc(Cl)cc3)C2)nn1] |
| 310 | 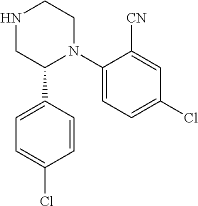 [SMILES: N#Cc1cc(Cl)ccc1N1CCNC[C@H]1c1ccc(Cl)cc1] | 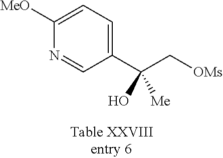 [SMILES: COc1ccc([C@](C)(O)COS(C)(=O)=O)cn1] | 1009 | 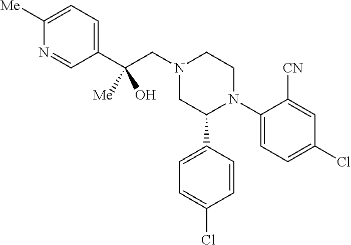 [SMILES: Cc1ccc([C@](C)(O)CN2CCN(c3ccc(Cl)cc3C#N)[C@H](c3ccc(Cl)cc3)C2)cn1] |
| 305 | 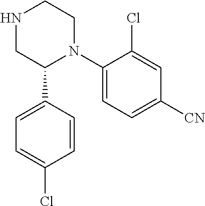 [SMILES: N#Cc1ccc(N2CCNC[C@H]2c2ccc(Cl)cc2)c(Cl)c1] | 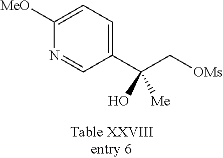 [SMILES: COc1ccc([C@](C)(O)COS(C)(=O)=O)cn1] | 1010 | 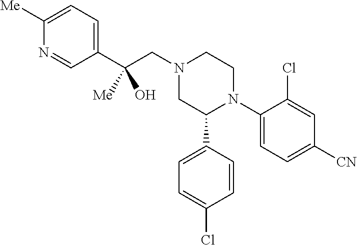 [SMILES: Cc1ccc([C@](C)(O)CN2CCN(c3ccc(C#N)cc3Cl)[C@H](c3ccc(Cl)cc3)C2)cn1] |
| 304 | 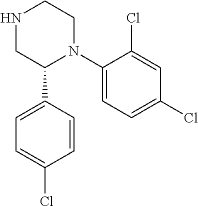 [SMILES: Clc1ccc([C@@H]2CNCCN2c2ccc(Cl)cc2Cl)cc1] | 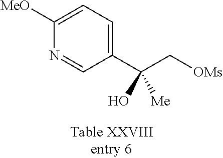 [SMILES: COc1ccc([C@](C)(O)COS(C)(=O)=O)cn1] | 1011 | 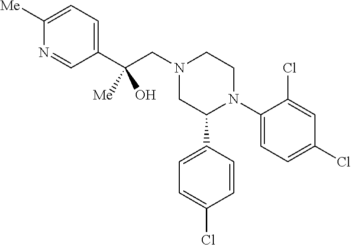 [SMILES: Cc1ccc([C@](C)(O)CN2CCN(c3ccc(Cl)cc3Cl)[C@H](c3ccc(Cl)cc3)C2)cn1] |
| 310 | 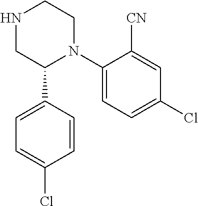 [SMILES: N#Cc1cc(Cl)ccc1N1CCNC[C@H]1c1ccc(Cl)cc1] | 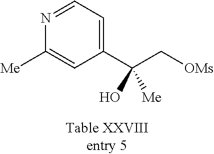 [SMILES: Cc1cc([C@](C)(O)COS(C)(=O)=O)ccn1] | 1012 | 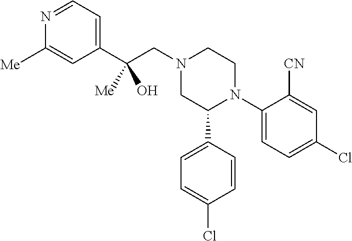 [SMILES: Cc1cc([C@](C)(O)CN2CCN(c3ccc(Cl)cc3C#N)[C@H](c3ccc(Cl)cc3)C2)ccn1] |
| 305 | 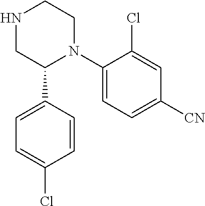 [SMILES: N#Cc1ccc(N2CCNC[C@H]2c2ccc(Cl)cc2)c(Cl)c1] | 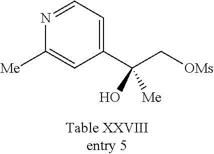 [SMILES: Cc1cc([C@](C)(O)COS(C)(=O)=O)ccn1] | 1013 | 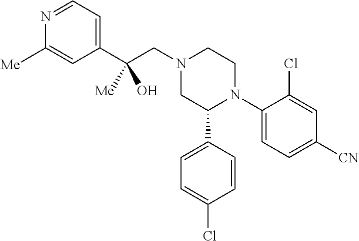 [SMILES: Cc1cc([C@](C)(O)CN2CCN(c3ccc(C#N)cc3Cl)[C@H](c3ccc(Cl)cc3)C2)ccn1] |
| 304 | 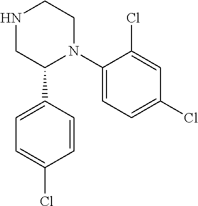 [SMILES: Clc1ccc([C@@H]2CNCCN2c2ccc(Cl)cc2Cl)cc1] | 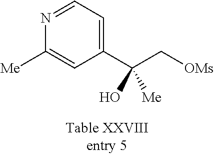 [SMILES: Cc1cc([C@](C)(O)COS(C)(=O)=O)ccn1] | 1014 | 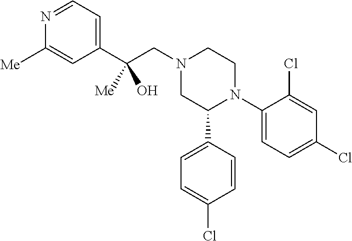 [SMILES: Cc1cc([C@](C)(O)CN2CCN(c3ccc(Cl)cc3Cl)[C@H](c3ccc(Cl)cc3)C2)ccn1] |
| 806 | 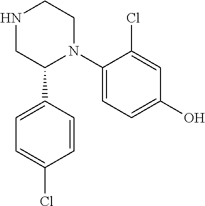 [SMILES: Oc1ccc(N2CCNC[C@H]2c2ccc(Cl)cc2)c(Cl)c1] | 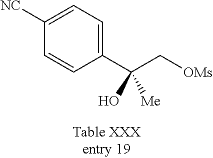 [SMILES: C[C@@](O)(COS(C)(=O)=O)c1ccc(C#N)cc1] | 1015 | 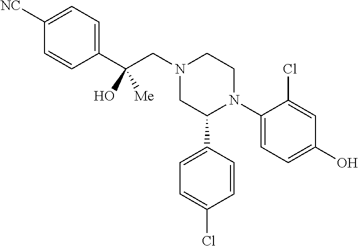 [SMILES: C[C@@](O)(CN1CCN(c2ccc(O)cc2Cl)[C@H](c2ccc(Cl)cc2)C1)c1ccc(C#N)cc1] |
| 806 | 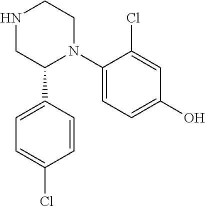 [SMILES: Oc1ccc(N2CCNC[C@H]2c2ccc(Cl)cc2)c(Cl)c1] | 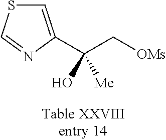 [SMILES: C[C@@](O)(COS(C)(=O)=O)c1cscn1] | 1016 | 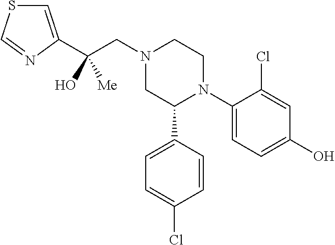 [SMILES: C[C@@](O)(CN1CCN(c2ccc(O)cc2Cl)[C@H](c2ccc(Cl)cc2)C1)c1cscn1] |
| 806 | 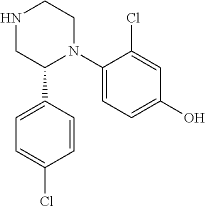 [SMILES: Oc1ccc(N2CCNC[C@H]2c2ccc(Cl)cc2)c(Cl)c1] | 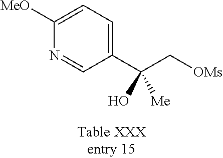 [SMILES: COc1ccc([C@](C)(O)COS(C)(=O)=O)cn1] | 1017 | 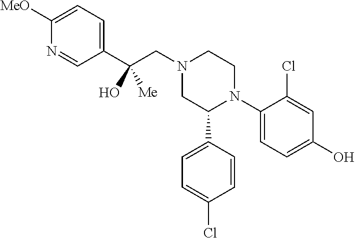 [SMILES: COc1ccc([C@](C)(O)CN2CCN(c3ccc(O)cc3Cl)[C@H](c3ccc(Cl)cc3)C2)cn1] |
| 813b | 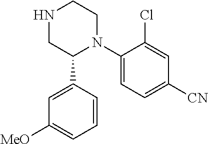 [SMILES: COc1cccc([C@@H]2CNCCN2c2ccc(C#N)cc2Cl)c1] | 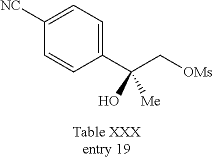 [SMILES: C[C@@](O)(COS(C)(=O)=O)c1ccc(C#N)cc1] | 1018 | 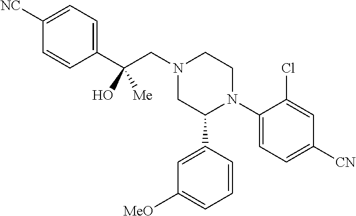 [SMILES: COc1cccc([C@@H]2CN(C[C@@](C)(O)c3ccc(C#N)cc3)CCN2c2ccc(C#N)cc2Cl)c1] |
| 813b | 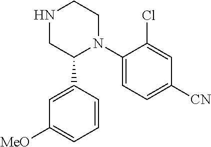 [SMILES: COc1cccc([C@@H]2CNCCN2c2ccc(C#N)cc2Cl)c1] | 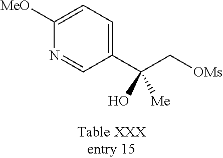 [SMILES: COc1ccc([C@](C)(O)COS(C)(=O)=O)cn1] | 1019 | 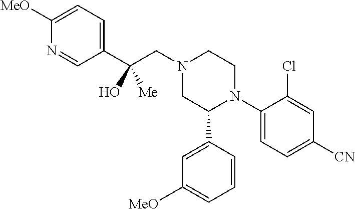 [SMILES: COc1cccc([C@@H]2CN(C[C@@](C)(O)c3ccc(OC)nc3)CCN2c2ccc(C#N)cc2Cl)c1] |
| 813b | 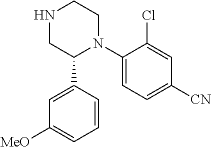 [SMILES: COc1cccc([C@@H]2CNCCN2c2ccc(C#N)cc2Cl)c1] | 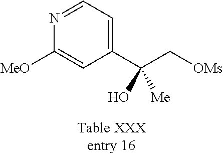 [SMILES: COc1cc([C@](C)(O)COS(C)(=O)=O)ccn1] | 1020 | 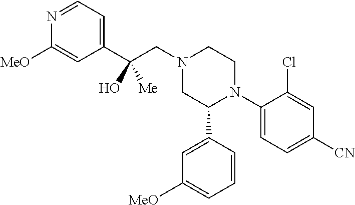 [SMILES: COc1cccc([C@@H]2CN(C[C@@](C)(O)c3ccnc(OC)c3)CCN2c2ccc(C#N)cc2Cl)c1] |
| 310 | 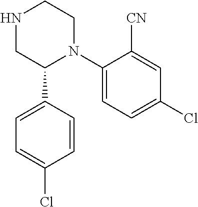 [SMILES: N#Cc1cc(Cl)ccc1N1CCNC[C@H]1c1ccc(Cl)cc1] | 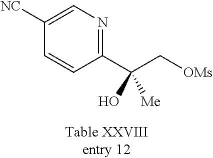 [SMILES: C[C@@](O)(COS(C)(=O)=O)c1ccc(C#N)cn1] | 1021 | 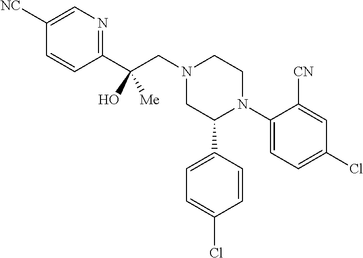 [SMILES: C[C@@](O)(CN1CCN(c2ccc(Cl)cc2C#N)[C@H](c2ccc(Cl)cc2)C1)c1ccc(C#N)cn1] |
| 304 | 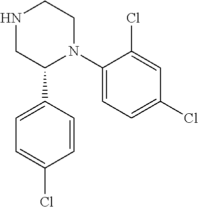 [SMILES: Clc1ccc([C@@H]2CNCCN2c2ccc(Cl)cc2Cl)cc1] | 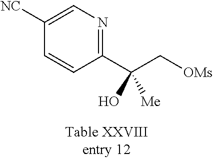 [SMILES: C[C@@](O)(COS(C)(=O)=O)c1ccc(C#N)cn1] | 1022 | 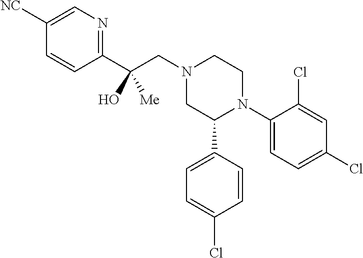 [SMILES: C[C@@](O)(CN1CCN(c2ccc(Cl)cc2Cl)[C@H](c2ccc(Cl)cc2)C1)c1ccc(C#N)cn1] |
| 813b | 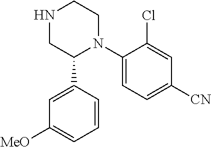 [SMILES: COc1cccc([C@@H]2CNCCN2c2ccc(C#N)cc2Cl)c1] | 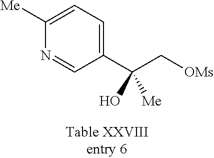 [SMILES: Cc1ccc([C@](C)(O)COS(C)(=O)=O)cn1] | 1023 | 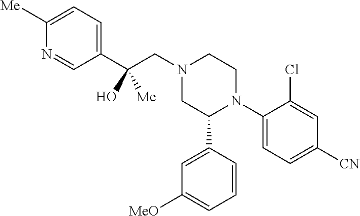 [SMILES: COc1cccc([C@@H]2CN(C[C@@](C)(O)c3ccc(C)nc3)CCN2c2ccc(C#N)cc2Cl)c1] |
| 310 | 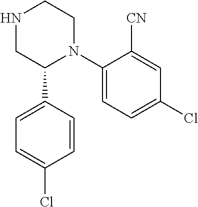 [SMILES: N#Cc1cc(Cl)ccc1N1CCNC[C@H]1c1ccc(Cl)cc1] | 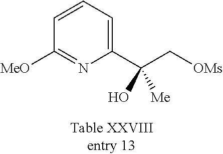 [SMILES: COc1cccc([C@](C)(O)COS(C)(=O)=O)n1] | 1024 | 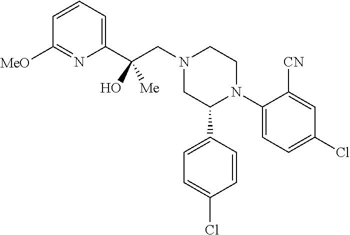 [SMILES: COc1cccc([C@](C)(O)CN2CCN(c3ccc(Cl)cc3C#N)[C@H](c3ccc(Cl)cc3)C2)n1] |
| 305 | 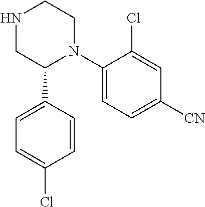 [SMILES: N#Cc1ccc(N2CCNC[C@H]2c2ccc(Cl)cc2)c(Cl)c1] | 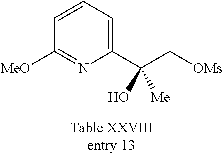 [SMILES: COc1cccc([C@](C)(O)COS(C)(=O)=O)n1] | 1025 | 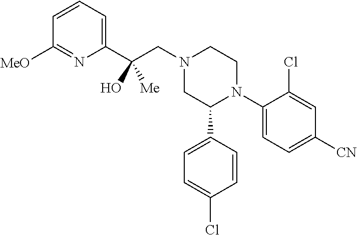 [SMILES: COc1cccc([C@](C)(O)CN2CCN(c3ccc(C#N)cc3Cl)[C@H](c3ccc(Cl)cc3)C2)n1] |
| 822 | 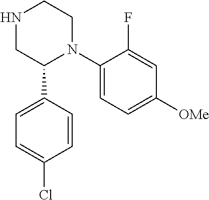 [SMILES: COc1ccc(N2CCNC[C@H]2c2ccc(Cl)cc2)c(F)c1] | 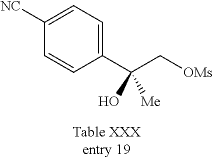 [SMILES: C[C@@](O)(COS(C)(=O)=O)c1ccc(C#N)cc1] | 1026 | 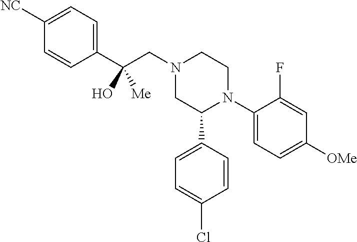 [SMILES: COc1ccc(N2CCN(C[C@@](C)(O)c3ccc(C#N)cc3)C[C@H]2c2ccc(Cl)cc2)c(F)c1] |
| 833 | 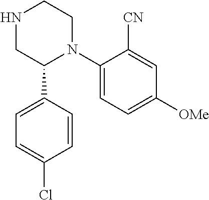 [SMILES: COc1ccc(N2CCNC[C@H]2c2ccc(Cl)cc2)c(C#N)c1] | 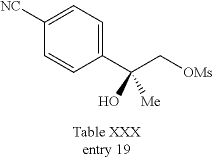 [SMILES: C[C@@](O)(COS(C)(=O)=O)c1ccc(C#N)cc1] | 1027 | 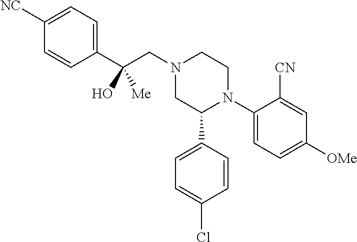 [SMILES: COc1ccc(N2CCN(C[C@@](C)(O)c3ccc(C#N)cc3)C[C@H]2c2ccc(Cl)cc2)c(C#N)c1] |
| 310 | 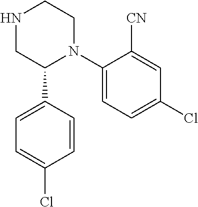 [SMILES: N#Cc1cc(Cl)ccc1N1CCNC[C@H]1c1ccc(Cl)cc1] | 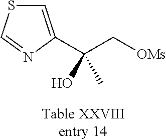 | 1028 | 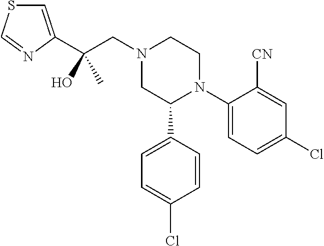 [SMILES: C[C@@](O)(CN1CCN(c2ccc(Cl)cc2C#N)[C@H](c2ccc(Cl)cc2)C1)c1cscn1] |
| 305 | 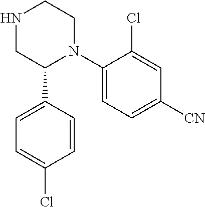 [SMILES: N#Cc1ccc(N2CCNC[C@H]2c2ccc(Cl)cc2)c(Cl)c1] | 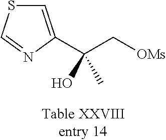 | 1029 | 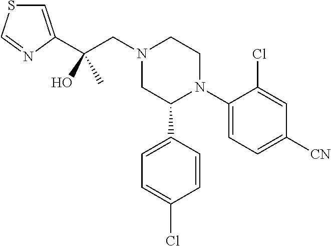 [SMILES: C[C@@](O)(CN1CCN(c2ccc(C#N)cc2Cl)[C@H](c2ccc(Cl)cc2)C1)c1cscn1] |
| 837 | 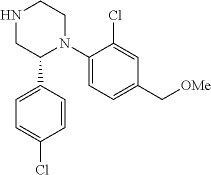 [SMILES: COCc1ccc(N2CCNC[C@H]2c2ccc(Cl)cc2)c(Cl)c1] | 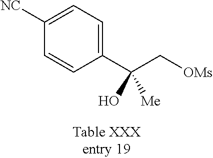 [SMILES: C[C@@](O)(COS(C)(=O)=O)c1ccc(C#N)cc1] | 1030 | 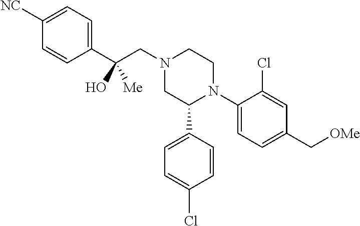 [SMILES: COCc1ccc(N2CCN(C[C@@](C)(O)c3ccc(C#N)cc3)C[C@H]2c2ccc(Cl)cc2)c(Cl)c1] |
| 304 | 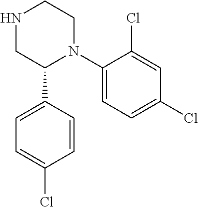 [SMILES: Clc1ccc([C@@H]2CNCCN2c2ccc(Cl)cc2Cl)cc1] | 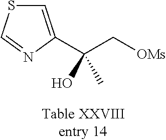 | 1031 | 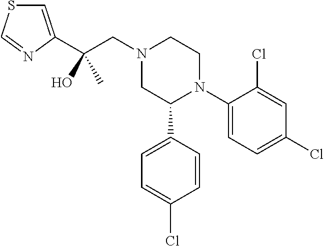 [SMILES: C[C@@](O)(CN1CCN(c2ccc(Cl)cc2Cl)[C@H](c2ccc(Cl)cc2)C1)c1cscn1] |
| 810 | 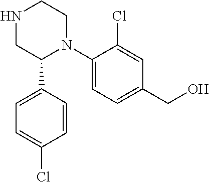 [SMILES: OCc1ccc(N2CCNC[C@H]2c2ccc(Cl)cc2)c(Cl)c1] | 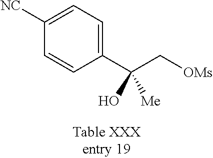 [SMILES: C[C@@](O)(COS(C)(=O)=O)c1ccc(C#N)cc1] | 1032 | 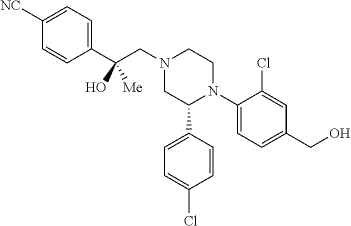 [SMILES: C[C@@](O)(CN1CCN(c2ccc(CO)cc2Cl)[C@H](c2ccc(Cl)cc2)C1)c1ccc(C#N)cc1] |
| 305 | 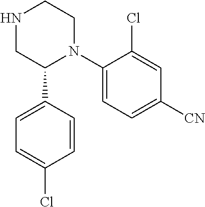 [SMILES: N#Cc1ccc(N2CCNC[C@H]2c2ccc(Cl)cc2)c(Cl)c1] | 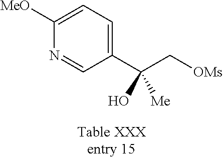 [SMILES: COc1ccc([C@](C)(O)COS(C)(=O)=O)cn1] | 1033 | 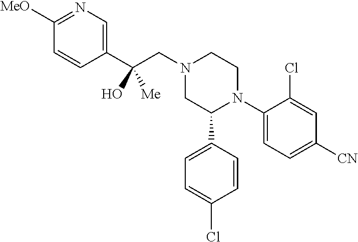 [SMILES: COc1ccc([C@](C)(O)CN2CCN(c3ccc(C#N)cc3Cl)[C@H](c3ccc(Cl)cc3)C2)cn1] |
| 310 | 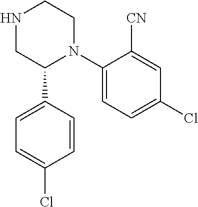 [SMILES: N#Cc1cc(Cl)ccc1N1CCNC[C@H]1c1ccc(Cl)cc1] | 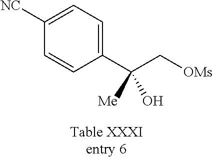 [SMILES: C[C@](O)(COS(C)(=O)=O)c1ccc(C#N)cc1] | 1034 | 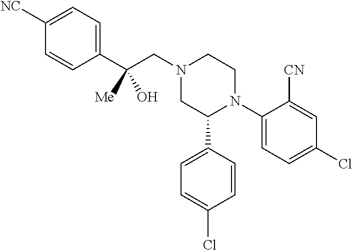 [SMILES: C[C@](O)(CN1CCN(c2ccc(Cl)cc2C#N)[C@H](c2ccc(Cl)cc2)C1)c1ccc(C#N)cc1] |
| 310 | 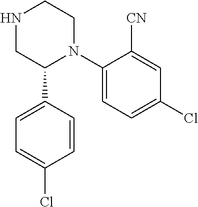 [SMILES: N#Cc1cc(Cl)ccc1N1CCNC[C@H]1c1ccc(Cl)cc1] | 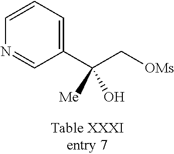 [SMILES: C[C@](O)(COS(C)(=O)=O)c1cccnc1] | 1035 | 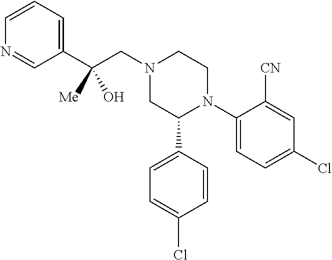 [SMILES: C[C@](O)(CN1CCN(c2ccc(Cl)cc2C#N)[C@H](c2ccc(Cl)cc2)C1)c1cccnc1] |
| 826c | 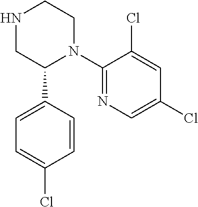 [SMILES: Clc1ccc([C@@H]2CNCCN2c2ncc(Cl)cc2Cl)cc1] | 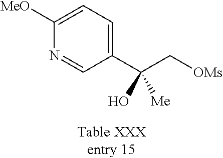 [SMILES: COc1ccc([C@](C)(O)COS(C)(=O)=O)cn1] | 1035a | 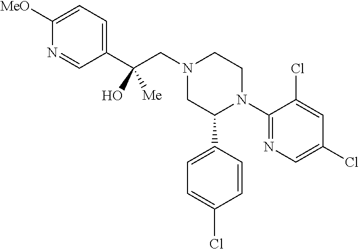 [SMILES: COc1ccc([C@](C)(O)CN2CCN(c3ncc(Cl)cc3Cl)[C@H](c3ccc(Cl)cc3)C2)cn1] |
| 826c | 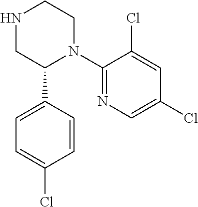 [SMILES: Clc1ccc([C@@H]2CNCCN2c2ncc(Cl)cc2Cl)cc1] | 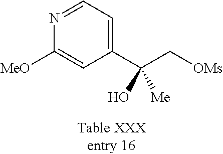 [SMILES: COc1cc([C@](C)(O)COS(C)(=O)=O)ccn1] | 1035b | 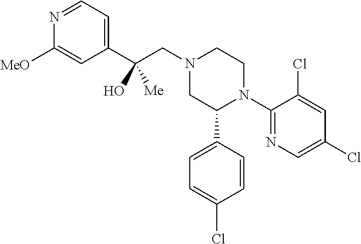 [SMILES: COc1cc([C@](C)(O)CN2CCN(c3ncc(Cl)cc3Cl)[C@H](c3ccc(Cl)cc3)C2)ccn1] |
| 826c | 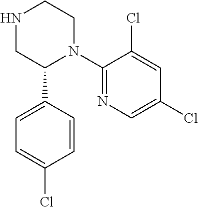 [SMILES: Clc1ccc([C@@H]2CNCCN2c2ncc(Cl)cc2Cl)cc1] | 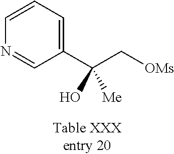 [SMILES: C[C@@](O)(COS(C)(=O)=O)c1cccnc1] | 1035c | 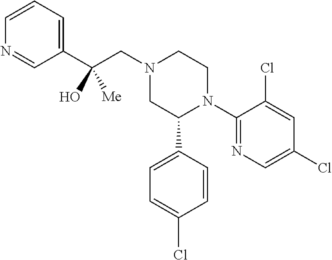 [SMILES: C[C@@](O)(CN1CCN(c2ncc(Cl)cc2Cl)[C@H](c2ccc(Cl)cc2)C1)c1cccnc1] |
| 304 | 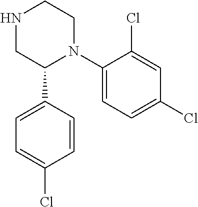 [SMILES: Clc1ccc([C@@H]2CNCCN2c2ccc(Cl)cc2Cl)cc1] | 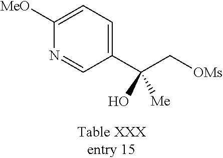 [SMILES: COc1ccc([C@](C)(O)COS(C)(=O)=O)cn1] | 1035d | 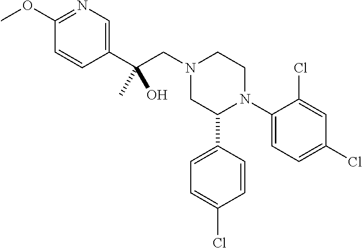 [SMILES: COc1ccc([C@](C)(O)CN2CCN(c3ccc(Cl)cc3Cl)[C@H](c3ccc(Cl)cc3)C2)cn1] |
| 304 | 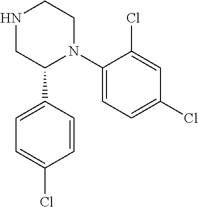 [SMILES: Clc1ccc([C@@H]2CNCCN2c2ccc(Cl)cc2Cl)cc1] | 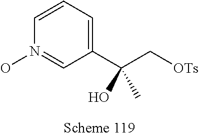 | 1035e | 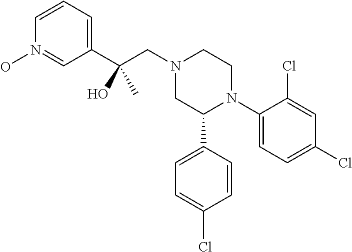 |
| 305 | 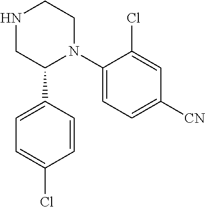 [SMILES: N#Cc1ccc(N2CCNC[C@H]2c2ccc(Cl)cc2)c(Cl)c1] | 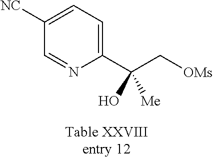 [SMILES: C[C@@](O)(COS(C)(=O)=O)c1ccc(C#N)cn1] | 1035f | 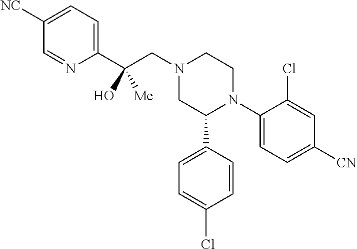 [SMILES: C[C@@](O)(CN1CCN(c2ccc(C#N)cc2Cl)[C@H](c2ccc(Cl)cc2)C1)c1ccc(C#N)cn1] |
| 826 | 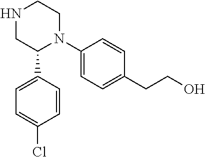 [SMILES: OCCc1ccc(N2CCNC[C@H]2c2ccc(Cl)cc2)cc1] | 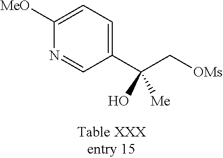 [SMILES: COc1ccc([C@](C)(O)COS(C)(=O)=O)cn1] | 1035g | 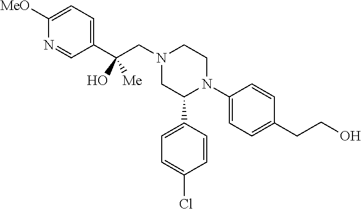 [SMILES: COc1ccc([C@](C)(O)CN2CCN(c3ccc(CCO)cc3)[C@H](c3ccc(Cl)cc3)C2)cn1] |
| 813a | 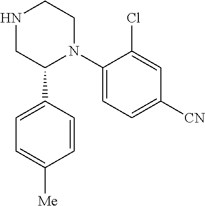 [SMILES: Cc1ccc([C@@H]2CNCCN2c2ccc(C#N)cc2Cl)cc1] | 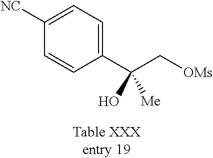 [SMILES: C[C@@](O)(COS(C)(=O)=O)c1ccc(C#N)cc1] | 1035h | 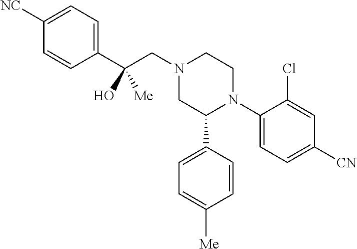 [SMILES: Cc1ccc([C@@H]2CN(C[C@@](C)(O)c3ccc(C#N)cc3)CCN2c2ccc(C#N)cc2Cl)cc1] |
| 826 | 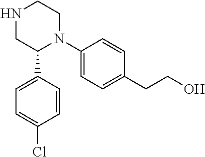 [SMILES: OCCc1ccc(N2CCNC[C@H]2c2ccc(Cl)cc2)cc1] | 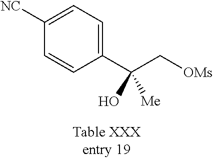 [SMILES: C[C@@](O)(COS(C)(=O)=O)c1ccc(C#N)cc1] | 1035i | 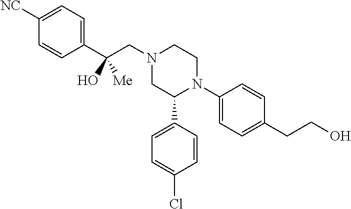 [SMILES: C[C@@](O)(CN1CCN(c2ccc(CCO)cc2)[C@H](c2ccc(Cl)cc2)C1)c1ccc(C#N)cc1] |

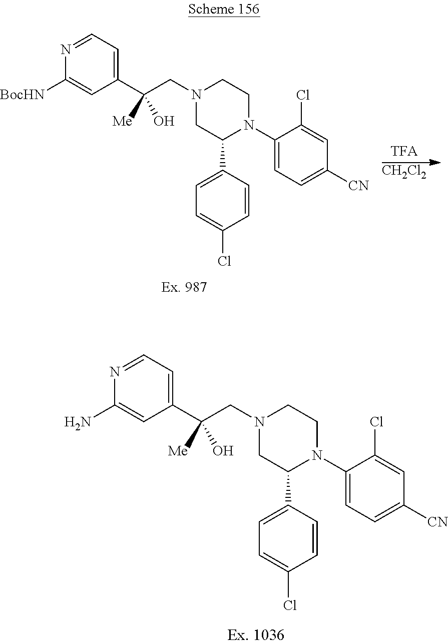

To a solution of Example 987 (200 mg) in CH₂Cl₂ (10 mL) was added TFA (5 mL). The resultant solution was stirred at RT for 4 hours. The solution was concentrated. The residue was partitioned between CH₂Cl₂ and NaHCO_{3(aq.)}. The aqueous layer was extracted with CH₂Cl₂ (3×). The combined organic layers were dried over Na₂SO₄, filtered and concentrated to afford Example 1036.

The following examples were prepared via a method similar to that described in Scheme 156.

TABLE XXXVII

| Ex. | Carbamate | Ex. | Amine |
|---|---|---|---|
| 974 | 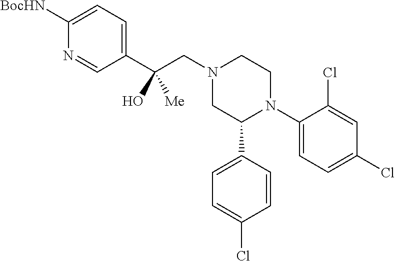 [SMILES: CC(C)(C)OC(=O)Nc1ccc([C@](C)(O)CN2CCN(c3ccc(Cl)cc3Cl)[C@H](c3ccc(Cl)cc3)C2)cn1] | 1037 | 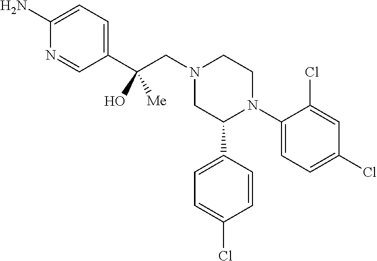 [SMILES: C[C@@](O)(CN1CCN(c2ccc(Cl)cc2Cl)[C@H](c2ccc(Cl)cc2)C1)c1ccc(N)nc1] |
| 975 | 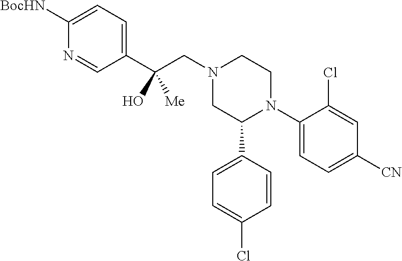 [SMILES: CC(C)(C)OC(=O)Nc1ccc([C@](C)(O)CN2CCN(c3ccc(C#N)cc3Cl)[C@H](c3ccc(Cl)cc3)C2)cn1] | 1038 | 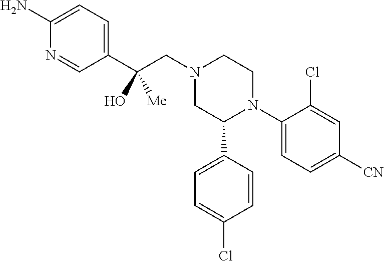 [SMILES: C[C@@](O)(CN1CCN(c2ccc(C#N)cc2Cl)[C@H](c2ccc(Cl)cc2)C1)c1ccc(N)nc1] |
| 986 | 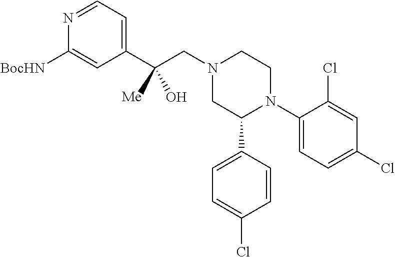 [SMILES: CC(C)(C)OC(=O)Nc1cc([C@@](C)(O)CN2CCN(c3ccc(Cl)cc3Cl)[C@H](c3ccc(Cl)cc3)C2)ccn1] | 1039 | 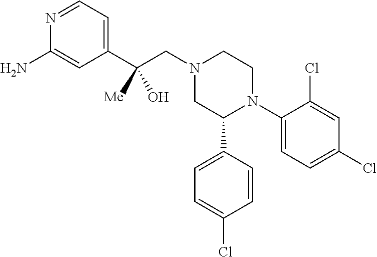 [SMILES: C[C@](O)(CN1CCN(c2ccc(Cl)cc2Cl)[C@H](c2ccc(Cl)cc2)C1)c1ccnc(N)c1] |

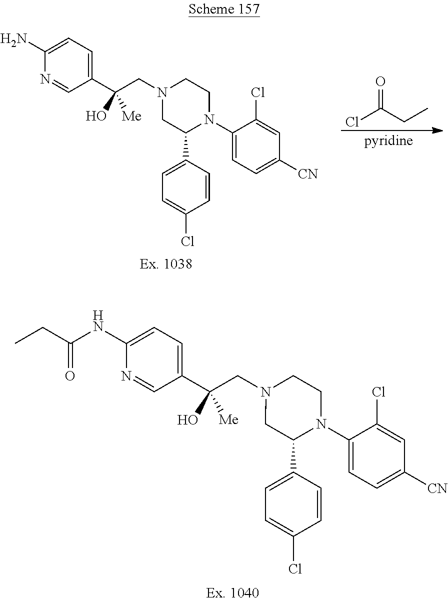

To a solution of Example 1038 (68 mg, 0.14 mmol) in pyridine (2 mL) was added propionyl chloride (14 mg, 0.15 mmol). The resultant solution was stirred at RT for 3 days. The solution was concentrated. The residue was partitioned between EtOAc and water. The aqueous layer was extracted with EtOAc (3×). The combined organic layers were washed with brine, dried over Na₂SO₄, filtered and concentrated. The crude product was purified via flash chromatography (SiO₂: gradient elution 100:0 to 30:70 hexanes:EtOAc) to afford Example 1040 (36 mg) as a clear oil.

The following examples were prepared via a method similar to that described in Scheme 157.

TABLE XXXVII

| | | Acid | | |
|---|---|---|---|---|
| | | chloride | | |
| | | or | | |
| Ex. | Amine | anhydride | Ex. | Structure |
| 1038 | 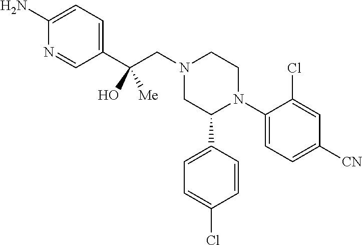 [SMILES: C[C@@](O)(CN1CCN(c2ccc(C#N)cc2Cl)[C@H](c2ccc(Cl)cc2)C1)c1ccc(N)nc1] | Ac₂O | 1041 | 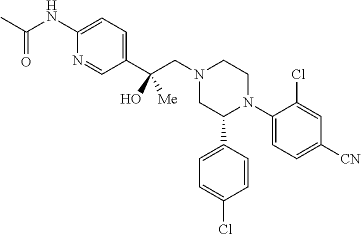 [SMILES: CC(=O)Nc1ccc([C@](C)(O)CN2CCN(c3ccc(C#N)cc3Cl)[C@H](c3ccc(Cl)cc3)C2)cn1] |
| 1038 | 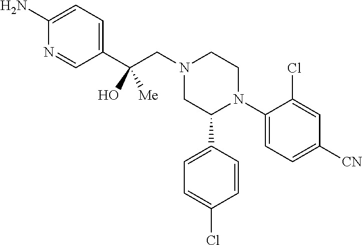 [SMILES: C[C@@](O)(CN1CCN(c2ccc(C#N)cc2Cl)[C@H](c2ccc(Cl)cc2)C1)c1ccc(N)nc1] | 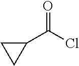 | 1042 | 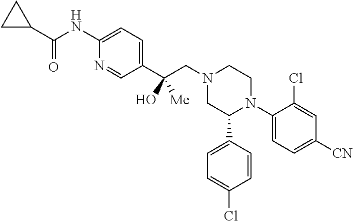 [SMILES: C[C@@](O)(CN1CCN(c2ccc(C#N)cc2Cl)[C@H](c2ccc(Cl)cc2)C1)c1ccc(NC(=O)C2CC2)nc1] |
| 1038 | 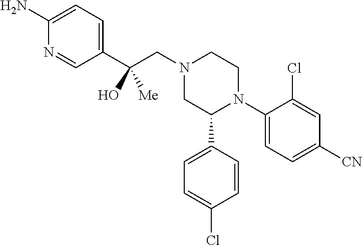 [SMILES: C[C@@](O)(CN1CCN(c2ccc(C#N)cc2Cl)[C@H](c2ccc(Cl)cc2)C1)c1ccc(N)nc1] | 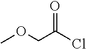 | 1043 | 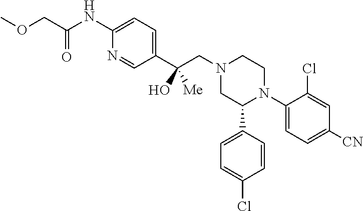 [SMILES: COCC(=O)Nc1ccc([C@](C)(O)CN2CCN(c3ccc(C#N)cc3Cl)[C@H](c3ccc(Cl)cc3)C2)cn1] |
| 1036 | 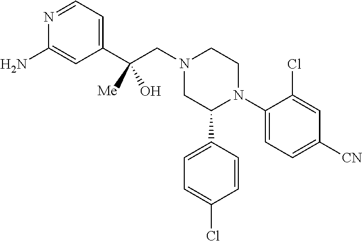 [SMILES: C[C@](O)(CN1CCN(c2ccc(C#N)cc2Cl)[C@H](c2ccc(Cl)cc2)C1)c1ccnc(N)c1] | 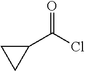 | 1044 | 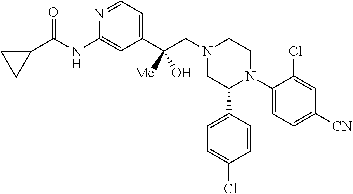 [SMILES: C[C@](O)(CN1CCN(c2ccc(C#N)cc2Cl)[C@H](c2ccc(Cl)cc2)C1)c1ccnc(NC(=O)C2CC2)c1] |
| 1039 | 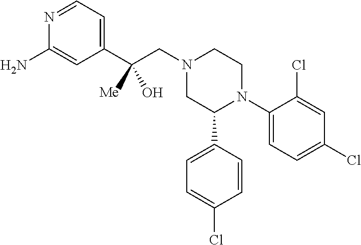 [SMILES: C[C@](O)(CN1CCN(c2ccc(Cl)cc2Cl)[C@H](c2ccc(Cl)cc2)C1)c1ccnc(N)c1] | 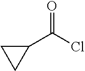 | 1045 | 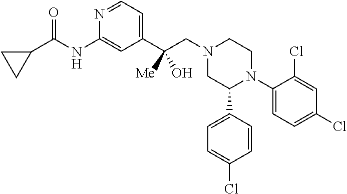 [SMILES: C[C@](O)(CN1CCN(c2ccc(Cl)cc2Cl)[C@H](c2ccc(Cl)cc2)C1)c1ccnc(NC(=O)C2CC2)c1] |
| 1039 | 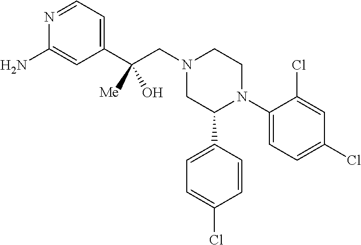 [SMILES: C[C@](O)(CN1CCN(c2ccc(Cl)cc2Cl)[C@H](c2ccc(Cl)cc2)C1)c1ccnc(N)c1] | 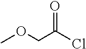 | 1046 | 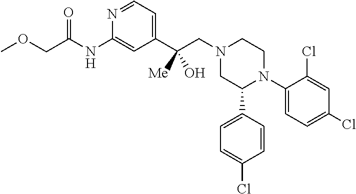 [SMILES: COCC(=O)Nc1cc([C@@](C)(O)CN2CCN(c3ccc(Cl)cc3Cl)[C@H](c3ccc(Cl)cc3)C2)ccn1] |

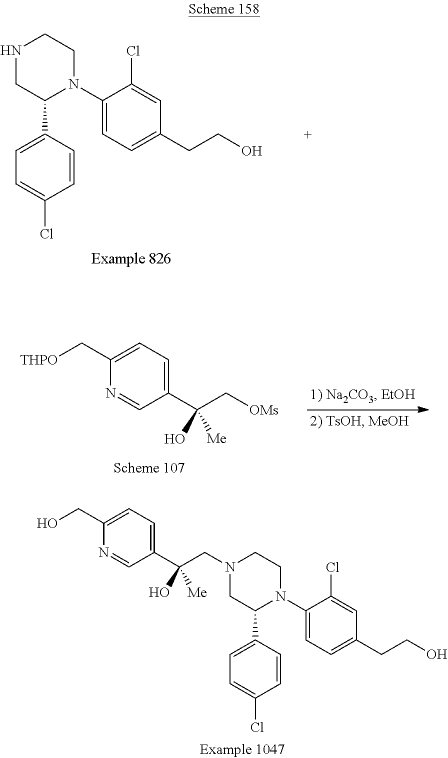

To a pressure tube containing Example 826 (111 mg, 0.35 mmol) in EtOH (2 mL) was added the mesylate from Scheme 107 (145 mg, 0.42 mmol) and Na₂CO₃ (48 mg, 0.46 mmol). The tube was sealed and the mixture was heated to 80° C. with stirring for 1.5 days. The mixture was cooled to RT and transferred to a round bottom flask. The solvent was removed in vacuo. The residue was partitioned between EtOAc and water. The aqueous layer was extracted with EtOAc (3×). The combined organic layers were washed with brine, dried over Na₂SO₄, filtered and concentrated. The crude product was purified via flash chromatography (SiO₂: gradient elution 100:0 to 1:1 hexanes:EtOAc) to afford the intermediate (130 mg).

This intermediate (130 mg, 0.23 mmol) was dissolved in MeOH (10 mL). To the solution was added TsOH.H₂O (55 mg, 0.29 mmol). The resultant solution was stirred at RT overnight. The solution was concentrated in vacuo. The residue was partitioned between EtOAc and 1 N NaOH (aq.). The aqueous layer was extracted with EtOAc (3×). The combined organic layers were washed with brine, dried over Na₂SO₄, filtered and concentrated. The crude product was purified via flash chromatography [SiO₂: gradient elution 100:0:0 to 95:5:1 CH₂Cl₂:MeOH:7 N NH₃ (MeOH)] to afford Example 1047 (77 mg) as a white foam.

The following examples were prepared using a method similar to that described in Scheme 158.

TABLE XXXIX

| | Piperazine | | | |
|---|---|---|---|---|
| Ex. | Core | Mesylate | Ex. | Structure |
| 304 | 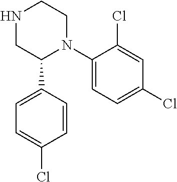 [SMILES: Clc1ccc([C@@H]2CNCCN2c2ccc(Cl)cc2Cl)cc1] | 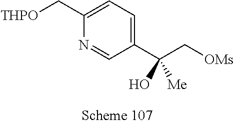 | 1048 | 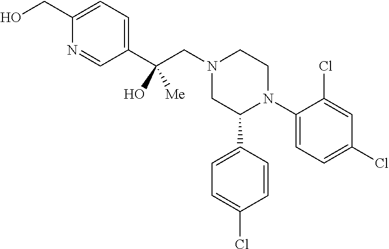 [SMILES: C[C@@](O)(CN1CCN(c2ccc(Cl)cc2Cl)[C@H](c2ccc(Cl)cc2)C1)c1ccc(CO)nc1] |
| 305 | 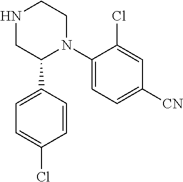 [SMILES: N#Cc1ccc(N2CCNC[C@H]2c2ccc(Cl)cc2)c(Cl)c1] | 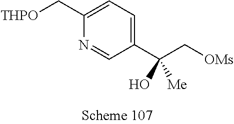 | 1049 | 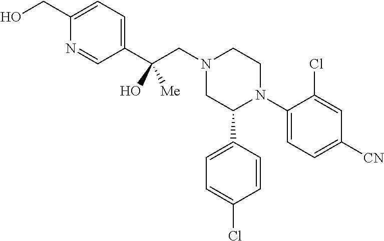 [SMILES: C[C@@](O)(CN1CCN(c2ccc(C#N)cc2Cl)[C@H](c2ccc(Cl)cc2)C1)c1ccc(CO)nc1] |
| 305 | 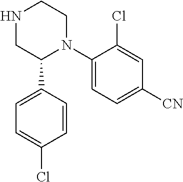 [SMILES: N#Cc1ccc(N2CCNC[C@H]2c2ccc(Cl)cc2)c(Cl)c1] | 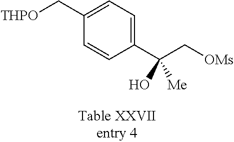 | 1050 | 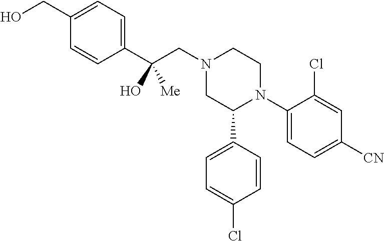 [SMILES: C[C@@](O)(CN1CCN(c2ccc(C#N)cc2Cl)[C@H](c2ccc(Cl)cc2)C1)c1ccc(CO)cc1] |
| 310 | 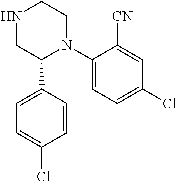 [SMILES: N#Cc1cc(Cl)ccc1N1CCNC[C@H]1c1ccc(Cl)cc1] | 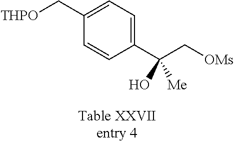 | 1051 | 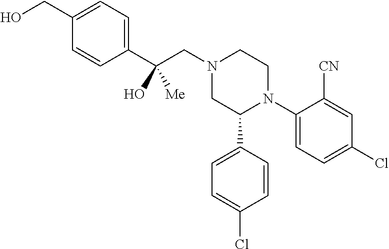 [SMILES: C[C@@](O)(CN1CCN(c2ccc(Cl)cc2C#N)[C@H](c2ccc(Cl)cc2)C1)c1ccc(CO)cc1] |
| 813b | 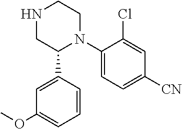 [SMILES: COc1cccc([C@@H]2CNCCN2c2ccc(C#N)cc2Cl)c1] | 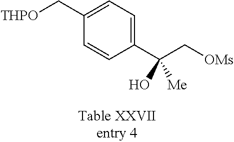 | 1052 | 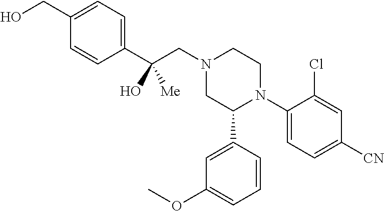 [SMILES: COc1cccc([C@@H]2CN(C[C@@](C)(O)c3ccc(CO)cc3)CCN2c2ccc(C#N)cc2Cl)c1] |
| 813b | 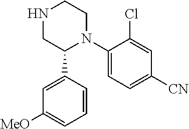 [SMILES: COc1cccc([C@@H]2CNCCN2c2ccc(C#N)cc2Cl)c1] | 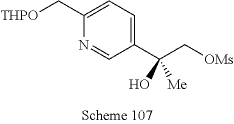 | 1053 | 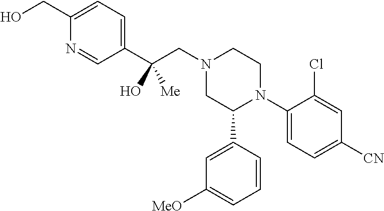 [SMILES: COc1cccc([C@@H]2CN(C[C@@](C)(O)c3ccc(CO)nc3)CCN2c2ccc(C#N)cc2Cl)c1] |
| 305 | 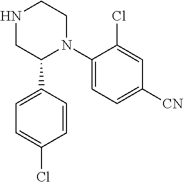 [SMILES: N#Cc1ccc(N2CCNC[C@H]2c2ccc(Cl)cc2)c(Cl)c1] | 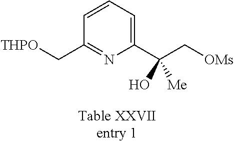 | 1054 | 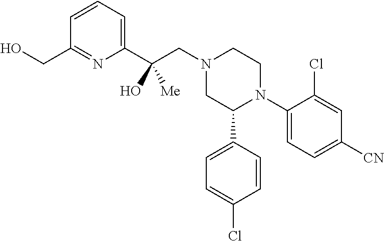 [SMILES: C[C@@](O)(CN1CCN(c2ccc(C#N)cc2Cl)[C@H](c2ccc(Cl)cc2)C1)c1cccc(CO)n1] |
| 310 | 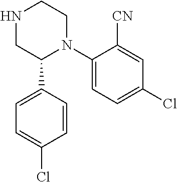 [SMILES: N#Cc1cc(Cl)ccc1N1CCNC[C@H]1c1ccc(Cl)cc1] | 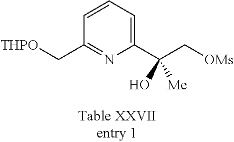 | 1055 | 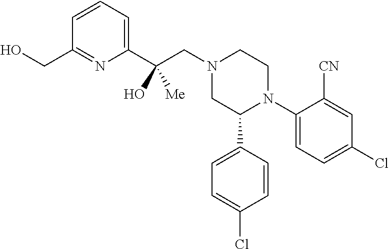 [SMILES: C[C@@](O)(CN1CCN(c2ccc(Cl)cc2C#N)[C@H](c2ccc(Cl)cc2)C1)c1cccc(CO)n1] |
| 822 | 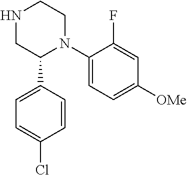 [SMILES: COc1ccc(N2CCNC[C@H]2c2ccc(Cl)cc2)c(F)c1] | 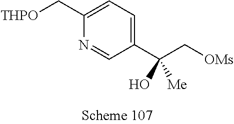 | 1056 | 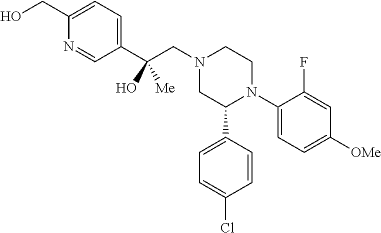 [SMILES: COc1ccc(N2CCN(C[C@@](C)(O)c3ccc(CO)nc3)C[C@H]2c2ccc(Cl)cc2)c(F)c1] |
| 833 | 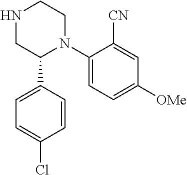 [SMILES: COc1ccc(N2CCNC[C@H]2c2ccc(Cl)cc2)c(C#N)c1] | 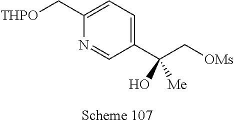 | 1057 | 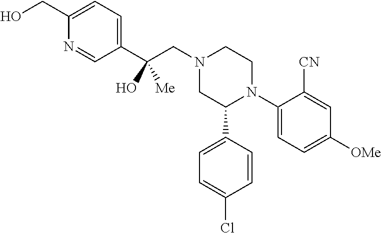 [SMILES: COc1ccc(N2CCN(C[C@@](C)(O)c3ccc(CO)nc3)C[C@H]2c2ccc(Cl)cc2)c(C#N)c1] |
| 833 | 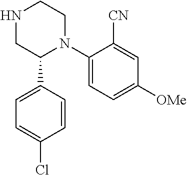 [SMILES: COc1ccc(N2CCNC[C@H]2c2ccc(Cl)cc2)c(C#N)c1] | 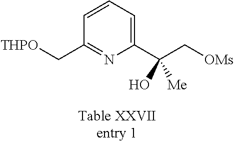 | 1058 | 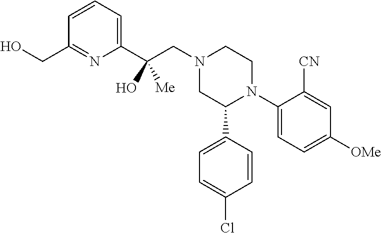 [SMILES: COc1ccc(N2CCN(C[C@@](C)(O)c3cccc(CO)n3)C[C@H]2c2ccc(Cl)cc2)c(C#N)c1] |
| 833 | 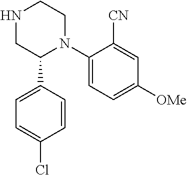 [SMILES: COc1ccc(N2CCNC[C@H]2c2ccc(Cl)cc2)c(C#N)c1] | 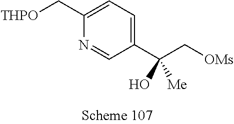 | 1059 | 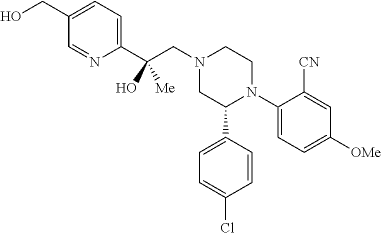 [SMILES: COc1ccc(N2CCN(C[C@@](C)(O)c3ccc(CO)cn3)C[C@H]2c2ccc(Cl)cc2)c(C#N)c1] |
| 813 | 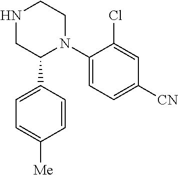 [SMILES: Cc1ccc([C@@H]2CNCCN2c2ccc(C#N)cc2Cl)cc1] | 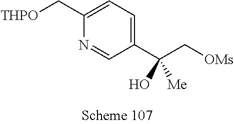 | 1060 | 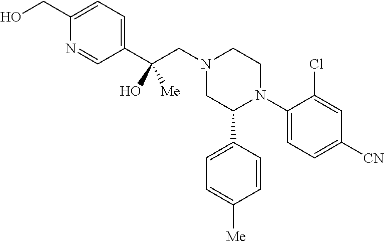 [SMILES: Cc1ccc([C@@H]2CN(C[C@@](C)(O)c3ccc(CO)nc3)CCN2c2ccc(C#N)cc2Cl)cc1] |
| 304 | 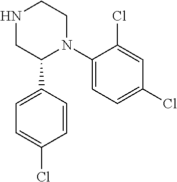 [SMILES: Clc1ccc([C@@H]2CNCCN2c2ccc(Cl)cc2Cl)cc1] | 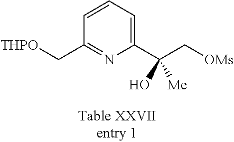 | 1061 | 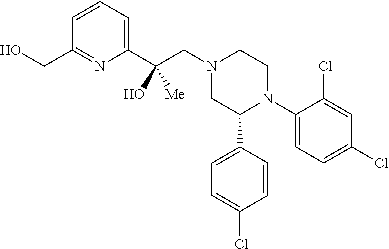 [SMILES: C[C@@](O)(CN1CCN(c2ccc(Cl)cc2Cl)[C@H](c2ccc(Cl)cc2)C1)c1cccc(CO)n1] |
| 310 | 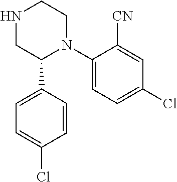 [SMILES: N#Cc1cc(Cl)ccc1N1CCNC[C@H]1c1ccc(Cl)cc1] | 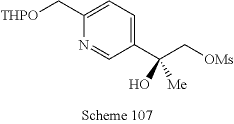 | 1062a | 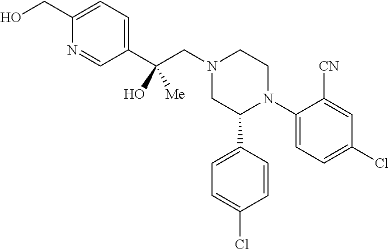 [SMILES: C[C@@](O)(CN1CCN(c2ccc(Cl)cc2C#N)[C@H](c2ccc(Cl)cc2)C1)c1ccc(CO)nc1] |
| 826d | 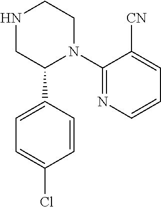 [SMILES: N#Cc1cccnc1N1CCNC[C@H]1c1ccc(Cl)cc1] | 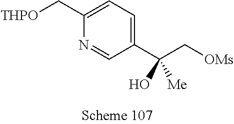 | 1062b | 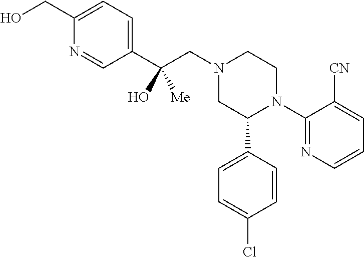 [SMILES: C[C@@](O)(CN1CCN(c2ncccc2C#N)[C@H](c2ccc(Cl)cc2)C1)c1ccc(CO)nc1] |
| 826 | 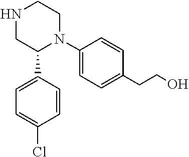 [SMILES: OCCc1ccc(N2CCNC[C@H]2c2ccc(Cl)cc2)cc1] | 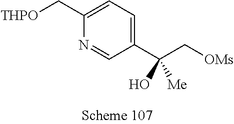 | 1062c | 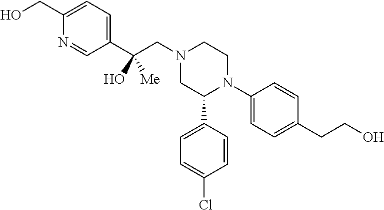 [SMILES: C[C@@](O)(CN1CCN(c2ccc(CCO)cc2)[C@H](c2ccc(Cl)cc2)C1)c1ccc(CO)nc1] |

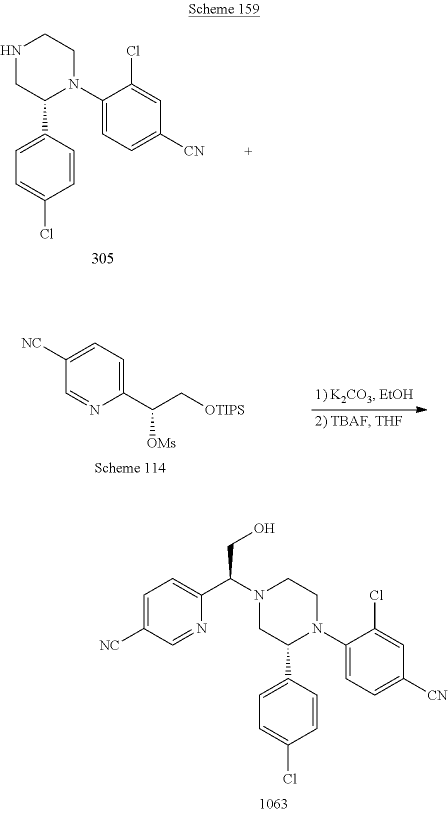

### Step 1

To a pressure tube containing the mesylate from Scheme 114 (120 mg, 0.30 mmol) and Example 305 (83 mg, 0.25 mmol) was added K₂CO₃ (45 mg, 0.33 mmol) and MeCN (2 mL). The tube was sealed and the mixture was heated to 85° C. with stirring for 1.5 days. The mixture was concentrated. The residue was partitioned between EtOAc and water. The aqueous layer was extracted with EtOAc (3×). The combined organic layers were washed with brine, dried over Na₂SO₄, filtered and concentrated. The crude residue was purified via flash chromatography (SiO₂: gradient elution 100:0 to 3:1 hexanes:EtOAc) to afford the silyl alcohol intermediate (110 mg, 0.17 mmol).

### Step 2

The material from step 1 was dissolved in THF (5 mL). To this solution was added a solution of TBAF (1M in THF, 0.23 mL, 0.23 mmol). The resultant solution was stirred at RT overnight. The solution was transferred to a flash column and purified via flash chromatography (SiO₂: gradient elution 100:0 to 30:70 hexanes:EtOAc) to afford Example 1063 (20 mg).

TABLE XL

| | Piperazine | | | |
|---|---|---|---|---|
| Ex. | Core | mesylate | Ex. | Structure |
| 304 | 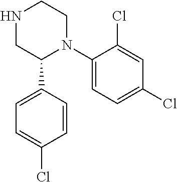 [SMILES: Clc1ccc([C@@H]2CNCCN2c2ccc(Cl)cc2Cl)cc1] | 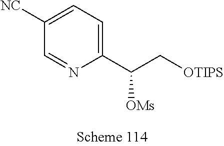 | 1064 | 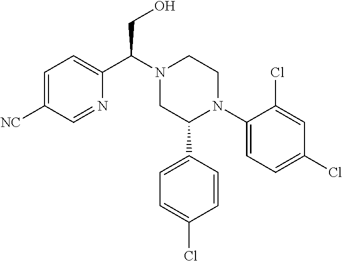 [SMILES: N#Cc1ccc([C@@H](CO)N2CCN(c3ccc(Cl)cc3Cl)[C@H](c3ccc(Cl)cc3)C2)nc1] |
| 310 | 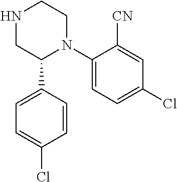 [SMILES: N#Cc1cc(Cl)ccc1N1CCNC[C@H]1c1ccc(Cl)cc1] | 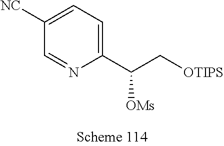 | 1065 | 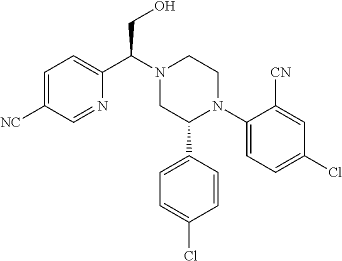 [SMILES: N#Cc1ccc([C@@H](CO)N2CCN(c3ccc(Cl)cc3C#N)[C@H](c3ccc(Cl)cc3)C2)nc1] |
| 822 | 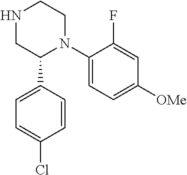 [SMILES: COc1ccc(N2CCNC[C@H]2c2ccc(Cl)cc2)c(F)c1] | 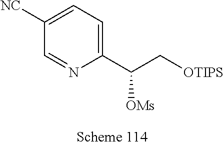 | 1066 | 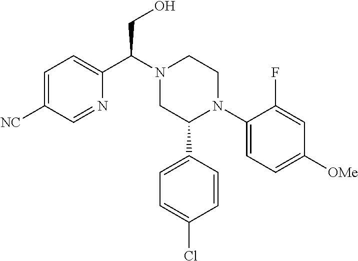 [SMILES: COc1ccc(N2CCN([C@H](CO)c3ccc(C#N)cn3)C[C@H]2c2ccc(Cl)cc2)c(F)c1] |
| 826 | 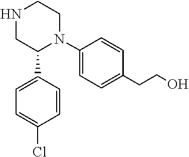 [SMILES: OCCc1ccc(N2CCNC[C@H]2c2ccc(Cl)cc2)cc1] | 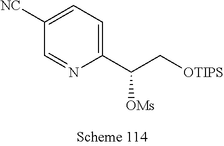 | 1067 | 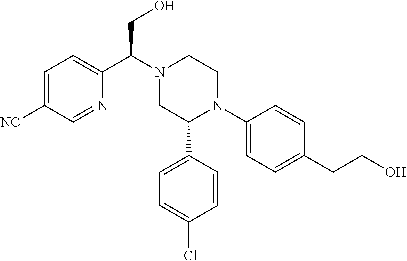 [SMILES: N#Cc1ccc([C@@H](CO)N2CCN(c3ccc(CCO)cc3)[C@H](c3ccc(Cl)cc3)C2)nc1] |

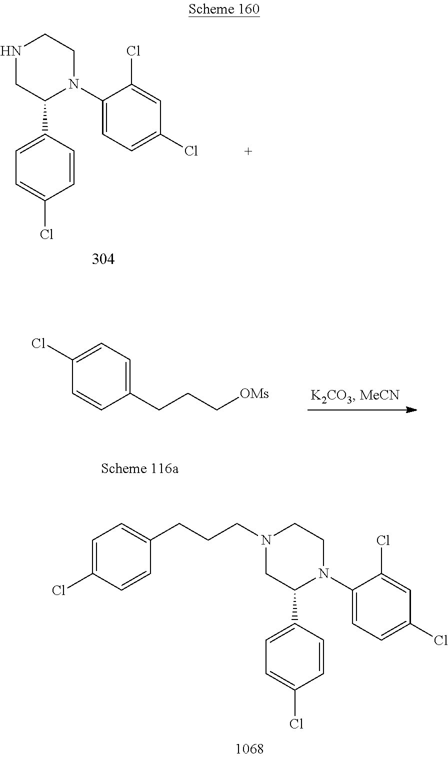 [SMILES: Clc1ccc([C@@H]2CNCCN2c2ccc(Cl)cc2Cl)cc1 ; CS(=O)(=O)OCCCc1ccc(Cl)cc1 ; Clc1ccc(CCCN2CCN(c3ccc(Cl)cc3Cl)[C@H](c3ccc(Cl)cc3)C2)cc1]

Example 1068 was prepared from Example 304 and the mesylate prepared in Scheme 116a using a method similar to that described in Scheme 66 step 3.

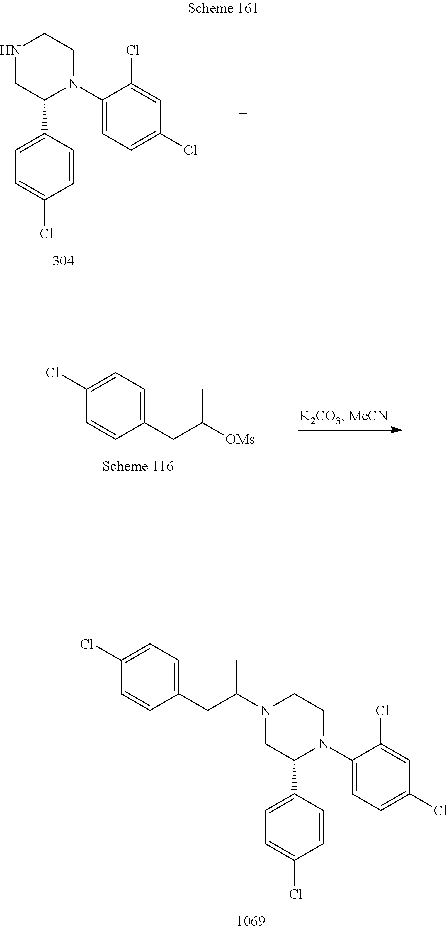 [SMILES: Clc1ccc([C@@H]2CNCCN2c2ccc(Cl)cc2Cl)cc1 ; CC(Cc1ccc(Cl)cc1)OS(C)(=O)=O ; CC(Cc1ccc(Cl)cc1)N1CCN(c2ccc(Cl)cc2Cl)[C@H](c2ccc(Cl)cc2)C1]

Example 1069 was prepared from Example 304 and the mesylate prepared in Scheme 116 using a method similar to that described in Scheme 66 step 3.

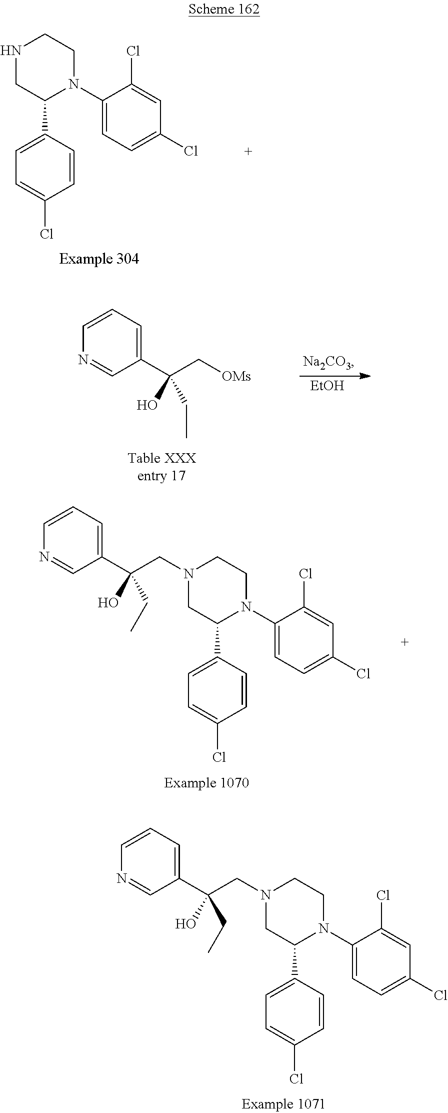 [SMILES: "EtOH" ; Clc1ccc([C@@H]2CNCCN2c2ccc(Cl)cc2Cl)cc1 ; CC[C@@](O)(COS(C)(=O)=O)c1cccnc1 ; CC[C@@](O)(CN1CCN(c2ccc(Cl)cc2Cl)[C@H](c2ccc(Cl)cc2)C1)c1cccnc1 ; CC[C@](O)(CN1CCN(c2ccc(Cl)cc2Cl)[C@H](c2ccc(Cl)cc2)C1)c1cccnc1]

To a pressure tube containing a solution of Example 304 (100 mg, 0.29 mmol) and the mesylate from table XXX entry 17 (94 mg, 0.38 mmol) in EtOH (3 mL) was added Na₂CO₃ (40 mg, 0.38 mmol). The tube was sealed and the mixture was heated to 80° C. with stirring for 16 hours. The solution was concentrated and purified via flash chromatography (SiO₂: gradient elution 100:0:0 to 95:5:0.5 CH₂Cl₂:MeOH: conc NH₄OH_{(aq.)}) to afford a mixture of the diastereomers. These diastereomers were separated via chiral HPLC (Chiralcel AD, 85:15 hexanes:iPrOH) to afford Example 1070 (35 mg) as the faster eluting isomer followed by Example 1071 (17 mg).

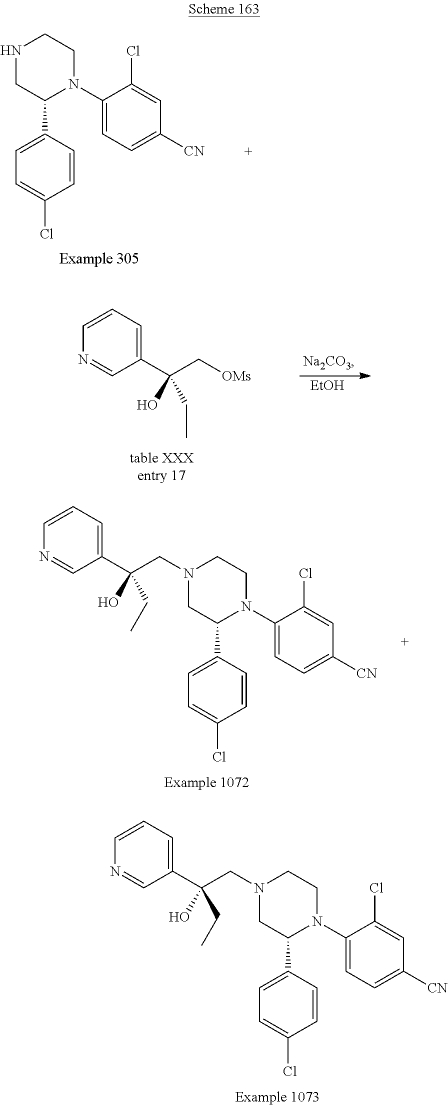 [SMILES: "EtOH" ; N#Cc1ccc(N2CCNC[C@H]2c2ccc(Cl)cc2)c(Cl)c1 ; CC[C@@](O)(COS(C)(=O)=O)c1cccnc1 ; CC[C@@](O)(CN1CCN(c2ccc(C#N)cc2Cl)[C@H](c2ccc(Cl)cc2)C1)c1cccnc1 ; CC[C@](O)(CN1CCN(c2ccc(C#N)cc2Cl)[C@H](c2ccc(Cl)cc2)C1)c1cccnc1]

Example 1072 and Example 1073 were prepared from Example 305 using a method similar to that described in Scheme 162.

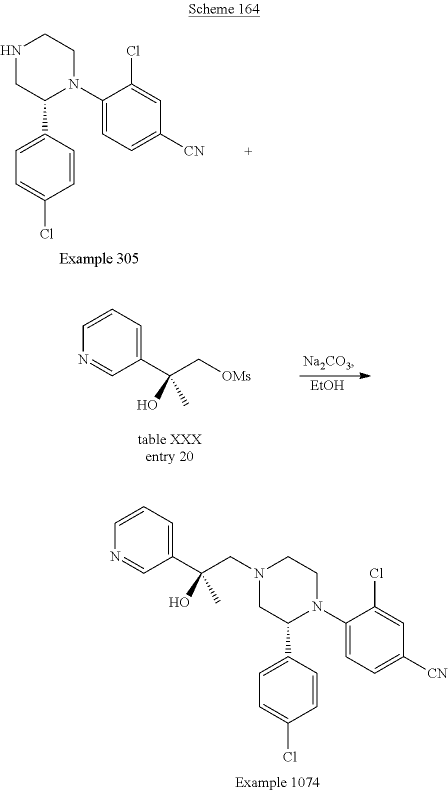 [SMILES: "EtOH" ; N#Cc1ccc(N2CCNC[C@H]2c2ccc(Cl)cc2)c(Cl)c1 ; C[C@@](O)(COS(C)(=O)=O)c1cccnc1 ; C[C@@](O)(CN1CCN(c2ccc(C#N)cc2Cl)[C@H](c2ccc(Cl)cc2)C1)c1cccnc1]

Example 1074 was prepared from Example 305 and the mesylate from Table XXX entry 20 using a method similar to that described in Scheme 162.

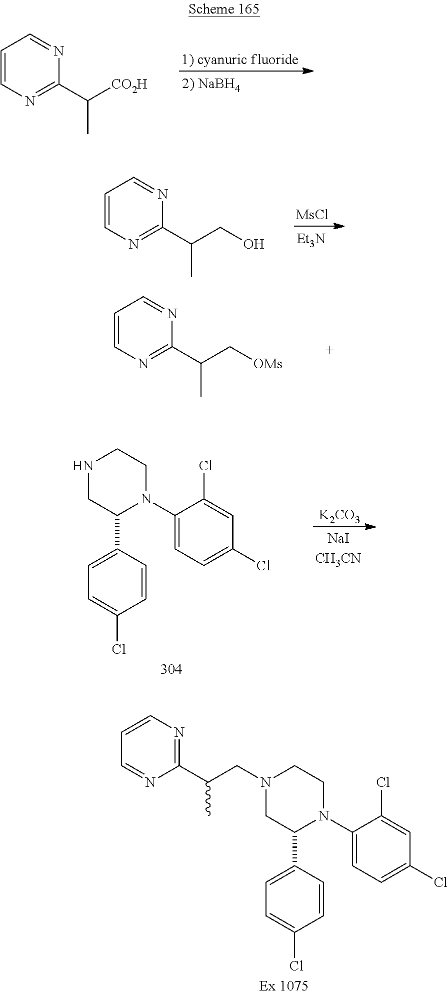

### Step 1

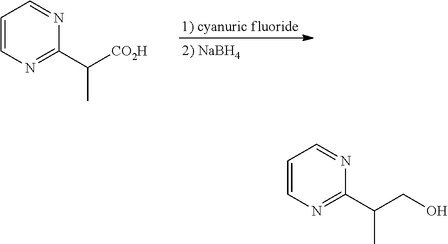

The acid (540 mg, 3.5 mmol) and pyridine (360 mg) were taken up in DCM (10 ml) at 0° C. Cyanuric fluoride (0.63 ml) was added drop-wise to the solution, and the solution was stirred at 0° C. for 4 h. The solution was diluted with DCM and washed with sat. NaHCO_{3(aq.)}. The aqueous layer was extracted with DCM. The combined organic layers were dried (MgSO₄), filtered, and concentrated to afford the acid fluoride as a yellow oil. The crude acid fluoride and NaBH₄ (270 mg) were taken up in DCM (10 ml) at 0° C. Methanol (2 ml) was added slowly to the solution at 0° C., and the resulting solution was warmed to room temperature and stirred at that temperature for 2 h. The solution was partitioned between EtOAc and sat. NH₄Cl_{(aq.)}. The aqueous layer was extracted with EtOAc. The combined organic layers were washed with brine and dried (MgSO₄). The mixture was filtered and concentrated to afford a yellow oil. Purification via preparative thin-layer chromatography (10% MeOH in DCM, SiO₂) provided 135 mg of the alcohol as a color-less oil.

### Step 2

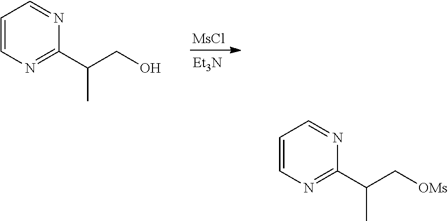

The alcohol (135 mg, 0.98 mmol) and Et₃N (200 mg) were taken up in DCM (15 ml). Methanesulfonyl chloride (167 mg) was added, and the solution was stirred at room temperature for 15 minutes. The solution was diluted with DCM and washed with sat. NaHCO_{3(aq)}. The aqueous layer was extracted with DCM. The combined organic layers were dried (MgSO₄), filtered, and concentrated to afford the mesylate as a yellow oil (195 mg).

### Step 3

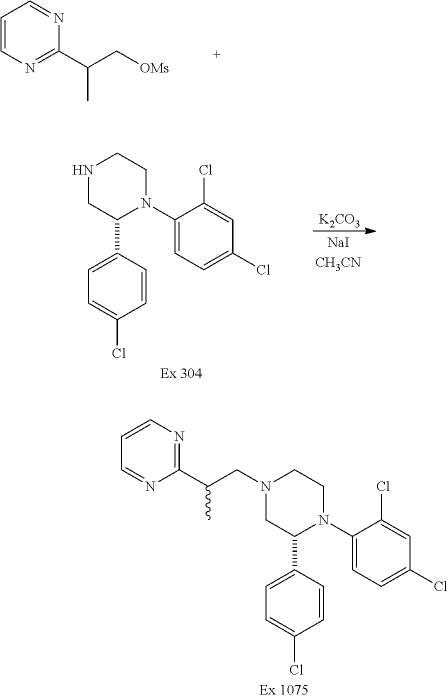

### Step 3

The mesylate (140 mg), piperazine (160 mg), K₂CO₃ (200 mg), and NaI (10 mg) were taken up in CH₃CN (25 ml). The mixture was heated at reflux for 18 h (85° C.). The mixture was partitioned between EtOAc and water. The aqueous layer was extracted with EtOAc. The combined organic layers were washed with brine and dried (MgSO₄). Filtration and concentration gave a yellow oil. Purification via preparative thin-layer chromatography (5% MeOH in DCM, SiO₂) gave 19 mg of Example 1075 as a colorless oil.

### Preparation of Example 1076

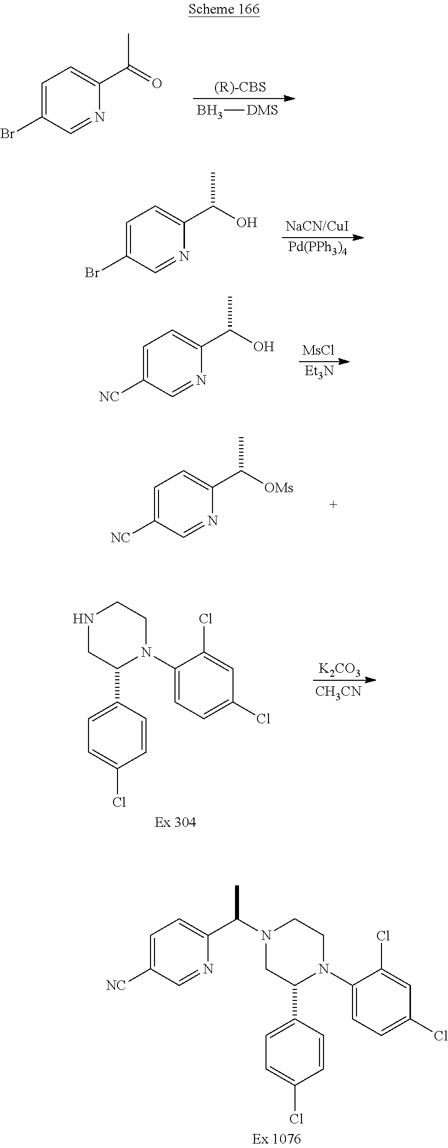

### Step 1

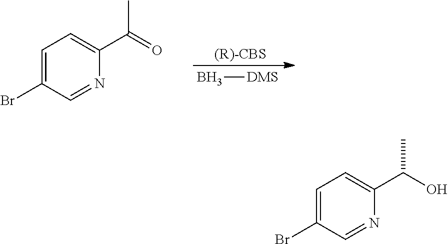

The ketone (1.0 g, 5 mmol) and (R)—CBS catalyst (1 ml of a 1.0 M solution in toluene) were taken up in THF (20 ml) at 0° C. Borane-dimethylsulfide complex (1.5 ml of a 2.0 M solution) was added dropwise to the reaction mixture at 0° C. The solution was stirred at 0° C. for 5 h. The reaction was allowed to warm to room temperature overnight (18 h). The reaction was quenched with 1 M HCl. The mixture was stirred at room temperature for 1 h. The solution was cooled to 0° C., and NaOH pellets were added to bring the pH up to 10-12. The mixture was extracted with EtOAc. The combined organic layers were washed with brine and dried (MgSO₄). Filtration and concentration gave a yellow oil. Purification via gradient flash chromatography (0-25% EtOAc in hexanes) gave 433 mg (43%) of the alcohol as a colorless oil.

### Step 2

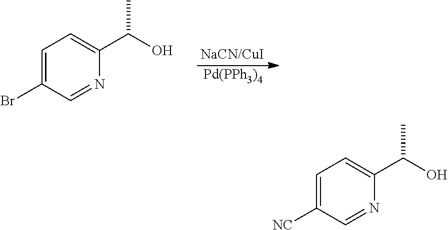

The alcohol (433 mg, 2.1 mmol), NaCN (210 mg, 4.3 mmol), Pd(PPh₃)₄ (247 mg, 0.21 mmol), and CuI (81 mg, 0.42 mmol) were taken up in EtCN (15 ml), and the resulting mixture was heated at 110° C. for 4 h. The solution was cooled and partitioned between EtOAc and water. The aqueous layer was extracted with EtOAc. The combined organic layers were washed with brine and dried (MgSO₄). Filtration and concentration gave a brown oil. Purification via gradient flash chromatography (0-50 EtOAc in hexanes) provided 35 mg (11%) of the cyano-pyridine as a yellow oil.

### Step 3

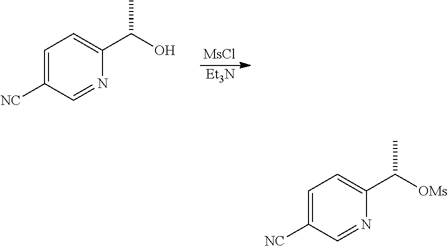

The alcohol (35 mg, 0.24 mmol) and Et3N (32 mg) were taken up in DCM (10 mL) and cooled to 0° C. Methanesulfonyl chloride (30 mg) was added, and the resulting solution was stirred at 0° C. for 1 h. The solution was diluted with DCM and washed with 1 M NaOH (aq.). The aqueous layer was extracted with DCM. The combined organic layers were dried (MgSO₄), filtered, and concentrated to afford the mesylate. The mesylate was used without further purification.

### Step 4

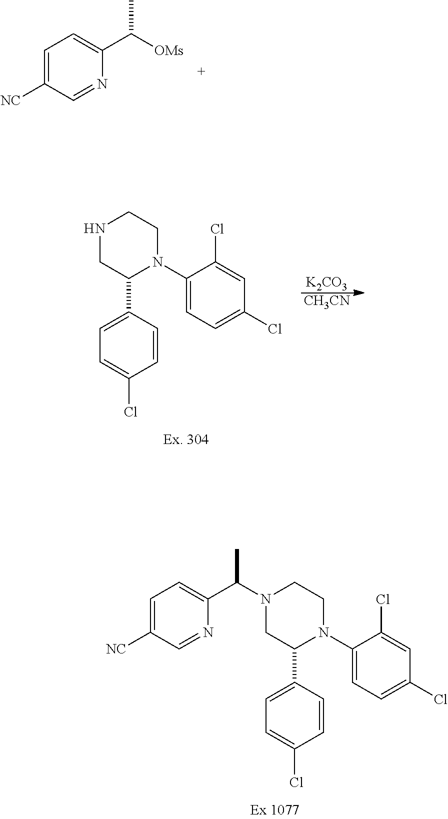

The mesylate, K₂CO₃ (83 mg), and piperazine (98 mg) were taken up in MeCN (15 ml), and the mixture was heated at 70° C. for 18 h. The solution was partitioned between EtOAc and water. The aqueous layer was extracted with EtOAc. The combined organic layers were washed with brine, dried (MgSO₄), filtered and concentrated to afford a yellow oil. Purification via preparative thin-layer chromatography (1/1 hexanes/EtOAc) gave a yellow foam. Further purification via Chiral HPLC (95/5 hexanes/IPA, semi-prep chiralpak AD column) which afforded 23 mg (20%) of Ex. 1077 as a colorless foam.

### Preparation of Example 1079

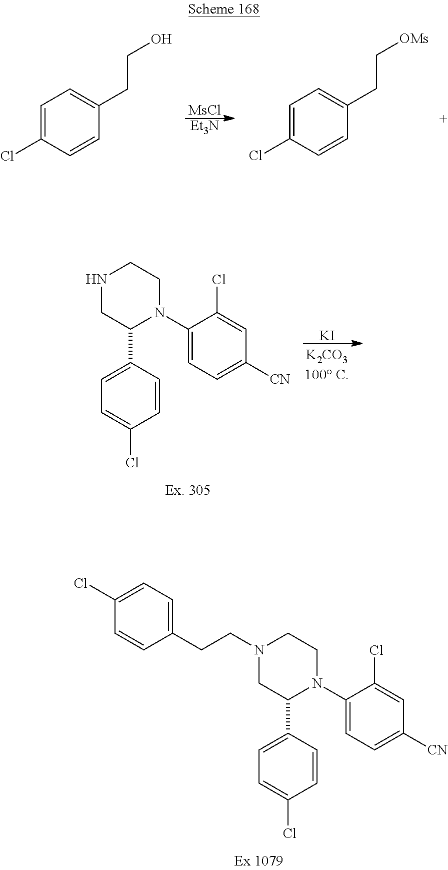

### Step 1

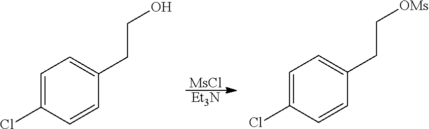 [SMILES: "Et3N" ; "MsCl" ; OCCc1ccc(Cl)cc1 ; CS(=O)(=O)OCCc1ccc(Cl)cc1]

The alcohol (500 mg, 3.2 mmol) and Et₃N (483 mg) were taken up in DCM (25 ml). Methanesulfonyl chloride (440 mg) was added to the solution, and the resulting solution was stirred at room temperature for 2 h. The solution was diluted with DCM and washed with sat. NaHCO_{3(aq.)}. The aqueous layer was extracted with DCM. The combined organic layers were dried (MgSO₄), filtered, and concentrated to give the mesylate as a yellow oil. The mesylate was used without further purification.

### Step 2

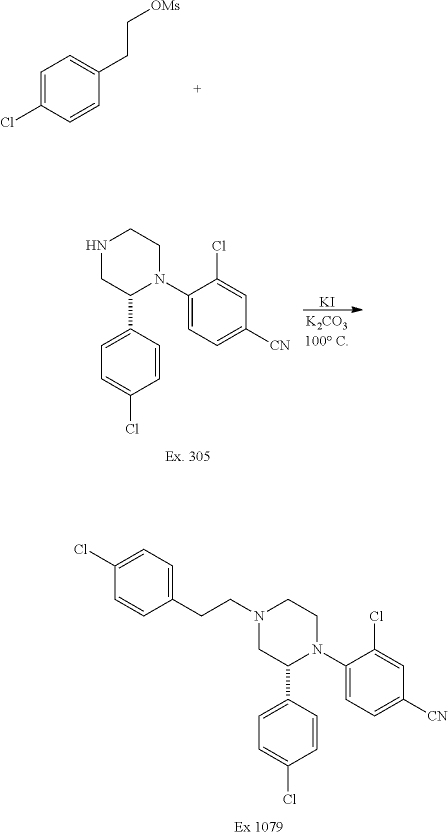 [SMILES: CS(=O)(=O)OCCc1ccc(Cl)cc1 ; N#Cc1ccc(N2CCNC[C@H]2c2ccc(Cl)cc2)c(Cl)c1 ; "KI" ; N#Cc1ccc(N2CCN(CCc3ccc(Cl)cc3)C[C@H]2c2ccc(Cl)cc2)c(Cl)c1]

The mesylate (70 mg), Ex. 305 (80 mg), Na (5 mg), and K₂CO₃ (80 mg) were taken up in CH₃CN and heated to 100° C. (18 h). The solution was partitioned between EtOAc and water. The aqueous layer was extracted with EtOAc. The combined organic layers were washed with brine and dried (MgSO₄). Filtration and concentration gave a yellow oil. Purification via thin-layer preparative chromatography (1/1 hexanes/EtOAc, SiO₂) gave 84 mg (74%) of Ex. 1079 as a colorless oil.

The following examples were prepared in a manner similar to that shown in Scheme 168 using the appropriate piperazine depicted in Table XLI.

TABLE XLI

| Piperazine in Step 2 | Ex | Structure |
|---|---|---|
| 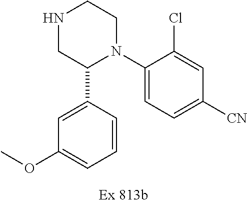 [SMILES: COc1cccc([C@@H]2CNCCN2c2ccc(C#N)cc2Cl)c1] | 1080 | 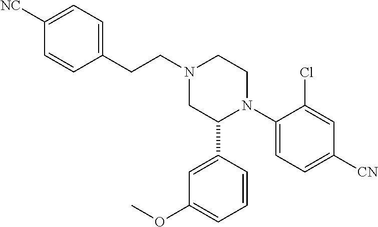 |
| 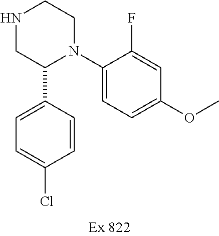 [SMILES: COc1ccc(N2CCNC[C@H]2c2ccc(Cl)cc2)c(F)c1] | 1081 | 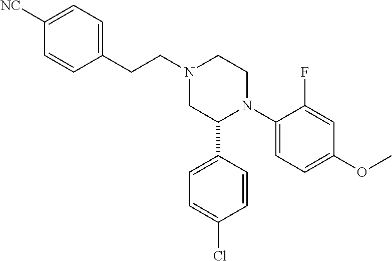 |
| 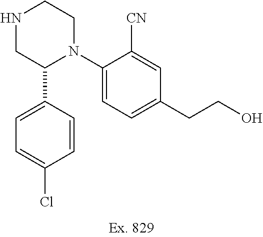 [SMILES: N#Cc1cc(CCO)ccc1N1CCNC[C@H]1c1ccc(Cl)cc1] | 1082 | 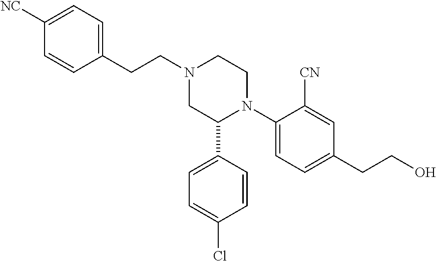 |
| 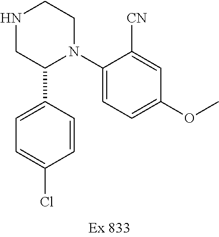 [SMILES: COc1ccc(N2CCNC[C@H]2c2ccc(Cl)cc2)c(C#N)c1] | 1082b | 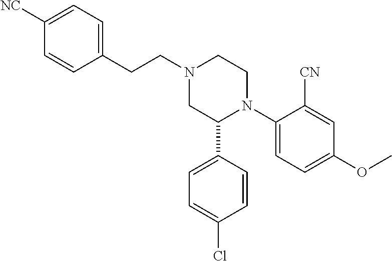 |
| 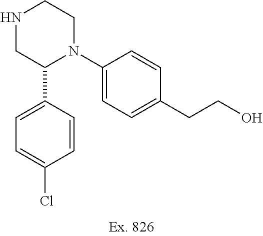 [SMILES: OCCc1ccc(N2CCNC[C@H]2c2ccc(Cl)cc2)cc1] | 1082c | 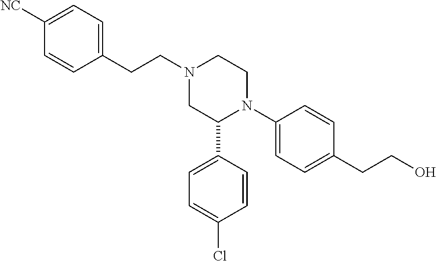 |

### Preparation of Example 1083

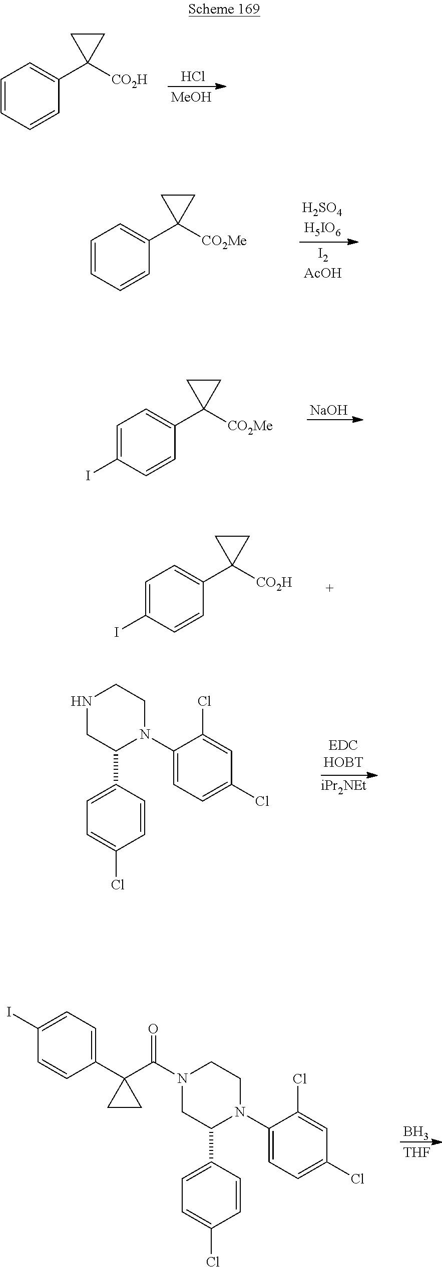

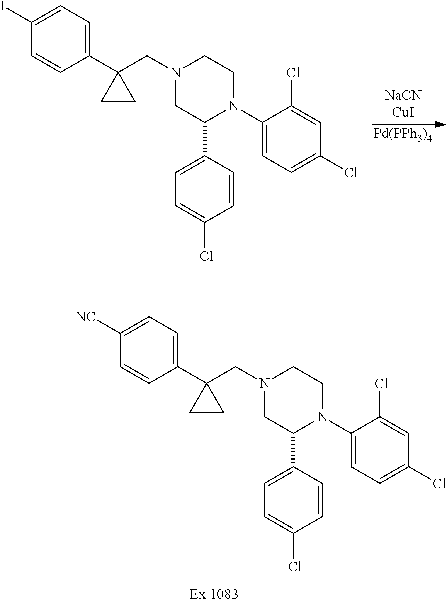

### Step 1

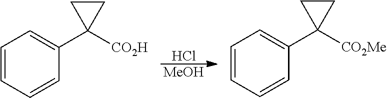 [SMILES: "MeOH" ; O=C(O)C1(c2ccccc2)CC1 ; Cl ; COC(=O)C1(c2ccccc2)CC1]

The acid (7.4 g, 45.6 mmol) was taken up in MeOH (35 ml) and 4.0 M HCl in dioxane (8 ml). The solution was heated at reflux for 18 h. The solution was concentrated. The residue was partitioned between Et₂O and H₂O. The aqueous layer was extracted with Et₂O. The combined organic layers were washed with sat. NaHCO_{3(aq.)}. The organic layers were dried (MgSO₄), filtered, and concentrated which furnished 7.9 grams (Quant.) of the methyl ester as a colorless oil.

### Step 2

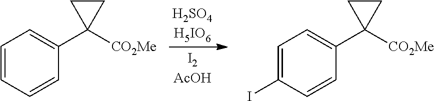 [SMILES: COC(=O)C1(c2ccccc2)CC1 ; COC(=O)C1(c2ccc(I)cc2)CC1]

The methyl ester (4.0 g, 23.3 mmol), H₂SO₄ (1.3 ml), H₅IO₆ (1.3 g), and iodine (2.7 g) were taken up in AcOH (30 ml) and heated at 60° C. (18 h). The solution was partitioned between EtOAc and 10% NaHSO₃(aq.). The aqueous layer was extracted with EtOAc. The combined organic layers were washed with brine. The organic layer was dried (MgSO₄), filtered, and concentrated which gave a semisolid. The residue was triturated with hexanes. The mixture was filtered, and the mother liquor was concentrated. The residue was purified via gradient flash chromatography (0-5% EtOAc in hexanes) which gave 1.64 g (24%) of the iodide as a colorless oil.

### Step 3

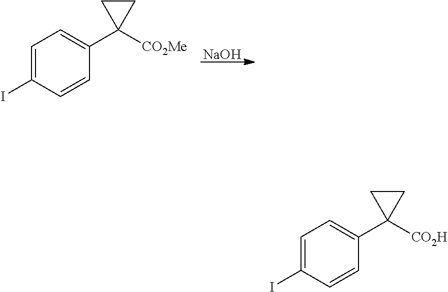

The methyl ester (1.64 g, 5.43 mmol) was taken up in 1 N NaOH_{(aq.)} and MeOH. The solution was heated at 60° C. for 18 h. The solution was concentrated. The residue was acidified with conc. HCl at 0° C. The mixture was extracted with DCM. The combined organic layers were dried (MgSO₄), filtered, and concentrated to afford a white solid. The residue was recrystallized from acetone/hexanes.

### Step 4

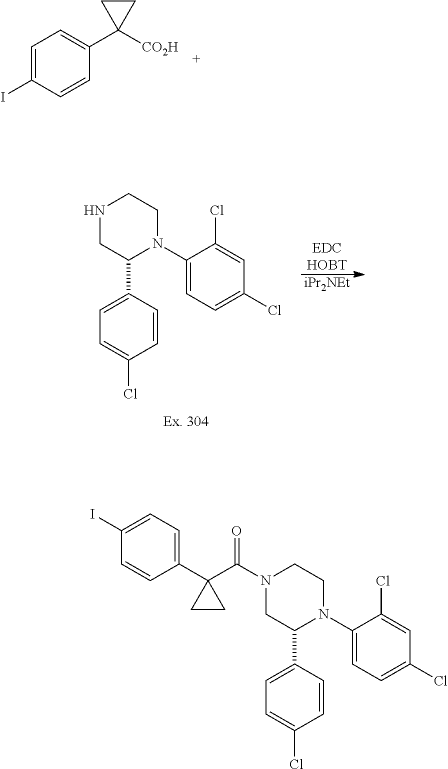 [SMILES: "iPr2NEt" ; O=C(O)C1(c2ccc(I)cc2)CC1 ; Clc1ccc([C@@H]2CNCCN2c2ccc(Cl)cc2Cl)cc1 ; "EDCHOBT" ; O=C(N1CCN(c2ccc(Cl)cc2Cl)[C@H](c2ccc(Cl)cc2)C1)C1(c2ccc(I)cc2)CC1]

The acid (152 mg, 0.53 mmol), Ex. 304 (150 mg, 0.44 mmol), EDC (110 mg, 0.57 mmol), HOBT (77 mg, 0.57 mmol), and iPr₂NEt (0.23 ml) were taken up in CH₃CN and heated at 65° C. for 18 h. The solution was cooled and concentrated. The residue was purified via thin-layer preparative chromatography (3/1 hexanes/EtOAc, SiO₂) to provide the amide as a colorless oil.

### Step 5

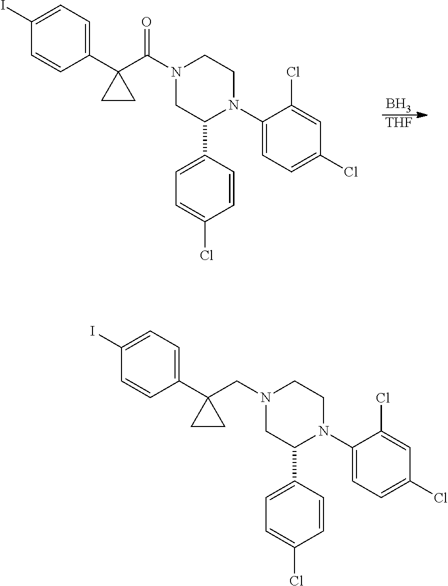

The amide was taken up THF (15 ml) and BH₃-THF (1.0 M in THF, 3 ml) was added. The solution was stirred at 65° C. for 18 h. The solution was quenched with MeOH (0.5 ml) followed by 1 M HCl_{(aq.)} (15 ml). The solution was heated at 65° C. for 2 h. The solution was cooled and diluted with water. The mixture was made basic via addition of NaOH pellets. The mixture was extracted with EtOAc. The combined organic layers were washed with brine and dried (MgSO₄). Filtration and concentration gave a brown oil. Purification via thin-layer preparative chromatography (9/1 hexanes/EtOAc, SiO₂) gave 54 mg (21% from the piperazine in Step 4) of the piperazine as a colorless oil.

### Step 6

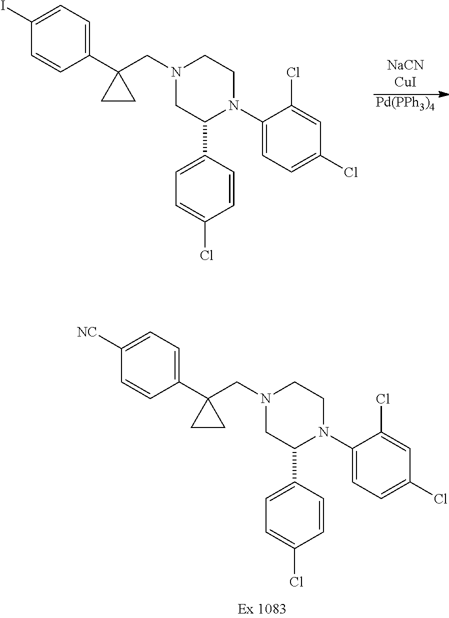 [SMILES: Clc1ccc([C@@H]2CN(CC3(c4ccc(I)cc4)CC3)CCN2c2ccc(Cl)cc2Cl)cc1 ; N#Cc1ccc(C2(CN3CCN(c4ccc(Cl)cc4Cl)[C@H](c4ccc(Cl)cc4)C3)CC2)cc1 ; "Pd(PPh3)4"]

The iodide (54 mg, 0.09 mmol), NaCN (10 mg), Pd(PPh₃)₄ (10 mg), and CuI (3 mg) were taken up in EtCN and heated at 115° C. for 4 h. The solution was cooled and partitioned between EtOAc and sat. NaHCO_{3(aq.)}. The aqueous layer was extracted with EtOAc. The combined organic layers were washed with brine and dried (MgSO₄). Filtration and concentration gave a brown oil. Purification via thin-layer preparative chromatography (6/1 hexanes/EtOAc, SiO₂) provided 16 mg (34%) of Ex. 1083 as a colorless oil.

### Preparation of Example 1084

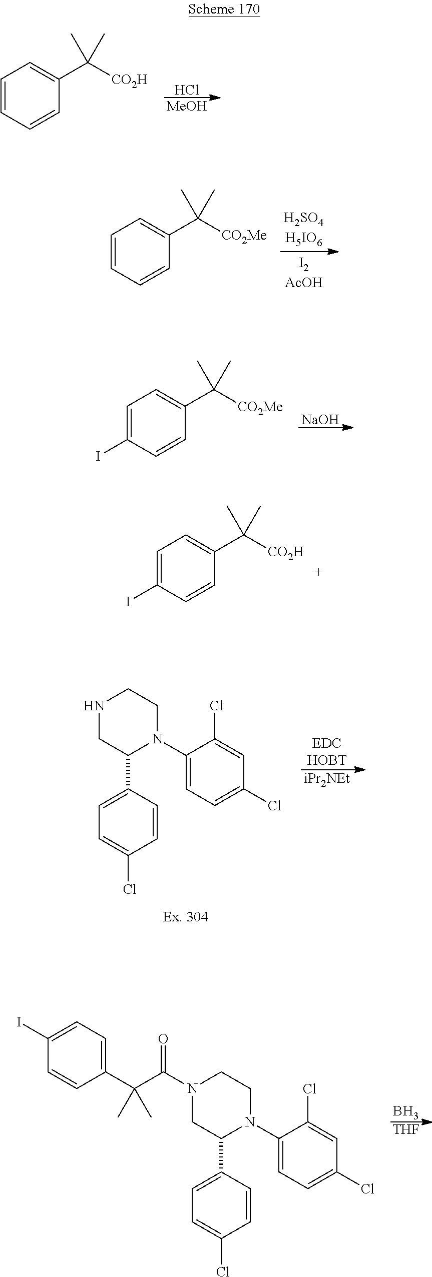

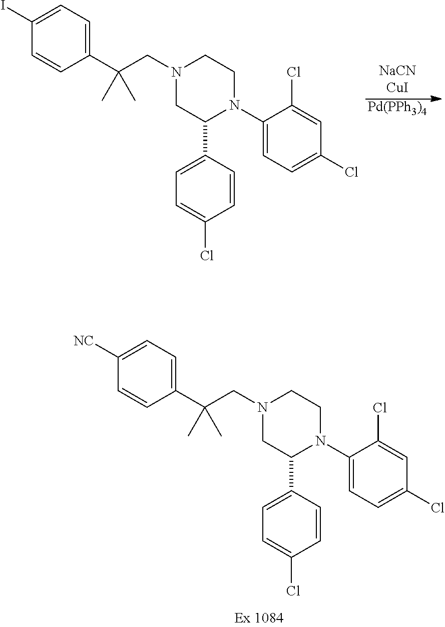 [SMILES: CC(C)(CN1CCN(c2ccc(Cl)cc2Cl)[C@H](c2ccc(Cl)cc2)C1)c1ccc(I)cc1 ; CC(C)(CN1CCN(c2ccc(Cl)cc2Cl)[C@H](c2ccc(Cl)cc2)C1)c1ccc(C#N)cc1 ; "Pd(PPh3)4"]

The dimethyl carboxylic acid was processed as described for the cyclopropyl acid in Scheme 169 to provide Ex. 1084 (Scheme 170).

### Preparation of Example 1085

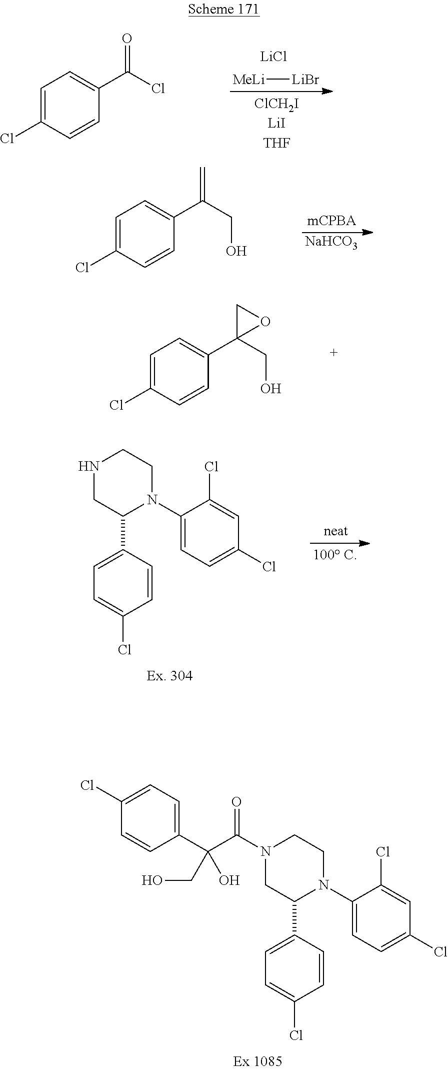

### Step 1

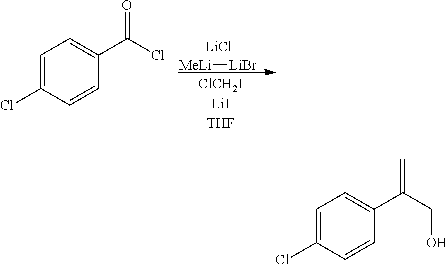

The acid chloride (3.5 g, 20 mmol), LiBr (3.78 g, 44 mmol), and ClCH₂I (3.2 ml) were taken up in THF (45 ml) and cooled to −78° C. Methyl lithium LiBr in Et₂O) (30 ml of a 1.5 M solution) was added dropwise to the solution at −78° C. The resulting solution was stirred at −78° C. for 1 h. The solution was warmed to 25° C. and stirred at that temperature for 18 h. The solution was quenched with sat. NH₄Cl_{(aq.)} and concentrated. The residue was partitioned between EtOAc and 10% Na₂S₂O_{3(aq.)}. The aqueous layer was extracted with EtOAc. The combined organic layers were washed with brine, dried (MgSO₄), filtered, and concentrated. The residue was purified via gradient flash chromatography (hexanes to 20% EtOAc in hexanes, SiO₂) provided 520 mg (15%) of the allylic alcohol as a colorless oil.

### Step 2

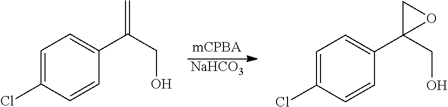

The allylic alcohol (520 mg, 3.1 mmol), mCPBA (834 mg, 3.7 mmol), and NaHCO₃ (521 mg, 6.2 mmol) were partitioned between DCM and water. The mixture was stirred at 25° C. for 18 h. The layers were separated, and the organic layer was washed with 10% Na₂S₂O_{3(aq.)}. The organic layer was dried (MgSO₄), filtered, and concentrated to give a yellow oil. The residue was purified via thin-layer preparative chromatography (2/1 hexanes/EtOAc, SiO₂) to furnish 336 mg (59%) of the epoxide as a colorless oil.

### Step 3

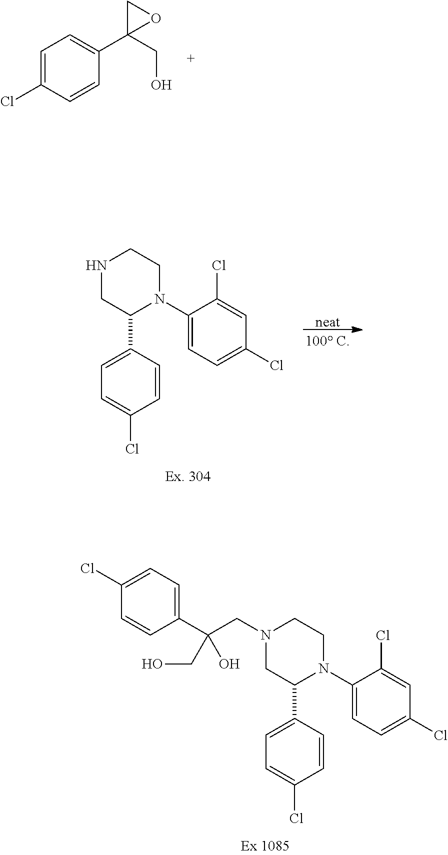 [SMILES: OCC1(c2ccc(Cl)cc2)CO1 ; Clc1ccc([C@@H]2CNCCN2c2ccc(Cl)cc2Cl)cc1 ; "neat" ; OCC(O)(CN1CCN(c2ccc(Cl)cc2Cl)[C@H](c2ccc(Cl)cc2)C1)c1ccc(Cl)cc1]

The epoxide (336 mg, 1.82 mmol) and Ex. 304 (250 mg, 1.2 mmol) were heated neat (100° C.) for 18 h. The residue was purified via gradient flash chromatography (0-30% EtOAc in hexanes, SiO₂) gave a yellow oil. The residue was furthered purified via thin-layer preparative chromatography (4/1 DCM/EtOAc, SiO₂) to furnish 31 mg (5%) of Ex. 1085 as a colorless oil.

### Preparation of Example 1086

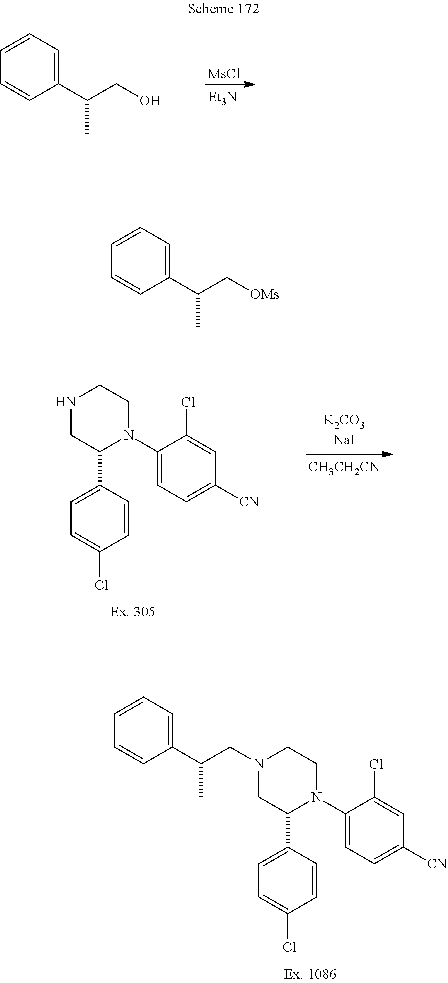

### Step 1

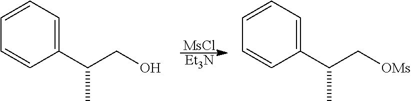

The alcohol (500 mg, 3.68 mmol) and Et₃N (0.6 ml) were taken up in DCM (25 ml) and cooled to 0° C. Methanesulfonyl chloride (0.3 ml) was added dropwise at 0° C. The solution was warmed to 25° C. and stirred at that temperature for 2 h. The solution was diluted with DCM and washed with sat. NaHCO_{3(aq.)}. The aqueous layer was extracted with DCM. The combined organic layers were dried (MgSO₄), filtered and concentrated to furnish 790 mg (Quant) of the mesylate as a yellow oil.

### Step 2

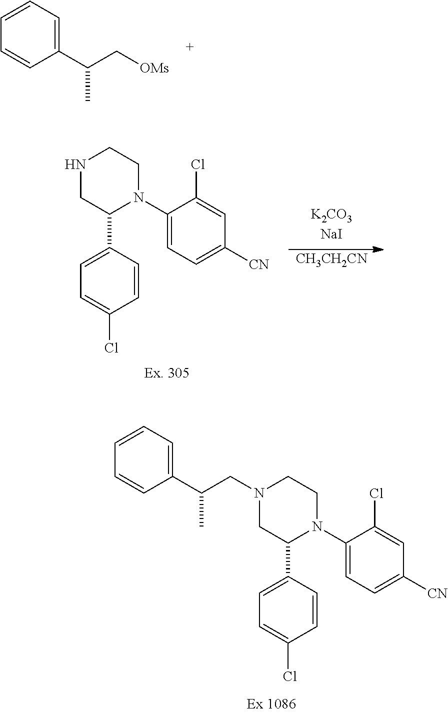

The mesylate (103 mg, 0.48 mmol), piperazine (80 mg, 0.24 mmol), K₂CO₃ (200 mg), and NaI (36 mg) were taken up in CH₃CH₂CN (15 ml), and the mixture was heated at 100° C. for 18 h. The solution was partitioned between EtOAc and 1 N NaOH_{(aq.)}. The aqueous layer was extracted with EtOAc. The combined organic layers were washed with brine and dried (MgSO₄). The mixture was filtered and concentrated. The residue was purified via thin-layer preparative chromatography (3/1 hexanes/EtOAc, SiO₂) to provide 38 mg (35%) of the Ex. 1086 as a white solid.

### Preparation of Example 1087a

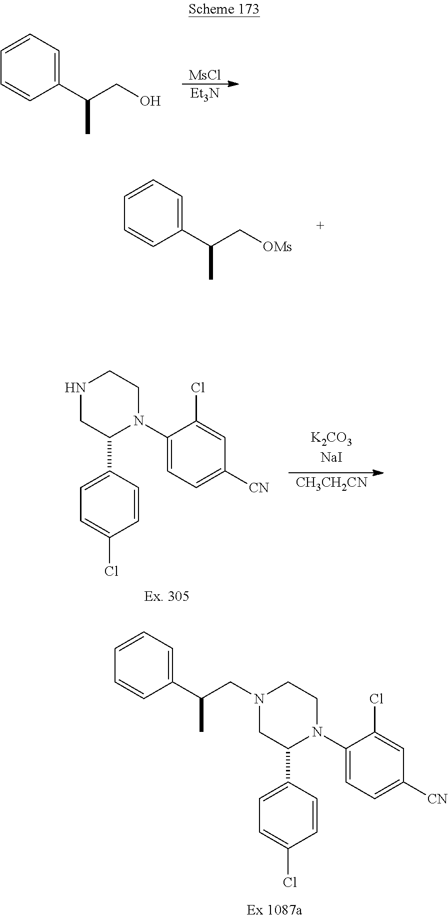

The mesylate was processed as described in Scheme 172 to provide Ex 1087a as a white solid.

### Preparation of Example 1087b

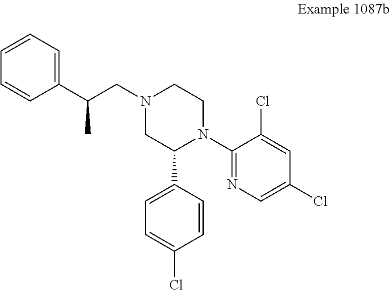 [SMILES: C[C@H](CN1CCN(c2ncc(Cl)cc2Cl)[C@H](c2ccc(Cl)cc2)C1)c1ccccc1]

Example 1087b was prepared in the same fashion as Example 1087a except that piperazine Example 826c was used instead of Example 305 in step 2.

### Preparation of Example 1088

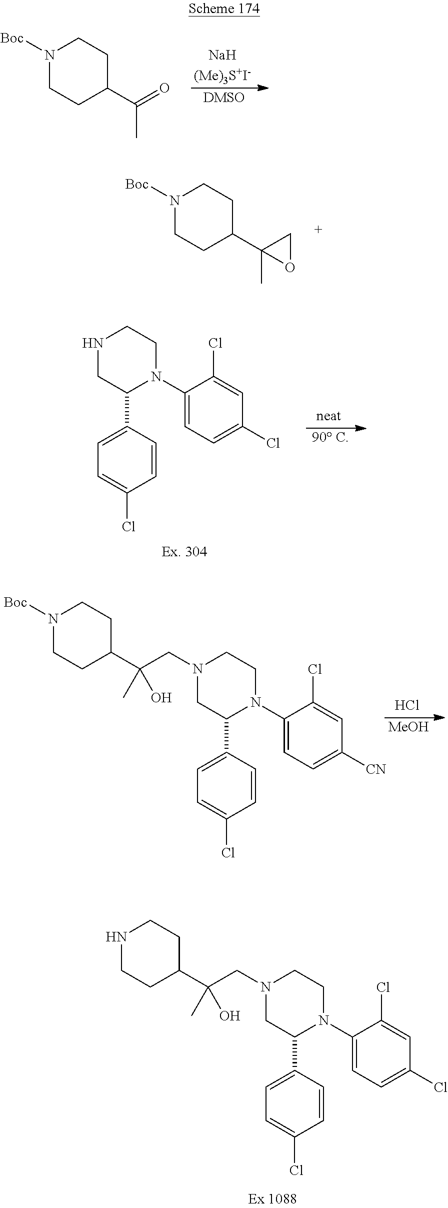

### Step 1

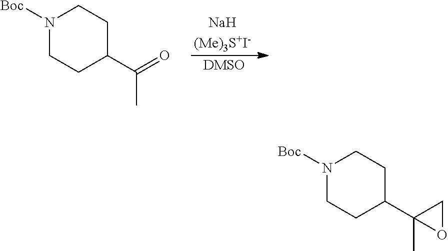

The (Me)_{3S} ⁺I⁻ (5.3 g, 26 mmol) was taken up in DMSO (15 ml). Sodium hydride (1.1 g, 60 wt % dispersion in oil) was added to the solution (gas evolution). The resulting solution was stirred at 25° C. for 45 minutes. The ketone (5.0 g in 10 ml of DMSO) was added to the solution. The resulting solution was stirred at 25° C. for 18 h. The solution was quenched with water, and the mixture was extracted with Et₂O. The combined Et₂O layers were washed with water and brine. The combined organic layers were dried (MgSO₄), filtered, and concentrated to provide the epoxide as a yellow oil The epoxide was used without further purification.

### Step 2

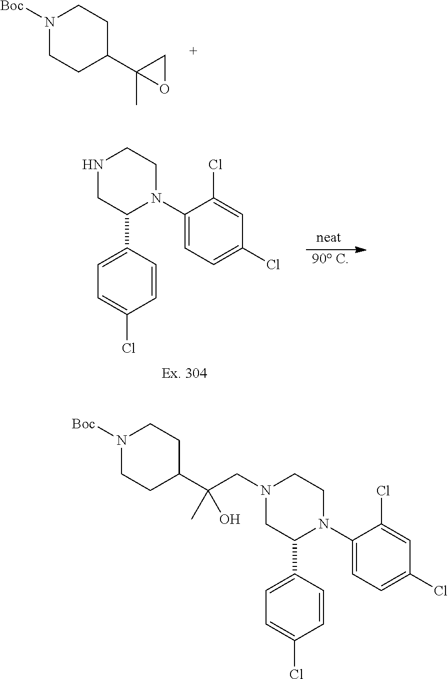

The epoxide (370 mg, 1.54 mmol) and Ex. 304 (350 mg, 1.02 mmol) were heated neat in a sealed tube at 90° C. for 18 h. More epoxide (0.4 ml) and toluene (3 ml) was added, and the resulting solution was heated at 100° C. for 18 h. The mixture was concentrated. The residue was purified via gradient flash chromatography (0-30% EtOAc in hexanes, SiO₂) which provided 262 mg (29%) of the Boc protected piperidine as a colorless foam.

### Step 3

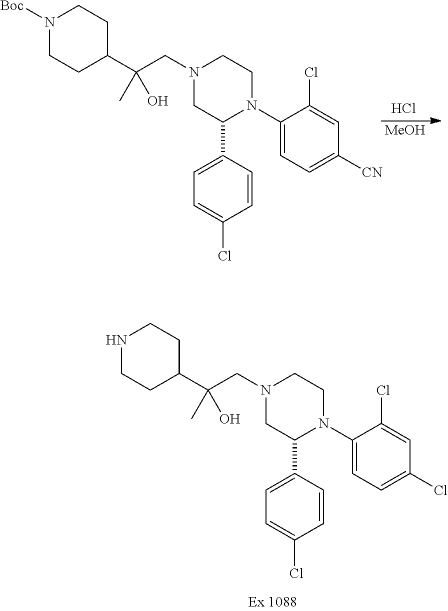

The Boc protected piperidine (260 mg, 0.45 mmol) and 4.0 M HCl in dioxane (2 ml) were taken up in MeOH (15 ml). The solution was stirred at 25° C. for 18 h. The solution was concentrated. The residue was partitioned between DCM and 1 N NaOH_{(aq.)}. The aqueous layer was extracted with DCM. The combined organic layers were dried (MgSO₄), filtered, and concentrated to provide 217 mg (Quant.) of Ex. 1088 as a colorless foam

### Preparation of Example 1089

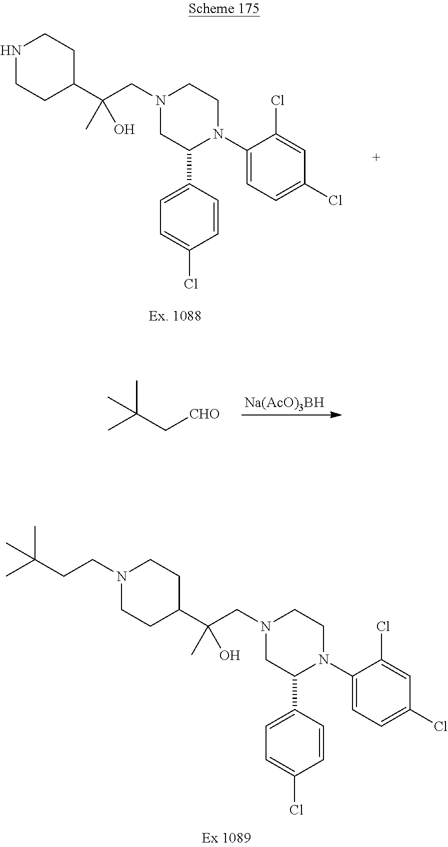

Ex. 1088 (50 mg, 0.1 mmol), the aldehyde (0.1 ml), and sodium triacetoxy borohydride (64 mg) were taken up in DCM and stirred at 25° C. for 48 h. The mixture was diluted with DCM and washed with 1 N NaOH_{(aq.)}. The aqueous layer was extracted with DCM. The combined organic layers were dried (MgSO₄), filtered, and concentrated. The residue was purified via thin-layer preparative chromatography (½ hexanes/acetone, SiO₂) to provide 34 mg (60%) of Ex. 1089 as a colorless oil.

### Preparation of Example 1090

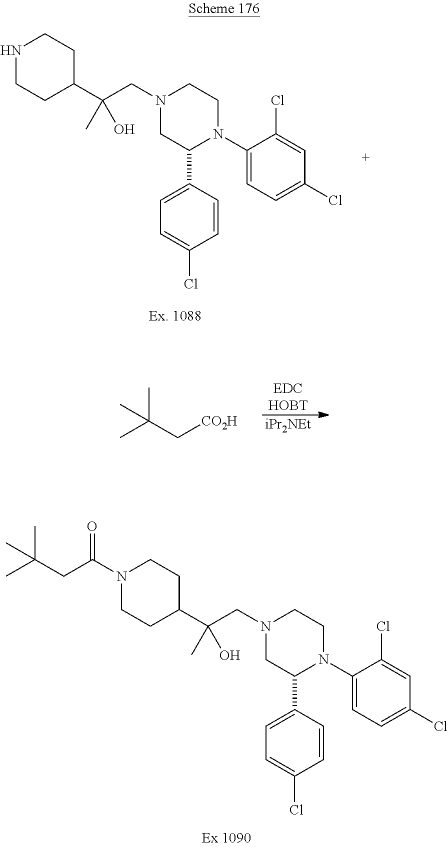

Ex. 1088 (50 mg, 0.1 mmol), EDC (24 mg), HOBT (17 mg), acid (23 mg), and iPr₂NEt (0.1 ml) were taken up in DCM and stirred at 25° C. for 18 h. The solution was diluted with DCM and washed with 1 N NaOH_{(aq.)}. The aqueous layer was extracted with DCM. The combined organic layers were dried (MgSO₄), filtered, and concentrated. The residue was purified via thin-layer preparative chromatography (4/1 hexanes/acetone, SiO₂) to provide 57 mg (98%) of the Ex. 1090 as a colorless oil.

### Preparation of Example 1091

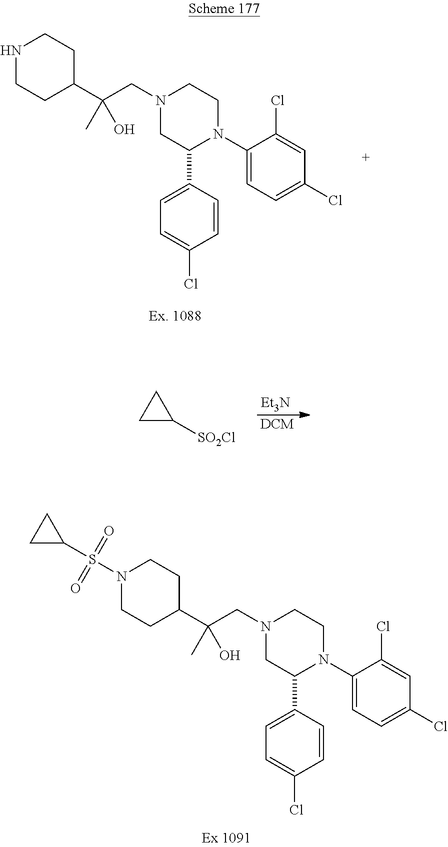

Ex. 1088 (50 mg, 0.1 mmol) and triethylamine (0.1 ml) were taken up in DCM. Cylcopropyl sulfonyl chloride (30 mg) was added, and the solution was stirred at 25° C. for 2.5 h. The solution was concentrated. The residue was purified via thin-layer preparative chromatography (4/1 hexanes/acetone, SiO₂) which provided 43 mg (73%) of Ex. 1091 as a colorless oil.

### Preparation of Example 1092

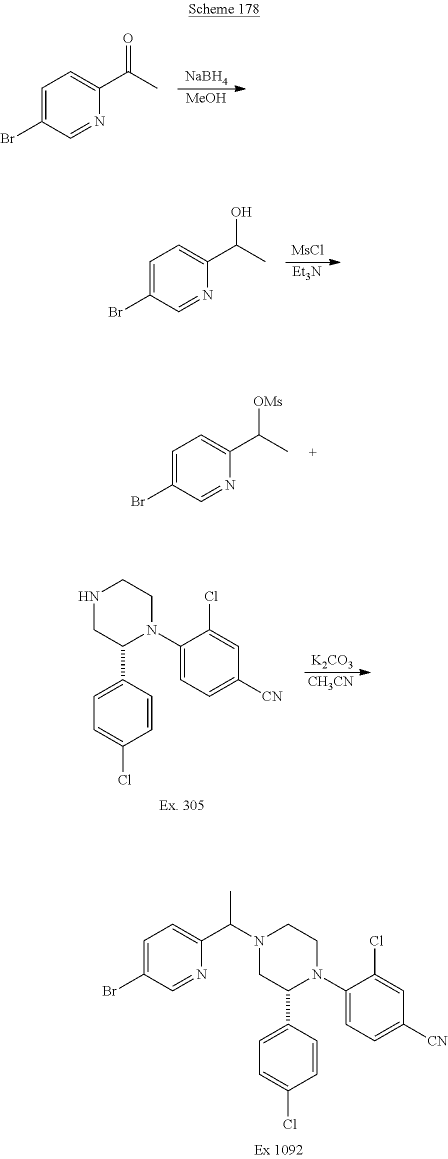

### Step 1

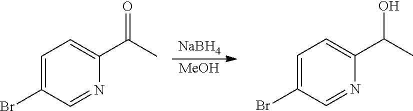

The ketone (1.0 g, 5 mmol) was taken up in MeOH (25 ml). Sodium borohydride (230 mg, 6 mmol) was added (gas evolution). The solution was stirred at 25° C. for 4 h. The solution was concentrated. The residue was taken up in 1 M HCl_{(aq.)} and stirred at 25° C. for 0.5 h. The solution was made basic via addition of NaOH pellets. The mixture was extracted with DCM. The combined organic layers were dried (MgSO₄), filtered, and concentrated. This yielded 1.0 g (Quant.) of the alcohol as a yellow oil.

### Step 2

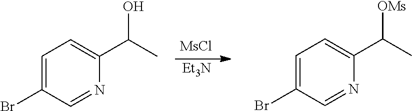

The alcohol (1.0 g, 5 mmol) and Et₃N (0.8 ml) were taken up in DCM (35 ml), and the solution was cooled to 0° C. Methanesulfonyl chloride (0.43 ml) was added, and the resulting solution was stirred at 0° C. for 2 h. The solution was diluted with DCM and washed with 1 N NaOH_{(aq.)}. The aqueous layer was extracted with DCM. The combined organic layers were dried (MgSO₄), filtered, and concentrated to furnish 1.3 g (93%) of the mesylate as a yellow oil.

### Step 3

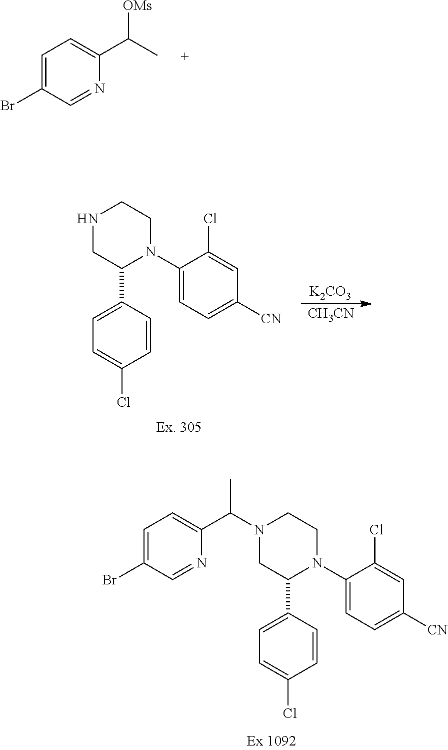

The mesylate (700 mg, 2.5 mmol), Ex. 305 (692 mg, 2.1 mmol), and K₂CO₃ (580 mg, 4.2 mmol) were taken up in CH₃CN (20 ml) and heated at 90° C. for 18 h. The solution was partitioned between EtOAc and 1 N NaOH_{(aq.)}. The aqueous layer was extracted with EtOAc. The combined organic layers were washed with brine and dried (MgSO₄). Filtration and concentration provided a yellow oil. Purification via gradient flash chromatography (0-35% EtOAc in hexanes, SiO₂) provided 912 mg (84%) of Ex. 1092.

### Preparation of Example 1093 and 1094

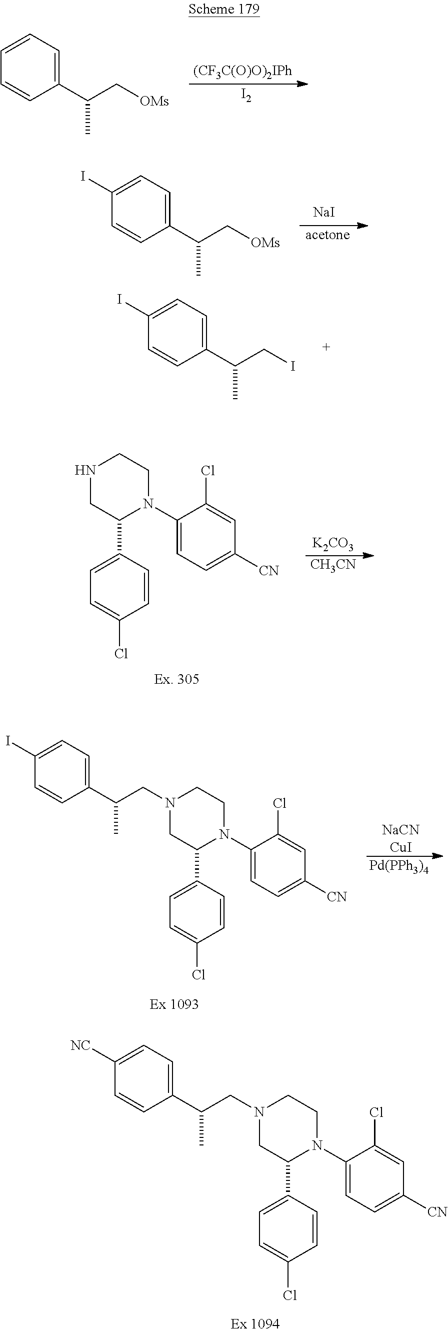

### Step 1

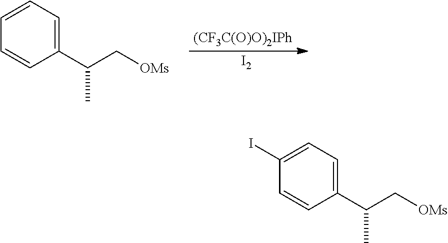

The mesylate from Step 1 of Scheme 172 (1.66 g, 7.76 mmol), iodine (985 mg, 3.88 mmol), and (CF₃C(O)O)₂IPh (2 g, 4.66 mmol) were taken up in DCM (65 ml) and stirred at 25° C. for 18 h. The mixture was poured into an aqueous solution containing NaHCO₃ (2 g) and NaHSO₃ (700 mg). The mixture was stirred at 25° C. for 0.5 h. The aqueous layer was extracted with DCM. The combined organic layers were dried (MgSO₄), filtered, and concentrated. The residue was purified via gradient flash chromatography (0-25% EtOAc in hexanes, SiO₂) which provided 1.73 g (66%) of the mesylate as a colorless oil.

### Step 2

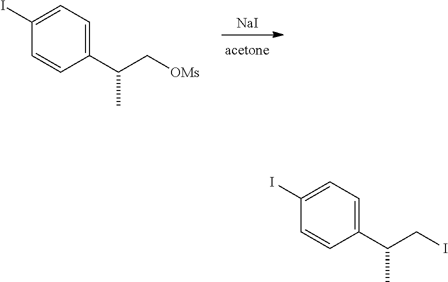

The mesylate (1.73 g, 5.09 mmol) and NaI (7.6 g, 50 mmol) were taken up in acetone (80 ml) and heated at 70° C. for 18 h. The mixture was partitioned between Et₂O and water. The aqueous layer was extracted with Et₂O. The combined organic layers were washed with 10% Na₂S₂O_{3(aq.)} and dried over MgSO₄. Filtration and concentration provided 1.86 g (98%) of the iodide as a yellow oil.

### Step 3

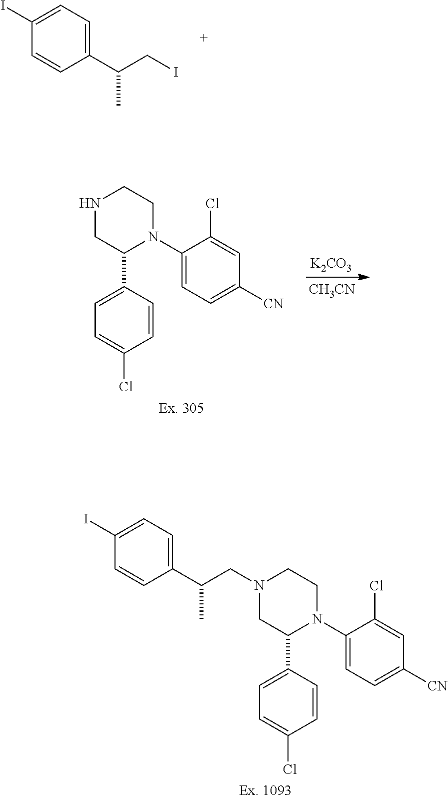

The iodide (446 mg, 1.2 mmol), Ex. 305 (200 mg, 0.6 mmol), and K₂CO₃ (207 mg, 1.5 mmol) were taken up in CH₂CH₂CN (4 ml), and the resulting mixture was heated at 100° C. in a sealed tube for 18 h. More iodide (0.1 ml) and K₂CO₃ (100 mg) were added, and the resulting mixture was stirred at 115° C. for 18 h. The mixture was partitioned between EtOAc and 1 N NaOH_{(aq.)}. The aqueous layer was extracted with EtOAc. The combined organic layers were washed with brine and dried (MgSO₄). Filtration and concentration gave a yellow oil. The residue was purified via gradient flash chromatography (0-20% EtOAc in hexanes, SiO₂) which provided 220 mg (64%) of Ex. 1093 as a yellow foam.

### Step 4

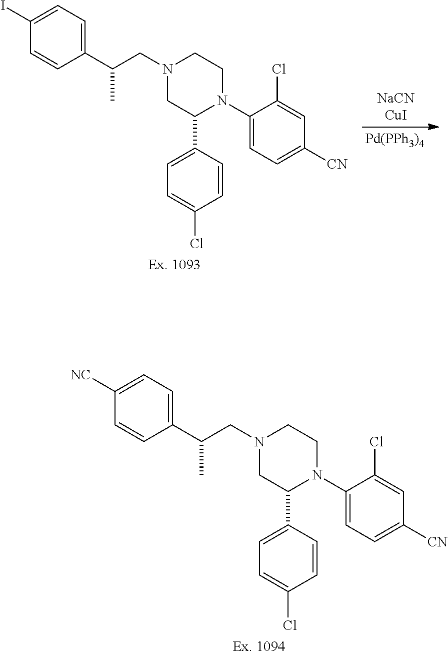

The iodide (Ex. 1093: 188 mg, 0.32 mmol), NaCN (32 mg, 0.65 mmol), CuI (12 mg, 0.065 mmol), and Pd(PPh₃)₄ (38 mg, 0.032 mmol) were taken up in degassed EtCN (10 ml), and the resulting mixture was heated at 105° C. for 1 h. The solution was partitioned between EtOAc and water. The aqueous layer was extracted with EtOAc. The combined organic layers were washed with brine and dried (MgSO₄). Filtration and concentration gave a yellow oil. The residue was purified via preparative thin-layer chromatography (2/1 hexanes/EtOAc, SiO₂) which gave 39 mg (25%) of Ex. 1094 as a colorless oil.

### Preparation of Example 1095 and 1096

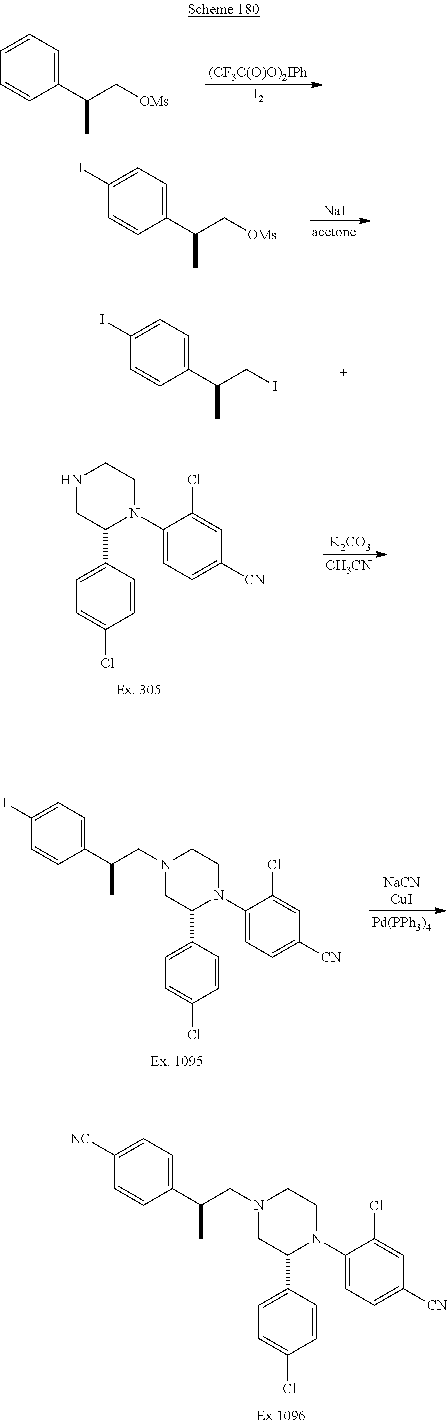

Example 1095 and 1096 were prepared as outlined in Scheme 179 using the mesylate shown in Scheme 180.

### Preparation of Example 1097

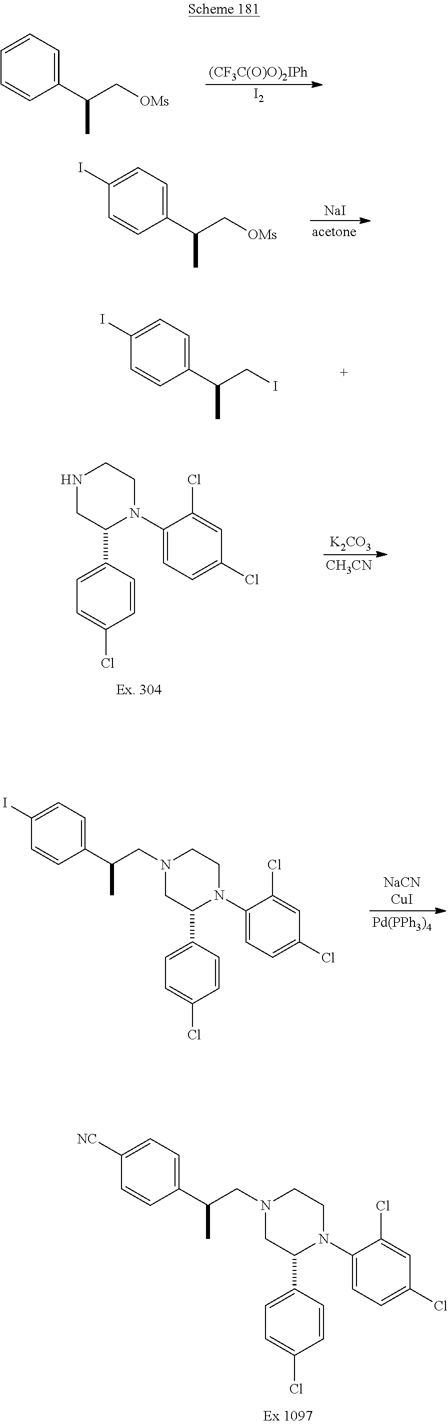

Example 1097 was prepared according the steps outlined in Scheme 179 using the Ex. 304(Scheme 181).

### Preparation of Example 1098

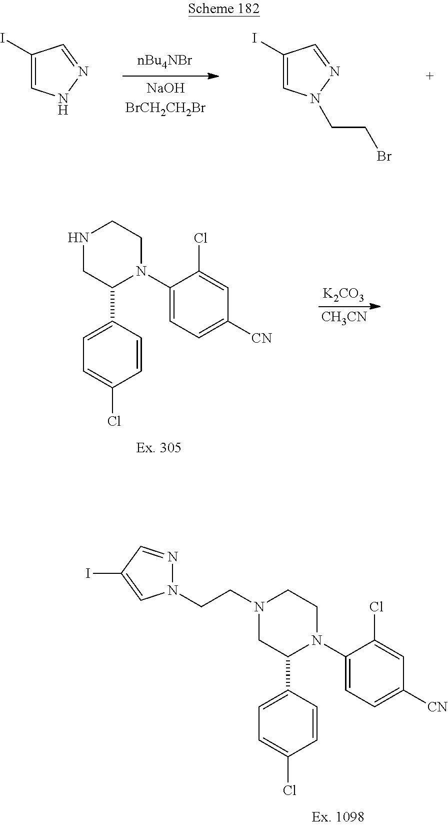

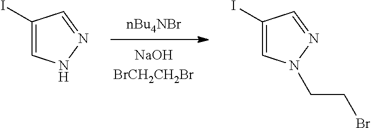 [SMILES: Ic1cn[nH]c1 ; BrCCn1cc(I)cn1]

The iodo-pyrazole (8 g, 41 mmol) and nBu₄NBr (1.3 g) were partitioned between BrCH₂CH₂Br (80 ml) and 12 ml of 40% NaOH_{(aq.)}. The mixture was stirred vigorously at 25° C. for 18 h. The mixture was partitioned between DCM and water. The aqueous layer was extracted with DCM. The combined organic layers were dried (MgSO₄). Filtration and concentration gave a yellow oil. The residue was filtered through a plug of SiO₂ (eluting with DCM). Concentration of the solution provided the bromo-pyrazole as a yellow oil.

### Step 2

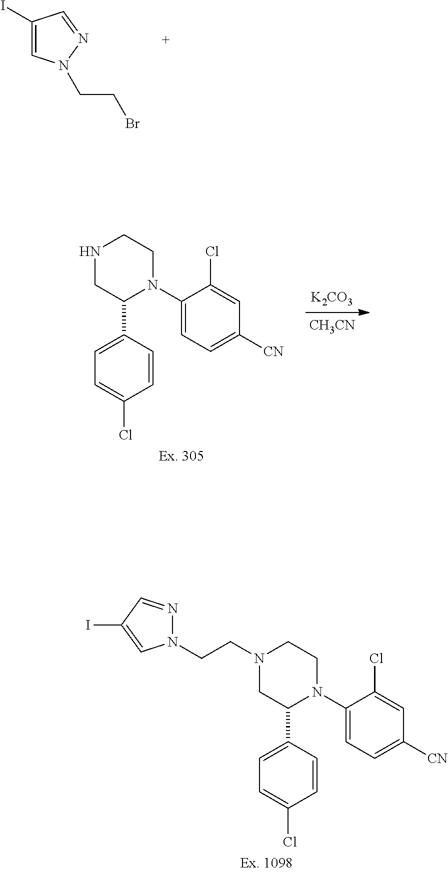

The iodo-pyrazole (210 mg), Ex. 305 (80 mg), and K₂CO₃ (66 mg) were taken up in EtCN (5 ml) and heated in a sealed tube at 115° C. for 18 h. The mixture was partitioned between EtOAc and water. The aqueous layer was extracted with EtOAc. The combined organic layers were washed with brine and dried (MgSO₄). Filtration and concentration gave a yellow oil. The residue was purified via thin-layer preparative chromatography (15% EtOAc in DCM, SiO₂) provided 100 mg (75%) of Ex. 1098 as a colorless oil.

### Preparation of Example 1099

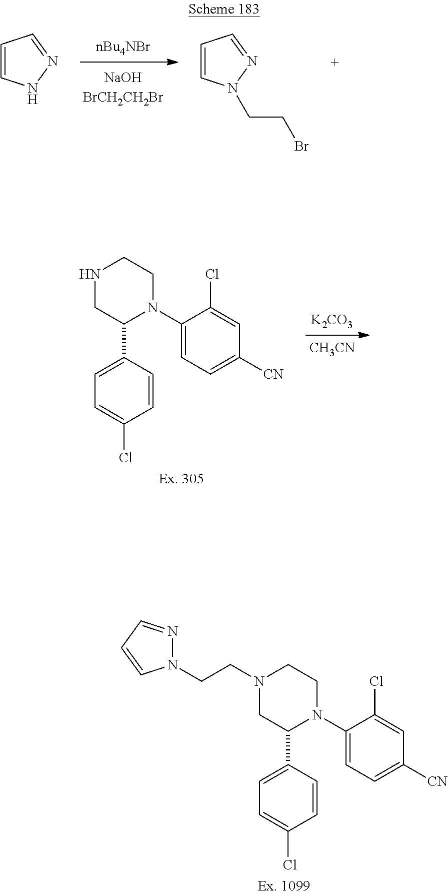

Example 1099 was prepared in a similar manner as depicted in Scheme 182 using the appropriate pyrazole (Scheme 183).

### Preparation of Example 1100 and 1101

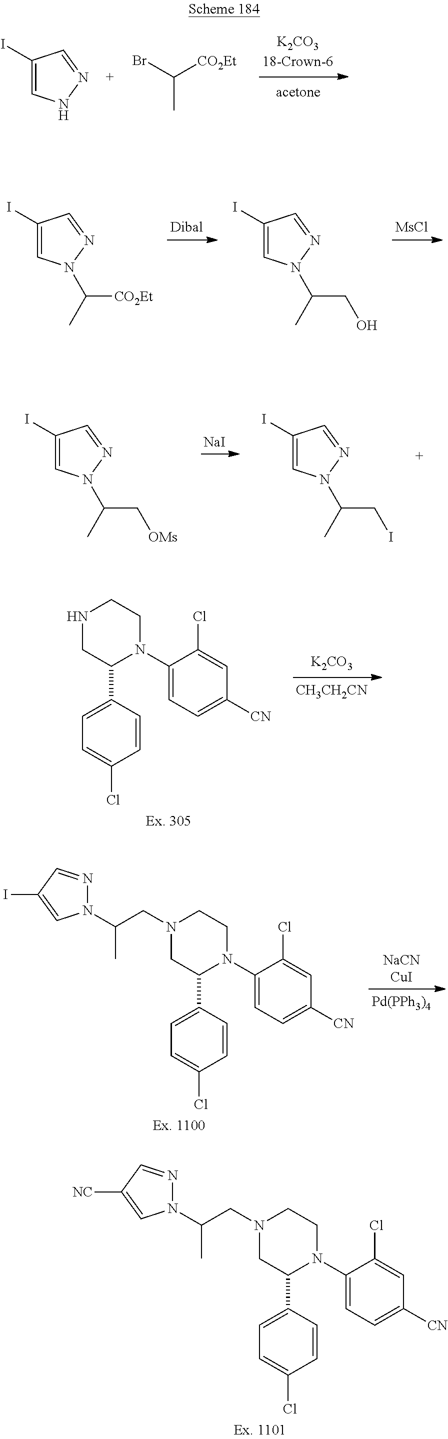

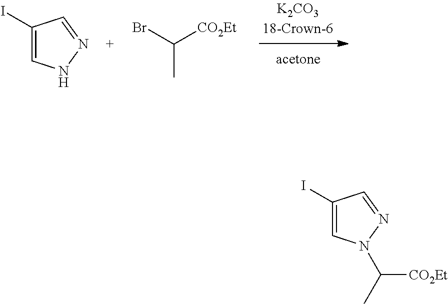

### Step 1

The iodo-pyrazole (8 g, 41.5 mmol), ethyl bromo-propionate (7.2 g, 42 mmol), K₂CO₃ (10 g), and 18-Crown-6 (140 mg) were taken up in acetone (80 ml) and heated at 60° C. for 20 h. The solution was cooled and concentrated. The residue was partitioned between Et₂O and water. The aqueous layer was extracted with Et₂O. The combined organic layers were washed with brine and dried (MgSO₄). Filtration and concentration gave a yellow oil. The residue was purified via gradient flash chromatography (0-50% EtOAc in DCM, SiO₂) which gave the pyrazole ester as a yellow oil.

### Step 2

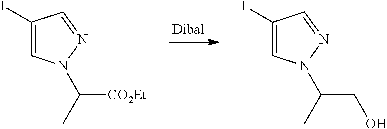

The pyrazole ester (1 g, 3.5 mmol) was taken up in toluene (10 ml) and cooled to 0° C. Diisobutylaluminum hydride (5.3 ml of a 1.5 M solution in toluene) was added to the solution at 0° C. The solution was warmed to 25° C. and stirred at that temperature for 20 h. The reaction was quenched with MeOH (5 ml), and the resulting solution was poured into a sat. NaK tartrate_{(aq.)} solution (50 ml). The mixture was stirred at 25° C. for 2 h. The layers were separated, and the aqueous layer was extracted with EtOAc. The combined organic layers were washed with brine and dried (MgSO₄). Filtration and concentration provided 800 mg (93%) of the alcohol as a yellow oil.

### Step 3

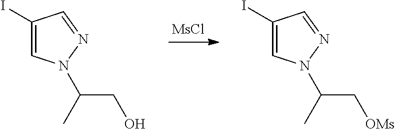

The alcohol (800 mg, 3.3 mmol) and Et₃N (440 mg) were taken up in DCM (15 ml). Methanesulfonyl chloride (430 mg) was added, and the resulting solution was stirred at 25° C. for 0.5 h. The solution was diluted with DCM and washed with sat. NaHCO_{3(aq)}. The aqueous layer was extracted with DCM. The combined organic layers were dried (MgSO₄). Filtration and concentration gave 800 mg (76%) of the mesylate as a yellow oil.

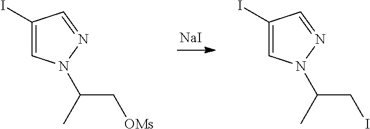

### Step 4

The mesylate (800 mg, 2.5 mmol) and NaI (3.74 g) were taken up in acetone (15 ml) and heated at 70° C. for 20 h. The solution was concentrated, and the residue was partitioned between Et₂O and 10% Na₂S₂O₃ (aq.). The aqueous layer was extracted with Et₂O. The combined organic layers were washed with brine and dried (MgSO₄). Filtration and concentration gave the iodide as a yellow oil.

### Step 5

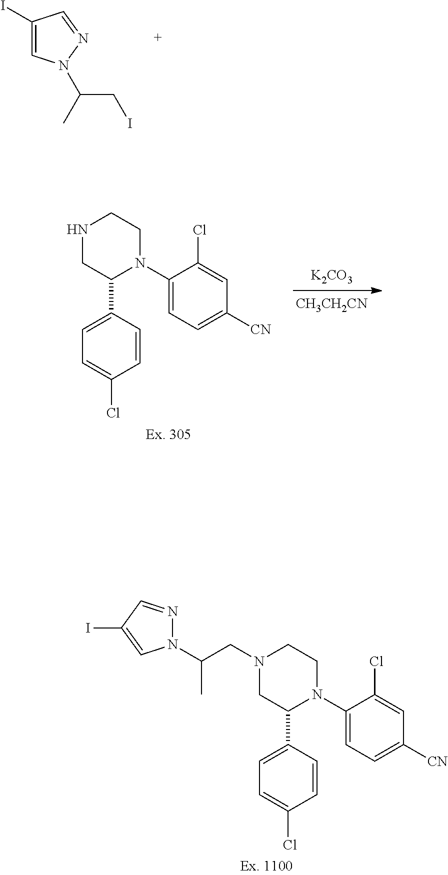

The iodide (250 mg), piperazine (80 mg, 0.24 mmol), and K₂CO₃ (66 mg) were taken up in EtCN (10 ml) and heated in a sealed tube at 110° C. for 20 h. The mixture was partitioned between EtOAc and water. The aqueous layer was extracted with EtOAc. The combined organic layers were washed with brine and dried (MgSO₄). Filtration and concentration gave a yellow oil. The residue was purified via thin-layer preparative chromatography (3% EtOAc in DCM, SiO₂) provided 80 mg (59%) of Ex. 1100 as a colorless oil.

### Step 6

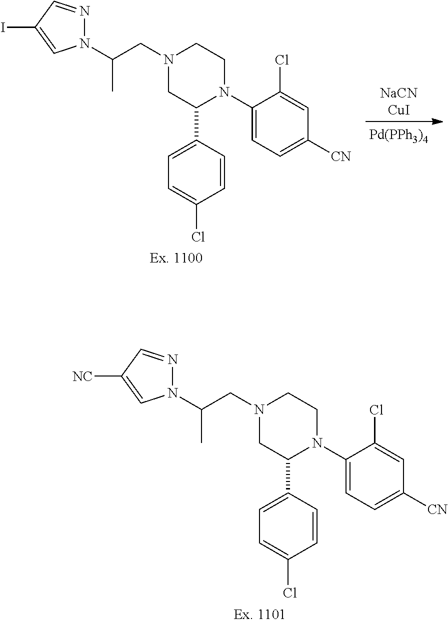

Example 1100 (120 mg, 0.21 mmol), NaCN (15 mg, 0.32 mmol), CuI (4 mg, 0.02 mmol), and Pd(PPh₃)₄ (25 mg, 0.02 mmol) were taken up in CH₃CN (3 ml, degassed) and heat at 90° C. for 45 minutes. The solution was partitioned between 25% NH₄OH and EtOAc. The aqueous layer was extracted with EtOAc. The combined organic layers were washed with brine and dried (MgSO₄). Filtration and concentration gave brown oil. The residue was purified via thin-layer preparative chromatography (5% EtOAc in DCM, SiO₂) which provided 40 mg (41%) of Ex. 1101 as a colorless oil.

### Preparation of Example 1102

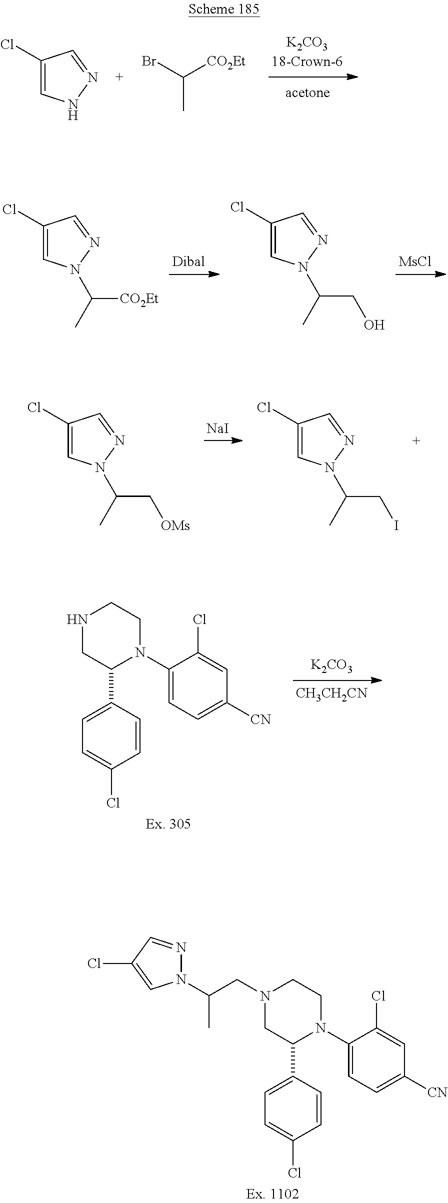

Example 1102 was prepared in a manner similar to that shown in Scheme 184 using the appropriate pyrazole (Scheme 185).

### Preparation of Example 1103

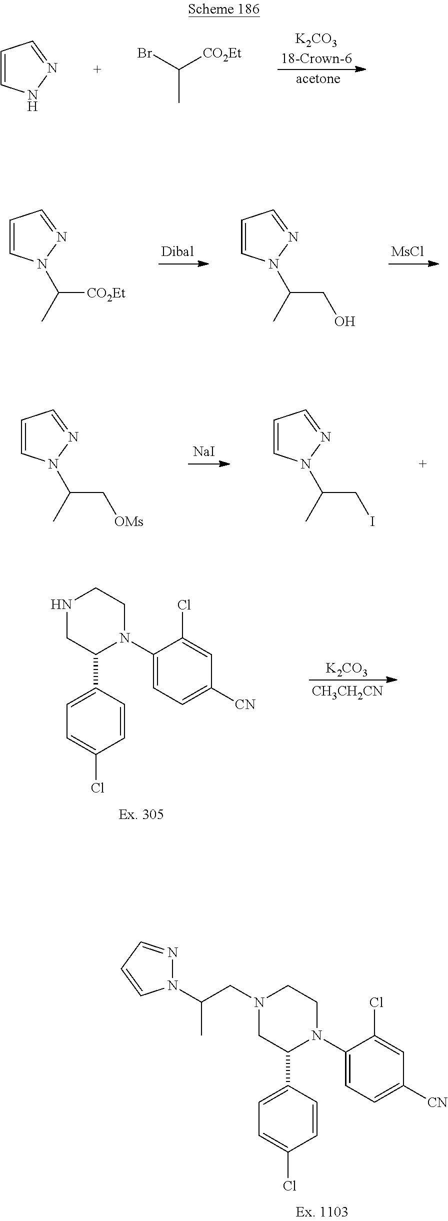

Example 1103 was prepared in a manner similar to that shown in Scheme 184 using the appropriate pyrazole (Scheme 186).

### Preparation of Example 1104a

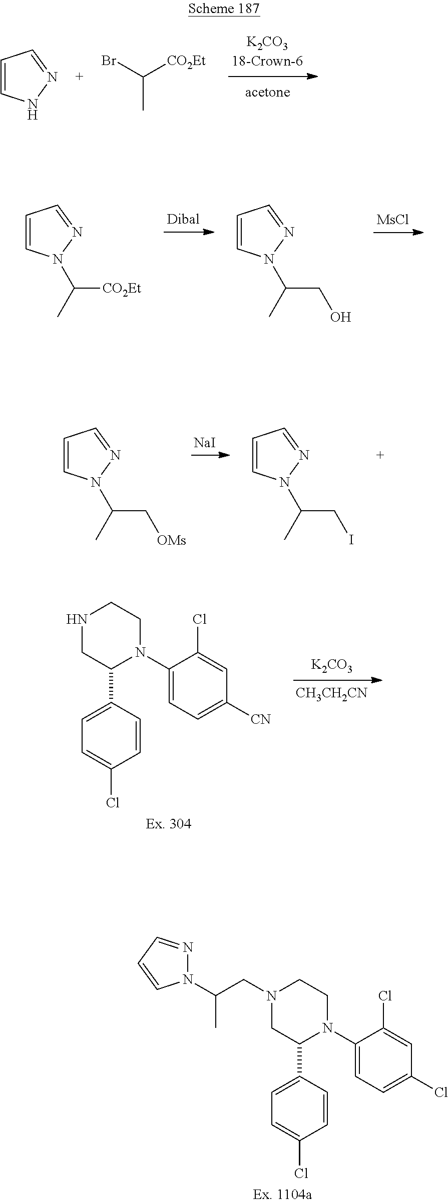

Example 1104a was prepared in a manner similar to that shown in Scheme 184 using Ex. 304 (Scheme 187).

### Preparation of Example 1104b

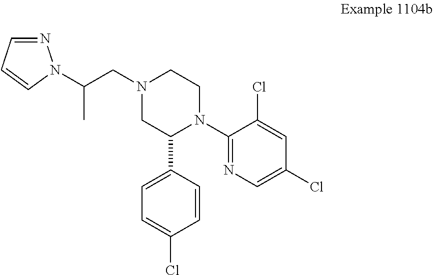 [SMILES: CC(CN1CCN(c2ncc(Cl)cc2Cl)[C@H](c2ccc(Cl)cc2)C1)n1cccn1]

Example 1104b was prepared in the same fashion as Example 1104a except that the piperazine 826c was used instead of Example 304.

### Preparation of Example 1105

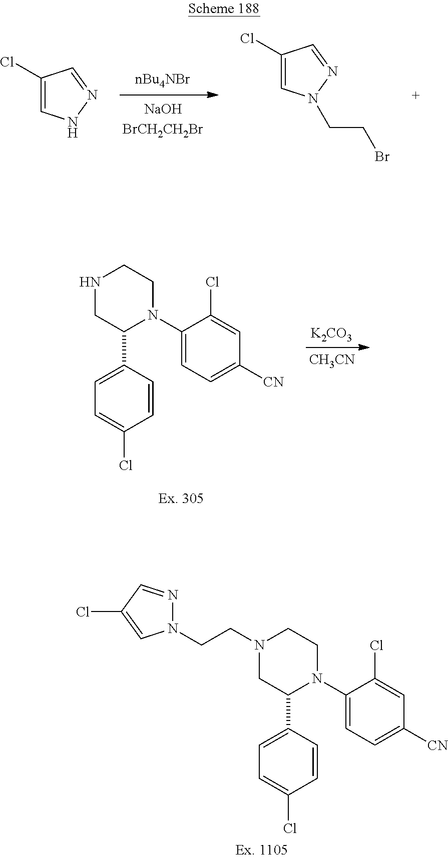

Example 1105 was prepared in a manner similar to that shown in Scheme 184 using the appropriate pyrazole (Scheme 188).

### Preparation of Example 1106

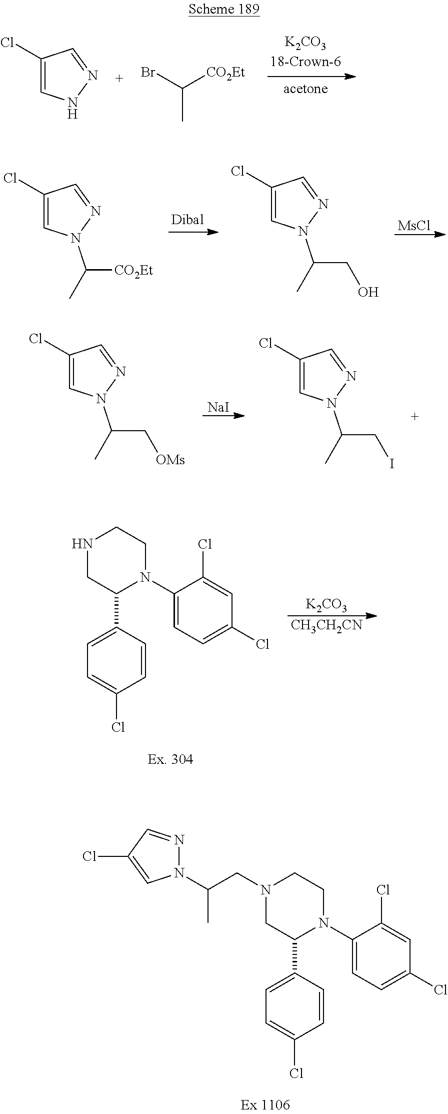

Example 1106 was prepared in a manner similar to that shown in Scheme 184 using Ex. 304 (Scheme 189).

### Preparation of Example 1107

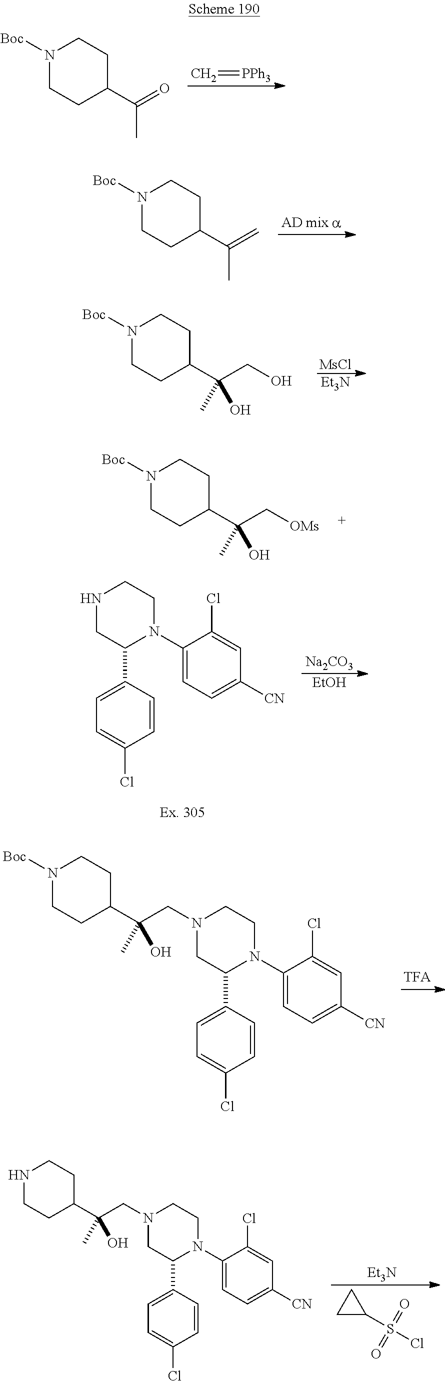

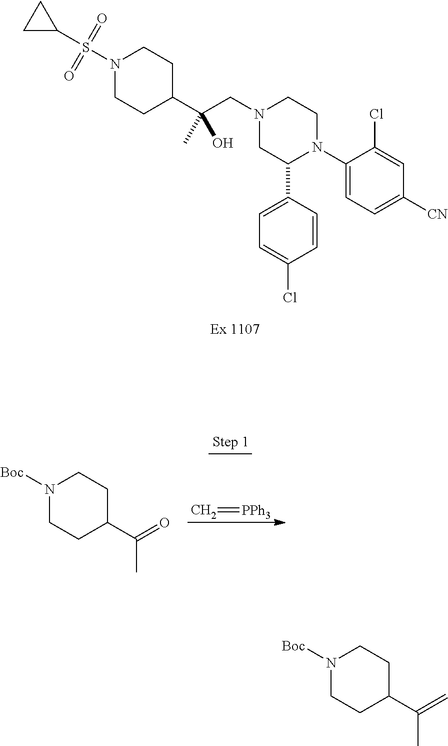

Methyl triphenylphosphonium bromide (18.9 g) was taken up in dry THF (200 ml) under N₂ at 0° C. n-Butyllithium (1.6 M in hexanes, 33 ml) was added dropwise to the solution at 0° C. The resulting red-orange solution was stirred at 0° C. for 2 h. The ketone (8 g, 35.2 mmol) in THF (15 ml) was added at 0° C. The solution was warmed to 25° C. and stirred at that temperature for 4 h. The mixture was partitioned between EtOAc and water. The aqueous layer was extracted with EtOAc. The combined organic layers were washed with brine and dried (MgSO₄). Filtration and concentration gave a yellow semi-solid. The residue was purified via flash chromatography (9/1 hexanes/EtOAc, SiO₂) which provided 4.3 g (54%) of the olefin as a colorless oil.

### Step 2

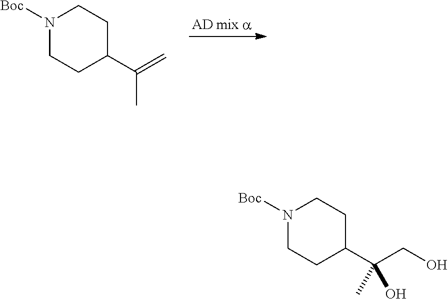 [SMILES: C=C(C)C1CCN(C(=O)OC(C)(C)C)CC1 ; CC(C)(C)OC(=O)N1CCC([C@](C)(O)CO)CC1]

The olefin (4.3 g, 20 mmol) and AD mix α (28 g) were taken up in tert-butanol/water (1/1, 60 ml), and the resulting solution was stirred at 25° C. for 20 h. The solution was cooled to 0° C. and quenched slowly by addition of Na₂SO₃ (28 g) and water (100 ml). The mixture was stirred at 25° C. for 1 h. The mixture was extracted with EtOAc. The combined organic layers were washed with brine and dried (MgSO₄). Filtration and concentration gave the diol as a yellow oil.

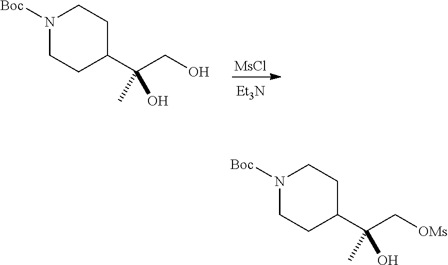 [SMILES: "Et3N" ; "MsCl" ; CC(C)(C)OC(=O)N1CCC([C@](C)(O)CO)CC1 ; CC(C)(C)OC(=O)N1CCC([C@](C)(O)COS(C)(=O)=O)CC1]

### Step 3

The diol (1.2 g) and Et₃N (660 mg) were taken up in DCM (35 ml), and methanesulfonyl chloride (640 mg) was added. The solution was stirred at 25° C. for 0.5 h. The solution was diluted with DCM and washed with sat. NaHCO_{3(aq.)}. The aqueous layer was extracted with DCM. The combined organic layers were dried (MgSO₄), filtered, and concentrated. The mesylate was used without further purification.

### Step 4

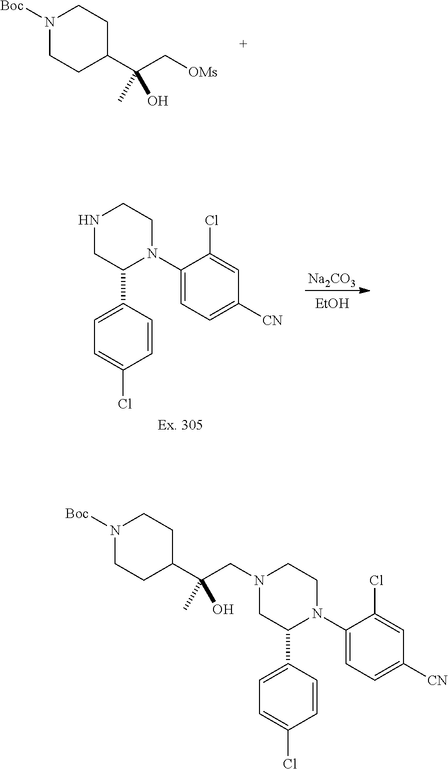

The mesylate (460 mg, 1.36 mmol), Ex. 305 (152 mg, 0.90 mmol), and Na₂CO₃ (300 mg) were taken up in EtOH (10 ml) and heated in a sealed tube at 80° C. for 20 h. The mixture was partitioned between EtOAc and water. The aqueous layer was extracted with EtOAc. The combined organic layers were washed with brine and dried (MgSO₄). Filtration and concentration gave a yellow oil. The residue was purified via thin-layer preparative chromatography (4/1 acetone/DCM, SiO₂) which provided 620 mg (Quant.) of the boc-piperidine as a colorless oil.

### Step 5

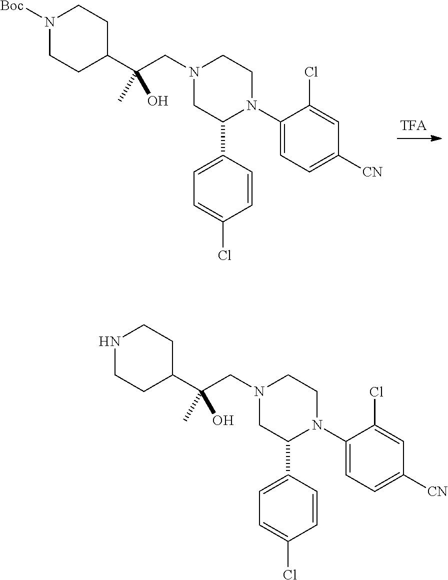 [SMILES: CC(C)(C)OC(=O)N1CCC([C@](C)(O)CN2CCN(c3ccc(C#N)cc3Cl)[C@H](c3ccc(Cl)cc3)C2)CC1 ; C[C@@](O)(CN1CCN(c2ccc(C#N)cc2Cl)[C@H](c2ccc(Cl)cc2)C1)C1CCNCC1]

The boc-piperidine (620 mg, 1.08 mmol) and TFA (6 ml) were taken up in DCM (35 ml), and the resulting solution was stirred at 25° C. for 2 h. The solution was concentrated. The crude TFA salt of the piperidine was used without further purification.

### Step 6

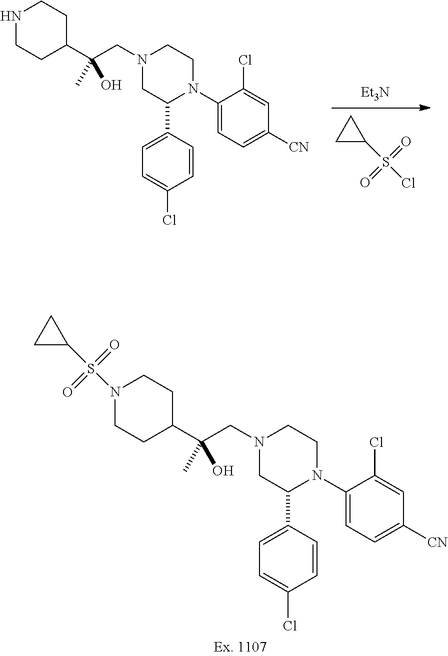

The piperidine-TFA salt (100 mg, 0.21 mmol), Et₃N (43 mg), sulfonyl chloride (35 mg) were taken up in DCM (15 ml), and the resulting solution was stirred at 25° C. for 5 minutes. More Et₃N (90 mg) and sulfonyl chloride (70 mg) were added, and the resulting solution was stirred at 25° C. for 0.5 h. The mixture was diluted with DCM and washed with sat. NaHCO_{3(aq.)}. The aqueous layer was extracted with DCM. The combined organic layers were dried (MgSO₄). Filtration and concentration gave a yellow oil. The residue was purified via flash chromatography (4/1 acetone/DCM, SiO₂) which provided 75 mg (62%) of Ex. 1107 as a colorless oil.

The following examples were prepared in a manner similar to that shown in Scheme 190 using the appropriate conditions for Step 6 (Table XLII).

TABLE XLII

| Condtions for Step 6 | Example | Structure |
|---|---|---|
| 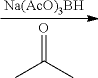 | 1108 | 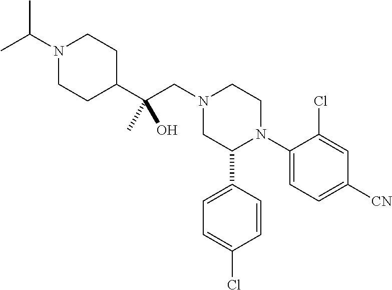 |
| 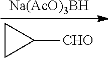 | 1109 | 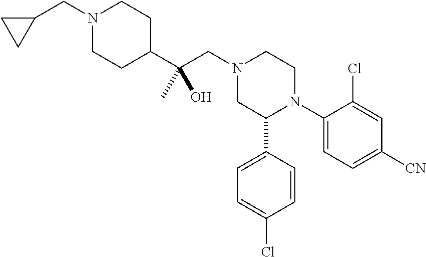 |

### Preparation of Example 1110, 1111 and 1112

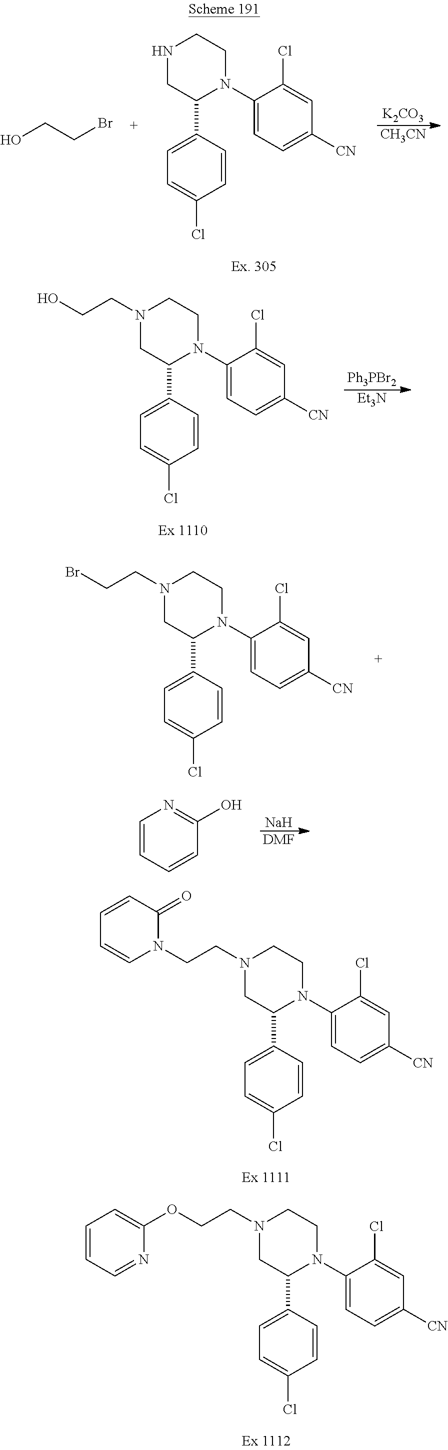

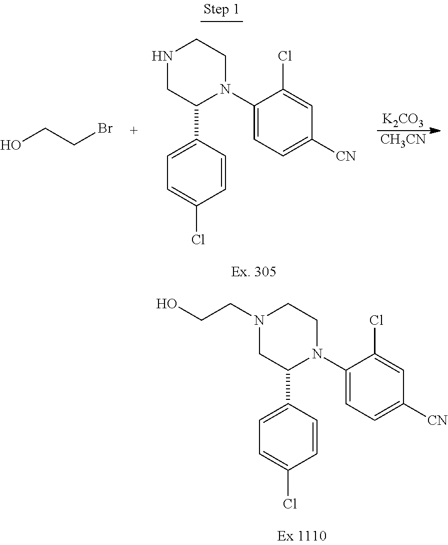

Ex. 305 (300 mg, 0.9 mmol), bromo-ethanol (114 mg) and K₂CO₃ (242 mg) were taken up in CH₃CN (10 ml) and heated at 65° C. for 20 h. More bromo-ethanol (114 mg) was added, and the solution was heated at 65° C. for 18 h. The mixture was partitioned between EtOAc and water. The aqueous layer was extracted with EtOAc. The combined organic layers were washed with brine and dried (MgSO₄). Filtration and concentration gave a yellow oil. The residue was purified via flash chromatography (10% EtOH in DCM, SiO₂) which provided 290 mg (85%) of Ex. 1110 as a colorless oil.

### Step 2

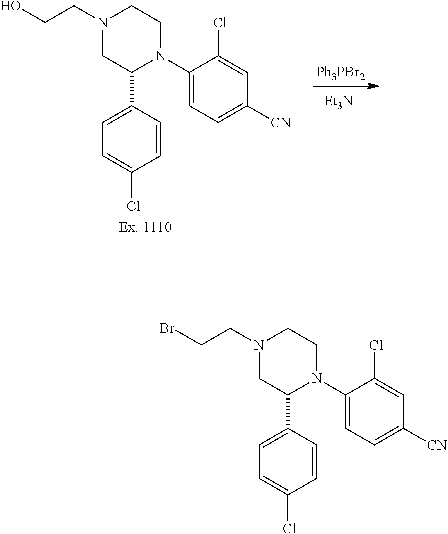 [SMILES: "Et3N" ; N#Cc1ccc(N2CCN(CCO)C[C@H]2c2ccc(Cl)cc2)c(Cl)c1 ; "Ph3PBr2" ; N#Cc1ccc(N2CCN(CCBr)C[C@H]2c2ccc(Cl)cc2)c(Cl)c1]

The alcohol Ex. 1110 (290 mg, 0.77 mmol) and Et₃N (193 mg) were taken up in DCM (8 ml) and cooled to 0° C. Triphenylphosphonium dibromide (490 mg) was added, and the resulting solution was stirred at 25° C. for 1.5 h. The mixture was partitioned between DCM and sat. NaHCO_{3(aq)}. The aqueous layer was extracted with DCM. The combined organic layers were dried (MgSO₄), filtered, and concentrated. The residue was purified via flash chromatography (5% EtOAc in hexanes, SiO₂) which provided 240 mg (71%) of the bromide as a yellow oil.

### Step 3

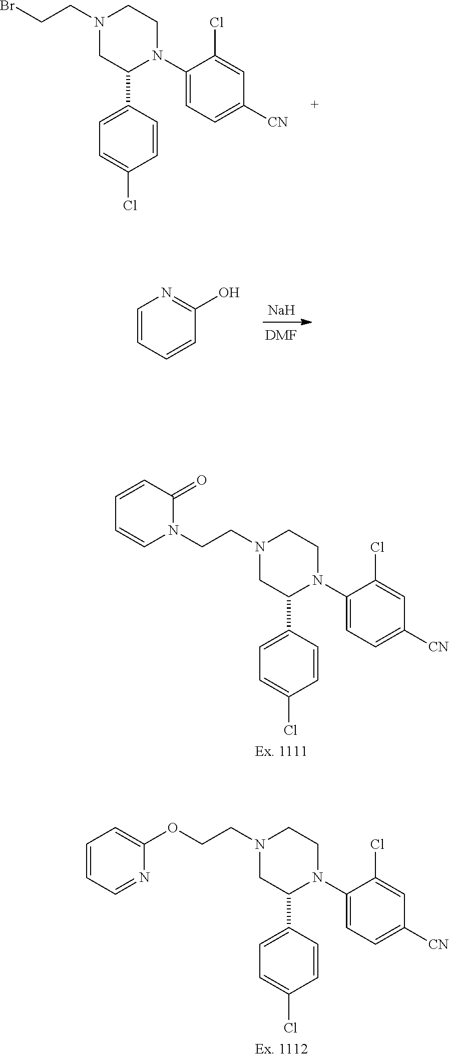

The bromide (82 mg, 0.19 mmol) and 2-hydroxypyridine (35 mg, 0.38 mmol) were taken up in DMF (2 ml), and sodium hydride (29 mg of a 60 wt % dispersion in oil) was added. The mixture was stirred at 25° C. for 2 h. The solution was partitioned between EtOAc and water. The aqueous layer was extracted with EtOAc. The combined organic layers were washed with brine and dried (MgSO₄). Filtration and concentration gave a yellow oil. The residue was purified via thin-layer preparative chromatography (1/1 DCM/acetone, SiO₂) which provided 55 mg (64%) of Ex. 1111 and 10 mg (9%) of Ex. 1112 as a colorless oil.

### Preparation of Example 1113

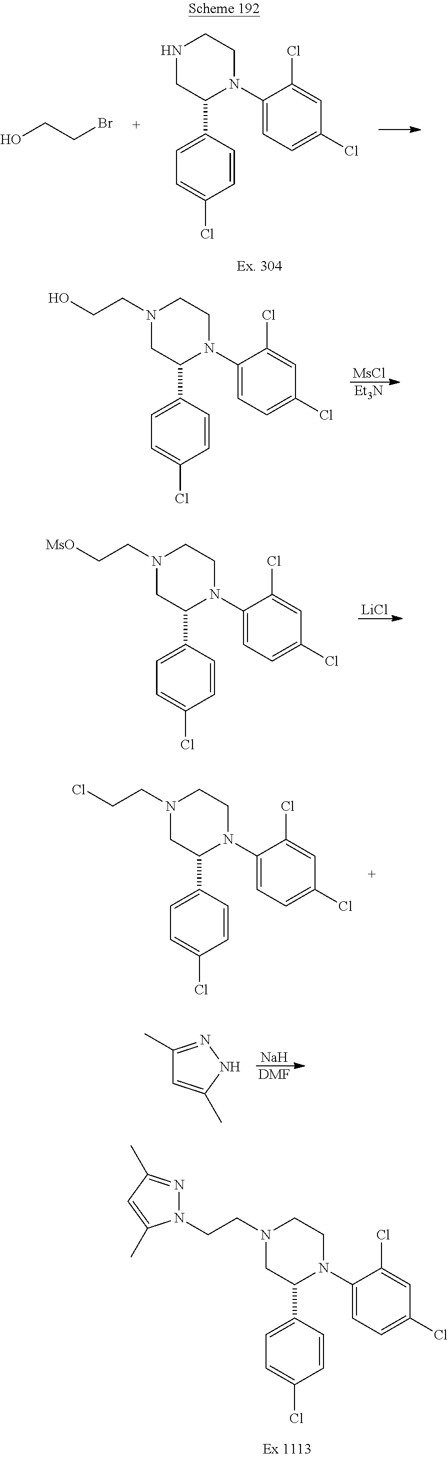

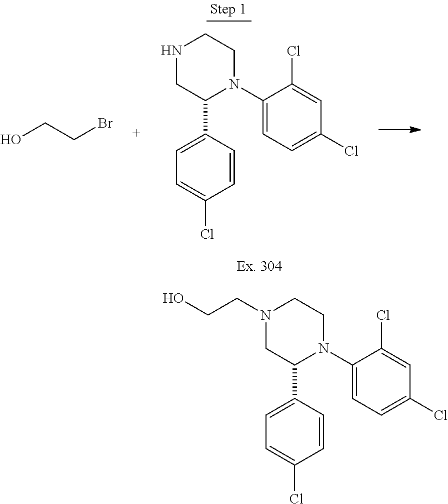

Ex. 304 (1.0 g, 2.92 mmol) was taken up in 2 ml of 2-bromo ethanol, and the solution was heated at 65° C. for 20 h. The solution was partitioned between DCM and 1 N NaOH (aq.). The aqueous layer was extracted with DCM. The combined organic layers were dried (MgSO₄), filtered, and concentrated. The residue was purified via gradient flash chromatography (0-10% MeOH in DCM, SiO₂) which provided 860 mg (76%) of the alcohol as colorless oil.

### Step 2

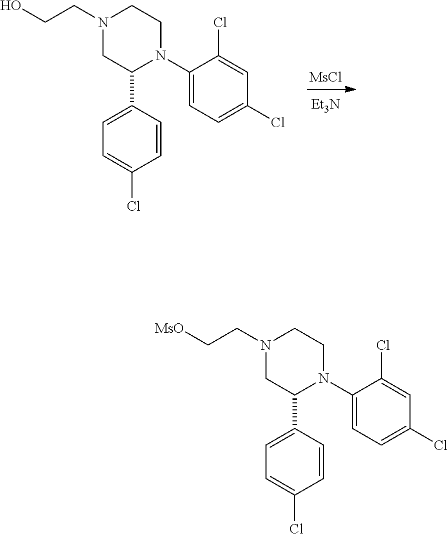 [SMILES: "Et3N" ; "MsCl" ; OCCN1CCN(c2ccc(Cl)cc2Cl)[C@H](c2ccc(Cl)cc2)C1 ; CS(=O)(=O)OCCN1CCN(c2ccc(Cl)cc2Cl)[C@H](c2ccc(Cl)cc2)C1]

The alcohol (860 mg, 2.2 mmol) and Et₃N (0.46 ml) were taken up in DCM (35 ml) and cooled to 0° C. Methansulfonyl chloride (0.2 ml, 2.6 mmol) was added, and the solution was stirred at 25° C. for 2 h. The solution was diluted with DCM and washed with sat. NaHCO_{3(aq.)}. The aqueous layer was extracted with DCM. The combined organic layers were dried (MgSO₄), filtered, and concentrated. The mesylate was used without further purification.

### Step 3

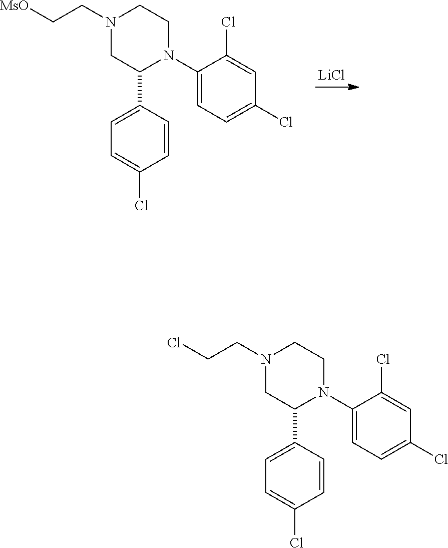 [SMILES: CS(=O)(=O)OCCN1CCN(c2ccc(Cl)cc2Cl)[C@H](c2ccc(Cl)cc2)C1 ; "LiCl" ; ClCCN1CCN(c2ccc(Cl)cc2Cl)[C@H](c2ccc(Cl)cc2)C1]

The mesylate (−2.2 mmol) and LiCl (924 mg) were taken up in CH₃CN, and the resulting solution was heated at 80° C. for 20 h. The solution was partitioned between EtOAc and water. The aqueous layer was extracted with EtOAc. The combined organic layers were washed with brine and dried (MgSO₄). Filtration and concentration gave a yellow oil. The residue was purified via gradient flash chromatography (0-20% EtOAc in hexanes, SiO₂) which provided 60 mg (7%) of the chloride as a yellow oil.

### Step 4

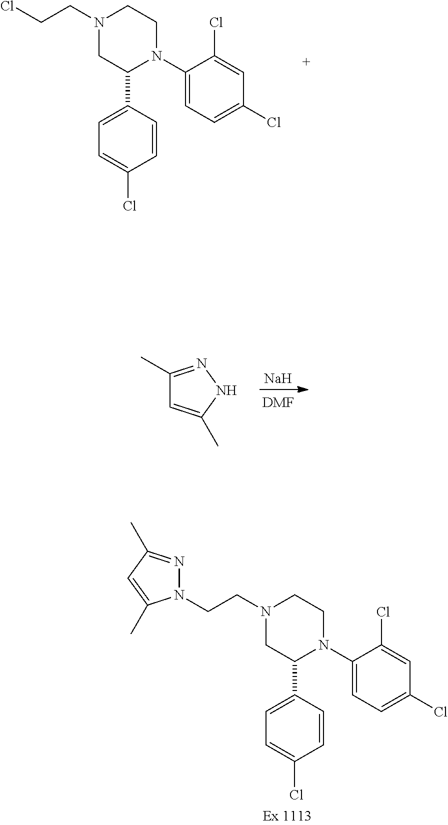

The chloride (60 mg, 0.15 mmol) and pyrazole (30 mg, 0.3 mmol) were taken up in DMF (5 ml). Sodium hydride (20 mg of a 60 wt % dispersion in oil) was added, and the solution was stirred at 25° C. for 20 h. The mixture was partitioned between EtOAc and water. The aqueous layer was extracted with EtOAc. The combined organic layers were washed with brine, dried (MgSO₄), filtered, and concentrated. The residue was purified via thin-layer preparative chromatography (3/1 DCM/acetone, SiO₂) which provided 43 mg (62%) of Ex. 1113 as a colorless oil.

### Preparation of Example 1114

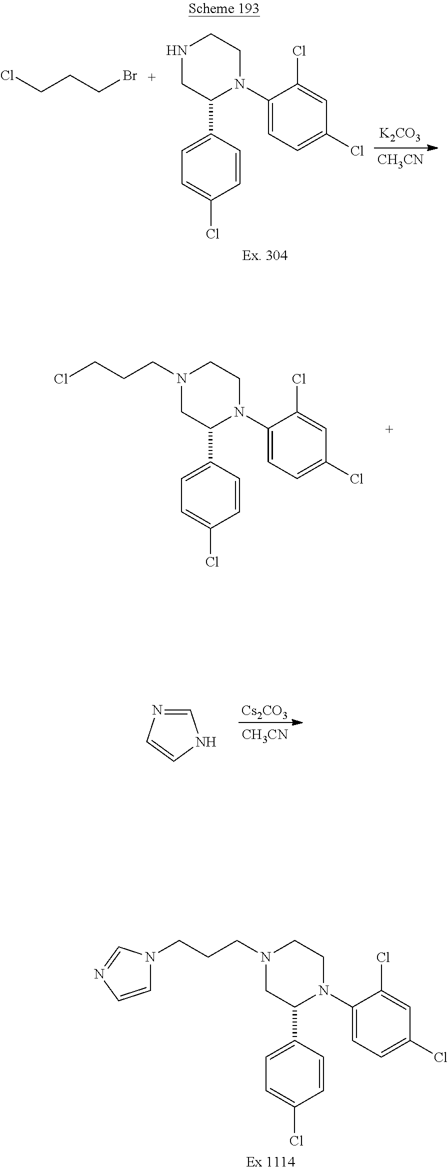

### Step 1

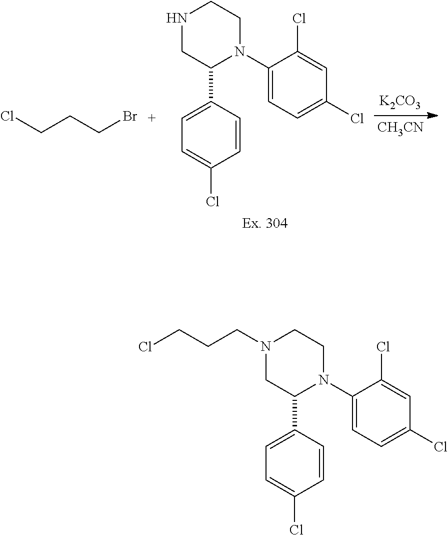

The 3-chlorobromopropane (458 mg), Ex. 304 (500 mg, 1.46 mmol), and K₂CO₃ (500 mg, 3.7 mmol) were taken up in CH₃CN (2 ml), and the resulting mixture was stirred at 25° C. for 20 h. The mixture was partitioned between EtOAc and water. The aqueous layer was extracted with EtOAc. The combined organic layers were washed with brine and dried (MgSO₄), filtered, and concentrated. The residue was purified via gradient flash chromatography (0-30 EtOAc in hexanes, SiO₂) which provided 390 mg (64%) of the chloride as a yellow oil.

### Step 2

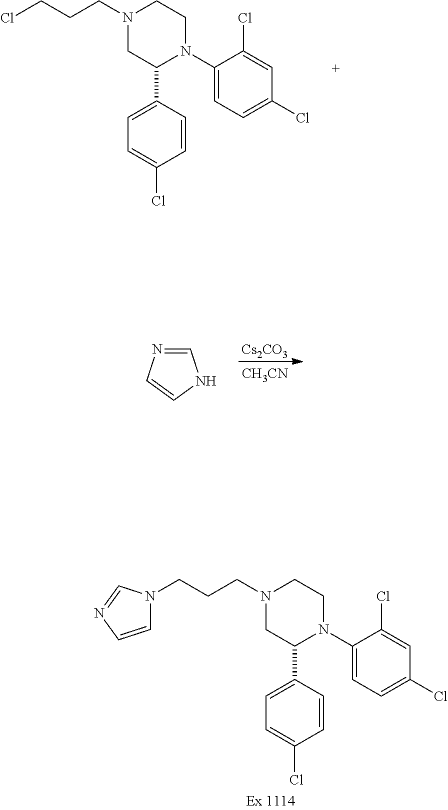

The chloride (100 mg, 0.24 mmol), imidazole (50 mg, 0.72 mmol), and Cs₂CO₃ (234 mg, 0.72 mmol) were taken up in CH₃CN (3 ml), and the resulting mixture was stirred at 85° C. for 20 h. The solution was filtered and concentrated. The residue was purified via thin-layer preparative chromatography (20/1 DCM/EtOH, SiO₂) which provided 108 mg (Quant.) of Ex. 1114 as a colorless oil.

The following examples were prepared in a manner similar to that shown in Scheme 193 using the appropriate heterocyclic reagent in Step 2.

TABLE XLIII

| Heterocyclic Reagent | | |
|---|---|---|
| In Step 2 | Ex | Structure |
| 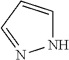 | 1115 | 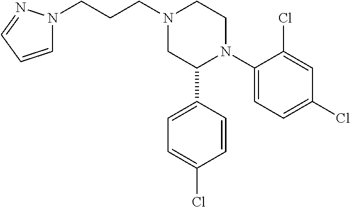 |
| 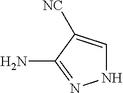 | 1116 | 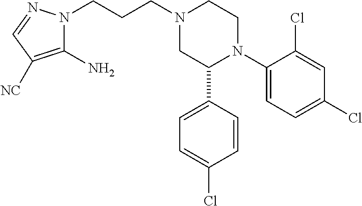 |
| | 1117 | 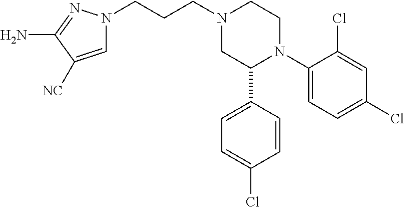 |

### Preparation of Example 1118

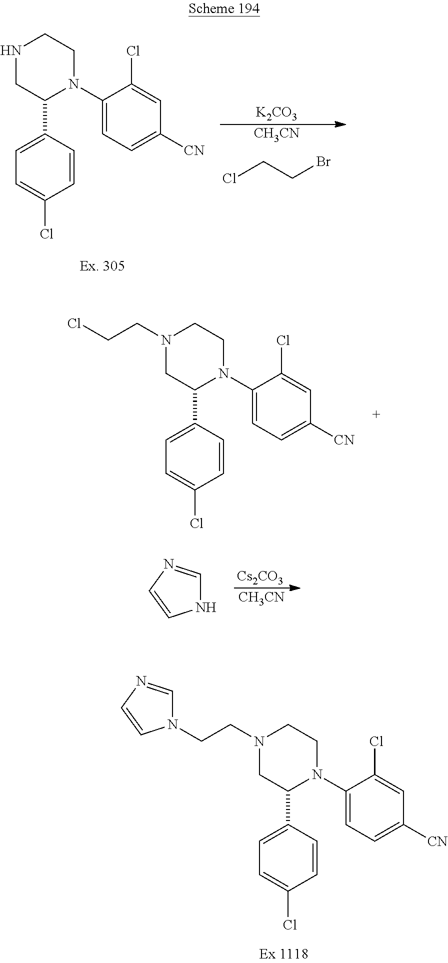

### Step 1

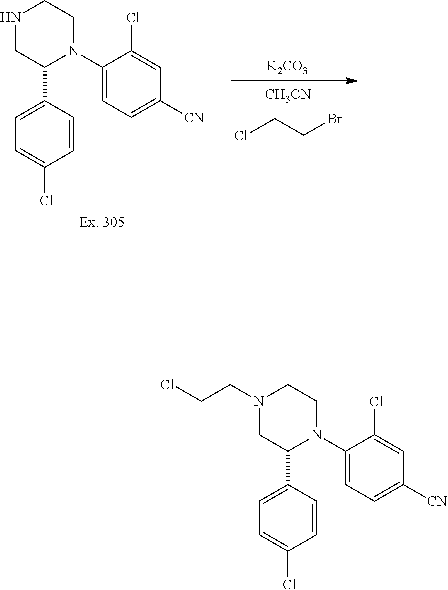

Ex. 305 (500 mg, 1.5 mmol), 2-bromochloroethane (580 mg), and K₂CO₃ (500 mg) were taken up in CH₃CN (2 ml), and the mixture was heated at 75° C. for 18 h. The mixture was partitioned between EtOAc and water. The aqueous layer was extracted with EtOAc. The combined organic layers were washed with brine and dried (MgSO₄). Filtration and concentration gave a yellow oil. The residue was purified via flash chromatography (8% EtOAc in DCM, SiO₂) which provided 210 mg (35%) of the chloride as a yellow oil.

### Step 2

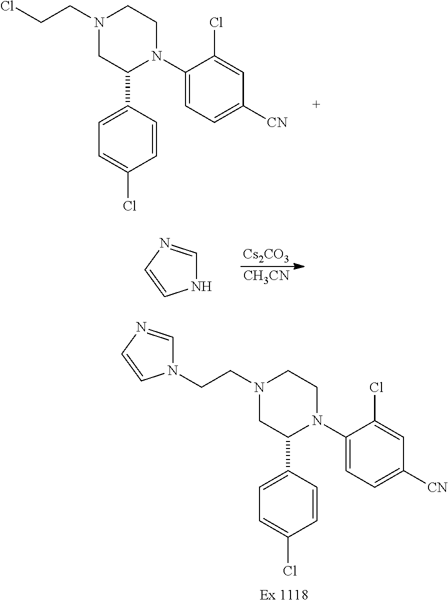

The chloride (70 mg, 0.18 mmol), imidazole (20 mg), and Cs₂CO₃ (115 mg) were taken up in CH₃CN (1.5 ml), and the mixture was heated at 75° C. for 18 h. The mixture was filtered through Celite and concentrated. The residue was purified via thin-layer preparative chromatography (5% EtOH in DCM, SiO₂) which provided 44 mg (53%) of Ex. 1118 as a colorless oil.

The following examples were prepared in a manner similar to that shown in Scheme 194 using the appropriate piperazine and heterocycle depicted in Table XLIV.

TABLE XLIV

| Piperazine | Heterocycle | Ex | Structure |
|---|---|---|---|
| 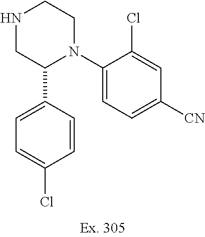 [SMILES: N#Cc1ccc(N2CCNC[C@H]2c2ccc(Cl)cc2)c(Cl)c1] | 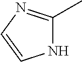 | 1119 | 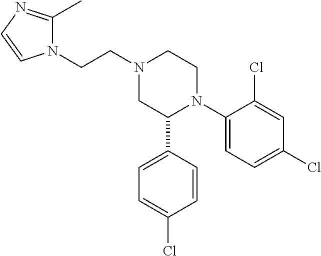 |
| 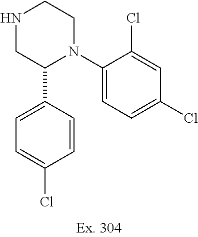 [SMILES: Clc1ccc([C@@H]2CNCCN2c2ccc(Cl)cc2Cl)cc1] | 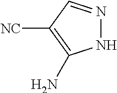 | 1120 | 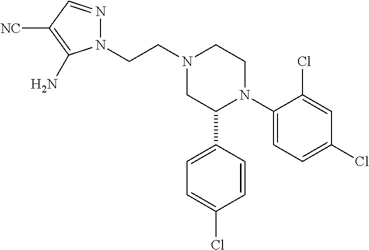 |
| | | 1121 | 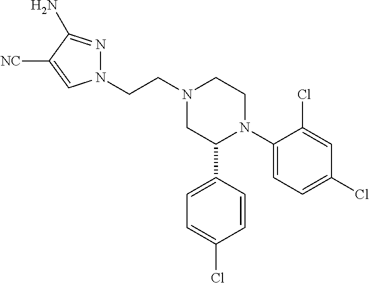 |
| 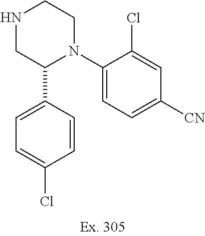 [SMILES: N#Cc1ccc(N2CCNC[C@H]2c2ccc(Cl)cc2)c(Cl)c1] | 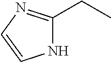 | 1122 | 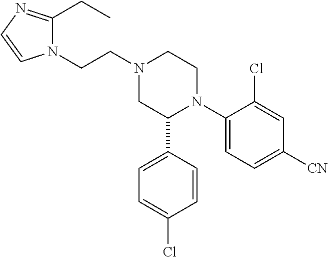 |
| 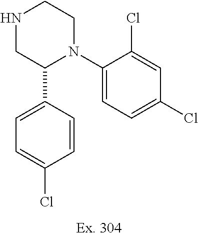 [SMILES: Clc1ccc([C@@H]2CNCCN2c2ccc(Cl)cc2Cl)cc1] | 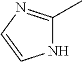 | 1123 | 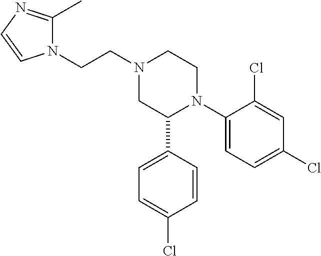 |
| 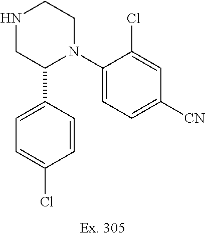 [SMILES: N#Cc1ccc(N2CCNC[C@H]2c2ccc(Cl)cc2)c(Cl)c1] | 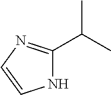 | 1124 | 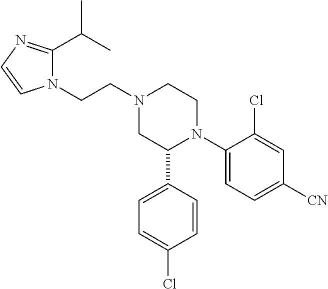 |
| 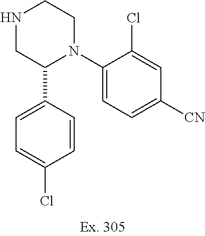 [SMILES: N#Cc1ccc(N2CCNC[C@H]2c2ccc(Cl)cc2)c(Cl)c1] | 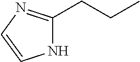 | 1125 | 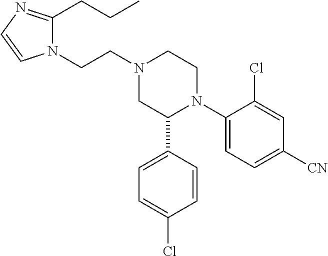 |
| 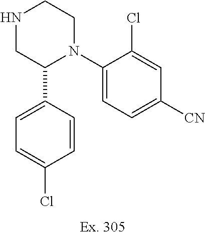 [SMILES: N#Cc1ccc(N2CCNC[C@H]2c2ccc(Cl)cc2)c(Cl)c1] | 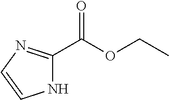 | 1126 | 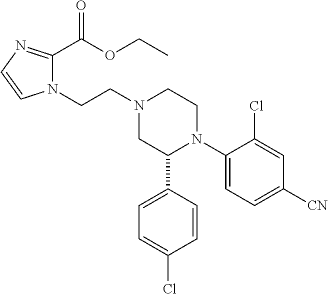 |
| 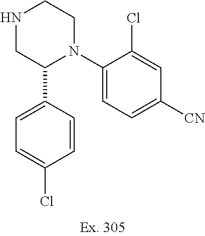 | 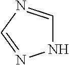 | 1127 | 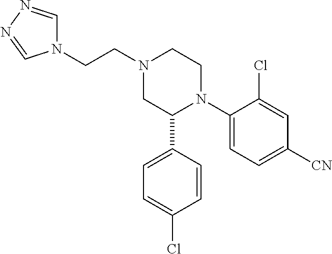 |
| | | 1128 | 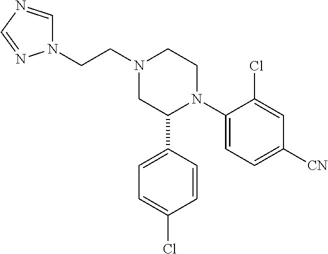 |
| 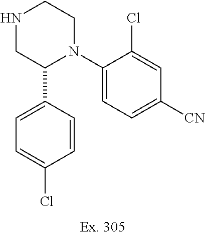 | 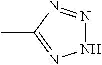 | 1129 | 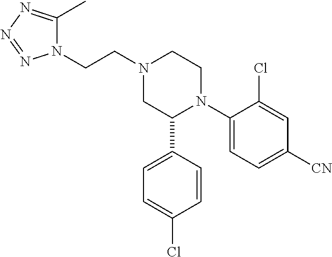 |
| | | 1130 | 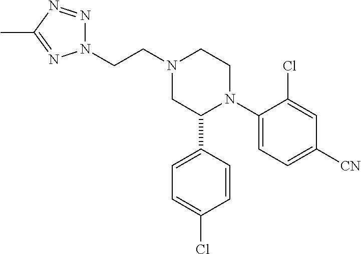 |
| 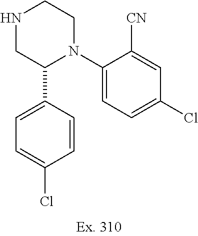 | 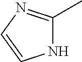 | 1131 | 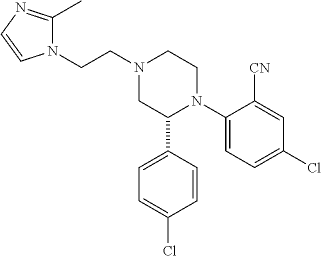 |
| 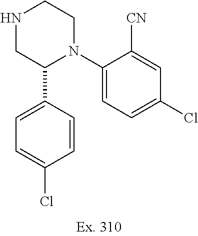 | 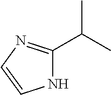 | 1132 | 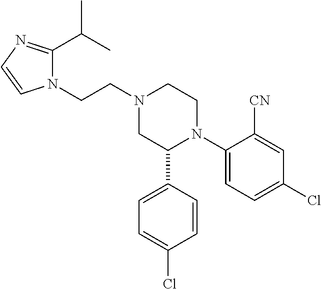 |
| 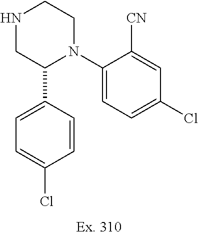 | 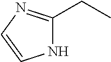 | 1133 | 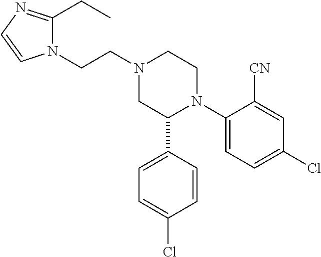 |
| 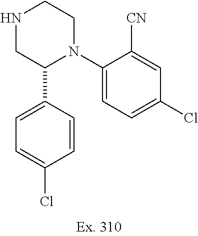 | 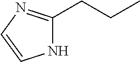 | 1134 | 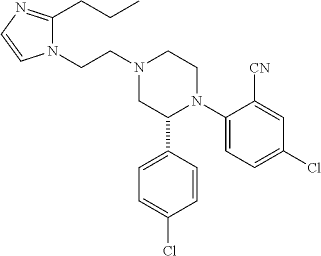 |
| 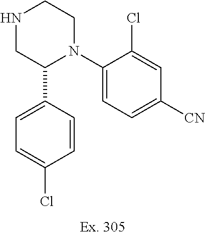 | 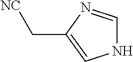 | 1135 | 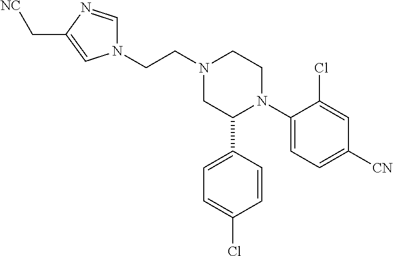 |
| 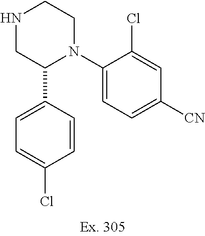 | 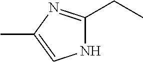 | 1136 | 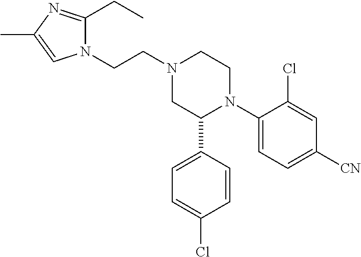 |
| 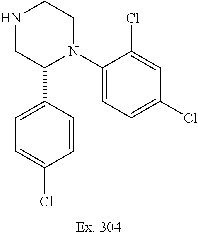 | 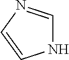 | 1137 | 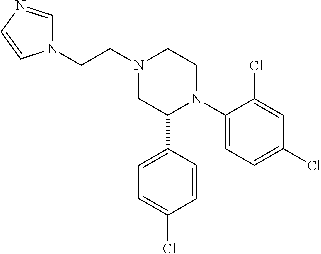 |
| 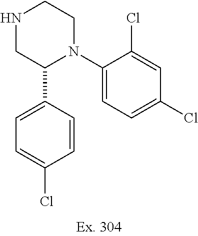 | 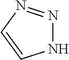 | 1138 | 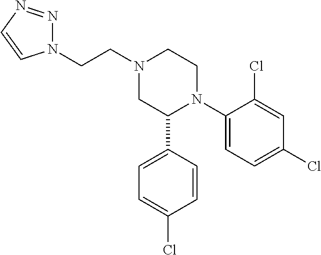 |
| 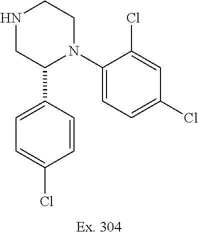 | 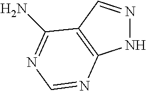 | 1139 | 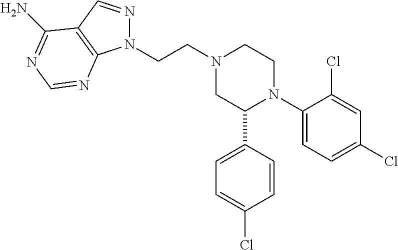 |

The following examples were prepared in a manner similar to that shown in Scheme 192 using the appropriate heterocycle in Step 4.

TABLE XLV

| Heterocyclic Reagent | | |
|---|---|---|
| In Step 4 of Scheme 192 | Ex | Structure |
| 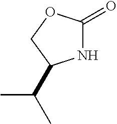 | 1140 | 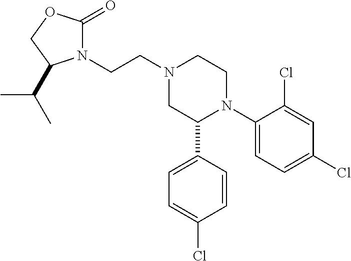 |
| 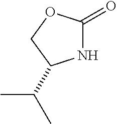 | 1141 | 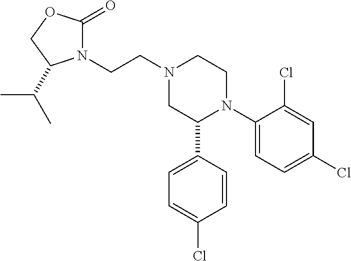 |

### Preparation of Example 1142

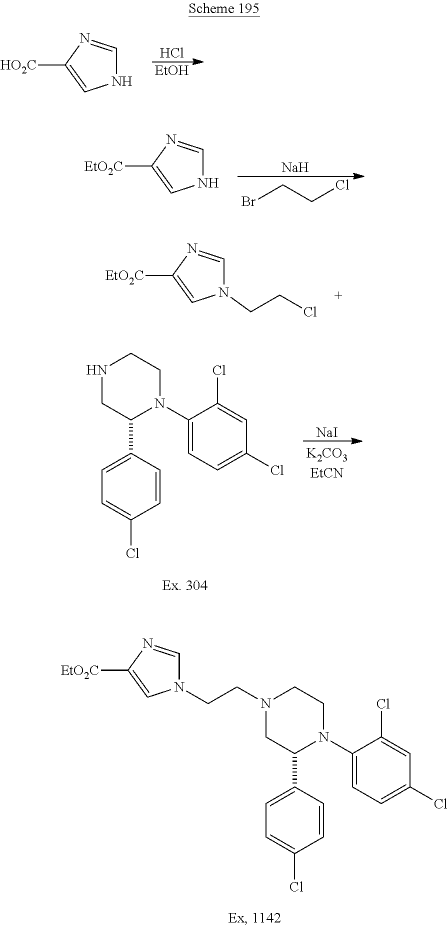

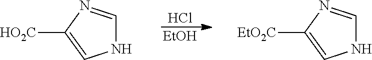 [SMILES: "EtOH" ; O=C(O)c1c[nH]cn1 ; Cl ; CCOC(=O)c1c[nH]cn1]

### Step 1

The imidazole (3.8 g, 33.8 mmol) was taken up in 4.0 M HCl in dioxane (1.7 ml) and EtOH (100 ml). The solution was heated 75° C. for 18 h. The solution was concentrated. The residue was partitioned between EtOAc and sat. NaHCO_{3(aq)}. The aqueous layer was extracted with EtOAc. The combined organic layers were washed with brine and dried (MgSO₄). Filtration and concentration gave 2.5 g (52%) of the ethyl ester as a white solid.

### Step 2

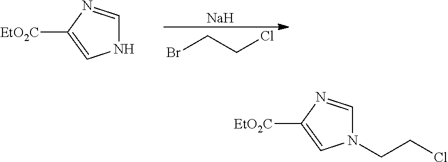

The ethyl ester (500 mg, 3.57 mmol) was suspended in DMF (5 ml). Sodium hydride (171 mg of a 60 wt % dispersion in oil) was added, and the resulting solution was stirred at 25° C. for 0.5 h. 2-Bromochloroethane (0.7 ml, 8.9 mmol) was added, and the resulting solution was stirred at 25° C. for 18 h. The solution was partitioned between EtOAc and water. The aqueous layer was extracted with EtOAc. The combined organic layers were washed with brine and dried (MgSO₄). Filtration and concentration gave a yellow oil. The residue was purified via gradient flash chromatography (0-5% EtOH in DCM, SiO₂) which provided 720 mg (Quant.) of the chloride as a mixture of isomers.

### Step 3

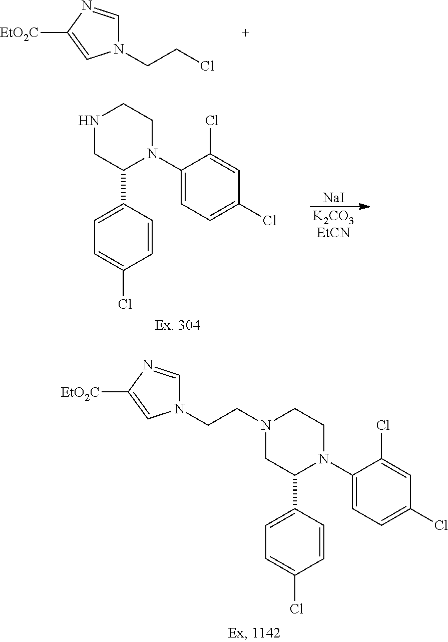

The chloride (˜3.6 mmol), Ex. 304 (500 mg, 1.46 mmol), NaI (219 mg), and K₂CO₃ (604 mg, 4.3 mmol) were taken up in EtCN (3 ml), and the mixture was stirred at 100° C. for 18 h. The mixture was partitioned between EtOAc and water. The aqueous layer was extracted with EtOAc. The combined organic layers were washed with brine and dried (MgSO₄). Filtration and concentration provided a yellow oil. The residue was purified via gradient flash chromatography (0-5% EtOH in DCM, SiO₂). Further purification via thin-layer preparative chromatography (3/1 DCM/acetone, SiO₂) provided 380 mg (51%) of Ex. 1142 as a yellow foam.

### Preparation of Example 1143

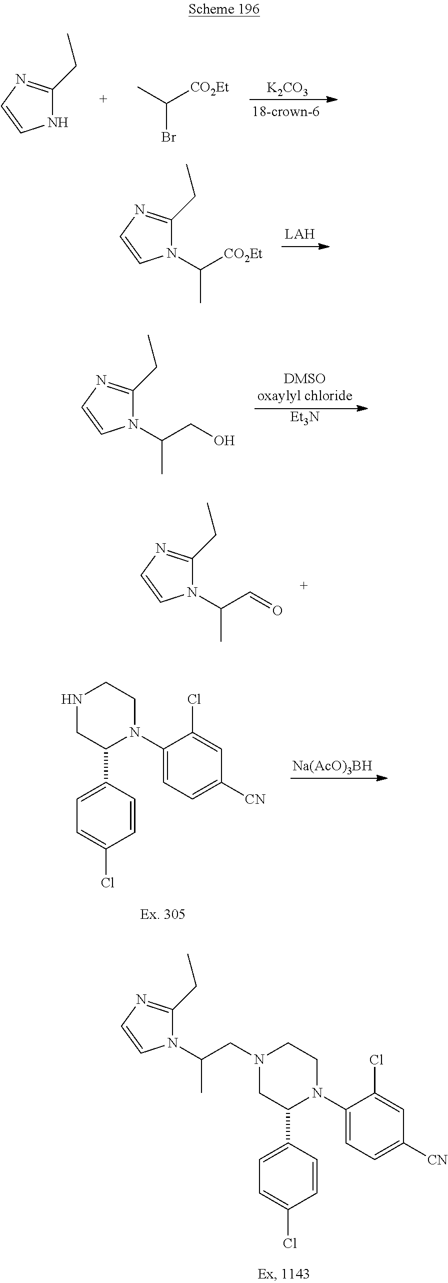

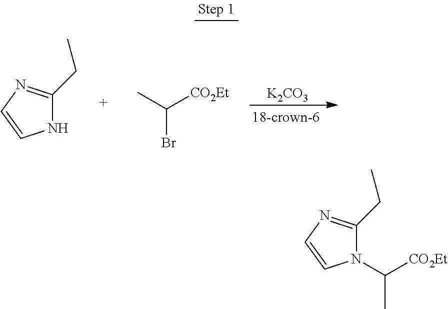

The imidazole (2.8 g, 28.9 mmol), bromo-propionate (5.0 g, 29.2 mmol), K₂CO₃ (7.2 g, 52 mmol), and 18-crown-6 (100 mg) were taken up in acetone (50 ml), and the resulting mixture was stirred at 25° C. for 18 h. The mixture was filtered and concentrated. The residue was partitioned between EtOAc and water. The aqueous layer was extracted with EtOAc. The combined organic layers were washed with brine and dried (MgSO₄). Filtration and concentration provided a yellow oil. The residue was purified via flash chromatography (EtOAc, SiO₂) which provided 2.9 g (51%) of the ester as yellow oil.

### Step 2

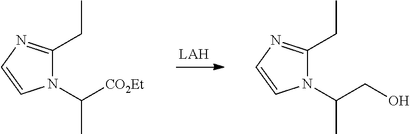

The ester (2.9 g, 14.8 mmol) was taken up in THF (30 ml) and cooled to 0° C. Lithium aluminum hydride (22 ml of a 1.0 M solution in THF) was added dropwise to the solution at 0° C. The solution was allowed to warm to 25° C., and the solution was stirred at 25° C. for 18 h. The solution was cooled to 0° C. and quenched slowly (gas evolution) with 0.8 ml water, 3 N NaOH, and water in that order. The mixture was filtered through Celite and concentrated which provided 2.26 g (99%) of the alcohol as a colorless oil.

### Step 3

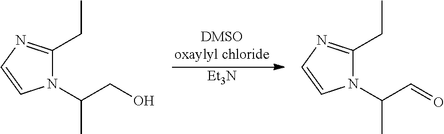

Dimethyl sulfoxide (0.64 ml, 9 mmol) was taken up in DCM (35 ml) at −40° C. (CO₂/CH₃CN). Oxalyl chloride (0.8 ml, 9 mmol) was added dropwise to the DMSO/DCM solution at −40° C. The solution was stirred at −40° C. for 20 minutes. The alcohol (1.08 g, 7 mmol) in DCM (10 ml) was added to the solution at −40° C. The solution was stirred at −40° C. for 20 minutes. Triethylamine (2.9 ml, 21 mmol) was added to the solution at −40° C. The solution was allowed to warm to 25° C., and the resulting solution was stirred at 25° C. for 2 h. The solution was concentrated, and the residue was partitioned between EtOAc and 1 N NaOH_{(aq.)}. The aqueous layer was extracted with EtOAc. The combined organic layers were washed with brine and dried (MgSO₄). The solution was filtered and concentrated which provided the aldehyde as a yellow oil. The material was used without further purification.

### Step 4

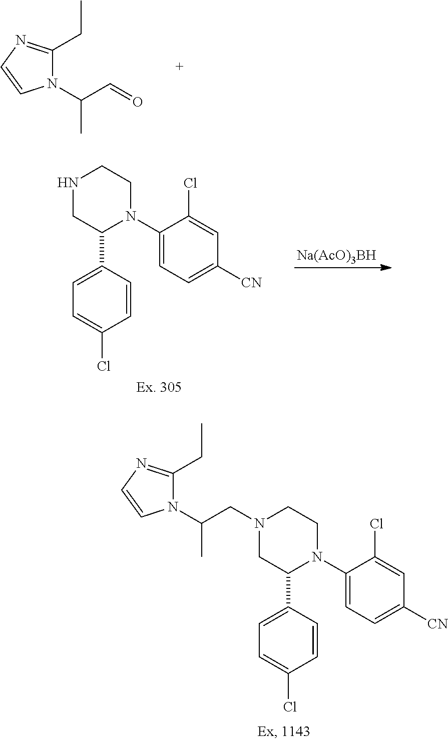

The aldehyde (100 mg, 0.7 mmol), Ex. 305 (180 mg, 0.5 mmol), and Na(AcO)₃BH (160 mg, 0.75 mmol) were taken up in DCM (20 ml) at 25° C. (18 h). The solution was diluted with DCM and washed with 1 N NaOH_{(aq.)}. The aqueous layer was extracted with DCM. The combined organic layers were dried (MgSO₄), filtered, and concentrated. The residue was purified via thin-layer preparative chromatography (20/1 EtOAc/EtOH, SiO₂) which provided 123 mg (53%) of Ex. 1143 as a colorless oil.

The following examples were prepared in a manner similar to that shown in Scheme 196 using the appropriate piperazine in Step 4.

TABLE XLVI

| Piperazine in Step 4 of | | |
|---|---|---|
| Scheme 196 | Ex | Structure |
| 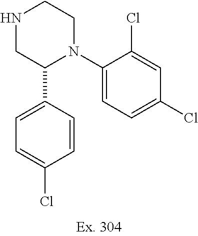 | 1144 | 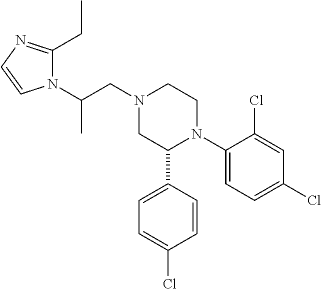 |
| 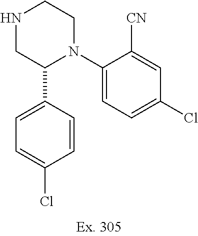 | 1145 | 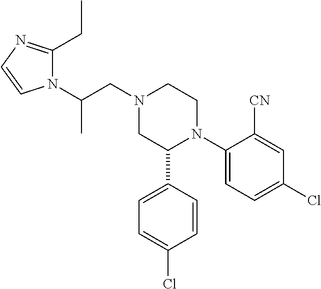 |

### Preparation of Example 1146

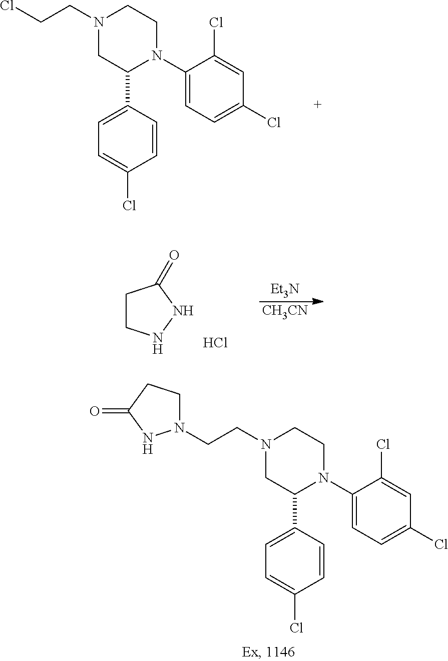 [SMILES: "Et3N" ; ClCCN1CCN(c2ccc(Cl)cc2Cl)[C@H](c2ccc(Cl)cc2)C1 ; O=C1CCNN1 ; "CH3CN" ; Cl ; O=C1CCN(CCN2CCN(c3ccc(Cl)cc3Cl)[C@H](c3ccc(Cl)cc3)C2)N1]

The chloride from Step 3 of Scheme 192 (84 mg, 0.21 mmol), the pyrazolidinone (76 mg, 0.62 mmol), and Et3N (0.2 ml) were taken up in CH₃CN (5 ml) at 85° C. (18 h). The solution was partitioned between EtOAc and 1 N NaOH_{(aq.)}. The aqueous layer was extracted with EtOAc. The combined organic layers were washed with brine and dried (MgSO₄). Filtration and concentration provided a yellow oil. The residue was purified via thin-layer preparative chromatography (21/1 EtOH/DCM, SiO₂) which provided 19 mg (20%) of Ex. 1146 as a white solid.

### Preparation of Example 1147

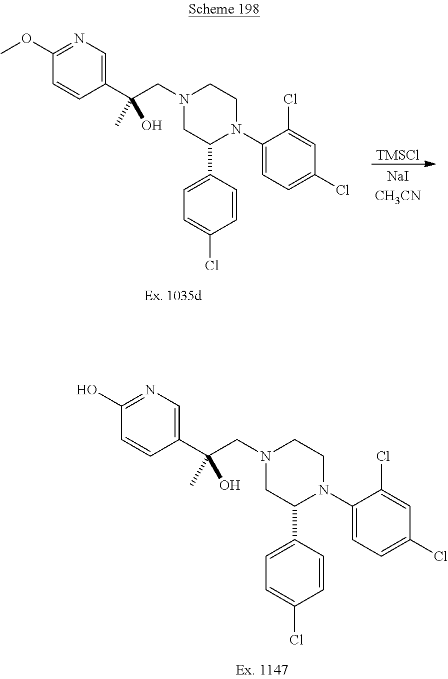 [SMILES: "TMSCl" ; COc1ccc([C@](C)(O)CN2CCN(c3ccc(Cl)cc3Cl)[C@H](c3ccc(Cl)cc3)C2)cn1 ; C[C@@](O)(CN1CCN(c2ccc(Cl)cc2Cl)[C@H](c2ccc(Cl)cc2)C1)c1ccc(O)nc1]

Ex. 1035d (230 mg, 0.45 mmol), TMSCl (480 mg), and NaI (169 mg) were taken up in CH₃CN (8 ml) at 80° C. for 2 h. The solution was partitioned between EtOAc and 10% NH₄OH_{(aq.)}. The aqueous layer was extracted with EtOAc. The combined organic layers were washed with brine and dried (MgSO₄). Filtration and concentration provided a brown oil. The residue was purified via thin-layer preparative chromatography (acetone, SiO₂) which provided 70 mg (29%) of Ex. 1147 as a colorless oil.

The following examples were prepared in a manner similar to that shown in Scheme 198 using the appropriate alkoxy heterocycle.

TABLE XLVII

| Piperazine in Step 4 of Scheme | | |
|---|---|---|
| 32 | Ex | Structure |
| 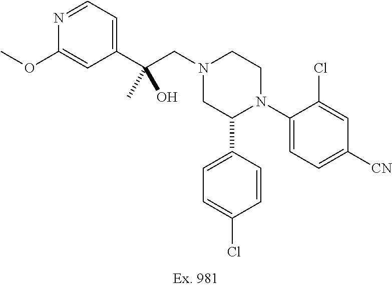 | 1148 | 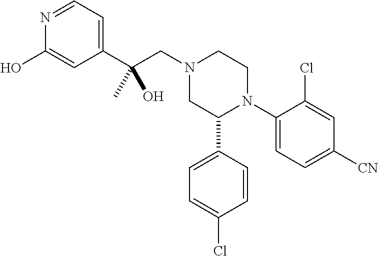 |
| 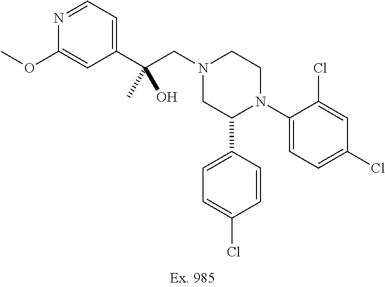 | 1149 | 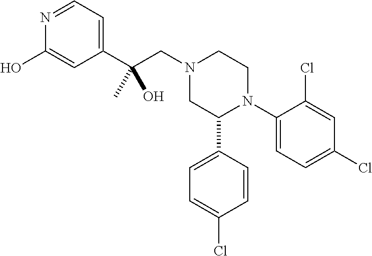 |
| 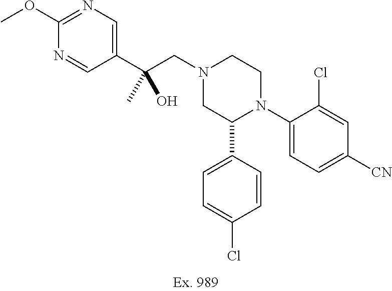 | 1150 | 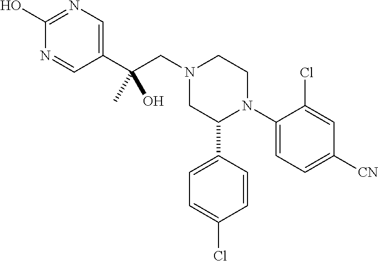 |
| 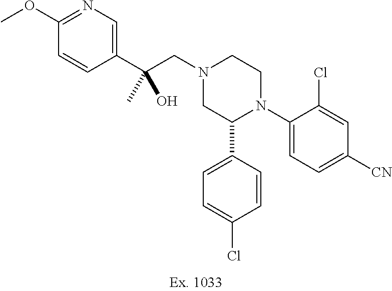 | 1151 | 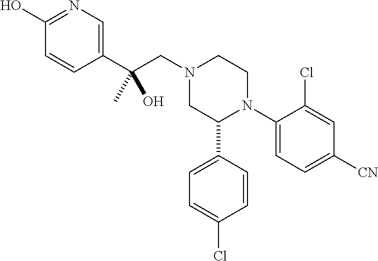 |

### Preparation of Example 1152 and 1153

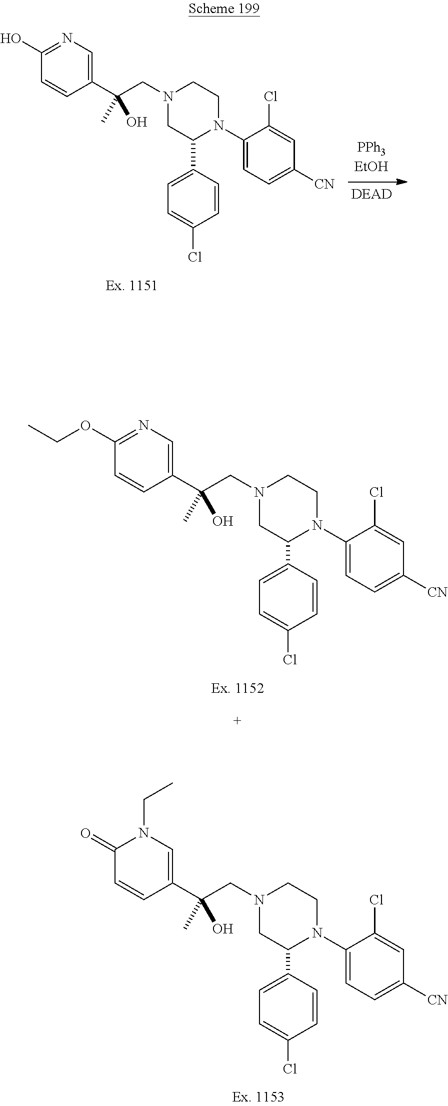 [SMILES: C[C@@](O)(CN1CCN(c2ccc(C#N)cc2Cl)[C@H](c2ccc(Cl)cc2)C1)c1ccc(O)nc1 ; CCOc1ccc([C@](C)(O)CN2CCN(c3ccc(C#N)cc3Cl)[C@H](c3ccc(Cl)cc3)C2)cn1 ; CCn1cc([C@](C)(O)CN2CCN(c3ccc(C#N)cc3Cl)[C@H](c3ccc(Cl)cc3)C2)ccc1=O]

Ex. 1151 (127 mg, 0.26 mmol), PPh₃ (103 mg), DEAD (68 mg), and EtOH (145 mg) were taken up in DCM (2 ml) at 25° C. for 18 h. The solution was concentrated. The residue was purified via thin-layer preparative chromatography (40% EtOAc in DCM, SiO₂) which provided 13 mg (9%) of Ex. 1152 and 51 mg (36%) of Ex. 1153 as colorless oils.

Example 1154 was prepared in a similar fashion to that shown in Scheme 199.

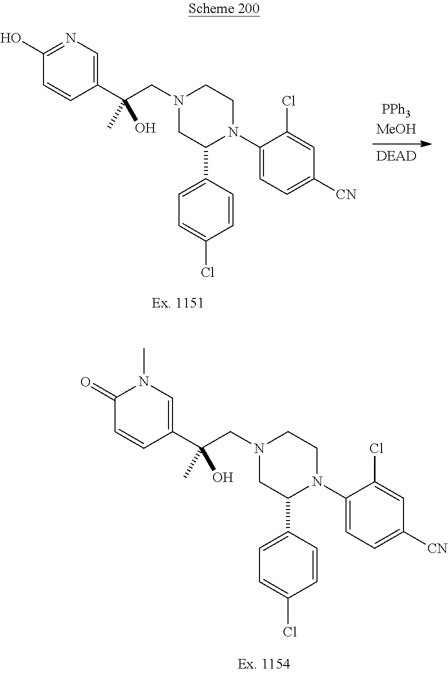 [SMILES: C[C@@](O)(CN1CCN(c2ccc(C#N)cc2Cl)[C@H](c2ccc(Cl)cc2)C1)c1ccc(O)nc1 ; Cn1cc([C@](C)(O)CN2CCN(c3ccc(C#N)cc3Cl)[C@H](c3ccc(Cl)cc3)C2)ccc1=O]

### Preparation of Example 1155a

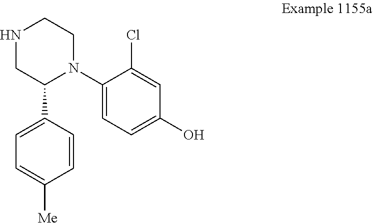 [SMILES: Cc1ccc([C@@H]2CNCCN2c2ccc(O)cc2Cl)cc1]

Example 1155a was prepared from Example 811 in a manner similar to that described for the synthesis of Example 813 in Scheme 95.

The following examples were prepared in a similar manner to that described in Scheme 38.

TABLE XLVIII

| | | Ex- | |
|---|---|---|---|
| | | ample | |
| Piperazine | Alcohol | # | Example Structure |
| 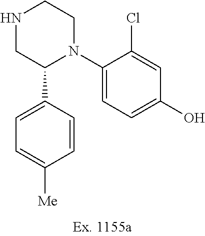 [SMILES: Cc1ccc([C@@H]2CNCCN2c2ccc(O)cc2Cl)cc1] | 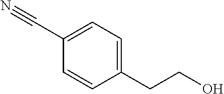 [SMILES: N#Cc1ccc(CCO)cc1] | 1155b | 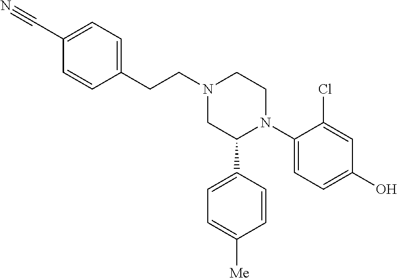 [SMILES: Cc1ccc([C@@H]2CN(CCc3ccc(C#N)cc3)CCN2c2ccc(O)cc2Cl)cc1] |
| 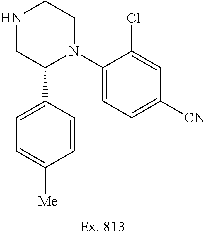 [SMILES: Cc1ccc([C@@H]2CNCCN2c2ccc(C#N)cc2Cl)cc1] | 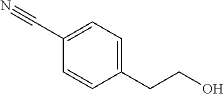 [SMILES: N#Cc1ccc(CCO)cc1] | 1156 | 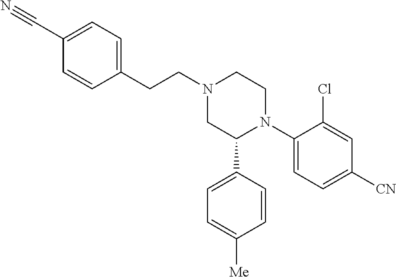 [SMILES: Cc1ccc([C@@H]2CN(CCc3ccc(C#N)cc3)CCN2c2ccc(C#N)cc2Cl)cc1] |

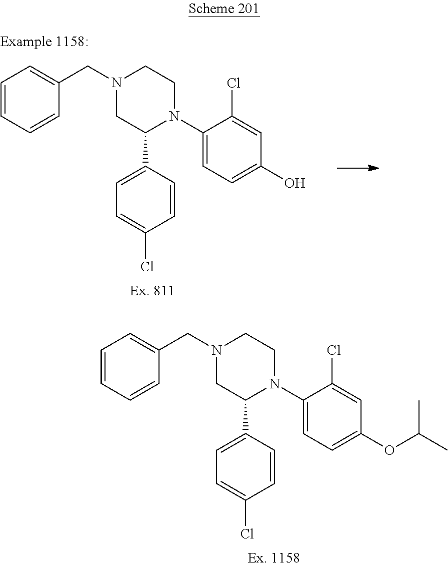 [SMILES: Oc1ccc(N2CCN(Cc3ccccc3)C[C@H]2c2ccc(Cl)cc2)c(Cl)c1 ; CC(C)Oc1ccc(N2CCN(Cc3ccccc3)C[C@H]2c2ccc(Cl)cc2)c(Cl)c1]

To a solution of Ex. 811 (500 mg, 1.21 mmol, 1 eq), pentaerythritol (823 mg, 6.05 mmol, 5 eq.), and triphenylphosphine (477 mg, 1.82 mmol, 1.5 eq) in THF (5 mL) at 0° C. was added diisopropylazodicarboxylate (357 μL, 1.82 mmol, 1.5 eq). The resulting mixture was stirred for 16 h, allowing the ice bath to expire. Isopropanol was added to the reaction, and the resulting mixture was stirred for 72 h. The reaction was then poured into 1:1 hexanes:EtOAc and the resulting mixture was stirred. A solid resulted which was removed via filtration. The resulting filtrate was concentrated and chromatographed (SiO₂, gradient elution, 0% to 100% EtOAc in hexanes) to afford Ex. 1158 (400 mg, 73%).

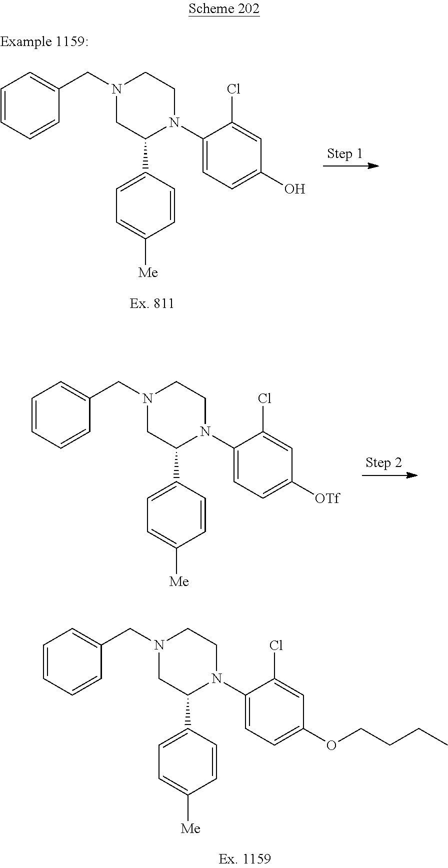 [SMILES: Cc1ccc([C@@H]2CN(Cc3ccccc3)CCN2c2ccc(O)cc2Cl)cc1 ; Cc1ccc([C@@H]2CN(Cc3ccccc3)CCN2c2ccc(OS(=O)(=O)C(F)(F)F)cc2Cl)cc1 ; CCCCOc1ccc(N2CCN(Cc3ccccc3)C[C@H]2c2ccc(C)cc2)c(Cl)c1]

### Step 1:

A solution of Ex. 811 (500 mg, 1.27 mmol, 1 eq) in pyridine (5 mL) at 0° C. under a nitrogen atmosphere was treated with trifluoromethanesulfonic anhydride (225 μL, 1.34 mmol, 1.05 eq). The resulting mixture was stirred 1 h at 0° C. The reaction mixture was then partitioned between EtOAc and brine. The aqueous layer was discarded and the organic layer was washed with brine, dried over anhydrous MgSO₄, filtered, and evaporated to afford a crude residue. Silica gel chromatography (gradient elution 0% to 80% EtOAc in hexanes) afforded the aryl triflate (491 mg, 74%) as a pale yellow, viscous oil.

### Step 2:

The aryl triflate prepared in Step 1 (50 mg, 0.095 mmol, 1 eq), n-butanol (40 μL, 0.44 mmol, 4.6 eq), and Cs₂CO₃ (49 mg, 0.15 mmol, 1.6 eq) were added to o-xylene (0.5 mL). The resulting mixture was heated 3 h at 130° C. The solvent was removed in vacuo at 130° C. to afford a crude residue which was purified via silica gel chromatography (gradient elution, 0% to 70% EtOAc in hexanes) to afford Ex. 1159 (21 mg, 50% yield) as a clear film.

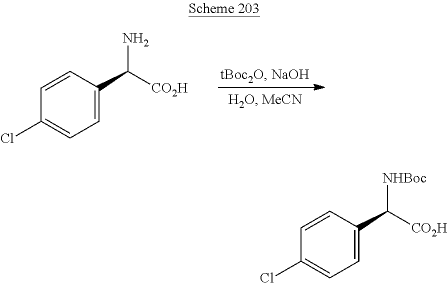 [SMILES: N[C@@H](C(=O)O)c1ccc(Cl)cc1 ; CC(C)(C)OC(=O)N[C@@H](C(=O)O)c1ccc(Cl)cc1]

A solution of (R)-2-amino-2-(4-chlorophenyl)acetic acid (1.0 g, 5.4 mmol), di-tert-butyl dicarbonate (1.2 g, 5.4 mmol) and NaOH (450 mg, 11 mmol) in water and MeCN (4:3) was stirred at RT overnight. The solution was acidified by the addition of 1 N HCl (aq.). The solution was extracted with CH₂Cl₂. The organic layer was dried over Na₂SO₄, filtered and concentrated in vacuo to afford the acid (1.4 g).

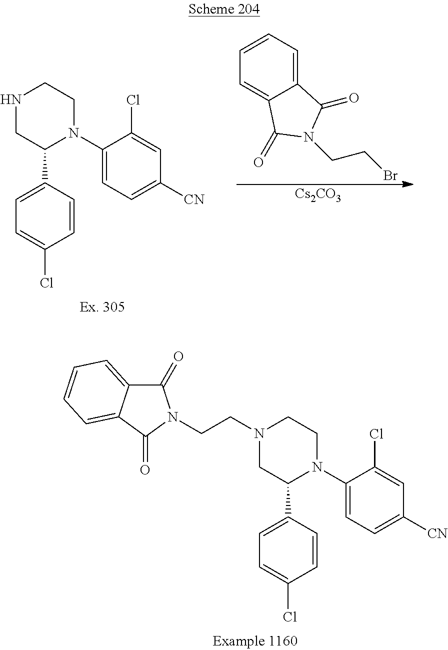

To a solution of the bromide (2.7 g, 10.8 mmol) and Ex. 305 (3 g, 9.0 mmol) in MeCN in a pressure tube was added Cs₂CO₃ (4 g). The pressure tube was sealed and the mixture was heated to 80° C. with stirring. After 16 h, the mixture was cooled to RT and concentrated in vacuo. The residue was then partitioned between CH₂Cl₂ and water. The aqueous layer was extracted with CH₂Cl₂ (3×). The combined organic layers were dried over Na₂SO₄, filtered and concentrated. The crude product was purified via flash chromatography (SiO₂: gradient elution 100:0 to 1:1 hexanes:EtOAc) to afford Example 1160 (1.42 g) as a light yellow solid: LCMS (MH⁺) 505.3.

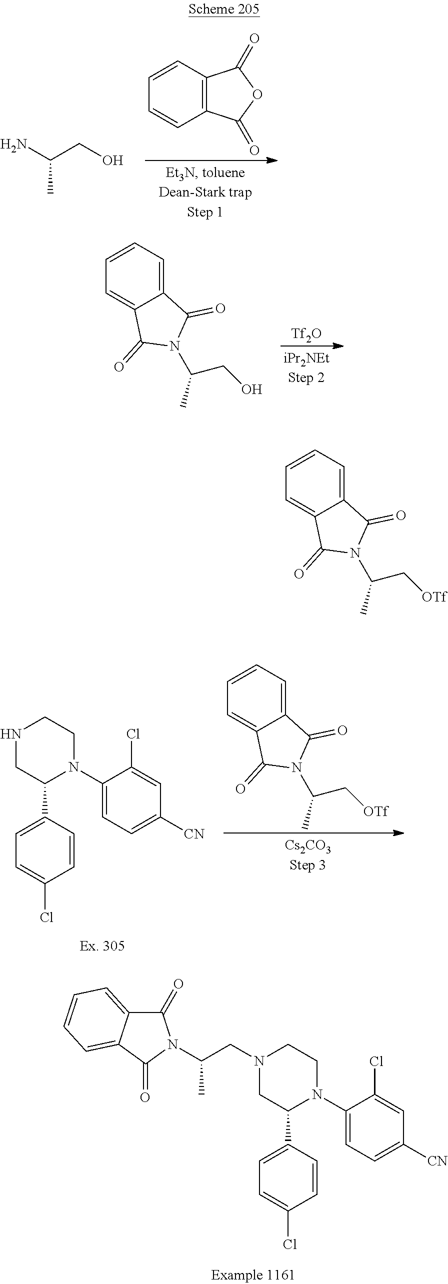

### Step 1:

To a solution of 2-(S)-amino-1-propanol (2.0, 27 mmol) in toluene in a round bottom flask was added Et₃N (0.37 mL, 2.65 mmol) and phthalic anhydride (3.9 g, 27 mmol). A Dean-Stark trap was attached and the solution was heated to reflux for 24 h with stirring. After the reaction was determined to be complete, the solution was concentrated in vacuo and the crude product was purified via flash chromatography (SiO₂: gradient elution 100:0 to 0:100 hexanes:EtOAc) to afford the alcohol (4.9 g) as a white solid.

### Step 2:

To a solution of the alcohol from step 1 (500 mg, 2.4 mmol) in CH₂Cl₂ at −25° C. was added iPr₂NEt (465 mg, 3.6 mmol) followed by trifluoromethanesulfonic anhydride (745 mg, 2.6 mmol). The resultant solution was stirred at −25° C. for 1 h. After that time, the solution was concentrated and the residue was filtered through a silica gel plug using 1:1 EtOAc:hexanes as the eluant. The solvent was concentrated to afford the triflate (ca 300 mg).

### Step 3:

To a solution of Ex. 305 (500 mg) in DMF (5 mL) at 0° C. was added the triflate from step 2 (ca 300 mg) followed by iPr₂NEt (0.6 mL). The resultant solution was allowed to slowly warm to RT and stir for 48 h. The solution was then concentrated in vacuo and the residue was partitioned between water and CH₂Cl₂. The organic layer was dried over Na₂SO₄, filtered and concentrated. The crude product was purified via flash chromatography (SiO₂: gradient elution 100:0 to 50:50 hexanes:EtOAc) to afford Example 1161 (470 mg) as a light yellow solid: LCMS (MH⁺) 519.3.

The following amino alcohols were converted to the following examples using a method similar to that described in Scheme 205.

TABLE XLIX

| Amino alcohol | Ex | Piperazine | Ex | Structure |
|---|---|---|---|---|
| 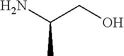 | 305 | 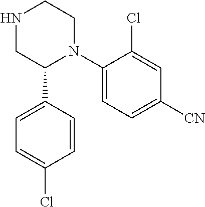 [SMILES: N#Cc1ccc(N2CCNC[C@H]2c2ccc(Cl)cc2)c(Cl)c1] | 1162 | 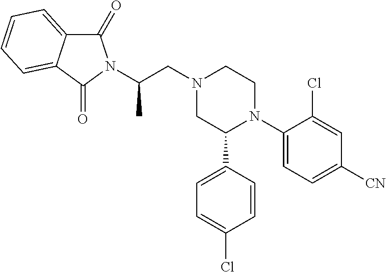 [SMILES: C[C@H](CN1CCN(c2ccc(C#N)cc2Cl)[C@H](c2ccc(Cl)cc2)C1)N1C(=O)c2ccccc2C1=O] |
| 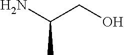 | 304 | 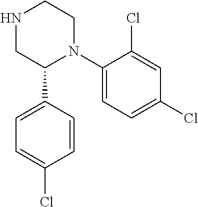 [SMILES: Clc1ccc([C@@H]2CNCCN2c2ccc(Cl)cc2Cl)cc1] | 1163 | 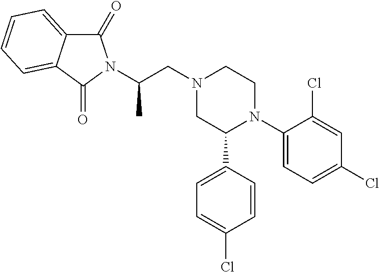 [SMILES: C[C@H](CN1CCN(c2ccc(Cl)cc2Cl)[C@H](c2ccc(Cl)cc2)C1)N1C(=O)c2ccccc2C1=O] |

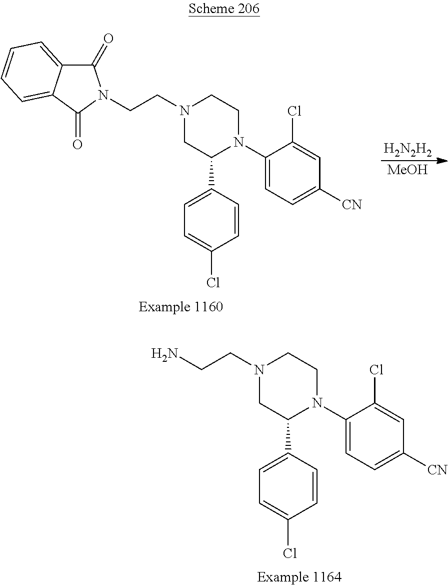 [SMILES: "MeOH" ; N#Cc1ccc(N2CCN(CCN3C(=O)c4ccccc4C3=O)C[C@H]2c2ccc(Cl)cc2)c(Cl)c1 ; "H2N2H2" ; N#Cc1ccc(N2CCN(CCN)C[C@H]2c2ccc(Cl)cc2)c(Cl)c1]

To a solution of Example 1160 (1.4 g, 2.81 mmol) in MeOH was added hydrazine (360 mg, 11.2 mmol). The resultant solution was heated to reflux with stirring for 3 hours. After the reaction was determined to be complete, the solution was concentrated in vacuo. To the residue was added EtOAc, the solids were removed via filtration, and the solvent was removed in vacuo. The crude product was purified via flash chromatography [SiO₂: gradient elution 100:0:0:0 96:4:0.2:0.2 CH₂Cl₂:MeOH:7 N NH₃ (in MeOH): conc NH₄OH (aq.)] to afford Example 1164 (ca. 700 mg).

The following phthalimides were converted to the amines following a procedure similar to that described in Scheme 206.

TABLE L

| Ex. | Phthalamide | Ex. | Amine |
|---|---|---|---|
| 1161 | 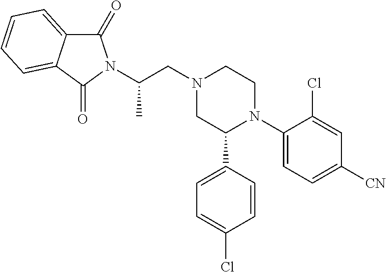 [SMILES: C[C@@H](CN1CCN(c2ccc(C#N)cc2Cl)[C@H](c2ccc(Cl)cc2)C1)N1C(=O)c2ccccc2C1=O] | 1165 | 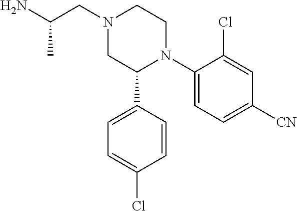 [SMILES: C[C@H](N)CN1CCN(c2ccc(C#N)cc2Cl)[C@H](c2ccc(Cl)cc2)C1] |
| 1162 | 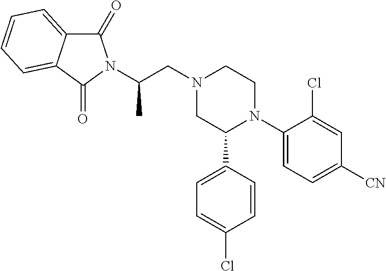 [SMILES: C[C@H](CN1CCN(c2ccc(C#N)cc2Cl)[C@H](c2ccc(Cl)cc2)C1)N1C(=O)c2ccccc2C1=O] | 1166 | 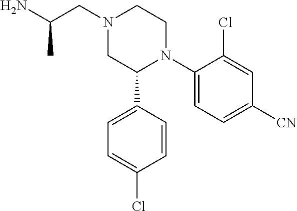 [SMILES: C[C@@H](N)CN1CCN(c2ccc(C#N)cc2Cl)[C@H](c2ccc(Cl)cc2)C1] |

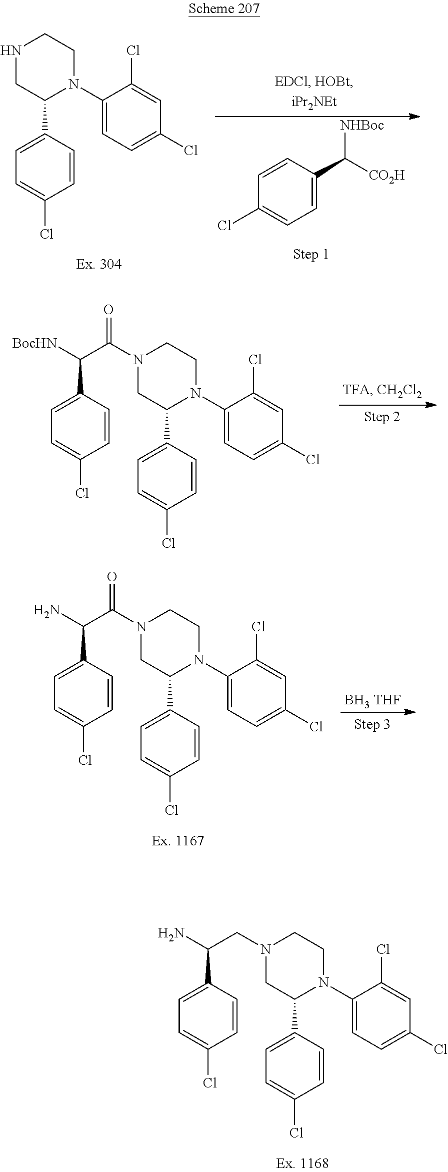 [SMILES: Clc1ccc([C@@H]2CNCCN2c2ccc(Cl)cc2Cl)cc1 ; CC(C)(C)OC(=O)N[C@@H](C(=O)O)c1ccc(Cl)cc1 ; CC(C)(C)OC(=O)N[C@@H](C(=O)N1CCN(c2ccc(Cl)cc2Cl)[C@H](c2ccc(Cl)cc2)C1)c1ccc(Cl)cc1 ; N[C@@H](C(=O)N1CCN(c2ccc(Cl)cc2Cl)[C@H](c2ccc(Cl)cc2)C1)c1ccc(Cl)cc1 ; "BH3 THF" ; N[C@@H](CN1CCN(c2ccc(Cl)cc2Cl)[C@H](c2ccc(Cl)cc2)C1)c1ccc(Cl)cc1]

### Step 1:

To a solution of Ex. 304 (300 mg, 0.88 mmol) in MeCN (1.5 mL) was added EDCI (253 mg, 1.32 mmol), HOBt (178 mg, 1.32 mmol), iPr₂NEt (122 mg, 0.96 mmol) and the acid from step 1 (300 mg, 1.1 mmol). The solution was stirred at RT for 48 h. After that time, the solution was concentrated and purified via flash chromatography (SiO₂: gradient elution 100:0 to 85:15 hexanes:EtOAc) to afford the amide (600 mg).

### Step 2:

To a solution of the amide from step 1 (600 mg) in CH₂Cl₂ (10 mL) was added TFA. The solution was stirred at RT for 1 h. After that time, the solution was basified by the addition of excess sat. Na₂CO₃ (aq.). The mixture was extracted with CH₂Cl₂. The organic layer was dried over Na₂SO₄, filtered and concentrated to afford Ex. 1167.

### Step 3:

To a solution of Ex. 1167 in anhydrous THF (10 mL) was added borane.THF complex (1 M in THF, 3 mL, 3 mmol). The resultant solution was heated to reflux with stirring for 3 h. After the reaction was complete, the solution was cooled to RT and excess hydrochloric acid was added (6 N). The mixture was stirred for 30 min. The mixture was then basified by the addition of excess sat NaHCO₃ (aq.) and the mixture was extracted with EtOAc (3×). The combined organic layers were dried over Na₂SO₄, filtered and concentrated to afford Ex. 1168 that was used without further purification.

The following amines were prepared using a similar procedure to that described in Scheme 207.

TABLE LI

| | Carboxylic | | Amino | | Piperazine |
|---|---|---|---|---|---|
| Piperazine | acid | Ex. | Amide | Ex | Amine |
| 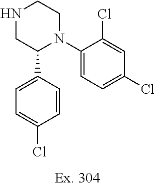 [SMILES: Clc1ccc([C@@H]2CNCCN2c2ccc(Cl)cc2Cl)cc1] | 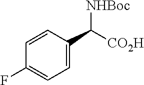 [SMILES: CC(C)(C)OC(=O)N[C@@H](C(=O)O)c1ccc(F)cc1] | 1169 | 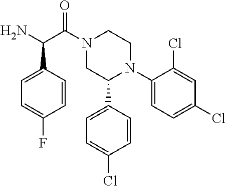 [SMILES: N[C@@H](C(=O)N1CCN(c2ccc(Cl)cc2Cl)[C@H](c2ccc(Cl)cc2)C1)c1ccc(F)cc1] | 1170 | 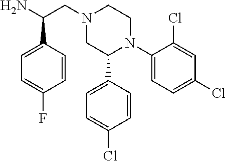 [SMILES: N[C@@H](CN1CCN(c2ccc(Cl)cc2Cl)[C@H](c2ccc(Cl)cc2)C1)c1ccc(F)cc1] |
| 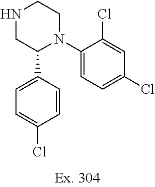 [SMILES: Clc1ccc([C@@H]2CNCCN2c2ccc(Cl)cc2Cl)cc1] | 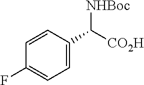 [SMILES: CC(C)(C)OC(=O)N[C@H](C(=O)O)c1ccc(F)cc1] | 1171 | 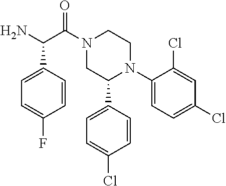 [SMILES: N[C@H](C(=O)N1CCN(c2ccc(Cl)cc2Cl)[C@H](c2ccc(Cl)cc2)C1)c1ccc(F)cc1] | 1172 | 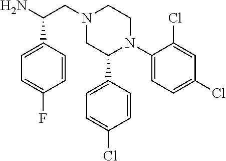 [SMILES: N[C@H](CN1CCN(c2ccc(Cl)cc2Cl)[C@H](c2ccc(Cl)cc2)C1)c1ccc(F)cc1] |

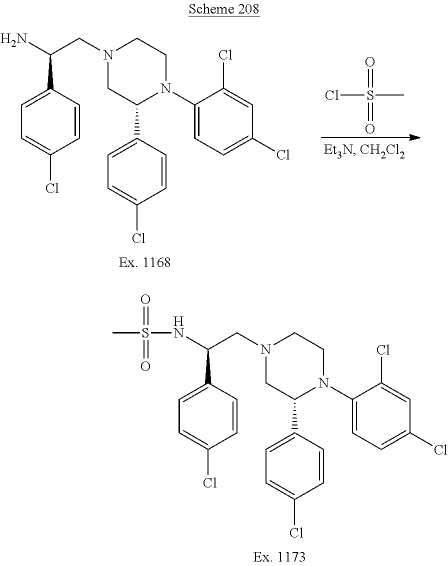

To a solution of Example 1168 (50 mg, 0.1 mmol) in CH₂Cl₂ (2 mL) was added Et₃N (70 μL, 0.5 mmol) and methanesulfonyl chloride (17 mg, 0.15 mmol). The resultant solution was stirred at RT overnight. The solution was concentrated and the crude residue was purified via preparative TLC [SiO₂: 97:3:0.3 CH₂Cl₂: MeOH: conc. NH₄OH (aq.)] to afford Example 1173 (33 mg).

The following amines were prepared using a similar procedure to that described in Scheme 208.

TABLE LII

| | Sulfonyl | | |
|---|---|---|---|
| | chloride | | |
| | or | | Piperazine |
| Amine | anhydride | Ex. | Amine |
| 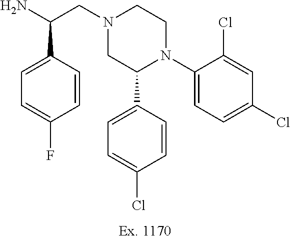 [SMILES: N[C@@H](CN1CCN(c2ccc(Cl)cc2Cl)[C@H](c2ccc(Cl)cc2)C1)c1ccc(F)cc1] | 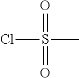 | 1174 | 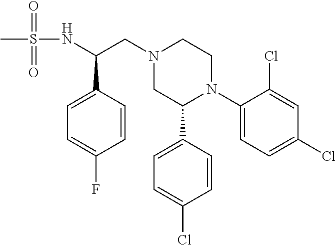 [SMILES: CS(=O)(=O)N[C@@H](CN1CCN(c2ccc(Cl)cc2Cl)[C@H](c2ccc(Cl)cc2)C1)c1ccc(F)cc1] |
| 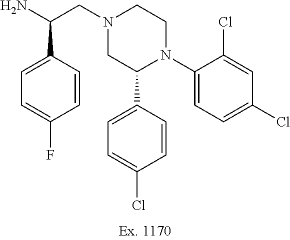 [SMILES: N[C@@H](CN1CCN(c2ccc(Cl)cc2Cl)[C@H](c2ccc(Cl)cc2)C1)c1ccc(F)cc1] | 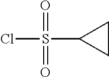 | 1175 | 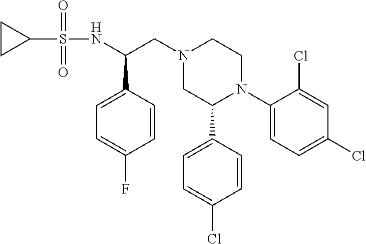 [SMILES: O=S(=O)(N[C@@H](CN1CCN(c2ccc(Cl)cc2Cl)[C@H](c2ccc(Cl)cc2)C1)c1ccc(F)cc1)C1CC1] |
| 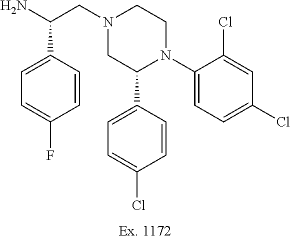 [SMILES: N[C@H](CN1CCN(c2ccc(Cl)cc2Cl)[C@H](c2ccc(Cl)cc2)C1)c1ccc(F)cc1] | 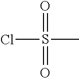 | 1176 | 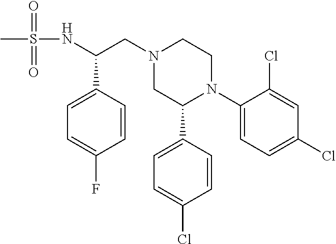 [SMILES: CS(=O)(=O)N[C@H](CN1CCN(c2ccc(Cl)cc2Cl)[C@H](c2ccc(Cl)cc2)C1)c1ccc(F)cc1] |
| 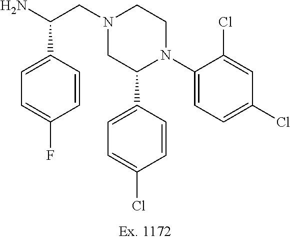 [SMILES: N[C@H](CN1CCN(c2ccc(Cl)cc2Cl)[C@H](c2ccc(Cl)cc2)C1)c1ccc(F)cc1] | 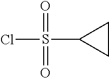 | 1177 | 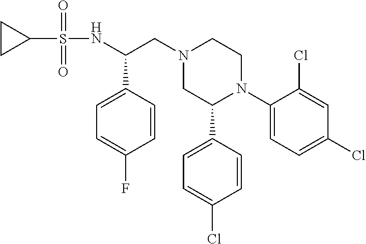 [SMILES: O=S(=O)(N[C@H](CN1CCN(c2ccc(Cl)cc2Cl)[C@H](c2ccc(Cl)cc2)C1)c1ccc(F)cc1)C1CC1] |
| 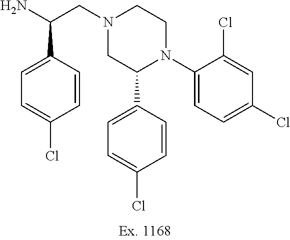 [SMILES: N[C@@H](CN1CCN(c2ccc(Cl)cc2Cl)[C@H](c2ccc(Cl)cc2)C1)c1ccc(Cl)cc1] | 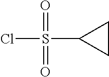 | 1178 | 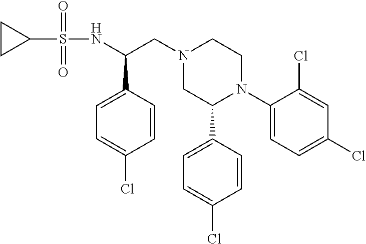 [SMILES: O=S(=O)(N[C@@H](CN1CCN(c2ccc(Cl)cc2Cl)[C@H](c2ccc(Cl)cc2)C1)c1ccc(Cl)cc1)C1CC1] |
| 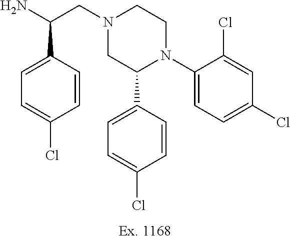 [SMILES: N[C@@H](CN1CCN(c2ccc(Cl)cc2Cl)[C@H](c2ccc(Cl)cc2)C1)c1ccc(Cl)cc1] | AC₂O | 1179 | 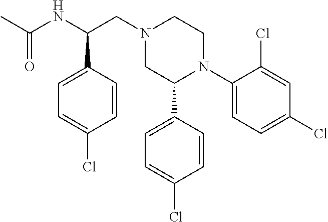 [SMILES: CC(=O)N[C@@H](CN1CCN(c2ccc(Cl)cc2Cl)[C@H](c2ccc(Cl)cc2)C1)c1ccc(Cl)cc1] |

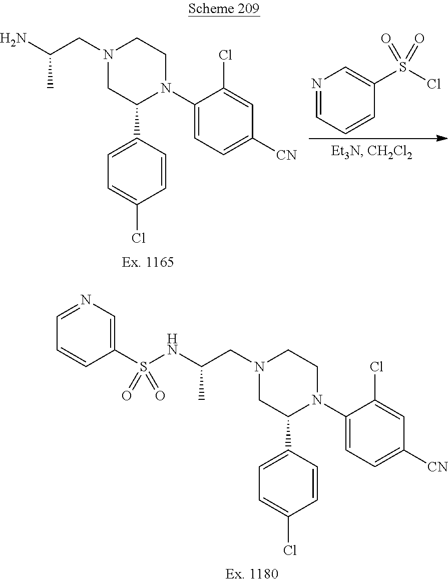 [SMILES: C[C@H](N)CN1CCN(c2ccc(C#N)cc2Cl)[C@H](c2ccc(Cl)cc2)C1 ; O=S(=O)(Cl)c1cccnc1 ; C[C@@H](CN1CCN(c2ccc(C#N)cc2Cl)[C@H](c2ccc(Cl)cc2)C1)NS(=O)(=O)c1cccnc1]

To a solution of Example 1165 (50 mg, 0.10 mmol) in CH₂Cl₂ (10 mL) was added Et₃N (15 mg, 0.15 mmol) and 3-pyridinesulfonyl chloride (21 mg, 0.12 mmol). The resultant solution was stirred at RT overnight. The solution was concentrated. The crude residue was purified via flash chromatography (SiO₂: gradient elution 100:0:0 to 96.6:3.5:0.35 CH₂Cl₂:MeOH:conc. NH₄OH_{(aq.)}) to afford Example 1180 (30 mg).

The following sulfonamides were prepared using a method similar to that described in Scheme 209.

TABLE LIII

| | | Sulfonyl | | |
|---|---|---|---|---|
| Ex | | chloride | Ex | |
| 1164 | 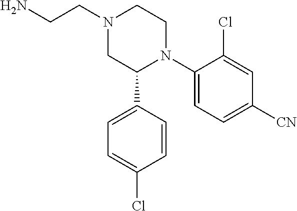 [SMILES: N#Cc1ccc(N2CCN(CCN)C[C@H]2c2ccc(Cl)cc2)c(Cl)c1] | 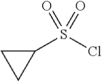 | 1181 | 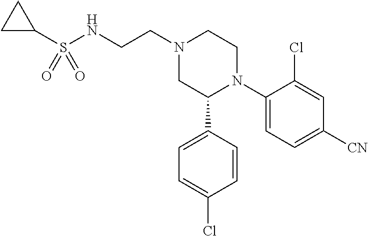 [SMILES: N#Cc1ccc(N2CCN(CCNS(=O)(=O)C3CC3)C[C@H]2c2ccc(Cl)cc2)c(Cl)c1] |
| 1164 | 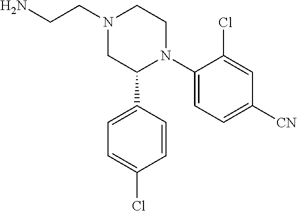 [SMILES: N#Cc1ccc(N2CCN(CCN)C[C@H]2c2ccc(Cl)cc2)c(Cl)c1] | 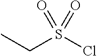 | 1182 | 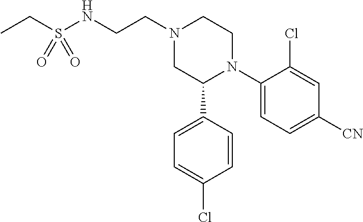 [SMILES: CCS(=O)(=O)NCCN1CCN(c2ccc(C#N)cc2Cl)[C@H](c2ccc(Cl)cc2)C1] |
| 1164 | 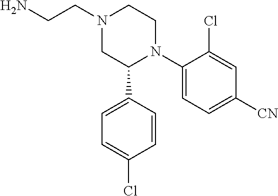 [SMILES: N#Cc1ccc(N2CCN(CCN)C[C@H]2c2ccc(Cl)cc2)c(Cl)c1] | 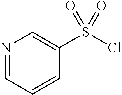 | 1183 | 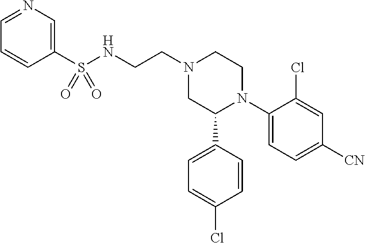 [SMILES: N#Cc1ccc(N2CCN(CCNS(=O)(=O)c3cccnc3)C[C@H]2c2ccc(Cl)cc2)c(Cl)c1] |
| 1165 | 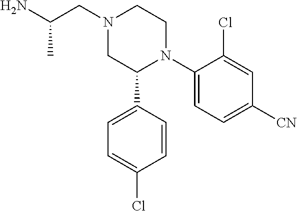 [SMILES: C[C@H](N)CN1CCN(c2ccc(C#N)cc2Cl)[C@H](c2ccc(Cl)cc2)C1] | 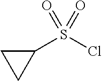 | 1184 | 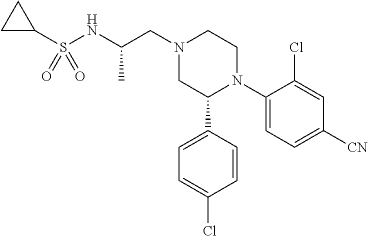 [SMILES: C[C@@H](CN1CCN(c2ccc(C#N)cc2Cl)[C@H](c2ccc(Cl)cc2)C1)NS(=O)(=O)C1CC1] |
| 1165 | 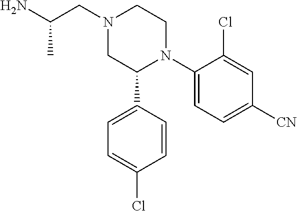 [SMILES: C[C@H](N)CN1CCN(c2ccc(C#N)cc2Cl)[C@H](c2ccc(Cl)cc2)C1] | 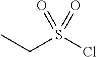 | 1185 | 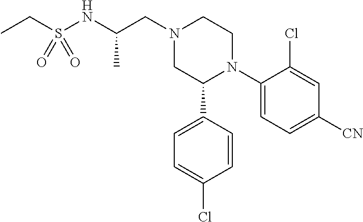 [SMILES: CCS(=O)(=O)N[C@@H](C)CN1CCN(c2ccc(C#N)cc2Cl)[C@H](c2ccc(Cl)cc2)C1] |

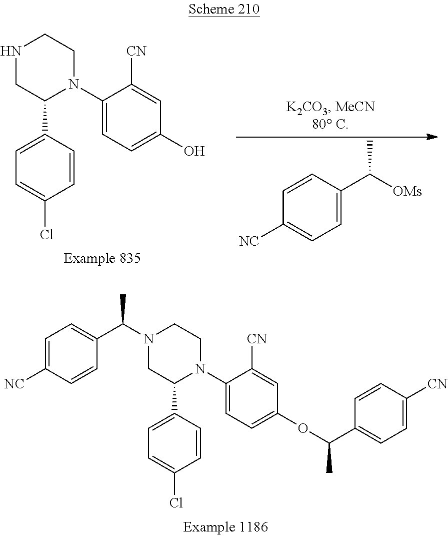 [SMILES: N#Cc1cc(O)ccc1N1CCNC[C@H]1c1ccc(Cl)cc1 ; C[C@H](OS(C)(=O)=O)c1ccc(C#N)cc1 ; C[C@H](c1ccc(C#N)cc1)N1CCN(c2ccc(O[C@H](C)c3ccc(C#N)cc3)cc2C#N)[C@H](c2ccc(Cl)cc2)C1]

To a solution of Example 835 (30 mg) in MeCN (5 mL) was added the mesylate from Scheme 66 (30 mg) followed by K₂CO₃ (100 mg). The resulting solution was heated to 80° C. overnight. The mixture was concentrated and purified via prep TLC (SiO₂: 1:1 EtOAc:hexanes) to afford Example 1186.

### LC/MS Conditions

The following conditions were used to determine the retention times and mass of final compounds listed in Table LIV

Column: Gemini C-18, 50×4.6 mm, 5 micron, obtained from Phenomenex.

Mobile phase: A: 0.05% Trifluoacetic acid in water B: 0.05% Trifluoacetic acid in acetonitrile

Gradient: 90% A and 10% B to 5% A and 95% B in 5 minutes

Flow rate: 1.0 mL/min

UV detection: 254 nm
ESI-MS: Electro Spray Ionization Liquid chromatography-mass spectrometry (ESI-LC/MS) was performed on a PE SCIEX API-150EX, single quadrupole mass spectrometer.

The following compounds of Formula (I) shown in Table LIV (which are compounds of the invention) were prepared using the synthetic methods described above or analogous thereto. Pharmaceutically acceptable salts of the compounds in Table LIV, including each of the salts described herein, and solvates, esters, prodrugs, tautomers, and stereoisomers of such compounds and salts thereof are also within the scope of the present invention.

All of the compounds in Table LIV exhibited Ki values in the assay described above from about 0.2 nM to about 2 uM. All of the compounds of Table LIV below, except those of examples 826e, 807, 813b, 817, 822, 824, 826b, 826d, 830, 831, 877b, and 1167a exhibited Ki values of less than 900 nM. Some of the compounds of Table LIV, including those of examples 820b and 873, exhibited Ki values of from about 100 nM to about 1 uM. Some of the compounds of Table LIV, including those of examples 930, 949, 1035 g, 1037, and 1091, exhibited Ki values of from about 0.2 nM to about 100 nM. The compound of example 1089 exhibited a Ki value of about 873 nM.

TABLE LIV

| | | [M + H] | retent. |
|---|---|---|---|
| Example | Structure | obsvd | time (min.) |
| 803 | 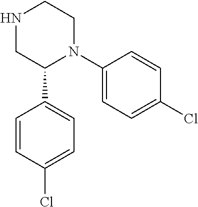 [SMILES: Clc1ccc([C@@H]2CNCCN2c2ccc(Cl)cc2)cc1] | 3.07 | 3.18 |
| 805 | 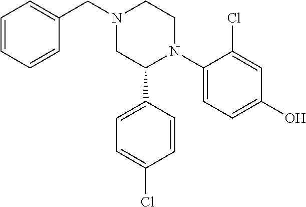 [SMILES: Oc1ccc(N2CCN(Cc3ccccc3)C[C@H]2c2ccc(Cl)cc2)c(Cl)c1] | 413.2 | 3.50 |
| 806 | 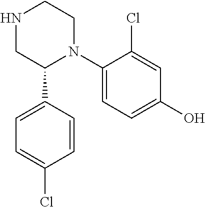 [SMILES: Oc1ccc(N2CCNC[C@H]2c2ccc(Cl)cc2)c(Cl)c1] | 323.2 | 2.81 |
| 807 | 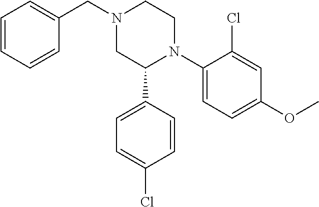 [SMILES: COc1ccc(N2CCN(Cc3ccccc3)C[C@H]2c2ccc(Cl)cc2)c(Cl)c1] | 427.2 | 3.81 |
| 808 | 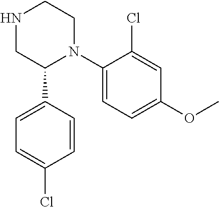 [SMILES: COc1ccc(N2CCNC[C@H]2c2ccc(Cl)cc2)c(Cl)c1] | 337.2 | 3.33 |
| 809 | 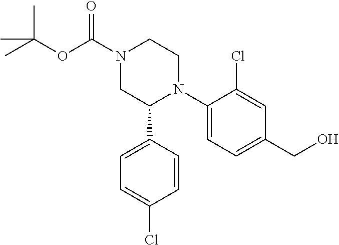 [SMILES: CC(C)(C)OC(=O)N1CCN(c2ccc(CO)cc2Cl)[C@H](c2ccc(Cl)cc2)C1] | 437.2 | 5.27 |
| 811 | 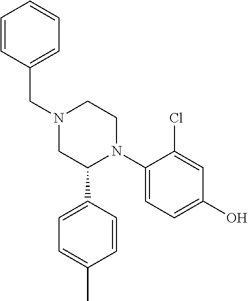 [SMILES: Cc1ccc([C@@H]2CN(Cc3ccccc3)CCN2c2ccc(O)cc2Cl)cc1] | 393.2 | 3.39 |
| 812b | 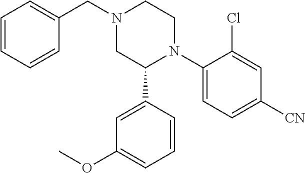 [SMILES: COc1cccc([C@@H]2CN(Cc3ccccc3)CCN2c2ccc(C#N)cc2Cl)c1] | 418.2 | 3.42 |
| 812 | 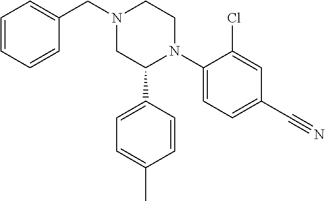 [SMILES: Cc1ccc([C@@H]2CN(Cc3ccccc3)CCN2c2ccc(C#N)cc2Cl)cc1] | 402.2 | 3.22 |
| 813b | 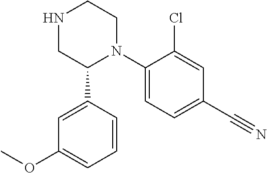 [SMILES: COc1cccc([C@@H]2CNCCN2c2ccc(C#N)cc2Cl)c1] | 328.2 | 2.67 |
| 813a | 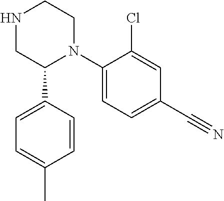 [SMILES: Cc1ccc([C@@H]2CNCCN2c2ccc(C#N)cc2Cl)cc1] | 312.2 | 3.03 |
| 814 | 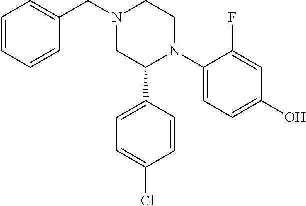 [SMILES: Oc1ccc(N2CCN(Cc3ccccc3)C[C@H]2c2ccc(Cl)cc2)c(F)c1] | 397.2 | 3.21 |
| 815 | 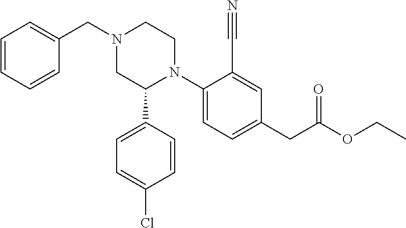 [SMILES: CCOC(=O)Cc1ccc(N2CCN(Cc3ccccc3)C[C@H]2c2ccc(Cl)cc2)c(C#N)c1] | 474.3 | 3.76 |
| 816 | 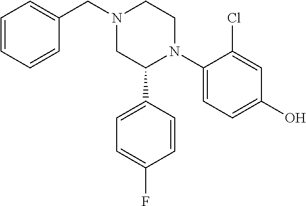 [SMILES: Oc1ccc(N2CCN(Cc3ccccc3)C[C@H]2c2ccc(F)cc2)c(Cl)c1] | 397.2 | 3.28 |
| 817 | 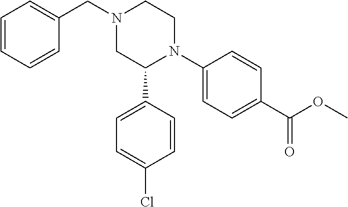 [SMILES: COC(=O)c1ccc(N2CCN(Cc3ccccc3)C[C@H]2c2ccc(Cl)cc2)cc1] | 421.2 | 3.47 |
| 818 | 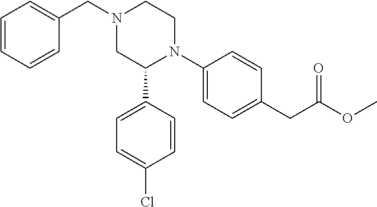 [SMILES: COC(=O)Cc1ccc(N2CCN(Cc3ccccc3)C[C@H]2c2ccc(Cl)cc2)cc1] | 435.2 | 3.54 |
| 819 | 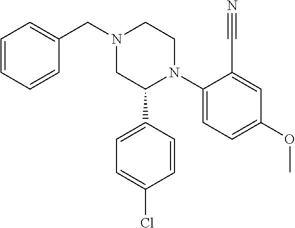 [SMILES: COc1ccc(N2CCN(Cc3ccccc3)C[C@H]2c2ccc(Cl)cc2)c(C#N)c1] | 418.2 | 3.26 |
| 820 | 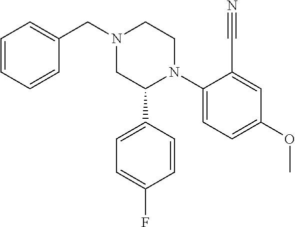 [SMILES: COc1ccc(N2CCN(Cc3ccccc3)C[C@H]2c2ccc(F)cc2)c(C#N)c1] | 402.2 | 3.18 |
| 820b | 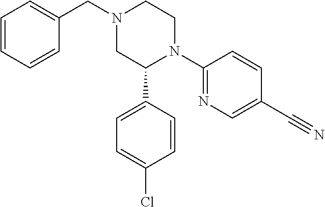 [SMILES: N#Cc1ccc(N2CCN(Cc3ccccc3)C[C@H]2c2ccc(Cl)cc2)nc1] | 389.2 | 3.15 |
| 820d | 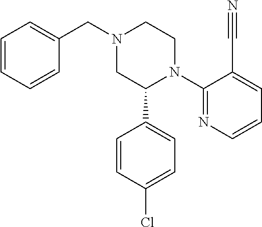 [SMILES: N#Cc1cccnc1N1CCN(Cc2ccccc2)C[C@H]1c1ccc(Cl)cc1] | 389.2 | 3.37 |
| 820e | 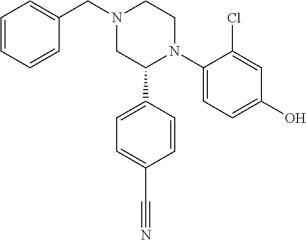 [SMILES: N#Cc1ccc([C@@H]2CN(Cc3ccccc3)CCN2c2ccc(O)cc2Cl)cc1] | 404.2 | 2.84 |
| 821 | 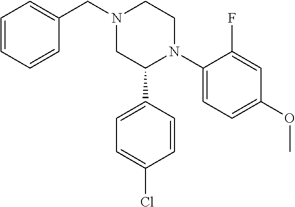 [SMILES: COc1ccc(N2CCN(Cc3ccccc3)C[C@H]2c2ccc(Cl)cc2)c(F)c1] | 411.2 | 3.34 |
| 822 | 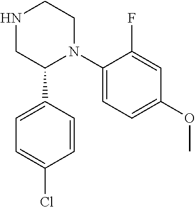 [SMILES: COc1ccc(N2CCNC[C@H]2c2ccc(Cl)cc2)c(F)c1] | 321.2 | 2.89 |
| 823 | 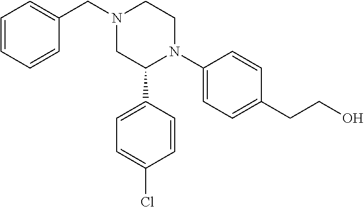 [SMILES: OCCc1ccc(N2CCN(Cc3ccccc3)C[C@H]2c2ccc(Cl)cc2)cc1] | 407.2 | 3.13 |
| 824 | 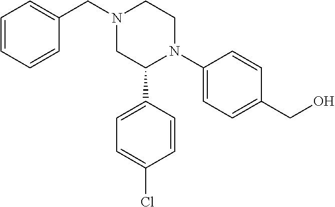 [SMILES: OCc1ccc(N2CCN(Cc3ccccc3)C[C@H]2c2ccc(Cl)cc2)cc1] | 393.2 | 3.06 |
| 826 | 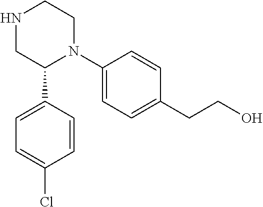 [SMILES: OCCc1ccc(N2CCNC[C@H]2c2ccc(Cl)cc2)cc1] | 317.2 | 2.50 |
| 826b | 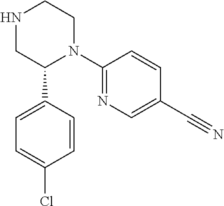 [SMILES: N#Cc1ccc(N2CCNC[C@H]2c2ccc(Cl)cc2)nc1] | 299.2 | 2.59 |
| 826a | 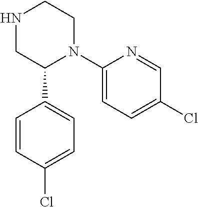 [SMILES: Clc1ccc([C@@H]2CNCCN2c2ccc(Cl)cn2)cc1] | 308.2 | 2.65 |
| 826d | 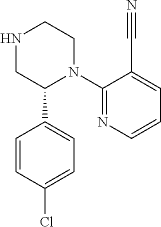 [SMILES: N#Cc1cccnc1N1CCNC[C@H]1c1ccc(Cl)cc1] | 299.2 | 2.68 |
| 826e | 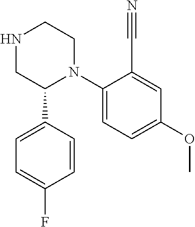 [SMILES: COc1ccc(N2CCNC[C@H]2c2ccc(F)cc2)c(C#N)c1] | 317.2 | 2.84 |
| 827 | 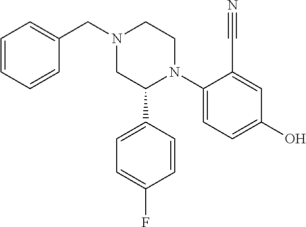 [SMILES: N#Cc1cc(O)ccc1N1CCN(Cc2ccccc2)C[C@H]1c1ccc(F)cc1] | 388.2 | 3.07 |
| 828 | 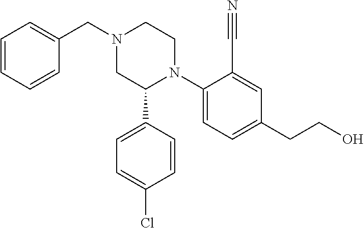 [SMILES: N#Cc1cc(CCO)ccc1N1CCN(Cc2ccccc2)C[C@H]1c1ccc(Cl)cc1] | 432.2 | 2.90 |
| 829 | 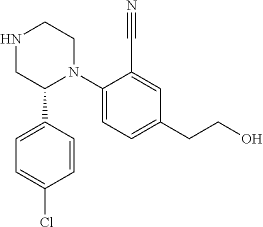 [SMILES: N#Cc1cc(CCO)ccc1N1CCNC[C@H]1c1ccc(Cl)cc1] | 342.2 | 2.37 |
| 830 | 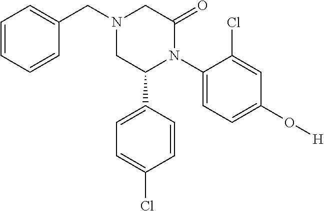 [SMILES: O=C1CN(Cc2ccccc2)C[C@@H](c2ccc(Cl)cc2)N1c1ccc(O)cc1Cl] | 427.2 | 3.64 |
| 831 | 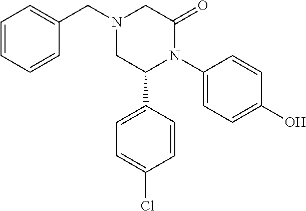 [SMILES: O=C1CN(Cc2ccccc2)C[C@@H](c2ccc(Cl)cc2)N1c1ccc(O)cc1] | 393.2 | 3.14 |
| 832 | 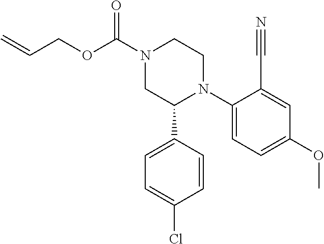 [SMILES: C=CCOC(=O)N1CCN(c2ccc(OC)cc2C#N)[C@H](c2ccc(Cl)cc2)C1] | 412.2 | 4.96 |
| 833 | 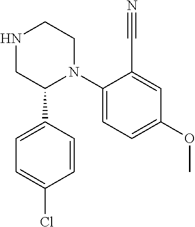 [SMILES: COc1ccc(N2CCNC[C@H]2c2ccc(Cl)cc2)c(C#N)c1] | 328.2 | 2.85 |
| 834 | 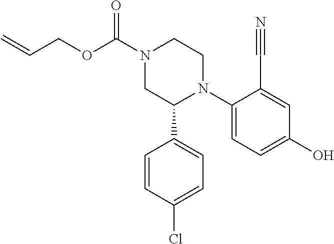 [SMILES: C=CCOC(=O)N1CCN(c2ccc(O)cc2C#N)[C@H](c2ccc(Cl)cc2)C1] | 398.2 | 4.89 |
| 835 | 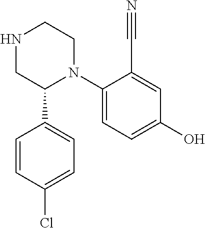 [SMILES: N#Cc1cc(O)ccc1N1CCNC[C@H]1c1ccc(Cl)cc1] | 314.2 | 2.45 |
| 836 | 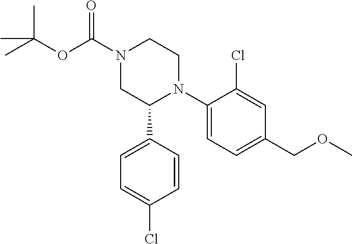 [SMILES: COCc1ccc(N2CCN(C(=O)OC(C)(C)C)C[C@H]2c2ccc(Cl)cc2)c(Cl)c1] | 451.2 | 5.98 |
| 837 | 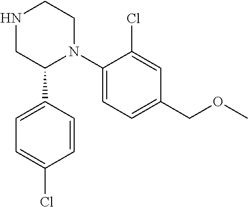 [SMILES: COCc1ccc(N2CCNC[C@H]2c2ccc(Cl)cc2)c(Cl)c1] | 351.2 | 3.28 |
| 839 | 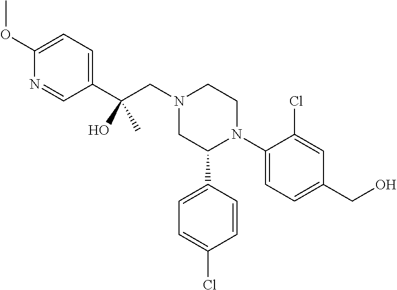 [SMILES: COc1ccc([C@](C)(O)CN2CCN(c3ccc(CO)cc3Cl)[C@H](c3ccc(Cl)cc3)C2)cn1] | 502.3 | 3.06 |
| 840 | 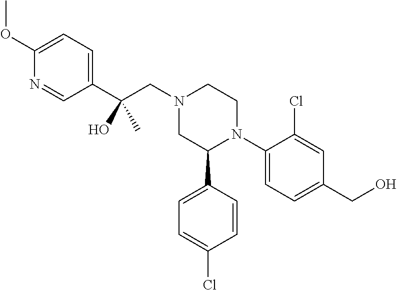 [SMILES: COc1ccc([C@](C)(O)CN2CCN(c3ccc(CO)cc3Cl)[C@@H](c3ccc(Cl)cc3)C2)cn1] | 502.3 | 3.04 |
| 841 | 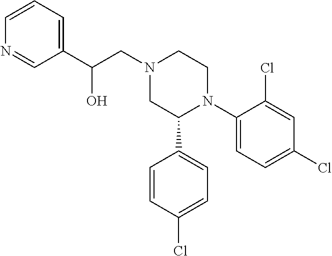 [SMILES: OC(CN1CCN(c2ccc(Cl)cc2Cl)[C@H](c2ccc(Cl)cc2)C1)c1cccnc1] | 462.3 | 2.85 |
| 842 | 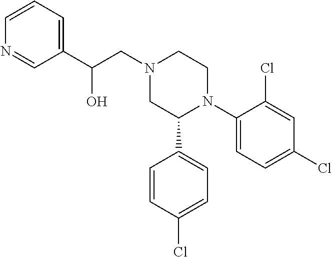 [SMILES: OC(CN1CCN(c2ccc(Cl)cc2Cl)[C@H](c2ccc(Cl)cc2)C1)c1cccnc1] | 462.3 | 3.08 |
| 842c | 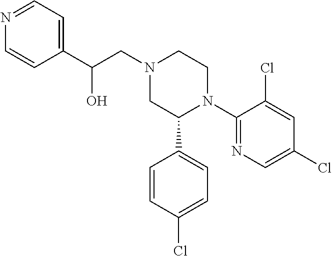 [SMILES: OC(CN1CCN(c2ncc(Cl)cc2Cl)[C@H](c2ccc(Cl)cc2)C1)c1ccncc1] | 465.3 | 2.80 |
| 844 | 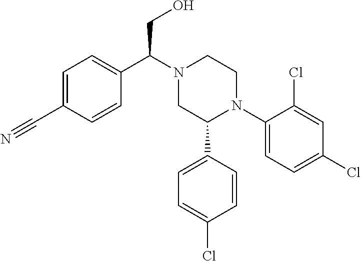 [SMILES: N#Cc1ccc([C@@H](CO)N2CCN(c3ccc(Cl)cc3Cl)[C@H](c3ccc(Cl)cc3)C2)cc1] | 486.3 | 3.63 |
| 845 | 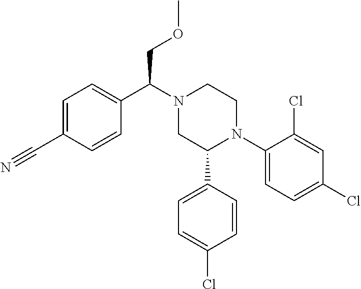 [SMILES: COC[C@H](c1ccc(C#N)cc1)N1CCN(c2ccc(Cl)cc2Cl)[C@H](c2ccc(Cl)cc2)C1] | 500.3 | 4.08 |
| 846 | 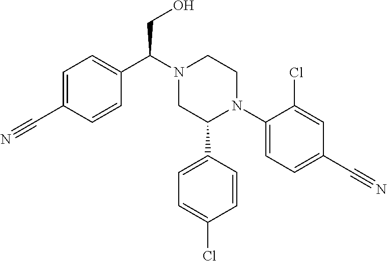 [SMILES: N#Cc1ccc([C@@H](CO)N2CCN(c3ccc(C#N)cc3Cl)[C@H](c3ccc(Cl)cc3)C2)cc1] | 477.3 | 3.36 |
| 847a | 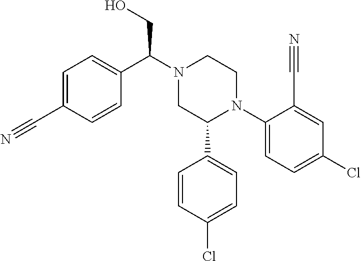 [SMILES: N#Cc1ccc([C@@H](CO)N2CCN(c3ccc(Cl)cc3C#N)[C@H](c3ccc(Cl)cc3)C2)cc1] | 477.3 | 3.58 |
| 847 | 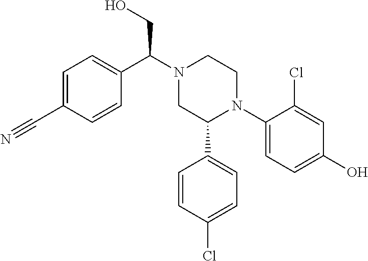 [SMILES: N#Cc1ccc([C@@H](CO)N2CCN(c3ccc(O)cc3Cl)[C@H](c3ccc(Cl)cc3)C2)cc1] | 468.3 | 3.11 |
| 848 | 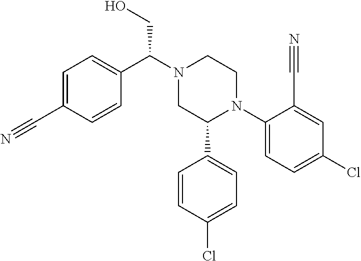 [SMILES: N#Cc1ccc([C@H](CO)N2CCN(c3ccc(Cl)cc3C#N)[C@H](c3ccc(Cl)cc3)C2)cc1] | 477.3 | 3.60 |
| 850 | 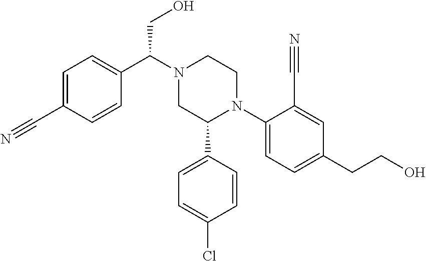 [SMILES: N#Cc1ccc([C@H](CO)N2CCN(c3ccc(CCO)cc3C#N)[C@H](c3ccc(Cl)cc3)C2)cc1] | 487.3 | 2.96 |
| 851 | 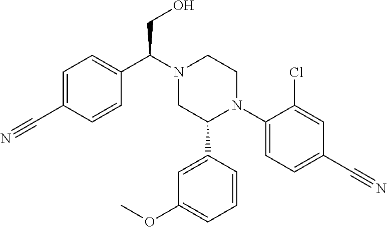 [SMILES: COc1cccc([C@@H]2CN([C@H](CO)c3ccc(C#N)cc3)CCN2c2ccc(C#N)cc2Cl)c1] | 473.3 | 3.23 |
| 852 | 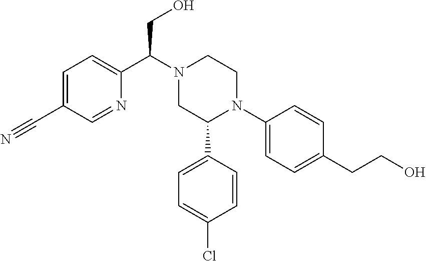 [SMILES: N#Cc1ccc([C@@H](CO)N2CCN(c3ccc(CCO)cc3)[C@H](c3ccc(Cl)cc3)C2)nc1] | 463.3 | 2.79 |
| 853 | 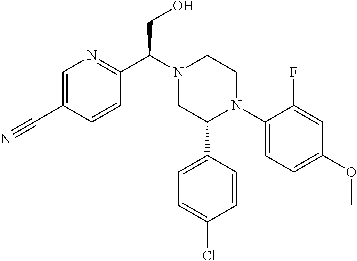 [SMILES: COc1ccc(N2CCN([C@H](CO)c3ccc(C#N)cn3)C[C@H]2c2ccc(Cl)cc2)c(F)c1] | 467.3 | 3.32 |
| 854 | 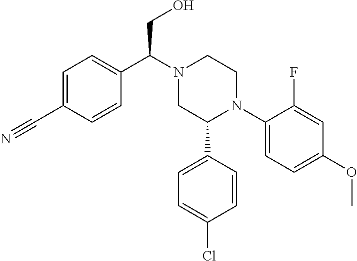 [SMILES: COc1ccc(N2CCN([C@H](CO)c3ccc(C#N)cc3)C[C@H]2c2ccc(Cl)cc2)c(F)c1] | 466.3 | 3.17 |
| 855 | 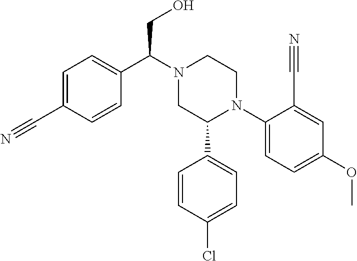 [SMILES: COc1ccc(N2CCN([C@H](CO)c3ccc(C#N)cc3)C[C@H]2c2ccc(Cl)cc2)c(C#N)c1] | 473.3 | 3.35 |
| 856 | 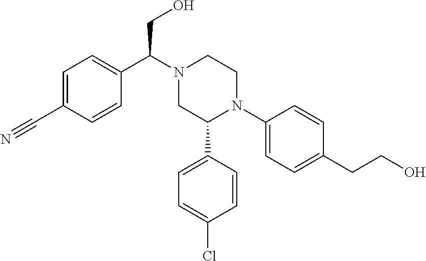 [SMILES: N#Cc1ccc([C@@H](CO)N2CCN(c3ccc(CCO)cc3)[C@H](c3ccc(Cl)cc3)C2)cc1] | 462.3 | 2.94 |
| 857a | 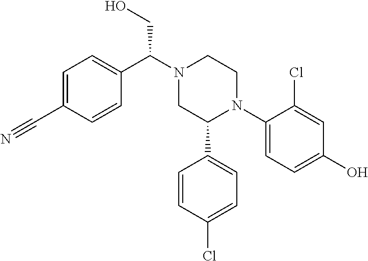 [SMILES: N#Cc1ccc([C@H](CO)N2CCN(c3ccc(O)cc3Cl)[C@H](c3ccc(Cl)cc3)C2)cc1] | 468.3 | 3.25 |
| 857 | 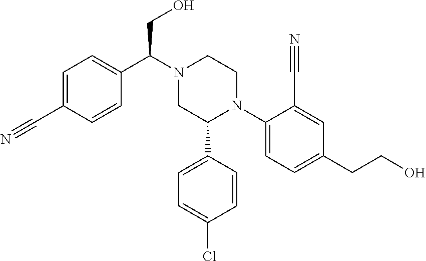 [SMILES: N#Cc1ccc([C@@H](CO)N2CCN(c3ccc(CCO)cc3C#N)[C@H](c3ccc(Cl)cc3)C2)cc1] | 487.3 | 3.02 |
| 858 | 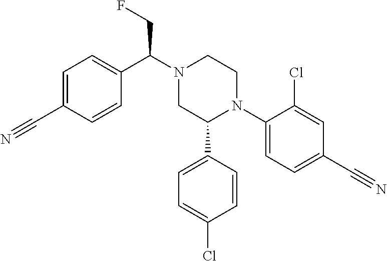 [SMILES: N#Cc1ccc([C@@H](CF)N2CCN(c3ccc(C#N)cc3Cl)[C@H](c3ccc(Cl)cc3)C2)cc1] | 479.3 | 3.66 |
| 859 | 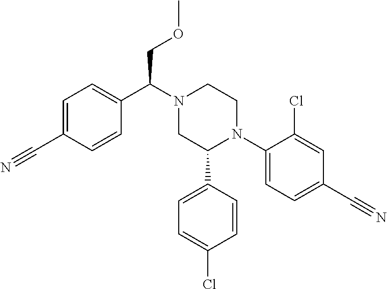 [SMILES: COC[C@H](c1ccc(C#N)cc1)N1CCN(c2ccc(C#N)cc2Cl)[C@H](c2ccc(Cl)cc2)C1] | 491.3 | 3.86 |
| 861 | 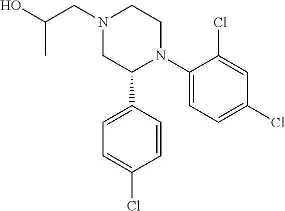 [SMILES: CC(O)CN1CCN(c2ccc(Cl)cc2Cl)[C@H](c2ccc(Cl)cc2)C1] | 399.2 | 3.14 |
| 862 | 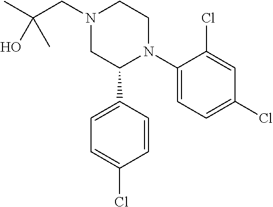 [SMILES: CC(C)(O)CN1CCN(c2ccc(Cl)cc2Cl)[C@H](c2ccc(Cl)cc2)C1] | 413.2 | 3.27 |
| 863 | 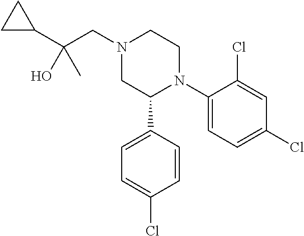 [SMILES: CC(O)(CN1CCN(c2ccc(Cl)cc2Cl)[C@H](c2ccc(Cl)cc2)C1)C1CC1] | 439.2 | 3.51 |
| 864 | 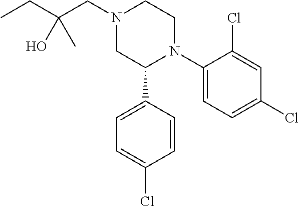 [SMILES: CCC(C)(O)CN1CCN(c2ccc(Cl)cc2Cl)[C@H](c2ccc(Cl)cc2)C1] | 427.2 | 3.37 |
| 865 | 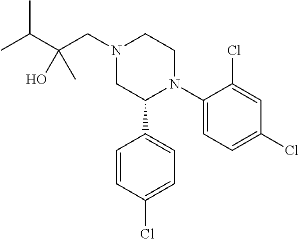 [SMILES: CC(C)C(C)(O)CN1CCN(c2ccc(Cl)cc2Cl)[C@H](c2ccc(Cl)cc2)C1] | 441.3 | 4.05 |
| 866 | 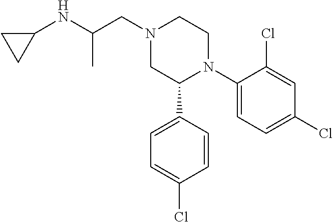 [SMILES: CC(CN1CCN(c2ccc(Cl)cc2Cl)[C@H](c2ccc(Cl)cc2)C1)NC1CC1] | 413.2 | 3.31 |
| 867 | 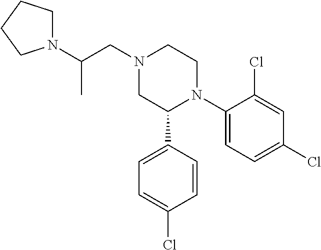 [SMILES: CC(CN1CCN(c2ccc(Cl)cc2Cl)[C@H](c2ccc(Cl)cc2)C1)N1CCCC1] | 452.2 | 3.48 |
| 868c | 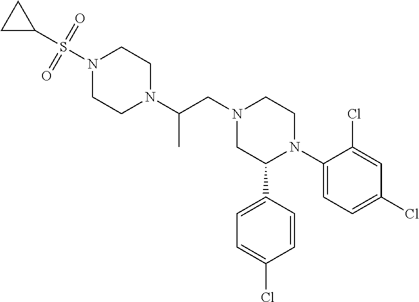 [SMILES: CC(CN1CCN(c2ccc(Cl)cc2Cl)[C@H](c2ccc(Cl)cc2)C1)N1CCN(S(=O)(=O)C2CC2)CC1] | 571.3 | 3.46 |
| 868 | 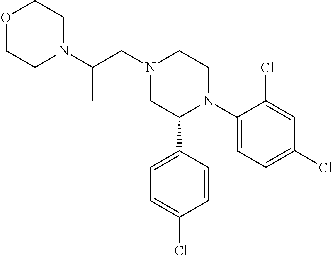 [SMILES: CC(CN1CCN(c2ccc(Cl)cc2Cl)[C@H](c2ccc(Cl)cc2)C1)N1CCOCC1] | 468.3 | 3.35 |
| 870 | 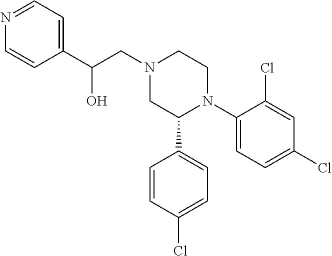 [SMILES: OC(CN1CCN(c2ccc(Cl)cc2Cl)[C@H](c2ccc(Cl)cc2)C1)c1ccncc1] | 462.3 | 3.03 |
| 872 | 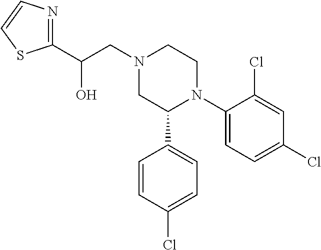 [SMILES: OC(CN1CCN(c2ccc(Cl)cc2Cl)[C@H](c2ccc(Cl)cc2)C1)c1nccs1] | 468.3 | 3.38 |
| 873 | 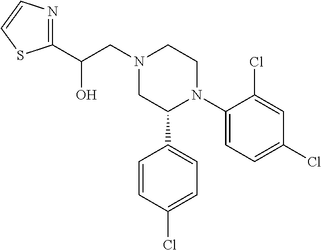 [SMILES: OC(CN1CCN(c2ccc(Cl)cc2Cl)[C@H](c2ccc(Cl)cc2)C1)c1nccs1] | 468.3 | 3.39 |
| 874 | 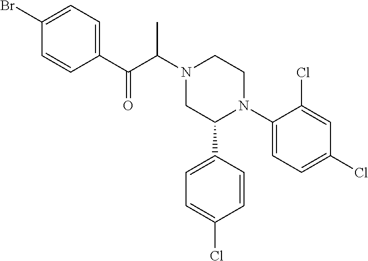 [SMILES: CC(C(=O)c1ccc(Br)cc1)N1CCN(c2ccc(Cl)cc2Cl)[C@H](c2ccc(Cl)cc2)C1] | 553.3 | 4.04 |
| 875 | 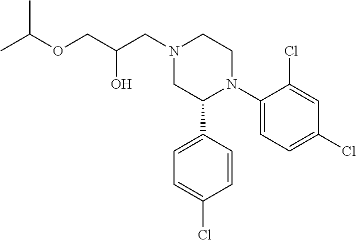 [SMILES: CC(C)OCC(O)CN1CCN(c2ccc(Cl)cc2Cl)[C@H](c2ccc(Cl)cc2)C1] | 457.3 | 3.43 |
| 876 | 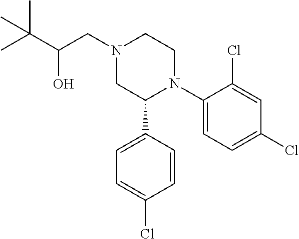 [SMILES: CC(C)(C)C(O)CN1CCN(c2ccc(Cl)cc2Cl)[C@H](c2ccc(Cl)cc2)C1] | 441.2 | 3.56 |
| 877a | 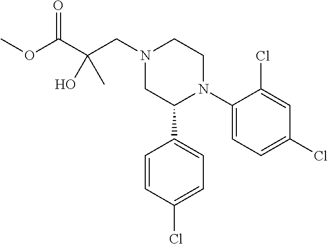 [SMILES: COC(=O)C(C)(O)CN1CCN(c2ccc(Cl)cc2Cl)[C@H](c2ccc(Cl)cc2)C1] | 457.3 | 3.69 |
| 877b | 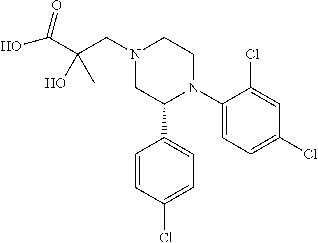 [SMILES: CC(O)(CN1CCN(c2ccc(Cl)cc2Cl)[C@H](c2ccc(Cl)cc2)C1)C(=O)O] | 443.2 | 3.34 |
| 878 | 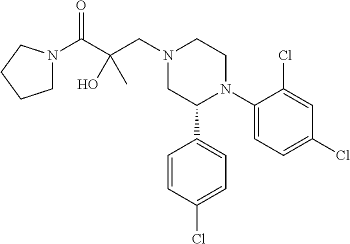 [SMILES: CC(O)(CN1CCN(c2ccc(Cl)cc2Cl)[C@H](c2ccc(Cl)cc2)C1)C(=O)N1CCCC1] | 496.3 | 3.41 |
| 879 | 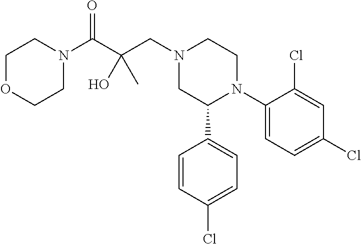 [SMILES: CC(O)(CN1CCN(c2ccc(Cl)cc2Cl)[C@H](c2ccc(Cl)cc2)C1)C(=O)N1CCOCC1] | 512.3 | 3.70 |
| 880 | 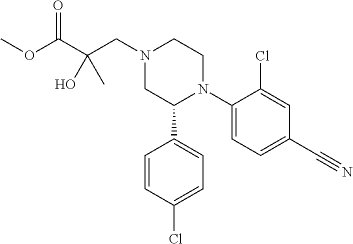 [SMILES: COC(=O)C(C)(O)CN1CCN(c2ccc(C#N)cc2Cl)[C@H](c2ccc(Cl)cc2)C1] | 448.2 | 2.88 |
| 881 | 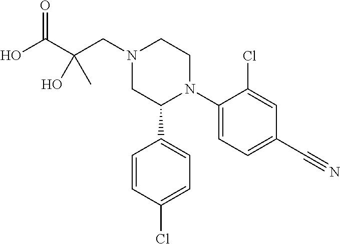 [SMILES: CC(O)(CN1CCN(c2ccc(C#N)cc2Cl)[C@H](c2ccc(Cl)cc2)C1)C(=O)O] | 434.2 | 2.90 |
| 882 | 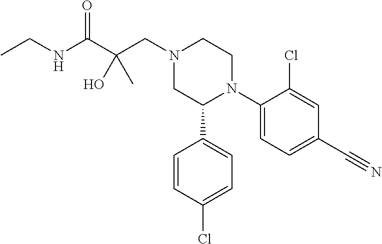 [SMILES: CCNC(=O)C(C)(O)CN1CCN(c2ccc(C#N)cc2Cl)[C@H](c2ccc(Cl)cc2)C1] | 461.3 | 2.94 |
| 883 | 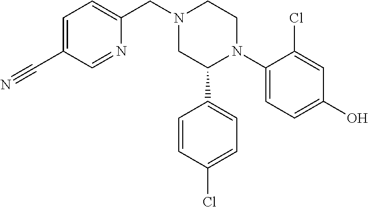 [SMILES: N#Cc1ccc(CN2CCN(c3ccc(O)cc3Cl)[C@H](c3ccc(Cl)cc3)C2)nc1] | 439.2 | 3.20 |
| 884 | 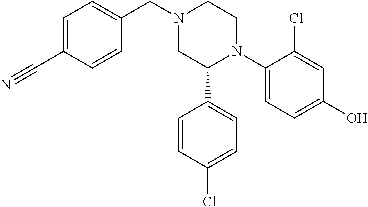 [SMILES: N#Cc1ccc(CN2CCN(c3ccc(O)cc3Cl)[C@H](c3ccc(Cl)cc3)C2)cc1] | 438.2 | 3.06 |
| 885 | 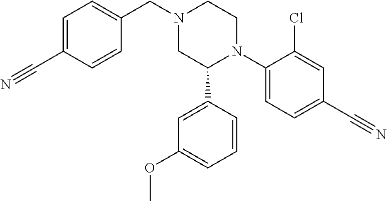 [SMILES: COc1cccc([C@@H]2CN(Cc3ccc(C#N)cc3)CCN2c2ccc(C#N)cc2Cl)c1] | 443.2 | 3.31 |
| 886 | 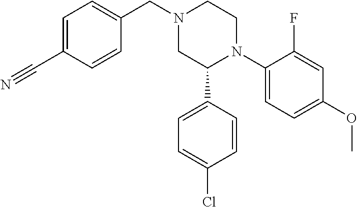 [SMILES: COc1ccc(N2CCN(Cc3ccc(C#N)cc3)C[C@H]2c2ccc(Cl)cc2)c(F)c1] | 436.2 | 3.36 |
| 887a | 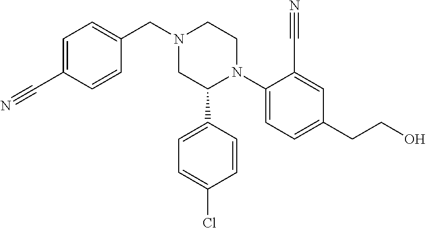 [SMILES: N#Cc1ccc(CN2CCN(c3ccc(CCO)cc3C#N)[C@H](c3ccc(Cl)cc3)C2)cc1] | 457.3 | 3.07 |
| 887b | 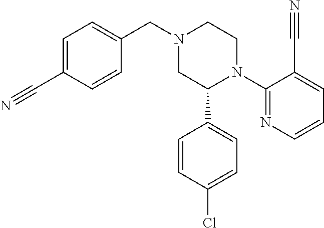 [SMILES: N#Cc1ccc(CN2CCN(c3ncccc3C#N)[C@H](c3ccc(Cl)cc3)C2)cc1] | 414.2 | 3.32 |
| 888 | 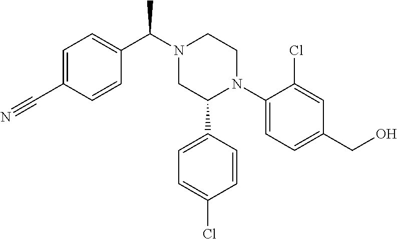 [SMILES: C[C@H](c1ccc(C#N)cc1)N1CCN(c2ccc(CO)cc2Cl)[C@H](c2ccc(Cl)cc2)C1] | 466.3 | 2.96 |
| 889 | 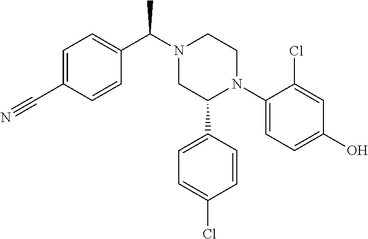 [SMILES: C[C@H](c1ccc(C#N)cc1)N1CCN(c2ccc(O)cc2Cl)[C@H](c2ccc(Cl)cc2)C1] | 452.2 | 3.09 |
| 890 | 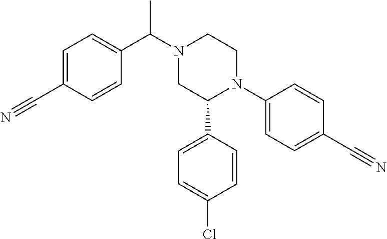 [SMILES: CC(c1ccc(C#N)cc1)N1CCN(c2ccc(C#N)cc2)[C@H](c2ccc(Cl)cc2)C1] | 427.2 | 3.49 |
| 892 | 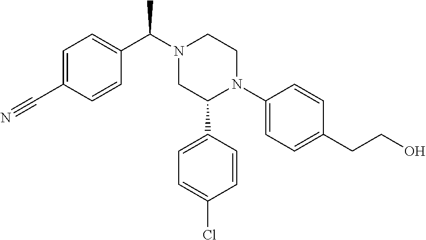 [SMILES: C[C@H](c1ccc(C#N)cc1)N1CCN(c2ccc(CCO)cc2)[C@H](c2ccc(Cl)cc2)C1] | 446.2 | 3.17 |
| 893 | 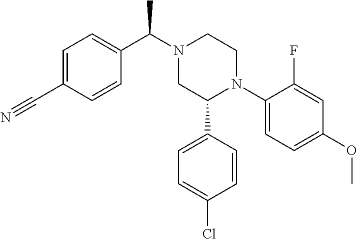 [SMILES: COc1ccc(N2CCN([C@H](C)c3ccc(C#N)cc3)C[C@H]2c2ccc(Cl)cc2)c(F)c1] | 450.2 | 3.36 |
| 895 | 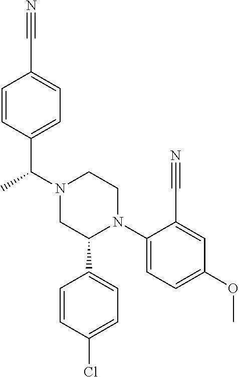 [SMILES: COc1ccc(N2CCN([C@H](C)c3ccc(C#N)cc3)C[C@H]2c2ccc(Cl)cc2)c(C#N)c1] | 457.3 | 3.61 |
| 896 | 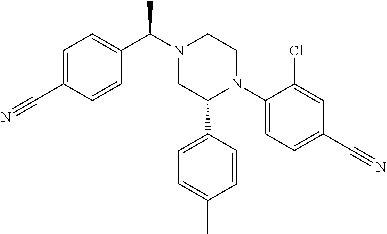 [SMILES: Cc1ccc([C@@H]2CN([C@H](C)c3ccc(C#N)cc3)CCN2c2ccc(C#N)cc2Cl)cc1] | 441.2 | 3.63 |
| 897 | 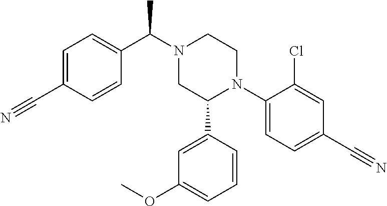 [SMILES: COc1cccc([C@@H]2CN([C@H](C)c3ccc(C#N)cc3)CCN2c2ccc(C#N)cc2Cl)c1] | 457.3 | 3.37 |
| 899 | 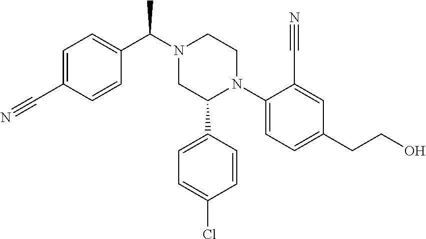 [SMILES: C[C@H](c1ccc(C#N)cc1)N1CCN(c2ccc(CCO)cc2C#N)[C@H](c2ccc(Cl)cc2)C1] | 471.3 | 3.04 |
| 899b | 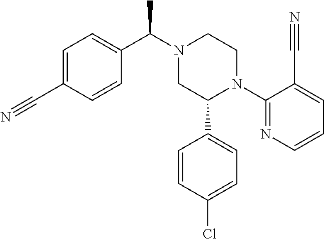 [SMILES: C[C@H](c1ccc(C#N)cc1)N1CCN(c2ncccc2C#N)[C@H](c2ccc(Cl)cc2)C1] | 428.2 | 3.38 |
| 900 | 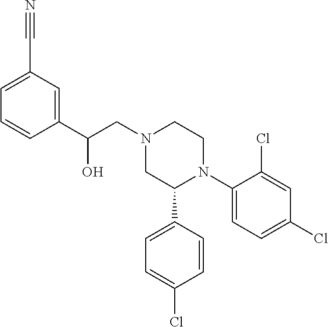 [SMILES: N#Cc1cccc(C(O)CN2CCN(c3ccc(Cl)cc3Cl)[C@H](c3ccc(Cl)cc3)C2)c1] | 486.3 | 3.51 |
| 901 | 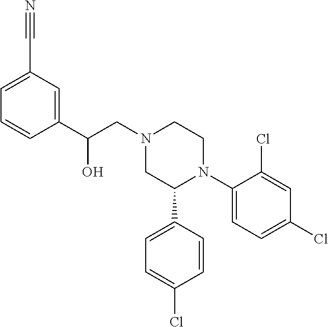 [SMILES: N#Cc1cccc(C(O)CN2CCN(c3ccc(Cl)cc3Cl)[C@H](c3ccc(Cl)cc3)C2)c1] | 486.3 | 3.51 |
| 902 | 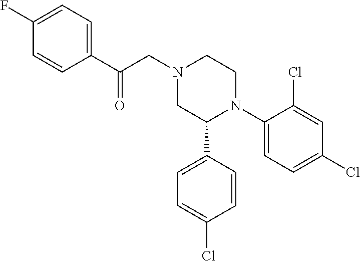 [SMILES: O=C(CN1CCN(c2ccc(Cl)cc2Cl)[C@H](c2ccc(Cl)cc2)C1)c1ccc(F)cc1] | 477.3 | 3.62 |
| 903 | 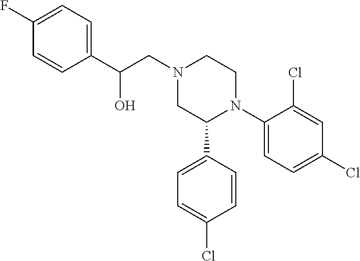 [SMILES: OC(CN1CCN(c2ccc(Cl)cc2Cl)[C@H](c2ccc(Cl)cc2)C1)c1ccc(F)cc1] | 479.3 | 3.58 |
| 904 | 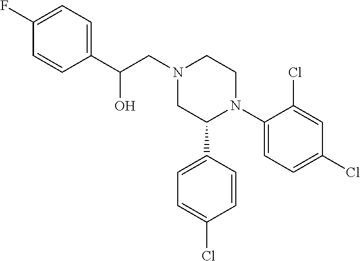 [SMILES: OC(CN1CCN(c2ccc(Cl)cc2Cl)[C@H](c2ccc(Cl)cc2)C1)c1ccc(F)cc1] | 479.3 | 3.56 |
| 905 | 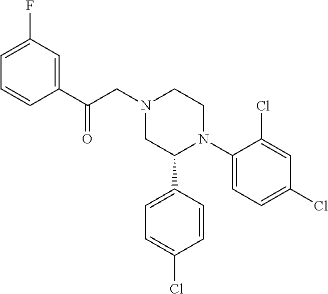 [SMILES: O=C(CN1CCN(c2ccc(Cl)cc2Cl)[C@H](c2ccc(Cl)cc2)C1)c1cccc(F)c1] | 477.3 | 3.80 |
| 906 | 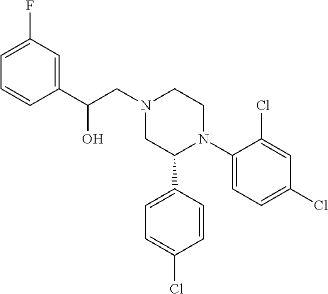 [SMILES: OC(CN1CCN(c2ccc(Cl)cc2Cl)[C@H](c2ccc(Cl)cc2)C1)c1cccc(F)c1] | 479.3 | 4.12 |
| 907 | 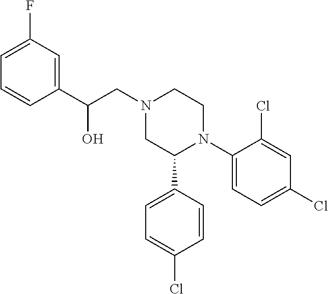 [SMILES: OC(CN1CCN(c2ccc(Cl)cc2Cl)[C@H](c2ccc(Cl)cc2)C1)c1cccc(F)c1] | 479.3 | 4.08 |
| 908 | 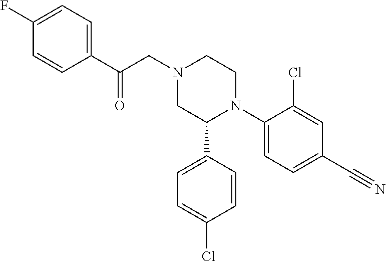 [SMILES: N#Cc1ccc(N2CCN(CC(=O)c3ccc(F)cc3)C[C@H]2c2ccc(Cl)cc2)c(Cl)c1] | 468.3 | 3.50 |
| 909 | 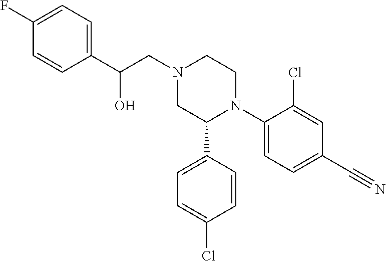 [SMILES: N#Cc1ccc(N2CCN(CC(O)c3ccc(F)cc3)C[C@H]2c2ccc(Cl)cc2)c(Cl)c1] | 470.3 | 3.21 |
| 910 | 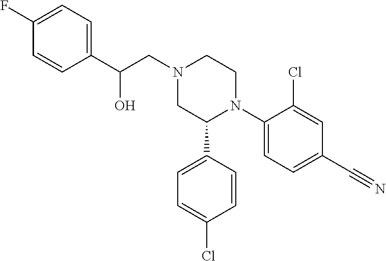 [SMILES: N#Cc1ccc(N2CCN(CC(O)c3ccc(F)cc3)C[C@H]2c2ccc(Cl)cc2)c(Cl)c1] | 470.3 | 3.26 |
| 911 | 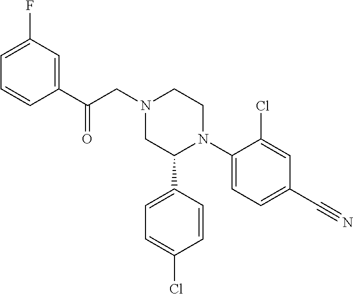 [SMILES: N#Cc1ccc(N2CCN(CC(=O)c3cccc(F)c3)C[C@H]2c2ccc(Cl)cc2)c(Cl)c1] | 468.3 | 3.55 |
| 912 | 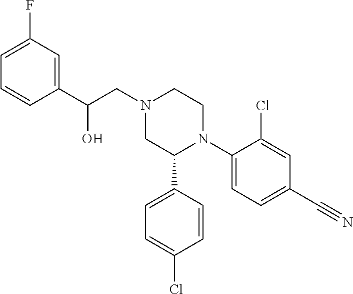 [SMILES: N#Cc1ccc(N2CCN(CC(O)c3cccc(F)c3)C[C@H]2c2ccc(Cl)cc2)c(Cl)c1] | 470.3 | 3.27 |
| 913 | 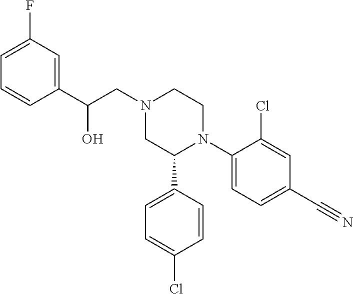 [SMILES: N#Cc1ccc(N2CCN(CC(O)c3cccc(F)c3)C[C@H]2c2ccc(Cl)cc2)c(Cl)c1] | 470.3 | 3.25 |
| 915 | 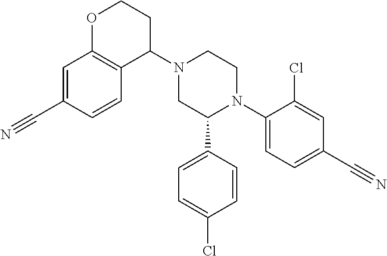 [SMILES: N#Cc1ccc(N2CCN(C3CCOc4cc(C#N)ccc43)C[C@H]2c2ccc(Cl)cc2)c(Cl)c1] | 489.3 | 4.68 |
| 916 | 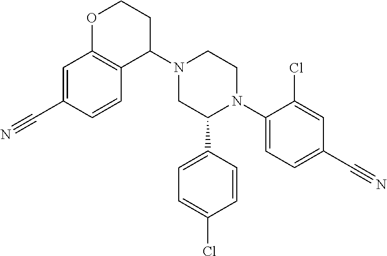 [SMILES: N#Cc1ccc(N2CCN(C3CCOc4cc(C#N)ccc43)C[C@H]2c2ccc(Cl)cc2)c(Cl)c1] | 489.3 | 4.68 |
| 918 | 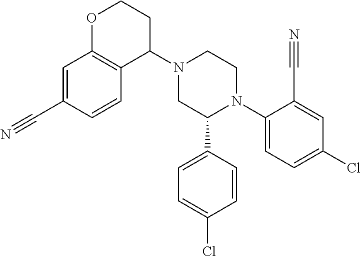 [SMILES: N#Cc1ccc2c(c1)OCCC2N1CCN(c2ccc(Cl)cc2C#N)[C@H](c2ccc(Cl)cc2)C1] | 489.3 | 4.67 |
| 919 | 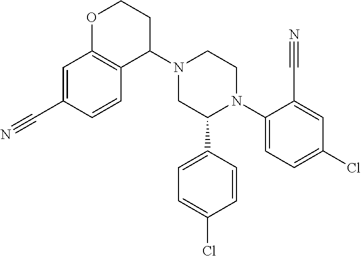 [SMILES: N#Cc1ccc2c(c1)OCCC2N1CCN(c2ccc(Cl)cc2C#N)[C@H](c2ccc(Cl)cc2)C1] | 489.3 | 4.67 |
| 920 | 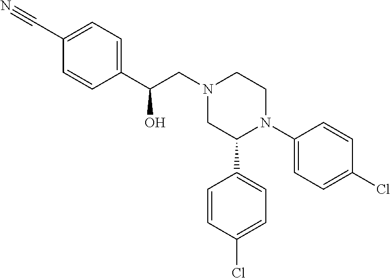 [SMILES: N#Cc1ccc([C@H](O)CN2CCN(c3ccc(Cl)cc3)[C@H](c3ccc(Cl)cc3)C2)cc1] | 452.2 | 3.24 |
| 921 | 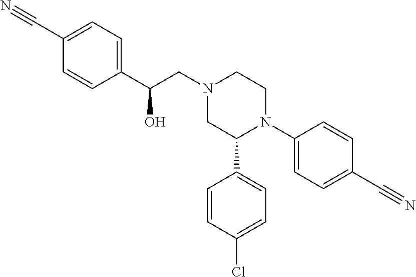 [SMILES: N#Cc1ccc([C@H](O)CN2CCN(c3ccc(C#N)cc3)[C@H](c3ccc(Cl)cc3)C2)cc1] | 443.2 | 3.39 |
| 922 | 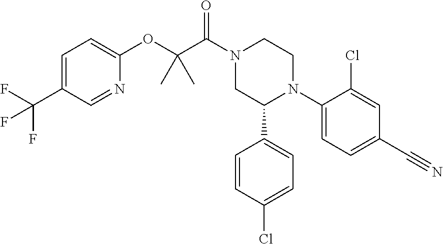 [SMILES: CC(C)(Oc1ccc(C(F)(F)F)cn1)C(=O)N1CCN(c2ccc(C#N)cc2Cl)[C@H](c2ccc(Cl)cc2)C1] | 563.3 | 5.36 |
| 923 | 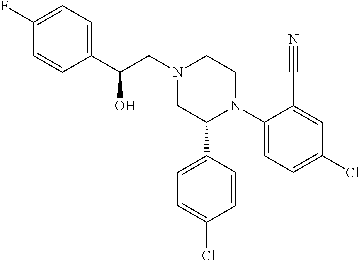 [SMILES: N#Cc1cc(Cl)ccc1N1CCN(C[C@@H](O)c2ccc(F)cc2)C[C@H]1c1ccc(Cl)cc1] | 470.3 | 3.45 |
| 923a | 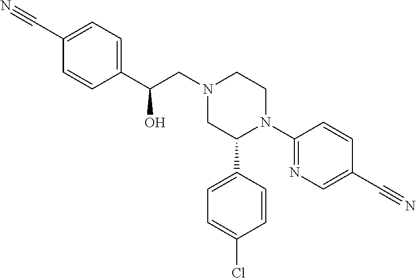 [SMILES: N#Cc1ccc([C@H](O)CN2CCN(c3ccc(C#N)cn3)[C@H](c3ccc(Cl)cc3)C2)cc1] | 444.2 | 3.03 |
| 923b | 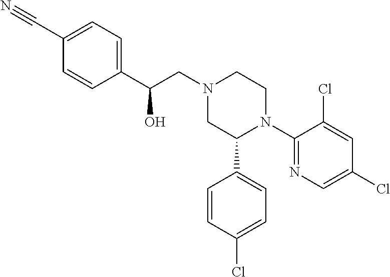 [SMILES: N#Cc1ccc([C@H](O)CN2CCN(c3ncc(Cl)cc3Cl)[C@H](c3ccc(Cl)cc3)C2)cc1] | 489.3 | 3.26 |
| 923c | 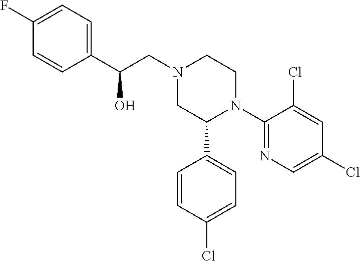 [SMILES: O[C@H](CN1CCN(c2ncc(Cl)cc2Cl)[C@H](c2ccc(Cl)cc2)C1)c1ccc(F)cc1] | 480.3 | 3.92 |
| 924 | 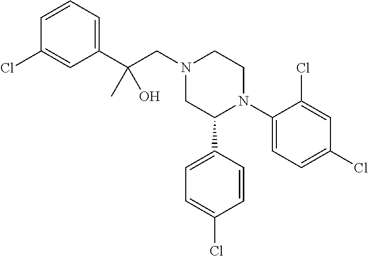 [SMILES: CC(O)(CN1CCN(c2ccc(Cl)cc2Cl)[C@H](c2ccc(Cl)cc2)C1)c1cccc(Cl)c1] | 511.3 | 3.85 |
| 925 | 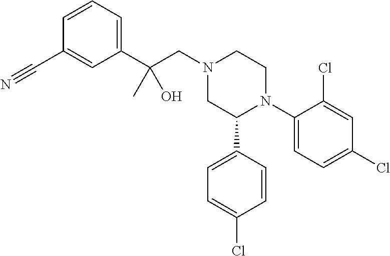 [SMILES: CC(O)(CN1CCN(c2ccc(Cl)cc2Cl)[C@H](c2ccc(Cl)cc2)C1)c1cccc(C#N)c1] | 502.3 | 3.64 |
| 926 | 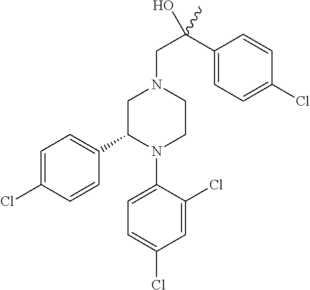 [SMILES: CC(O)(CN1CCN(c2ccc(Cl)cc2Cl)[C@H](c2ccc(Cl)cc2)C1)c1ccc(Cl)cc1] | 511.3 | 3.86 |
| 927 | 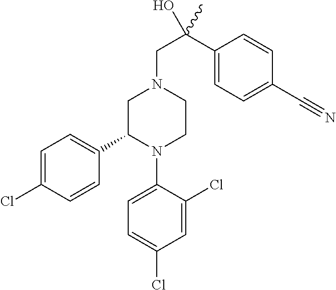 [SMILES: CC(O)(CN1CCN(c2ccc(Cl)cc2Cl)[C@H](c2ccc(Cl)cc2)C1)c1ccc(C#N)cc1] | 500.3 | 3.77 |
| 928 | 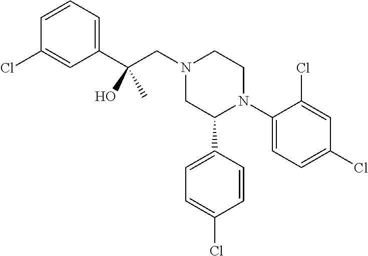 [SMILES: C[C@@](O)(CN1CCN(c2ccc(Cl)cc2Cl)[C@H](c2ccc(Cl)cc2)C1)c1cccc(Cl)c1] | 511.3 | 4.49 |
| 929 | 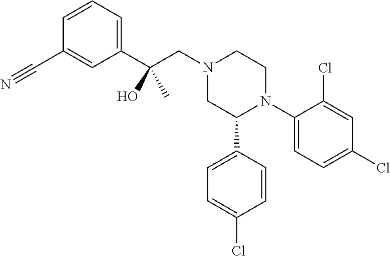 [SMILES: C[C@@](O)(CN1CCN(c2ccc(Cl)cc2Cl)[C@H](c2ccc(Cl)cc2)C1)c1cccc(C#N)c1] | 502.3 | 3.78 |
| 930 | 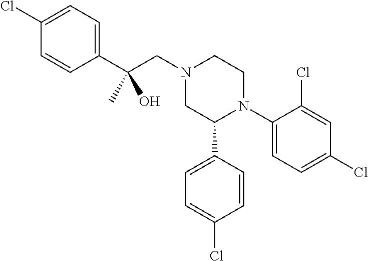 [SMILES: C[C@@](O)(CN1CCN(c2ccc(Cl)cc2Cl)[C@H](c2ccc(Cl)cc2)C1)c1ccc(Cl)cc1] | 511.3 | 3.72 |
| 931 | 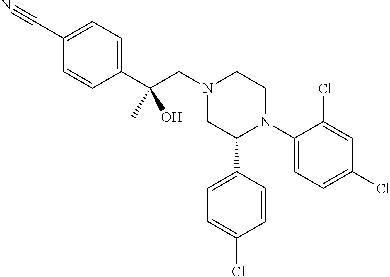 [SMILES: C[C@@](O)(CN1CCN(c2ccc(Cl)cc2Cl)[C@H](c2ccc(Cl)cc2)C1)c1ccc(C#N)cc1] | 502. | 3.81 |
| 932 | 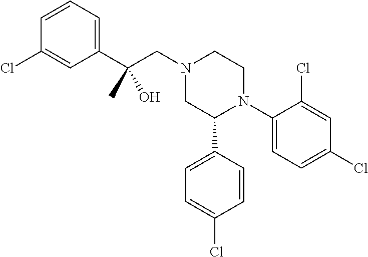 [SMILES: C[C@](O)(CN1CCN(c2ccc(Cl)cc2Cl)[C@H](c2ccc(Cl)cc2)C1)c1cccc(Cl)c1] | 511.3 | 3.84 |
| 933 | 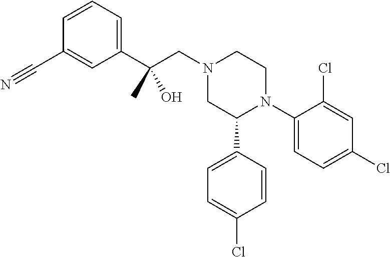 [SMILES: C[C@](O)(CN1CCN(c2ccc(Cl)cc2Cl)[C@H](c2ccc(Cl)cc2)C1)c1cccc(C#N)c1] | 502.3 | 4.17 |
| 934 | 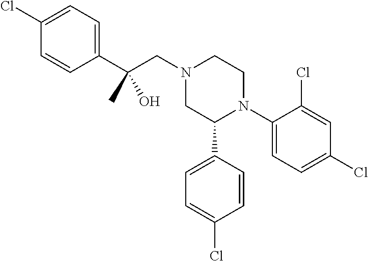 [SMILES: C[C@](O)(CN1CCN(c2ccc(Cl)cc2Cl)[C@H](c2ccc(Cl)cc2)C1)c1ccc(Cl)cc1] | 511.3 | 3.99 |
| 935 | 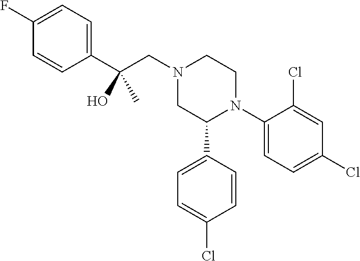 [SMILES: C[C@@](O)(CN1CCN(c2ccc(Cl)cc2Cl)[C@H](c2ccc(Cl)cc2)C1)c1ccc(F)cc1] | 493.3 | 3.87 |
| 936 | 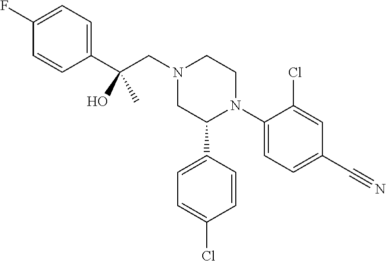 [SMILES: C[C@@](O)(CN1CCN(c2ccc(C#N)cc2Cl)[C@H](c2ccc(Cl)cc2)C1)c1ccc(F)cc1] | 484.3 | 3.54 |
| 937 | 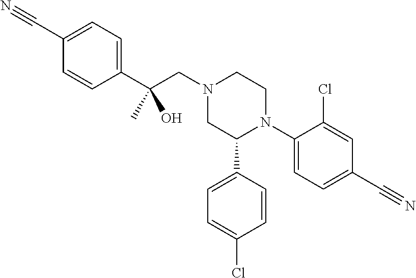 [SMILES: C[C@@](O)(CN1CCN(c2ccc(C#N)cc2Cl)[C@H](c2ccc(Cl)cc2)C1)c1ccc(C#N)cc1] | 491.3 | 3.18 |
| 938 | 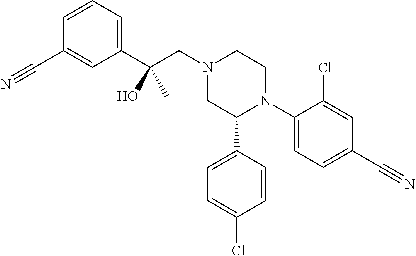 [SMILES: C[C@@](O)(CN1CCN(c2ccc(C#N)cc2Cl)[C@H](c2ccc(Cl)cc2)C1)c1cccc(C#N)c1] | 491.3 | 3.24 |
| 939 | 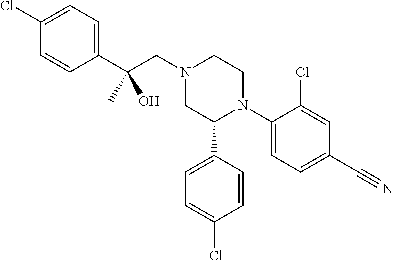 [SMILES: C[C@@](O)(CN1CCN(c2ccc(C#N)cc2Cl)[C@H](c2ccc(Cl)cc2)C1)c1ccc(Cl)cc1] | 502.3 | 3.41 |
| 940 | 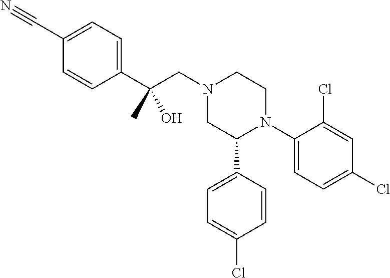 [SMILES: C[C@](O)(CN1CCN(c2ccc(Cl)cc2Cl)[C@H](c2ccc(Cl)cc2)C1)c1ccc(C#N)cc1] | 502.3 | 3.46 |
| 941 | 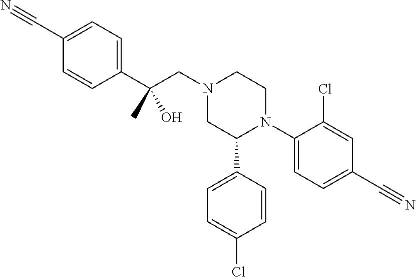 [SMILES: C[C@](O)(CN1CCN(c2ccc(C#N)cc2Cl)[C@H](c2ccc(Cl)cc2)C1)c1ccc(C#N)cc1] | 491.3 | 3.16 |
| 942 | 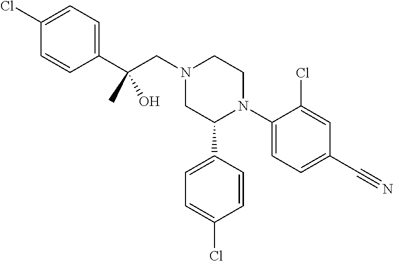 [SMILES: C[C@](O)(CN1CCN(c2ccc(C#N)cc2Cl)[C@H](c2ccc(Cl)cc2)C1)c1ccc(Cl)cc1] | 500.3 | 3.62 |
| 943 | 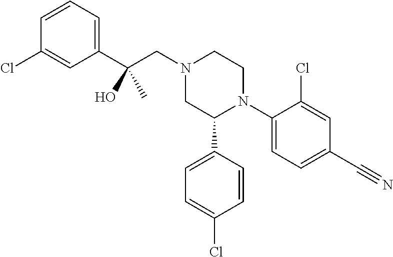 [SMILES: C[C@@](O)(CN1CCN(c2ccc(C#N)cc2Cl)[C@H](c2ccc(Cl)cc2)C1)c1cccc(Cl)c1] | 502.3 | 3.62 |
| 944 | 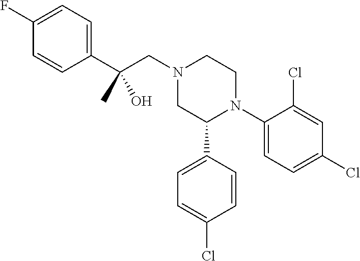 [SMILES: C[C@](O)(CN1CCN(c2ccc(Cl)cc2Cl)[C@H](c2ccc(Cl)cc2)C1)c1ccc(F)cc1] | 495.3 | 3.68 |
| 945 | 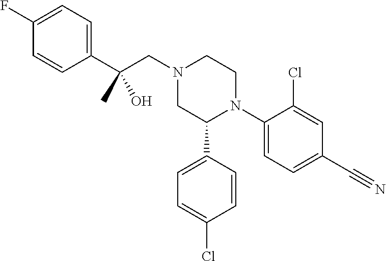 [SMILES: C[C@](O)(CN1CCN(c2ccc(C#N)cc2Cl)[C@H](c2ccc(Cl)cc2)C1)c1ccc(F)cc1] | 484.3 | 3.39 |
| 946 | 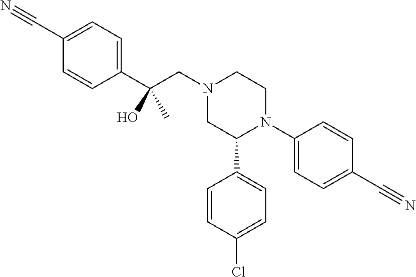 [SMILES: C[C@@](O)(CN1CCN(c2ccc(C#N)cc2)[C@H](c2ccc(Cl)cc2)C1)c1ccc(C#N)cc1] | 457.3 | 3.29 |
| 947 | 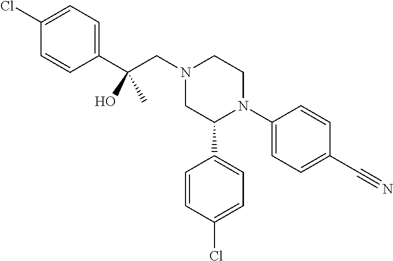 [SMILES: C[C@@](O)(CN1CCN(c2ccc(C#N)cc2)[C@H](c2ccc(Cl)cc2)C1)c1ccc(Cl)cc1] | 466.3 | 3.49 |
| 948 | 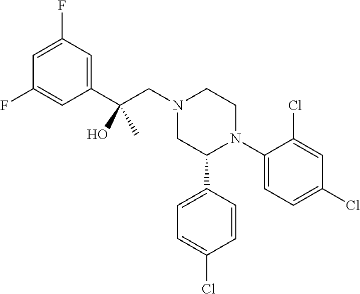 [SMILES: C[C@@](O)(CN1CCN(c2ccc(Cl)cc2Cl)[C@H](c2ccc(Cl)cc2)C1)c1cc(F)cc(F)c1] | 511.3 | 4.36 |
| 949 | 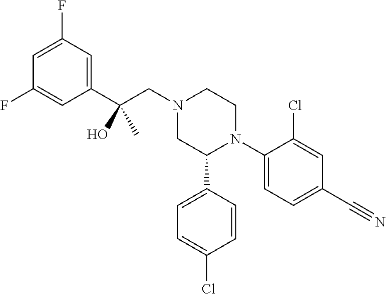 [SMILES: C[C@@](O)(CN1CCN(c2ccc(C#N)cc2Cl)[C@H](c2ccc(Cl)cc2)C1)c1cc(F)cc(F)c1] | 502.3 | 3.96 |
| 950 | 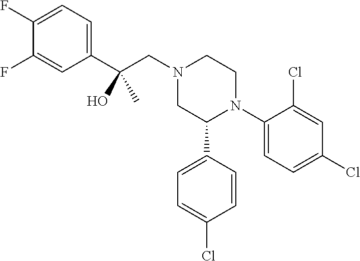 [SMILES: C[C@@](O)(CN1CCN(c2ccc(Cl)cc2Cl)[C@H](c2ccc(Cl)cc2)C1)c1ccc(F)c(F)c1] | 513.3 | 4.41 |
| 951 | 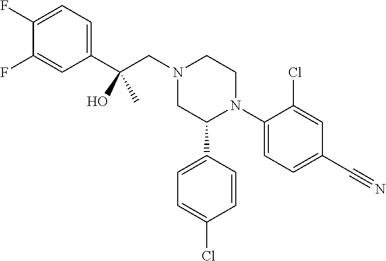 [SMILES: C[C@@](O)(CN1CCN(c2ccc(C#N)cc2Cl)[C@H](c2ccc(Cl)cc2)C1)c1ccc(F)c(F)c1] | 502.3 | 3.92 |
| 952 | 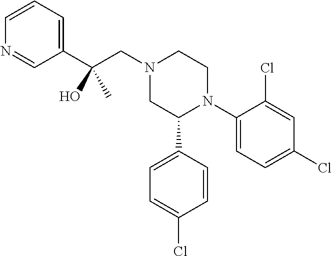 [SMILES: C[C@@](O)(CN1CCN(c2ccc(Cl)cc2Cl)[C@H](c2ccc(Cl)cc2)C1)c1cccnc1] | 478.3 | 2.64 |
| 953 | 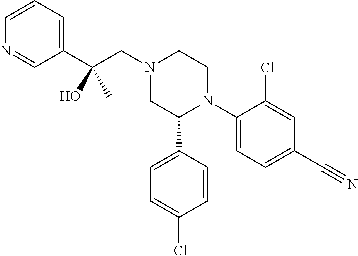 [SMILES: C[C@@](O)(CN1CCN(c2ccc(C#N)cc2Cl)[C@H](c2ccc(Cl)cc2)C1)c1cccnc1] | 467.3 | 2.83 |
| 954 | 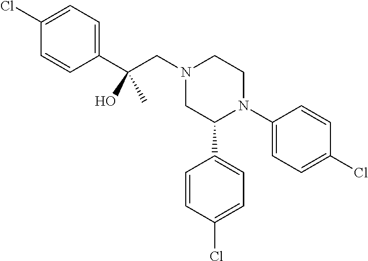 [SMILES: C[C@@](O)(CN1CCN(c2ccc(Cl)cc2)[C@H](c2ccc(Cl)cc2)C1)c1ccc(Cl)cc1] | 475.3 | 3.53 |
| 955 | 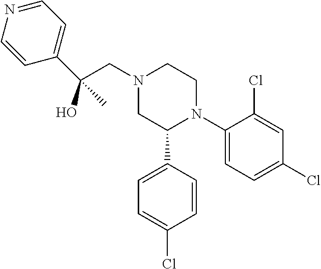 [SMILES: C[C@@](O)(CN1CCN(c2ccc(Cl)cc2Cl)[C@H](c2ccc(Cl)cc2)C1)c1ccncc1] | 476.3 | 2.88 |
| 956 | 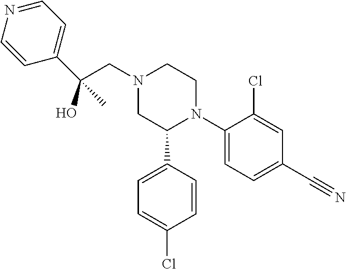 [SMILES: C[C@@](O)(CN1CCN(c2ccc(C#N)cc2Cl)[C@H](c2ccc(Cl)cc2)C1)c1ccncc1] | 467.3 | 2.63 |
| 957 | 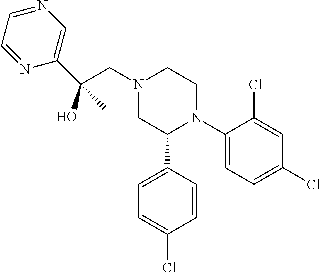 [SMILES: C[C@@](O)(CN1CCN(c2ccc(Cl)cc2Cl)[C@H](c2ccc(Cl)cc2)C1)c1cnccn1] | 479.3 | 3.18 |
| 958 | 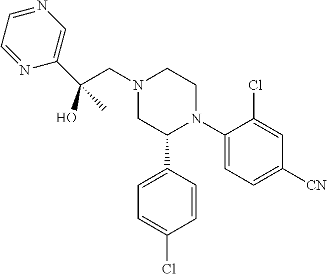 [SMILES: C[C@@](O)(CN1CCN(c2ccc(C#N)cc2Cl)[C@H](c2ccc(Cl)cc2)C1)c1cnccn1] | 468.3 | 2.89 |
| 959 | 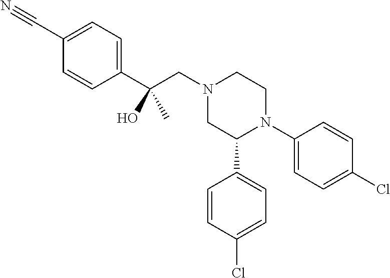 [SMILES: C[C@@](O)(CN1CCN(c2ccc(Cl)cc2)[C@H](c2ccc(Cl)cc2)C1)c1ccc(C#N)cc1] | 466.3 | 3.29 |
| 960 | 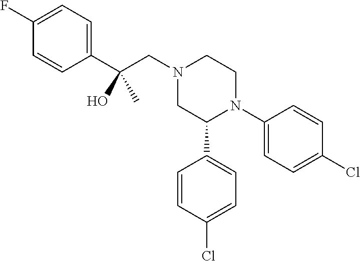 [SMILES: C[C@@](O)(CN1CCN(c2ccc(Cl)cc2)[C@H](c2ccc(Cl)cc2)C1)c1ccc(F)cc1] | 459.3 | 3.83 |
| 961 | 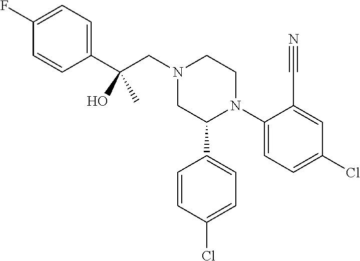 [SMILES: C[C@@](O)(CN1CCN(c2ccc(Cl)cc2C#N)[C@H](c2ccc(Cl)cc2)C1)c1ccc(F)cc1] | 484.3 | 3.75 |
| 962 | 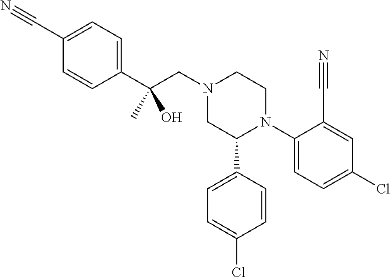 [SMILES: C[C@@](O)(CN1CCN(c2ccc(Cl)cc2C#N)[C@H](c2ccc(Cl)cc2)C1)c1ccc(C#N)cc1] | 491.3 | 3.54 |
| 963 | 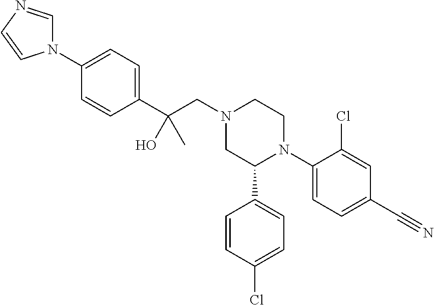 [SMILES: CC(O)(CN1CCN(c2ccc(C#N)cc2Cl)[C@H](c2ccc(Cl)cc2)C1)c1ccc(-n2ccnc2)cc1] | 532.3 | 2.48 |
| 964 | 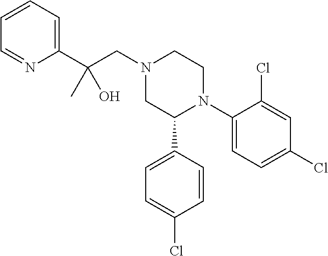 [SMILES: CC(O)(CN1CCN(c2ccc(Cl)cc2Cl)[C@H](c2ccc(Cl)cc2)C1)c1ccccn1] | 478.3 | 3.81 |
| 965a | 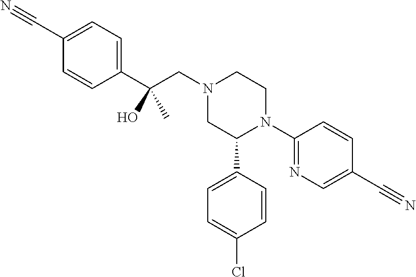 [SMILES: C[C@@](O)(CN1CCN(c2ccc(C#N)cn2)[C@H](c2ccc(Cl)cc2)C1)c1ccc(C#N)cc1] | 458.3 | 3.47 |
| 965b | 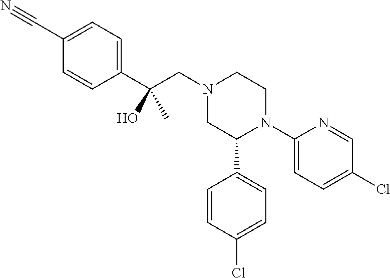 [SMILES: C[C@@](O)(CN1CCN(c2ccc(Cl)cn2)[C@H](c2ccc(Cl)cc2)C1)c1ccc(C#N)cc1] | 467.3 | 3.28 |
| 965c | 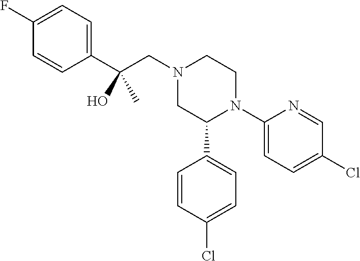 [SMILES: C[C@@](O)(CN1CCN(c2ccc(Cl)cn2)[C@H](c2ccc(Cl)cc2)C1)c1ccc(F)cc1] | 460.3 | 3.33 |
| 965d | 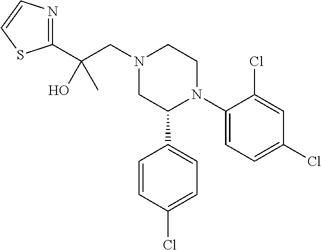 [SMILES: CC(O)(CN1CCN(c2ccc(Cl)cc2Cl)[C@H](c2ccc(Cl)cc2)C1)c1nccs1] | 482.3 | 3.26 |
| 965e | 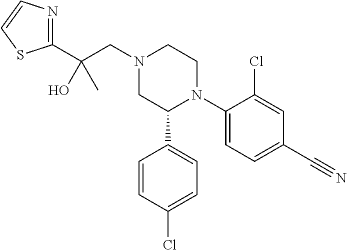 [SMILES: CC(O)(CN1CCN(c2ccc(C#N)cc2Cl)[C@H](c2ccc(Cl)cc2)C1)c1nccs1] | 473.3 | 2.98 |
| 965f | 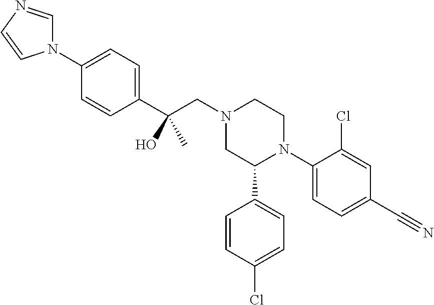 [SMILES: C[C@@](O)(CN1CCN(c2ccc(C#N)cc2Cl)[C@H](c2ccc(Cl)cc2)C1)c1ccc(-n2ccnc2)cc1] | 532.3 | 2.79 |
| 966 | 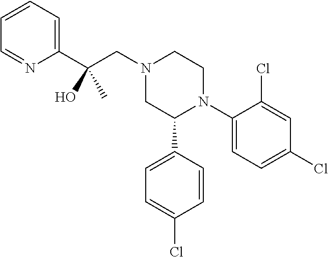 [SMILES: C[C@@](O)(CN1CCN(c2ccc(Cl)cc2Cl)[C@H](c2ccc(Cl)cc2)C1)c1ccccn1] | 476.3 | 3.88 |
| 967 | 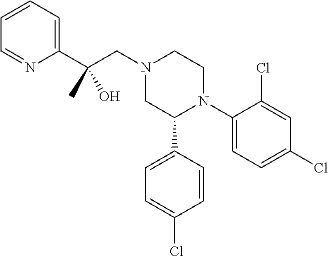 [SMILES: C[C@](O)(CN1CCN(c2ccc(Cl)cc2Cl)[C@H](c2ccc(Cl)cc2)C1)c1ccccn1] | 476.3 | 3.84 |
| 968 | 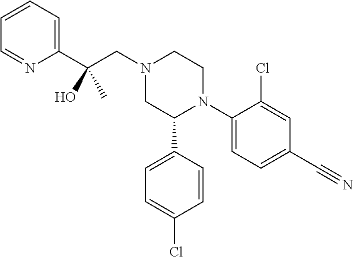 [SMILES: C[C@@](O)(CN1CCN(c2ccc(C#N)cc2Cl)[C@H](c2ccc(Cl)cc2)C1)c1ccccn1] | 467.3 | 2.99 |
| 969 | 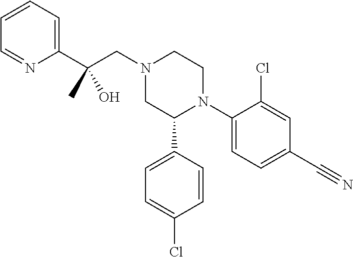 [SMILES: C[C@](O)(CN1CCN(c2ccc(C#N)cc2Cl)[C@H](c2ccc(Cl)cc2)C1)c1ccccn1] | 467.3 | 2.96 |
| 970 | 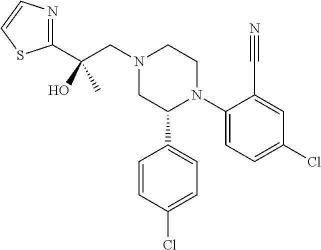 [SMILES: C[C@@](O)(CN1CCN(c2ccc(Cl)cc2C#N)[C@H](c2ccc(Cl)cc2)C1)c1nccs1] | 473.3 | 3.03 |
| 971 | 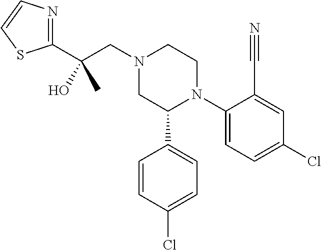 [SMILES: C[C@](O)(CN1CCN(c2ccc(Cl)cc2C#N)[C@H](c2ccc(Cl)cc2)C1)c1nccs1] | 473.3 | 3.04 |
| 972 | 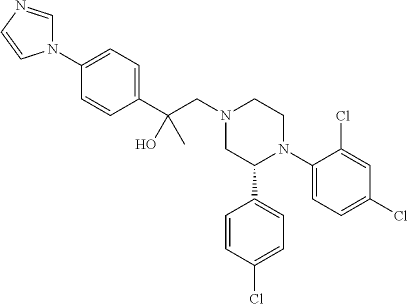 [SMILES: CC(O)(CN1CCN(c2ccc(Cl)cc2Cl)[C@H](c2ccc(Cl)cc2)C1)c1ccc(-n2ccnc2)cc1] | 541.3 | 2.59 |
| 973 | 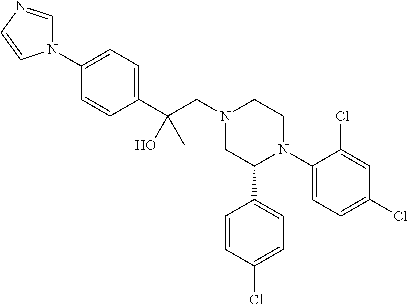 [SMILES: CC(O)(CN1CCN(c2ccc(Cl)cc2Cl)[C@H](c2ccc(Cl)cc2)C1)c1ccc(-n2ccnc2)cc1] | 541.3 | 2.58 |
| 974 | 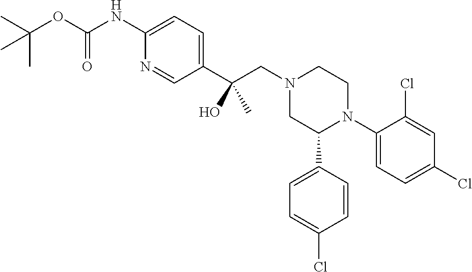 [SMILES: CC(C)(C)OC(=O)Nc1ccc([C@](C)(O)CN2CCN(c3ccc(Cl)cc3Cl)[C@H](c3ccc(Cl)cc3)C2)cn1] | 537.3 | 3.64 |
| 975 | 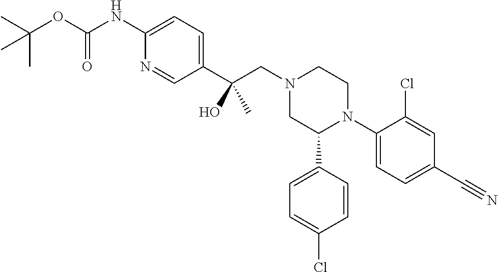 [SMILES: CC(C)(C)OC(=O)Nc1ccc([C@](C)(O)CN2CCN(c3ccc(C#N)cc3Cl)[C@H](c3ccc(Cl)cc3)C2)cn1] | 526.3 | 3.33 |
| 976 | 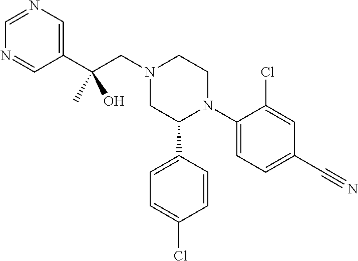 [SMILES: C[C@@](O)(CN1CCN(c2ccc(C#N)cc2Cl)[C@H](c2ccc(Cl)cc2)C1)c1cncnc1] | 468.3 | 2.70 |
| 977 | 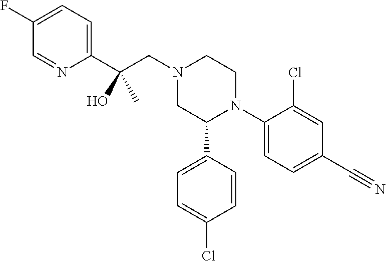 [SMILES: C[C@@](O)(CN1CCN(c2ccc(C#N)cc2Cl)[C@H](c2ccc(Cl)cc2)C1)c1ccc(F)cn1] | 485.3 | 3.15 |
| 978 | 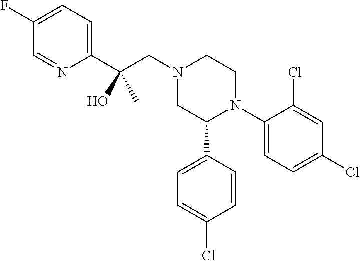 [SMILES: C[C@@](O)(CN1CCN(c2ccc(Cl)cc2Cl)[C@H](c2ccc(Cl)cc2)C1)c1ccc(F)cn1] | 496.3 | 3.47 |
| 979 | 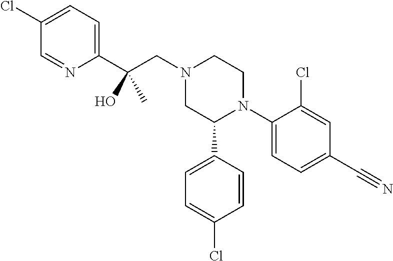 [SMILES: C[C@@](O)(CN1CCN(c2ccc(C#N)cc2Cl)[C@H](c2ccc(Cl)cc2)C1)c1ccc(Cl)cn1] | 503.3 | 3.91 |
| 980 | 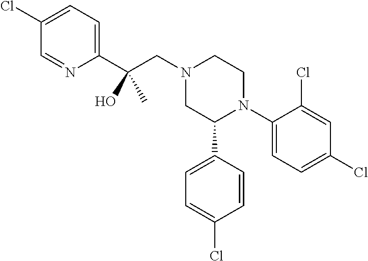 [SMILES: C[C@@](O)(CN1CCN(c2ccc(Cl)cc2Cl)[C@H](c2ccc(Cl)cc2)C1)c1ccc(Cl)cn1] | 512.3 | 4.41 |
| 981 | 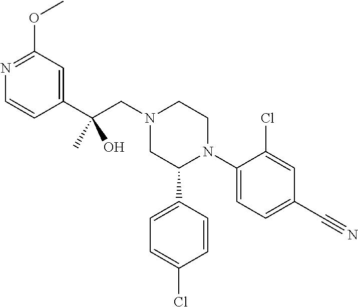 [SMILES: COc1cc([C@](C)(O)CN2CCN(c3ccc(C#N)cc3Cl)[C@H](c3ccc(Cl)cc3)C2)ccn1] | 497.2 | 3.35 |
| 982 | 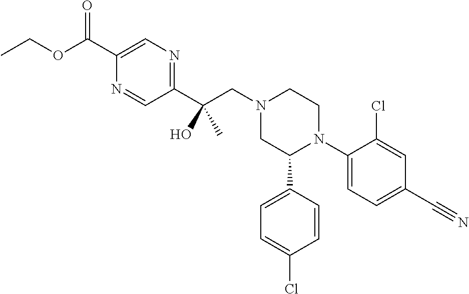 [SMILES: CCOC(=O)c1cnc([C@](C)(O)CN2CCN(c3ccc(C#N)cc3Cl)[C@H](c3ccc(Cl)cc3)C2)cn1] | 540.3 | 3.14 |
| 983 | 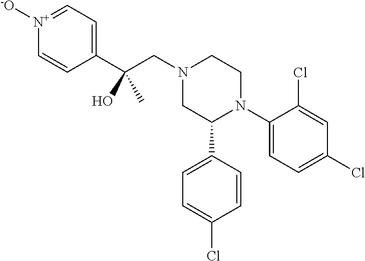 | 492.3 | 2.85 |
| 984 | 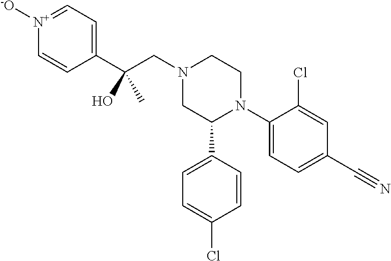 | 483.3 | 2.55 |
| 985 | 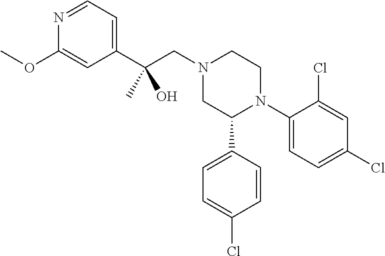 [SMILES: COc1cc([C@](C)(O)CN2CCN(c3ccc(Cl)cc3Cl)[C@H](c3ccc(Cl)cc3)C2)ccn1] | 508.3 | 3.24 |
| 986 | 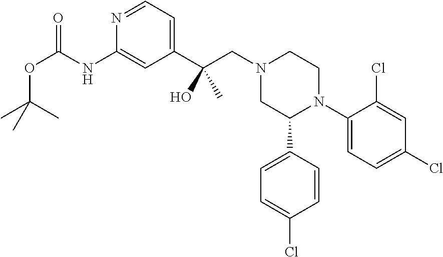 [SMILES: CC(C)(C)OC(=O)Nc1cc([C@](C)(O)CN2CCN(c3ccc(Cl)cc3Cl)[C@H](c3ccc(Cl)cc3)C2)ccn1] | 593.3 | 3.89 |
| 987 | 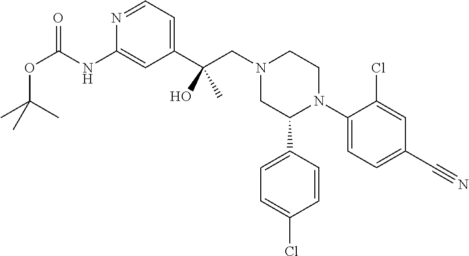 [SMILES: CC(C)(C)OC(=O)Nc1cc([C@](C)(O)CN2CCN(c3ccc(C#N)cc3Cl)[C@H](c3ccc(Cl)cc3)C2)ccn1] | 582.3 | 3.48 |
| 988 | 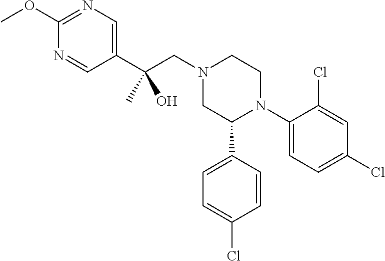 [SMILES: COc1ncc([C@](C)(O)CN2CCN(c3ccc(Cl)cc3Cl)[C@H](c3ccc(Cl)cc3)C2)cn1] | 509.3 | 3.61 |
| 989 | 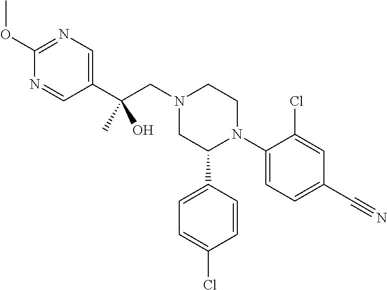 [SMILES: COc1ncc([C@](C)(O)CN2CCN(c3ccc(C#N)cc3Cl)[C@H](c3ccc(Cl)cc3)C2)cn1] | 498.3 | 3.22 |
| 990 | 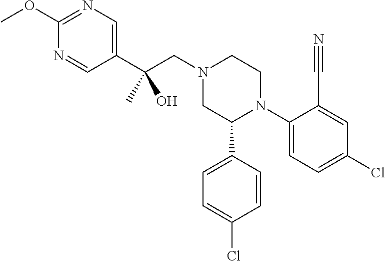 [SMILES: COc1ncc([C@](C)(O)CN2CCN(c3ccc(Cl)cc3C#N)[C@H](c3ccc(Cl)cc3)C2)cn1] | 498.3 | 3.19 |
| 991 | 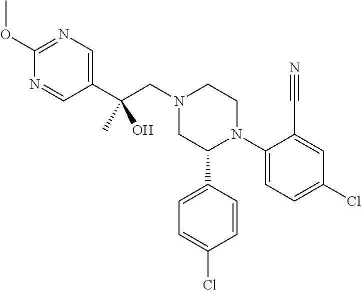 [SMILES: COc1ncc([C@](C)(O)CN2CCN(c3ccc(Cl)cc3C#N)[C@H](c3ccc(Cl)cc3)C2)cn1] | 497.3 | 3.31 |
| 992 | 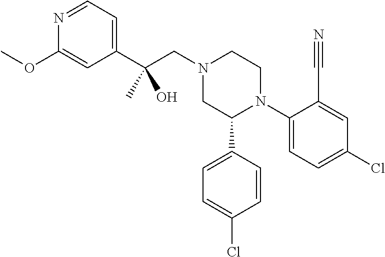 [SMILES: COc1cc([C@](C)(O)CN2CCN(c3ccc(Cl)cc3C#N)[C@H](c3ccc(Cl)cc3)C2)ccn1] | 497.3 | 3.37 |
| 993 | 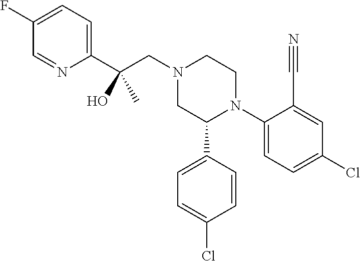 [SMILES: C[C@@](O)(CN1CCN(c2ccc(Cl)cc2C#N)[C@H](c2ccc(Cl)cc2)C1)c1ccc(F)cn1] | 485.3 | 3.51 |
| 994 | 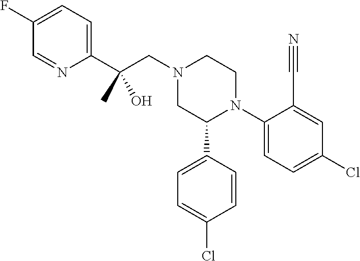 [SMILES: C[C@](O)(CN1CCN(c2ccc(Cl)cc2C#N)[C@H](c2ccc(Cl)cc2)C1)c1ccc(F)cn1] | 485.3 | 3.49 |
| 995 | 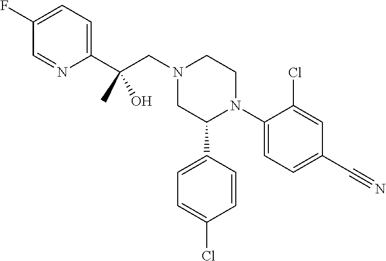 [SMILES: C[C@](O)(CN1CCN(c2ccc(C#N)cc2Cl)[C@H](c2ccc(Cl)cc2)C1)c1ccc(F)cn1] | 485.3 | 3.42 |
| 996 | 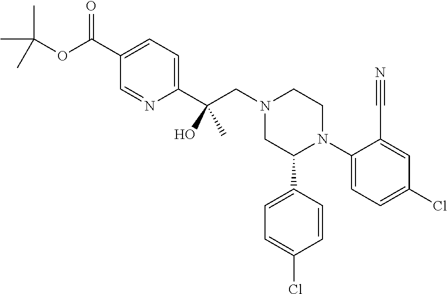 [SMILES: CC(C)(C)OC(=O)c1ccc([C@](C)(O)CN2CCN(c3ccc(Cl)cc3C#N)[C@H](c3ccc(Cl)cc3)C2)nc1] | 567.3 | 3.58 |
| 997 | 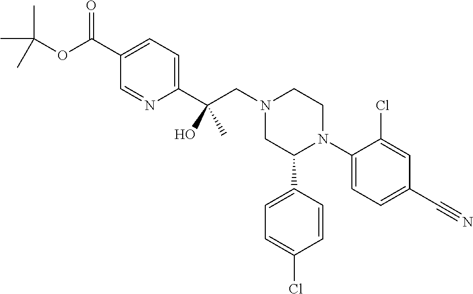 [SMILES: CC(C)(C)OC(=O)c1ccc([C@](C)(O)CN2CCN(c3ccc(C#N)cc3Cl)[C@H](c3ccc(Cl)cc3)C2)nc1] | 567.3 | 3.53 |
| 998 | 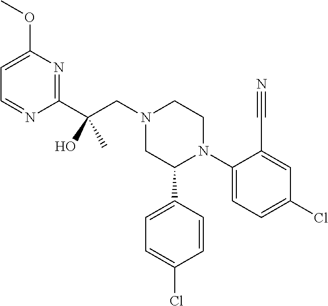 [SMILES: COc1ccnc([C@](C)(O)CN2CCN(c3ccc(Cl)cc3C#N)[C@H](c3ccc(Cl)cc3)C2)n1] | 498.3 | 3.69 |
| 999 | 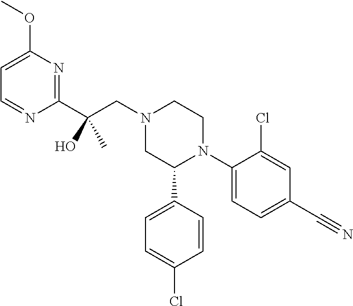 [SMILES: COc1ccnc([C@](C)(O)CN2CCN(c3ccc(C#N)cc3Cl)[C@H](c3ccc(Cl)cc3)C2)n1] | 498.3 | 3.59 |
| 1000 | 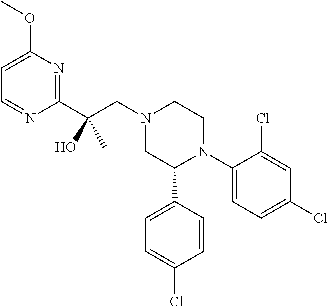 [SMILES: COc1ccnc([C@](C)(O)CN2CCN(c3ccc(Cl)cc3Cl)[C@H](c3ccc(Cl)cc3)C2)n1] | 509.3 | 4.15 |
| 1001 | 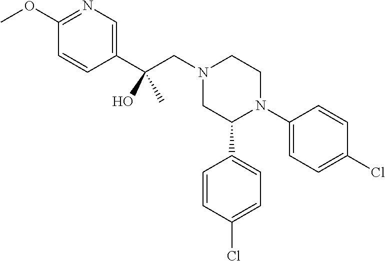 [SMILES: COc1ccc([C@](C)(O)CN2CCN(c3ccc(Cl)cc3)[C@H](c3ccc(Cl)cc3)C2)cn1] | 472.3 | 3.22 |
| 1002 | 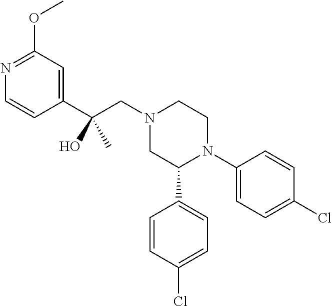 [SMILES: COc1cc([C@](C)(O)CN2CCN(c3ccc(Cl)cc3)[C@H](c3ccc(Cl)cc3)C2)ccn1] | 472.3 | 3.21 |
| 1003 | 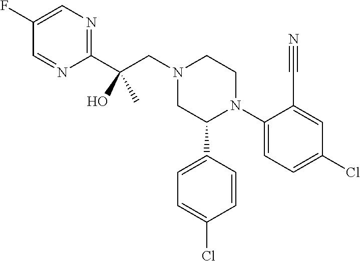 [SMILES: C[C@@](O)(CN1CCN(c2ccc(Cl)cc2C#N)[C@H](c2ccc(Cl)cc2)C1)c1ncc(F)cn1] | 486.3 | 3.06 |
| 1004 | 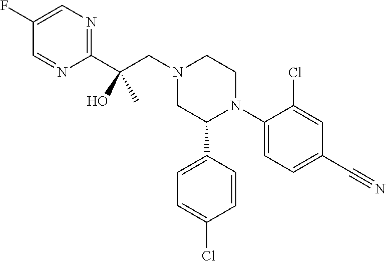 [SMILES: C[C@@](O)(CN1CCN(c2ccc(C#N)cc2Cl)[C@H](c2ccc(Cl)cc2)C1)c1ncc(F)cn1] | 486.3 | 2.99 |
| 1005 | 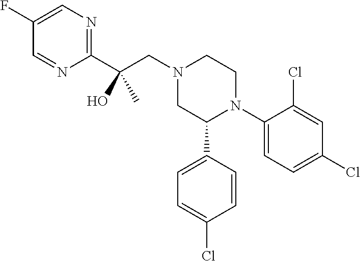 [SMILES: C[C@@](O)(CN1CCN(c2ccc(Cl)cc2Cl)[C@H](c2ccc(Cl)cc2)C1)c1ncc(F)cn1] | 495.3 | 3.29 |
| 1006 | 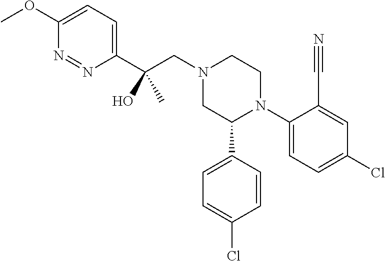 [SMILES: COc1ccc([C@](C)(O)CN2CCN(c3ccc(Cl)cc3C#N)[C@H](c3ccc(Cl)cc3)C2)nn1] | 498.3 | 3.23 |
| 1007 | 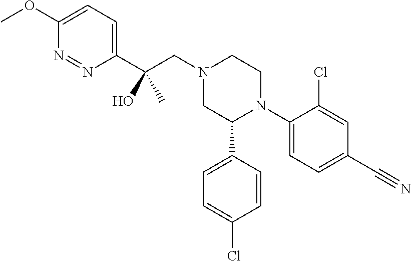 [SMILES: COc1ccc([C@](C)(O)CN2CCN(c3ccc(C#N)cc3Cl)[C@H](c3ccc(Cl)cc3)C2)nn1] | 498.3 | 3.15 |
| 1008 | 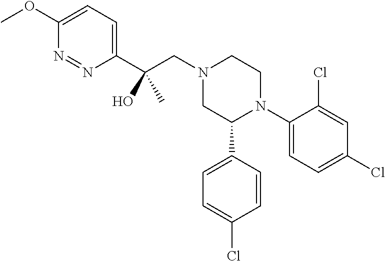 [SMILES: COc1ccc([C@](C)(O)CN2CCN(c3ccc(Cl)cc3Cl)[C@H](c3ccc(Cl)cc3)C2)nn1] | 509.3 | 3.45 |
| 1009 | 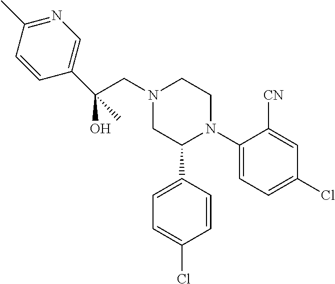 [SMILES: Cc1ccc([C@](C)(O)CN2CCN(c3ccc(Cl)cc3C#N)[C@H](c3ccc(Cl)cc3)C2)cn1] | 481.3 | 2.72 |
| 1010 | 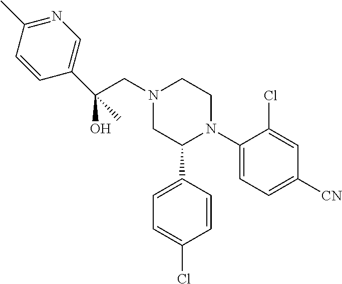 [SMILES: Cc1ccc([C@](C)(O)CN2CCN(c3ccc(C#N)cc3Cl)[C@H](c3ccc(Cl)cc3)C2)cn1] | 481.3 | 2.65 |
| 1011 | 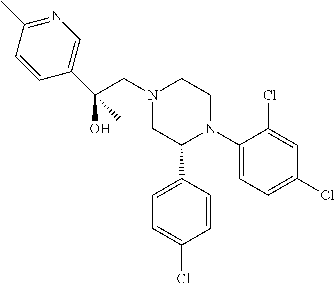 [SMILES: Cc1ccc([C@](C)(O)CN2CCN(c3ccc(Cl)cc3Cl)[C@H](c3ccc(Cl)cc3)C2)cn1] | 492.3 | 2.55 |
| 1012 | 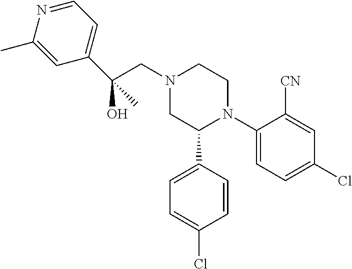 [SMILES: Cc1cc([C@](C)(O)CN2CCN(c3ccc(Cl)cc3C#N)[C@H](c3ccc(Cl)cc3)C2)ccn1] | 481.3 | 2.40 |
| 1013 | 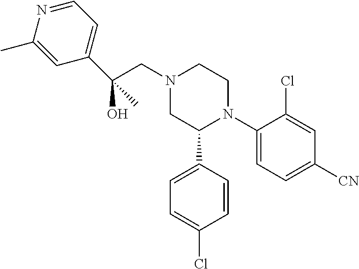 [SMILES: Cc1cc([C@](C)(O)CN2CCN(c3ccc(C#N)cc3Cl)[C@H](c3ccc(Cl)cc3)C2)ccn1] | 481.3 | 2.69 |
| 1014 | 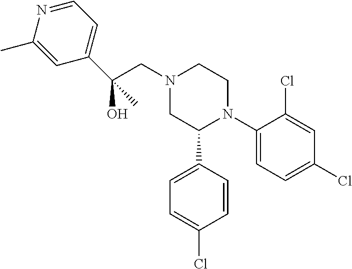 [SMILES: Cc1cc([C@](C)(O)CN2CCN(c3ccc(Cl)cc3Cl)[C@H](c3ccc(Cl)cc3)C2)ccn1] | 442.3 | 2.52 |
| 1015 | 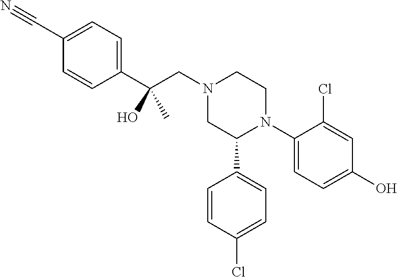 [SMILES: C[C@@](O)(CN1CCN(c2ccc(O)cc2Cl)[C@H](c2ccc(Cl)cc2)C1)c1ccc(C#N)cc1] | 482.3 | 3.15 |
| 1016 | 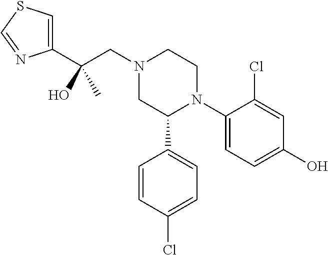 | 464.3 | 2.87 |
| 1017 | 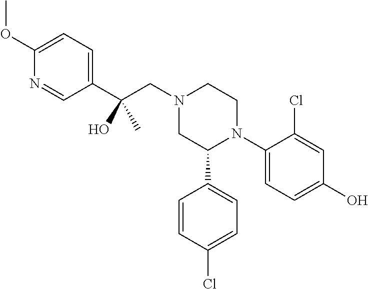 [SMILES: COc1ccc([C@](C)(O)CN2CCN(c3ccc(O)cc3Cl)[C@H](c3ccc(Cl)cc3)C2)cn1] | 488.3 | 2.81 |
| 1018 | 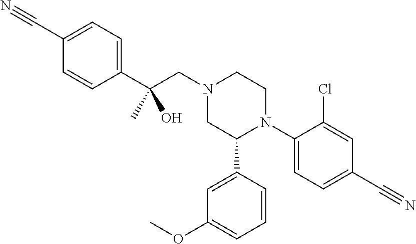 [SMILES: COc1cccc([C@@H]2CN(C[C@@](C)(O)c3ccc(C#N)cc3)CCN2c2ccc(C#N)cc2Cl)c1] | 487.3 | 3.42 |
| 1019 | 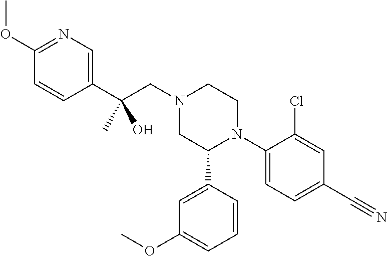 [SMILES: COc1cccc([C@@H]2CN(C[C@@](C)(O)c3ccc(OC)nc3)CCN2c2ccc(C#N)cc2Cl)c1] | 493.3 | 3.04 |
| 1020 | 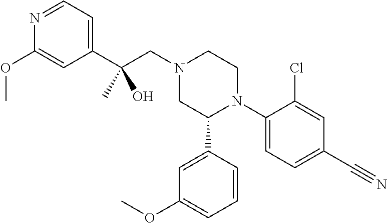 [SMILES: COc1cccc([C@@H]2CN(C[C@@](C)(O)c3ccnc(OC)c3)CCN2c2ccc(C#N)cc2Cl)c1] | 493.3 | 2.96 |
| 1021 | 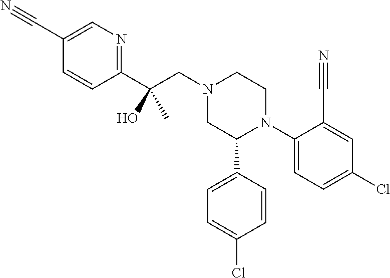 [SMILES: C[C@@](O)(CN1CCN(c2ccc(Cl)cc2C#N)[C@H](c2ccc(Cl)cc2)C1)c1ccc(C#N)cn1] | 492.3 | 3.73 |
| 1022 | 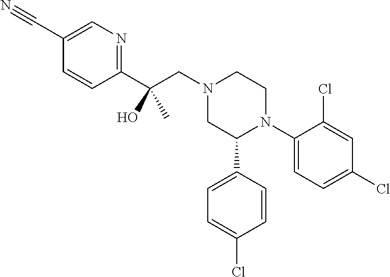 [SMILES: C[C@@](O)(CN1CCN(c2ccc(Cl)cc2Cl)[C@H](c2ccc(Cl)cc2)C1)c1ccc(C#N)cn1] | 503.3 | 4.03 |
| 1023 | 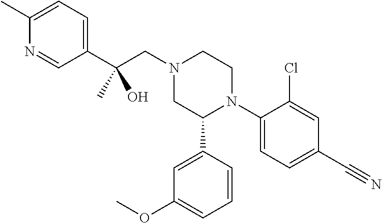 [SMILES: COc1cccc([C@@H]2CN(C[C@@](C)(O)c3ccc(C)nc3)CCN2c2ccc(C#N)cc2Cl)c1] | 477.3 | 2.52 |
| 1024 | 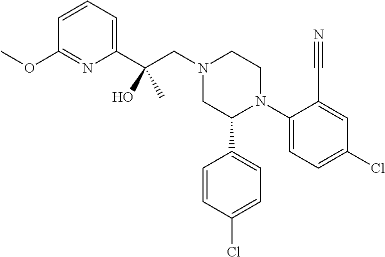 [SMILES: COc1cccc([C@](C)(O)CN2CCN(c3ccc(Cl)cc3C#N)[C@H](c3ccc(Cl)cc3)C2)n1] | 497.3 | 3.69 |
| 1025 | 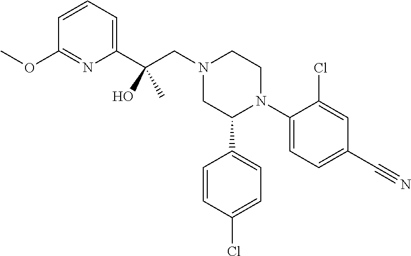 [SMILES: COc1cccc([C@](C)(O)CN2CCN(c3ccc(C#N)cc3Cl)[C@H](c3ccc(Cl)cc3)C2)n1] | 497.3 | 3.61 |
| 1026 | 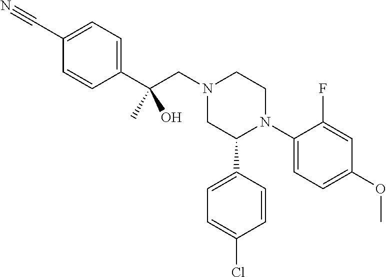 [SMILES: COc1ccc(N2CCN(C[C@@](C)(O)c3ccc(C#N)cc3)C[C@H]2c2ccc(Cl)cc2)c(F)c1] | 480.3 | 3.35 |
| 1027 | 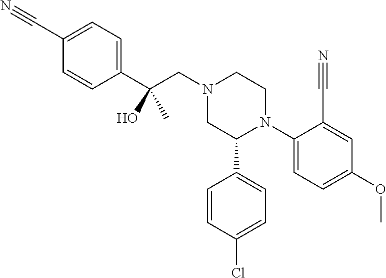 [SMILES: COc1ccc(N2CCN(C[C@@](C)(O)c3ccc(C#N)cc3)C[C@H]2c2ccc(Cl)cc2)c(C#N)c1] | 487.3 | 3.57 |
| 1028 | 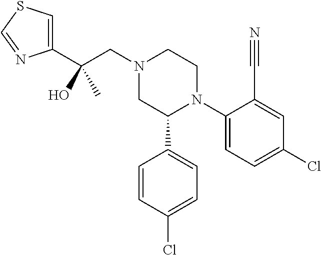 [SMILES: C[C@@](O)(CN1CCN(c2ccc(Cl)cc2C#N)[C@H](c2ccc(Cl)cc2)C1)c1cscn1] | 473.3 | 2.97 |
| 1029 | 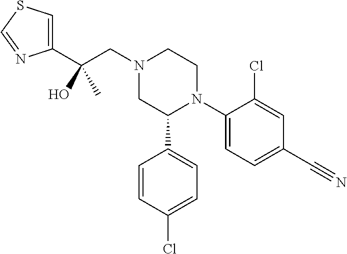 [SMILES: C[C@@](O)(CN1CCN(c2ccc(C#N)cc2Cl)[C@H](c2ccc(Cl)cc2)C1)c1cscn1] | 473.3 | 2.93 |
| 1030 | 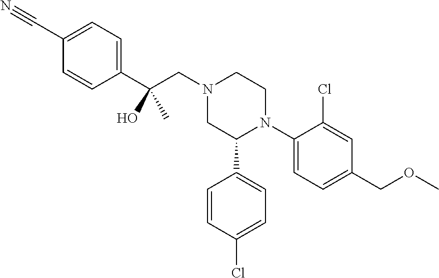 [SMILES: COCc1ccc(N2CCN(C[C@@](C)(O)c3ccc(C#N)cc3)C[C@H]2c2ccc(Cl)cc2)c(Cl)c1] | 510.3 | 3.18 |
| 1032 | 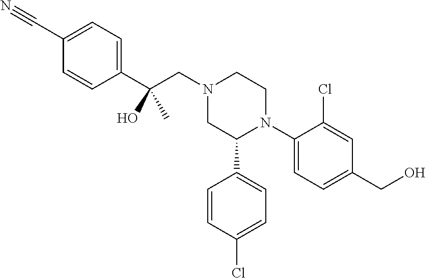 [SMILES: C[C@@](O)(CN1CCN(c2ccc(CO)cc2Cl)[C@H](c2ccc(Cl)cc2)C1)c1ccc(C#N)cc1] | 496.3 | 3.27 |
| 1033 | 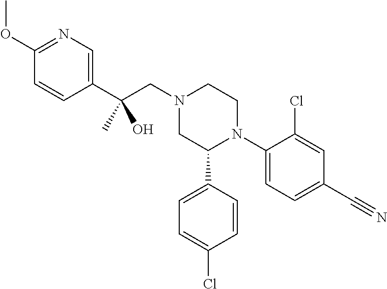 [SMILES: COc1ccc([C@](C)(O)CN2CCN(c3ccc(C#N)cc3Cl)[C@H](c3ccc(Cl)cc3)C2)cn1] | 497.3 | 3.37 |
| 1034 | 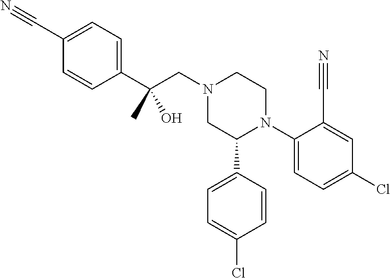 [SMILES: C[C@](O)(CN1CCN(c2ccc(Cl)cc2C#N)[C@H](c2ccc(Cl)cc2)C1)c1ccc(C#N)cc1] | 491.3 | 3.68 |
| 1035d | 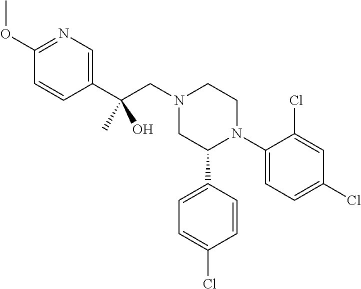 [SMILES: COc1ccc([C@](C)(O)CN2CCN(c3ccc(Cl)cc3Cl)[C@H](c3ccc(Cl)cc3)C2)cn1] | 508.3 | 3.85 |
| 1035e | 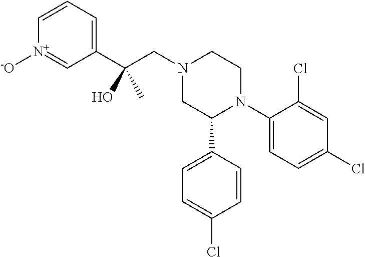 | 492.3 | 2.83 |
| 1035f | 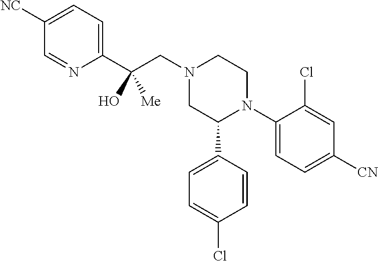 [SMILES: C[C@@](O)(CN1CCN(c2ccc(C#N)cc2Cl)[C@H](c2ccc(Cl)cc2)C1)c1ccc(C#N)cn1] | 492.3 | 3.15 |
| 1035 | 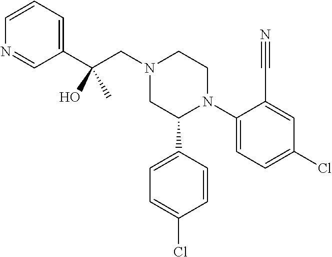 [SMILES: C[C@@](O)(CN1CCN(c2ccc(Cl)cc2C#N)[C@H](c2ccc(Cl)cc2)C1)c1cccnc1] | 467.3 | 2.75 |
| 1035g | 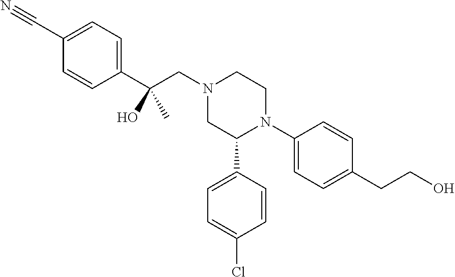 [SMILES: C[C@@](O)(CN1CCN(c2ccc(CCO)cc2)[C@H](c2ccc(Cl)cc2)C1)c1ccc(C#N)cc1] | 476.3 | 3.04 |
| 1035g | 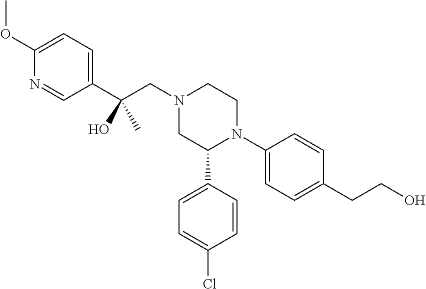 [SMILES: COc1ccc([C@](C)(O)CN2CCN(c3ccc(CCO)cc3)[C@H](c3ccc(Cl)cc3)C2)cn1] | 482.3 | 2.91 |
| 1035h | 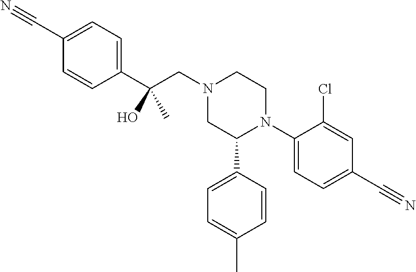 [SMILES: Cc1ccc([C@@H]2CN(C[C@@](C)(O)c3ccc(C#N)cc3)CCN2c2ccc(C#N)cc2Cl)cc1] | 471.3 | 3.58 |
| 1035a | 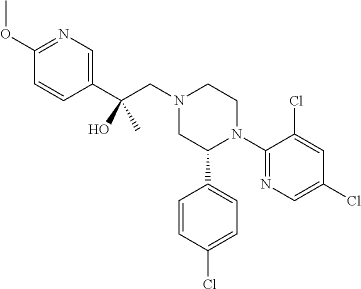 [SMILES: COc1ccc([C@](C)(O)CN2CCN(c3ncc(Cl)cc3Cl)[C@H](c3ccc(Cl)cc3)C2)cn1] | 509.3 | 3.42 |
| 1035c | 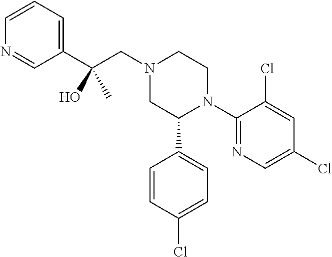 [SMILES: C[C@@](O)(CN1CCN(c2ncc(Cl)cc2Cl)[C@H](c2ccc(Cl)cc2)C1)c1cccnc1] | 477.3 | 2.71 |
| 1035b | 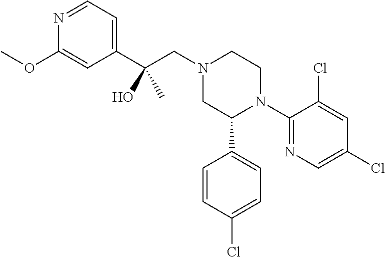 [SMILES: COc1cc([C@](C)(O)CN2CCN(c3ncc(Cl)cc3Cl)[C@H](c3ccc(Cl)cc3)C2)ccn1] | 507.3 | 3.57 |
| 1035i | 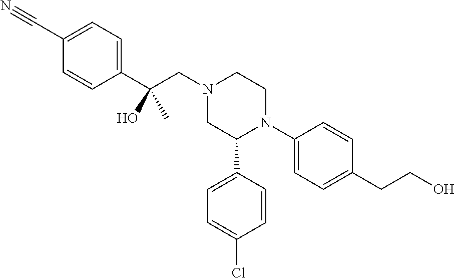 [SMILES: C[C@@](O)(CN1CCN(c2ccc(CCO)cc2)[C@H](c2ccc(Cl)cc2)C1)c1ccc(C#N)cc1] | 476.3 | 3.04 |
| 1036 | 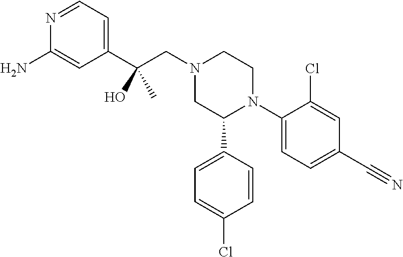 [SMILES: C[C@@](O)(CN1CCN(c2ccc(C#N)cc2Cl)[C@H](c2ccc(Cl)cc2)C1)c1ccnc(N)c1] | 482.3 | 2.63 |
| 1037 | 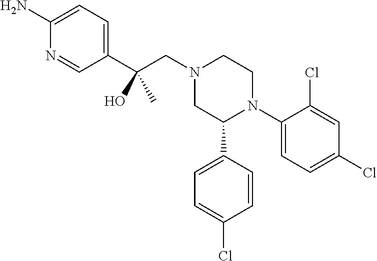 [SMILES: C[C@@](O)(CN1CCN(c2ccc(Cl)cc2Cl)[C@H](c2ccc(Cl)cc2)C1)c1ccc(N)nc1] | 491.3 | 2.65 |
| 1038 | 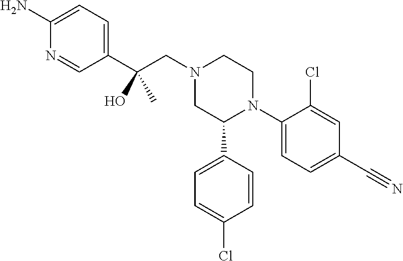 [SMILES: C[C@@](O)(CN1CCN(c2ccc(C#N)cc2Cl)[C@H](c2ccc(Cl)cc2)C1)c1ccc(N)nc1] | 482.3 | 2.43 |
| 1039 | 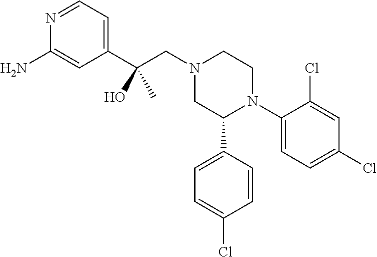 [SMILES: C[C@@](O)(CN1CCN(c2ccc(Cl)cc2Cl)[C@H](c2ccc(Cl)cc2)C1)c1ccnc(N)c1] | 493.3 | 2.85 |
| 1040 | 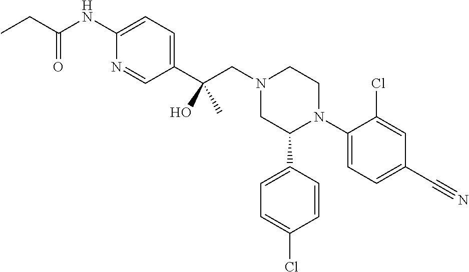 [SMILES: CCC(=O)Nc1ccc([C@](C)(O)CN2CCN(c3ccc(C#N)cc3Cl)[C@H](c3ccc(Cl)cc3)C2)cn1] | 583.3 | 3.28 |
| 1041 | 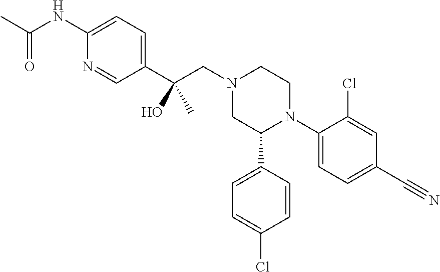 [SMILES: CC(=O)Nc1ccc([C@](C)(O)CN2CCN(c3ccc(C#N)cc3Cl)[C@H](c3ccc(Cl)cc3)C2)cn1] | 524.3 | 3.02 |
| 1042 | 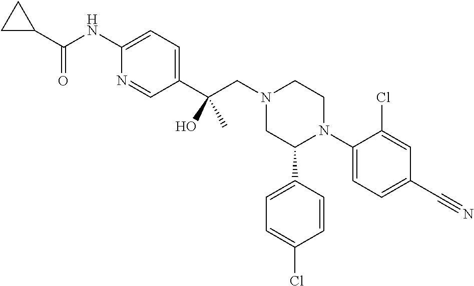 [SMILES: C[C@@](O)(CN1CCN(c2ccc(C#N)cc2Cl)[C@H](c2ccc(Cl)cc2)C1)c1ccc(NC(=O)C2CC2)nc1] | 550.3 | 3.32 |
| 1043 | 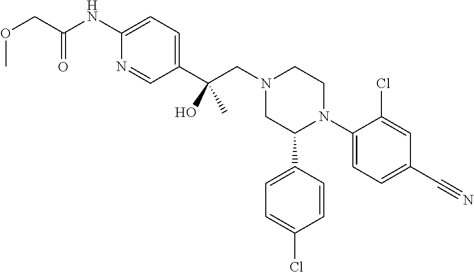 [SMILES: COCC(=O)Nc1ccc([C@](C)(O)CN2CCN(c3ccc(C#N)cc3Cl)[C@H](c3ccc(Cl)cc3)C2)cn1] | 554.3 | 3.23 |
| 1044 | 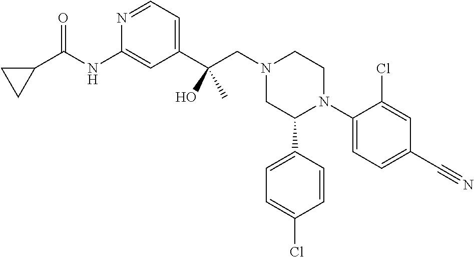 [SMILES: C[C@@](O)(CN1CCN(c2ccc(C#N)cc2Cl)[C@H](c2ccc(Cl)cc2)C1)c1ccnc(NC(=O)C2CC2)c1] | 550.3 | 3.15 |
| 1045 | 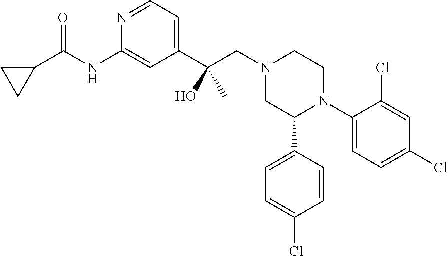 [SMILES: C[C@@](O)(CN1CCN(c2ccc(Cl)cc2Cl)[C@H](c2ccc(Cl)cc2)C1)c1ccnc(NC(=O)C2CC2)c1] | 561.3 | 3.47 |
| 1046 | 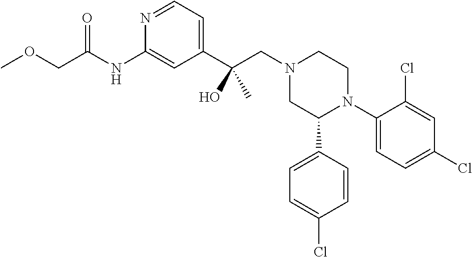 [SMILES: COCC(=O)Nc1cc([C@](C)(O)CN2CCN(c3ccc(Cl)cc3Cl)[C@H](c3ccc(Cl)cc3)C2)ccn1] | 563.3 | 3.42 |
| 1048 | 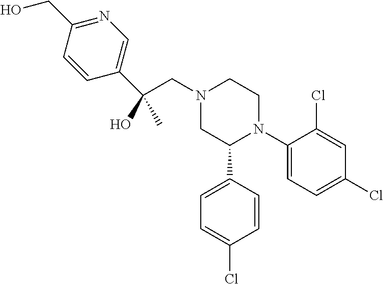 [SMILES: C[C@@](O)(CN1CCN(c2ccc(Cl)cc2Cl)[C@H](c2ccc(Cl)cc2)C1)c1ccc(CO)nc1] | 508.3 | 2.72 |
| 1049 | 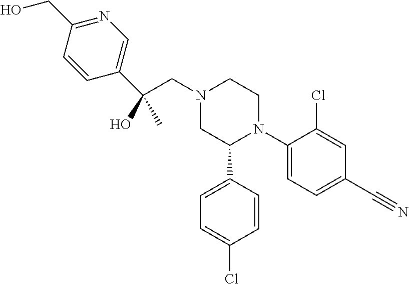 [SMILES: C[C@@](O)(CN1CCN(c2ccc(C#N)cc2Cl)[C@H](c2ccc(Cl)cc2)C1)c1ccc(CO)nc1] | 497.3 | 2.44 |
| 1050 | 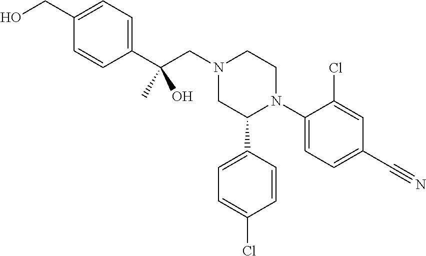 [SMILES: C[C@@](O)(CN1CCN(c2ccc(C#N)cc2Cl)[C@H](c2ccc(Cl)cc2)C1)c1ccc(CO)cc1] | 496.3 | 3.11 |
| 1051 | 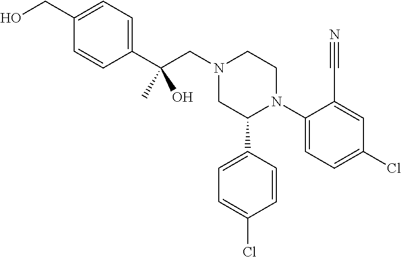 [SMILES: C[C@@](O)(CN1CCN(c2ccc(Cl)cc2C#N)[C@H](c2ccc(Cl)cc2)C1)c1ccc(CO)cc1] | 496.3 | 3.34 |
| 1052 | 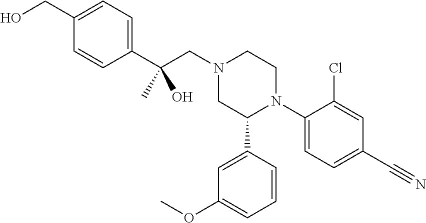 [SMILES: COc1cccc([C@@H]2CN(C[C@@](C)(O)c3ccc(CO)cc3)CCN2c2ccc(C#N)cc2Cl)c1] | 492.3 | 3.03 |
| 1053 | 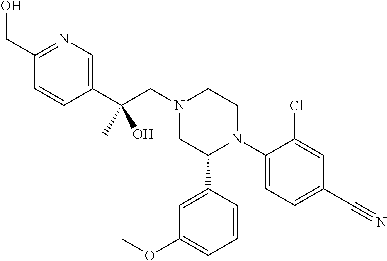 [SMILES: COc1cccc([C@@H]2CN(C[C@@](C)(O)c3ccc(CO)nc3)CCN2c2ccc(C#N)cc2Cl)c1] | 493.3 | 2.58 |
| 1054 | 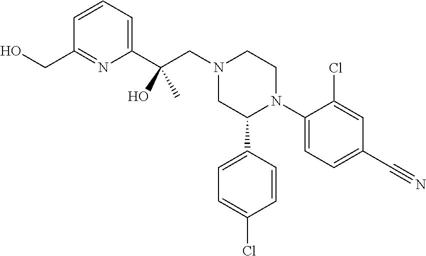 [SMILES: C[C@@](O)(CN1CCN(c2ccc(C#N)cc2Cl)[C@H](c2ccc(Cl)cc2)C1)c1cccc(CO)n1] | 497.3 | 3.16 |
| 1055 | 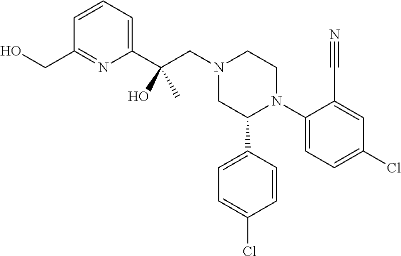 [SMILES: C[C@@](O)(CN1CCN(c2ccc(Cl)cc2C#N)[C@H](c2ccc(Cl)cc2)C1)c1cccc(CO)n1] | 497.3 | 3.72 |
| 1056 | 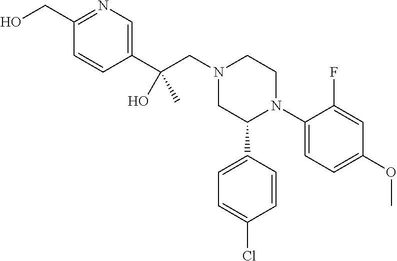 [SMILES: COc1ccc(N2CCN(C[C@@](C)(O)c3ccc(CO)nc3)C[C@H]2c2ccc(Cl)cc2)c(F)c1] | 486.3 | 2.51 |
| 1057 | 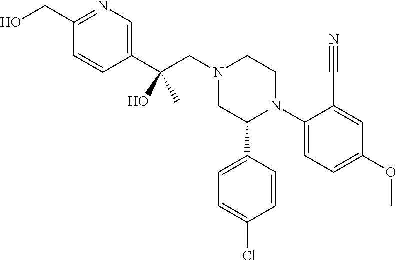 [SMILES: COc1ccc(N2CCN(C[C@@](C)(O)c3ccc(CO)nc3)C[C@H]2c2ccc(Cl)cc2)c(C#N)c1] | 493.3 | 2.56 |
| 1058 | 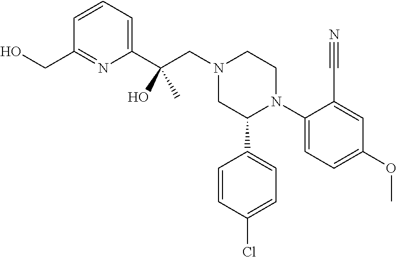 [SMILES: COc1ccc(N2CCN(C[C@@](C)(O)c3cccc(CO)n3)C[C@H]2c2ccc(Cl)cc2)c(C#N)c1] | 493.3 | 3.13 |
| 1059 | 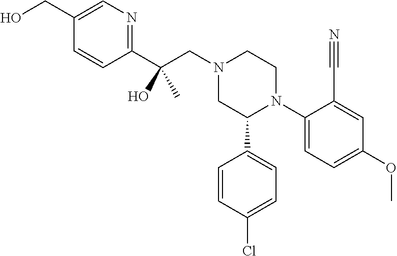 [SMILES: COc1ccc(N2CCN(C[C@@](C)(O)c3ccc(CO)cn3)C[C@H]2c2ccc(Cl)cc2)c(C#N)c1] | 493.3 | 3.08 |
| 1060 | 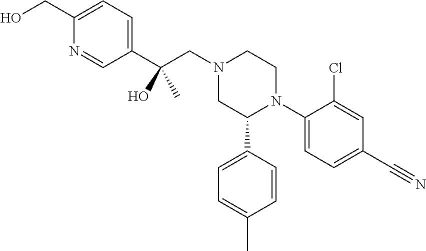 [SMILES: Cc1ccc([C@@H]2CN(C[C@@](C)(O)c3ccc(CO)nc3)CCN2c2ccc(C#N)cc2Cl)cc1] | 477.3 | 2.40 |
| 1061 | 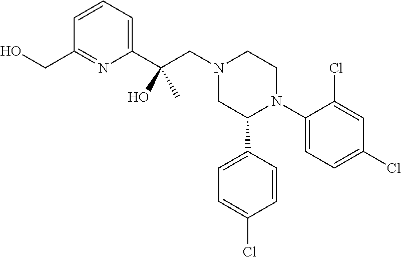 [SMILES: C[C@@](O)(CN1CCN(c2ccc(Cl)cc2Cl)[C@H](c2ccc(Cl)cc2)C1)c1cccc(CO)n1] | 508.3 | 3.40 |
| 1062a | 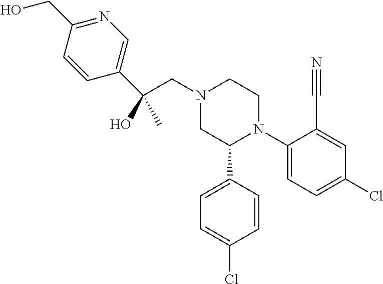 [SMILES: C[C@@](O)(CN1CCN(c2ccc(Cl)cc2C#N)[C@H](c2ccc(Cl)cc2)C1)c1ccc(CO)nc1] | 497.3 | 2.53 |
| 1062c | 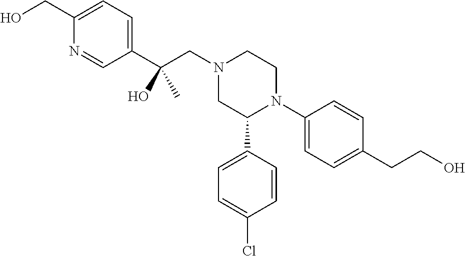 [SMILES: C[C@@](O)(CN1CCN(c2ccc(CCO)cc2)[C@H](c2ccc(Cl)cc2)C1)c1ccc(CO)nc1] | 482.3 | 2.35 |
| 1062b | 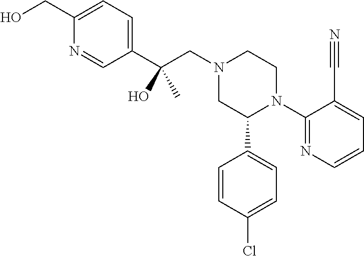 [SMILES: C[C@@](O)(CN1CCN(c2ncccc2C#N)[C@H](c2ccc(Cl)cc2)C1)c1ccc(CO)nc1] | 464.3 | 2.25 |
| 1063 | 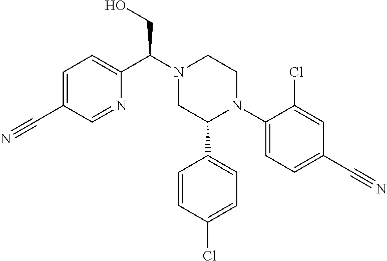 [SMILES: N#Cc1ccc([C@@H](CO)N2CCN(c3ccc(C#N)cc3Cl)[C@H](c3ccc(Cl)cc3)C2)nc1] | 478.3 | 3.39 |
| 1064 | 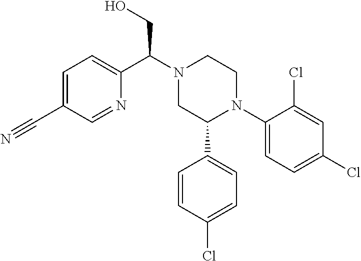 [SMILES: N#Cc1ccc([C@@H](CO)N2CCN(c3ccc(Cl)cc3Cl)[C@H](c3ccc(Cl)cc3)C2)nc1] | 489.3 | 3.86 |
| 1065 | 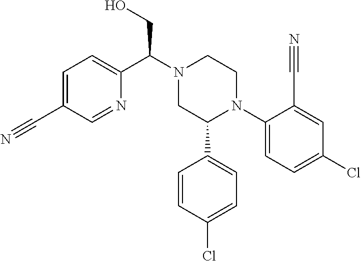 [SMILES: N#Cc1ccc([C@@H](CO)N2CCN(c3ccc(Cl)cc3C#N)[C@H](c3ccc(Cl)cc3)C2)nc1] | 478.3 | 3.55 |
| 1068 | 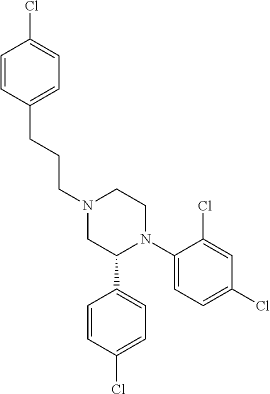 [SMILES: Clc1ccc(CCCN2CCN(c3ccc(Cl)cc3Cl)[C@H](c3ccc(Cl)cc3)C2)cc1] | 495.3 | 4.17 |
| 1069 | 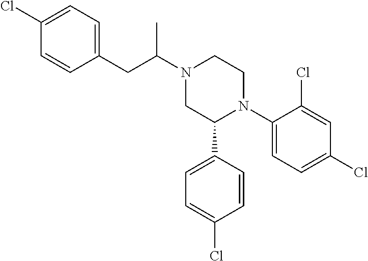 [SMILES: CC(Cc1ccc(Cl)cc1)N1CCN(c2ccc(Cl)cc2Cl)[C@H](c2ccc(Cl)cc2)C1] | 495.5 | 4.15 |
| 1070 | 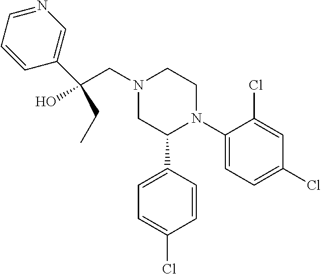 [SMILES: CC[C@](O)(CN1CCN(c2ccc(Cl)cc2Cl)[C@H](c2ccc(Cl)cc2)C1)c1cccnc1] | 492.3 | 2.65 |
| 1071 | 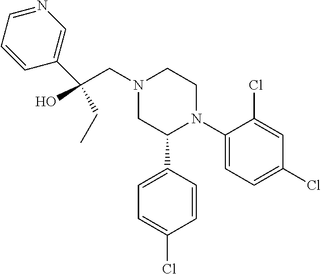 [SMILES: CC[C@@](O)(CN1CCN(c2ccc(Cl)cc2Cl)[C@H](c2ccc(Cl)cc2)C1)c1cccnc1] | 492.3 | 2.65 |
| 1072 | 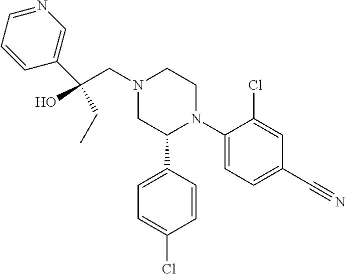 [SMILES: CC[C@@](O)(CN1CCN(c2ccc(C#N)cc2Cl)[C@H](c2ccc(Cl)cc2)C1)c1cccnc1] | 481.3 | 2.42 |
| 1073 | 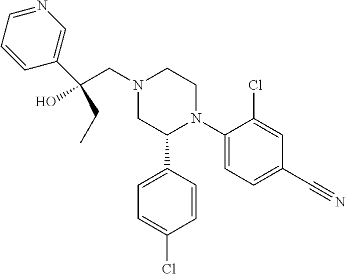 [SMILES: CC[C@](O)(CN1CCN(c2ccc(C#N)cc2Cl)[C@H](c2ccc(Cl)cc2)C1)c1cccnc1] | 481.3 | 2.44 |
| 1074 | 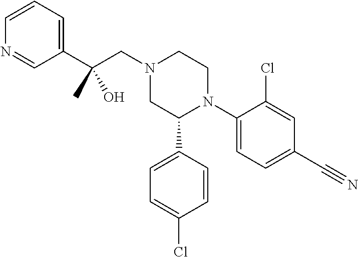 [SMILES: C[C@](O)(CN1CCN(c2ccc(C#N)cc2Cl)[C@H](c2ccc(Cl)cc2)C1)c1cccnc1] | 467.3 | 2.36 |
| 1075 | 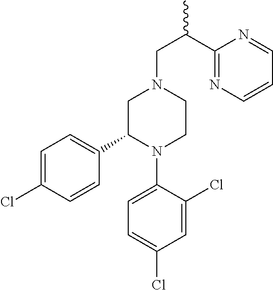 [SMILES: CC(CN1CCN(c2ccc(Cl)cc2Cl)[C@H](c2ccc(Cl)cc2)C1)c1ncccn1] | 463.3 | 3.32 |
| 1077 | 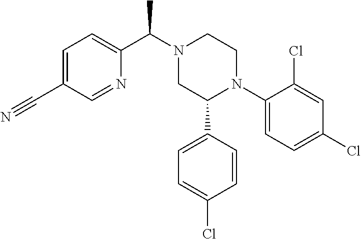 [SMILES: C[C@H](c1ccc(C#N)cn1)N1CCN(c2ccc(Cl)cc2Cl)[C@H](c2ccc(Cl)cc2)C1] | 471.3 | 3.47 |
| 1079 | 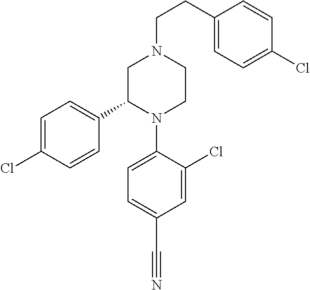 [SMILES: N#Cc1ccc(N2CCN(CCc3ccc(Cl)cc3)C[C@H]2c2ccc(Cl)cc2)c(Cl)c1] | 470.3 | 3.63 |
| 1080 | 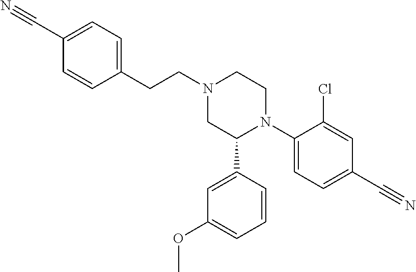 [SMILES: COc1cccc([C@@H]2CN(CCc3ccc(C#N)cc3)CCN2c2ccc(C#N)cc2Cl)c1] | 457.2 | 3.23 |
| 1081 | 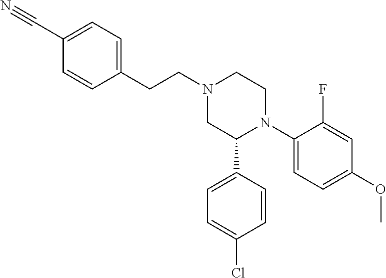 [SMILES: COc1ccc(N2CCN(CCc3ccc(C#N)cc3)C[C@H]2c2ccc(Cl)cc2)c(F)c1] | 450.2 | 3.62 |
| 1082 | 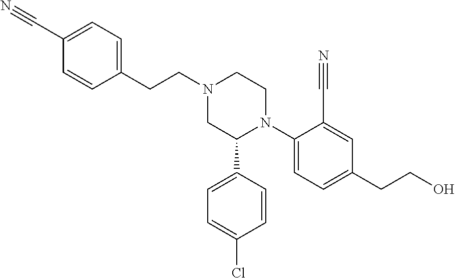 [SMILES: N#Cc1ccc(CCN2CCN(c3ccc(CCO)cc3C#N)[C@H](c3ccc(Cl)cc3)C2)cc1] | 471.3 | 3.06 |
| 1082b | 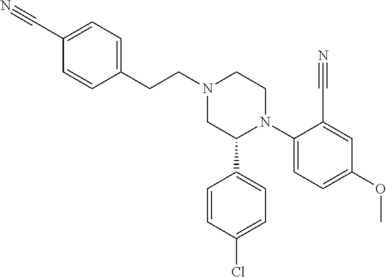 [SMILES: COc1ccc(N2CCN(CCc3ccc(C#N)cc3)C[C@H]2c2ccc(Cl)cc2)c(C#N)c1] | 457.3 | 3.35 |
| 1082c | 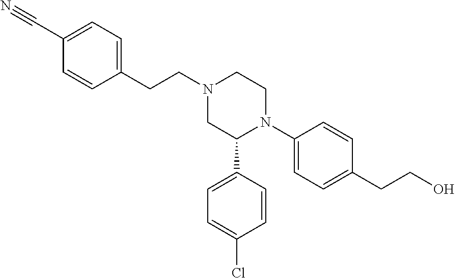 [SMILES: N#Cc1ccc(CCN2CCN(c3ccc(CCO)cc3)[C@H](c3ccc(Cl)cc3)C2)cc1] | 446.2 | 3.28 |
| 1083 | 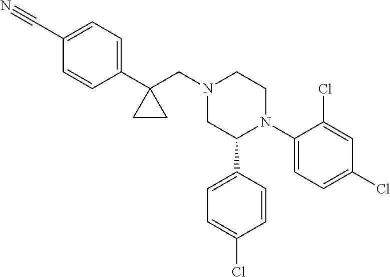 [SMILES: N#Cc1ccc(C2(CN3CCN(c4ccc(Cl)cc4Cl)[C@H](c4ccc(Cl)cc4)C3)CC2)cc1] | 496.3 | 3.83 |
| 1084 | 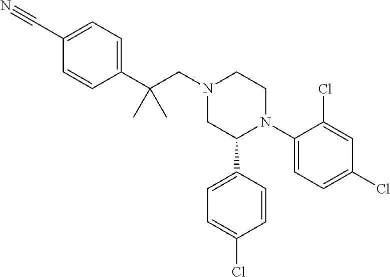 [SMILES: CC(C)(CN1CCN(c2ccc(Cl)cc2Cl)[C@H](c2ccc(Cl)cc2)C1)c1ccc(C#N)cc1] | 500.3 | 3.99 |
| 1085 | 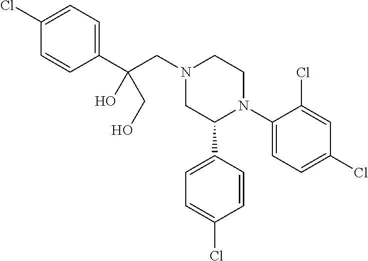 [SMILES: OCC(O)(CN1CCN(c2ccc(Cl)cc2Cl)[C@H](c2ccc(Cl)cc2)C1)c1ccc(Cl)cc1] | 527.3 | 3.58 |
| 1086 | 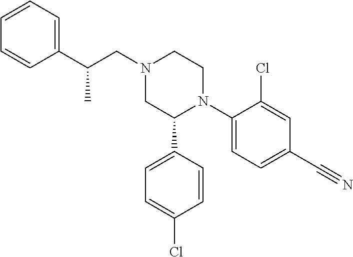 [SMILES: C[C@@H](CN1CCN(c2ccc(C#N)cc2Cl)[C@H](c2ccc(Cl)cc2)C1)c1ccccc1] | 450.2 | 3.59 |
| 1087a | 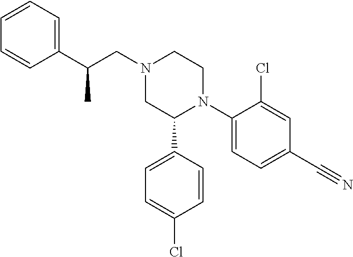 [SMILES: C[C@H](CN1CCN(c2ccc(C#N)cc2Cl)[C@H](c2ccc(Cl)cc2)C1)c1ccccc1] | 450.2 | 3.60 |
| 1088 | 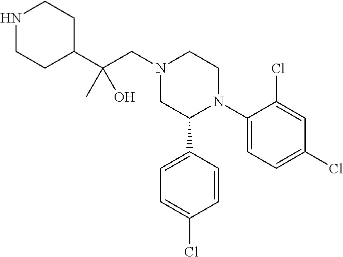 [SMILES: CC(O)(CN1CCN(c2ccc(Cl)cc2Cl)[C@H](c2ccc(Cl)cc2)C1)C1CCNCC1] | 484.3 | 2.69 |
| 1089 | 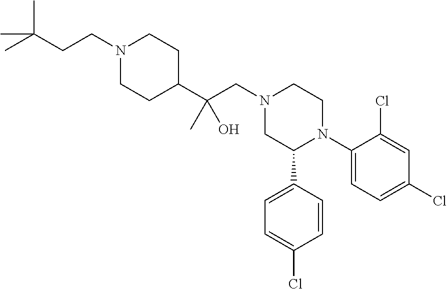 [SMILES: CC(C)(C)CCN1CCC(C(C)(O)CN2CCN(c3ccc(Cl)cc3Cl)[C@H](c3ccc(Cl)cc3)C2)CC1] | 568.3 | 2.93 |
| 1090 | 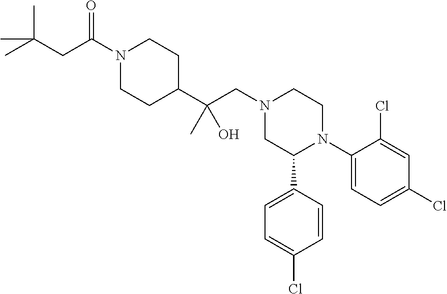 [SMILES: CC(C)(C)CC(=O)N1CCC(C(C)(O)CN2CCN(c3ccc(Cl)cc3Cl)[C@H](c3ccc(Cl)cc3)C2)CC1] | 582.3 | 3.60 |
| 1091 | 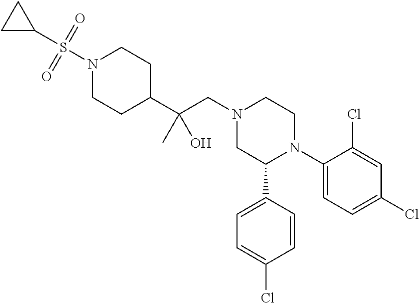 [SMILES: CC(O)(CN1CCN(c2ccc(Cl)cc2Cl)[C@H](c2ccc(Cl)cc2)C1)C1CCN(S(=O)(=O)C2CC2)CC1] | 588.3 | 3.53 |
| 1092 | 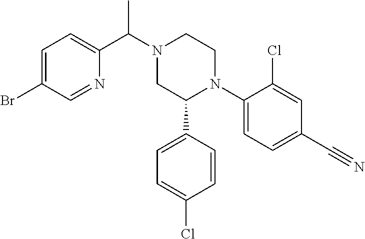 [SMILES: CC(c1ccc(Br)cn1)N1CCN(c2ccc(C#N)cc2Cl)[C@H](c2ccc(Cl)cc2)C1] | 517.3 | 3.57 |
| 1093 | 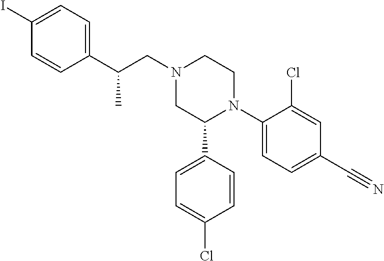 [SMILES: C[C@@H](CN1CCN(c2ccc(C#N)cc2Cl)[C@H](c2ccc(Cl)cc2)C1)c1ccc(I)cc1] | 576.3 | 3.88 |
| 1094 | 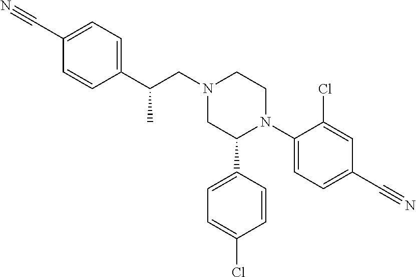 [SMILES: C[C@@H](CN1CCN(c2ccc(C#N)cc2Cl)[C@H](c2ccc(Cl)cc2)C1)c1ccc(C#N)cc1] | 475.3 | 3.55 |
| 1095 | 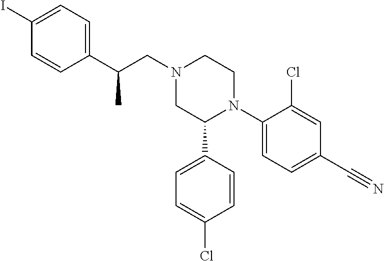 [SMILES: C[C@H](CN1CCN(c2ccc(C#N)cc2Cl)[C@H](c2ccc(Cl)cc2)C1)c1ccc(I)cc1] | 576.3 | 3.91 |
| 1096 | 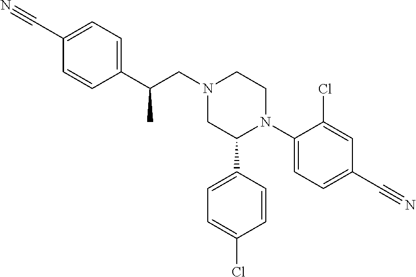 [SMILES: C[C@H](CN1CCN(c2ccc(C#N)cc2Cl)[C@H](c2ccc(Cl)cc2)C1)c1ccc(C#N)cc1] | 475.3 | 3.56 |
| 1097 | 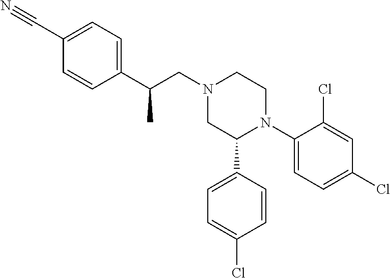 [SMILES: C[C@H](CN1CCN(c2ccc(Cl)cc2Cl)[C@H](c2ccc(Cl)cc2)C1)c1ccc(C#N)cc1] | 486.3 | 4.24 |
| 1098 | 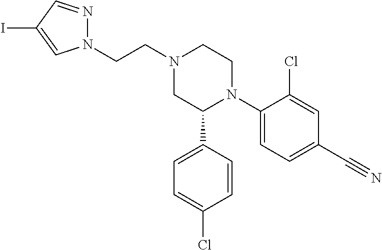 [SMILES: N#Cc1ccc(N2CCN(CCn3cc(I)cn3)C[C@H]2c2ccc(Cl)cc2)c(Cl)c1] | 552.3 | 3.50 |
| 1099 | 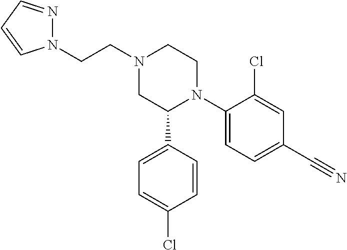 [SMILES: N#Cc1ccc(N2CCN(CCn3cccn3)C[C@H]2c2ccc(Cl)cc2)c(Cl)c1] | 426.2 | 3.32 |
| 1100 | 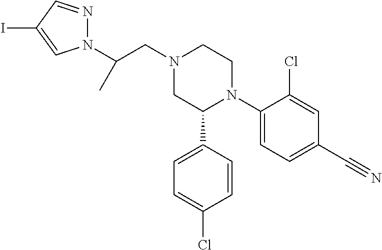 [SMILES: CC(CN1CCN(c2ccc(C#N)cc2Cl)[C@H](c2ccc(Cl)cc2)C1)n1cc(I)cn1] | 566.3 | 3.43 |
| 1101 | 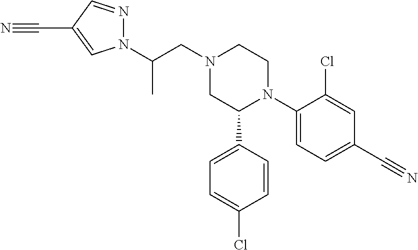 [SMILES: CC(CN1CCN(c2ccc(C#N)cc2Cl)[C@H](c2ccc(Cl)cc2)C1)n1cc(C#N)cn1] | 465.3 | 3.41 |
| 1102 | 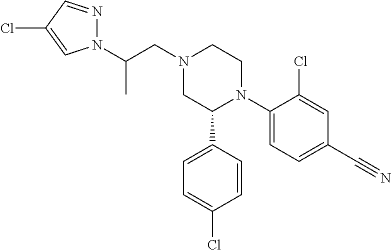 [SMILES: CC(CN1CCN(c2ccc(C#N)cc2Cl)[C@H](c2ccc(Cl)cc2)C1)n1cc(Cl)cn1] | 476.3 | 3.34 |
| 1103 | 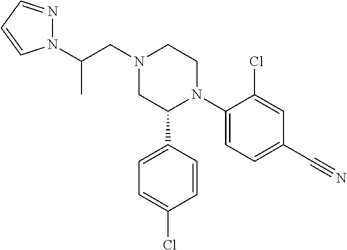 [SMILES: CC(CN1CCN(c2ccc(C#N)cc2Cl)[C@H](c2ccc(Cl)cc2)C1)n1cccn1] | 440.2 | 3.13 |
| 1104a | 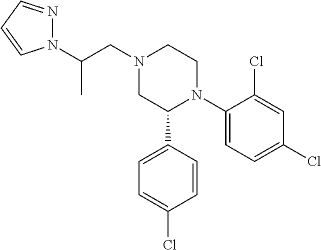 [SMILES: CC(CN1CCN(c2ccc(Cl)cc2Cl)[C@H](c2ccc(Cl)cc2)C1)n1cccn1] | 451.2 | 3.44 |
| 1104b | 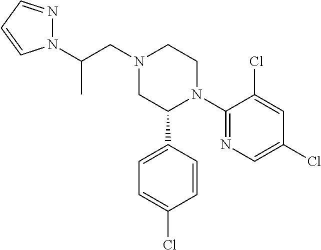 [SMILES: CC(CN1CCN(c2ncc(Cl)cc2Cl)[C@H](c2ccc(Cl)cc2)C1)n1cccn1] | 460.2 | 3.31 |
| 1105 | 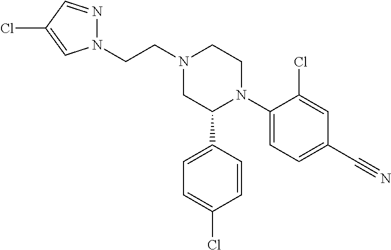 [SMILES: N#Cc1ccc(N2CCN(CCn3cc(Cl)cn3)C[C@H]2c2ccc(Cl)cc2)c(Cl)c1] | 460.3 | 3.18 |
| 1106 | 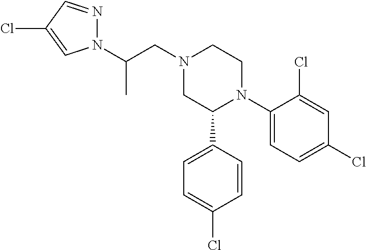 [SMILES: CC(CN1CCN(c2ccc(Cl)cc2Cl)[C@H](c2ccc(Cl)cc2)C1)n1cc(Cl)cn1] | 485.3 | 3.59 |
| 1107 | 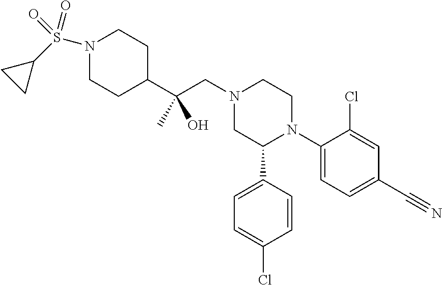 [SMILES: C[C@@](O)(CN1CCN(c2ccc(C#N)cc2Cl)[C@H](c2ccc(Cl)cc2)C1)C1CCN(S(=O)(=O)C2CC2)CC1] | 444.2 | 2.68 |
| 1108 | 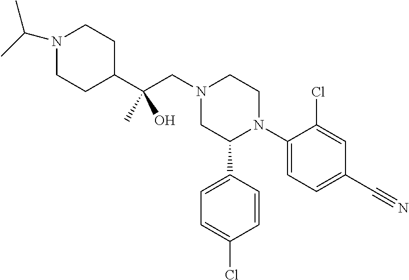 [SMILES: CC(C)N1CCC([C@](C)(O)CN2CCN(c3ccc(C#N)cc3Cl)[C@H](c3ccc(Cl)cc3)C2)CC1] | 515.3 | 2.32 |
| 1109 | 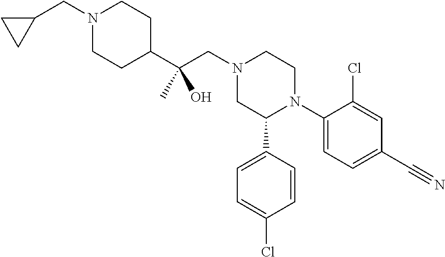 [SMILES: C[C@@](O)(CN1CCN(c2ccc(C#N)cc2Cl)[C@H](c2ccc(Cl)cc2)C1)C1CCN(CC2CC2)CC1] | 527.3 | 2.39 |
| 1110 | 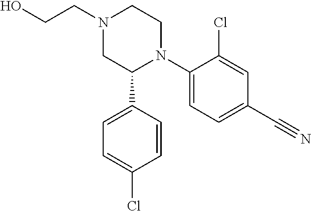 [SMILES: N#Cc1ccc(N2CCN(CCO)C[C@H]2c2ccc(Cl)cc2)c(Cl)c1] | 376.2 | 2.83 |
| 1111 | 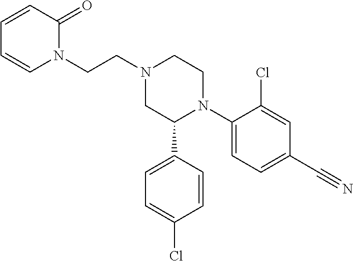 [SMILES: N#Cc1ccc(N2CCN(CCn3ccccc3=O)C[C@H]2c2ccc(Cl)cc2)c(Cl)c1] | 453.2 | 2.81 |
| 1112 | 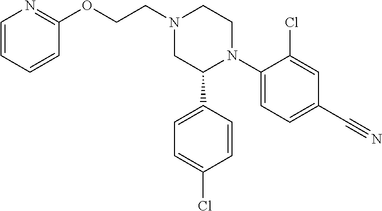 [SMILES: N#Cc1ccc(N2CCN(CCOc3ccccn3)C[C@H]2c2ccc(Cl)cc2)c(Cl)c1] | 358.2 | 3.08 |
| 1113 | 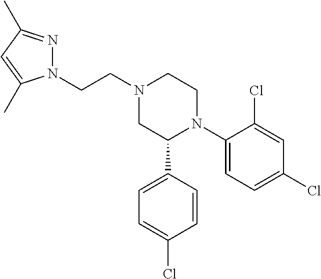 [SMILES: Cc1cc(C)n(CCN2CCN(c3ccc(Cl)cc3Cl)[C@H](c3ccc(Cl)cc3)C2)n1] | 465.3 | 3.65 |
| 1114 | 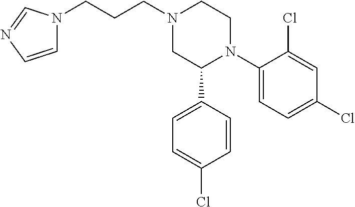 [SMILES: Clc1ccc([C@@H]2CN(CCCn3ccnc3)CCN2c2ccc(Cl)cc2Cl)cc1] | 451.2 | 2.80 |
| 1115 | 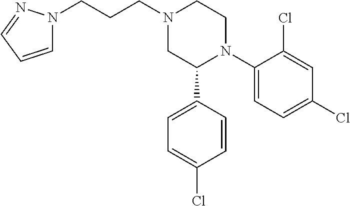 [SMILES: Clc1ccc([C@@H]2CN(CCCn3cccn3)CCN2c2ccc(Cl)cc2Cl)cc1] | 451.2 | 3.46 |
| 1116 | 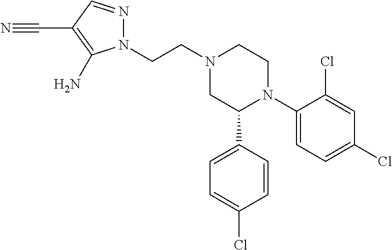 [SMILES: N#Cc1cnn(CCN2CCN(c3ccc(Cl)cc3Cl)[C@H](c3ccc(Cl)cc3)C2)c1N] | 477.3 | 3.50 |
| 1117 | 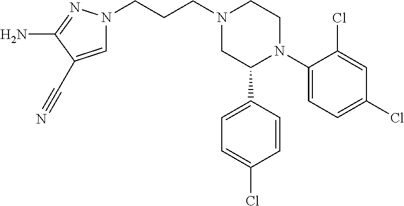 [SMILES: N#Cc1cn(CCCN2CCN(c3ccc(Cl)cc3Cl)[C@H](c3ccc(Cl)cc3)C2)nc1N] | 489.3 | 3.38 |
| 1118 | 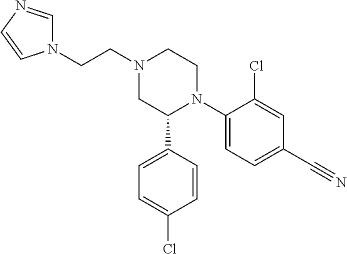 [SMILES: N#Cc1ccc(N2CCN(CCn3ccnc3)C[C@H]2c2ccc(Cl)cc2)c(Cl)c1] | 426.2 | 2.46 |
| 1119 | 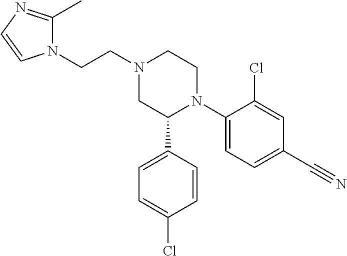 [SMILES: Cc1nccn1CCN1CCN(c2ccc(C#N)cc2Cl)[C@H](c2ccc(Cl)cc2)C1] | 440.2 | 2.47 |
| 1120 | 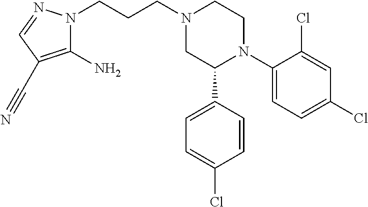 [SMILES: N#Cc1cnn(CCCN2CCN(c3ccc(Cl)cc3Cl)[C@H](c3ccc(Cl)cc3)C2)c1N] | 489.3 | 3.42 |
| 1121 | 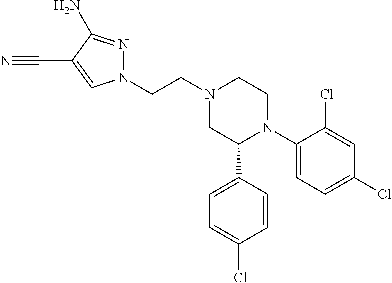 [SMILES: N#Cc1cn(CCN2CCN(c3ccc(Cl)cc3Cl)[C@H](c3ccc(Cl)cc3)C2)nc1N] | 477.3 | 3.41 |
| 1122 | 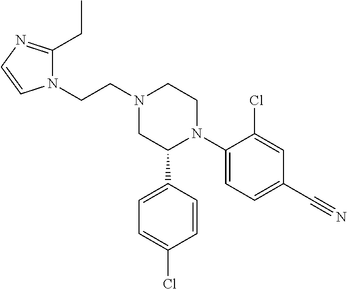 [SMILES: CCc1nccn1CCN1CCN(c2ccc(C#N)cc2Cl)[C@H](c2ccc(Cl)cc2)C1] | 454.2 | 2.88 |
| 1123 | 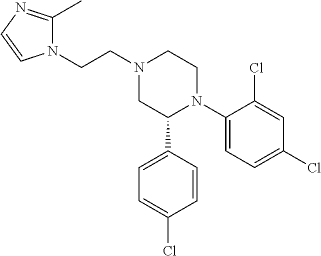 [SMILES: Cc1nccn1CCN1CCN(c2ccc(Cl)cc2Cl)[C@H](c2ccc(Cl)cc2)C1] | 451.2 | 2.95 |
| 1124 | 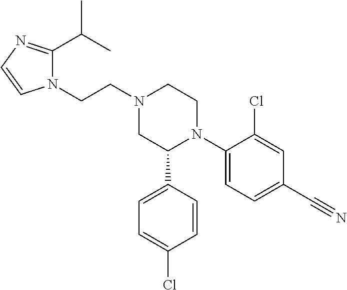 [SMILES: CC(C)c1nccn1CCN1CCN(c2ccc(C#N)cc2Cl)[C@H](c2ccc(Cl)cc2)C1] | 468.3 | 2.70 |
| 1125 | 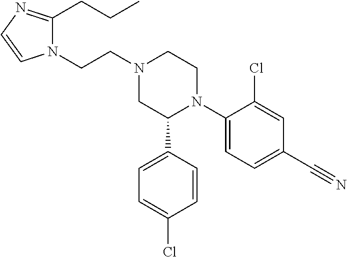 [SMILES: CCCc1nccn1CCN1CCN(c2ccc(C#N)cc2Cl)[C@H](c2ccc(Cl)cc2)C1] | 468.3 | 2.70 |
| 1126 | 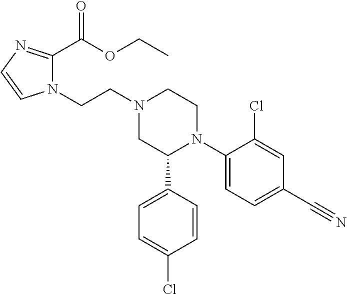 [SMILES: CCOC(=O)c1nccn1CCN1CCN(c2ccc(C#N)cc2Cl)[C@H](c2ccc(Cl)cc2)C1] | 498.3 | 2.88 |
| 1127 | 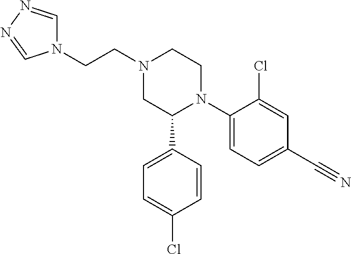 [SMILES: N#Cc1ccc(N2CCN(CCn3cnnc3)C[C@H]2c2ccc(Cl)cc2)c(Cl)c1] | 427.2 | 2.66509.3 |
| 1128 | 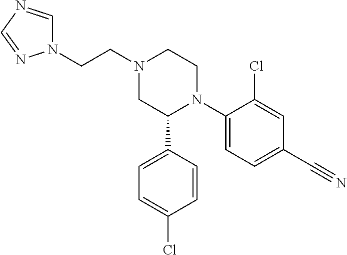 [SMILES: N#Cc1ccc(N2CCN(CCn3cncn3)C[C@H]2c2ccc(Cl)cc2)c(Cl)c1] | 427.2 | 2.71 |
| 1129 | 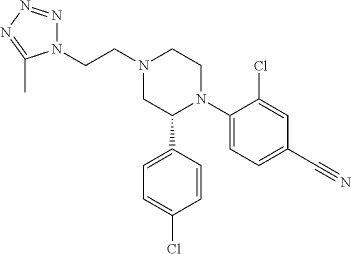 [SMILES: Cc1nnnn1CCN1CCN(c2ccc(C#N)cc2Cl)[C@H](c2ccc(Cl)cc2)C1] | 442.2 | 3.24 |
| 1130 | 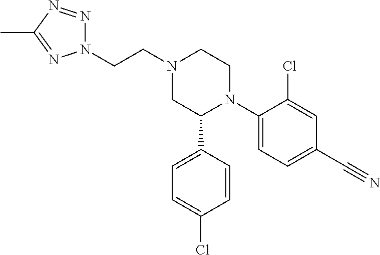 [SMILES: Cc1nnn(CCN2CCN(c3ccc(C#N)cc3Cl)[C@H](c3ccc(Cl)cc3)C2)n1] | 442.2 | 3.36 |
| 1131 | 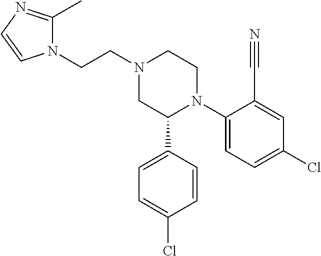 [SMILES: Cc1nccn1CCN1CCN(c2ccc(Cl)cc2C#N)[C@H](c2ccc(Cl)cc2)C1] | 440.2 | 2.76 |
| 1131 | 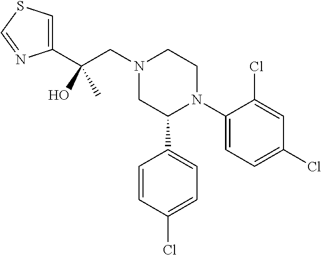 [SMILES: C[C@@](O)(CN1CCN(c2ccc(Cl)cc2Cl)[C@H](c2ccc(Cl)cc2)C1)c1cscn1] | 482.3 | 3.20 |
| 1132 | 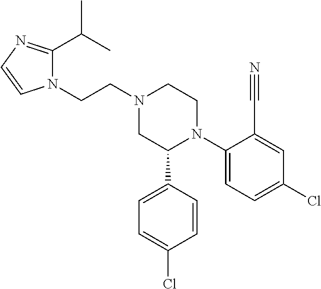 [SMILES: CC(C)c1nccn1CCN1CCN(c2ccc(Cl)cc2C#N)[C@H](c2ccc(Cl)cc2)C1] | 468.3 | 2.92 |
| 1133 | 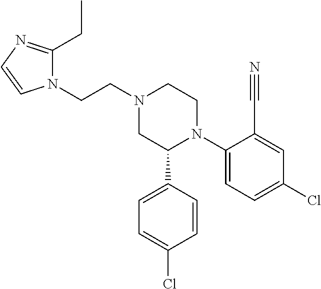 [SMILES: CCc1nccn1CCN1CCN(c2ccc(Cl)cc2C#N)[C@H](c2ccc(Cl)cc2)C1] | 454.2 | 2.83 |
| 1134 | 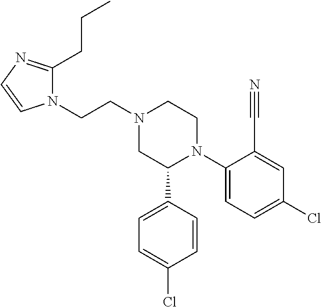 [SMILES: CCCc1nccn1CCN1CCN(c2ccc(Cl)cc2C#N)[C@H](c2ccc(Cl)cc2)C1] | 468.3 | 2.95 |
| 1135 | 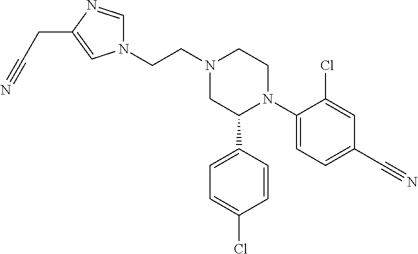 [SMILES: N#CCc1cn(CCN2CCN(c3ccc(C#N)cc3Cl)[C@H](c3ccc(Cl)cc3)C2)cn1] | 465.3 | 2.90 |
| 1136 | 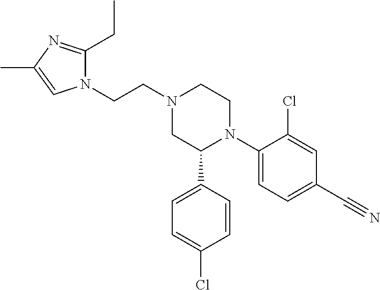 [SMILES: CCc1nc(C)cn1CCN1CCN(c2ccc(C#N)cc2Cl)[C@H](c2ccc(Cl)cc2)C1] | 468.3 | 2.79 |
| 1137 | 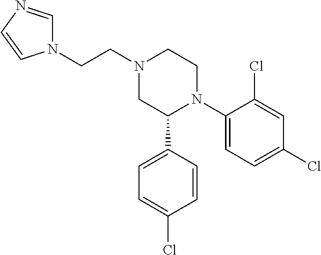 [SMILES: Clc1ccc([C@@H]2CN(CCn3ccnc3)CCN2c2ccc(Cl)cc2Cl)cc1] | 435.2 | 2.87 |
| 1138 | 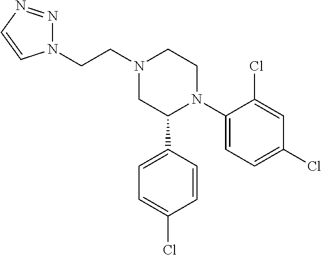 [SMILES: Clc1ccc([C@@H]2CN(CCn3ccnn3)CCN2c2ccc(Cl)cc2Cl)cc1] | 438.2 | 3.41 |
| 1139 | 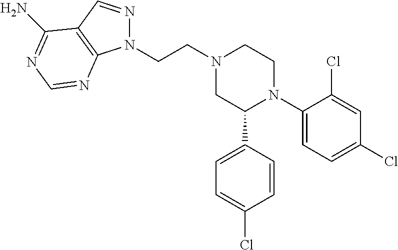 [SMILES: Nc1ncnc2c1cnn2CCN1CCN(c2ccc(Cl)cc2Cl)[C@H](c2ccc(Cl)cc2)C1] | 504.3 | 2.65 |
| 1140 | 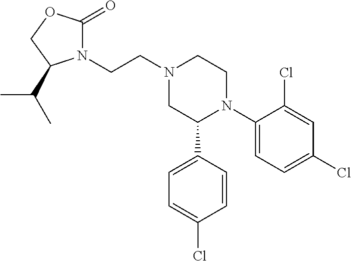 [SMILES: CC(C)[C@H]1COC(=O)N1CCN1CCN(c2ccc(Cl)cc2Cl)[C@H](c2ccc(Cl)cc2)C1] | 498.3 | 3.65 |
| 1141 | 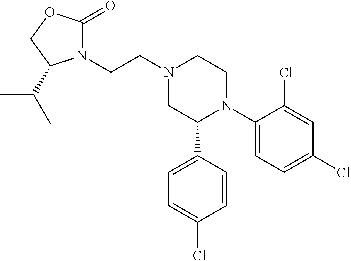 [SMILES: CC(C)[C@@H]1COC(=O)N1CCN1CCN(c2ccc(Cl)cc2Cl)[C@H](c2ccc(Cl)cc2)C1] | 498.3 | 3.61 |
| 1142 | 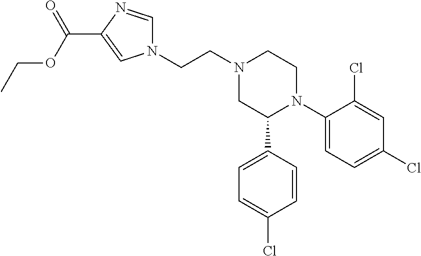 [SMILES: CCOC(=O)c1cn(CCN2CCN(c3ccc(Cl)cc3Cl)[C@H](c3ccc(Cl)cc3)C2)cn1] | 509.3 | 3.40 |
| 1143 | 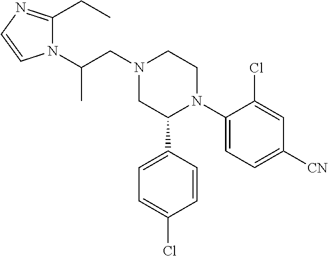 [SMILES: CCc1nccn1C(C)CN1CCN(c2ccc(C#N)cc2Cl)[C@H](c2ccc(Cl)cc2)C1] | 468.3 | 3.04 |
| 1144 | 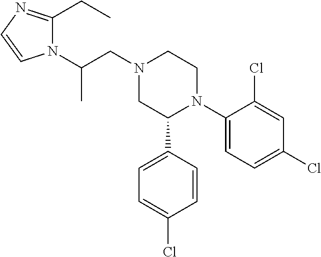 [SMILES: CCc1nccn1C(C)CN1CCN(c2ccc(Cl)cc2Cl)[C@H](c2ccc(Cl)cc2)C1] | 477.3 | 3.25 |
| 1145 | 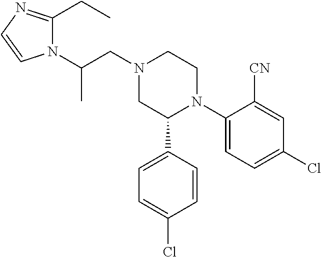 [SMILES: CCc1nccn1C(C)CN1CCN(c2ccc(Cl)cc2C#N)[C@H](c2ccc(Cl)cc2)C1] | 468.3 | 3.11 |
| 1146 | 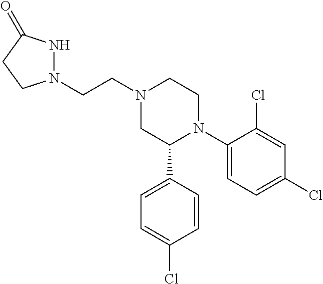 [SMILES: O=C1CCN(CCN2CCN(c3ccc(Cl)cc3Cl)[C@H](c3ccc(Cl)cc3)C2)N1] | 453.2 | 3.41 |
| 1147 | 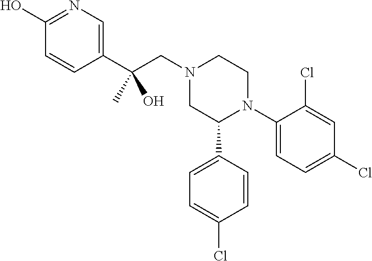 [SMILES: C[C@@](O)(CN1CCN(c2ccc(Cl)cc2Cl)[C@H](c2ccc(Cl)cc2)C1)c1ccc(O)nc1] | 492.3 | 2.89 |
| 1148 | 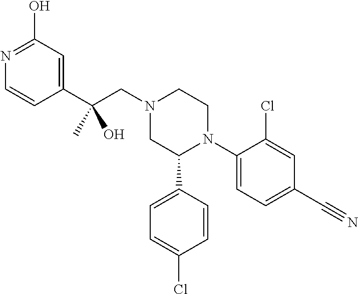 [SMILES: C[C@@](O)(CN1CCN(c2ccc(C#N)cc2Cl)[C@H](c2ccc(Cl)cc2)C1)c1ccnc(O)c1] | 483.3 | 2.80 |
| 1149 | 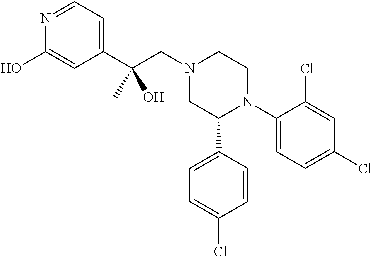 [SMILES: C[C@@](O)(CN1CCN(c2ccc(Cl)cc2Cl)[C@H](c2ccc(Cl)cc2)C1)c1ccnc(O)c1] | 492.3 | 3.03 |
| 1150 | 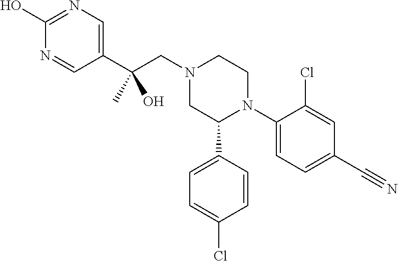 [SMILES: C[C@@](O)(CN1CCN(c2ccc(C#N)cc2Cl)[C@H](c2ccc(Cl)cc2)C1)c1cnc(O)nc1] | 484.3 | 2.79 |
| 1151 | 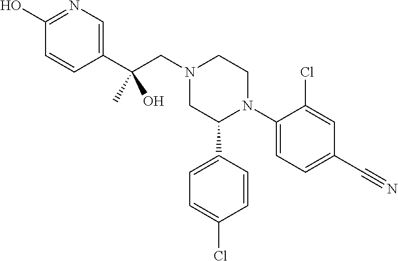 [SMILES: C[C@@](O)(CN1CCN(c2ccc(C#N)cc2Cl)[C@H](c2ccc(Cl)cc2)C1)c1ccc(O)nc1] | 483.3 | 2.76 |
| 1152 | 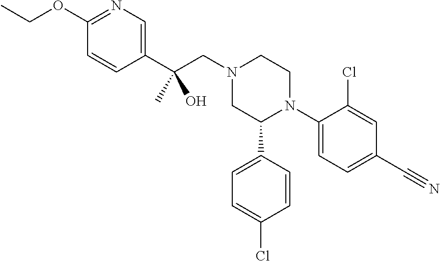 [SMILES: CCOc1ccc([C@](C)(O)CN2CCN(c3ccc(C#N)cc3Cl)[C@H](c3ccc(Cl)cc3)C2)cn1] | 511.3 | 3.29 |
| 1153 | 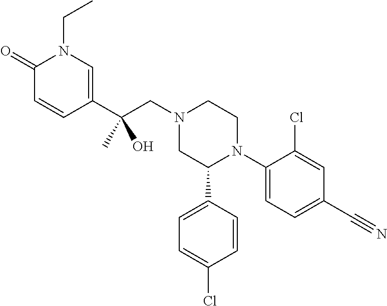 [SMILES: CCn1cc([C@](C)(O)CN2CCN(c3ccc(C#N)cc3Cl)[C@H](c3ccc(Cl)cc3)C2)ccc1=O] | 511.3 | 2.90 |
| 1154 | 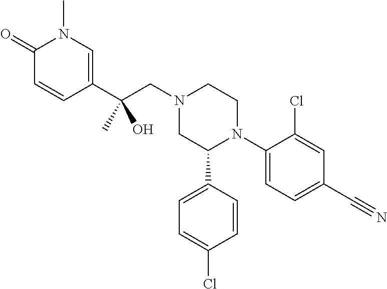 [SMILES: Cn1cc([C@](C)(O)CN2CCN(c3ccc(C#N)cc3Cl)[C@H](c3ccc(Cl)cc3)C2)ccc1=O] | 497.3 | 2.86 |
| 1155b | 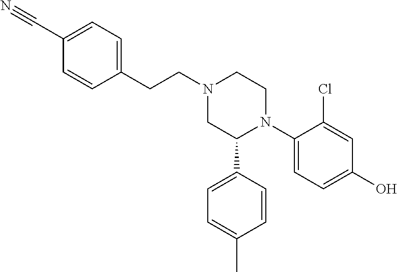 [SMILES: Cc1ccc([C@@H]2CN(CCc3ccc(C#N)cc3)CCN2c2ccc(O)cc2Cl)cc1] | 432.2 | 3.37 |
| 1156 | 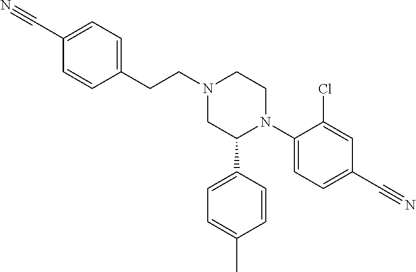 [SMILES: Cc1ccc([C@@H]2CN(CCc3ccc(C#N)cc3)CCN2c2ccc(C#N)cc2Cl)cc1] | 441.2 | 3.40 |
| 1158 | 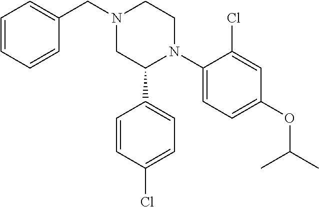 [SMILES: CC(C)Oc1ccc(N2CCN(Cc3ccccc3)C[C@H]2c2ccc(Cl)cc2)c(Cl)c1] | 455.3 | 3.64 |
| 1159 | 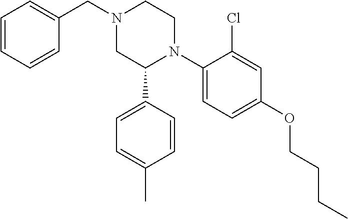 [SMILES: CCCCOc1ccc(N2CCN(Cc3ccccc3)C[C@H]2c2ccc(C)cc2)c(Cl)c1] | 449.2 | 4.30 |
| 1160 | 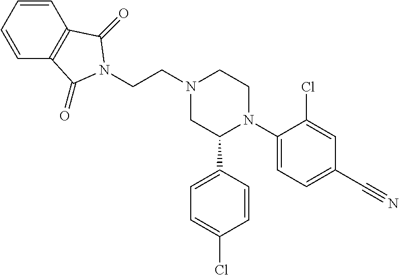 [SMILES: N#Cc1ccc(N2CCN(CCN3C(=O)c4ccccc4C3=O)C[C@H]2c2ccc(Cl)cc2)c(Cl)c1] | 505.3 | 3.68 |
| 1161 | 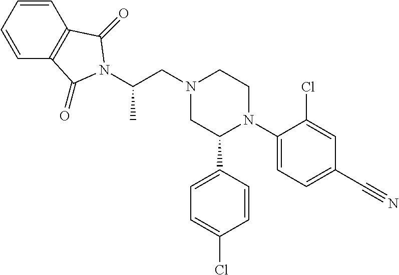 [SMILES: C[C@@H](CN1CCN(c2ccc(C#N)cc2Cl)[C@H](c2ccc(Cl)cc2)C1)N1C(=O)c2ccccc2C1=O] | 519.3 | 3.80 |
| 1162 | 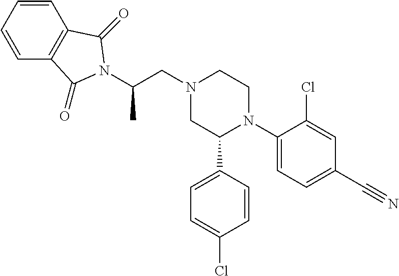 [SMILES: C[C@H](CN1CCN(c2ccc(C#N)cc2Cl)[C@H](c2ccc(Cl)cc2)C1)N1C(=O)c2ccccc2C1=O] | 519.3 | 3.90 |
| 1163 | 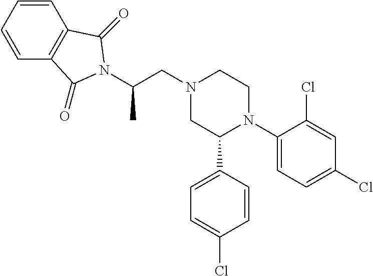 [SMILES: C[C@H](CN1CCN(c2ccc(Cl)cc2Cl)[C@H](c2ccc(Cl)cc2)C1)N1C(=O)c2ccccc2C1=O] | 528.3 | 4.12 |
| 1164 | 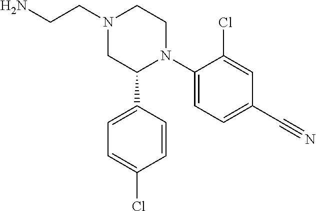 [SMILES: N#Cc1ccc(N2CCN(CCN)C[C@H]2c2ccc(Cl)cc2)c(Cl)c1] | 375.2 | 2.35 |
| 1165 | 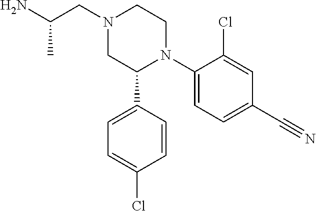 [SMILES: C[C@H](N)CN1CCN(c2ccc(C#N)cc2Cl)[C@H](c2ccc(Cl)cc2)C1] | 389.2 | 2.84 |
| 1167a | 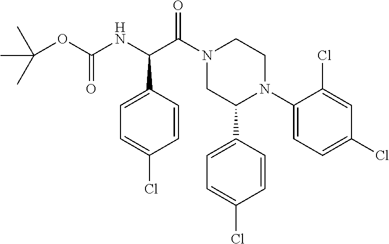 [SMILES: CC(C)(C)OC(=O)N[C@@H](C(=O)N1CCN(c2ccc(Cl)cc2Cl)[C@H](c2ccc(Cl)cc2)C1)c1ccc(Cl)cc1] | 610.3 | 6.40 |
| 1167b | 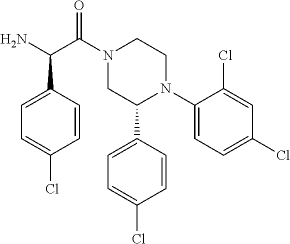 [SMILES: N[C@@H](C(=O)N1CCN(c2ccc(Cl)cc2Cl)[C@H](c2ccc(Cl)cc2)C1)c1ccc(Cl)cc1] | 510.3 | 4.33 |
| 1168 | 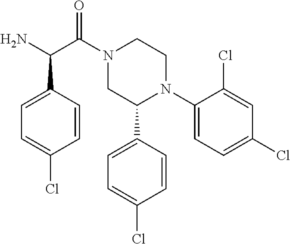 [SMILES: N[C@@H](C(=O)N1CCN(c2ccc(Cl)cc2Cl)[C@H](c2ccc(Cl)cc2)C1)c1ccc(Cl)cc1] | 496.3 | 4.52 |
| 1169 | 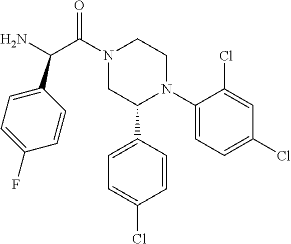 [SMILES: N[C@@H](C(=O)N1CCN(c2ccc(Cl)cc2Cl)[C@H](c2ccc(Cl)cc2)C1)c1ccc(F)cc1] | 492.3 | 3.69 |
| 1170 | 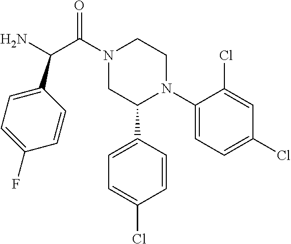 [SMILES: N[C@@H](C(=O)N1CCN(c2ccc(Cl)cc2Cl)[C@H](c2ccc(Cl)cc2)C1)c1ccc(F)cc1] | 480.3 | 4.26 |
| 1171 | 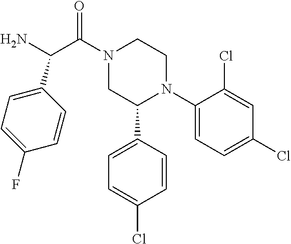 [SMILES: N[C@H](C(=O)N1CCN(c2ccc(Cl)cc2Cl)[C@H](c2ccc(Cl)cc2)C1)c1ccc(F)cc1] | 492.3 | 3.70 |
| 1172 | 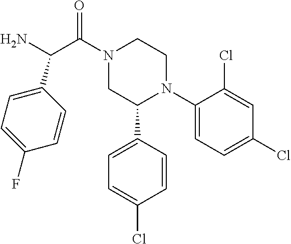 [SMILES: N[C@H](C(=O)N1CCN(c2ccc(Cl)cc2Cl)[C@H](c2ccc(Cl)cc2)C1)c1ccc(F)cc1] | 480.3 | 4.34 |
| 1173 | 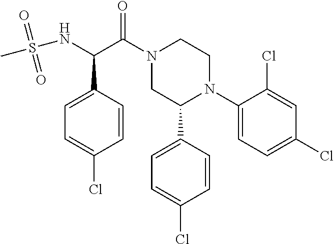 [SMILES: CS(=O)(=O)N[C@@H](C(=O)N1CCN(c2ccc(Cl)cc2Cl)[C@H](c2ccc(Cl)cc2)C1)c1ccc(Cl)cc1] | 574.3 | 4.02 |
| 1174 | 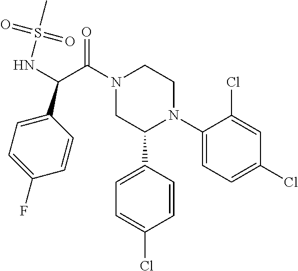 [SMILES: CS(=O)(=O)N[C@@H](C(=O)N1CCN(c2ccc(Cl)cc2Cl)[C@H](c2ccc(Cl)cc2)C1)c1ccc(F)cc1] | 556.3 | 4.21 |
| 1175 | 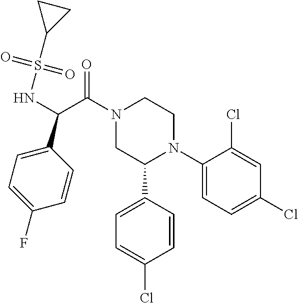 [SMILES: O=C([C@H](NS(=O)(=O)C1CC1)c1ccc(F)cc1)N1CCN(c2ccc(Cl)cc2Cl)[C@H](c2ccc(Cl)cc2)C1] | 582.3 | 4.37 |
| 1176 | 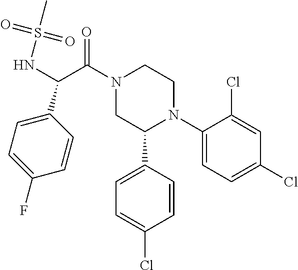 [SMILES: CS(=O)(=O)N[C@H](C(=O)N1CCN(c2ccc(Cl)cc2Cl)[C@H](c2ccc(Cl)cc2)C1)c1ccc(F)cc1] | 556.3 | 4.21 |
| 1177 | 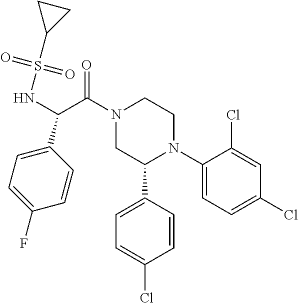 [SMILES: O=C([C@@H](NS(=O)(=O)C1CC1)c1ccc(F)cc1)N1CCN(c2ccc(Cl)cc2Cl)[C@H](c2ccc(Cl)cc2)C1] | 584.3 | 4.42 |
| 1178 | 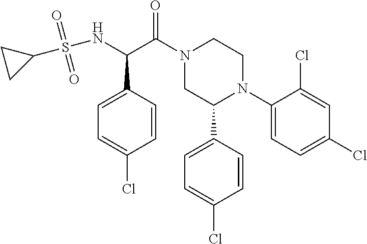 [SMILES: O=C([C@H](NS(=O)(=O)C1CC1)c1ccc(Cl)cc1)N1CCN(c2ccc(Cl)cc2Cl)[C@H](c2ccc(Cl)cc2)C1] | 600.3 | 4.13 |
| 1179 | 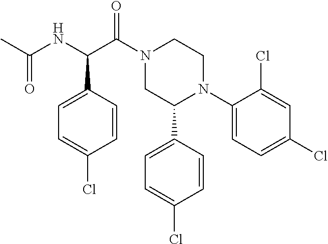 [SMILES: CC(=O)N[C@@H](C(=O)N1CCN(c2ccc(Cl)cc2Cl)[C@H](c2ccc(Cl)cc2)C1)c1ccc(Cl)cc1] | 538.3 | 3.89 |
| 1180 | 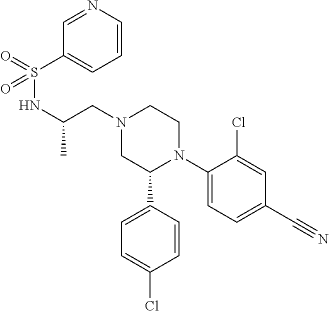 [SMILES: C[C@@H](CN1CCN(c2ccc(C#N)cc2Cl)[C@H](c2ccc(Cl)cc2)C1)NS(=O)(=O)c1cccnc1] | 530.3 | 2.91 |
| 1181 | 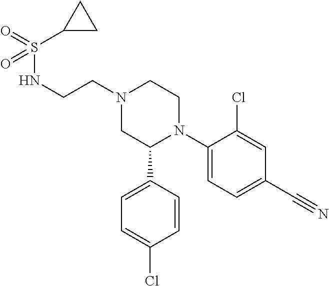 [SMILES: N#Cc1ccc(N2CCN(CCNS(=O)(=O)C3CC3)C[C@H]2c2ccc(Cl)cc2)c(Cl)c1] | 479.3 | 2.89 |
| 1182 | 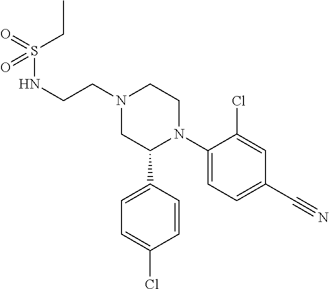 [SMILES: CCS(=O)(=O)NCCN1CCN(c2ccc(C#N)cc2Cl)[C@H](c2ccc(Cl)cc2)C1] | 467.3 | 2.84 |
| 1183 | 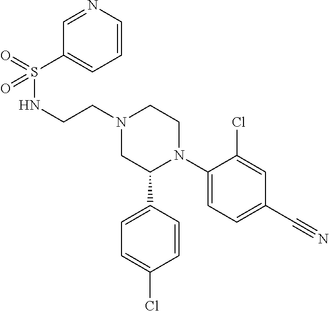 [SMILES: N#Cc1ccc(N2CCN(CCNS(=O)(=O)c3cccnc3)C[C@H]2c2ccc(Cl)cc2)c(Cl)c1] | 576.3 | 2.90 |
| 1184 | 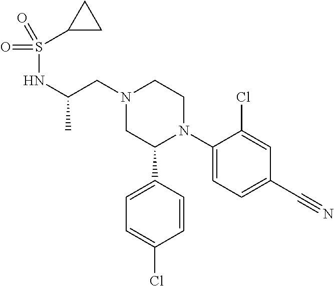 [SMILES: C[C@@H](CN1CCN(c2ccc(C#N)cc2Cl)[C@H](c2ccc(Cl)cc2)C1)NS(=O)(=O)C1CC1] | 493.3 | 2.95 |
| 1185 | 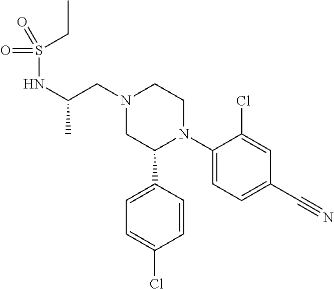 [SMILES: CCS(=O)(=O)N[C@@H](C)CN1CCN(c2ccc(C#N)cc2Cl)[C@H](c2ccc(Cl)cc2)C1] | 481.3 | 2.90 |
| 1186 | 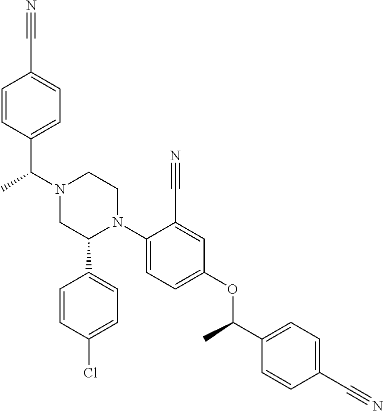 [SMILES: C[C@H](c1ccc(C#N)cc1)N1CCN(c2ccc(O[C@H](C)c3ccc(C#N)cc3)cc2C#N)[C@H](c2ccc(Cl)cc2)C1] | 572.3 | 4.00 |

We claim:

1. A compound, or a pharmaceutically acceptable salt thereof, said compound being selected from the group consisting of:

| Example | Structure |
|---|---|
| 803 | 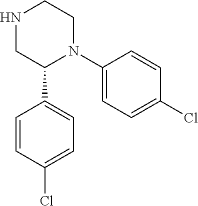 [SMILES: Clc1ccc([C@@H]2CNCCN2c2ccc(Cl)cc2)cc1] |
| 805 | 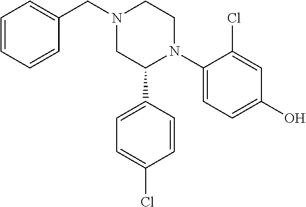 [SMILES: Oc1ccc(N2CCN(Cc3ccccc3)C[C@H]2c2ccc(Cl)cc2)c(Cl)c1] |
| 806 | 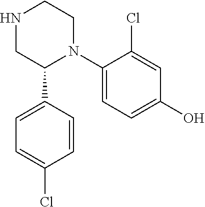 [SMILES: Oc1ccc(N2CCNC[C@H]2c2ccc(Cl)cc2)c(Cl)c1] |
| 807 | 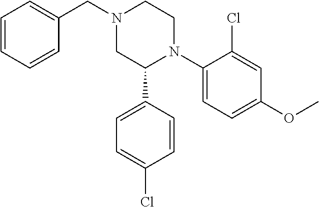 [SMILES: COc1ccc(N2CCN(Cc3ccccc3)C[C@H]2c2ccc(Cl)cc2)c(Cl)c1] |
| 808 | 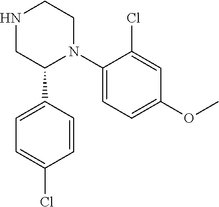 [SMILES: COc1ccc(N2CCNC[C@H]2c2ccc(Cl)cc2)c(Cl)c1] |
| 809 | 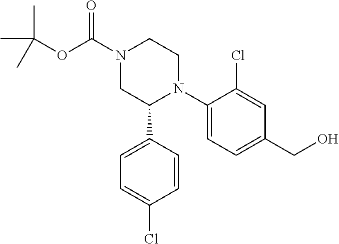 [SMILES: CC(C)(C)OC(=O)N1CCN(c2ccc(CO)cc2Cl)[C@H](c2ccc(Cl)cc2)C1] |
| 811 | 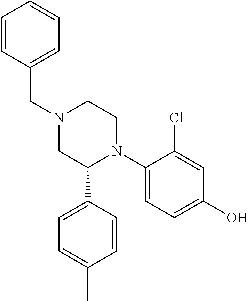 [SMILES: Cc1ccc([C@@H]2CN(Cc3ccccc3)CCN2c2ccc(O)cc2Cl)cc1] |
| 812b | 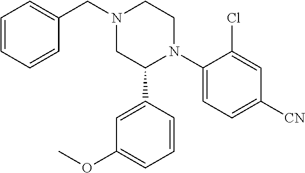 [SMILES: COc1cccc([C@@H]2CN(Cc3ccccc3)CCN2c2ccc(C#N)cc2Cl)c1] |
| 812 | 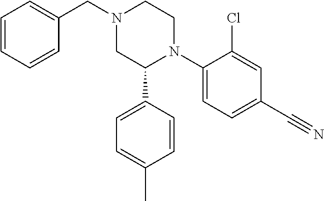 [SMILES: Cc1ccc([C@@H]2CN(Cc3ccccc3)CCN2c2ccc(C#N)cc2Cl)cc1] |
| 813b | 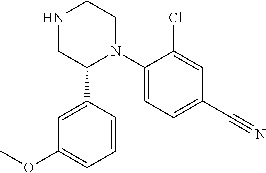 [SMILES: COc1cccc([C@@H]2CNCCN2c2ccc(C#N)cc2Cl)c1] |
| 813a | 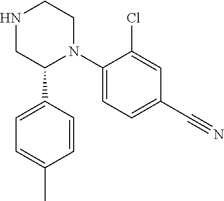 [SMILES: Cc1ccc([C@@H]2CNCCN2c2ccc(C#N)cc2Cl)cc1] |
| 814 | 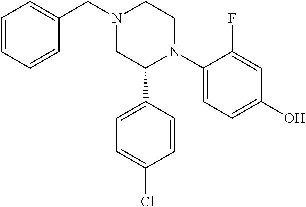 [SMILES: Oc1ccc(N2CCN(Cc3ccccc3)C[C@H]2c2ccc(Cl)cc2)c(F)c1] |
| 815 | 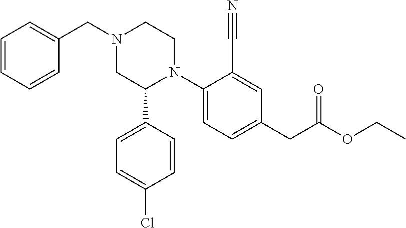 [SMILES: CCOC(=O)Cc1ccc(N2CCN(Cc3ccccc3)C[C@H]2c2ccc(Cl)cc2)c(C#N)c1] |
| 816 | 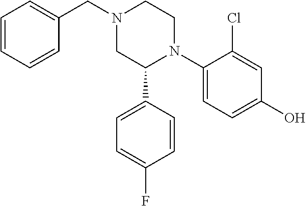 [SMILES: Oc1ccc(N2CCN(Cc3ccccc3)C[C@H]2c2ccc(F)cc2)c(Cl)c1] |
| 817 | 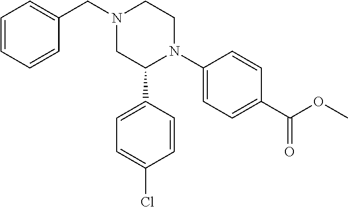 [SMILES: COC(=O)c1ccc(N2CCN(Cc3ccccc3)C[C@H]2c2ccc(Cl)cc2)cc1] |
| 818 | 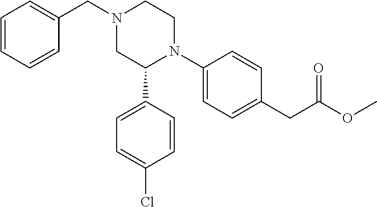 [SMILES: COC(=O)Cc1ccc(N2CCN(Cc3ccccc3)C[C@H]2c2ccc(Cl)cc2)cc1] |
| 819 | 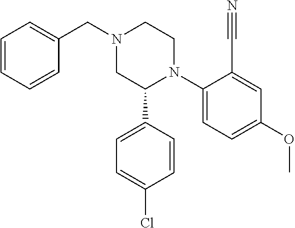 [SMILES: COc1ccc(N2CCN(Cc3ccccc3)C[C@H]2c2ccc(Cl)cc2)c(C#N)c1] |
| 820 | 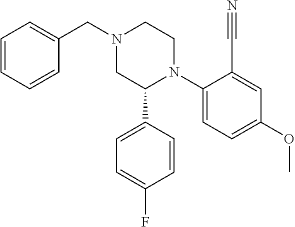 [SMILES: COc1ccc(N2CCN(Cc3ccccc3)C[C@H]2c2ccc(F)cc2)c(C#N)c1] |
| 820b | 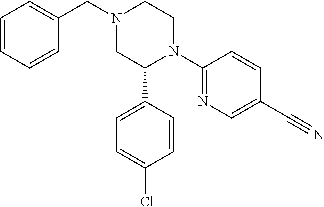 [SMILES: N#Cc1ccc(N2CCN(Cc3ccccc3)C[C@H]2c2ccc(Cl)cc2)nc1] |
| 820d | 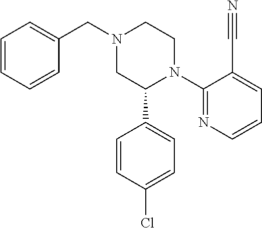 [SMILES: N#Cc1cccnc1N1CCN(Cc2ccccc2)C[C@H]1c1ccc(Cl)cc1] |
| 820e | 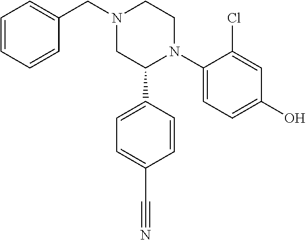 [SMILES: N#Cc1ccc([C@@H]2CN(Cc3ccccc3)CCN2c2ccc(O)cc2Cl)cc1] |
| 821 | 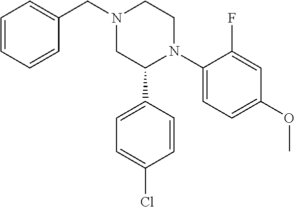 [SMILES: COc1ccc(N2CCN(Cc3ccccc3)C[C@H]2c2ccc(Cl)cc2)c(F)c1] |
| 822 | 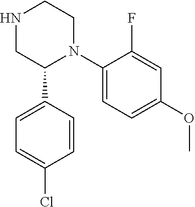 [SMILES: COc1ccc(N2CCNC[C@H]2c2ccc(Cl)cc2)c(F)c1] |
| 823 | 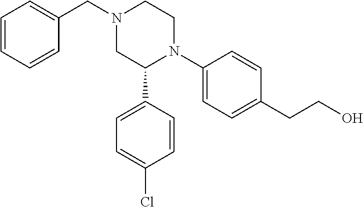 [SMILES: OCCc1ccc(N2CCN(Cc3ccccc3)C[C@H]2c2ccc(Cl)cc2)cc1] |
| 824 | 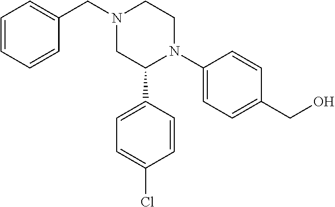 [SMILES: OCc1ccc(N2CCN(Cc3ccccc3)C[C@H]2c2ccc(Cl)cc2)cc1] |
| 826 | 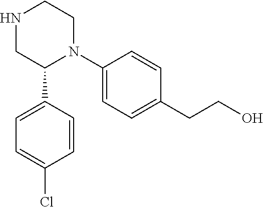 [SMILES: OCCc1ccc(N2CCNC[C@H]2c2ccc(Cl)cc2)cc1] |
| 826b | 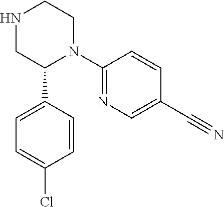 [SMILES: N#Cc1ccc(N2CCNC[C@H]2c2ccc(Cl)cc2)nc1] |
| 826a | 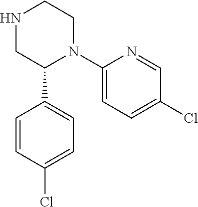 [SMILES: Clc1ccc([C@@H]2CNCCN2c2ccc(Cl)cn2)cc1] |
| 826d | 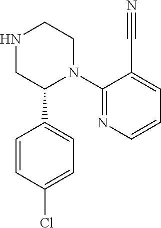 [SMILES: N#Cc1cccnc1N1CCNC[C@H]1c1ccc(Cl)cc1] |
| 826e | 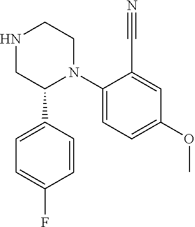 [SMILES: COc1ccc(N2CCNC[C@H]2c2ccc(F)cc2)c(C#N)c1] |
| 827 | 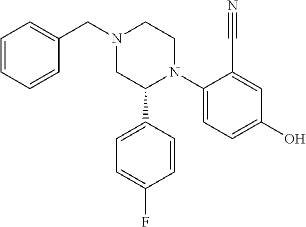 [SMILES: N#Cc1cc(O)ccc1N1CCN(Cc2ccccc2)C[C@H]1c1ccc(F)cc1] |
| 828 | 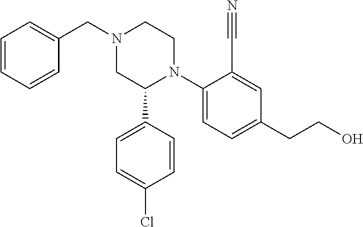 [SMILES: N#Cc1cc(CCO)ccc1N1CCN(Cc2ccccc2)C[C@H]1c1ccc(Cl)cc1] |
| 829 | 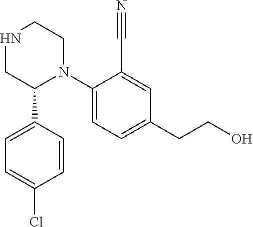 [SMILES: N#Cc1cc(CCO)ccc1N1CCNC[C@H]1c1ccc(Cl)cc1] |
| 830 | 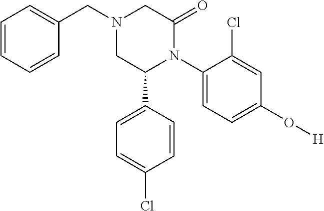 [SMILES: O=C1CN(Cc2ccccc2)C[C@@H](c2ccc(Cl)cc2)N1c1ccc(O)cc1Cl] |
| 831 | 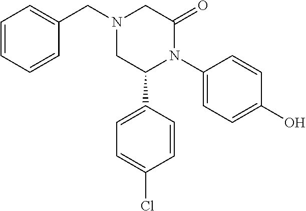 [SMILES: O=C1CN(Cc2ccccc2)C[C@@H](c2ccc(Cl)cc2)N1c1ccc(O)cc1] |
| 832 | 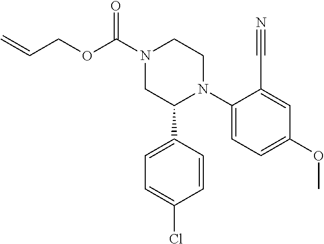 [SMILES: C=CCOC(=O)N1CCN(c2ccc(OC)cc2C#N)[C@H](c2ccc(Cl)cc2)C1] |
| 833 | 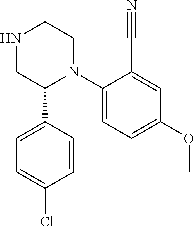 [SMILES: COc1ccc(N2CCNC[C@H]2c2ccc(Cl)cc2)c(C#N)c1] |
| 834 | 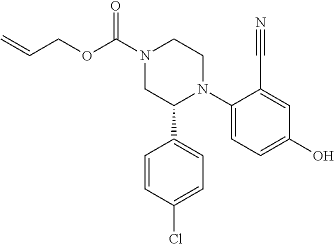 [SMILES: C=CCOC(=O)N1CCN(c2ccc(O)cc2C#N)[C@H](c2ccc(Cl)cc2)C1] |
| 835 | 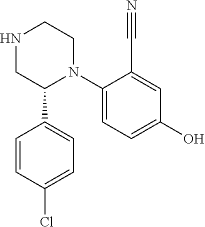 [SMILES: N#Cc1cc(O)ccc1N1CCNC[C@H]1c1ccc(Cl)cc1] |
| 836 | 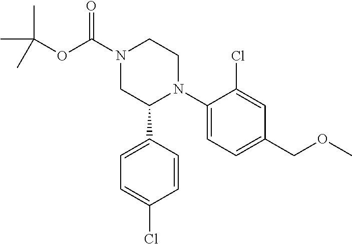 [SMILES: COCc1ccc(N2CCN(C(=O)OC(C)(C)C)C[C@H]2c2ccc(Cl)cc2)c(Cl)c1] |
| 837 | 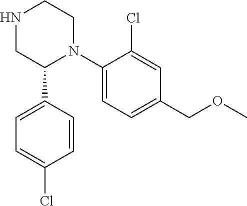 [SMILES: COCc1ccc(N2CCNC[C@H]2c2ccc(Cl)cc2)c(Cl)c1] |
| 839 | 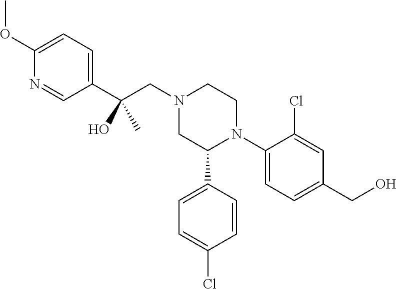 [SMILES: COc1ccc([C@](C)(O)CN2CCN(c3ccc(CO)cc3Cl)[C@H](c3ccc(Cl)cc3)C2)cn1] |
| 840 | 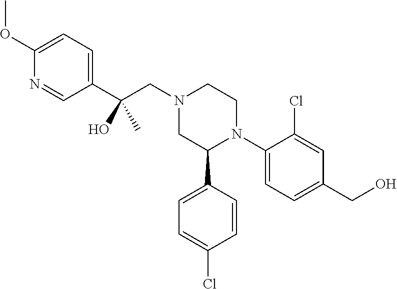 [SMILES: COc1ccc([C@](C)(O)CN2CCN(c3ccc(CO)cc3Cl)[C@@H](c3ccc(Cl)cc3)C2)cn1] |
| 841 | 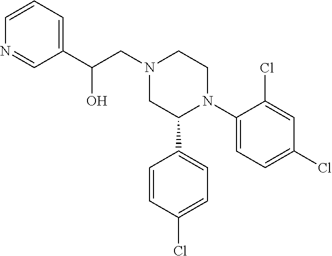 [SMILES: OC(CN1CCN(c2ccc(Cl)cc2Cl)[C@H](c2ccc(Cl)cc2)C1)c1cccnc1] |
| 842 | 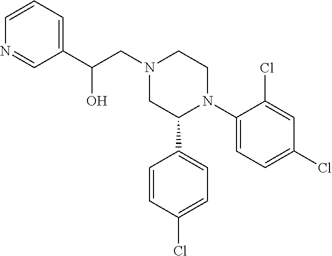 [SMILES: OC(CN1CCN(c2ccc(Cl)cc2Cl)[C@H](c2ccc(Cl)cc2)C1)c1cccnc1] |
| 842c | 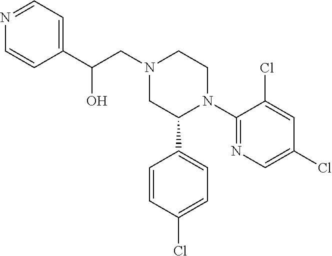 [SMILES: OC(CN1CCN(c2ncc(Cl)cc2Cl)[C@H](c2ccc(Cl)cc2)C1)c1ccncc1] |
| 844 | 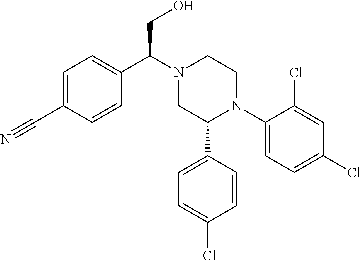 [SMILES: N#Cc1ccc([C@@H](CO)N2CCN(c3ccc(Cl)cc3Cl)[C@H](c3ccc(Cl)cc3)C2)cc1] |
| 845 | 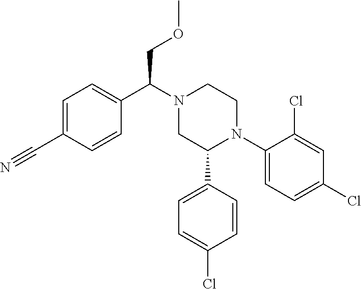 [SMILES: COC[C@H](c1ccc(C#N)cc1)N1CCN(c2ccc(Cl)cc2Cl)[C@H](c2ccc(Cl)cc2)C1] |
| 846 | 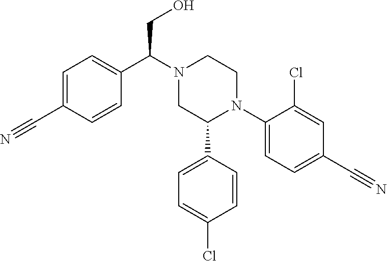 [SMILES: N#Cc1ccc([C@@H](CO)N2CCN(c3ccc(C#N)cc3Cl)[C@H](c3ccc(Cl)cc3)C2)cc1] |
| 847a | 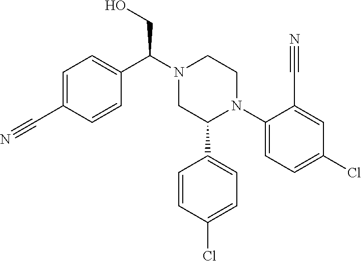 [SMILES: N#Cc1ccc([C@@H](CO)N2CCN(c3ccc(Cl)cc3C#N)[C@H](c3ccc(Cl)cc3)C2)cc1] |
| 847 | 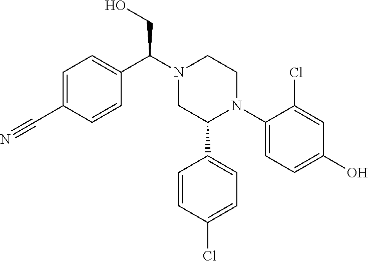 [SMILES: N#Cc1ccc([C@@H](CO)N2CCN(c3ccc(O)cc3Cl)[C@H](c3ccc(Cl)cc3)C2)cc1] |
| 848 | 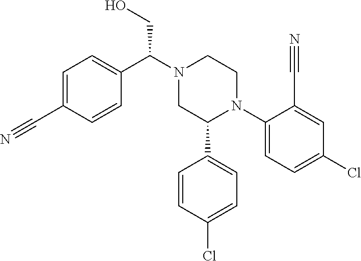 [SMILES: N#Cc1ccc([C@H](CO)N2CCN(c3ccc(Cl)cc3C#N)[C@H](c3ccc(Cl)cc3)C2)cc1] |
| 850 | 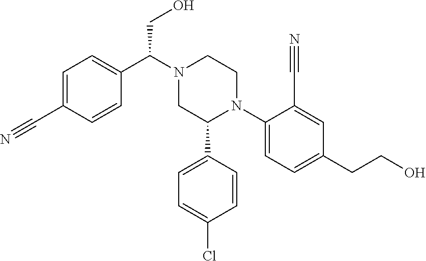 [SMILES: N#Cc1ccc([C@H](CO)N2CCN(c3ccc(CCO)cc3C#N)[C@H](c3ccc(Cl)cc3)C2)cc1] |
| 851 | 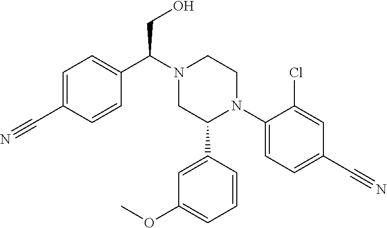 [SMILES: COc1cccc([C@@H]2CN([C@H](CO)c3ccc(C#N)cc3)CCN2c2ccc(C#N)cc2Cl)c1] |
| 852 | 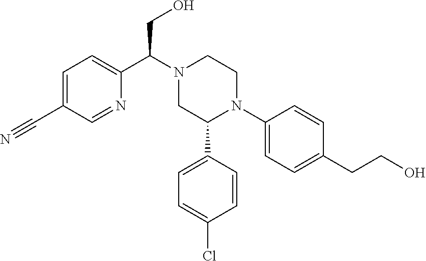 [SMILES: N#Cc1ccc([C@@H](CO)N2CCN(c3ccc(CCO)cc3)[C@H](c3ccc(Cl)cc3)C2)nc1] |
| 853 | 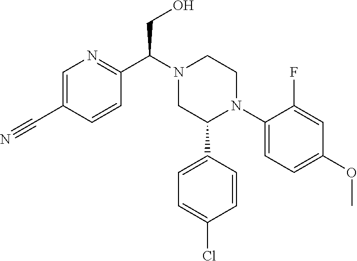 [SMILES: COc1ccc(N2CCN([C@H](CO)c3ccc(C#N)cn3)C[C@H]2c2ccc(Cl)cc2)c(F)c1] |
| 854 | 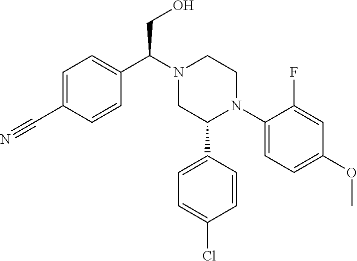 [SMILES: COc1ccc(N2CCN([C@H](CO)c3ccc(C#N)cc3)C[C@H]2c2ccc(Cl)cc2)c(F)c1] |
| 855 | 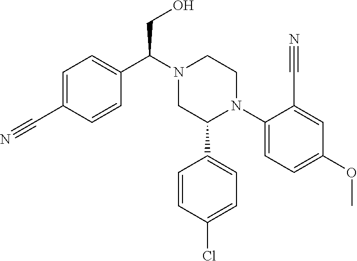 [SMILES: COc1ccc(N2CCN([C@H](CO)c3ccc(C#N)cc3)C[C@H]2c2ccc(Cl)cc2)c(C#N)c1] |
| 856 | 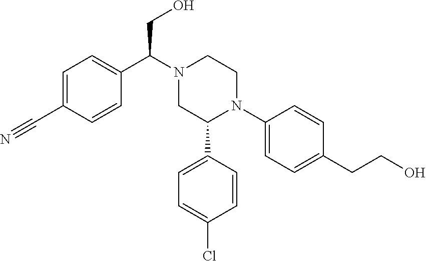 [SMILES: N#Cc1ccc([C@@H](CO)N2CCN(c3ccc(CCO)cc3)[C@H](c3ccc(Cl)cc3)C2)cc1] |
| 857a | 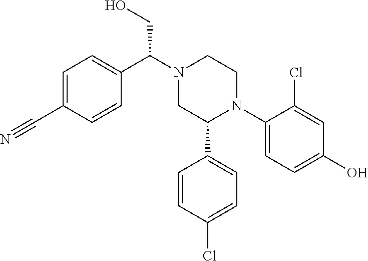 [SMILES: N#Cc1ccc([C@H](CO)N2CCN(c3ccc(O)cc3Cl)[C@H](c3ccc(Cl)cc3)C2)cc1] |
| 857 | 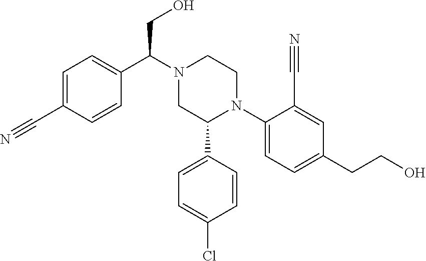 [SMILES: N#Cc1ccc([C@@H](CO)N2CCN(c3ccc(CCO)cc3C#N)[C@H](c3ccc(Cl)cc3)C2)cc1] |
| 858 | 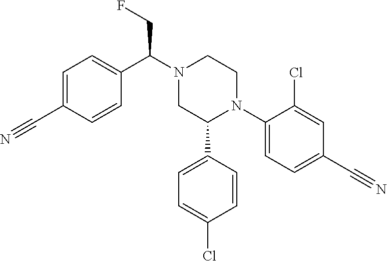 [SMILES: N#Cc1ccc([C@@H](CF)N2CCN(c3ccc(C#N)cc3Cl)[C@H](c3ccc(Cl)cc3)C2)cc1] |
| 859 | 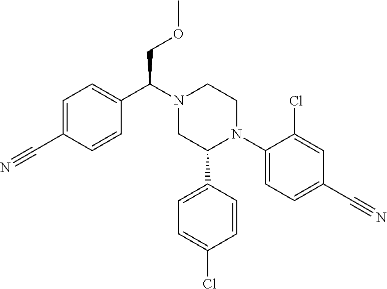 [SMILES: COC[C@H](c1ccc(C#N)cc1)N1CCN(c2ccc(C#N)cc2Cl)[C@H](c2ccc(Cl)cc2)C1] |
| 861 | 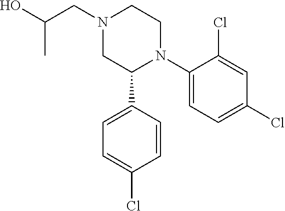 [SMILES: CC(O)CN1CCN(c2ccc(Cl)cc2Cl)[C@H](c2ccc(Cl)cc2)C1] |
| 862 | 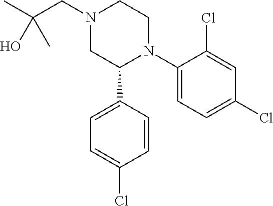 [SMILES: CC(C)(O)CN1CCN(c2ccc(Cl)cc2Cl)[C@H](c2ccc(Cl)cc2)C1] |
| 863 | 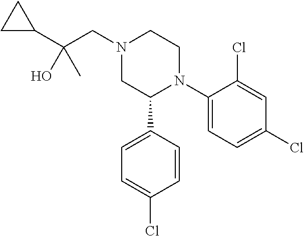 [SMILES: CC(O)(CN1CCN(c2ccc(Cl)cc2Cl)[C@H](c2ccc(Cl)cc2)C1)C1CC1] |
| 864 | 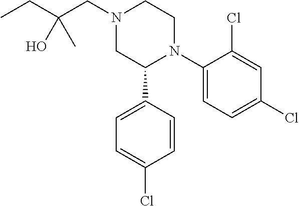 [SMILES: CCC(C)(O)CN1CCN(c2ccc(Cl)cc2Cl)[C@H](c2ccc(Cl)cc2)C1] |
| 865 | 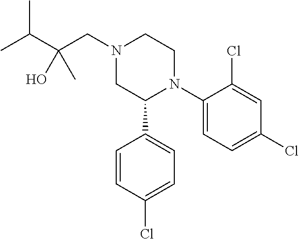 [SMILES: CC(C)C(C)(O)CN1CCN(c2ccc(Cl)cc2Cl)[C@H](c2ccc(Cl)cc2)C1] |
| 866 | 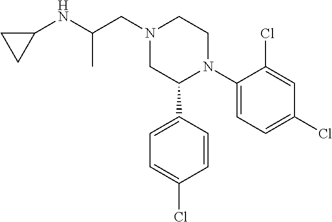 [SMILES: CC(CN1CCN(c2ccc(Cl)cc2Cl)[C@H](c2ccc(Cl)cc2)C1)NC1CC1] |
| 867 | 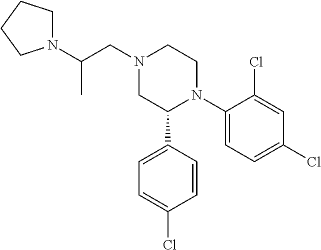 [SMILES: CC(CN1CCN(c2ccc(Cl)cc2Cl)[C@H](c2ccc(Cl)cc2)C1)N1CCCC1] |
| 868c | 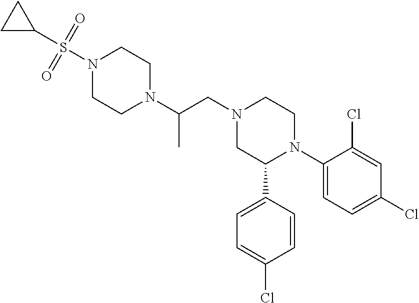 [SMILES: CC(CN1CCN(c2ccc(Cl)cc2Cl)[C@H](c2ccc(Cl)cc2)C1)N1CCN(S(=O)(=O)C2CC2)CC1] |
| 868 | 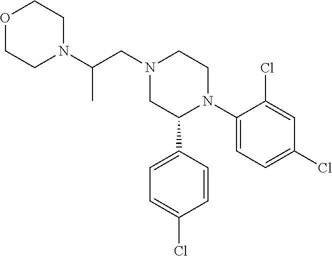 [SMILES: CC(CN1CCN(c2ccc(Cl)cc2Cl)[C@H](c2ccc(Cl)cc2)C1)N1CCOCC1] |
| 870 | 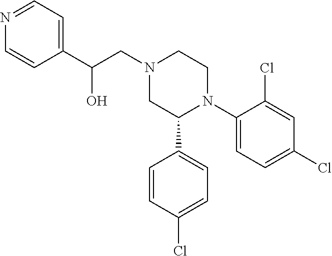 [SMILES: OC(CN1CCN(c2ccc(Cl)cc2Cl)[C@H](c2ccc(Cl)cc2)C1)c1ccncc1] |
| 872 | 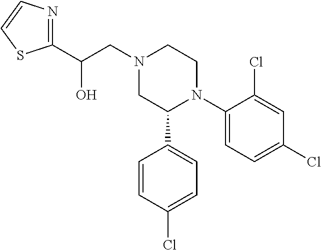 [SMILES: OC(CN1CCN(c2ccc(Cl)cc2Cl)[C@H](c2ccc(Cl)cc2)C1)c1nccs1] |
| 873 | 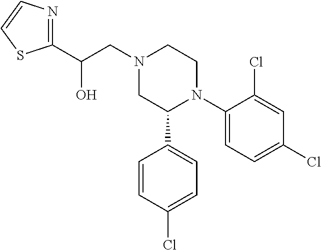 [SMILES: OC(CN1CCN(c2ccc(Cl)cc2Cl)[C@H](c2ccc(Cl)cc2)C1)c1nccs1] |
| 874 | 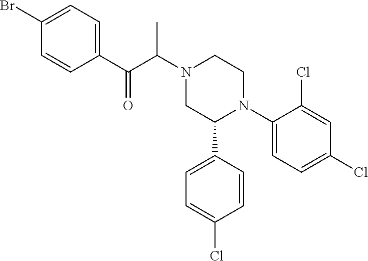 [SMILES: CC(C(=O)c1ccc(Br)cc1)N1CCN(c2ccc(Cl)cc2Cl)[C@H](c2ccc(Cl)cc2)C1] |
| 875 | 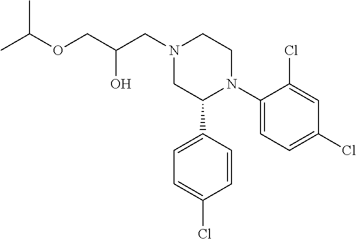 [SMILES: CC(C)OCC(O)CN1CCN(c2ccc(Cl)cc2Cl)[C@H](c2ccc(Cl)cc2)C1] |
| 876 | 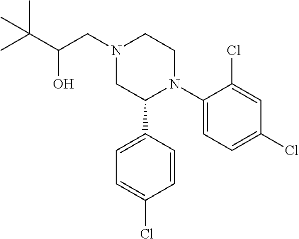 [SMILES: CC(C)(C)C(O)CN1CCN(c2ccc(Cl)cc2Cl)[C@H](c2ccc(Cl)cc2)C1] |
| 877a | 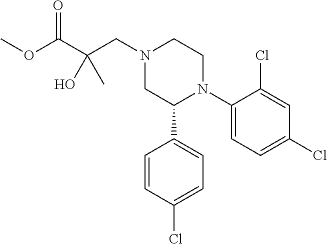 [SMILES: COC(=O)C(C)(O)CN1CCN(c2ccc(Cl)cc2Cl)[C@H](c2ccc(Cl)cc2)C1] |
| 877b | 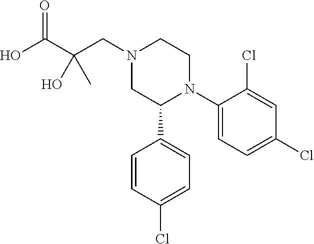 [SMILES: CC(O)(CN1CCN(c2ccc(Cl)cc2Cl)[C@H](c2ccc(Cl)cc2)C1)C(=O)O] |
| 878 | 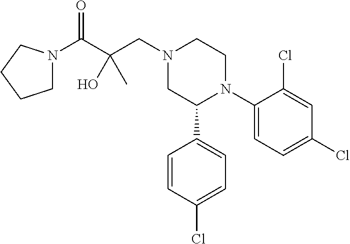 [SMILES: CC(O)(CN1CCN(c2ccc(Cl)cc2Cl)[C@H](c2ccc(Cl)cc2)C1)C(=O)N1CCCC1] |
| 879 | 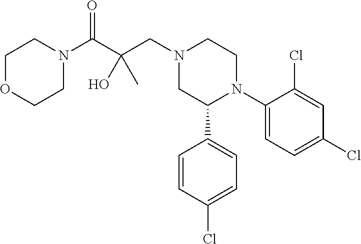 [SMILES: CC(O)(CN1CCN(c2ccc(Cl)cc2Cl)[C@H](c2ccc(Cl)cc2)C1)C(=O)N1CCOCC1] |
| 880 | 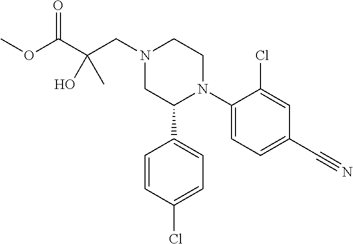 [SMILES: COC(=O)C(C)(O)CN1CCN(c2ccc(C#N)cc2Cl)[C@H](c2ccc(Cl)cc2)C1] |
| 881 | 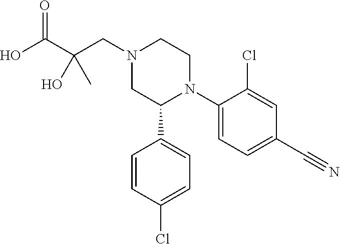 [SMILES: CC(O)(CN1CCN(c2ccc(C#N)cc2Cl)[C@H](c2ccc(Cl)cc2)C1)C(=O)O] |
| 882 | 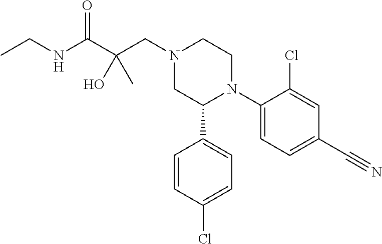 [SMILES: CCNC(=O)C(C)(O)CN1CCN(c2ccc(C#N)cc2Cl)[C@H](c2ccc(Cl)cc2)C1] |
| 883 | 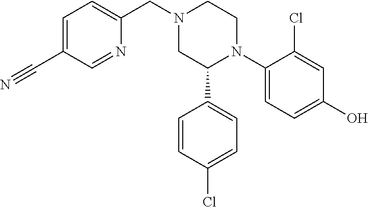 [SMILES: N#Cc1ccc(CN2CCN(c3ccc(O)cc3Cl)[C@H](c3ccc(Cl)cc3)C2)nc1] |
| 884 | 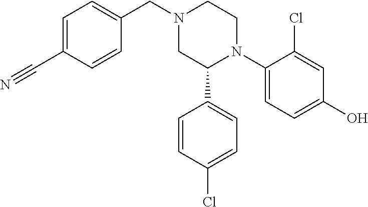 [SMILES: N#Cc1ccc(CN2CCN(c3ccc(O)cc3Cl)[C@H](c3ccc(Cl)cc3)C2)cc1] |
| 885 | 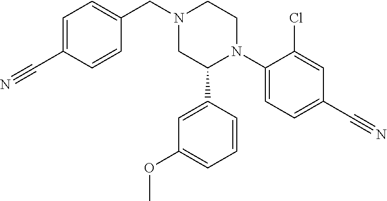 [SMILES: COc1cccc([C@@H]2CN(Cc3ccc(C#N)cc3)CCN2c2ccc(C#N)cc2Cl)c1] |
| 886 | 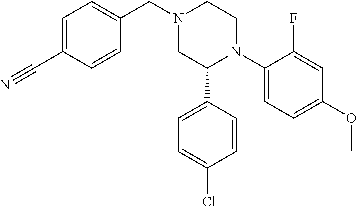 [SMILES: COc1ccc(N2CCN(Cc3ccc(C#N)cc3)C[C@H]2c2ccc(Cl)cc2)c(F)c1] |
| 887a | 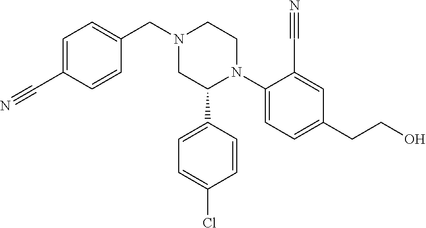 [SMILES: N#Cc1ccc(CN2CCN(c3ccc(CCO)cc3C#N)[C@H](c3ccc(Cl)cc3)C2)cc1] |
| 887b | 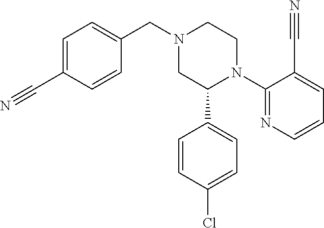 [SMILES: N#Cc1ccc(CN2CCN(c3ncccc3C#N)[C@H](c3ccc(Cl)cc3)C2)cc1] |
| 888 | 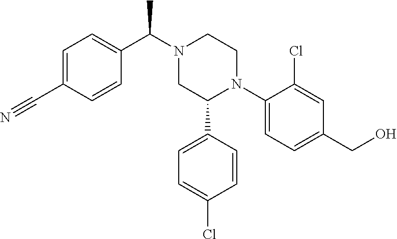 [SMILES: C[C@H](c1ccc(C#N)cc1)N1CCN(c2ccc(CO)cc2Cl)[C@H](c2ccc(Cl)cc2)C1] |
| 889 | 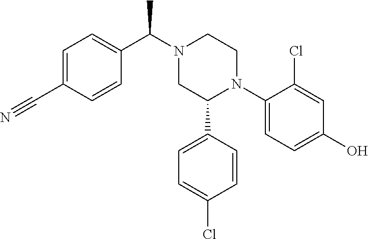 [SMILES: C[C@H](c1ccc(C#N)cc1)N1CCN(c2ccc(O)cc2Cl)[C@H](c2ccc(Cl)cc2)C1] |
| 890 | 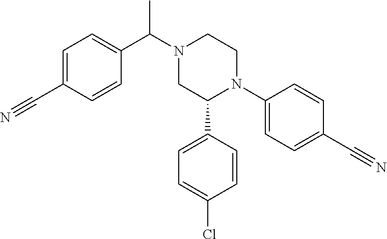 [SMILES: CC(c1ccc(C#N)cc1)N1CCN(c2ccc(C#N)cc2)[C@H](c2ccc(Cl)cc2)C1] |
| 892 | 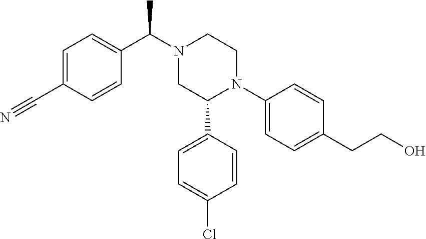 [SMILES: C[C@H](c1ccc(C#N)cc1)N1CCN(c2ccc(CCO)cc2)[C@H](c2ccc(Cl)cc2)C1] |
| 893 | 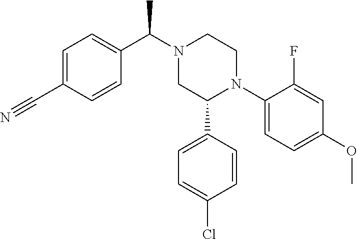 [SMILES: COc1ccc(N2CCN([C@H](C)c3ccc(C#N)cc3)C[C@H]2c2ccc(Cl)cc2)c(F)c1] |
| 895 | 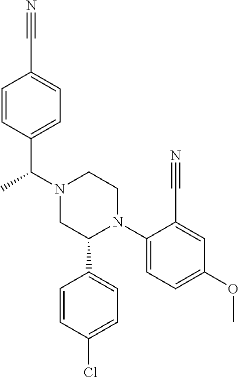 [SMILES: COc1ccc(N2CCN([C@H](C)c3ccc(C#N)cc3)C[C@H]2c2ccc(Cl)cc2)c(C#N)c1] |
| 896 | 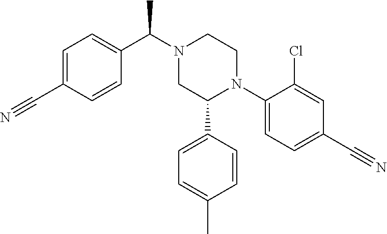 [SMILES: Cc1ccc([C@@H]2CN([C@H](C)c3ccc(C#N)cc3)CCN2c2ccc(C#N)cc2Cl)cc1] |
| 897 | 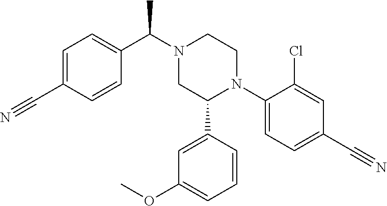 [SMILES: COc1cccc([C@@H]2CN([C@H](C)c3ccc(C#N)cc3)CCN2c2ccc(C#N)cc2Cl)c1] |
| 899 | 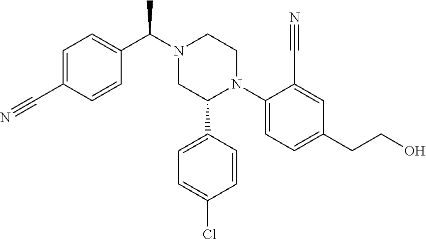 [SMILES: C[C@H](c1ccc(C#N)cc1)N1CCN(c2ccc(CCO)cc2C#N)[C@H](c2ccc(Cl)cc2)C1] |
| 899b | 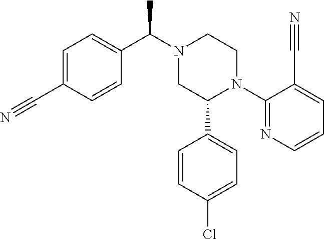 [SMILES: C[C@H](c1ccc(C#N)cc1)N1CCN(c2ncccc2C#N)[C@H](c2ccc(Cl)cc2)C1] |
| 900 | 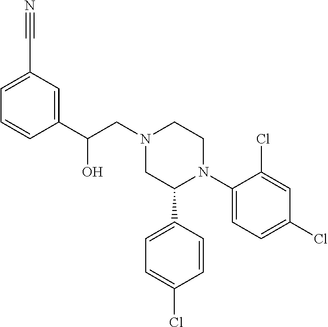 [SMILES: N#Cc1cccc(C(O)CN2CCN(c3ccc(Cl)cc3Cl)[C@H](c3ccc(Cl)cc3)C2)c1] |
| 901 | 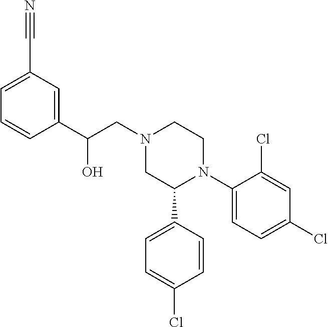 [SMILES: N#Cc1cccc(C(O)CN2CCN(c3ccc(Cl)cc3Cl)[C@H](c3ccc(Cl)cc3)C2)c1] |
| 902 | 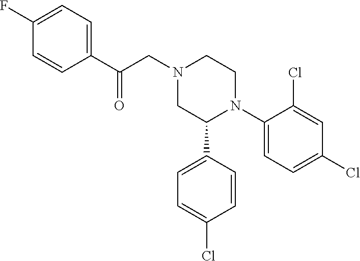 [SMILES: O=C(CN1CCN(c2ccc(Cl)cc2Cl)[C@H](c2ccc(Cl)cc2)C1)c1ccc(F)cc1] |
| 903 | 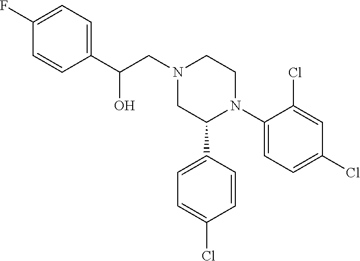 [SMILES: OC(CN1CCN(c2ccc(Cl)cc2Cl)[C@H](c2ccc(Cl)cc2)C1)c1ccc(F)cc1] |
| 904 | 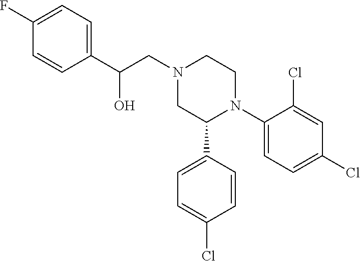 [SMILES: OC(CN1CCN(c2ccc(Cl)cc2Cl)[C@H](c2ccc(Cl)cc2)C1)c1ccc(F)cc1] |
| 905 | 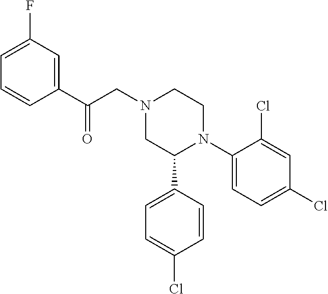 [SMILES: O=C(CN1CCN(c2ccc(Cl)cc2Cl)[C@H](c2ccc(Cl)cc2)C1)c1cccc(F)c1] |
| 906 | 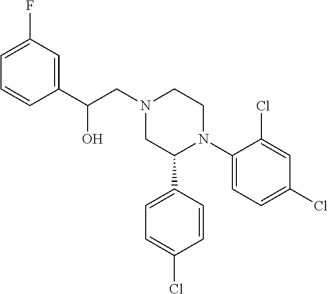 [SMILES: OC(CN1CCN(c2ccc(Cl)cc2Cl)[C@H](c2ccc(Cl)cc2)C1)c1cccc(F)c1] |
| 907 | 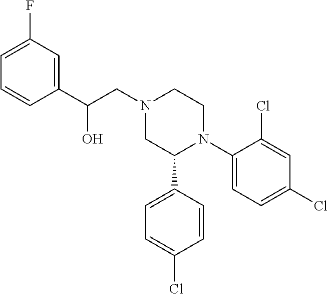 [SMILES: OC(CN1CCN(c2ccc(Cl)cc2Cl)[C@H](c2ccc(Cl)cc2)C1)c1cccc(F)c1] |
| 908 | 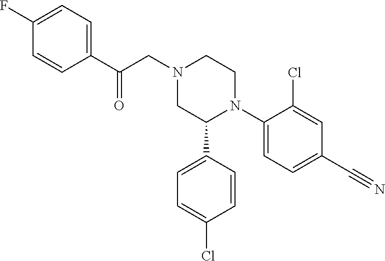 [SMILES: N#Cc1ccc(N2CCN(CC(=O)c3ccc(F)cc3)C[C@H]2c2ccc(Cl)cc2)c(Cl)c1] |
| 909 | 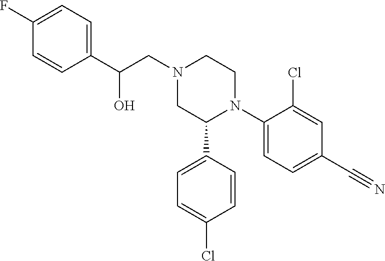 [SMILES: N#Cc1ccc(N2CCN(CC(O)c3ccc(F)cc3)C[C@H]2c2ccc(Cl)cc2)c(Cl)c1] |
| 910 | 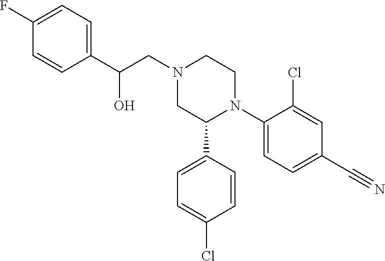 [SMILES: N#Cc1ccc(N2CCN(CC(O)c3ccc(F)cc3)C[C@H]2c2ccc(Cl)cc2)c(Cl)c1] |
| 911 | 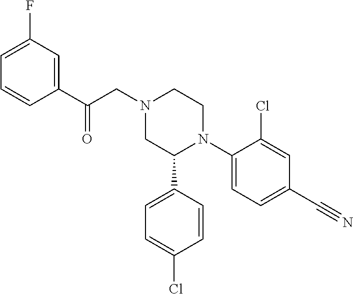 [SMILES: N#Cc1ccc(N2CCN(CC(=O)c3cccc(F)c3)C[C@H]2c2ccc(Cl)cc2)c(Cl)c1] |
| 912 | 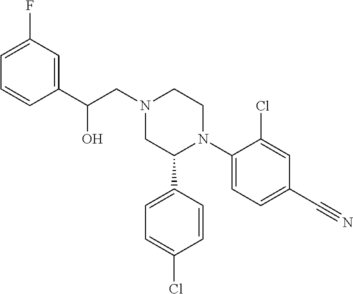 [SMILES: N#Cc1ccc(N2CCN(CC(O)c3cccc(F)c3)C[C@H]2c2ccc(Cl)cc2)c(Cl)c1] |
| 913 | 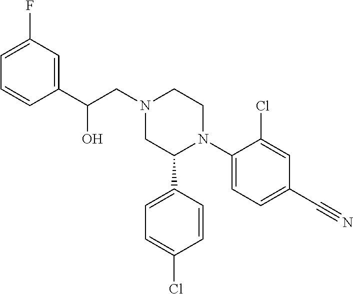 [SMILES: N#Cc1ccc(N2CCN(CC(O)c3cccc(F)c3)C[C@H]2c2ccc(Cl)cc2)c(Cl)c1] |
| 915 | 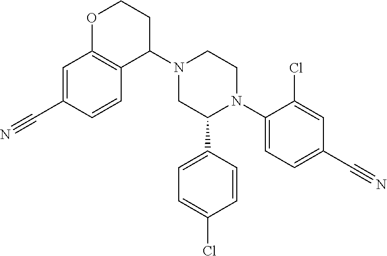 [SMILES: N#Cc1ccc(N2CCN(C3CCOc4cc(C#N)ccc43)C[C@H]2c2ccc(Cl)cc2)c(Cl)c1] |
| 916 | 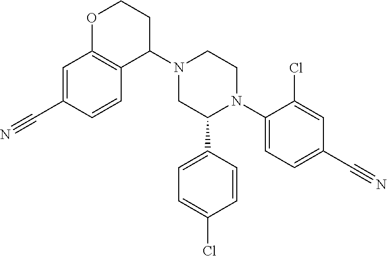 [SMILES: N#Cc1ccc(N2CCN(C3CCOc4cc(C#N)ccc43)C[C@H]2c2ccc(Cl)cc2)c(Cl)c1] |
| 918 | 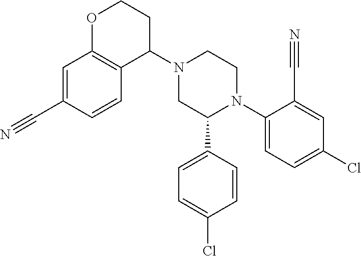 [SMILES: N#Cc1ccc2c(c1)OCCC2N1CCN(c2ccc(Cl)cc2C#N)[C@H](c2ccc(Cl)cc2)C1] |
| 919 | 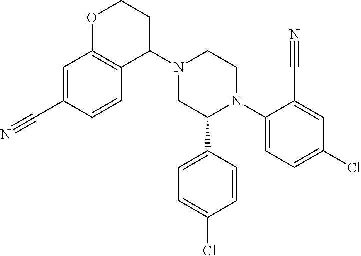 [SMILES: N#Cc1ccc2c(c1)OCCC2N1CCN(c2ccc(Cl)cc2C#N)[C@H](c2ccc(Cl)cc2)C1] |
| 920 | 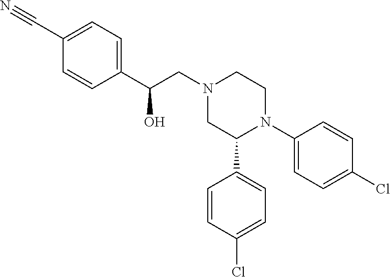 [SMILES: N#Cc1ccc([C@H](O)CN2CCN(c3ccc(Cl)cc3)[C@H](c3ccc(Cl)cc3)C2)cc1] |
| 921 | 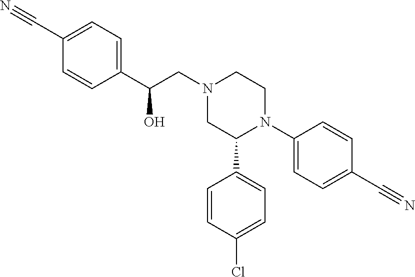 [SMILES: N#Cc1ccc([C@H](O)CN2CCN(c3ccc(C#N)cc3)[C@H](c3ccc(Cl)cc3)C2)cc1] |
| 922 | 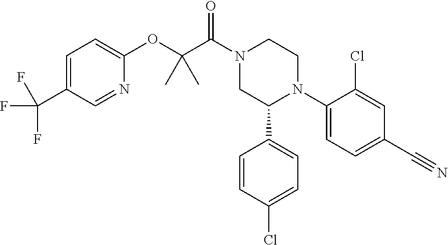 [SMILES: CC(C)(Oc1ccc(C(F)(F)F)cn1)C(=O)N1CCN(c2ccc(C#N)cc2Cl)[C@H](c2ccc(Cl)cc2)C1] |
| 923 | 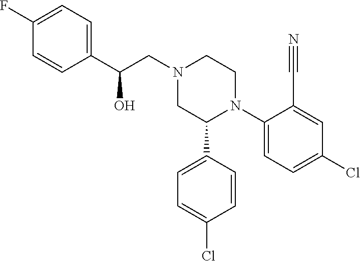 [SMILES: N#Cc1cc(Cl)ccc1N1CCN(C[C@@H](O)c2ccc(F)cc2)C[C@H]1c1ccc(Cl)cc1] |
| 923a | 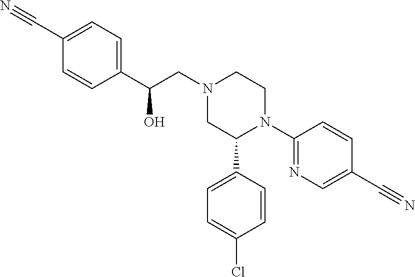 [SMILES: N#Cc1ccc([C@H](O)CN2CCN(c3ccc(C#N)cn3)[C@H](c3ccc(Cl)cc3)C2)cc1] |
| 923b | 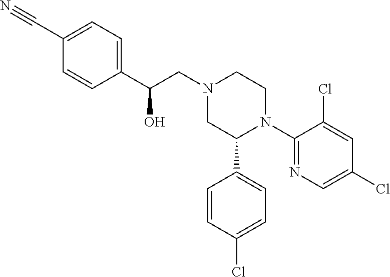 [SMILES: N#Cc1ccc([C@H](O)CN2CCN(c3ncc(Cl)cc3Cl)[C@H](c3ccc(Cl)cc3)C2)cc1] |
| 923c | 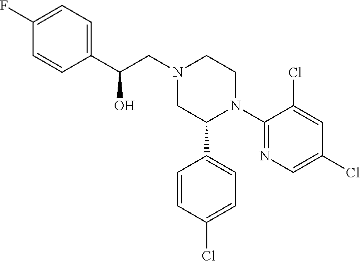 [SMILES: O[C@H](CN1CCN(c2ncc(Cl)cc2Cl)[C@H](c2ccc(Cl)cc2)C1)c1ccc(F)cc1] |
| 924 | 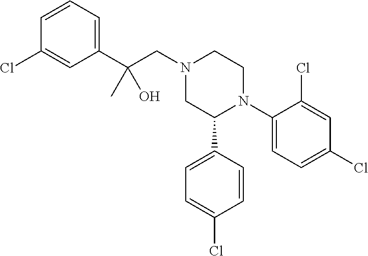 [SMILES: CC(O)(CN1CCN(c2ccc(Cl)cc2Cl)[C@H](c2ccc(Cl)cc2)C1)c1cccc(Cl)c1] |
| 925 | 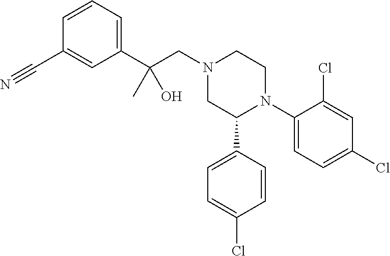 [SMILES: CC(O)(CN1CCN(c2ccc(Cl)cc2Cl)[C@H](c2ccc(Cl)cc2)C1)c1cccc(C#N)c1] |
| 926 | 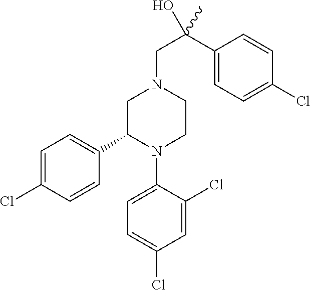 [SMILES: CC(O)(CN1CCN(c2ccc(Cl)cc2Cl)[C@H](c2ccc(Cl)cc2)C1)c1ccc(Cl)cc1] |
| 927 | 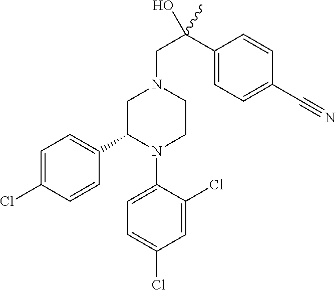 [SMILES: CC(O)(CN1CCN(c2ccc(Cl)cc2Cl)[C@H](c2ccc(Cl)cc2)C1)c1ccc(C#N)cc1] |
| 928 | 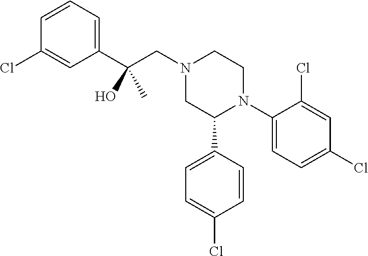 [SMILES: C[C@@](O)(CN1CCN(c2ccc(Cl)cc2Cl)[C@H](c2ccc(Cl)cc2)C1)c1cccc(Cl)c1] |
| 929 | 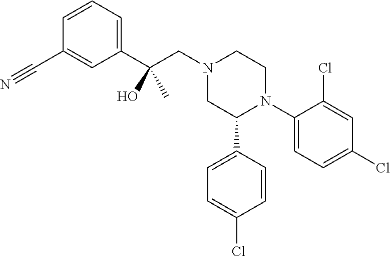 [SMILES: C[C@@](O)(CN1CCN(c2ccc(Cl)cc2Cl)[C@H](c2ccc(Cl)cc2)C1)c1cccc(C#N)c1] |
| 930 | 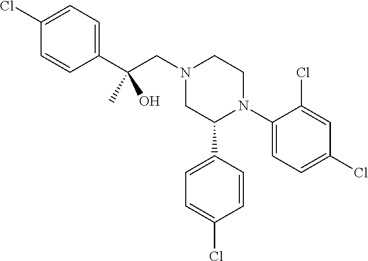 [SMILES: C[C@@](O)(CN1CCN(c2ccc(Cl)cc2Cl)[C@H](c2ccc(Cl)cc2)C1)c1ccc(Cl)cc1] |
| 931 | 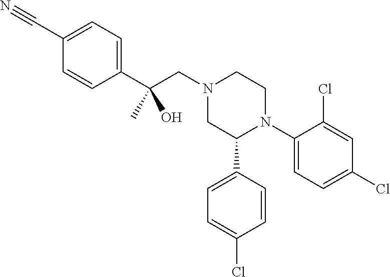 [SMILES: C[C@@](O)(CN1CCN(c2ccc(Cl)cc2Cl)[C@H](c2ccc(Cl)cc2)C1)c1ccc(C#N)cc1] |
| 932 | 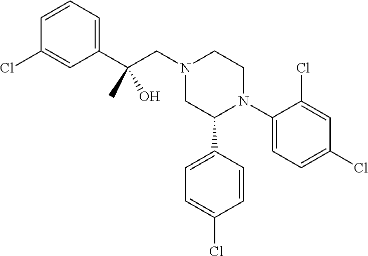 [SMILES: C[C@](O)(CN1CCN(c2ccc(Cl)cc2Cl)[C@H](c2ccc(Cl)cc2)C1)c1cccc(Cl)c1] |
| 933 | 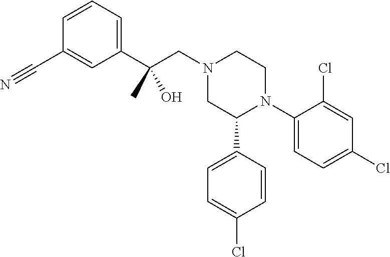 [SMILES: C[C@](O)(CN1CCN(c2ccc(Cl)cc2Cl)[C@H](c2ccc(Cl)cc2)C1)c1cccc(C#N)c1] |
| 934 | 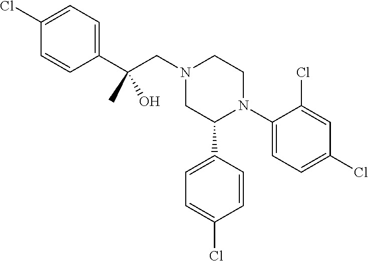 [SMILES: C[C@](O)(CN1CCN(c2ccc(Cl)cc2Cl)[C@H](c2ccc(Cl)cc2)C1)c1ccc(Cl)cc1] |
| 935 | 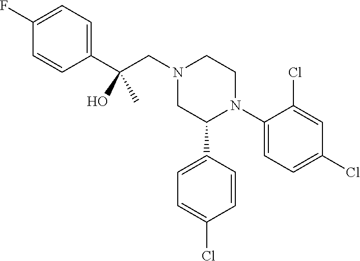 [SMILES: C[C@@](O)(CN1CCN(c2ccc(Cl)cc2Cl)[C@H](c2ccc(Cl)cc2)C1)c1ccc(F)cc1] |
| 936 | 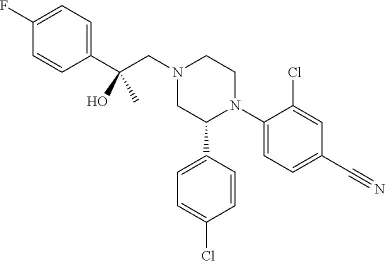 [SMILES: C[C@@](O)(CN1CCN(c2ccc(C#N)cc2Cl)[C@H](c2ccc(Cl)cc2)C1)c1ccc(F)cc1] |
| 937 | 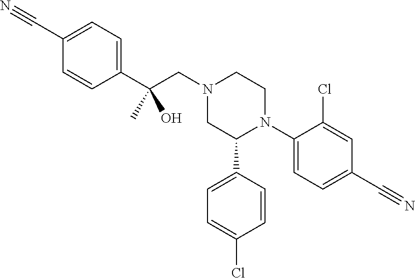 [SMILES: C[C@@](O)(CN1CCN(c2ccc(C#N)cc2Cl)[C@H](c2ccc(Cl)cc2)C1)c1ccc(C#N)cc1] |
| 938 | 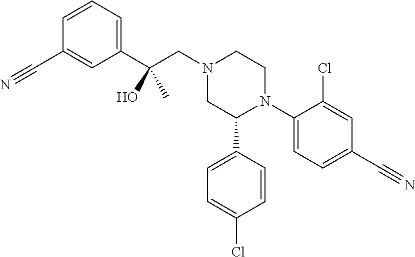 [SMILES: C[C@@](O)(CN1CCN(c2ccc(C#N)cc2Cl)[C@H](c2ccc(Cl)cc2)C1)c1cccc(C#N)c1] |
| 939 | 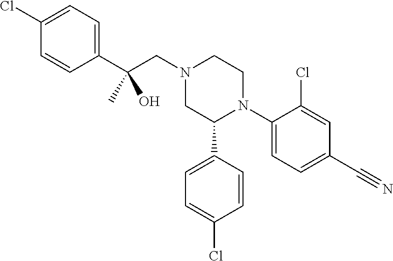 [SMILES: C[C@@](O)(CN1CCN(c2ccc(C#N)cc2Cl)[C@H](c2ccc(Cl)cc2)C1)c1ccc(Cl)cc1] |
| 940 | 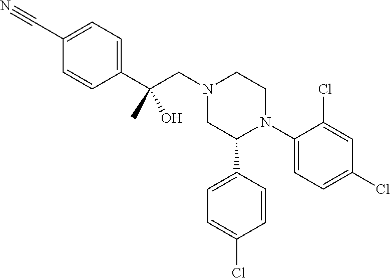 [SMILES: C[C@](O)(CN1CCN(c2ccc(Cl)cc2Cl)[C@H](c2ccc(Cl)cc2)C1)c1ccc(C#N)cc1] |
| 941 | 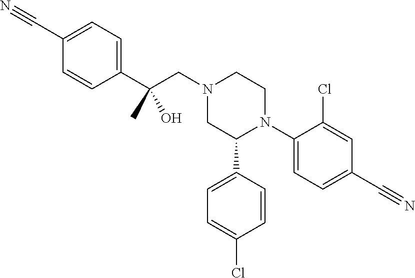 [SMILES: C[C@](O)(CN1CCN(c2ccc(C#N)cc2Cl)[C@H](c2ccc(Cl)cc2)C1)c1ccc(C#N)cc1] |
| 942 | 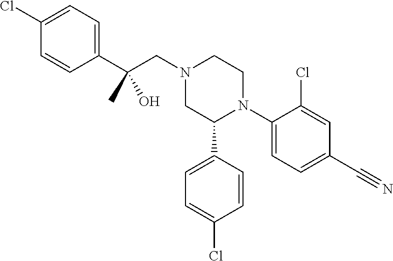 [SMILES: C[C@](O)(CN1CCN(c2ccc(C#N)cc2Cl)[C@H](c2ccc(Cl)cc2)C1)c1ccc(Cl)cc1] |
| 943 | 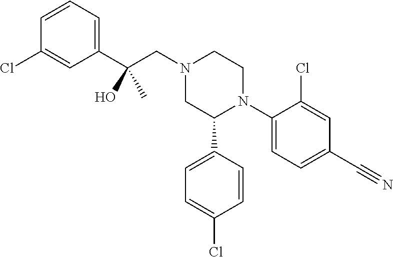 [SMILES: C[C@@](O)(CN1CCN(c2ccc(C#N)cc2Cl)[C@H](c2ccc(Cl)cc2)C1)c1cccc(Cl)c1] |
| 944 | 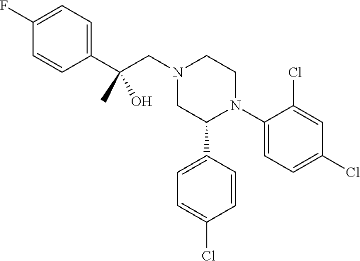 [SMILES: C[C@](O)(CN1CCN(c2ccc(Cl)cc2Cl)[C@H](c2ccc(Cl)cc2)C1)c1ccc(F)cc1] |
| 945 | 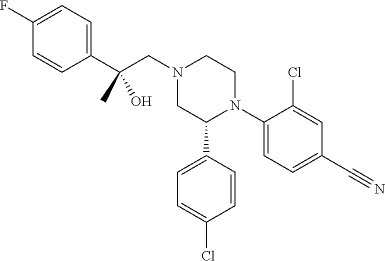 [SMILES: C[C@](O)(CN1CCN(c2ccc(C#N)cc2Cl)[C@H](c2ccc(Cl)cc2)C1)c1ccc(F)cc1] |
| 946 | 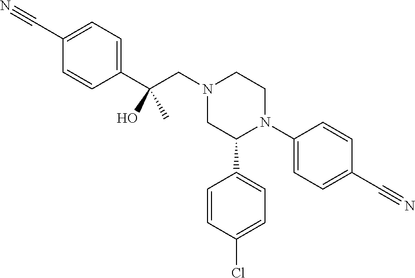 [SMILES: C[C@@](O)(CN1CCN(c2ccc(C#N)cc2)[C@H](c2ccc(Cl)cc2)C1)c1ccc(C#N)cc1] |
| 947 | 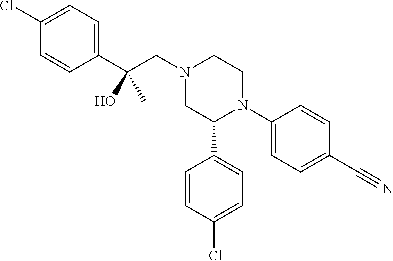 [SMILES: C[C@@](O)(CN1CCN(c2ccc(C#N)cc2)[C@H](c2ccc(Cl)cc2)C1)c1ccc(Cl)cc1] |
| 948 | 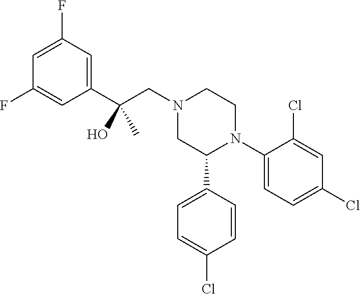 [SMILES: C[C@@](O)(CN1CCN(c2ccc(Cl)cc2Cl)[C@H](c2ccc(Cl)cc2)C1)c1cc(F)cc(F)c1] |
| 949 | 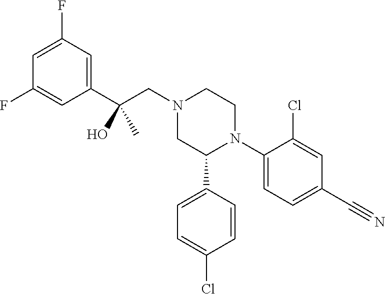 [SMILES: C[C@@](O)(CN1CCN(c2ccc(C#N)cc2Cl)[C@H](c2ccc(Cl)cc2)C1)c1cc(F)cc(F)c1] |
| 950 | 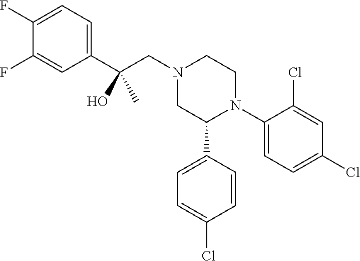 [SMILES: C[C@@](O)(CN1CCN(c2ccc(Cl)cc2Cl)[C@H](c2ccc(Cl)cc2)C1)c1ccc(F)c(F)c1] |
| 951 | 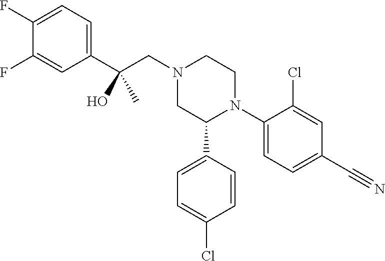 [SMILES: C[C@@](O)(CN1CCN(c2ccc(C#N)cc2Cl)[C@H](c2ccc(Cl)cc2)C1)c1ccc(F)c(F)c1] |
| 952 | 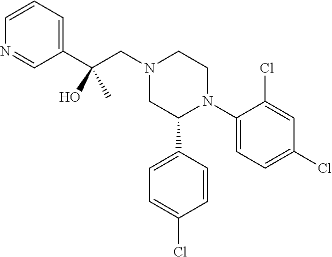 [SMILES: C[C@@](O)(CN1CCN(c2ccc(Cl)cc2Cl)[C@H](c2ccc(Cl)cc2)C1)c1cccnc1] |
| 953 | 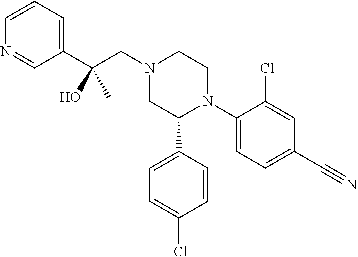 [SMILES: C[C@@](O)(CN1CCN(c2ccc(C#N)cc2Cl)[C@H](c2ccc(Cl)cc2)C1)c1cccnc1] |
| 954 | 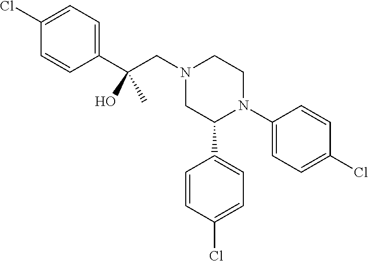 [SMILES: C[C@@](O)(CN1CCN(c2ccc(Cl)cc2)[C@H](c2ccc(Cl)cc2)C1)c1ccc(Cl)cc1] |
| 955 | 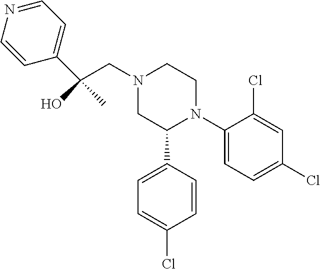 [SMILES: C[C@@](O)(CN1CCN(c2ccc(Cl)cc2Cl)[C@H](c2ccc(Cl)cc2)C1)c1ccncc1] |
| 956 | 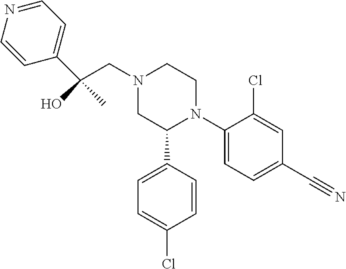 [SMILES: C[C@@](O)(CN1CCN(c2ccc(C#N)cc2Cl)[C@H](c2ccc(Cl)cc2)C1)c1ccncc1] |
| 957 | 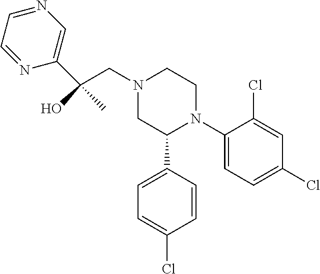 [SMILES: C[C@@](O)(CN1CCN(c2ccc(Cl)cc2Cl)[C@H](c2ccc(Cl)cc2)C1)c1cnccn1] |
| 958 | 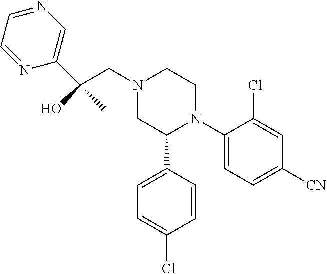 [SMILES: C[C@@](O)(CN1CCN(c2ccc(C#N)cc2Cl)[C@H](c2ccc(Cl)cc2)C1)c1cnccn1] |
| 959 | 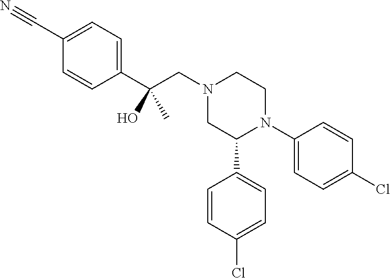 [SMILES: C[C@@](O)(CN1CCN(c2ccc(Cl)cc2)[C@H](c2ccc(Cl)cc2)C1)c1ccc(C#N)cc1] |
| 960 | 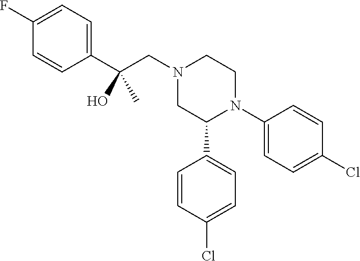 [SMILES: C[C@@](O)(CN1CCN(c2ccc(Cl)cc2)[C@H](c2ccc(Cl)cc2)C1)c1ccc(F)cc1] |
| 961 | 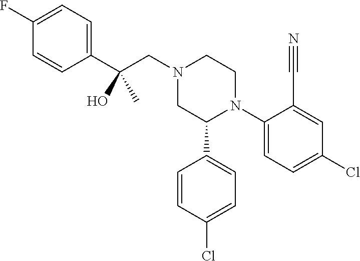 [SMILES: C[C@@](O)(CN1CCN(c2ccc(Cl)cc2C#N)[C@H](c2ccc(Cl)cc2)C1)c1ccc(F)cc1] |
| 962 | 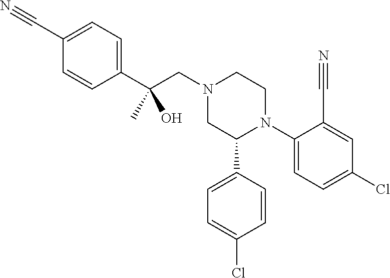 [SMILES: C[C@@](O)(CN1CCN(c2ccc(Cl)cc2C#N)[C@H](c2ccc(Cl)cc2)C1)c1ccc(C#N)cc1] |
| 963 | 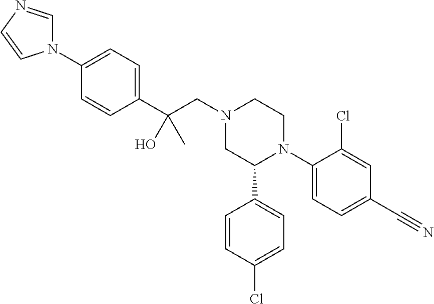 [SMILES: CC(O)(CN1CCN(c2ccc(C#N)cc2Cl)[C@H](c2ccc(Cl)cc2)C1)c1ccc(-n2ccnc2)cc1] |
| 964 | 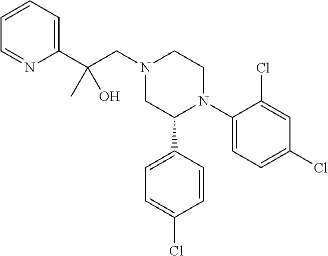 [SMILES: CC(O)(CN1CCN(c2ccc(Cl)cc2Cl)[C@H](c2ccc(Cl)cc2)C1)c1ccccn1] |
| 965a | 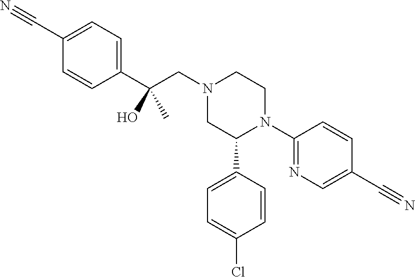 [SMILES: C[C@@](O)(CN1CCN(c2ccc(C#N)cn2)[C@H](c2ccc(Cl)cc2)C1)c1ccc(C#N)cc1] |
| 965b | 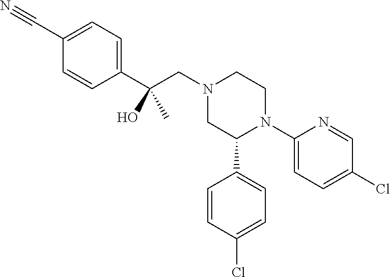 [SMILES: C[C@@](O)(CN1CCN(c2ccc(Cl)cn2)[C@H](c2ccc(Cl)cc2)C1)c1ccc(C#N)cc1] |
| 965c | 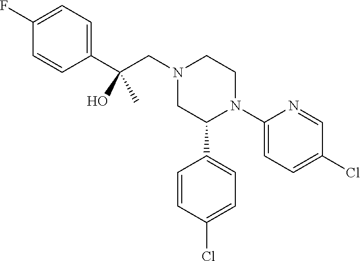 [SMILES: C[C@@](O)(CN1CCN(c2ccc(Cl)cn2)[C@H](c2ccc(Cl)cc2)C1)c1ccc(F)cc1] |
| 965d | 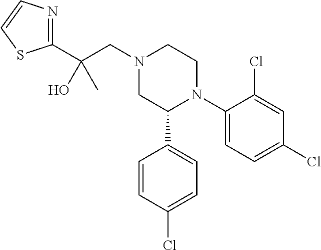 [SMILES: CC(O)(CN1CCN(c2ccc(Cl)cc2Cl)[C@H](c2ccc(Cl)cc2)C1)c1nccs1] |
| 965e | 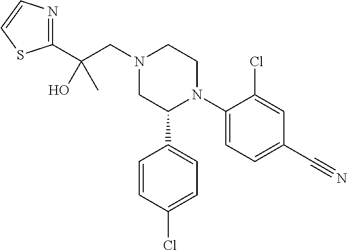 [SMILES: CC(O)(CN1CCN(c2ccc(C#N)cc2Cl)[C@H](c2ccc(Cl)cc2)C1)c1nccs1] |
| 965f | 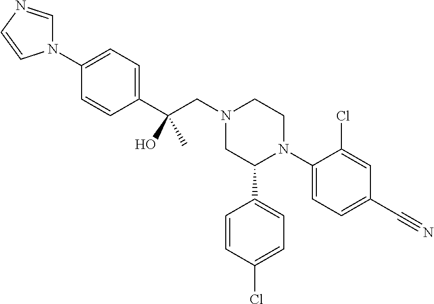 [SMILES: C[C@@](O)(CN1CCN(c2ccc(C#N)cc2Cl)[C@H](c2ccc(Cl)cc2)C1)c1ccc(-n2ccnc2)cc1] |
| 966 | 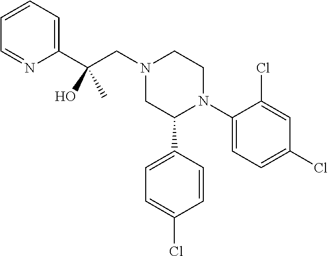 [SMILES: C[C@@](O)(CN1CCN(c2ccc(Cl)cc2Cl)[C@H](c2ccc(Cl)cc2)C1)c1ccccn1] |
| 967 | 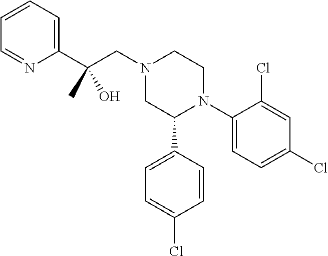 [SMILES: C[C@](O)(CN1CCN(c2ccc(Cl)cc2Cl)[C@H](c2ccc(Cl)cc2)C1)c1ccccn1] |
| 968 | 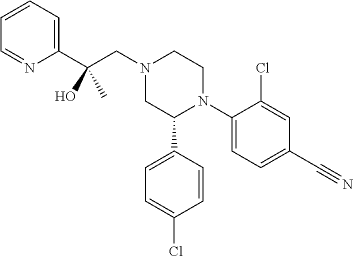 [SMILES: C[C@@](O)(CN1CCN(c2ccc(C#N)cc2Cl)[C@H](c2ccc(Cl)cc2)C1)c1ccccn1] |
| 969 | 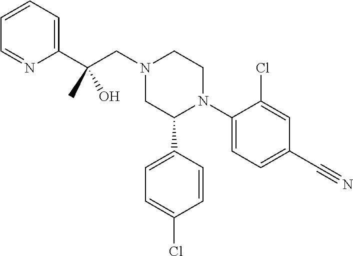 [SMILES: C[C@](O)(CN1CCN(c2ccc(C#N)cc2Cl)[C@H](c2ccc(Cl)cc2)C1)c1ccccn1] |
| 970 | 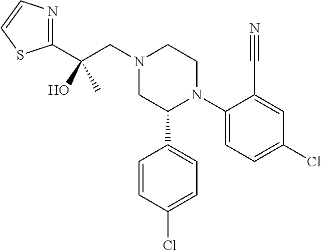 [SMILES: C[C@@](O)(CN1CCN(c2ccc(Cl)cc2C#N)[C@H](c2ccc(Cl)cc2)C1)c1nccs1] |
| 971 | 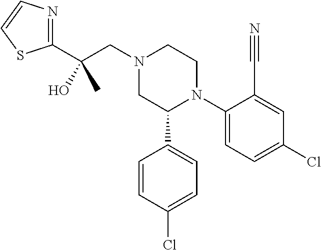 [SMILES: C[C@](O)(CN1CCN(c2ccc(Cl)cc2C#N)[C@H](c2ccc(Cl)cc2)C1)c1nccs1] |
| 972 | 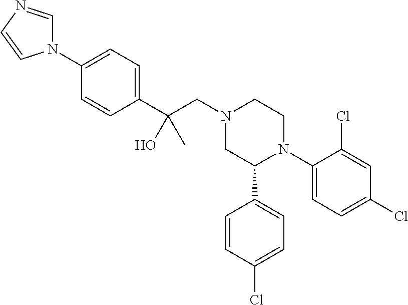 [SMILES: CC(O)(CN1CCN(c2ccc(Cl)cc2Cl)[C@H](c2ccc(Cl)cc2)C1)c1ccc(-n2ccnc2)cc1] |
| 973 | 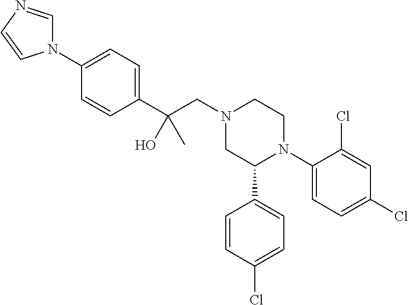 [SMILES: CC(O)(CN1CCN(c2ccc(Cl)cc2Cl)[C@H](c2ccc(Cl)cc2)C1)c1ccc(-n2ccnc2)cc1] |
| 974 | 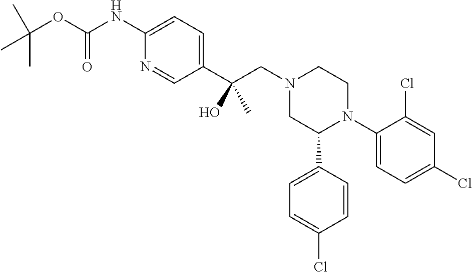 [SMILES: CC(C)(C)OC(=O)Nc1ccc([C@](C)(O)CN2CCN(c3ccc(Cl)cc3Cl)[C@H](c3ccc(Cl)cc3)C2)cn1] |
| 975 | 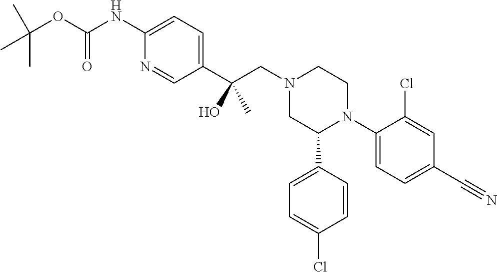 [SMILES: CC(C)(C)OC(=O)Nc1ccc([C@](C)(O)CN2CCN(c3ccc(C#N)cc3Cl)[C@H](c3ccc(Cl)cc3)C2)cn1] |
| 976 | 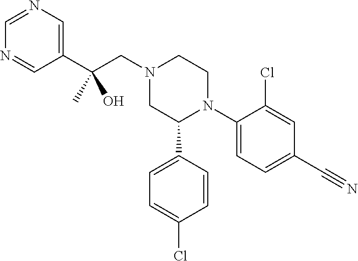 [SMILES: C[C@@](O)(CN1CCN(c2ccc(C#N)cc2Cl)[C@H](c2ccc(Cl)cc2)C1)c1cncnc1] |
| 977 | 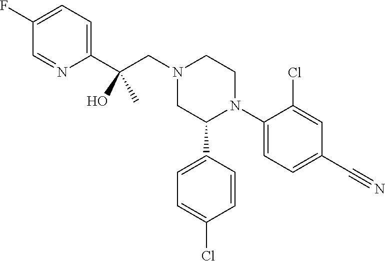 [SMILES: C[C@@](O)(CN1CCN(c2ccc(C#N)cc2Cl)[C@H](c2ccc(Cl)cc2)C1)c1ccc(F)cn1] |
| 978 | 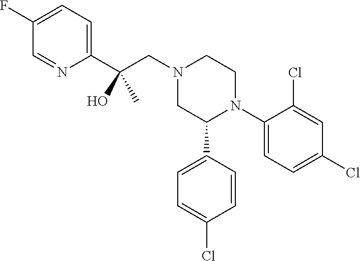 [SMILES: C[C@@](O)(CN1CCN(c2ccc(Cl)cc2Cl)[C@H](c2ccc(Cl)cc2)C1)c1ccc(F)cn1] |
| 979 | 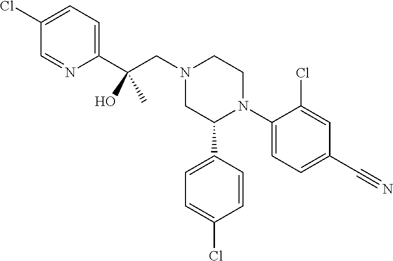 [SMILES: C[C@@](O)(CN1CCN(c2ccc(C#N)cc2Cl)[C@H](c2ccc(Cl)cc2)C1)c1ccc(Cl)cn1] |
| 980 | 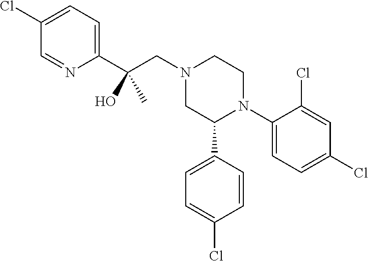 [SMILES: C[C@@](O)(CN1CCN(c2ccc(Cl)cc2Cl)[C@H](c2ccc(Cl)cc2)C1)c1ccc(Cl)cn1] |
| 981 | 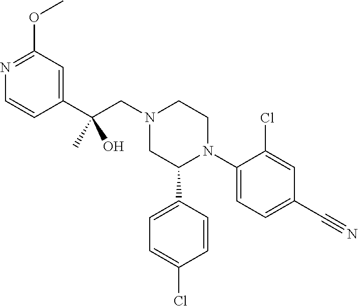 [SMILES: COc1cc([C@](C)(O)CN2CCN(c3ccc(C#N)cc3Cl)[C@H](c3ccc(Cl)cc3)C2)ccn1] |
| 982 | 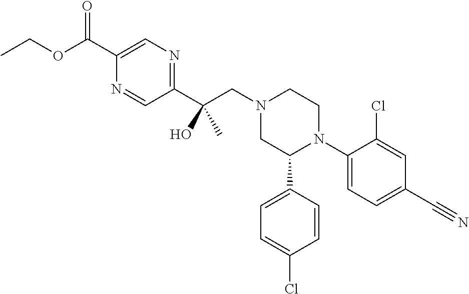 [SMILES: CCOC(=O)c1cnc([C@](C)(O)CN2CCN(c3ccc(C#N)cc3Cl)[C@H](c3ccc(Cl)cc3)C2)cn1] |
| 983 | 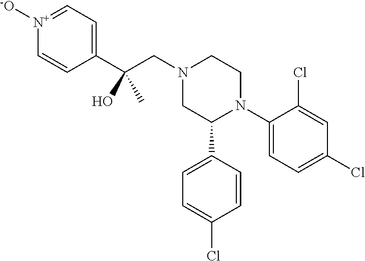 |
| 984 | 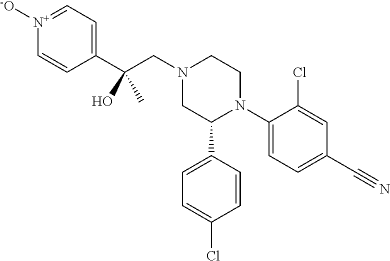 |
| 985 | 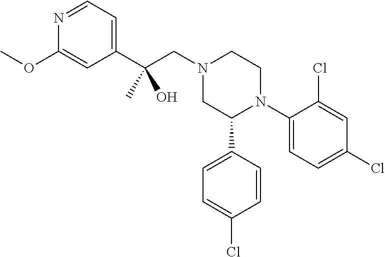 [SMILES: COc1cc([C@](C)(O)CN2CCN(c3ccc(Cl)cc3Cl)[C@H](c3ccc(Cl)cc3)C2)ccn1] |
| 986 | 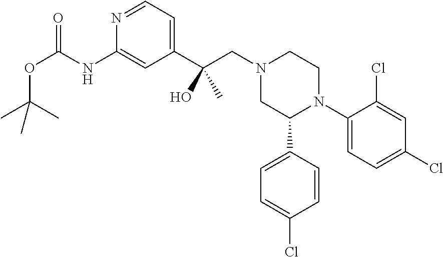 [SMILES: CC(C)(C)OC(=O)Nc1cc([C@](C)(O)CN2CCN(c3ccc(Cl)cc3Cl)[C@H](c3ccc(Cl)cc3)C2)ccn1] |
| 987 | 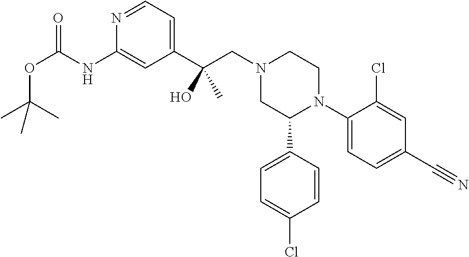 [SMILES: CC(C)(C)OC(=O)Nc1cc([C@](C)(O)CN2CCN(c3ccc(C#N)cc3Cl)[C@H](c3ccc(Cl)cc3)C2)ccn1] |
| 988 | 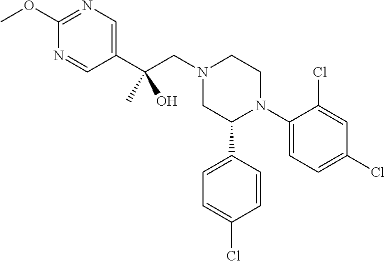 [SMILES: COc1ncc([C@](C)(O)CN2CCN(c3ccc(Cl)cc3Cl)[C@H](c3ccc(Cl)cc3)C2)cn1] |
| 989 | 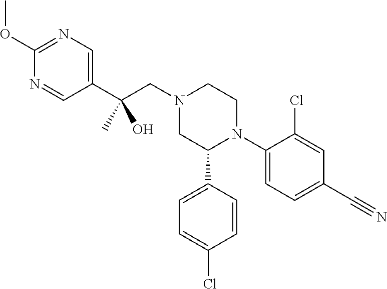 [SMILES: COc1ncc([C@](C)(O)CN2CCN(c3ccc(C#N)cc3Cl)[C@H](c3ccc(Cl)cc3)C2)cn1] |
| 990 | 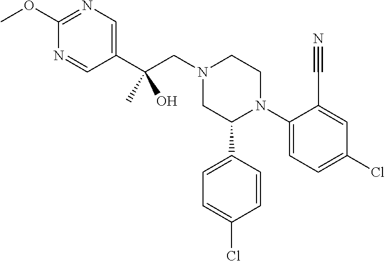 [SMILES: COc1ncc([C@](C)(O)CN2CCN(c3ccc(Cl)cc3C#N)[C@H](c3ccc(Cl)cc3)C2)cn1] |
| 991 | 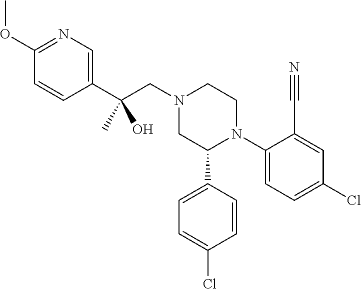 [SMILES: COc1ccc([C@](C)(O)CN2CCN(c3ccc(Cl)cc3C#N)[C@H](c3ccc(Cl)cc3)C2)cn1] |
| 992 | 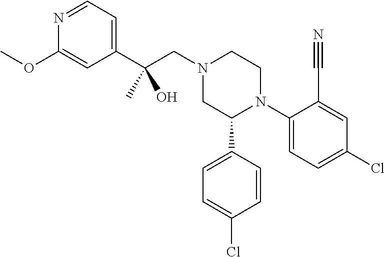 [SMILES: COc1cc([C@](C)(O)CN2CCN(c3ccc(Cl)cc3C#N)[C@H](c3ccc(Cl)cc3)C2)ccn1] |
| 993 | 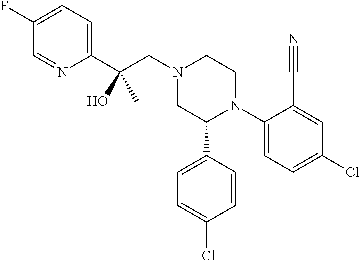 [SMILES: C[C@@](O)(CN1CCN(c2ccc(Cl)cc2C#N)[C@H](c2ccc(Cl)cc2)C1)c1ccc(F)cn1] |
| 994 | 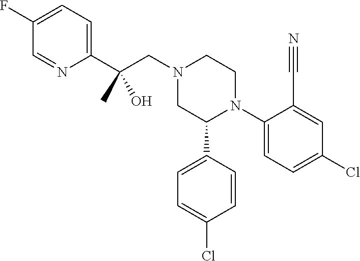 [SMILES: C[C@](O)(CN1CCN(c2ccc(Cl)cc2C#N)[C@H](c2ccc(Cl)cc2)C1)c1ccc(F)cn1] |
| 995 | 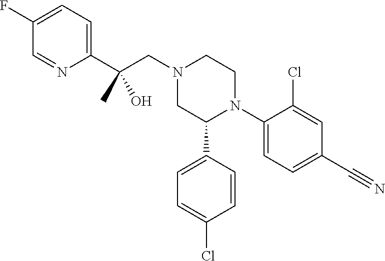 [SMILES: C[C@](O)(CN1CCN(c2ccc(C#N)cc2Cl)[C@H](c2ccc(Cl)cc2)C1)c1ccc(F)cn1] |
| 996 | 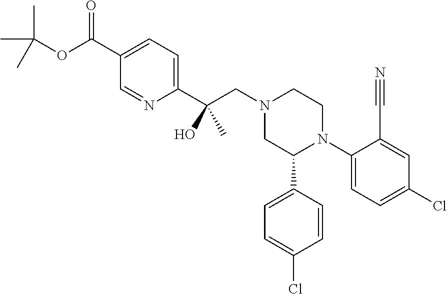 [SMILES: CC(C)(C)OC(=O)c1ccc([C@](C)(O)CN2CCN(c3ccc(Cl)cc3C#N)[C@H](c3ccc(Cl)cc3)C2)nc1] |
| 997 | 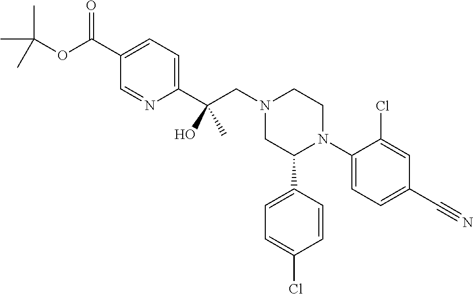 [SMILES: CC(C)(C)OC(=O)c1ccc([C@](C)(O)CN2CCN(c3ccc(C#N)cc3Cl)[C@H](c3ccc(Cl)cc3)C2)nc1] |
| 998 | 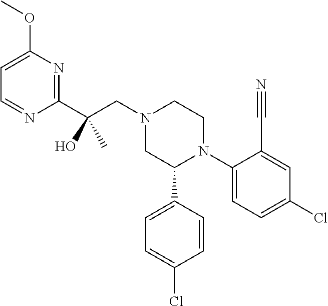 [SMILES: COc1ccnc([C@](C)(O)CN2CCN(c3ccc(Cl)cc3C#N)[C@H](c3ccc(Cl)cc3)C2)n1] |
| 999 | 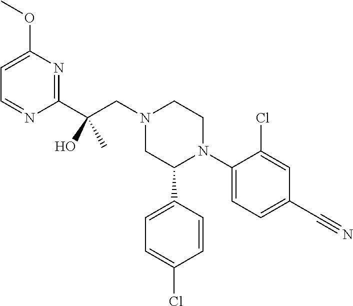 [SMILES: COc1ccnc([C@](C)(O)CN2CCN(c3ccc(C#N)cc3Cl)[C@H](c3ccc(Cl)cc3)C2)n1] |
| 1000 | 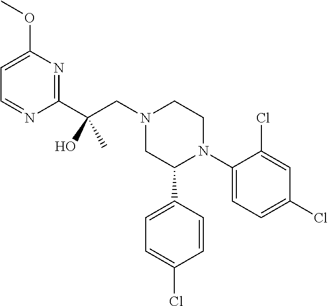 [SMILES: COc1ccnc([C@](C)(O)CN2CCN(c3ccc(Cl)cc3Cl)[C@H](c3ccc(Cl)cc3)C2)n1] |
| 1001 | 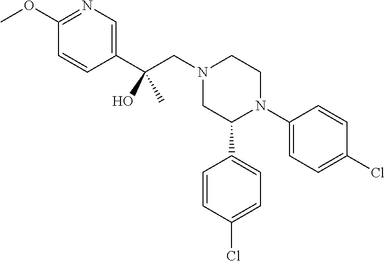 [SMILES: COc1ccc([C@](C)(O)CN2CCN(c3ccc(Cl)cc3)[C@H](c3ccc(Cl)cc3)C2)cn1] |
| 1002 | 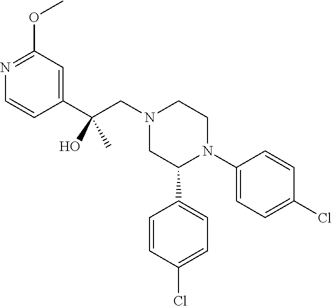 [SMILES: COc1cc([C@](C)(O)CN2CCN(c3ccc(Cl)cc3)[C@H](c3ccc(Cl)cc3)C2)ccn1] |
| 1003 | 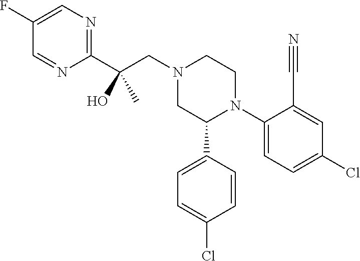 [SMILES: C[C@@](O)(CN1CCN(c2ccc(Cl)cc2C#N)[C@H](c2ccc(Cl)cc2)C1)c1ncc(F)cn1] |
| 1004 | 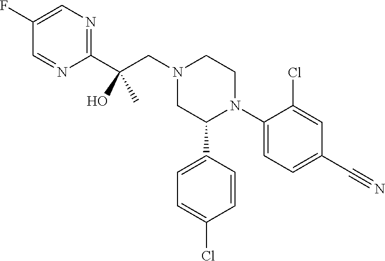 [SMILES: C[C@@](O)(CN1CCN(c2ccc(C#N)cc2Cl)[C@H](c2ccc(Cl)cc2)C1)c1ncc(F)cn1] |
| 1005 | 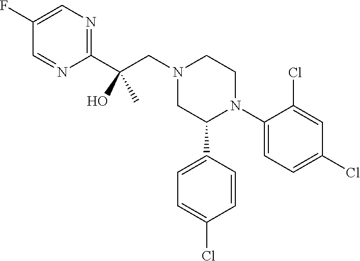 [SMILES: C[C@@](O)(CN1CCN(c2ccc(Cl)cc2Cl)[C@H](c2ccc(Cl)cc2)C1)c1ncc(F)cn1] |
| 1006 | 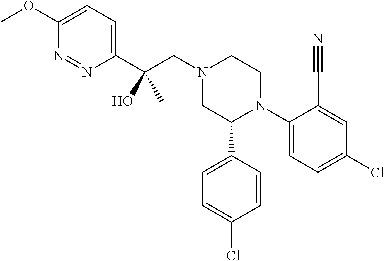 [SMILES: COc1ccc([C@](C)(O)CN2CCN(c3ccc(Cl)cc3C#N)[C@H](c3ccc(Cl)cc3)C2)nn1] |
| 1007 | 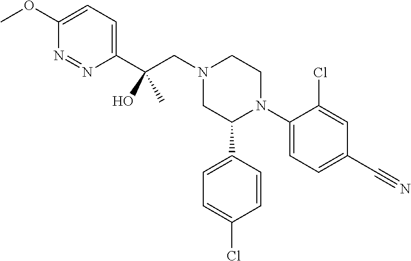 [SMILES: COc1ccc([C@](C)(O)CN2CCN(c3ccc(C#N)cc3Cl)[C@H](c3ccc(Cl)cc3)C2)nn1] |
| 1008 | 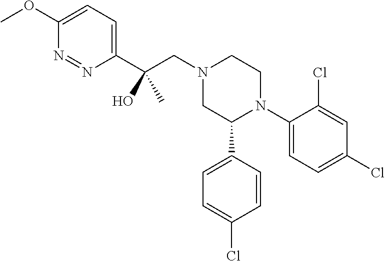 [SMILES: COc1ccc([C@](C)(O)CN2CCN(c3ccc(Cl)cc3Cl)[C@H](c3ccc(Cl)cc3)C2)nn1] |
| 1009 | 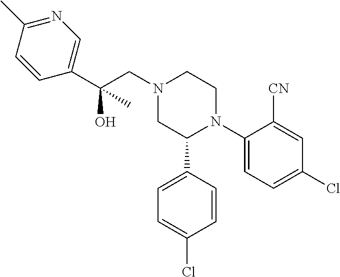 [SMILES: Cc1ccc([C@](C)(O)CN2CCN(c3ccc(Cl)cc3C#N)[C@H](c3ccc(Cl)cc3)C2)cn1] |
| 1010 | 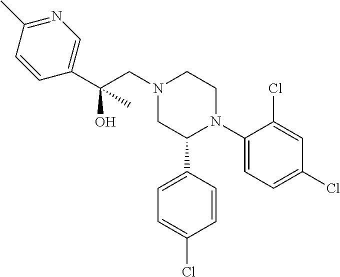 [SMILES: Cc1ccc([C@](C)(O)CN2CCN(c3ccc(Cl)cc3Cl)[C@H](c3ccc(Cl)cc3)C2)cn1] |
| 1011 | 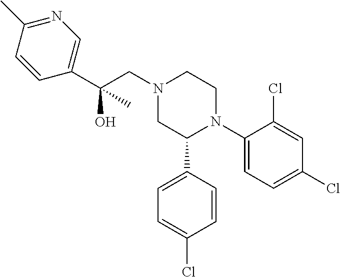 [SMILES: Cc1ccc([C@](C)(O)CN2CCN(c3ccc(Cl)cc3Cl)[C@H](c3ccc(Cl)cc3)C2)cn1] |
| 1012 | 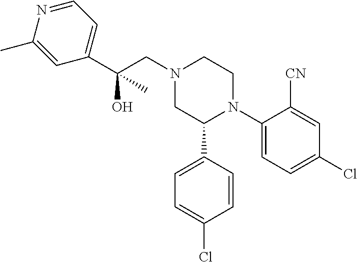 [SMILES: Cc1cc([C@](C)(O)CN2CCN(c3ccc(Cl)cc3C#N)[C@H](c3ccc(Cl)cc3)C2)ccn1] |
| 1013 | 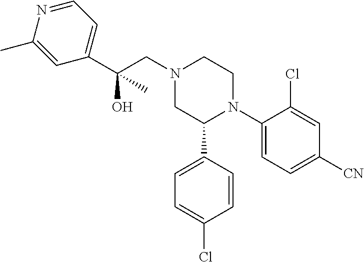 [SMILES: Cc1cc([C@](C)(O)CN2CCN(c3ccc(C#N)cc3Cl)[C@H](c3ccc(Cl)cc3)C2)ccn1] |
| 1014 | 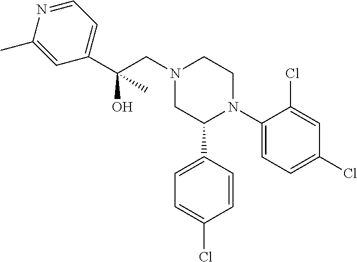 [SMILES: Cc1cc([C@](C)(O)CN2CCN(c3ccc(Cl)cc3Cl)[C@H](c3ccc(Cl)cc3)C2)ccn1] |
| 1015 | 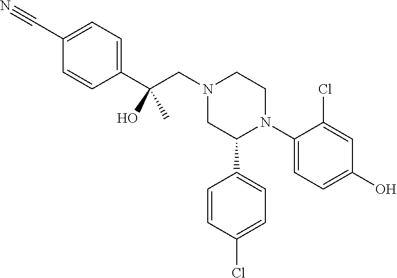 [SMILES: C[C@@](O)(CN1CCN(c2ccc(O)cc2Cl)[C@H](c2ccc(Cl)cc2)C1)c1ccc(C#N)cc1] |
| 1016 | 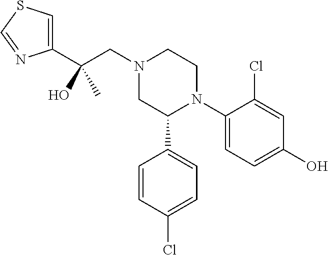 [SMILES: C[C@@](O)(CN1CCN(c2ccc(O)cc2Cl)[C@H](c2ccc(Cl)cc2)C1)c1cscn1] |
| 1017 | 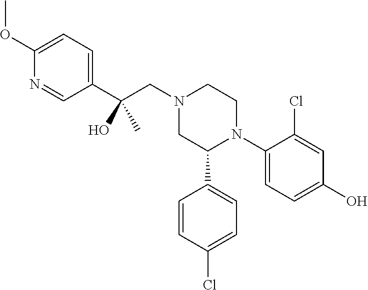 [SMILES: COc1ccc([C@](C)(O)CN2CCN(c3ccc(O)cc3Cl)[C@H](c3ccc(Cl)cc3)C2)cn1] |
| 1018 | 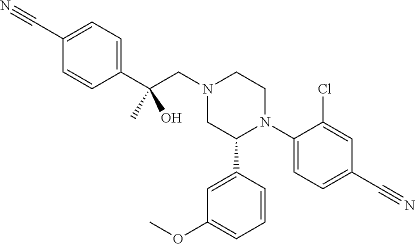 [SMILES: COc1cccc([C@@H]2CN(C[C@@](C)(O)c3ccc(C#N)cc3)CCN2c2ccc(C#N)cc2Cl)c1] |
| 1019 | 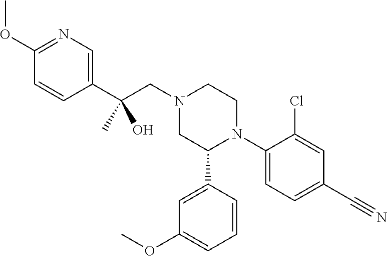 [SMILES: COc1cccc([C@@H]2CN(C[C@@](C)(O)c3ccc(OC)nc3)CCN2c2ccc(C#N)cc2Cl)c1] |
| 1020 | 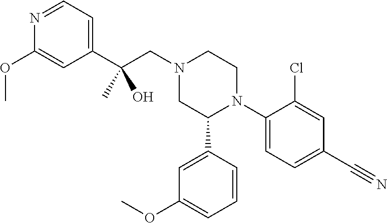 [SMILES: COc1cccc([C@@H]2CN(C[C@@](C)(O)c3ccnc(OC)c3)CCN2c2ccc(C#N)cc2Cl)c1] |
| 1021 | 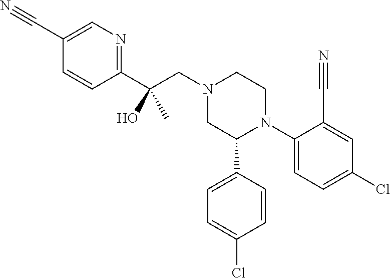 [SMILES: C[C@@](O)(CN1CCN(c2ccc(Cl)cc2C#N)[C@H](c2ccc(Cl)cc2)C1)c1ccc(C#N)cn1] |
| 1022 | 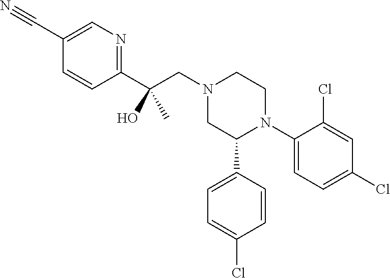 [SMILES: C[C@@](O)(CN1CCN(c2ccc(Cl)cc2Cl)[C@H](c2ccc(Cl)cc2)C1)c1ccc(C#N)cn1] |
| 1023 | 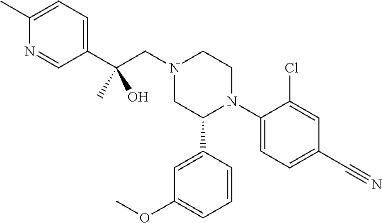 [SMILES: COc1cccc([C@@H]2CN(C[C@@](C)(O)c3ccc(C)nc3)CCN2c2ccc(C#N)cc2Cl)c1] |
| 1024 | 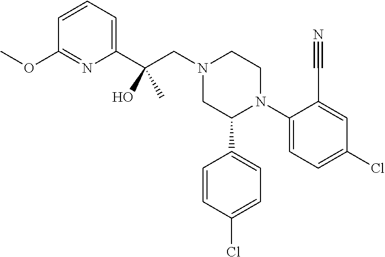 [SMILES: COc1cccc([C@](C)(O)CN2CCN(c3ccc(Cl)cc3C#N)[C@H](c3ccc(Cl)cc3)C2)n1] |
| 1025 | 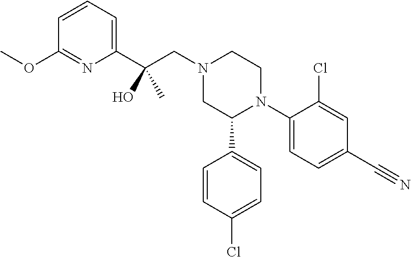 [SMILES: COc1cccc([C@](C)(O)CN2CCN(c3ccc(C#N)cc3Cl)[C@H](c3ccc(Cl)cc3)C2)n1] |
| 1026 | 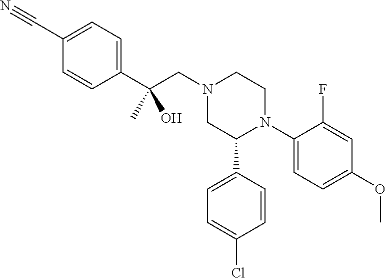 [SMILES: COc1ccc(N2CCN(C[C@@](C)(O)c3ccc(C#N)cc3)C[C@H]2c2ccc(Cl)cc2)c(F)c1] |
| 1027 | 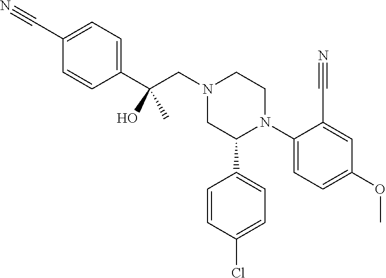 [SMILES: COc1ccc(N2CCN(C[C@@](C)(O)c3ccc(C#N)cc3)C[C@H]2c2ccc(Cl)cc2)c(C#N)c1] |
| 1028 | 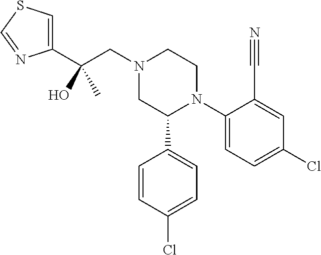 [SMILES: C[C@@](O)(CN1CCN(c2ccc(Cl)cc2C#N)[C@H](c2ccc(Cl)cc2)C1)c1cscn1] |
| 1029 | 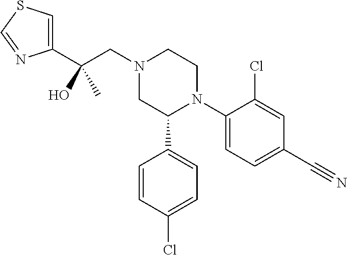 [SMILES: C[C@@](O)(CN1CCN(c2ccc(C#N)cc2Cl)[C@H](c2ccc(Cl)cc2)C1)c1cscn1] |
| 1030 | 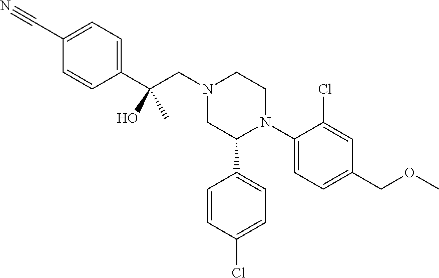 [SMILES: COCc1ccc(N2CCN(C[C@@](C)(O)c3ccc(C#N)cc3)C[C@H]2c2ccc(Cl)cc2)c(Cl)c1] |
| 1032 | 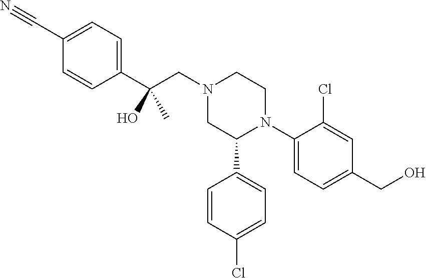 [SMILES: C[C@@](O)(CN1CCN(c2ccc(CO)cc2Cl)[C@H](c2ccc(Cl)cc2)C1)c1ccc(C#N)cc1] |
| 1033 | 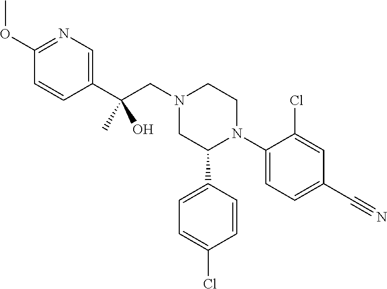 [SMILES: COc1ccc([C@](C)(O)CN2CCN(c3ccc(C#N)cc3Cl)[C@H](c3ccc(Cl)cc3)C2)cn1] |
| 1034 | 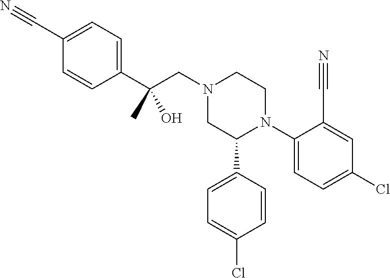 [SMILES: C[C@](O)(CN1CCN(c2ccc(Cl)cc2C#N)[C@H](c2ccc(Cl)cc2)C1)c1ccc(C#N)cc1] |
| 1035d | 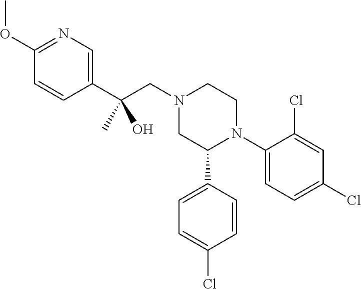 [SMILES: COc1ccc([C@](C)(O)CN2CCN(c3ccc(Cl)cc3Cl)[C@H](c3ccc(Cl)cc3)C2)cn1] |
| 1035e | 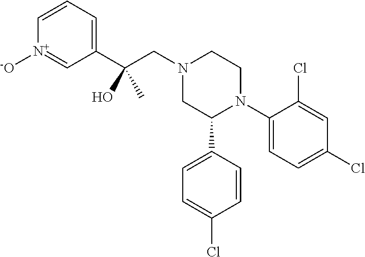 |
| 1035f | 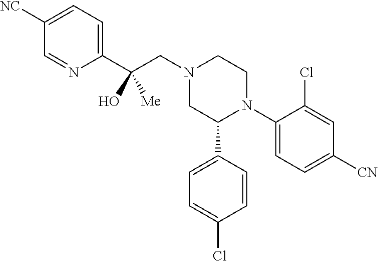 [SMILES: C[C@@](O)(CN1CCN(c2ccc(C#N)cc2Cl)[C@H](c2ccc(Cl)cc2)C1)c1ccc(C#N)cn1] |
| 1035 | 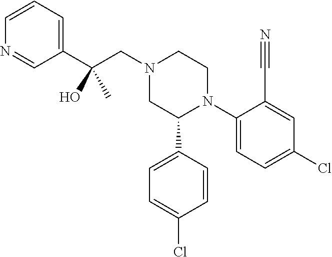 [SMILES: C[C@@](O)(CN1CCN(c2ccc(Cl)cc2C#N)[C@H](c2ccc(Cl)cc2)C1)c1cccnc1] |
| 1035g | 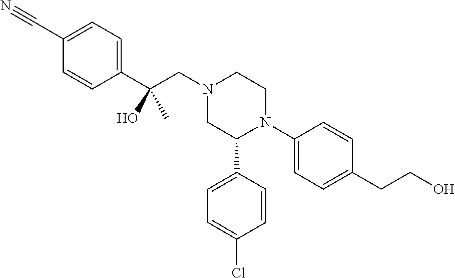 [SMILES: C[C@@](O)(CN1CCN(c2ccc(CCO)cc2)[C@H](c2ccc(Cl)cc2)C1)c1ccc(C#N)cc1] |
| 1035g | 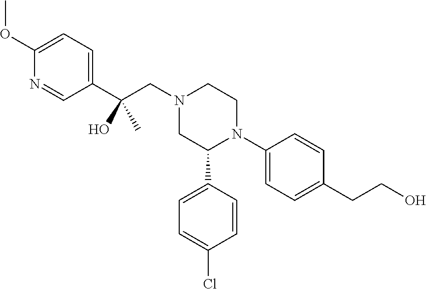 [SMILES: COc1ccc([C@](C)(O)CN2CCN(c3ccc(CCO)cc3)[C@H](c3ccc(Cl)cc3)C2)cn1] |
| 1035h | 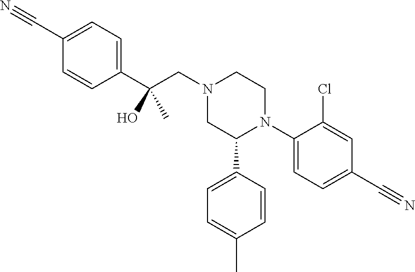 [SMILES: Cc1ccc([C@@H]2CN(C[C@@](C)(O)c3ccc(C#N)cc3)CCN2c2ccc(C#N)cc2Cl)cc1] |
| 1035a | 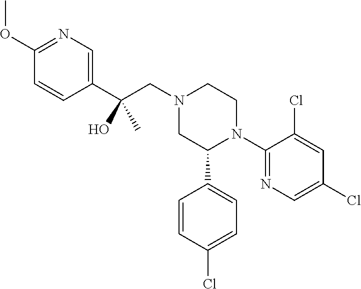 [SMILES: COc1ccc([C@](C)(O)CN2CCN(c3ncc(Cl)cc3Cl)[C@H](c3ccc(Cl)cc3)C2)cn1] |
| 1035c | 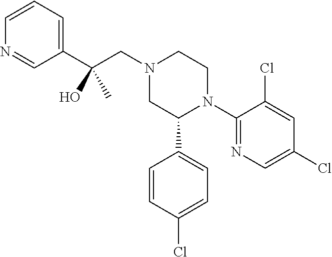 [SMILES: C[C@@](O)(CN1CCN(c2ncc(Cl)cc2Cl)[C@H](c2ccc(Cl)cc2)C1)c1cccnc1] |
| 1035b | 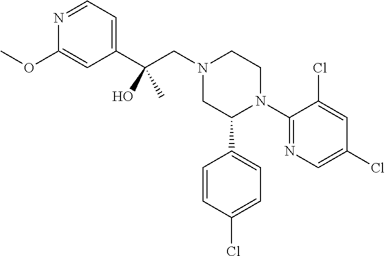 [SMILES: COc1cc([C@](C)(O)CN2CCN(c3ncc(Cl)cc3Cl)[C@H](c3ccc(Cl)cc3)C2)ccn1] |
| 1035i | 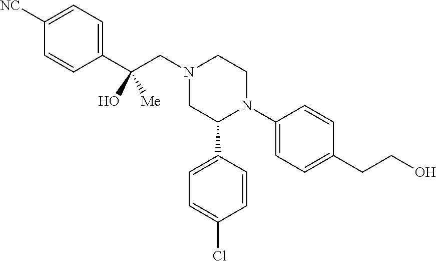 [SMILES: C[C@@](O)(CN1CCN(c2ccc(CCO)cc2)[C@H](c2ccc(Cl)cc2)C1)c1ccc(C#N)cc1] |
| 1036 | 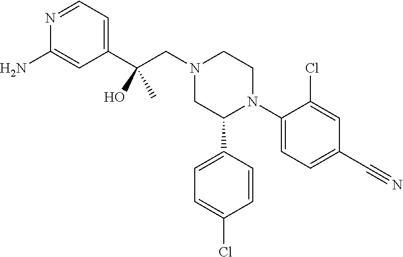 [SMILES: C[C@@](O)(CN1CCN(c2ccc(C#N)cc2Cl)[C@H](c2ccc(Cl)cc2)C1)c1ccnc(N)c1] |
| 1037 | 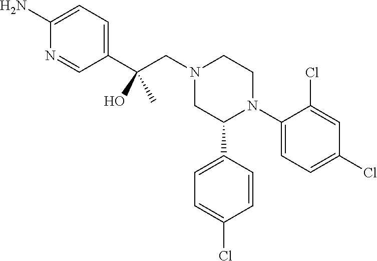 [SMILES: C[C@@](O)(CN1CCN(c2ccc(Cl)cc2Cl)[C@H](c2ccc(Cl)cc2)C1)c1ccc(N)nc1] |
| 1038 | 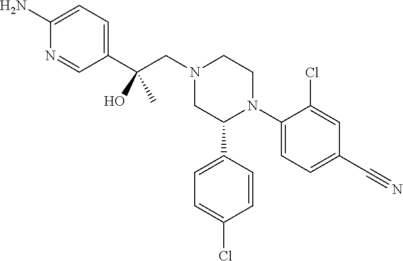 [SMILES: C[C@@](O)(CN1CCN(c2ccc(C#N)cc2Cl)[C@H](c2ccc(Cl)cc2)C1)c1ccc(N)nc1] |
| 1039 | 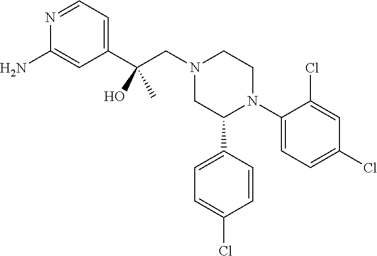 [SMILES: C[C@@](O)(CN1CCN(c2ccc(Cl)cc2Cl)[C@H](c2ccc(Cl)cc2)C1)c1ccnc(N)c1] |
| 1040 | 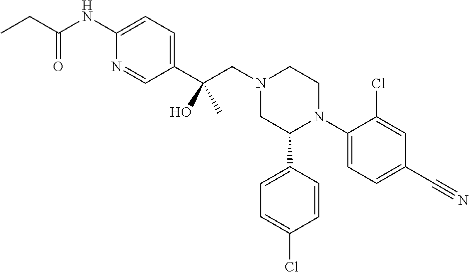 [SMILES: CCC(=O)Nc1ccc([C@](C)(O)CN2CCN(c3ccc(C#N)cc3Cl)[C@H](c3ccc(Cl)cc3)C2)cn1] |
| 1041 | 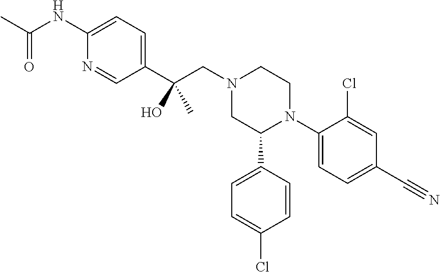 [SMILES: CC(=O)Nc1ccc([C@](C)(O)CN2CCN(c3ccc(C#N)cc3Cl)[C@H](c3ccc(Cl)cc3)C2)cn1] |
| 1042 | 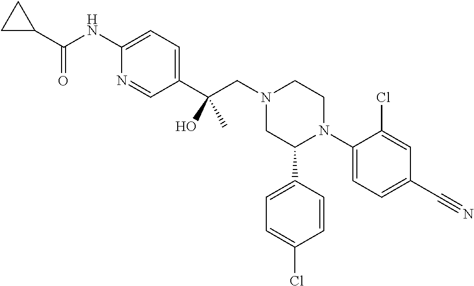 [SMILES: C[C@@](O)(CN1CCN(c2ccc(C#N)cc2Cl)[C@H](c2ccc(Cl)cc2)C1)c1ccc(NC(=O)C2CC2)nc1] |
| 1043 | 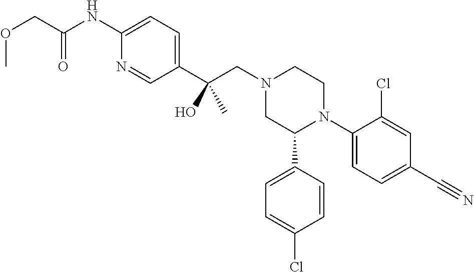 [SMILES: COCC(=O)Nc1ccc([C@](C)(O)CN2CCN(c3ccc(C#N)cc3Cl)[C@H](c3ccc(Cl)cc3)C2)cn1] |
| 1044 | 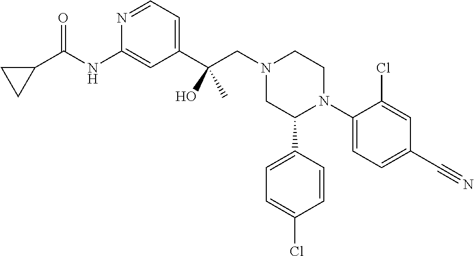 [SMILES: C[C@@](O)(CN1CCN(c2ccc(C#N)cc2Cl)[C@H](c2ccc(Cl)cc2)C1)c1ccnc(NC(=O)C2CC2)c1] |
| 1045 | 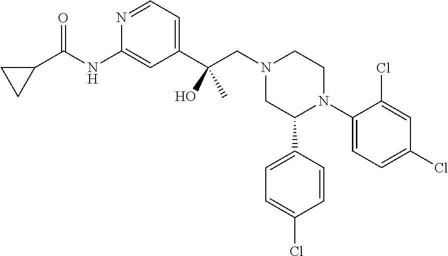 [SMILES: C[C@@](O)(CN1CCN(c2ccc(Cl)cc2Cl)[C@H](c2ccc(Cl)cc2)C1)c1ccnc(NC(=O)C2CC2)c1] |
| 1046 | 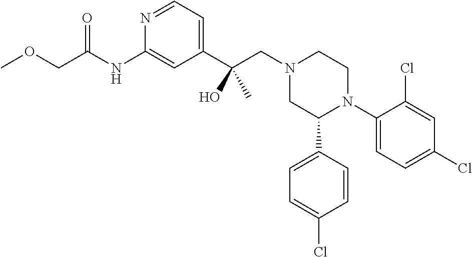 [SMILES: COCC(=O)Nc1cc([C@](C)(O)CN2CCN(c3ccc(Cl)cc3Cl)[C@H](c3ccc(Cl)cc3)C2)ccn1] |
| 1048 | 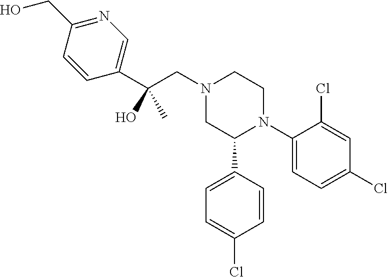 [SMILES: C[C@@](O)(CN1CCN(c2ccc(Cl)cc2Cl)[C@H](c2ccc(Cl)cc2)C1)c1ccc(CO)nc1] |
| 1049 | 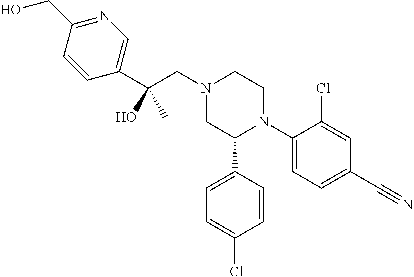 [SMILES: C[C@@](O)(CN1CCN(c2ccc(C#N)cc2Cl)[C@H](c2ccc(Cl)cc2)C1)c1ccc(CO)nc1] |
| 1050 | 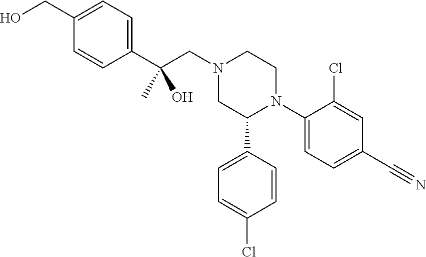 [SMILES: C[C@@](O)(CN1CCN(c2ccc(C#N)cc2Cl)[C@H](c2ccc(Cl)cc2)C1)c1ccc(CO)cc1] |
| 1051 | 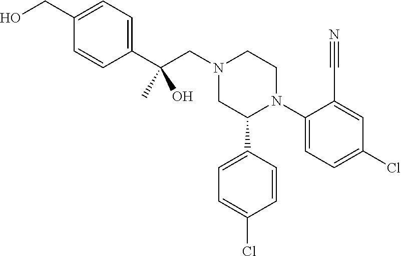 [SMILES: C[C@@](O)(CN1CCN(c2ccc(Cl)cc2C#N)[C@H](c2ccc(Cl)cc2)C1)c1ccc(CO)cc1] |
| 1052 | 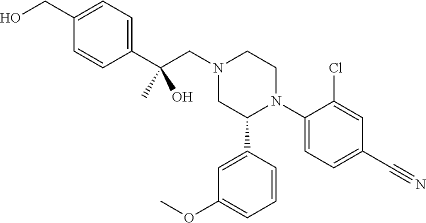 [SMILES: COc1cccc([C@@H]2CN(C[C@@](C)(O)c3ccc(CO)cc3)CCN2c2ccc(C#N)cc2Cl)c1] |
| 1053 | 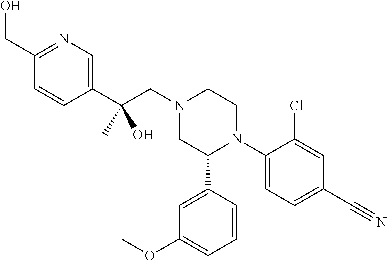 [SMILES: COc1cccc([C@@H]2CN(C[C@@](C)(O)c3ccc(CO)nc3)CCN2c2ccc(C#N)cc2Cl)c1] |
| 1054 | 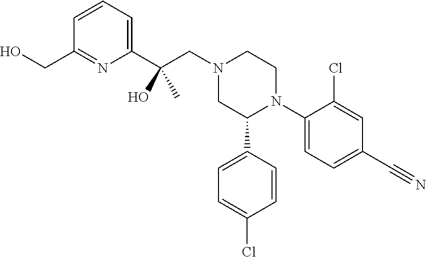 [SMILES: C[C@@](O)(CN1CCN(c2ccc(C#N)cc2Cl)[C@H](c2ccc(Cl)cc2)C1)c1cccc(CO)n1] |
| 1055 | 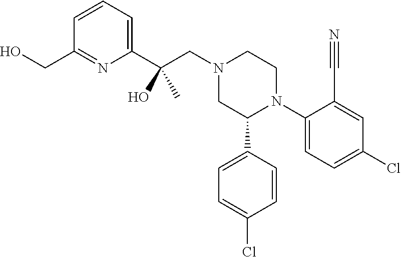 [SMILES: C[C@@](O)(CN1CCN(c2ccc(Cl)cc2C#N)[C@H](c2ccc(Cl)cc2)C1)c1cccc(CO)n1] |
| 1056 | 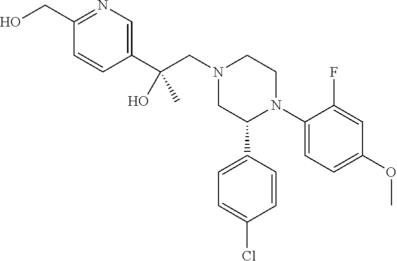 [SMILES: COc1ccc(N2CCN(C[C@@](C)(O)c3ccc(CO)nc3)C[C@H]2c2ccc(Cl)cc2)c(F)c1] |
| 1057 | 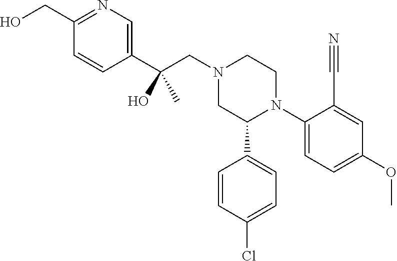 [SMILES: COc1ccc(N2CCN(C[C@@](C)(O)c3ccc(CO)nc3)C[C@H]2c2ccc(Cl)cc2)c(C#N)c1] |
| 1058 | 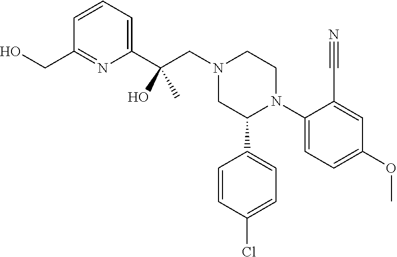 [SMILES: COc1ccc(N2CCN(C[C@@](C)(O)c3cccc(CO)n3)C[C@H]2c2ccc(Cl)cc2)c(C#N)c1] |
| 1059 | 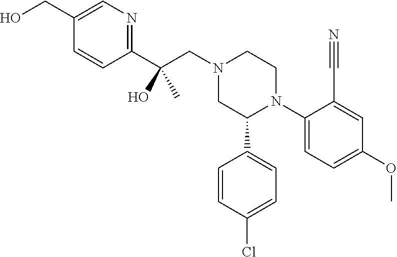 [SMILES: COc1ccc(N2CCN(C[C@@](C)(O)c3ccc(CO)cn3)C[C@H]2c2ccc(Cl)cc2)c(C#N)c1] |
| 1060 | 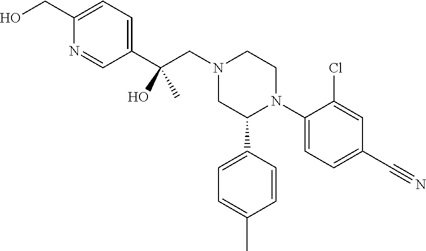 [SMILES: Cc1ccc([C@@H]2CN(C[C@@](C)(O)c3ccc(CO)nc3)CCN2c2ccc(C#N)cc2Cl)cc1] |
| 1061 | 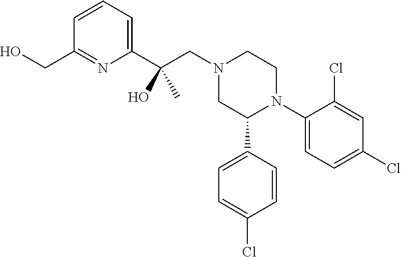 [SMILES: C[C@@](O)(CN1CCN(c2ccc(Cl)cc2Cl)[C@H](c2ccc(Cl)cc2)C1)c1cccc(CO)n1] |
| 1062a | 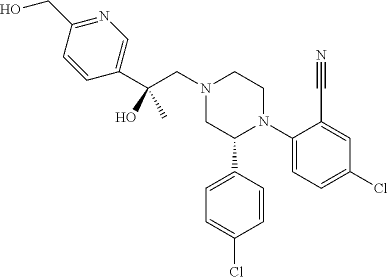 [SMILES: C[C@@](O)(CN1CCN(c2ccc(Cl)cc2C#N)[C@H](c2ccc(Cl)cc2)C1)c1ccc(CO)nc1] |
| 1062c | 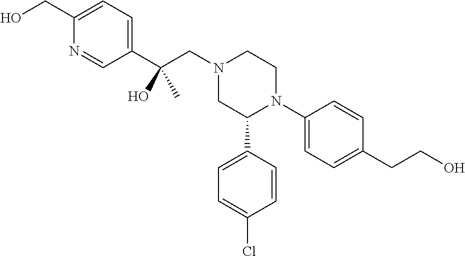 [SMILES: C[C@@](O)(CN1CCN(c2ccc(CCO)cc2)[C@H](c2ccc(Cl)cc2)C1)c1ccc(CO)nc1] |
| 1062b | 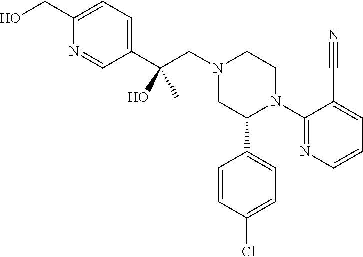 [SMILES: C[C@@](O)(CN1CCN(c2ncccc2C#N)[C@H](c2ccc(Cl)cc2)C1)c1ccc(CO)nc1] |
| 1063 | 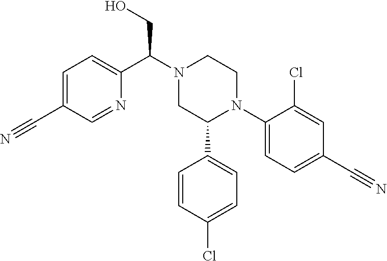 [SMILES: N#Cc1ccc([C@@H](CO)N2CCN(c3ccc(C#N)cc3Cl)[C@H](c3ccc(Cl)cc3)C2)nc1] |
| 1064 | 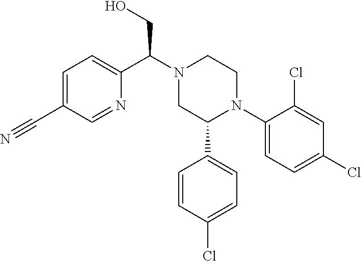 [SMILES: N#Cc1ccc([C@@H](CO)N2CCN(c3ccc(Cl)cc3Cl)[C@H](c3ccc(Cl)cc3)C2)nc1] |
| 1065 | 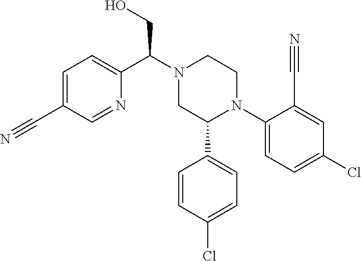 [SMILES: N#Cc1ccc([C@@H](CO)N2CCN(c3ccc(Cl)cc3C#N)[C@H](c3ccc(Cl)cc3)C2)nc1] |
| 1068 | 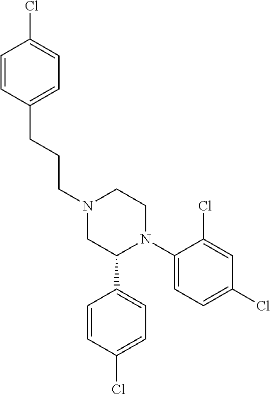 [SMILES: Clc1ccc(CCCN2CCN(c3ccc(Cl)cc3Cl)[C@H](c3ccc(Cl)cc3)C2)cc1] |
| 1069 | 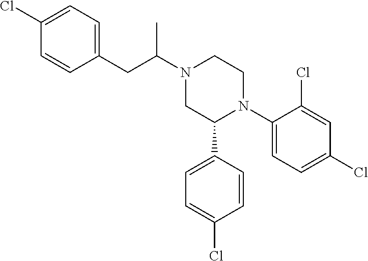 [SMILES: CC(Cc1ccc(Cl)cc1)N1CCN(c2ccc(Cl)cc2Cl)[C@H](c2ccc(Cl)cc2)C1] |
| 1070 | 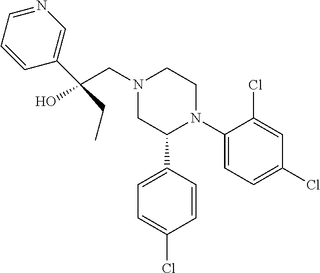 [SMILES: CC[C@](O)(CN1CCN(c2ccc(Cl)cc2Cl)[C@H](c2ccc(Cl)cc2)C1)c1cccnc1] |
| 1071 | 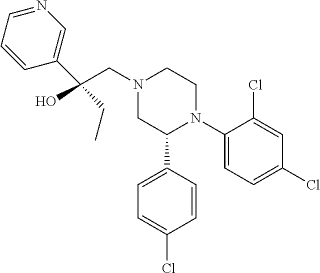 [SMILES: CC[C@@](O)(CN1CCN(c2ccc(Cl)cc2Cl)[C@H](c2ccc(Cl)cc2)C1)c1cccnc1] |
| 1072 | 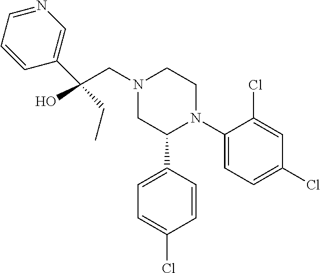 [SMILES: CC[C@@](O)(CN1CCN(c2ccc(Cl)cc2Cl)[C@H](c2ccc(Cl)cc2)C1)c1cccnc1] |
| 1073 | 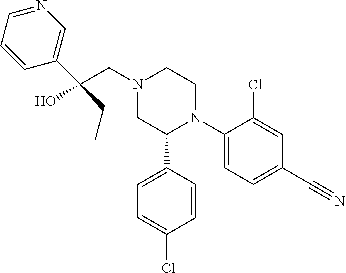 [SMILES: CC[C@](O)(CN1CCN(c2ccc(C#N)cc2Cl)[C@H](c2ccc(Cl)cc2)C1)c1cccnc1] |
| 1074 | 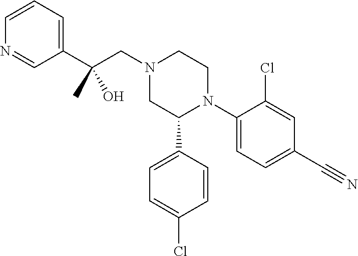 [SMILES: C[C@](O)(CN1CCN(c2ccc(C#N)cc2Cl)[C@H](c2ccc(Cl)cc2)C1)c1cccnc1] |
| 1075 | 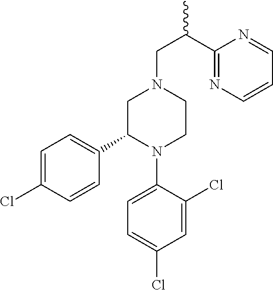 [SMILES: CC(CN1CCN(c2ccc(Cl)cc2Cl)[C@H](c2ccc(Cl)cc2)C1)c1ncccn1] |
| 1077 | 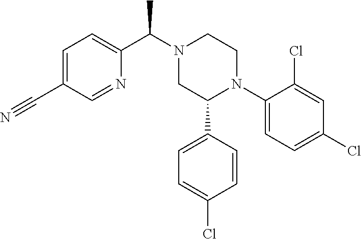 [SMILES: C[C@H](c1ccc(C#N)cn1)N1CCN(c2ccc(Cl)cc2Cl)[C@H](c2ccc(Cl)cc2)C1] |
| 1079 | 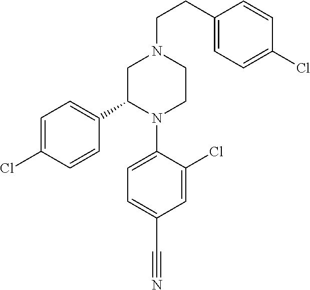 [SMILES: N#Cc1ccc(N2CCN(CCc3ccc(Cl)cc3)C[C@H]2c2ccc(Cl)cc2)c(Cl)c1] |
| 1080 | 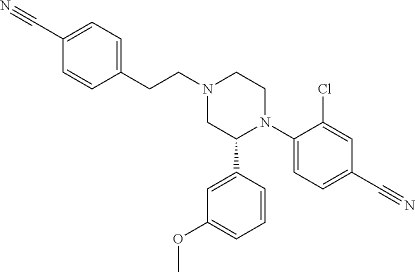 [SMILES: COc1cccc([C@@H]2CN(CCc3ccc(C#N)cc3)CCN2c2ccc(C#N)cc2Cl)c1] |
| 1081 | 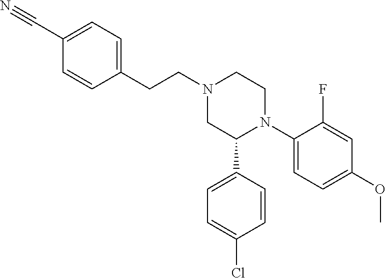 [SMILES: COc1ccc(N2CCN(CCc3ccc(C#N)cc3)C[C@H]2c2ccc(Cl)cc2)c(F)c1] |
| 1082 | 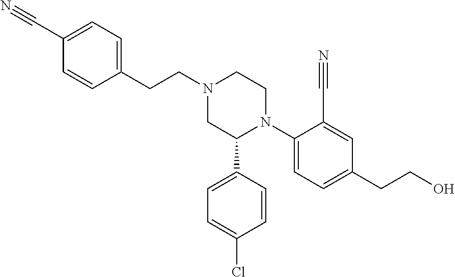 [SMILES: N#Cc1ccc(CCN2CCN(c3ccc(CCO)cc3C#N)[C@H](c3ccc(Cl)cc3)C2)cc1] |
| 1082b | 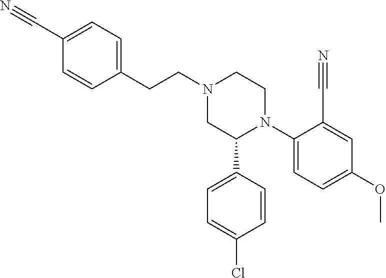 [SMILES: COc1ccc(N2CCN(CCc3ccc(C#N)cc3)C[C@H]2c2ccc(Cl)cc2)c(C#N)c1] |
| 1082c | 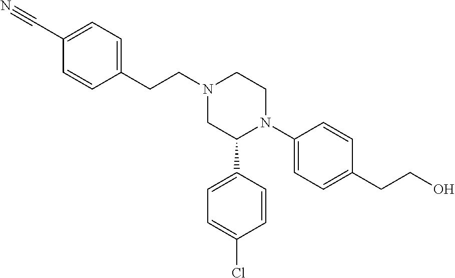 [SMILES: N#Cc1ccc(CCN2CCN(c3ccc(CCO)cc3)[C@H](c3ccc(Cl)cc3)C2)cc1] |
| 1083 | 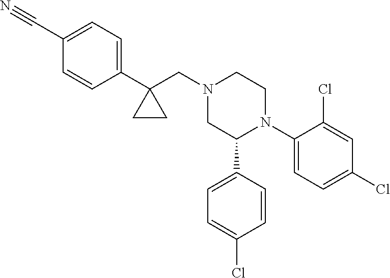 [SMILES: N#Cc1ccc(C2(CN3CCN(c4ccc(Cl)cc4Cl)[C@H](c4ccc(Cl)cc4)C3)CC2)cc1] |
| 1084 | 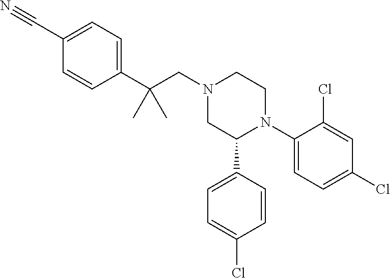 [SMILES: CC(C)(CN1CCN(c2ccc(Cl)cc2Cl)[C@H](c2ccc(Cl)cc2)C1)c1ccc(C#N)cc1] |
| 1085 | 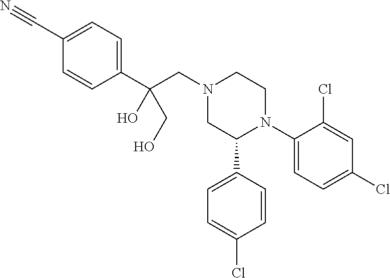 [SMILES: N#Cc1ccc(C(O)(CO)CN2CCN(c3ccc(Cl)cc3Cl)[C@H](c3ccc(Cl)cc3)C2)cc1] |
| 1086 | 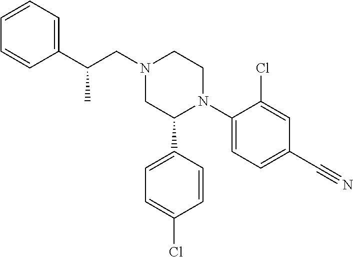 [SMILES: C[C@@H](CN1CCN(c2ccc(C#N)cc2Cl)[C@H](c2ccc(Cl)cc2)C1)c1ccccc1] |
| 1087a | 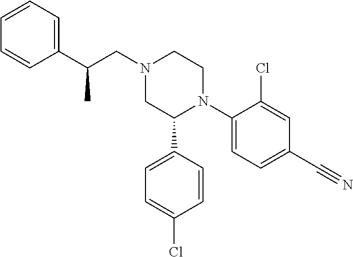 [SMILES: C[C@H](CN1CCN(c2ccc(C#N)cc2Cl)[C@H](c2ccc(Cl)cc2)C1)c1ccccc1] |
| 1088 | 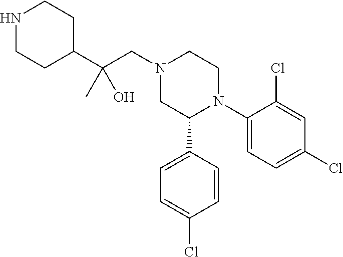 [SMILES: CC(O)(CN1CCN(c2ccc(Cl)cc2Cl)[C@H](c2ccc(Cl)cc2)C1)C1CCNCC1] |
| 1089 | 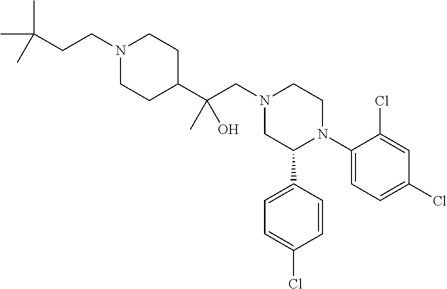 [SMILES: CC(C)(C)CCN1CCC(C(C)(O)CN2CCN(c3ccc(Cl)cc3Cl)[C@H](c3ccc(Cl)cc3)C2)CC1] |
| 1090 | 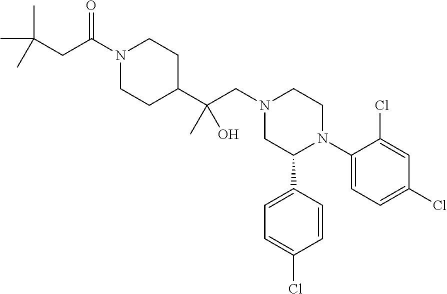 [SMILES: CC(C)(C)CC(=O)N1CCC(C(C)(O)CN2CCN(c3ccc(Cl)cc3Cl)[C@H](c3ccc(Cl)cc3)C2)CC1] |
| 1091 | 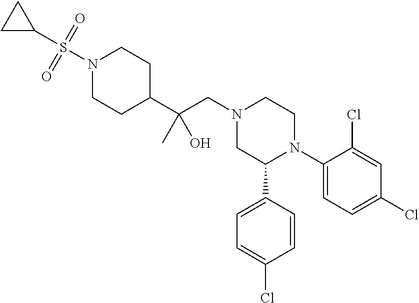 [SMILES: CC(O)(CN1CCN(c2ccc(Cl)cc2Cl)[C@H](c2ccc(Cl)cc2)C1)C1CCN(S(=O)(=O)C2CC2)CC1] |
| 1092 | 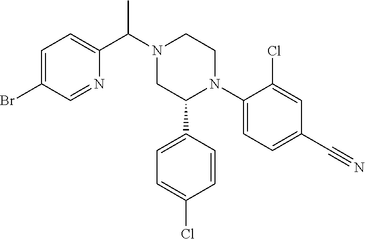 [SMILES: CC(c1ccc(Br)cn1)N1CCN(c2ccc(C#N)cc2Cl)[C@H](c2ccc(Cl)cc2)C1] |
| 1093 | 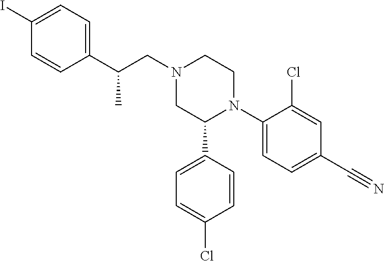 [SMILES: C[C@@H](CN1CCN(c2ccc(C#N)cc2Cl)[C@H](c2ccc(Cl)cc2)C1)c1ccc(I)cc1] |
| 1094 | 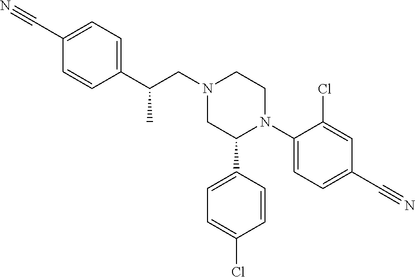 [SMILES: C[C@@H](CN1CCN(c2ccc(C#N)cc2Cl)[C@H](c2ccc(Cl)cc2)C1)c1ccc(C#N)cc1] |
| 1095 | 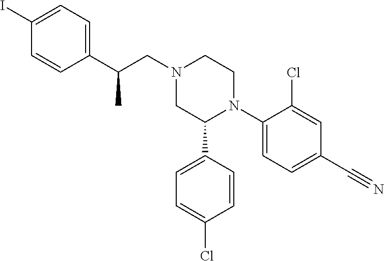 [SMILES: C[C@H](CN1CCN(c2ccc(C#N)cc2Cl)[C@H](c2ccc(Cl)cc2)C1)c1ccc(I)cc1] |
| 1096 | 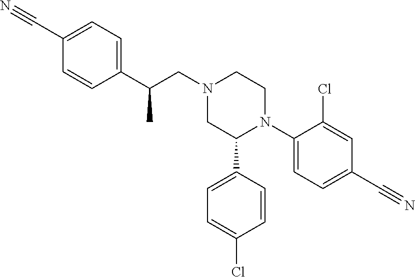 [SMILES: C[C@H](CN1CCN(c2ccc(C#N)cc2Cl)[C@H](c2ccc(Cl)cc2)C1)c1ccc(C#N)cc1] |
| 1097 | 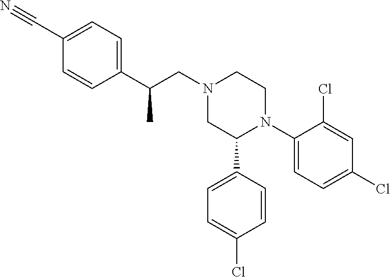 [SMILES: C[C@H](CN1CCN(c2ccc(Cl)cc2Cl)[C@H](c2ccc(Cl)cc2)C1)c1ccc(C#N)cc1] |
| 1098 | 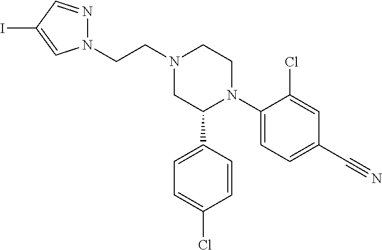 [SMILES: N#Cc1ccc(N2CCN(CCn3cc(I)cn3)C[C@H]2c2ccc(Cl)cc2)c(Cl)c1] |
| 1099 | 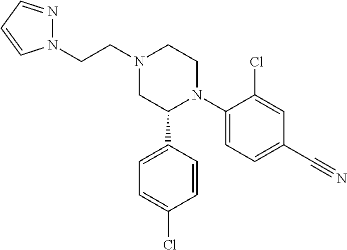 [SMILES: N#Cc1ccc(N2CCN(CCn3cccn3)C[C@H]2c2ccc(Cl)cc2)c(Cl)c1] |
| 1100 | 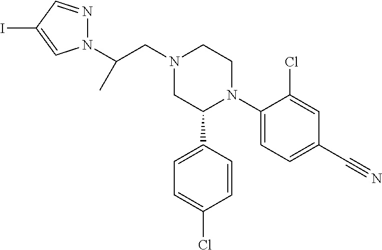 [SMILES: CC(CN1CCN(c2ccc(C#N)cc2Cl)[C@H](c2ccc(Cl)cc2)C1)n1cc(I)cn1] |
| 1101 | 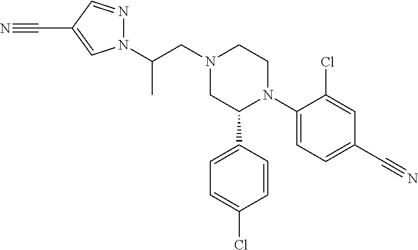 [SMILES: CC(CN1CCN(c2ccc(C#N)cc2Cl)[C@H](c2ccc(Cl)cc2)C1)n1cc(C#N)cn1] |
| 1102 | 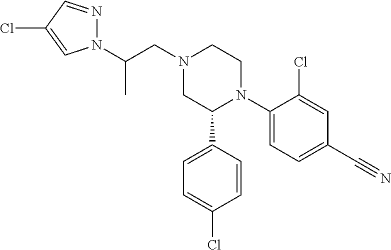 [SMILES: CC(CN1CCN(c2ccc(C#N)cc2Cl)[C@H](c2ccc(Cl)cc2)C1)n1cc(Cl)cn1] |
| 1103 | 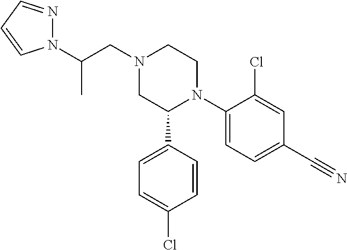 [SMILES: CC(CN1CCN(c2ccc(C#N)cc2Cl)[C@H](c2ccc(Cl)cc2)C1)n1cccn1] |
| 1104a | 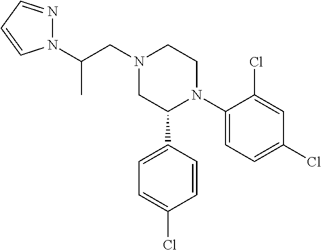 [SMILES: CC(CN1CCN(c2ccc(Cl)cc2Cl)[C@H](c2ccc(Cl)cc2)C1)n1cccn1] |
| 1104b | 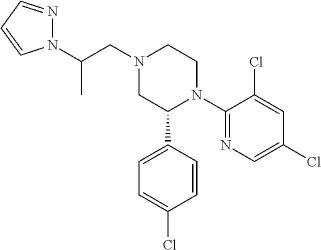 [SMILES: CC(CN1CCN(c2ncc(Cl)cc2Cl)[C@H](c2ccc(Cl)cc2)C1)n1cccn1] |
| 1105 | 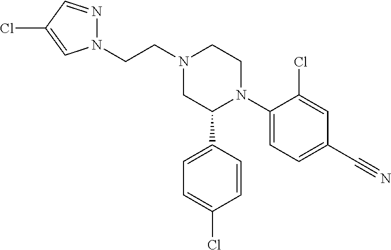 [SMILES: N#Cc1ccc(N2CCN(CCn3cc(Cl)cn3)C[C@H]2c2ccc(Cl)cc2)c(Cl)c1] |
| 1106 | 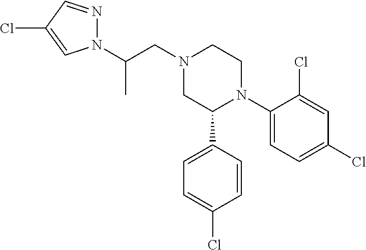 [SMILES: CC(CN1CCN(c2ccc(Cl)cc2Cl)[C@H](c2ccc(Cl)cc2)C1)n1cc(Cl)cn1] |
| 1107 | 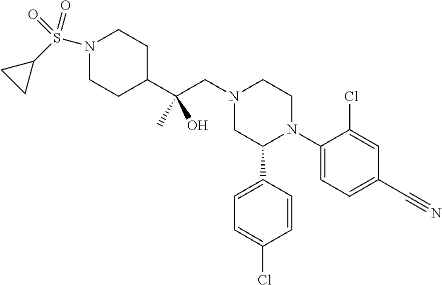 [SMILES: C[C@@](O)(CN1CCN(c2ccc(C#N)cc2Cl)[C@H](c2ccc(Cl)cc2)C1)C1CCN(S(=O)(=O)C2CC2)CC1] |
| 1108 | 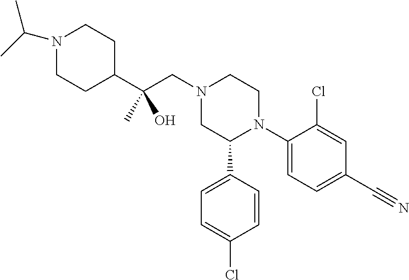 [SMILES: CC(C)N1CCC([C@](C)(O)CN2CCN(c3ccc(C#N)cc3Cl)[C@H](c3ccc(Cl)cc3)C2)CC1] |
| 1109 | 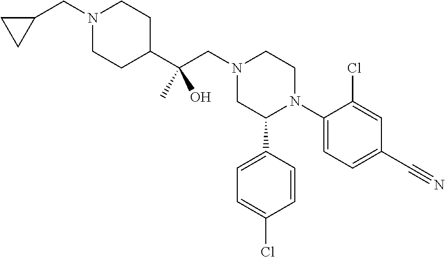 [SMILES: C[C@@](O)(CN1CCN(c2ccc(C#N)cc2Cl)[C@H](c2ccc(Cl)cc2)C1)C1CCN(CC2CC2)CC1] |
| 1110 | 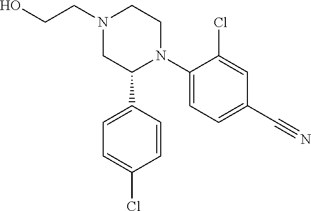 [SMILES: N#Cc1ccc(N2CCN(CCO)C[C@H]2c2ccc(Cl)cc2)c(Cl)c1] |
| 1111 | 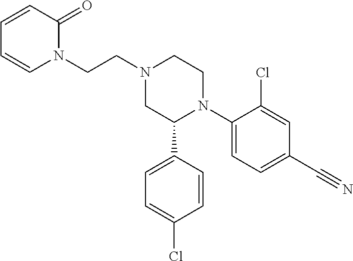 [SMILES: N#Cc1ccc(N2CCN(CCn3ccccc3=O)C[C@H]2c2ccc(Cl)cc2)c(Cl)c1] |
| 1112 | 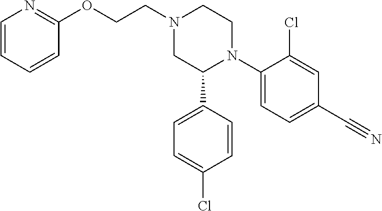 [SMILES: N#Cc1ccc(N2CCN(CCOc3ccccn3)C[C@H]2c2ccc(Cl)cc2)c(Cl)c1] |
| 1113 | 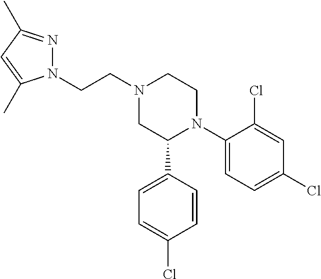 [SMILES: Cc1cc(C)n(CCN2CCN(c3ccc(Cl)cc3Cl)[C@H](c3ccc(Cl)cc3)C2)n1] |
| 1114 | 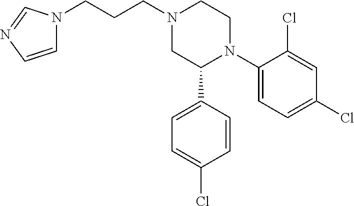 [SMILES: Clc1ccc([C@@H]2CN(CCCn3ccnc3)CCN2c2ccc(Cl)cc2Cl)cc1] |
| 1115 | 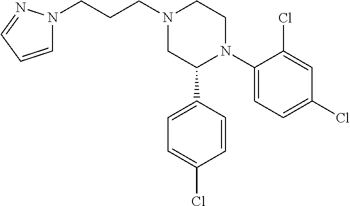 [SMILES: Clc1ccc([C@@H]2CN(CCCn3cccn3)CCN2c2ccc(Cl)cc2Cl)cc1] |
| 1116 | 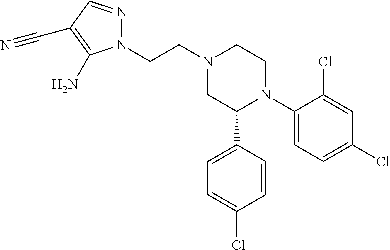 [SMILES: N#Cc1cnn(CCN2CCN(c3ccc(Cl)cc3Cl)[C@H](c3ccc(Cl)cc3)C2)c1N] |
| 1117 | 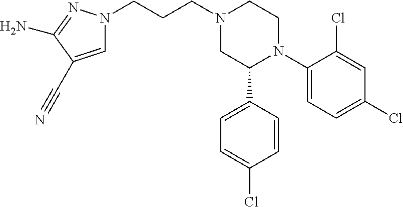 [SMILES: N#Cc1cn(CCCN2CCN(c3ccc(Cl)cc3Cl)[C@H](c3ccc(Cl)cc3)C2)nc1N] |
| 1118 | 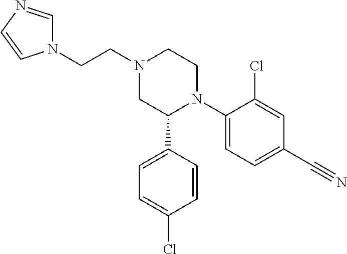 [SMILES: N#Cc1ccc(N2CCN(CCn3ccnc3)C[C@H]2c2ccc(Cl)cc2)c(Cl)c1] |
| 1119 | 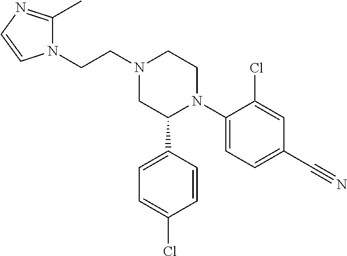 [SMILES: Cc1nccn1CCN1CCN(c2ccc(C#N)cc2Cl)[C@H](c2ccc(Cl)cc2)C1] |
| 1120 | 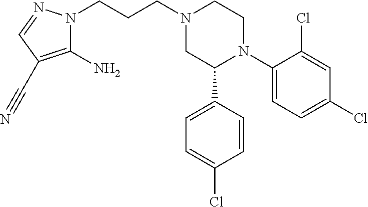 [SMILES: N#Cc1cnn(CCCN2CCN(c3ccc(Cl)cc3Cl)[C@H](c3ccc(Cl)cc3)C2)c1N] |
| 1121 | 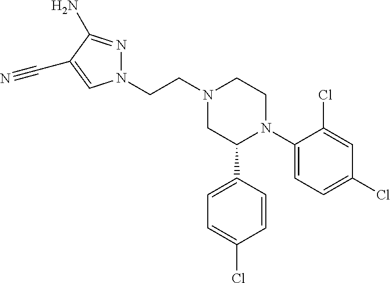 [SMILES: N#Cc1cn(CCN2CCN(c3ccc(Cl)cc3Cl)[C@H](c3ccc(Cl)cc3)C2)nc1N] |
| 1122 | 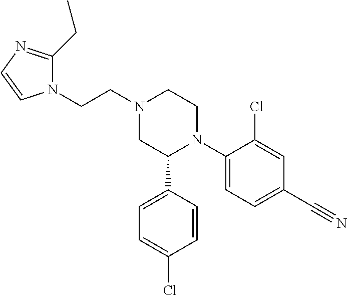 [SMILES: CCc1nccn1CCN1CCN(c2ccc(C#N)cc2Cl)[C@H](c2ccc(Cl)cc2)C1] |
| 1123 | 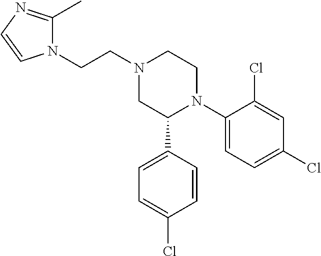 [SMILES: Cc1nccn1CCN1CCN(c2ccc(Cl)cc2Cl)[C@H](c2ccc(Cl)cc2)C1] |
| 1124 | 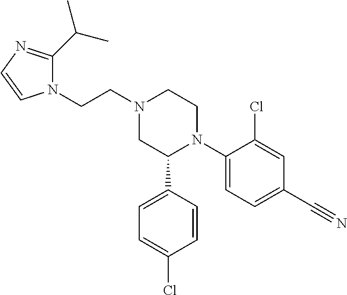 [SMILES: CC(C)c1nccn1CCN1CCN(c2ccc(C#N)cc2Cl)[C@H](c2ccc(Cl)cc2)C1] |
| 1125 | 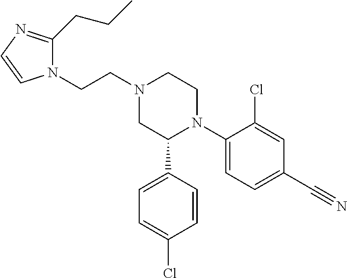 [SMILES: CCCc1nccn1CCN1CCN(c2ccc(C#N)cc2Cl)[C@H](c2ccc(Cl)cc2)C1] |
| 1126 | 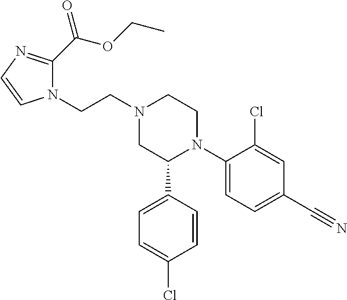 [SMILES: CCOC(=O)c1nccn1CCN1CCN(c2ccc(C#N)cc2Cl)[C@H](c2ccc(Cl)cc2)C1] |
| 1127 | 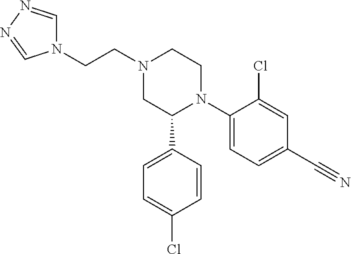 [SMILES: N#Cc1ccc(N2CCN(CCn3cnnc3)C[C@H]2c2ccc(Cl)cc2)c(Cl)c1] |
| 1128 | 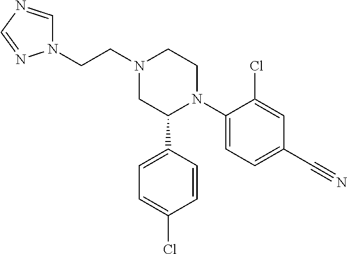 [SMILES: N#Cc1ccc(N2CCN(CCn3cncn3)C[C@H]2c2ccc(Cl)cc2)c(Cl)c1] |
| 1129 | 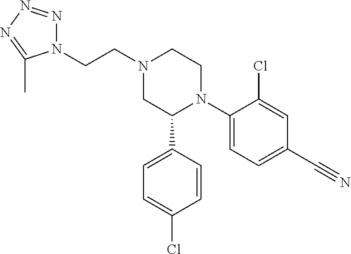 [SMILES: Cc1nnnn1CCN1CCN(c2ccc(C#N)cc2Cl)[C@H](c2ccc(Cl)cc2)C1] |
| 1130 | 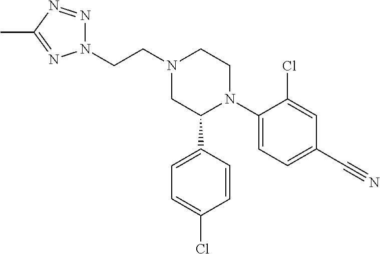 [SMILES: Cc1nnn(CCN2CCN(c3ccc(C#N)cc3Cl)[C@H](c3ccc(Cl)cc3)C2)n1] |
| 1131 | 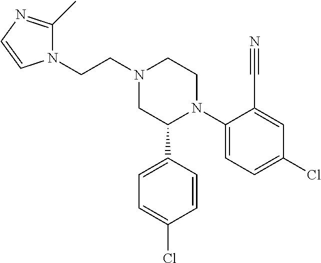 [SMILES: Cc1nccn1CCN1CCN(c2ccc(Cl)cc2C#N)[C@H](c2ccc(Cl)cc2)C1] |
| 1131 | 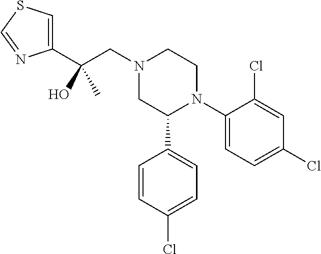 [SMILES: C[C@@](O)(CN1CCN(c2ccc(Cl)cc2Cl)[C@H](c2ccc(Cl)cc2)C1)c1cscn1] |
| 1132 | 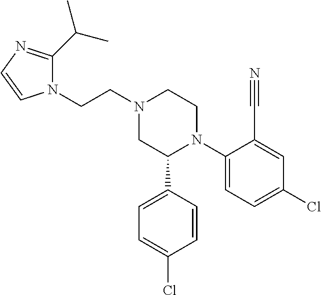 [SMILES: CC(C)c1nccn1CCN1CCN(c2ccc(Cl)cc2C#N)[C@H](c2ccc(Cl)cc2)C1] |
| 1133 | 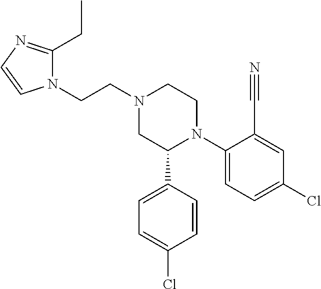 [SMILES: CCc1nccn1CCN1CCN(c2ccc(Cl)cc2C#N)[C@H](c2ccc(Cl)cc2)C1] |
| 1134 | 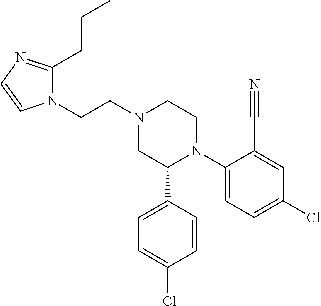 [SMILES: CCCc1nccn1CCN1CCN(c2ccc(Cl)cc2C#N)[C@H](c2ccc(Cl)cc2)C1] |
| 1135 | 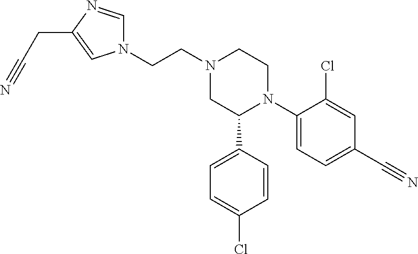 [SMILES: N#CCc1cn(CCN2CCN(c3ccc(C#N)cc3Cl)[C@H](c3ccc(Cl)cc3)C2)cn1] |
| 1136 | 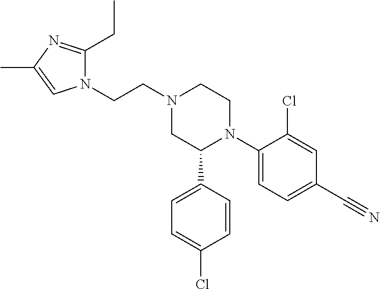 [SMILES: CCc1nc(C)cn1CCN1CCN(c2ccc(C#N)cc2Cl)[C@H](c2ccc(Cl)cc2)C1] |
| 1137 | 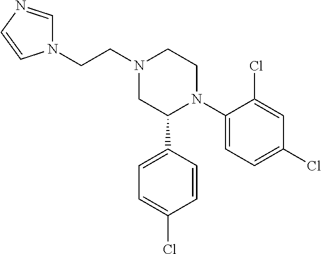 [SMILES: Clc1ccc([C@@H]2CN(CCn3ccnc3)CCN2c2ccc(Cl)cc2Cl)cc1] |
| 1138 | 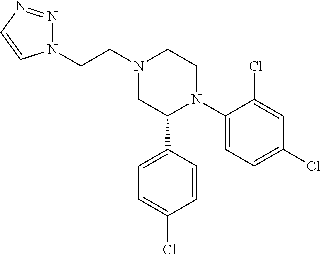 [SMILES: Clc1ccc([C@@H]2CN(CCn3ccnn3)CCN2c2ccc(Cl)cc2Cl)cc1] |
| 1139 | 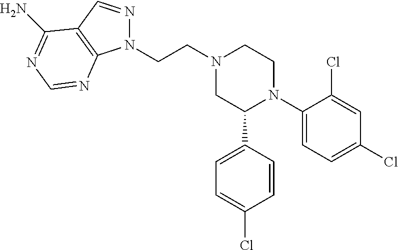 [SMILES: Nc1ncnc2c1cnn2CCN1CCN(c2ccc(Cl)cc2Cl)[C@H](c2ccc(Cl)cc2)C1] |
| 1140 | 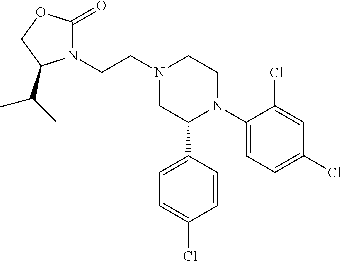 [SMILES: CC(C)[C@H]1COC(=O)N1CCN1CCN(c2ccc(Cl)cc2Cl)[C@H](c2ccc(Cl)cc2)C1] |
| 1141 | 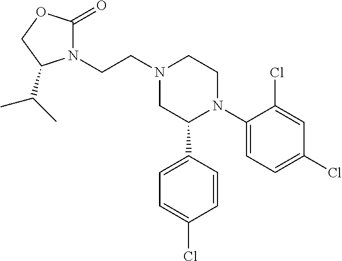 [SMILES: CC(C)[C@@H]1COC(=O)N1CCN1CCN(c2ccc(Cl)cc2Cl)[C@H](c2ccc(Cl)cc2)C1] |
| 1142 | 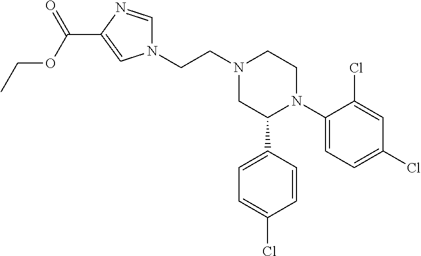 [SMILES: CCOC(=O)c1cn(CCN2CCN(c3ccc(Cl)cc3Cl)[C@H](c3ccc(Cl)cc3)C2)cn1] |
| 1143 | 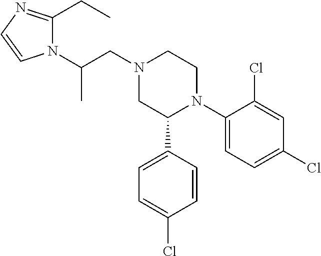 [SMILES: CCc1nccn1C(C)CN1CCN(c2ccc(Cl)cc2Cl)[C@H](c2ccc(Cl)cc2)C1] |
| 1144 | 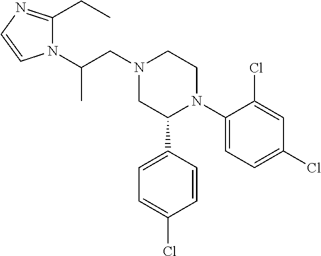 [SMILES: CCc1nccn1C(C)CN1CCN(c2ccc(Cl)cc2Cl)[C@H](c2ccc(Cl)cc2)C1] |
| 1145 | 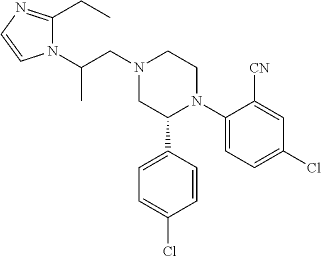 [SMILES: CCc1nccn1C(C)CN1CCN(c2ccc(Cl)cc2C#N)[C@H](c2ccc(Cl)cc2)C1] |
| 1146 | 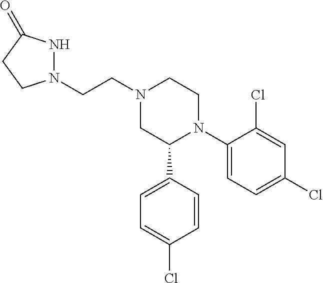 [SMILES: O=C1CCN(CCN2CCN(c3ccc(Cl)cc3Cl)[C@H](c3ccc(Cl)cc3)C2)N1] |
| 1147 | 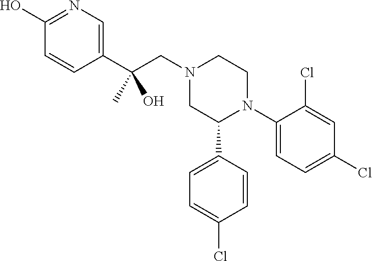 [SMILES: C[C@@](O)(CN1CCN(c2ccc(Cl)cc2Cl)[C@H](c2ccc(Cl)cc2)C1)c1ccc(O)nc1] |
| 1148 | 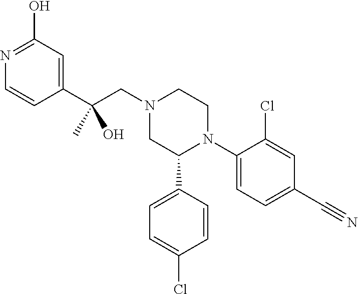 [SMILES: C[C@@](O)(CN1CCN(c2ccc(C#N)cc2Cl)[C@H](c2ccc(Cl)cc2)C1)c1ccnc(O)c1] |
| 1149 | 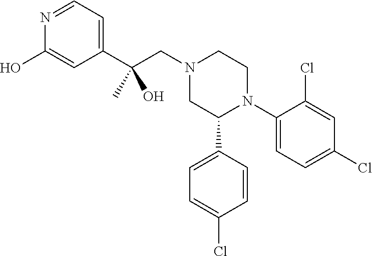 [SMILES: C[C@@](O)(CN1CCN(c2ccc(Cl)cc2Cl)[C@H](c2ccc(Cl)cc2)C1)c1ccnc(O)c1] |
| 1150 | 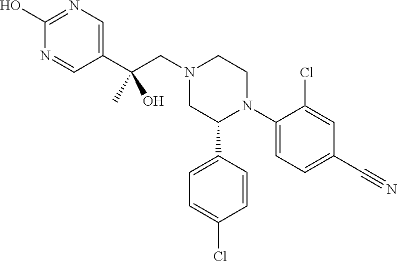 [SMILES: C[C@@](O)(CN1CCN(c2ccc(C#N)cc2Cl)[C@H](c2ccc(Cl)cc2)C1)c1cnc(O)nc1] |
| 1151 | 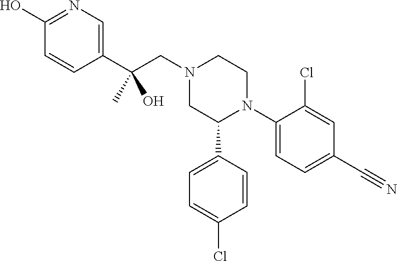 [SMILES: C[C@@](O)(CN1CCN(c2ccc(C#N)cc2Cl)[C@H](c2ccc(Cl)cc2)C1)c1ccc(O)nc1] |
| 1152 | 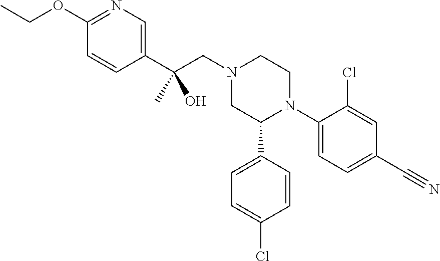 [SMILES: CCOc1ccc([C@](C)(O)CN2CCN(c3ccc(C#N)cc3Cl)[C@H](c3ccc(Cl)cc3)C2)cn1] |
| 1153 | 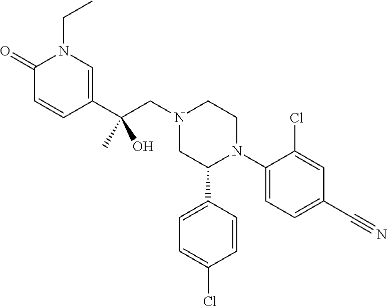 [SMILES: CCn1cc([C@](C)(O)CN2CCN(c3ccc(C#N)cc3Cl)[C@H](c3ccc(Cl)cc3)C2)ccc1=O] |
| 1154 | 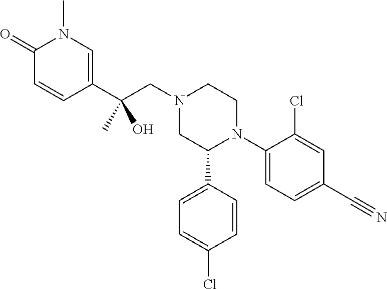 [SMILES: Cn1cc([C@](C)(O)CN2CCN(c3ccc(C#N)cc3Cl)[C@H](c3ccc(Cl)cc3)C2)ccc1=O] |
| 1155b | 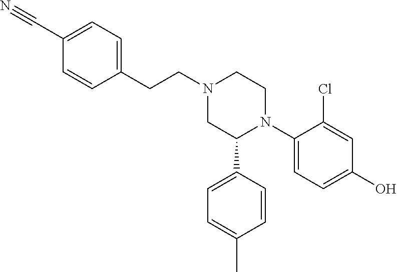 [SMILES: Cc1ccc([C@@H]2CN(CCc3ccc(C#N)cc3)CCN2c2ccc(O)cc2Cl)cc1] |
| 1156 | 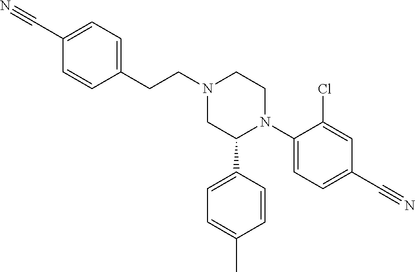 [SMILES: Cc1ccc([C@@H]2CN(CCc3ccc(C#N)cc3)CCN2c2ccc(C#N)cc2Cl)cc1] |
| 1158 | 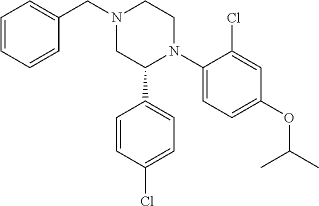 [SMILES: CC(C)Oc1ccc(N2CCN(Cc3ccccc3)C[C@H]2c2ccc(Cl)cc2)c(Cl)c1] |
| 1159 | 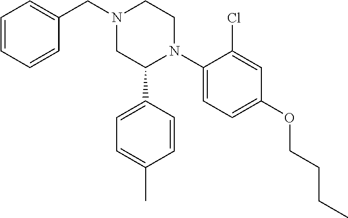 [SMILES: CCCCOc1ccc(N2CCN(Cc3ccccc3)C[C@H]2c2ccc(C)cc2)c(Cl)c1] |
| 1160 | 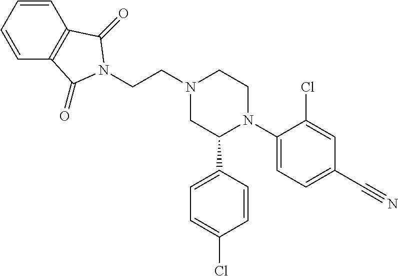 [SMILES: N#Cc1ccc(N2CCN(CCN3C(=O)c4ccccc4C3=O)C[C@H]2c2ccc(Cl)cc2)c(Cl)c1] |
| 1161 | 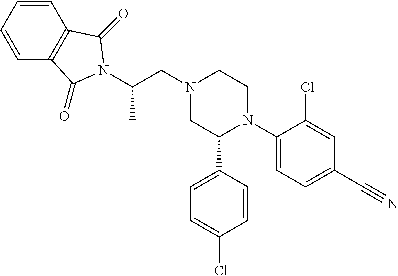 [SMILES: C[C@@H](CN1CCN(c2ccc(C#N)cc2Cl)[C@H](c2ccc(Cl)cc2)C1)N1C(=O)c2ccccc2C1=O] |
| 1162 | 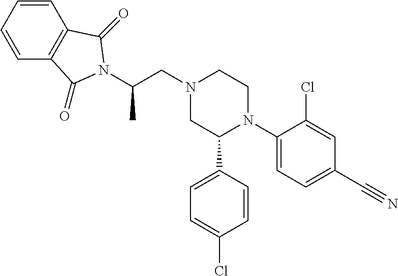 [SMILES: C[C@H](CN1CCN(c2ccc(C#N)cc2Cl)[C@H](c2ccc(Cl)cc2)C1)N1C(=O)c2ccccc2C1=O] |
| 1163 | 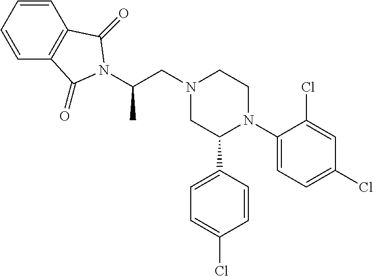 [SMILES: C[C@H](CN1CCN(c2ccc(Cl)cc2Cl)[C@H](c2ccc(Cl)cc2)C1)N1C(=O)c2ccccc2C1=O] |
| 1164 | 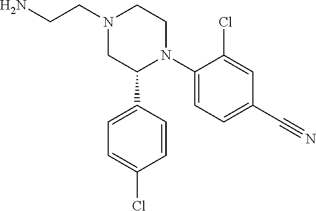 [SMILES: N#Cc1ccc(N2CCN(CCN)C[C@H]2c2ccc(Cl)cc2)c(Cl)c1] |
| 1165 | 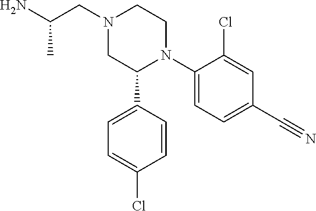 [SMILES: C[C@H](N)CN1CCN(c2ccc(C#N)cc2Cl)[C@H](c2ccc(Cl)cc2)C1] |
| 1167a | 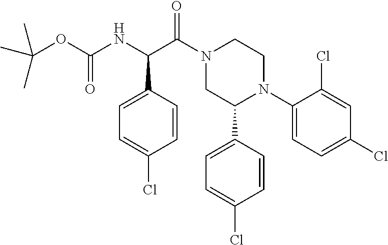 [SMILES: CC(C)(C)OC(=O)N[C@@H](C(=O)N1CCN(c2ccc(Cl)cc2Cl)[C@H](c2ccc(Cl)cc2)C1)c1ccc(Cl)cc1] |
| 1167b | 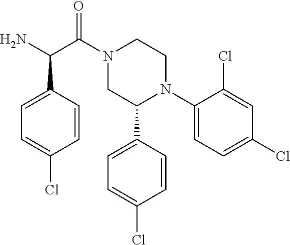 [SMILES: N[C@@H](C(=O)N1CCN(c2ccc(Cl)cc2Cl)[C@H](c2ccc(Cl)cc2)C1)c1ccc(Cl)cc1] |
| 1168 | 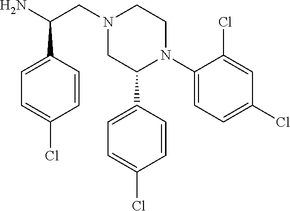 [SMILES: N[C@@H](CN1CCN(c2ccc(Cl)cc2Cl)[C@H](c2ccc(Cl)cc2)C1)c1ccc(Cl)cc1] |
| 1169 | 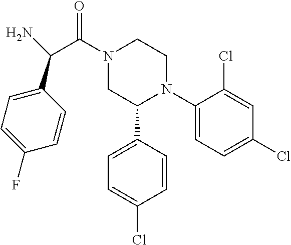 [SMILES: N[C@@H](C(=O)N1CCN(c2ccc(Cl)cc2Cl)[C@H](c2ccc(Cl)cc2)C1)c1ccc(F)cc1] |
| 1170 | 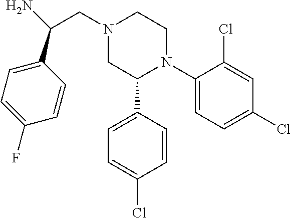 [SMILES: N[C@@H](CN1CCN(c2ccc(Cl)cc2Cl)[C@H](c2ccc(Cl)cc2)C1)c1ccc(F)cc1] |
| 1171 | 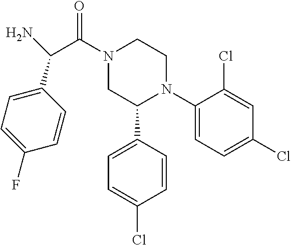 [SMILES: N[C@H](C(=O)N1CCN(c2ccc(Cl)cc2Cl)[C@H](c2ccc(Cl)cc2)C1)c1ccc(F)cc1] |
| 1172 | 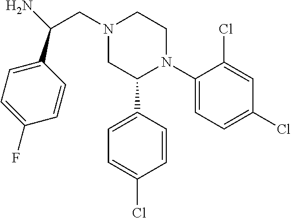 [SMILES: N[C@@H](CN1CCN(c2ccc(Cl)cc2Cl)[C@H](c2ccc(Cl)cc2)C1)c1ccc(F)cc1] |
| 1173 | 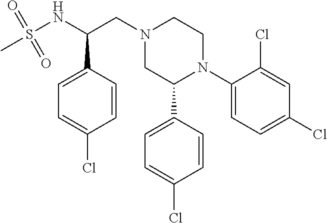 [SMILES: CS(=O)(=O)N[C@@H](CN1CCN(c2ccc(Cl)cc2Cl)[C@H](c2ccc(Cl)cc2)C1)c1ccc(Cl)cc1] |
| 1174 | 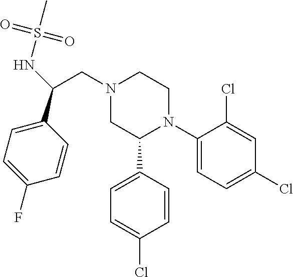 [SMILES: CS(=O)(=O)N[C@@H](CN1CCN(c2ccc(Cl)cc2Cl)[C@H](c2ccc(Cl)cc2)C1)c1ccc(F)cc1] |
| 1175 | 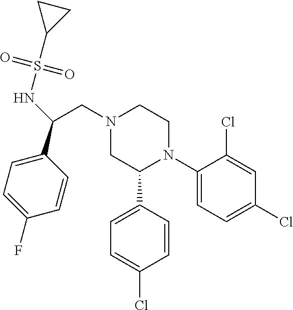 [SMILES: O=S(=O)(N[C@@H](CN1CCN(c2ccc(Cl)cc2Cl)[C@H](c2ccc(Cl)cc2)C1)c1ccc(F)cc1)C1CC1] |
| 1176 | 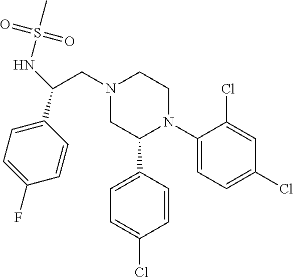 [SMILES: CS(=O)(=O)N[C@H](CN1CCN(c2ccc(Cl)cc2Cl)[C@H](c2ccc(Cl)cc2)C1)c1ccc(F)cc1] |
| 1177 | 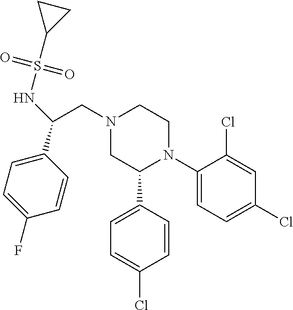 [SMILES: O=S(=O)(N[C@H](CN1CCN(c2ccc(Cl)cc2Cl)[C@H](c2ccc(Cl)cc2)C1)c1ccc(F)cc1)C1CC1] |
| 1178 | 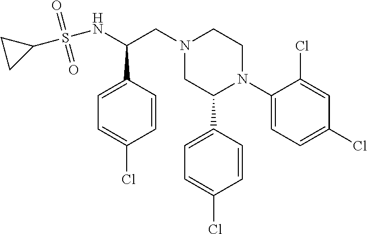 [SMILES: O=S(=O)(N[C@@H](CN1CCN(c2ccc(Cl)cc2Cl)[C@H](c2ccc(Cl)cc2)C1)c1ccc(Cl)cc1)C1CC1] |
| 1179 | 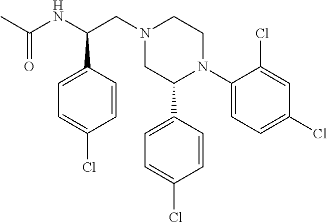 [SMILES: CC(=O)N[C@@H](CN1CCN(c2ccc(Cl)cc2Cl)[C@H](c2ccc(Cl)cc2)C1)c1ccc(Cl)cc1] |
| 1180 | 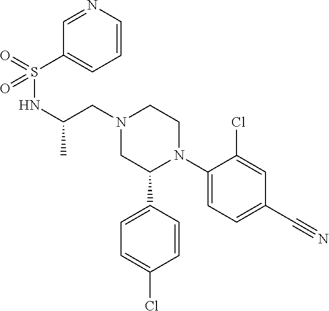 [SMILES: C[C@@H](CN1CCN(c2ccc(C#N)cc2Cl)[C@H](c2ccc(Cl)cc2)C1)NS(=O)(=O)c1cccnc1] |
| 1181 | 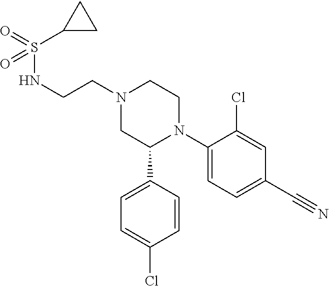 [SMILES: N#Cc1ccc(N2CCN(CCNS(=O)(=O)C3CC3)C[C@H]2c2ccc(Cl)cc2)c(Cl)c1] |
| 1182 | 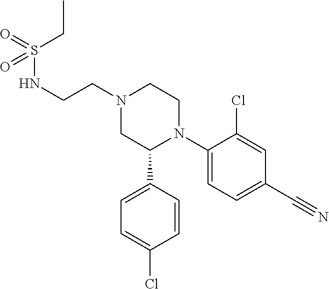 [SMILES: CCS(=O)(=O)NCCN1CCN(c2ccc(C#N)cc2Cl)[C@H](c2ccc(Cl)cc2)C1] |
| 1183 | 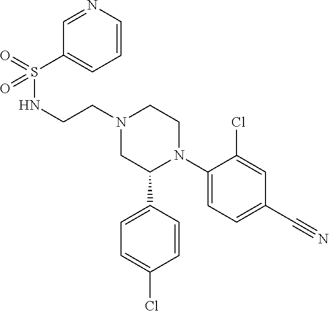 [SMILES: N#Cc1ccc(N2CCN(CCNS(=O)(=O)c3cccnc3)C[C@H]2c2ccc(Cl)cc2)c(Cl)c1] |
| 1184 | 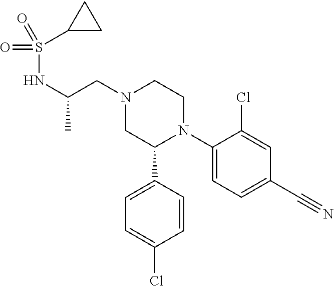 [SMILES: C[C@@H](CN1CCN(c2ccc(C#N)cc2Cl)[C@H](c2ccc(Cl)cc2)C1)NS(=O)(=O)C1CC1] |
| 1185 | 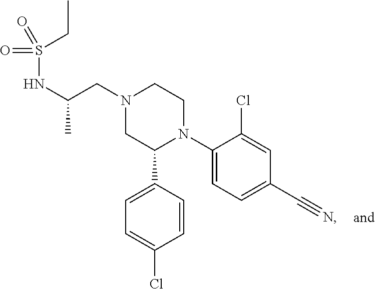 [SMILES: CCS(=O)(=O)N[C@@H](C)CN1CCN(c2ccc(C#N)cc2Cl)[C@H](c2ccc(Cl)cc2)C1] |
| 1186 | 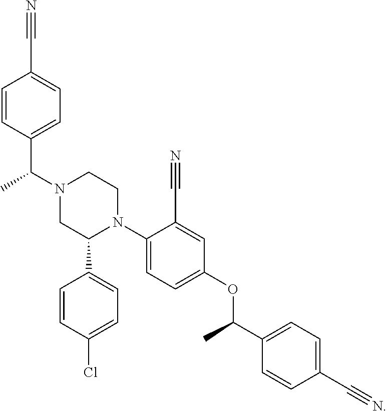 [SMILES: C[C@H](c1ccc(C#N)cc1)N1CCN(c2ccc(O[C@H](C)c3ccc(C#N)cc3)cc2C#N)[C@H](c2ccc(Cl)cc2)C1] |

2. A pharmaceutical composition comprising:
at least one compound according to claim 1, or a pharmaceutically acceptable salt thereof.

3. A composition comprising: at least one compound of claim 1, or a pharmaceutically acceptable salt thereof; and at least one additional active agent other than a compound of claim 1.

4. A composition of claim 3, wherein said at least one additional active agent is selected from a centrally acting agent and a peripheral acting agent.

5. A composition of claim 3, wherein said at least one additional active agent is selected from a histamine-3 receptor antagonist and a NPY5 antagonist.

6. A composition of claim 3, wherein said at least one additional active agent is selected from a microsomal triglyceride transfer protein (MTP) inhibitor.

7. A composition comprising: at least one compound of claim 1, or a pharmaceutically acceptable salt thereof; and at least one cholesterol lowering compound.

8. The composition of claim 7, wherein said at least one cholesterol lowering compound is at least one sterol absorption inhibitor or at least one 5α-stanol absorption inhibitor.

9. The composition of claim 7, wherein said at least one cholesterol lowering compound is at least one substituted azetidinone compound or substituted β-lactam compound or a pharmaceutically acceptable salt thereof.

10. The composition of claim 7, wherein said at least one cholesterol lowering compound is ezetimibe.

11. A method of treating, reducing, or ameliorating a condition or disease selected from psychic disorders, anxiety, schizophrenia, depression, abuse of psychotropes, substance abuse, substance dependency, alcohol dependency, nicotine dependency, neuropathies, migraine, stress, epilepsy, dyskinesias, Parkinson's disease, amnesia, senile dementia, Alzheimer's disease, eating disorders, type II diabetes, gastrointestinal diseases, vomiting, diarrhea, urinary disorders, infertility disorders, inflammation, infection, cancer, neuroinflammation, atherosclerosis, Guillain-Barr syndrome, viral encephalitis, cerebral vascular incidents, and cranial trauma in a patient in need thereof, comprising: administering to said patient in need thereof an effective amount of a compound of claim 1, or a pharmaceutically acceptable salt thereof.

12. A method of treating, reducing, or ameliorating a condition or disease selected from metabolic syndrome, obesity, waist circumference, abdominal girth, type II diabetes, insulin resistance, hepatic lipidosis, fatty liver disease, neuroinflammatory disorders, cognitive disorders, psychosis, addictive behavior, gastrointestinal disorders, and cardiovascular conditions, in a patient in need thereof, comprising: administering to said patient in need thereof an effective amount of at least one compound according to claim 1, or a pharmaceutically acceptable salt thereof.

13. The method of claim 12, wherein said condition or disease is selected from metabolic syndrome, obesity, waist circumference, abdominal girth, type II diabetes, hepatic lipidosis, and fatty liver disease.

14. A method of reducing body condition score in a patient in need thereof, comprising administering to said patient in need thereof an effective amount of at least one compound according to claim 1, or a pharmaceutically acceptable salt thereof.

15. A method of partitioning energy of an animal away from fat deposition toward protein accretion, comprising administering to said animal an effective amount of at least one compound according to claim 1, or a pharmaceutically acceptable salt thereof.
